(19) United States
(12) Patent Application Publication
(10) Pub. No.: US 2026/0219571 A1
(43) Pub. Date: Jul. 30, 2026

(54) SULFONIUM SALT, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/451,279

(22) Filed: Jan. 16, 2026

(30) Foreign Application Priority Data
Jan. 24, 2025 (JP) 2025-010482

(51) Int. Cl. G03F 7/004; C07C 381/12; C08F 20/68; G03F 7/00; G03F 7/039; G03F 7/20

(52) U.S. Cl. CPC G03F 7/0045; C07C 381/12; C08F 20/68; G03F 7/0397; G03F 7/2004; G03F 7/70033

(57) ABSTRACT

A sulfonium salt consisting of a sulfonium cation containing a pentafluorosulfanyl group and an acid labile group of tertiary/secondary ether structure, tertiary/secondary carbonate structure or acetal structure and an aromatic carboxylic acid anion is provided. A chemically amplified resist composition comprising the sulfonium salt as a quencher has a high sensitivity and resolution, and forms a resist film with improved lithography properties including LWR, CDU, EL and DOF as well as collapse resistance.

### CROSS-REFERENCE TO RELATED APPLICATION

This non-provisional application claims priority under 35 U.S.C. § 119 (a) on Patent Application No. 2025-010482 filed in Japan on Jan. 24, 2025, the entire contents of which are hereby incorporated by reference.

### TECHNICAL FIELD

This invention relates to a sulfonium salt, a chemically amplified resist composition, and a patterning process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and line width roughness (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the ArF lithography are typically based on (meth)acrylate polymers having acid labile groups. These acid labile groups undergo deprotection reaction when an acid generator capable of generating a sulfonic acid which is substituted at α-position with fluorine (referred to as “α-fluorinated sulfonic acid”, hereinafter) is used, but not when an acid generator capable of generating a sulfonic acid which is not substituted at α-position with fluorine (referred to as “α-non-fluorinated sulfonic acid”, hereinafter) or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Sulfonium salt type quenchers capable of generating carboxylic acid are known. Proposed thus far are sulfonium salts of salicylic acid and β-hydroxycarboxylic acid (Patent Document 4), salicylic acid derivatives (Patent Documents 5 and 6), fluorosalicylic acids (Patent Document 7), hydroxynaphthoeic acid (Patent Document 8), salicylic acids having an iodized aromatic substituent group introduced therein (Patent Document 9), and salicylic acids having a cyclic acetal structure introduced therein (Patent Document 10). Salicylic acid is quite effective for suppressing acid diffusion due to the intramolecular hydrogen bond of carboxy group and hydroxy group.

It is pointed out that the agglomeration of a quencher causes to degrade the dimensional uniformity or CDU of resist patterns. It is thus expected that the CDU of resist patterns after development is improved by preventing the quencher from agglomerating in the resist film, for achieving a uniform distribution of the quencher.

In connection with the demand for further miniaturization, there is a problem that upon development of a positive resist film in an alkaline developer, the resist film is swollen with the developer so that pattern collapse may occur upon small-size pattern formation. To solve the problem associated with miniaturization, the development of an effective material for a new resist composition is important. It is desired to have an onium salt type quencher having a high sensitivity, fully controlled acid diffusion, acceptable solvent solubility, and effective prevention of pattern collapse.

### CITATION LIST

Patent Document 1: JP-A 2006-045311 (U.S. Pat. No. 7,482,108)
Patent Document 2: JP-A 2006-178317
Patent Document 3: JP-A 2007-114431
Patent Document 4: WO 2018/159560
Patent Document 5: JP-A 2020-203984
Patent Document 6: JP-A 2020-091404
Patent Document 7: JP-A 2020-091312
Patent Document 8: JP-A 2019-120760
Patent Document 9: JP-A 2022-077505
Patent Document 10: WO 2023/189502
Non-Patent Document 1: SPIE Vol. 6520 65203L-1 (2007)

### SUMMARY OF THE INVENTION

In conjunction with the demand for higher resolution of resist patterns, a prior art resist composition comprising an acid generator of sulfonium salt type and a quencher fails to fully suppress acid diffusion. This raises the problem that lithography properties including contrast, LWR, CDU, EL, and DOF are degraded. Another problem arising upon formation of small-size patterns is pattern collapse by swell.

An object of the invention is to provide a novel sulfonium salt and a chemically amplified resist composition comprising the sulfonium salt, the resist composition exhibiting a high sensitivity, high resolution, improved lithography properties including LWR, CDU, EL and DOF, and collapse resistance, when processed by lithography using high-energy radiation such as deep UV, EB, or EUV. Another object is to provide a pattern forming process using the resist composition.

The inventor has found that a sulfonium salt consisting of a sulfonium cation containing a pentafluorosulfanyl group and an acid labile group of tertiary/secondary ether structure, tertiary/secondary carbonate structure or acetal structure and an aromatic carboxylic acid anion has solvent solubility, and that a chemically amplified resist composition comprising the sulfonium salt as a quencher exhibits a high sensitivity, high contrast, improved lithography properties including LWR, CDU, EL and DOF, and collapse resistance upon formation of small-size patterns. The sulfonium salt contributes to restraint of defects during pattern development in alkaline developer.

In one aspect, the invention provides a sulfonium salt having the formula (1).

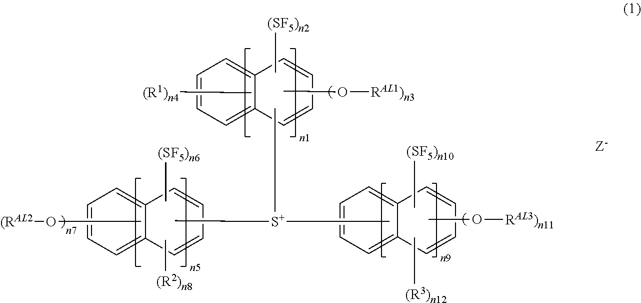

Herein n1 is 0 or 1, n2 is 0, 1 or 2, n3 is 0, 1 or 2, n4 is 0, 1 or 2, meeting 0≤n2+n3+n4≤5 when n1=0, and 0≤n2+n3+n4≤7 when n1=1, n5 is 0 or 1, n6 is 0, 1 or 2, n7 is 0, 1 or 2, n8 is 0, 1 or 2, meeting 0≤n6+n7+n8≤5 when n5=0, and 0≤n6+n7+n8≤7 when n5=1, n9 is 0 or 1, n10 is 0, 1 or 2, n11 is 0, 1 or 2, n12 is 0, 1 or 2, meeting 0≤n10+n11+n12≤5 when n9=0, and 0≤n10+n11+n12≤7 when n9=1, 1≤n2+n6+n10≤6, and 1≤n3+n7+n11≤6,

R¹, R² and R³ are each independently halogen, nitro, cyano, hydroxy, carboxy, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom; when n4=2, two R¹ may be identical or different and two R¹ may bond together to form a ring with the carbon atoms to which they are attached; when n8=2, two R² may be identical or different and two R² may bond together to form a ring with the carbon atoms to which they are attached; when n12=2, two R³ may be identical or different and two R³ may bond together to form a ring with the carbon atoms to which they are attached;
R^{AL1}, R^{AL2} and R^{AL3} are each independently an acid labile group; when n3=2, two R^{AL1} may be identical or different; when n7=2, two R^{AL2} may be identical or different; when n11=2, two R^{AL3} may be identical or different;
at least one —SF₅ group is attached to a carbon atom adjoining the carbon atom to which —O—R^{AL1}, —O—R^{AL2} or —O—R^{AL3} is attached,
two of three aromatic rings bonded to St may bond together to form a ring with the sulfur atom to which they are attached, and
Z⁻ is an aromatic carboxylic acid anion which may have a substituent.

The preferred sulfonium salt has the formula (1A):

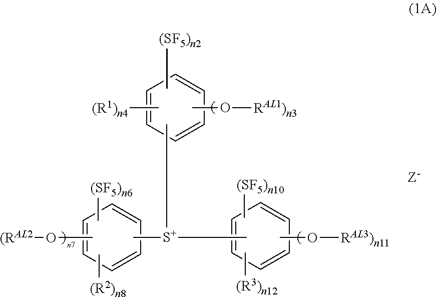

wherein n2 to n4, n6 to n8, n10 to n12, R^{AL1} to R^{AL3}, R¹ to R³, and Z⁻ are as defined above.

In a preferred embodiment, the acid labile group represented by R^{AL1}, R^{AL2} and R^{AL3} has the formula (AL-1) or (AL-2).

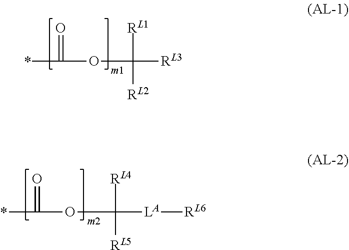

Herein m1 and m2 are each independently 0 or 1,
R^{L1} and R^{L2} are each independently a C₁-C₁₂ hydrocarbyl group, R^{L3} is hydrogen or a C₁-C₁₂ hydrocarbyl group; some —CH₂— in the hydrocarbyl group represented by R^{L1}, R^{L2} and R^{L3} may be replaced by —O— or —S—; when the hydrocarbyl group contains an aromatic ring, some or all of the hydrogen atoms on the aromatic ring may be substituted by halogen, cyano, nitro, a C₁-C₄ alkyl moiety which may contain halogen, or C₁-C₄ alkoxy moiety which may contain halogen; R^{L1} and R^{L2} may bond together to form a ring with the carbon atom to which they are attached, some —CH₂— in the ring may be replaced by —O— or —S—, with the proviso that when R^{L3} is hydrogen, R^{L1} and R^{L2} bond together to form a multiple bond-containing alicyclic structure with the carbon atom to which they are attached,
R^{L4} and R^{L5} are each independently hydrogen or a C₁-C₁₀ hydrocarbyl group, R^{L6} is a C₁-C₂₀ hydrocarbyl group, some —CH₂— in the hydrocarbyl group may be replaced by —O—or —S—; R^{L5} and R^{L6} may bond together to form a C₃-C₂₀ heterocyclic group with the carbon atom and L^{A} to which they are attached, some —CH₂— in the heterocyclic group may be replaced by —O— or —S—,
L^{A} is —O— or —S—,
* designates a point of attachment to the adjoining —O—.

In a preferred embodiment, Z⁻ is an anion having the formula (Z).

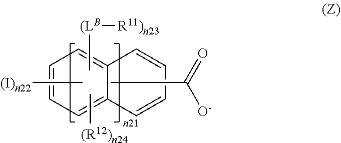

Herein n21 is 0 or 1, n22 is 0, 1, 2, 3 or 4, n23 is 0, 1 or 2, n24 is 0, 1, 2, 3 or 4, meeting 2≤n22+n23+n24≤5 when n21-0 and 2≤n22+n23+n24≤7 when n21=1,
L^{B} is a single bond, oxygen, sulfur, ester bond or carbonate bond,

R¹¹ is hydrogen or a C₁-C₂₀ hydrocarbyl group which may contain a heteroatom when L^{B} is a single bond, R¹¹ is hydrogen, a C₁-C₂₀ hydrocarbyl group (exclusive of acid labile group) which may contain a heteroatom, or acid labile group when L^{B} is oxygen, sulfur, ester bond or carbonate bond,

R¹² is halogen exclusive of iodine, nitro, cyano, pentafluorosulfanyl, a C₁-C₂₀ hydrocarbyl group which may contain a heteroatom, C₁-C₂₀ hydrocarbyloxy group which may contain a heteroatom or C₁-C₂₀ hydrocarbylthio group which may contain a heteroatom; when n24 is 2, 3 or 4, a plurality of R¹² may bond together to form a ring structure with the carbon atoms on the aromatic ring to which they are attached, some —CH₂-in the ring may be replaced by —O— or —S—.

More preferably, Z⁻ is an anion having the formula (Z1):

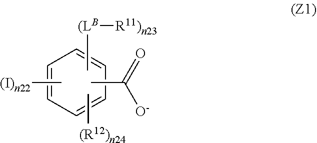

wherein n²² to n²⁴, L^{B}, R¹¹ and R¹² are as defined above.

In a preferred embodiment, L^{B} is oxygen or ester bond.

In a preferred embodiment, L^{B} is oxygen, sulfur, ester bond or carbonate bond and R¹¹ is an acid labile group.

In a preferred embodiment, the acid labile group represented by R¹¹ has the formula (AL-3) or (AL-4).

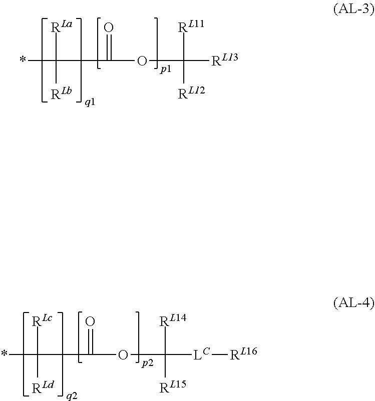

Herein p1 and p2 are each independently 0 or 1, q1 and q2 are each independently 0, 1, 2, 3 or 4,

R^{L11}, R^{L12} and R^{L13} are each independently a C₁-C₁₂ hydrocarbyl group in which some —CH₂— may be replaced by —O— or —S—; when the hydrocarbyl group contains an aromatic ring, some or all of the hydrogen atoms on the aromatic ring may be substituted by halogen, cyano, nitro, a C₁-C₄ alkyl moiety which may contain halogen, or C₁-C₄ alkoxy moiety which may contain halogen; R^{L11} and R^{L12} may bond together to form a ring with the carbon atom to which they are attached, some —CH₂— in the ring may be replaced by —O— or —S—,

R^{L14} and R^{L15} are each independently hydrogen or a C₁-C₁₀ hydrocarbyl group, R^{L16} is a C₁-C₂₀ hydrocarbyl group, some —CH₂— in the hydrocarbyl group may be replaced by —O— or —S—, R^{L15} and R^{L16} may bond together to form a C₃-C₂₀ heterocyclic group with the carbon atom and L^{C} to which they are attached, some —CH₂— in the heterocyclic group may be replaced by —O— or —S—,
L^{C} is —O— or —S—,
R^{La} to R^{Ld} are each independently hydrogen or a C₁-C₂₀ hydrocarbyl group which may contain a heteroatom, and
* designates a point of attachment to the adjoining oxygen or sulfur.

In another aspect, the invention provides a quencher in the form of the sulfonium salt defined herein.

In a further aspect, the invention provides a chemically amplified resist composition comprising the quencher defined above.

The resist composition may further comprise a base polymer containing a polymer comprising repeat units of at least one type selected from repeat units having the formula (a1), repeat units having the formula (a2), and repeat units having the formula (a3).

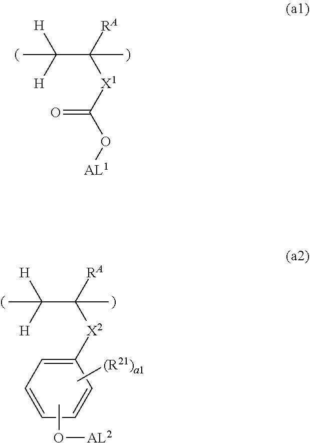

Herein R^{A} is each independently hydrogen, fluorine, methyl or trifluoromethyl,
X¹ is a single bond, phenylene group, naphthylene group, or *—C(═O)—O—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond or *—C(═O)—O—,
* designates a point of attachment to the carbon atom in the backbone,
R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when a1 is 2, 3 or 4, a plurality of R²¹ may be identical or different,
AL¹ and AL² are each independently an acid labile group, and
a1 is 0, 1, 2, 3 or 4.

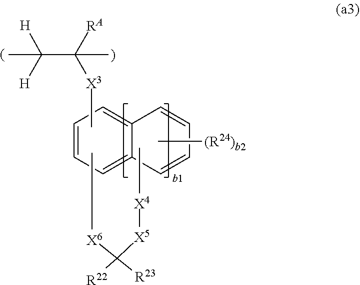

Herein b1 is 0 or 1, b2 is 0, 1, 2 or 3 when b1=0, b2 is 0, 1, 2, 3, 4 or 5 when b1=1,
R^{A} is hydrogen, fluorine, methyl or trifluoromethyl,
X³ is a single bond, *—C(═O)—O— or *—C(═O)—N(H)—, * designates a point of attachment to the carbon atom in the backbone,
X⁴ is a single bond, C₁-C₄ aliphatic hydrocarbylene group, carbonyl group, sulfonyl group or a combination thereof,
X⁵ and X⁶ are each independently oxygen or sulfur, with the proviso that X⁴ and X⁶ are attached to adjoining carbon atoms on the aromatic ring,
R²² and R²³ are each independently hydrogen or a C₁-C₂₀ hydrocarbyl group which may contain a heteroatom, R²² and R²³ may bond together to form a ring with the carbon atom to which they are attached,
R²⁴ is halogen, hydroxy, cyano, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}), R^{24A} and R^{24B} are each independently hydrogen or a C₁-C₆ hydrocarbyl group; when b2 is 2 or more, a plurality of R²⁴ may be identical or different and a plurality of R²⁴ may bond together to form a ring with the carbon atoms on the aromatic ring to which they are attached.

In a preferred embodiment, the polymer further comprises repeat units having the formula (b1) or (b2).

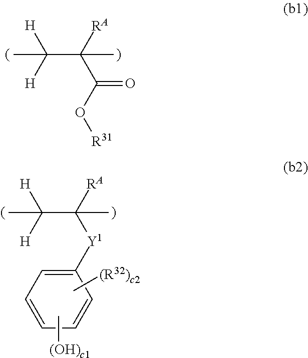

Herein R^{A} is each independently hydrogen, fluorine, methyl or trifluoromethyl,
Y¹ is a single bond or *—C(═O)—O—, * designates a point of attachment to the carbon atom in the backbone,
R³¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when c2 is 2, 3 or 4, a plurality of R³² may be identical or different,
c1 is 1, 2, 3 or 4, c2 is 0, 1, 2, 3 or 4, and 1≤c1+c2≤5.

In a preferred embodiment, the polymer further comprises repeat units of at least one type selected from repeat units having the formula (c1), repeat units having the formula (c2), repeat units having the formula (c3), repeat units having the formula (c4), and repeat units having the formula (c5).

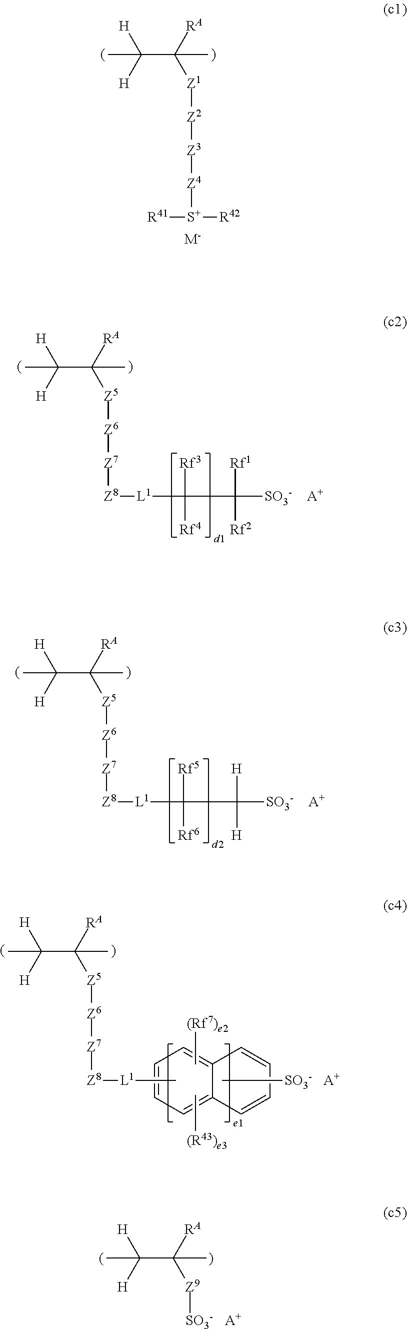

Herein d1 and d2 are each independently 0, 1, 2 or 3, e1 is 0 or 1, e2 is 0, 1, 2, 3 or 4, e3 is 0, 1, 2, 3 or 4, meeting 0≤e2+e3≤4 when e1=0 and 0≤e2+e3≤6 when e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—N(H)—Z²¹—, or **—O—Z²¹—, Z²¹ is a C₁-C₆ aliphatic hydrocarbylene group, phenylene group or a divalent group obtained by combining the foregoing, which may contain halogen, carbonyl moiety, ester bond, ether bond or hydroxy moiety,
Z³ is a single bond, ether bond, ester bond, sulfonate ester bond, amide bond, sulfonamide bond, carbonate bond or carbamate bond,
Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety,
Z⁵ is each independently a single bond, optionally substituted phenylene group, optionally substituted naphthylene group, *—C(═O)—O—Z⁵¹— or *—C(═O)—N(R)—Z⁵¹—, Z⁵¹ is a C₁-C₁₀ aliphatic hydrocarbylene group, phenylene group or naphthylene group, the aliphatic hydrocarbylene group may contain halogen, hydroxy, ether bond, ester bond or lactone ring, R is hydrogen or a C₁-C₁₀ hydrocarbyl group which may contain a heteroatom,
Z⁶ is a single bond, ether bond, ester bond, sulfonate ester bond, amide bond, sulfonamide bond, carbonate bond or carbamate bond,
Z⁷ is each independently a single bond, ***—Z⁷¹—C(═O)—O—, ***—C(═O)—N(R)—Z⁷¹— or ***—O—Z⁷¹—, Z⁷¹ is a C₁-C₂₀ hydrocarbylene group which may contain a heteroatom, R is as defined above,
Z⁸ is each independently a single bond, ****—Z⁸¹—C(═O)—O—, ****—C(═O)—N(R)—Z⁸¹— or ****—O—Z⁸¹—, Z⁸¹ is a C₁-C₂₀ hydrocarbylene group which may contain a heteroatom, R is as defined above,
Z⁹ is a single bond, methylene, ethylene, phenylene, fluorinated phenylene, trifluoromethyl-substituted phenylene, *—C(═O)—O—Z⁹¹—, *—C(═O)—N(R)—Z⁹¹— or *—O—Z⁹¹—, Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy, R is as defined above,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, C₁-C₆ fluorinated alkoxy group, C₁-C₆ fluorinated alkylthio group, or pentafluorosulfanyl; when e2 is 2, 3 or 4, a plurality of Rf⁷ may be identical or different,
R⁴¹ and R⁴² are each independently a C₁-C₂₀ hydrocarbyl group which may contain a heteroatom, R⁴¹ and R⁴² may bond together to form a ring with the sulfur atom to which they are attached,
R⁴³ is halogen exclusive of fluorine, or a C₁-C₂₀ hydrocarbyl group which may contain a heteroatom; when e3 is 2, 3 or 4, a plurality of R⁴³ may be identical or different and a plurality of R⁴³ may bond together to form a ring with the carbon atoms to which they are attached,
M⁻ is a non-nucleophilic counter ion, and
A⁺ is an onium cation.

The resist composition may further comprise an organic solvent, a photoacid generator capable of generating a strong acid, a quencher other than the quencher defined herein, and/or a surfactant.

In a still further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser, ArF excimer laser, EB, or EUV of wavelength 3 to 15 nm.

### Advantageous Effects of the Invention

When processed by lithography, a chemically amplified resist composition comprising the inventive sulfonium salt as a quencher exhibits a high sensitivity and contrast and forms a resist pattern having improved lithography properties including LWR, CDU, EL and DOF as well as minimal development defectiveness.

### DESCRIPTION OF THE PREFERRED EMBODIMENT

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the broken line ( - - - ) and asterisk (*) designate a point of attachment or valence bond. Me stands for methyl and Ac for acetyl. As used herein, the term “fluorinated” refers to a fluorine-substituted or fluorine-containing compound or group, and “iodized” refers to an iodine-substituted or iodine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity
EL: exposure latitude
DOF: depth of focus

### [Sulfonium Salt]

One embodiment of the invention is a sulfonium salt having the formula (1).

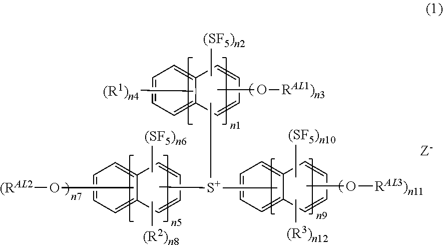

In formula (1), n1 is 0 or 1. The relevant structure is a benzene ring when n1=0, and a naphthalene ring when n1=1. The benzene ring corresponding to n1=0 is preferred from the aspect of solvent solubility. The subscript n2 is 0, 1 or 2, and n3 is 0, 1 or 2. The subscript n4 is 0, 1 or 2. From the aspect of reactant availability, n4 is preferably 0 or 1. These subscripts meet 0≤n2+n3+n4≤5 when n1=0 and 0≤n2+n3+n4≤7 when n1=1.

In formula (1), n5 is 0 or 1. The relevant structure is a benzene ring when n5=0, and a naphthalene ring when n5=1. The benzene ring corresponding to n5-0 is preferred from the aspect of solvent solubility. The subscript n6 is 0, 1 or 2, and n7 is 0, 1 or 2. The subscript n8 is 0, 1 or 2. From the aspect of reactant availability, n8 is preferably 0 or 1. These subscripts meet 0≤n6+n7+n8≤5 when n5=0 and 0≤n6+n7+n8≤7 when n5=1.

In formula (1), n9 is 0 or 1. The relevant structure is a benzene ring when n9=0, and a naphthalene ring when n9=1. The benzene ring corresponding to n9=0 is preferred from the aspect of solvent solubility. The subscript n10 is 0, 1 or 2, and n11 is 0, 1 or 2. The subscript n12 is 0, 1 or 2. From the aspect of reactant availability, n12 is preferably 0 or 1. These subscripts meet 0≤n10+n11+n12≤5 when n9-0 and 0≤n10+n11+n12≤7 when n9=1.

In formula (1), the subscripts meet 1≤n2+n6+n10≤6, preferably 1≤n2+n6+n10≤3, more preferably 1≤n2+n6+n10≤2, and 1≤n3+n7+n11≤6, preferably 1≤n3+n7+n11≤3, more preferably 1≤n3+n7+n11≤2.

In formula (1), R¹, R² and R³ are each independently halogen, nitro, cyano, hydroxy, carboxy, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. Suitable halogen atoms include fluorine, chlorine, bromine and iodine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

When n4=2, two R¹ may be identical or different. When n4=2, two R¹ may bond together to form a ring with the carbon atoms to which they are attached. The ring is preferably 5 to 8-membered. When n8=2, two R² may be identical or different. When n8=2, two R² may bond together to form a ring with the carbon atoms to which they are attached. The ring is preferably 5 to 8-membered. When n12=2, two R³ may be identical or different. When n12=2, two R³ may bond together to form a ring with the carbon atoms to which they are attached. The ring is preferably 5 to 8-membered.

In formula (1), R^{AL1}, R^{AL2} and R^{AL3} are each independently an acid labile group. When n3=2, two R^{AL1} may be identical or different. When n7=2, two R^{AL2} may be identical or different. When n11=2, two R^{AL3} may be identical or different.

The preferred acid labile group has the formula (AL-1) or (AL-2).

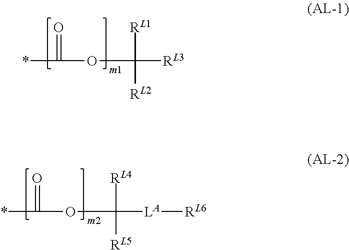

In formulae (AL-1) and (AL-2), m1 and m2 are each independently 0 or 1.

In formula (AL-1), R^{L1} and R^{L2} are each independently a C₁-C₁₂ hydrocarbyl group. R^{L3} is hydrogen or a C₁-C₁₂ hydrocarbyl group. In the hydrocarbyl group represented by R^{L1}, R^{L2} and R^{L3}, some —CH₂— may be replaced by —O— or —S—. When the hydrocarbyl group contains an aromatic ring, some or all of the hydrogen atoms on the aromatic ring may be substituted by halogen, cyano, nitro, optionally halogenated C₁-C₄ alkyl moiety or optionally halogenated C₁-C₄ alkoxy moiety. The C₁-C₁₂ hydrocarbyl group represented by R^{L1}, R^{L2} and R^{L3} may be saturated or unsaturated and straight, branched or cyclic. Examples thereof are as exemplified for the hydrocarbyl group represented by R¹, R² and R³, but of 1 to 12 carbon atoms. Also, R^{L1} and R^{L2} may bond together to form a ring with the carbon atom to which they are attached. Some —CH₂— in the ring may be replaced by —O— or —S—. When R^{L3} is hydrogen, R^{L1} and R^{L2} bond together to form a multiple bond-containing alicyclic structure with the carbon atom to which they are attached.

In formula (AL-2), R^{L4} and R¹⁵ are each independently hydrogen or a C₁-C₁₀ hydrocarbyl group. The C₁-C₁₀ hydrocarbyl group represented by R^{L4} and R^{L5} may be saturated or unsaturated and straight, branched or cyclic. Examples thereof are as exemplified above for the hydrocarbyl groups R¹, R² and R³, but of 1 to 10 carbon atoms. R^{L6} is a C₁-C₂₀ hydrocarbyl group in which some —CH₂— may be replaced by —O— or —S—. Also, R^{L5} and R^{L6} may bond together to form a C₃-C₂₀ heterocyclic group with the carbon atom and L^{A} to which they are attached. Some —CH₂— in the heterocyclic group may be replaced by —O— or —S—. The C₁-C₂₀ hydrocarbyl group represented by R^{L6} may be saturated or unsaturated and straight, branched or cyclic. Examples thereof are as exemplified above for the hydrocarbyl groups R¹, R² and R³.

In formula (AL-2), L^{A} is —O— or —S—.

Examples of the acid labile group having formula (AL-1) are shown below, but not limited thereto. Herein, * designates a point of attachment to the adjoining —O—.

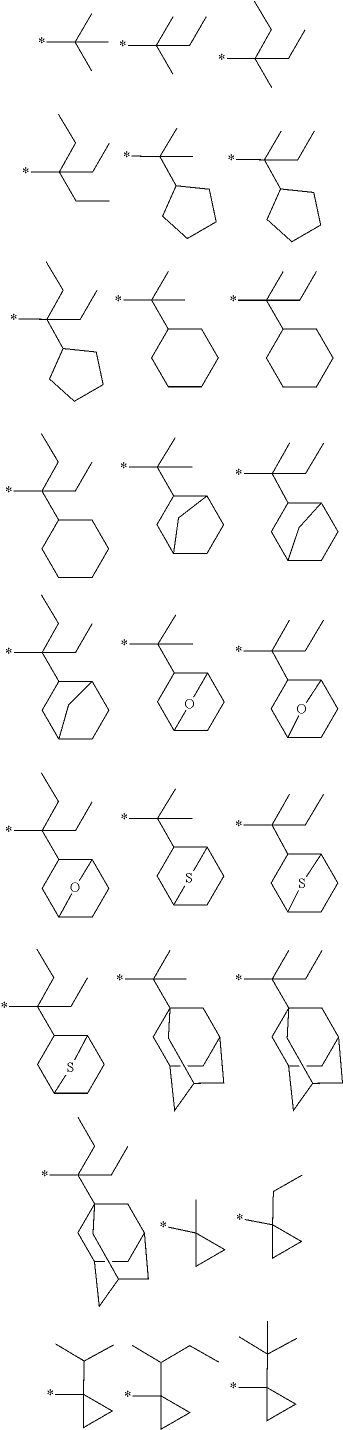

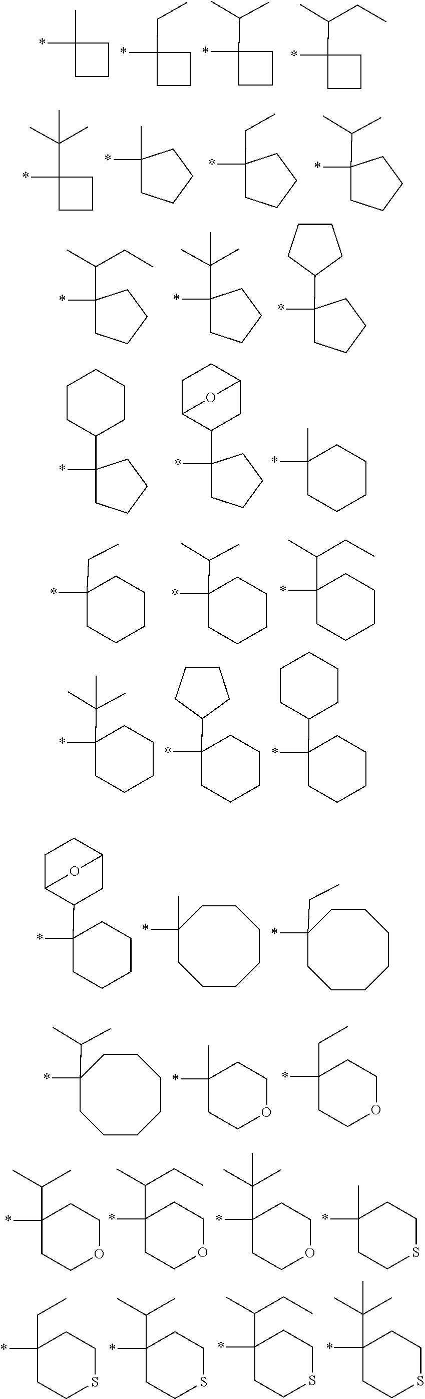

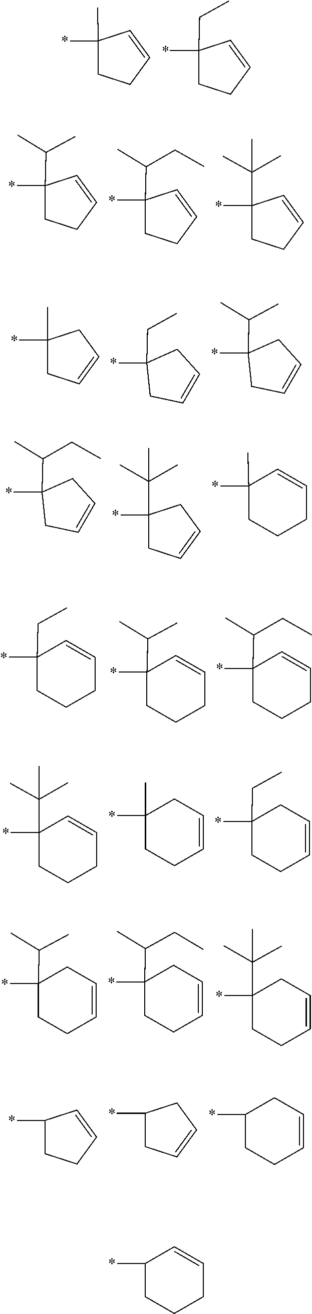

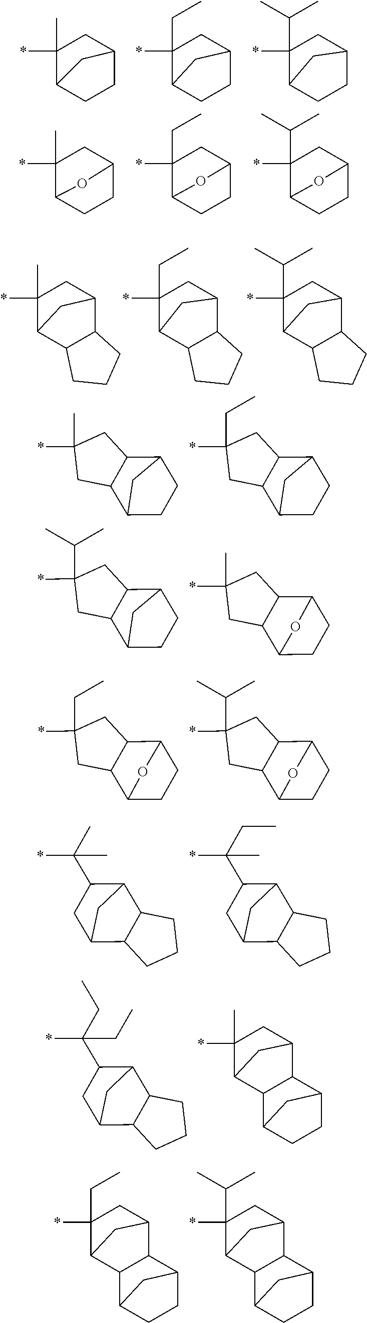

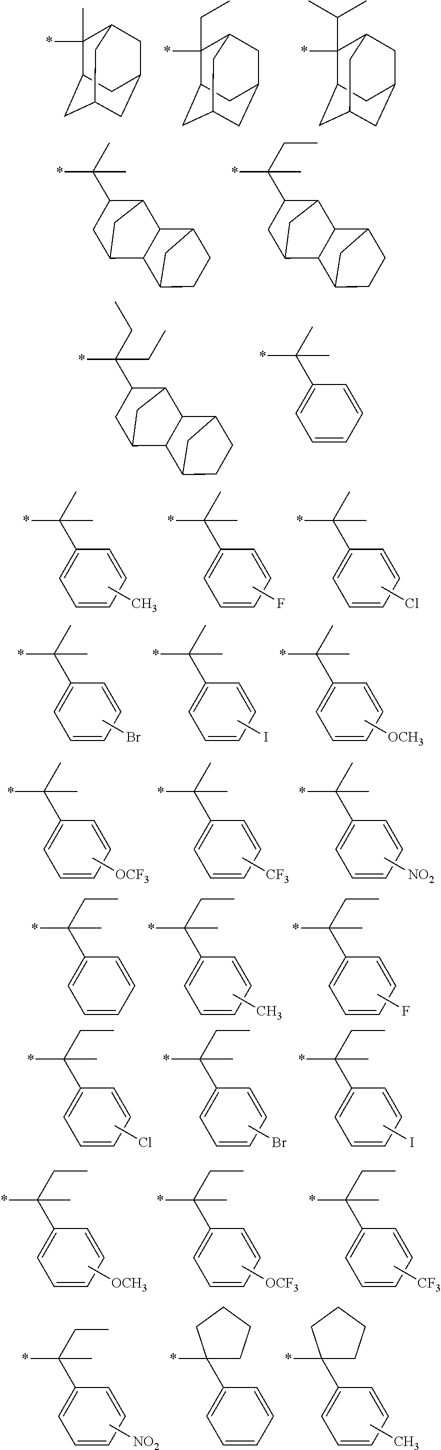

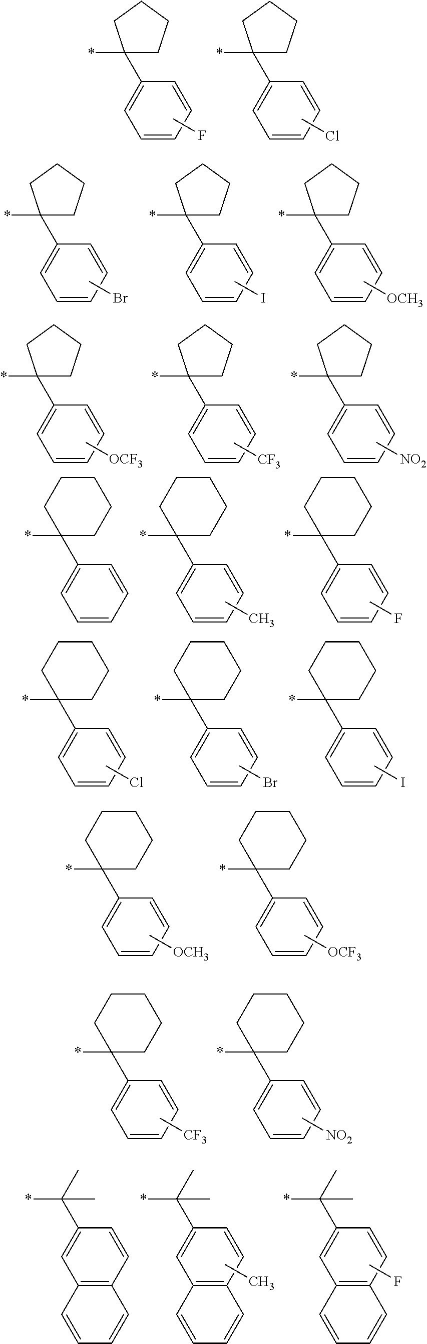

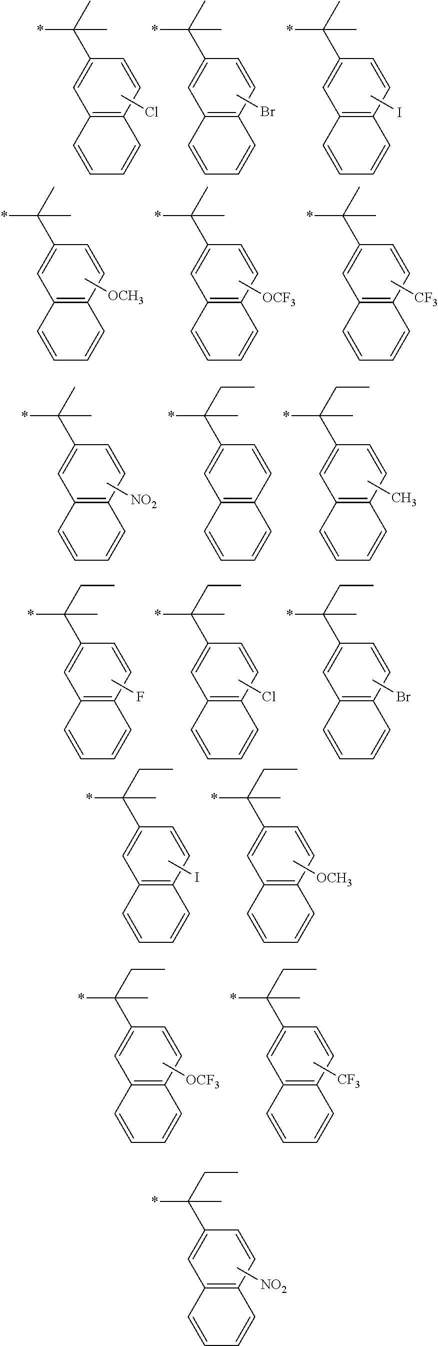

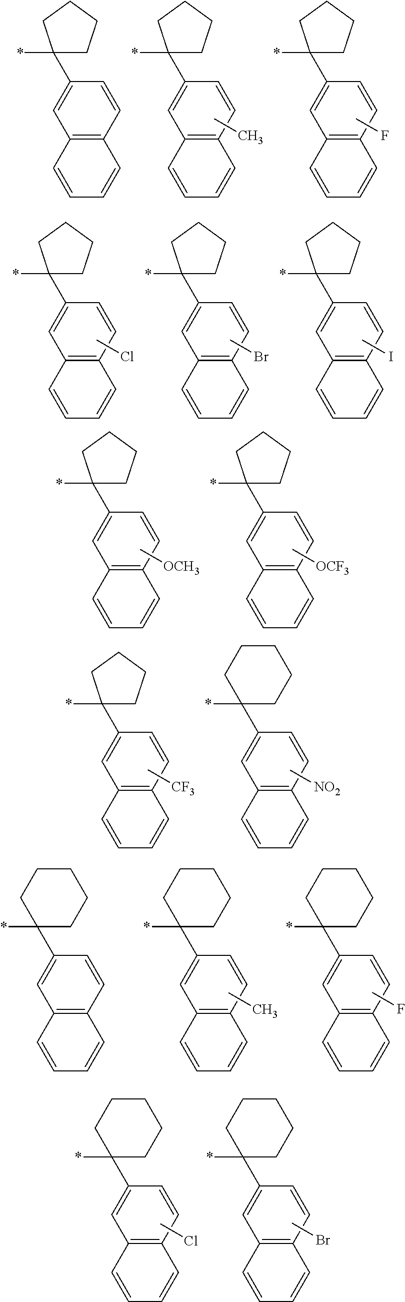

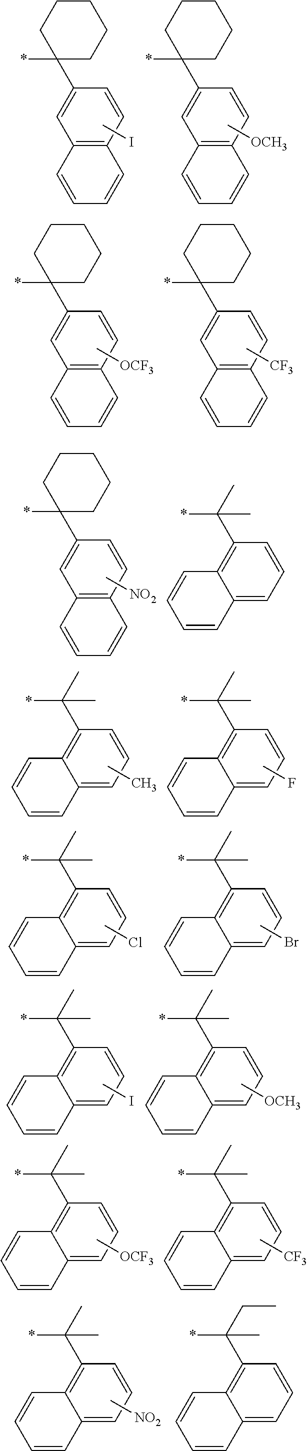

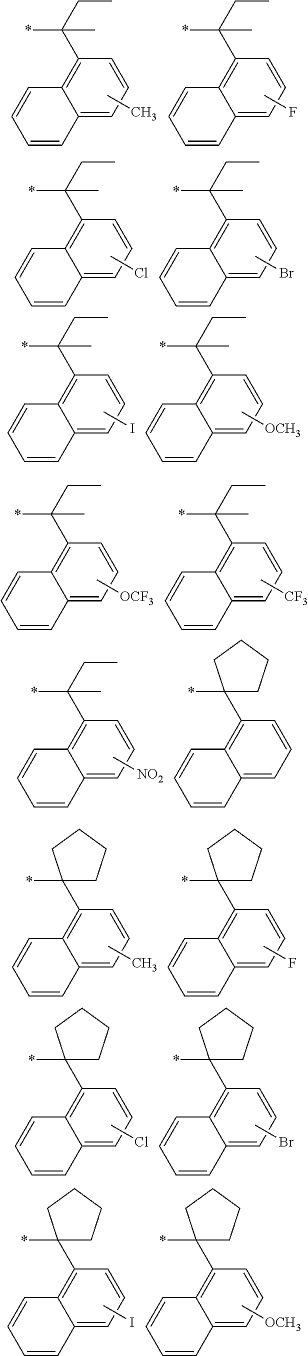

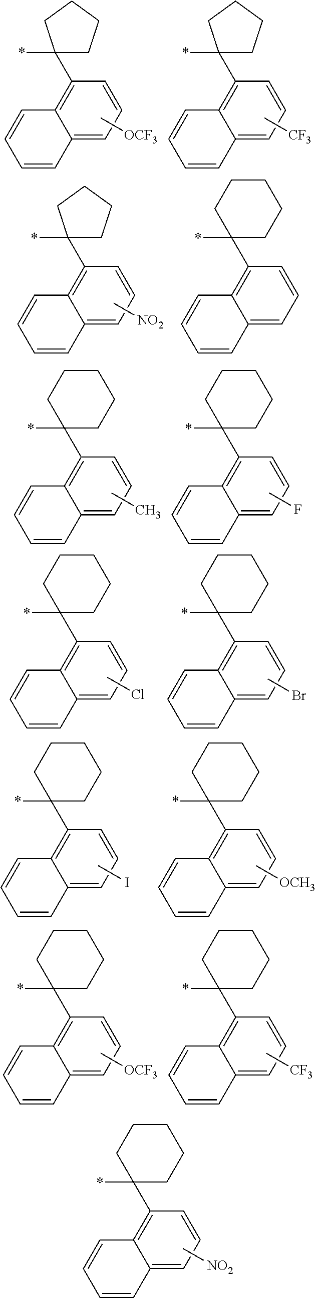

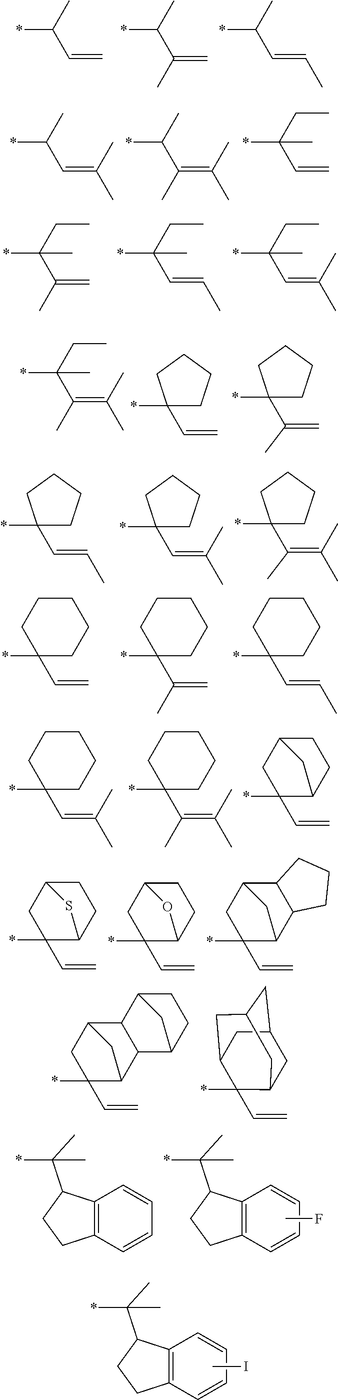

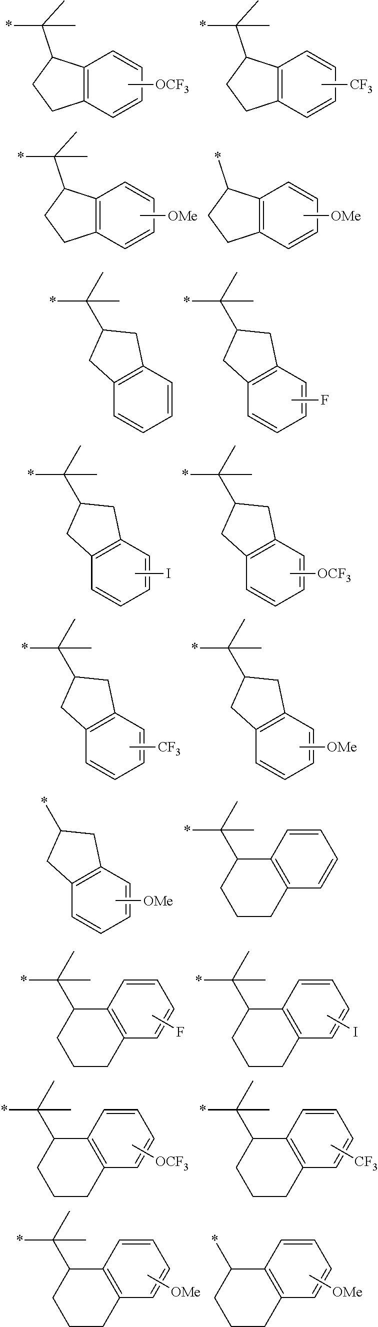

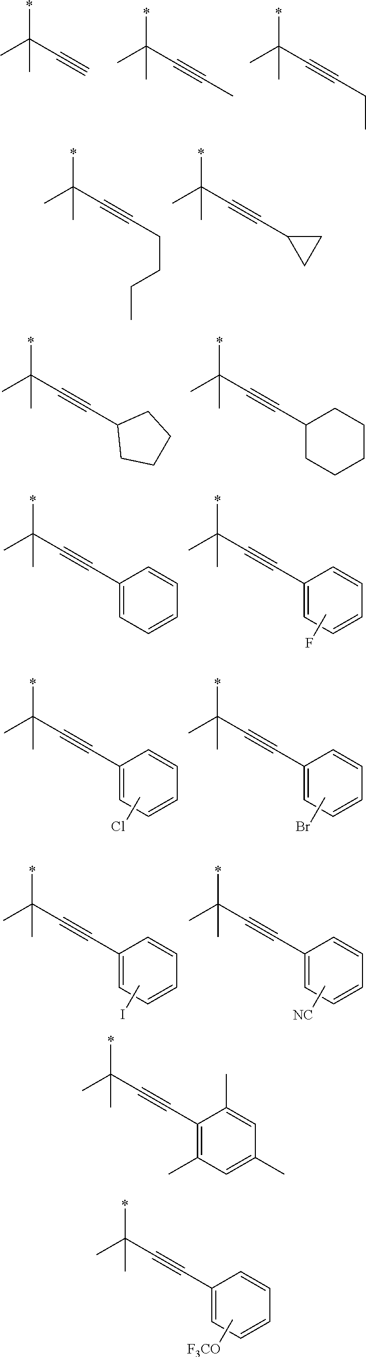

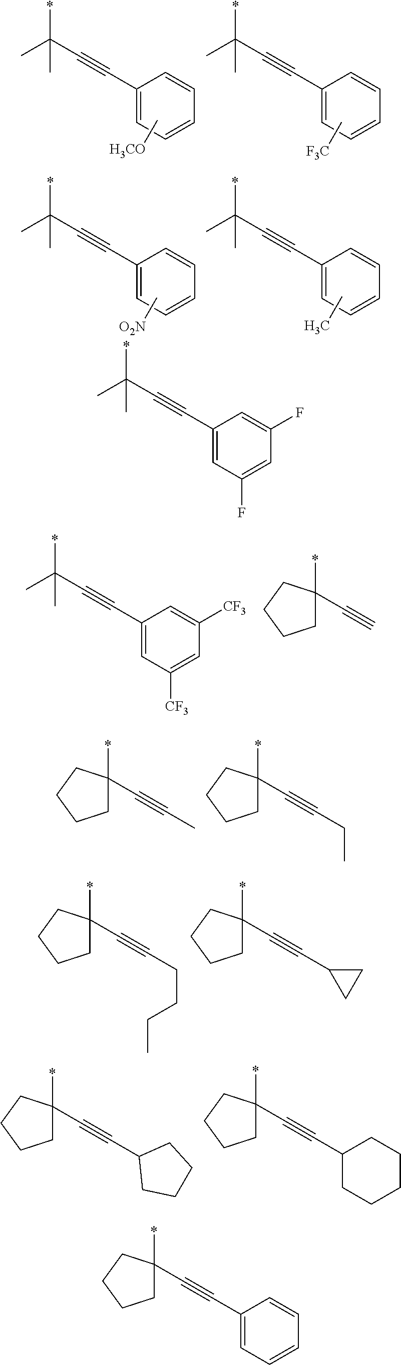

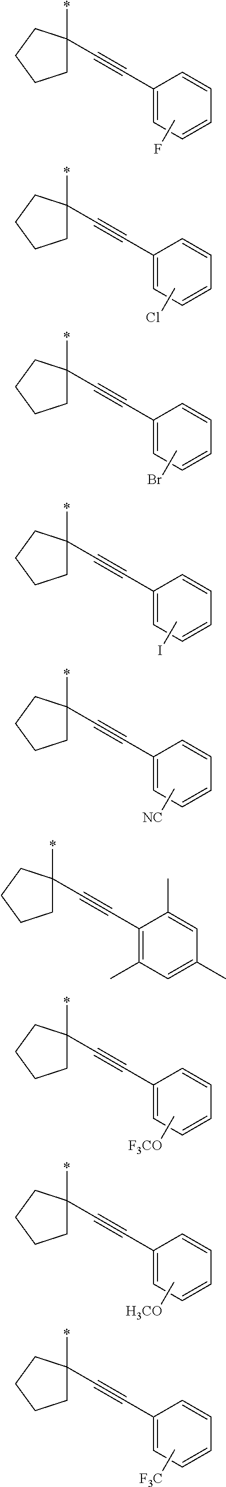

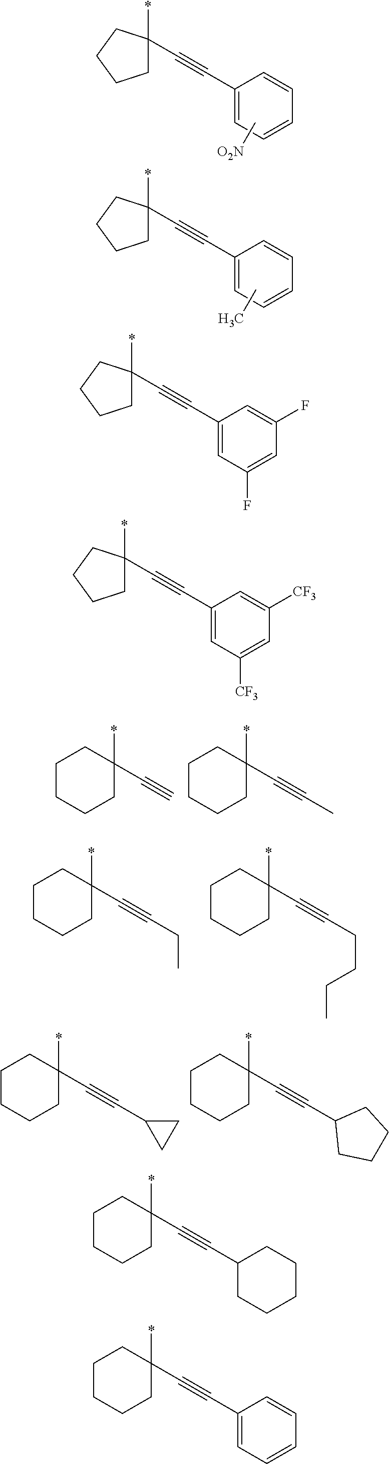

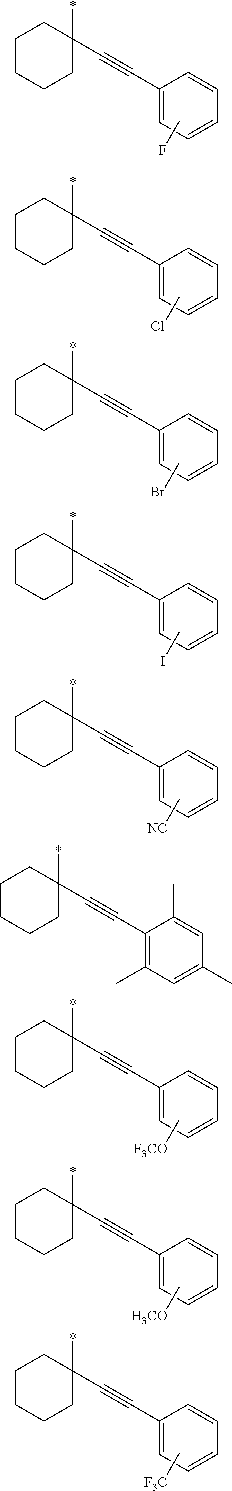

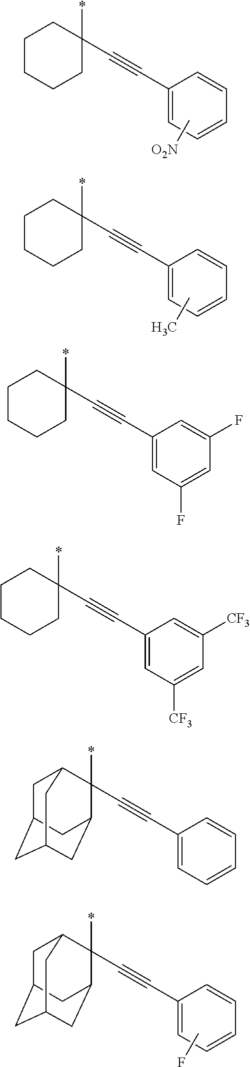

Examples of the acid labile group having formula (AL-2) are shown below, but not limited thereto. Herein, * designates a point of attachment to the adjoining —O—.

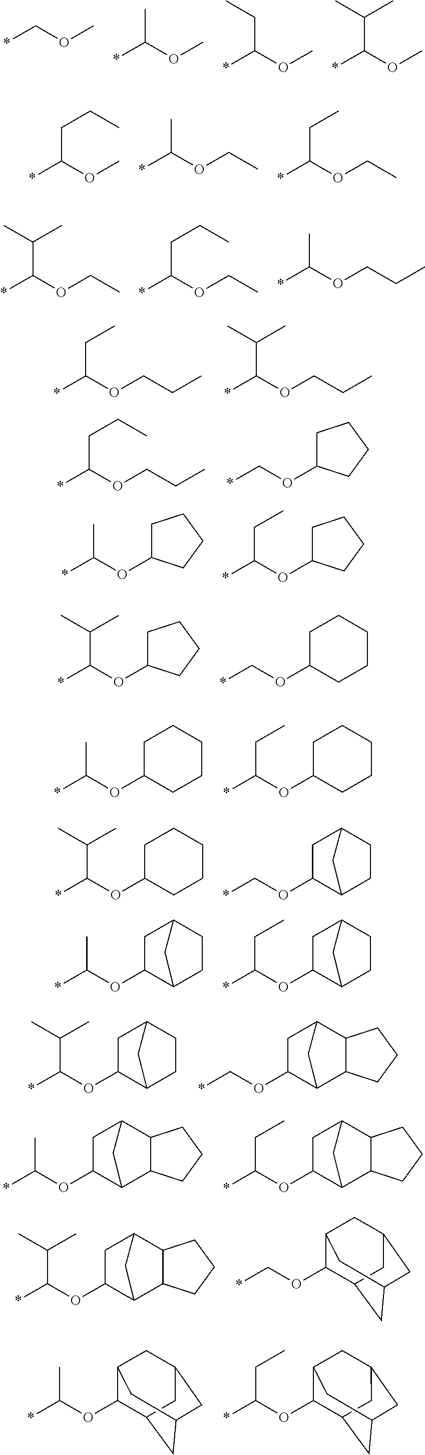

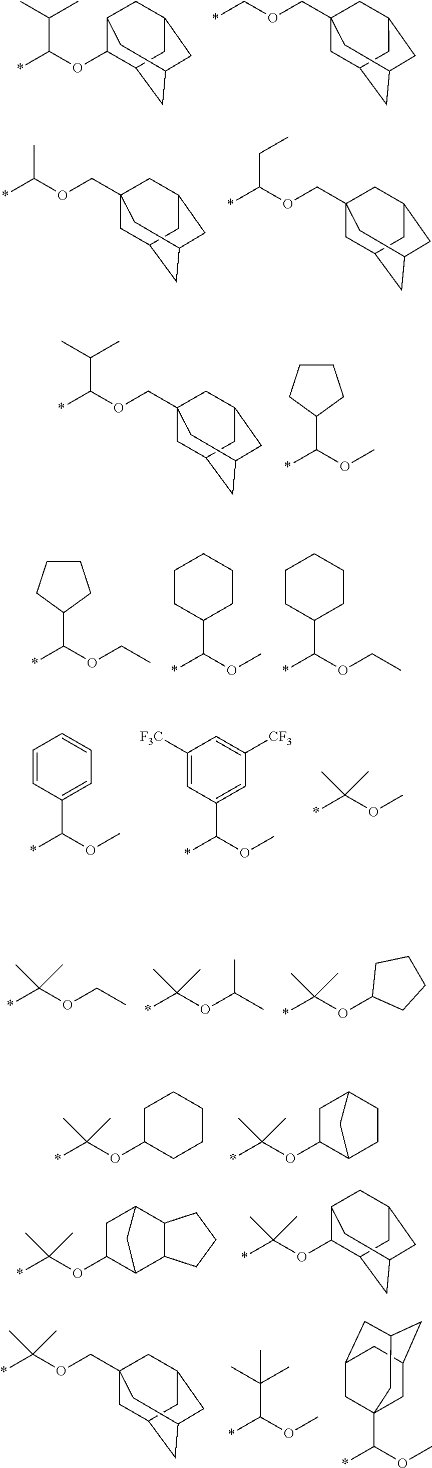

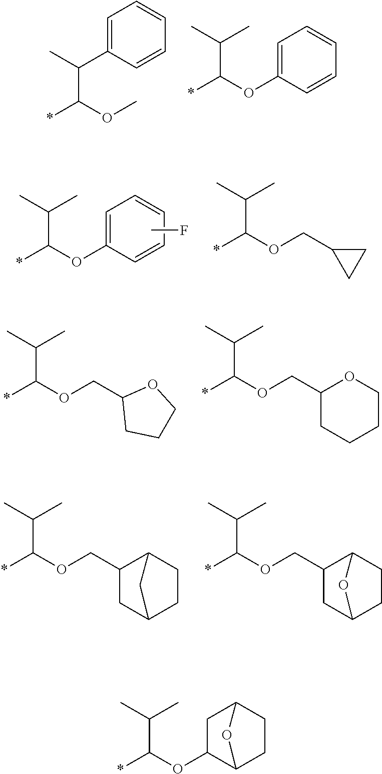

In formula (1), at least one —SF₅ group is attached to a carbon atom adjoining the carbon atom to which —O—R^{AL1}, —O—R^{AL2} or —O—R^{AL3} is attached. Now that —SF₅ group and —O—R^{AL1}, —O—R^{AL2} or —O—R^{AL3} are attached to the adjoining carbon atoms, a phenol created after deprotection of —R^{AL1}, —R^{AL2} or —R^{AL3} is increased in acidity, leading to an improved dissolution contrast.

Two of three aromatic rings bonded to S⁺ may bond together to form a ring with the sulfur atom to which they are attached. Exemplary structures of the ring are shown below.

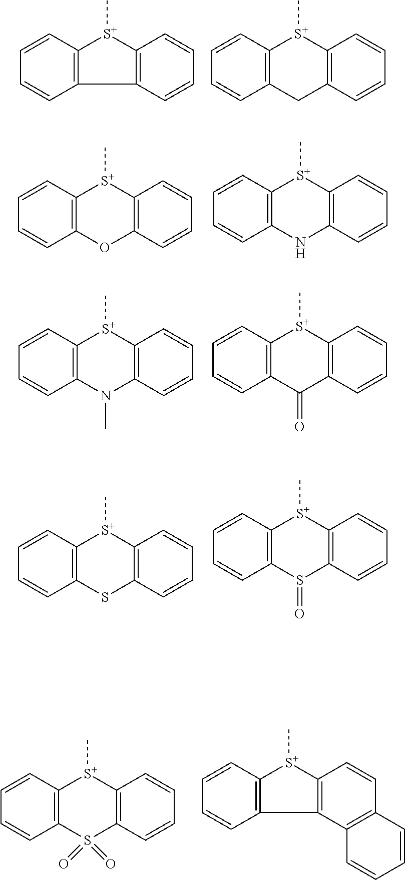

Of the sulfonium salts having formula (1), those having the formula (1A) are preferred.

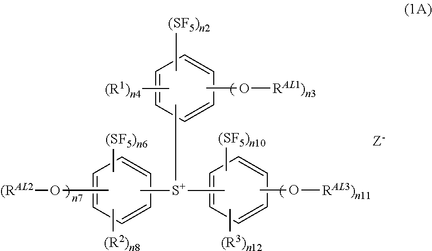

Herein n2 to n4, n6 to n8, n10 to n12, R^{AL1} to R^{AL3}, and R¹ to R³ are as defined above, and Z⁻ is described later.

Examples of the cation in the sulfonium salt having formula (1) are shown below, but not limited thereto.

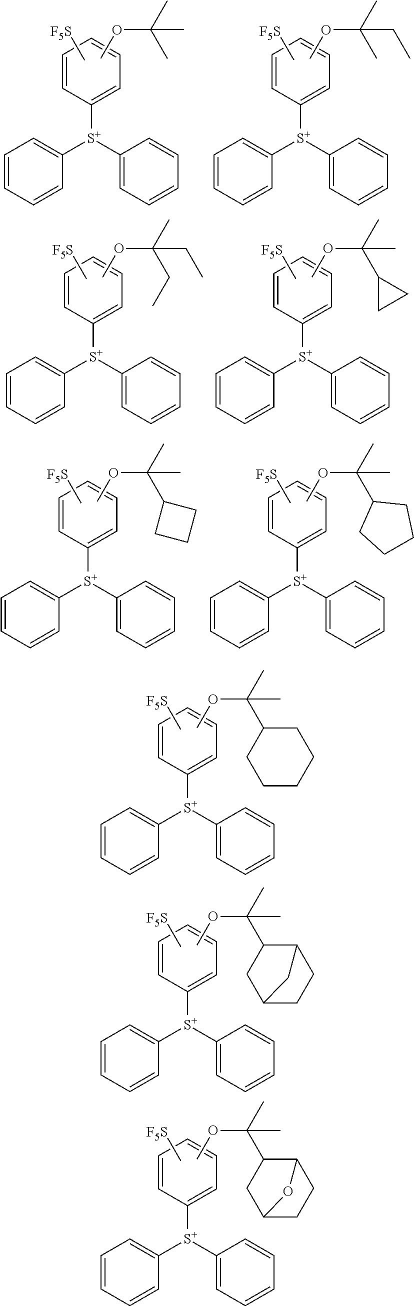

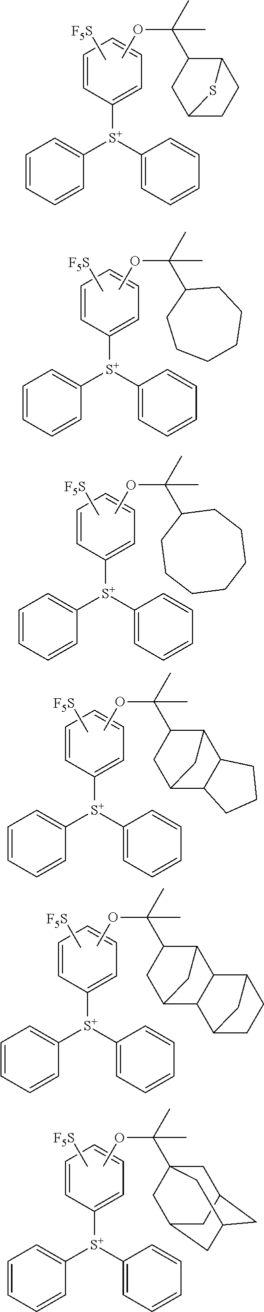

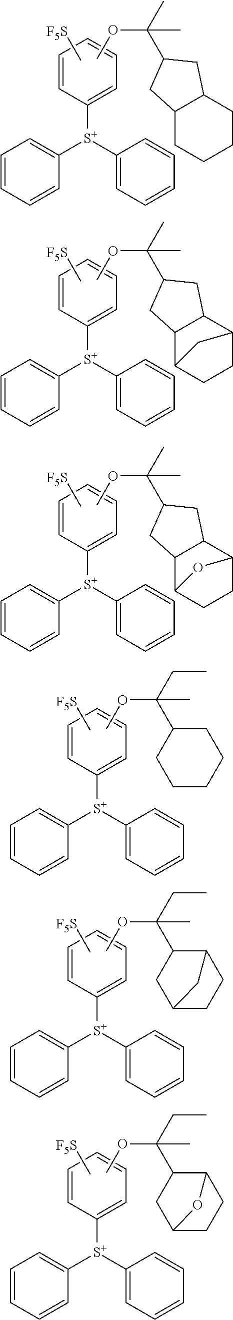

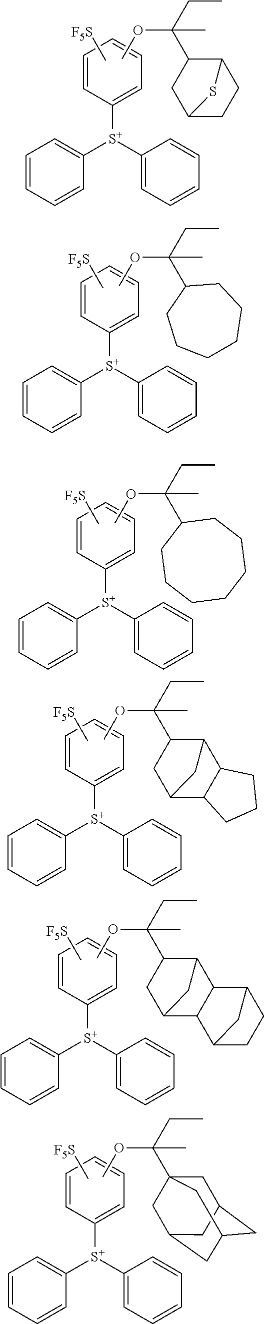

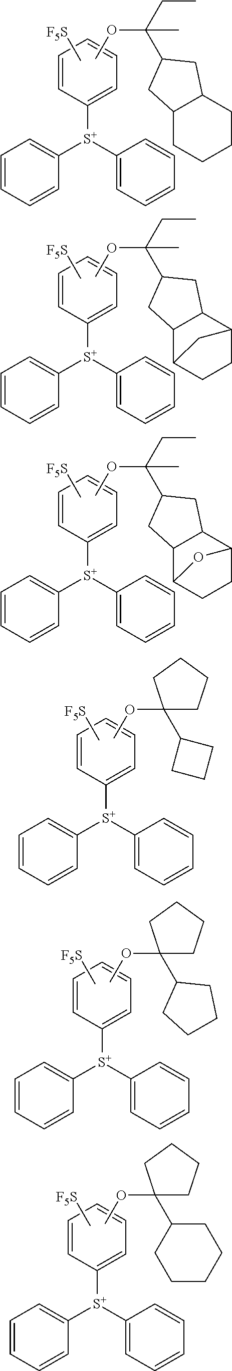

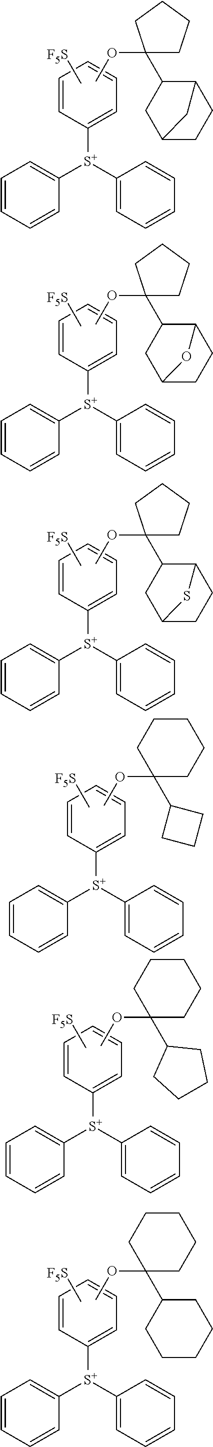

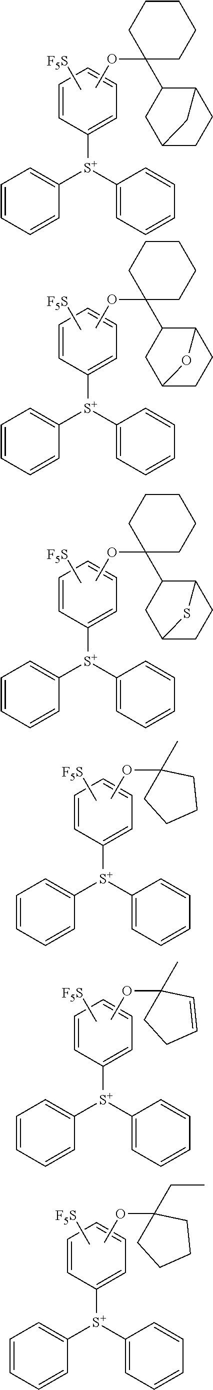

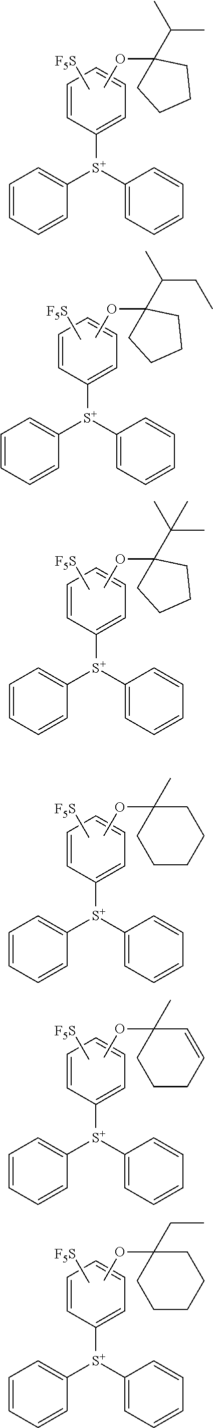

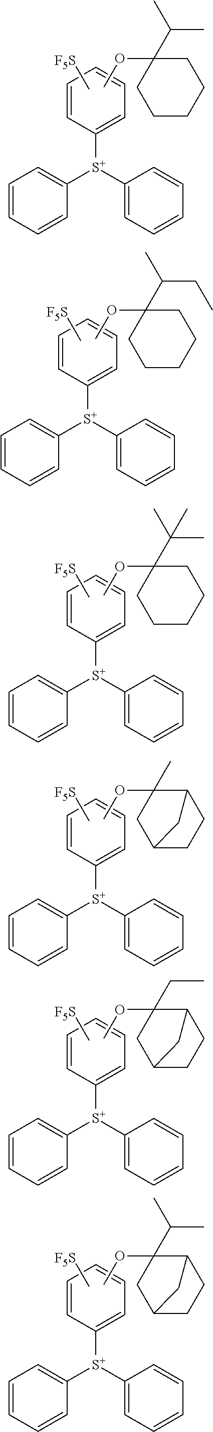

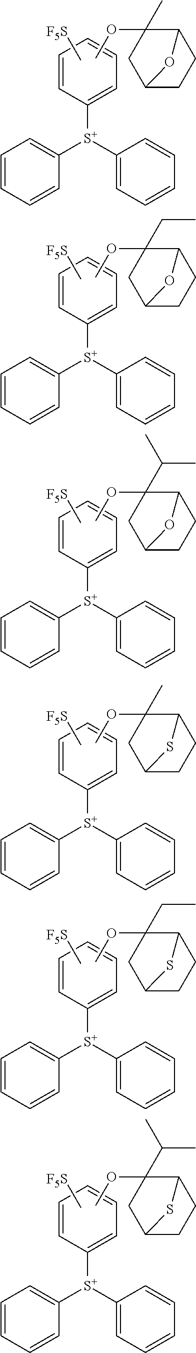

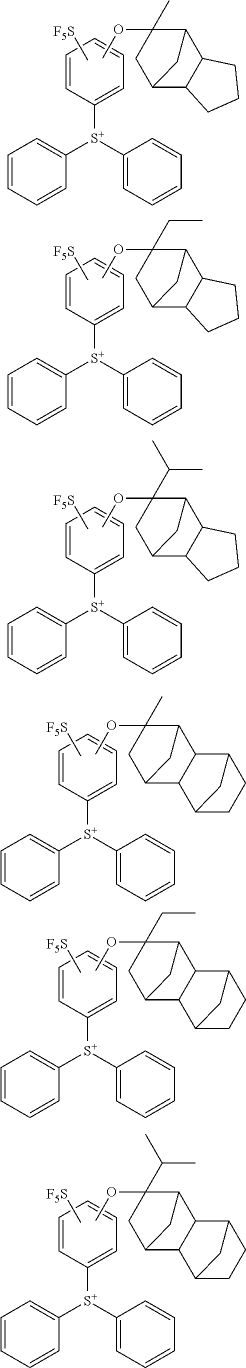

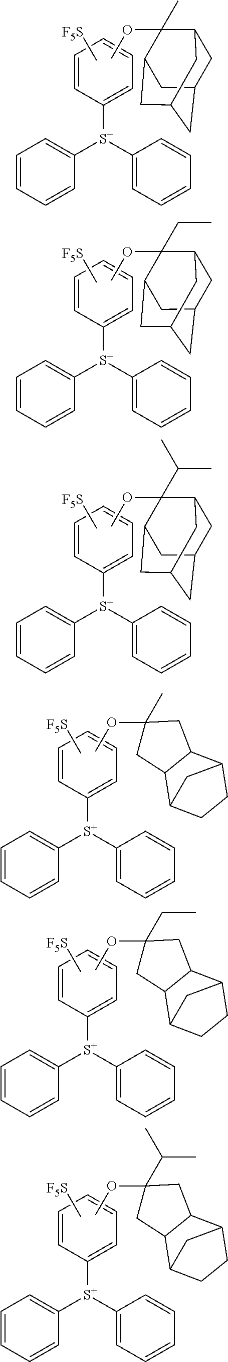

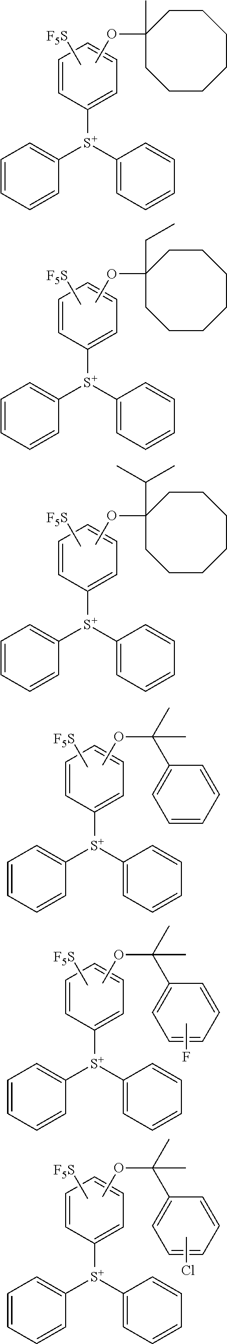

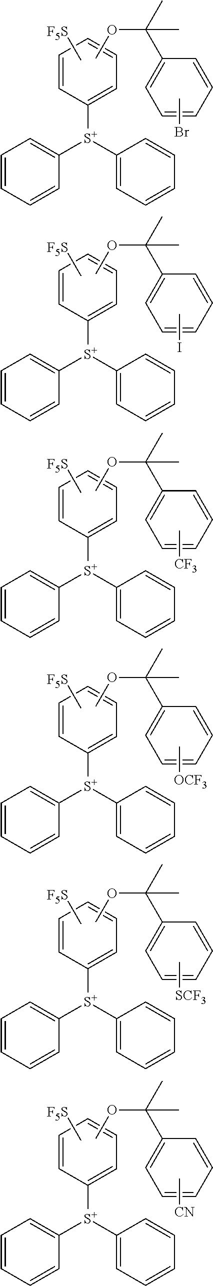

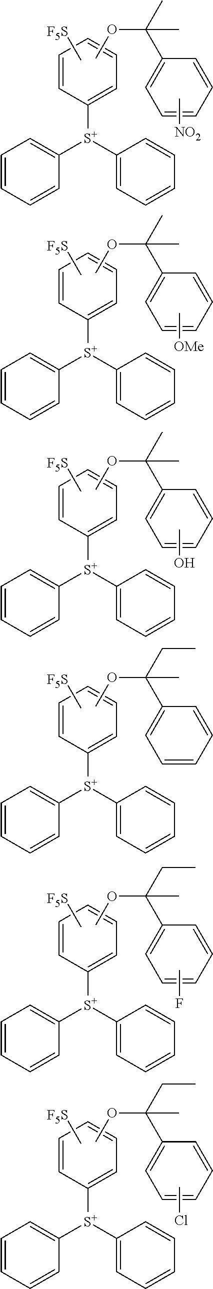

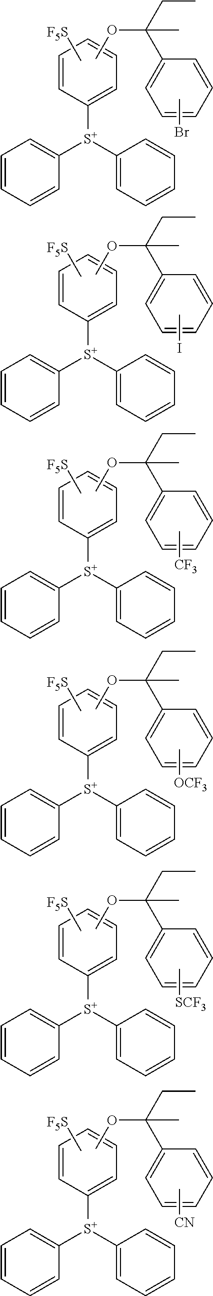

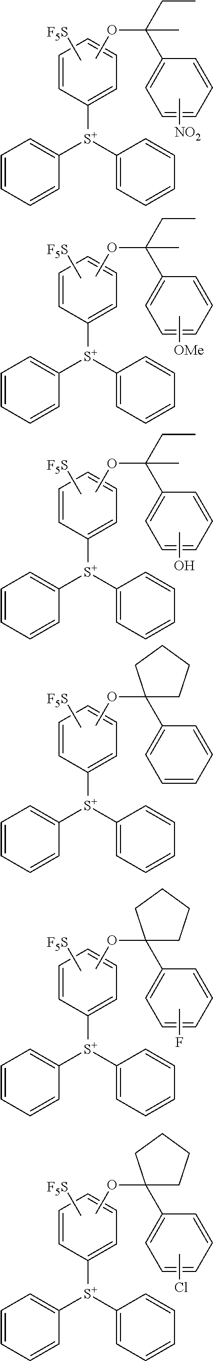

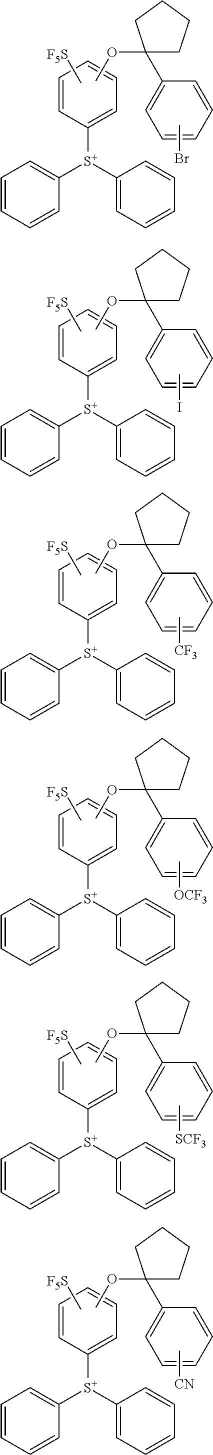

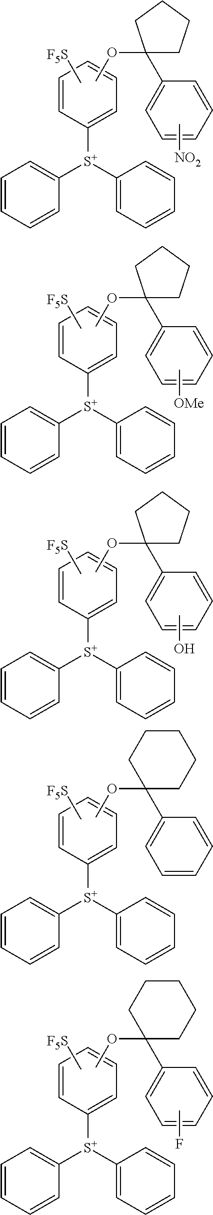

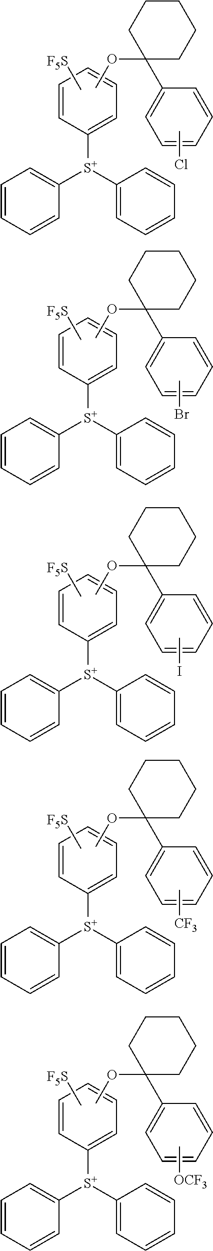

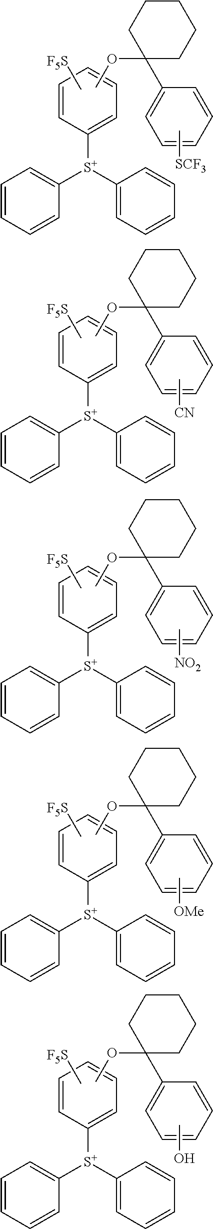

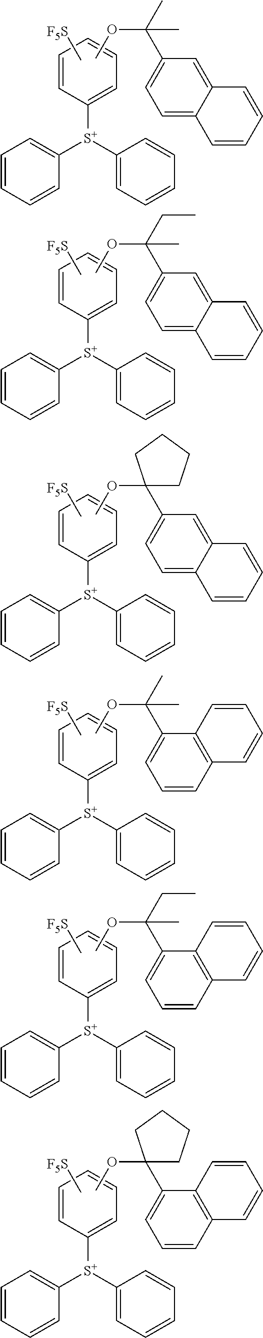

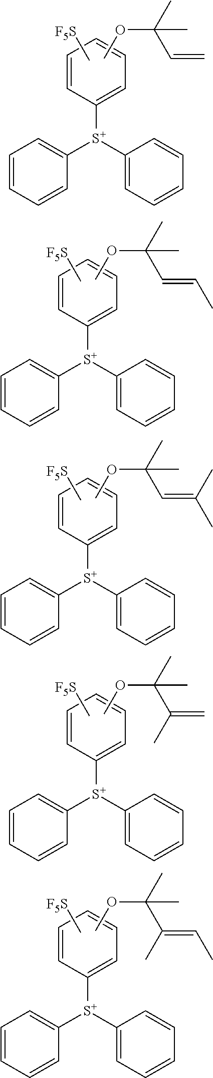

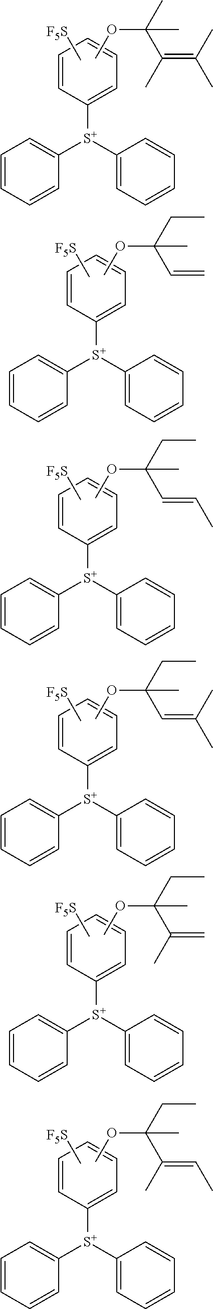

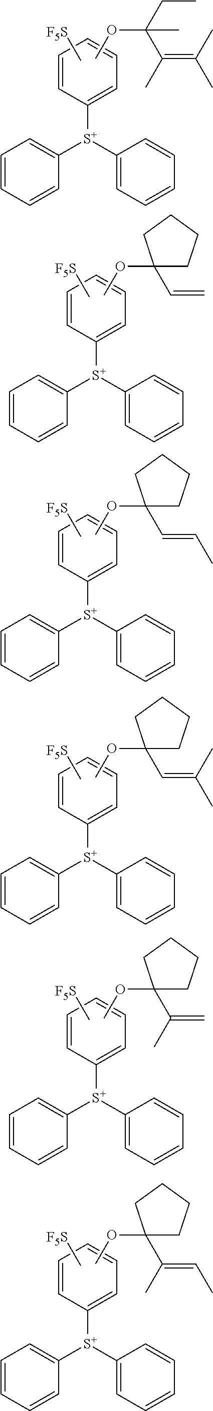

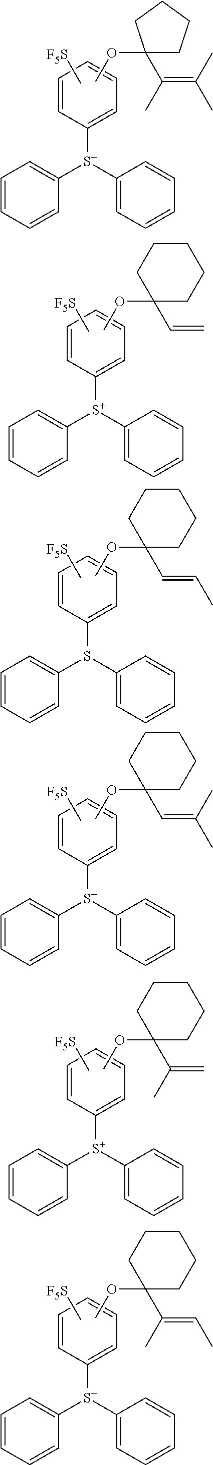

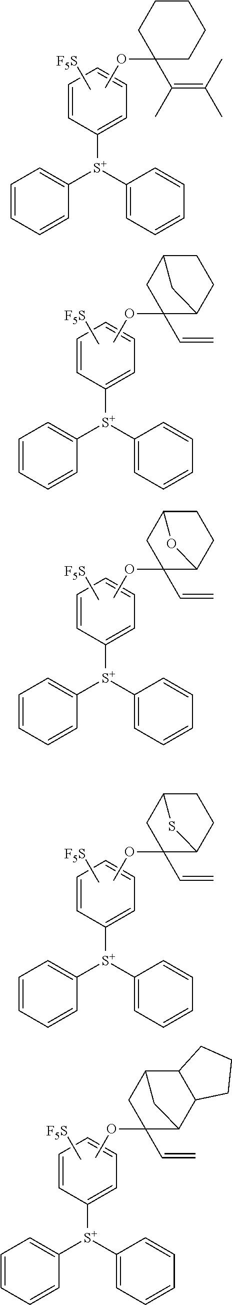

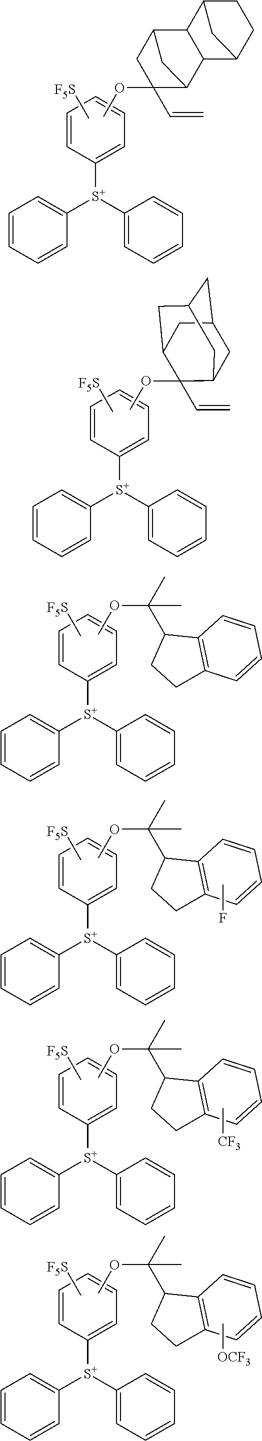

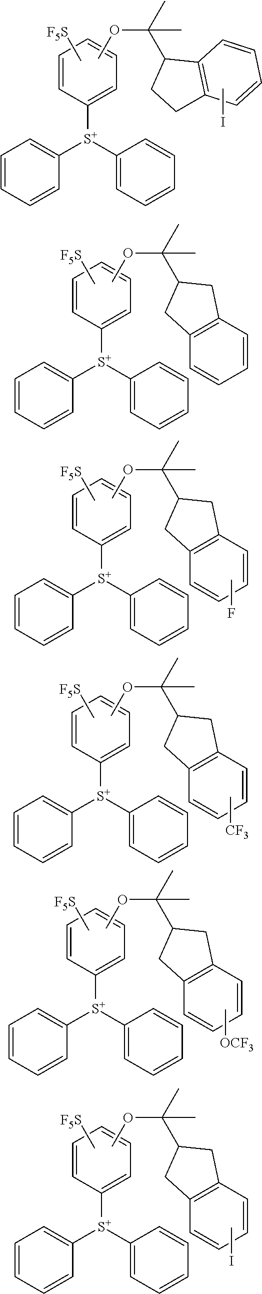

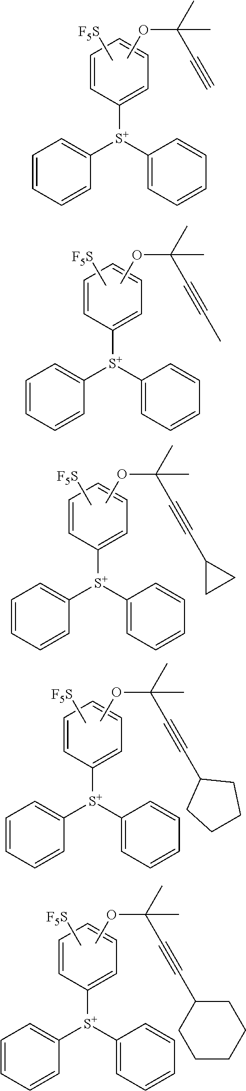

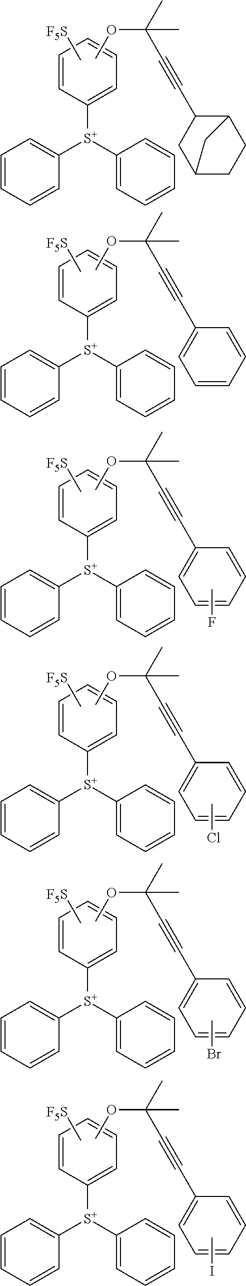

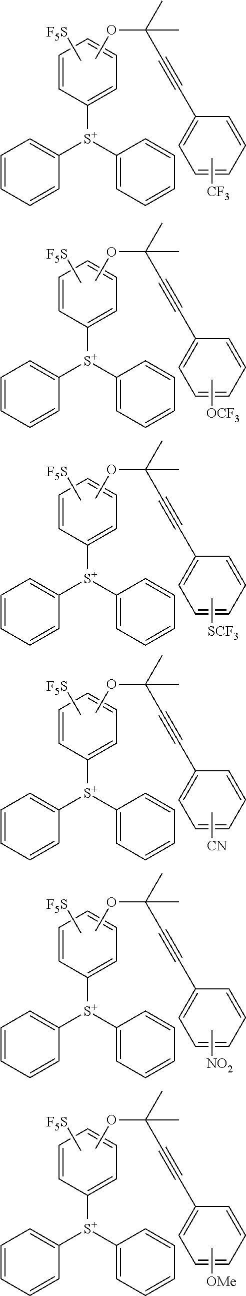

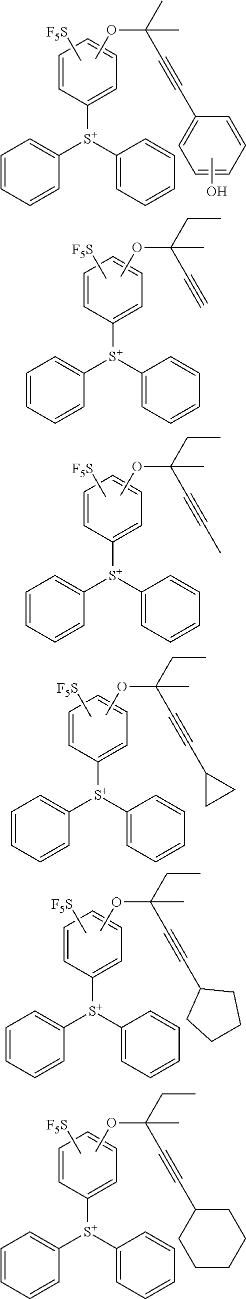

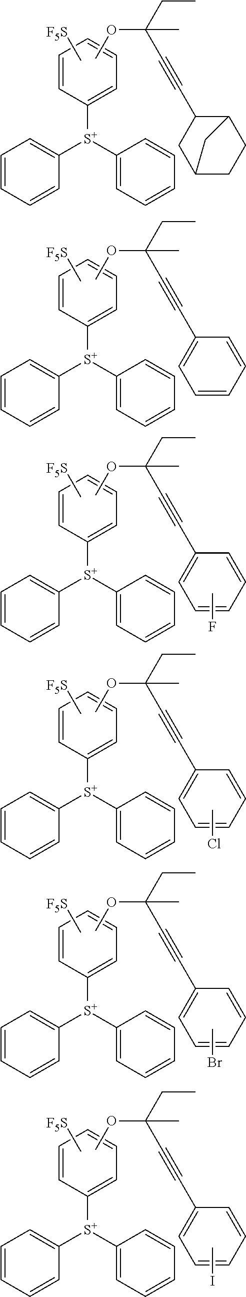

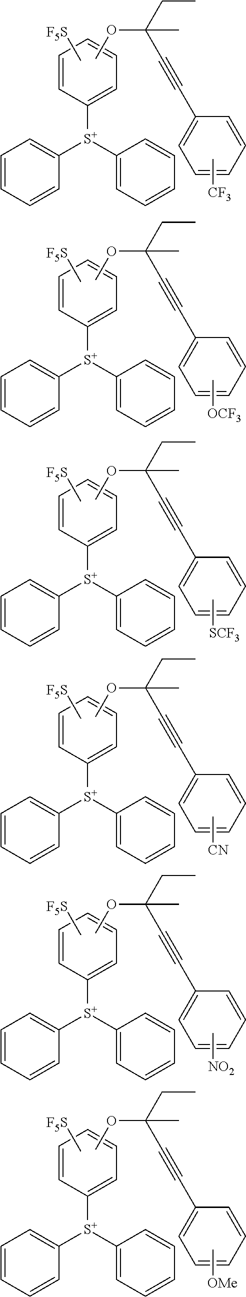

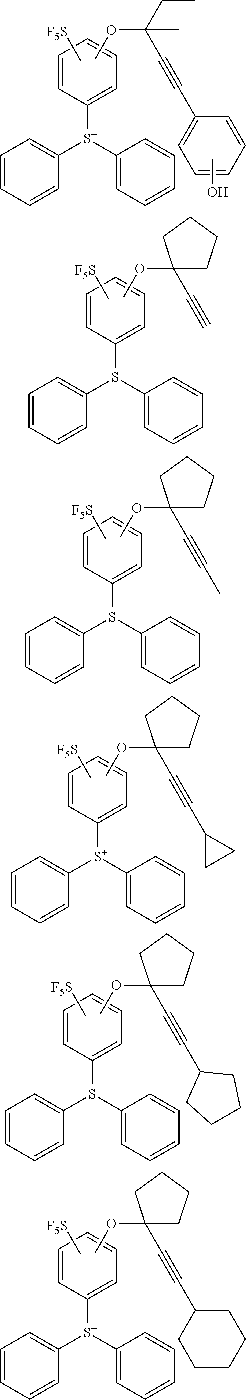

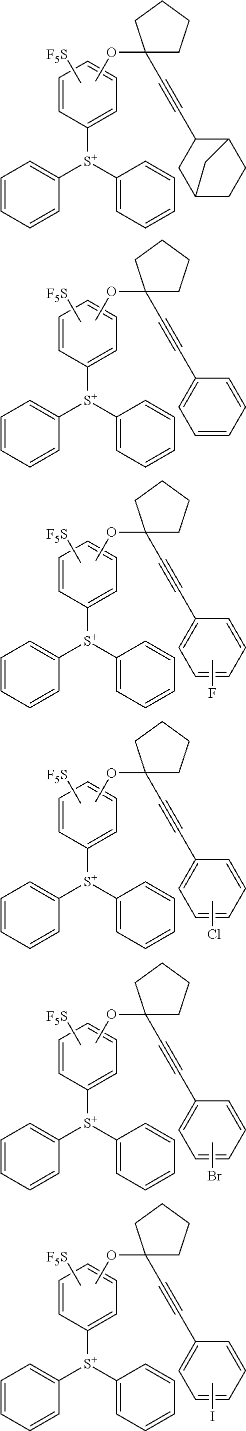

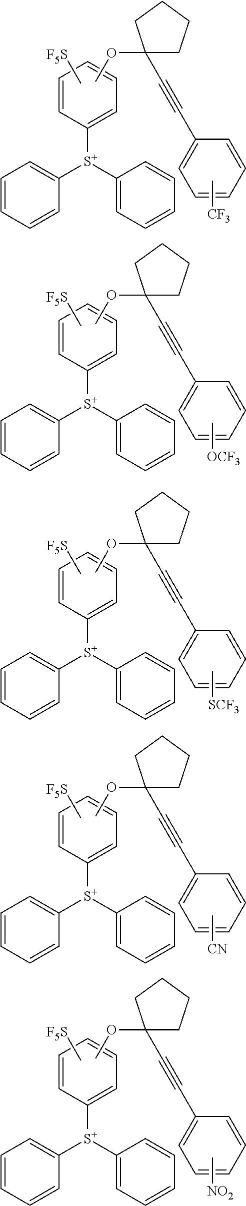

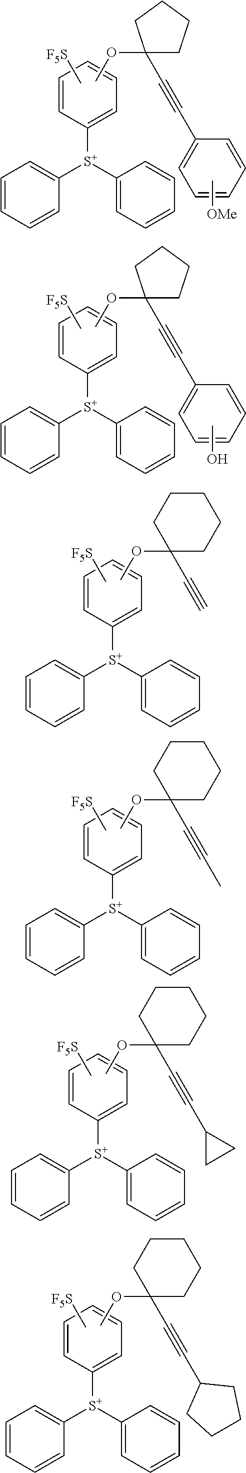

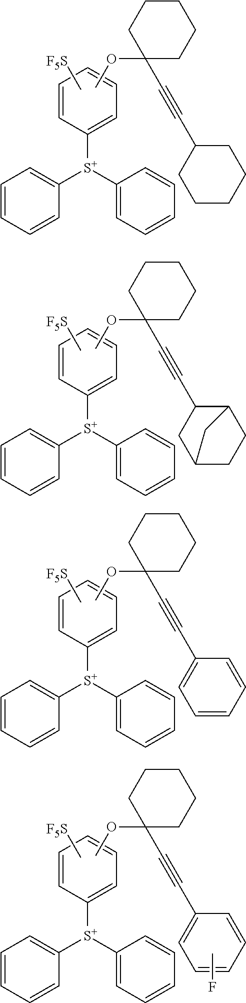

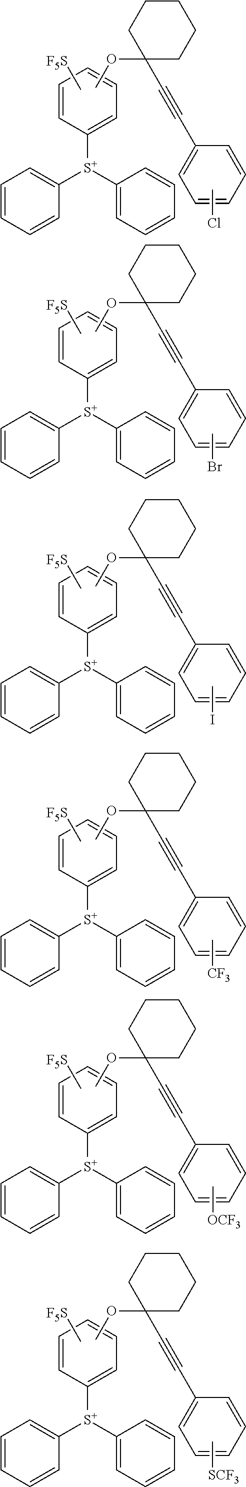

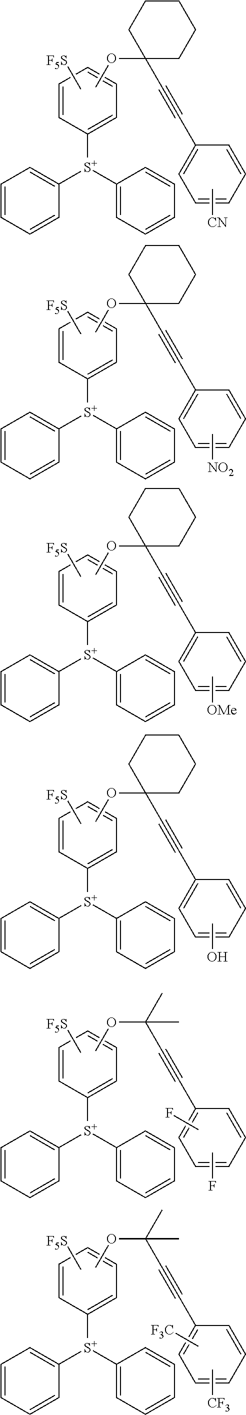

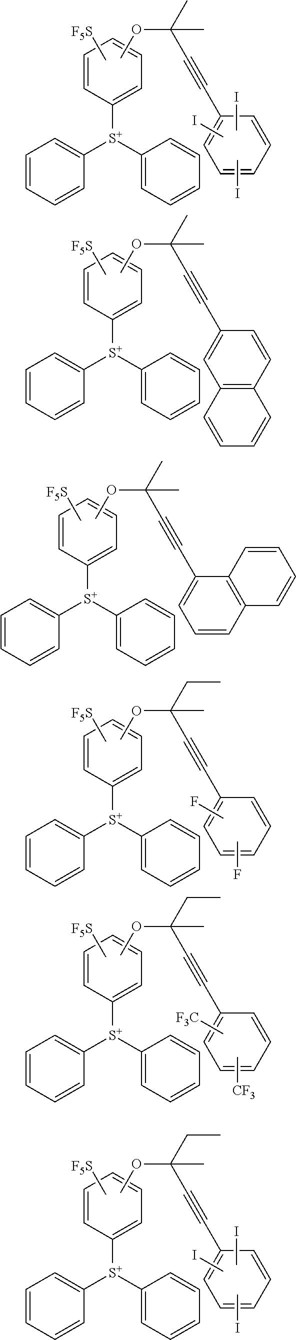

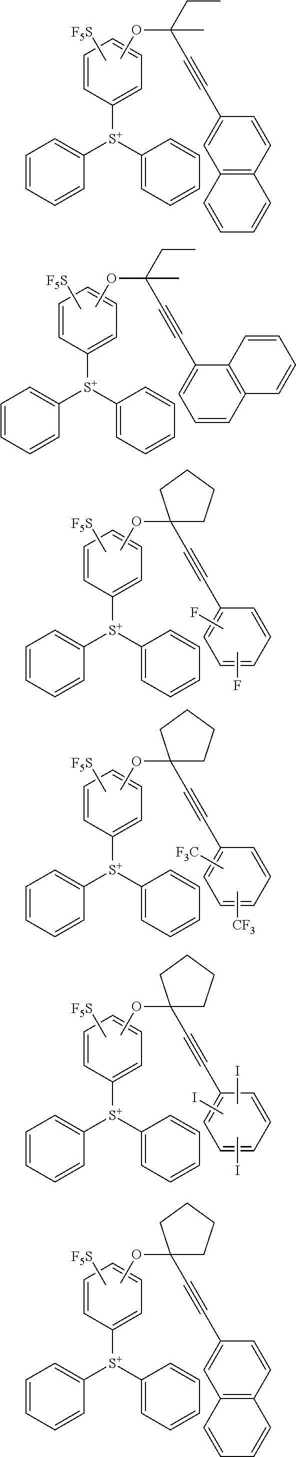

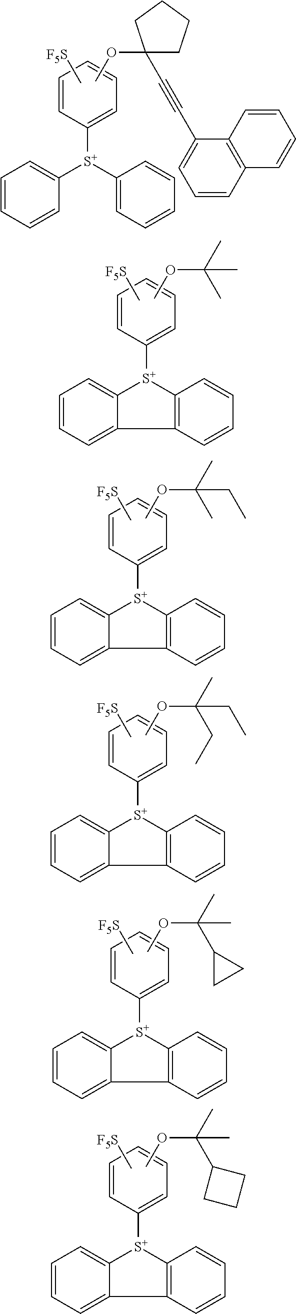

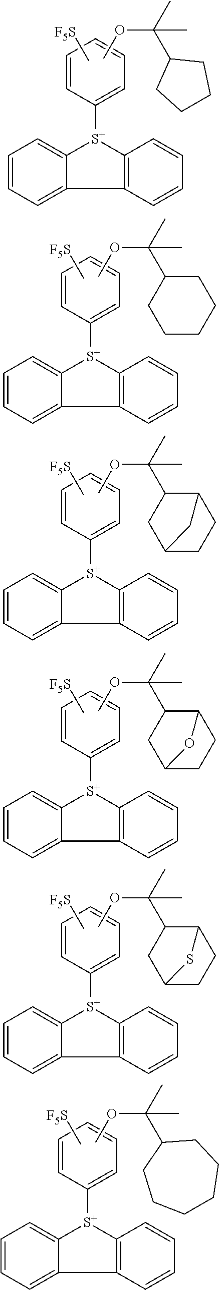

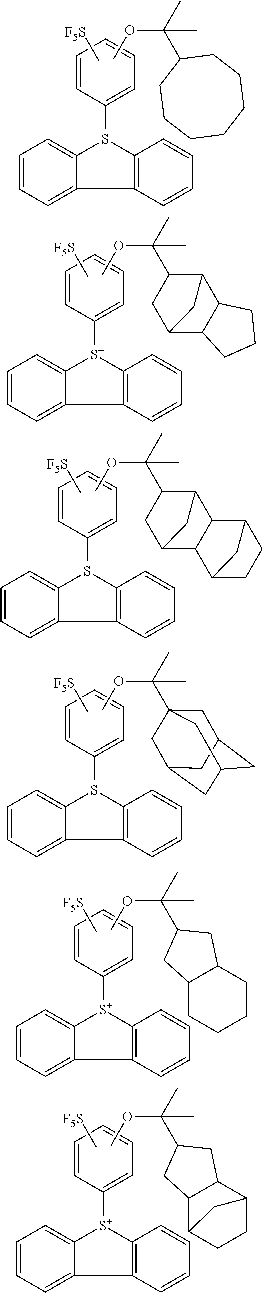

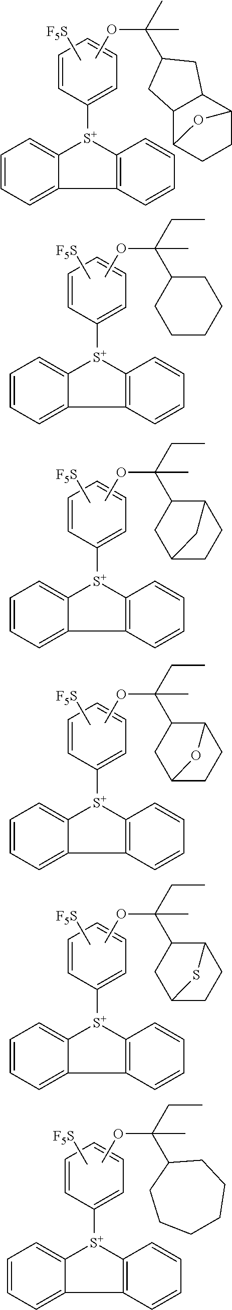

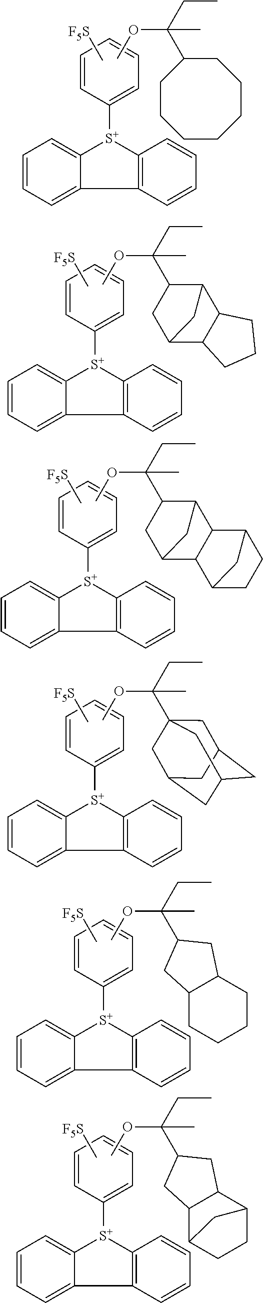

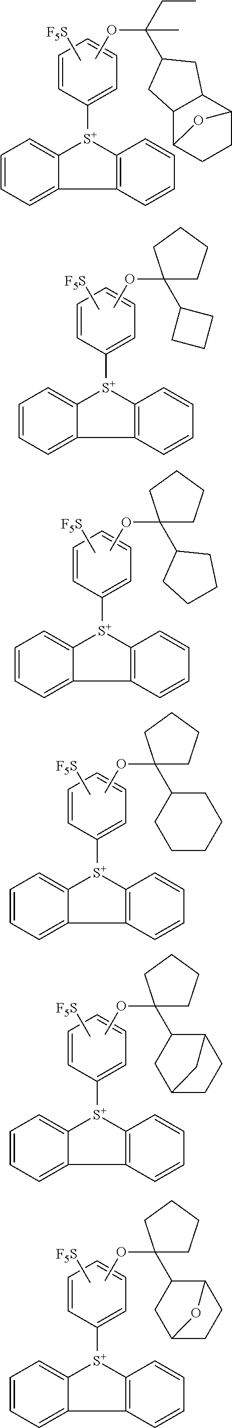

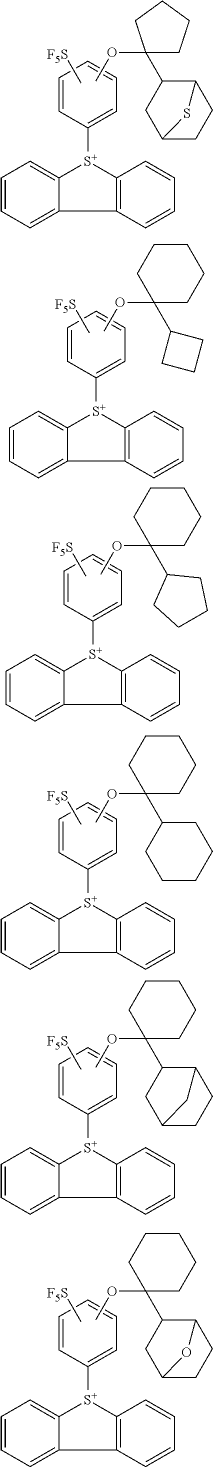

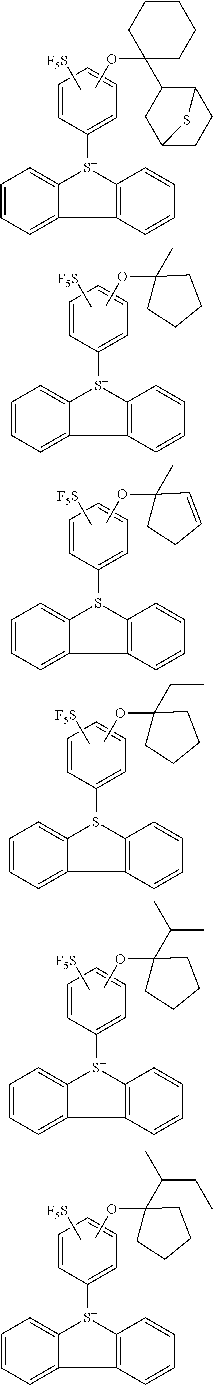

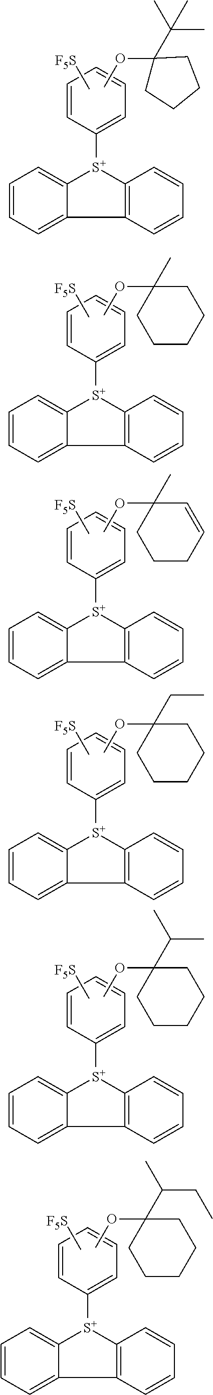

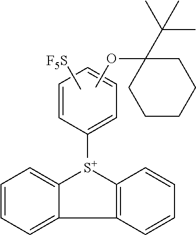

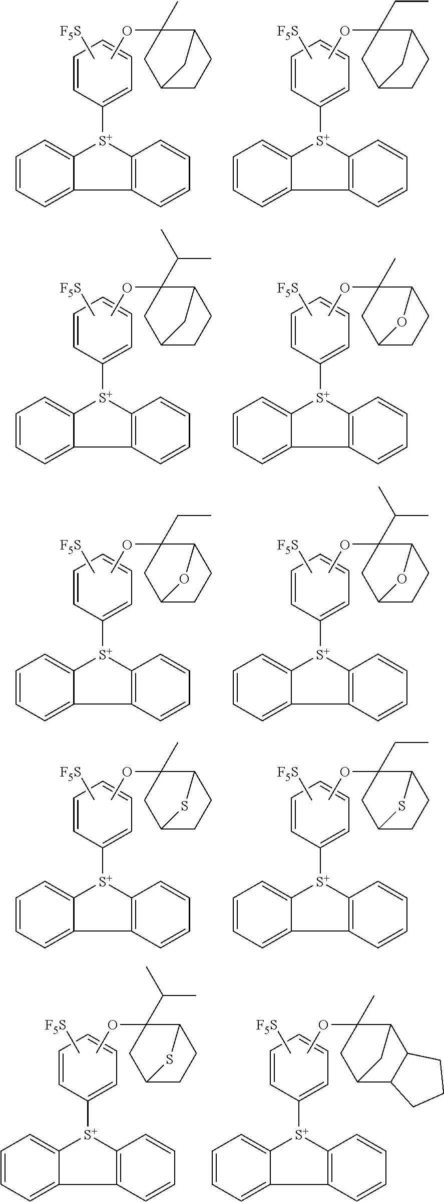

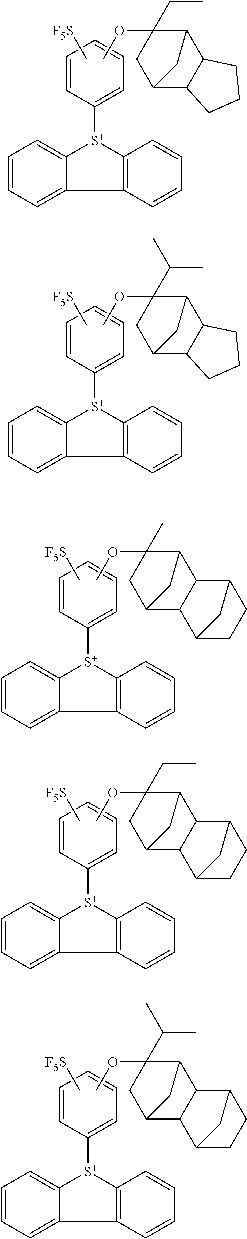

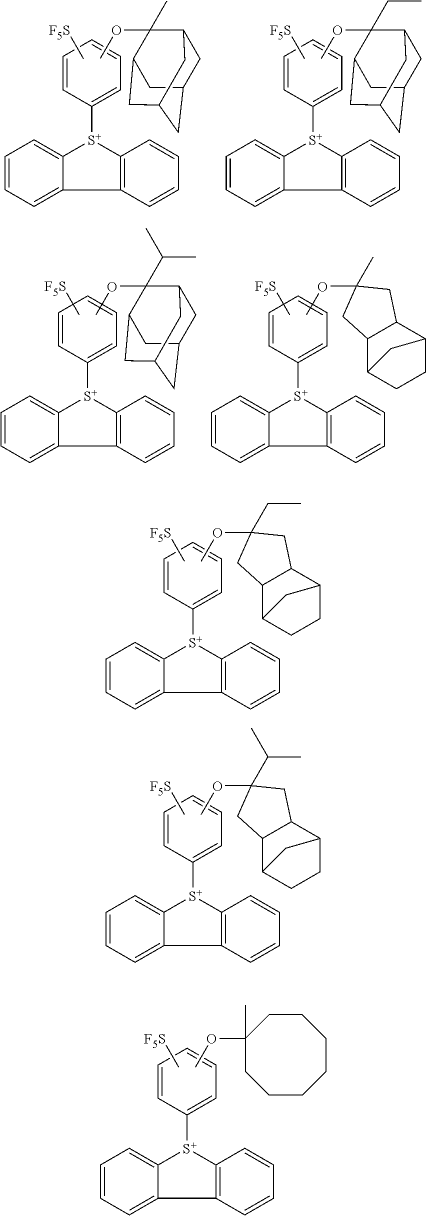

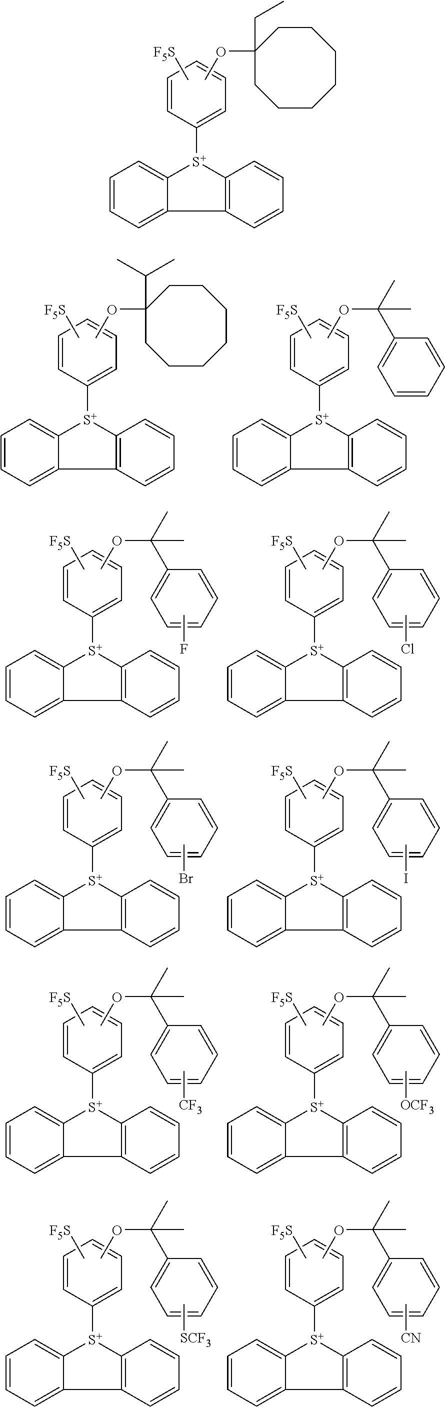

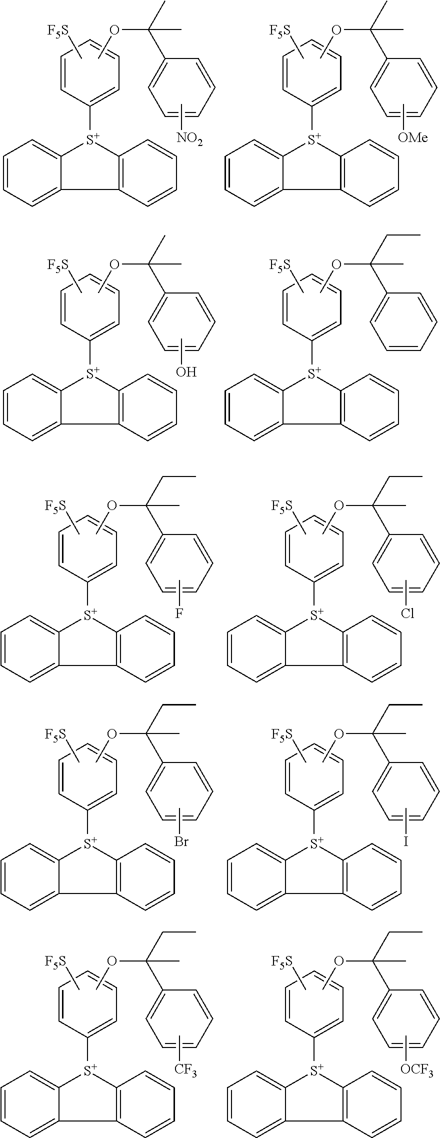

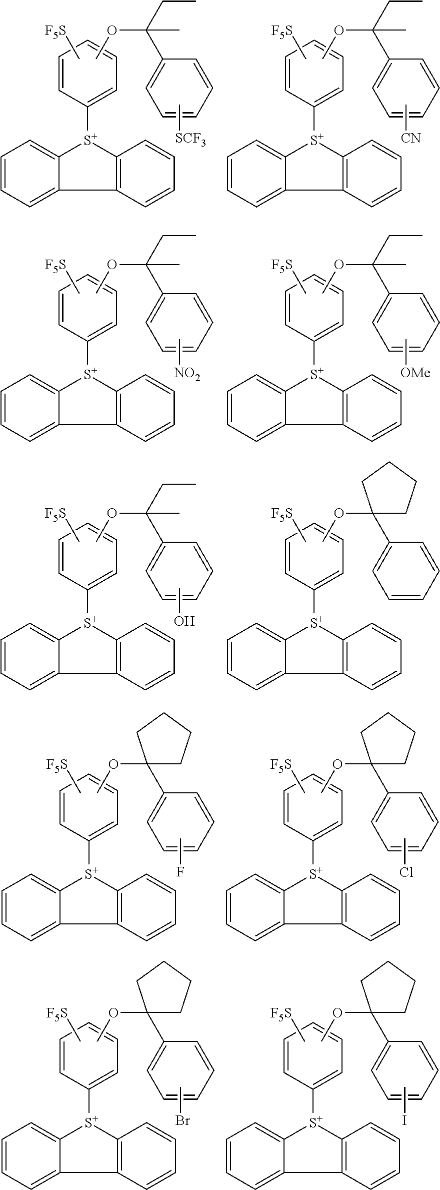

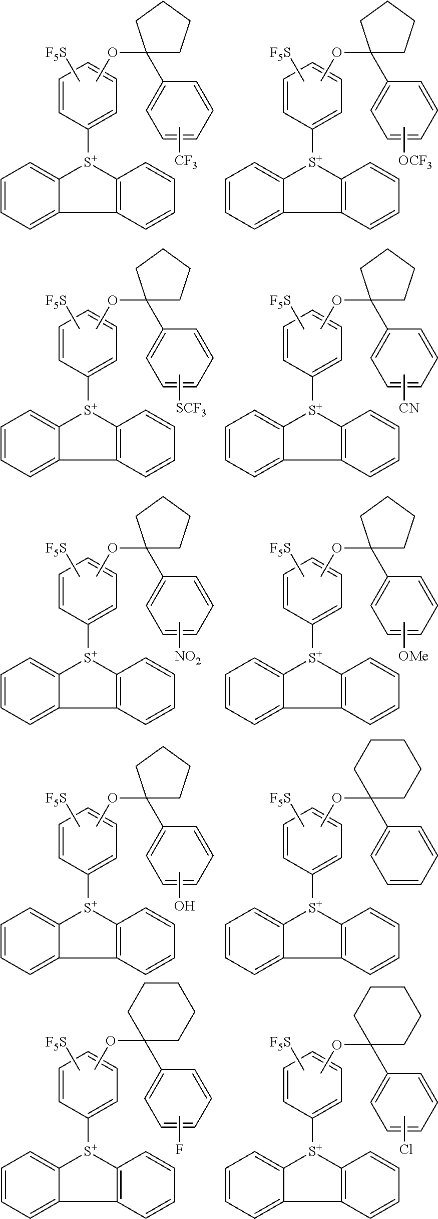

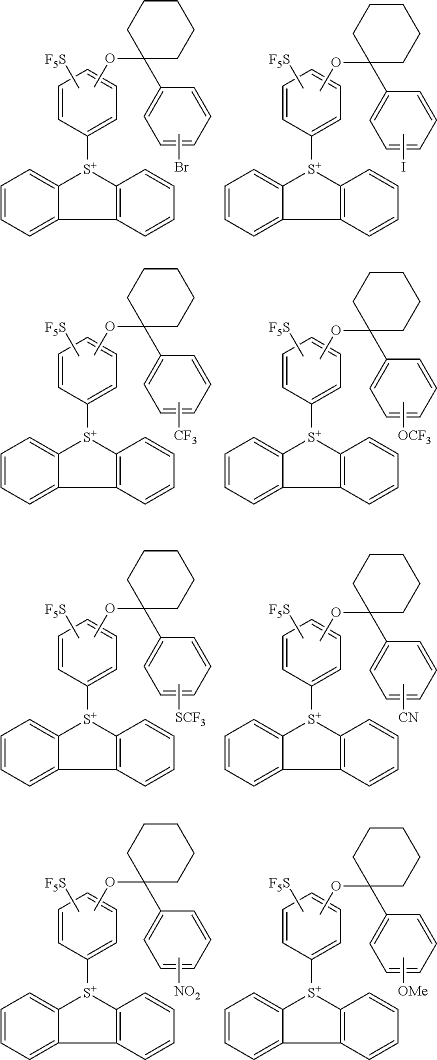

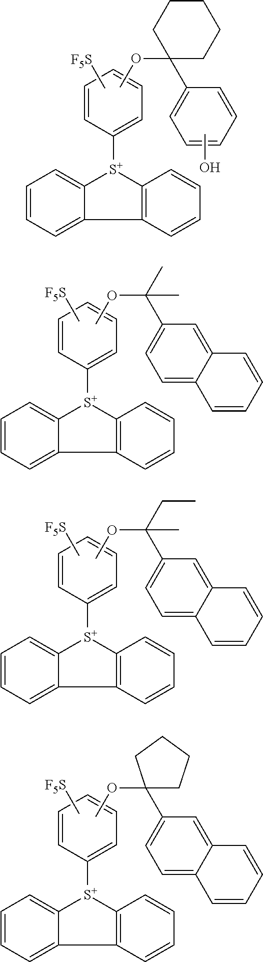

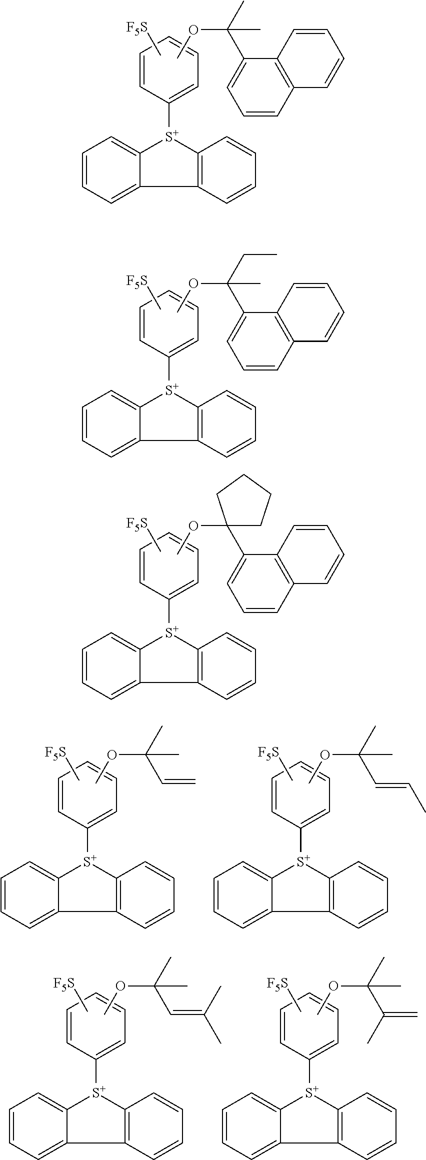

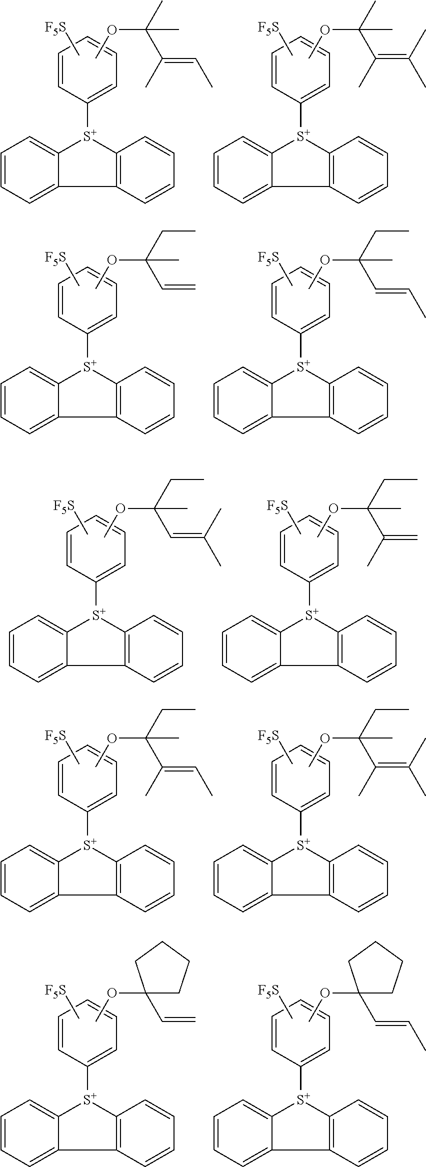

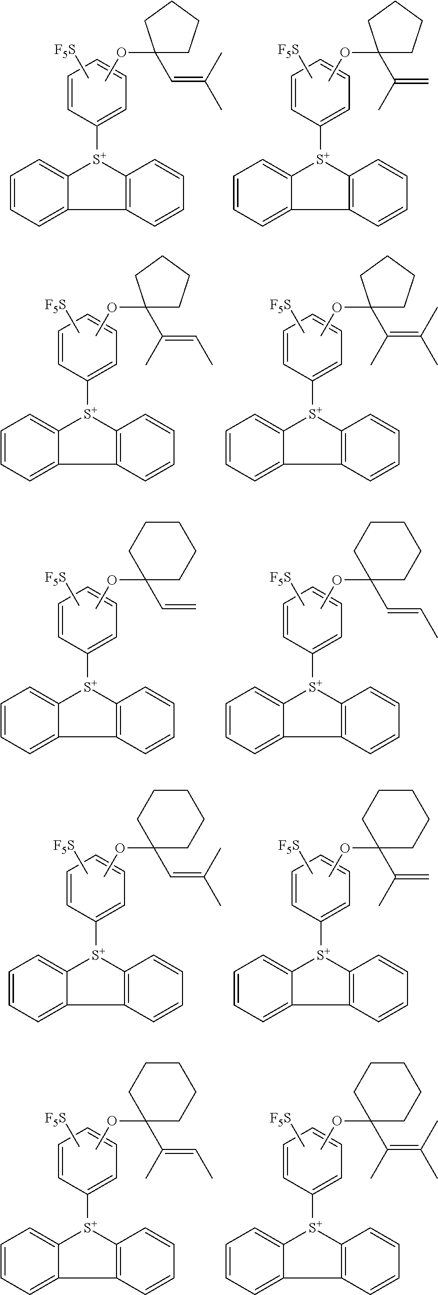

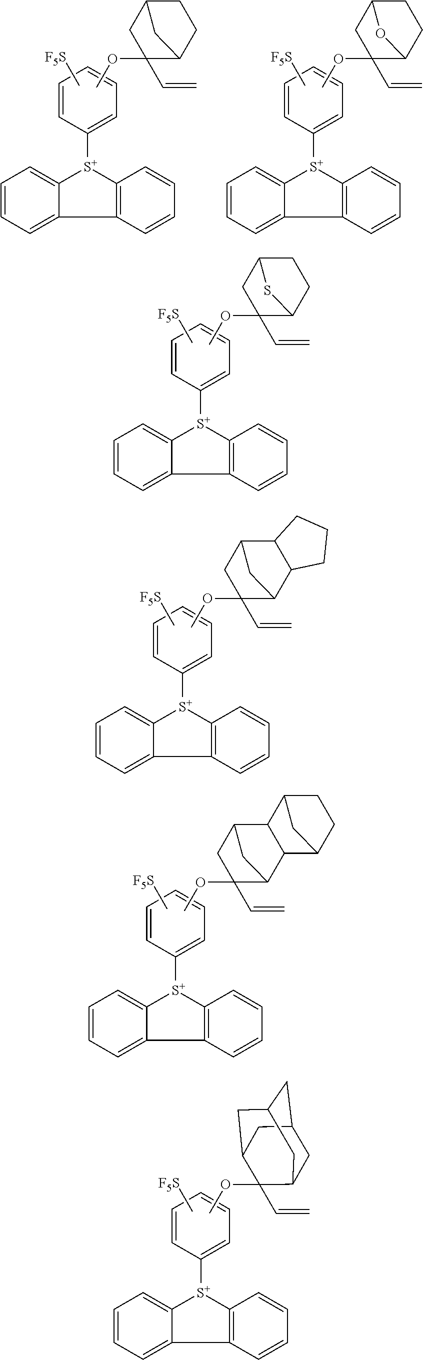

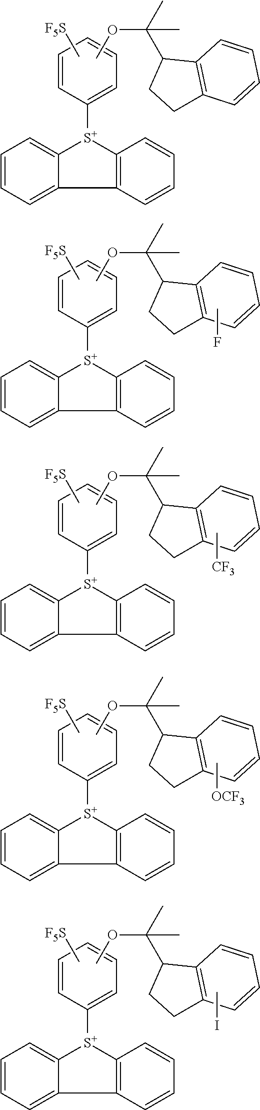

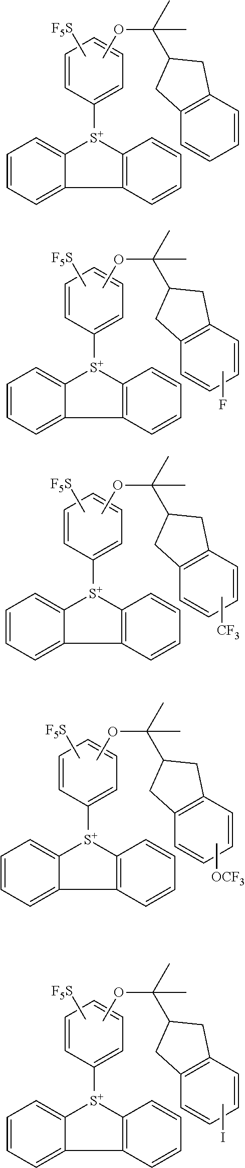

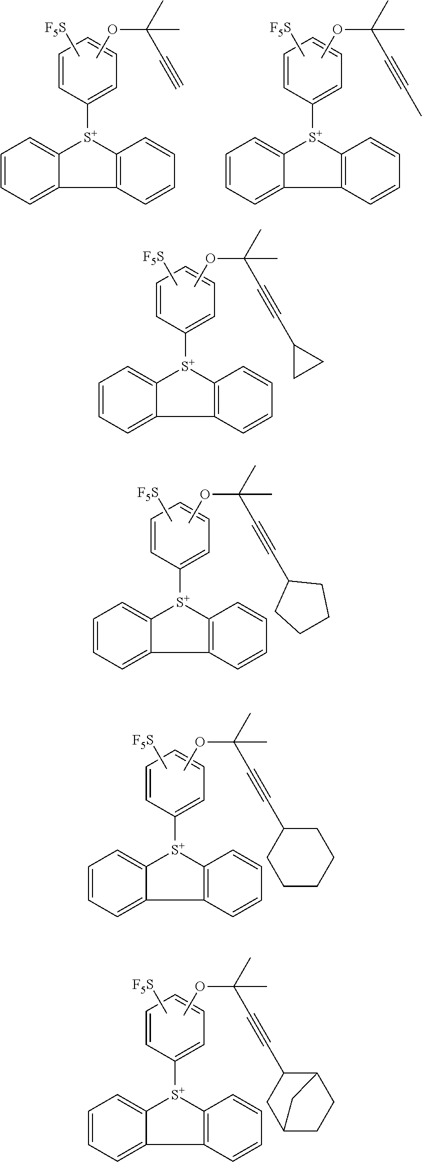

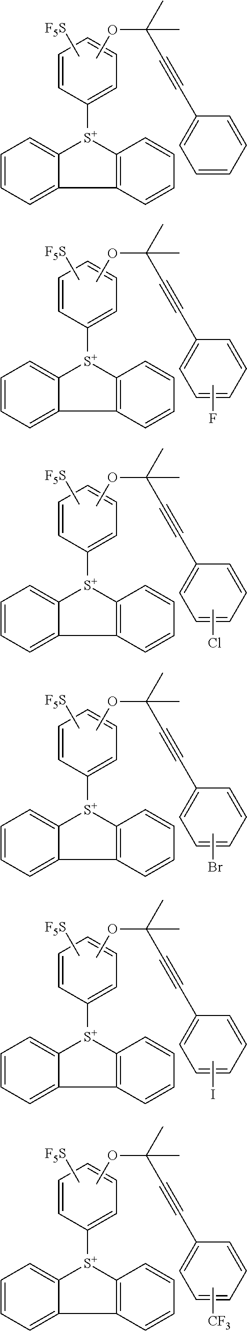

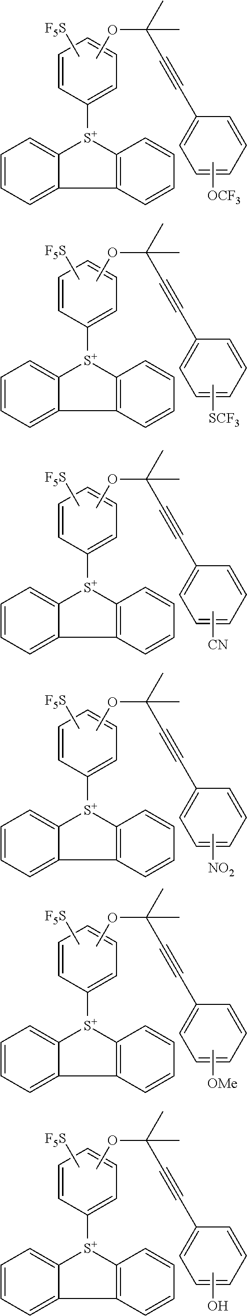

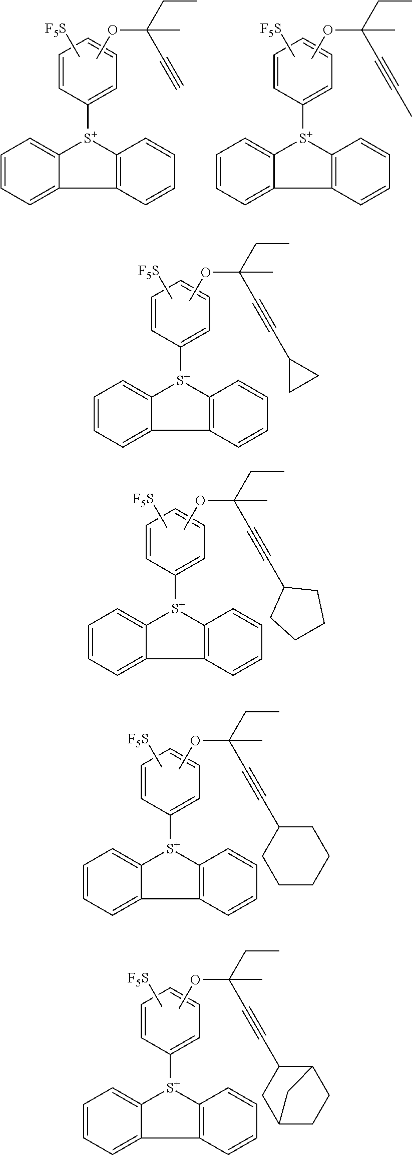

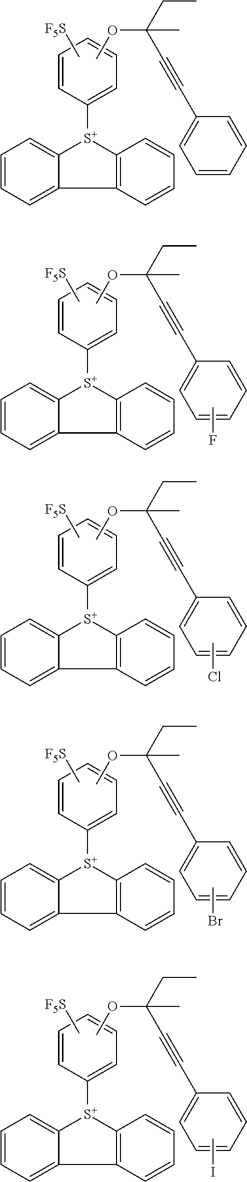

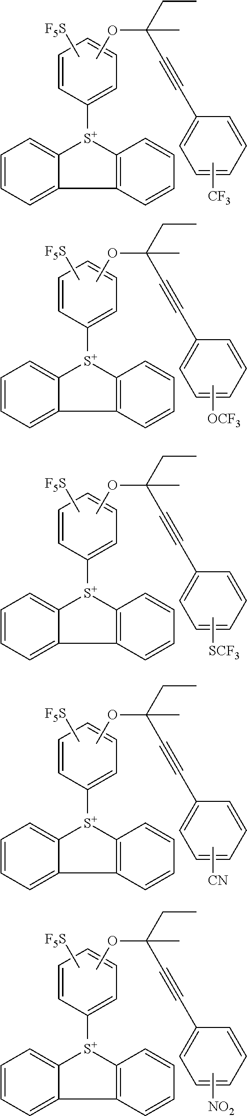

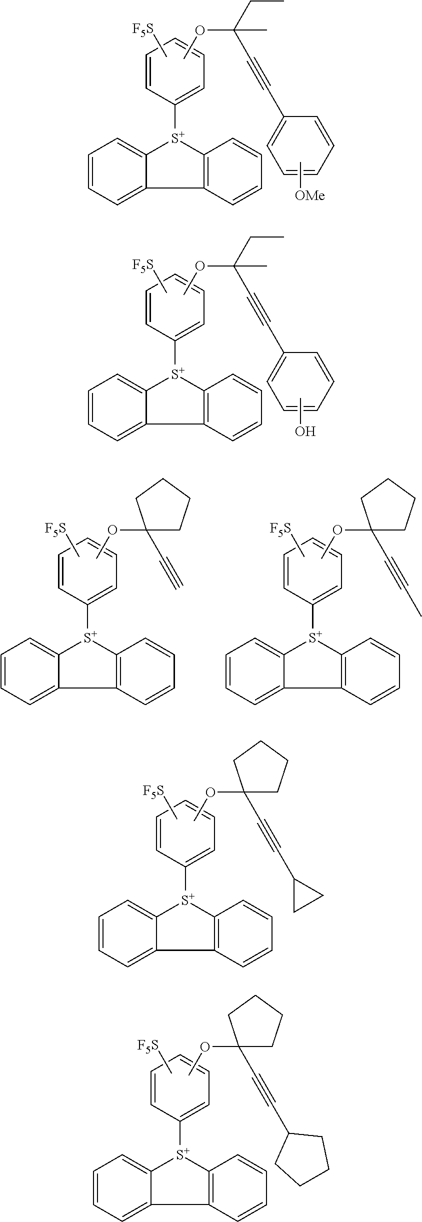

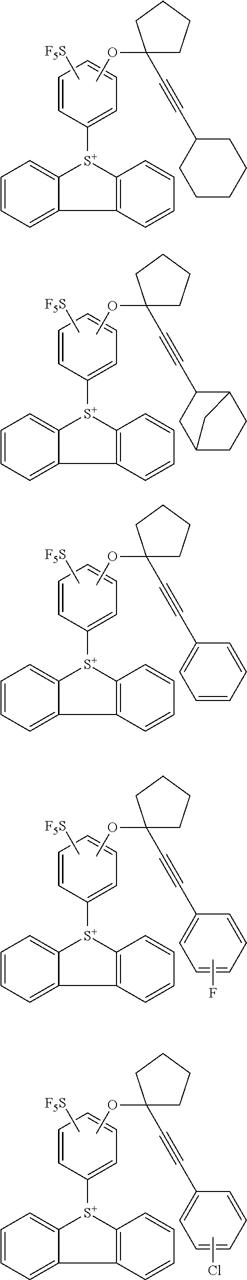

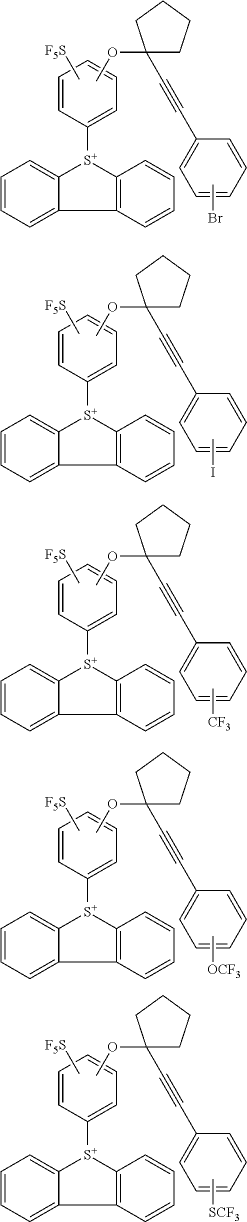

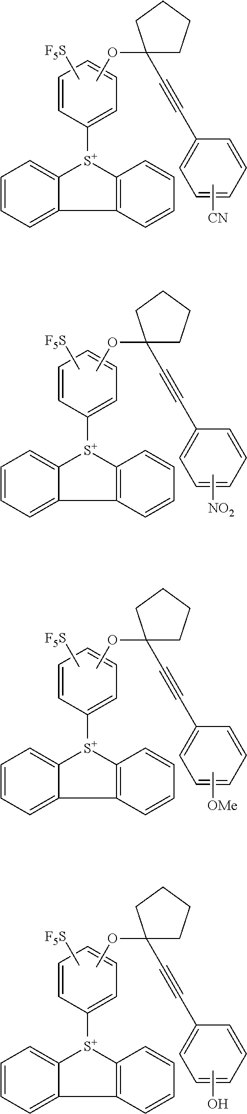

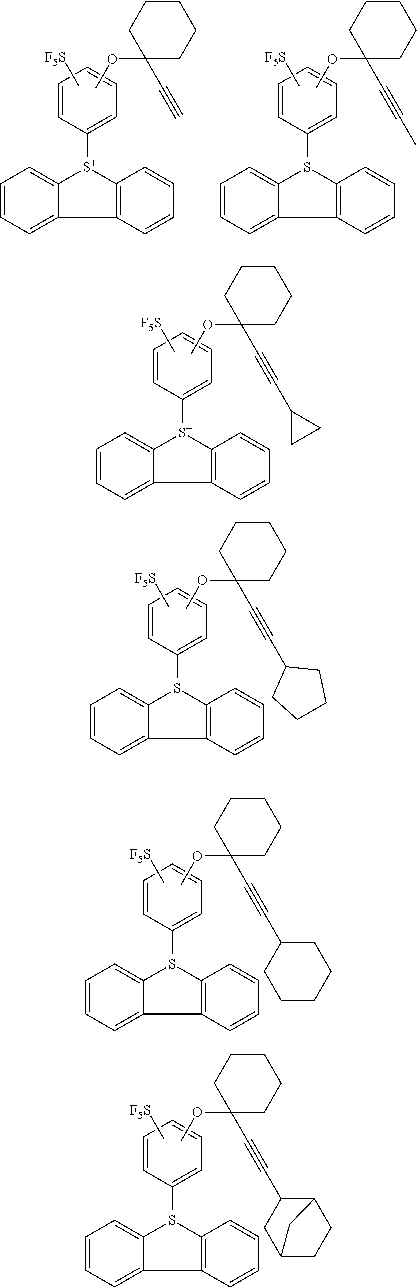

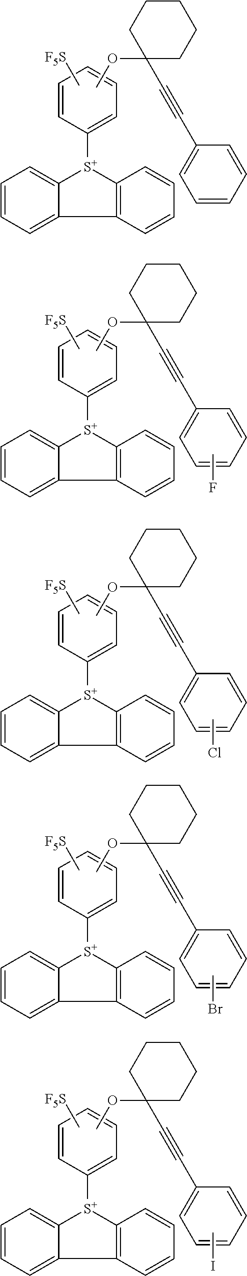

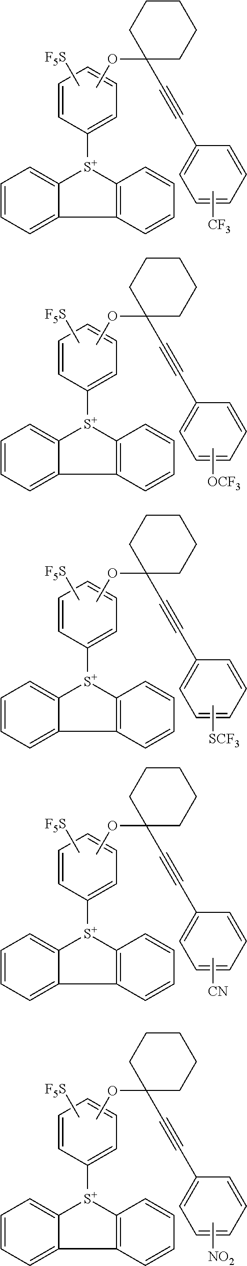

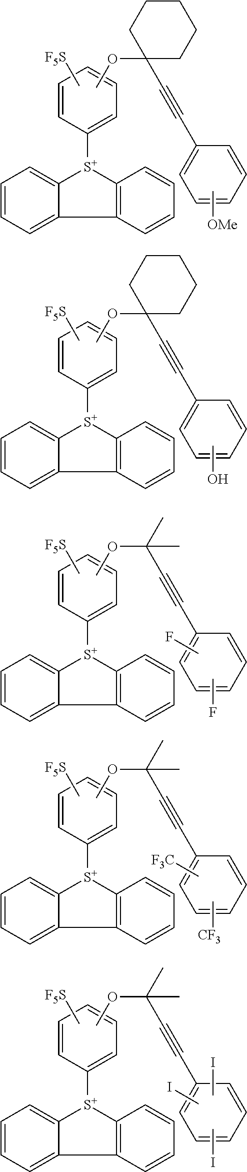

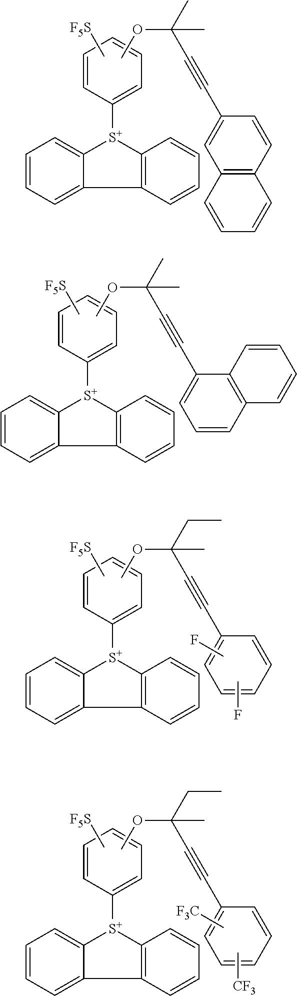

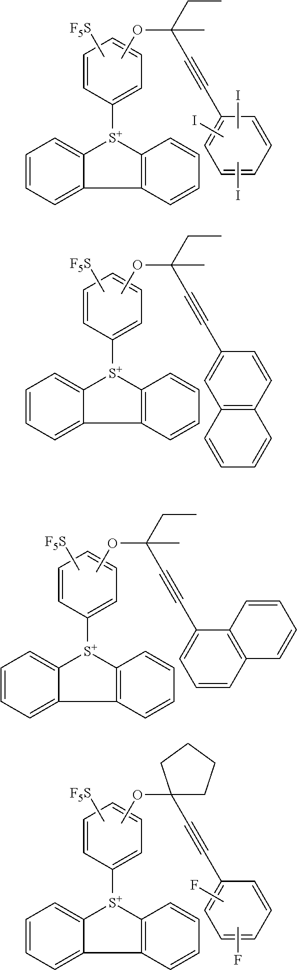

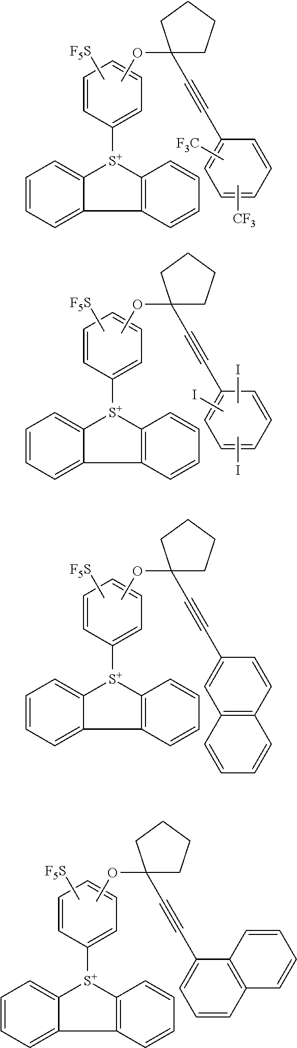

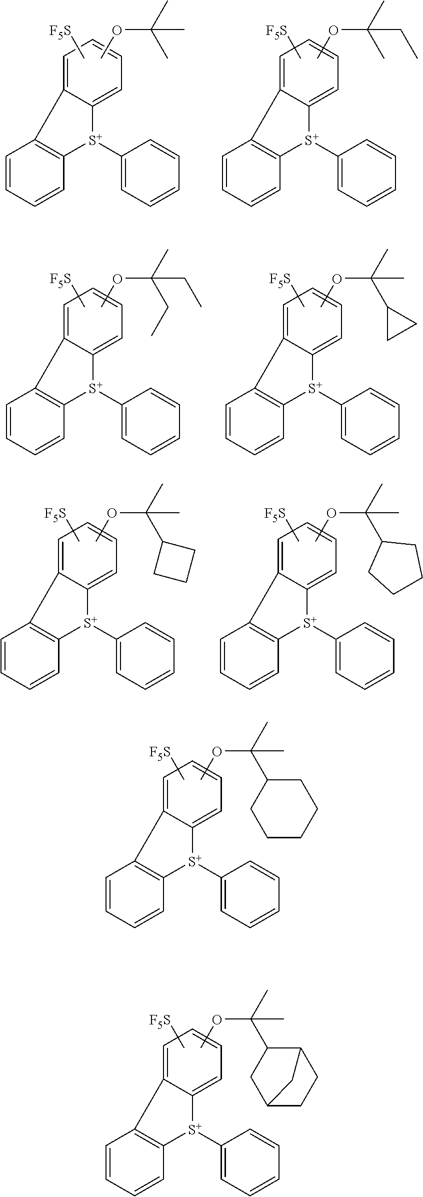

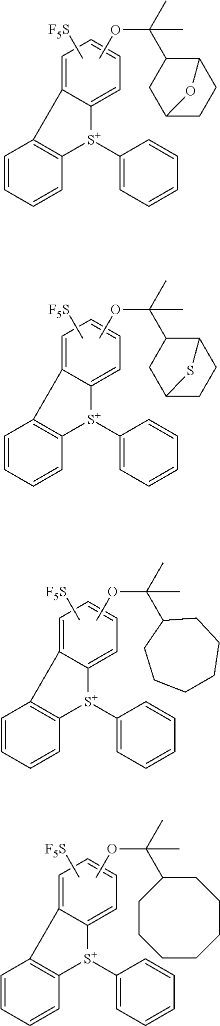

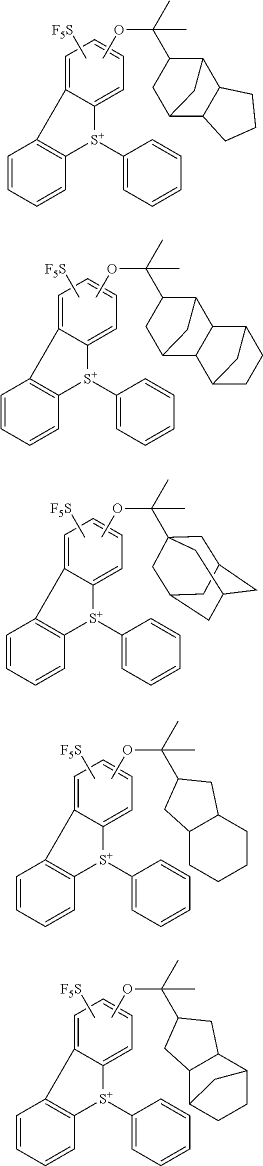

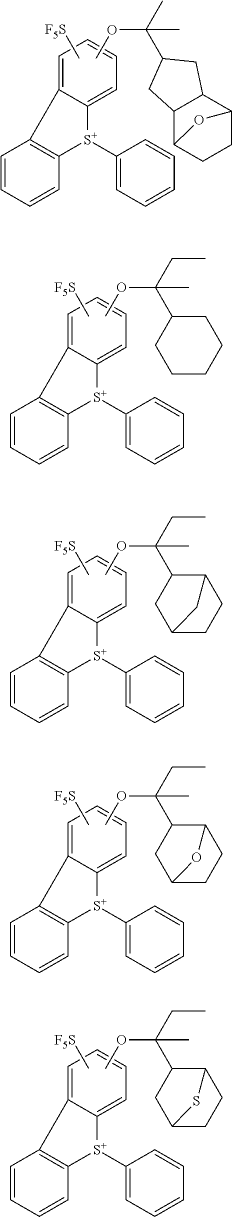

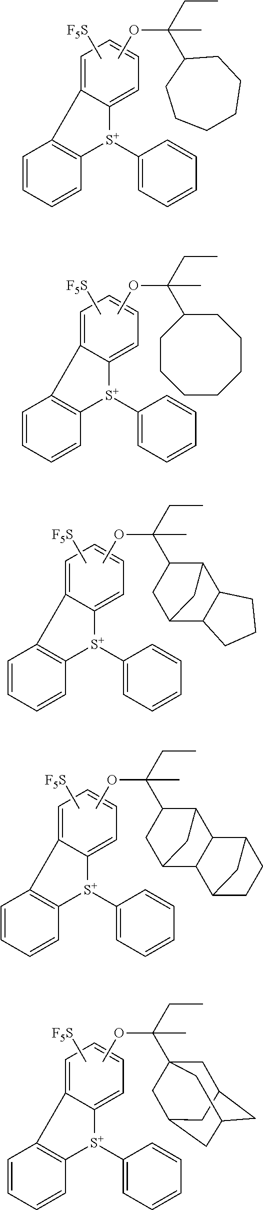

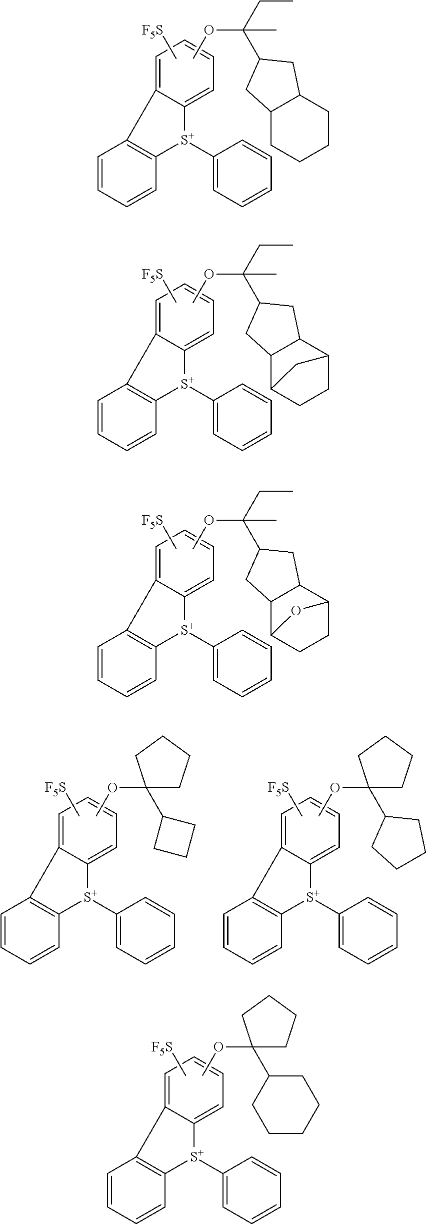

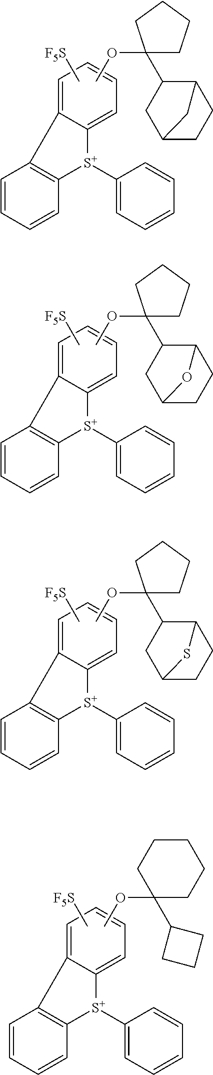

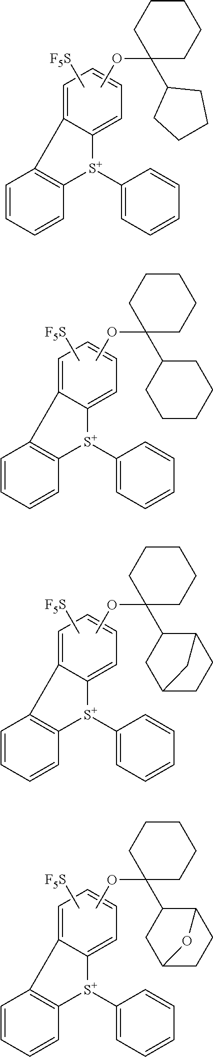

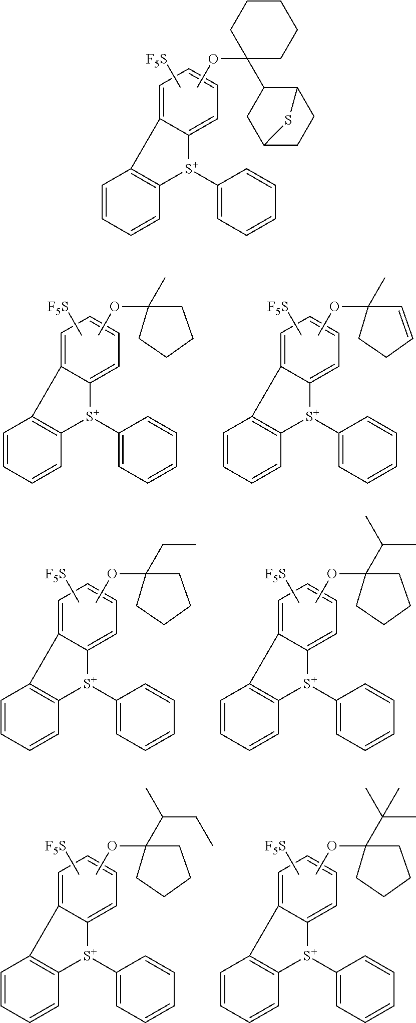

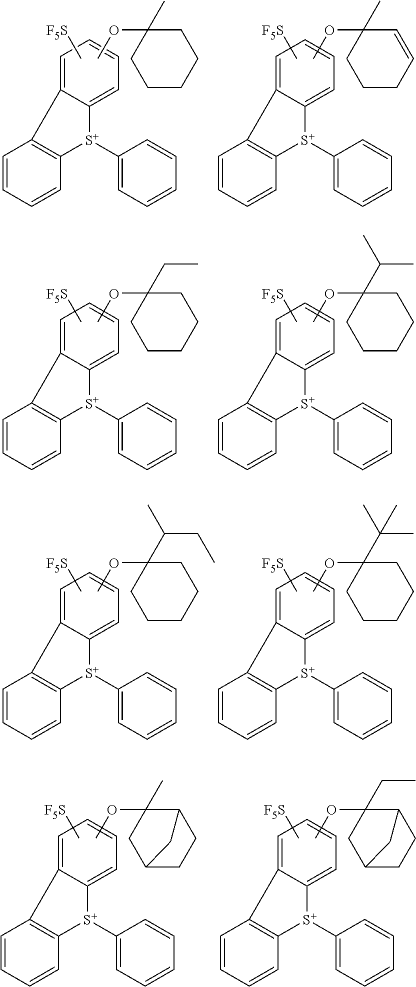

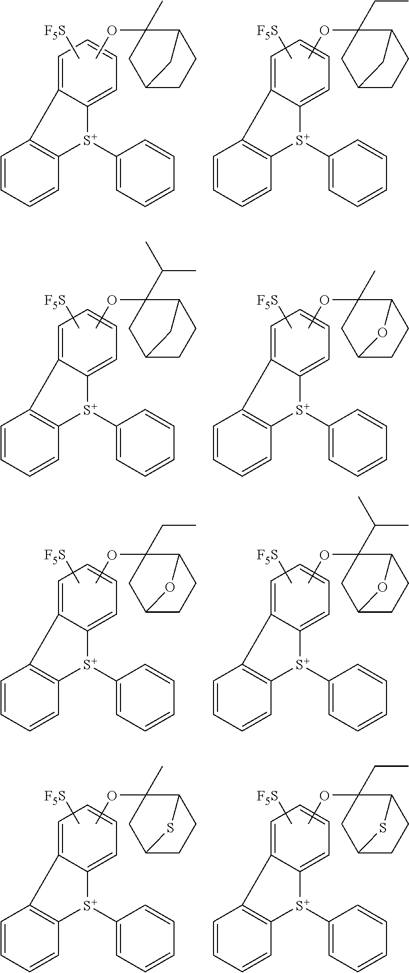

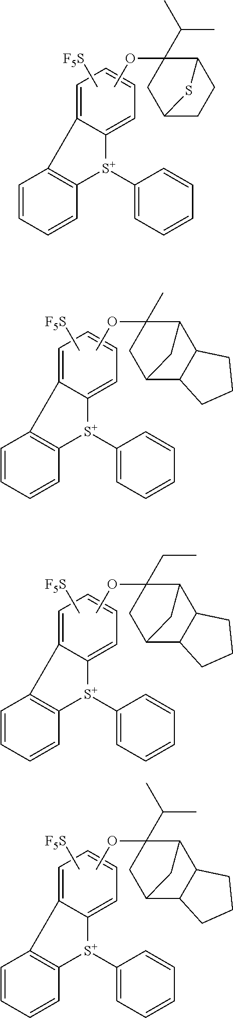

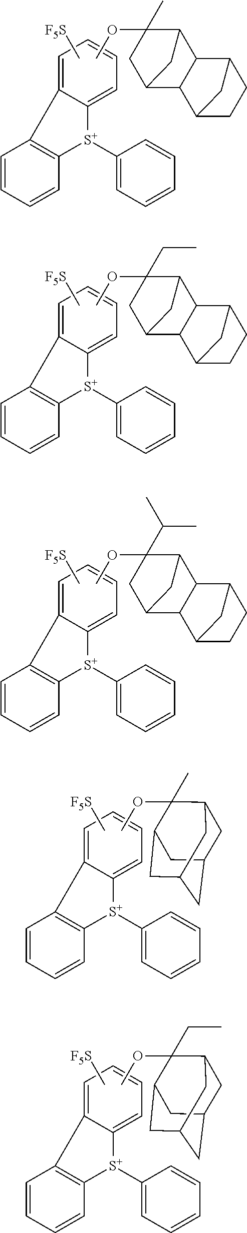

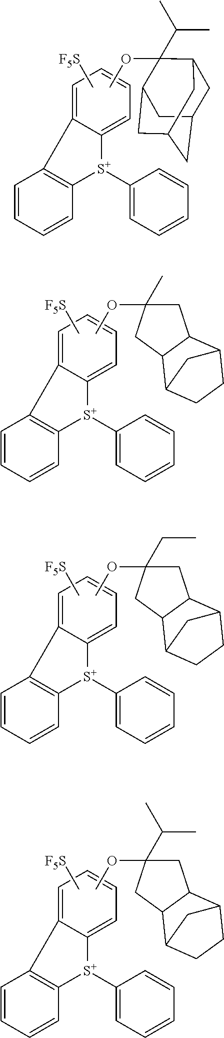

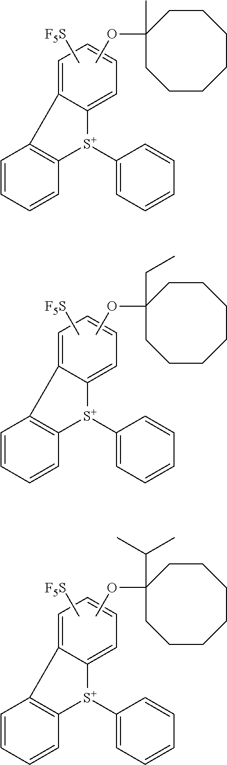

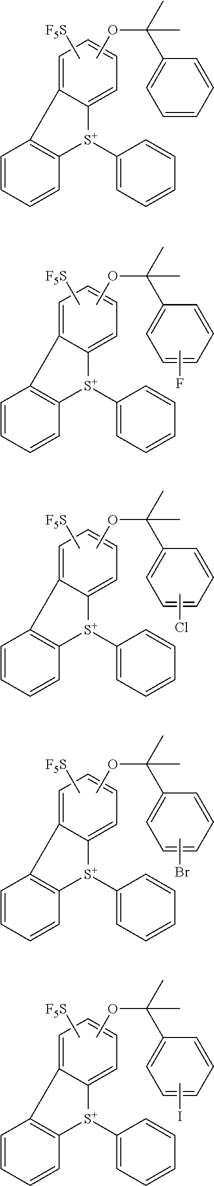

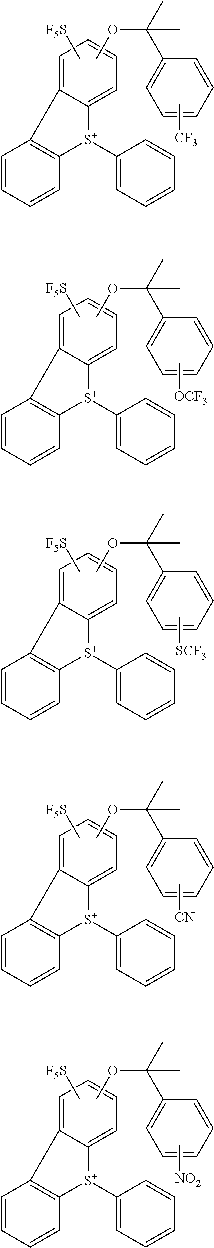

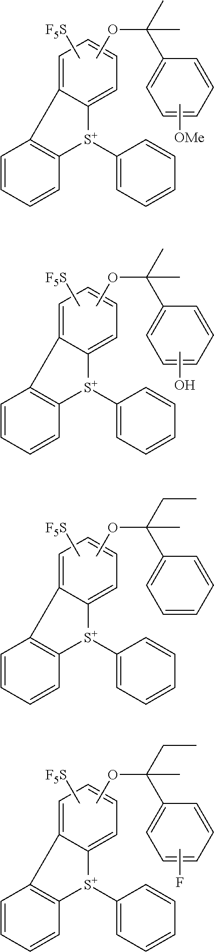

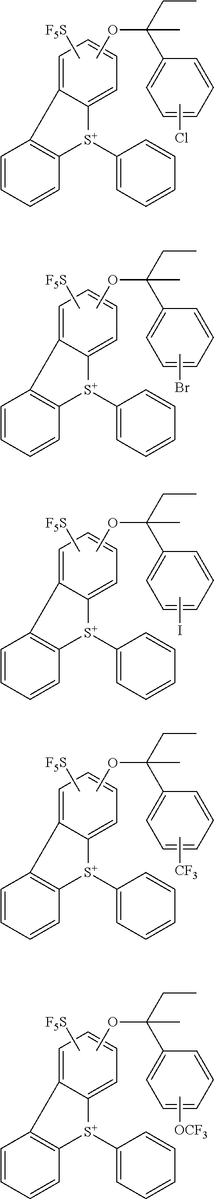

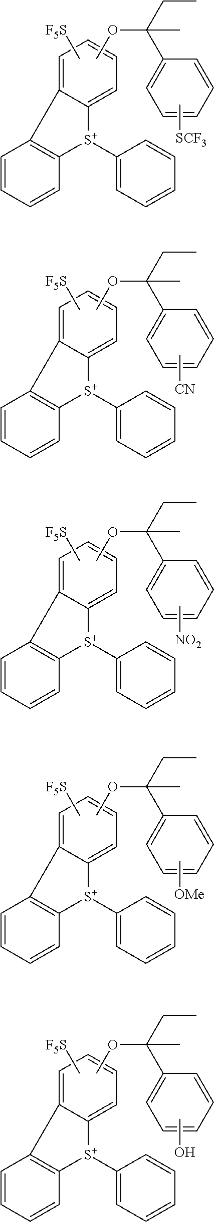

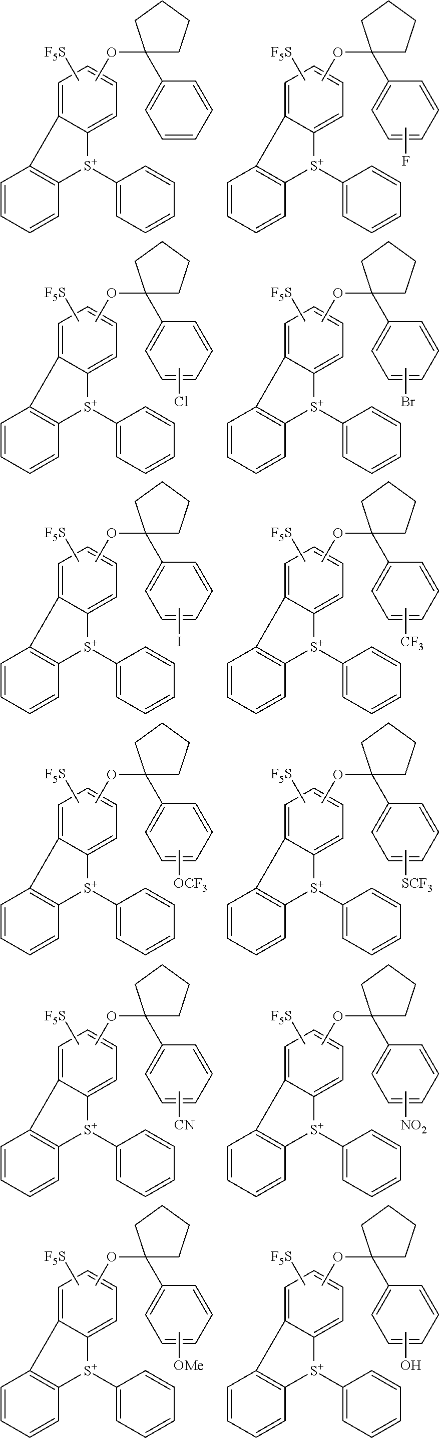

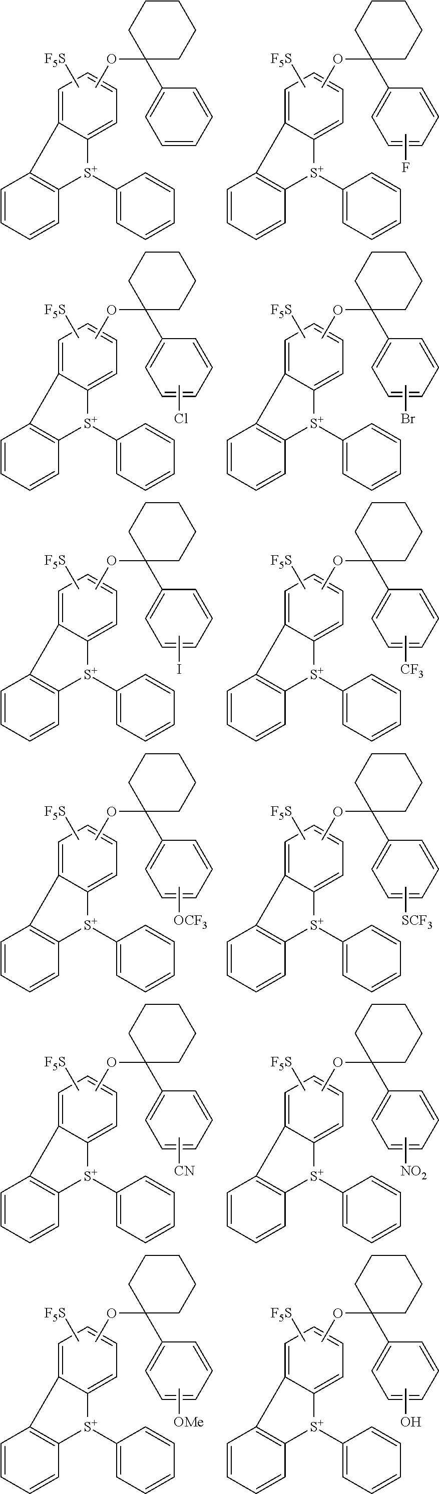

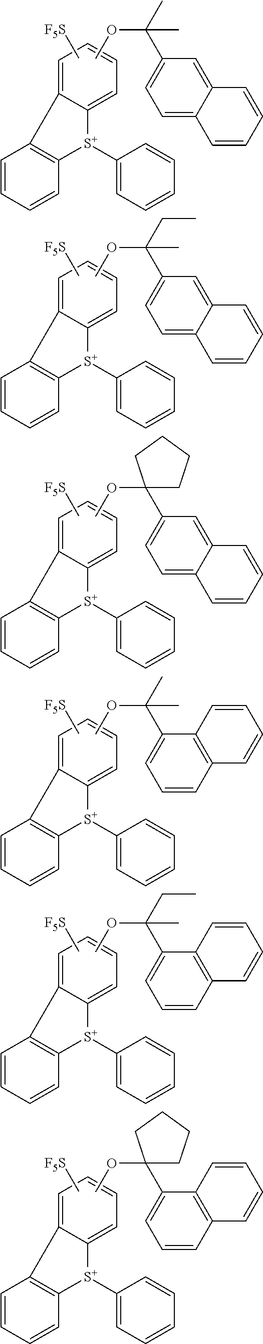

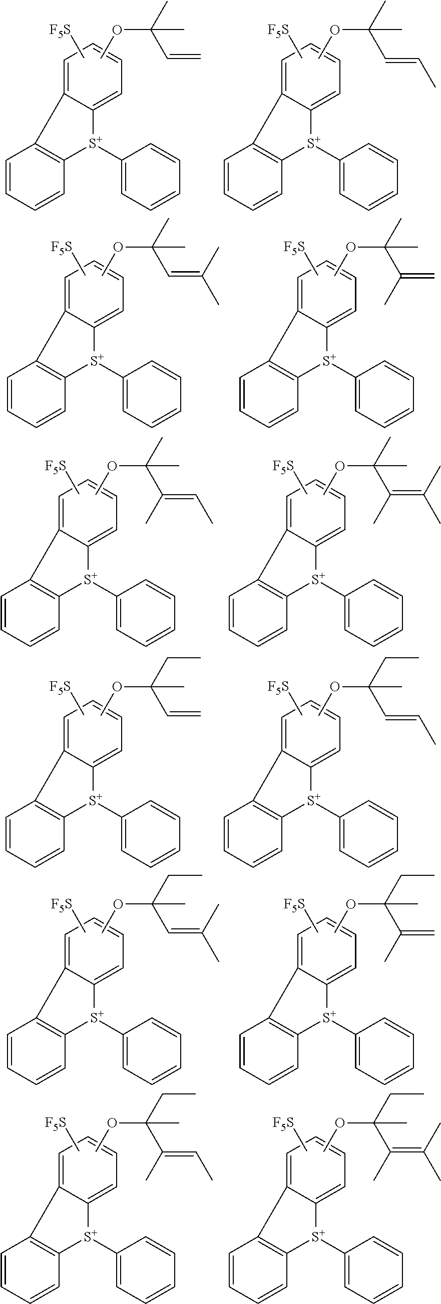

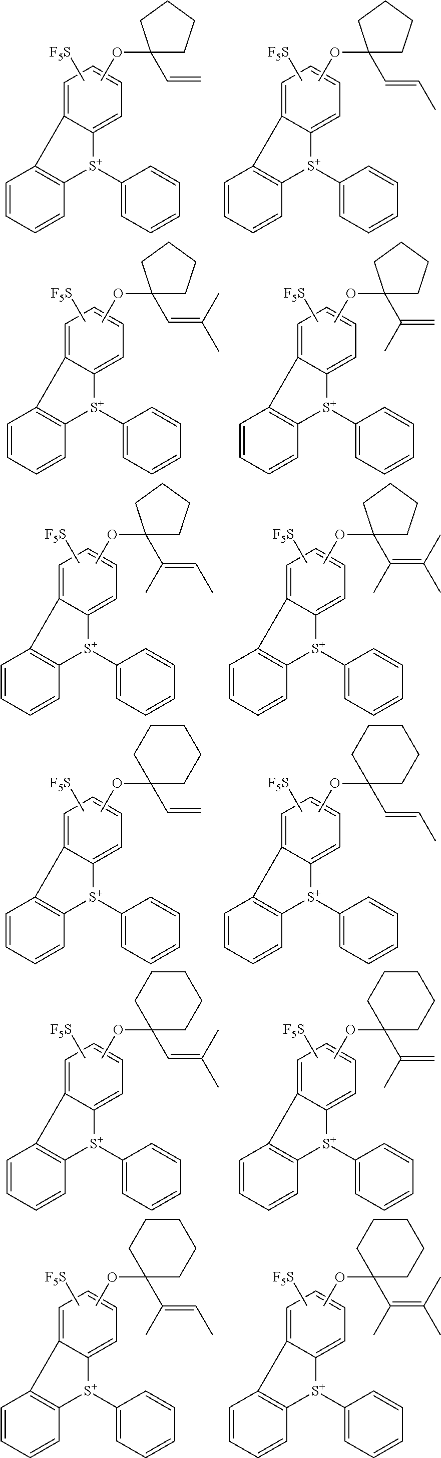

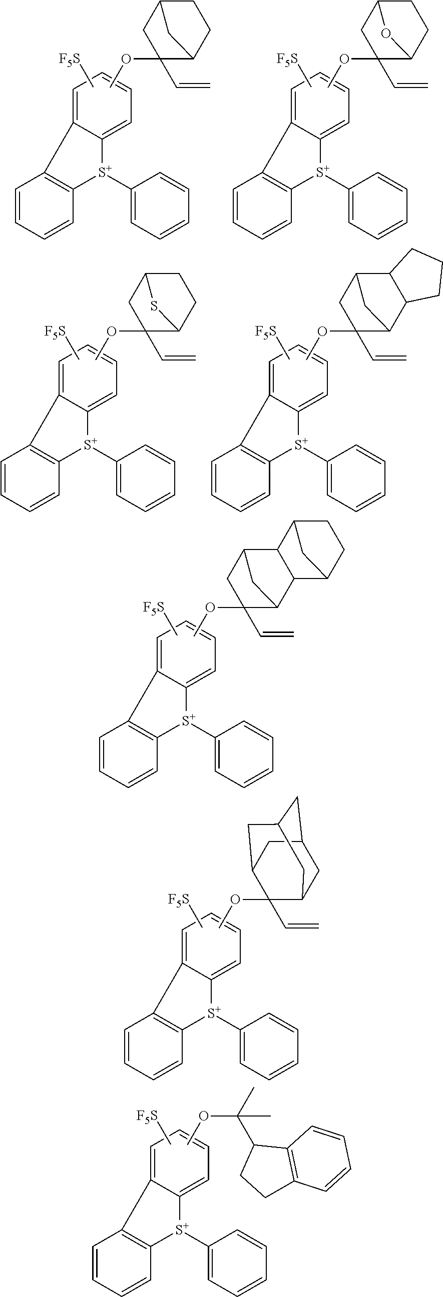

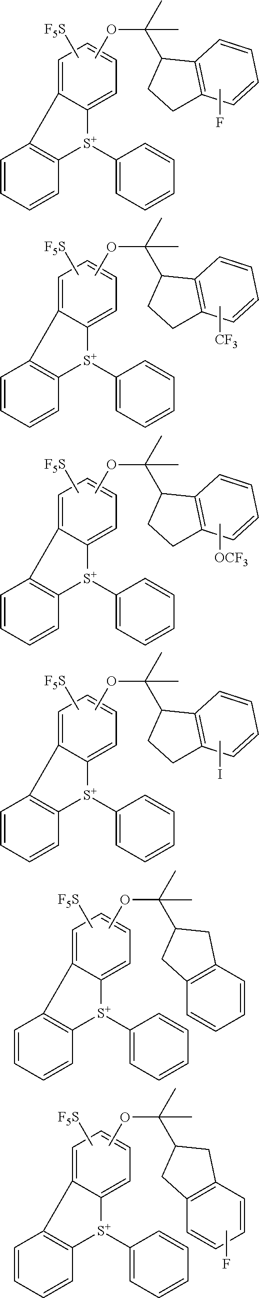

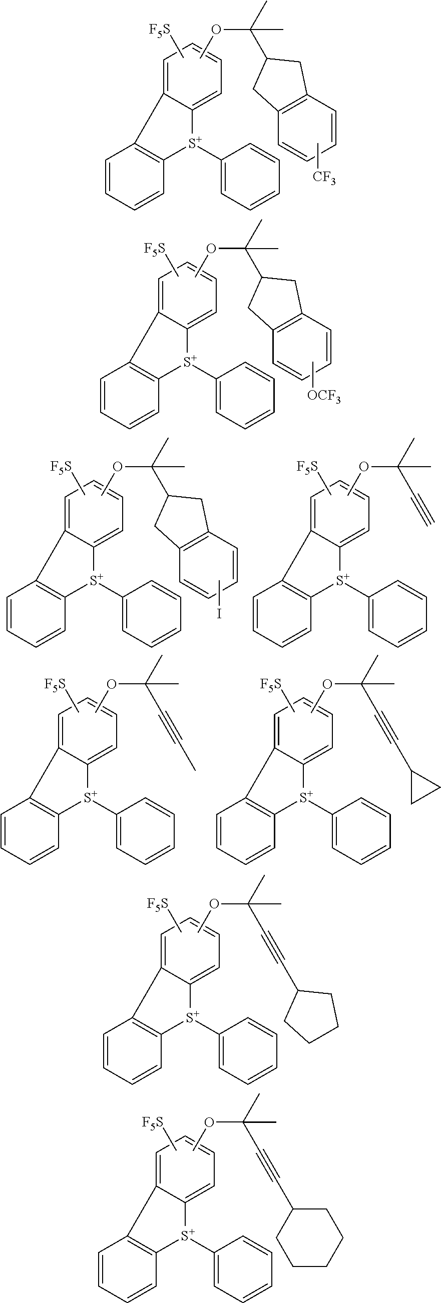

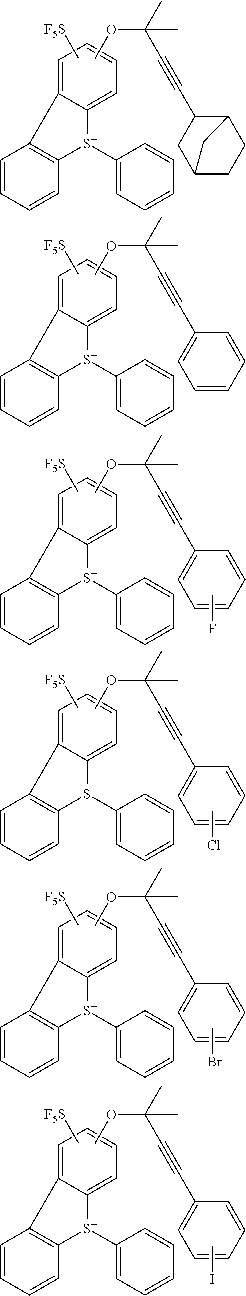

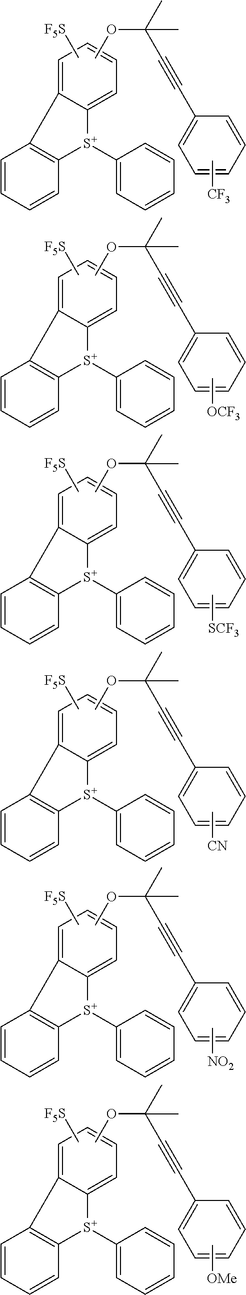

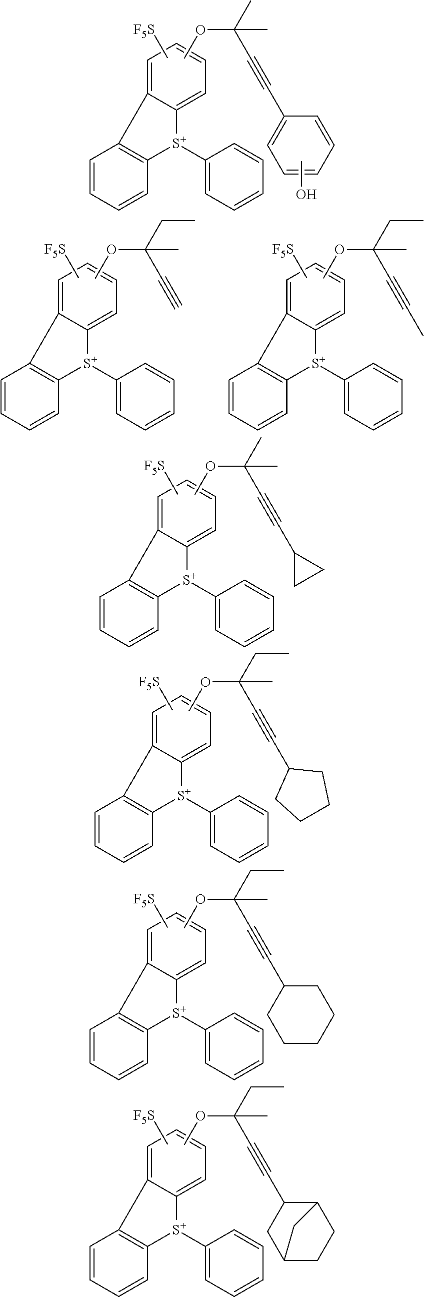

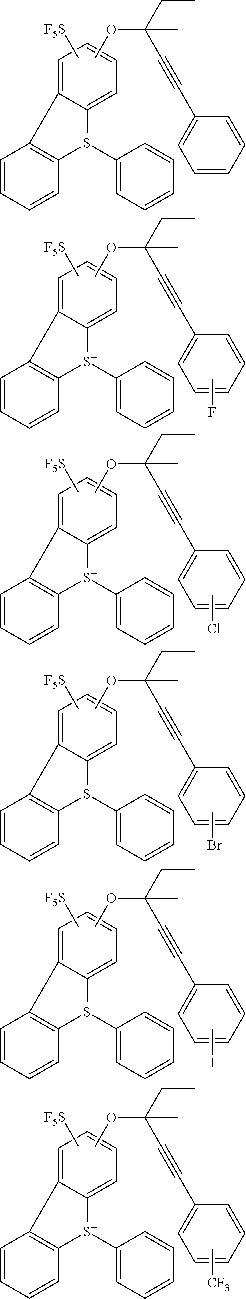

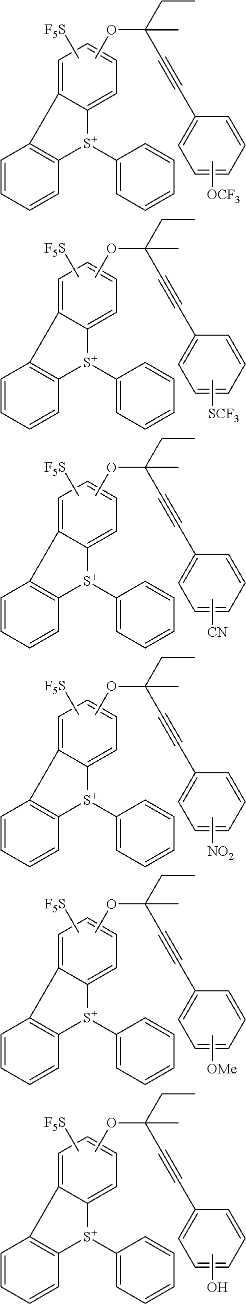

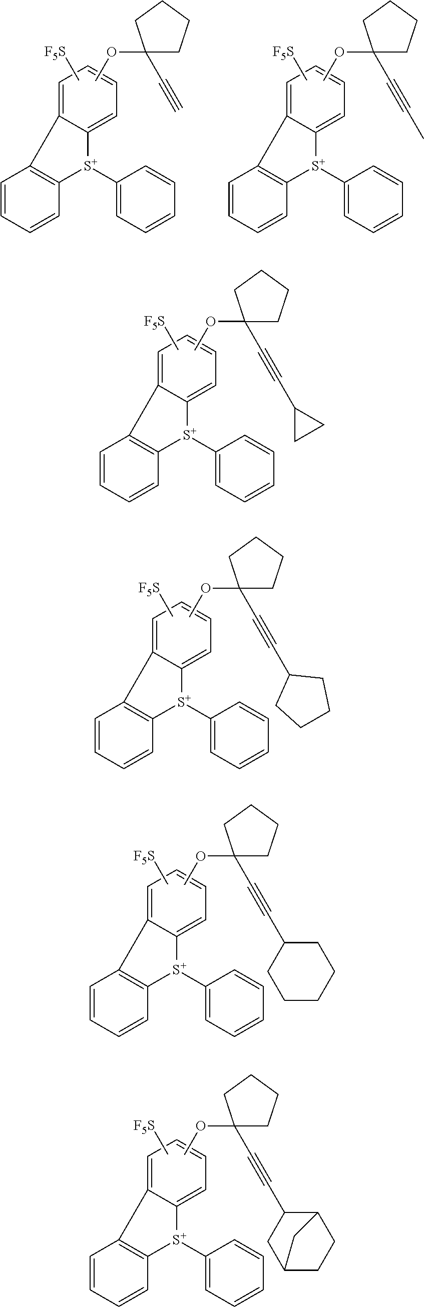

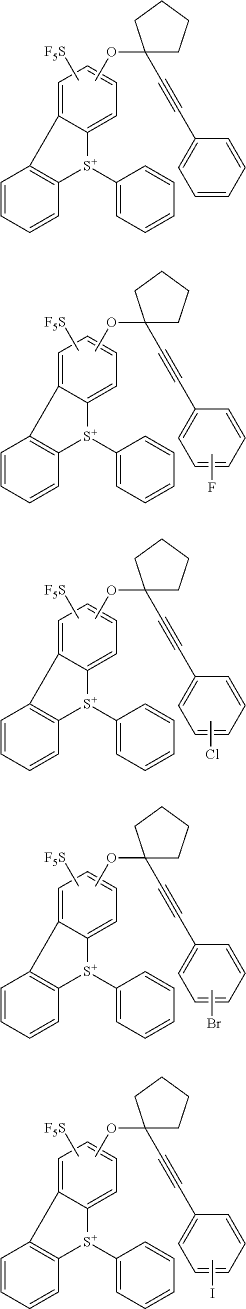

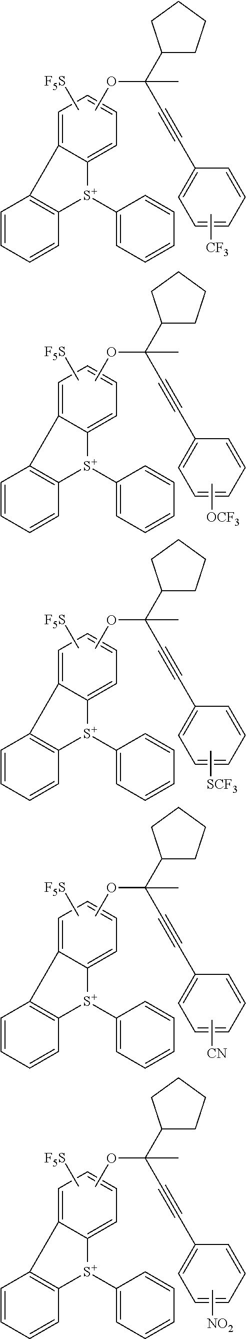

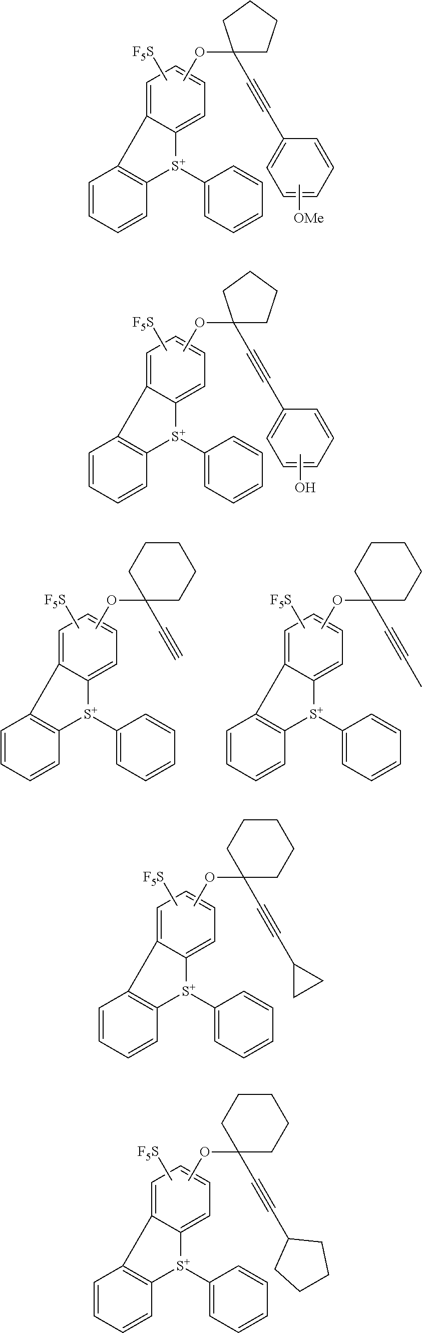

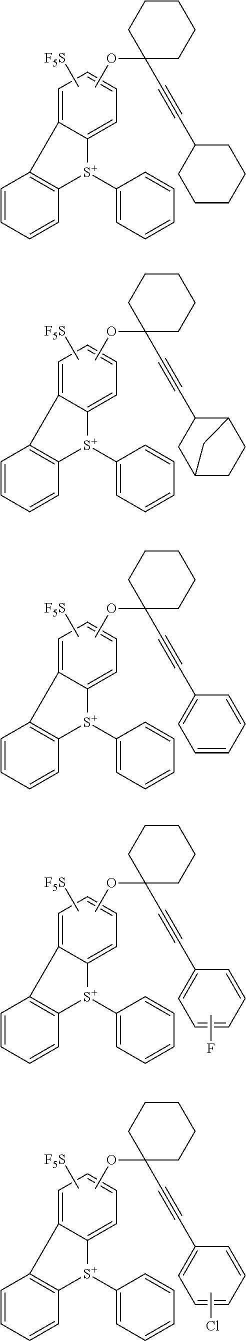

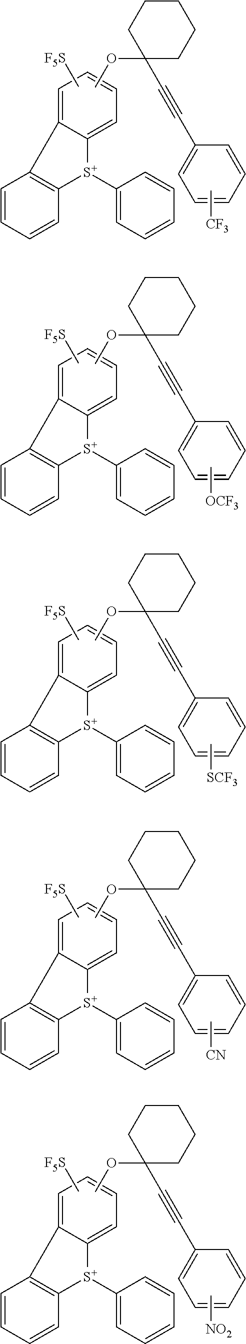

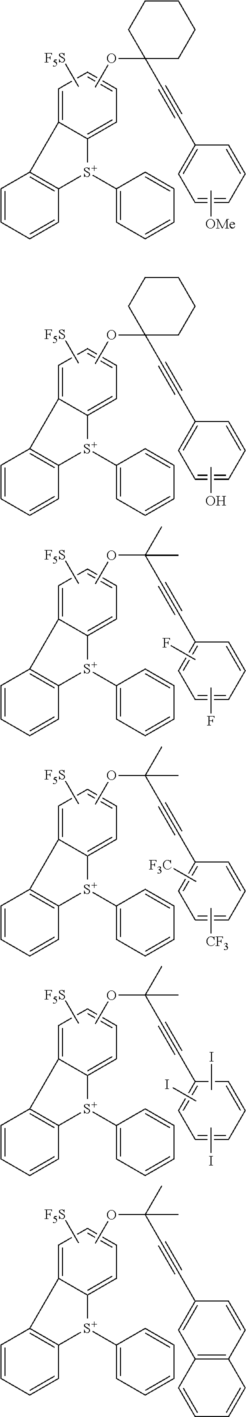

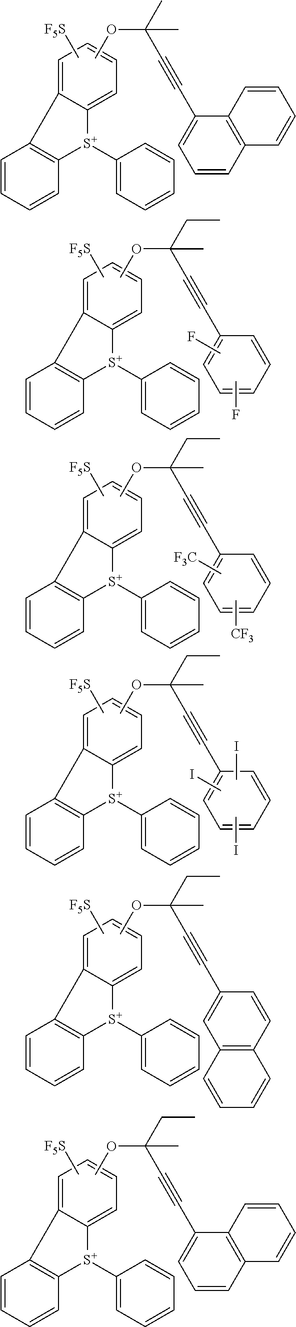

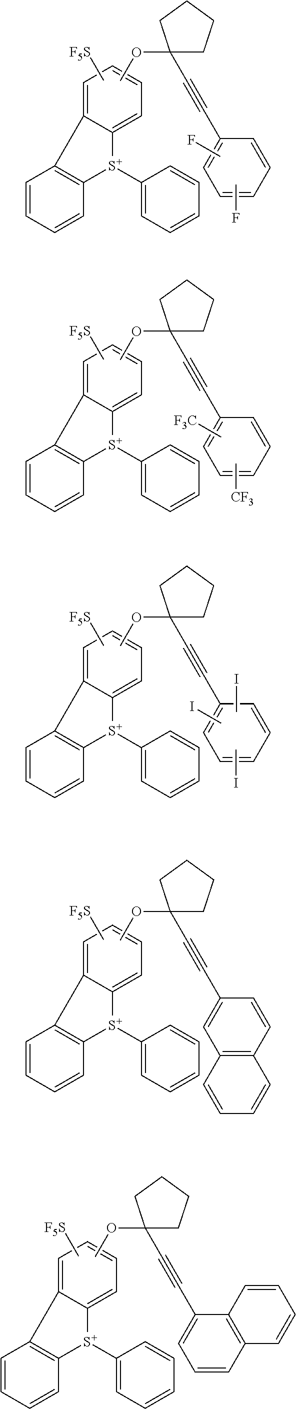

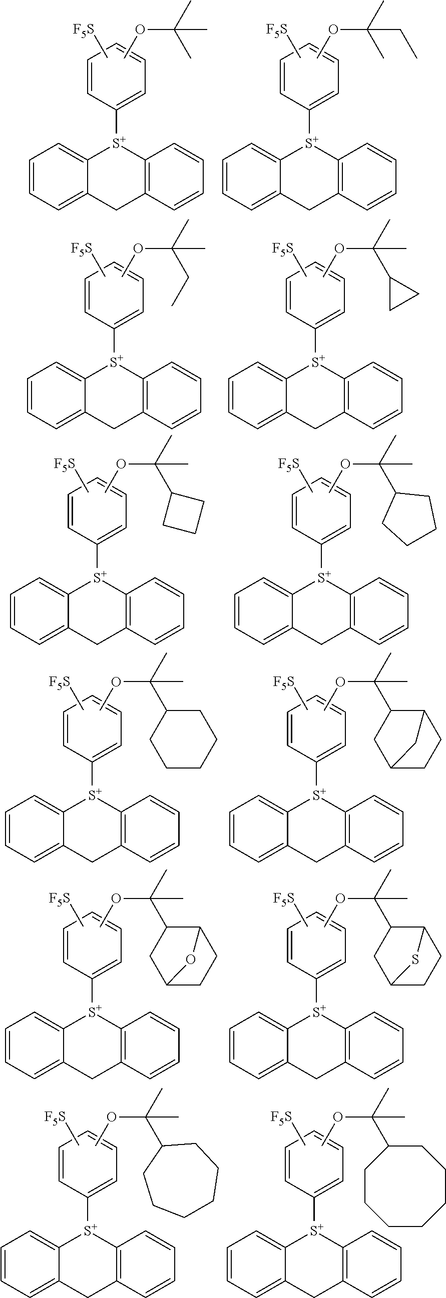

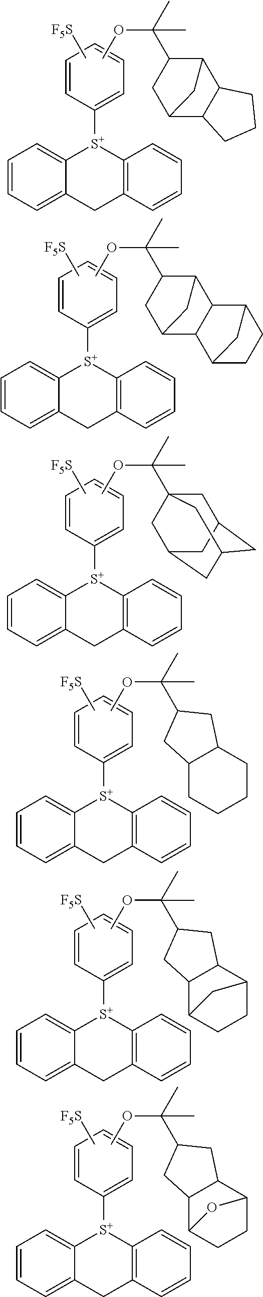

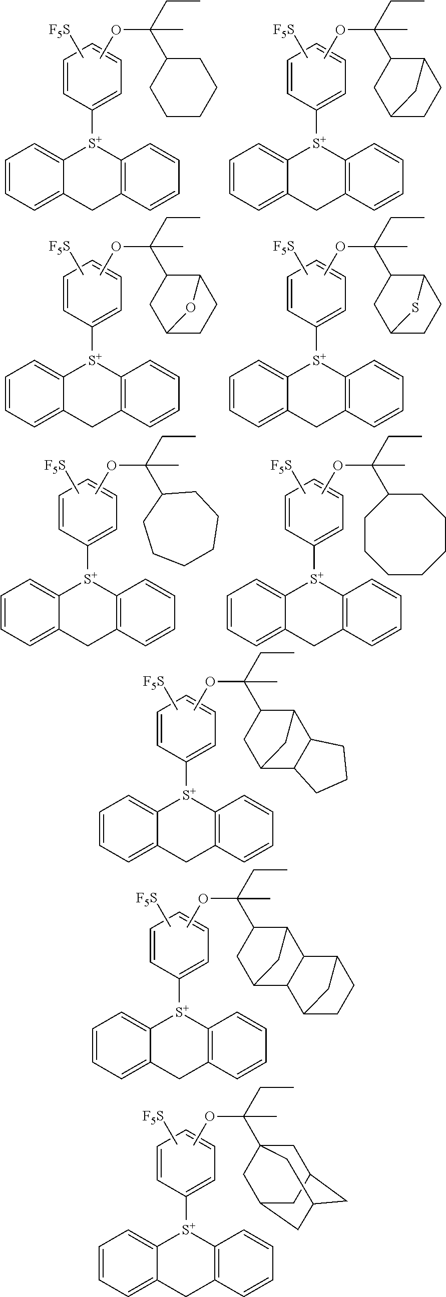

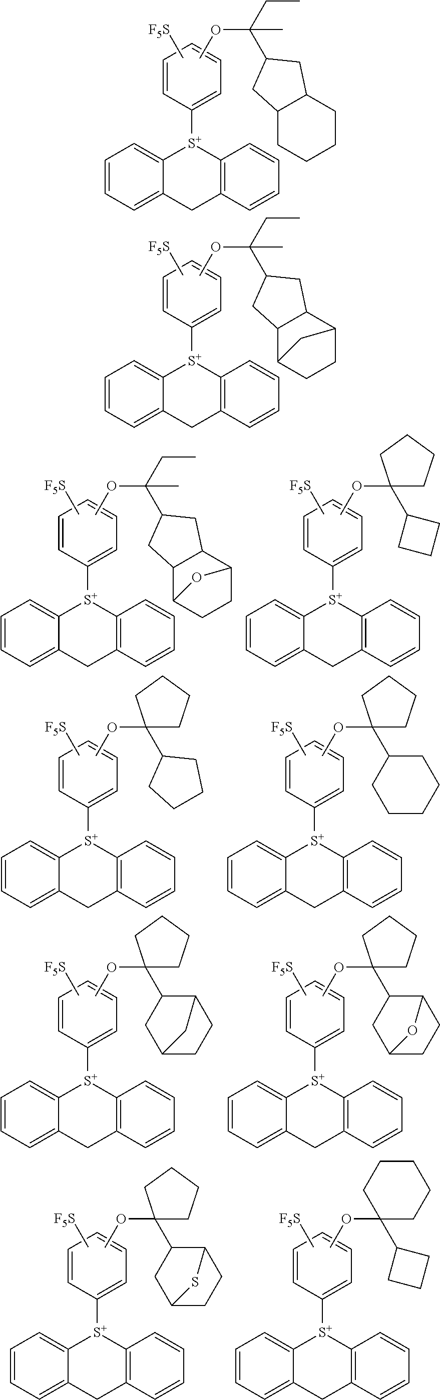

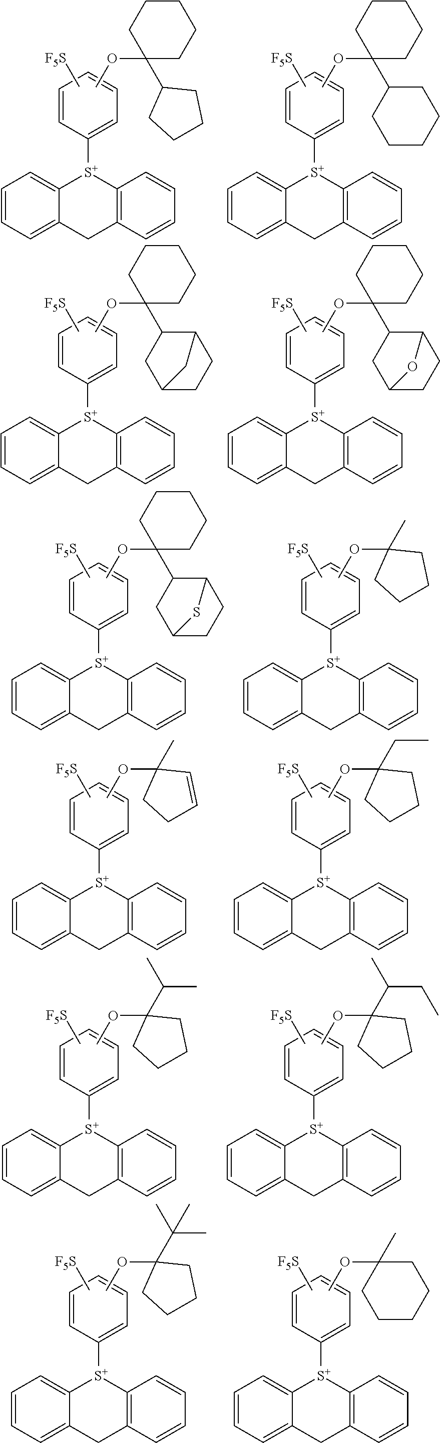

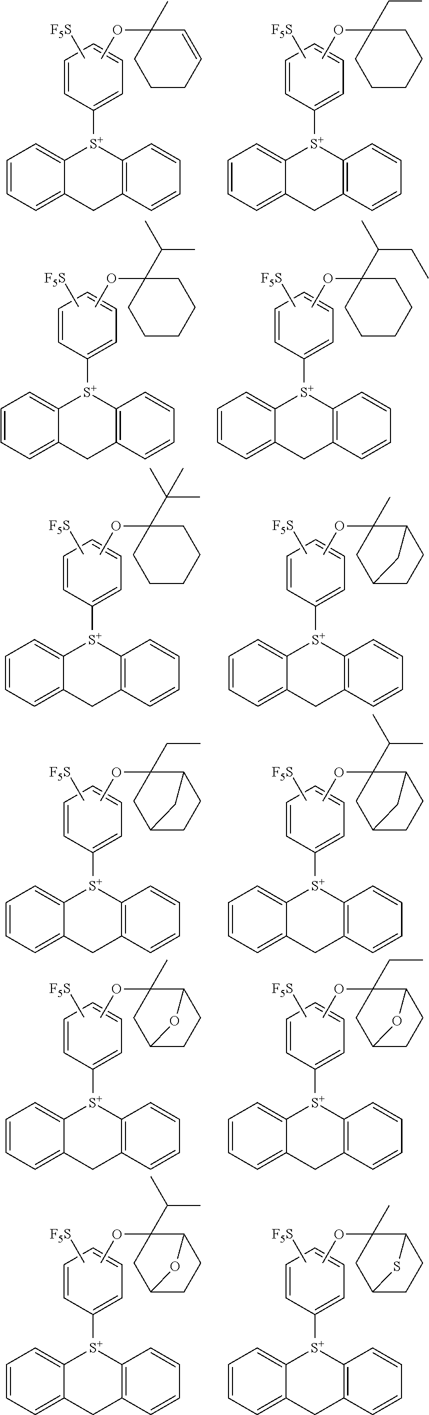

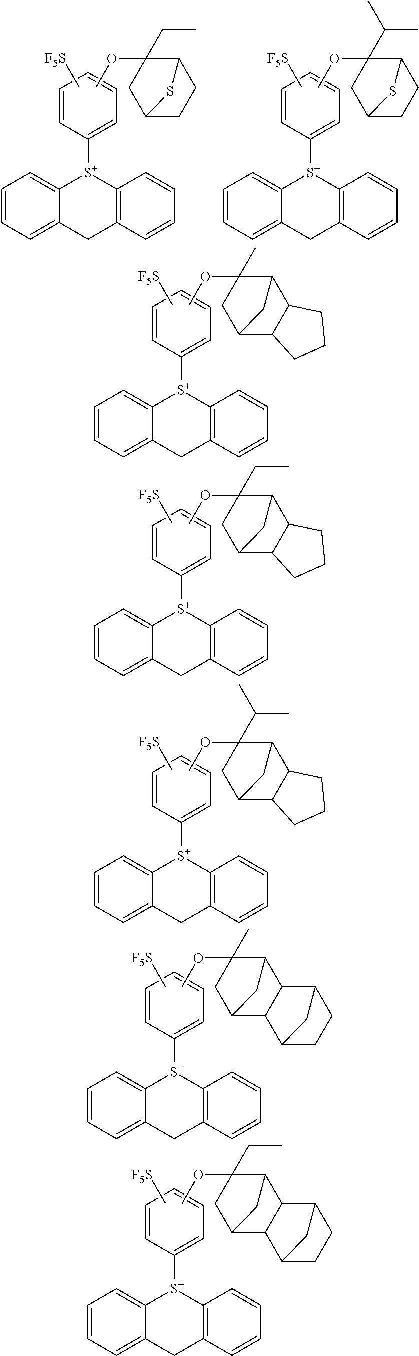

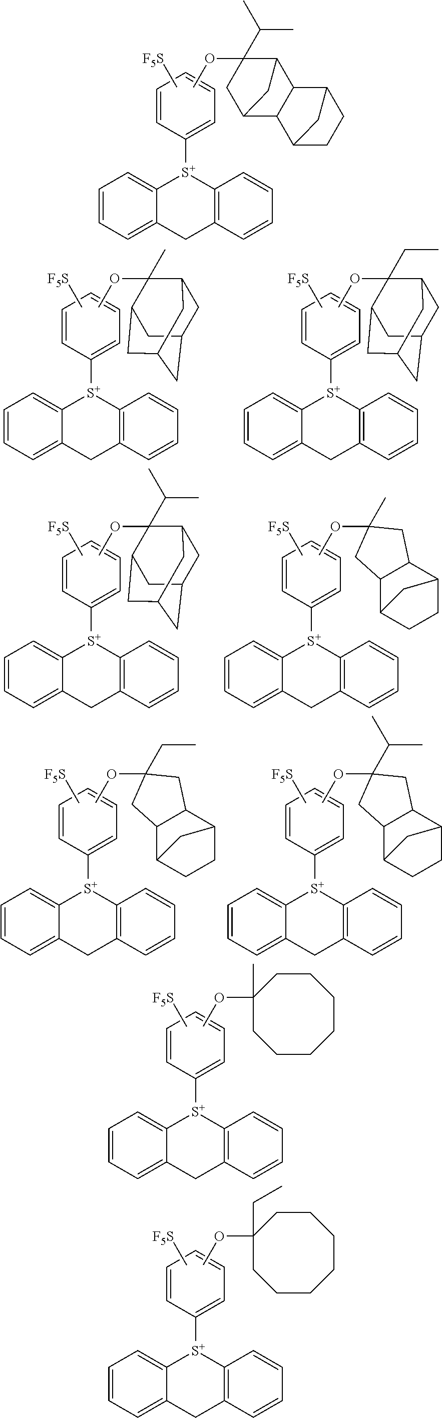

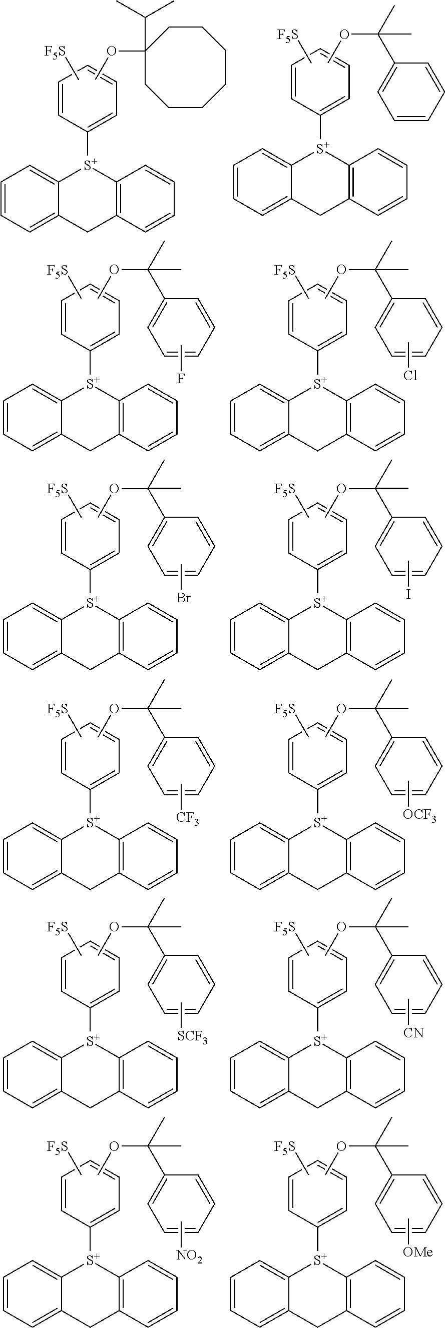

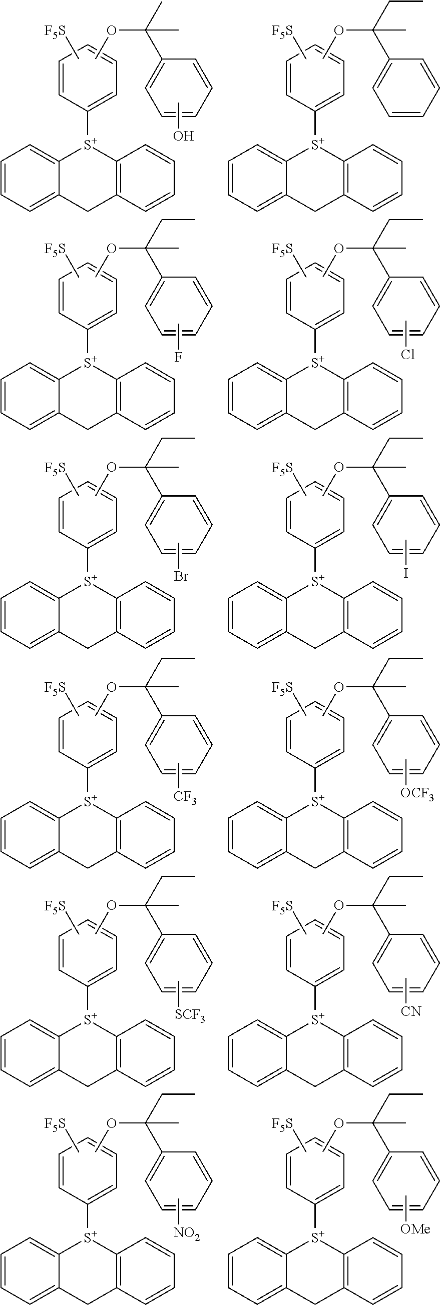

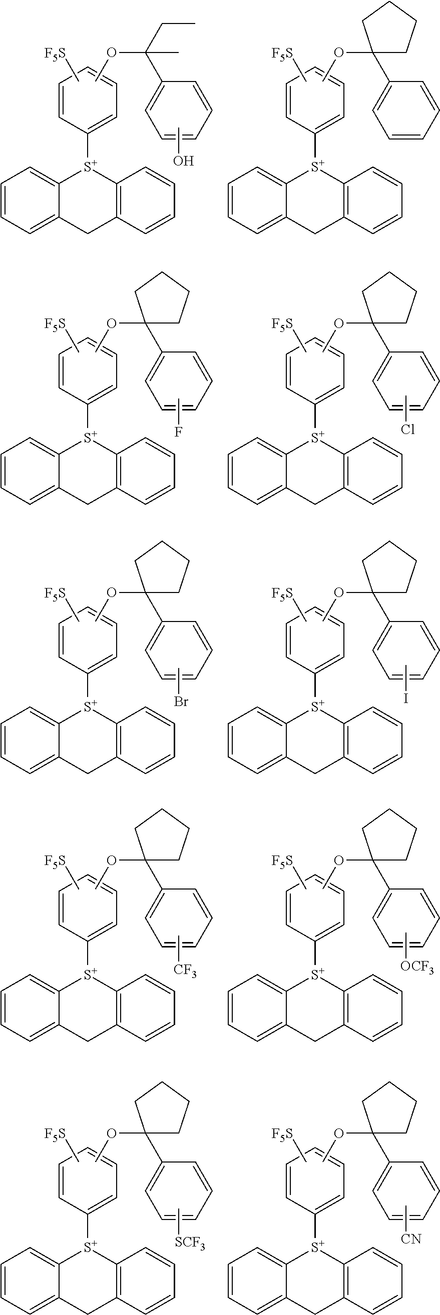

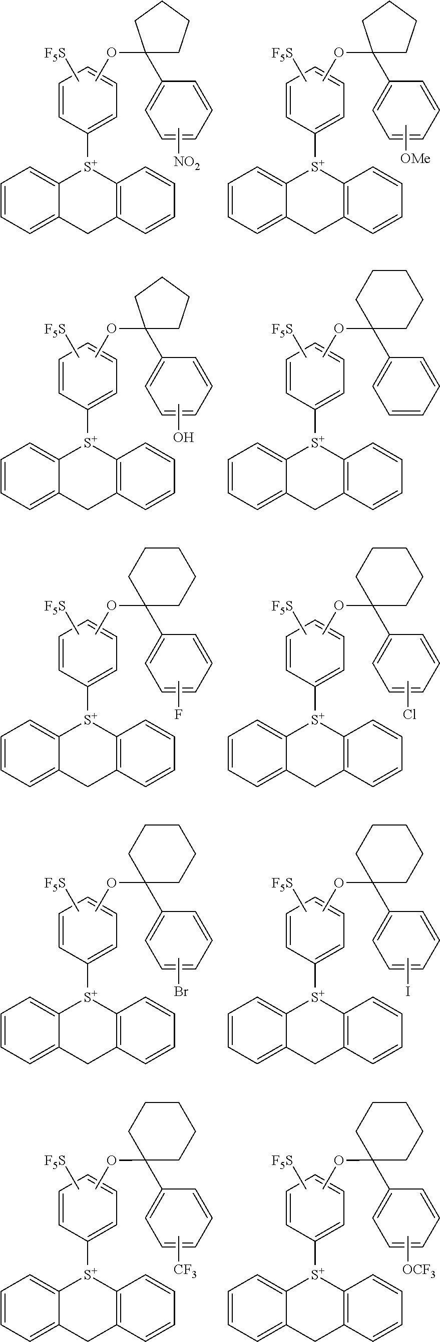

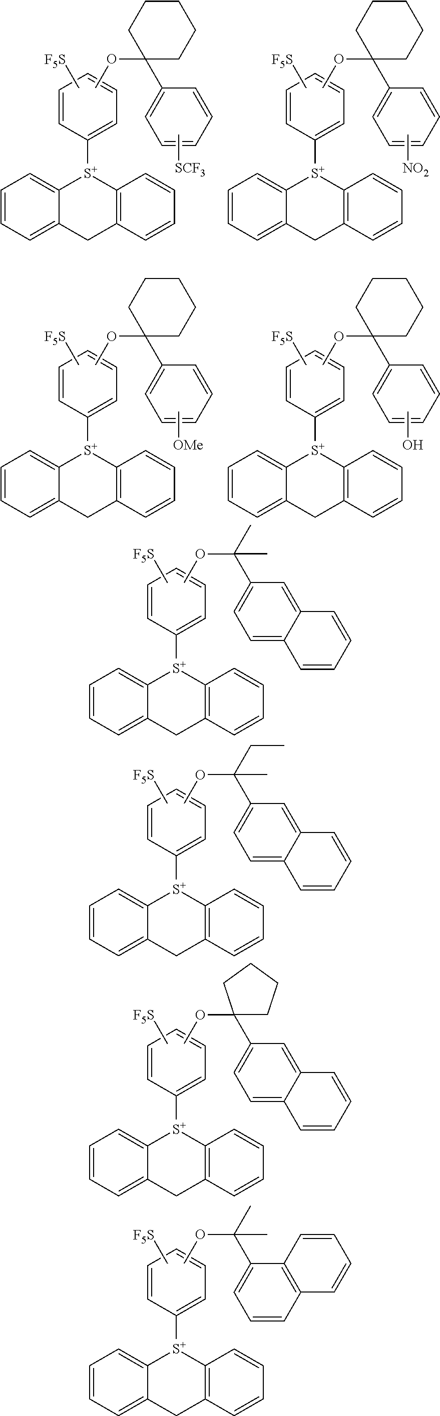

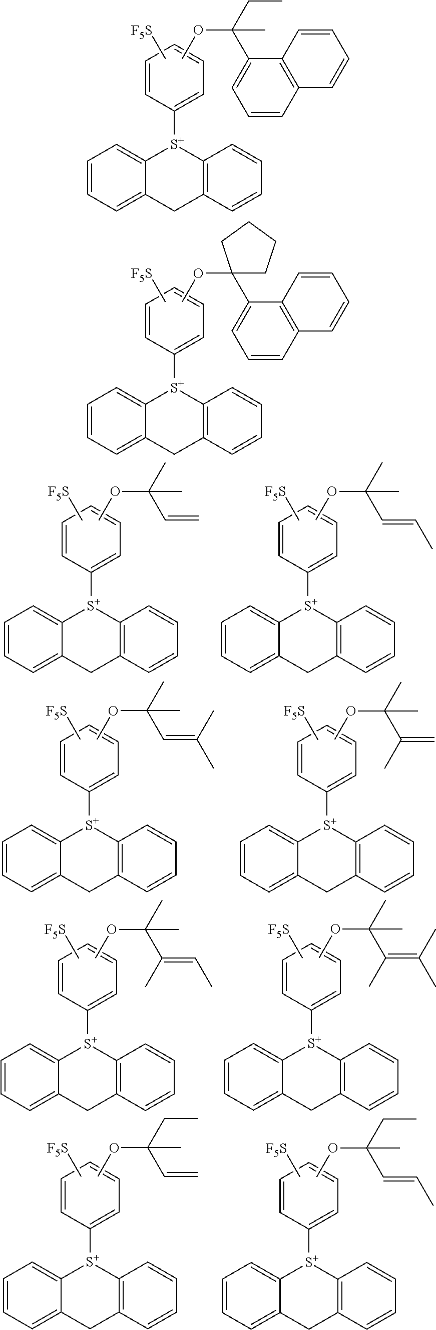

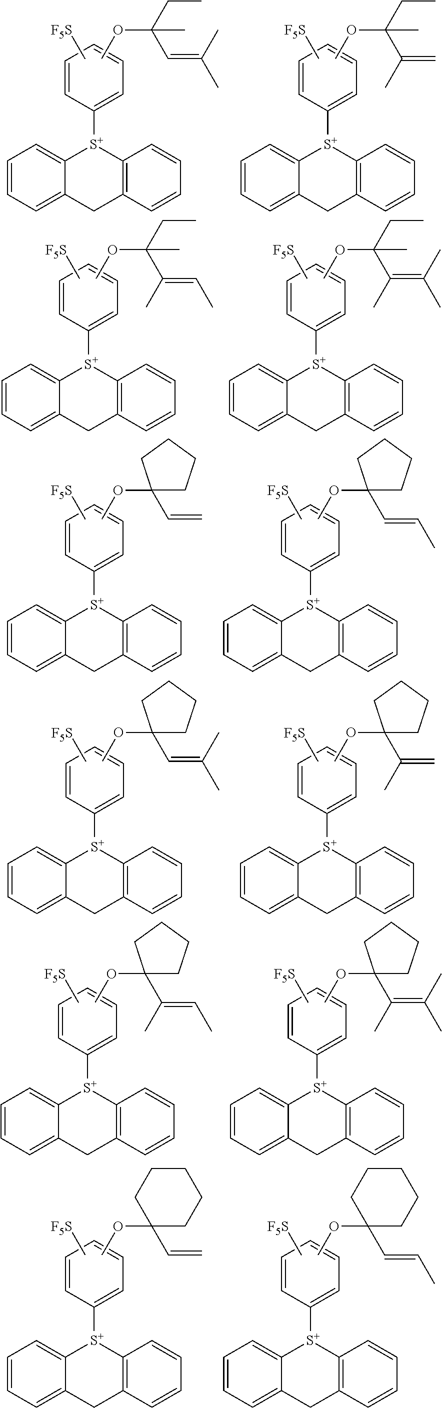

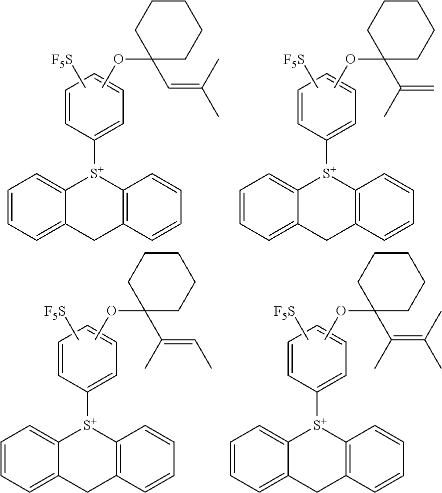

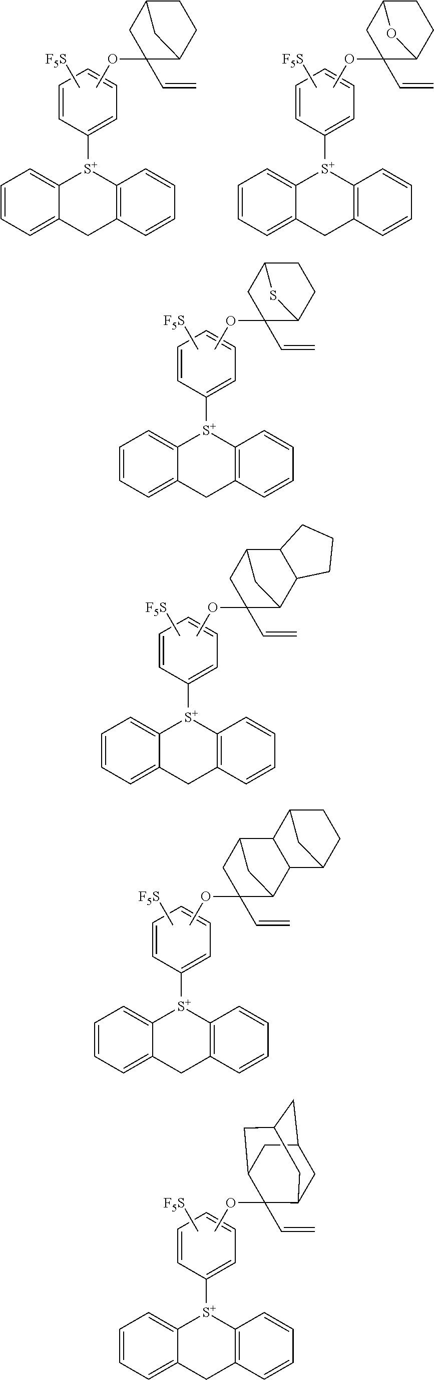

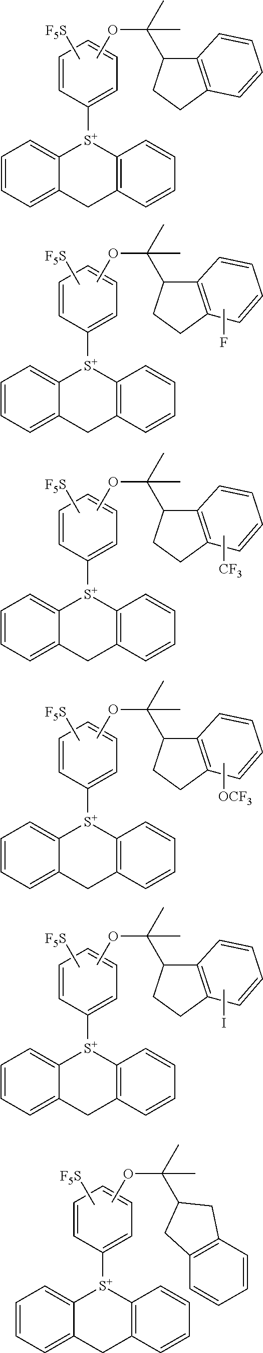

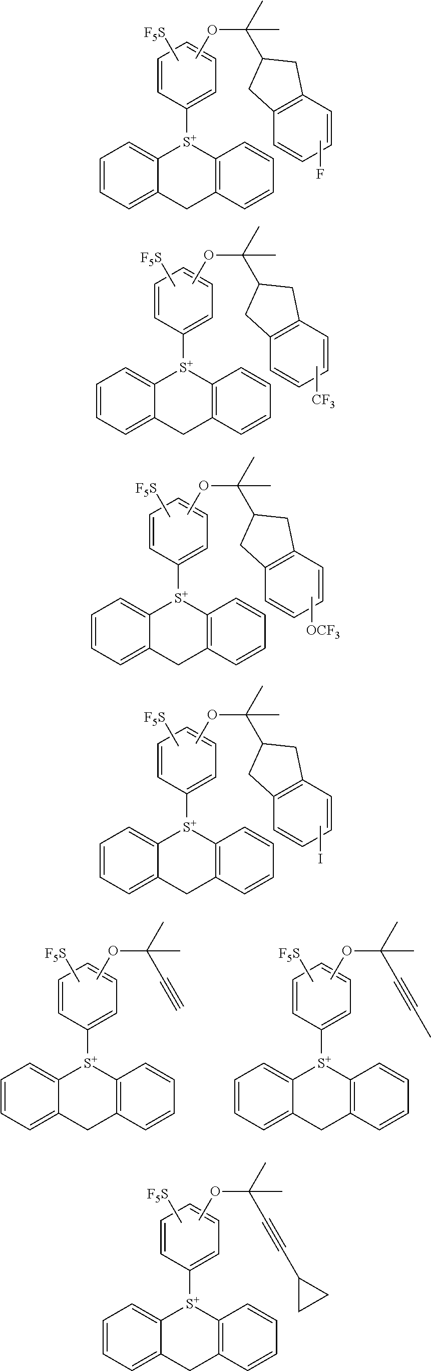

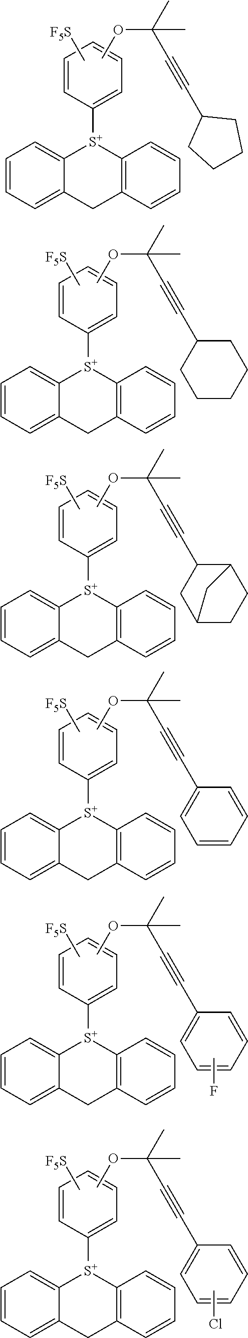

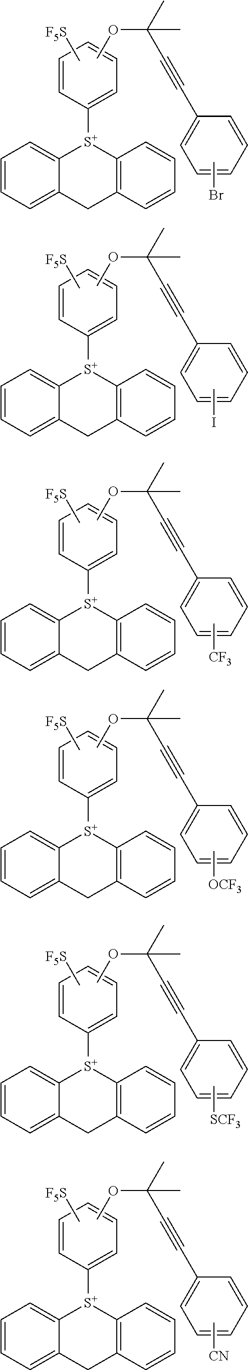

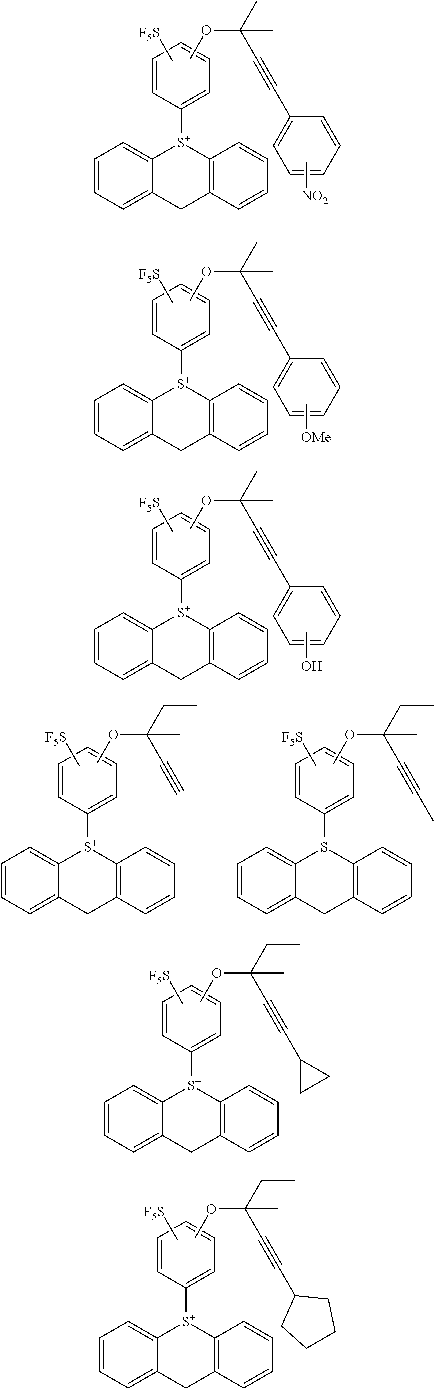

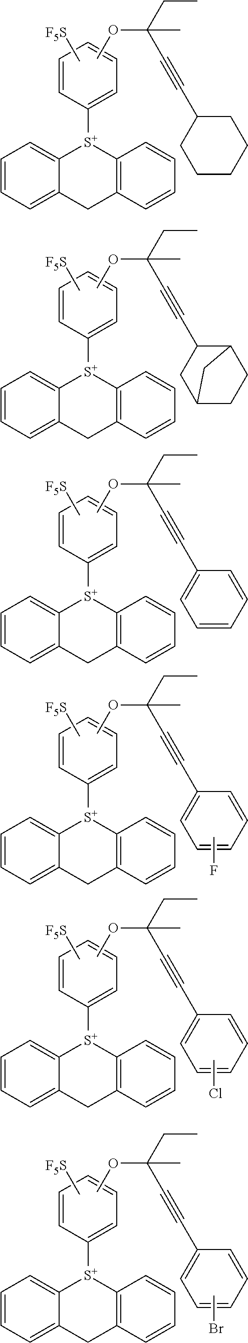

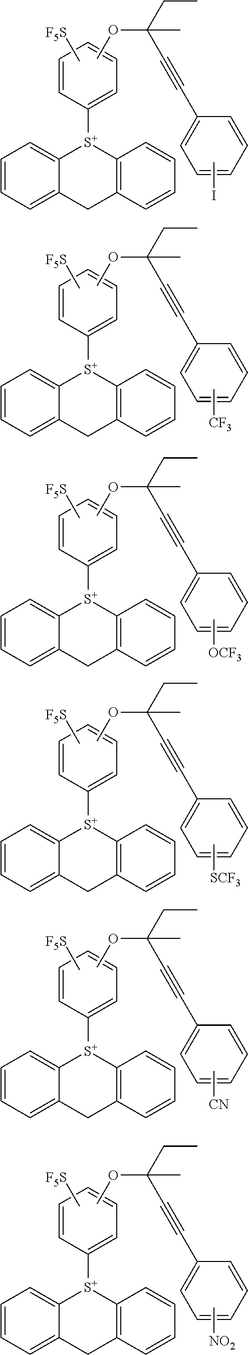

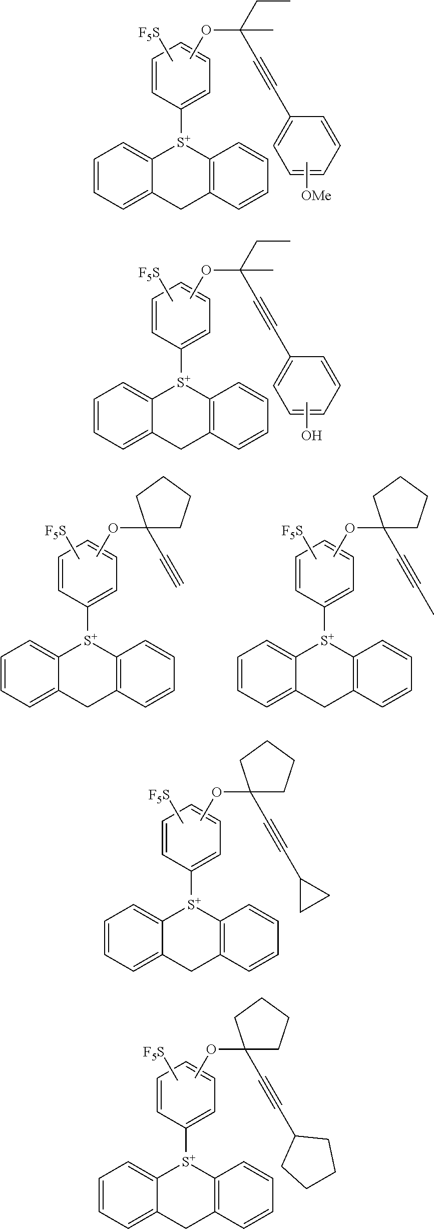

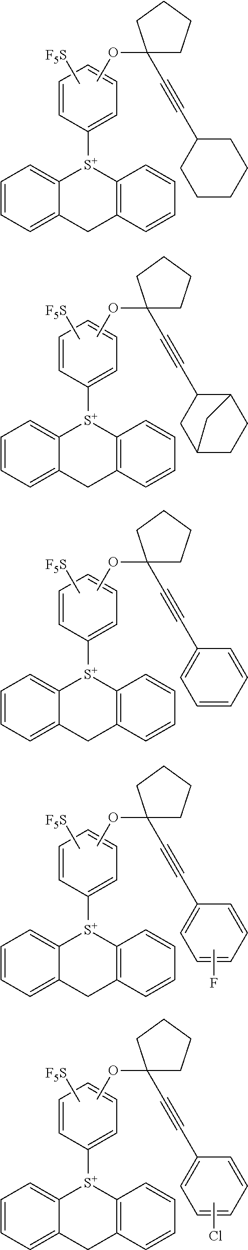

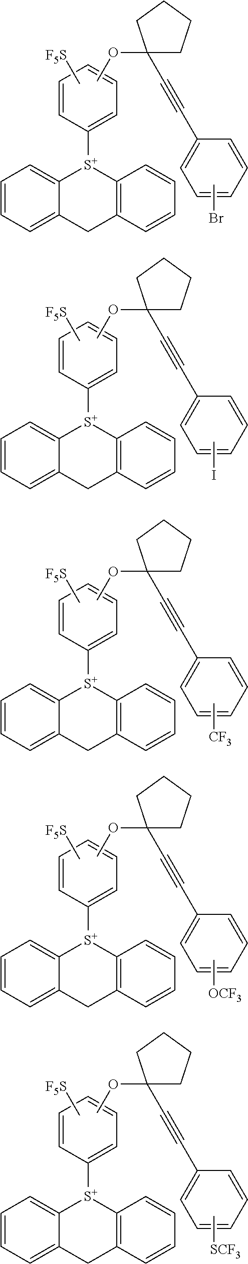

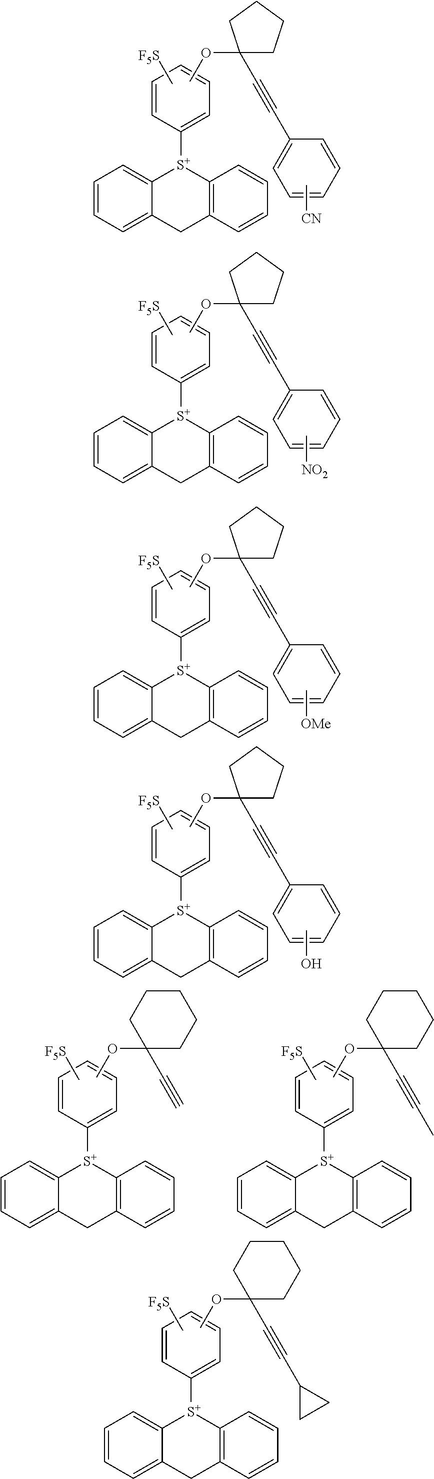

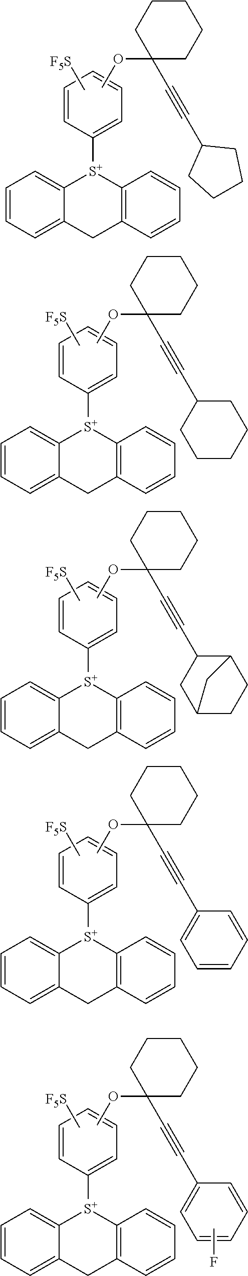

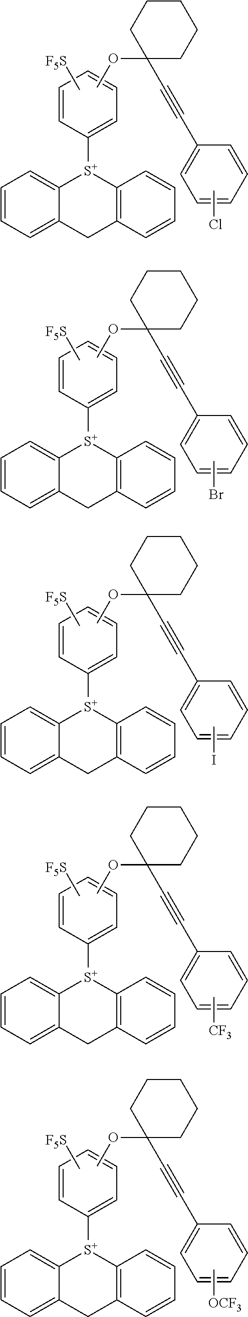

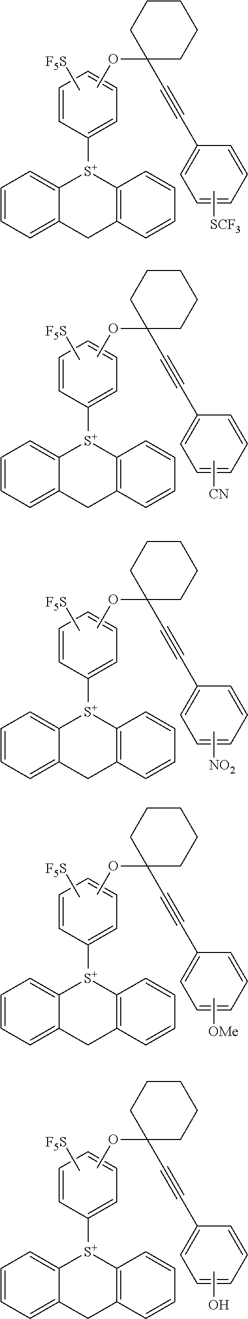

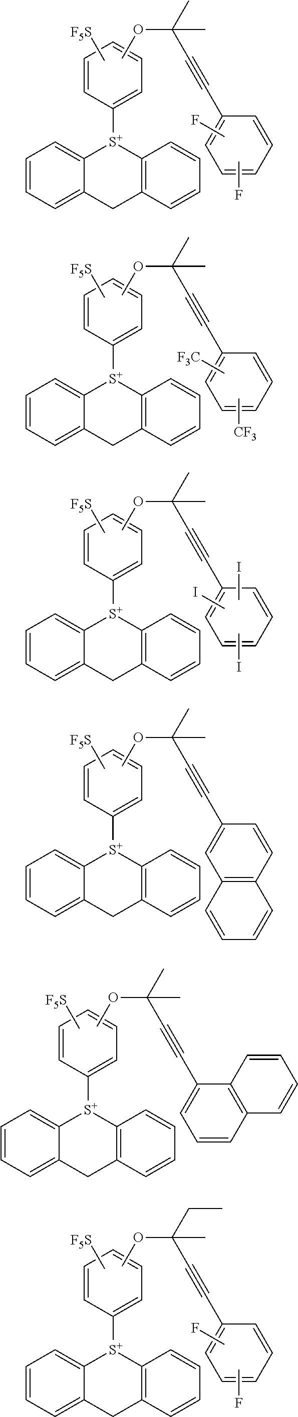

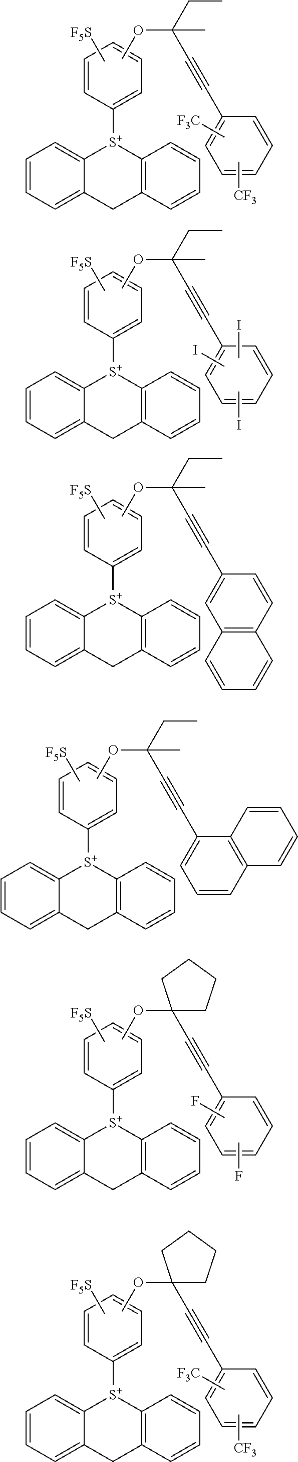

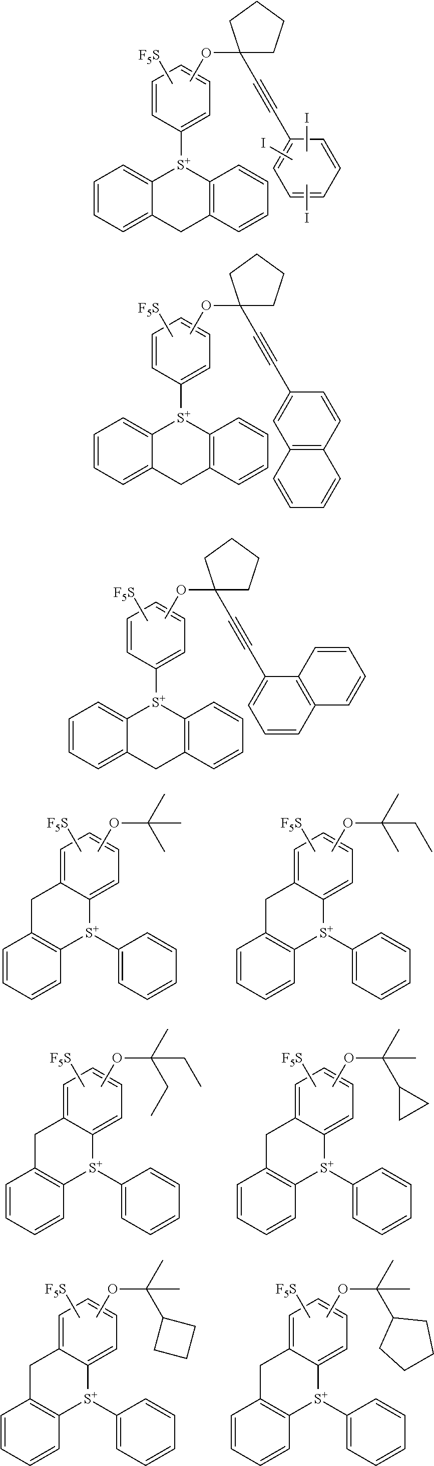

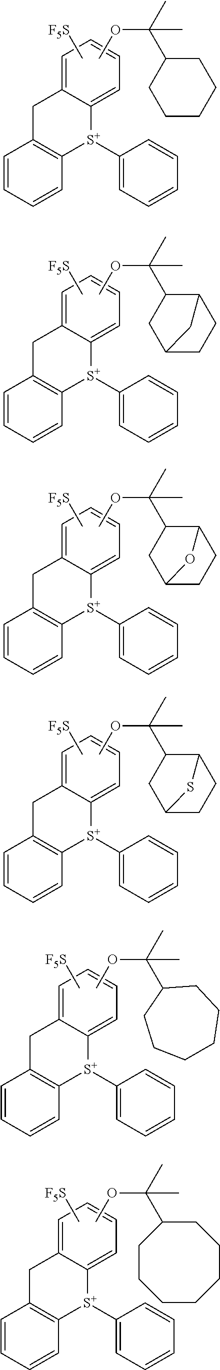

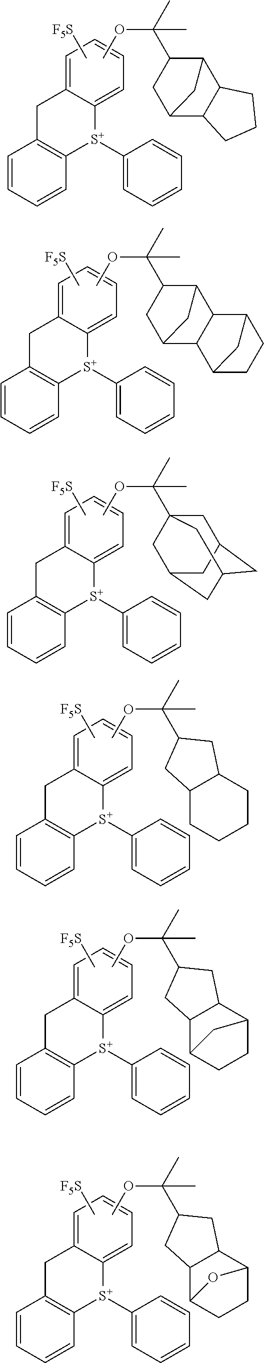

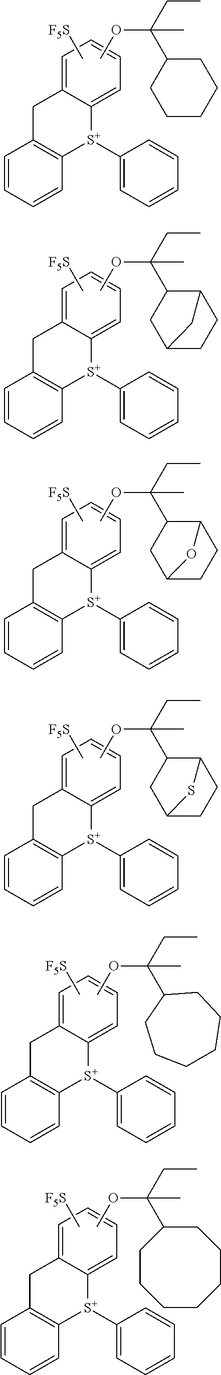

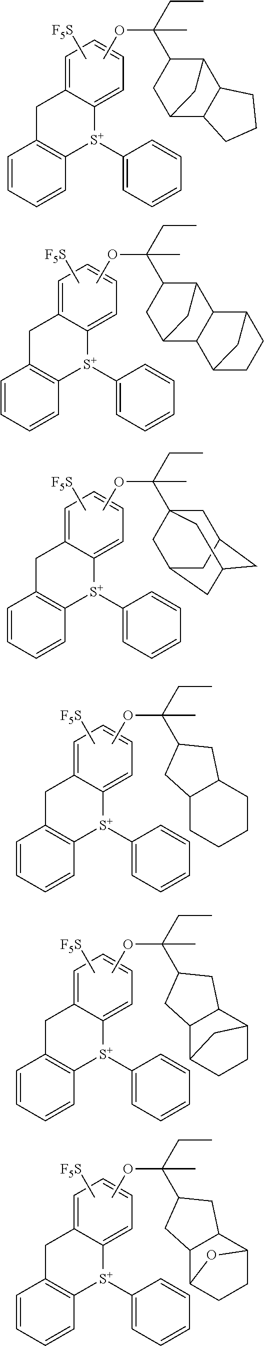

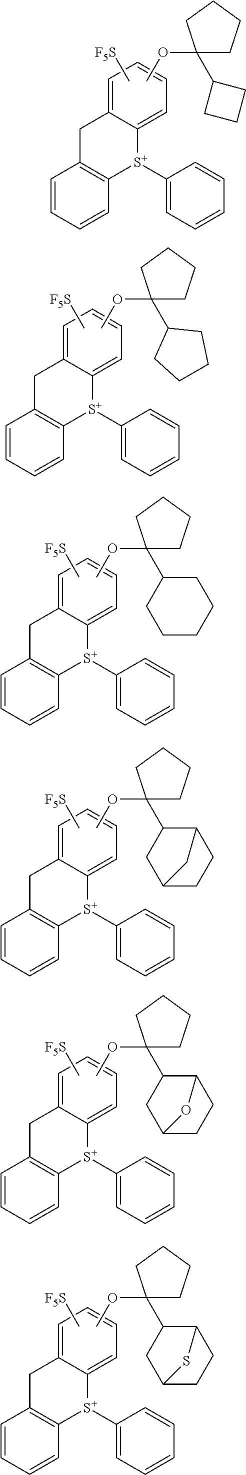

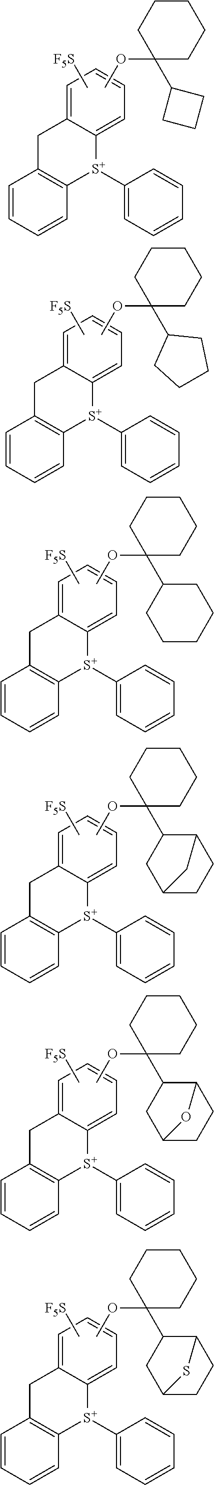

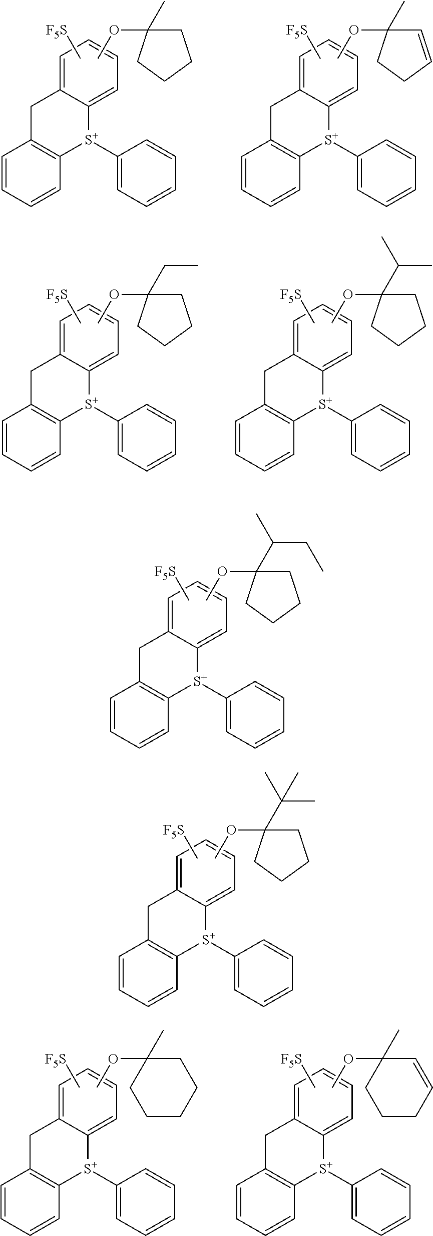

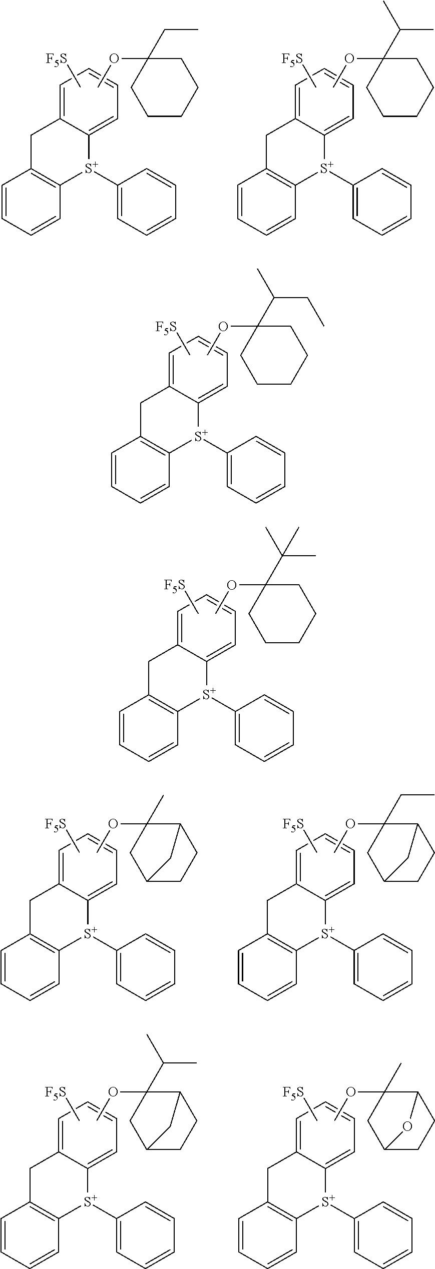

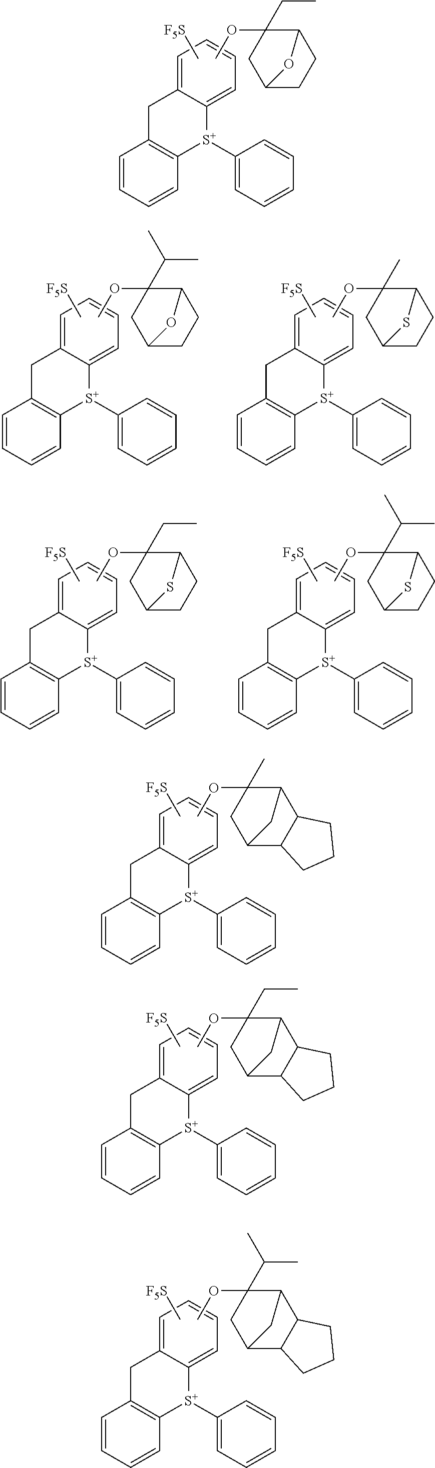

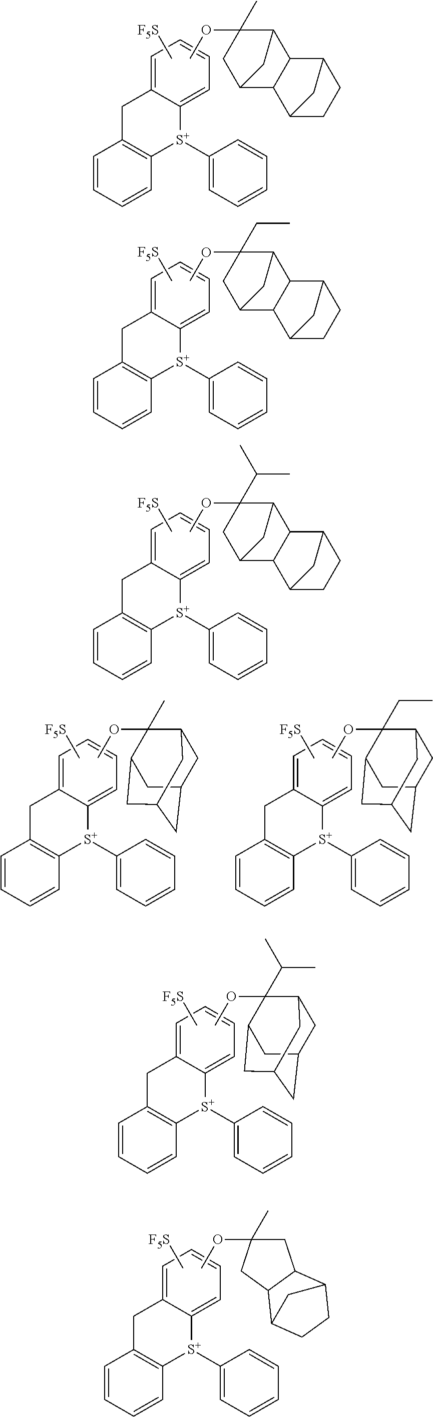

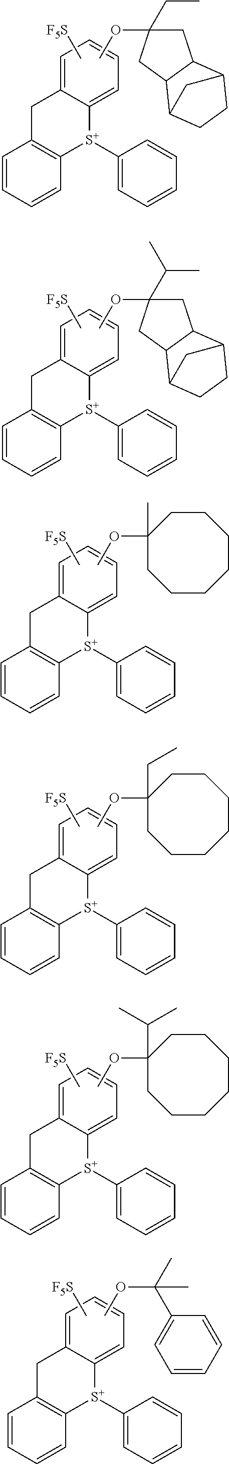

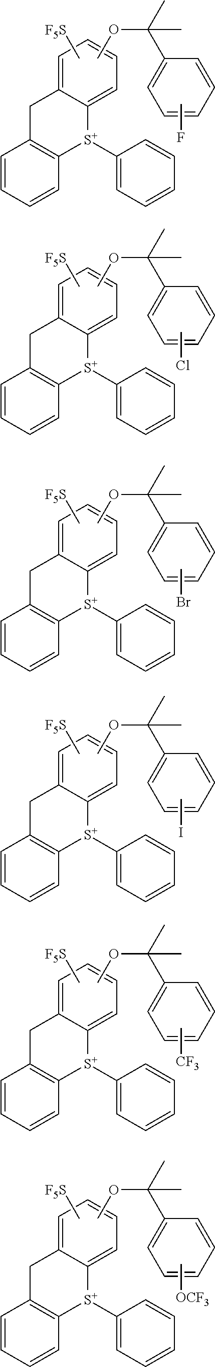

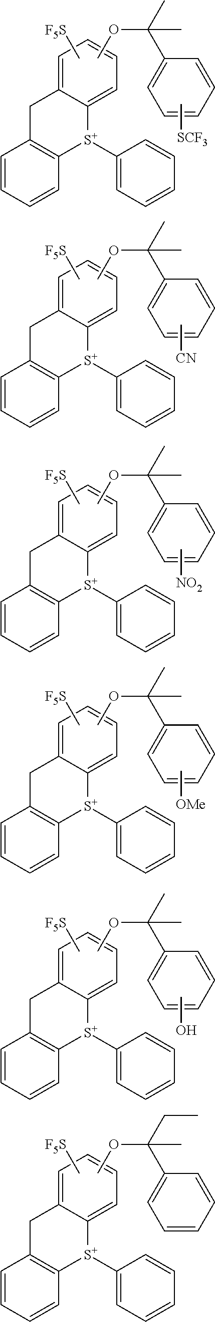

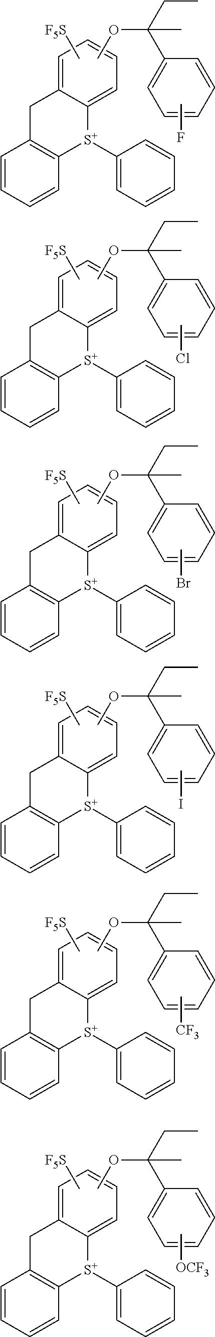

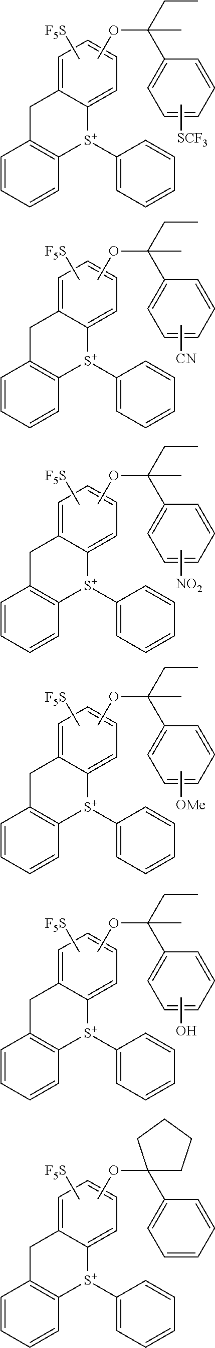

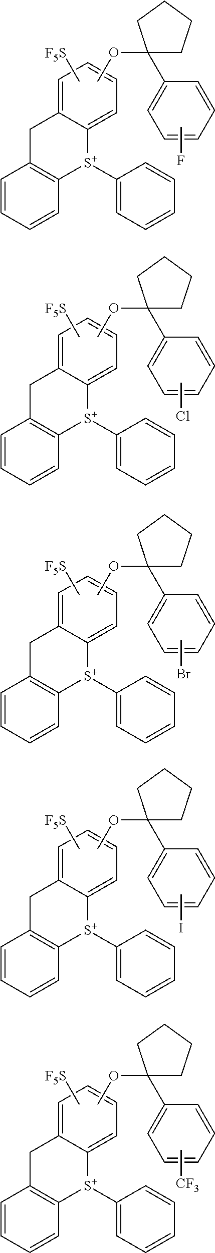

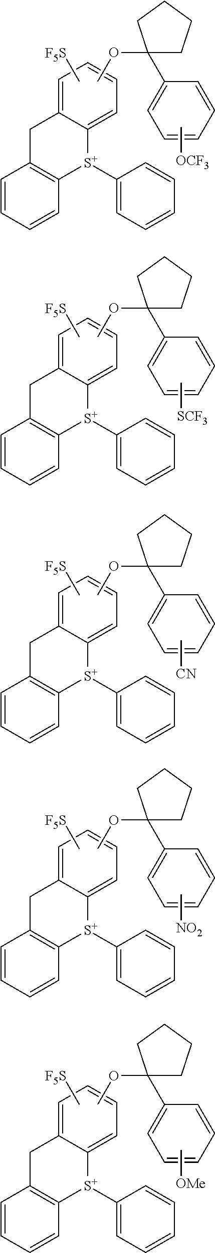

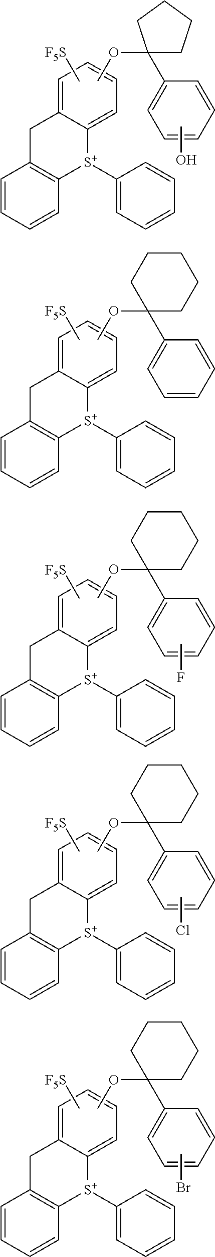

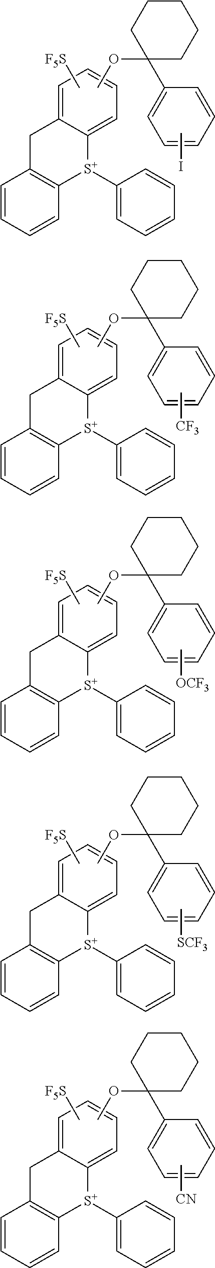

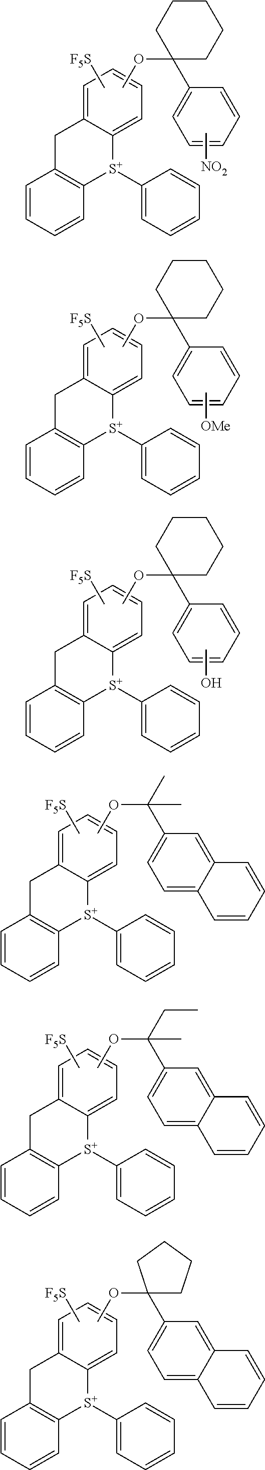

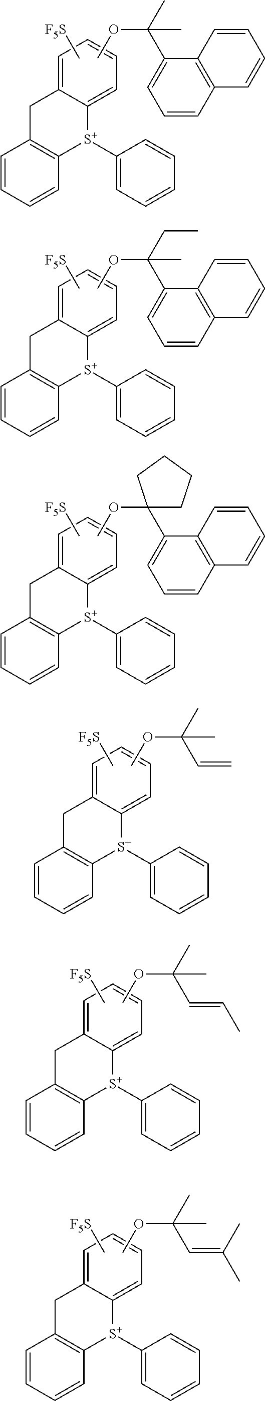

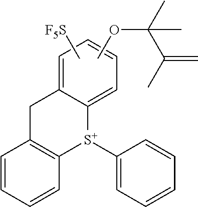

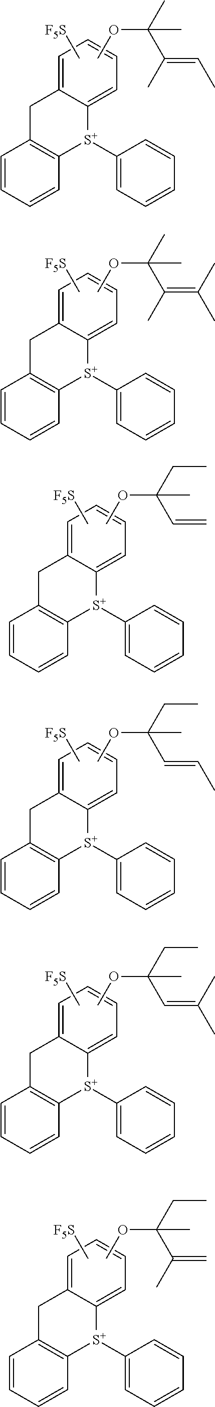

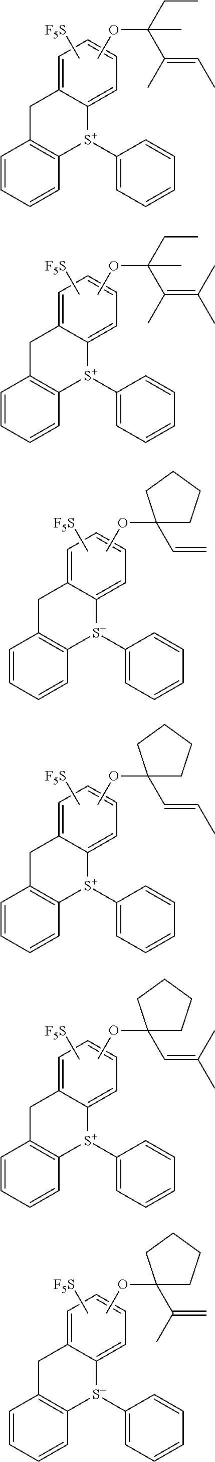

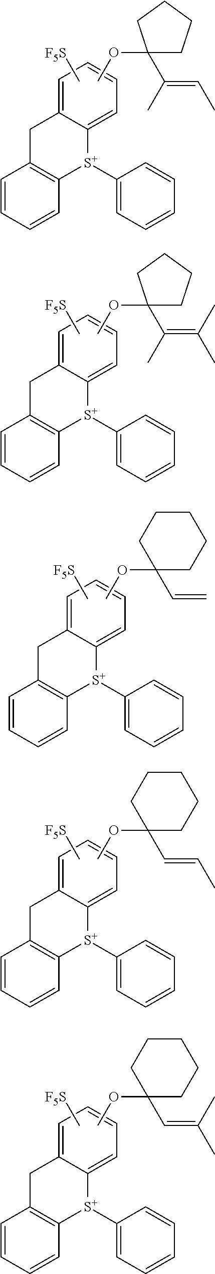

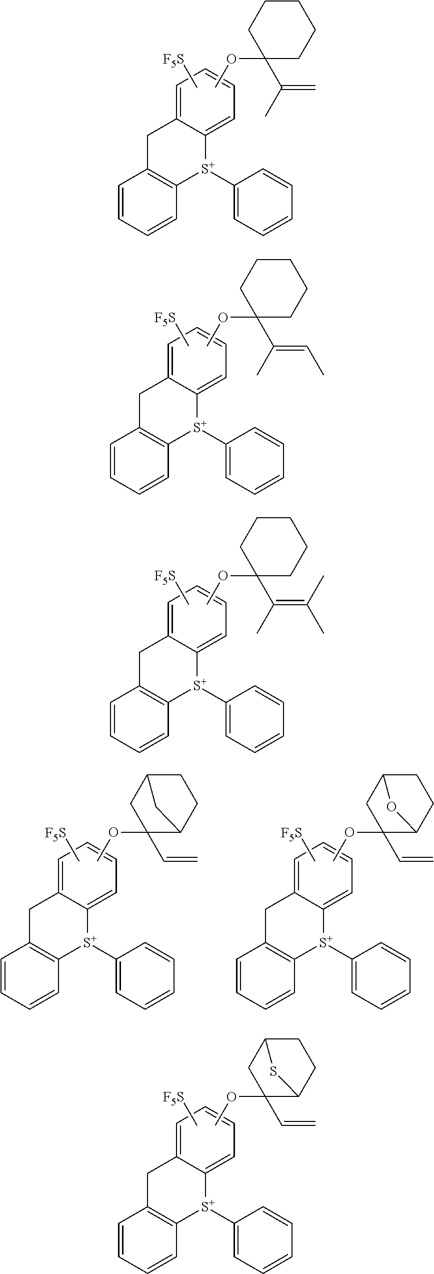

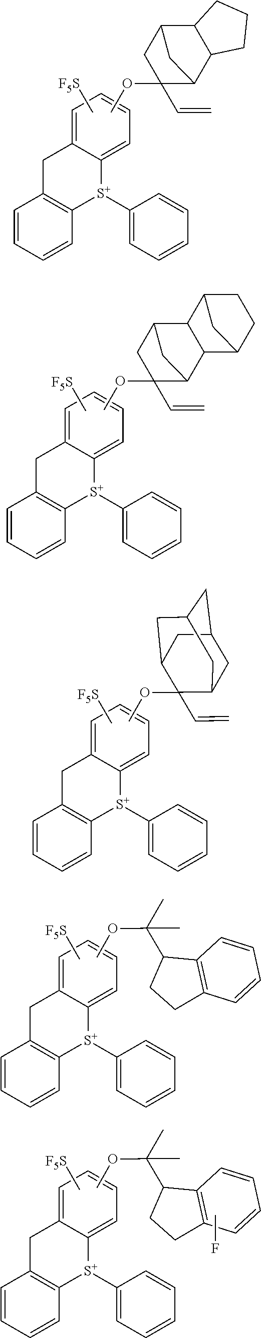

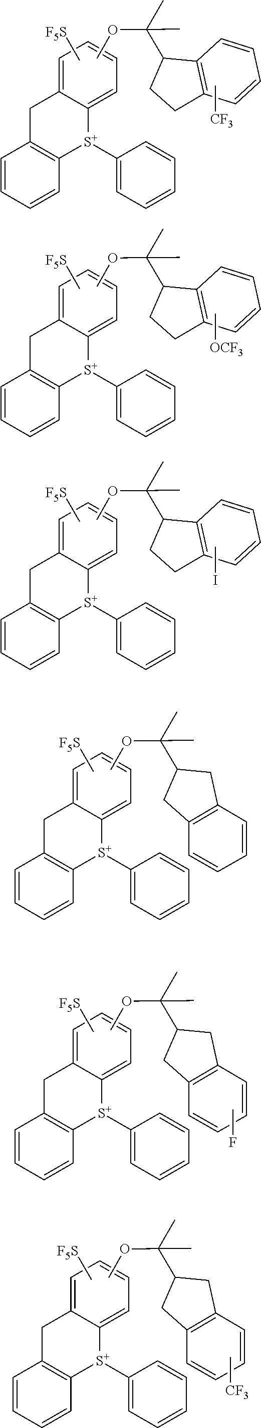

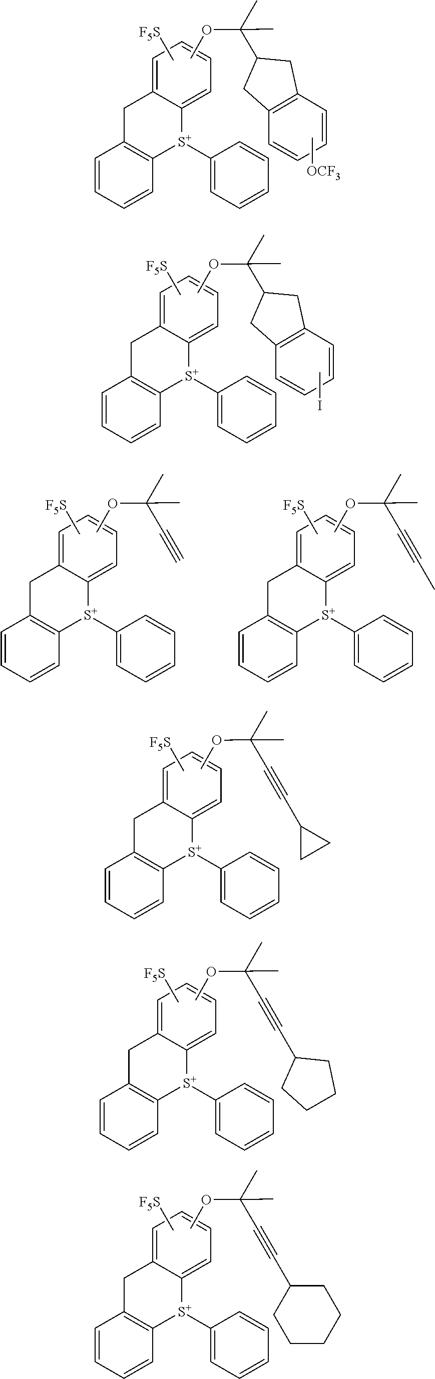

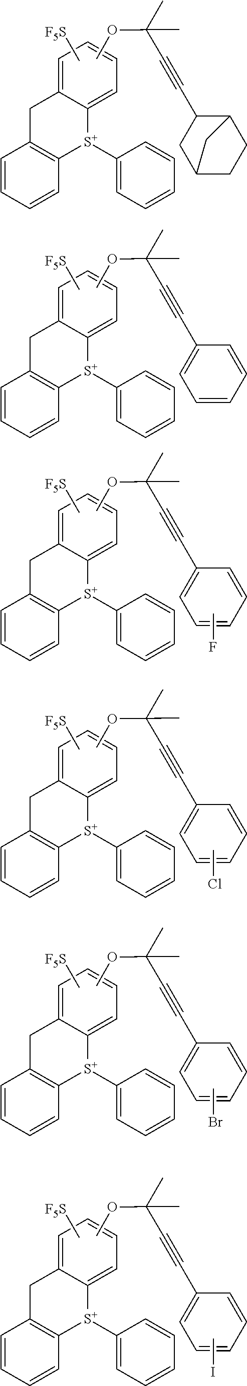

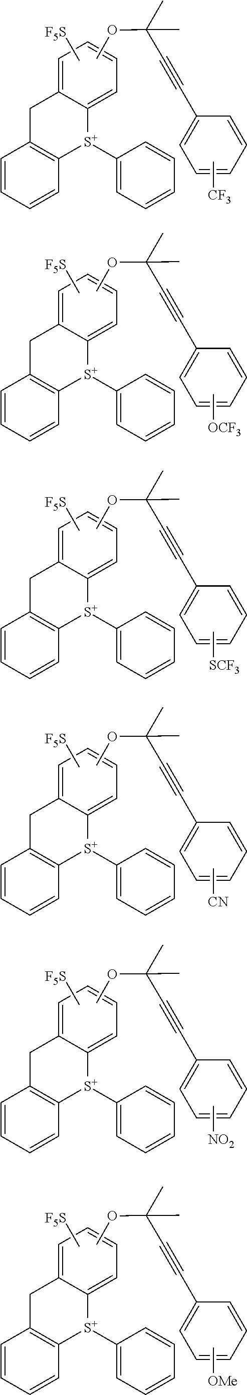

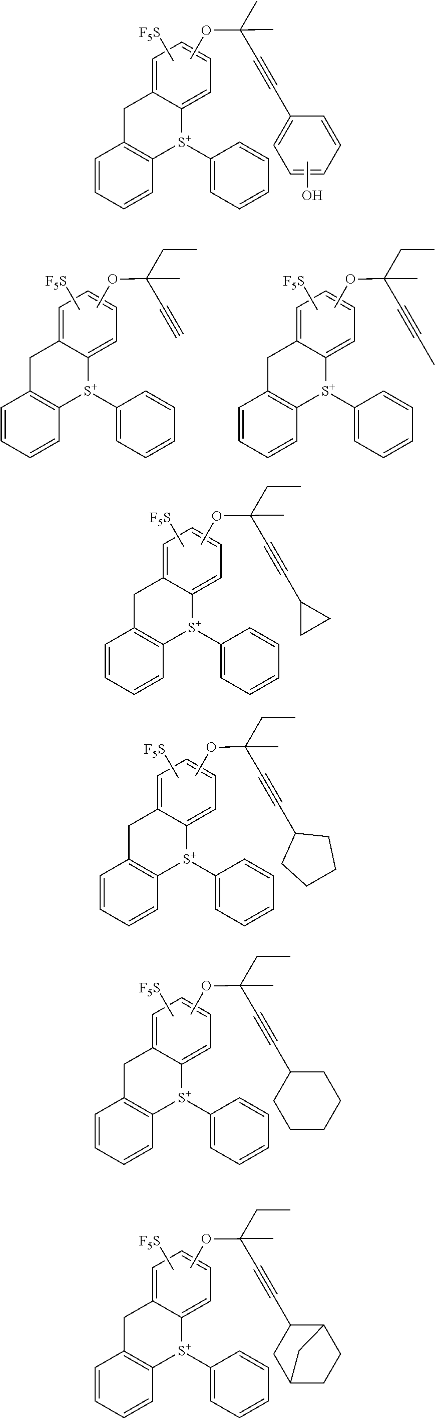

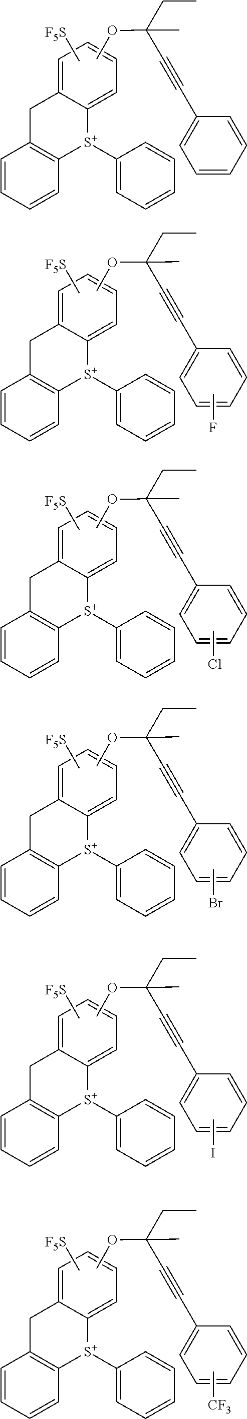

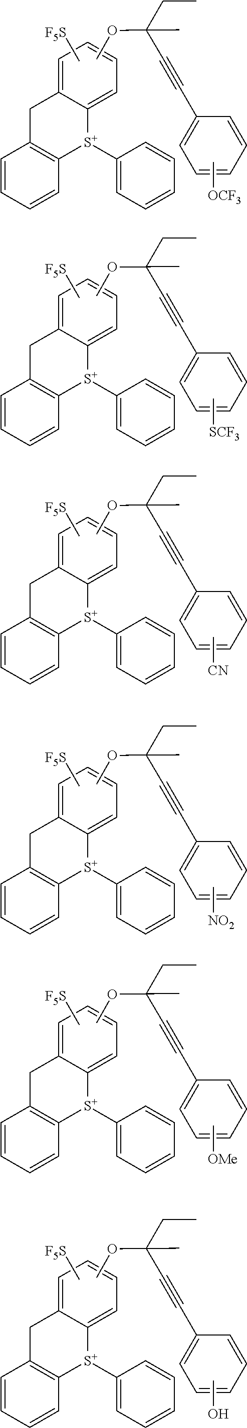

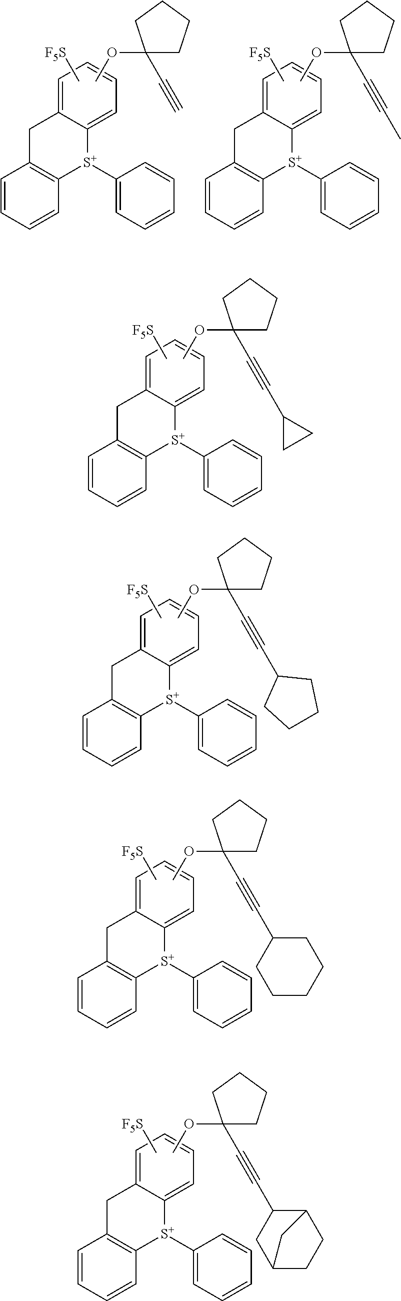

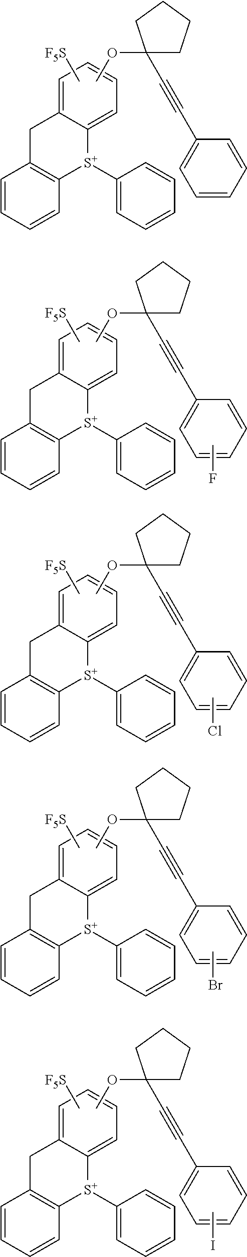

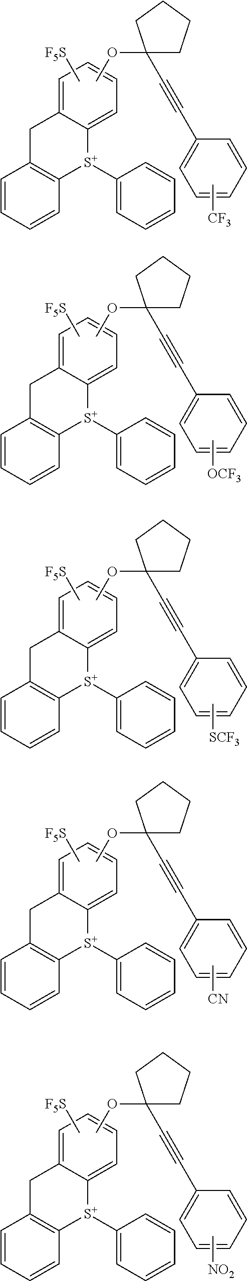

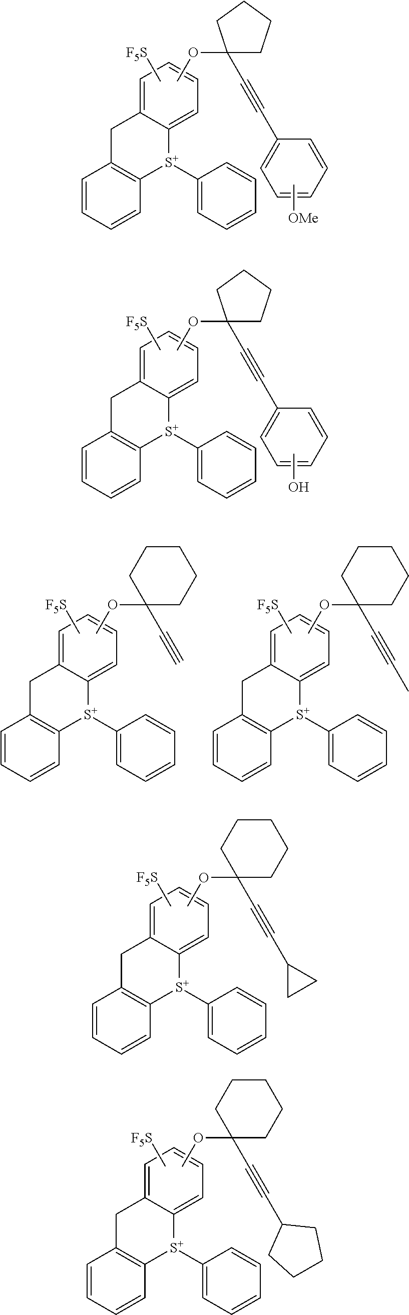

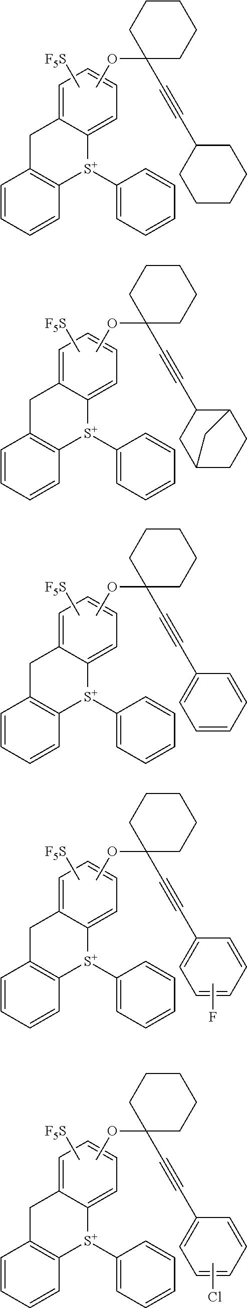

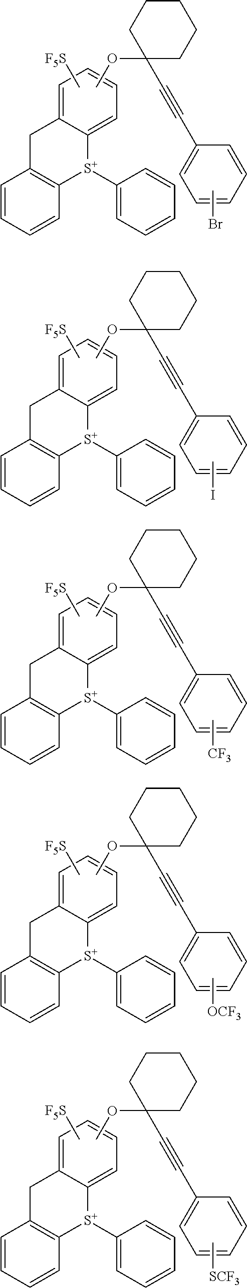

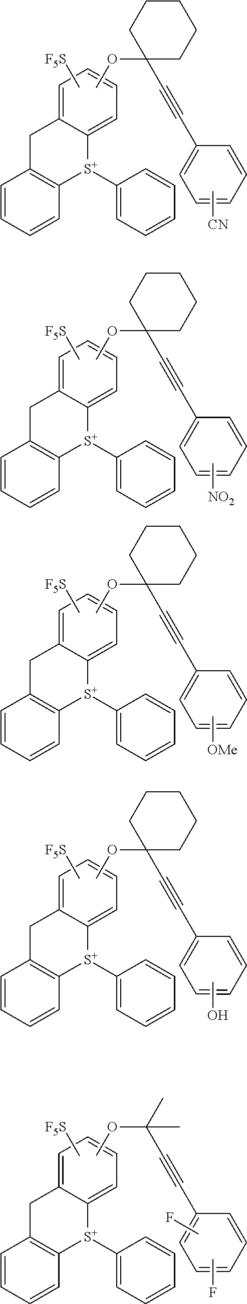

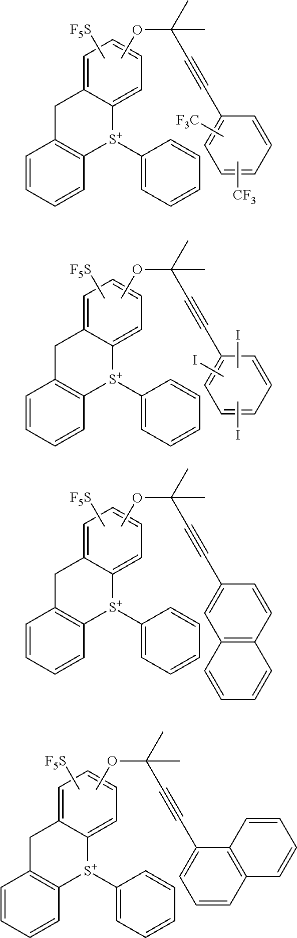

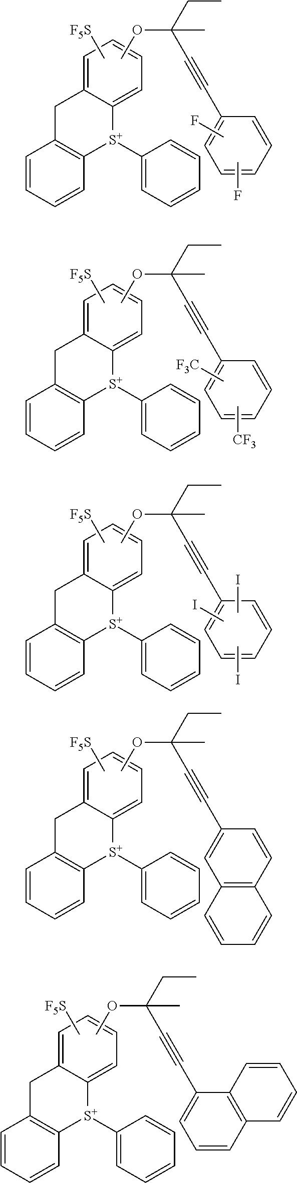

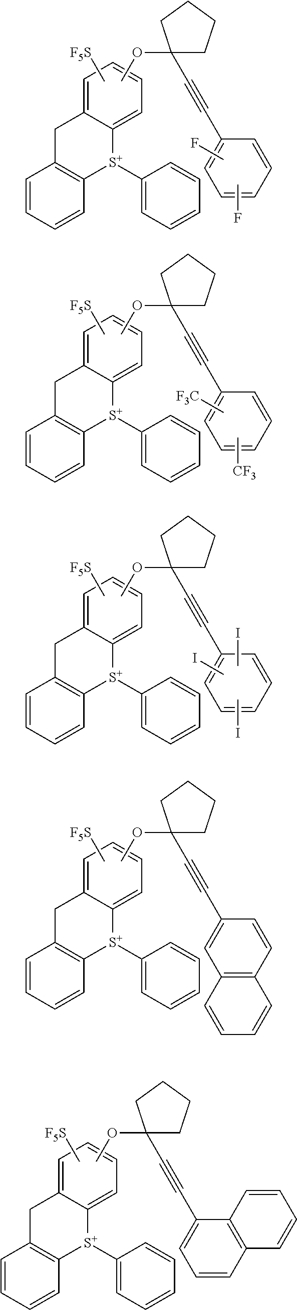

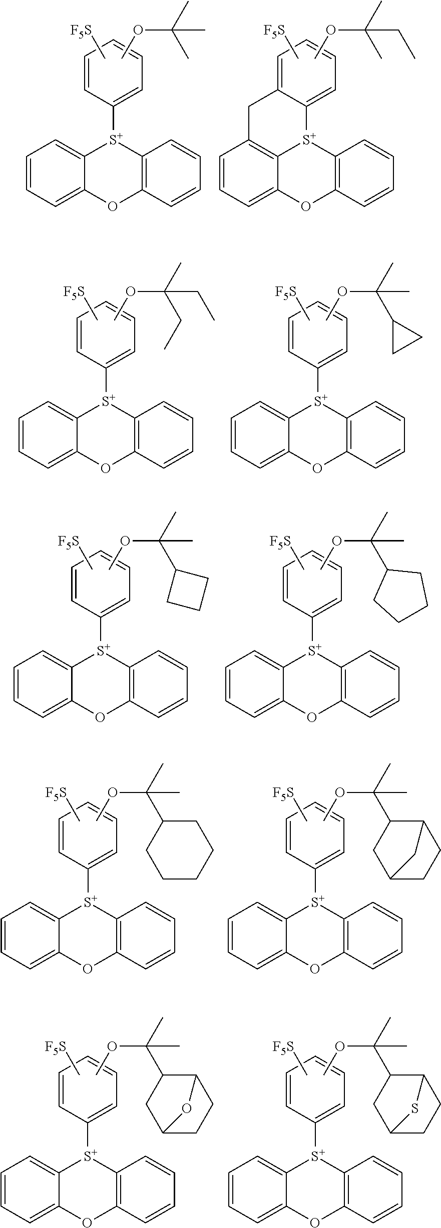

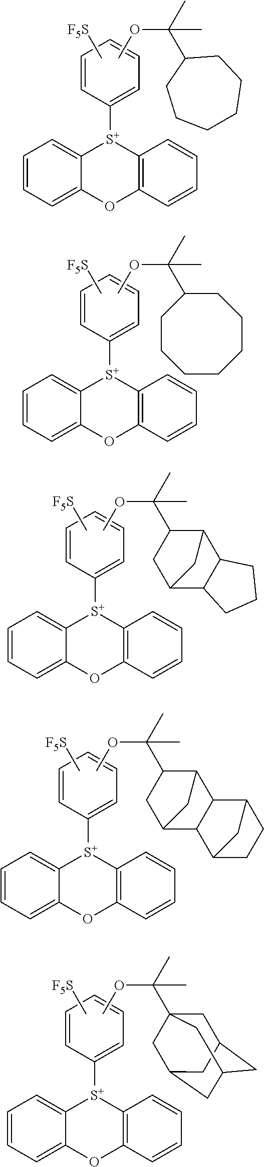

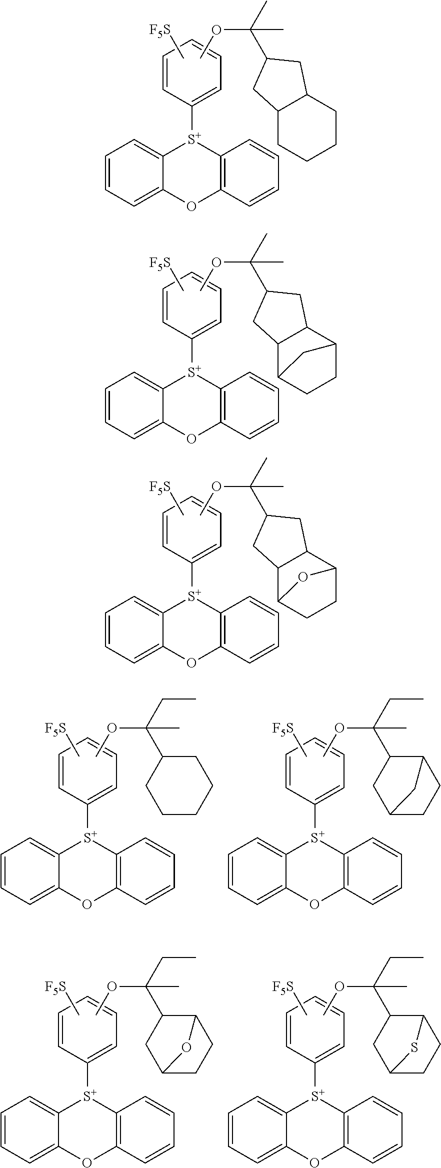

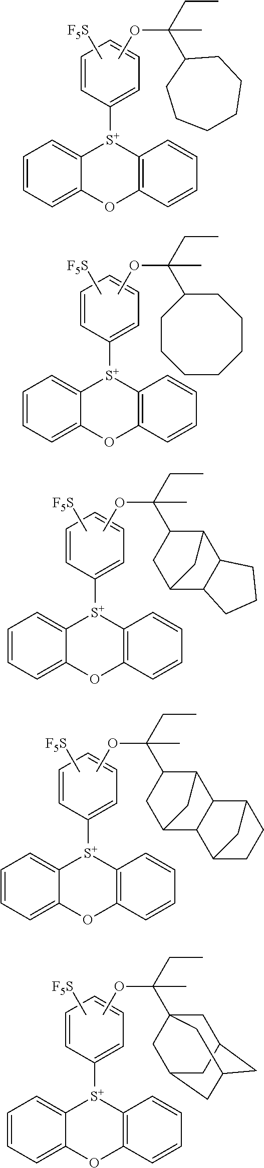

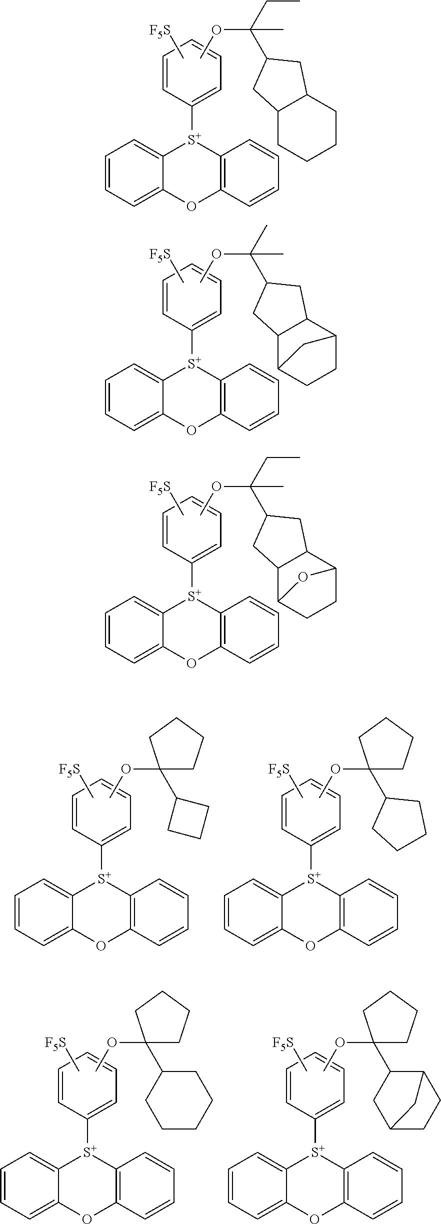

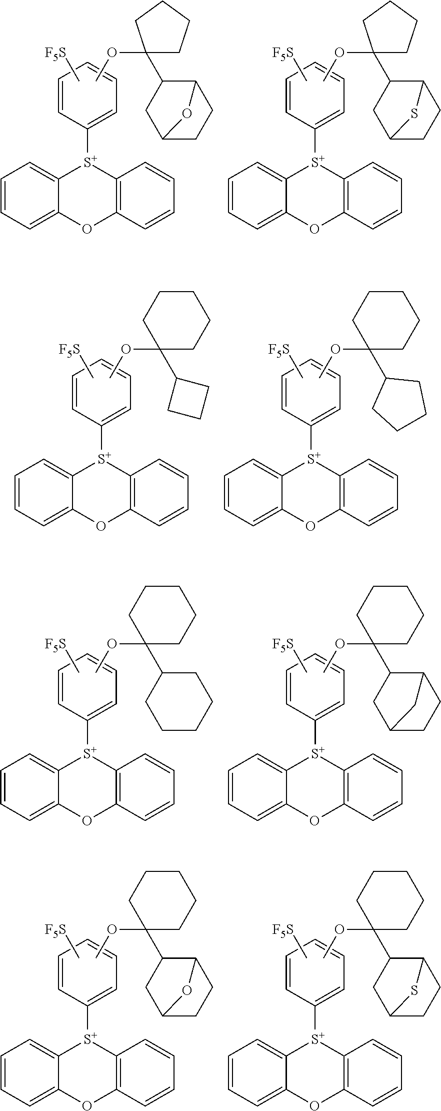

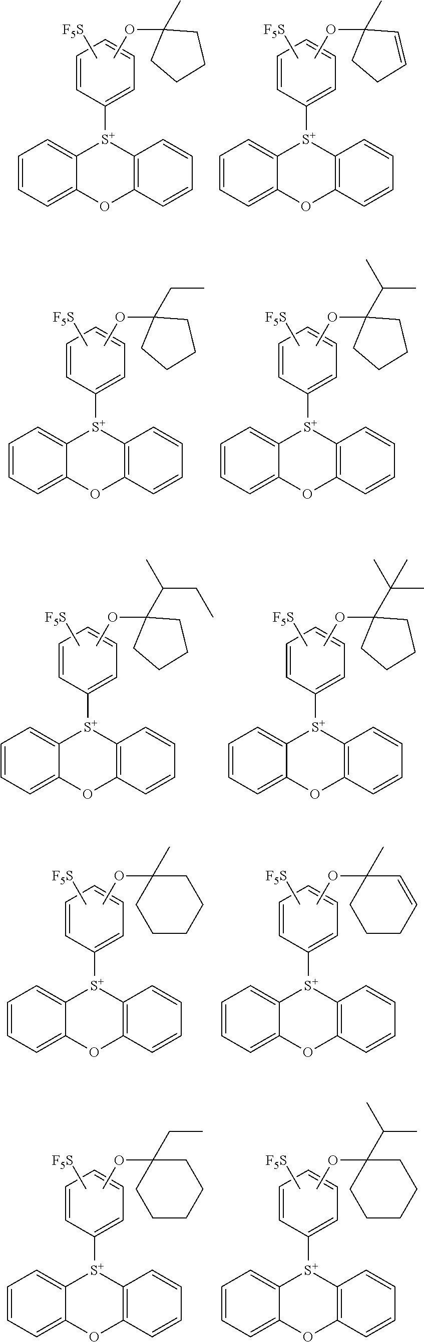

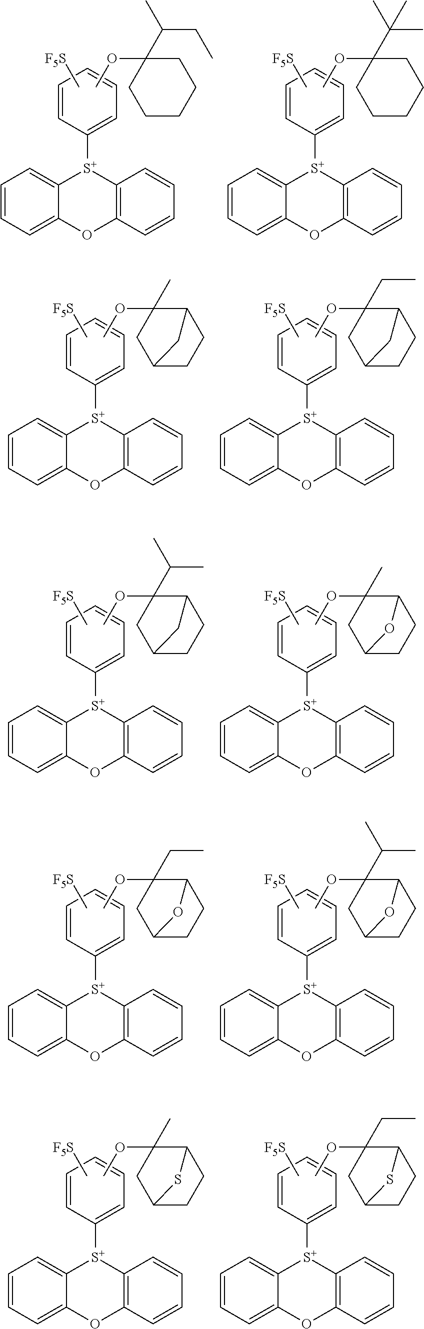

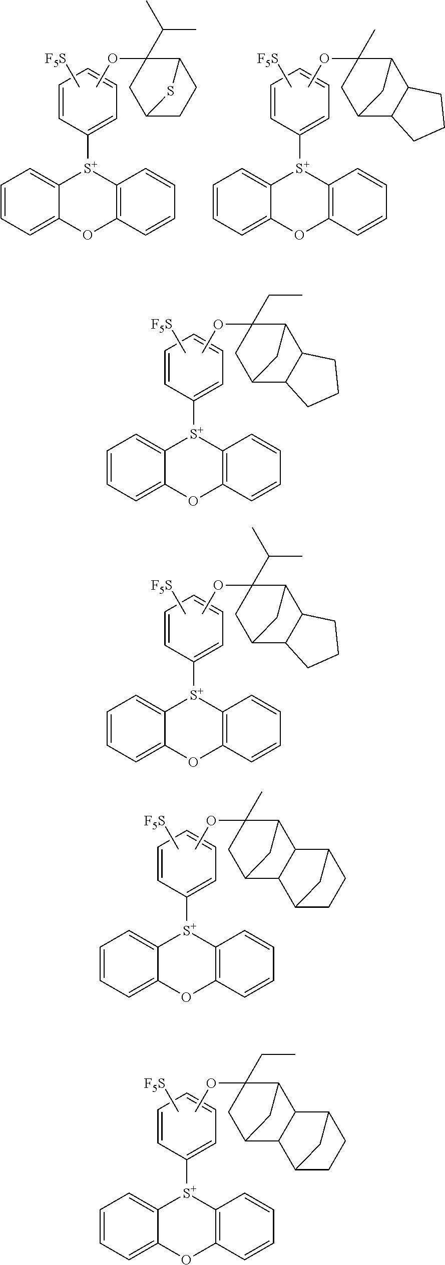

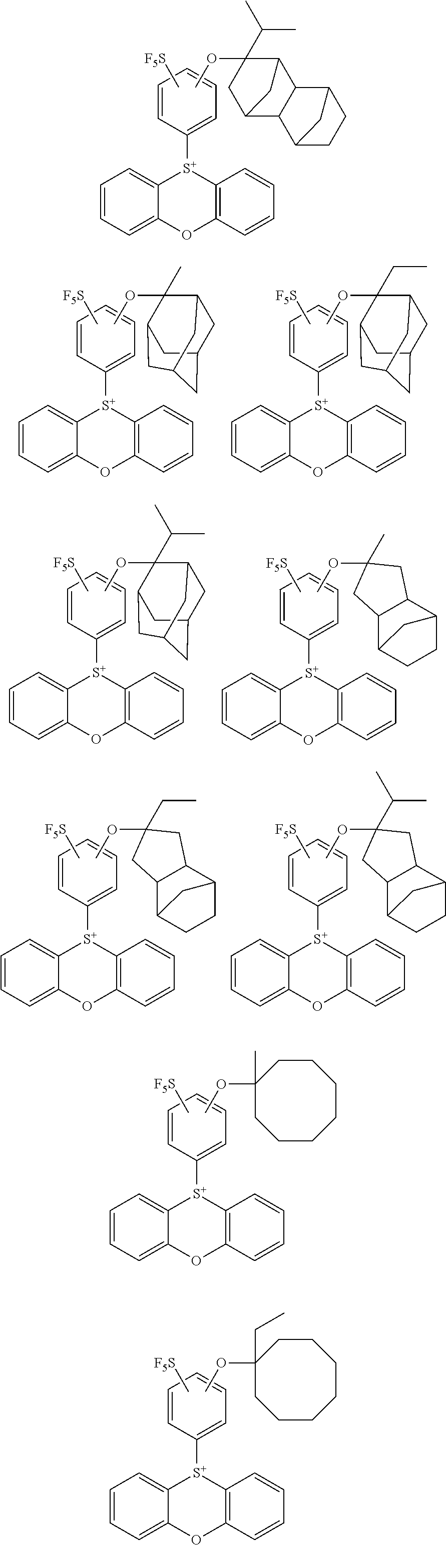

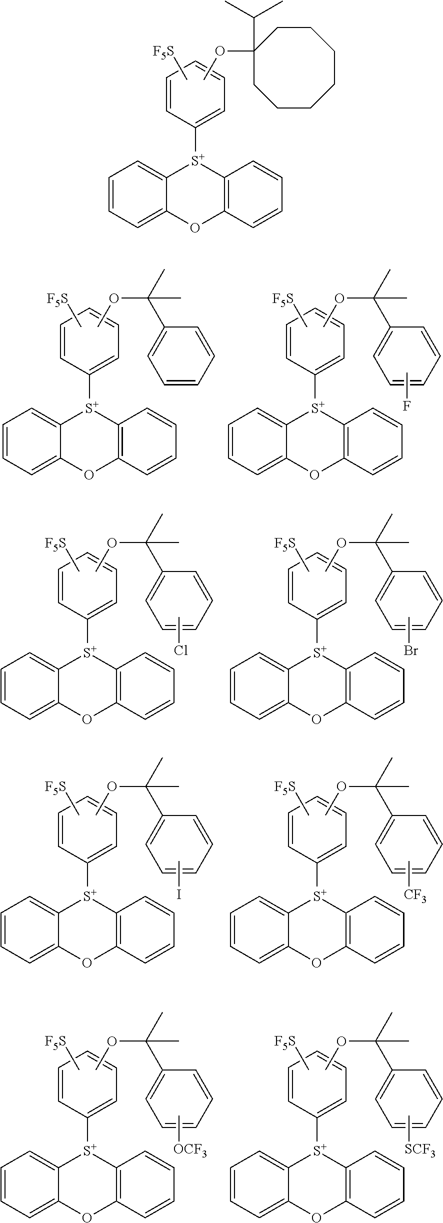

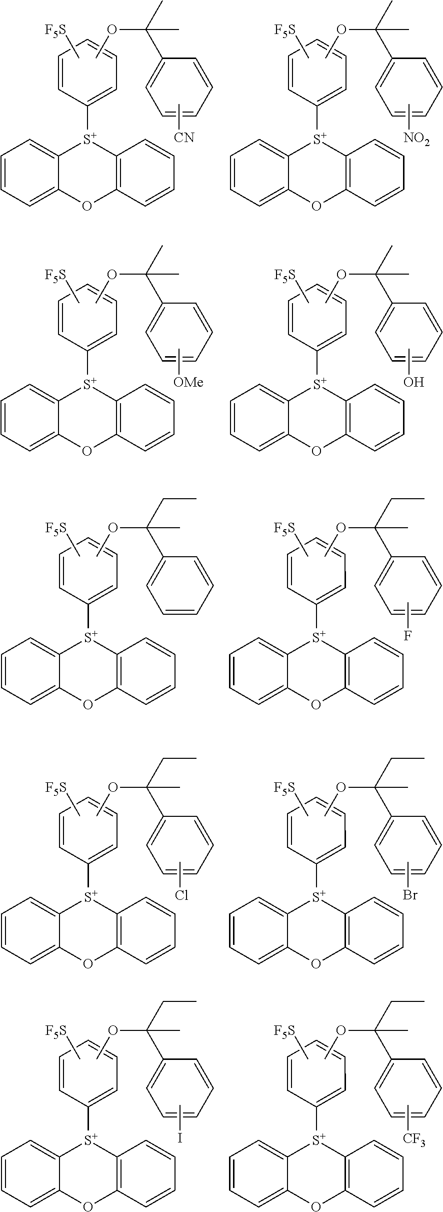

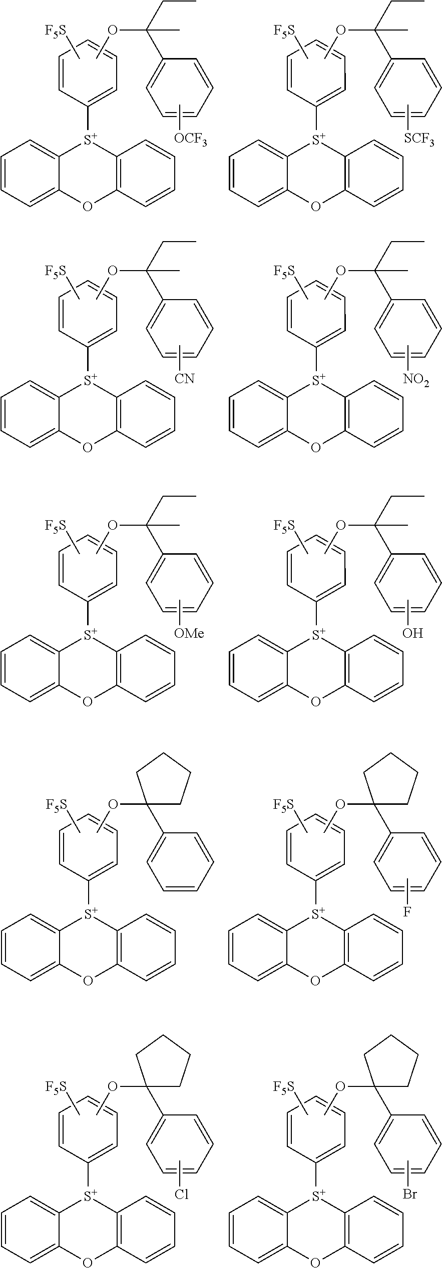

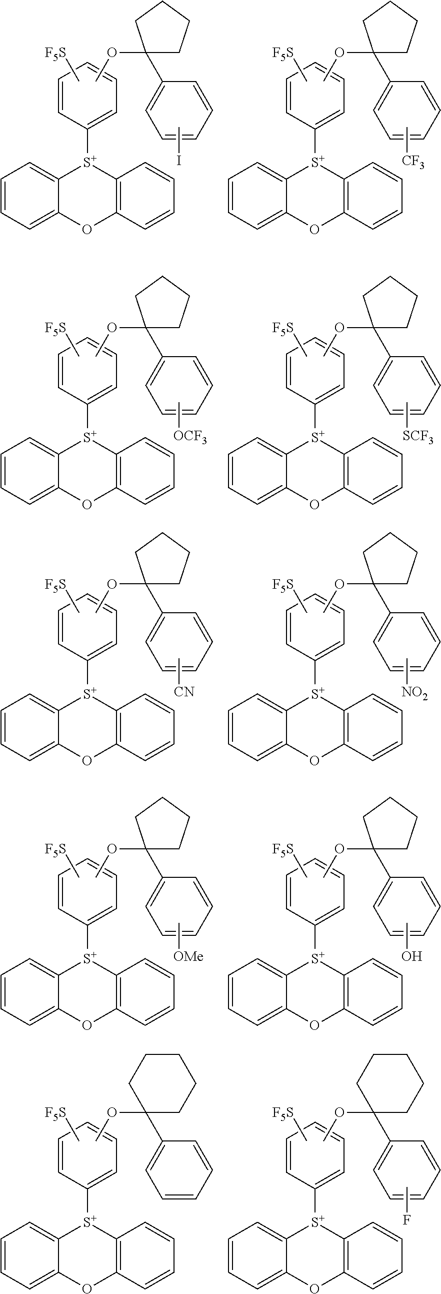

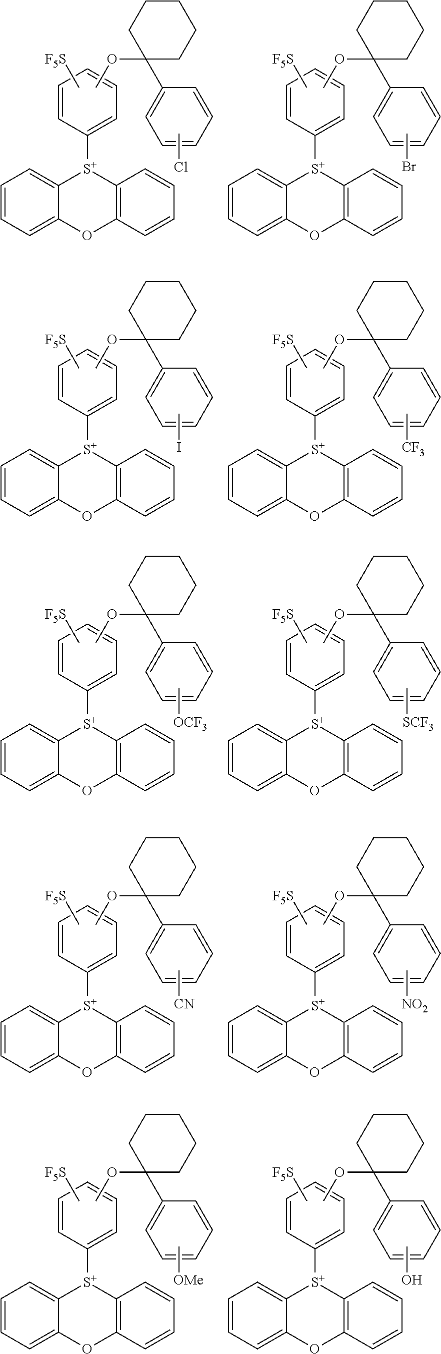

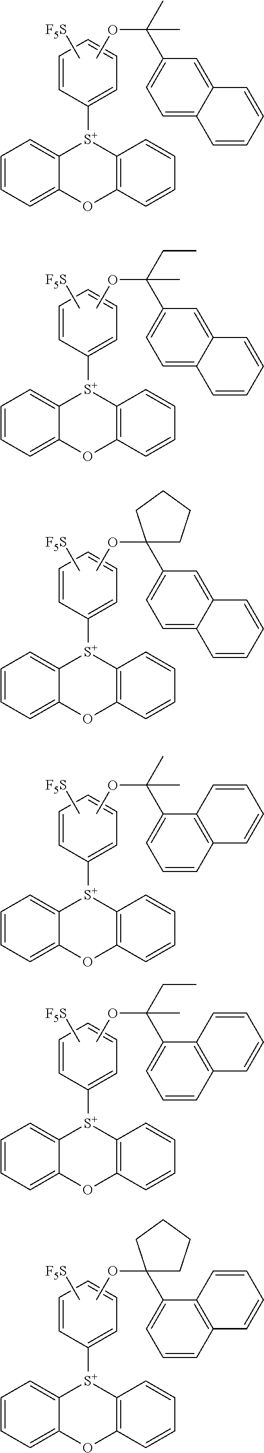

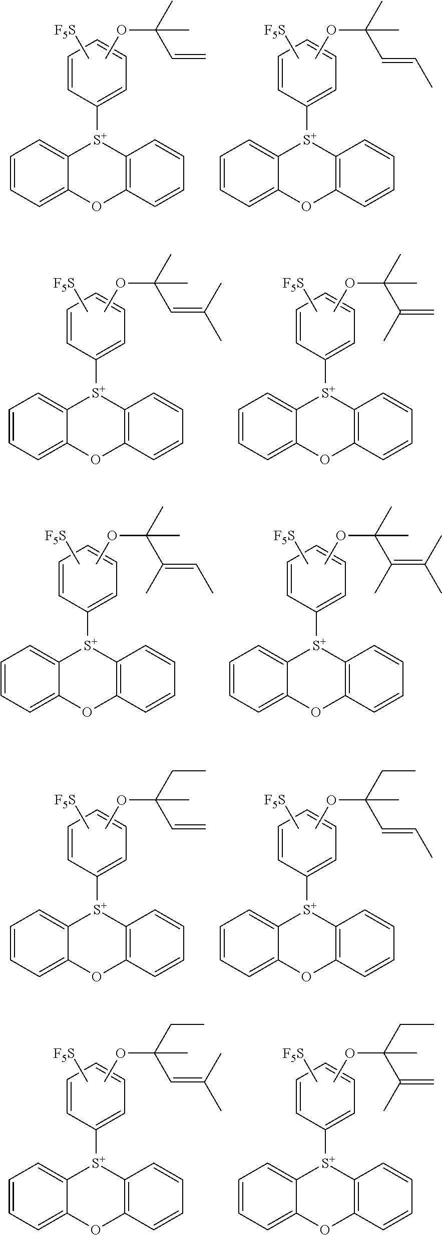

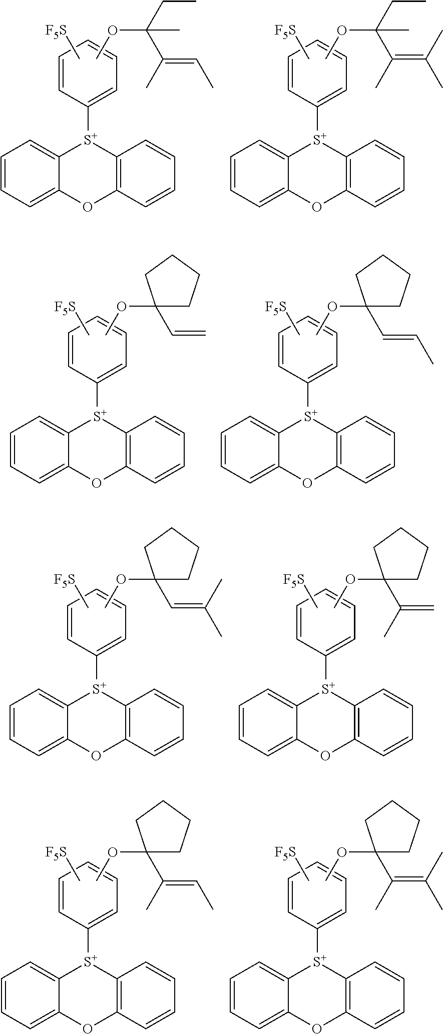

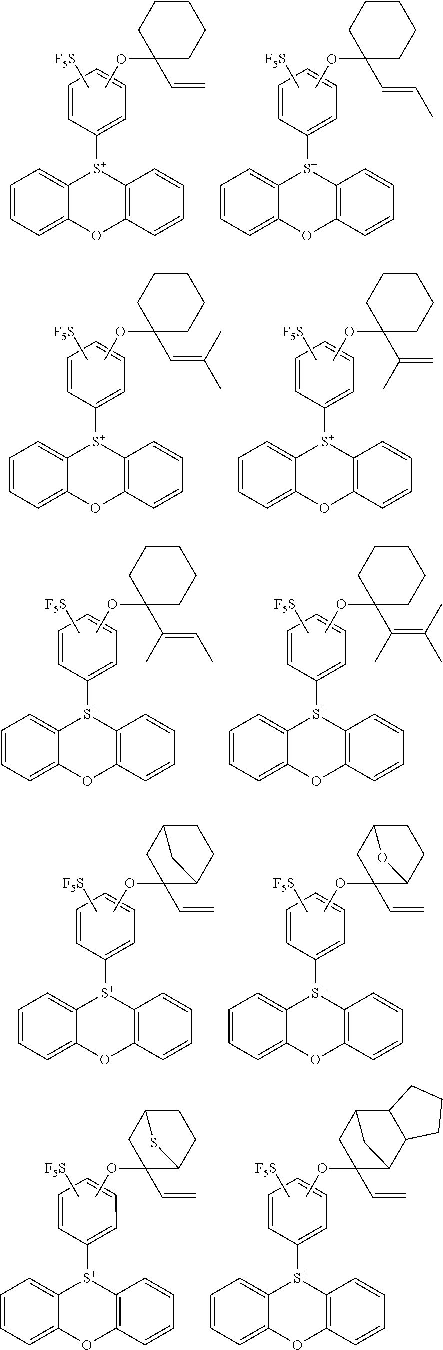

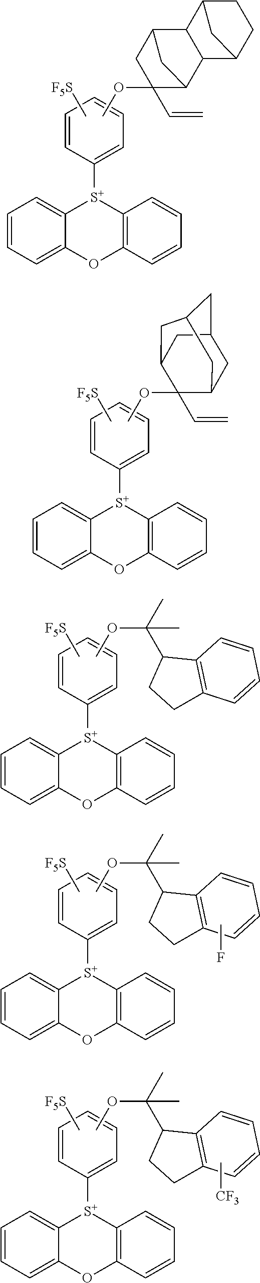

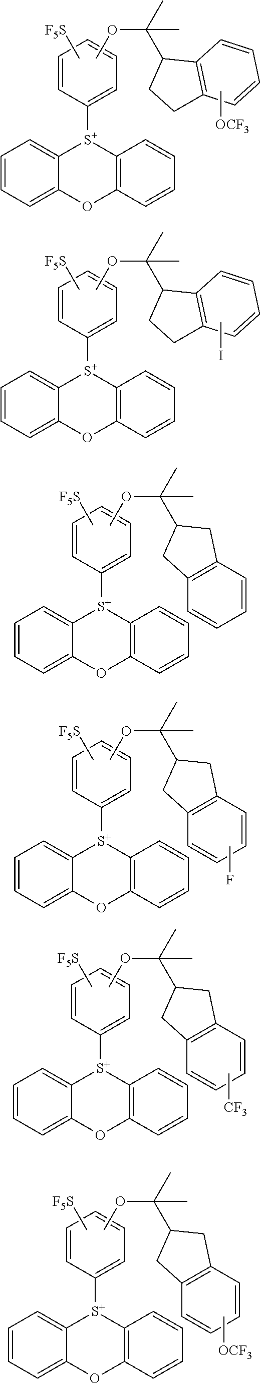

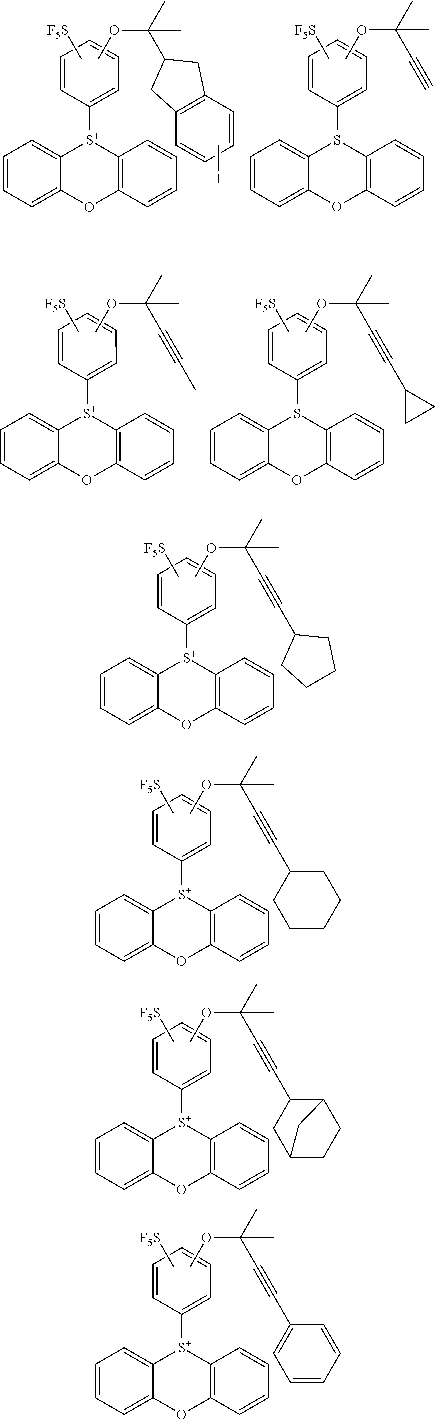

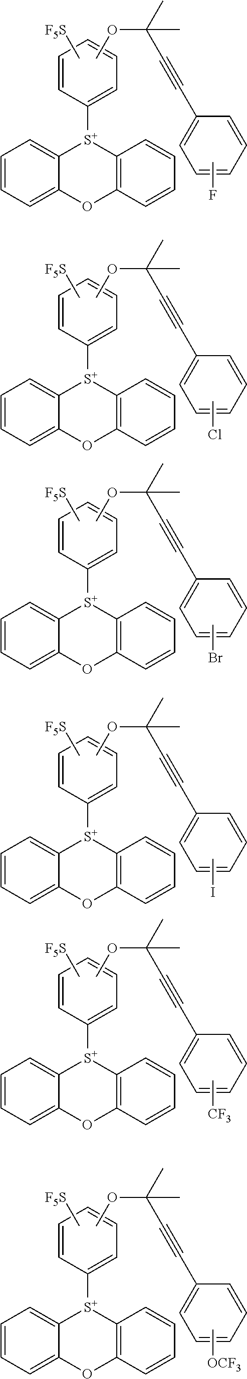

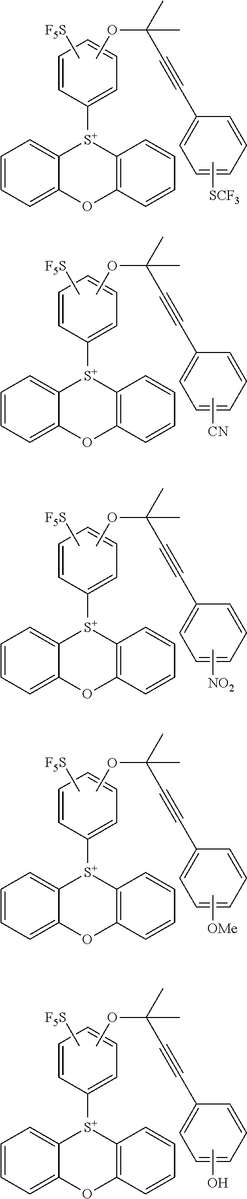

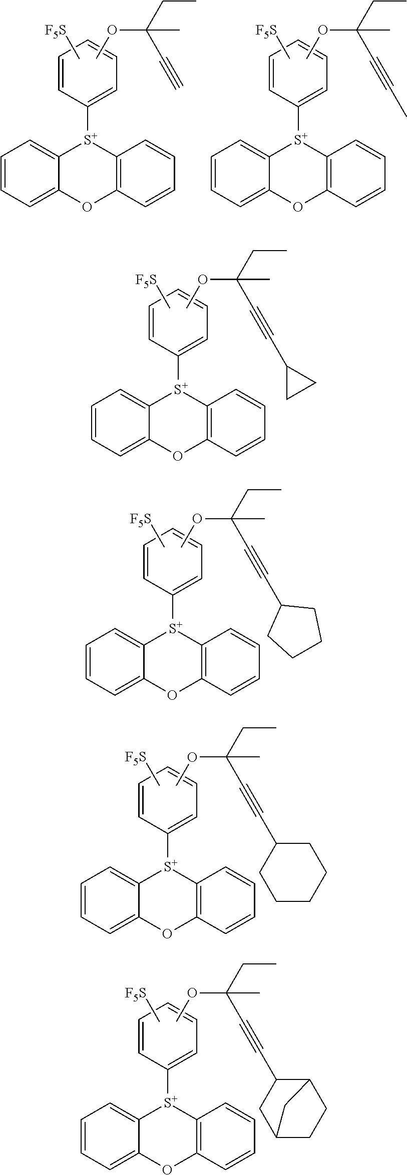

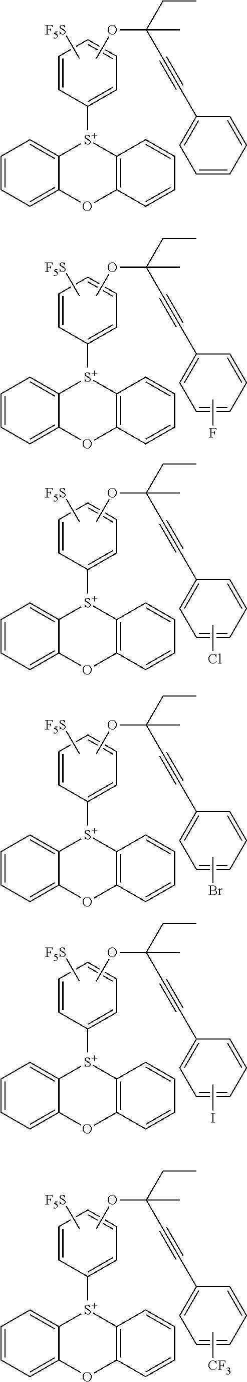

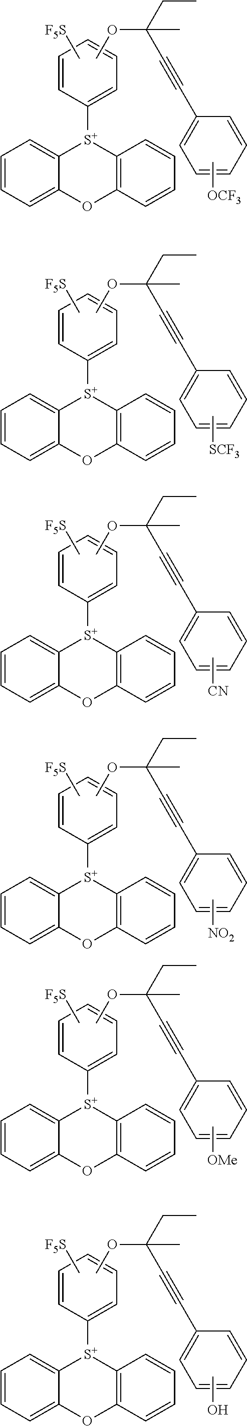

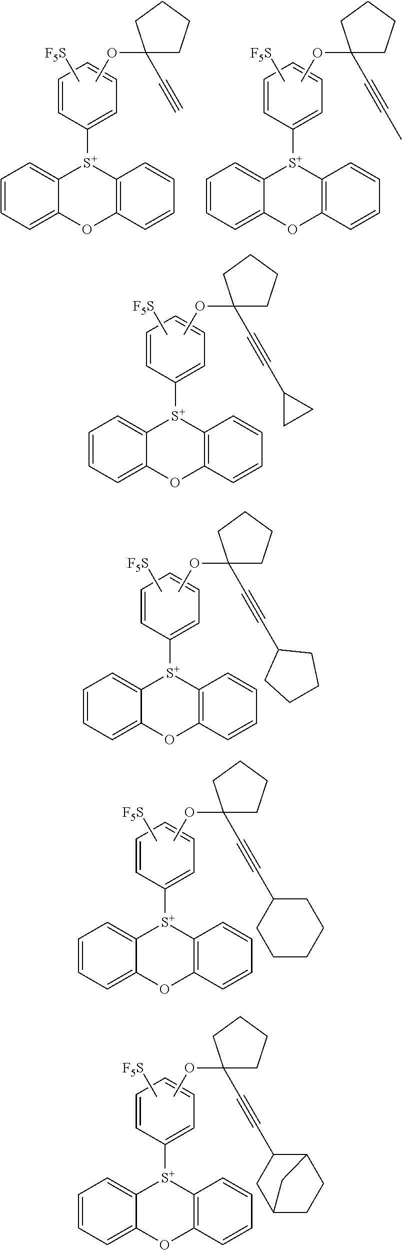

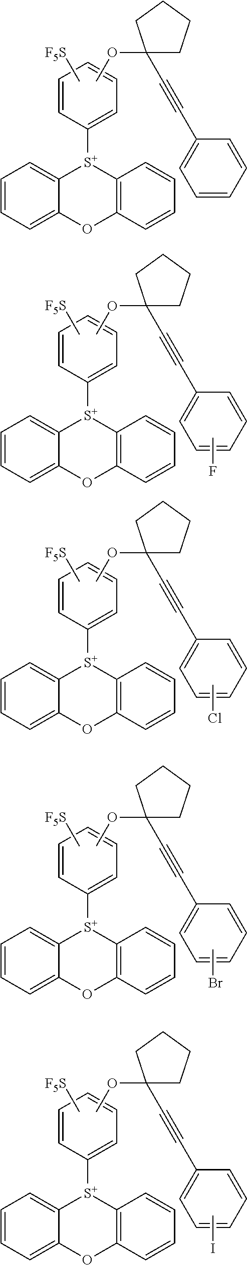

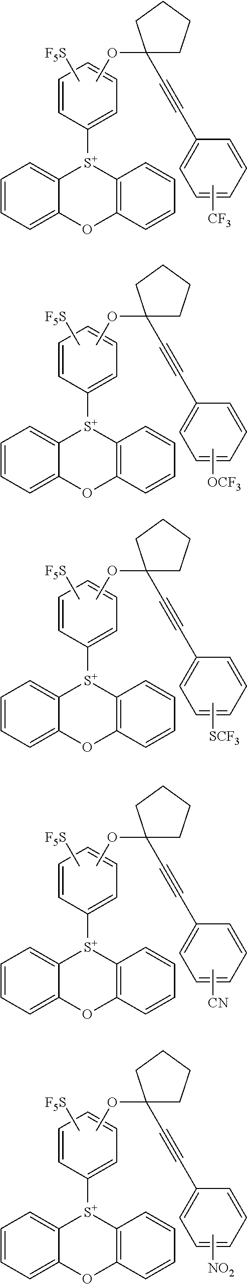

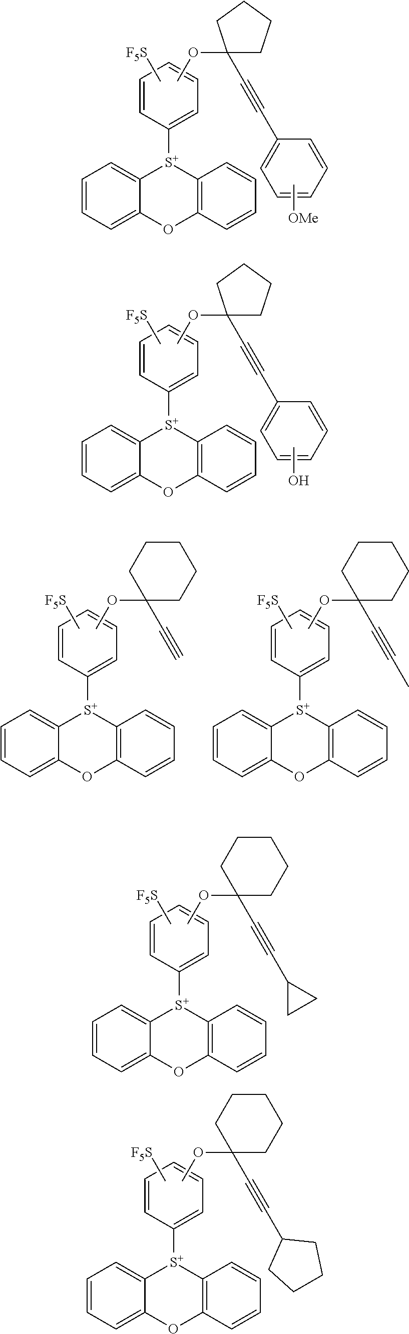

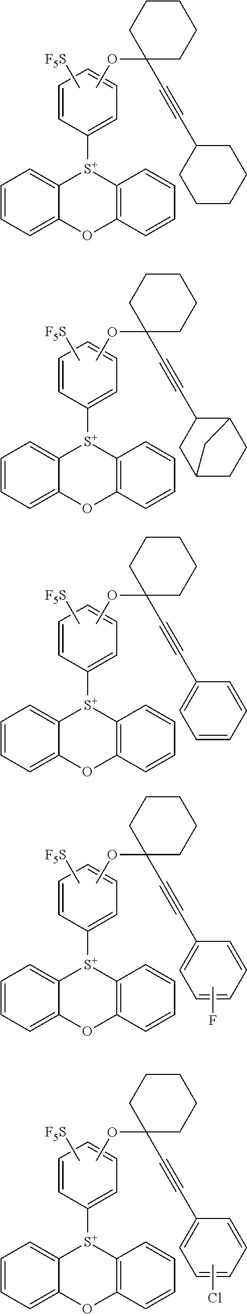

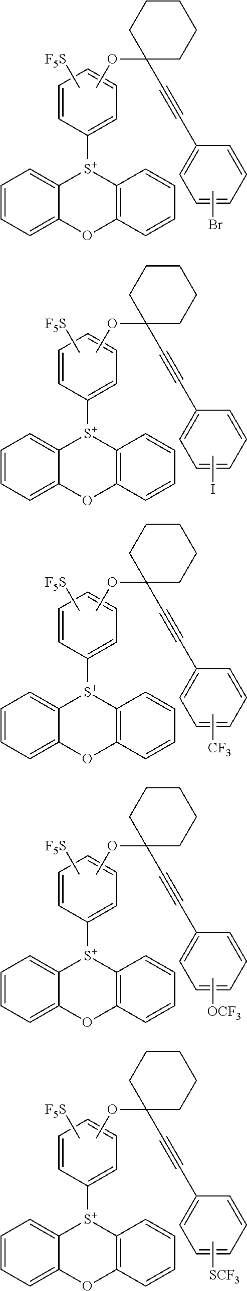

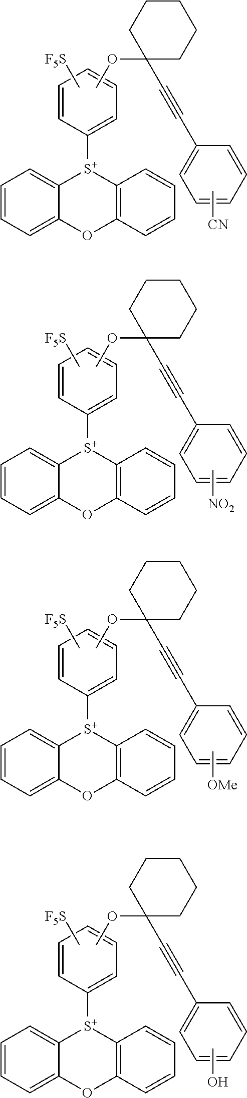

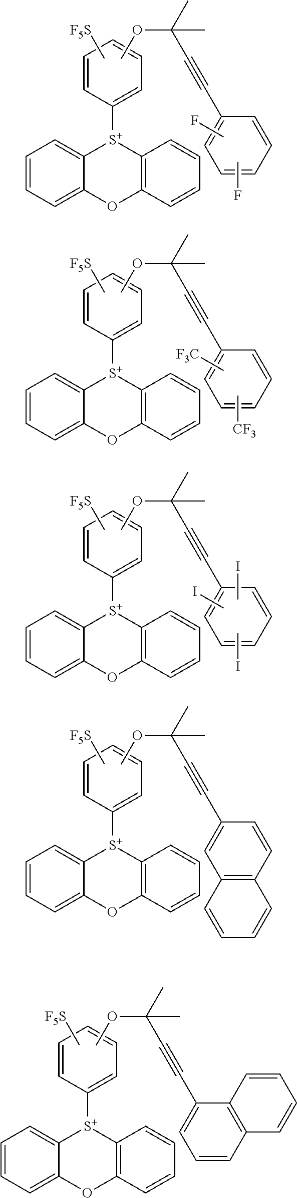

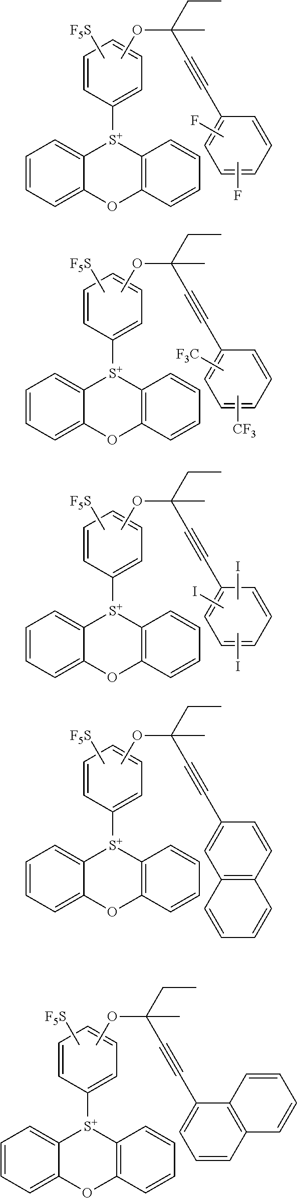

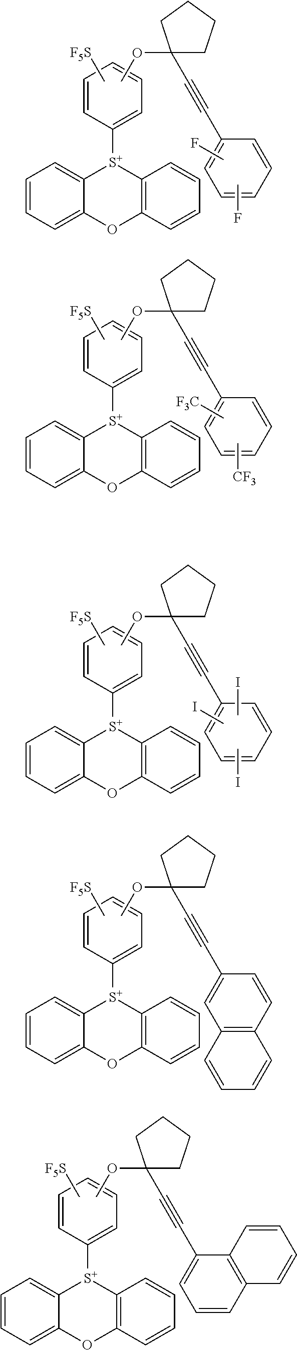

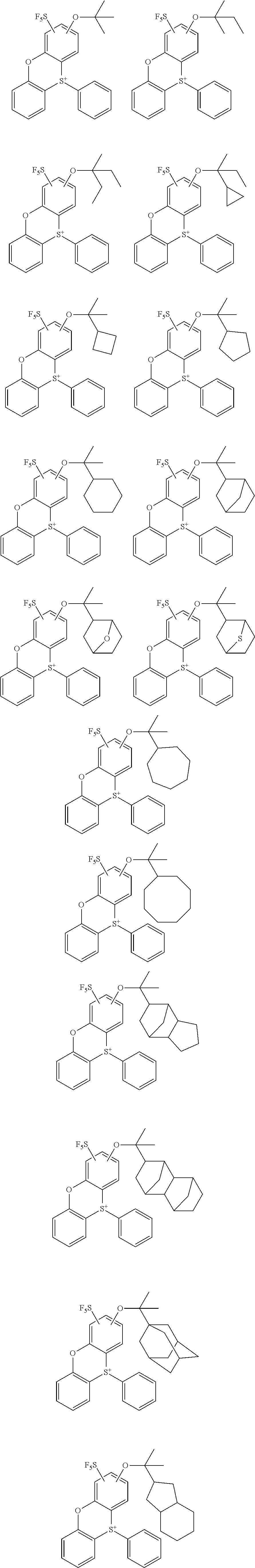

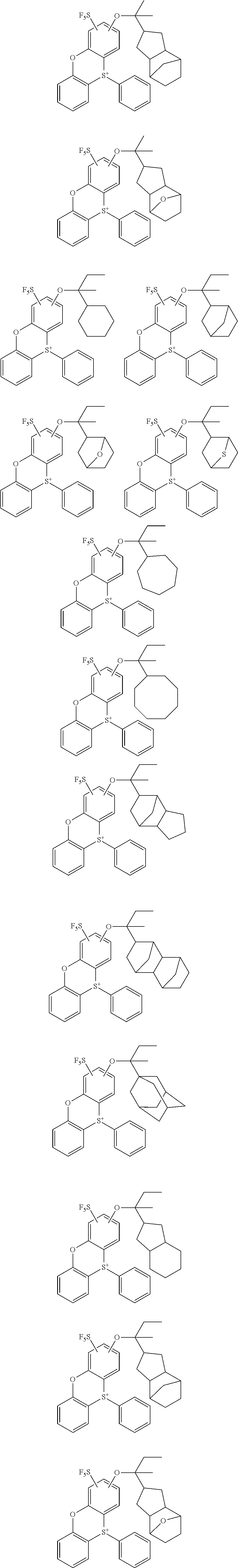

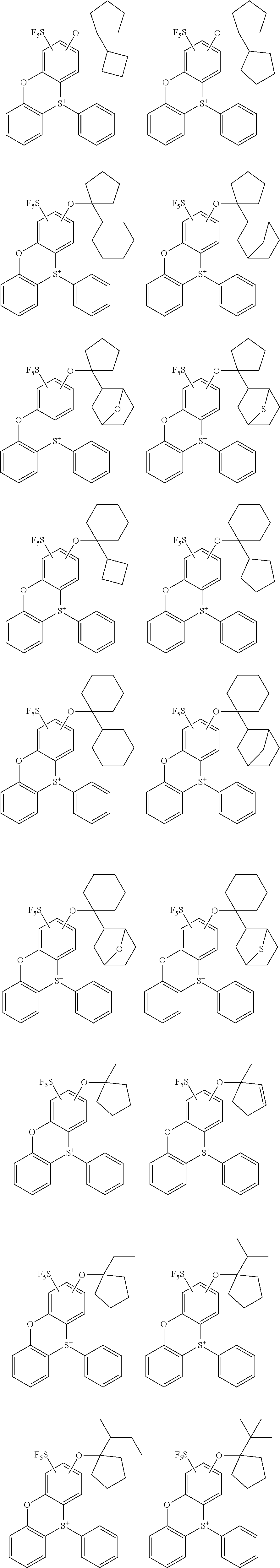

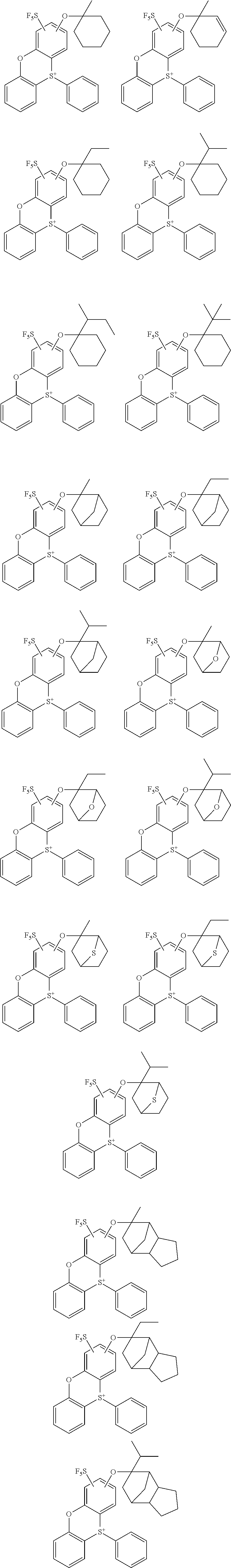

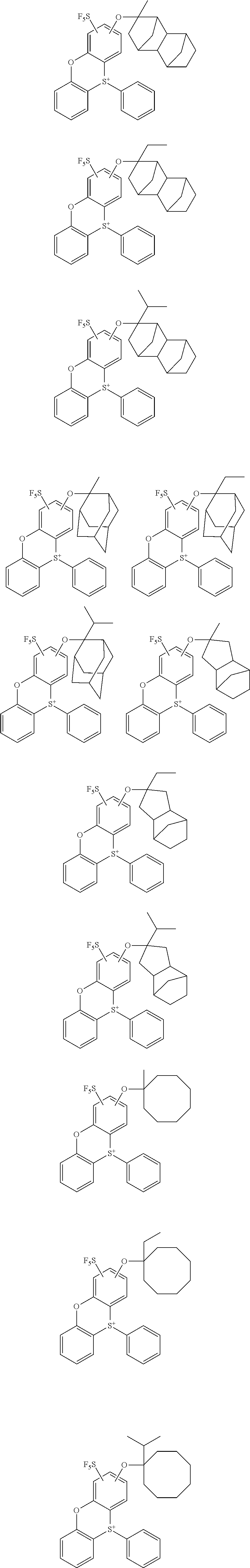

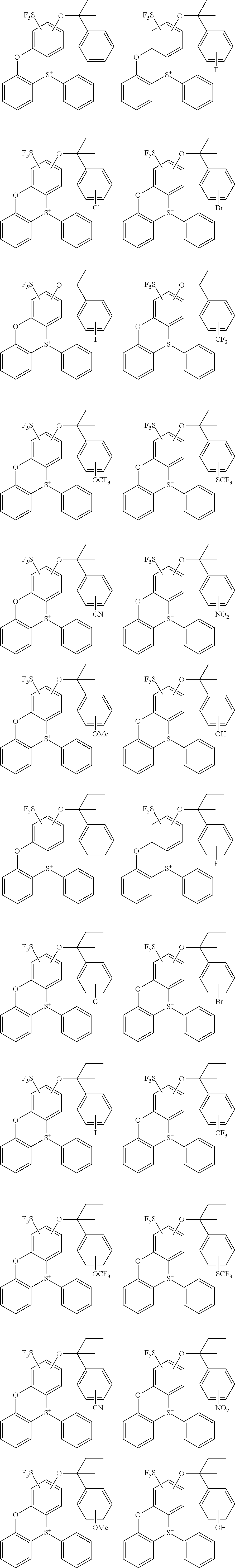

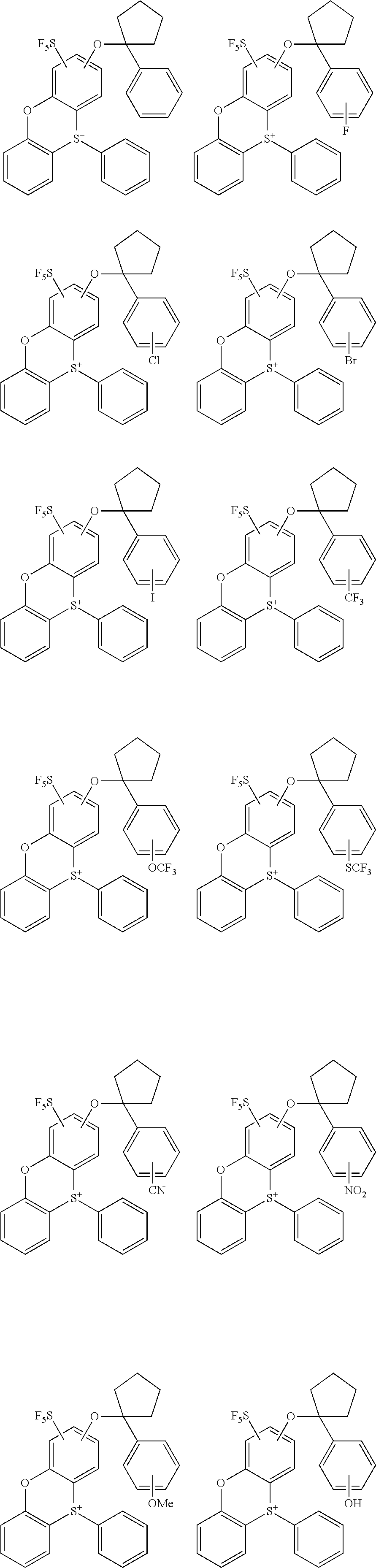

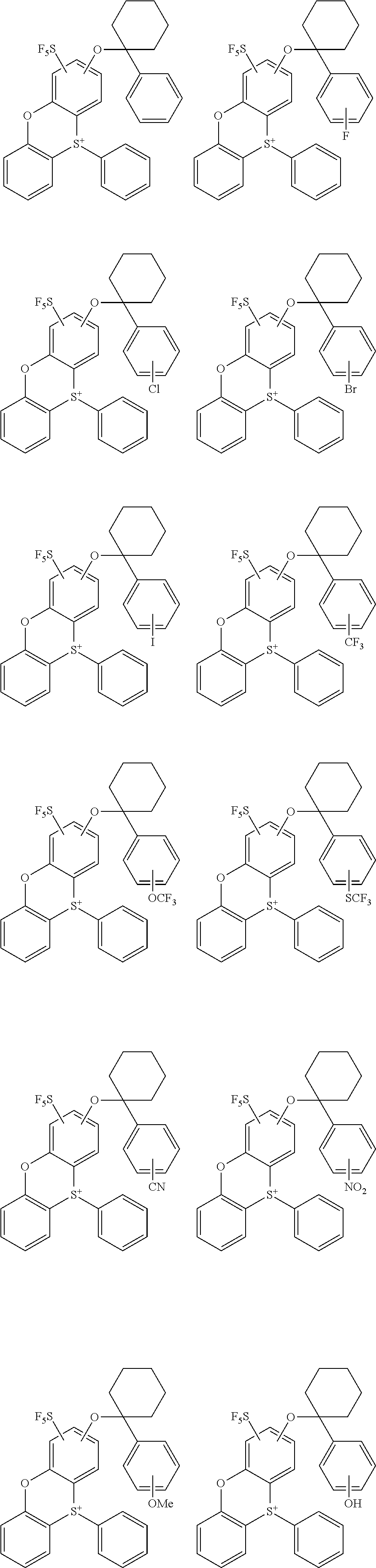

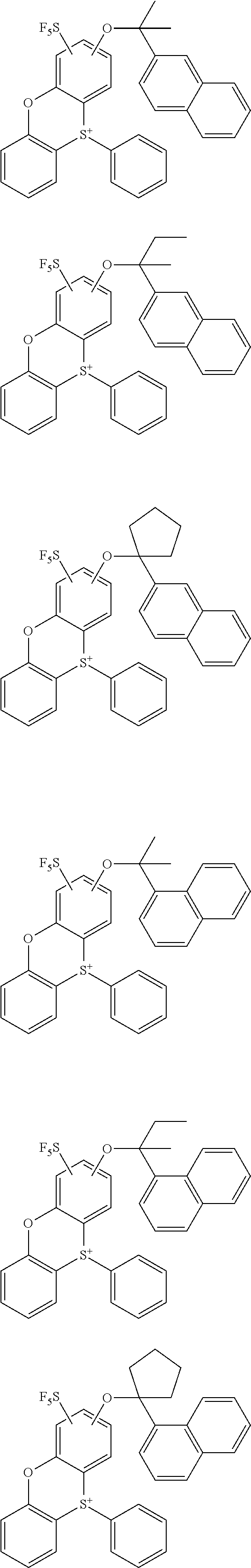

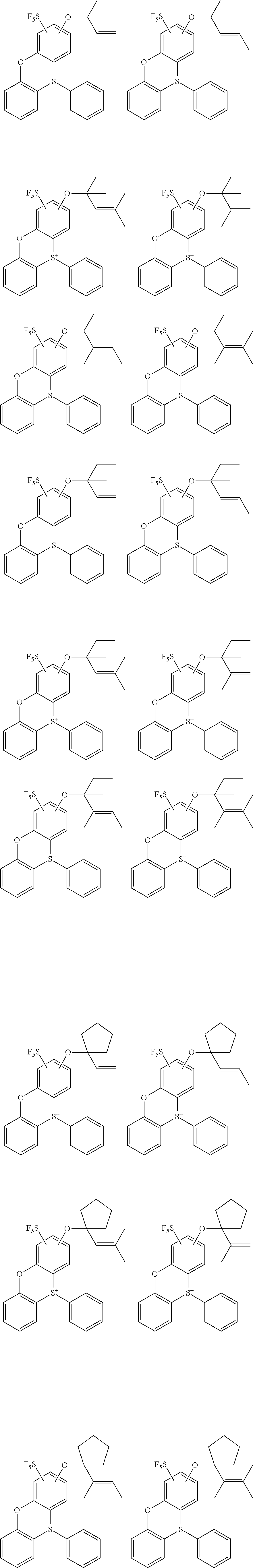

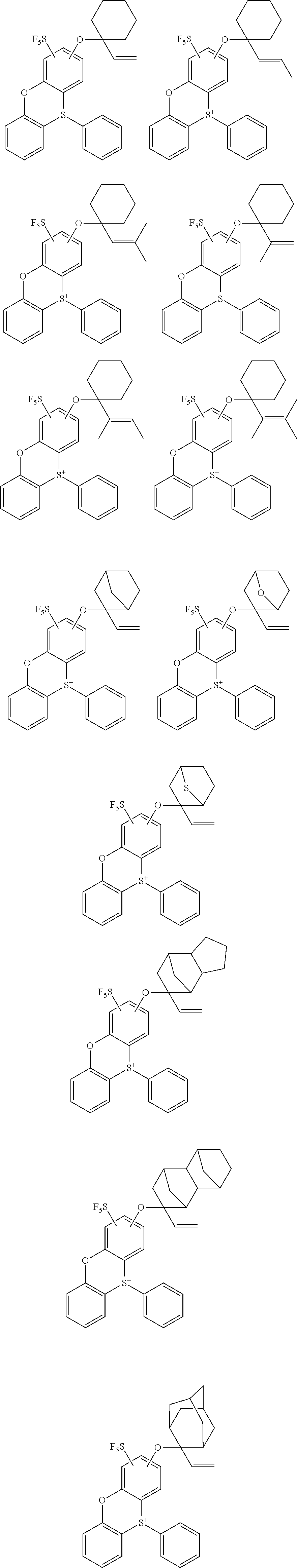

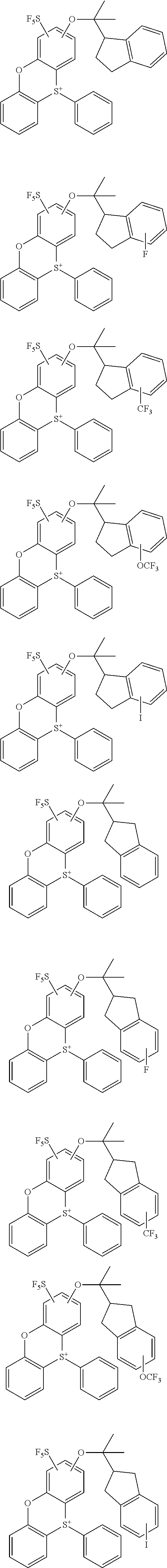

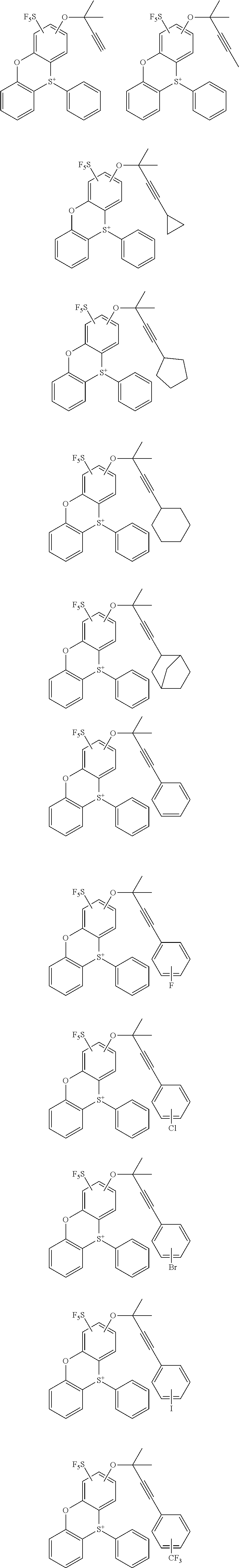

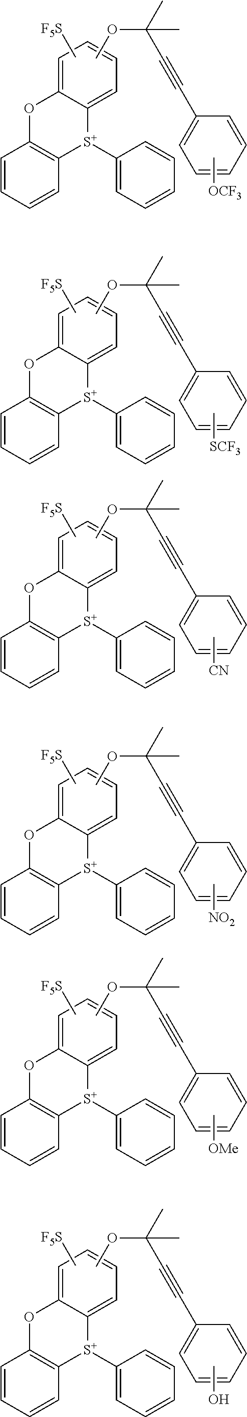

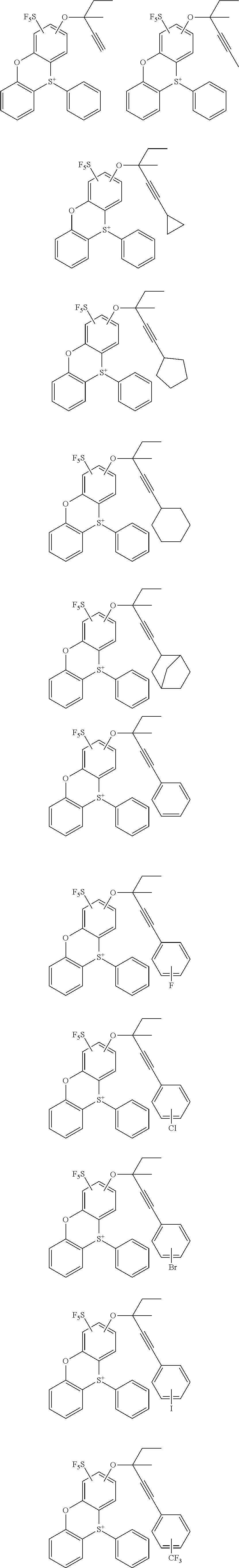

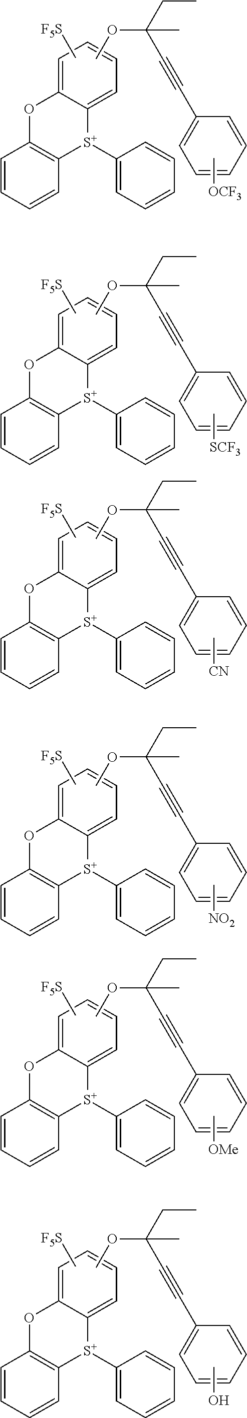

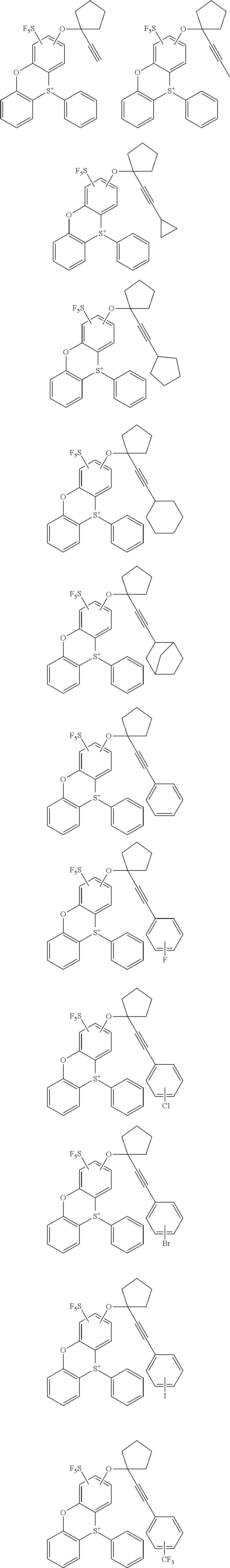

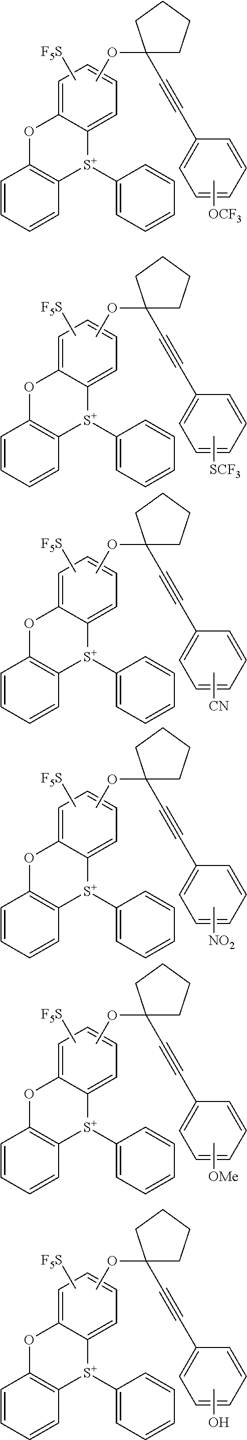

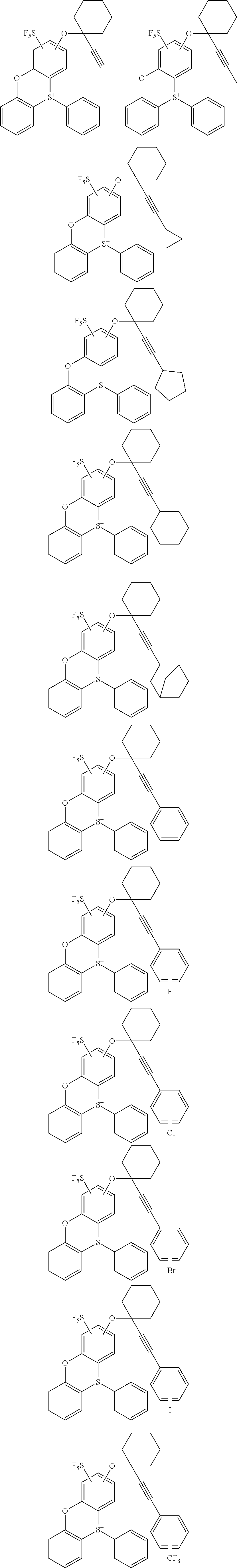

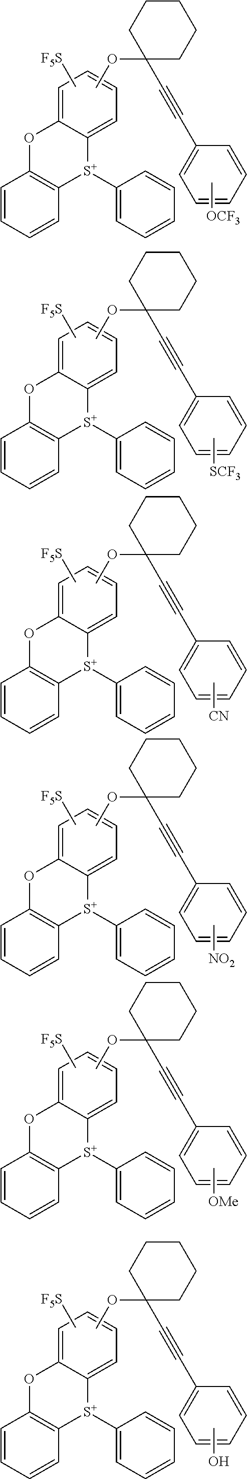

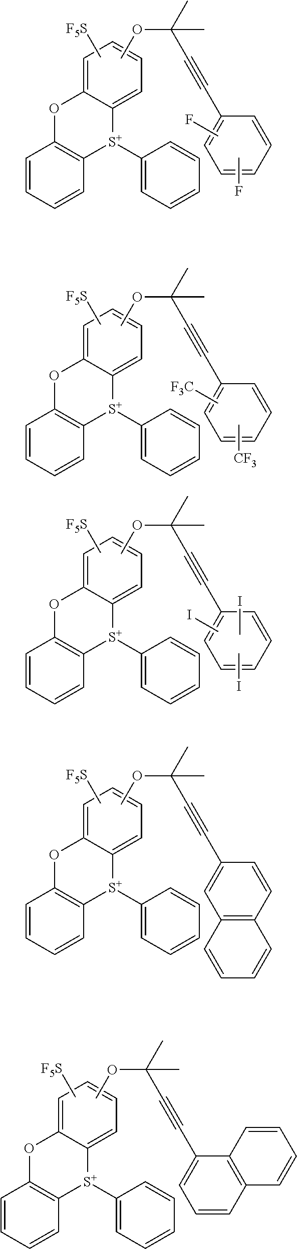

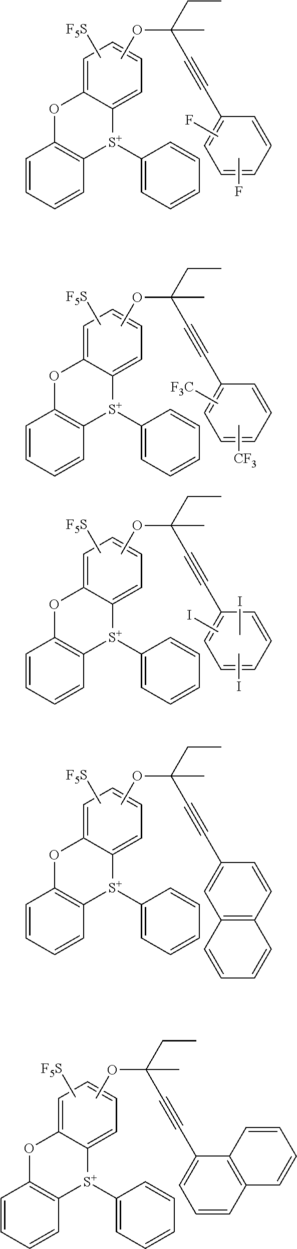

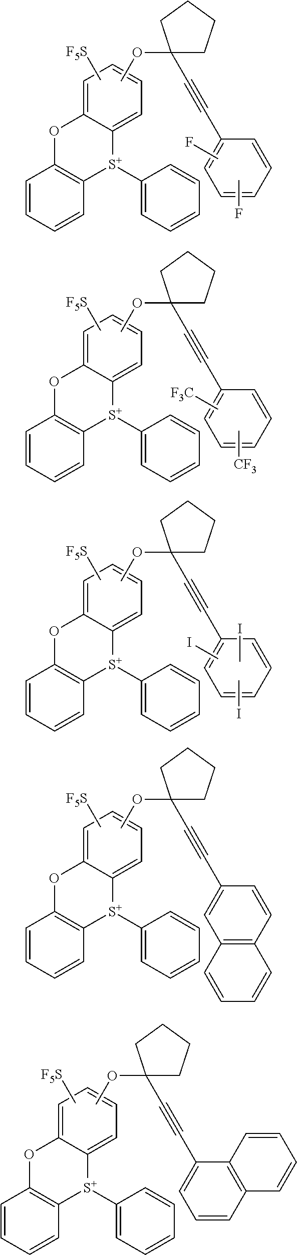

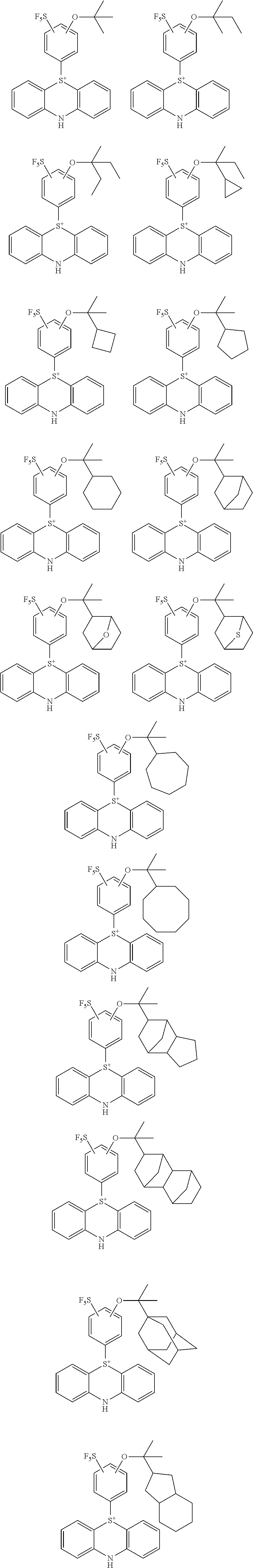

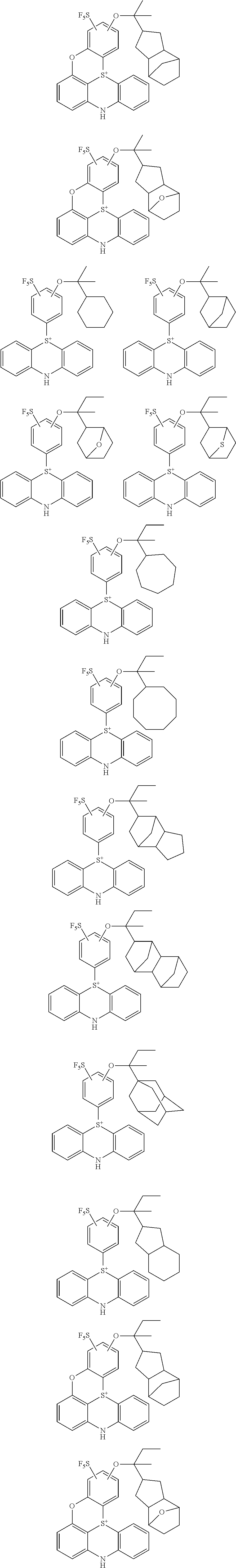

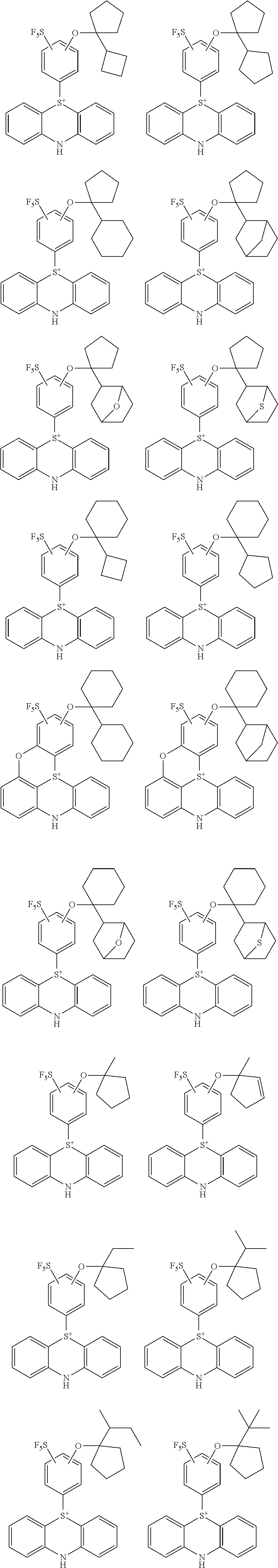

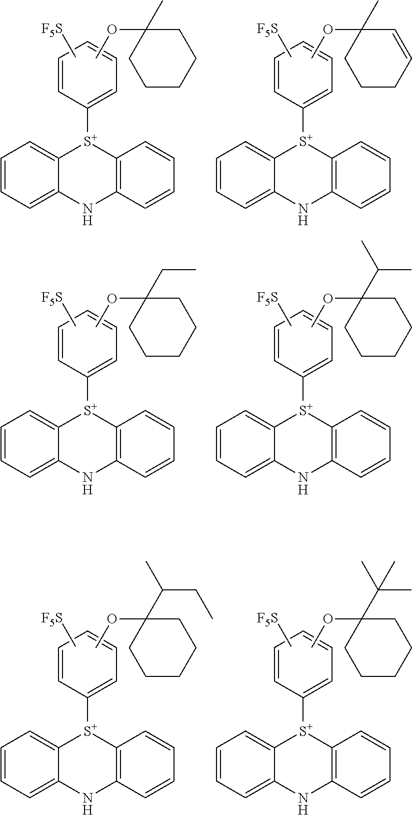

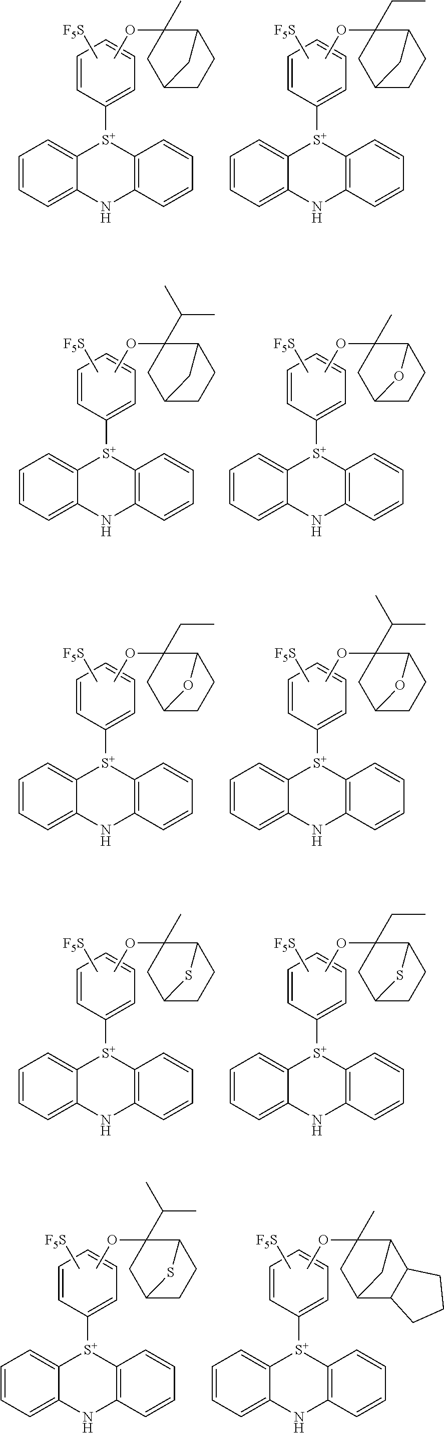

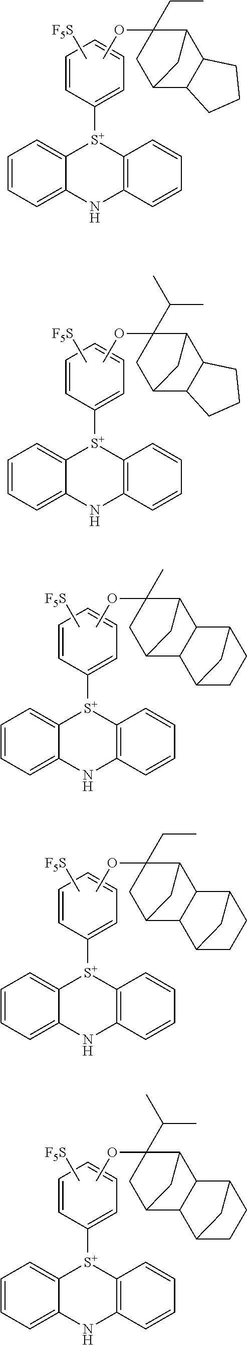

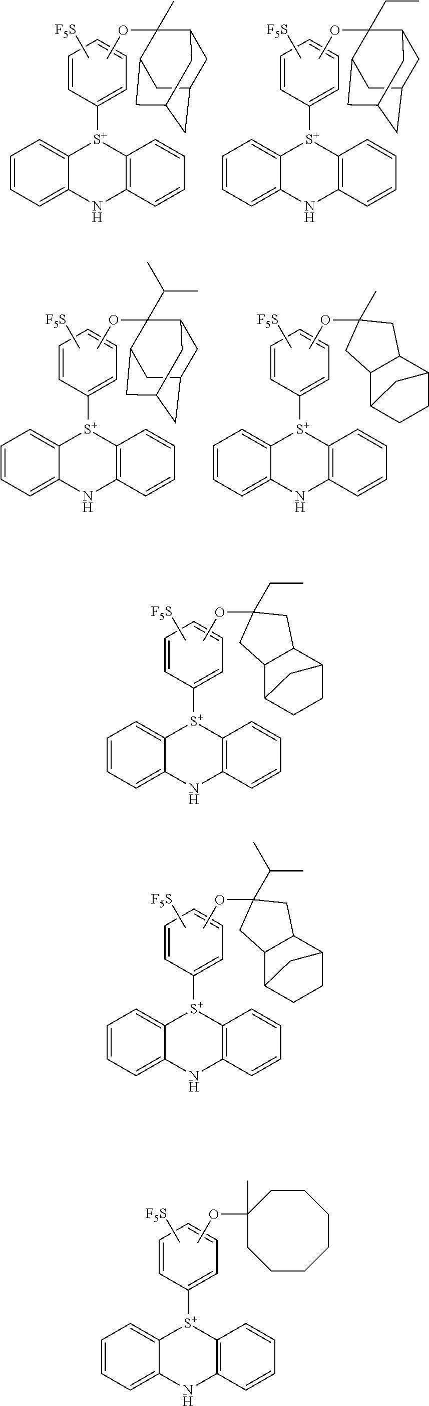

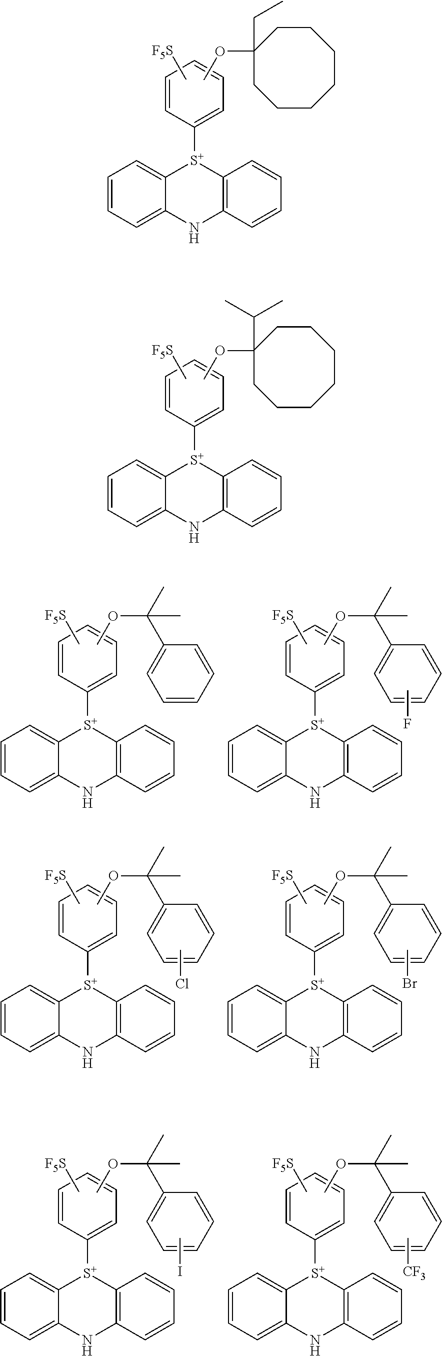

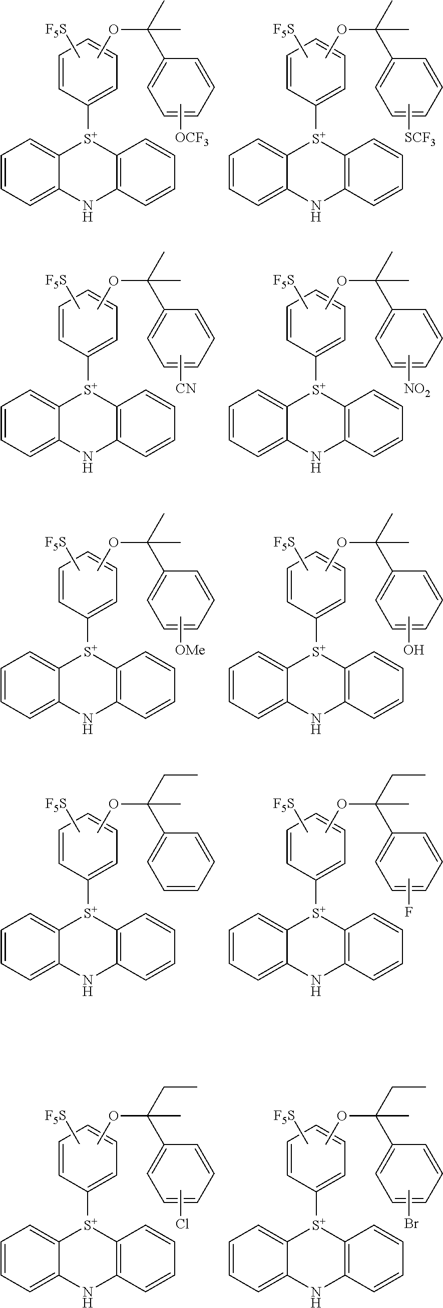

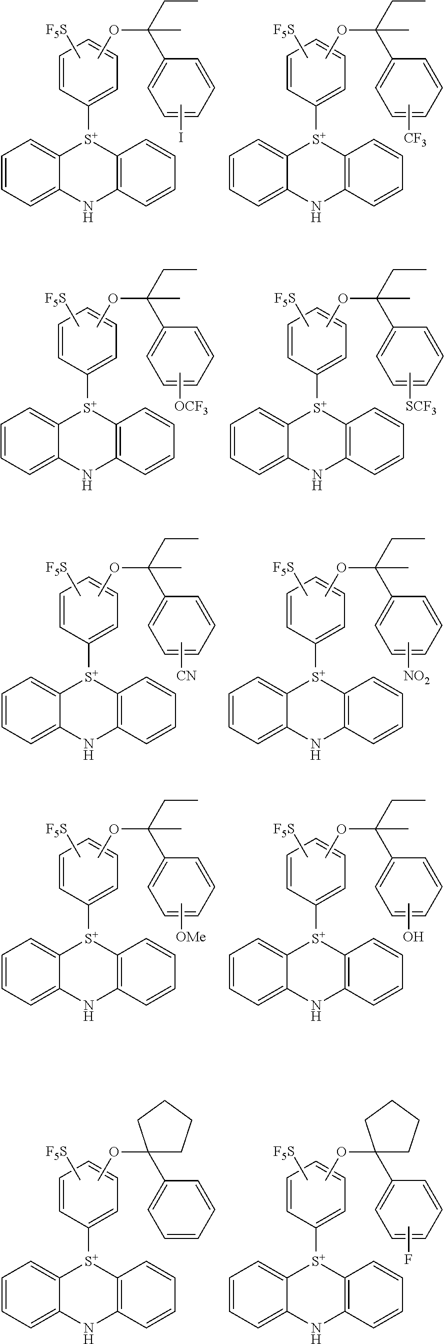

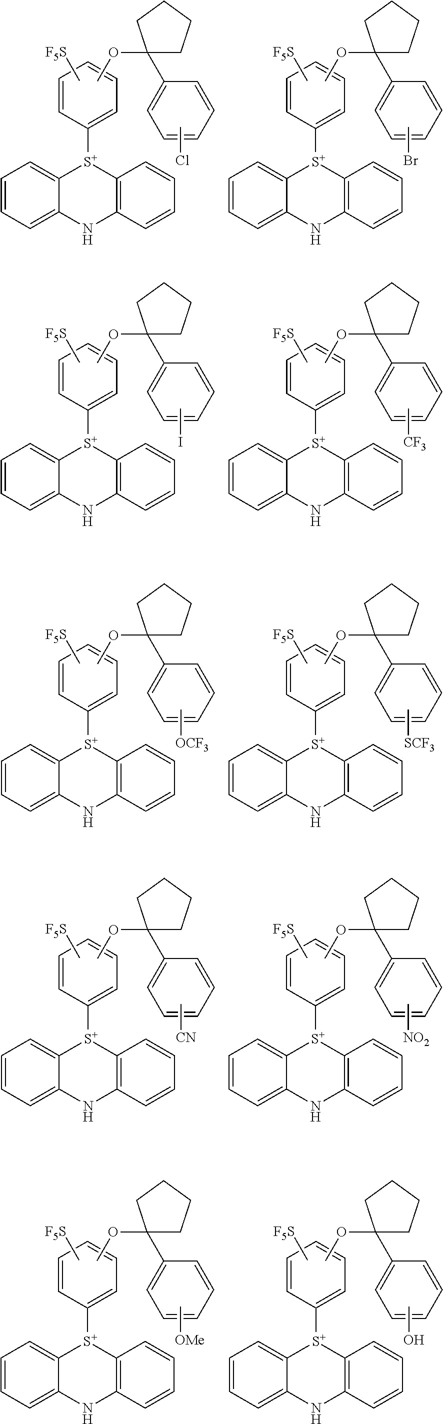

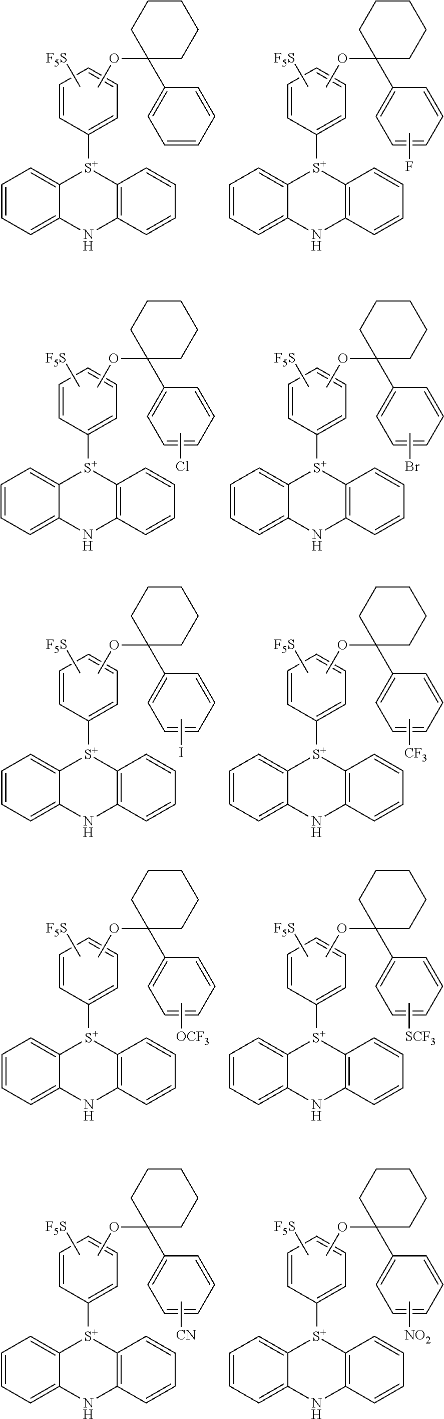

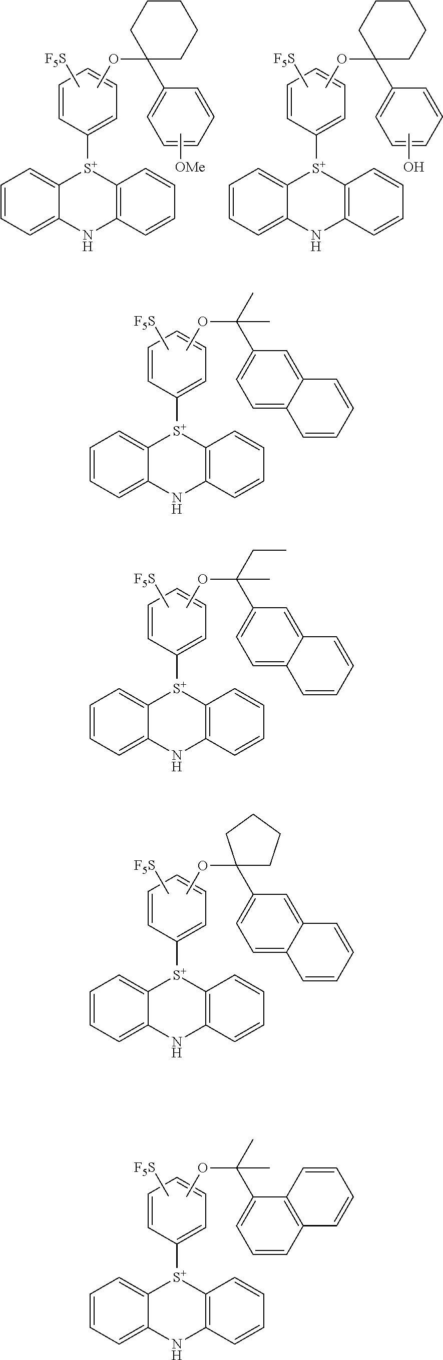

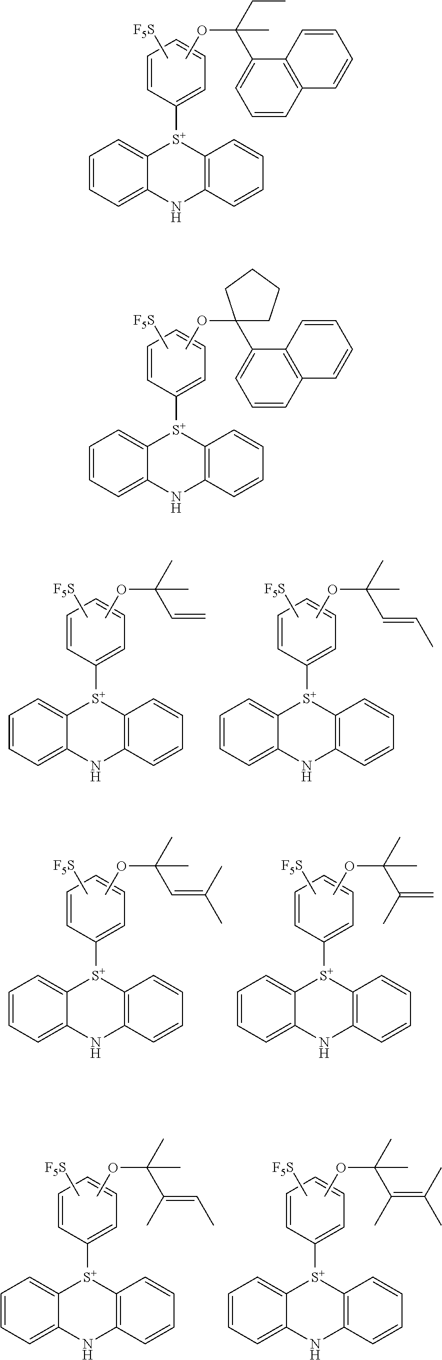

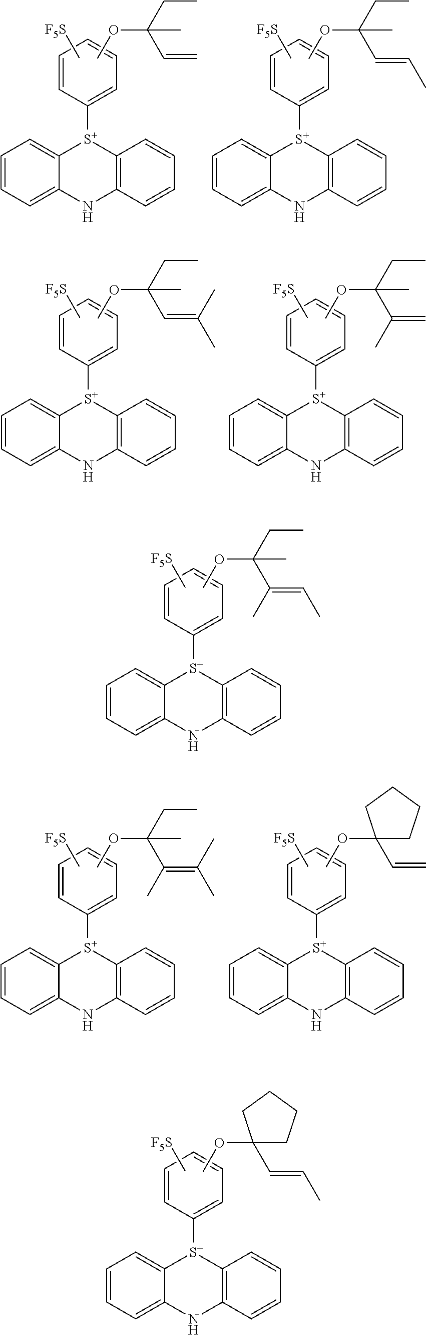

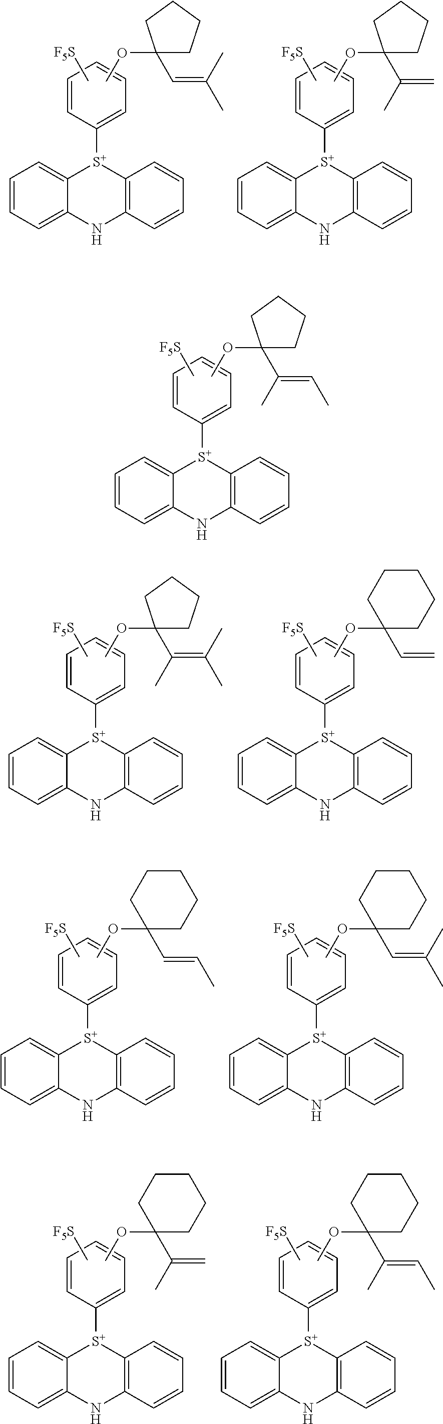

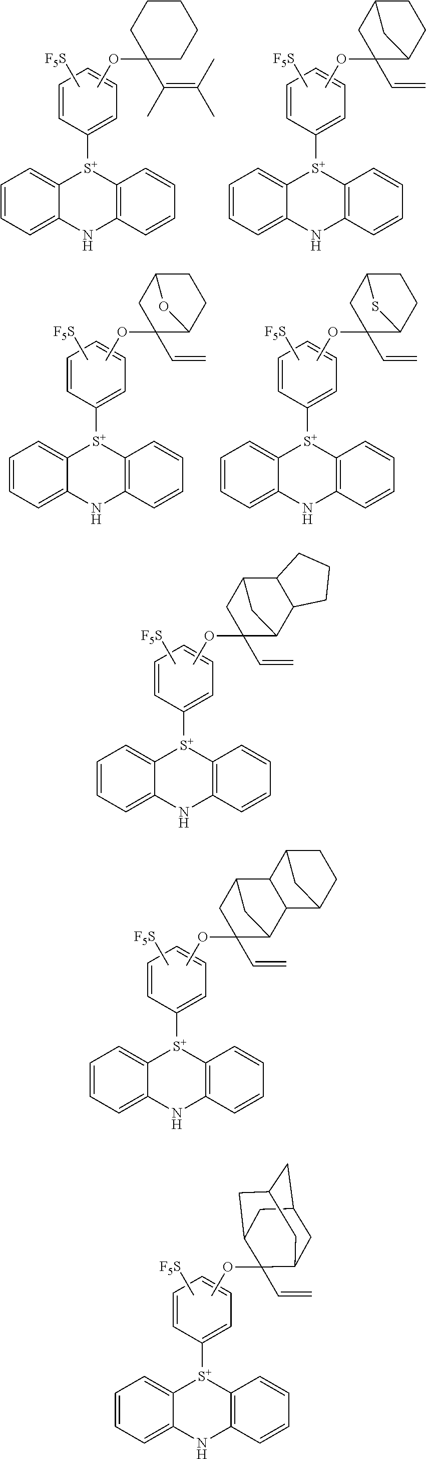

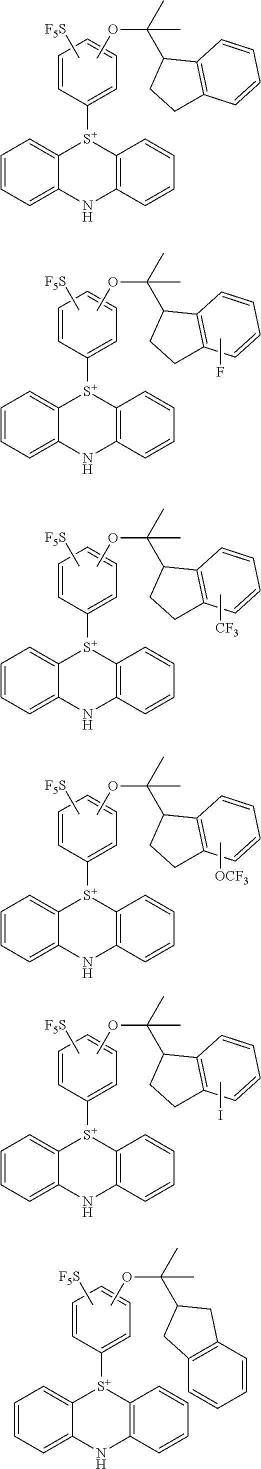

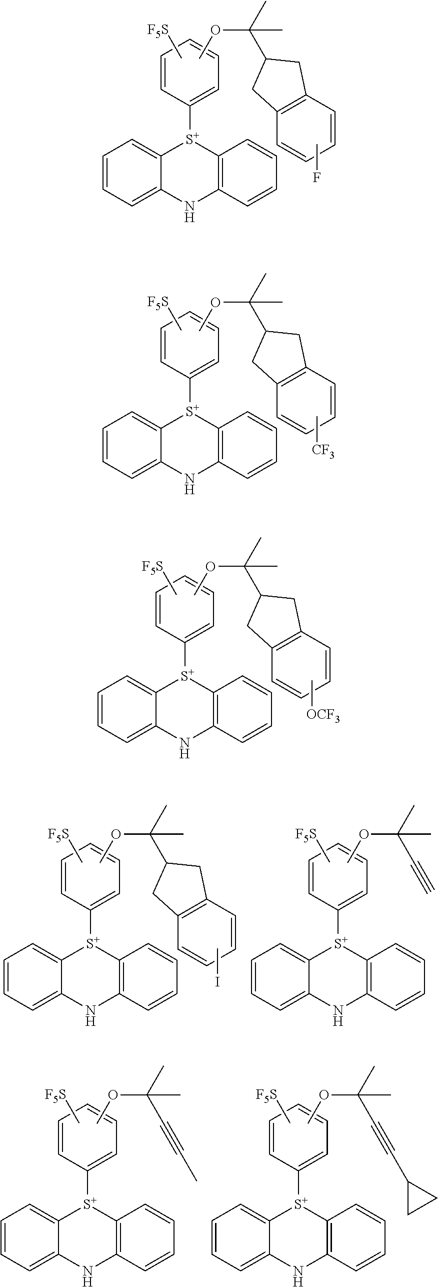

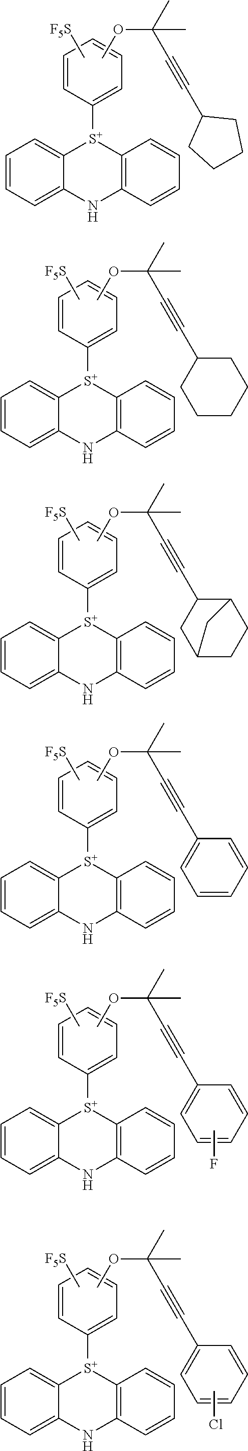

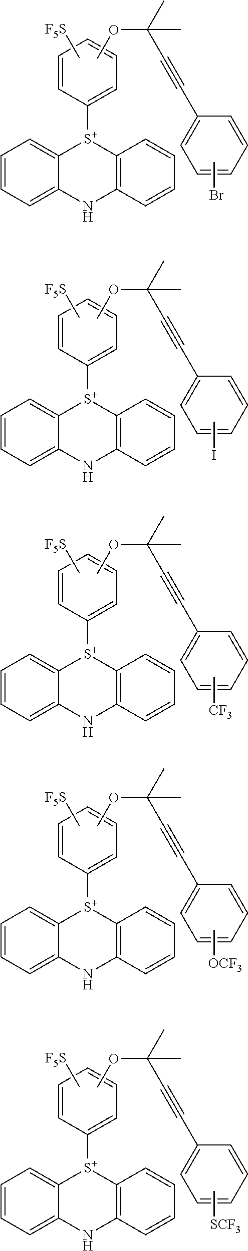

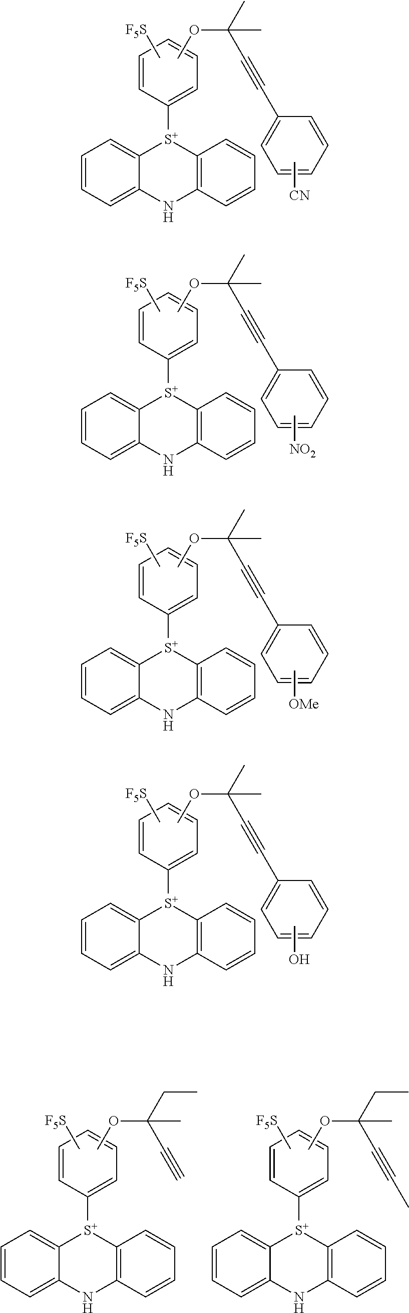

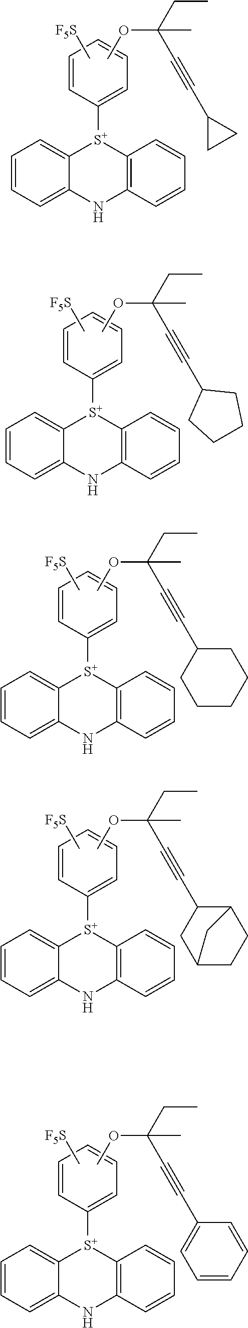

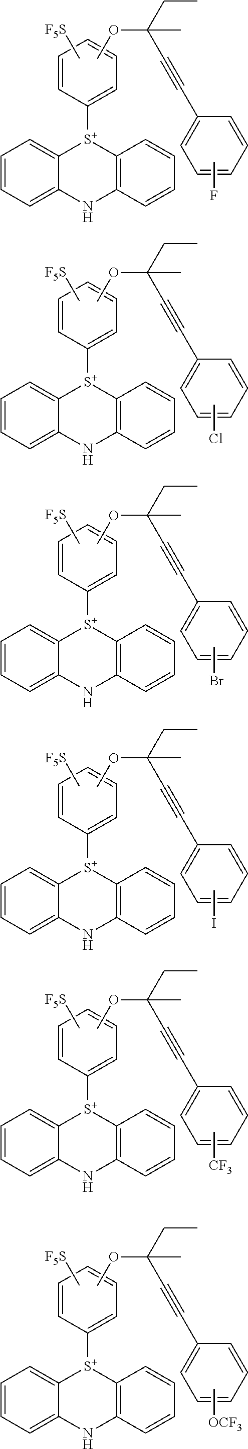

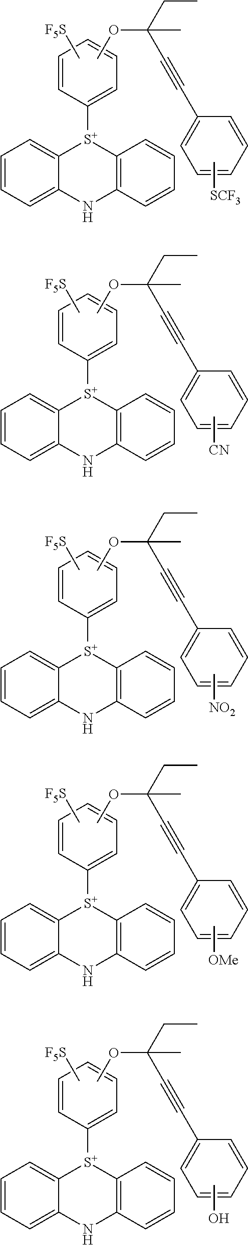

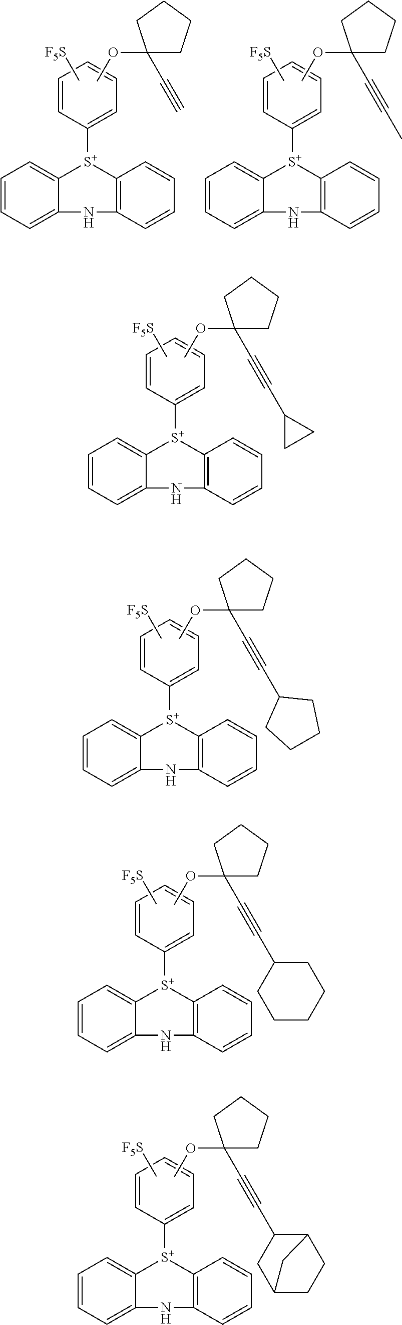

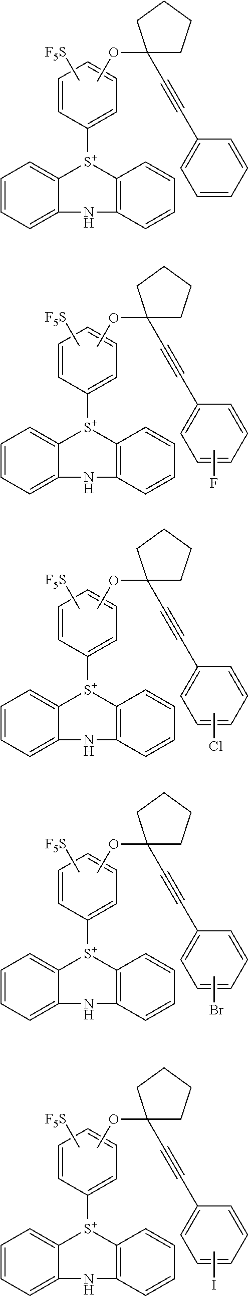

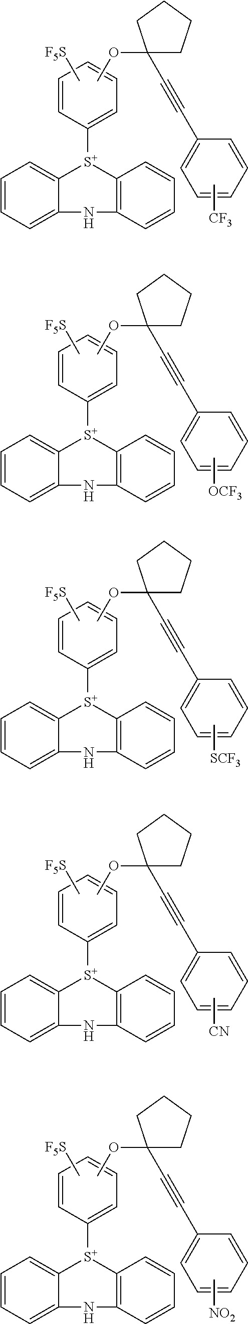

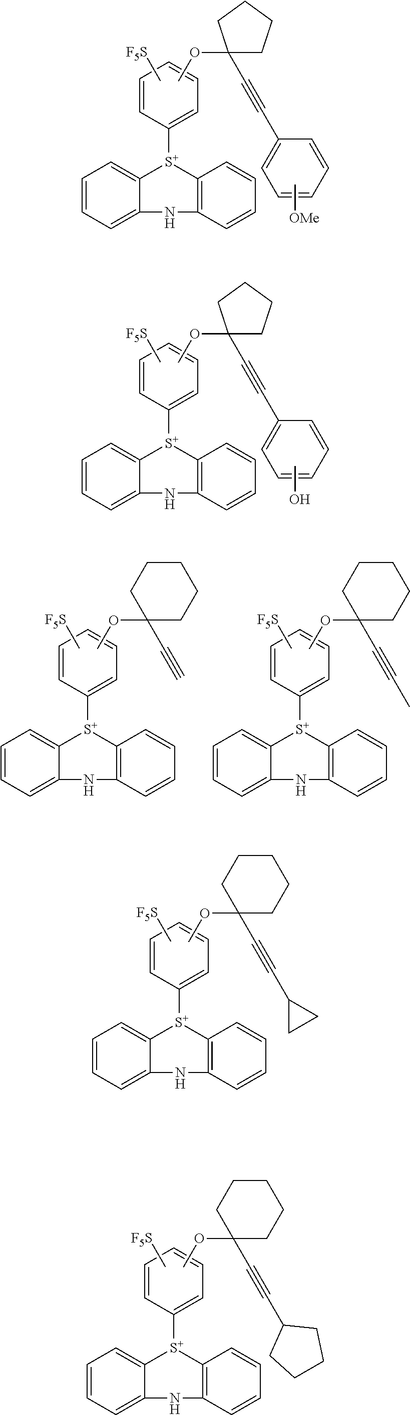

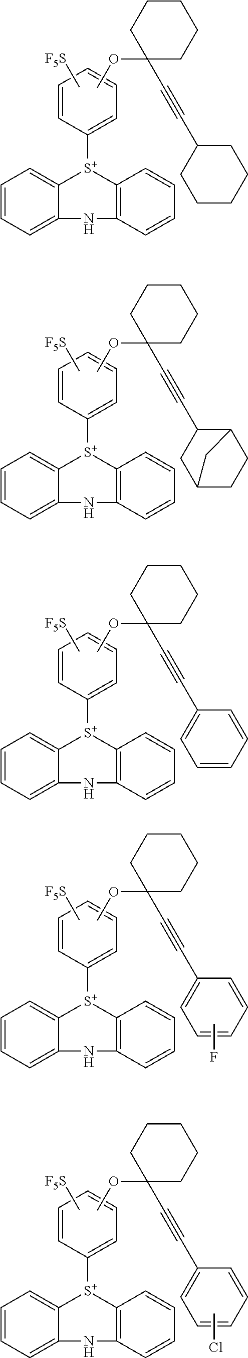

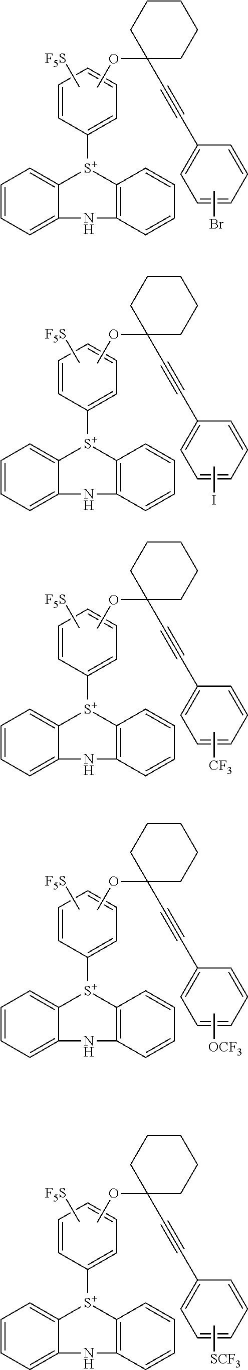

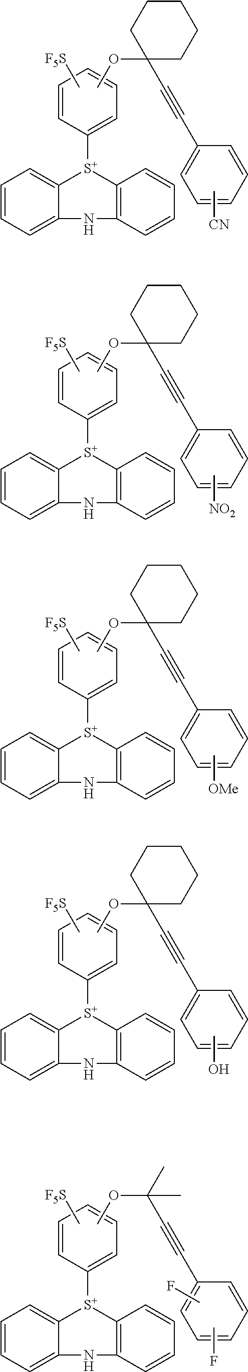

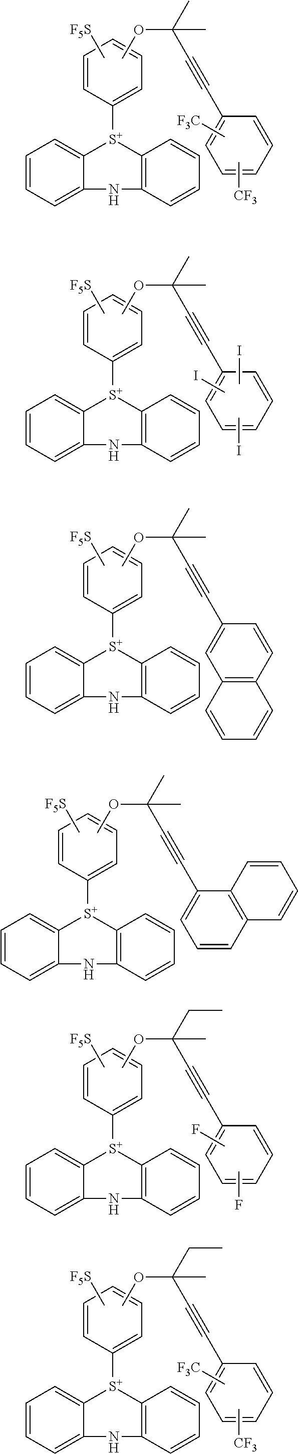

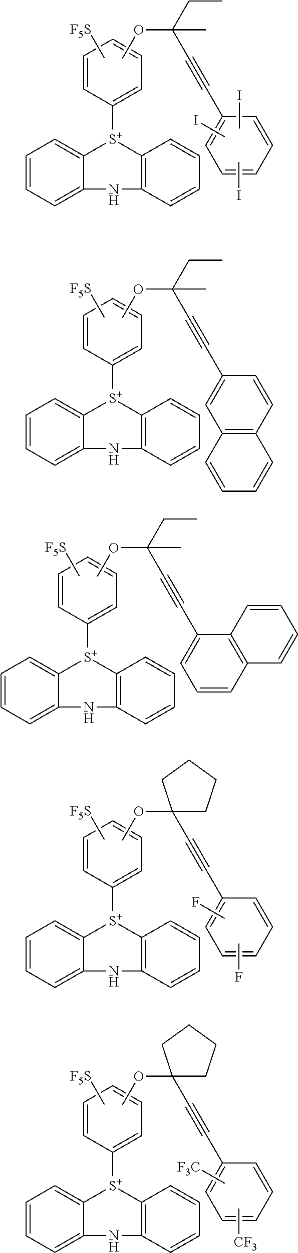

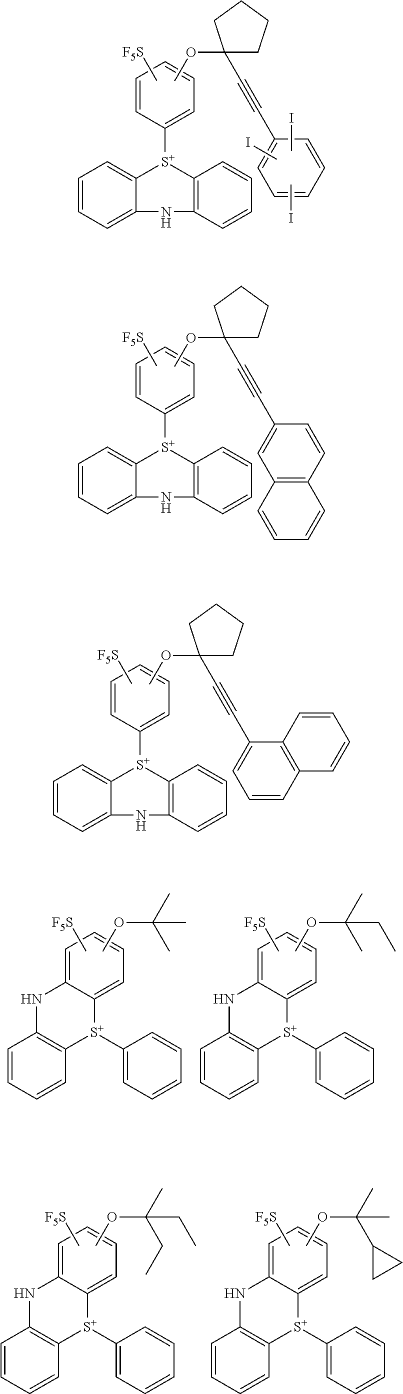

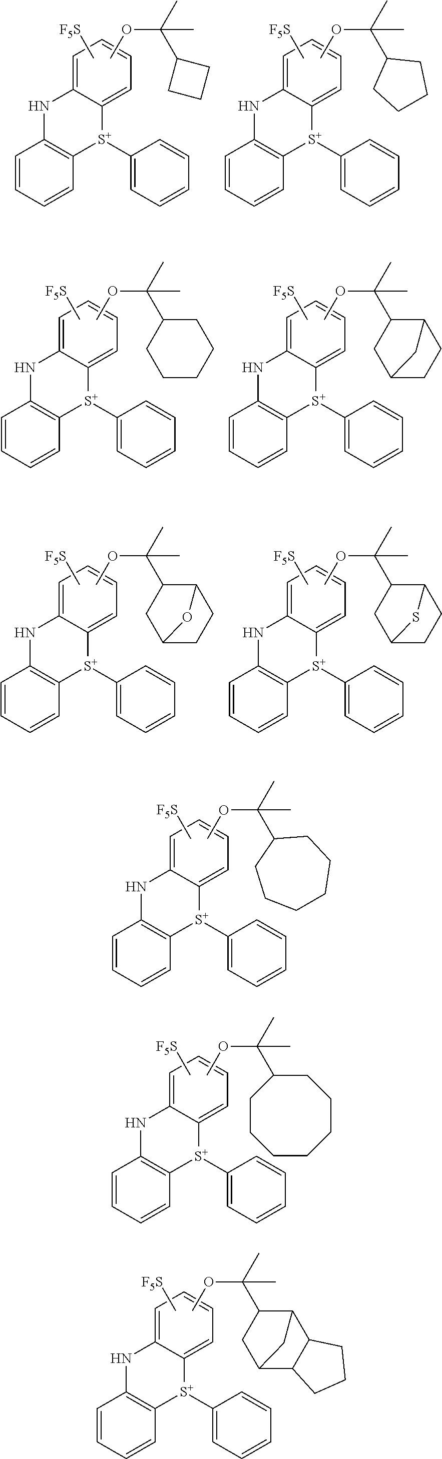

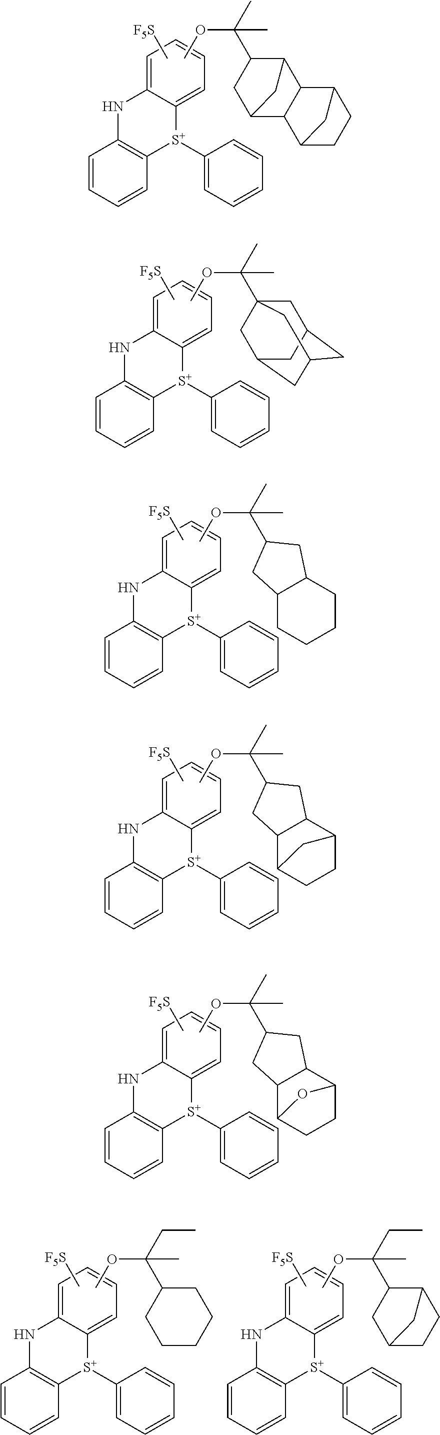

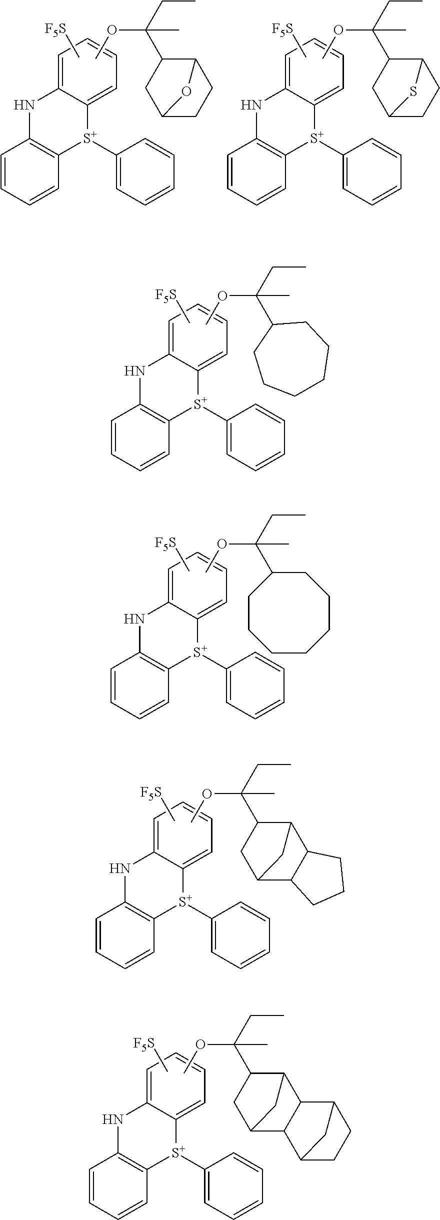

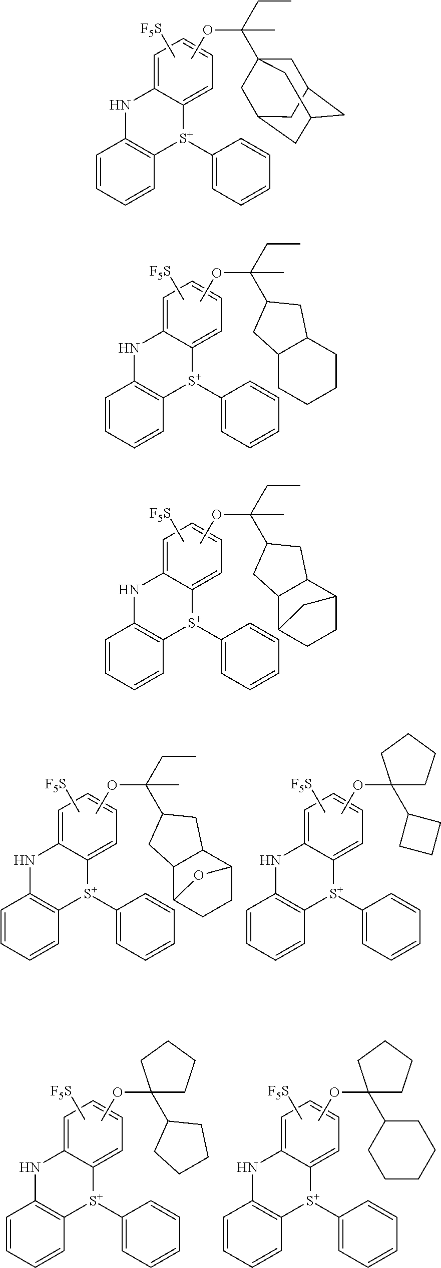

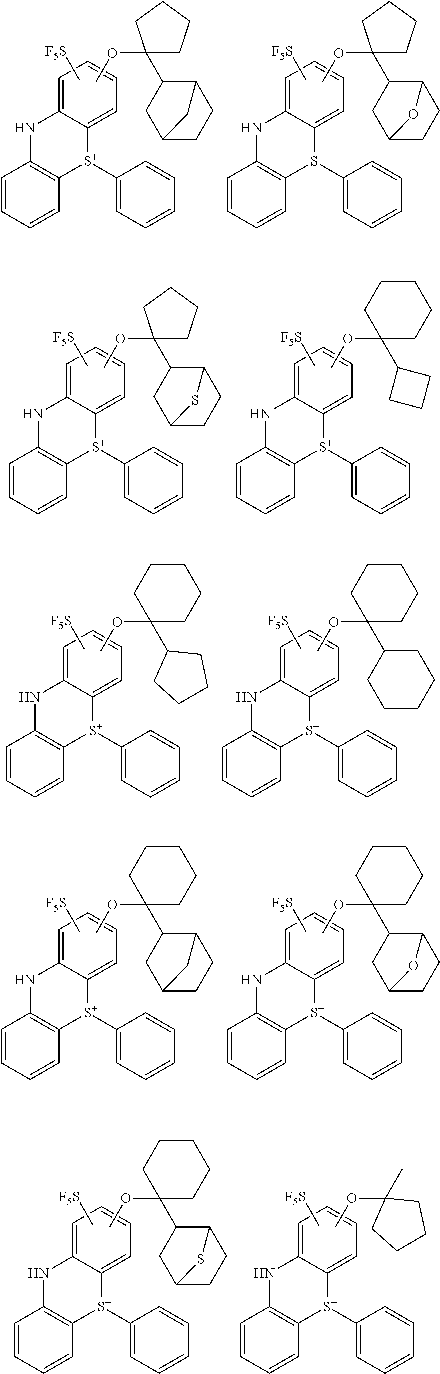

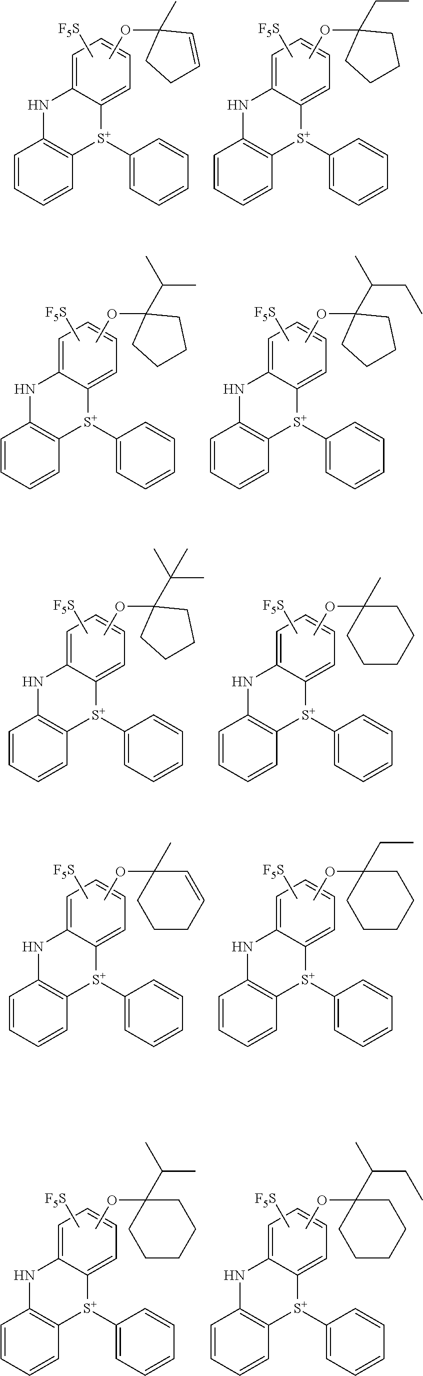

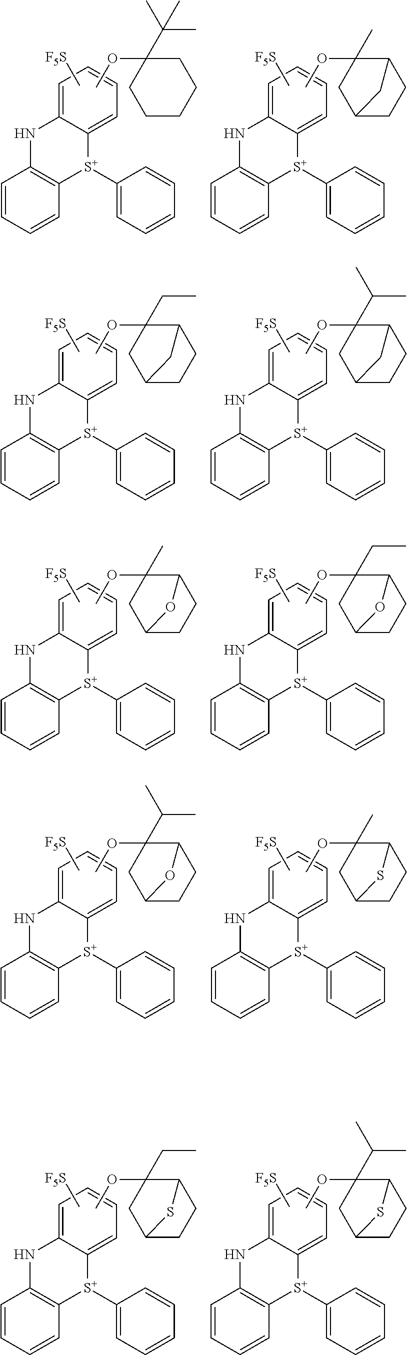

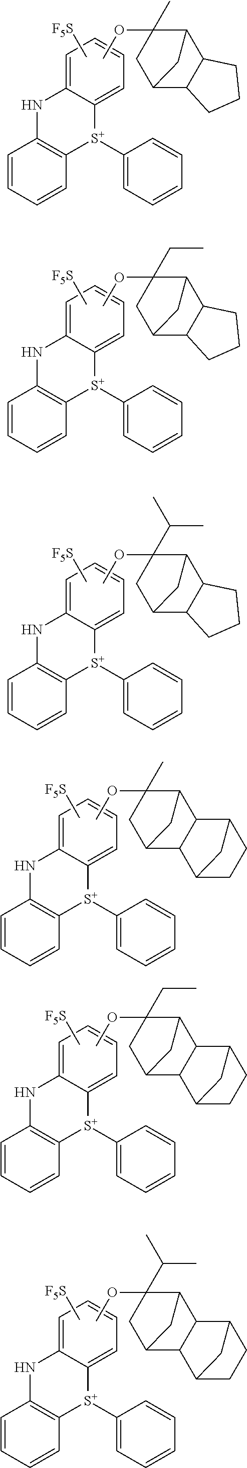

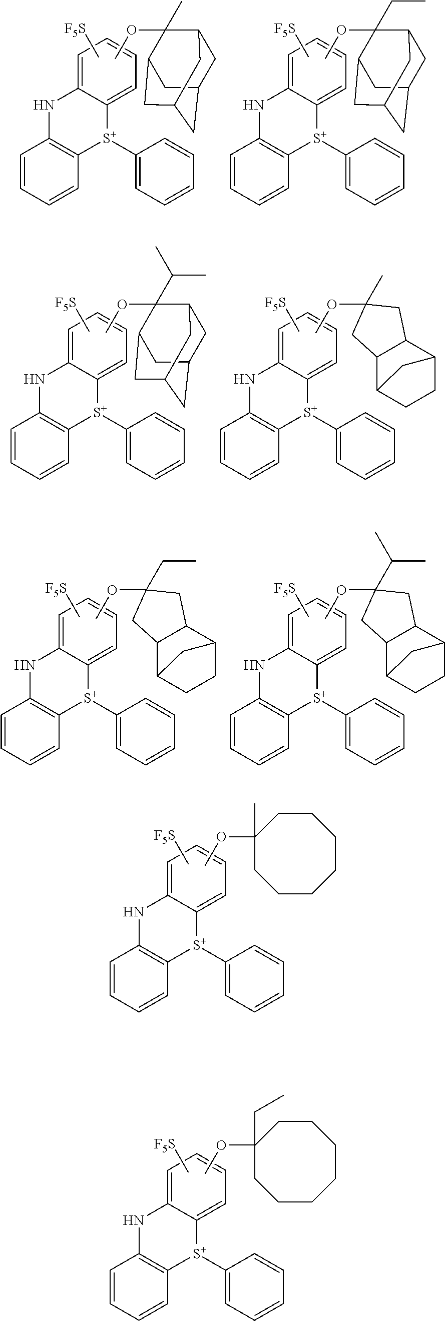

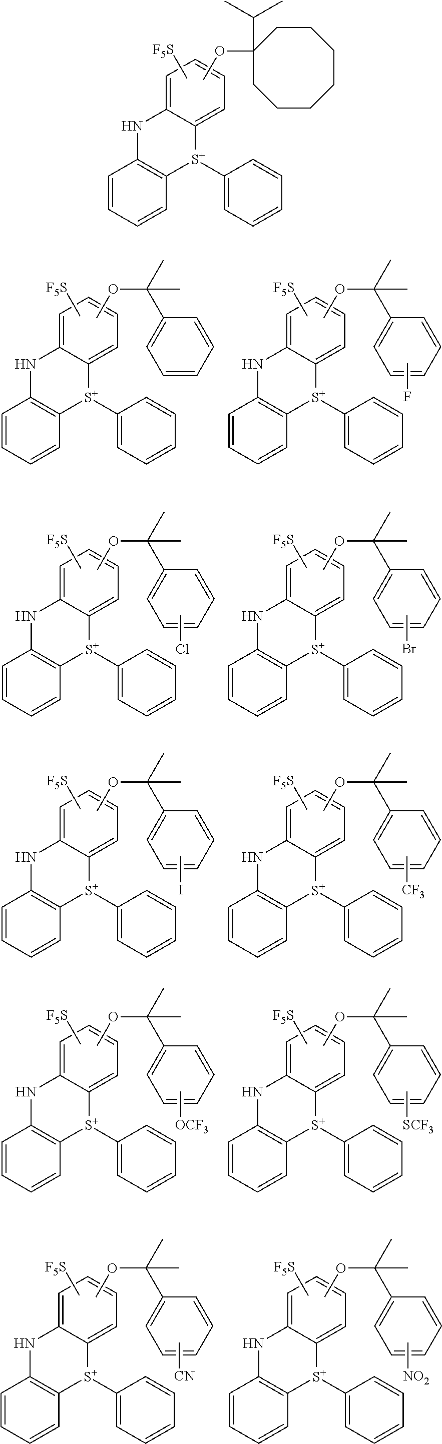

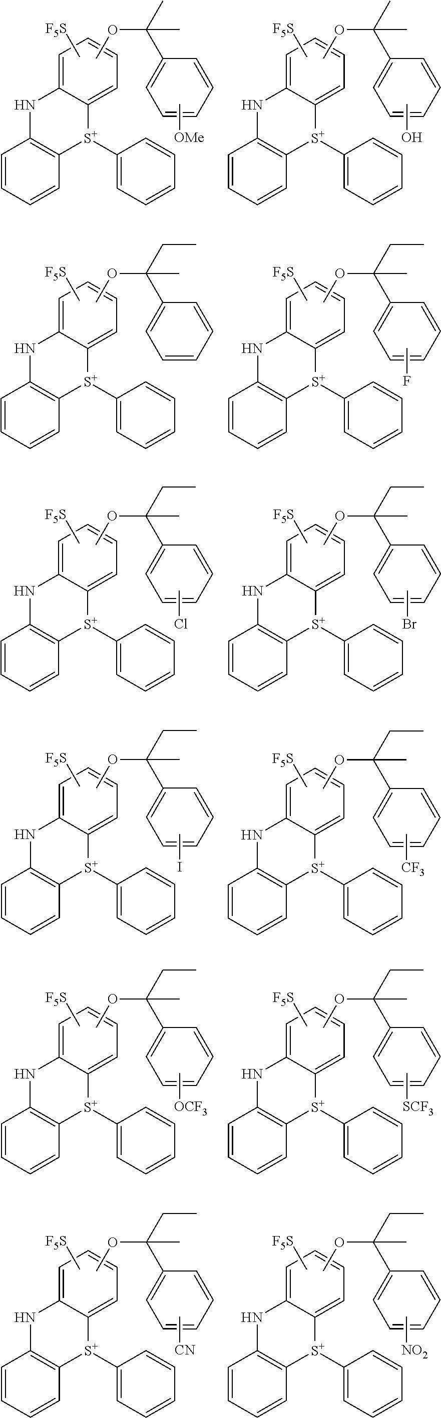

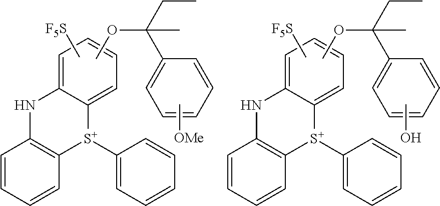

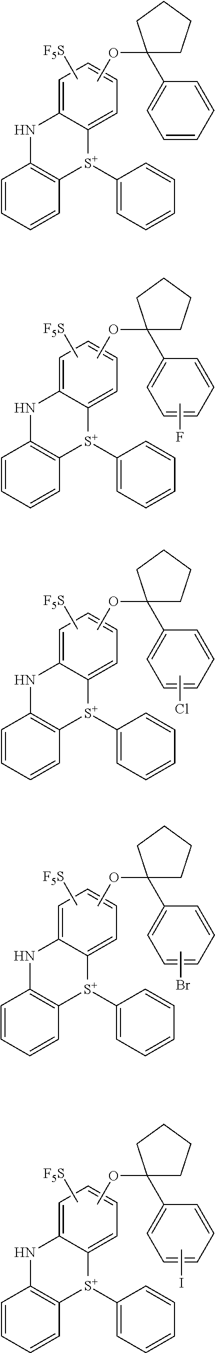

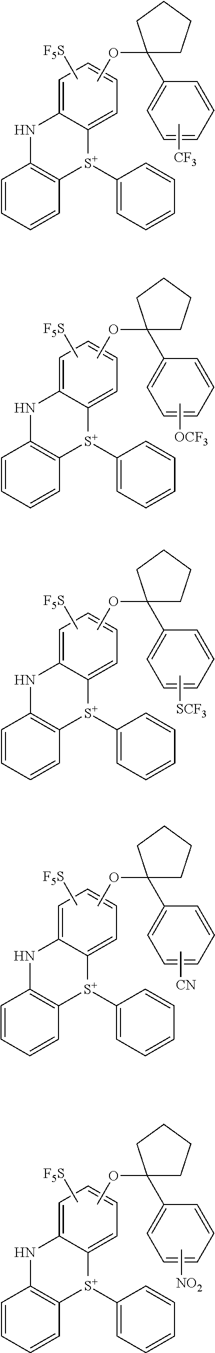

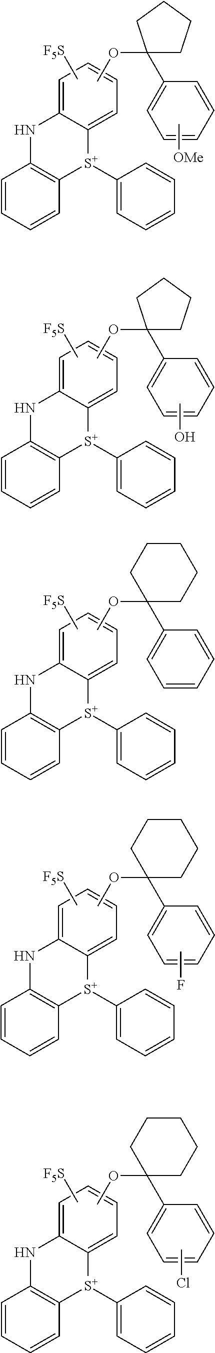

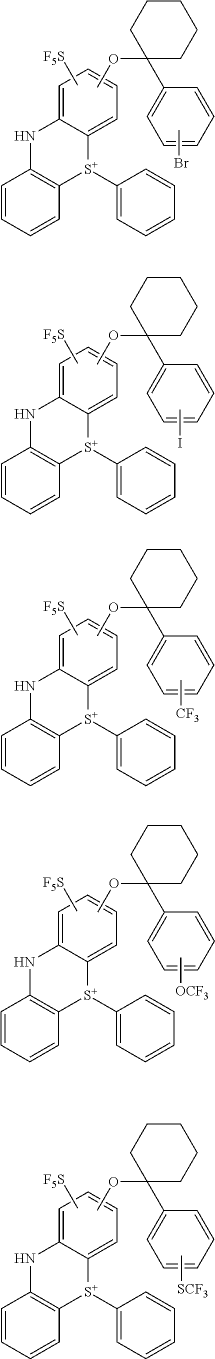

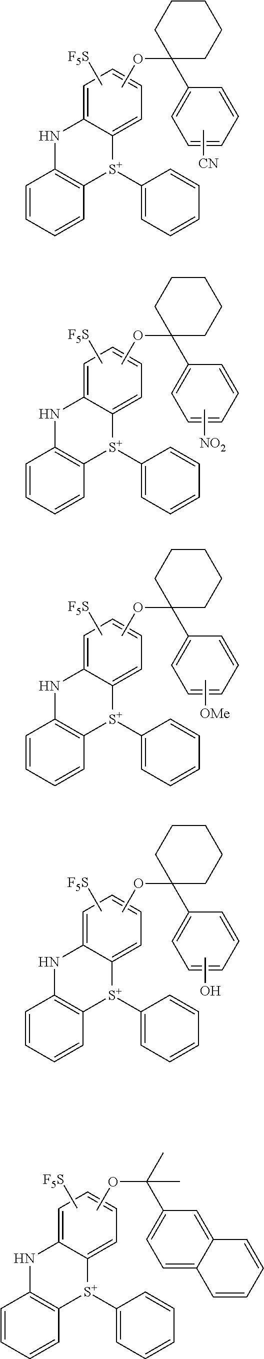

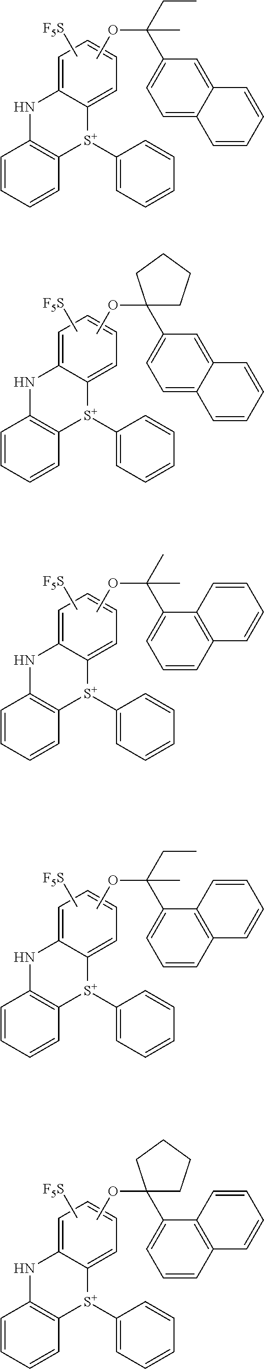

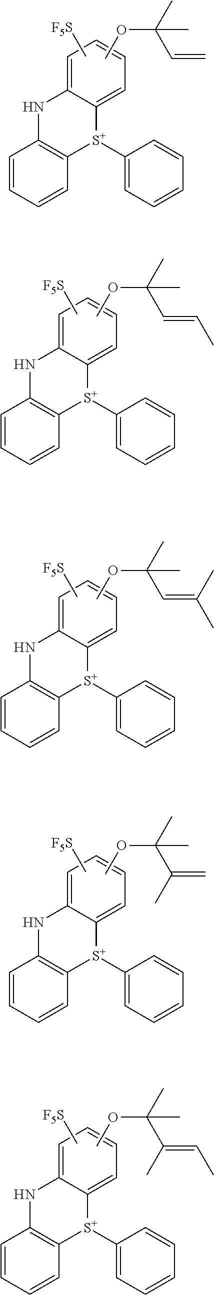

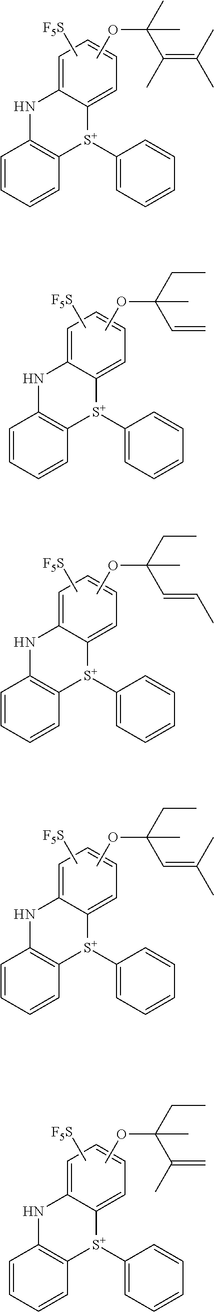

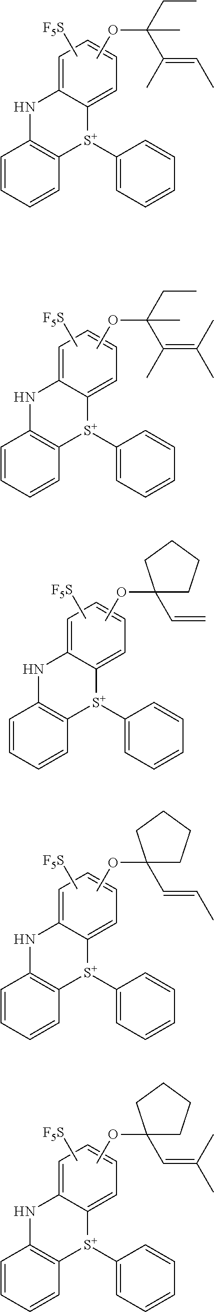

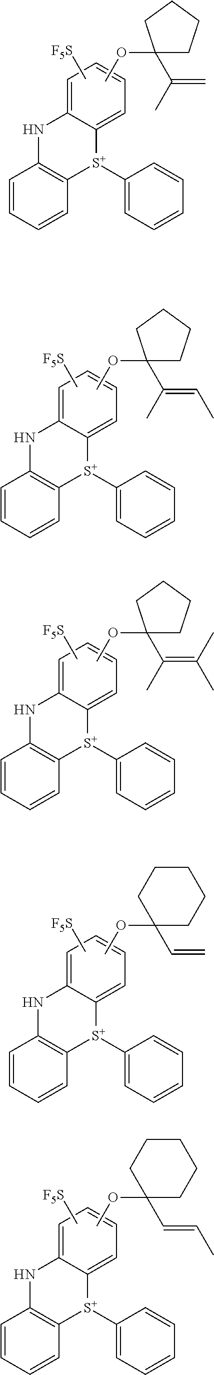

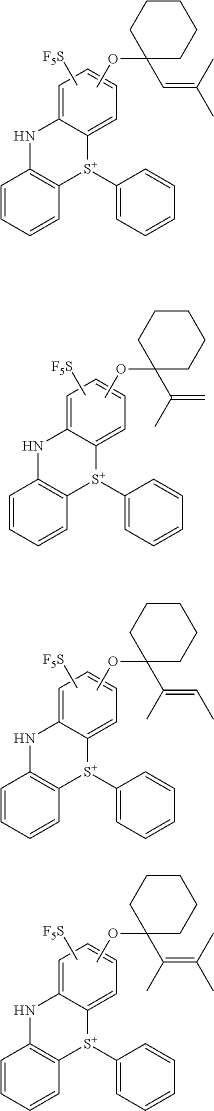

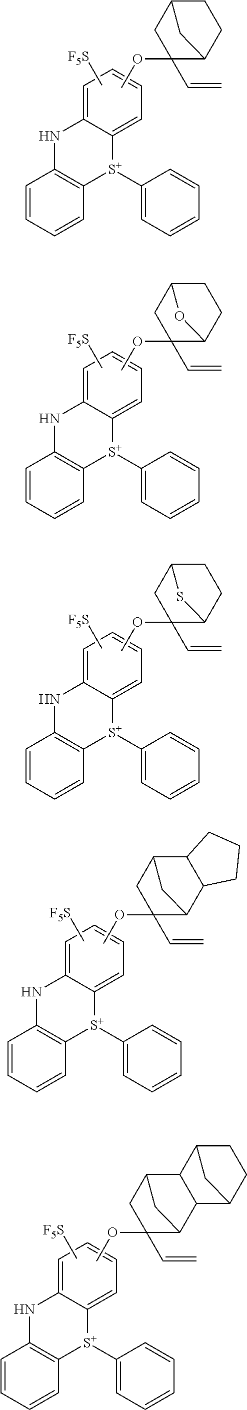

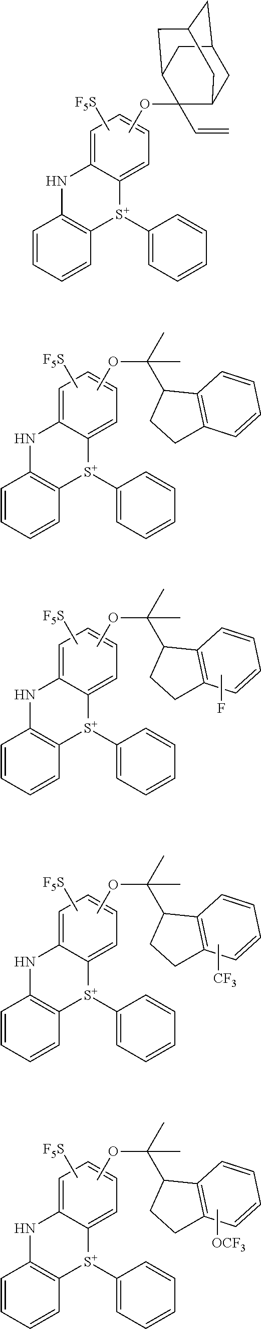

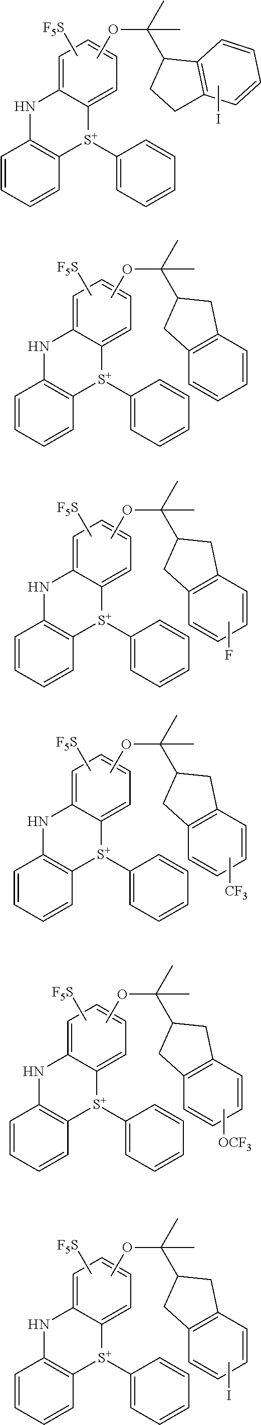

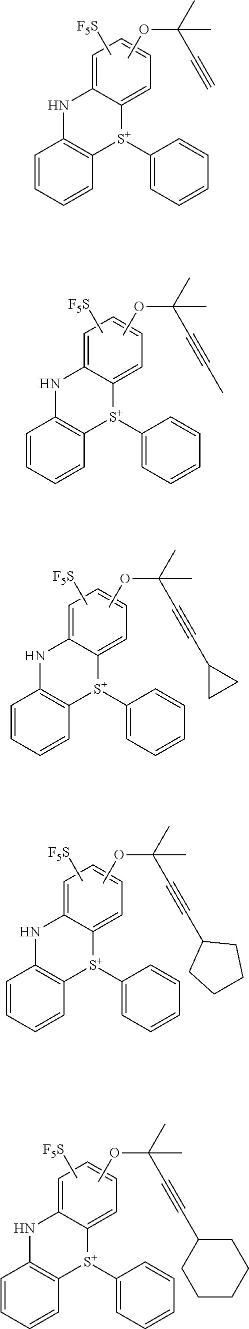

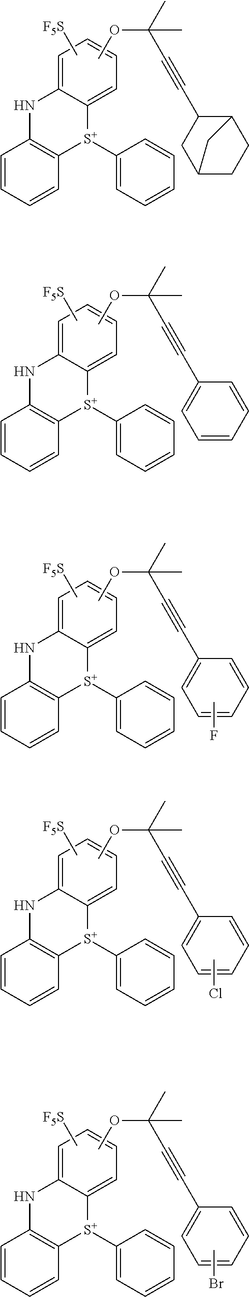

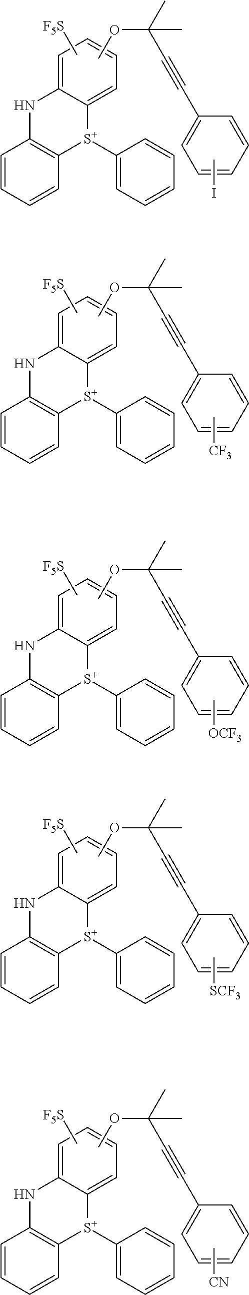

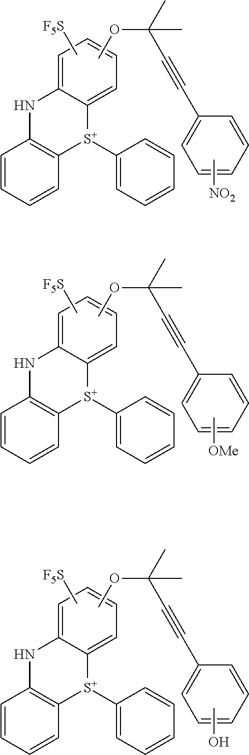

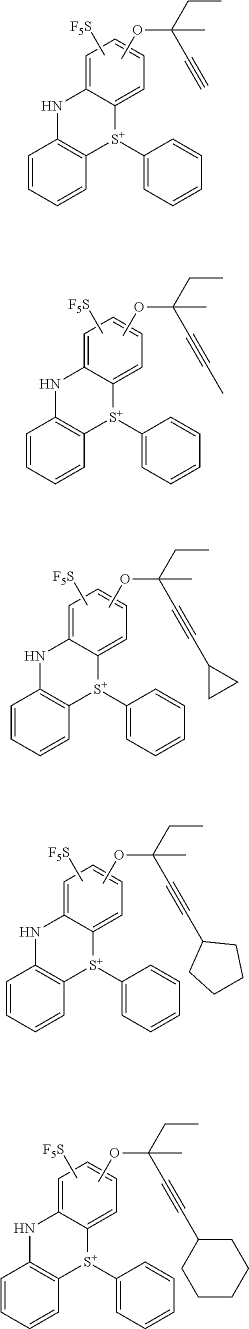

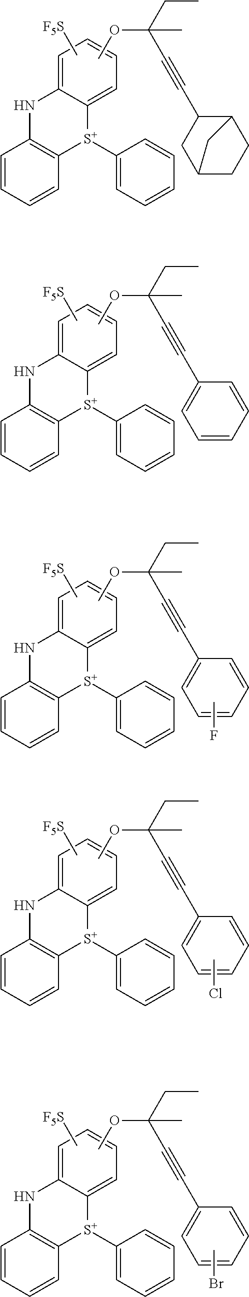

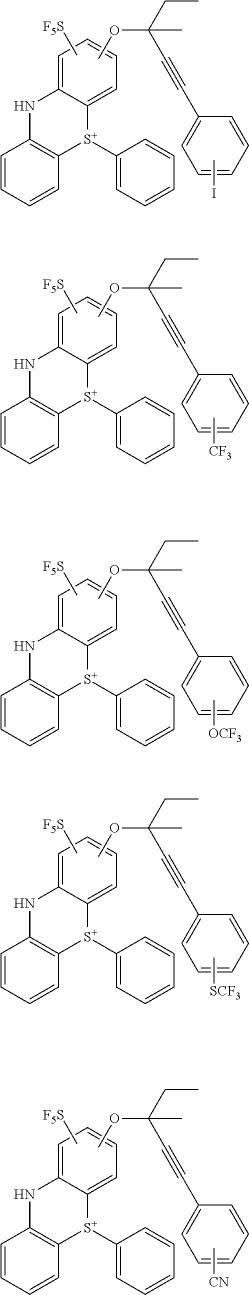

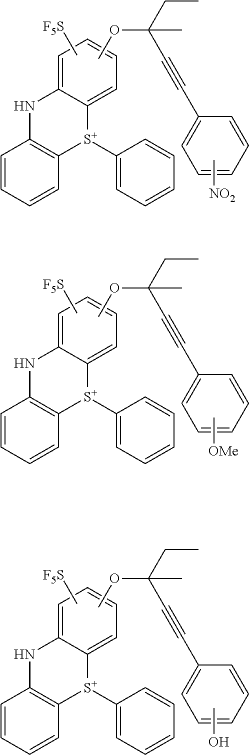

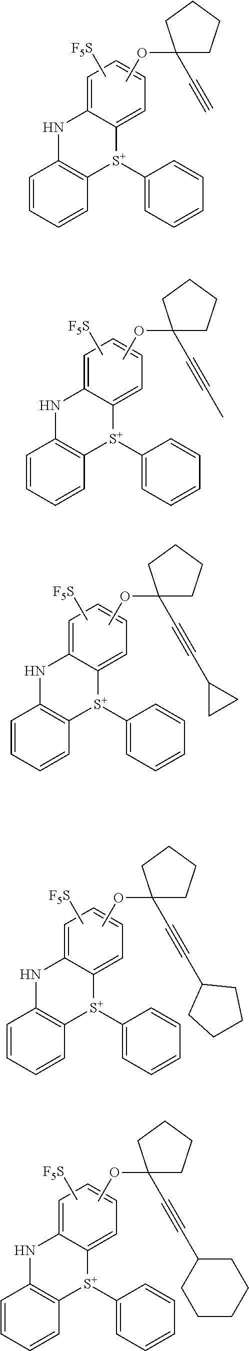

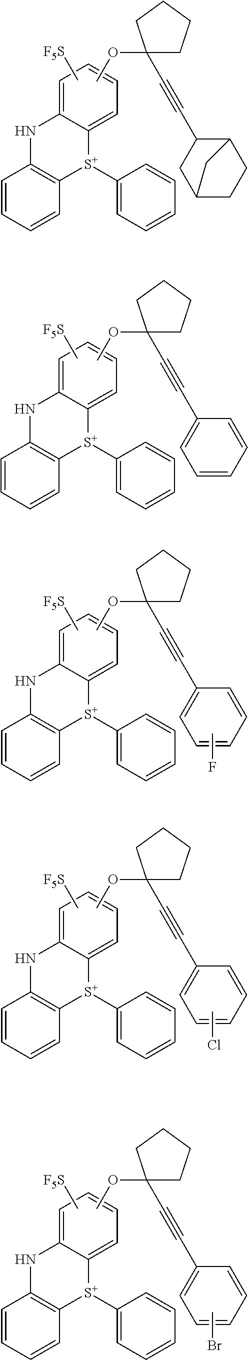

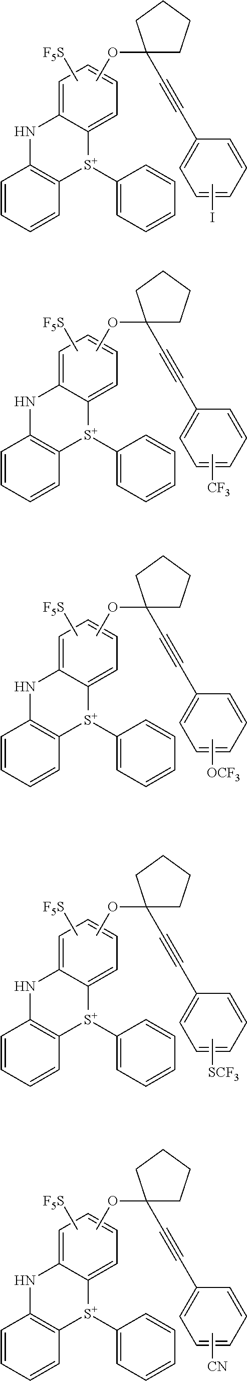

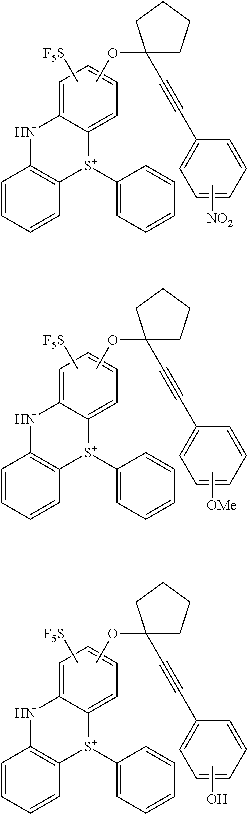

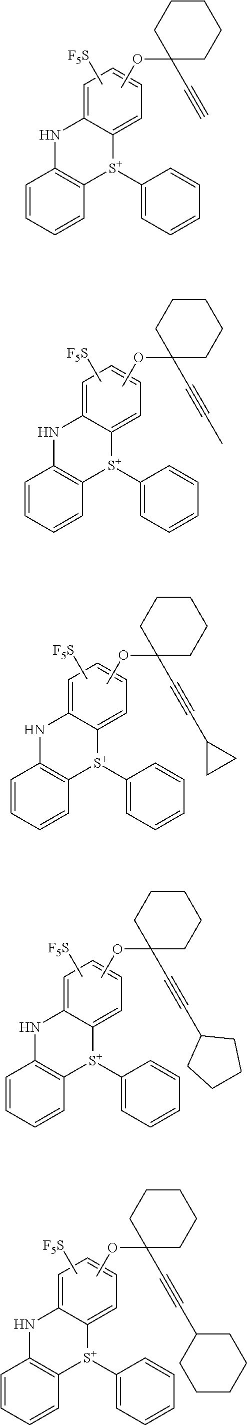

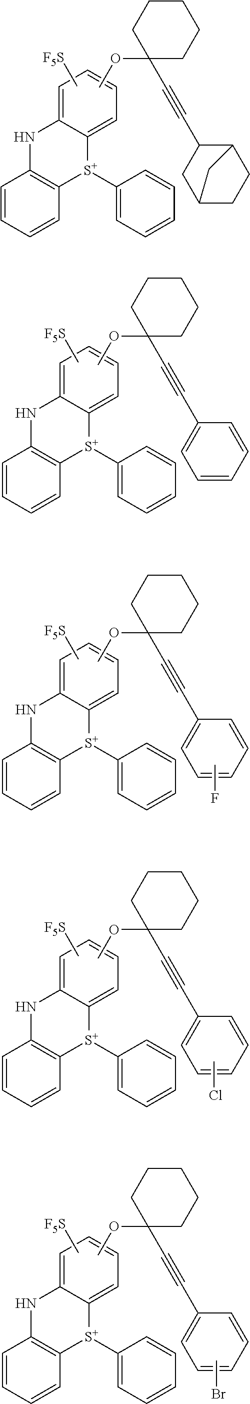

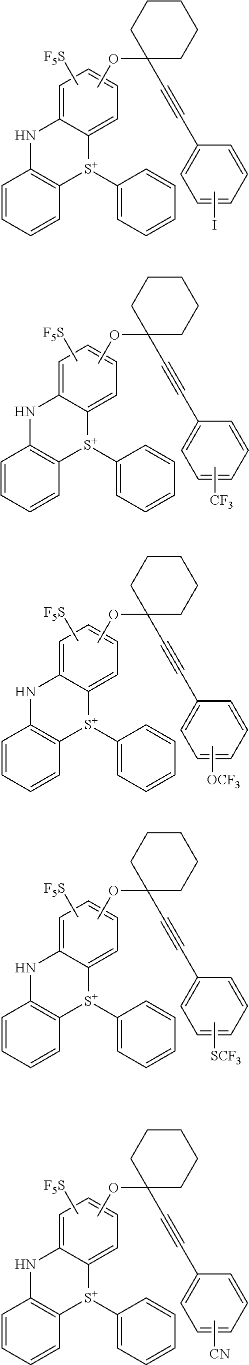

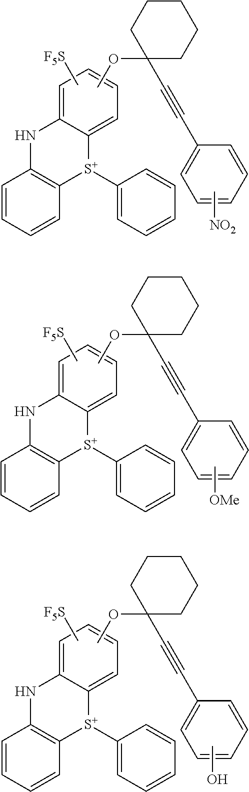

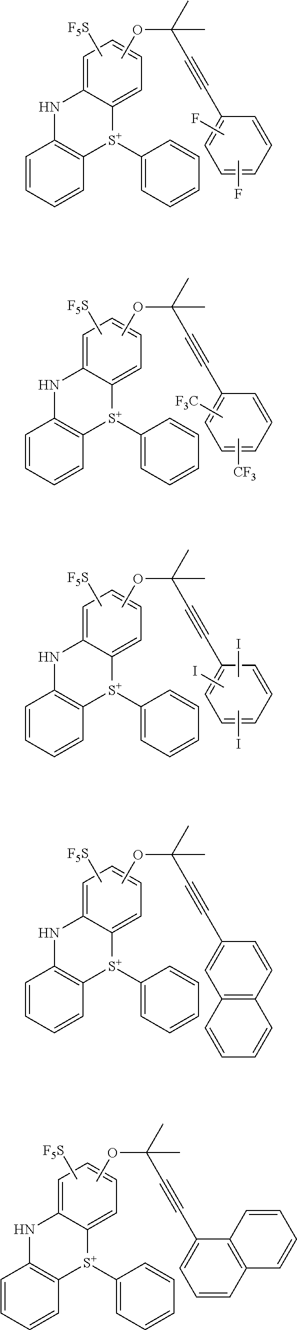

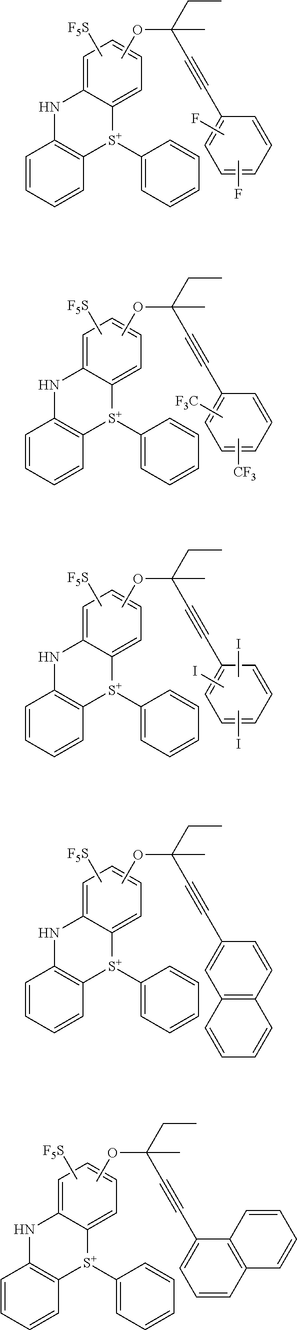

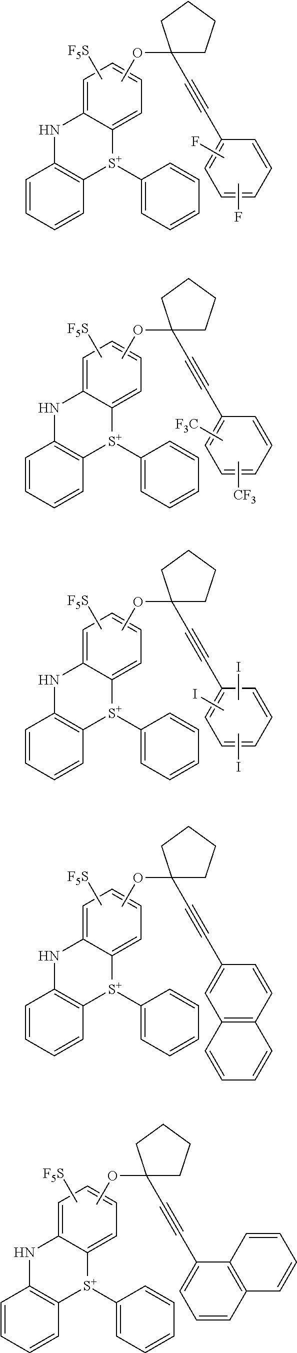

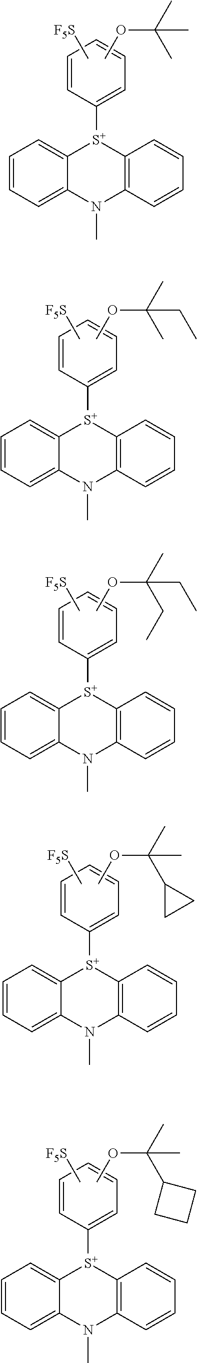

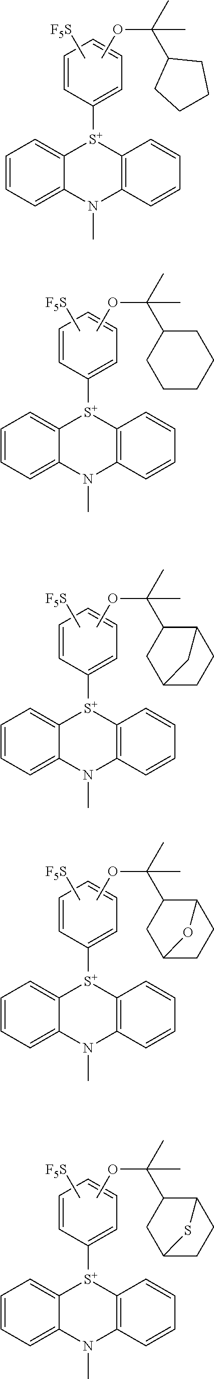

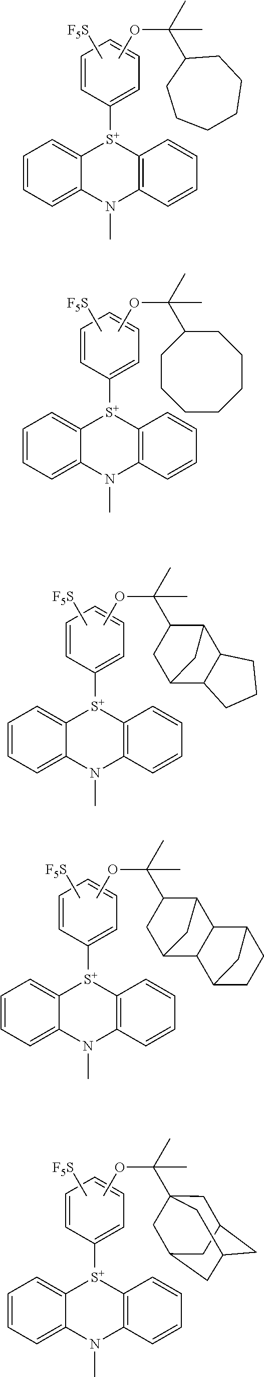

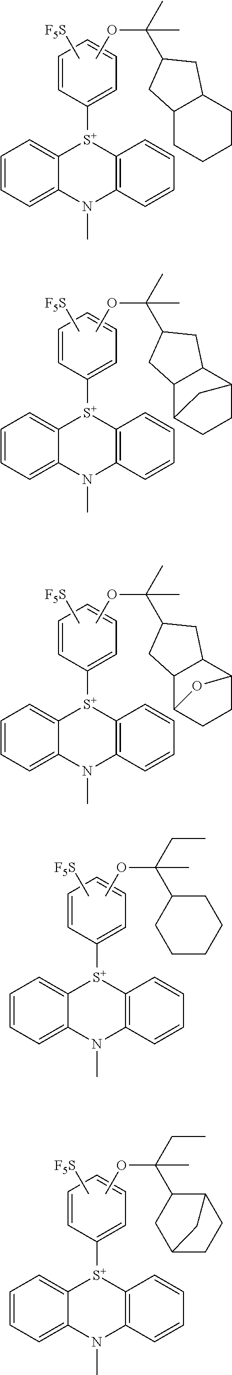

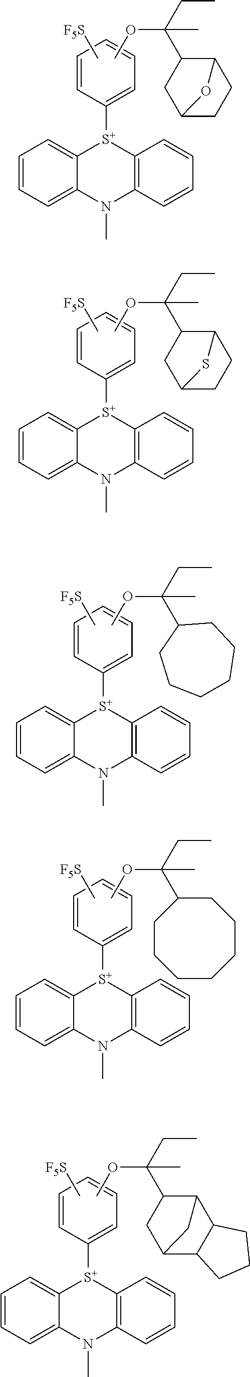

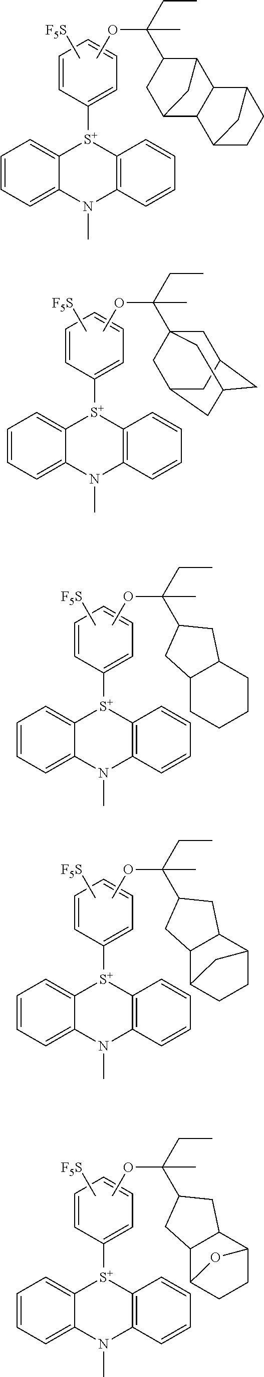

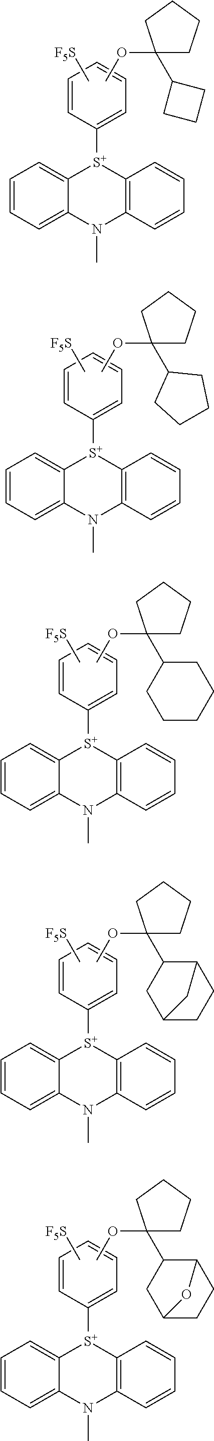

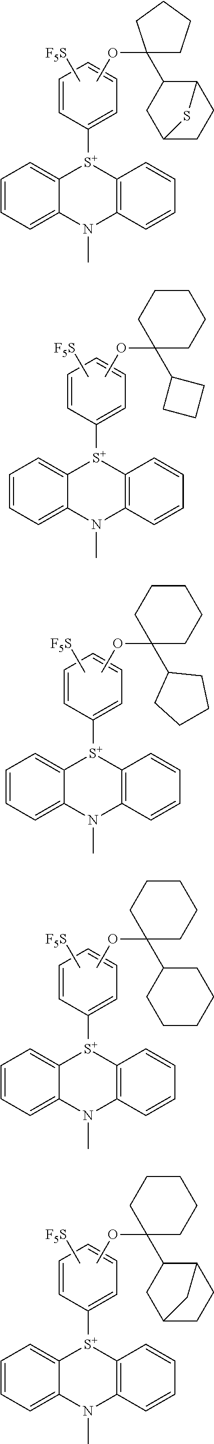

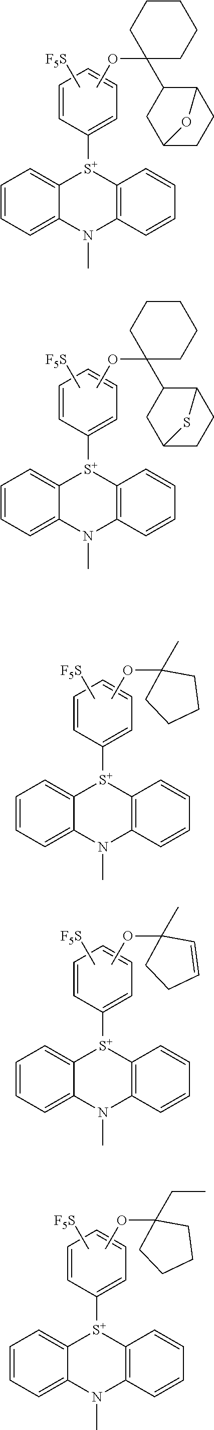

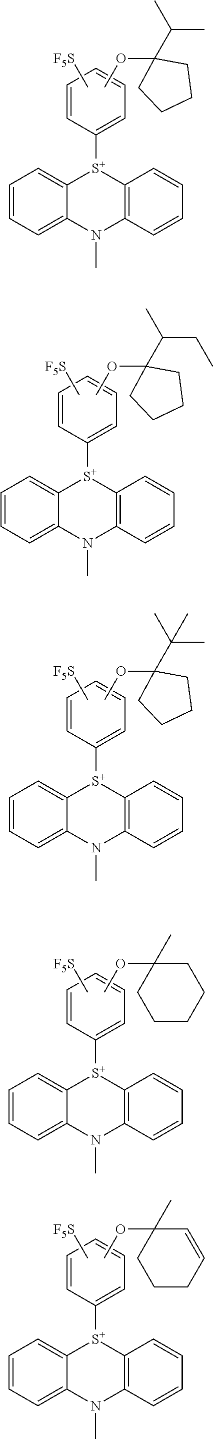

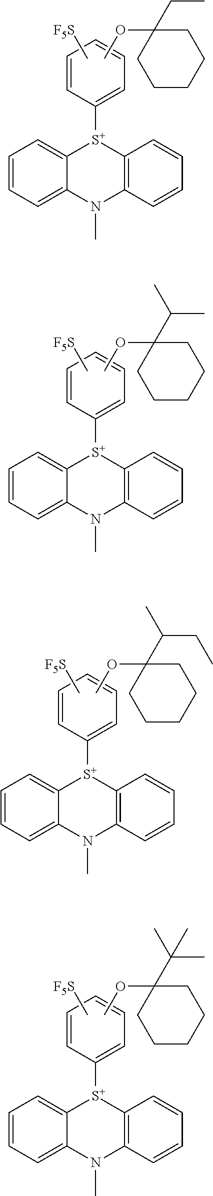

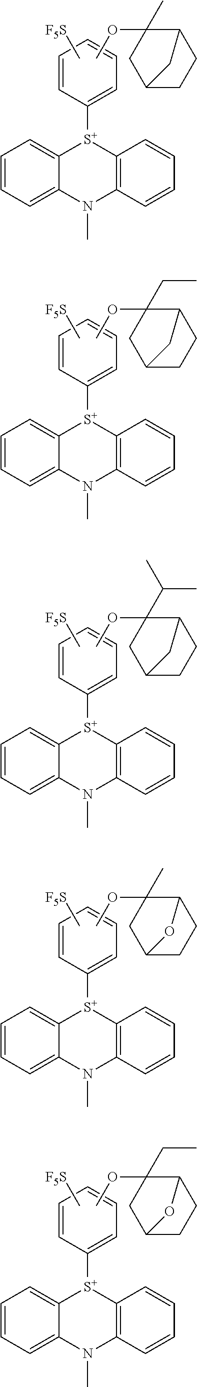

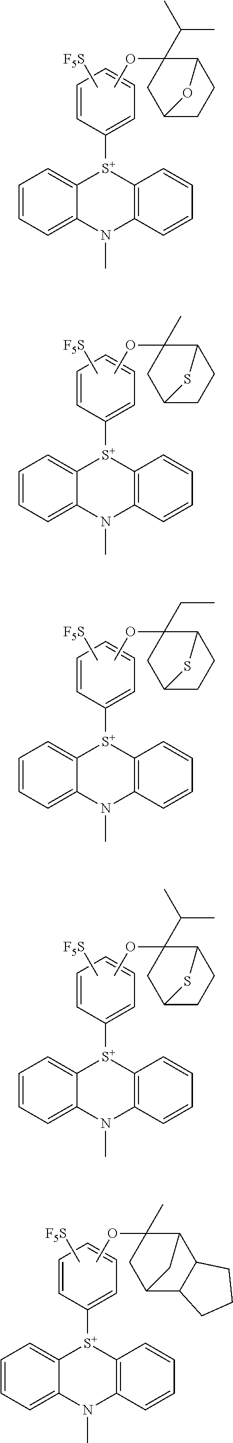

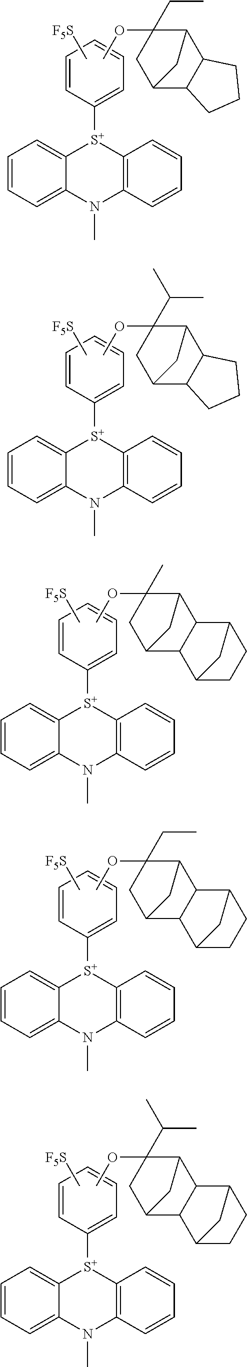

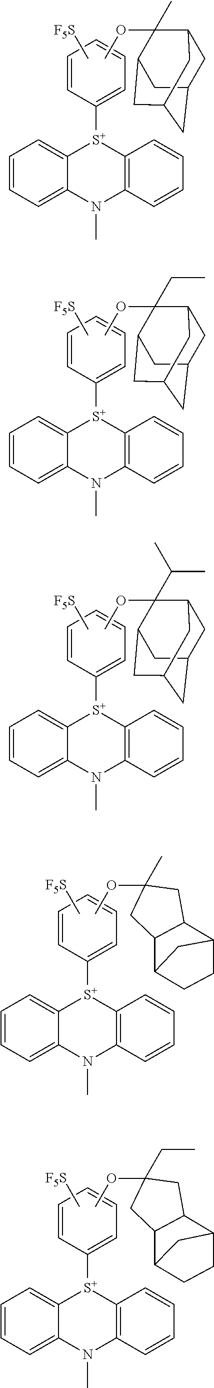

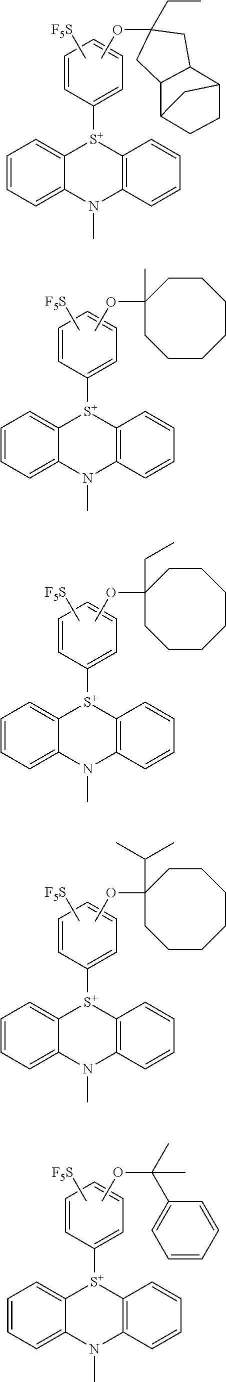

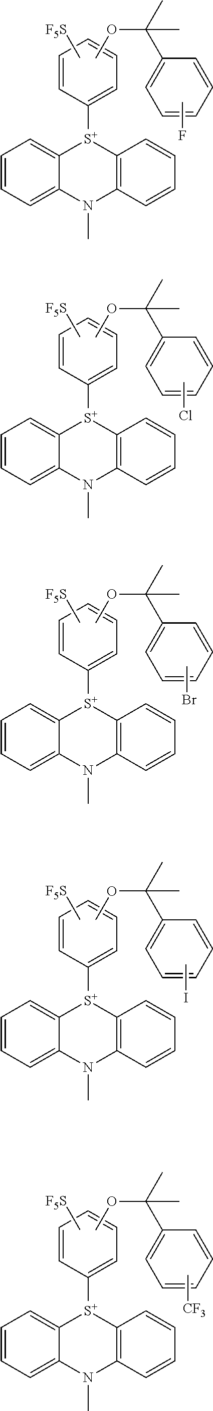

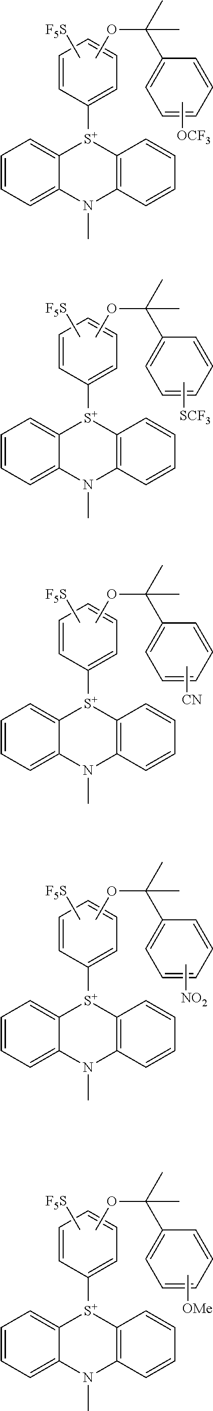

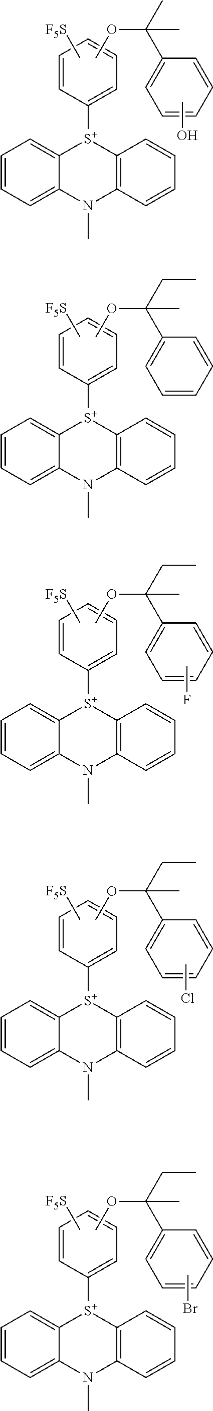

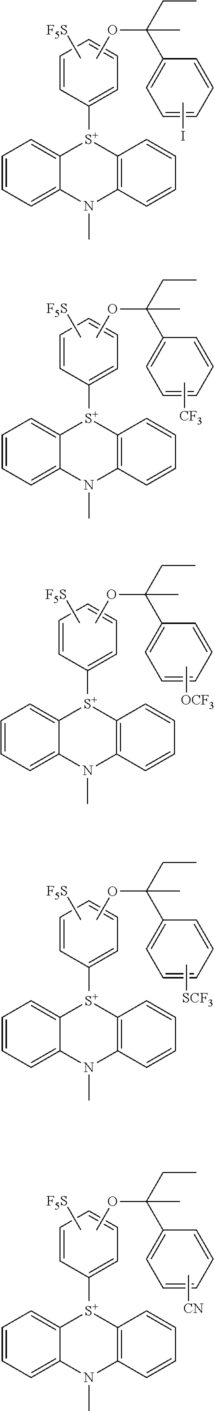

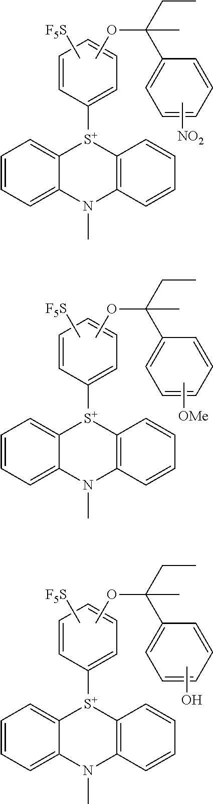

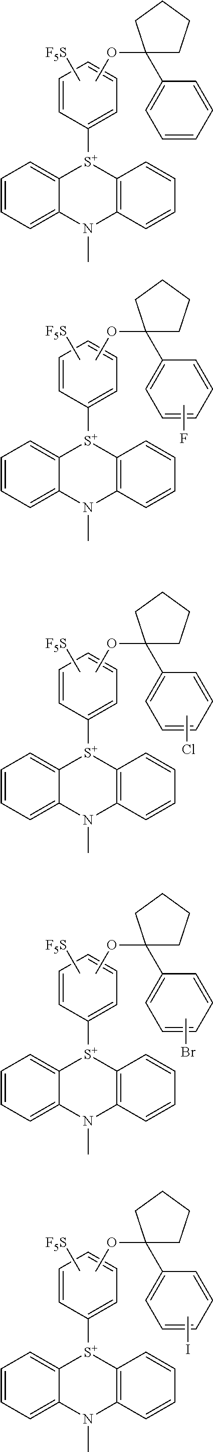

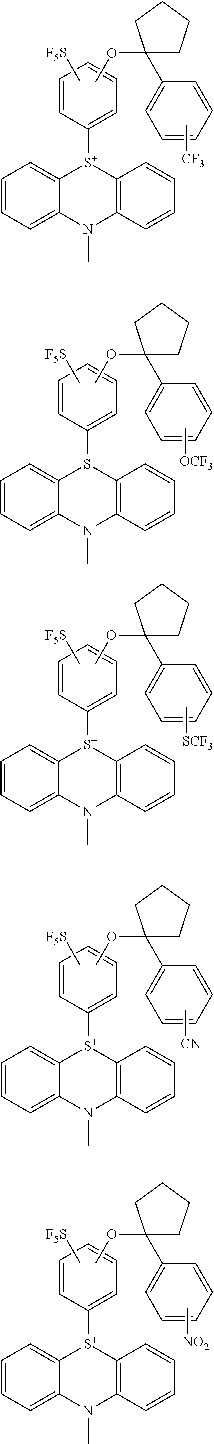

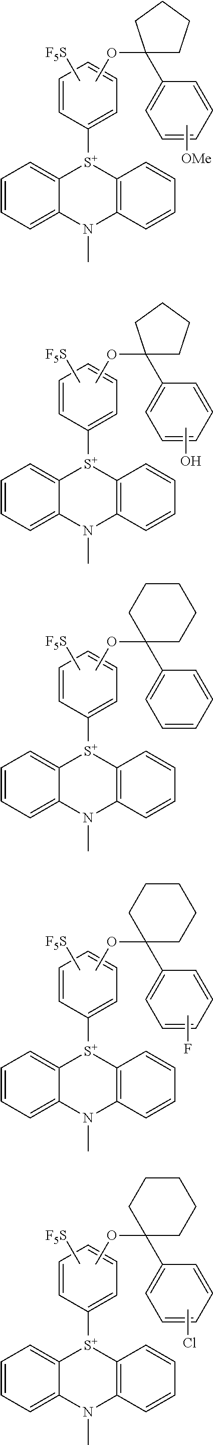

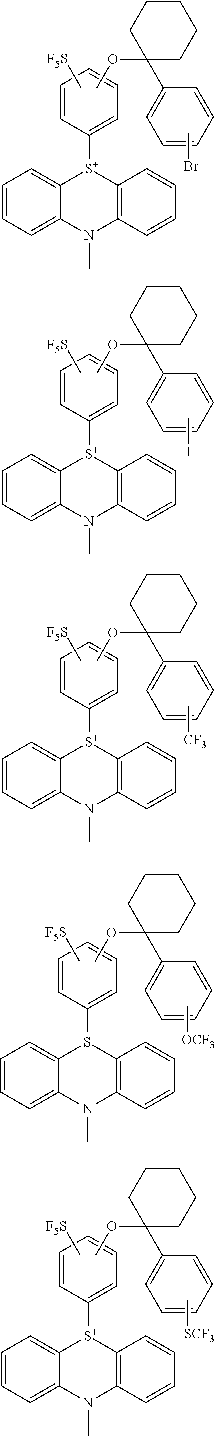

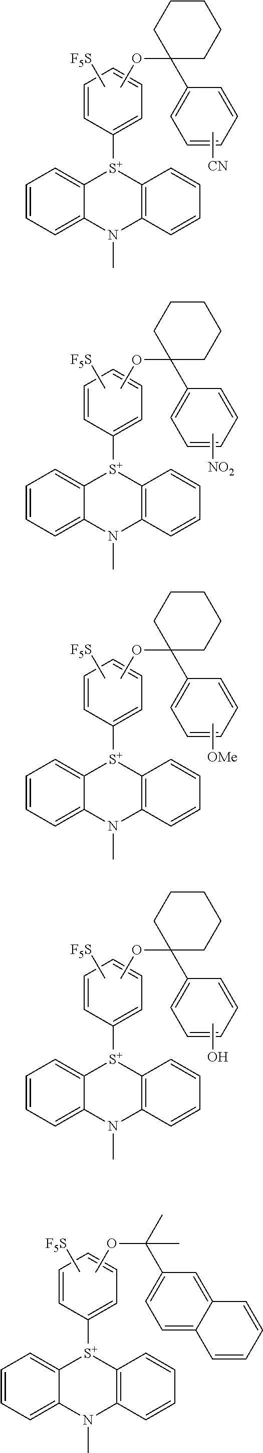

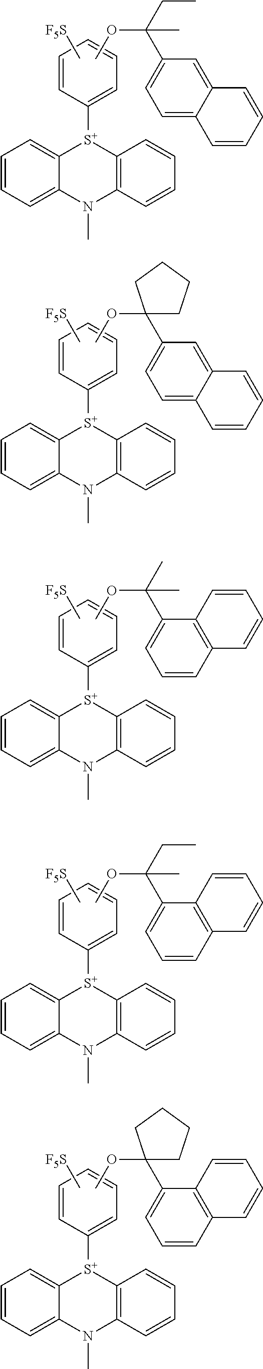

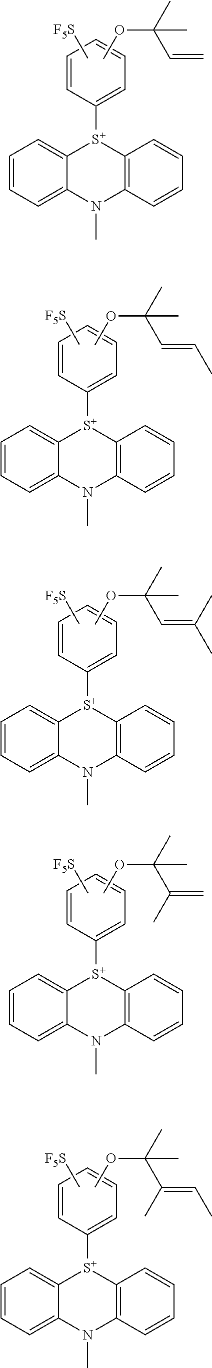

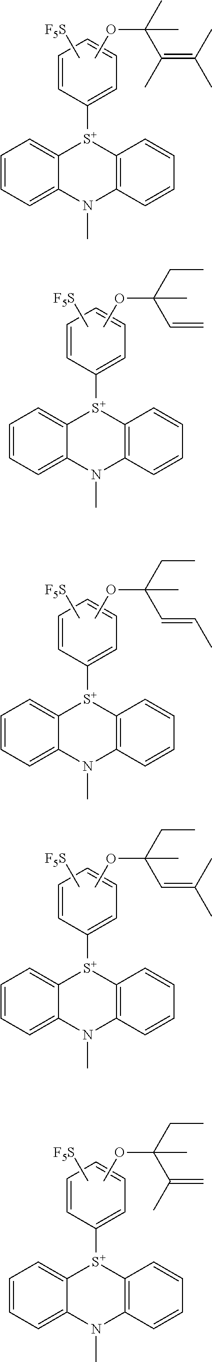

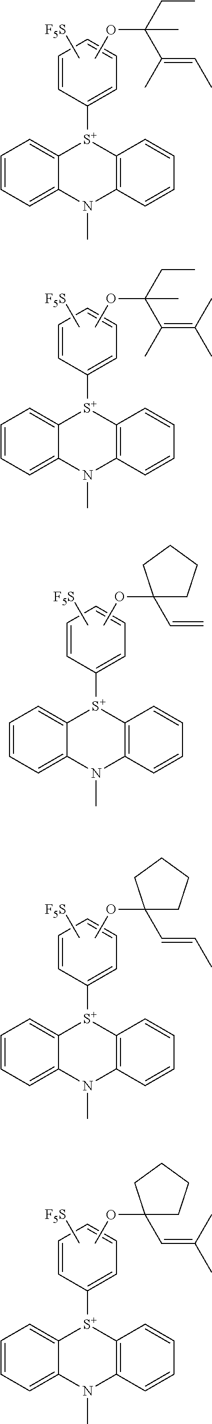

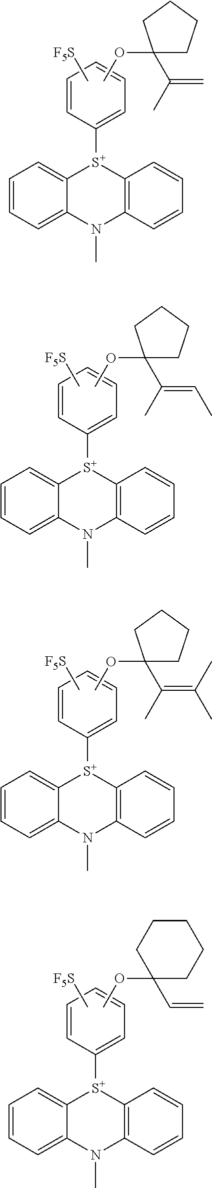

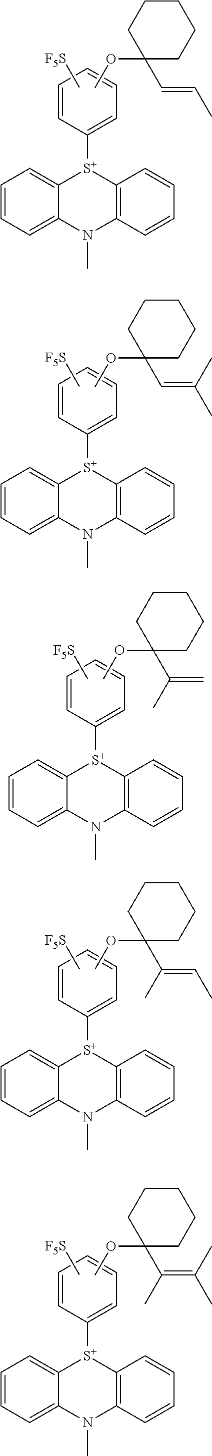

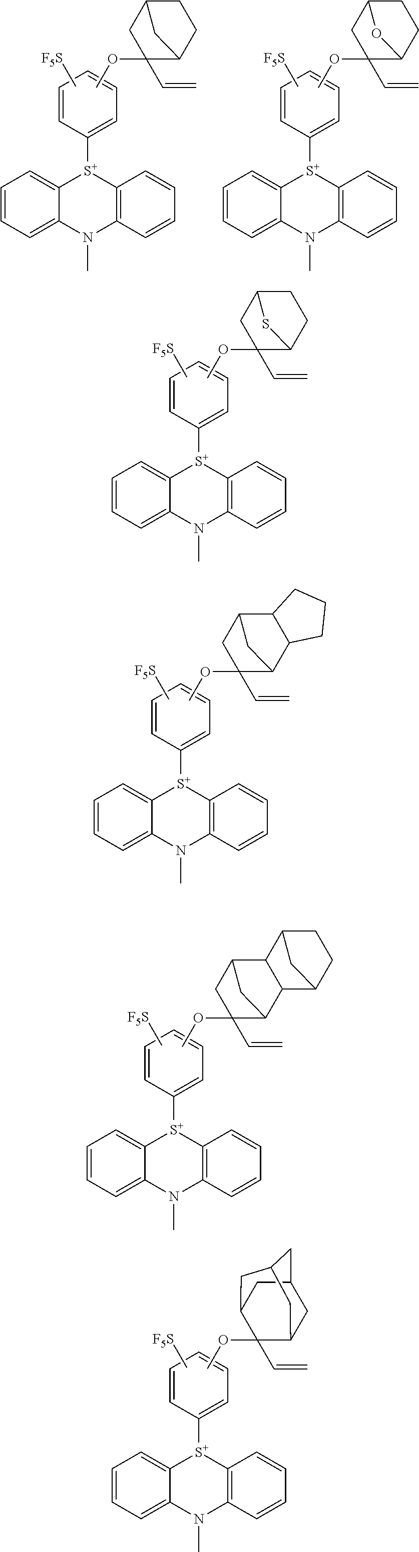

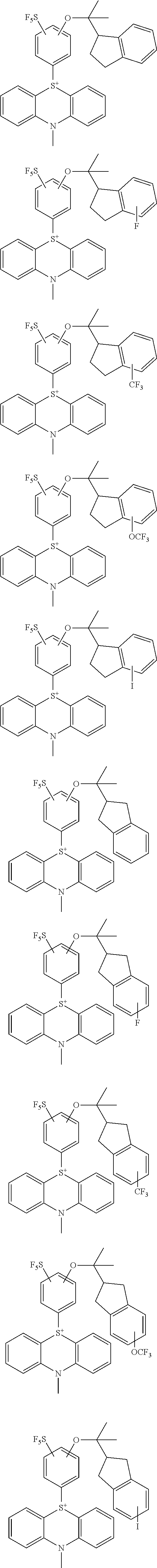

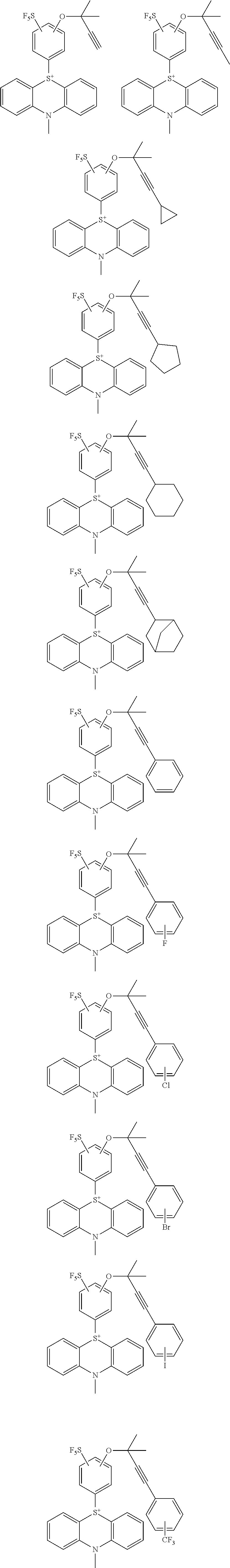

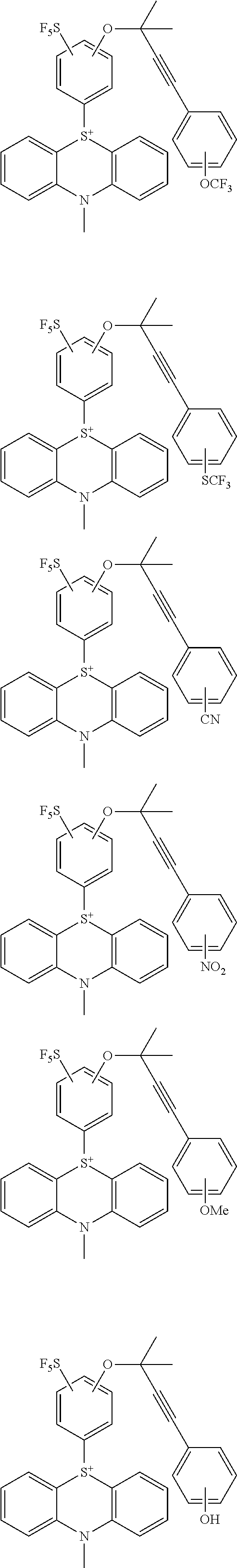

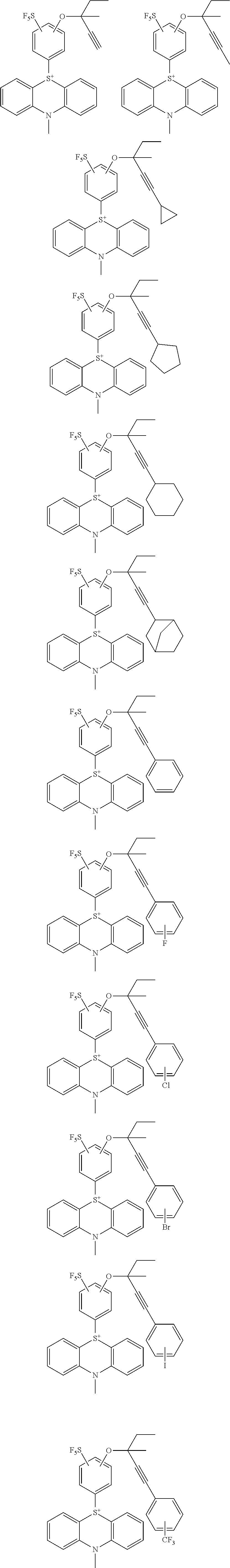

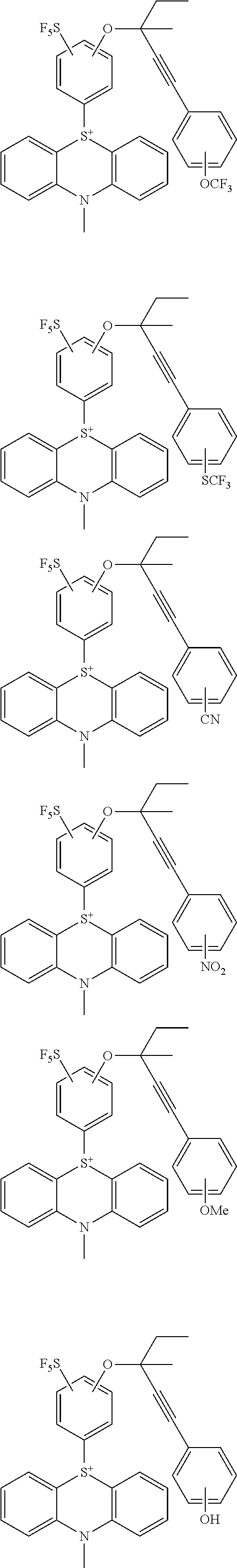

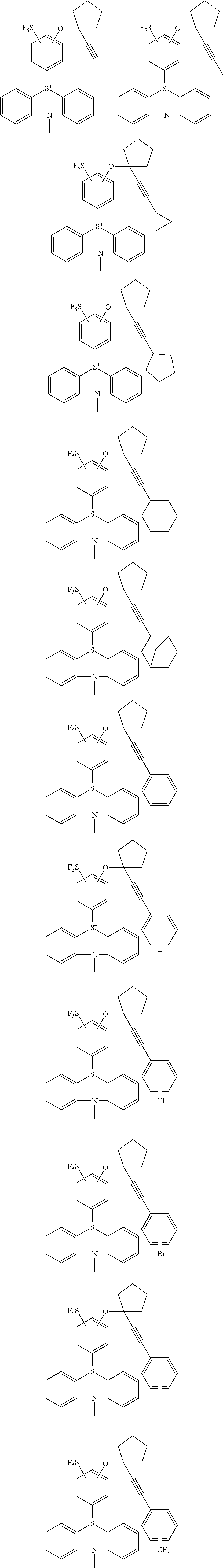

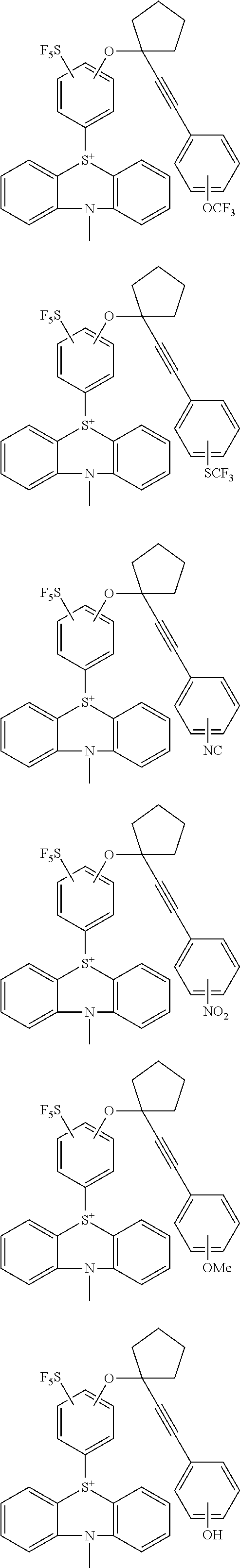

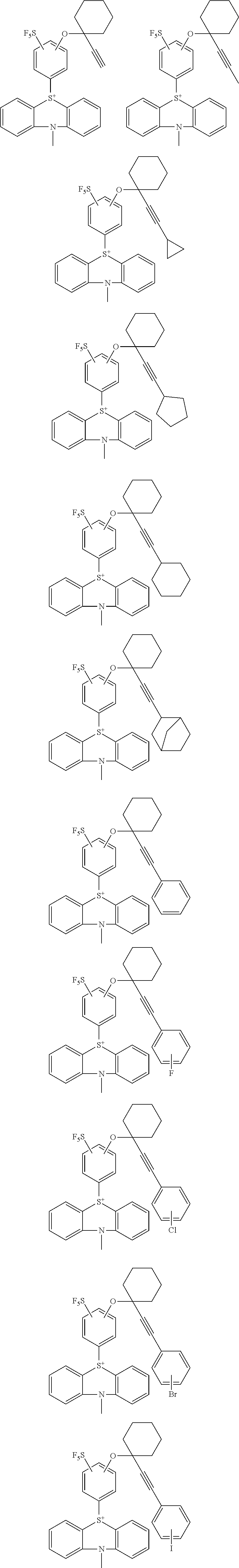

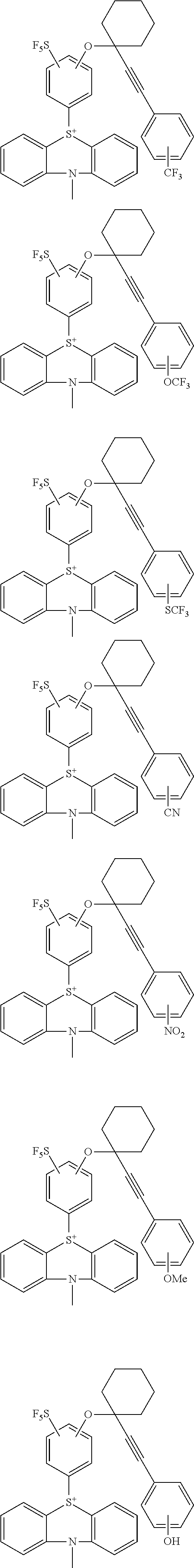

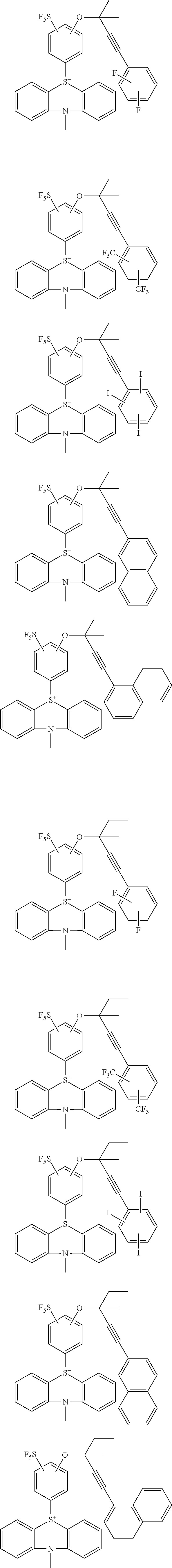

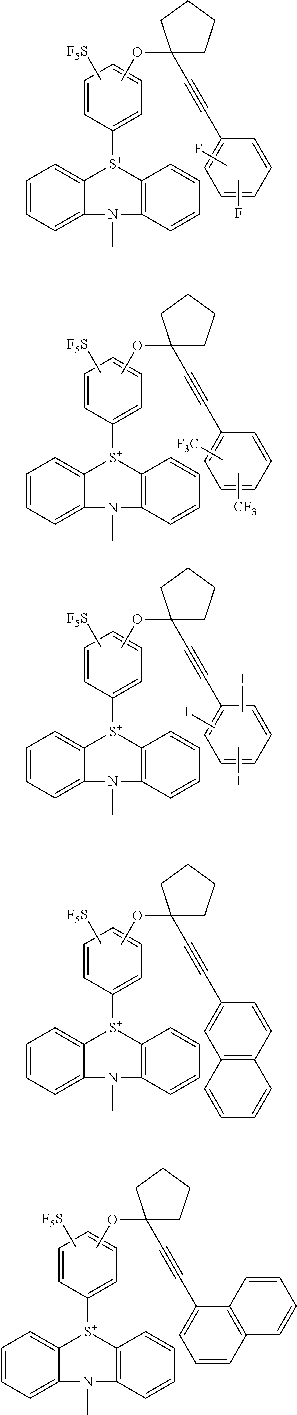

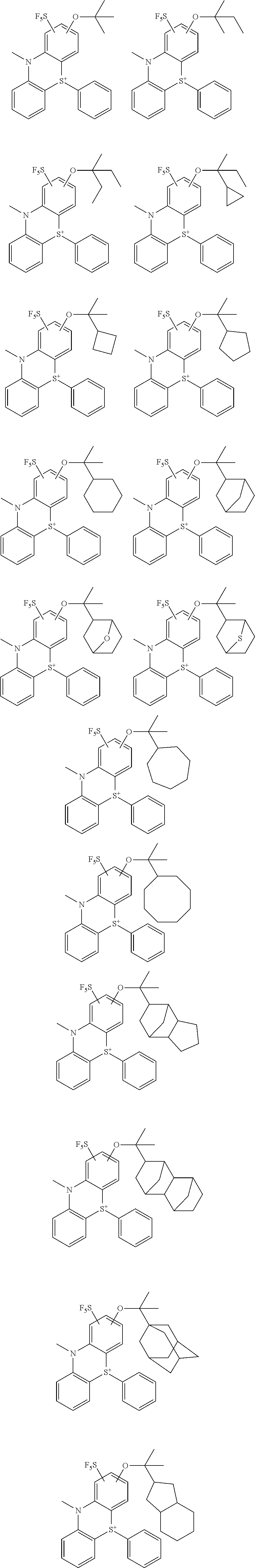

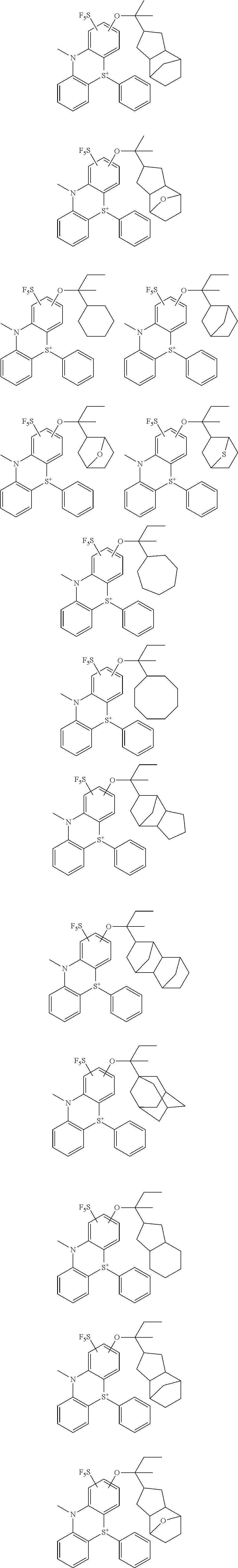

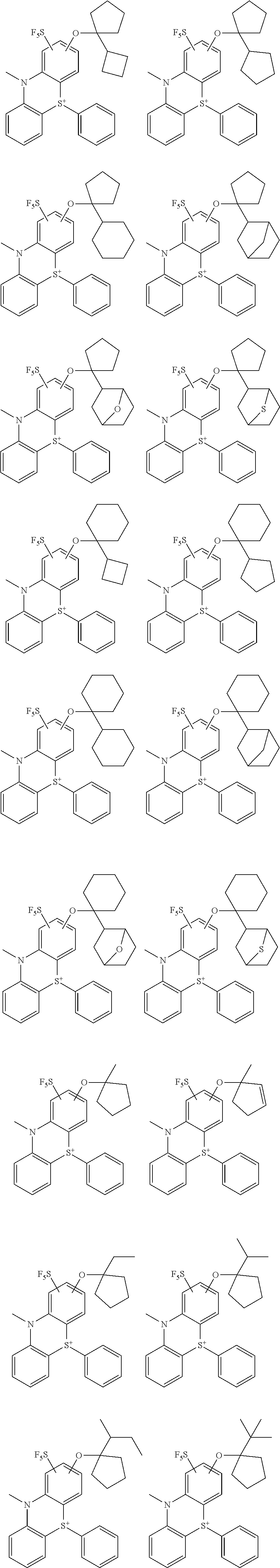

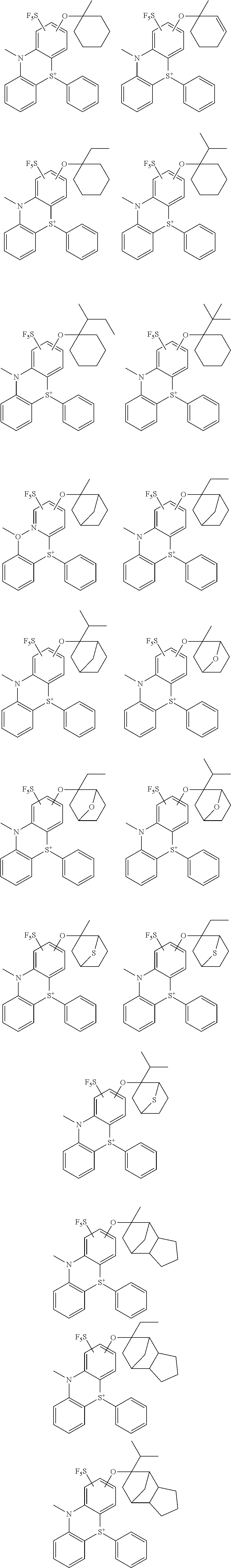

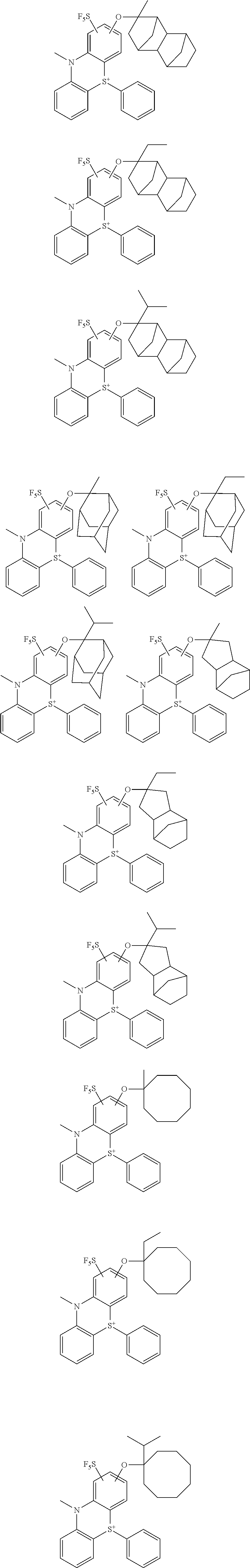

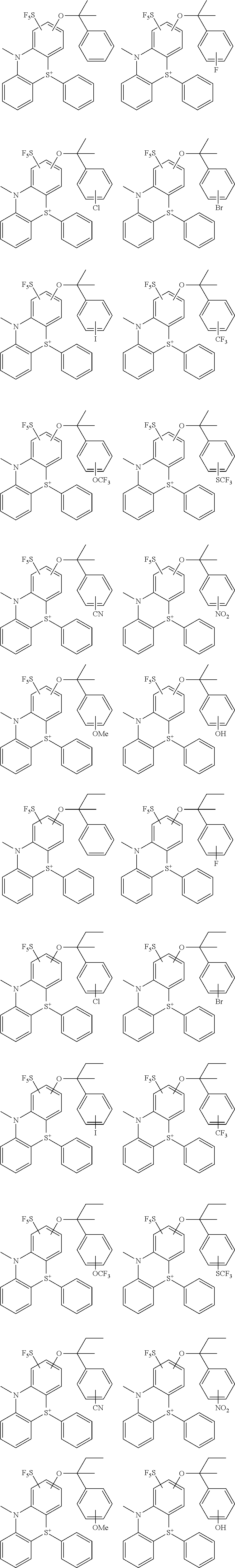

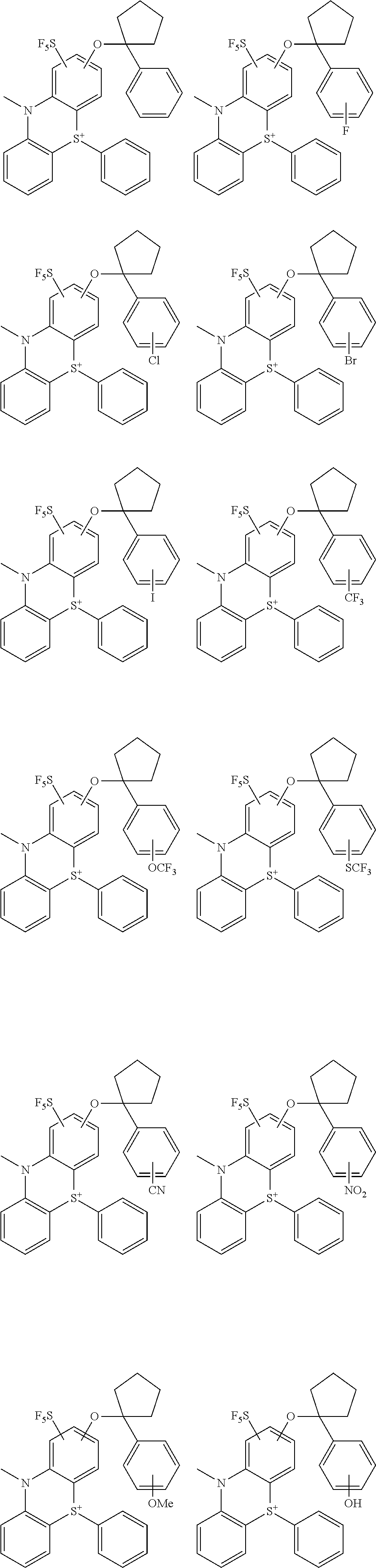

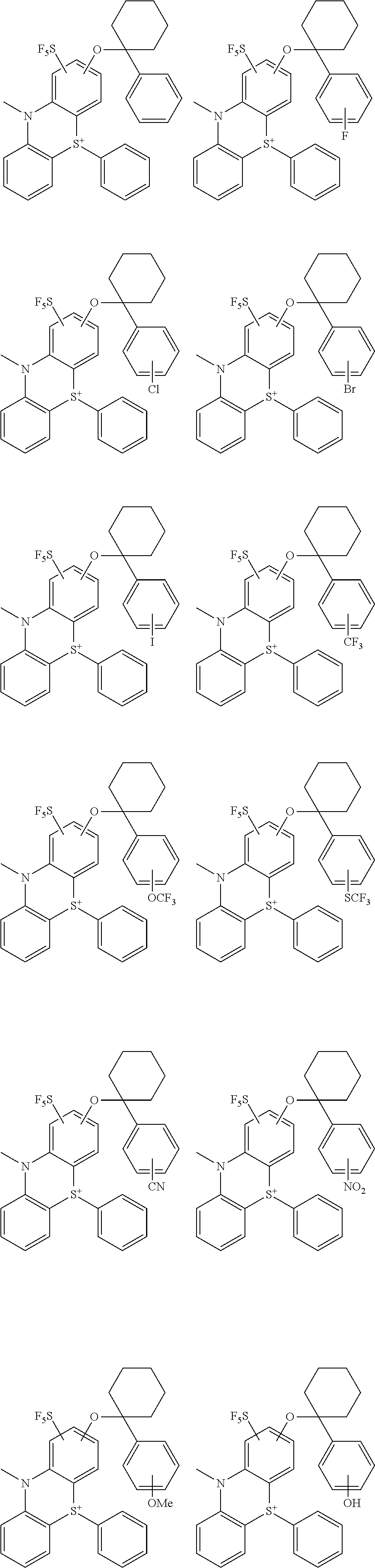

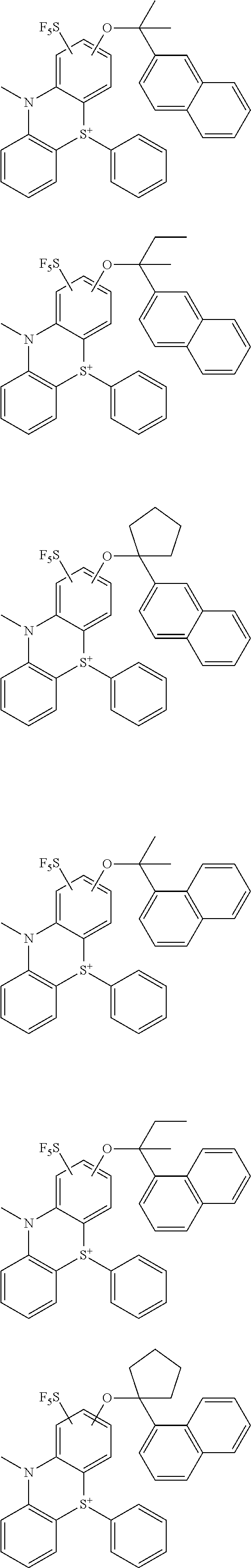

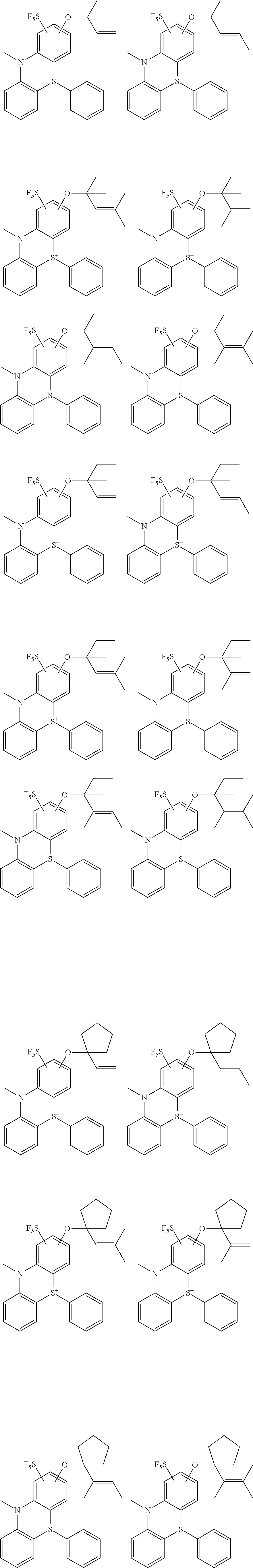

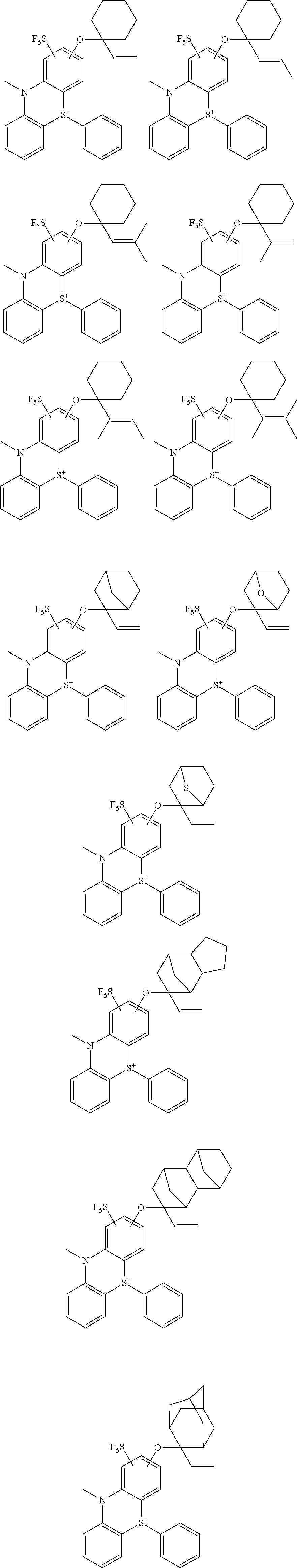

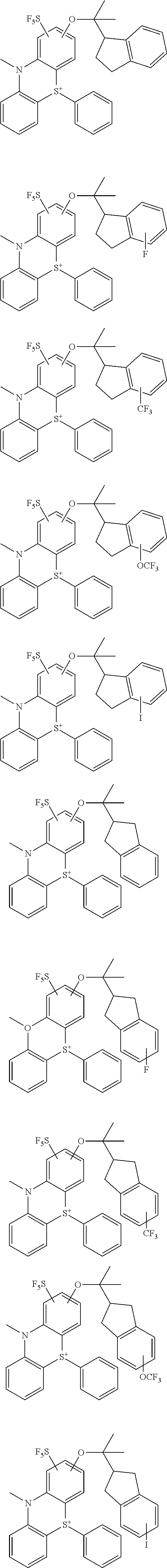

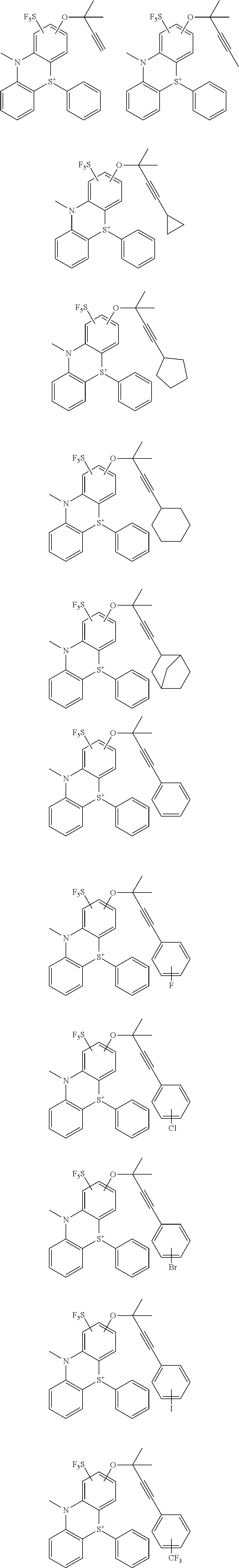

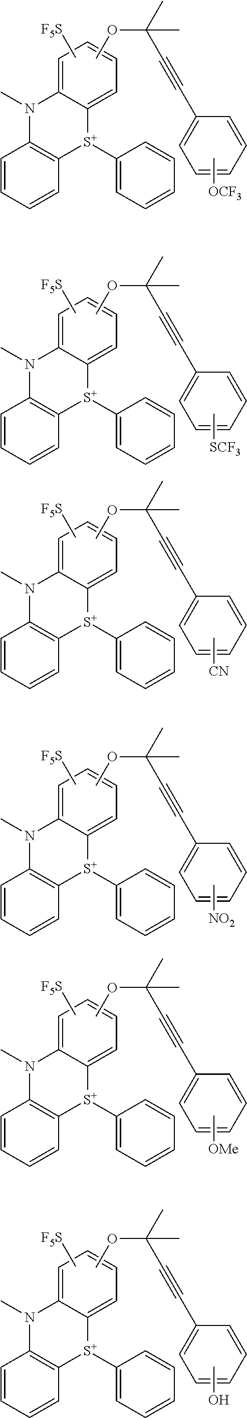

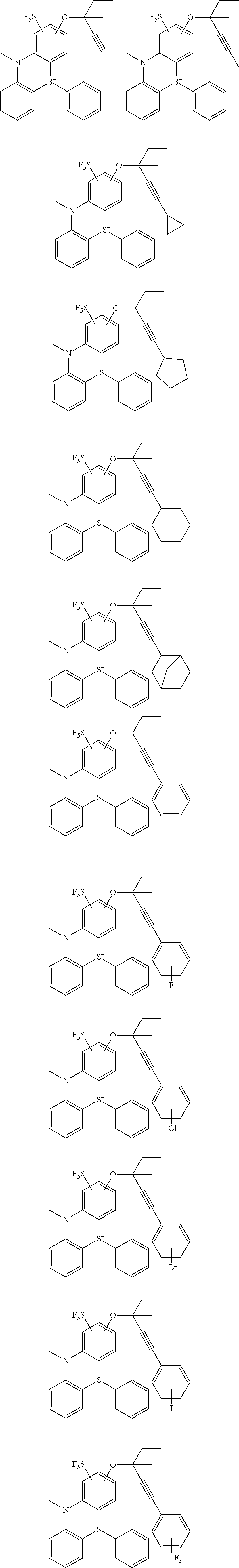

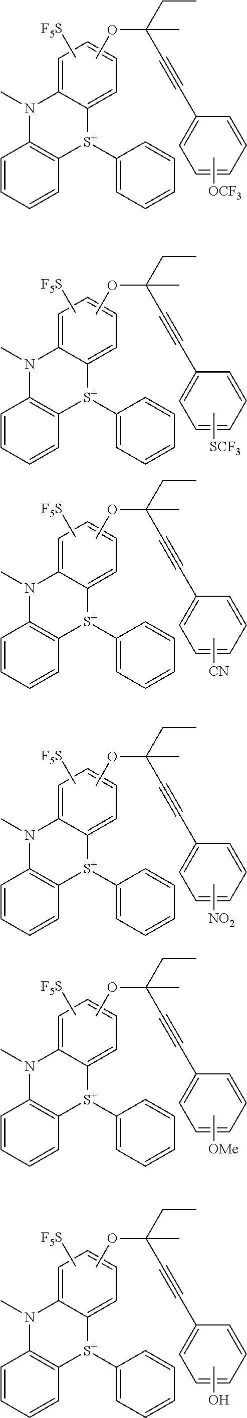

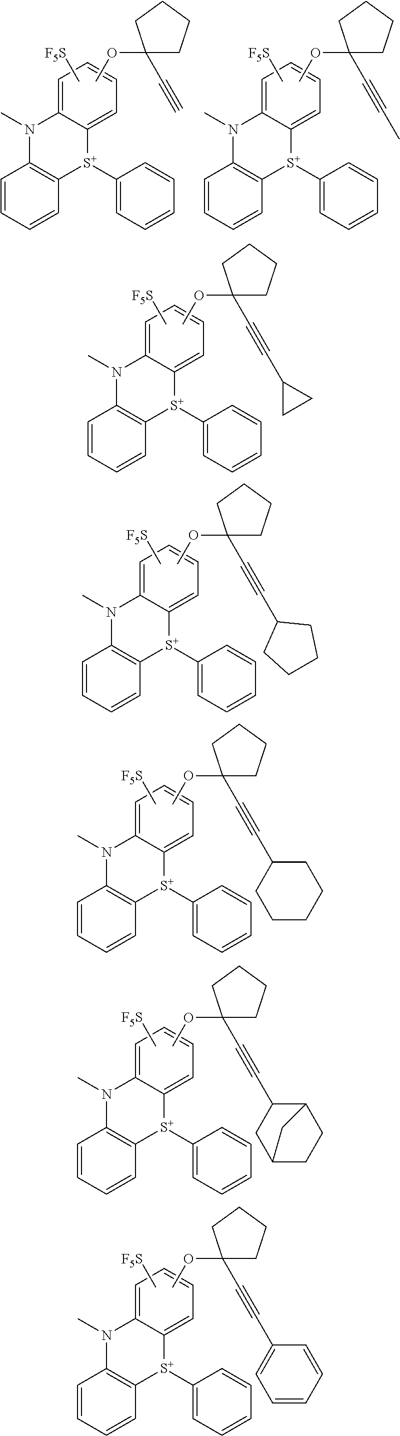

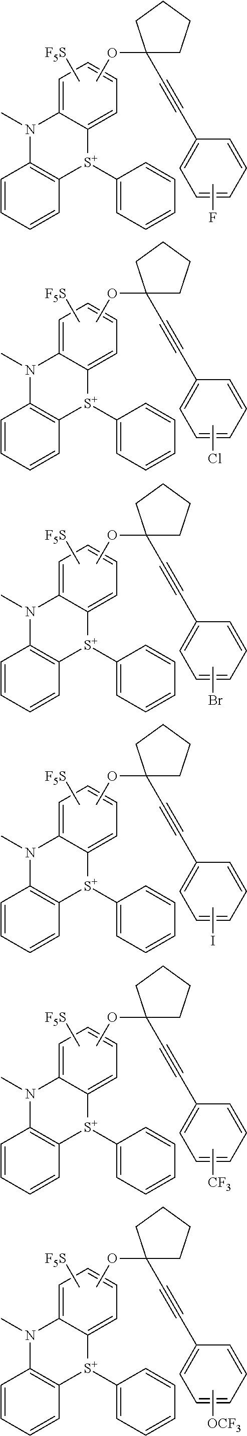

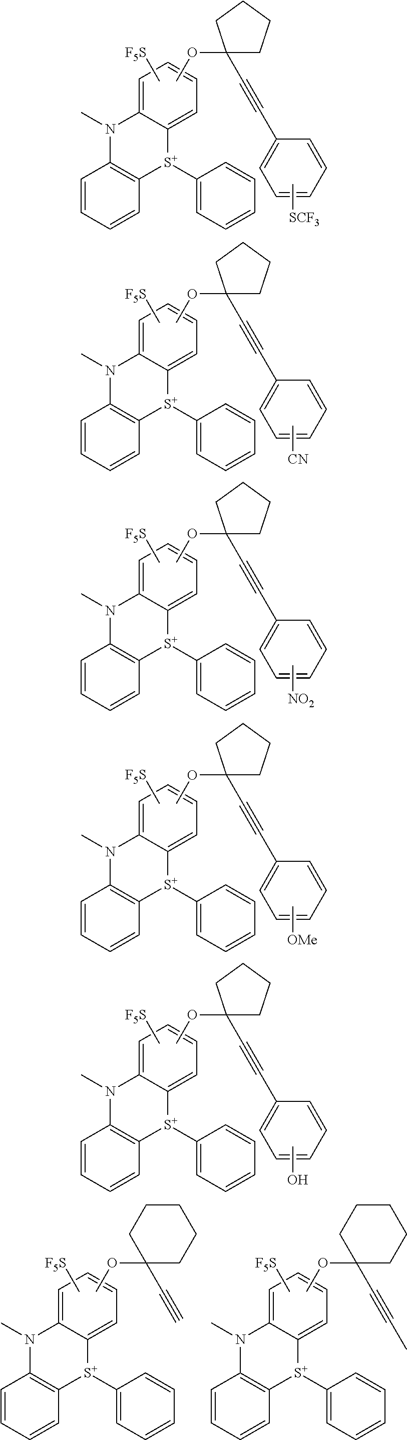

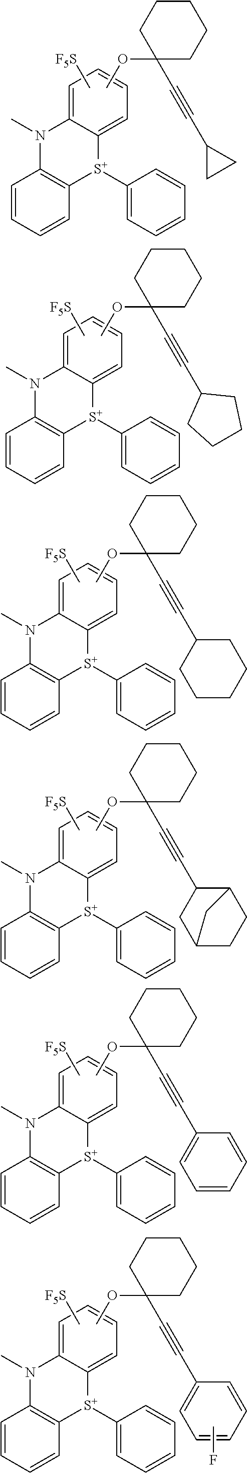

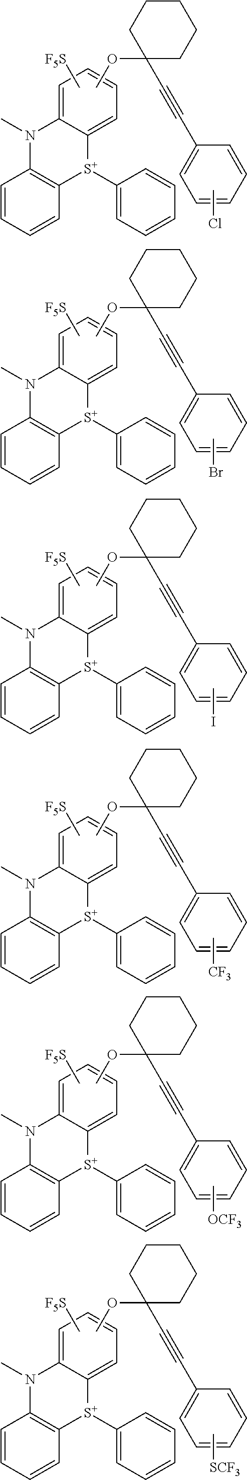

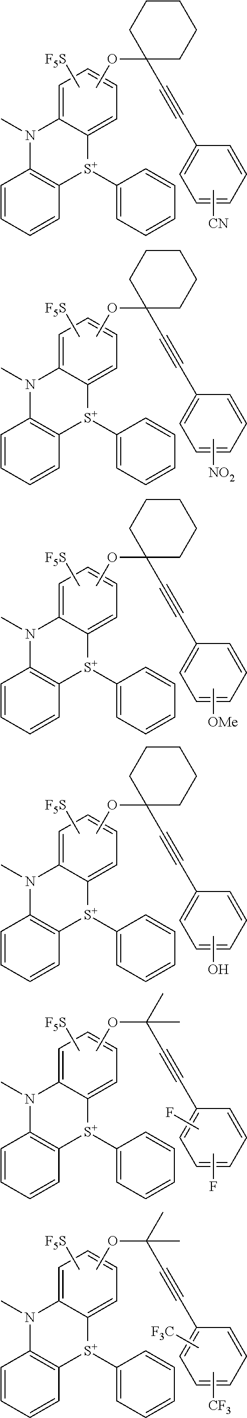

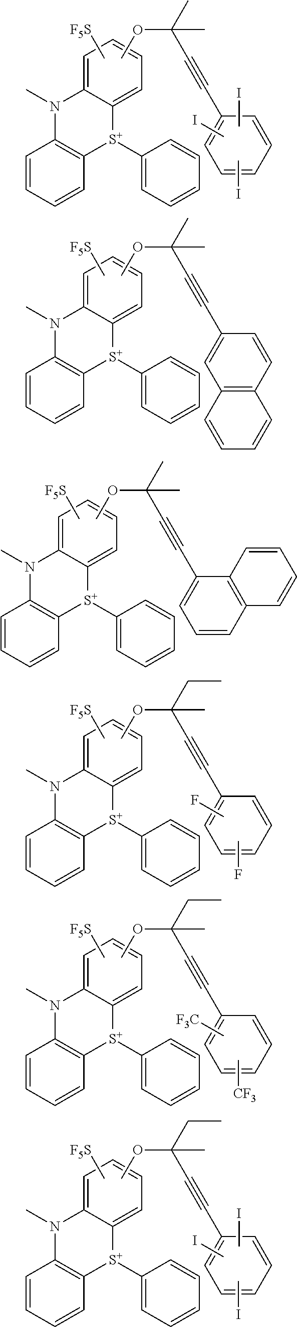

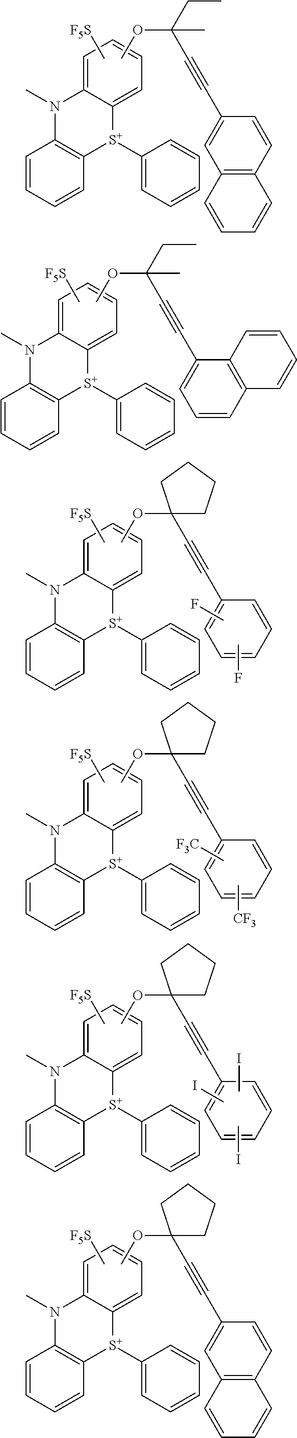

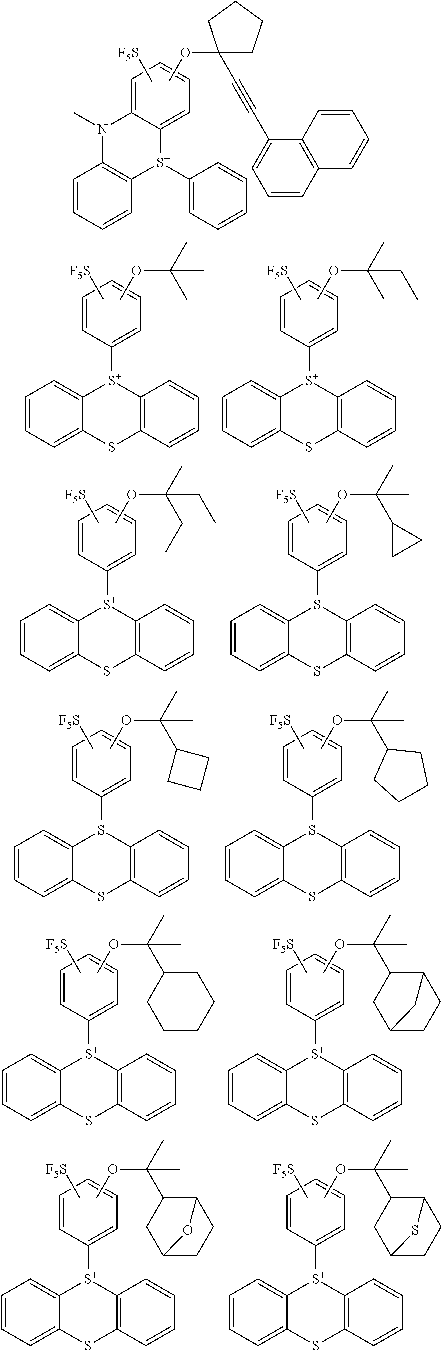

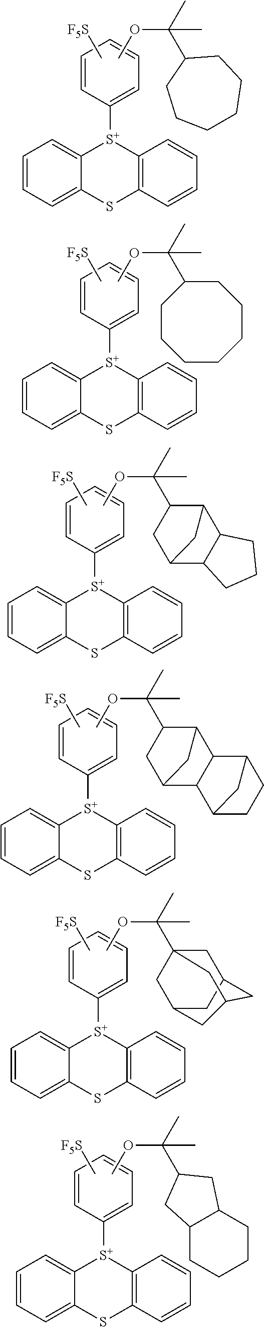

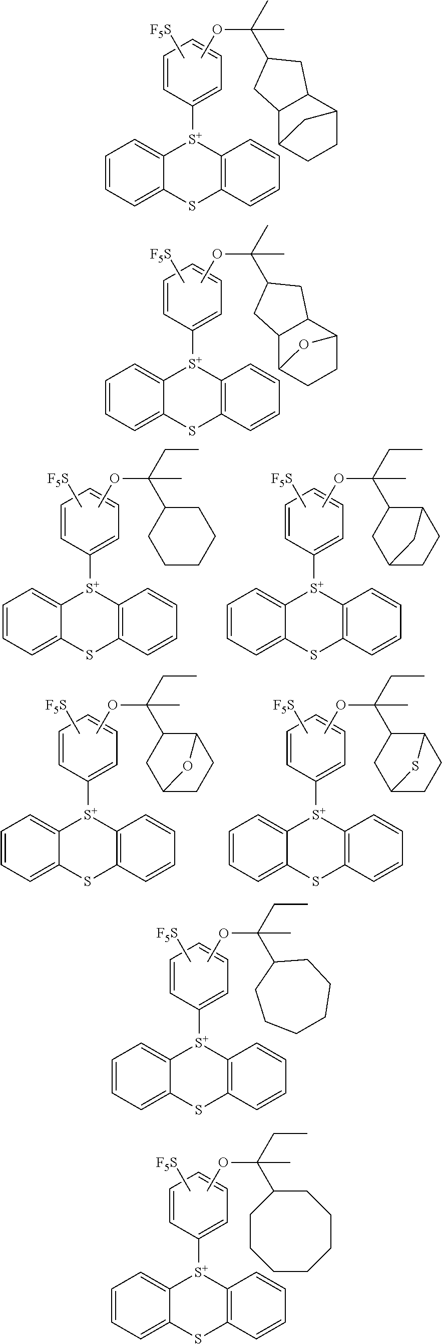

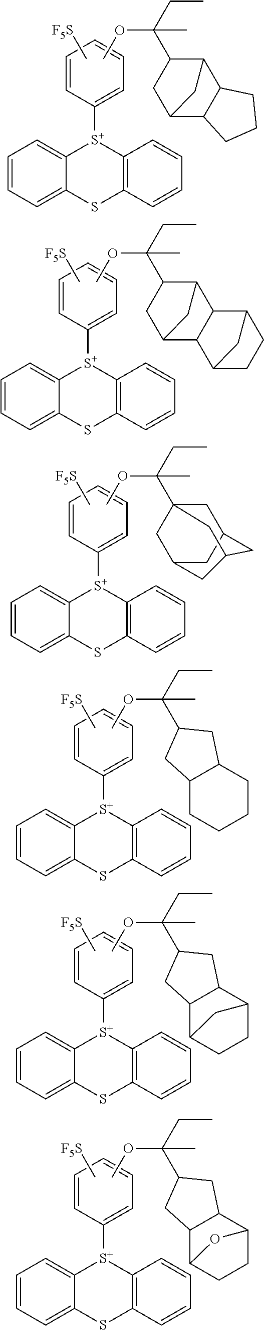

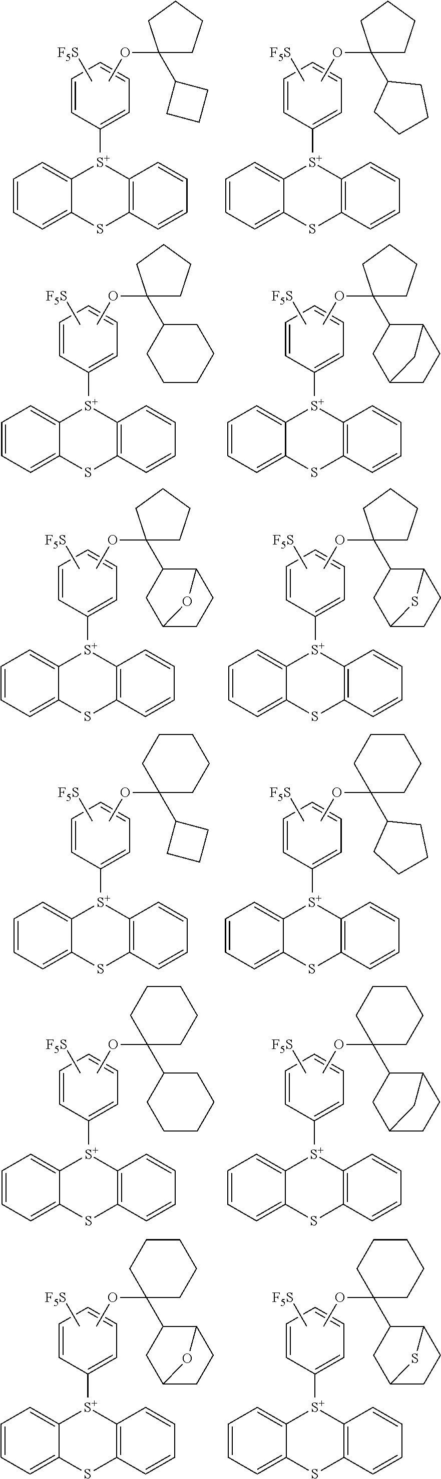

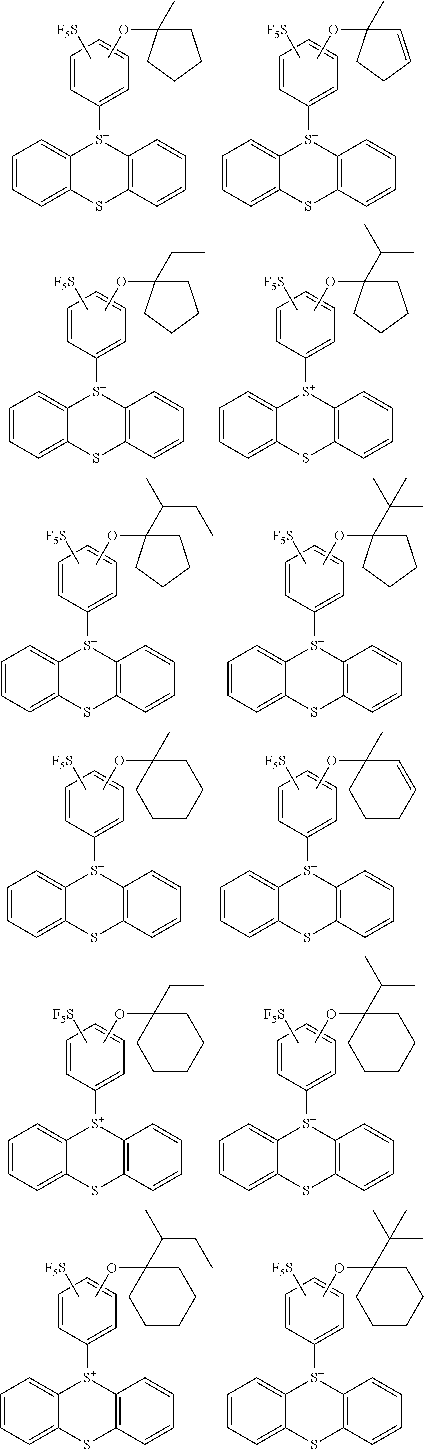

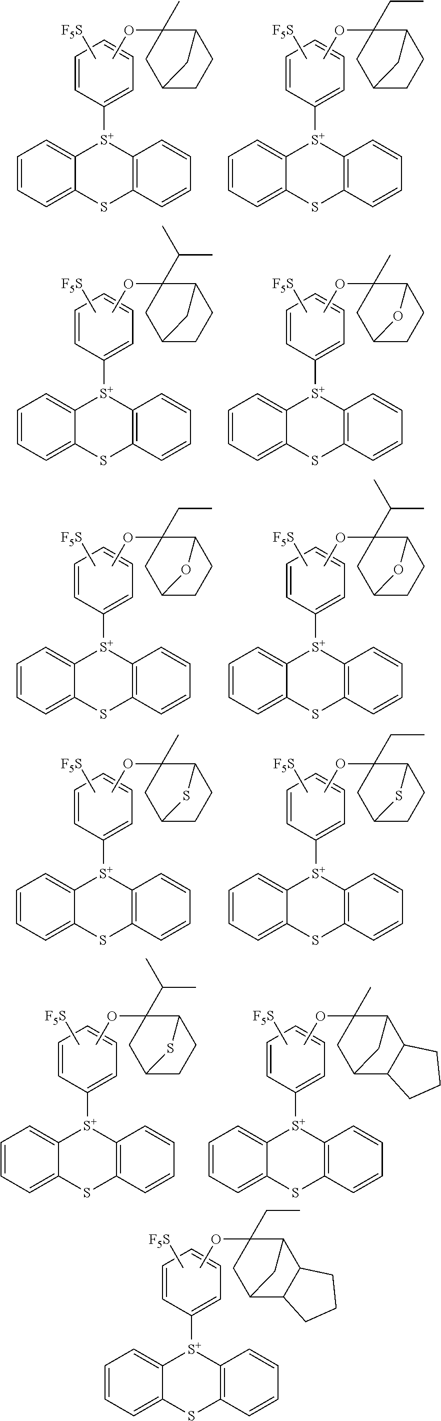

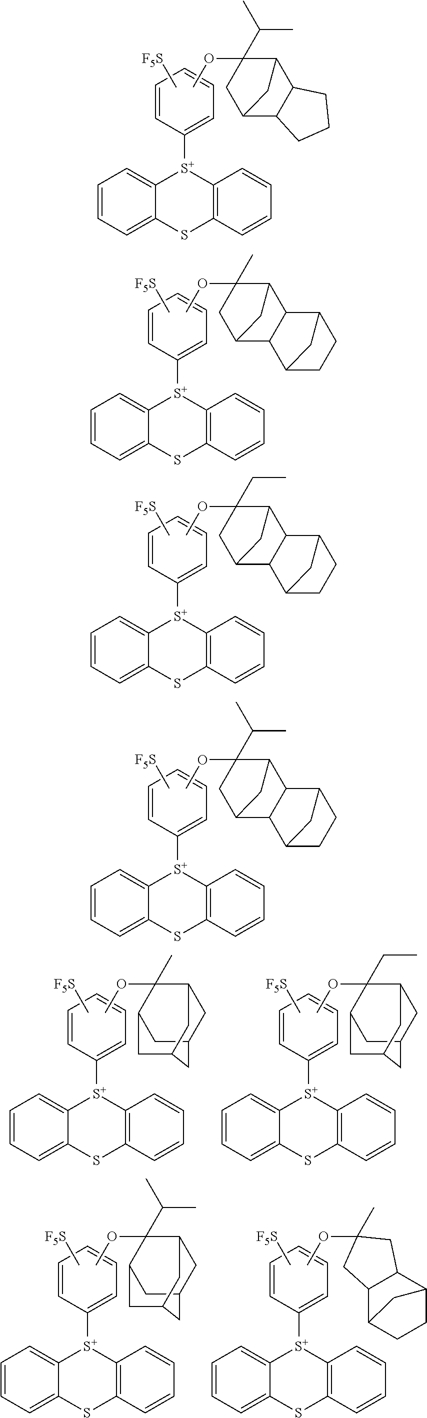

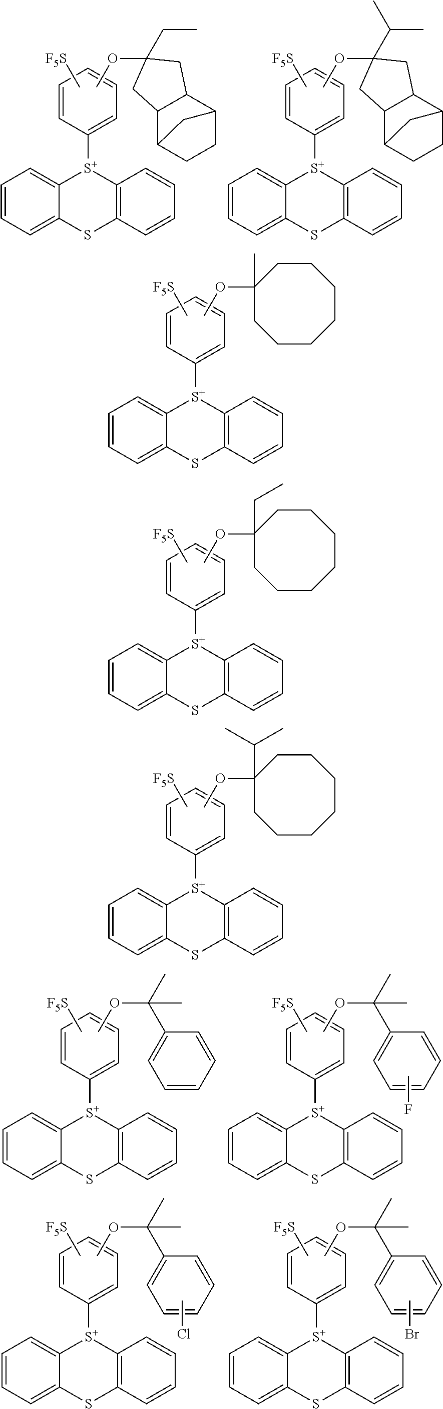

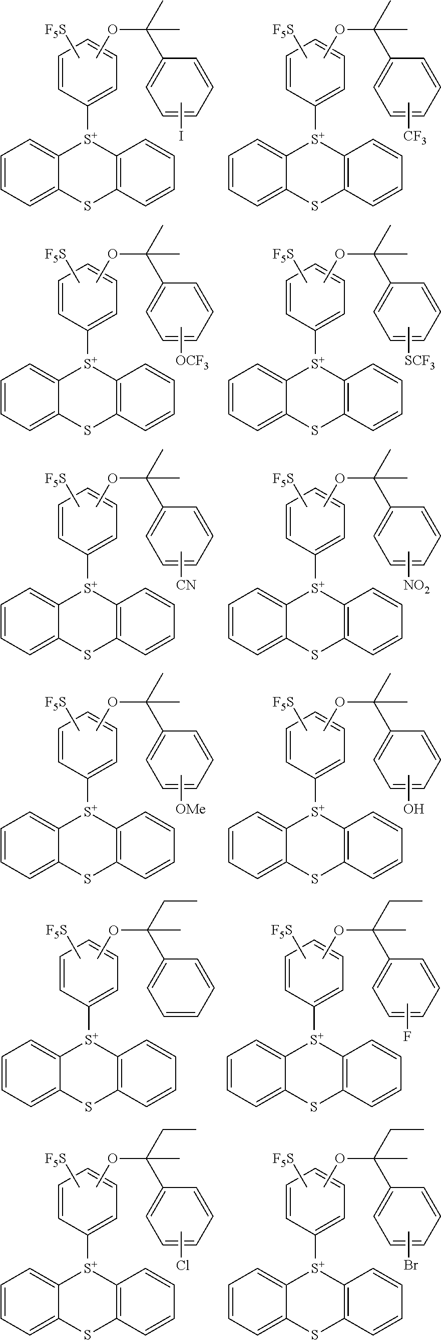

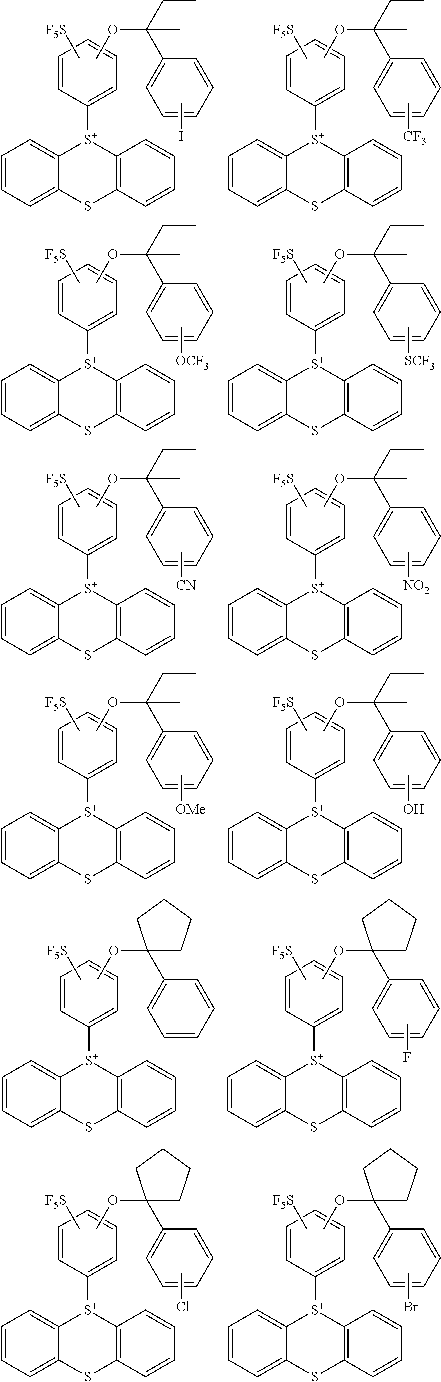

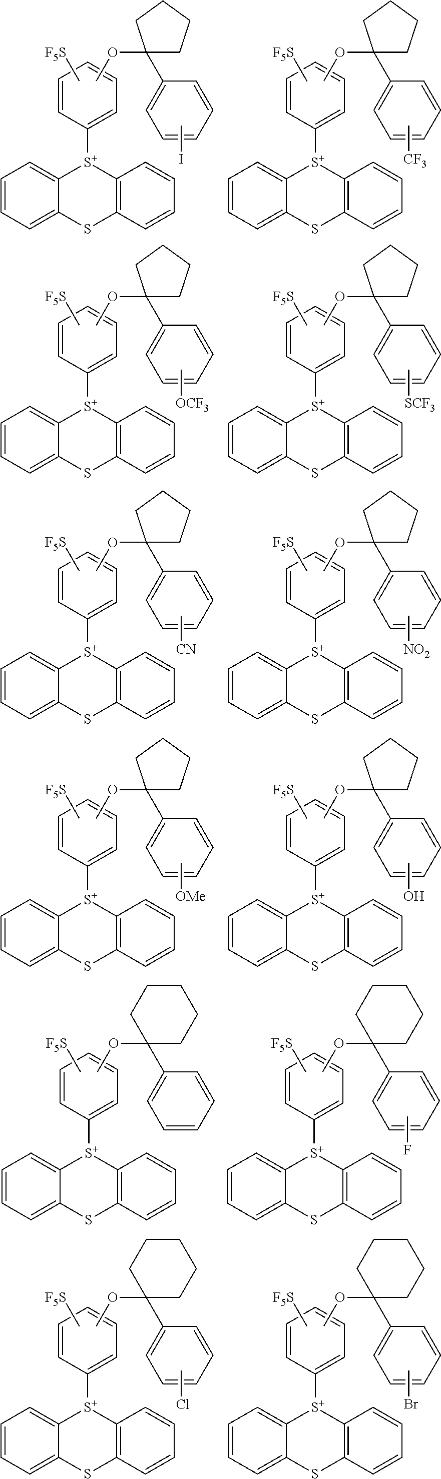

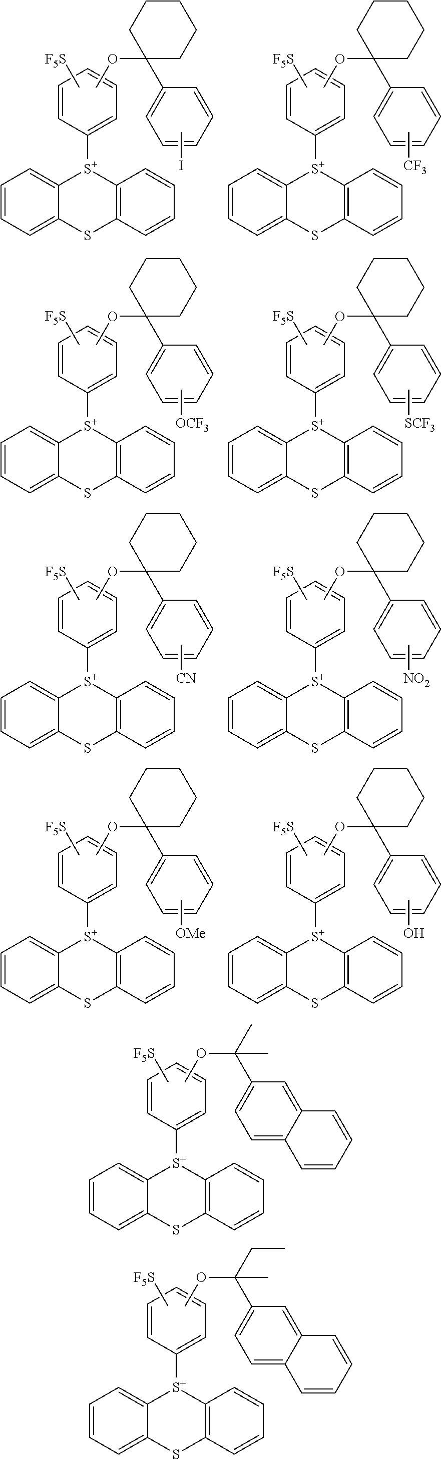

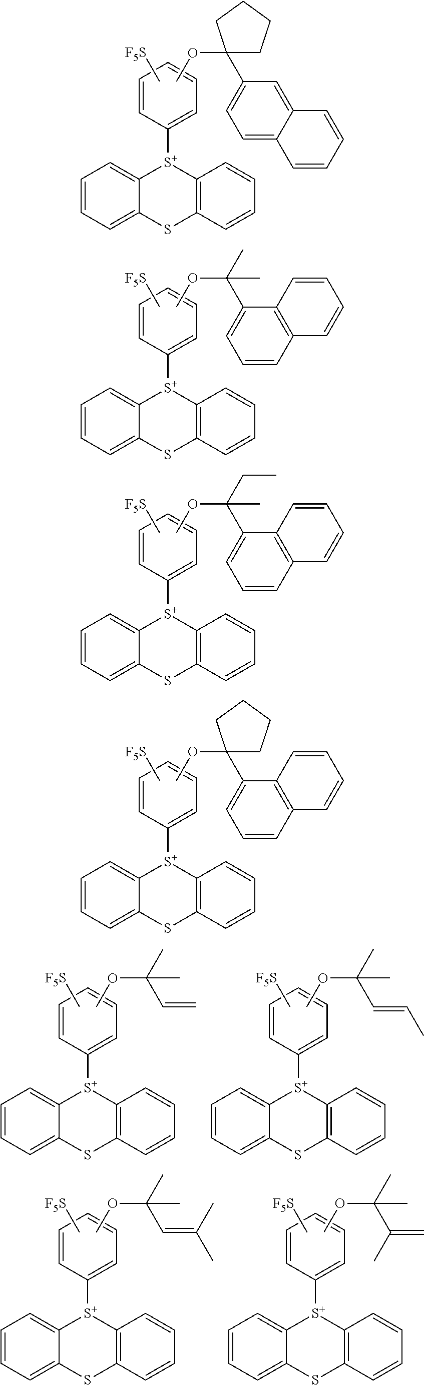

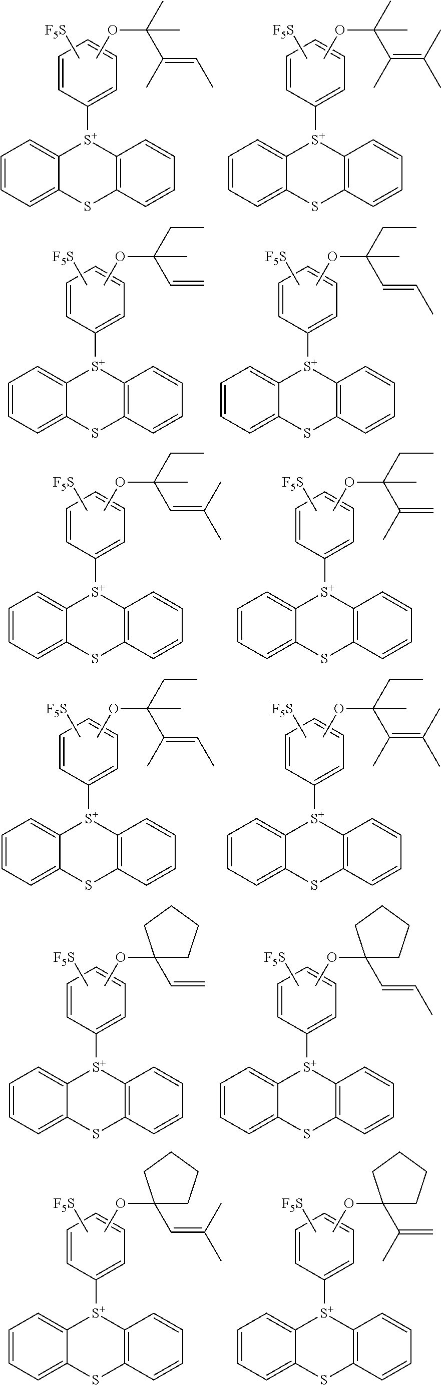

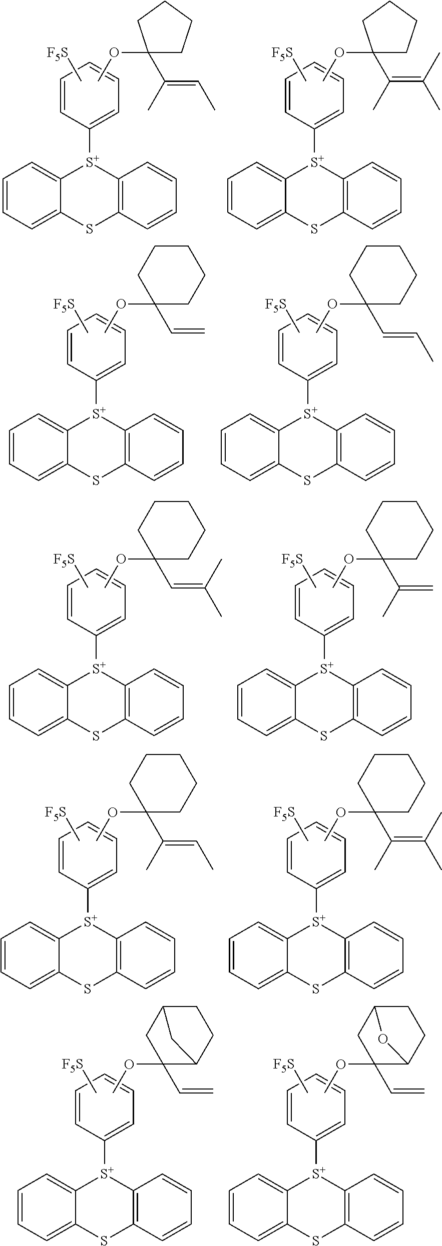

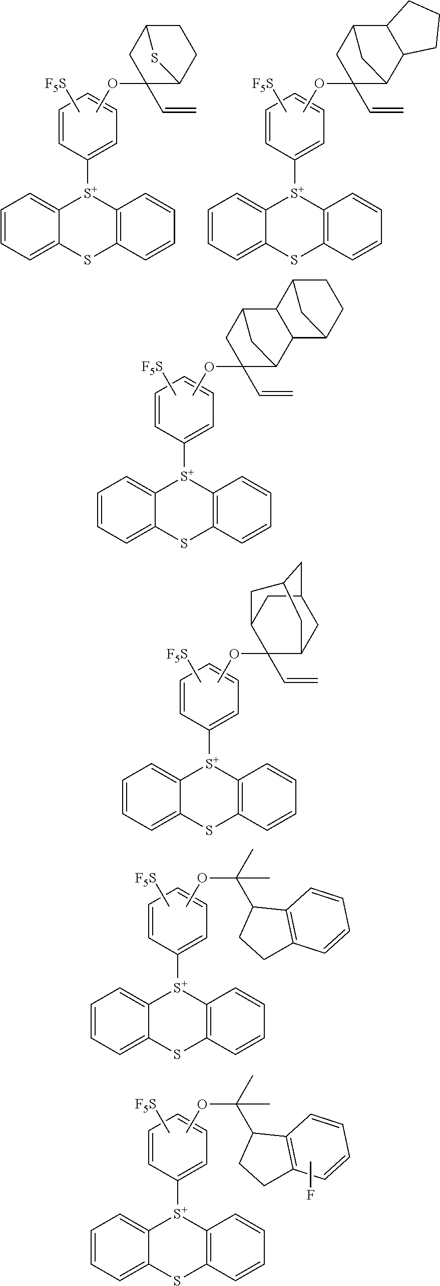

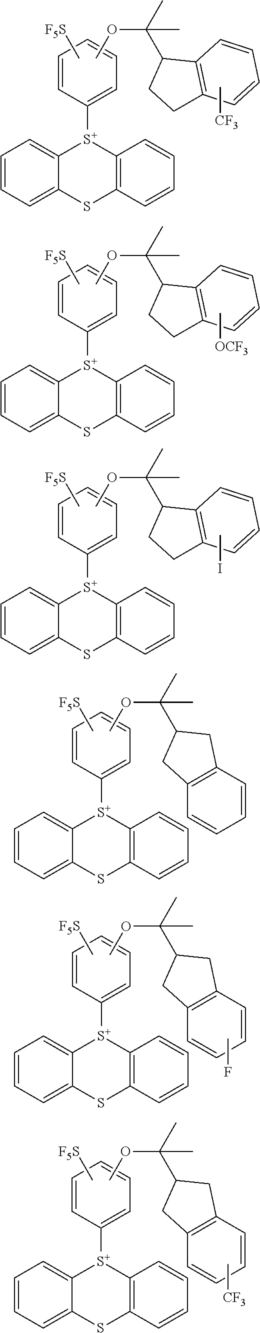

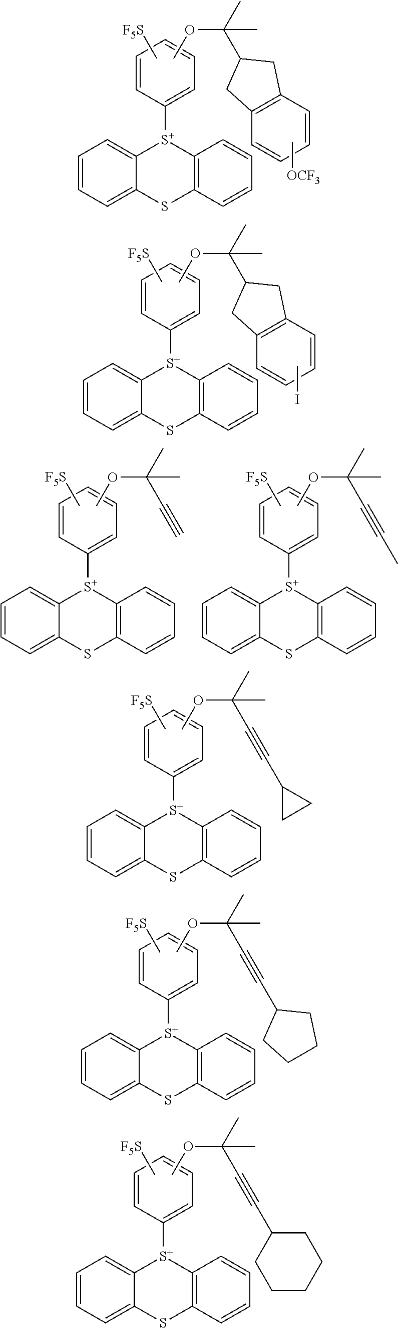

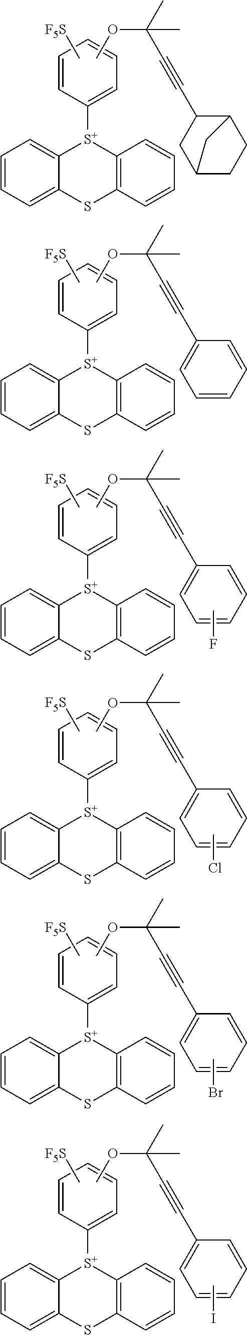

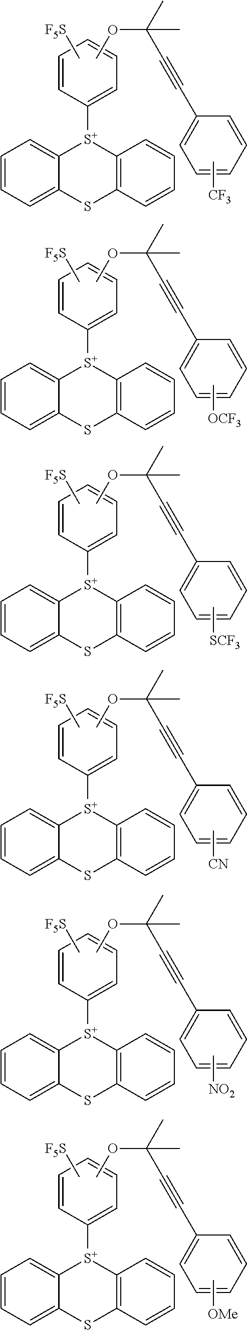

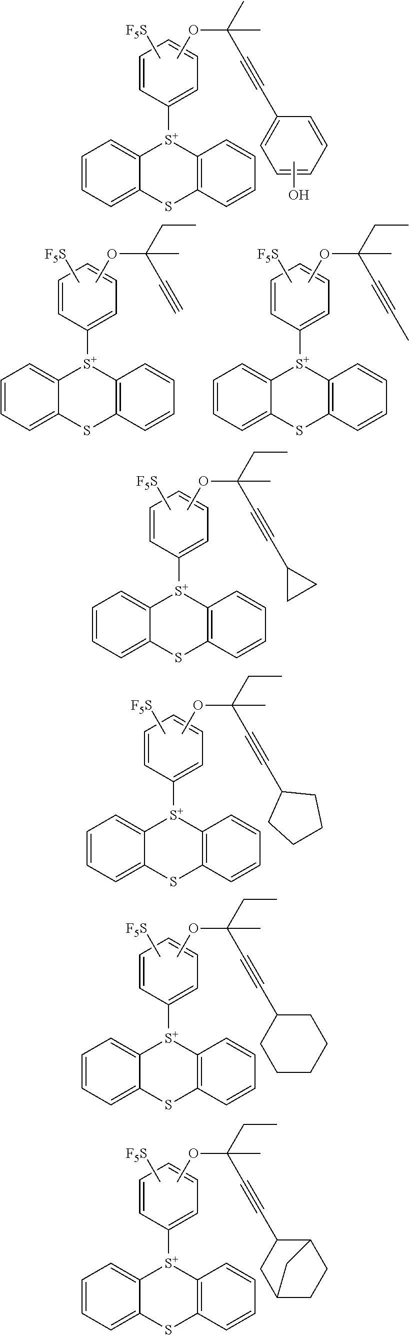

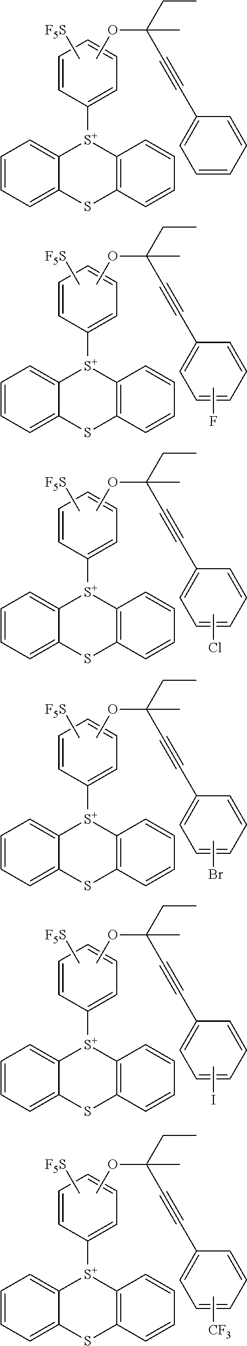

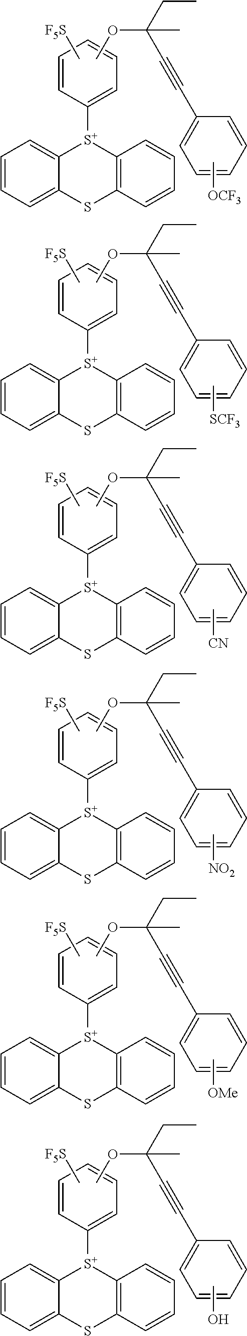

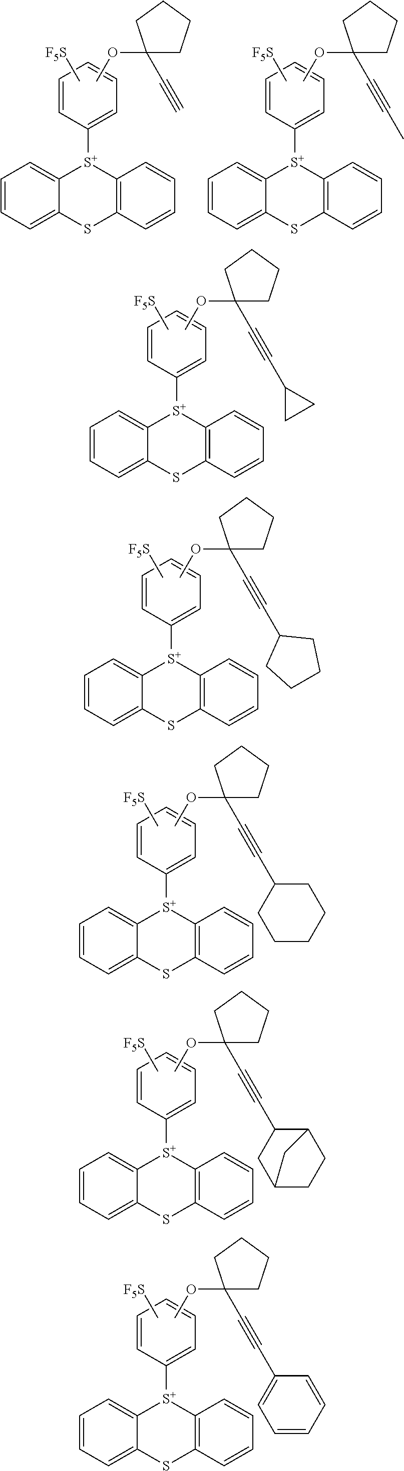

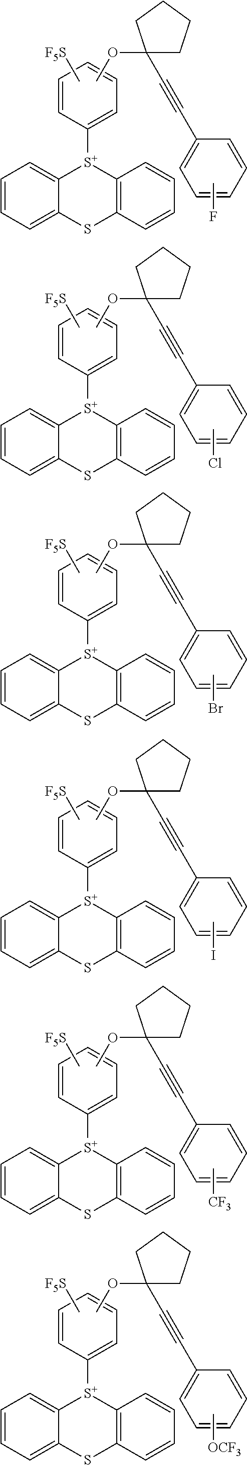

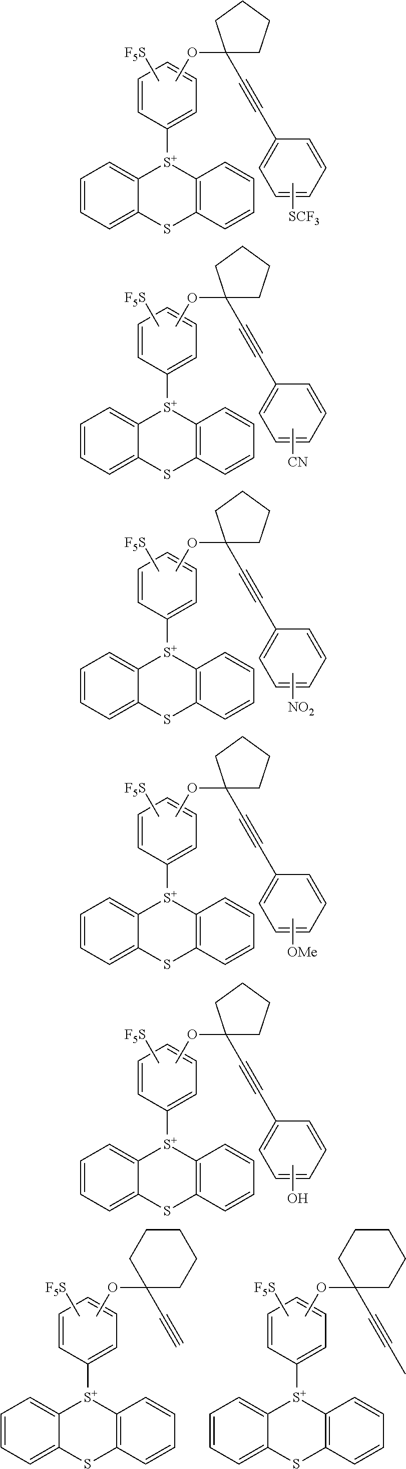

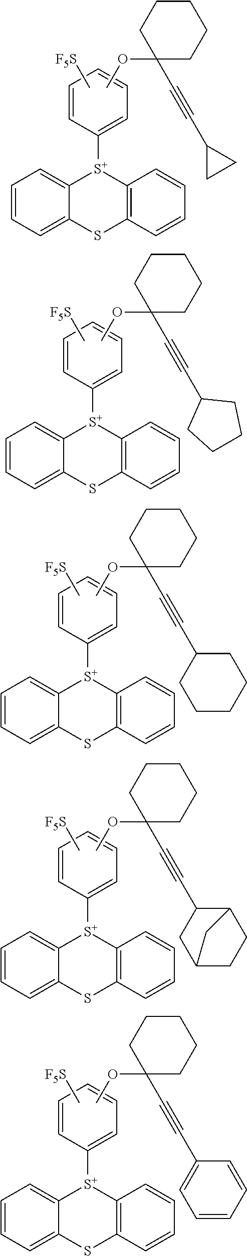

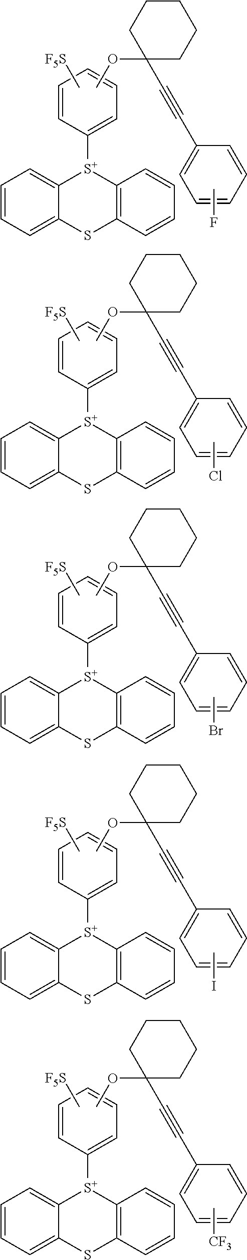

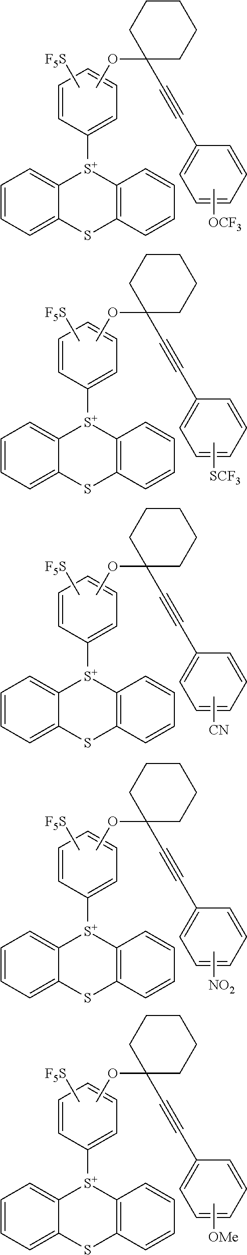

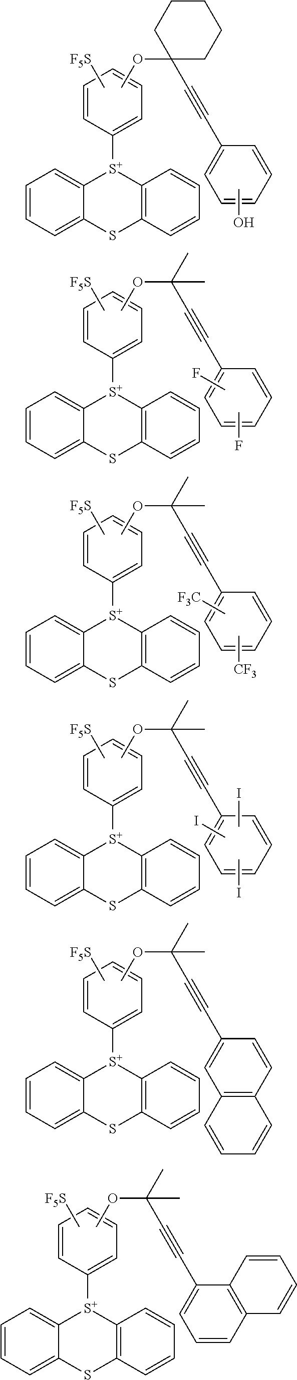

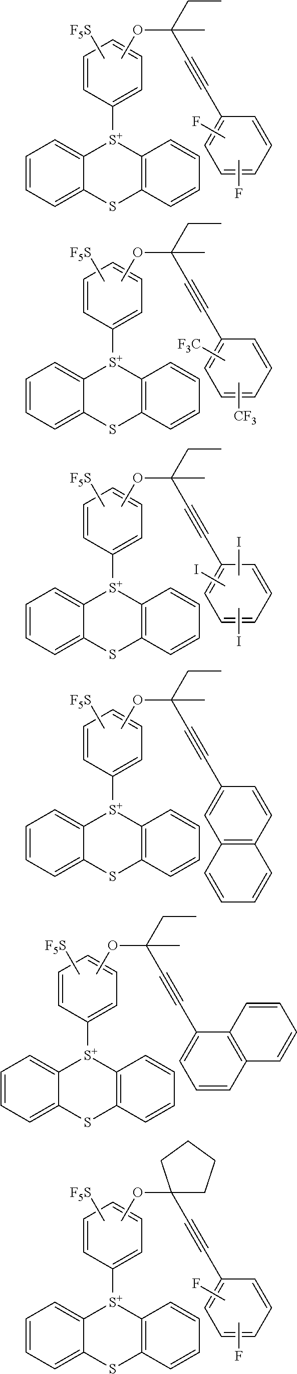

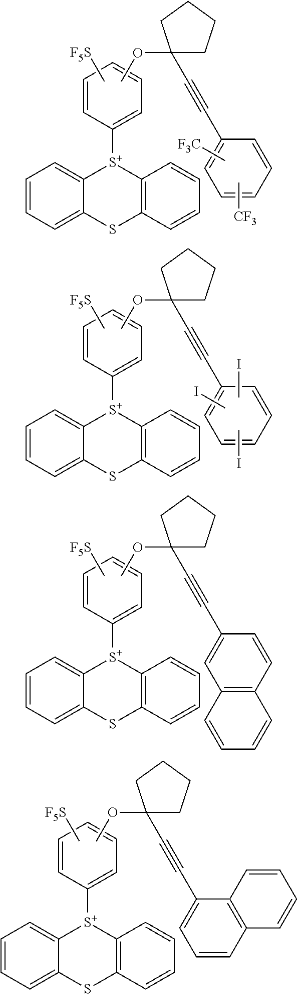

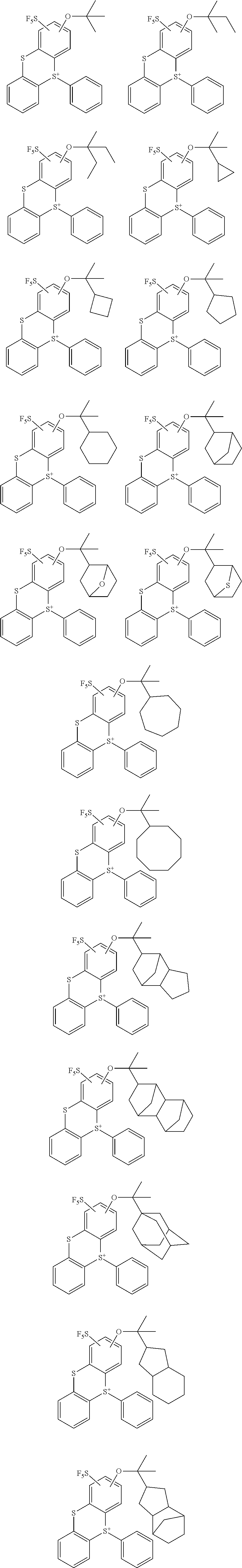

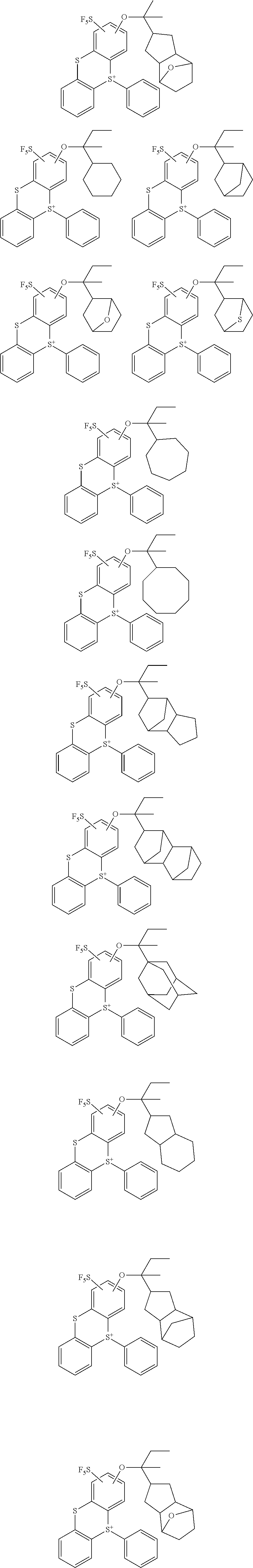

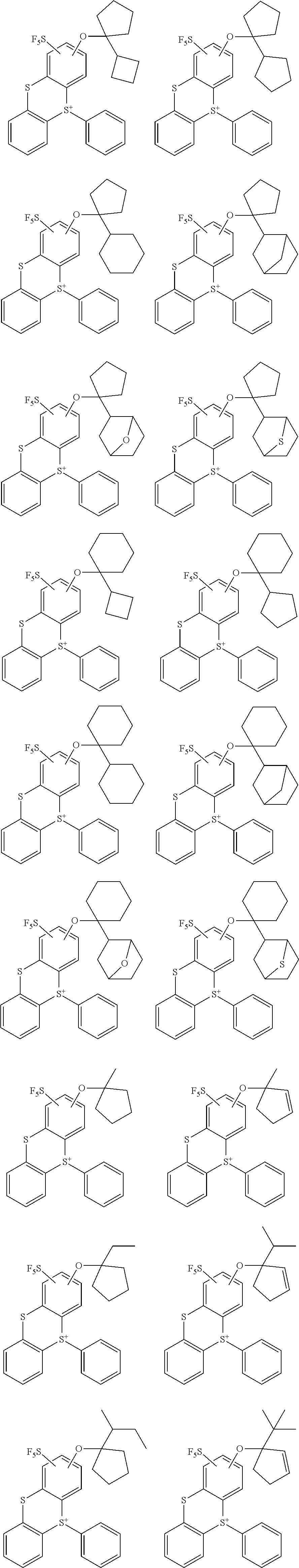

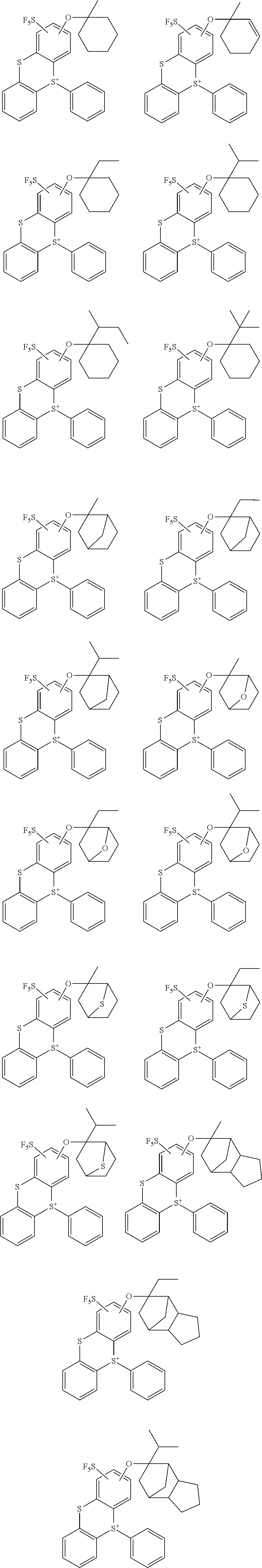

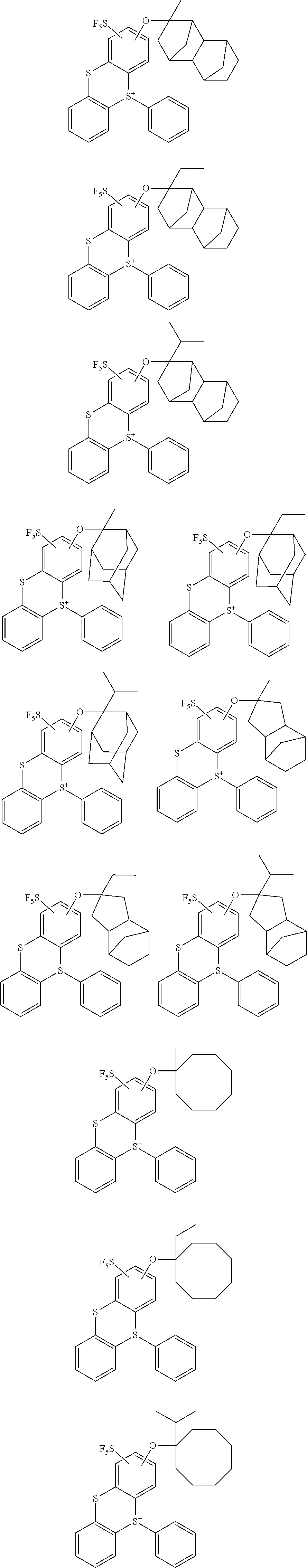

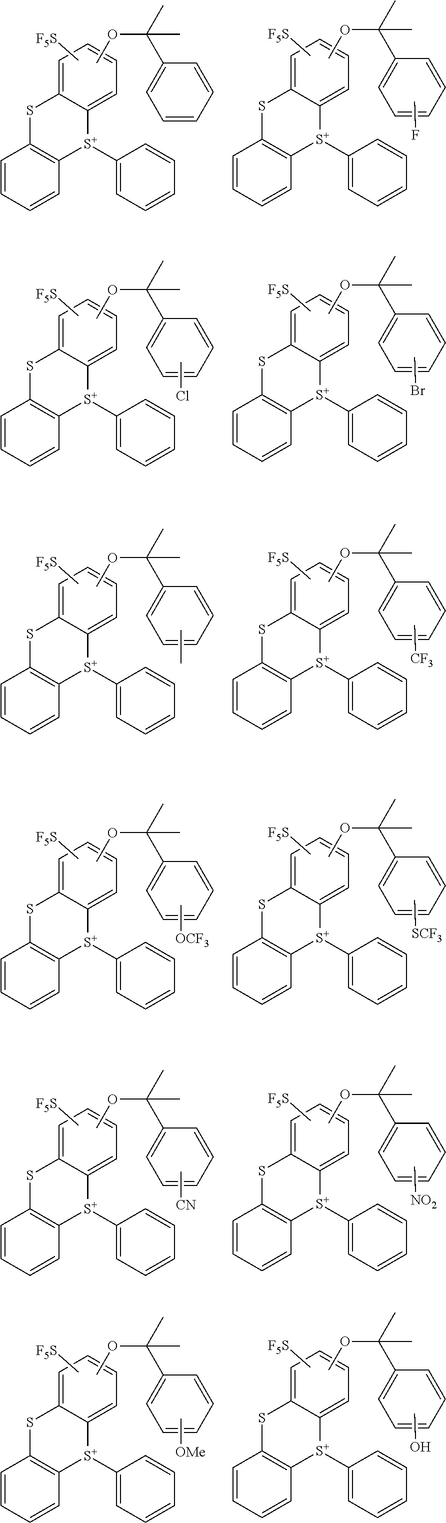

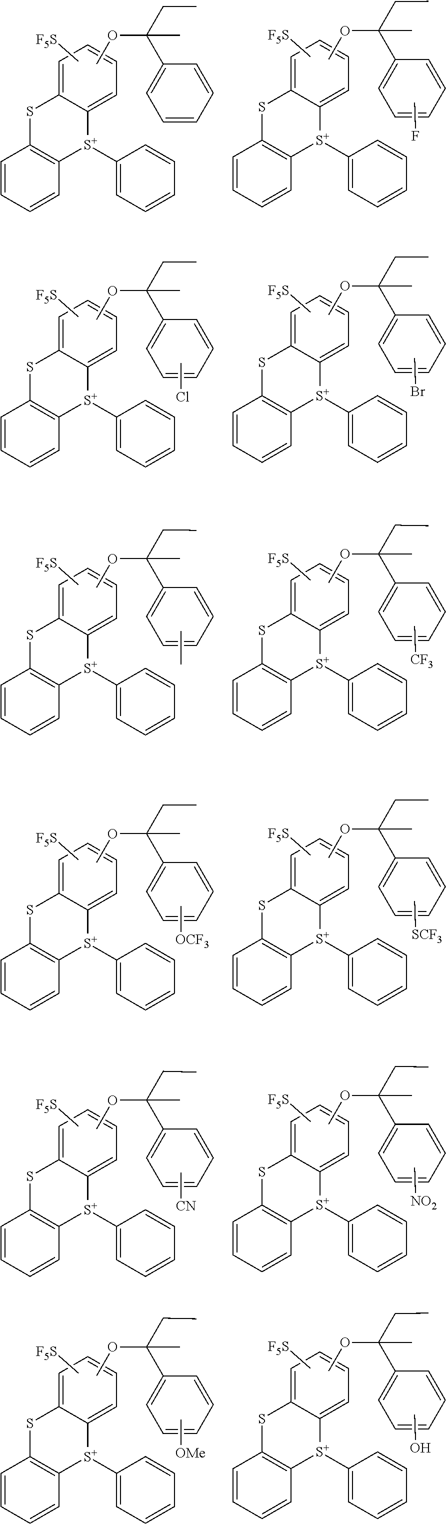

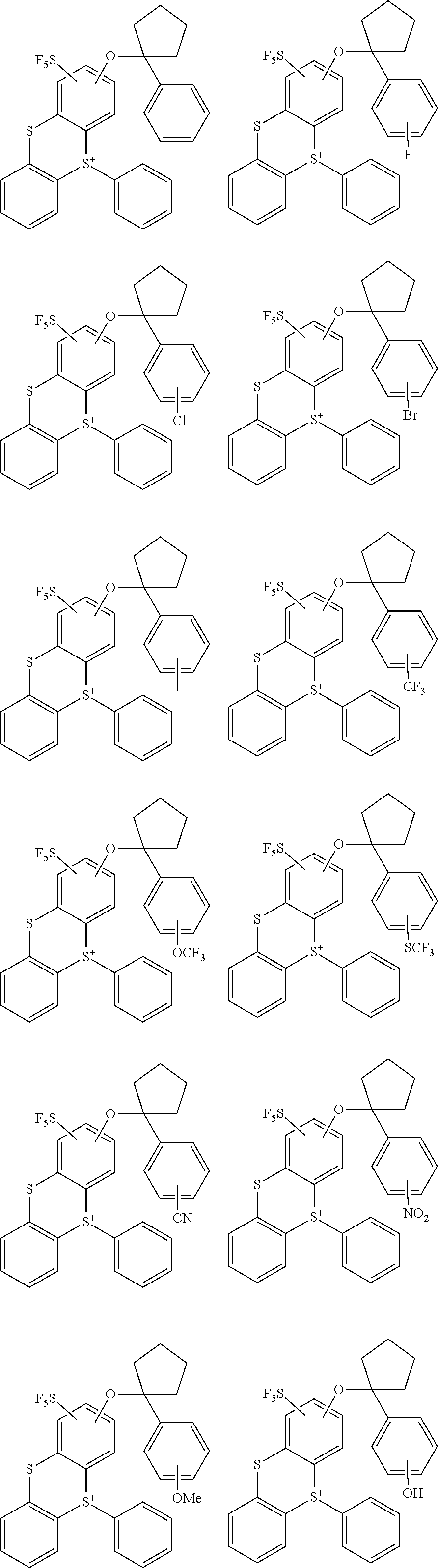

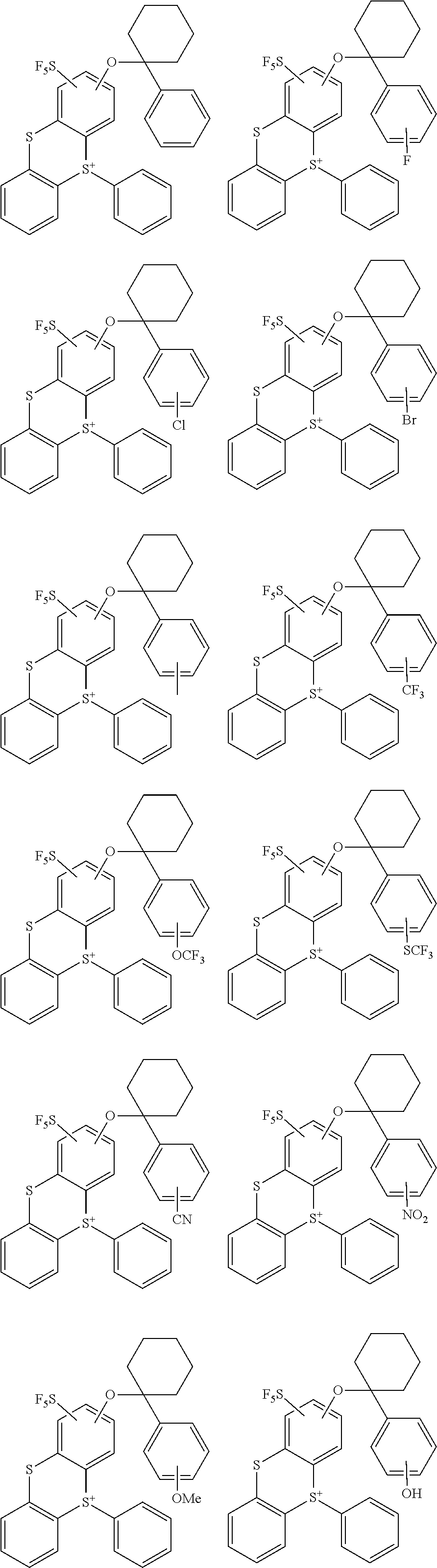

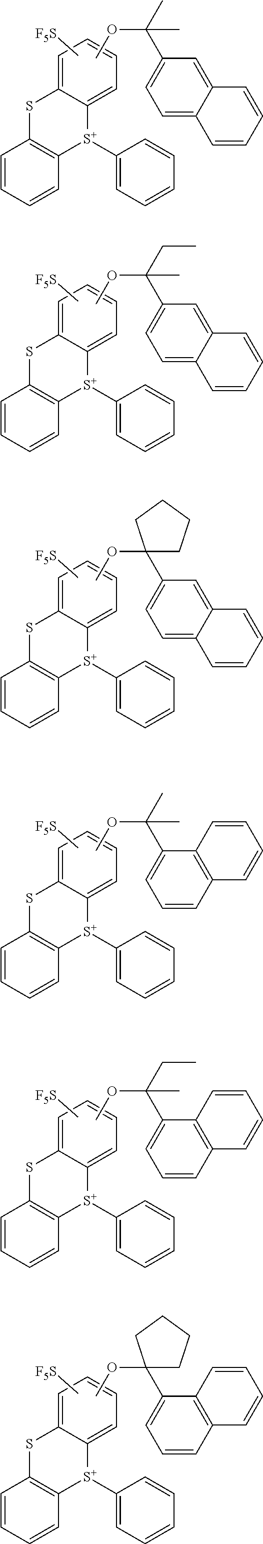

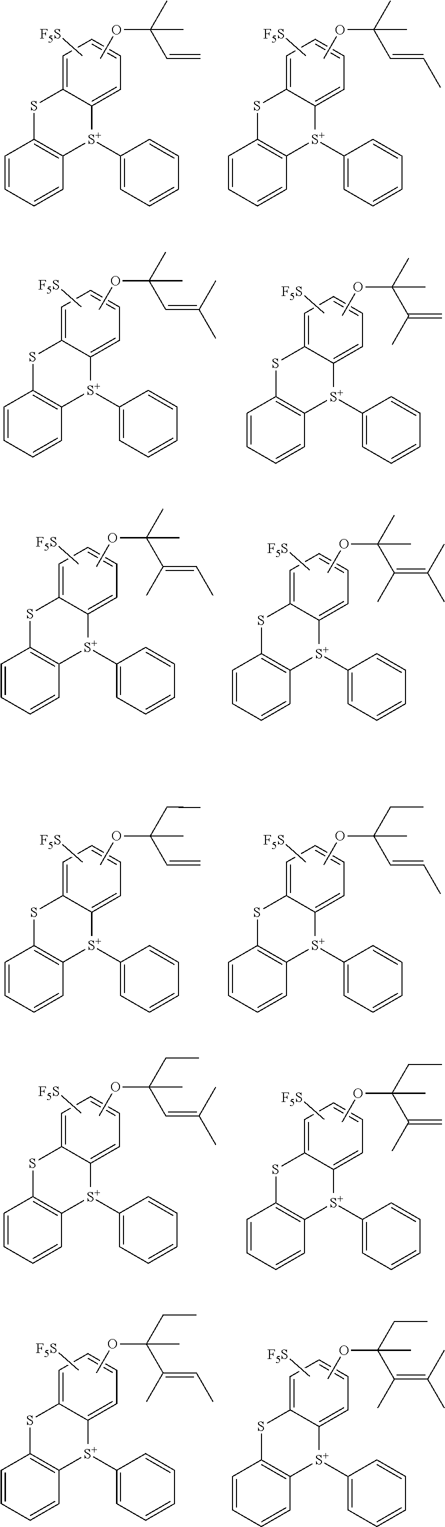

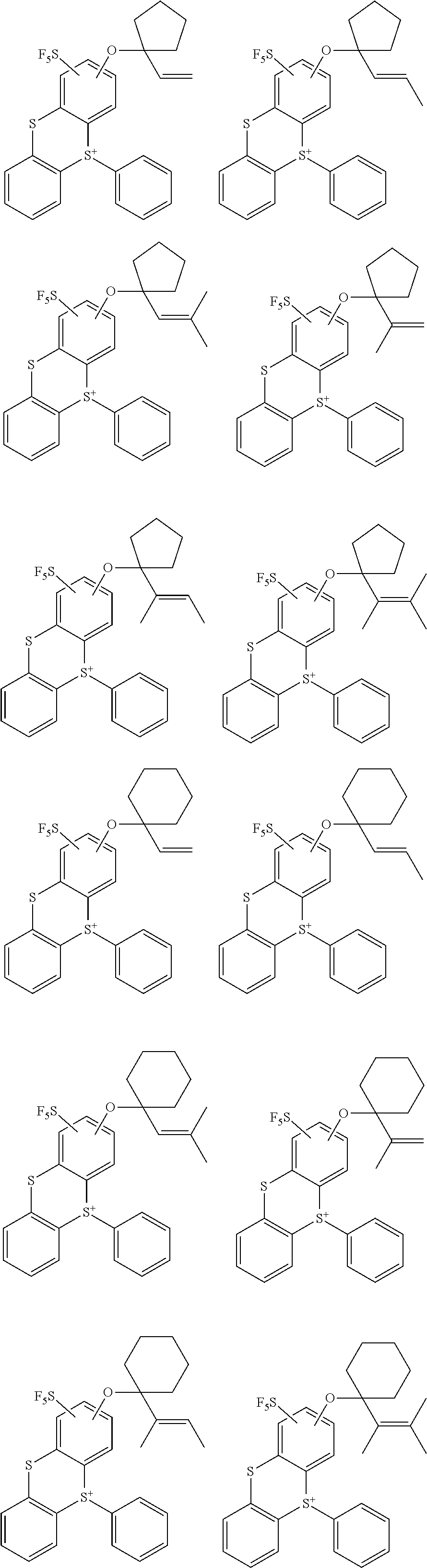

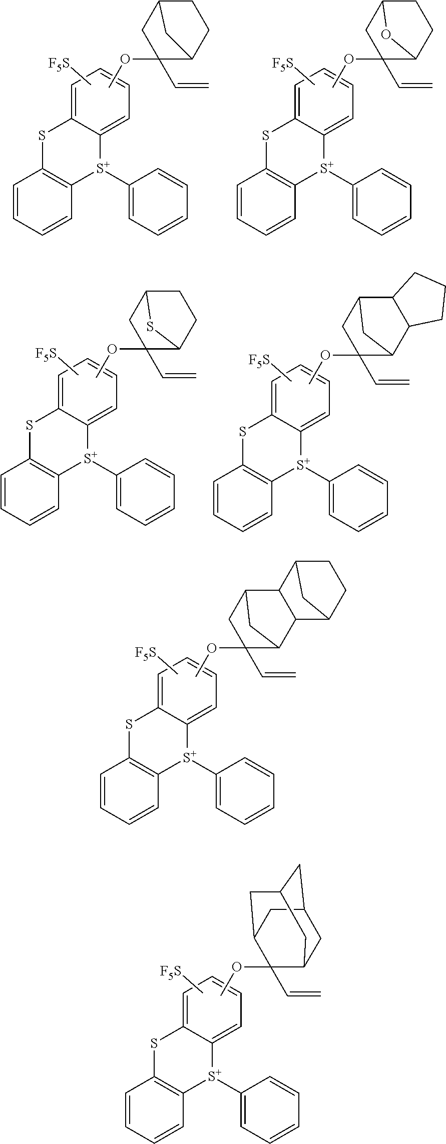

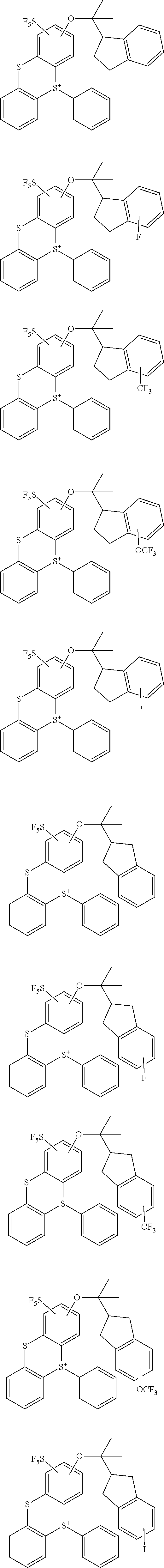

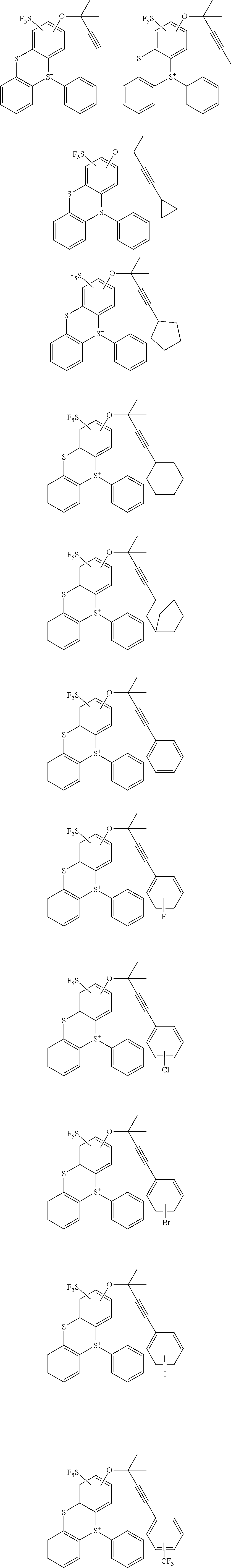

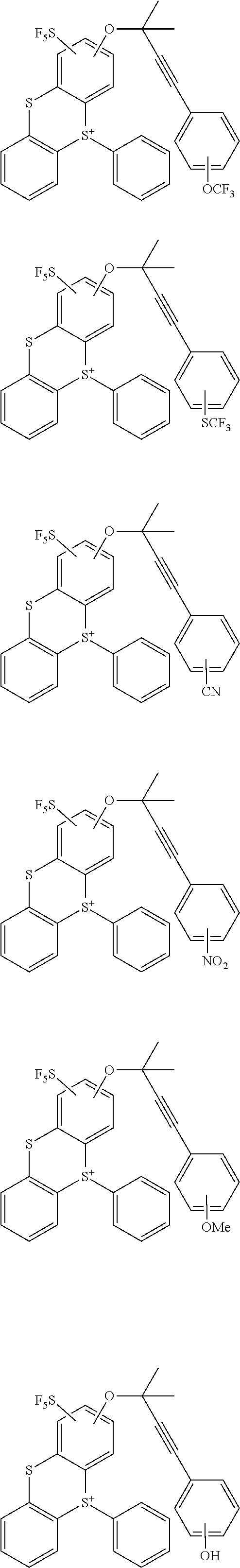

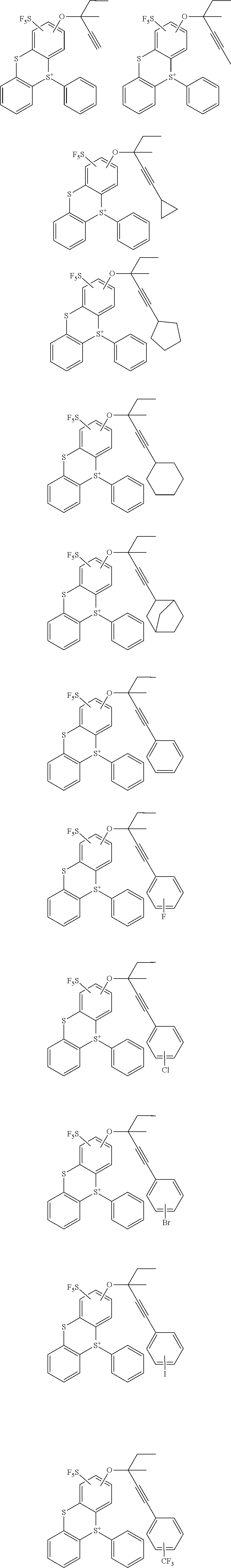

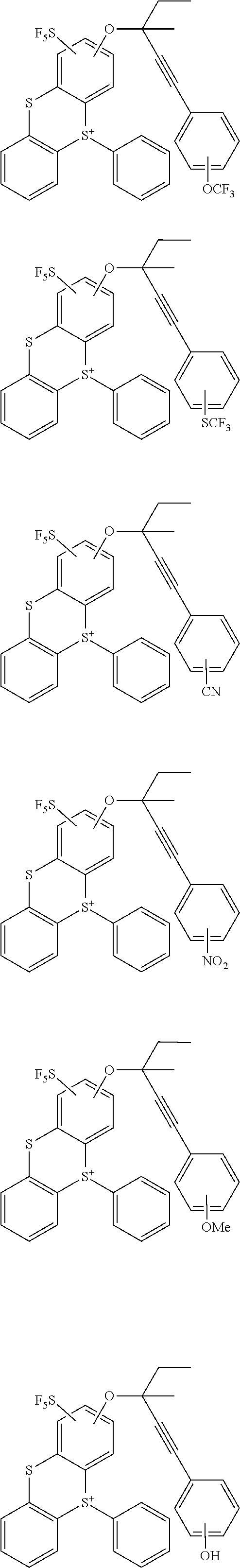

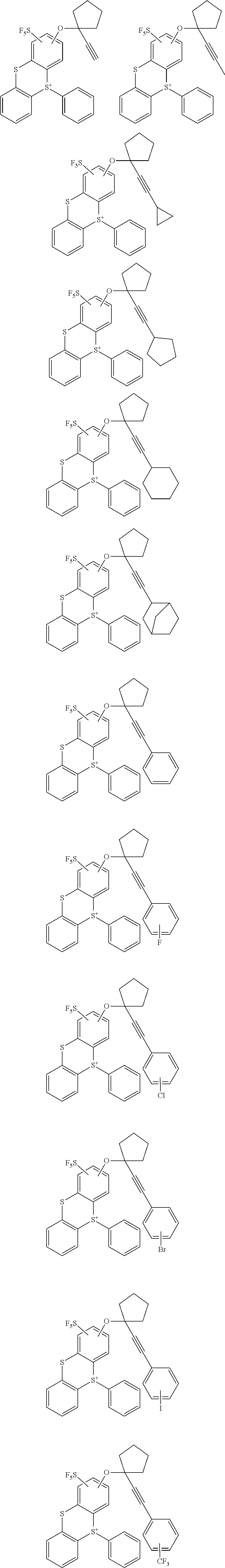

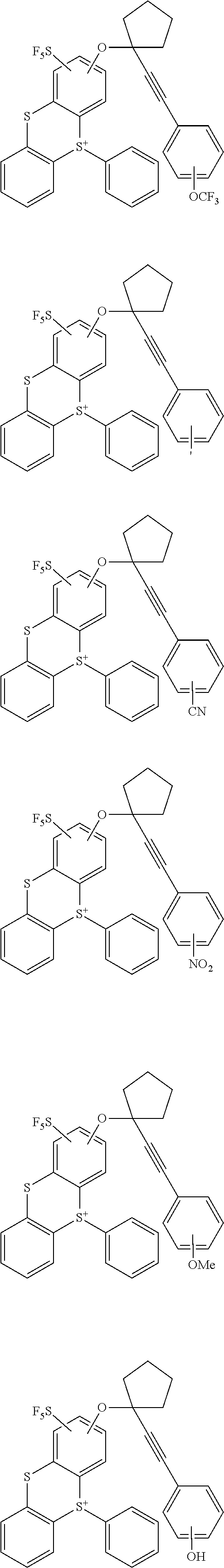

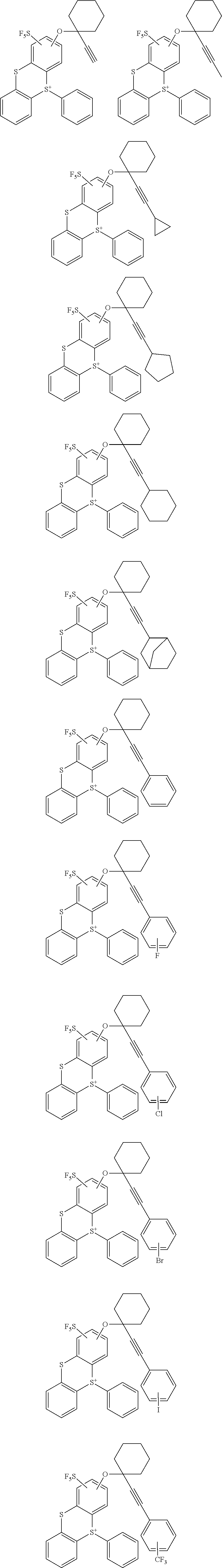

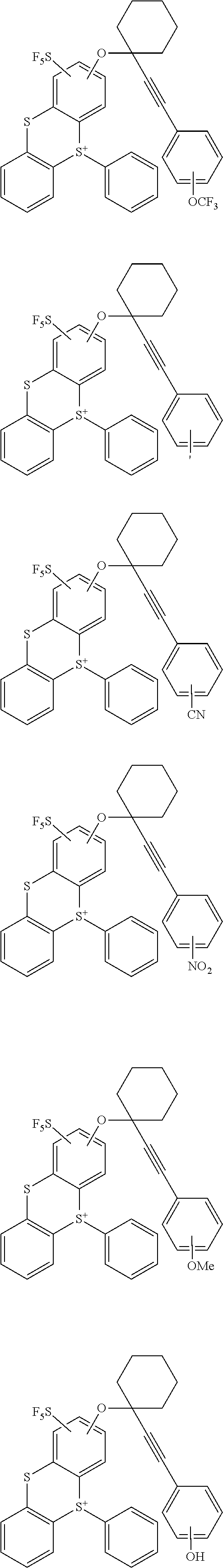

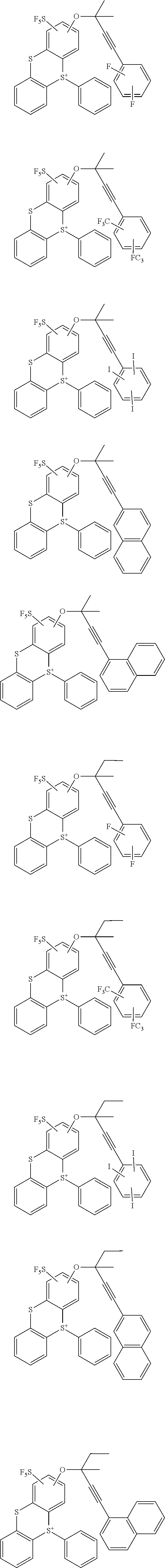

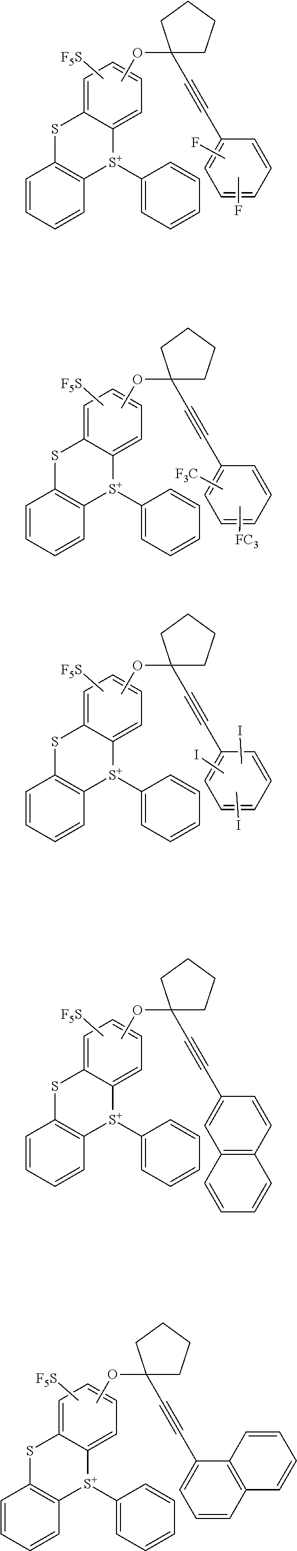

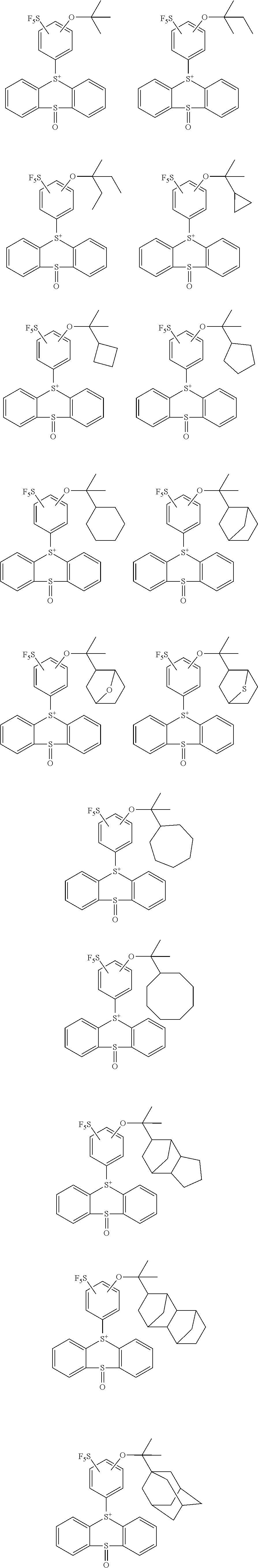

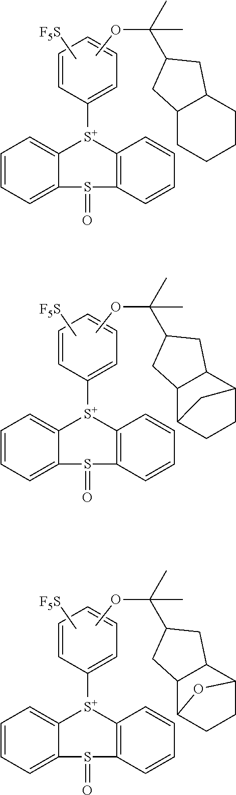

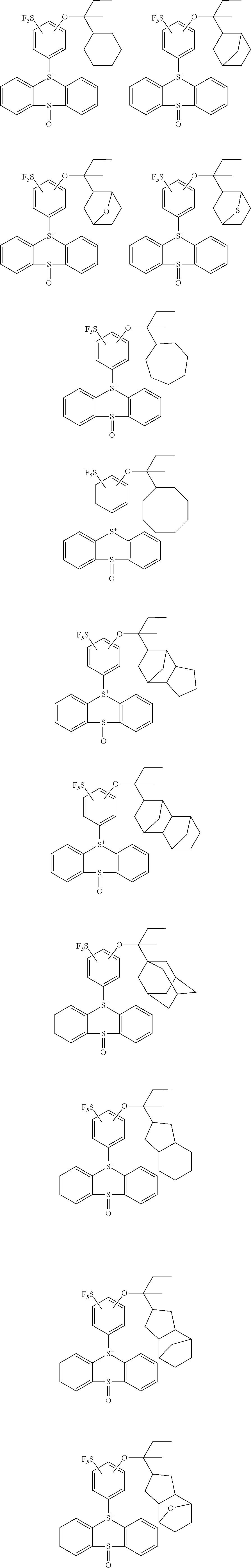

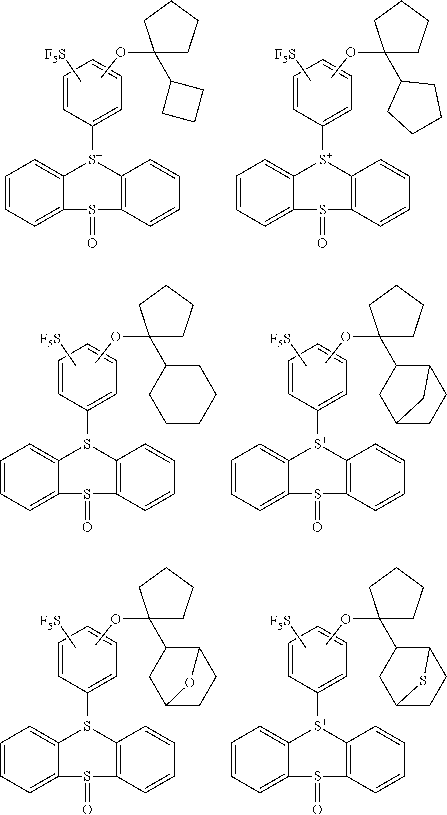

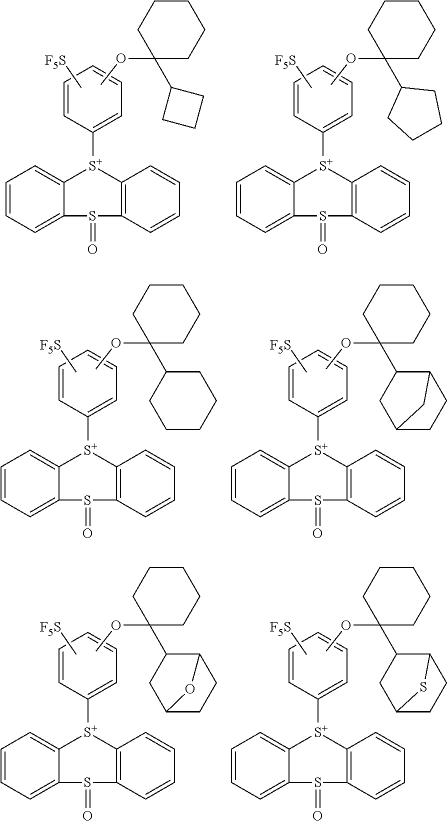

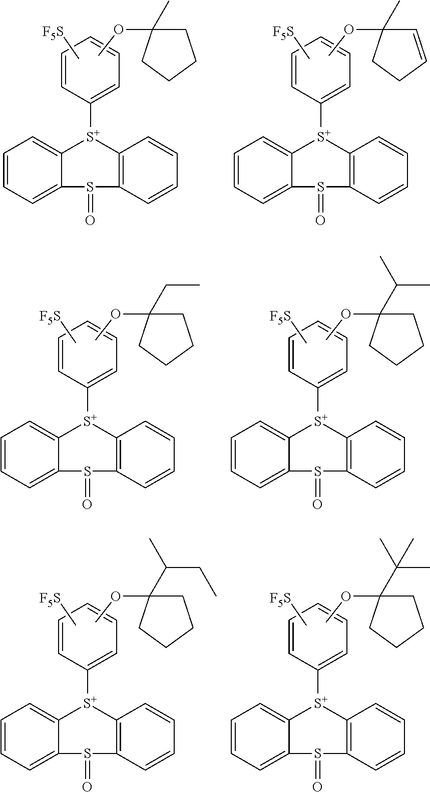

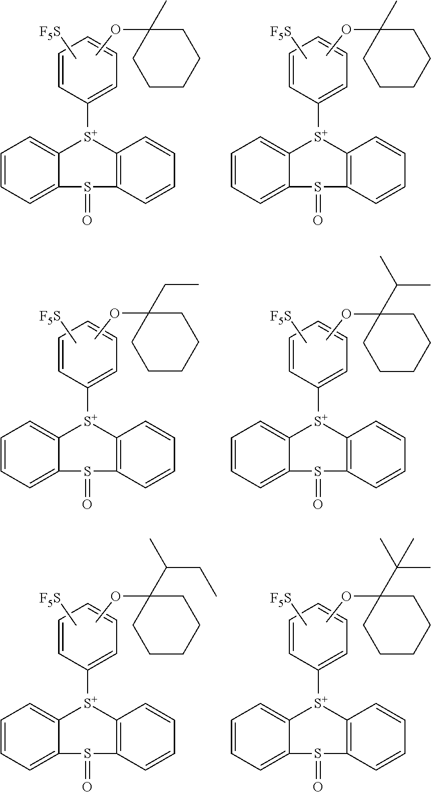

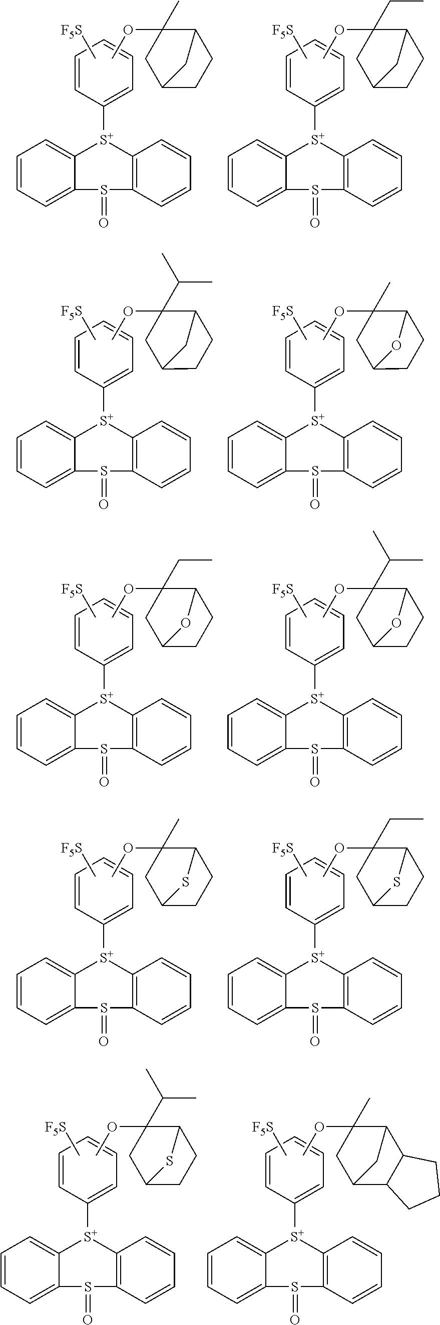

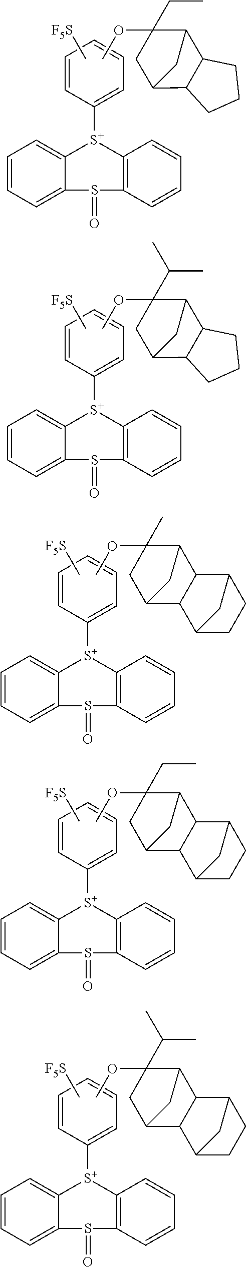

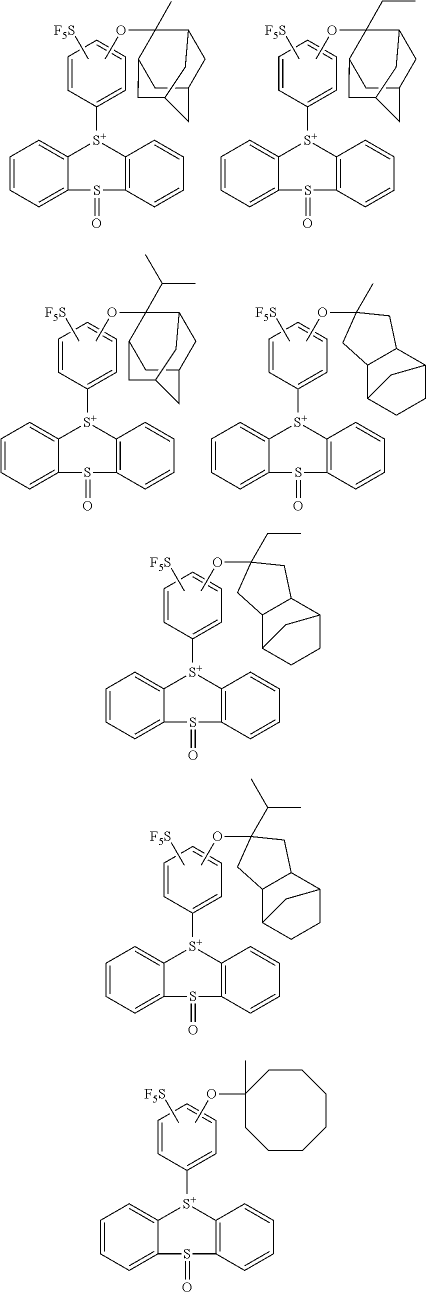

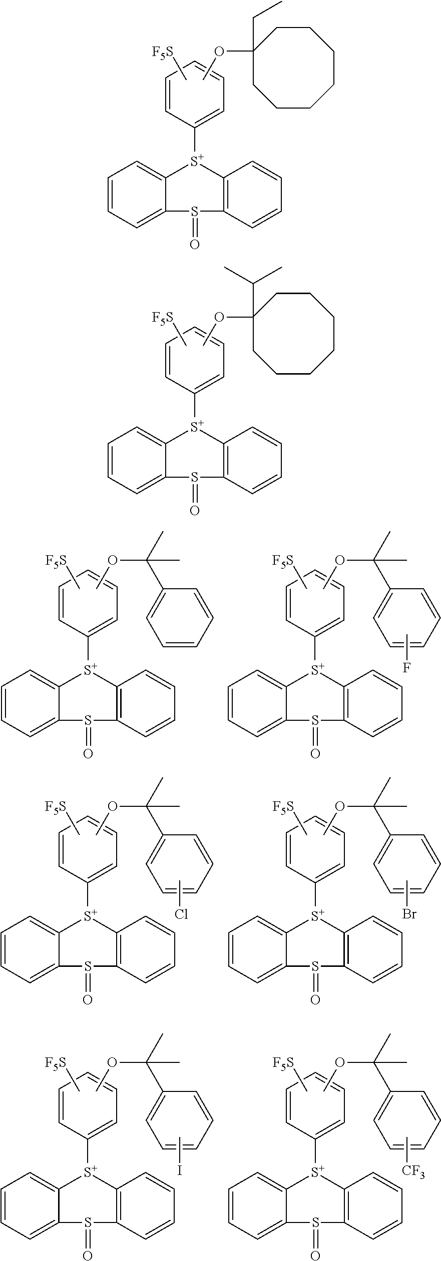

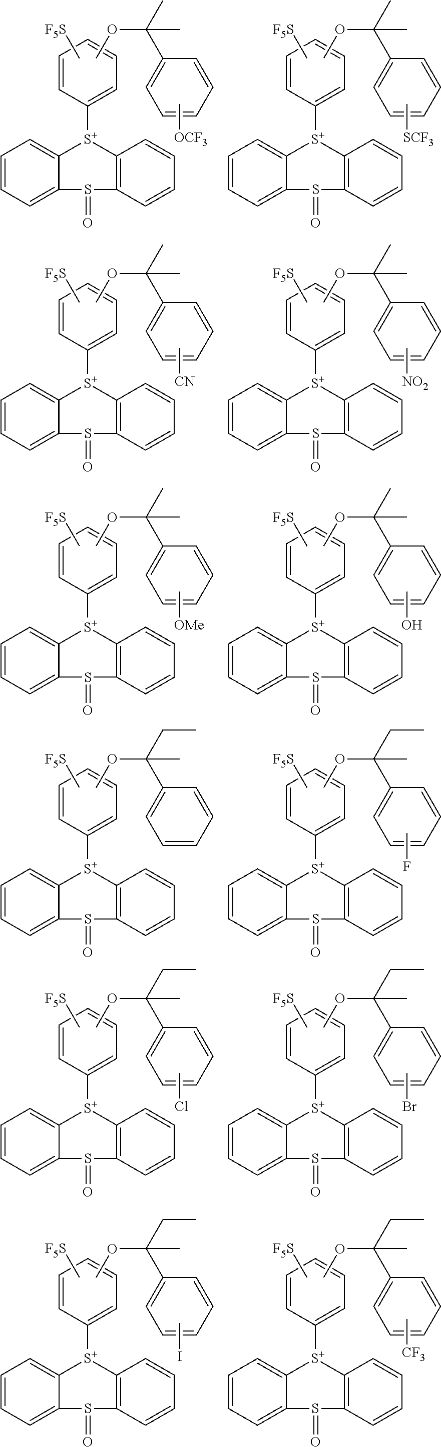

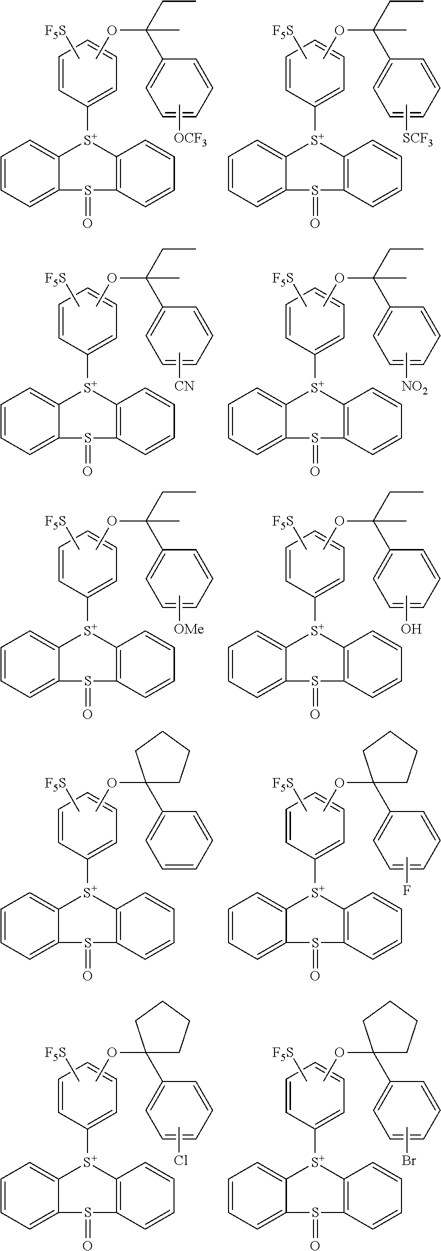

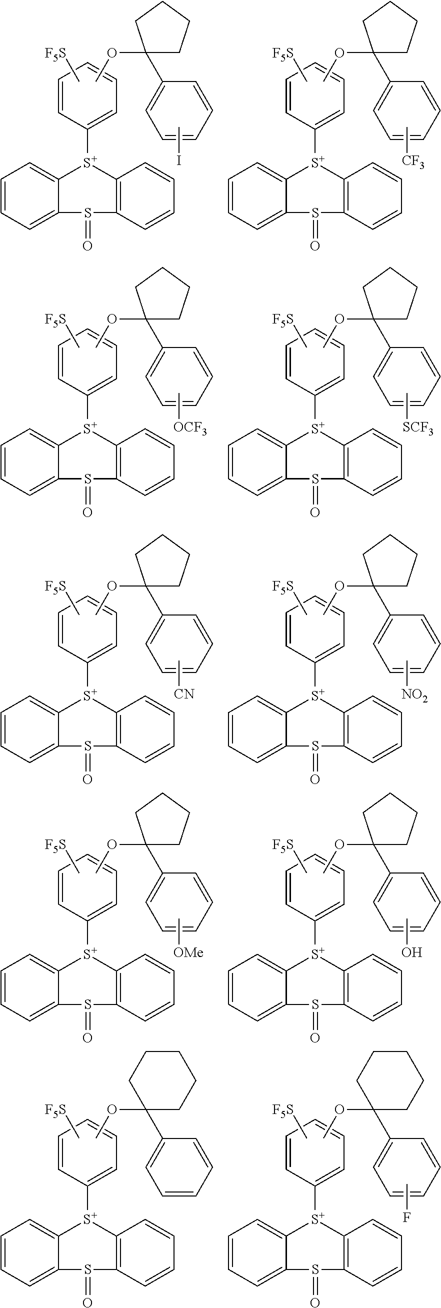

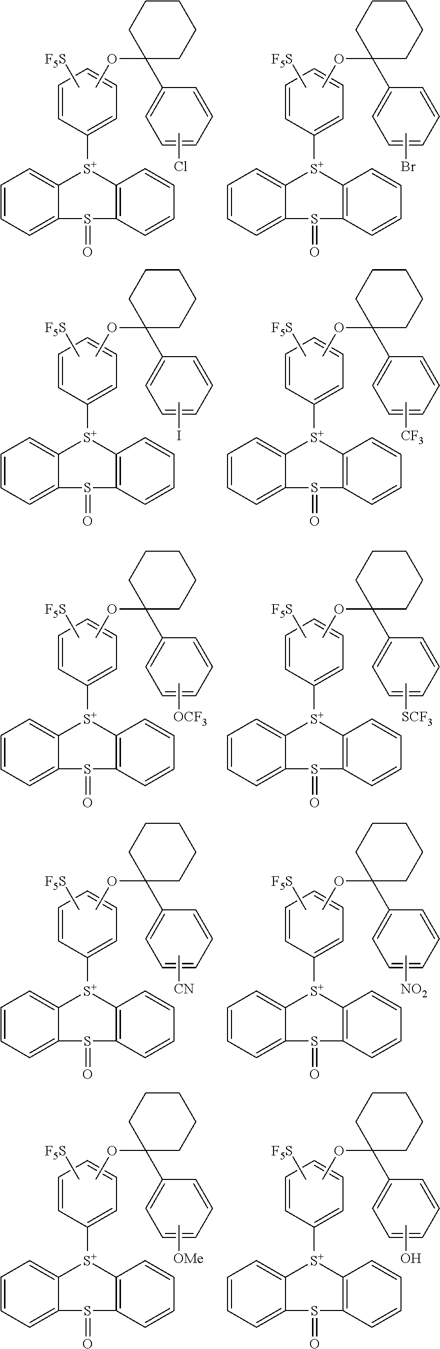

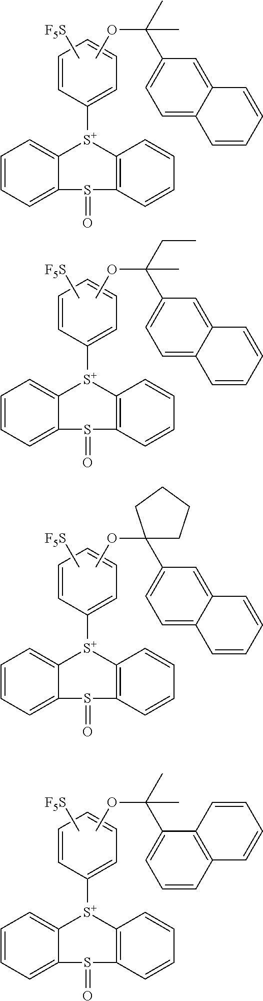

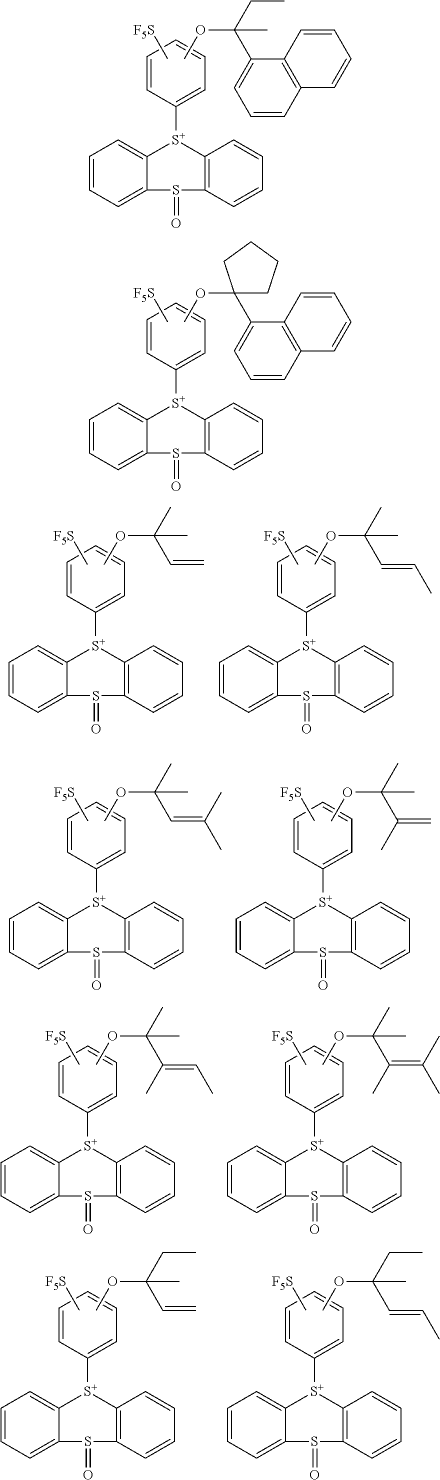

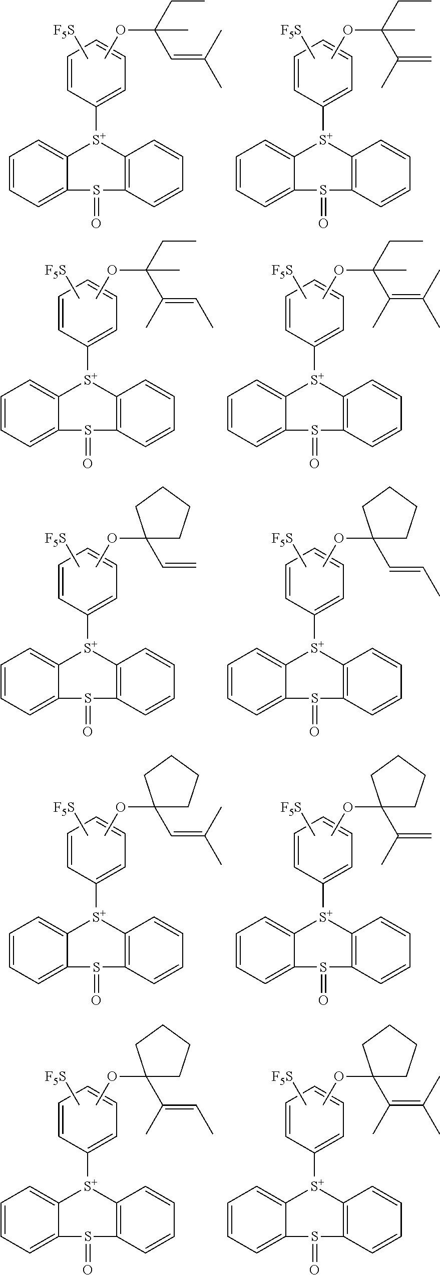

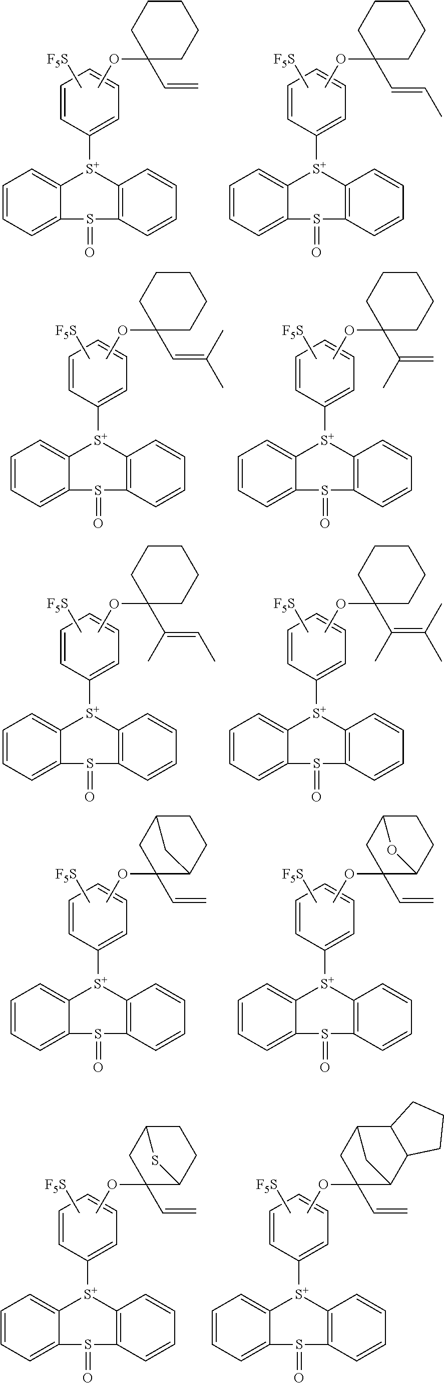

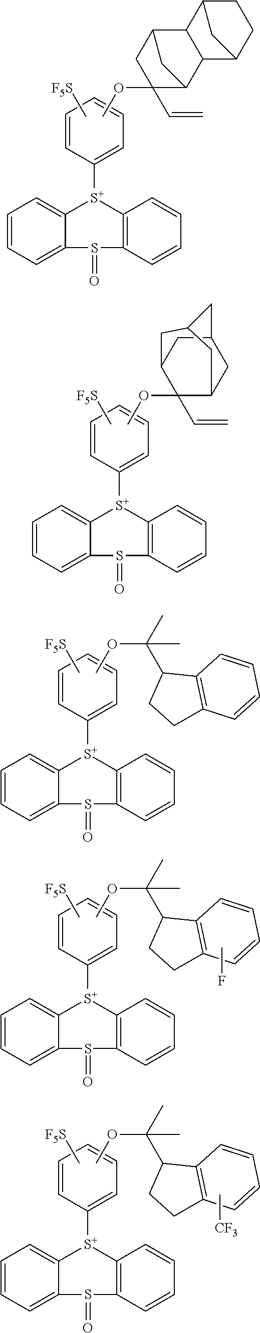

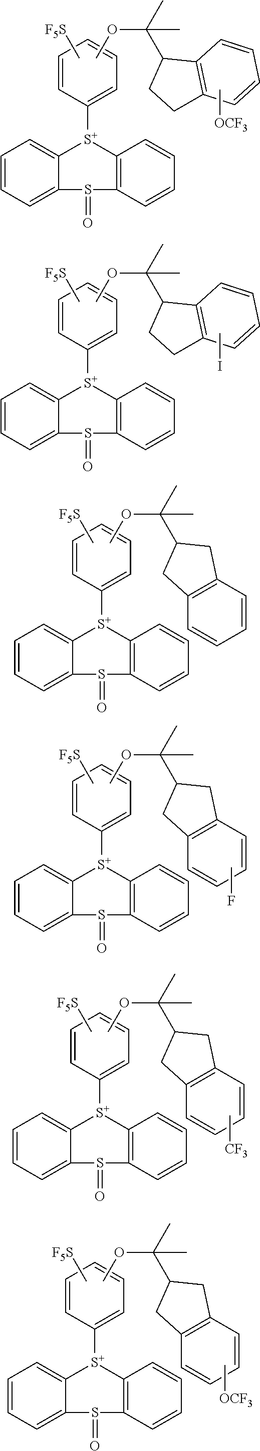

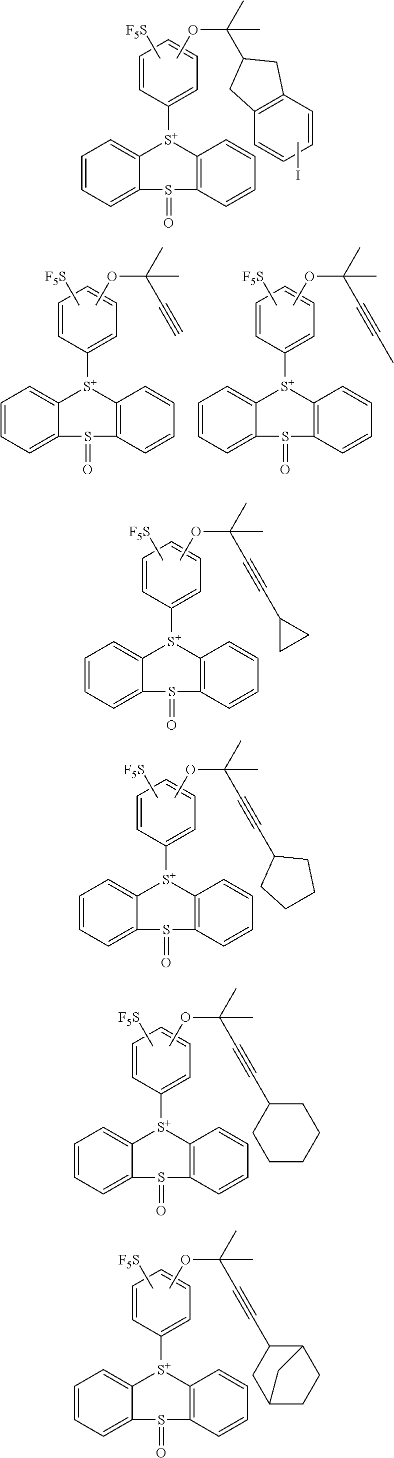

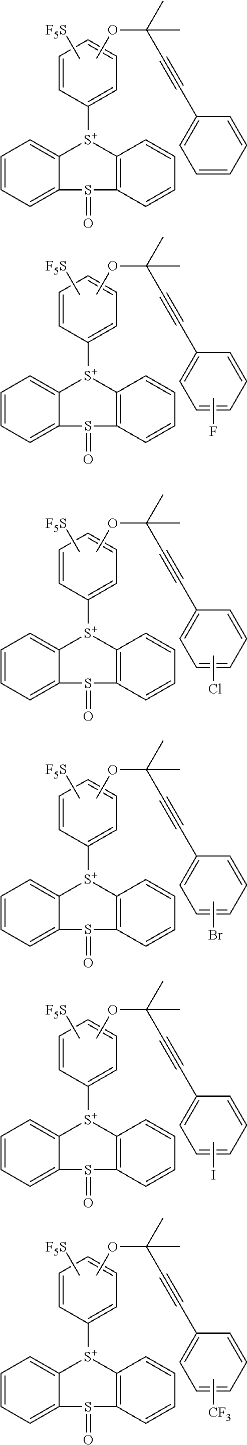

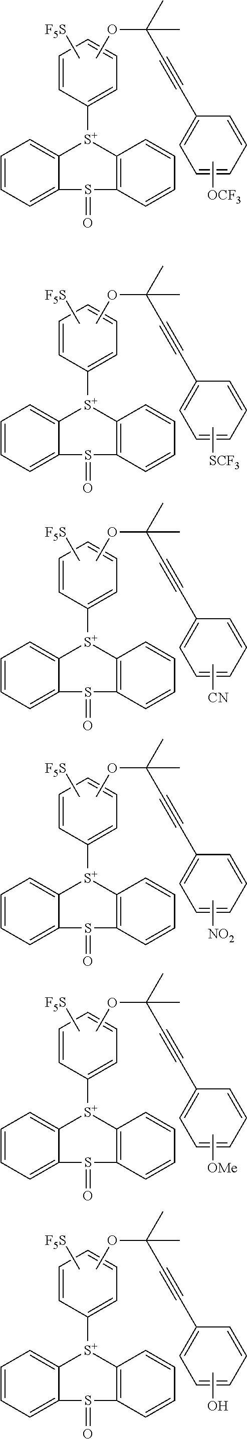

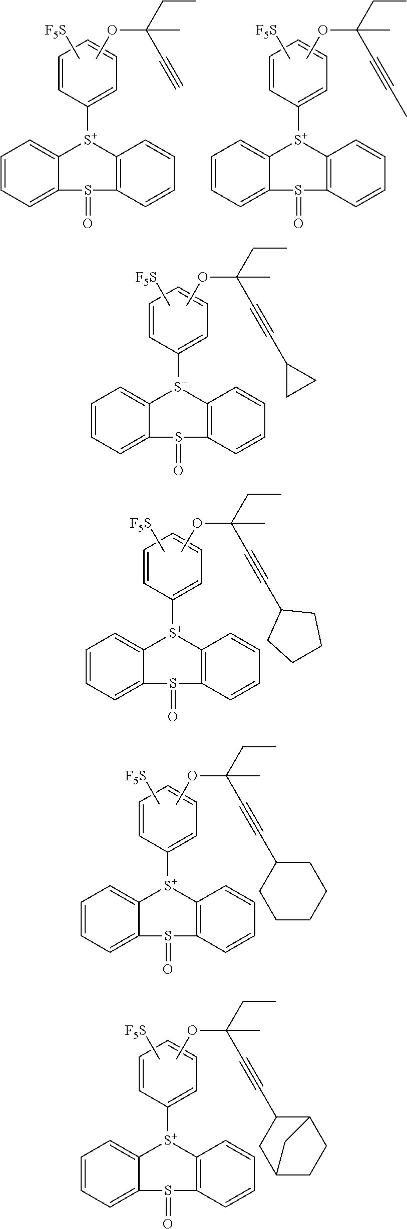

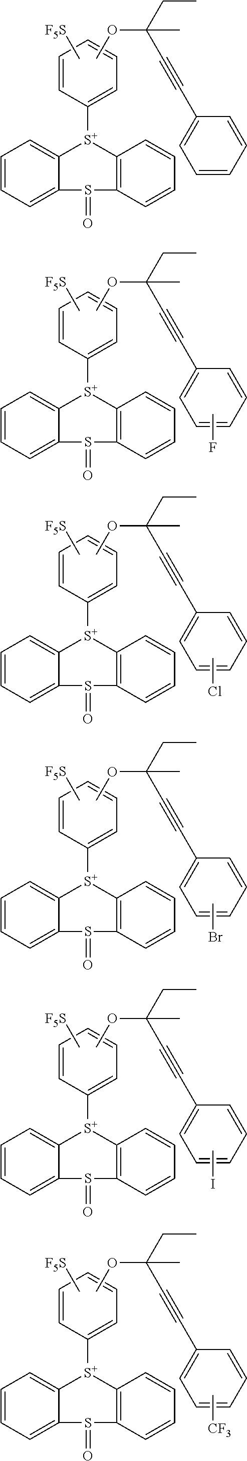

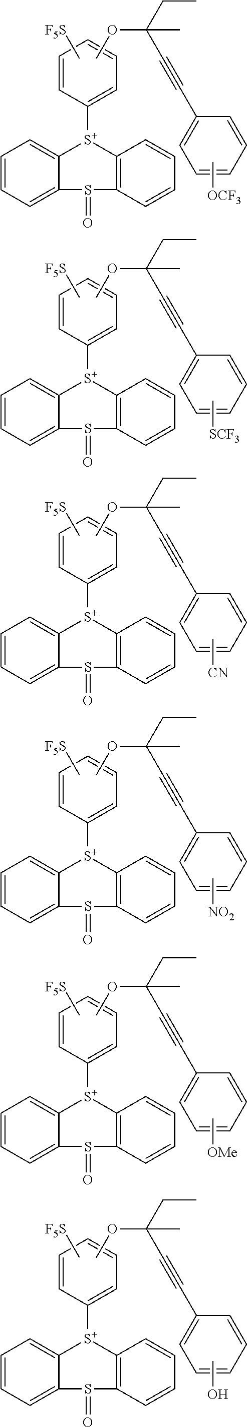

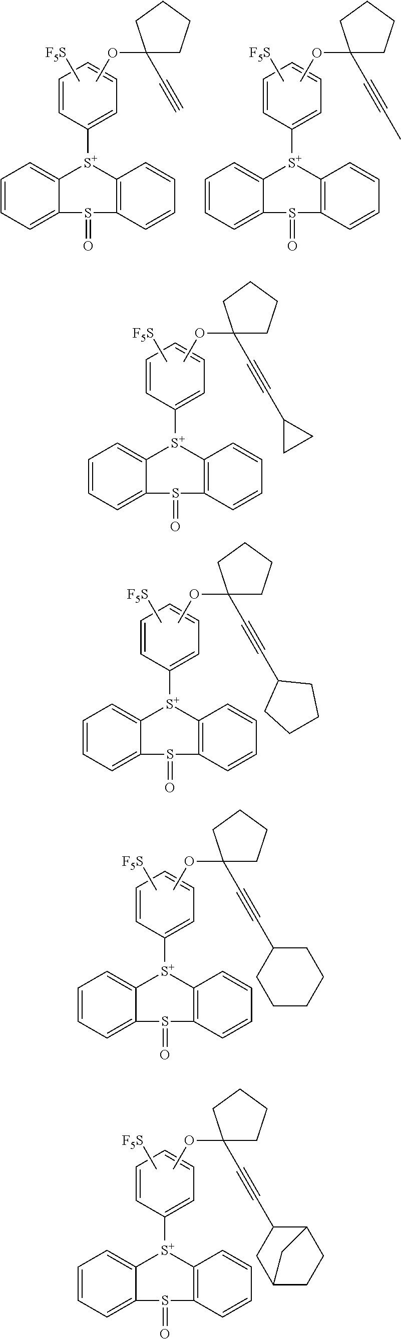

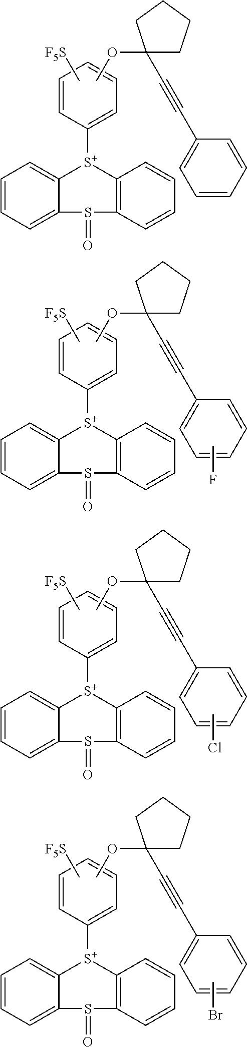

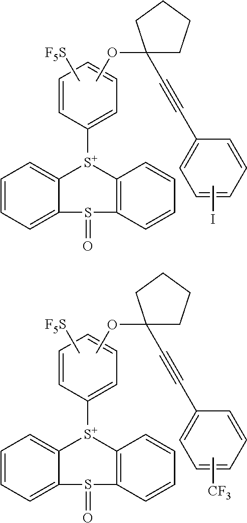

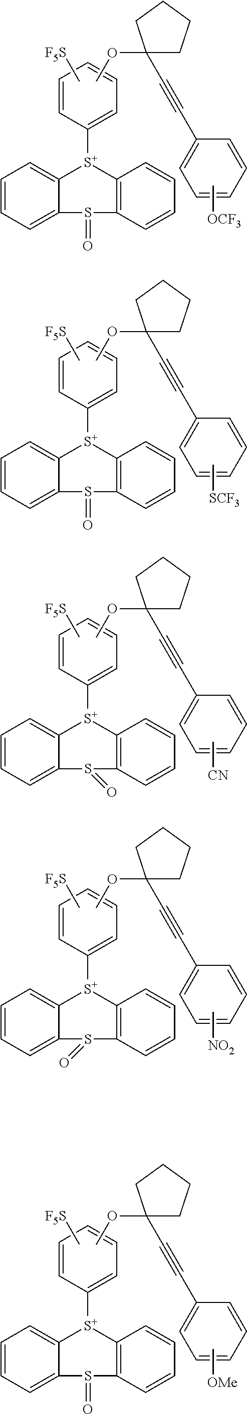

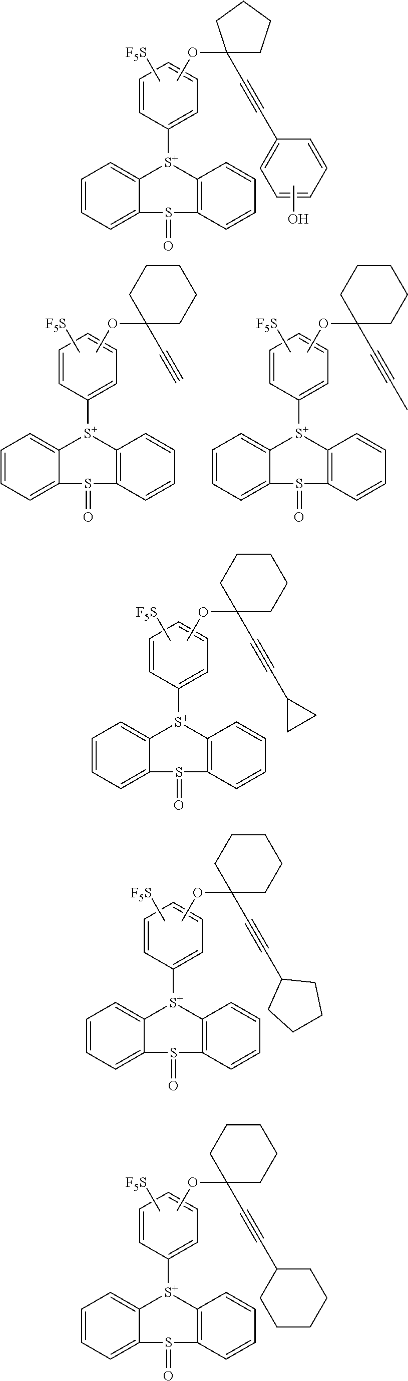

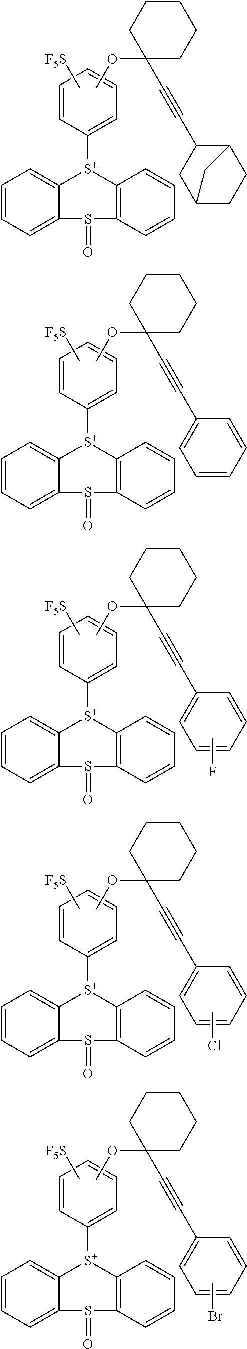

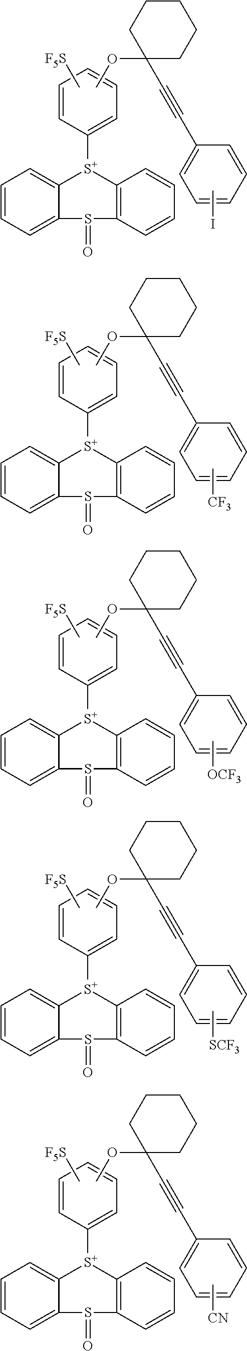

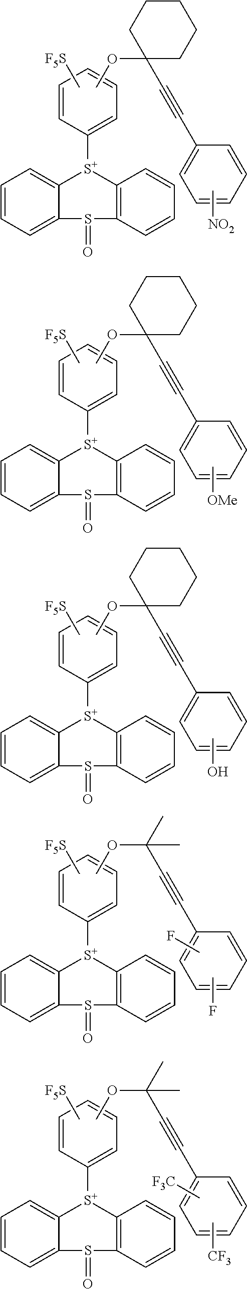

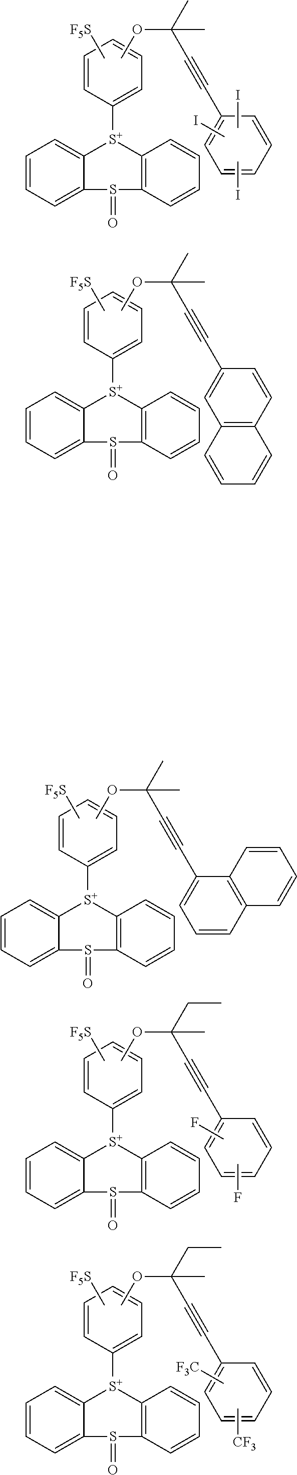

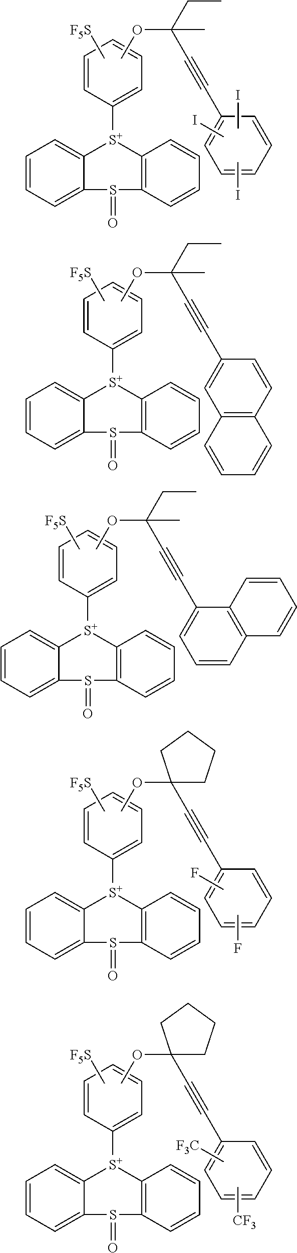

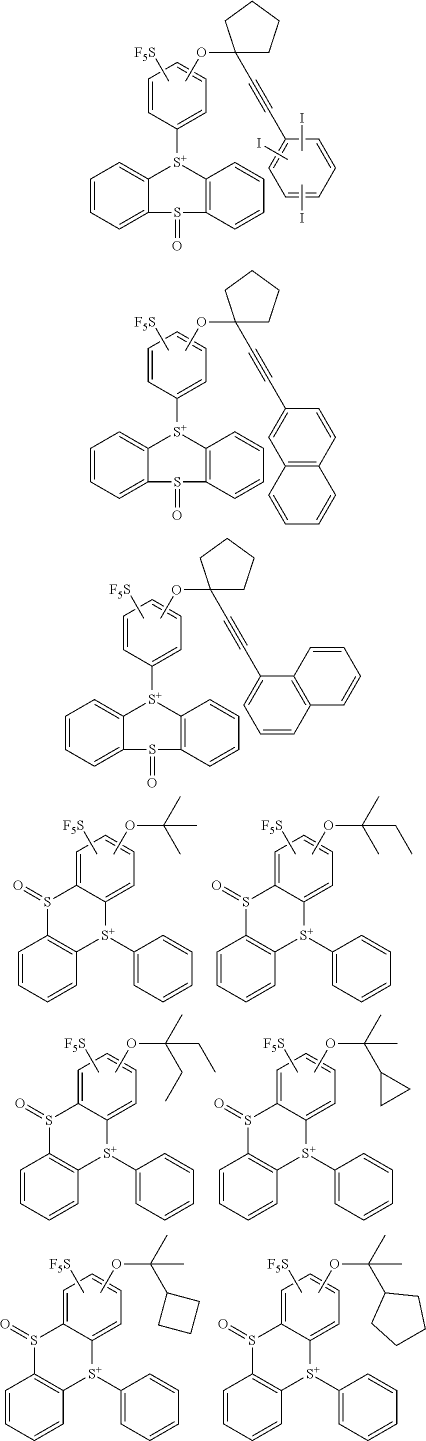

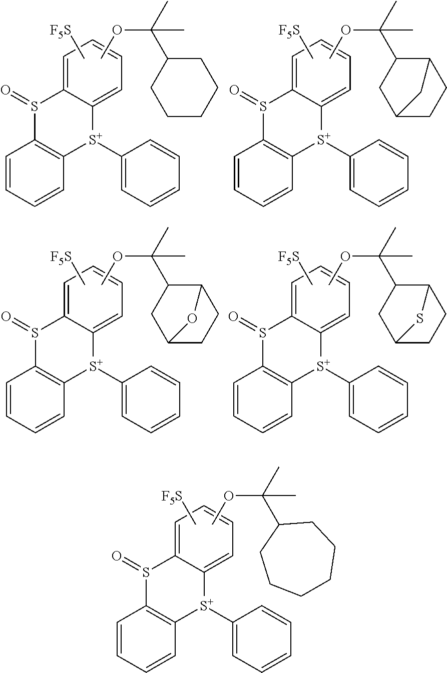

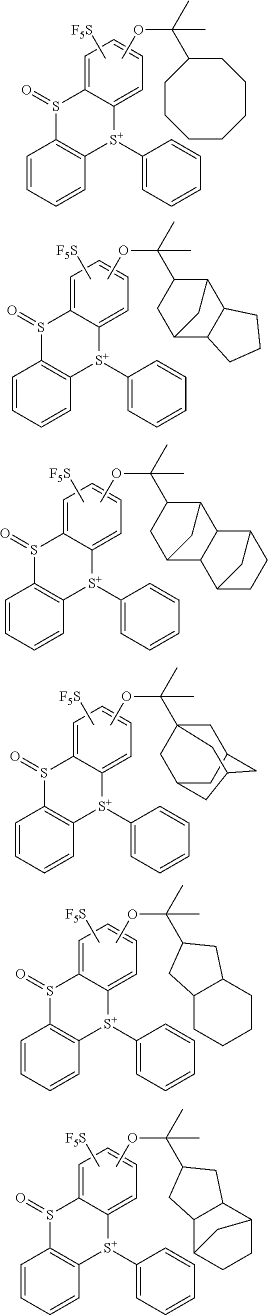

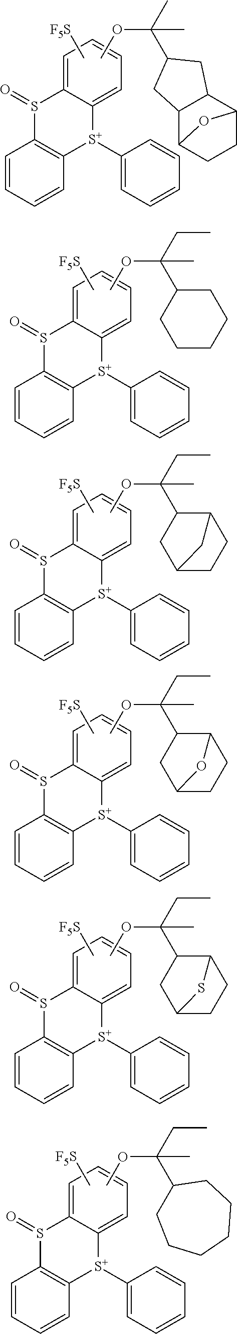

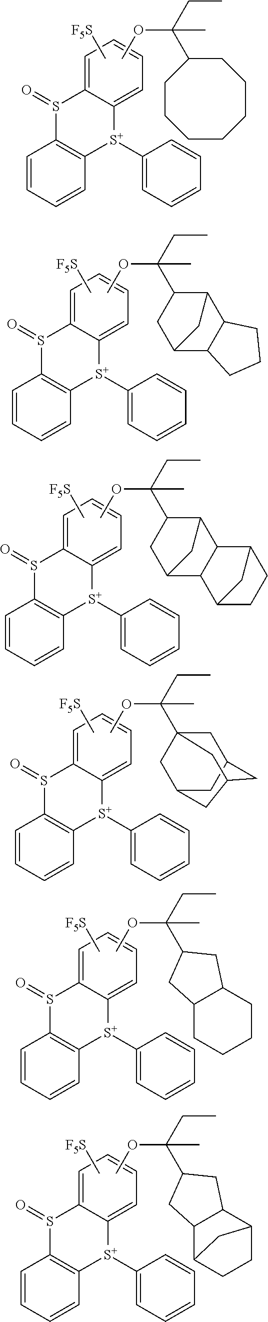

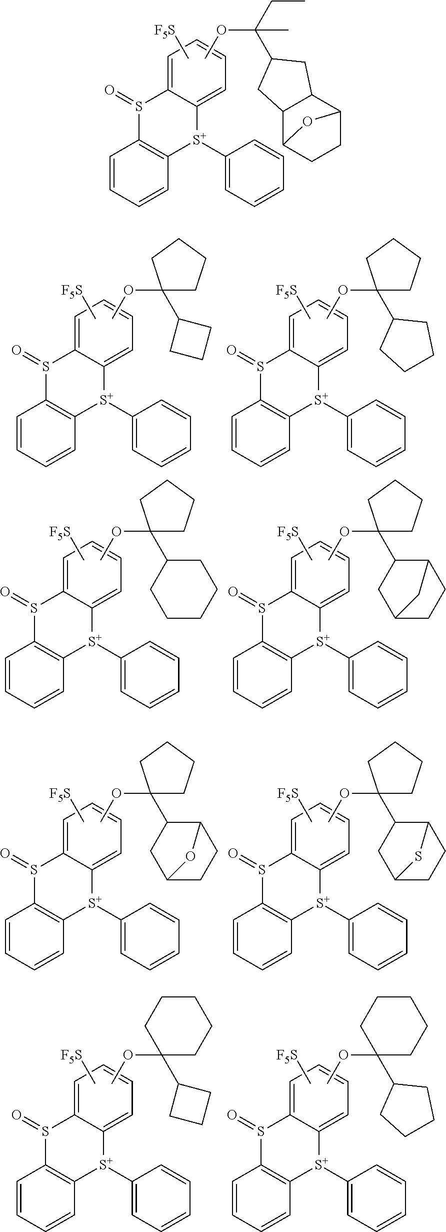

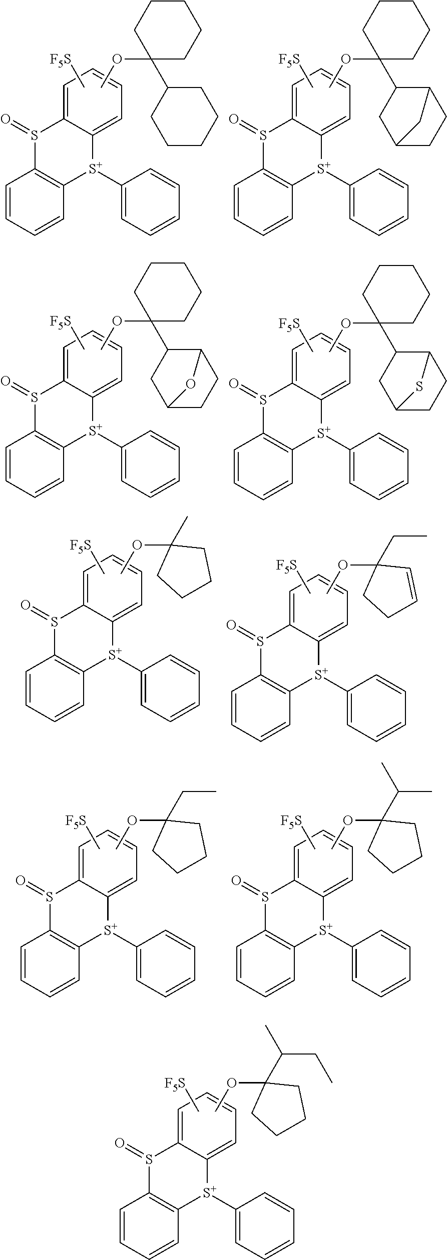

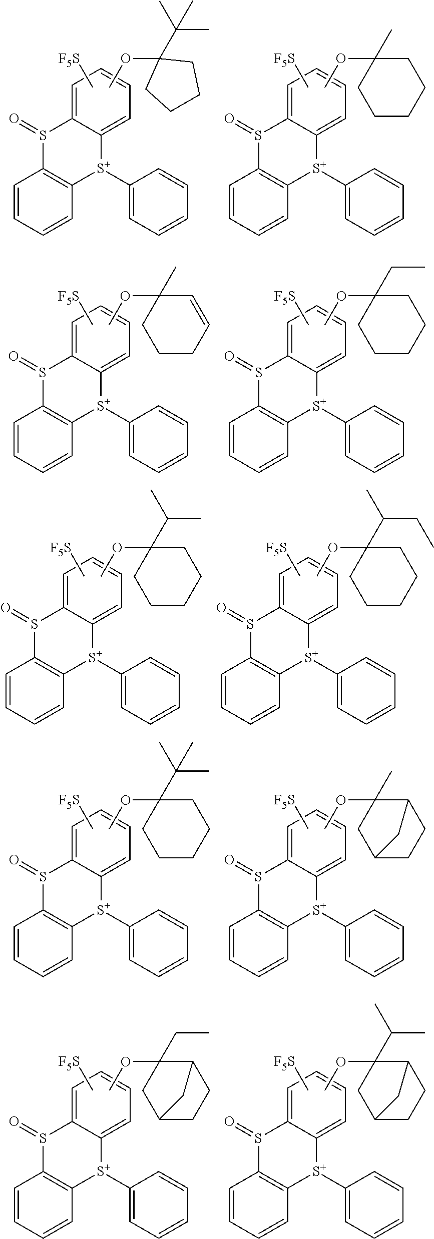

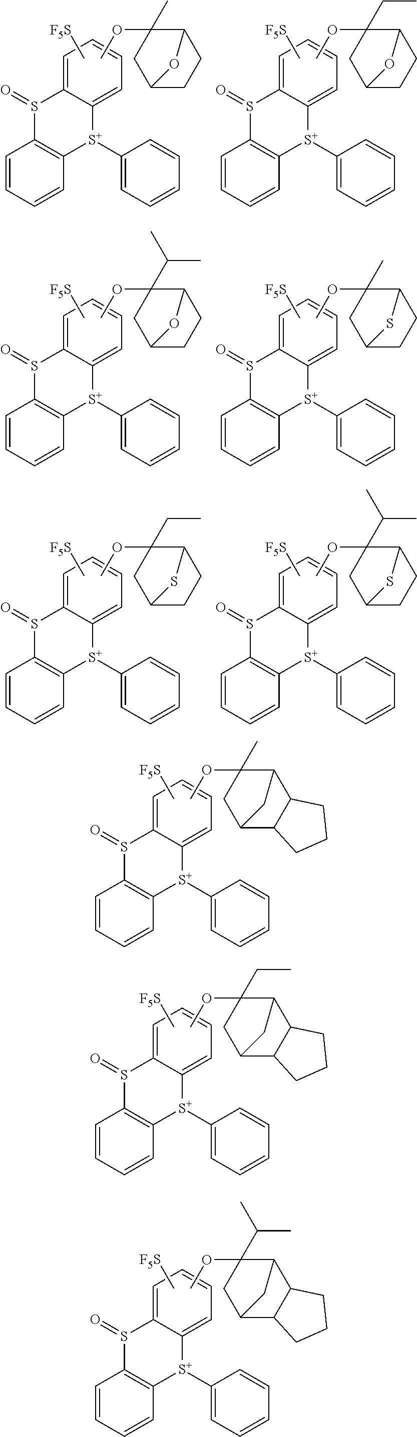

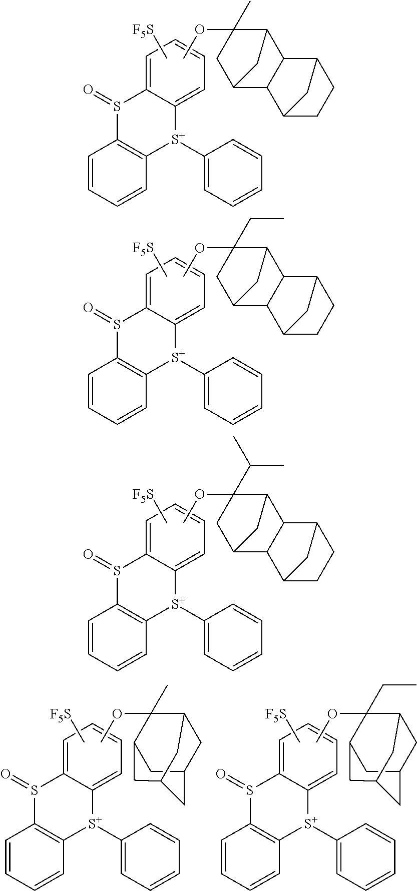

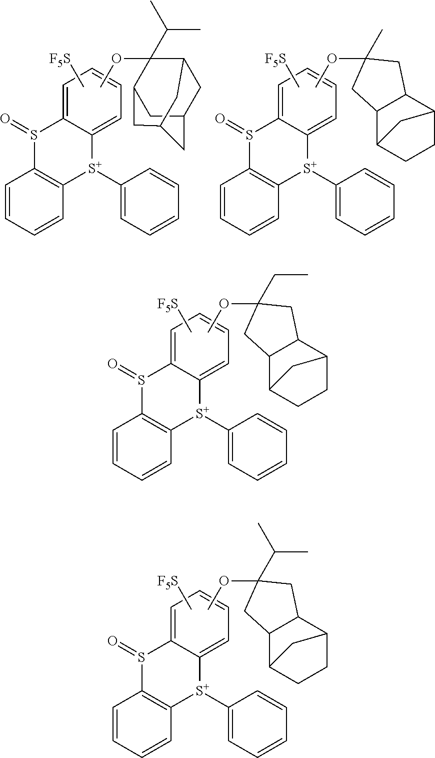

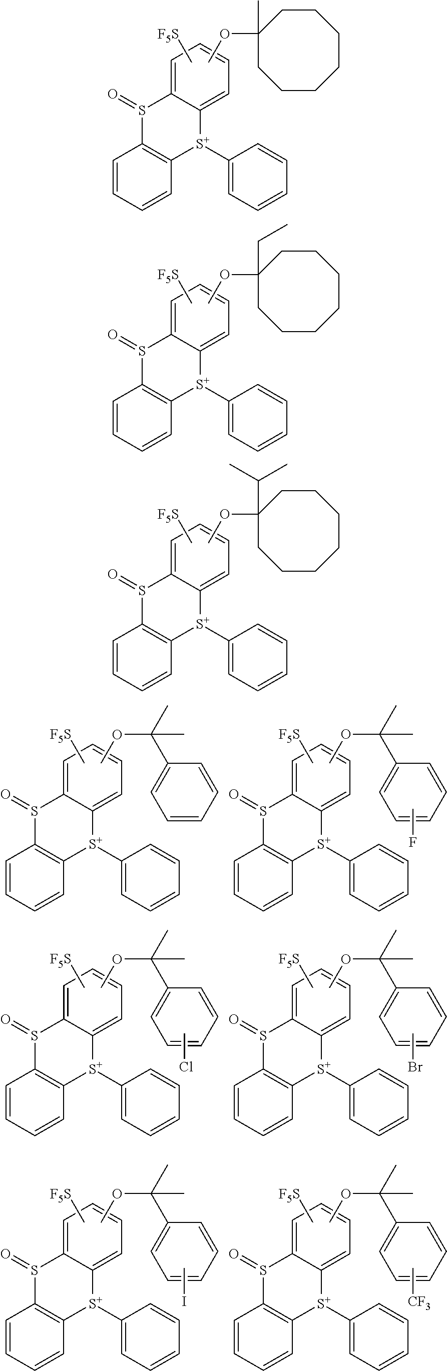

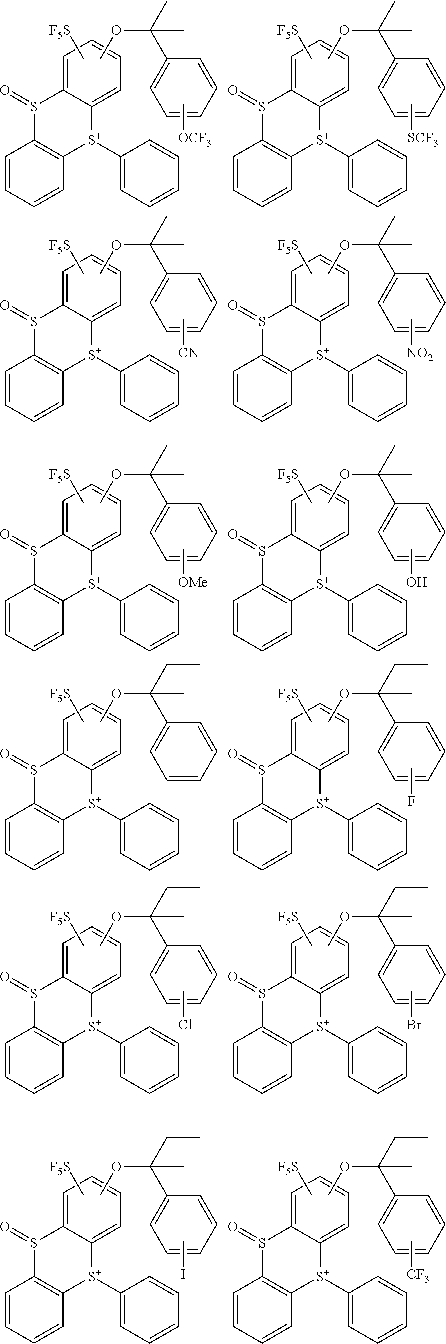

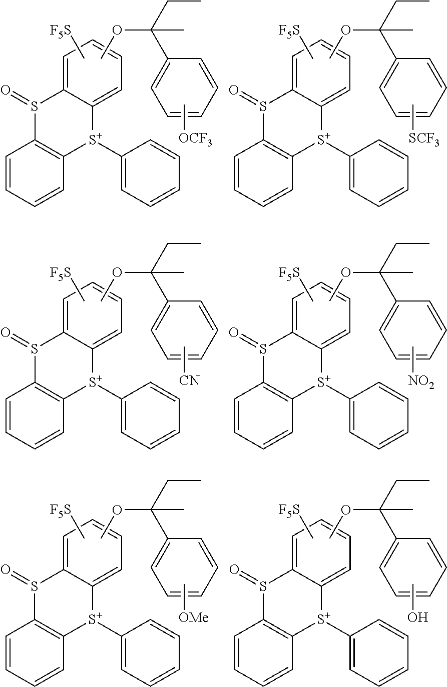

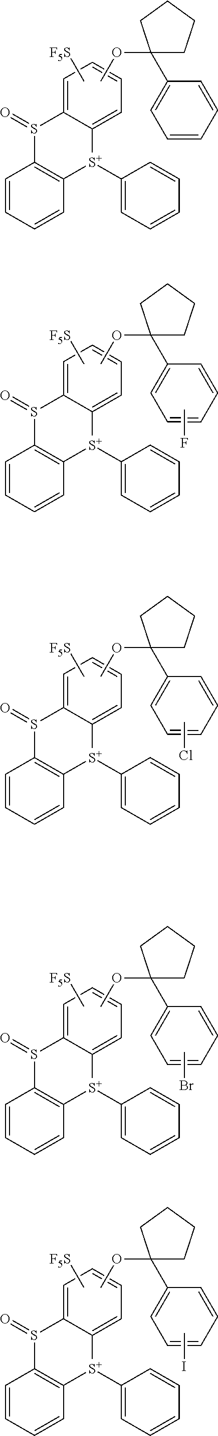

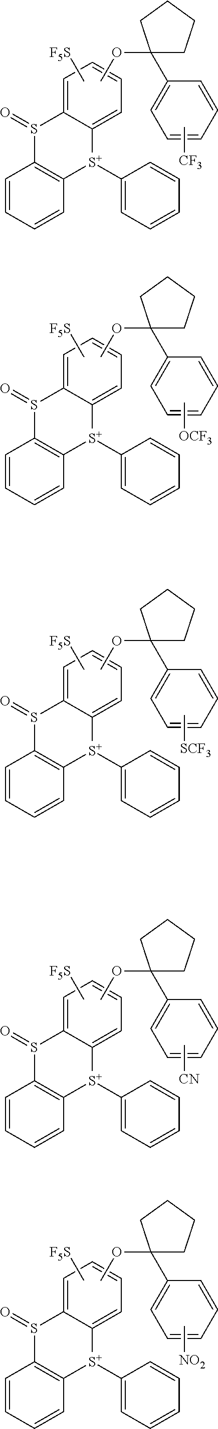

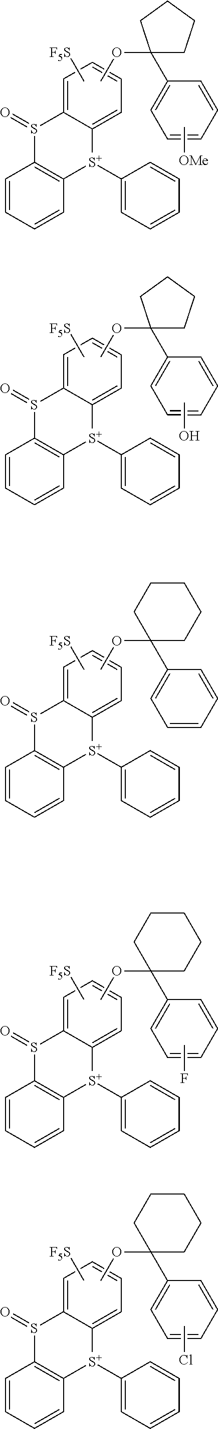

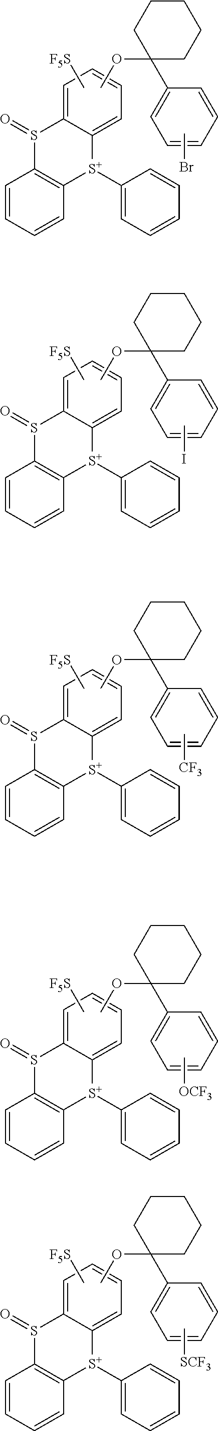

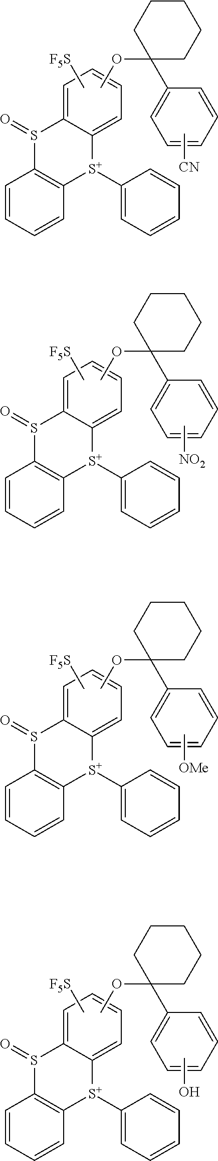

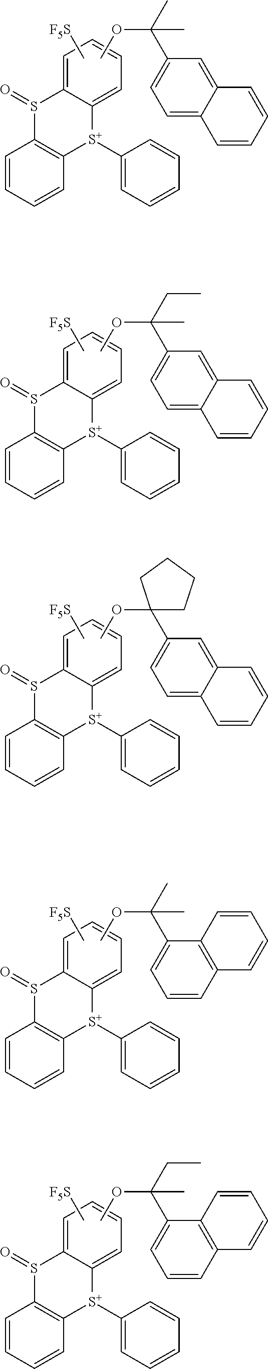

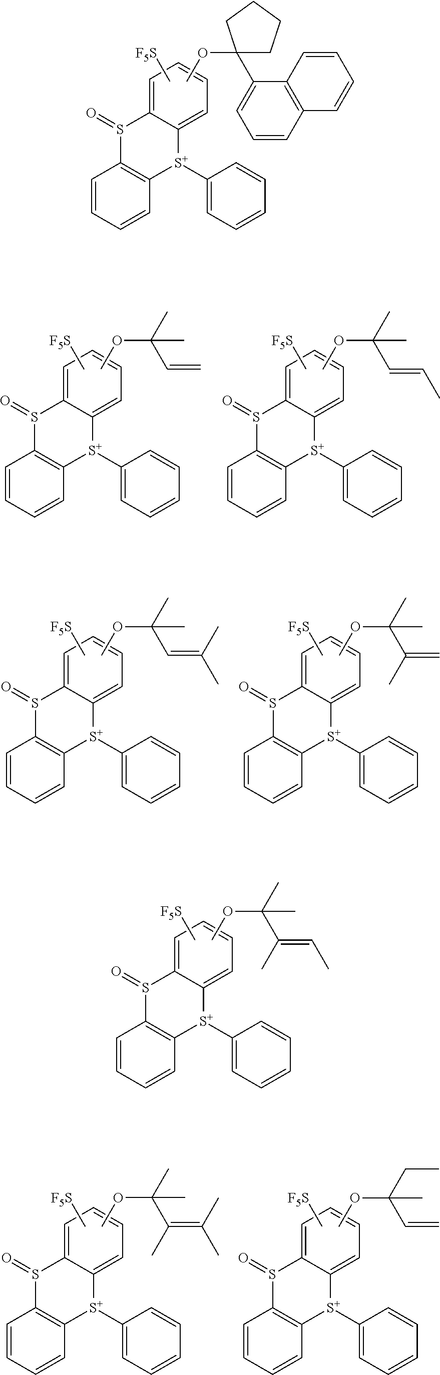

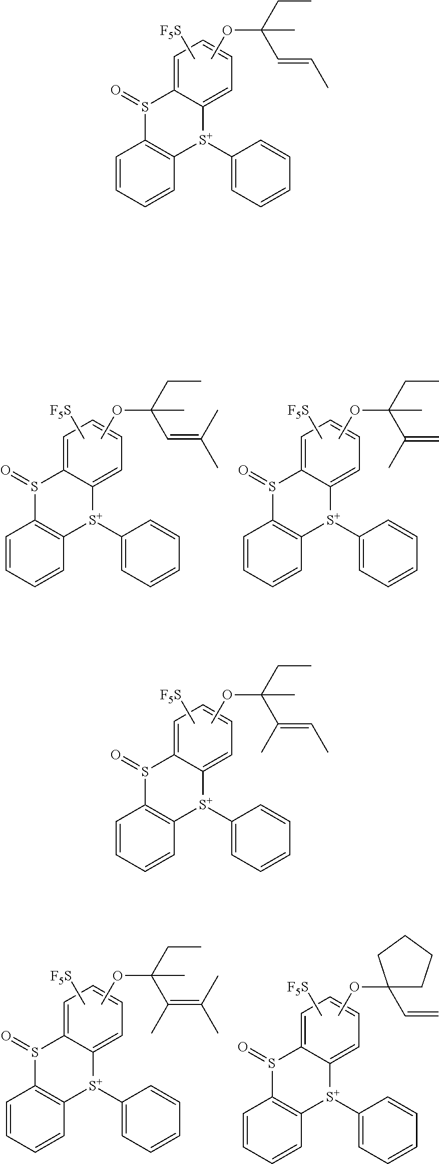

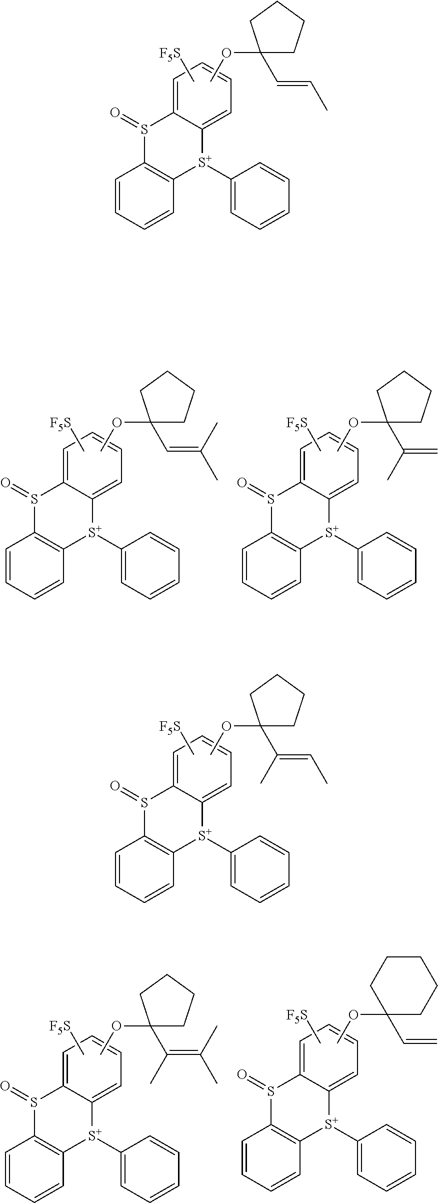

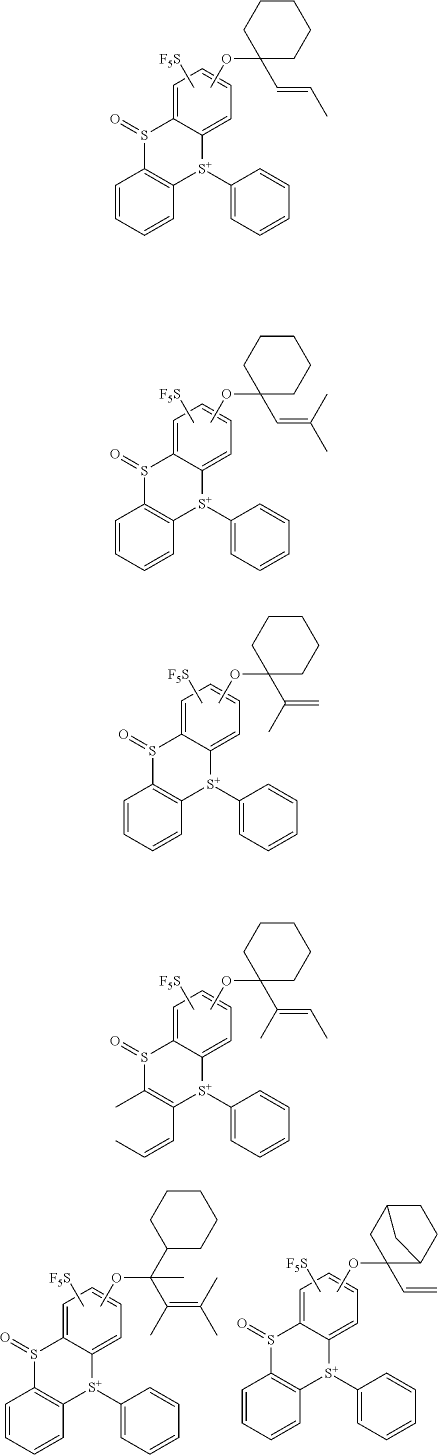

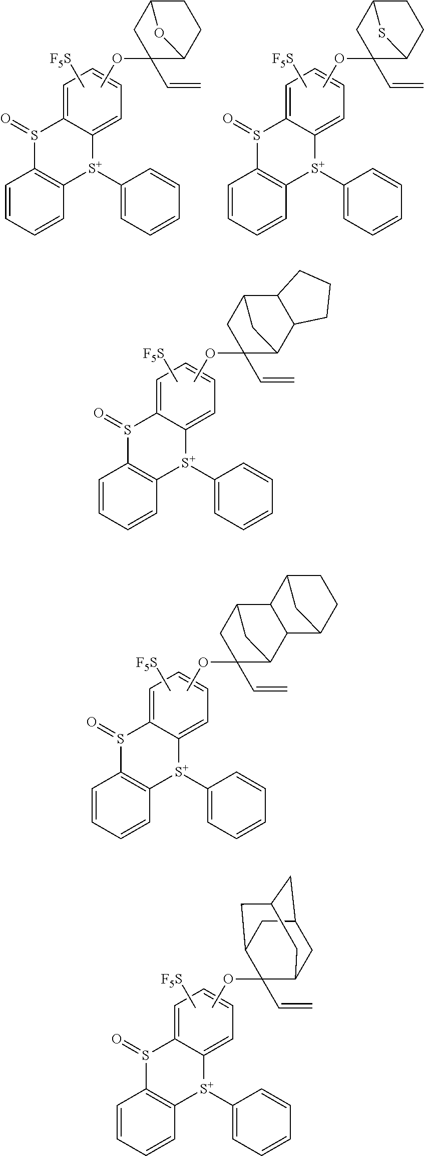

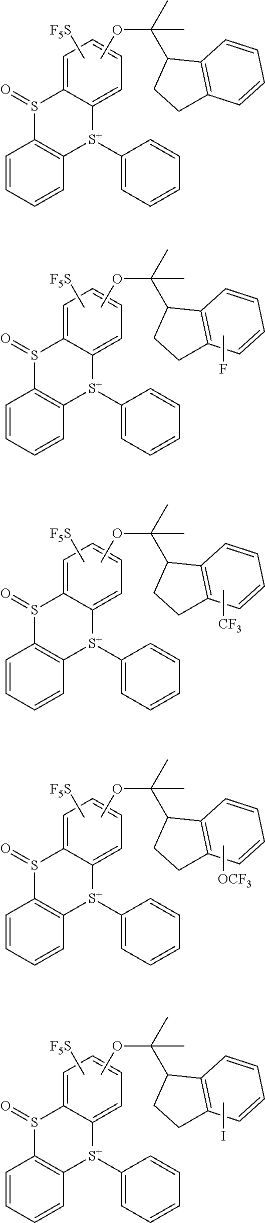

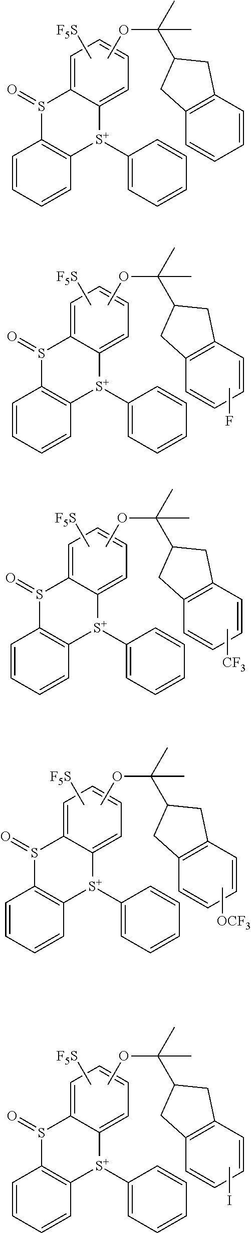

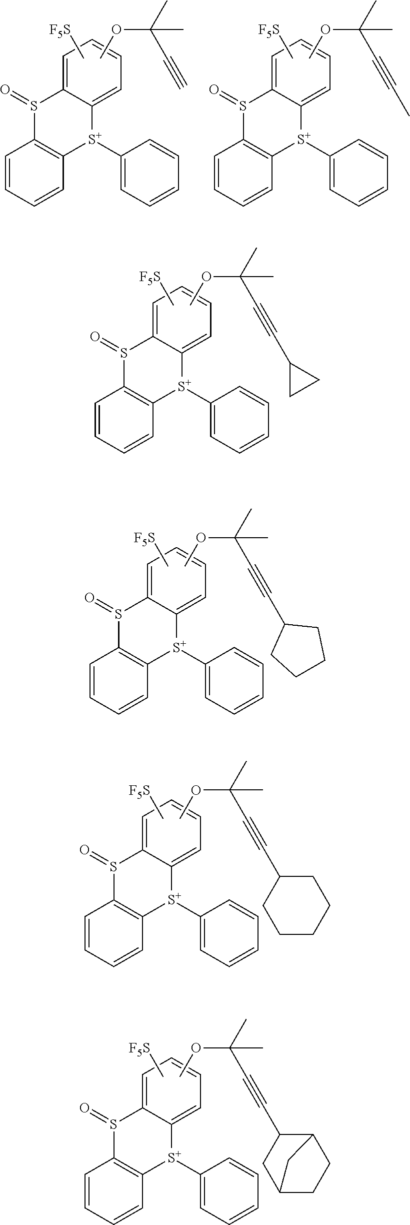

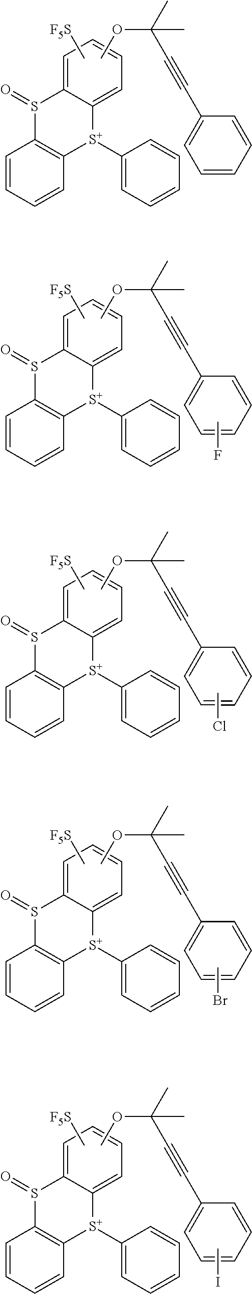

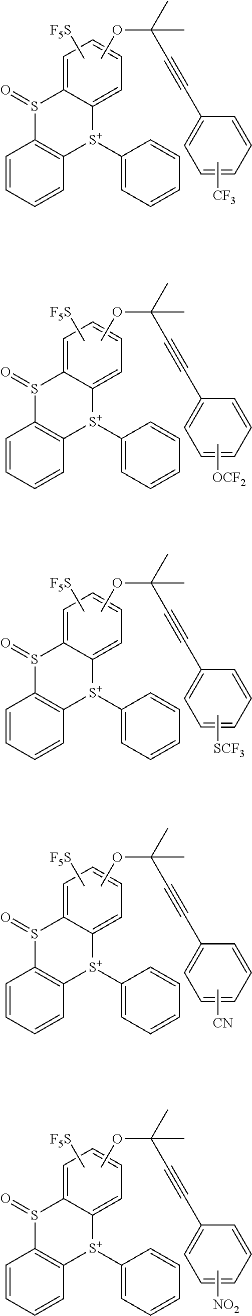

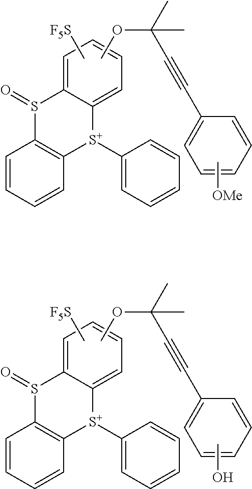

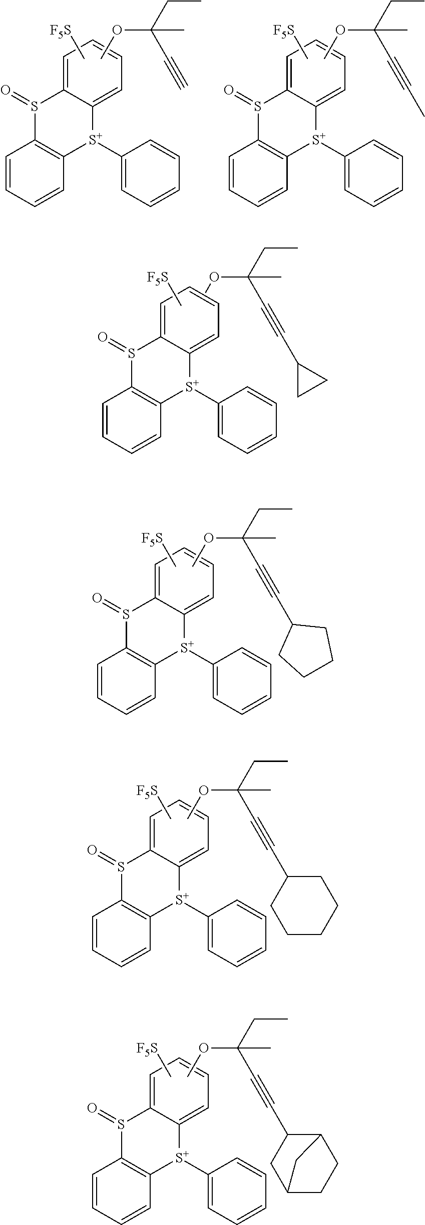

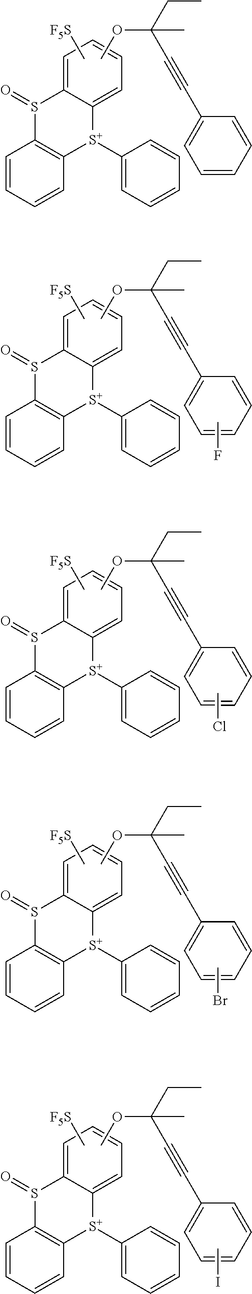

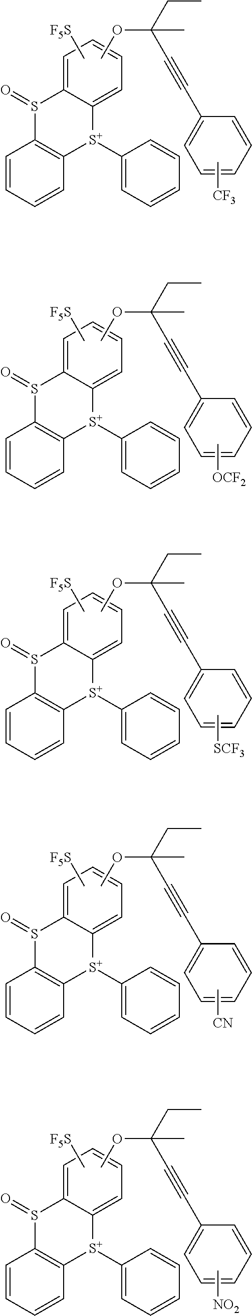

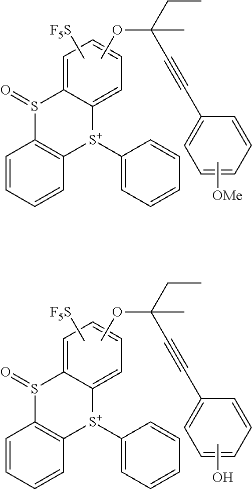

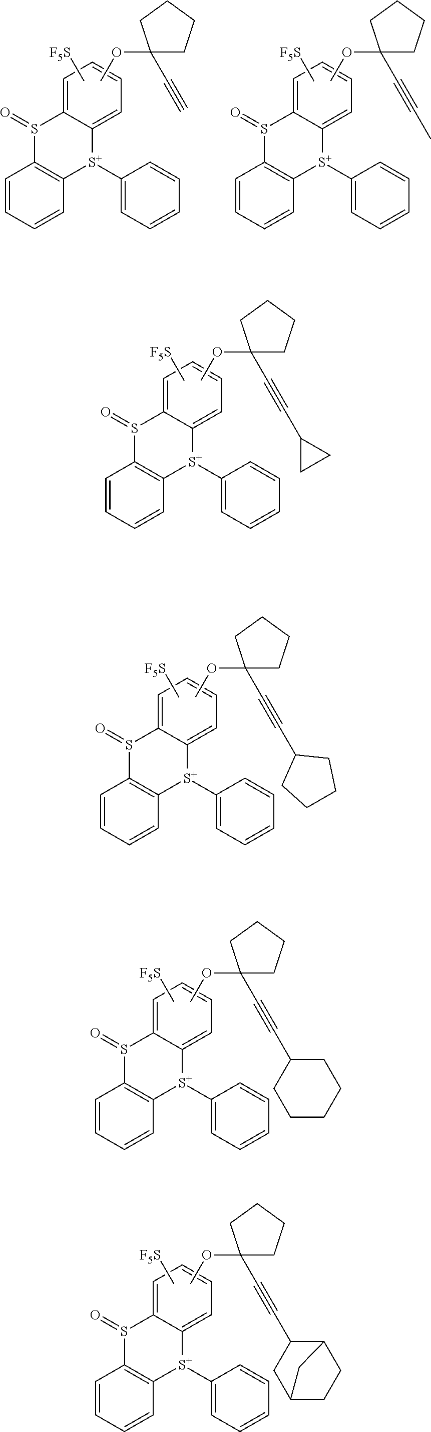

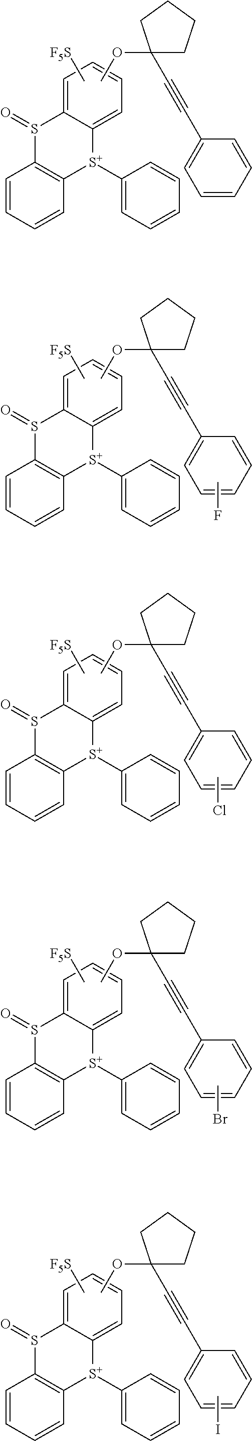

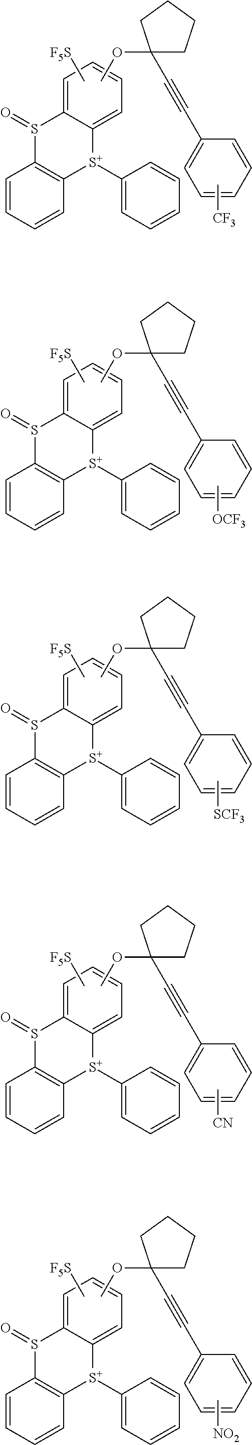

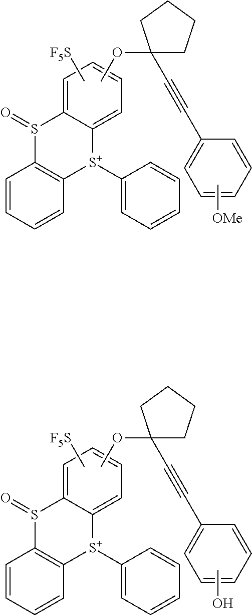

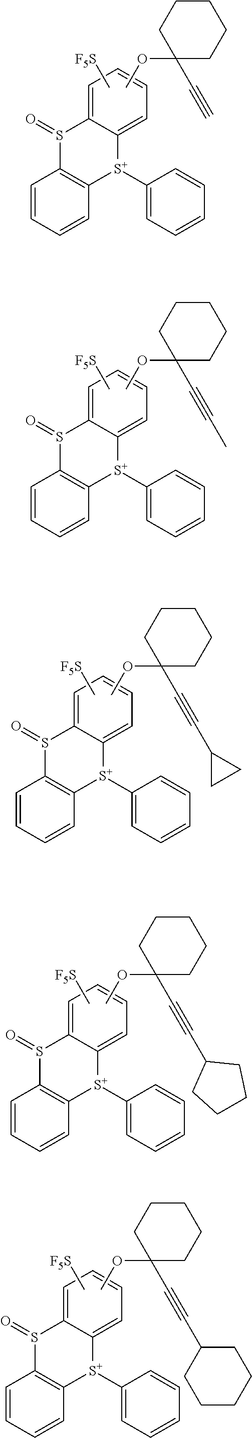

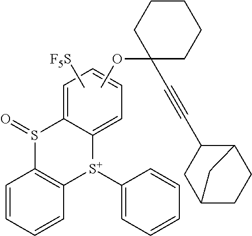

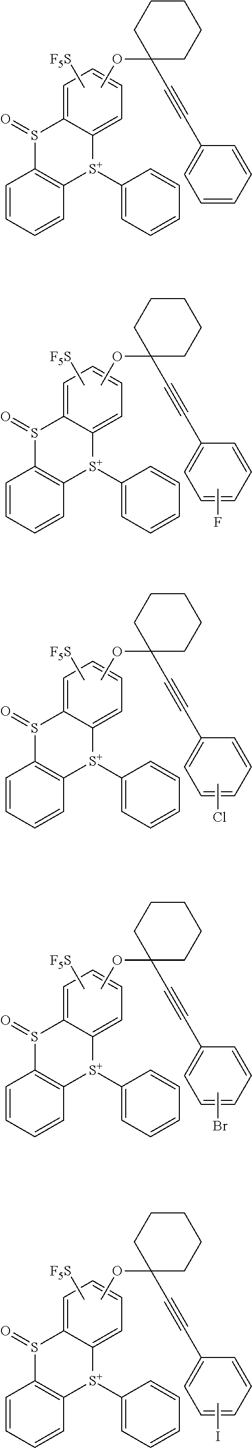

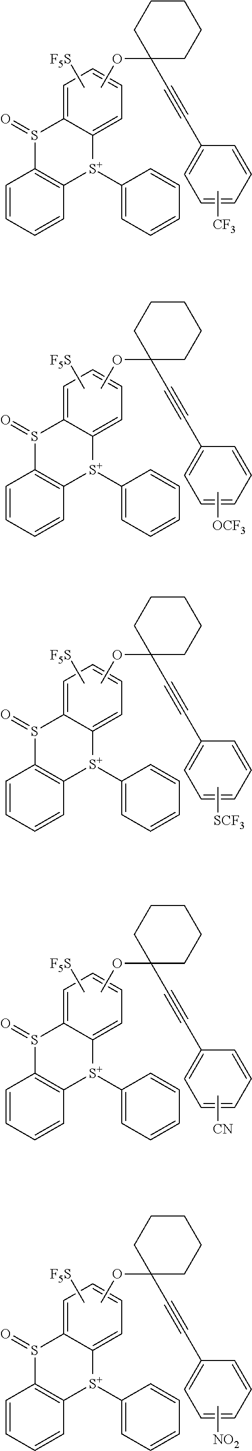

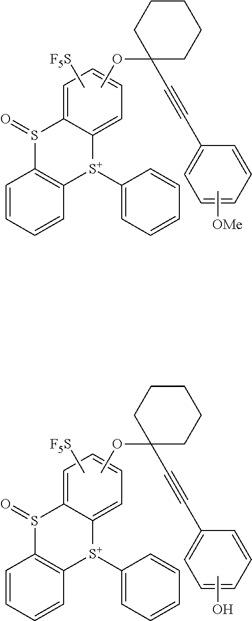

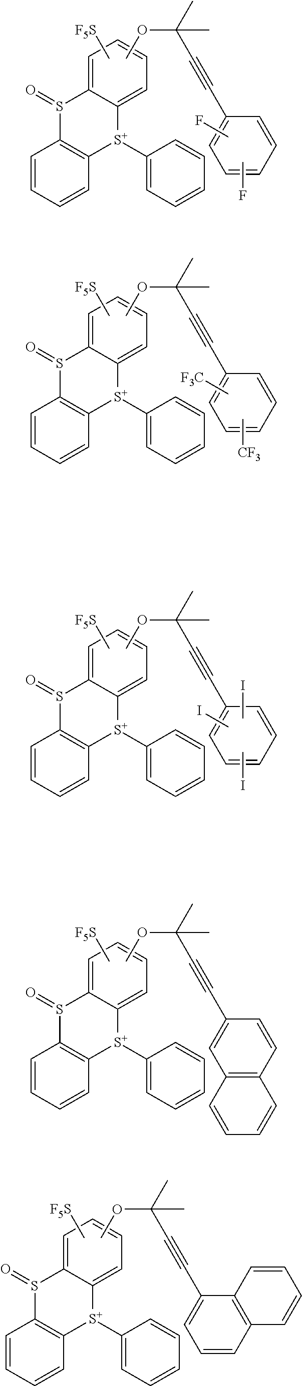

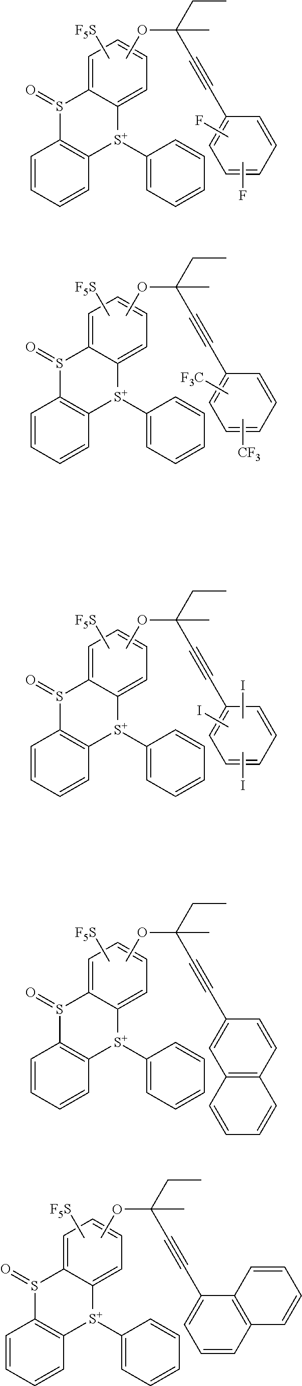

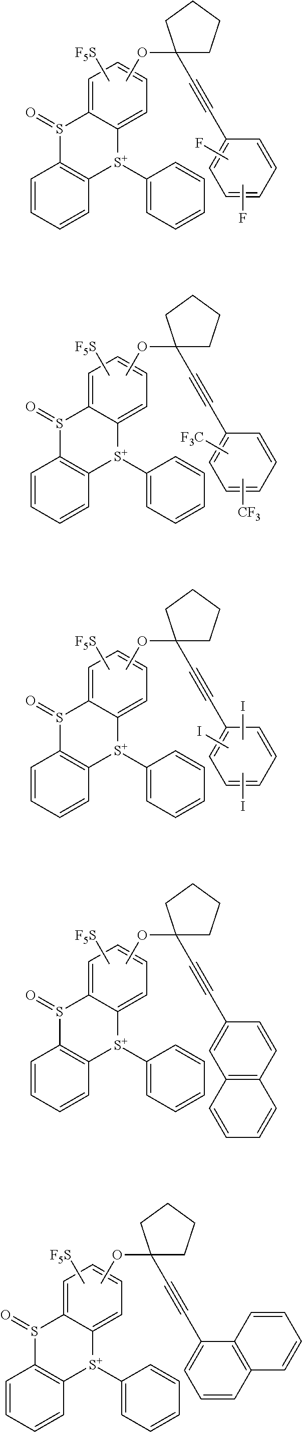

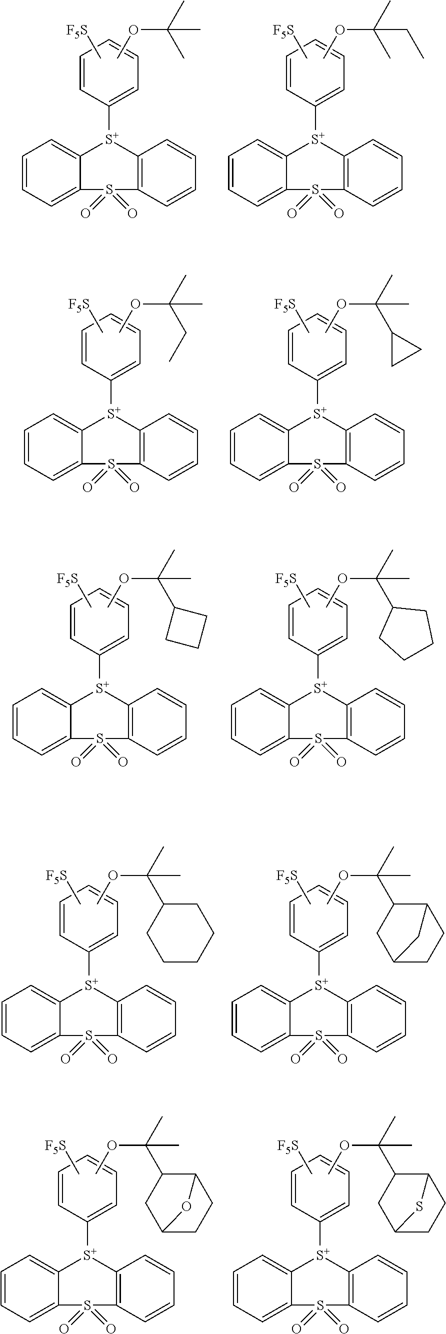

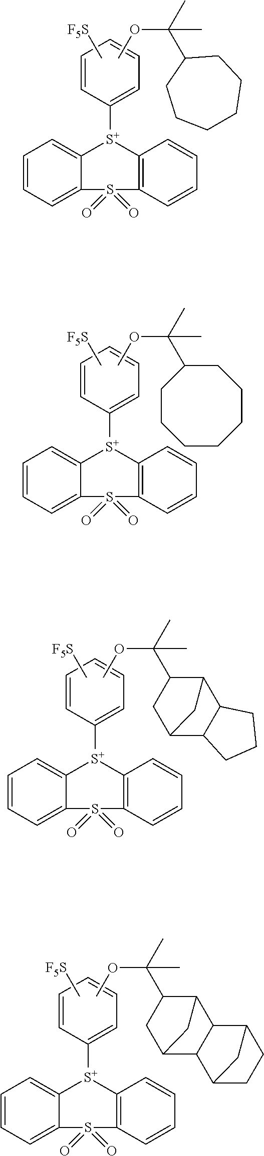

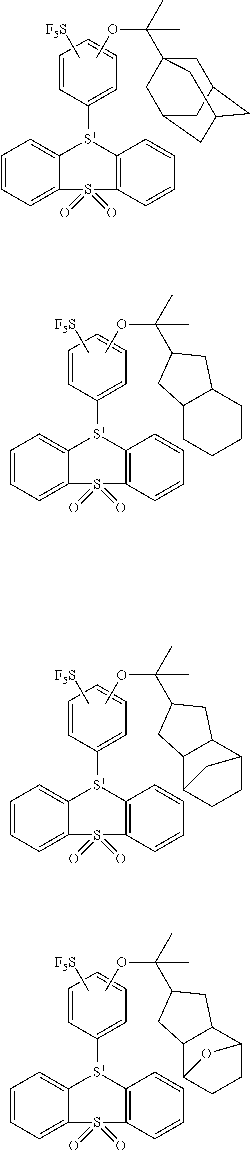

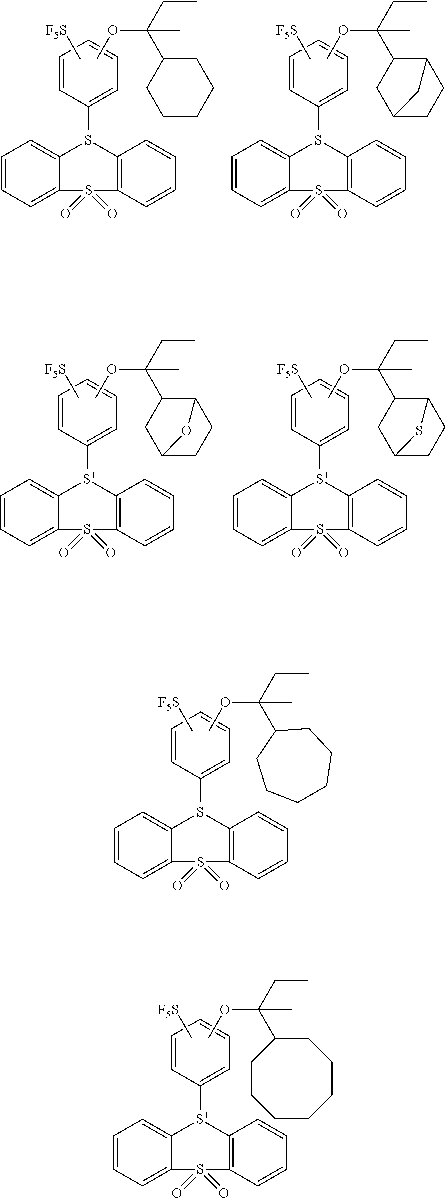

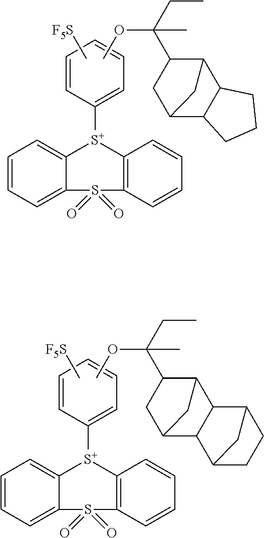

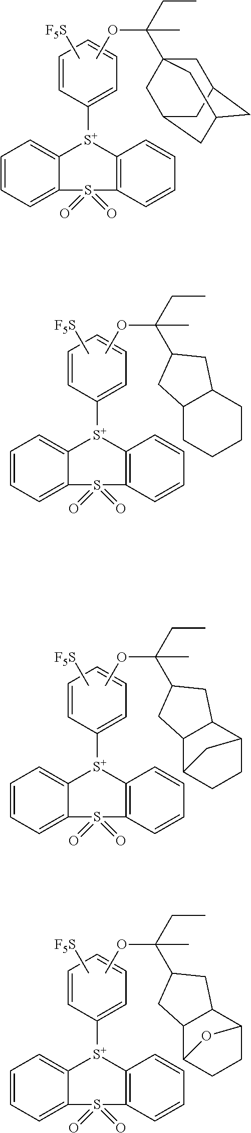

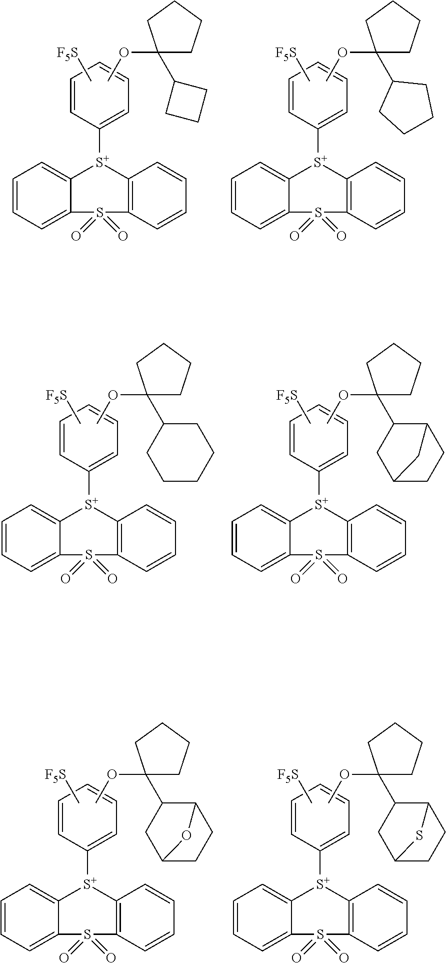

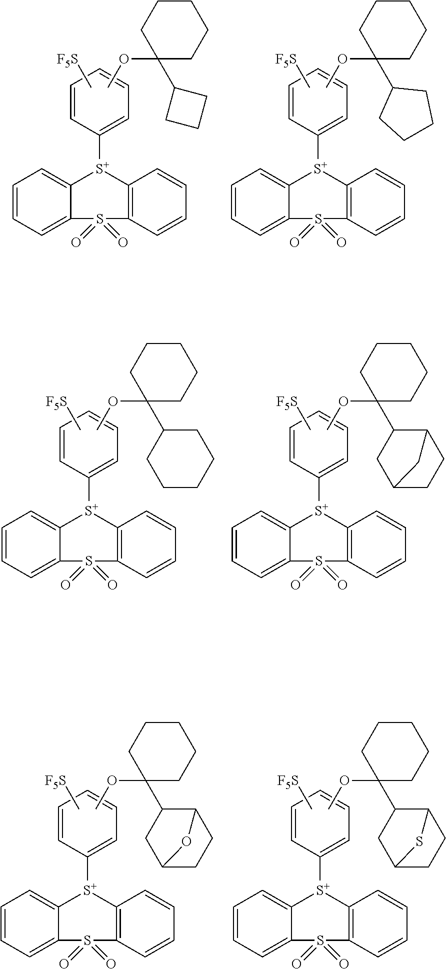

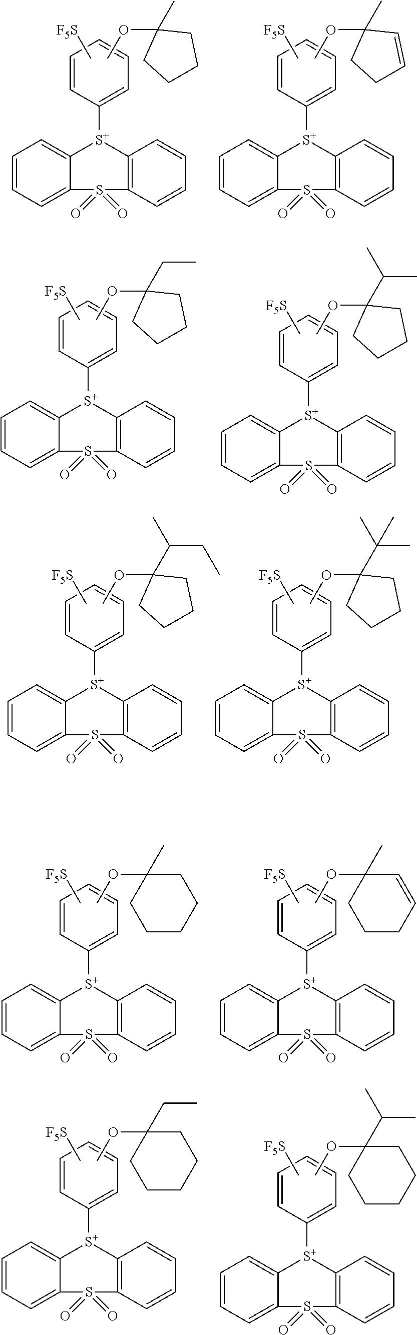

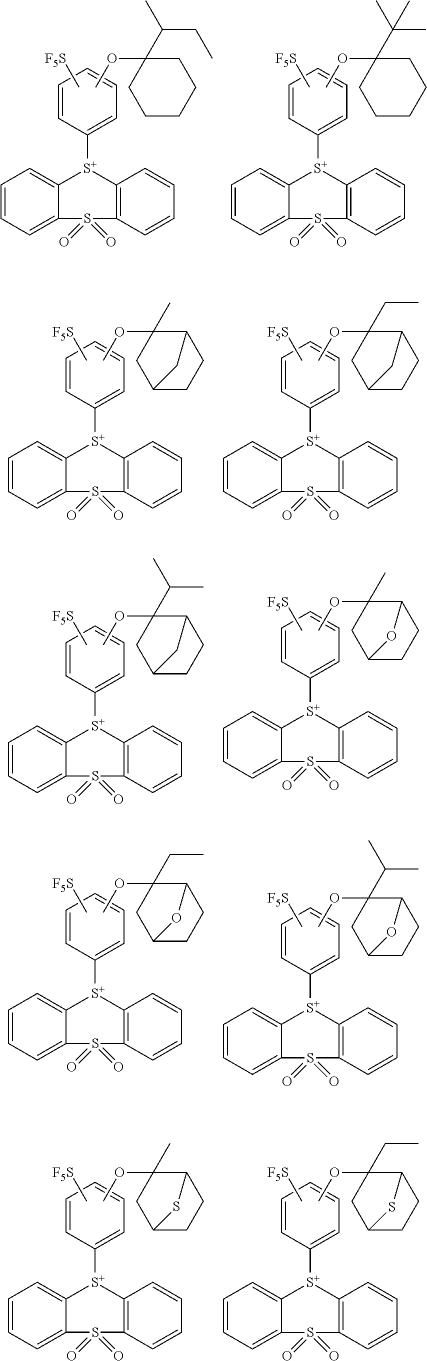

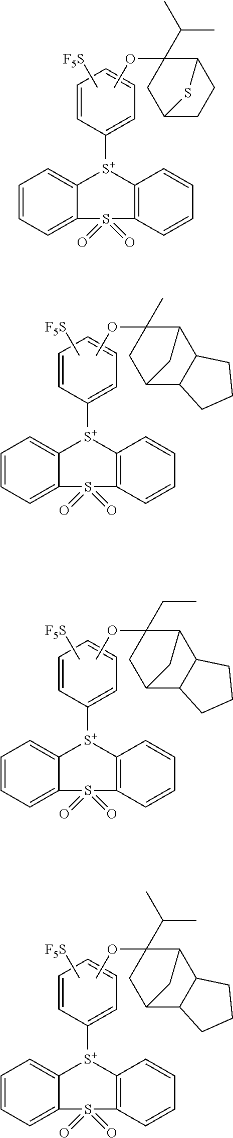

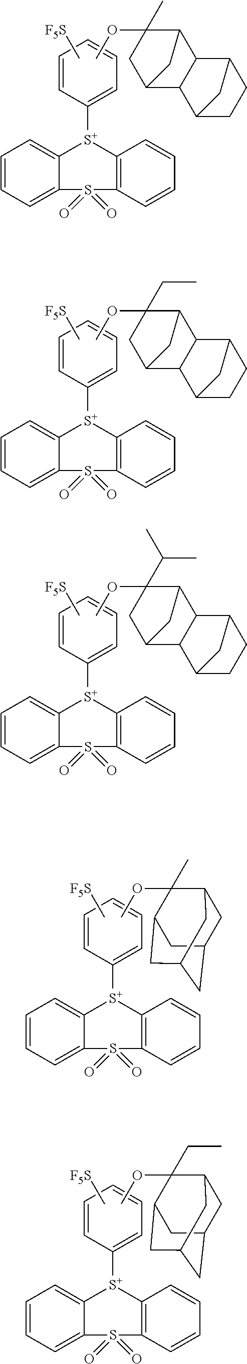

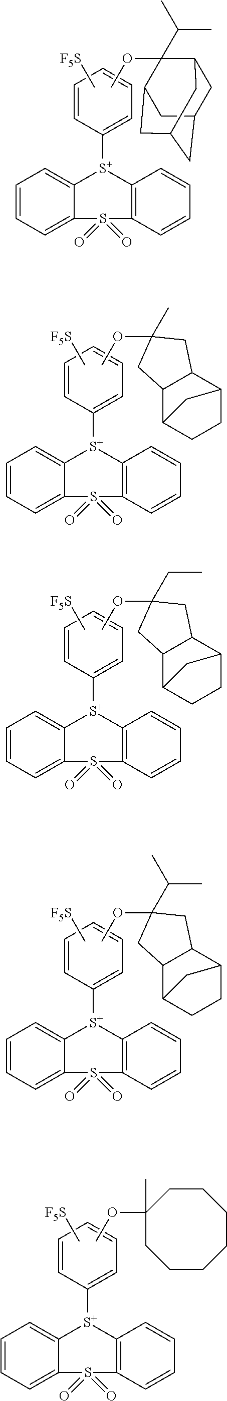

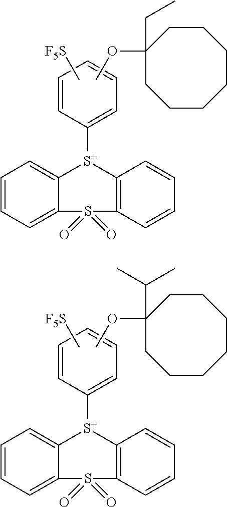

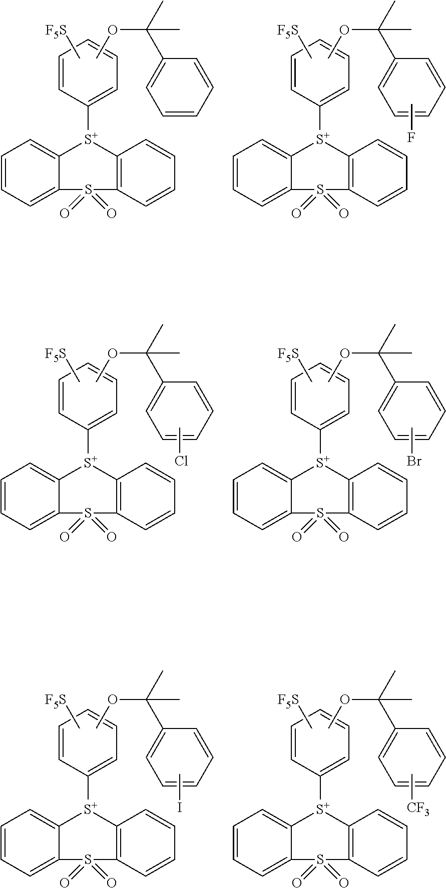

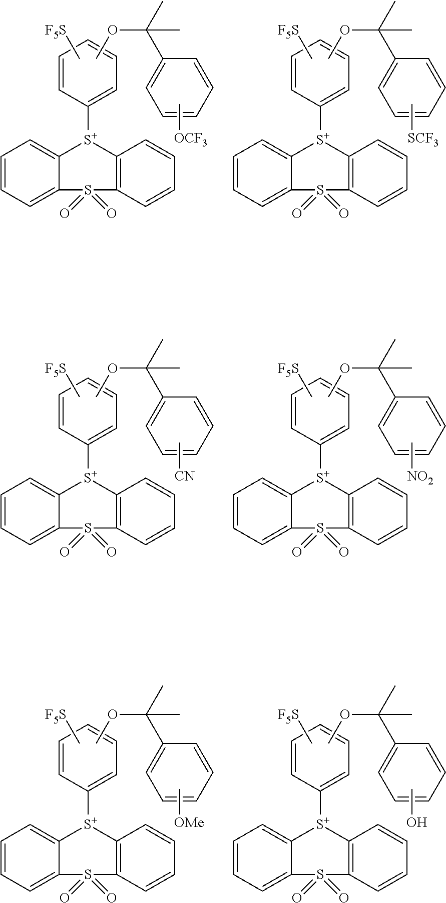

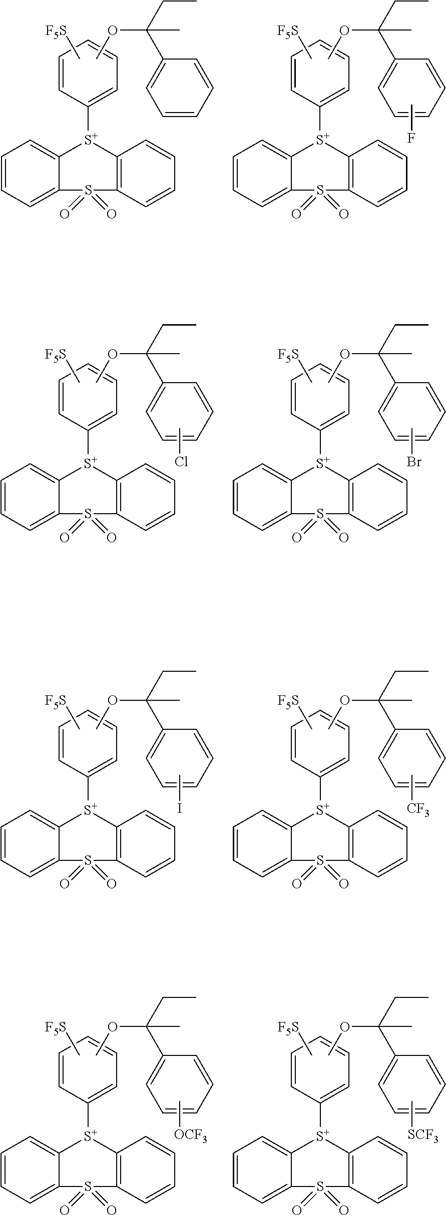

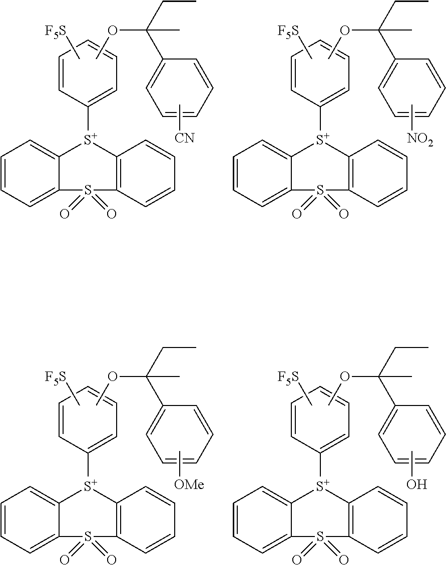

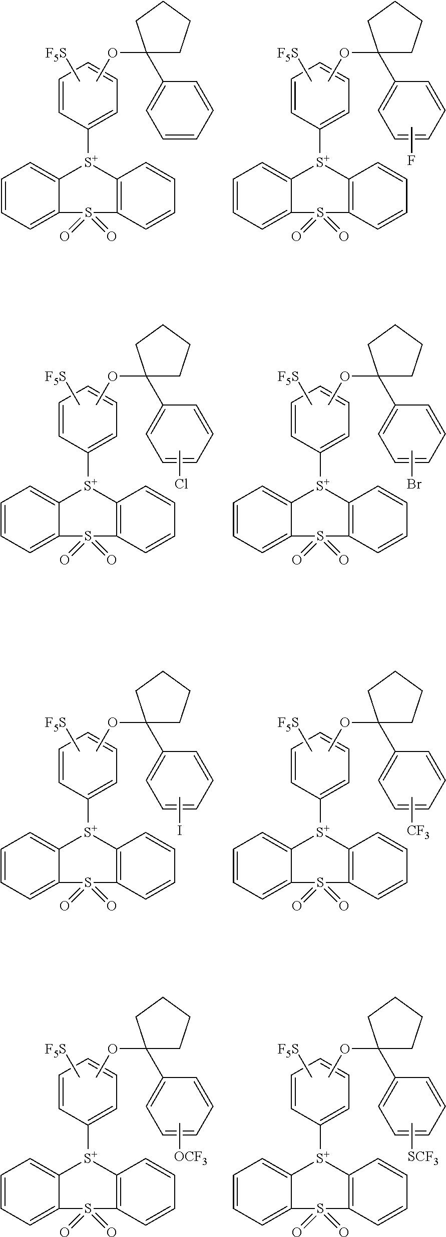

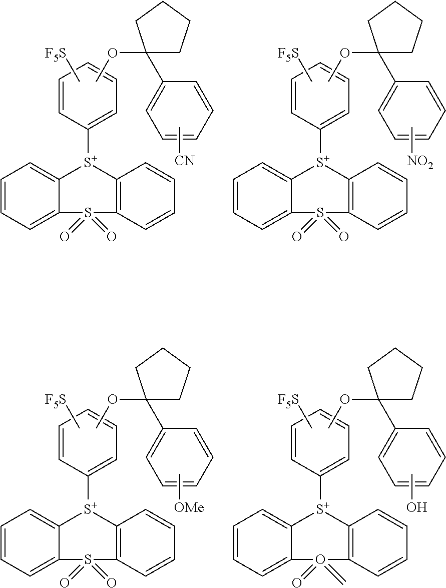

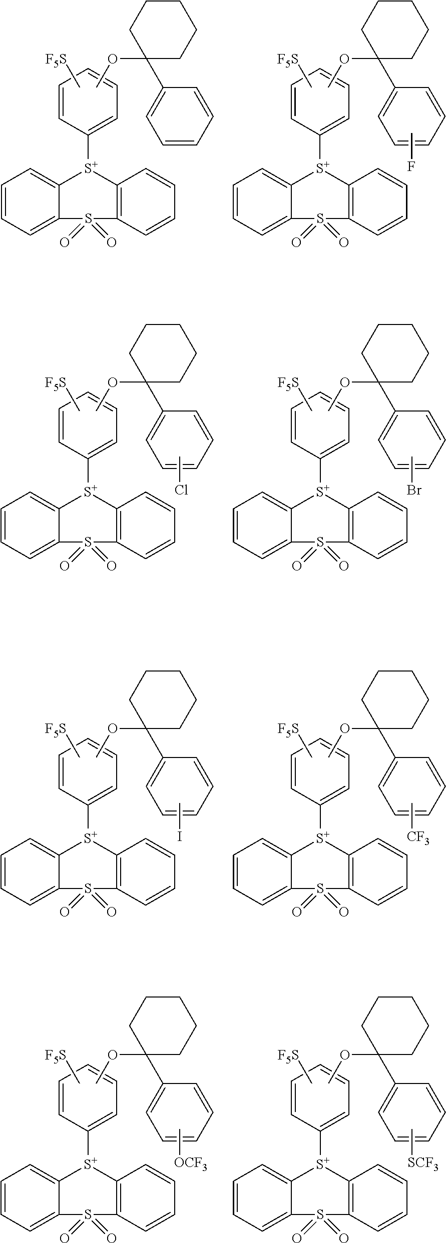

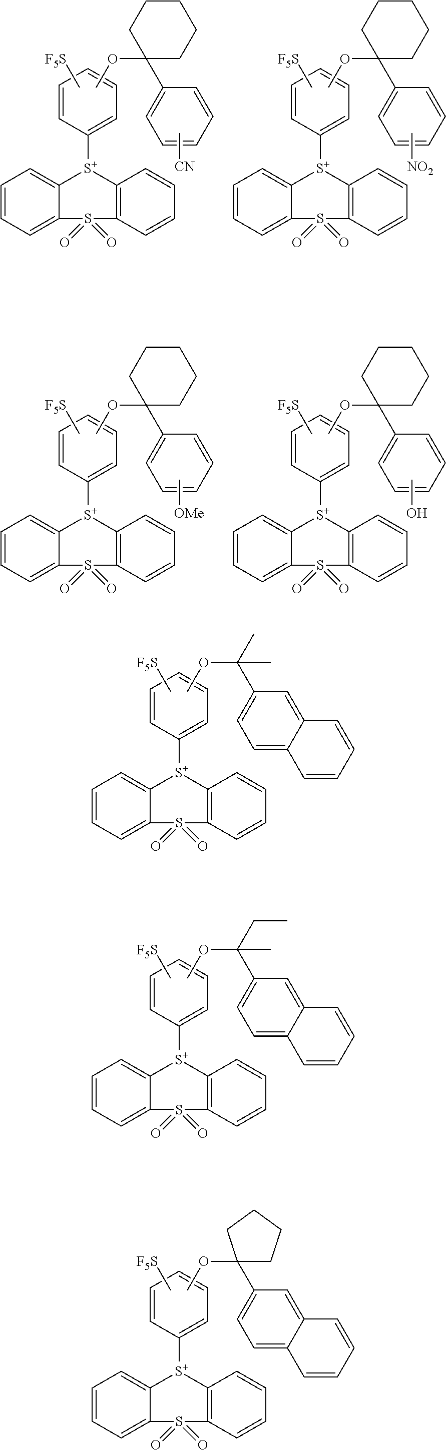

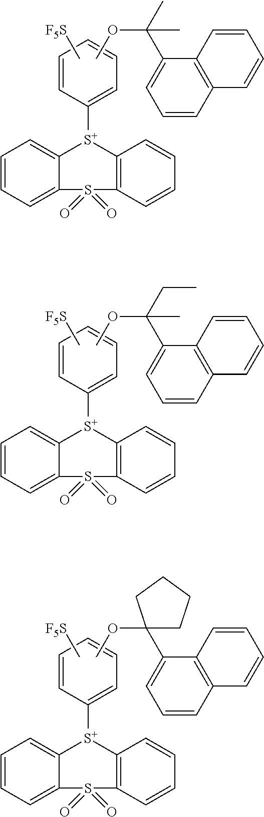

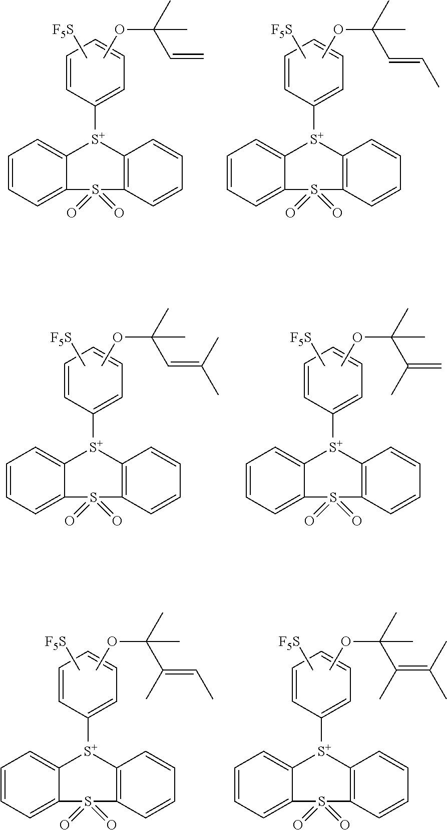

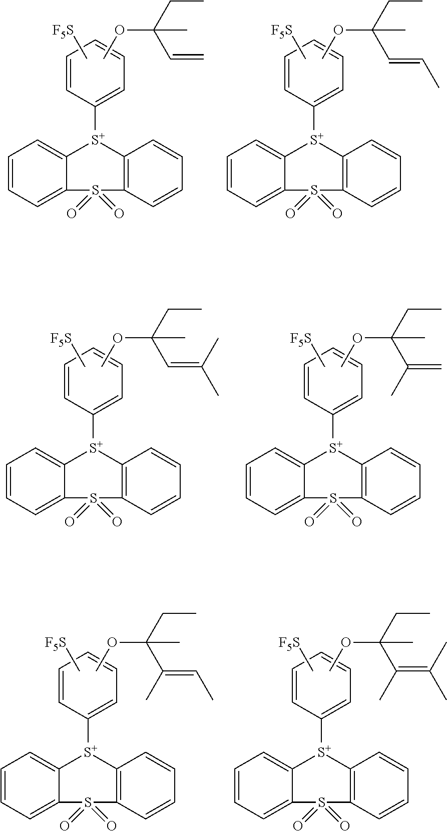

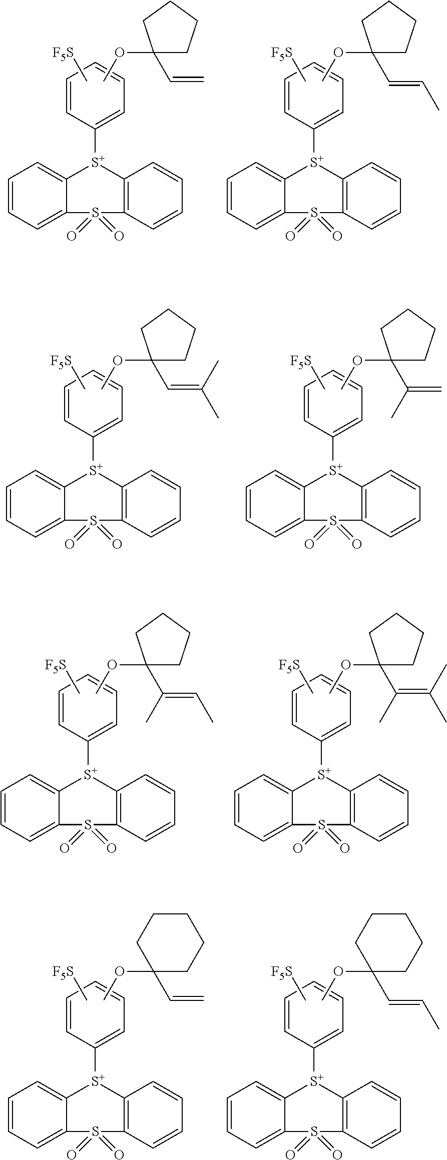

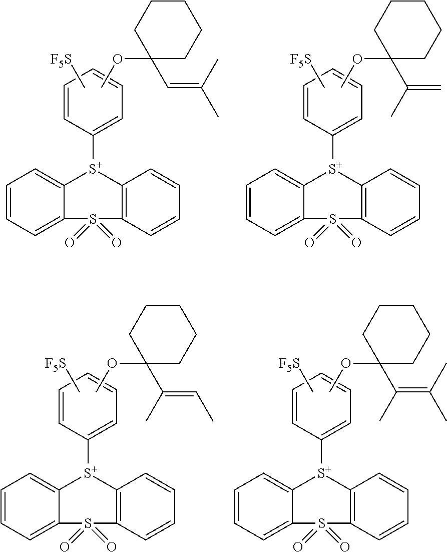

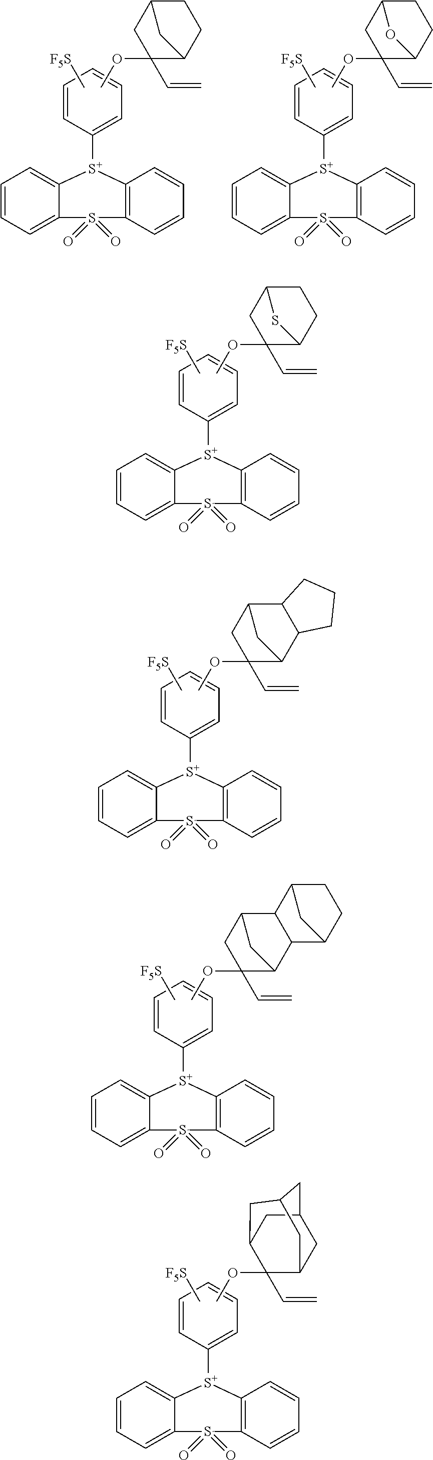

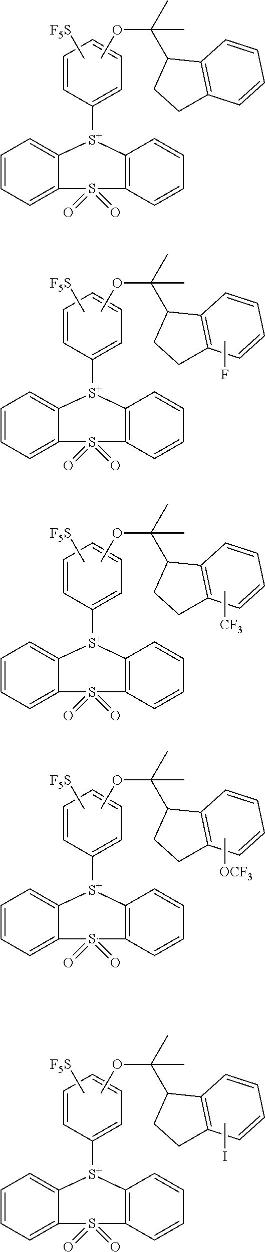

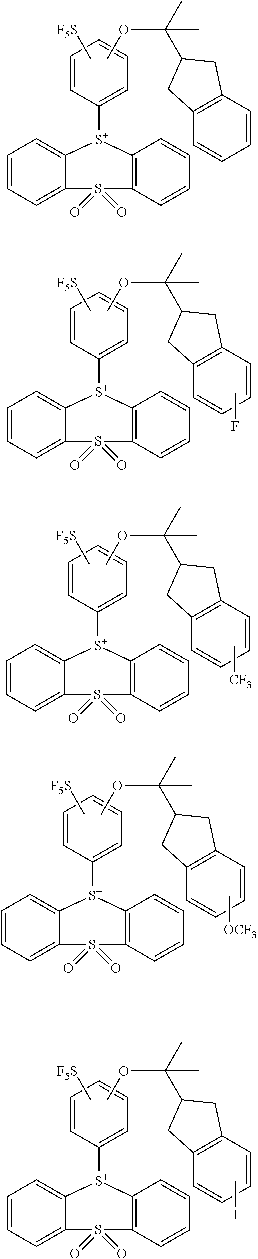

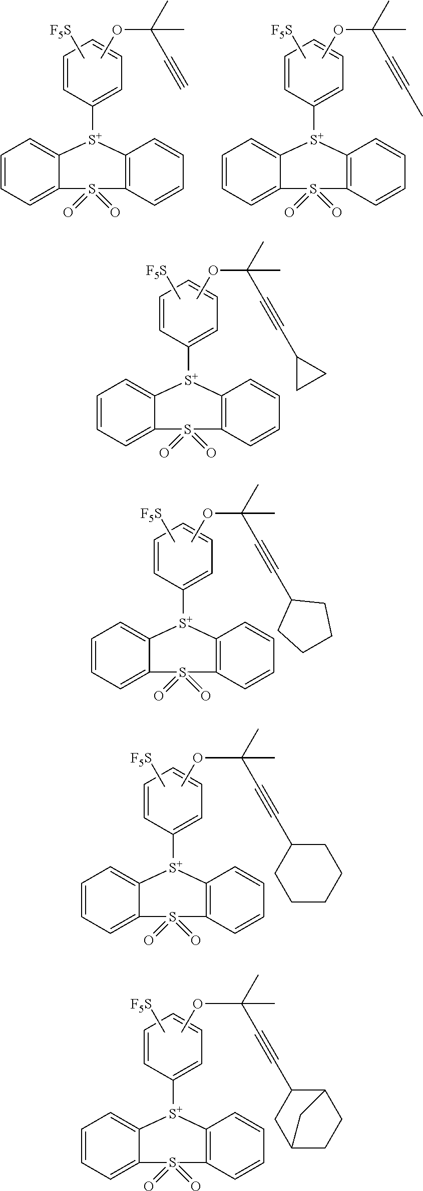

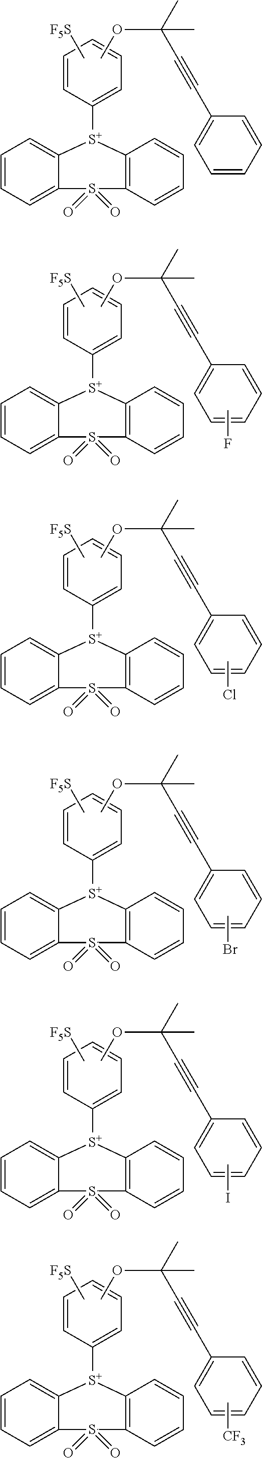

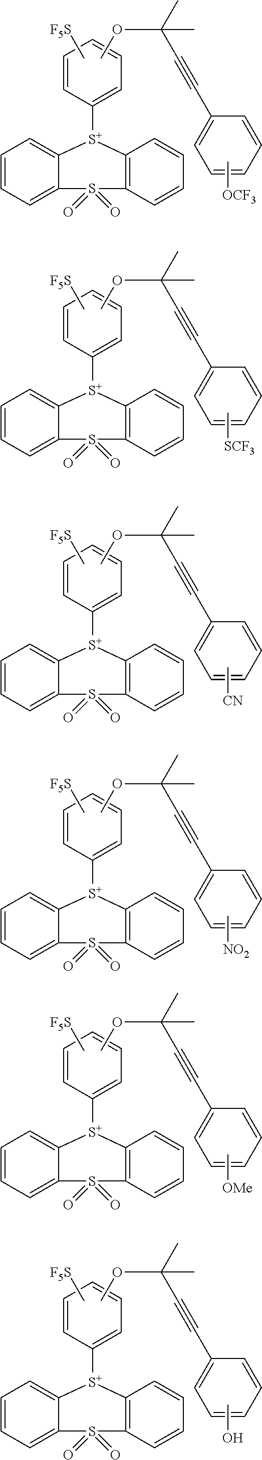

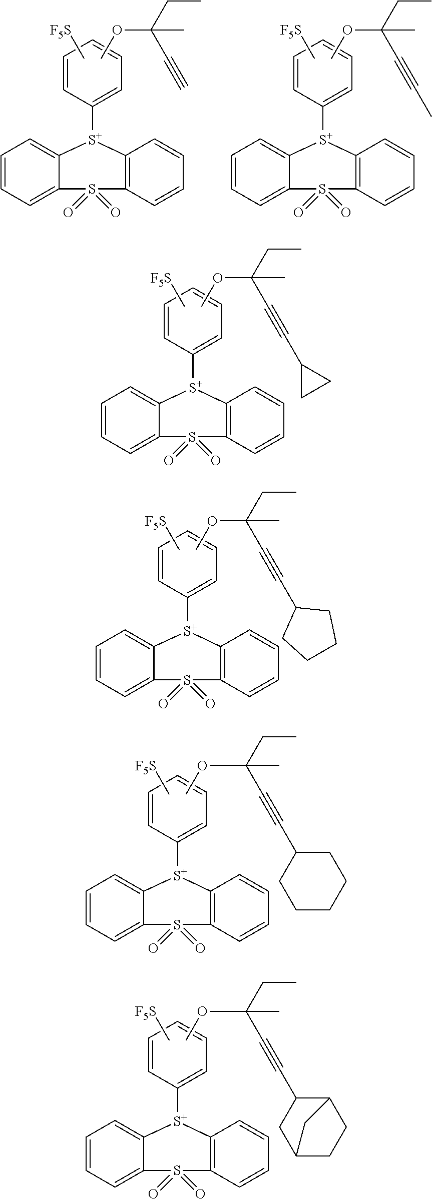

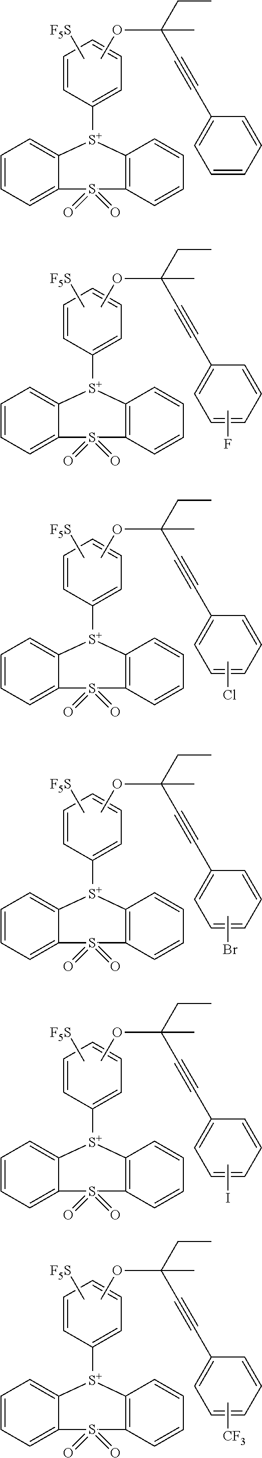

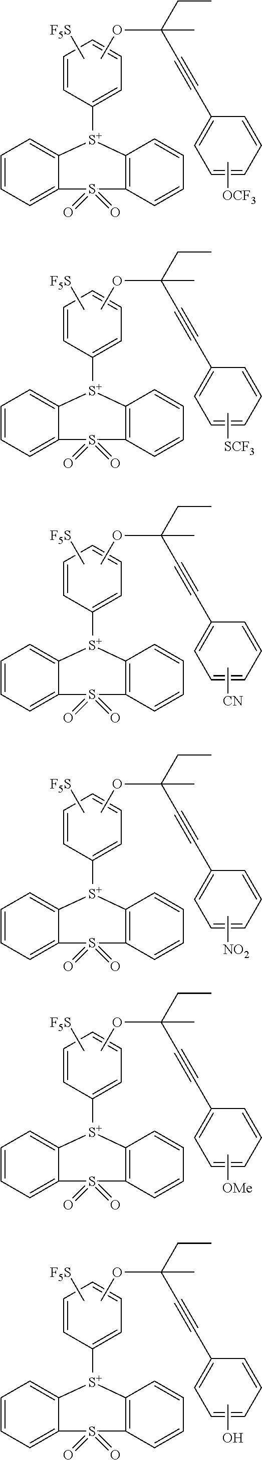

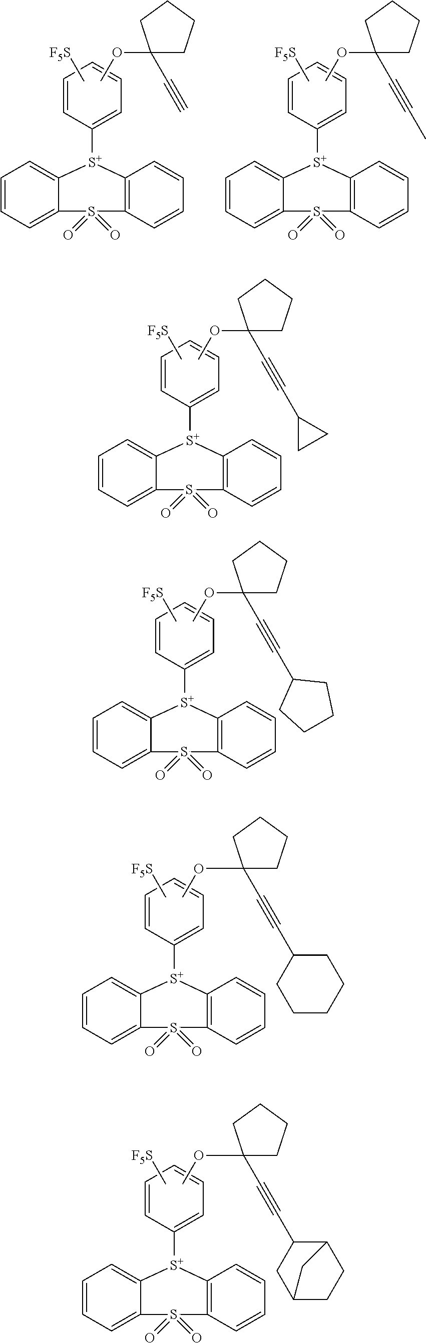

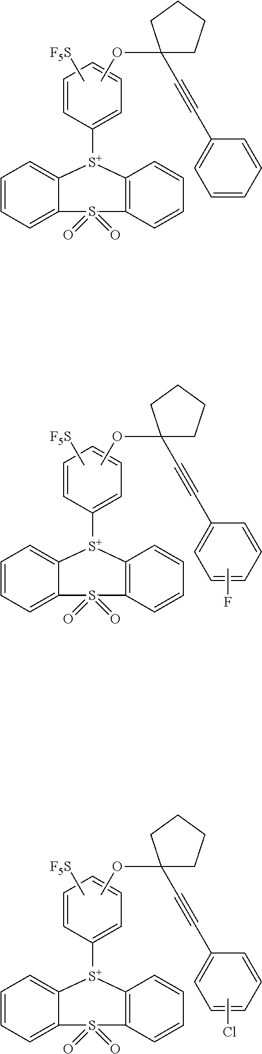

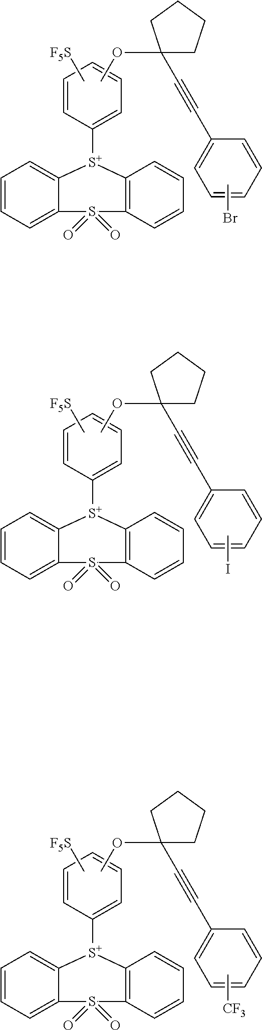

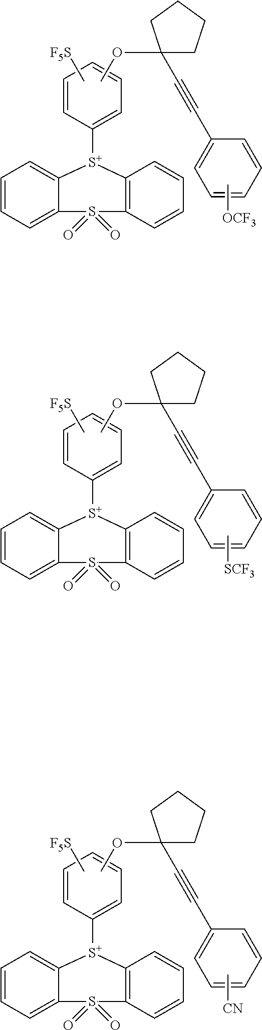

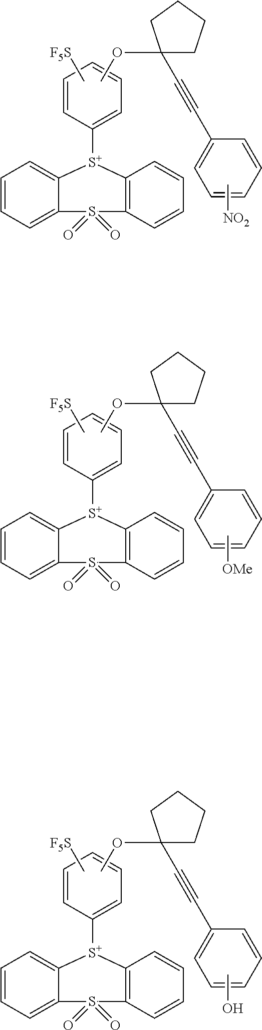

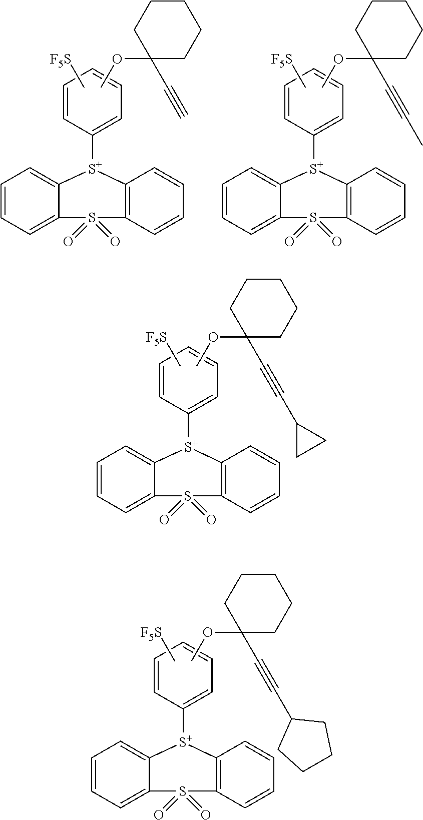

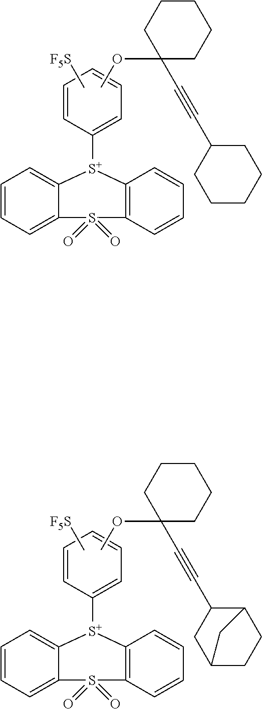

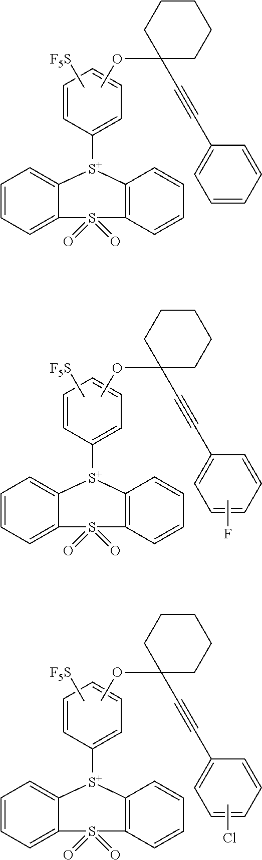

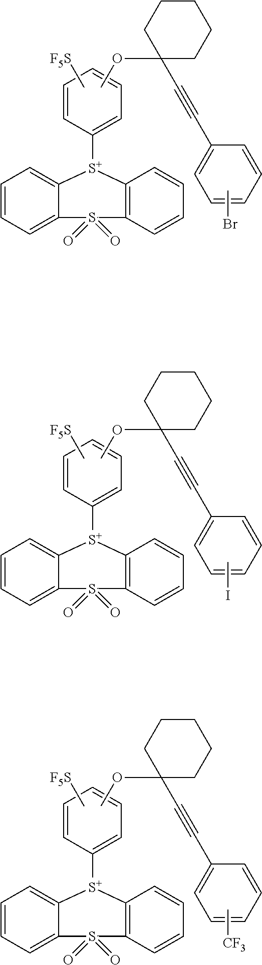

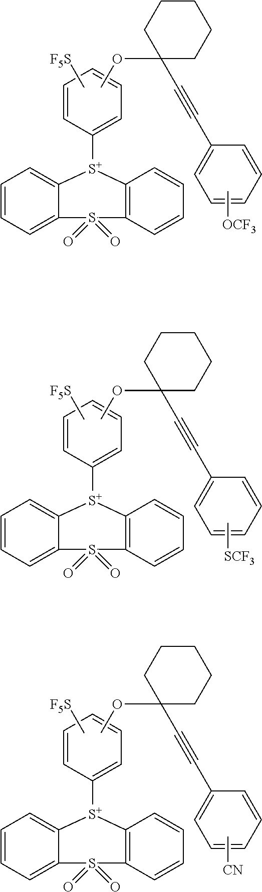

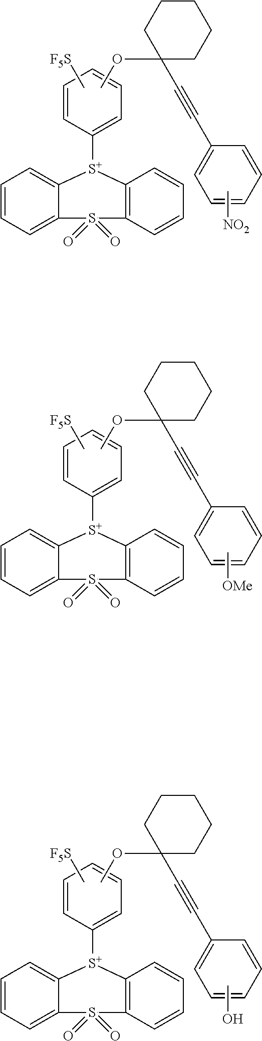

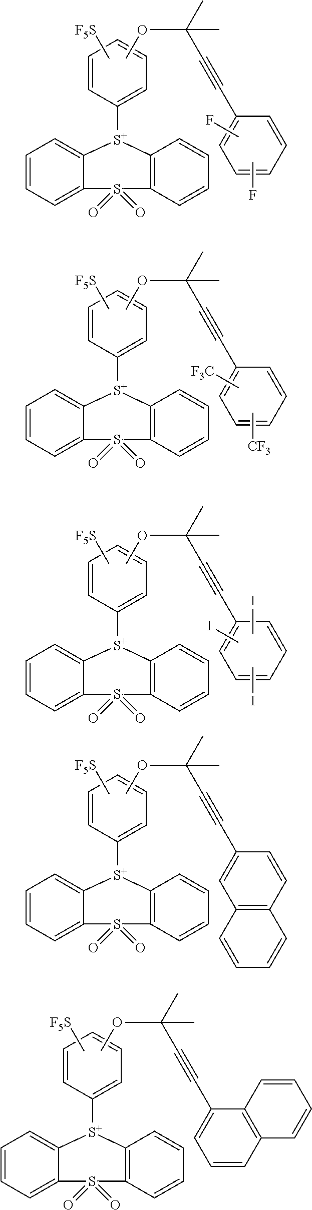

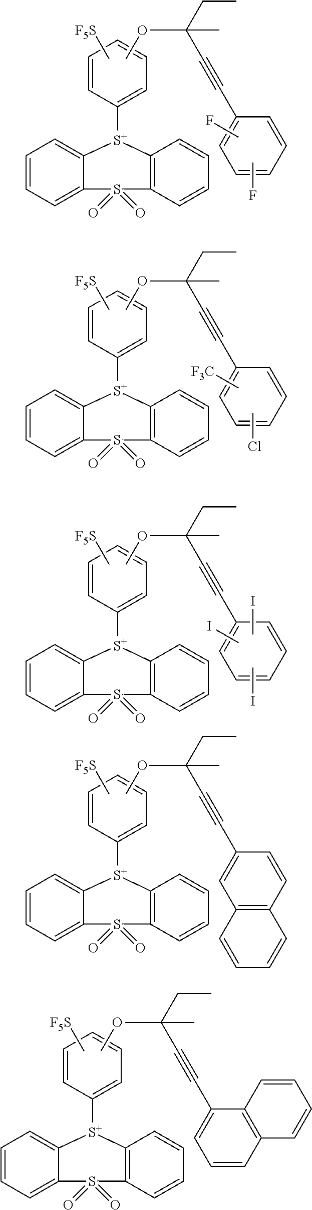

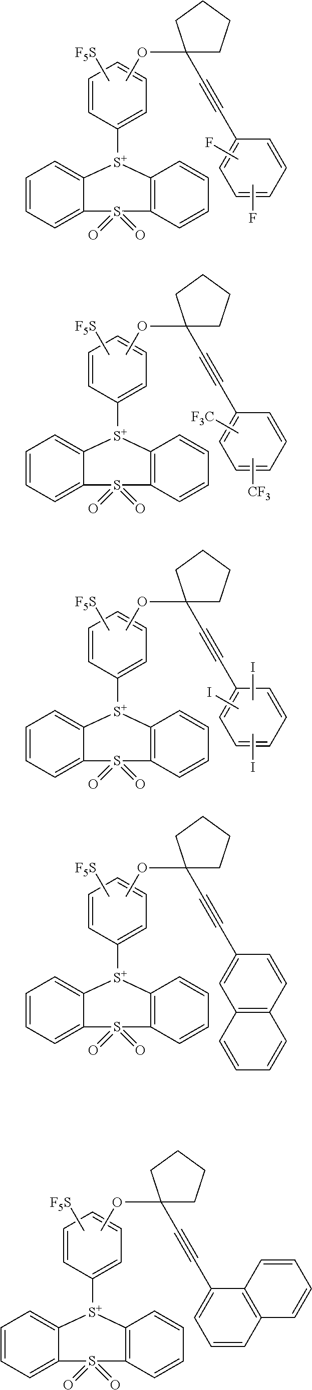

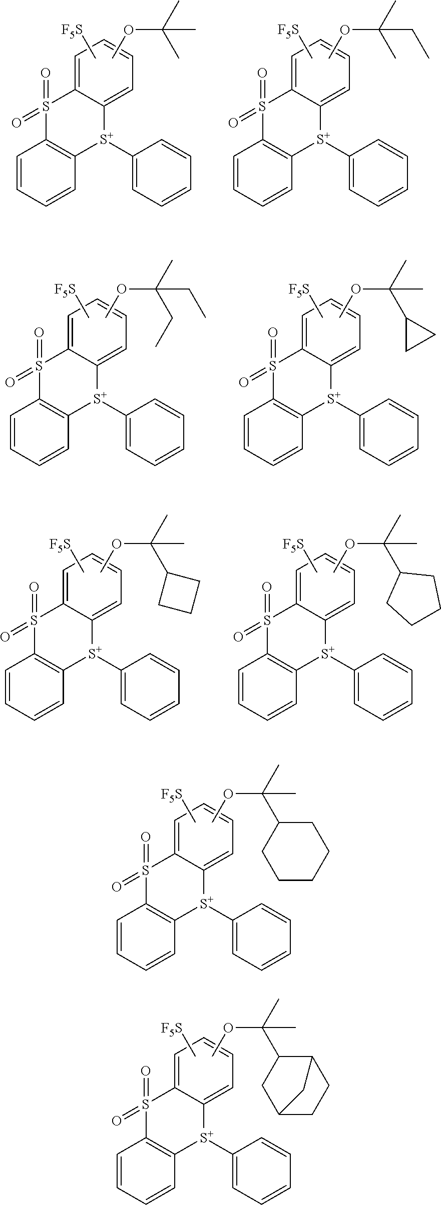

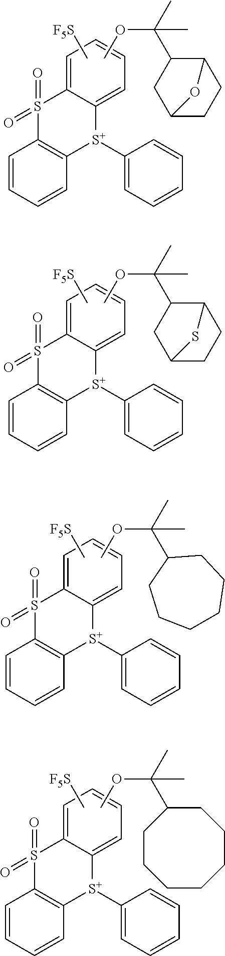

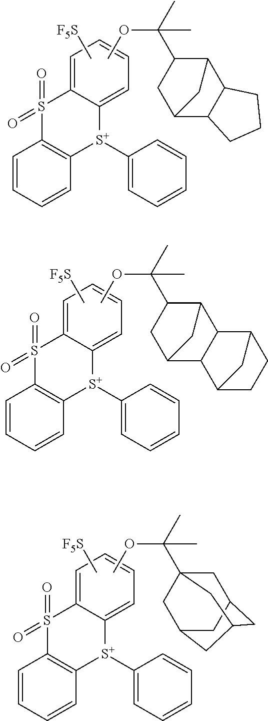

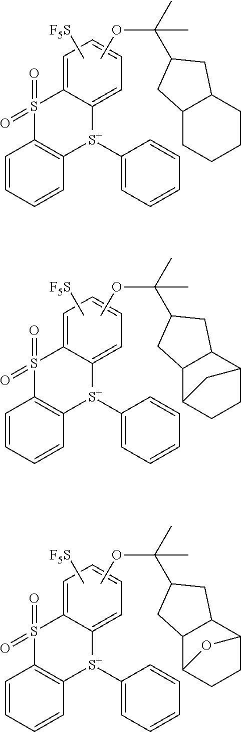

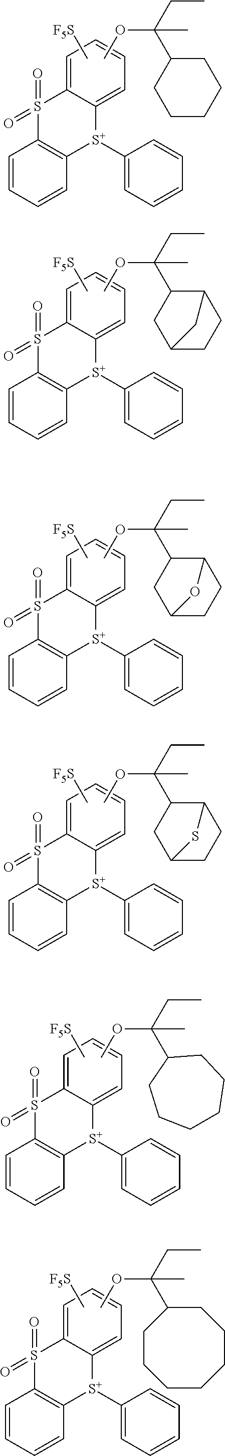

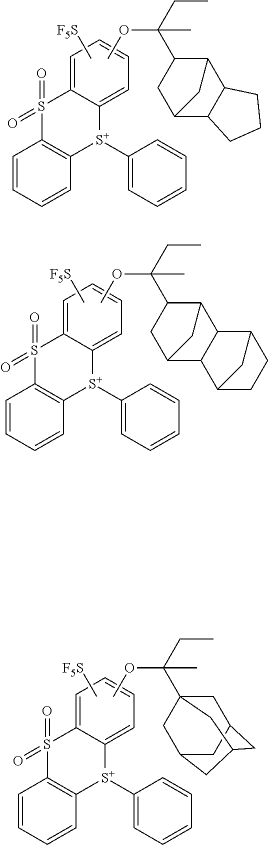

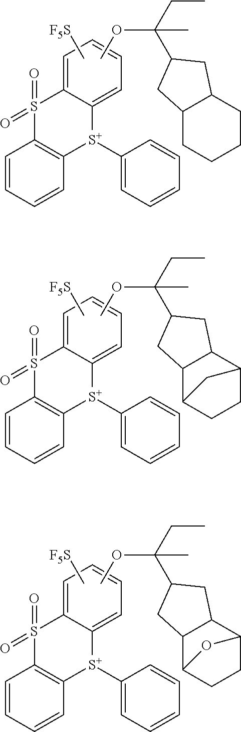

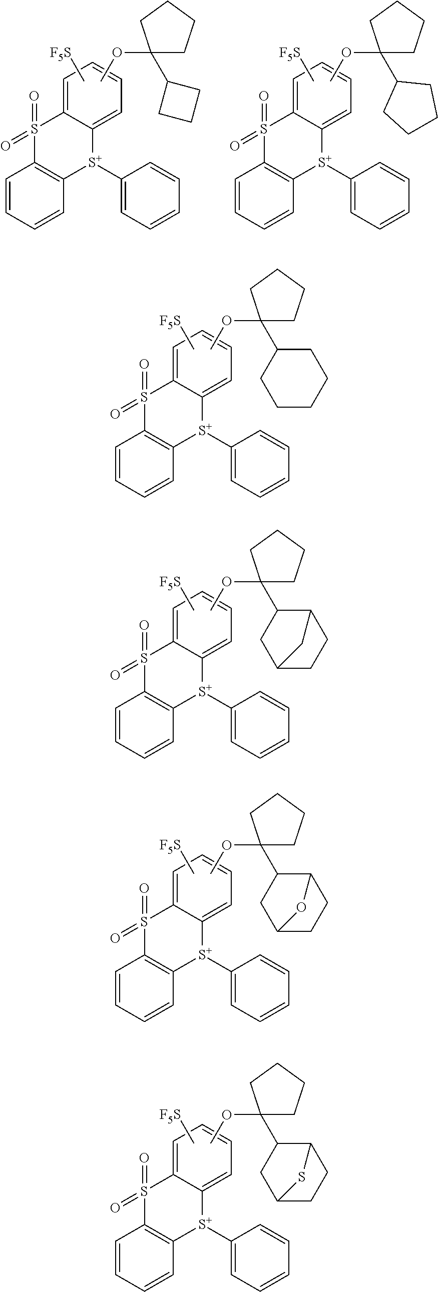

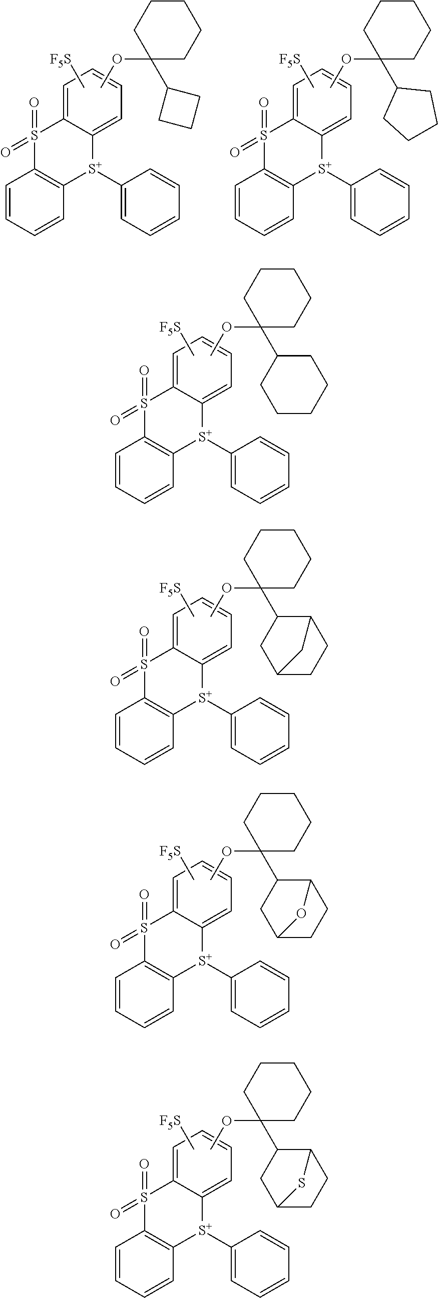

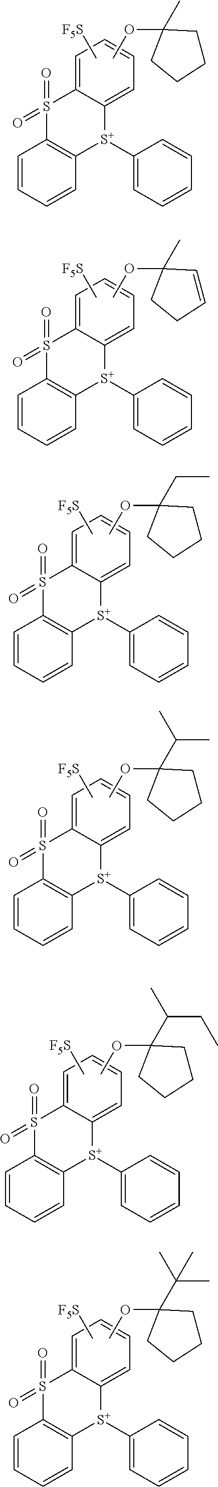

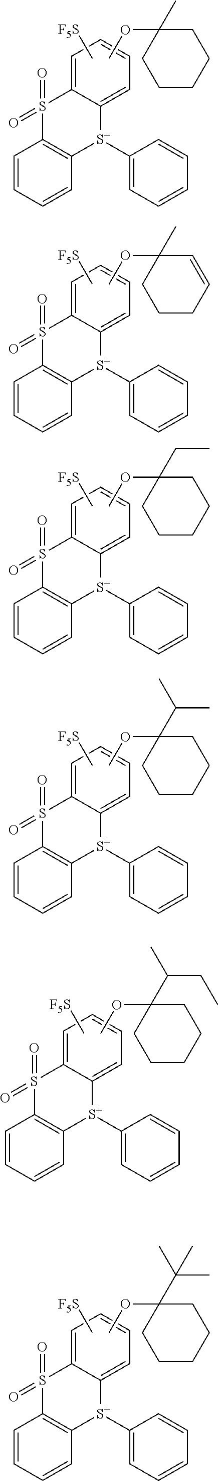

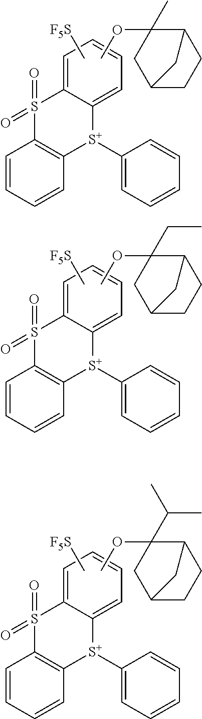

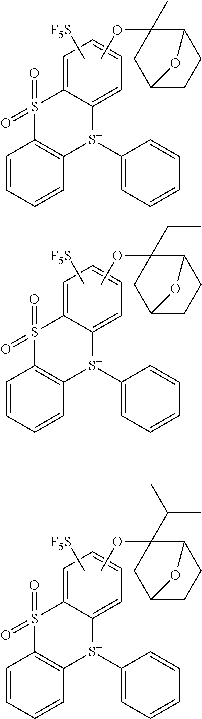

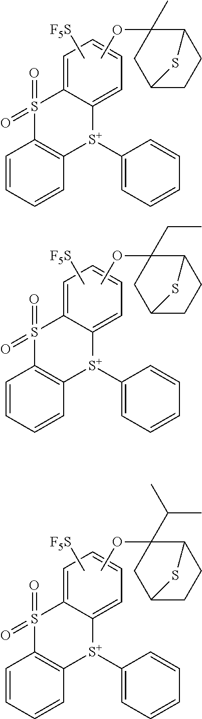

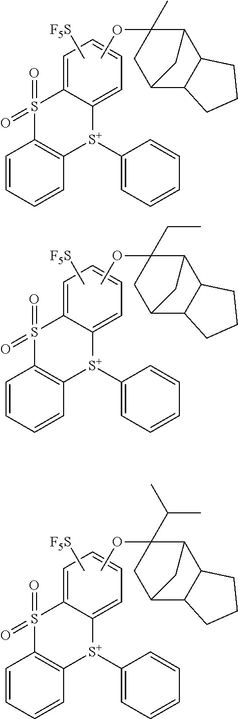

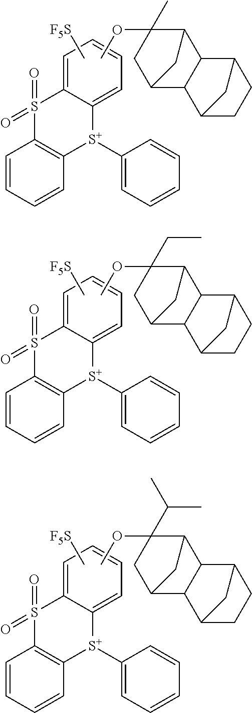

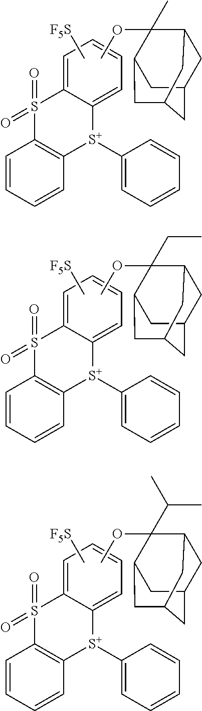

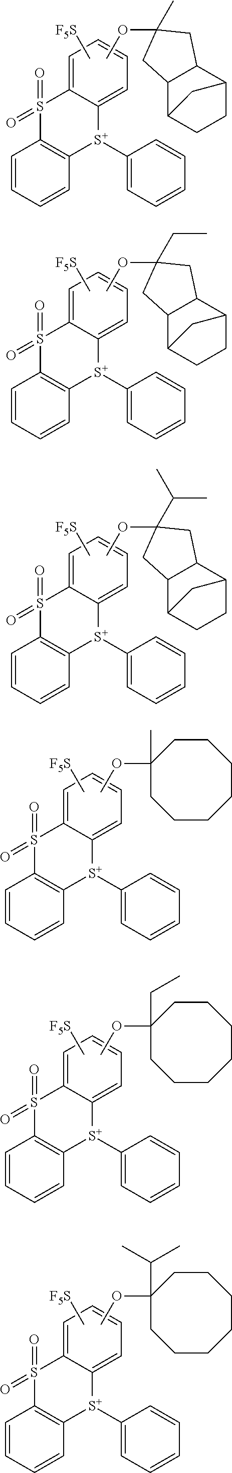

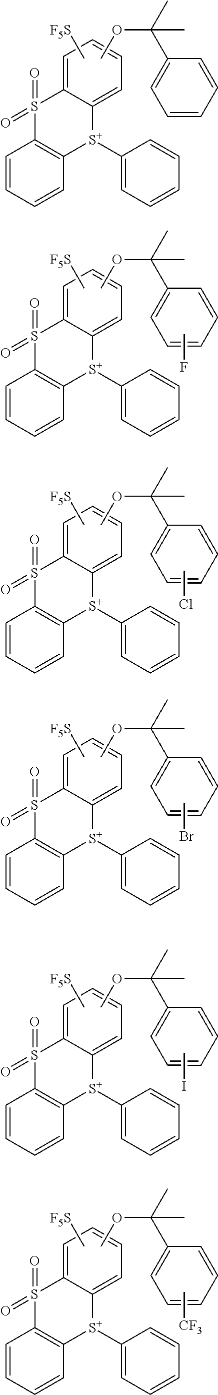

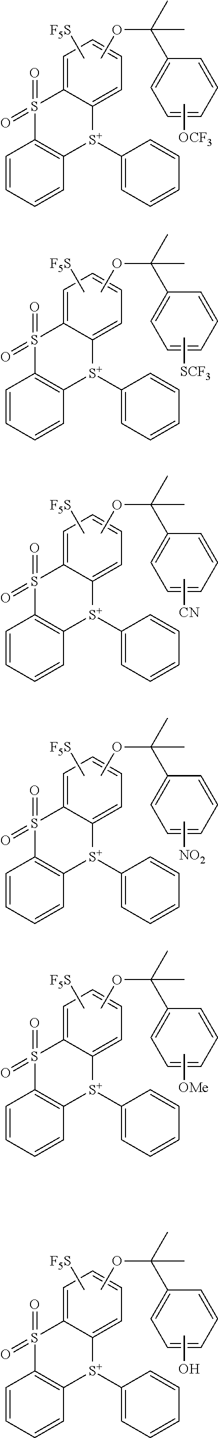

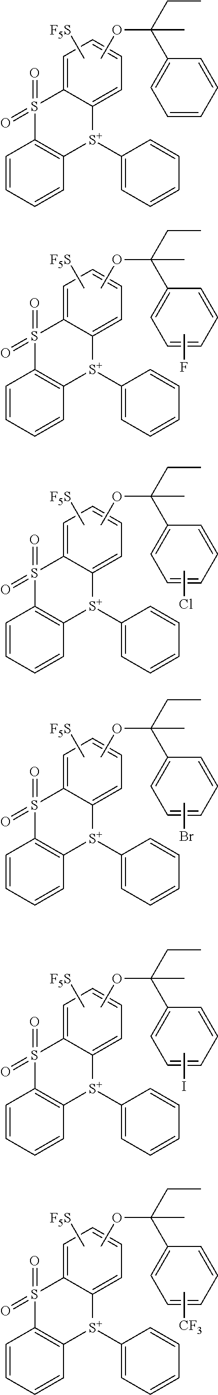

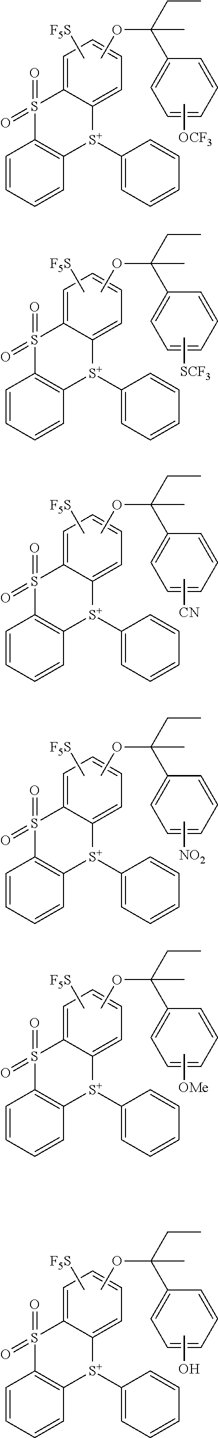

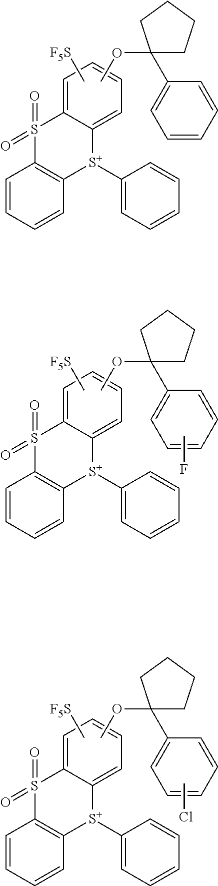

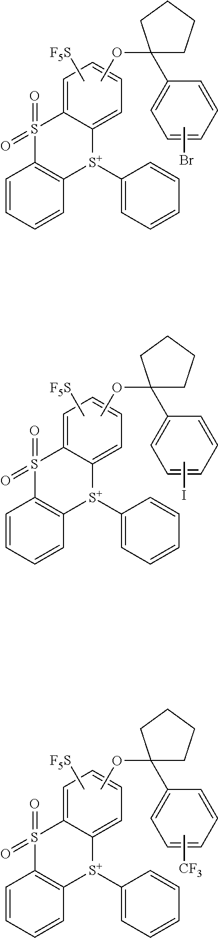

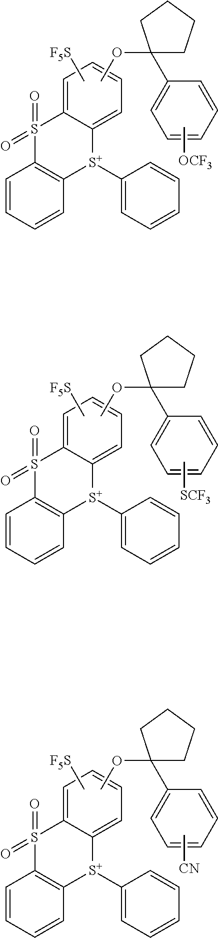

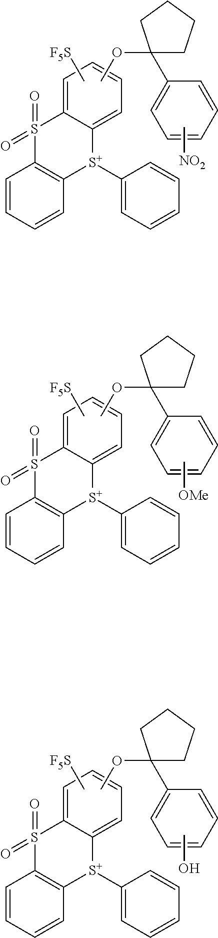

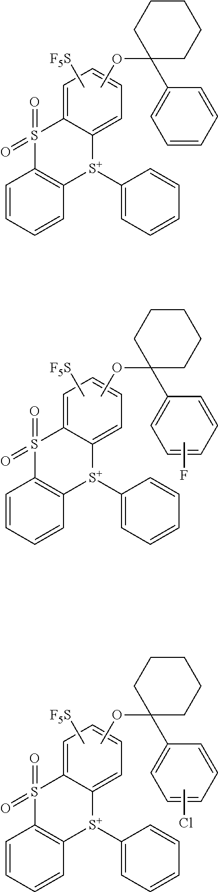

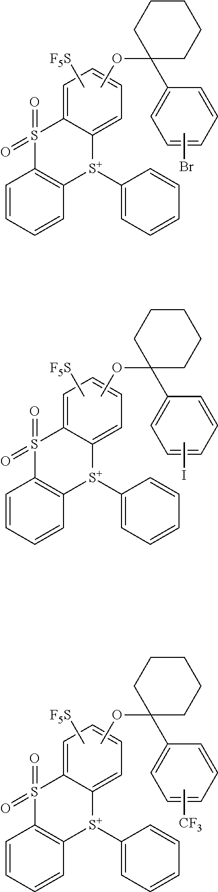

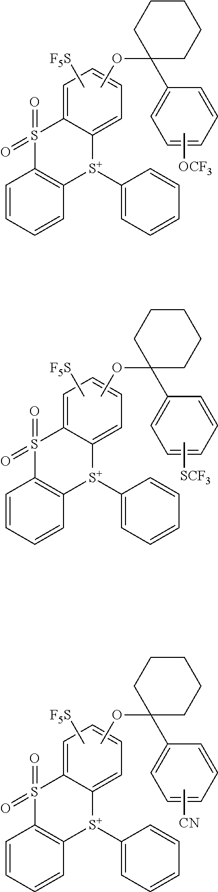

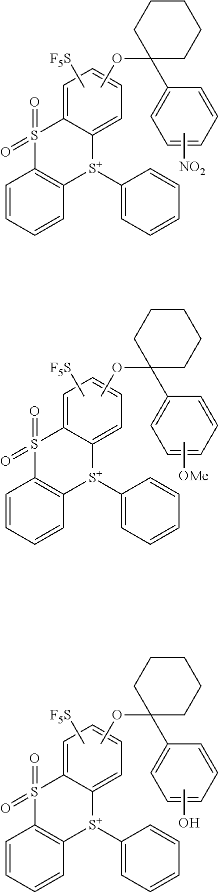

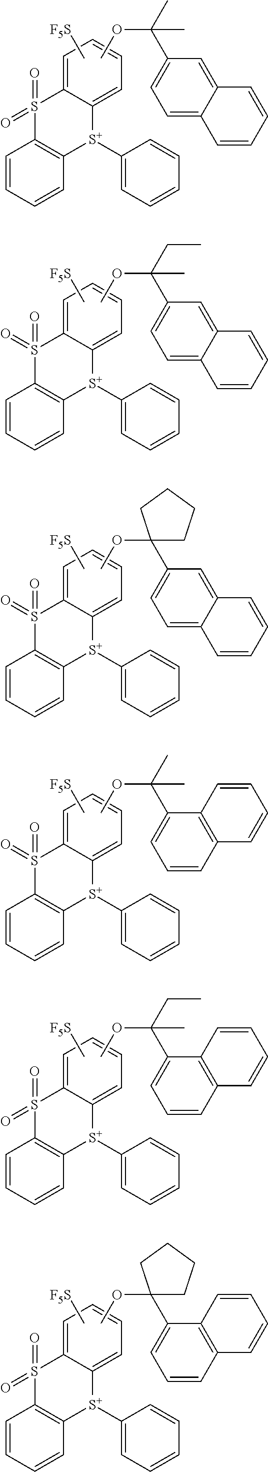

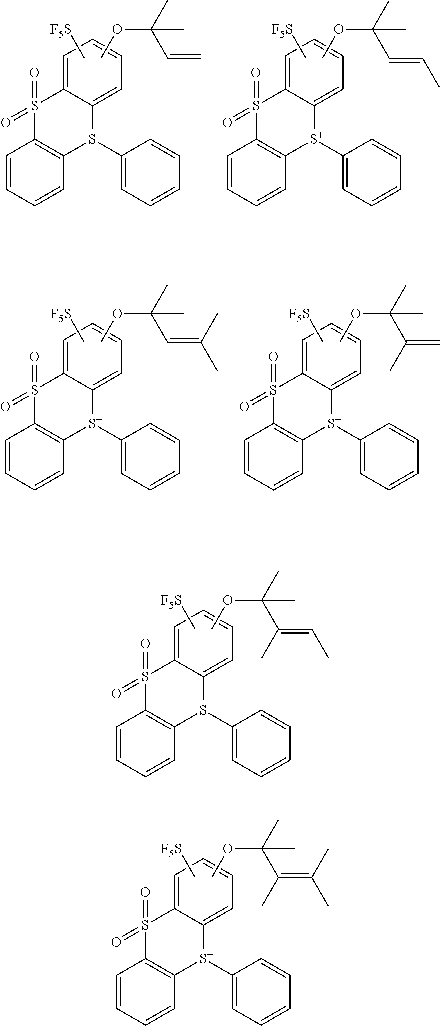

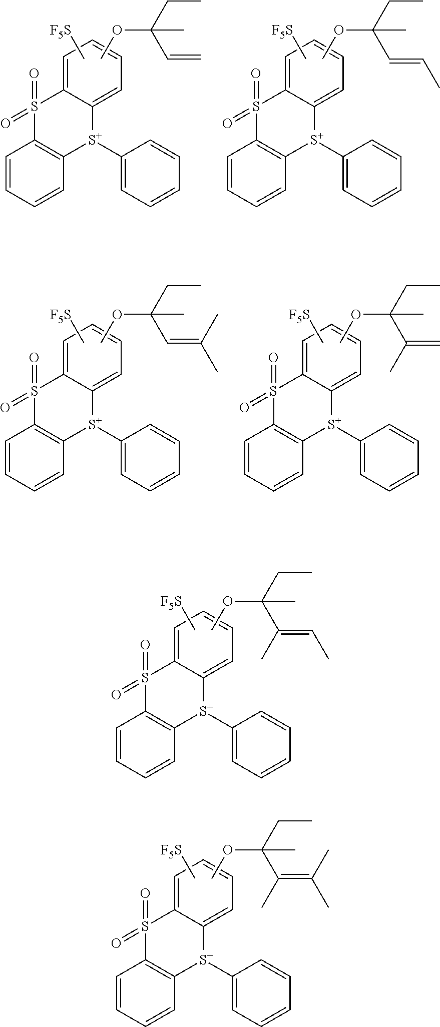

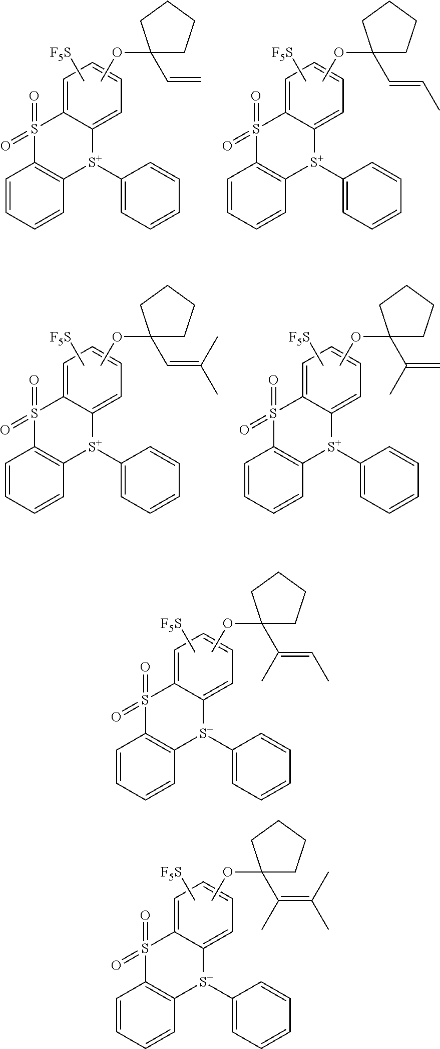

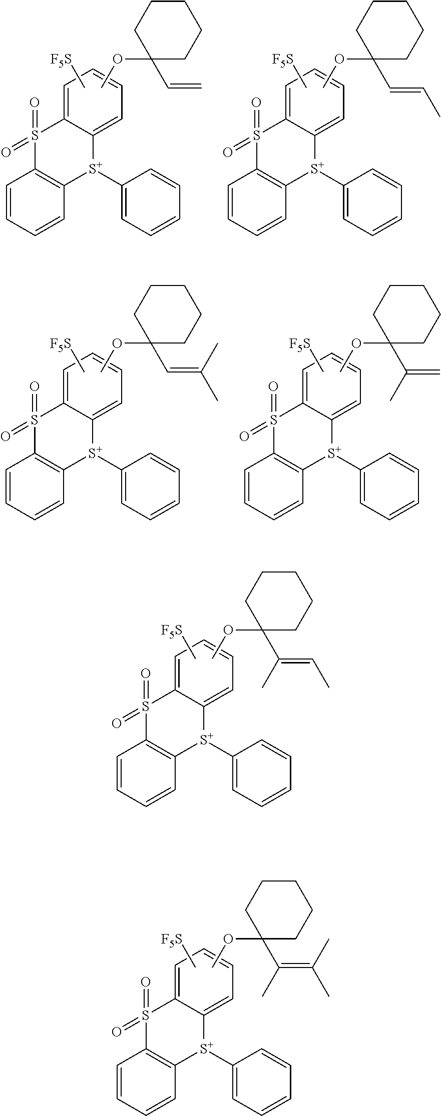

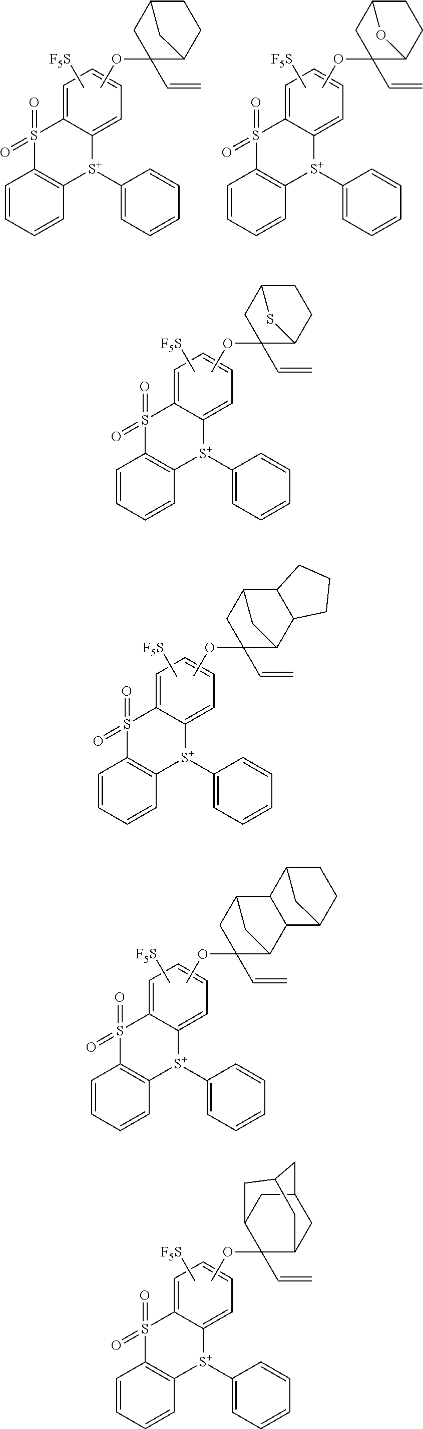

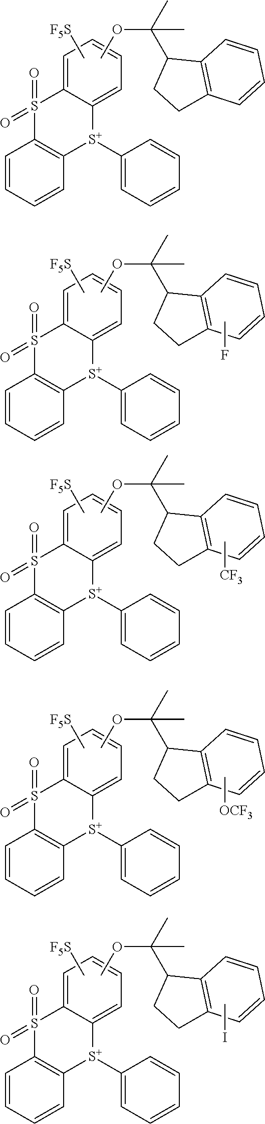

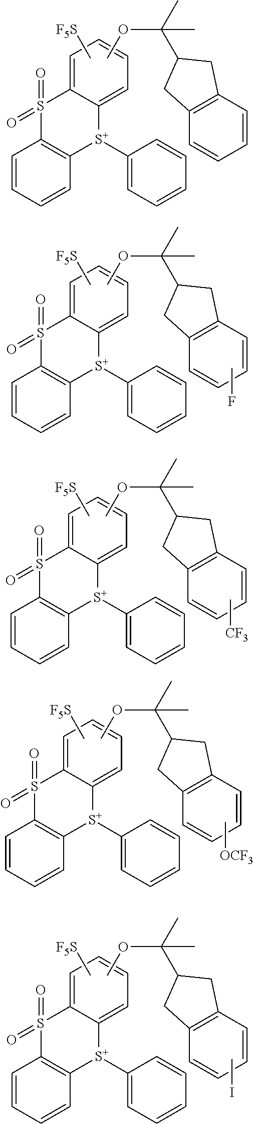

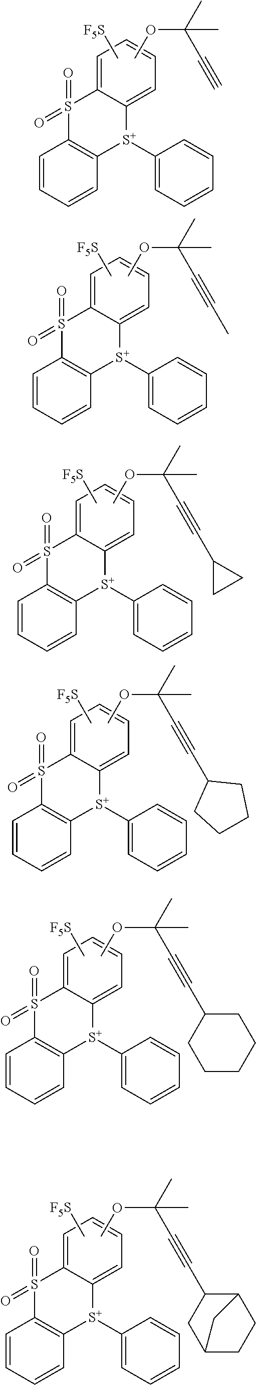

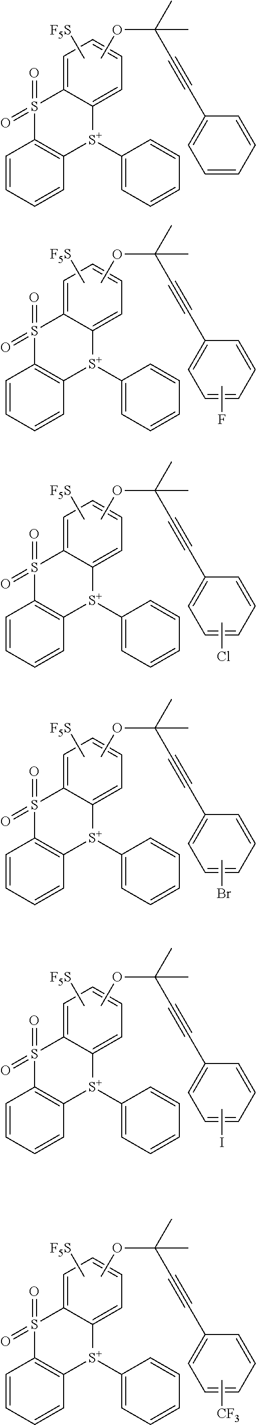

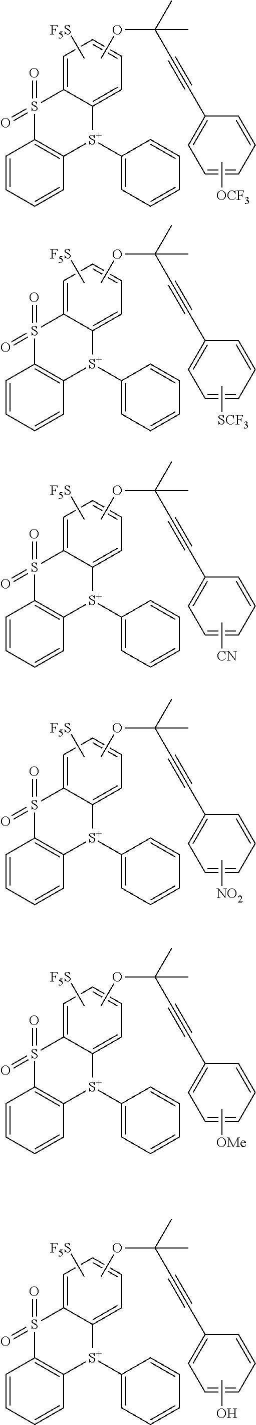

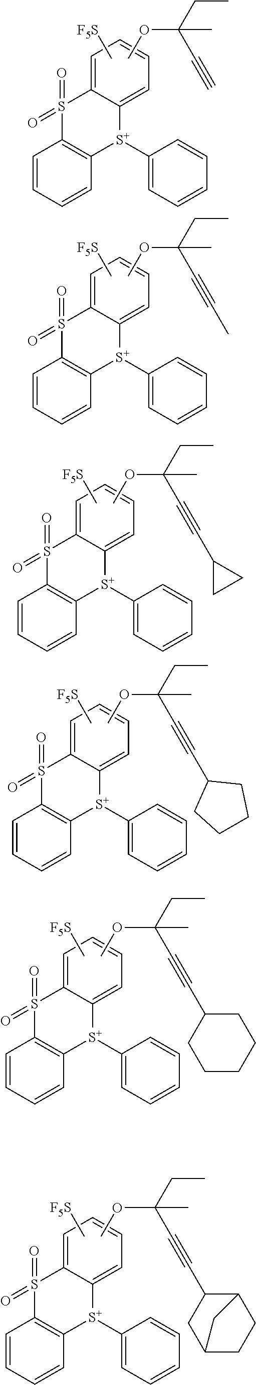

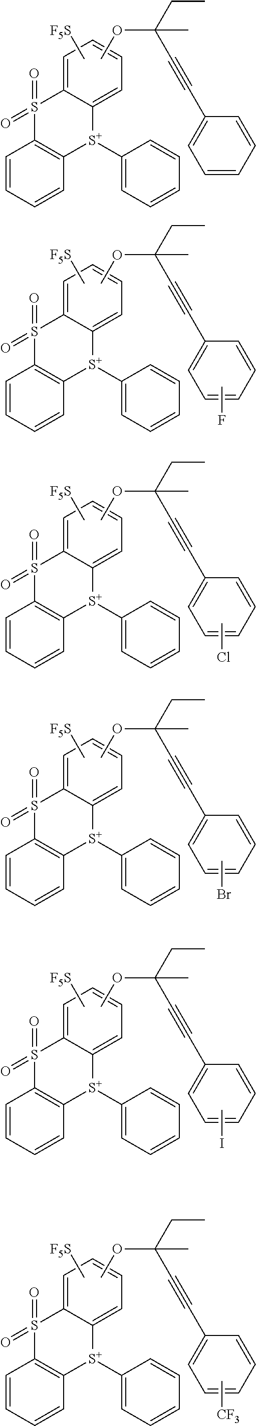

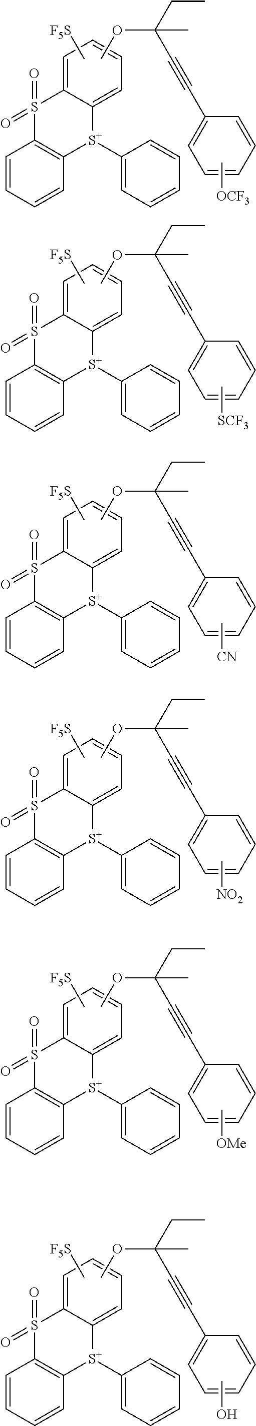

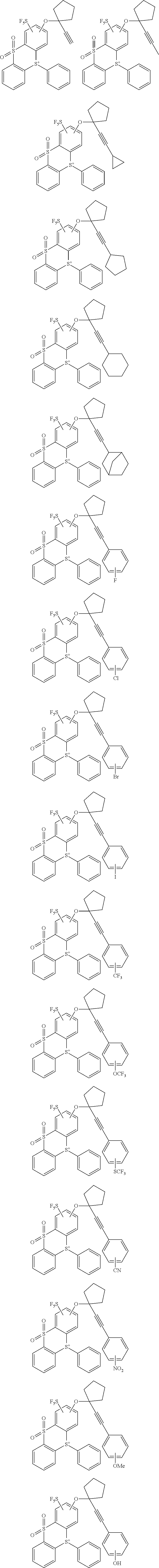

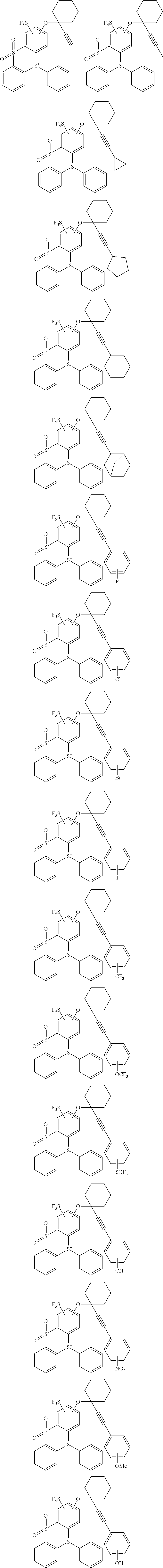

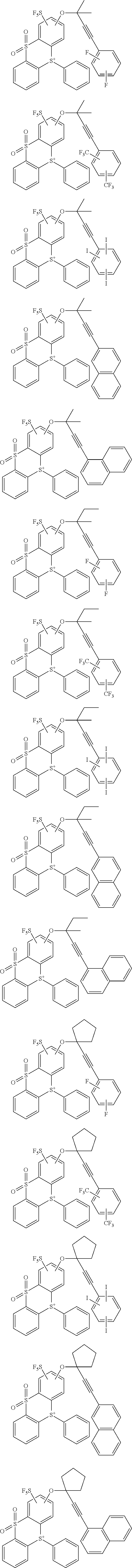

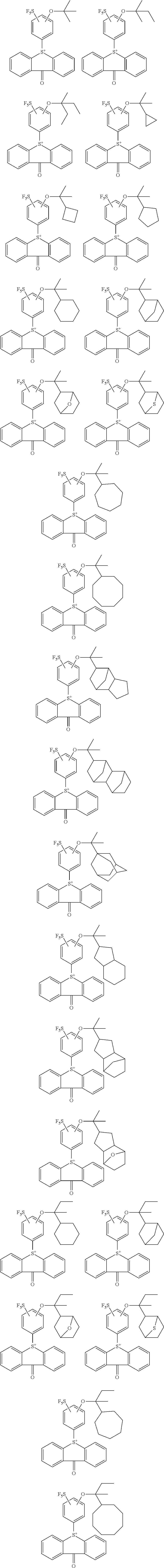

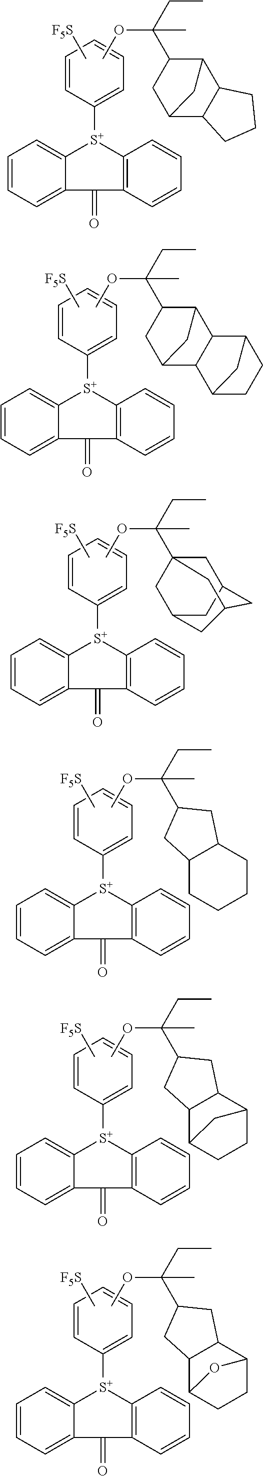

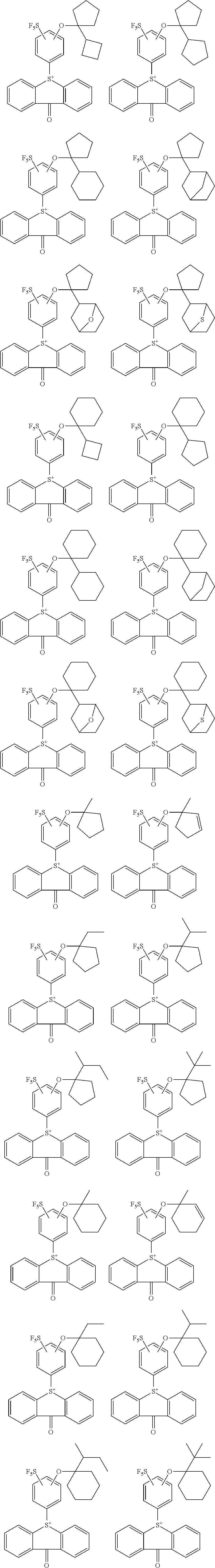

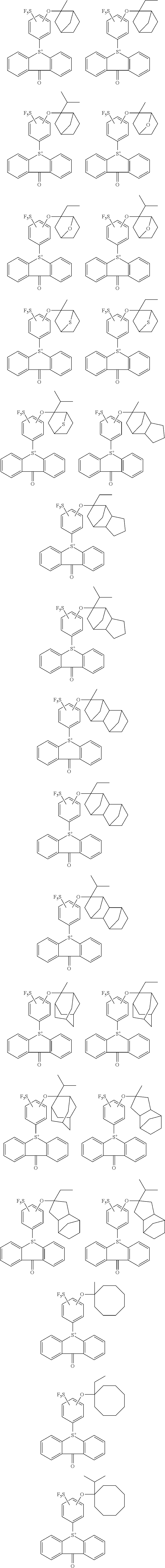

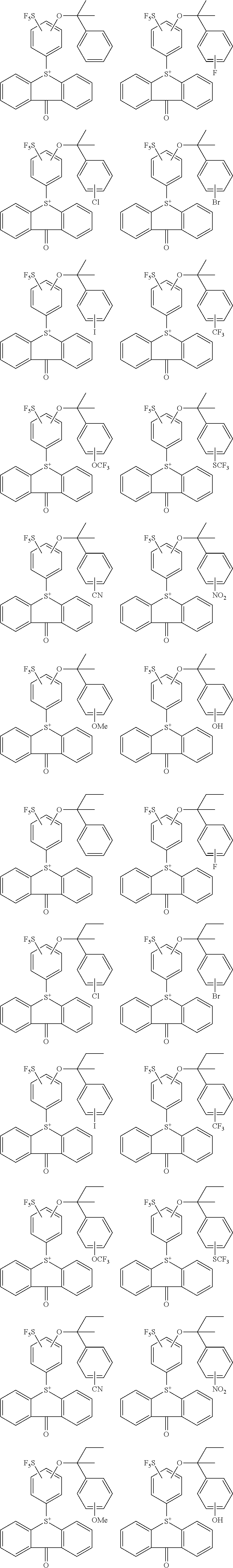

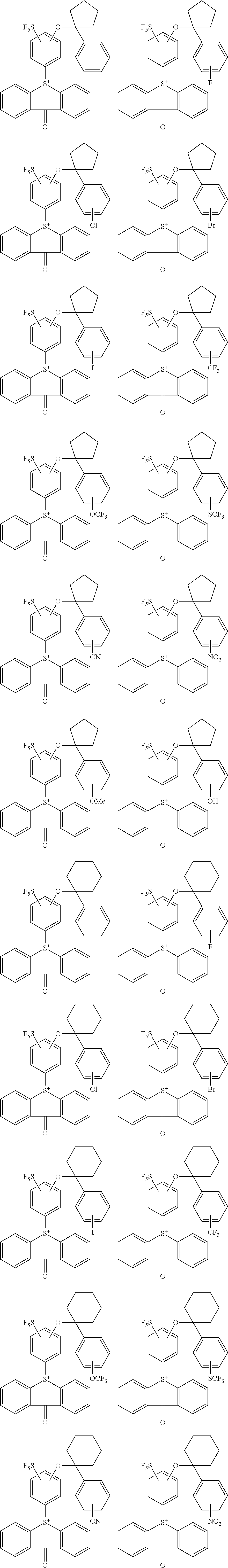

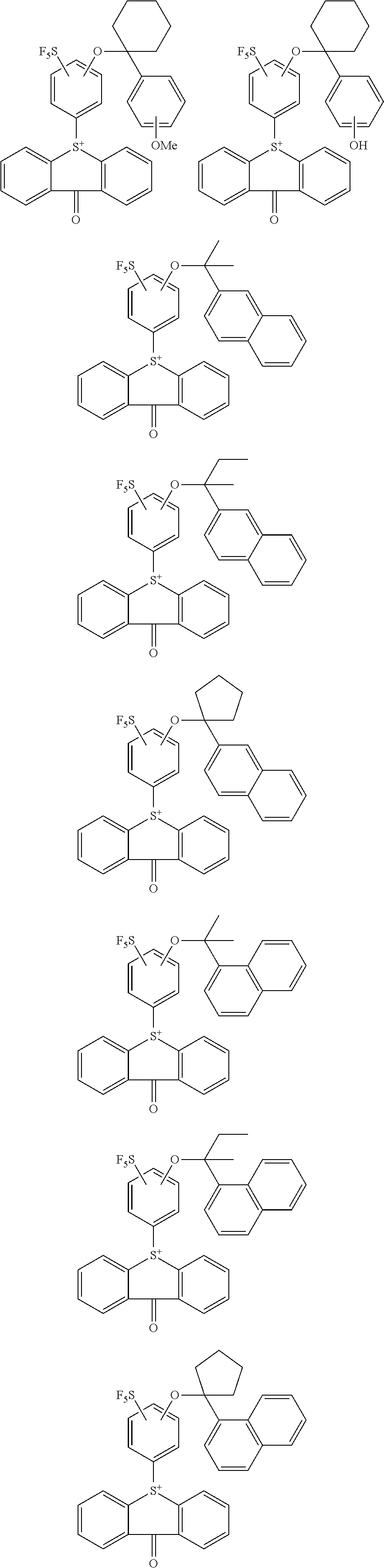

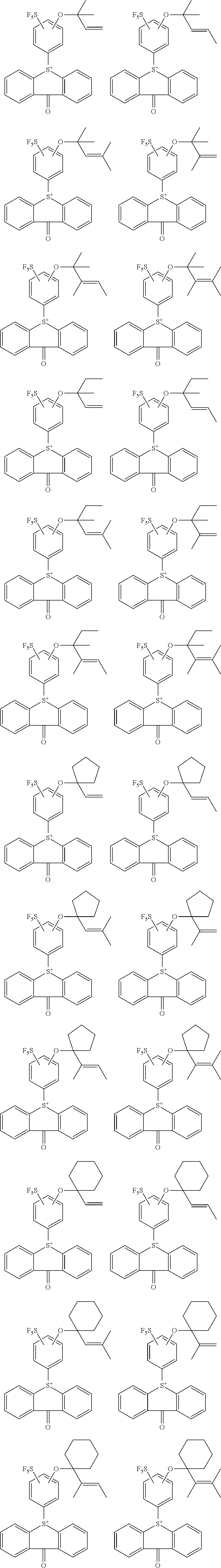

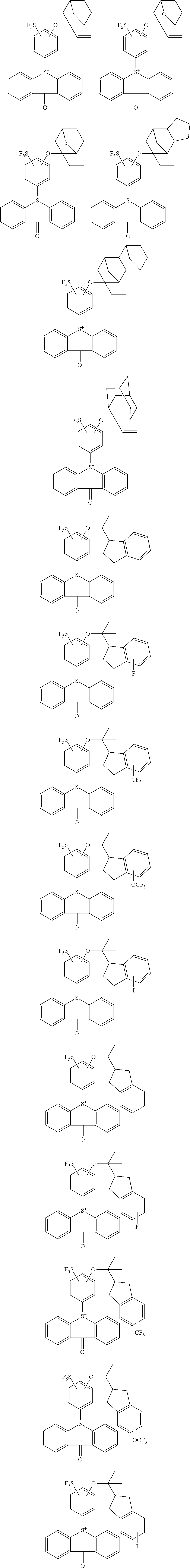

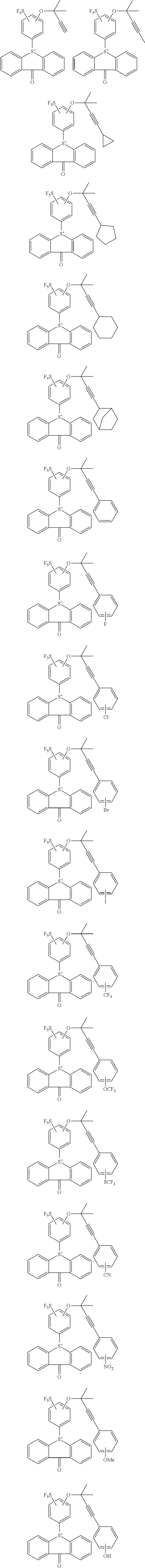

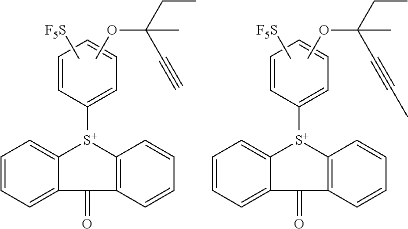

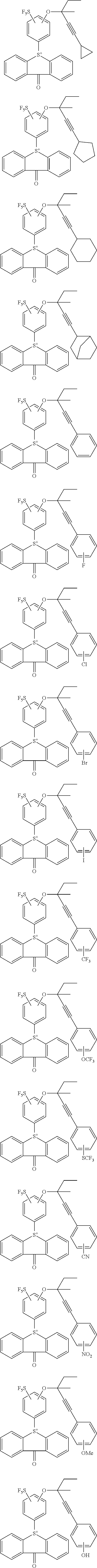

[chemical structure]

[chemical structure]

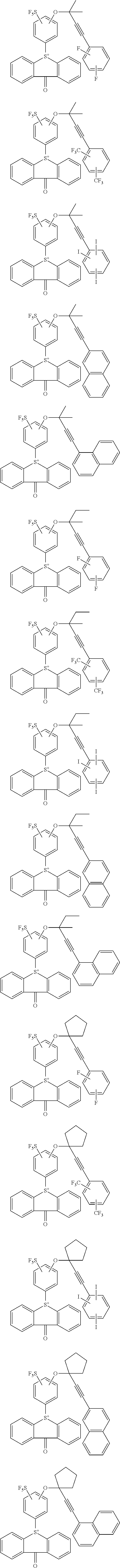

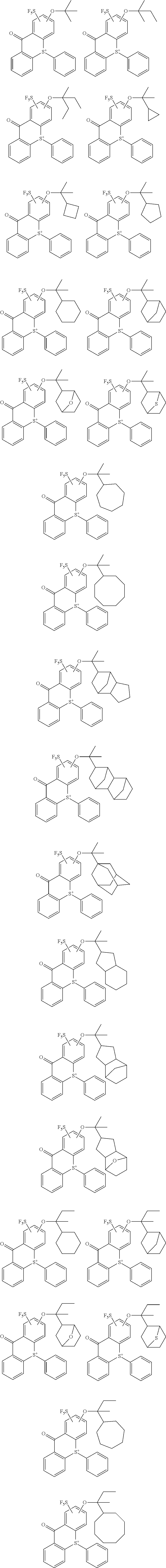

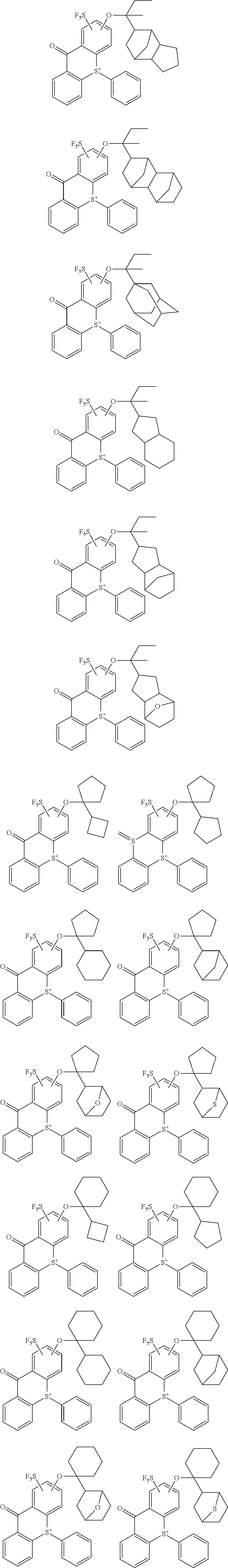

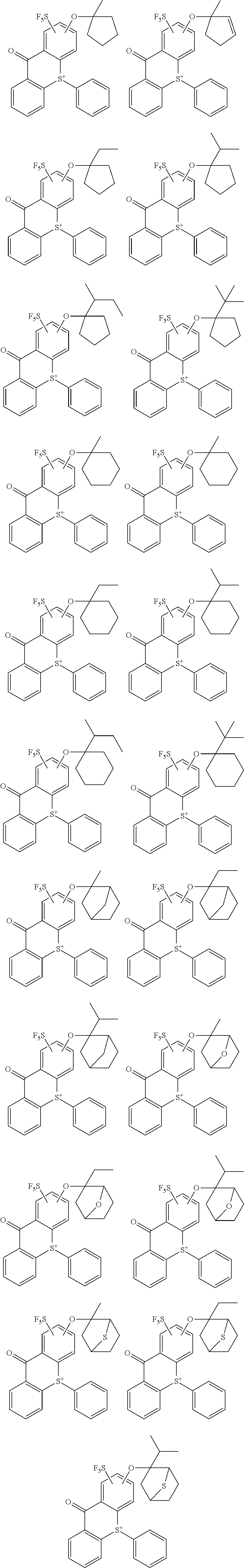

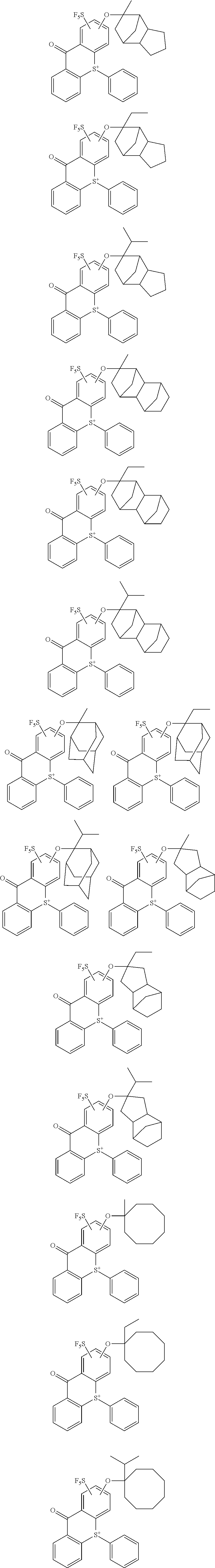

[chemical structure]

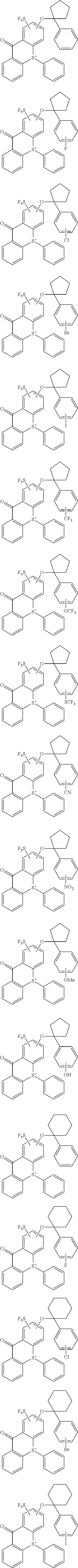

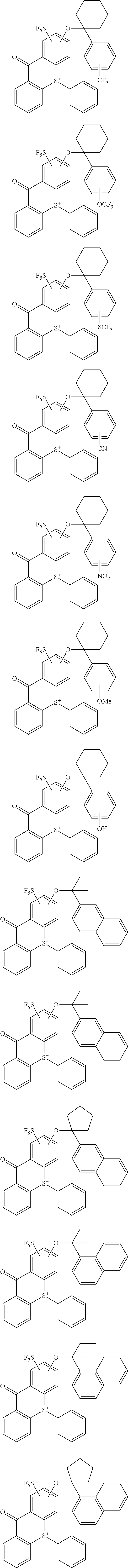

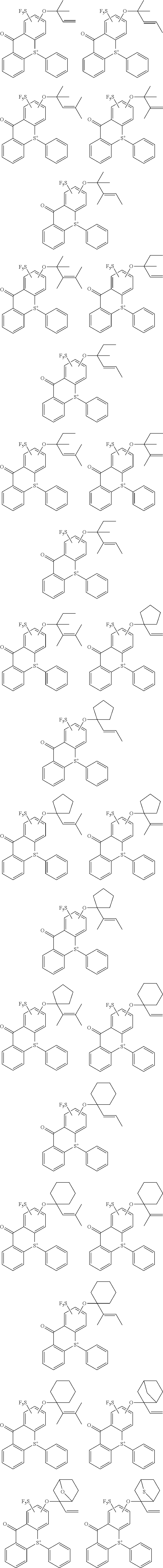

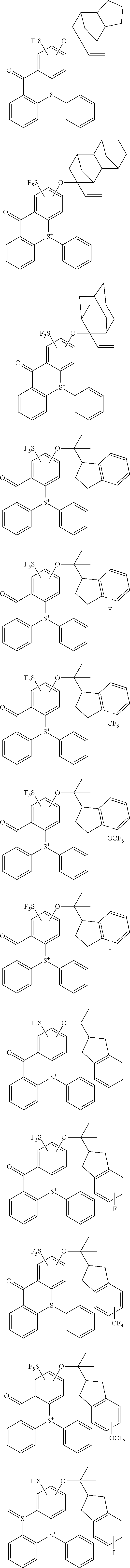

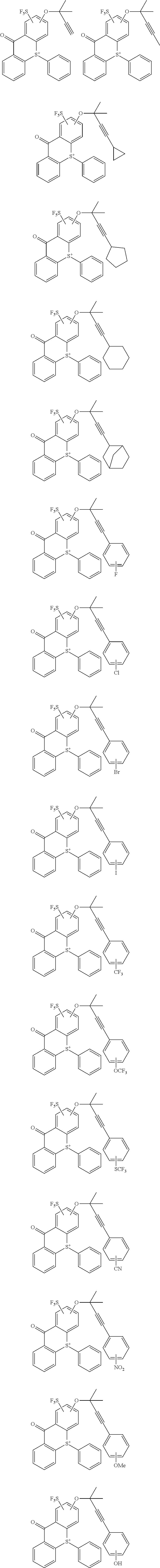

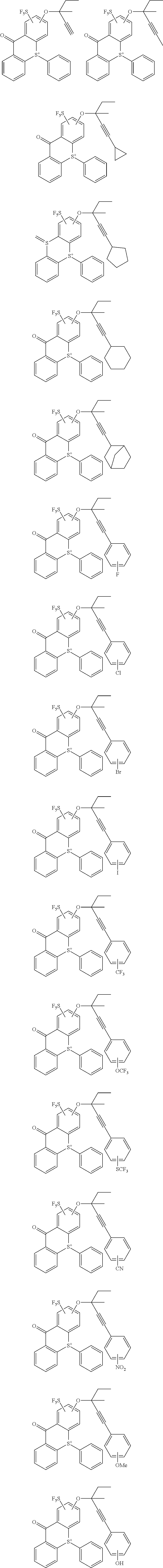

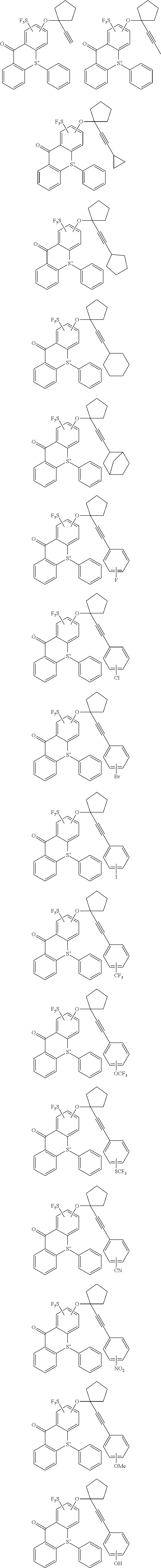

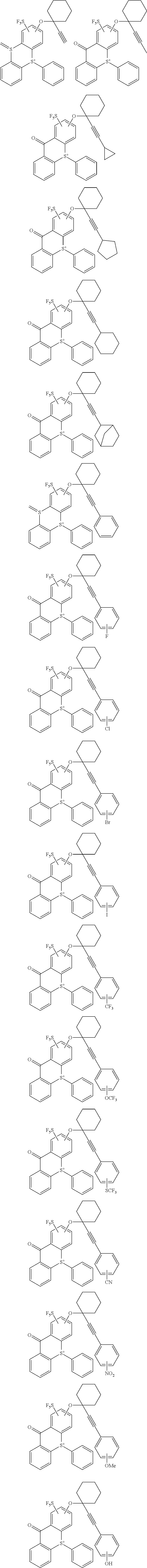

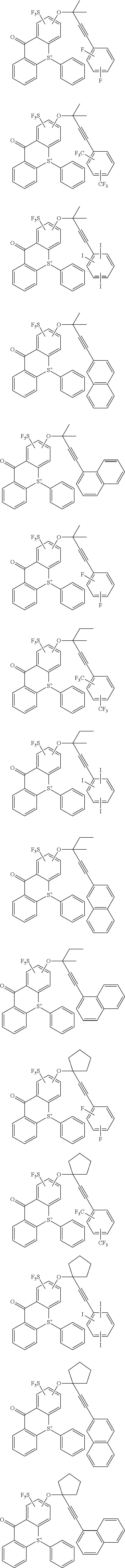

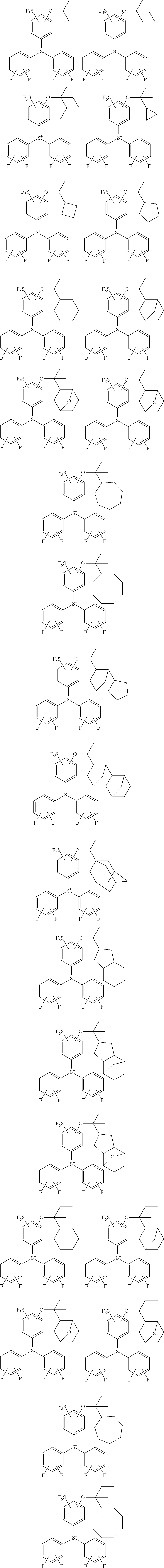

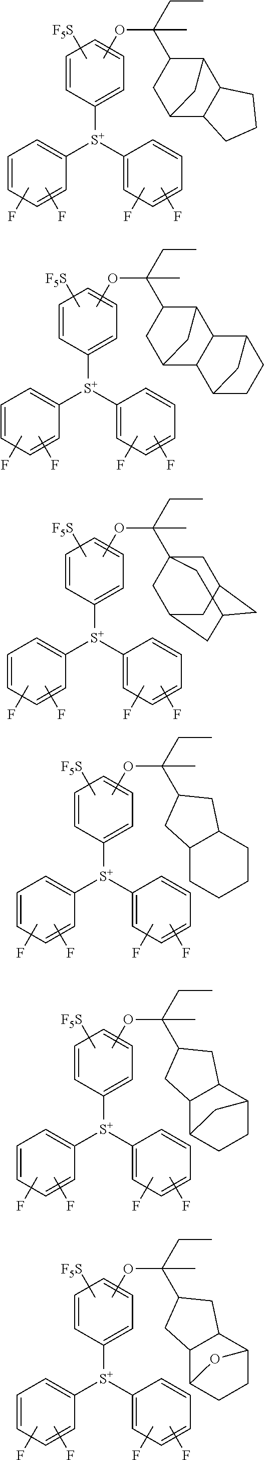

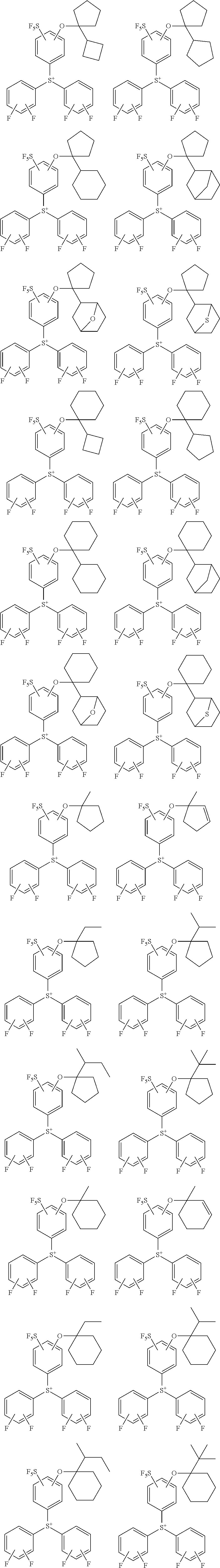

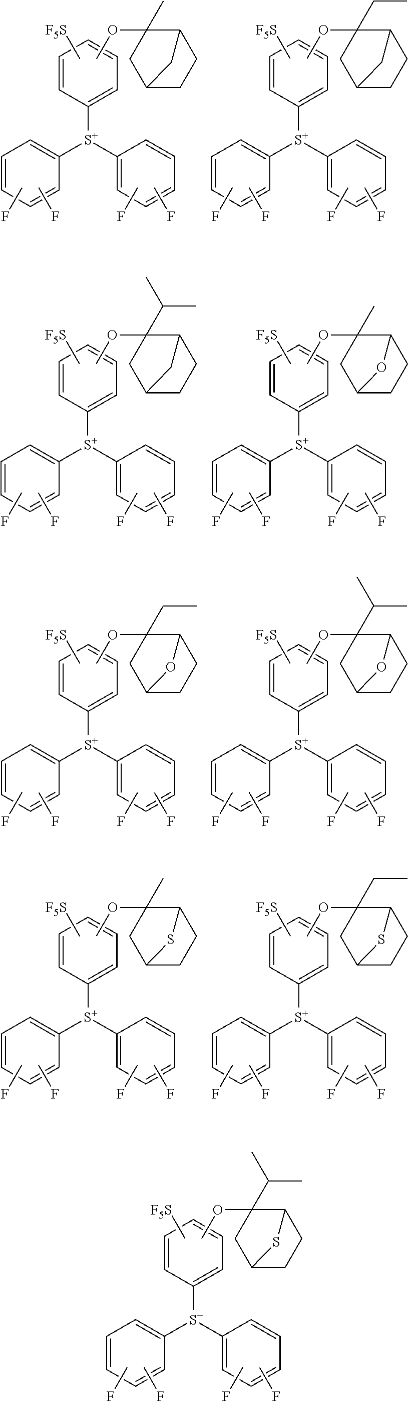

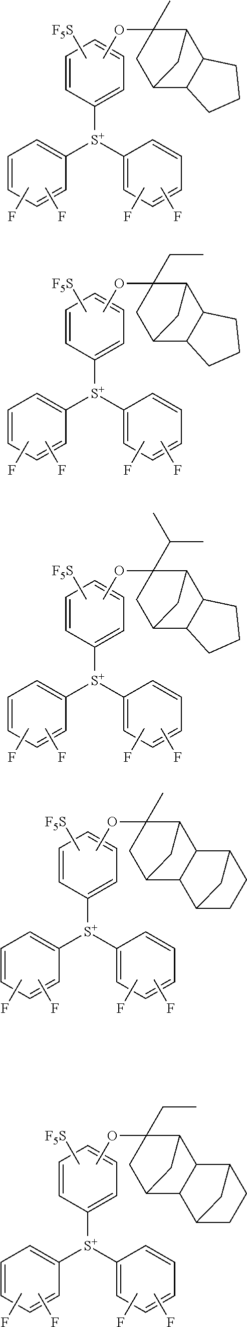

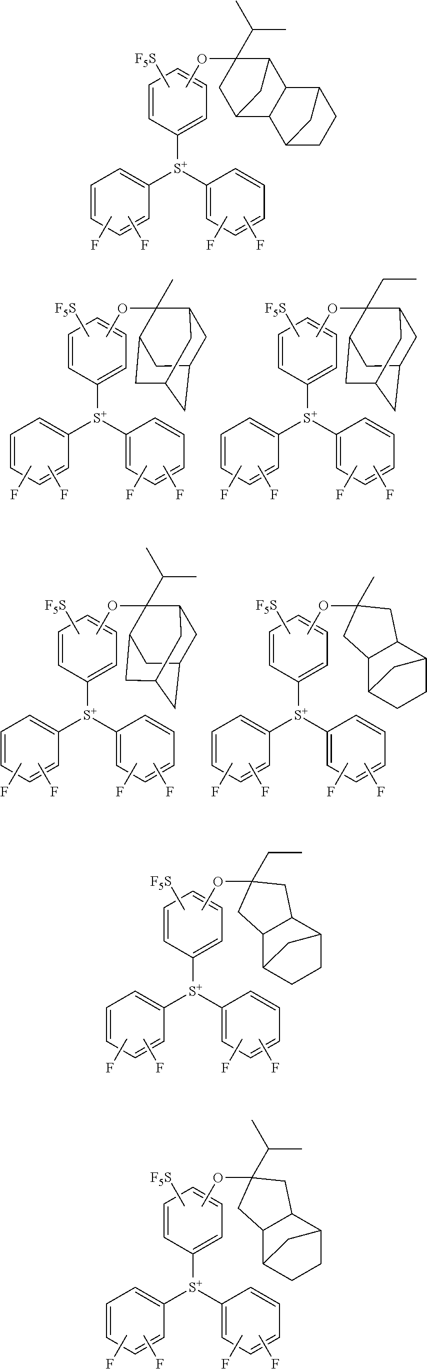

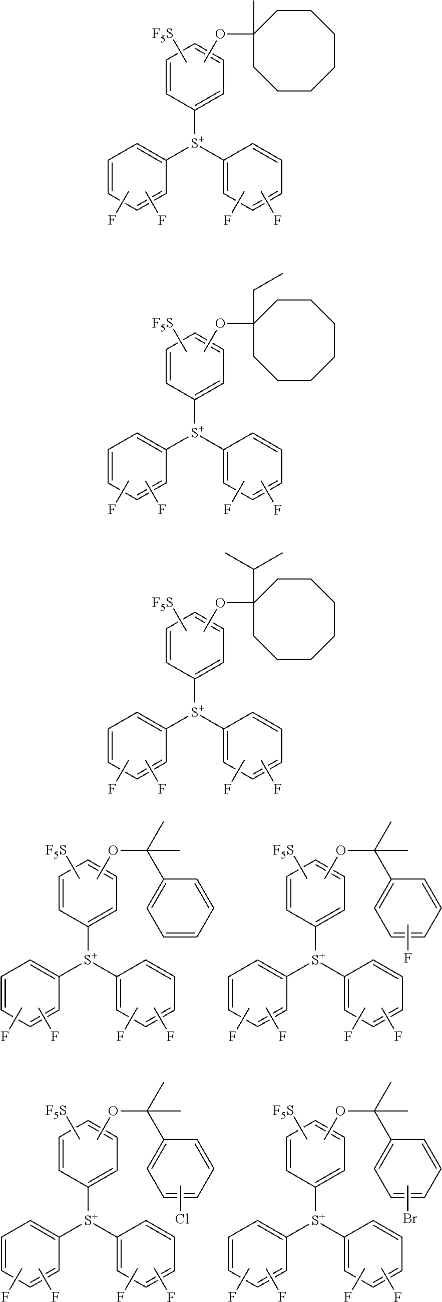

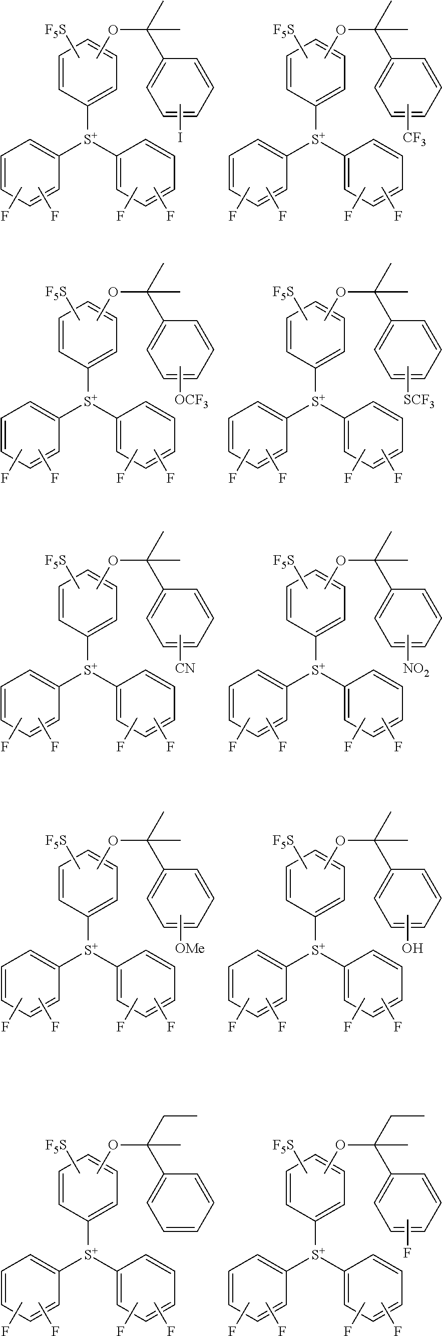

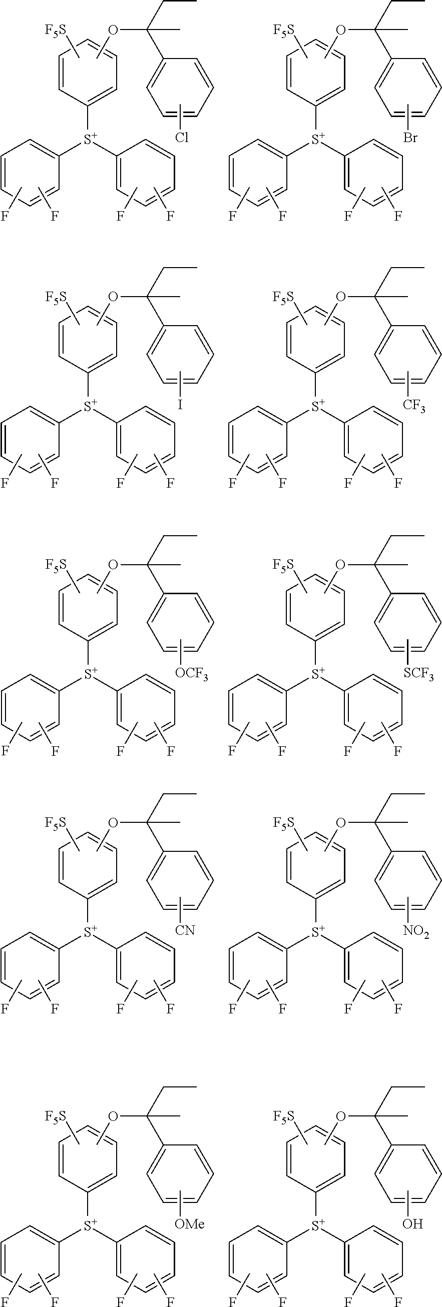

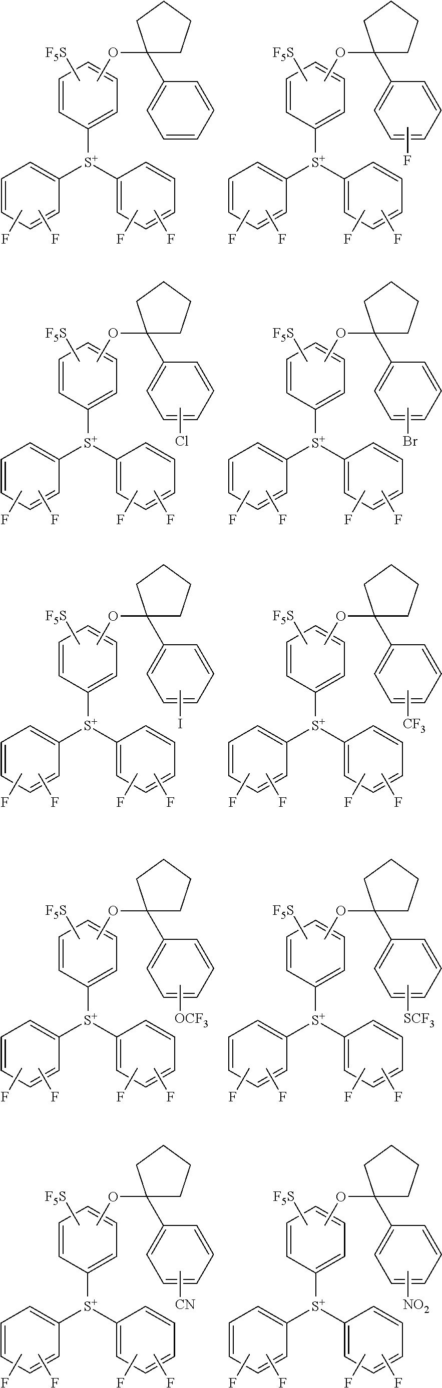

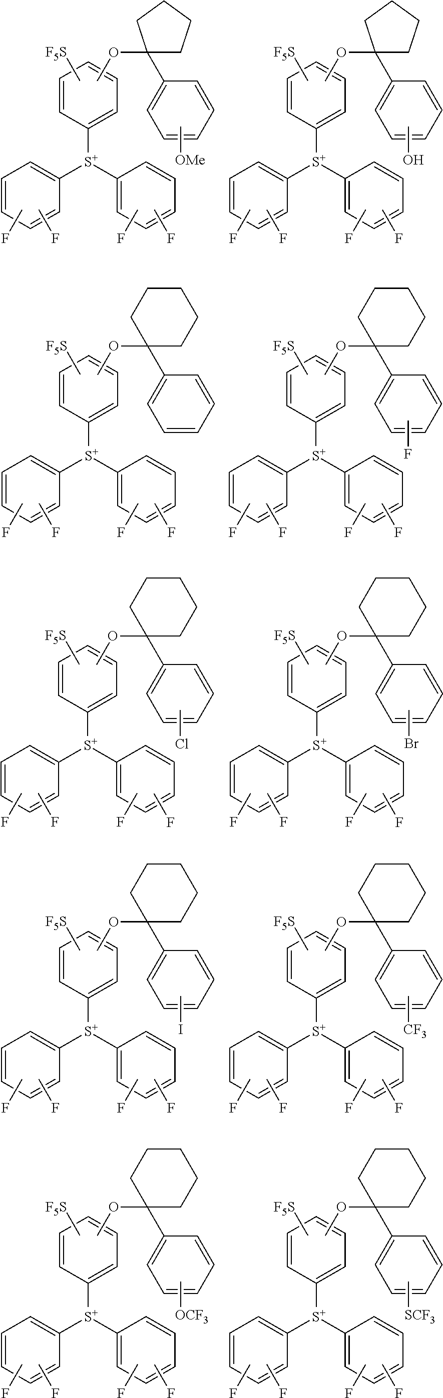

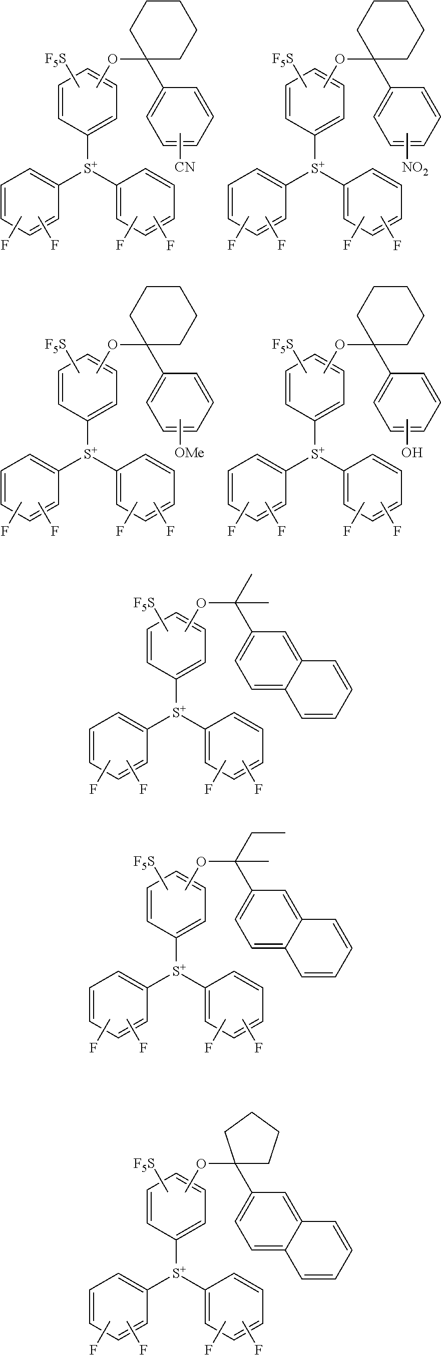

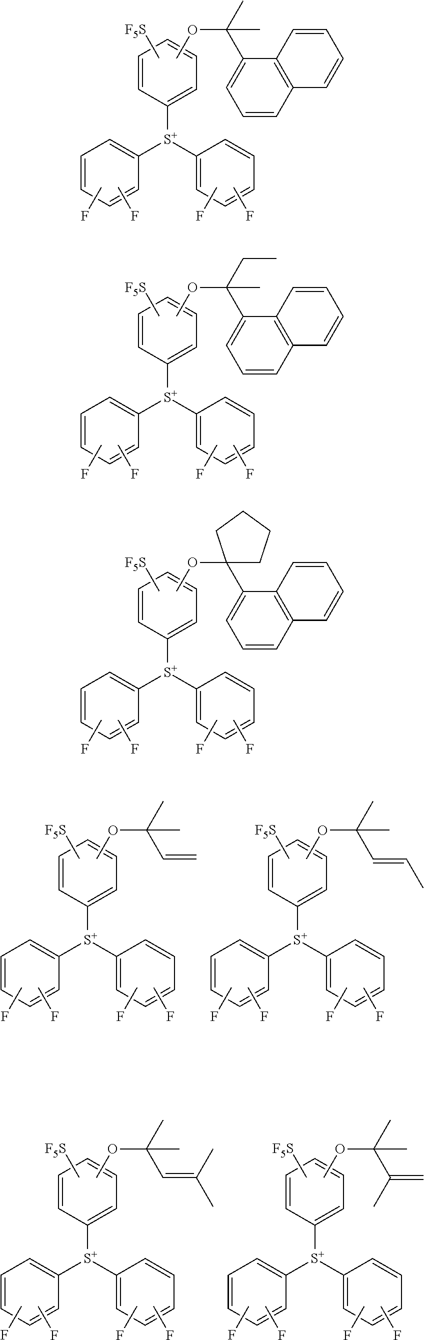

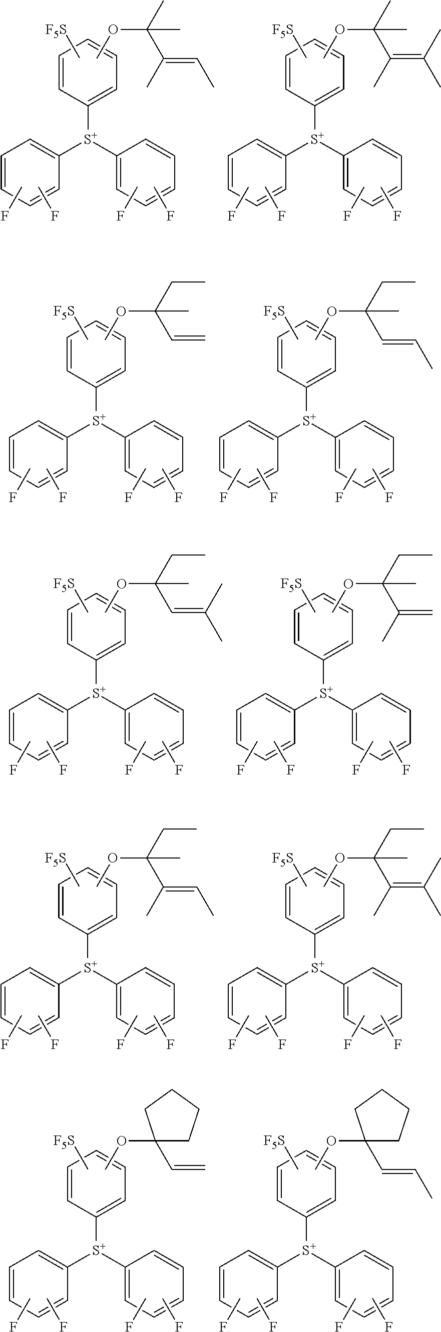

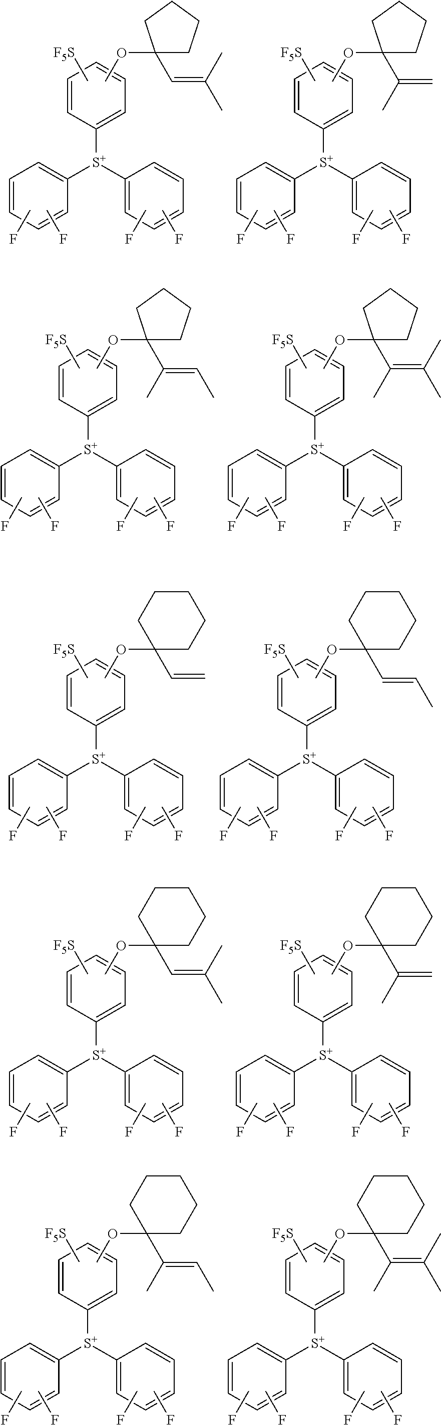

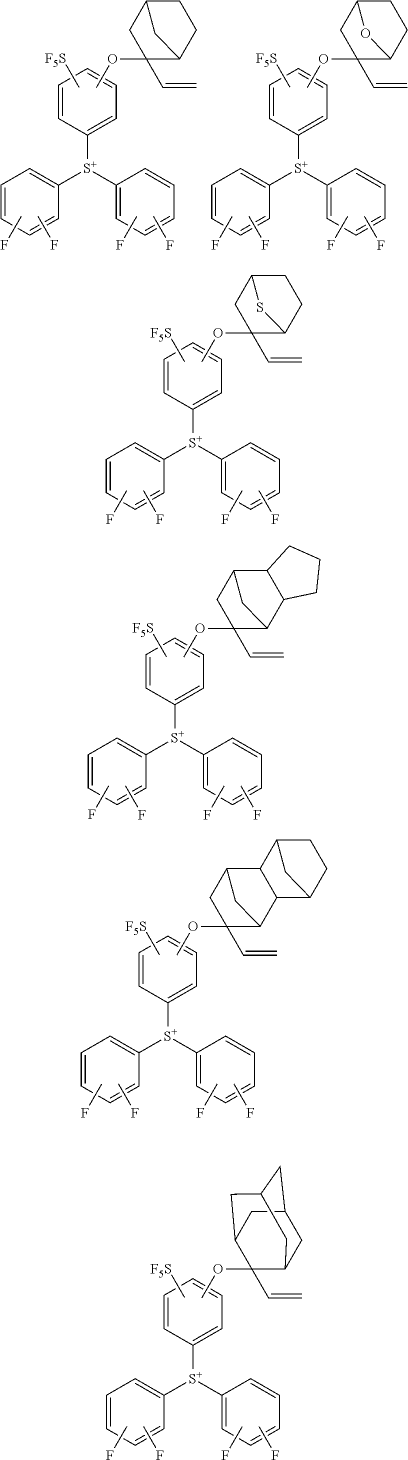

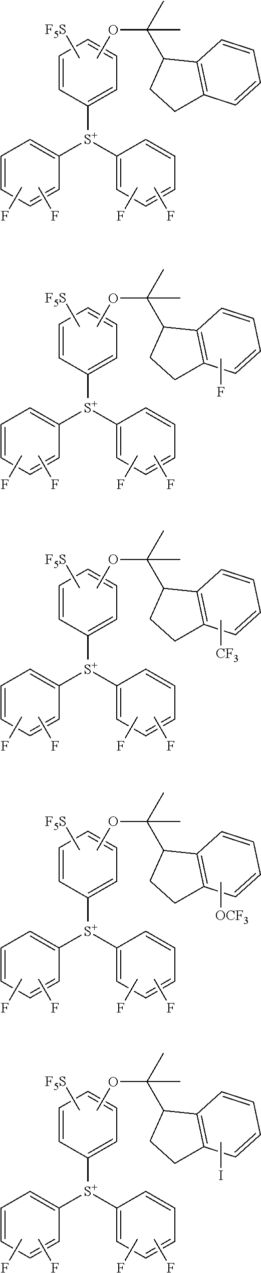

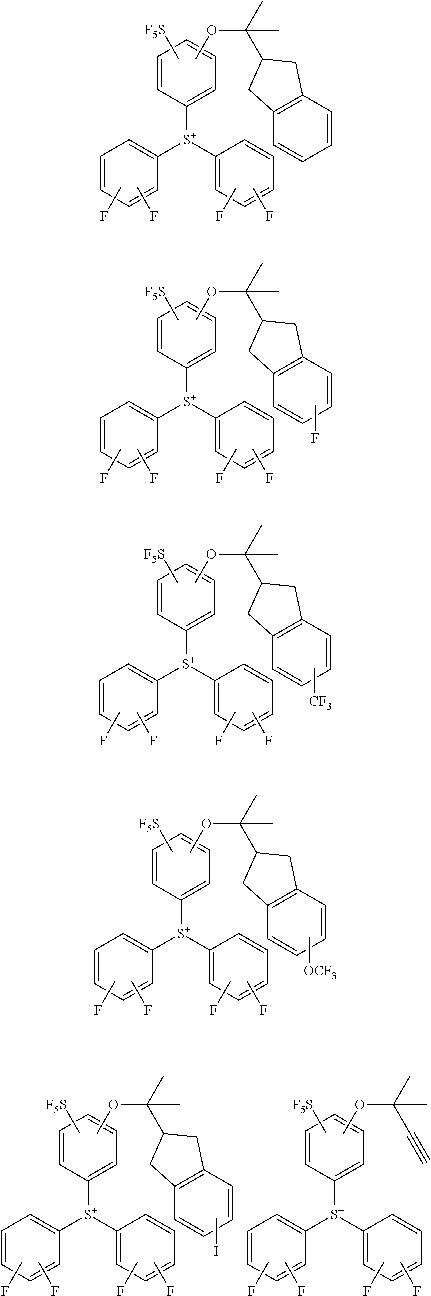

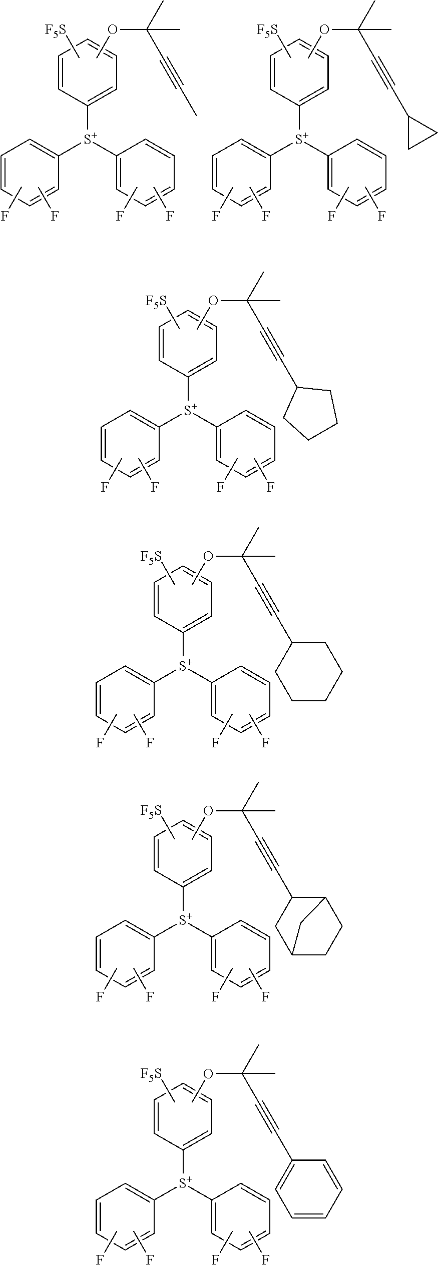

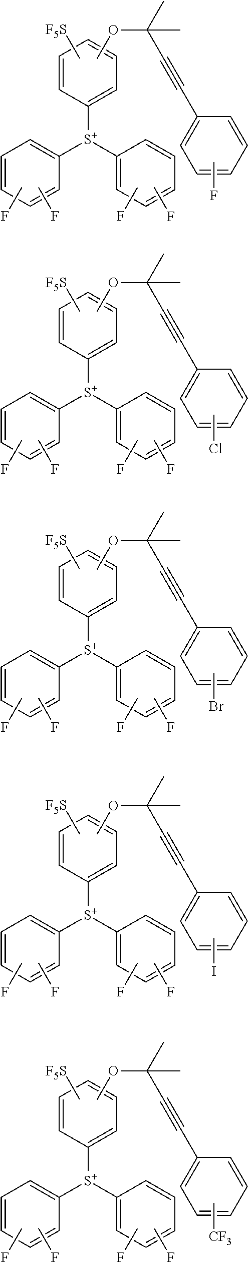

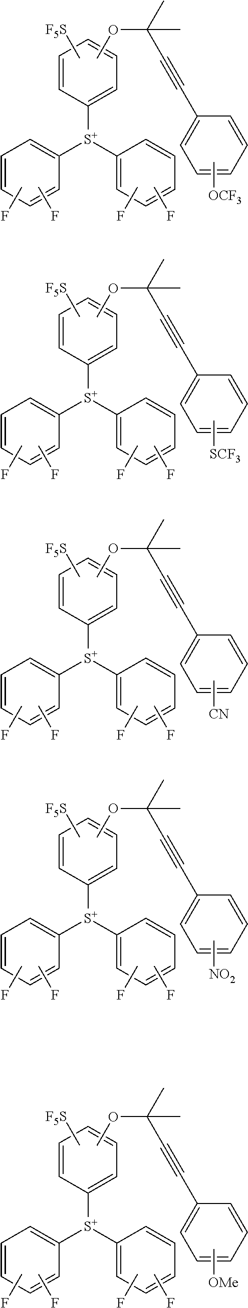

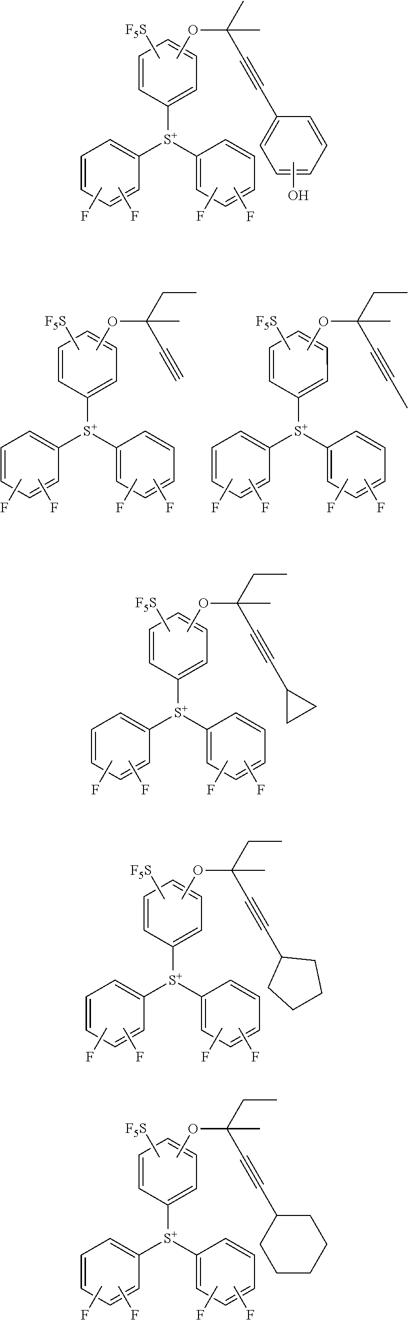

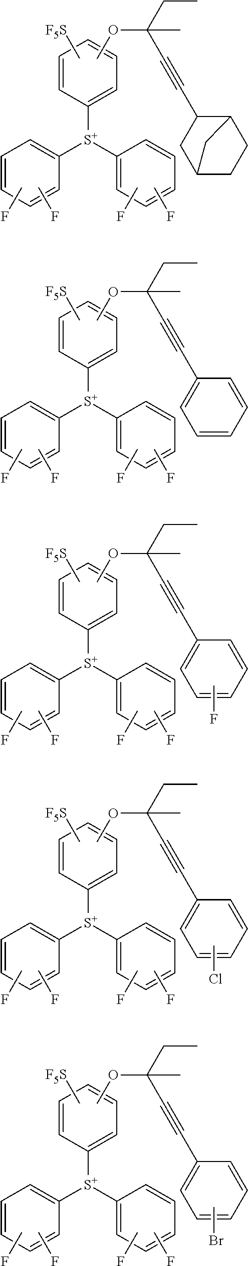

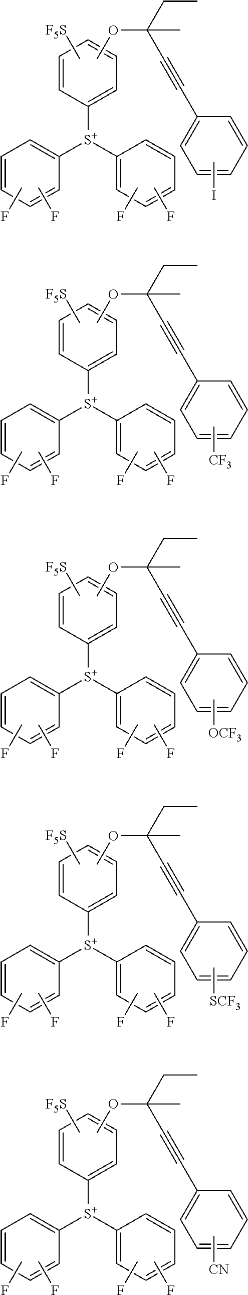

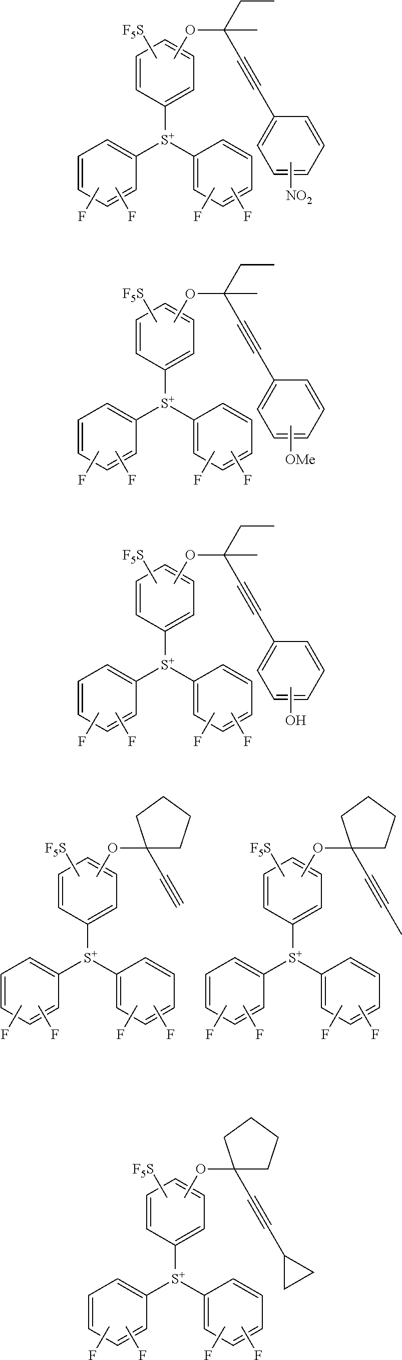

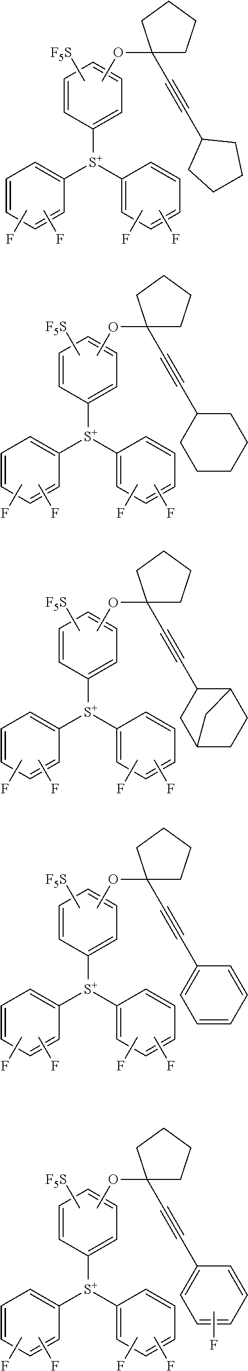

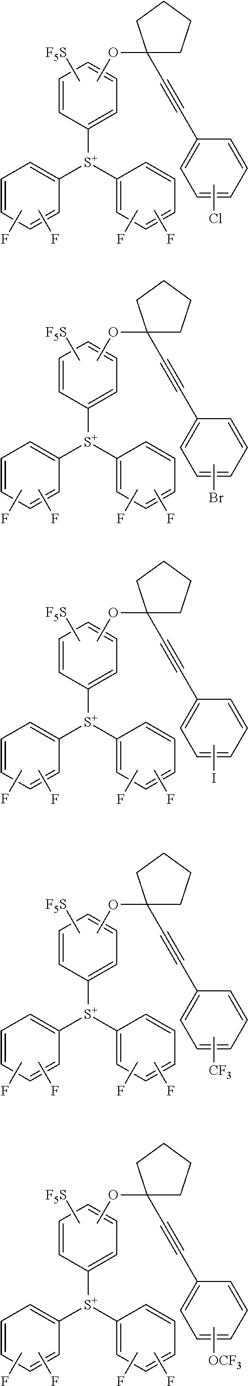

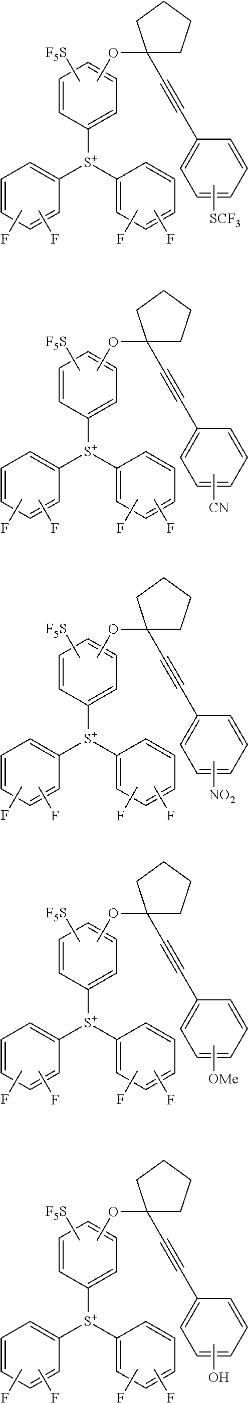

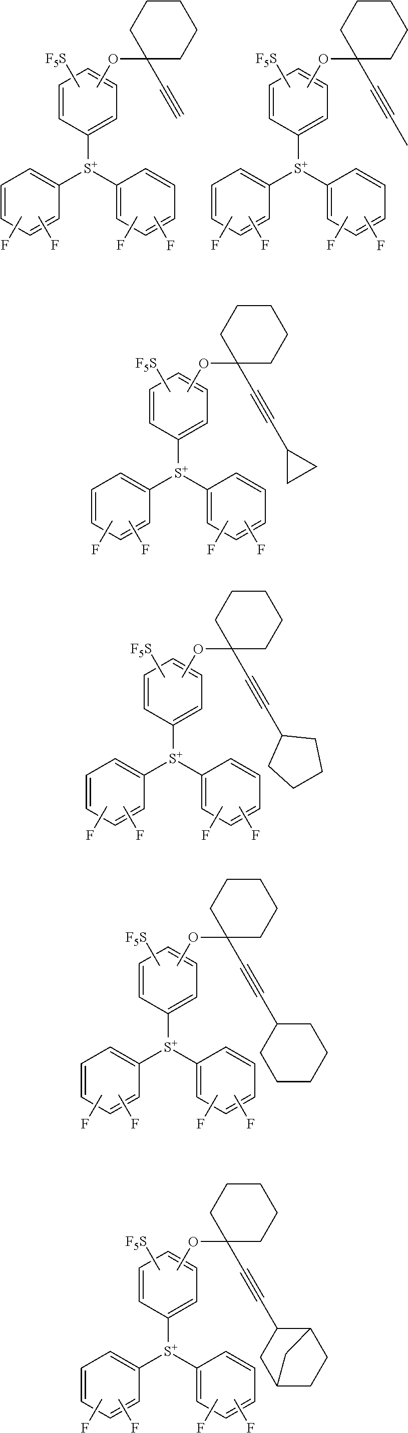

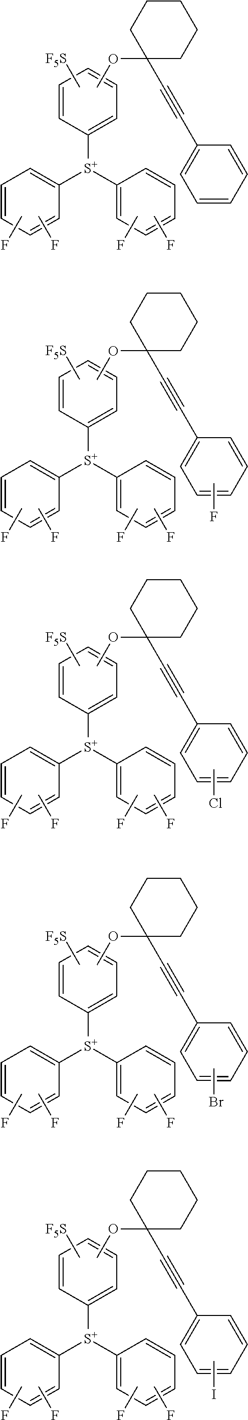

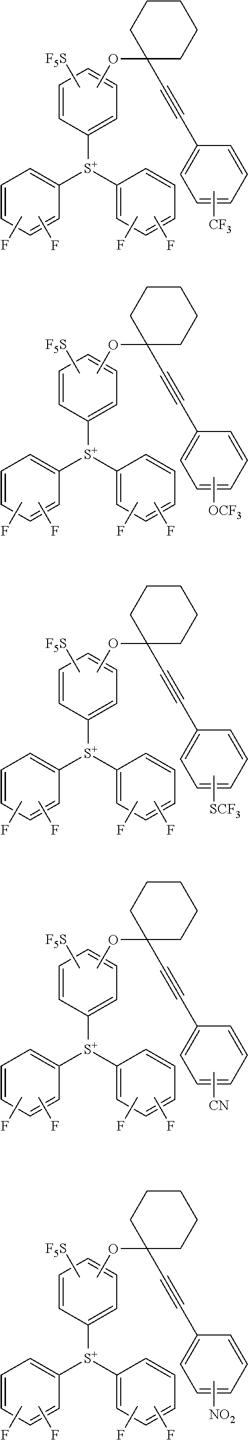

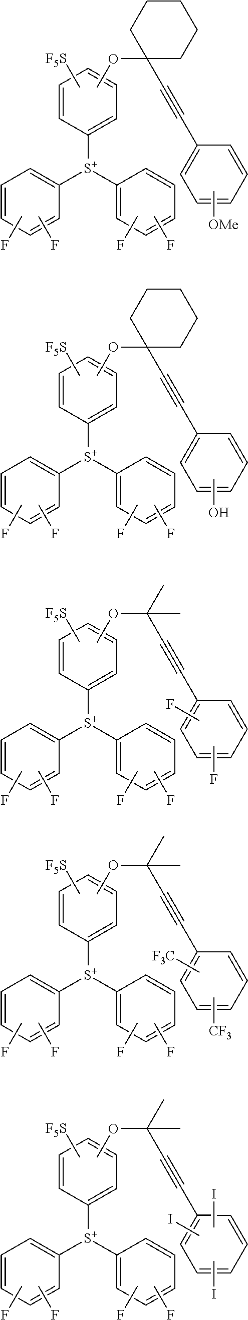

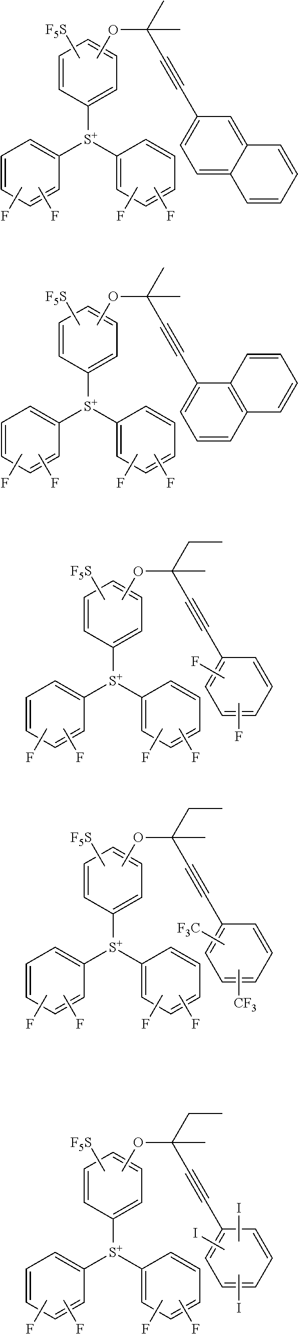

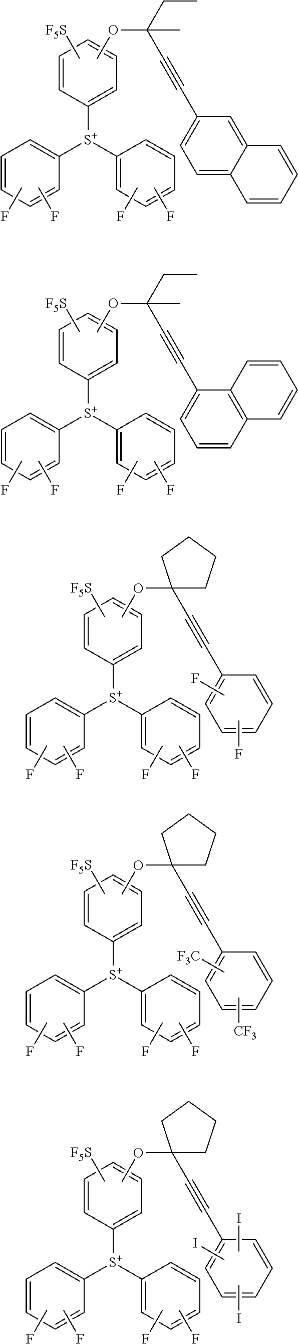

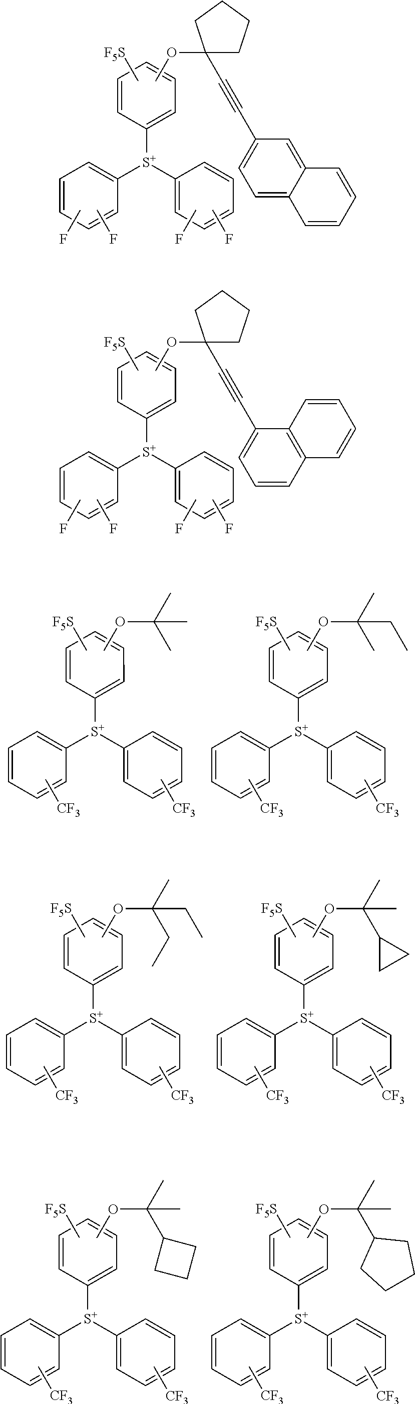

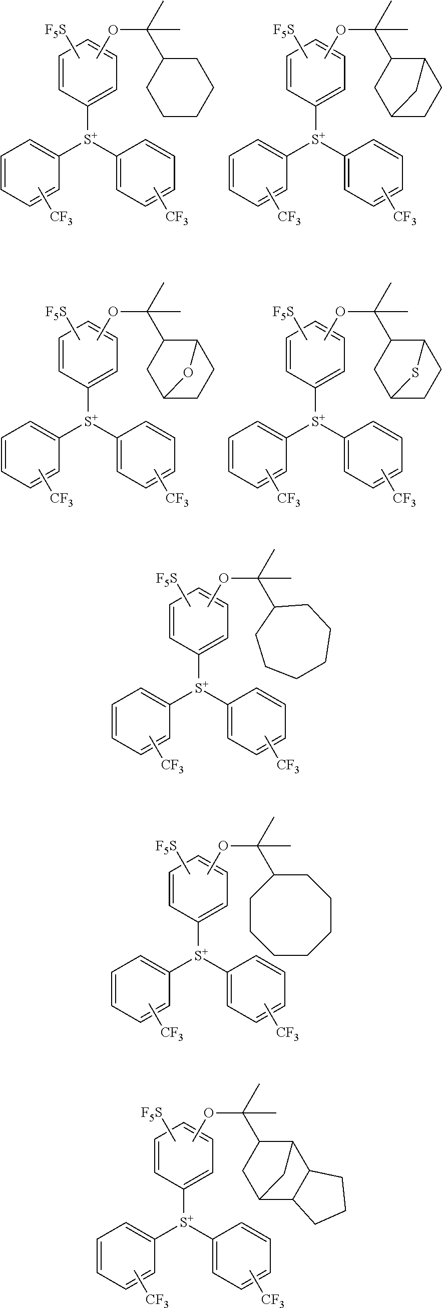

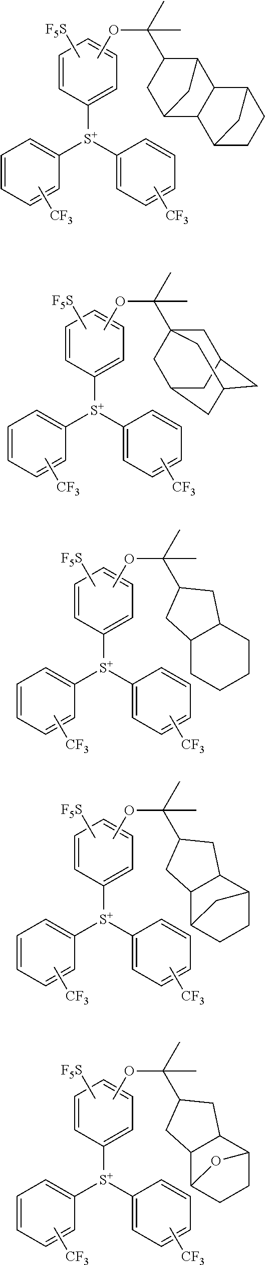

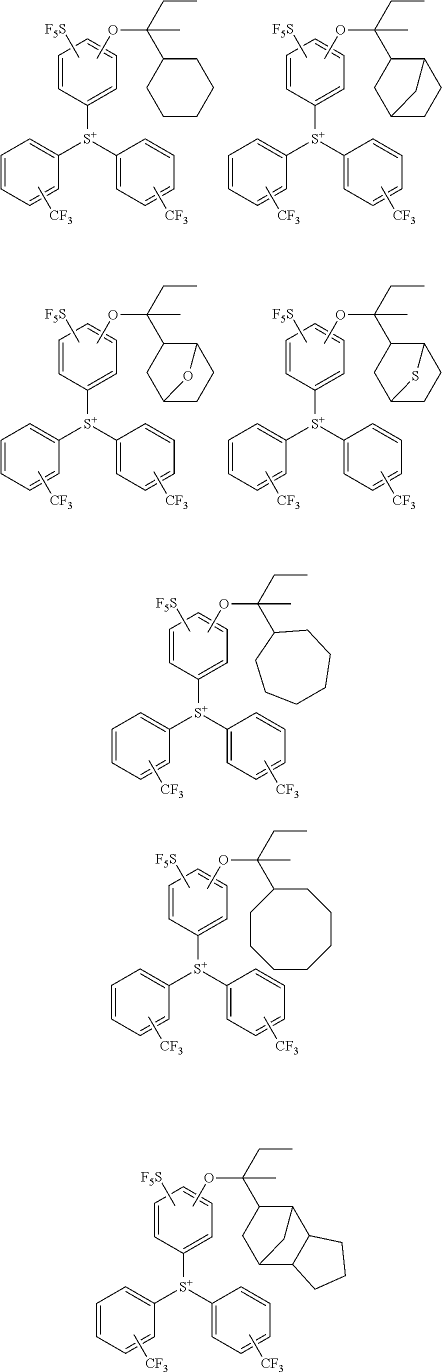

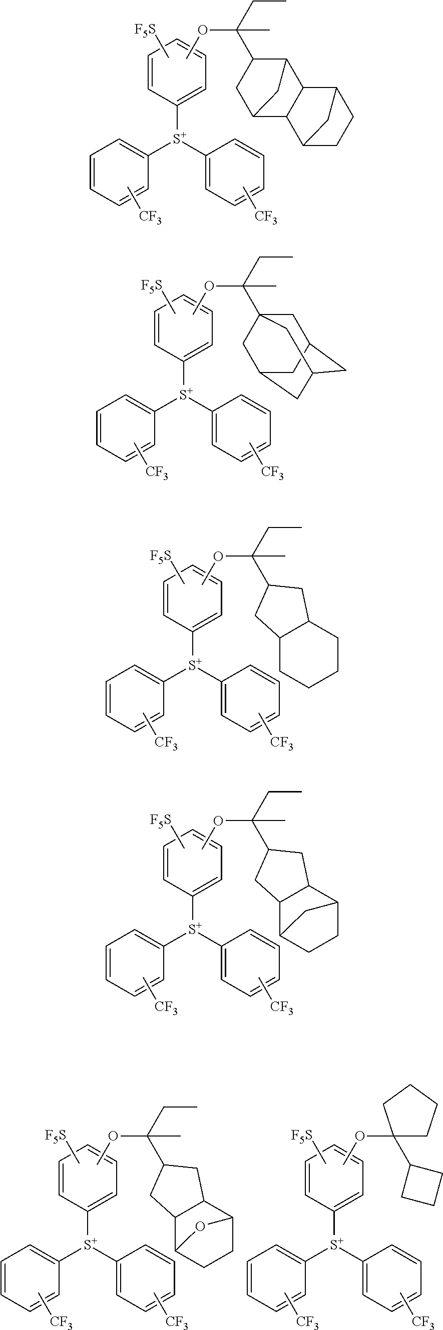

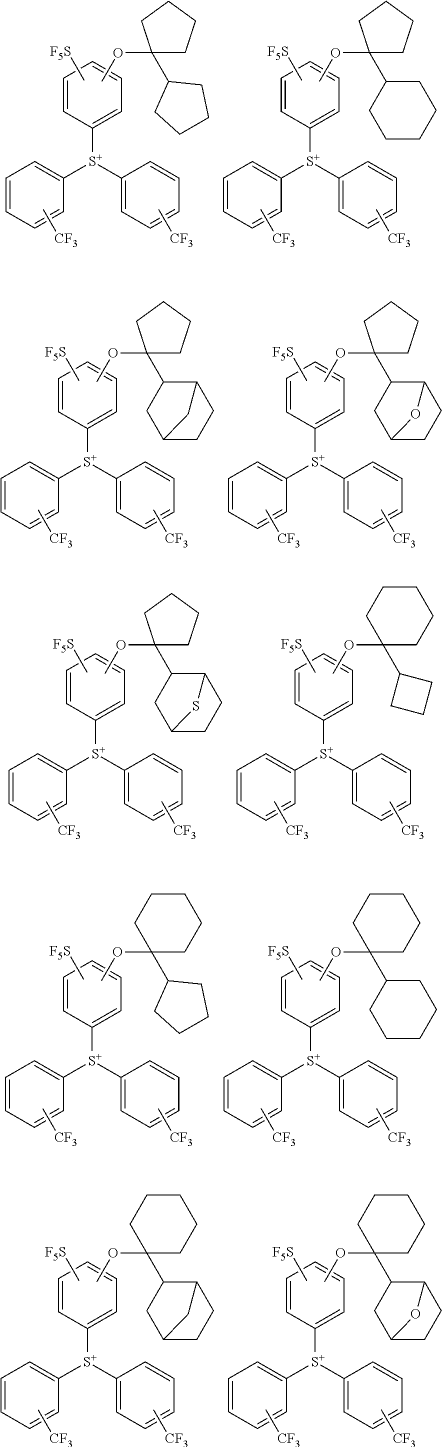

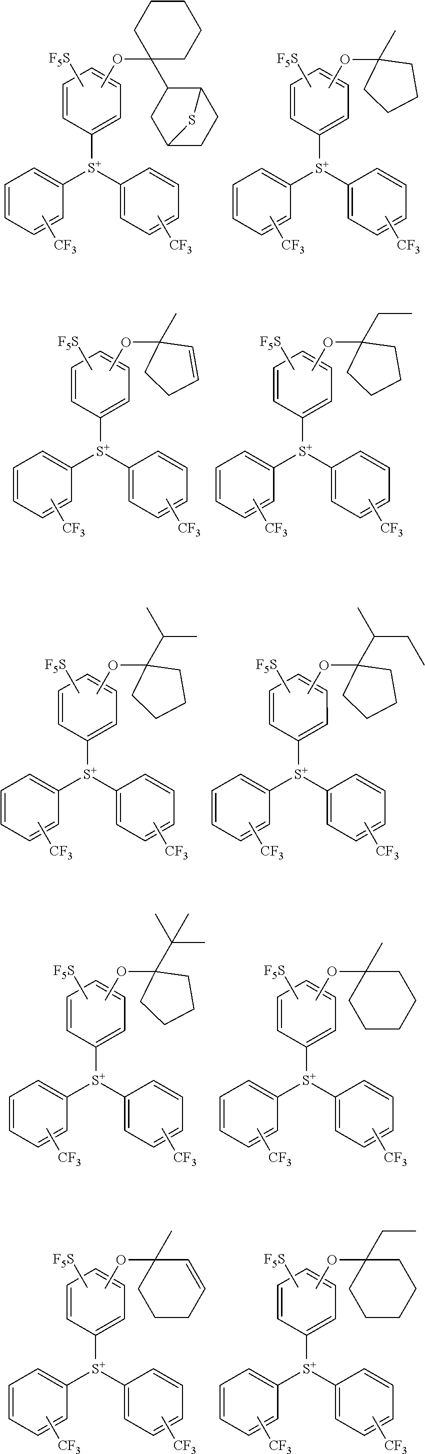

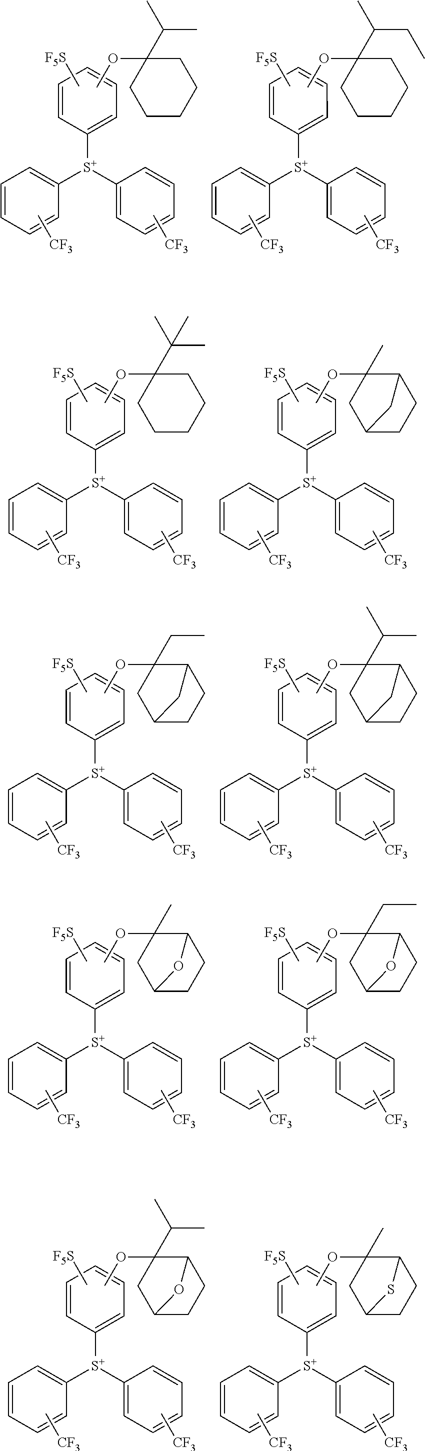

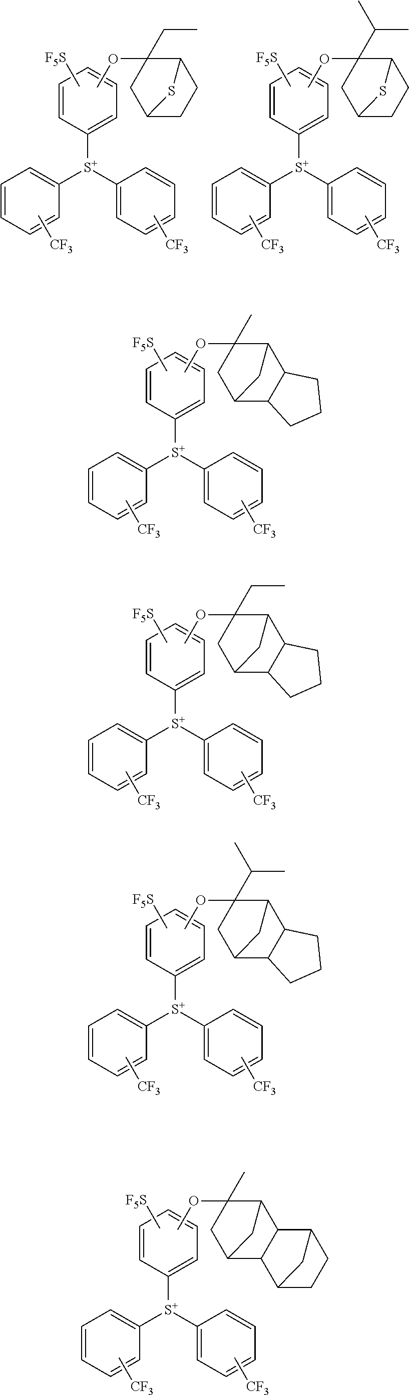

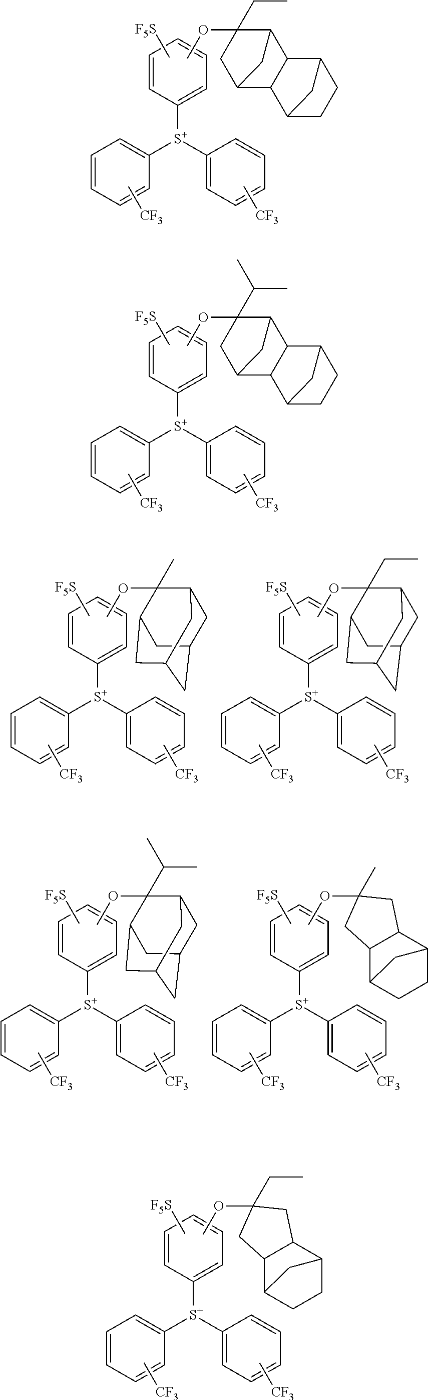

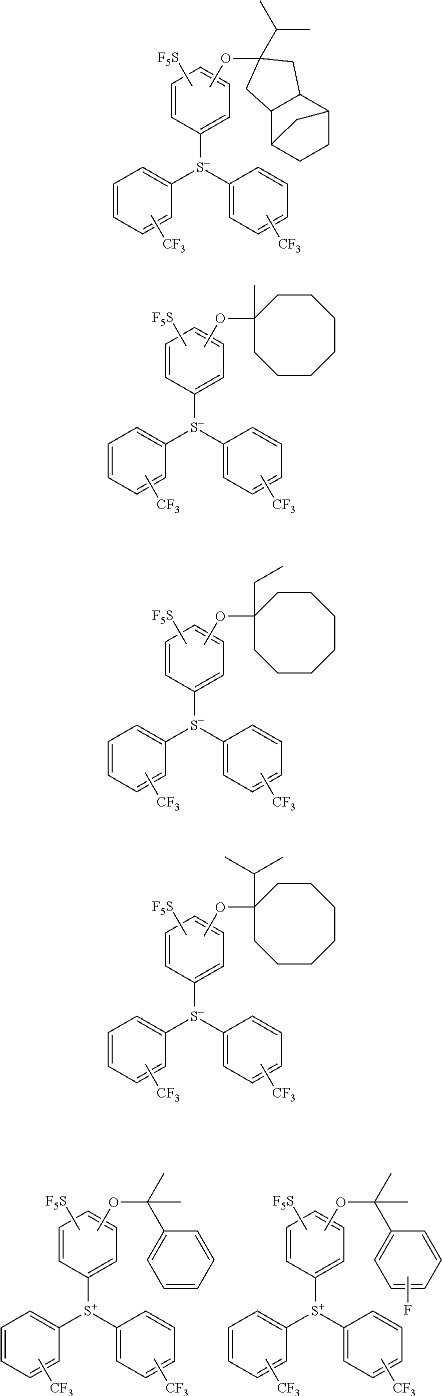

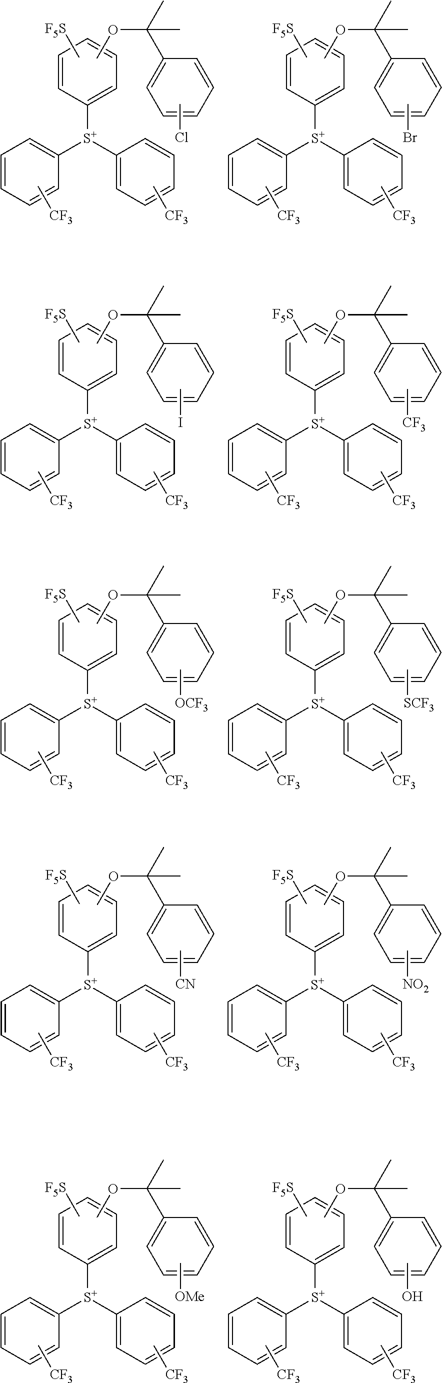

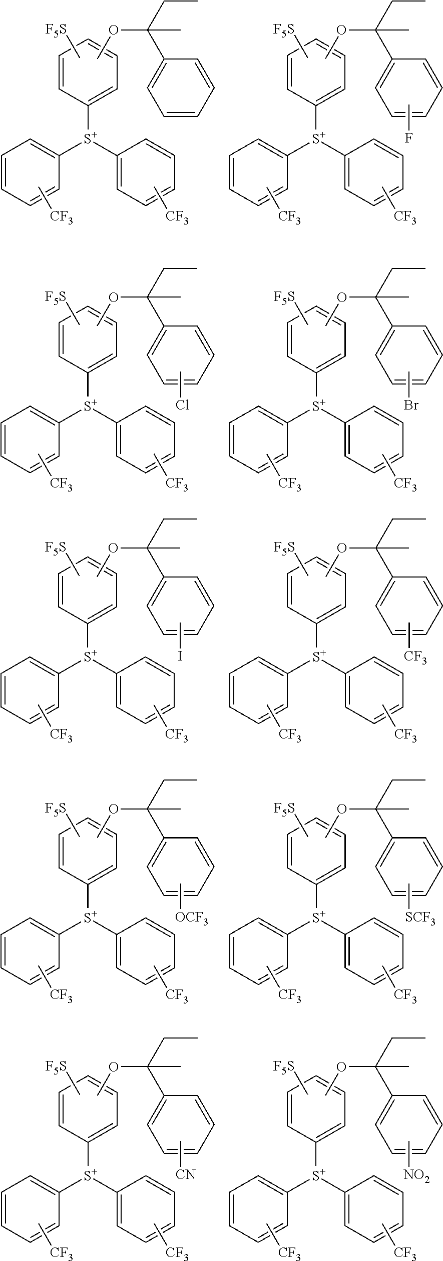

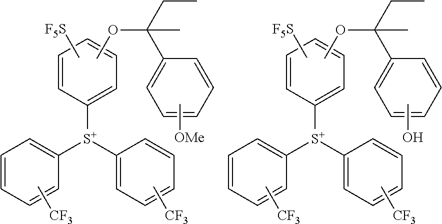

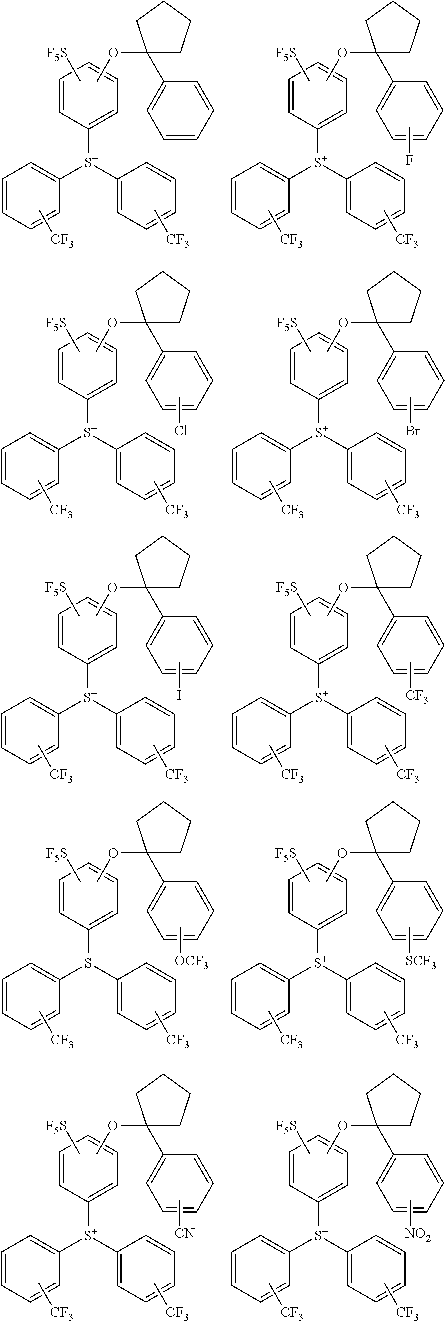

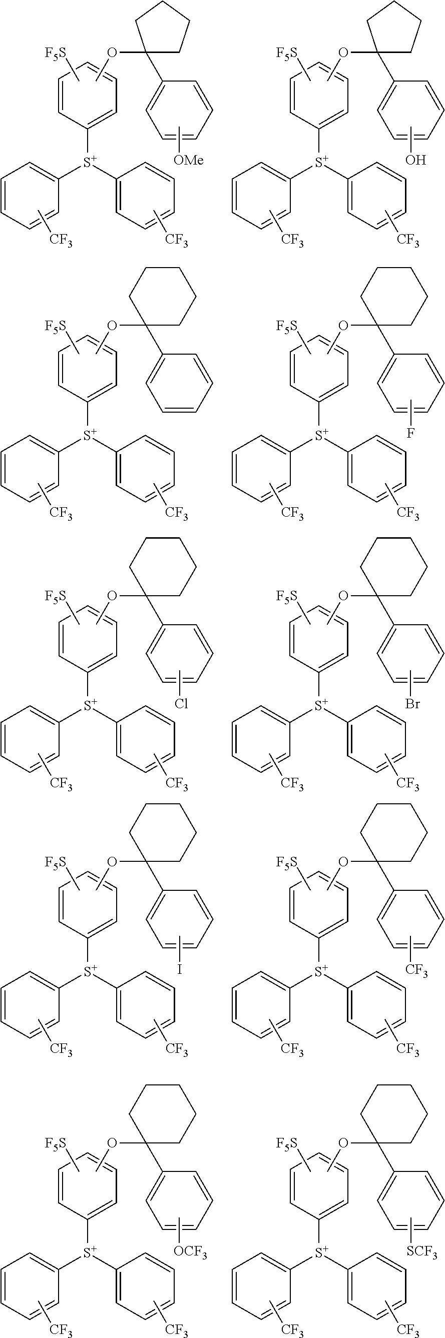

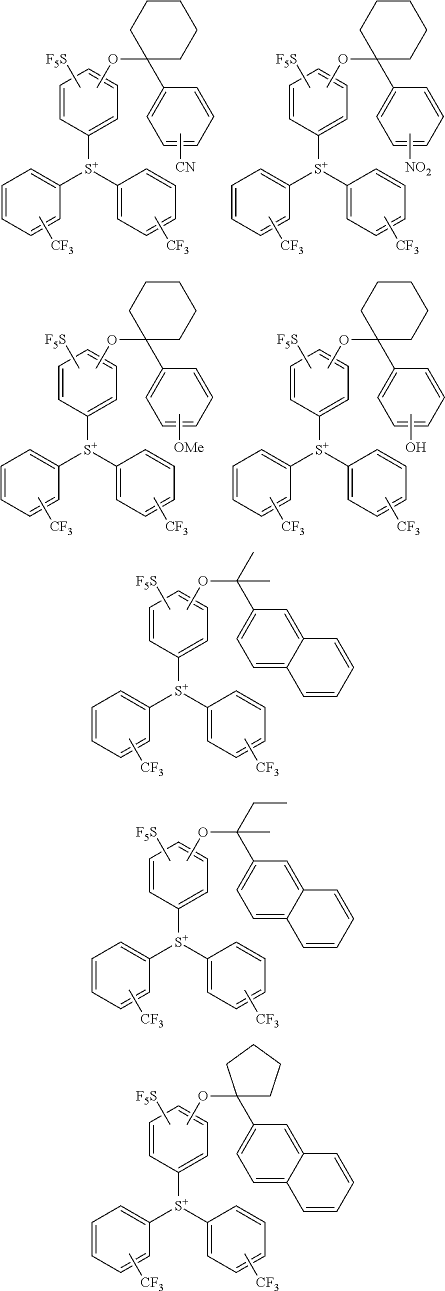

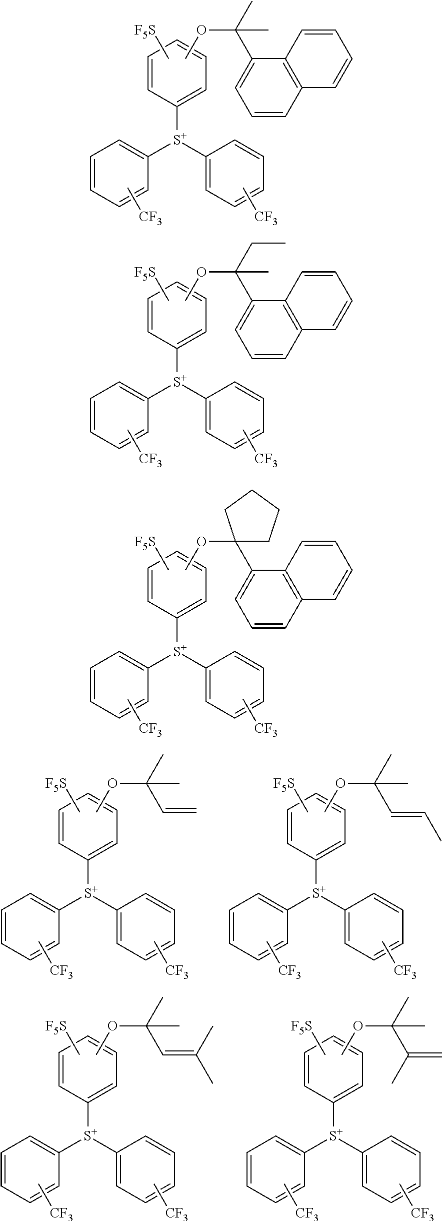

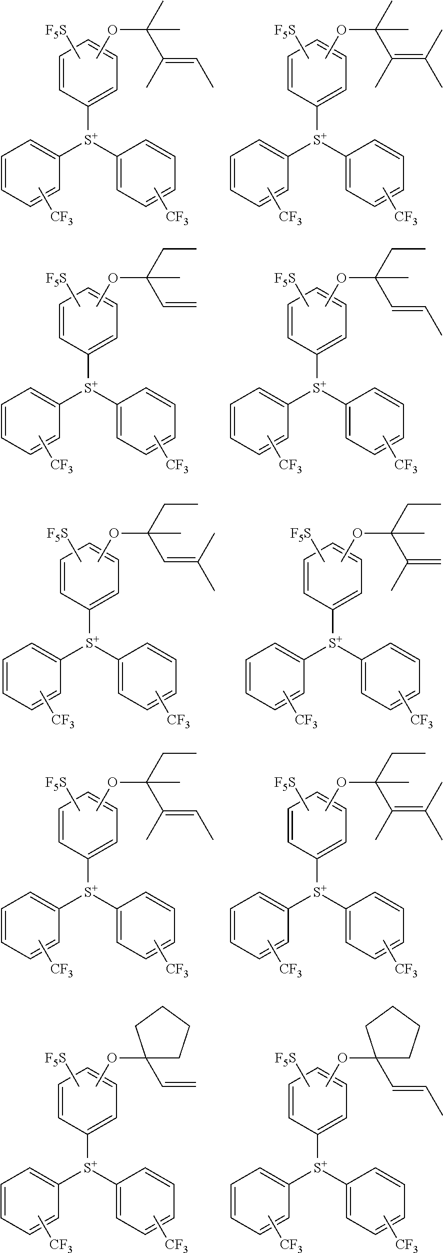

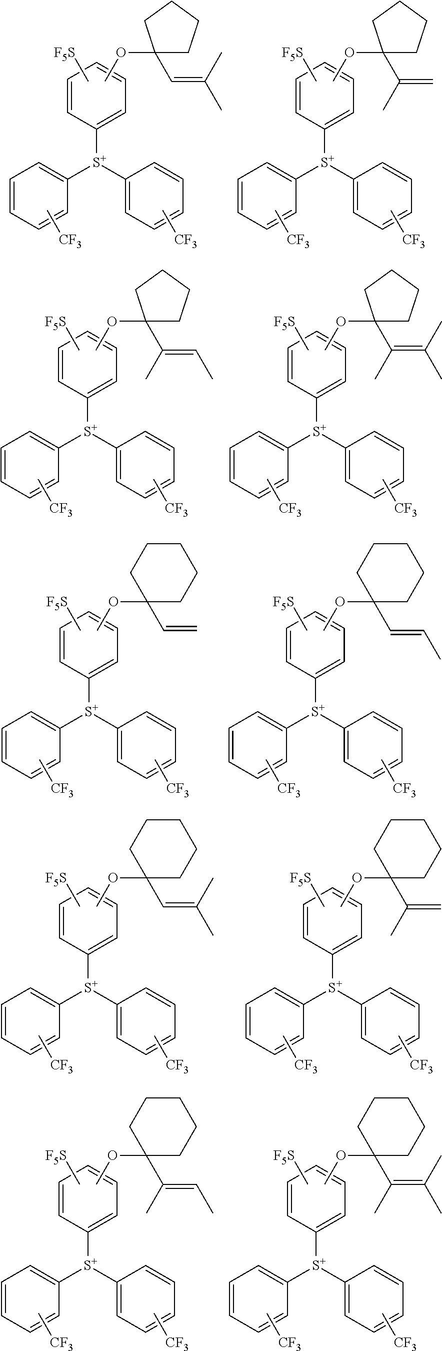

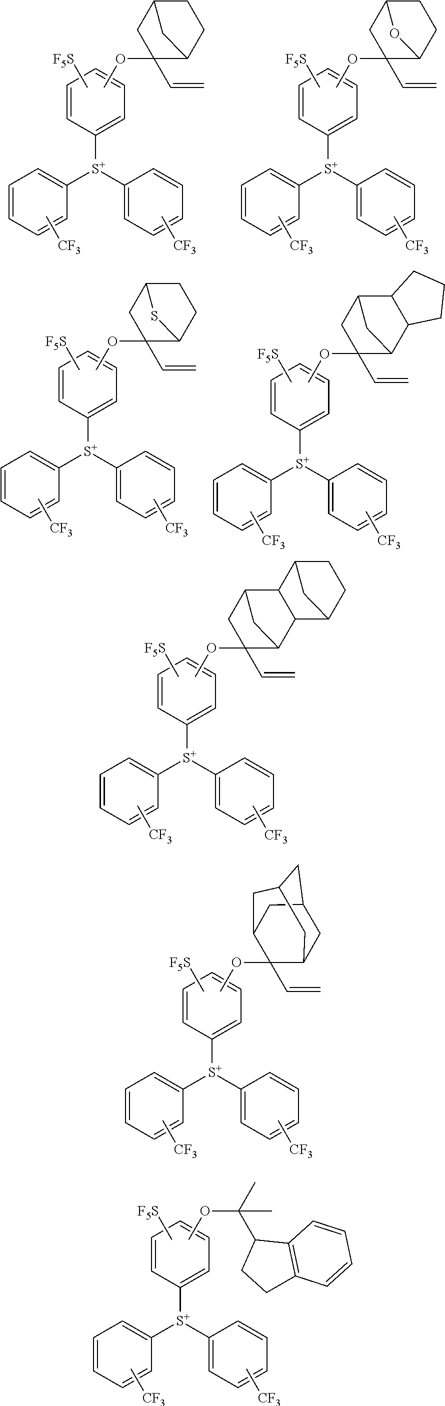

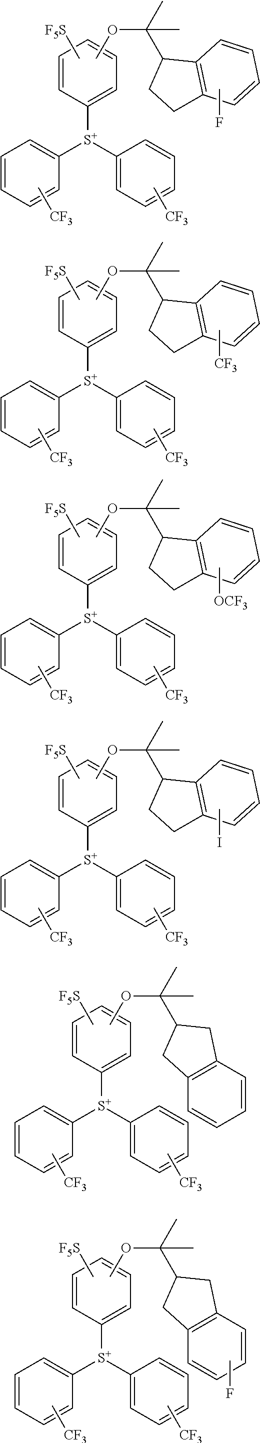

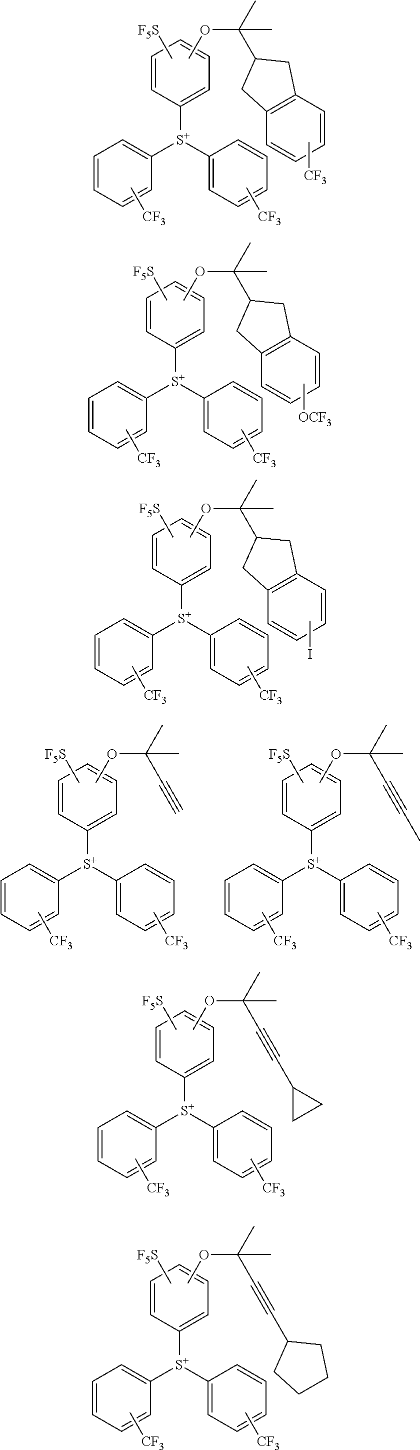

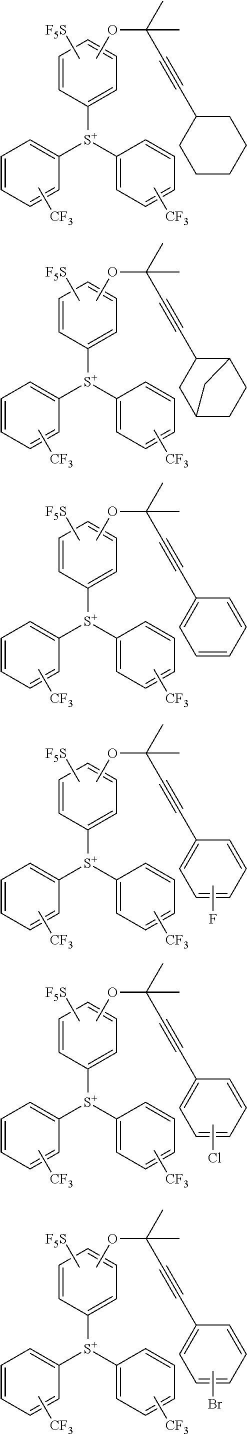

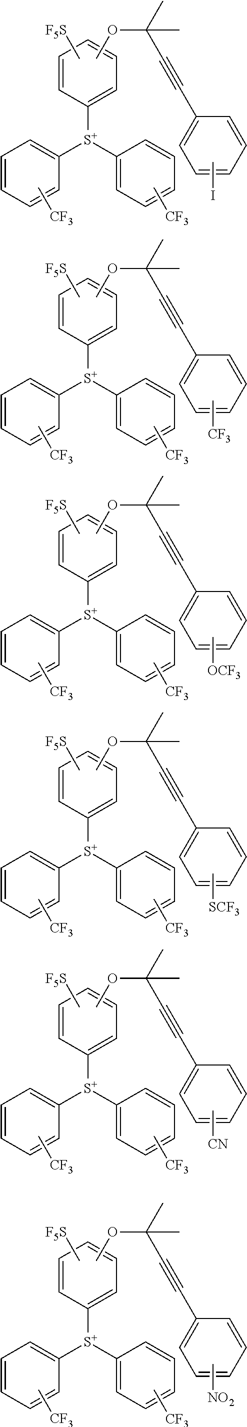

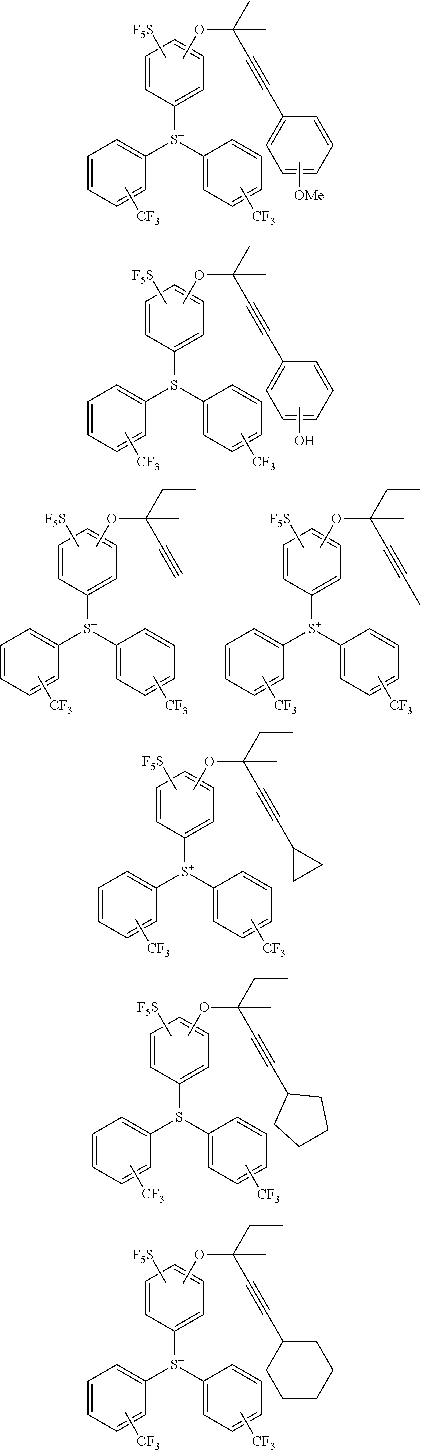

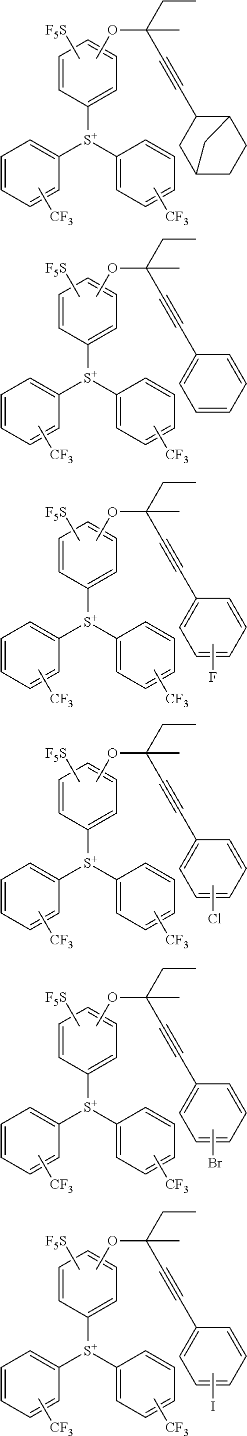

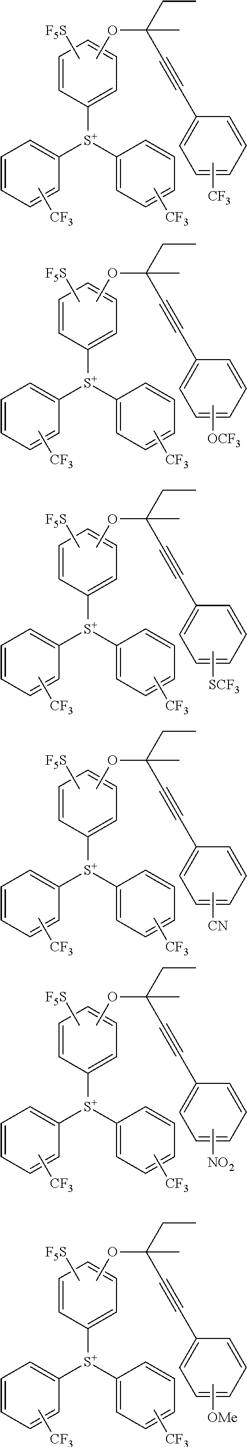

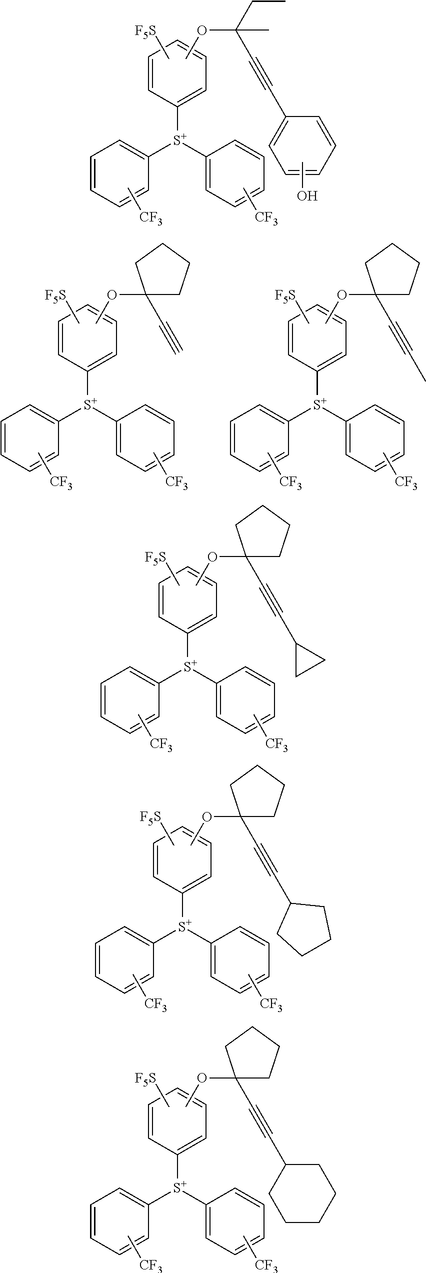

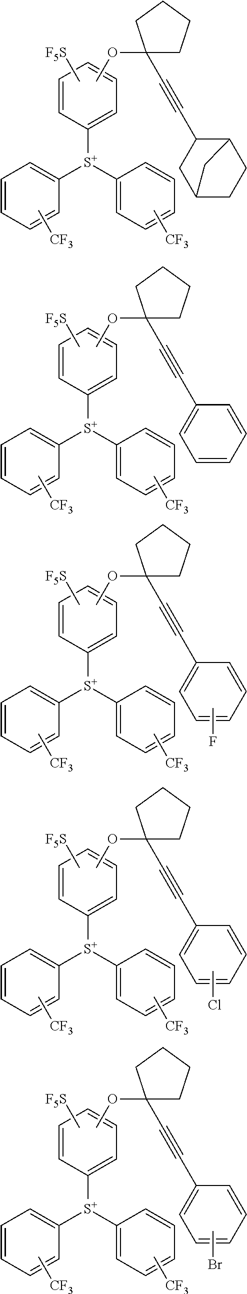

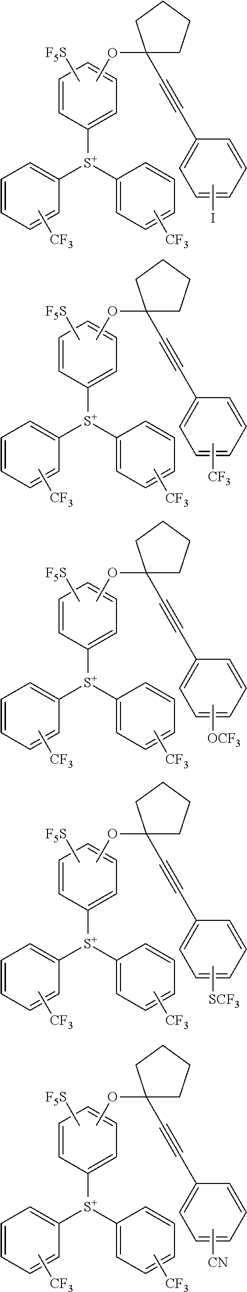

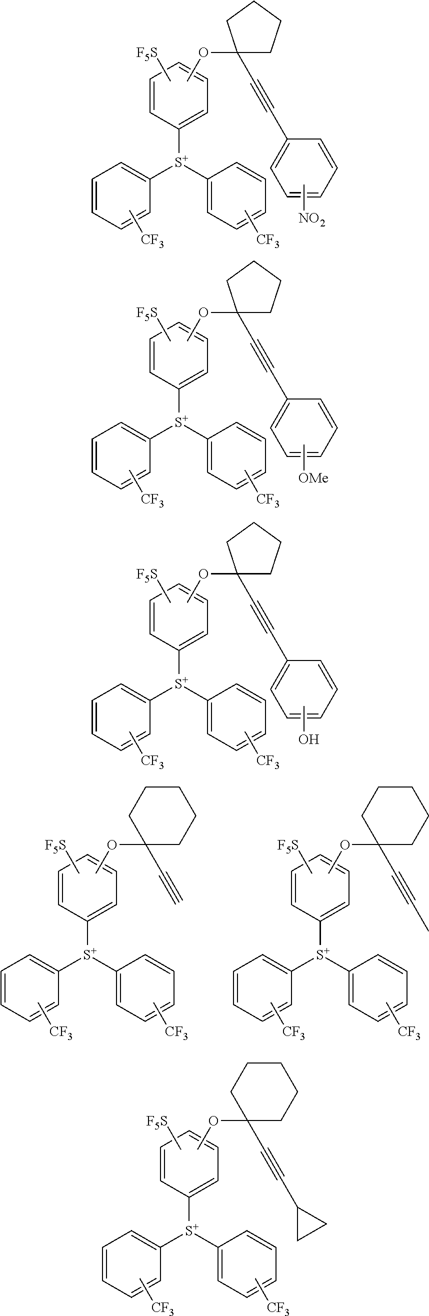

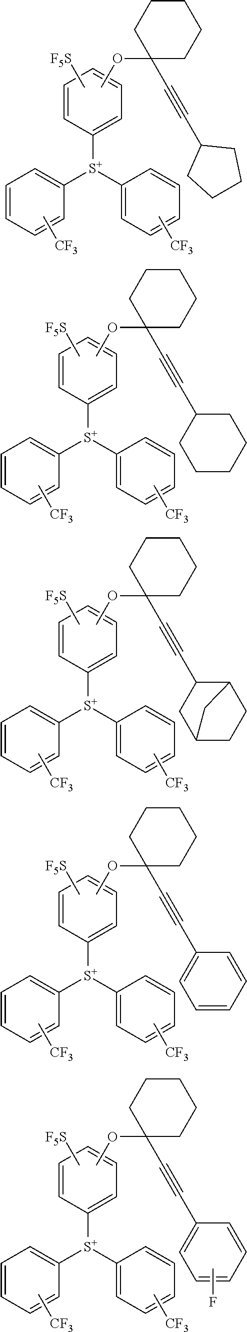

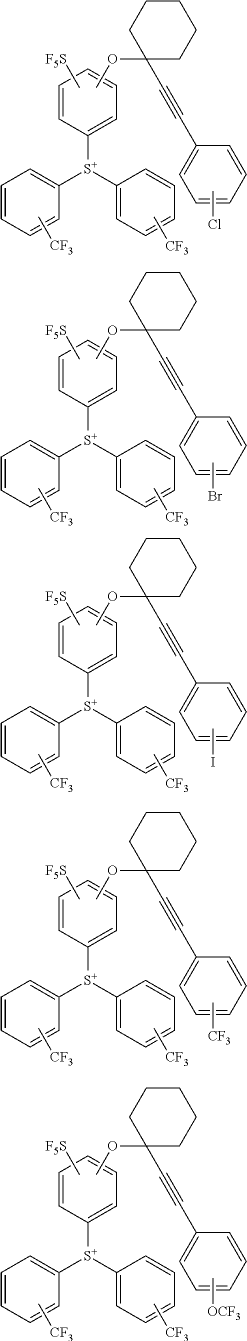

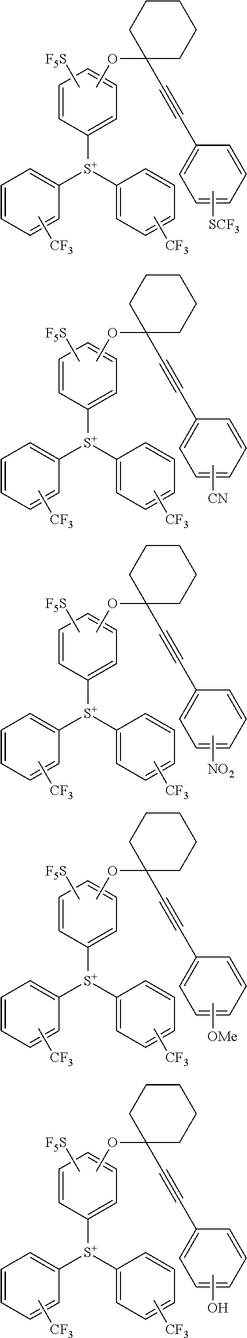

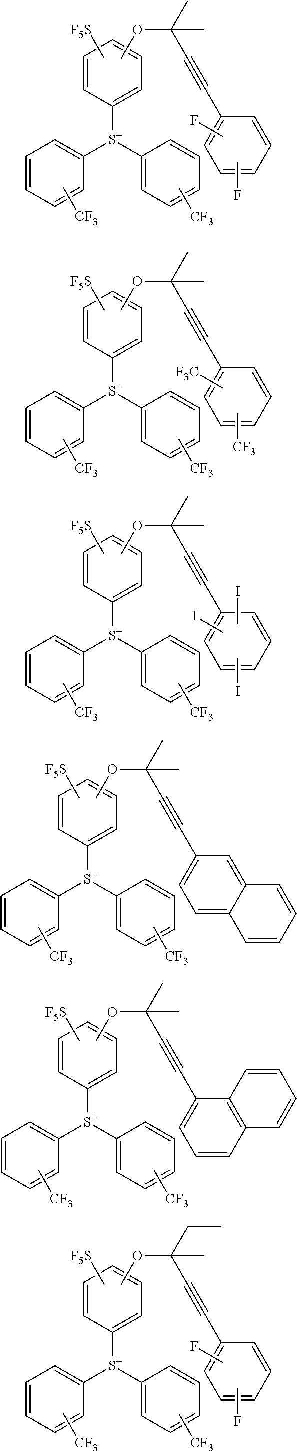

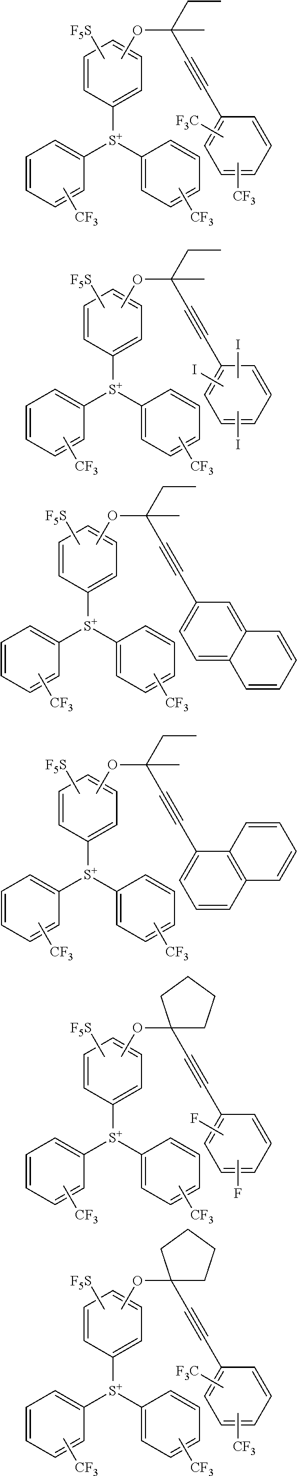

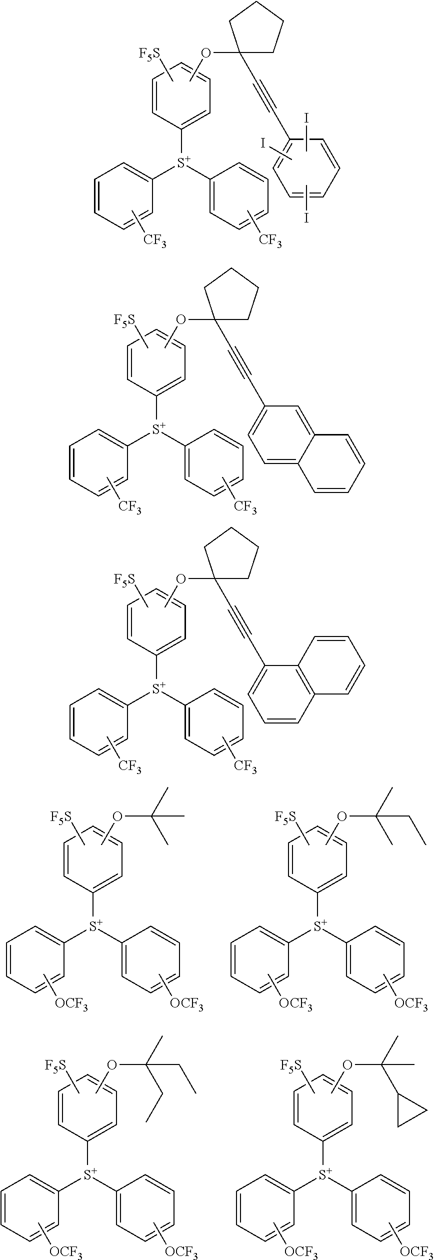

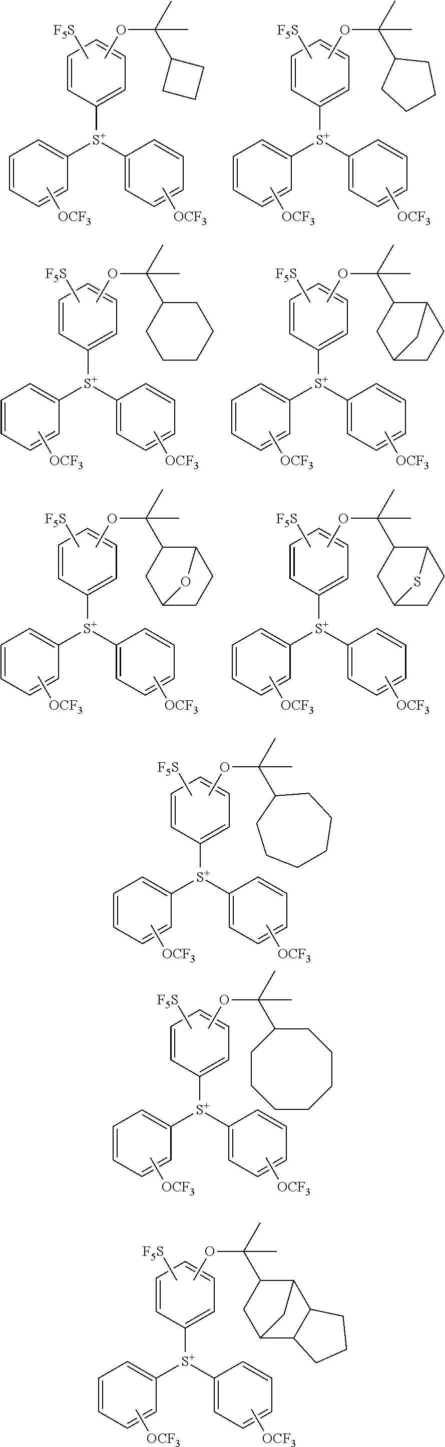

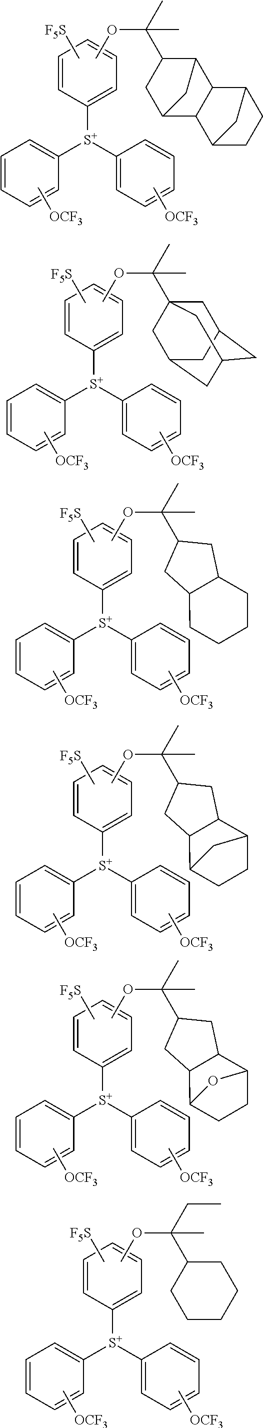

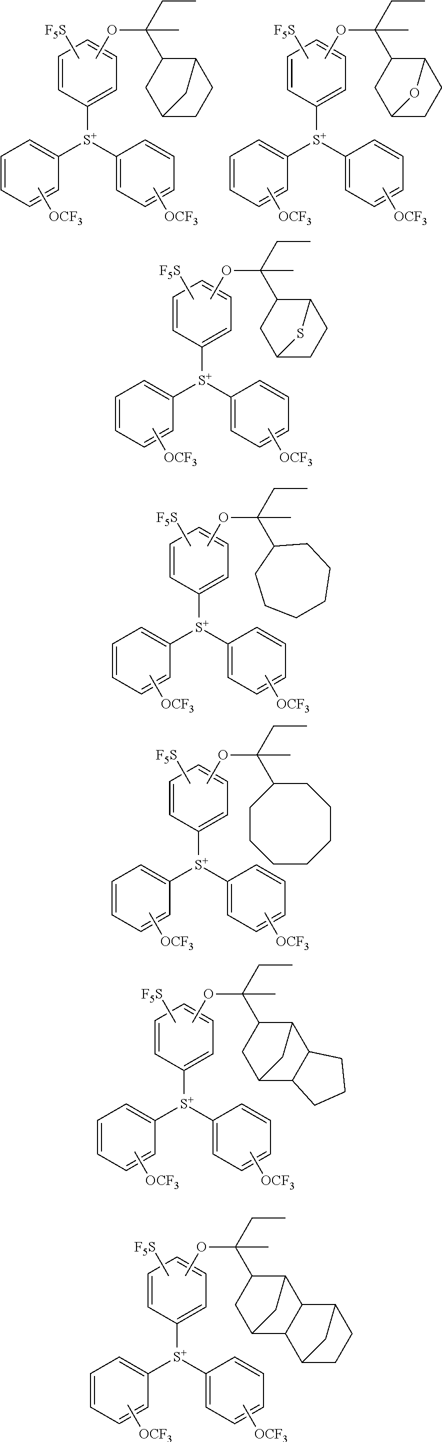

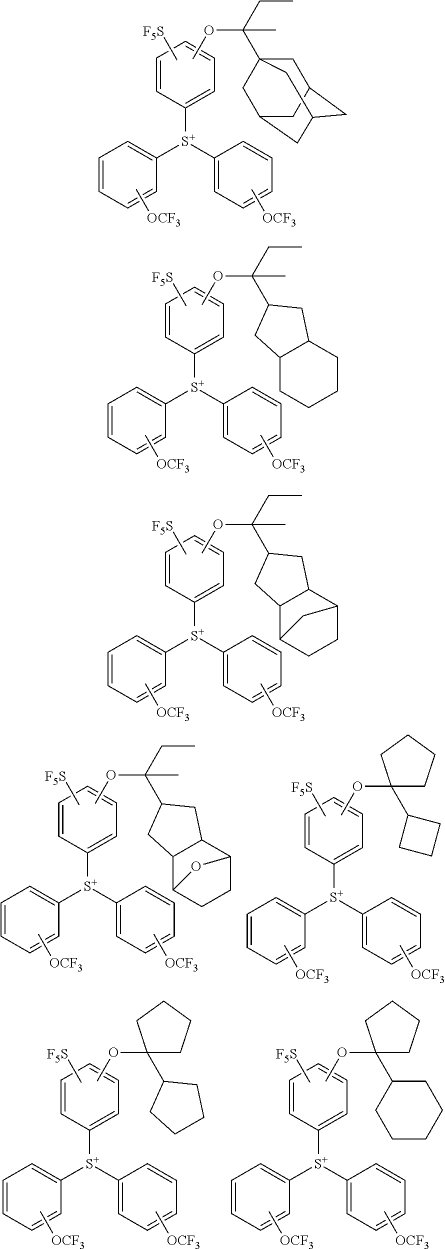

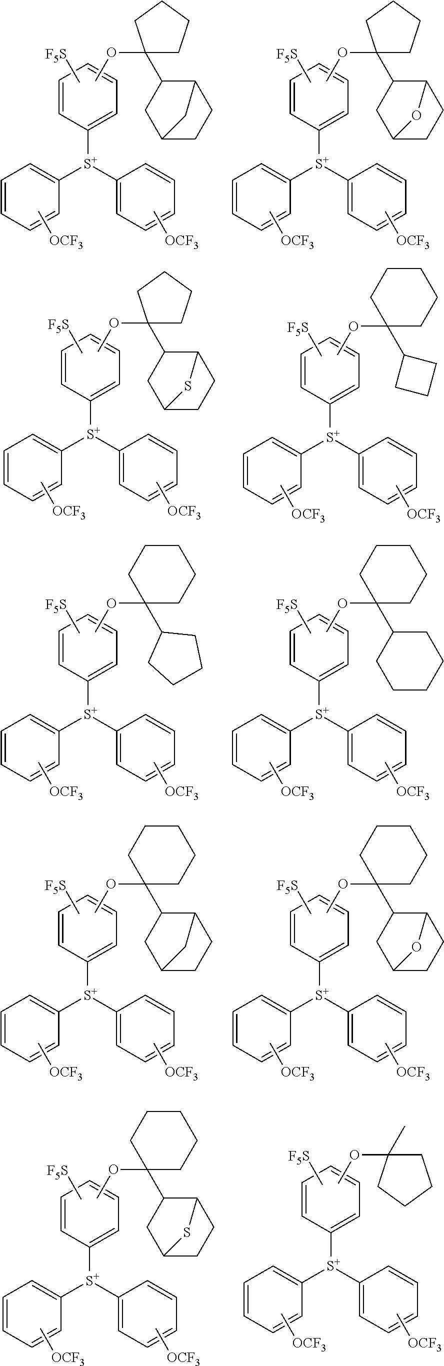

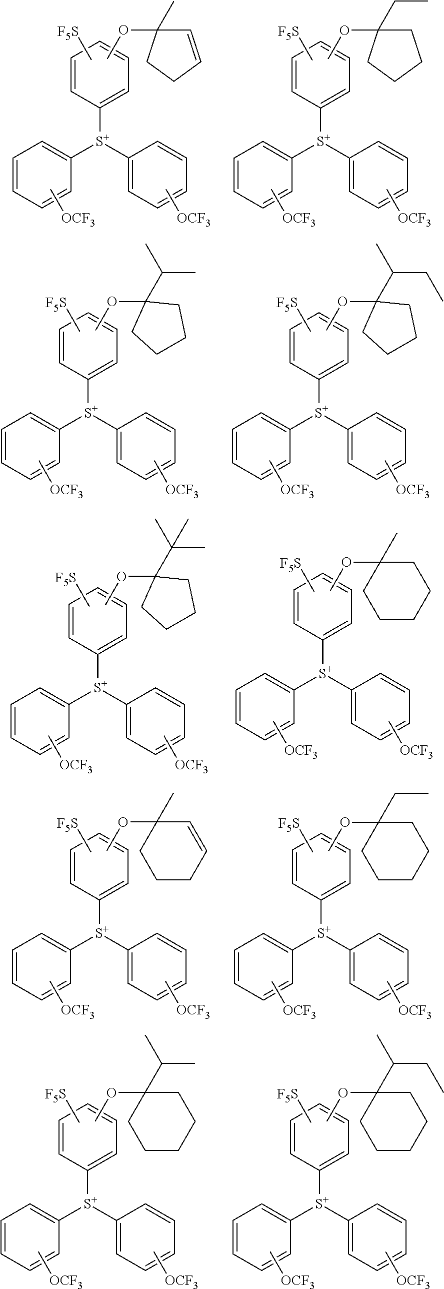

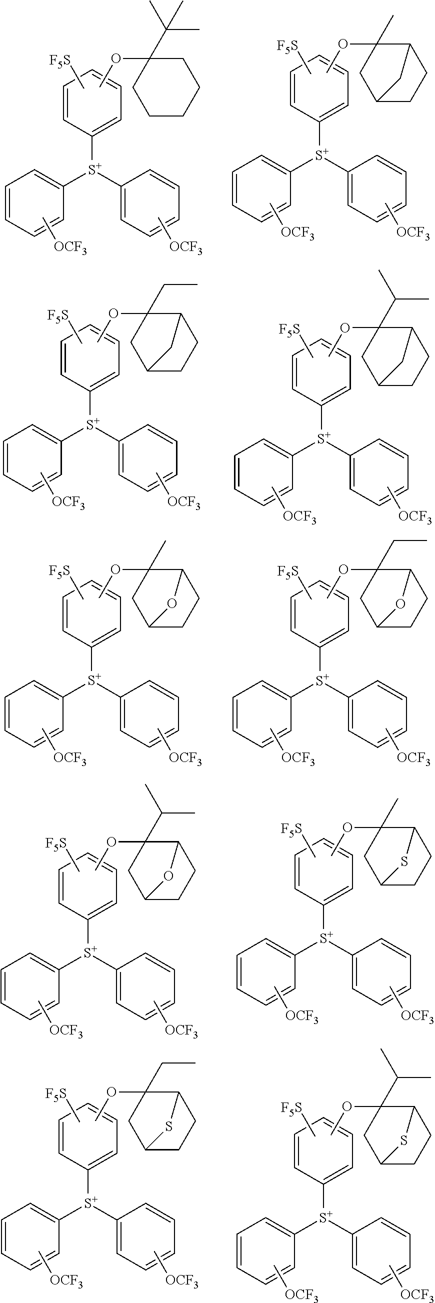

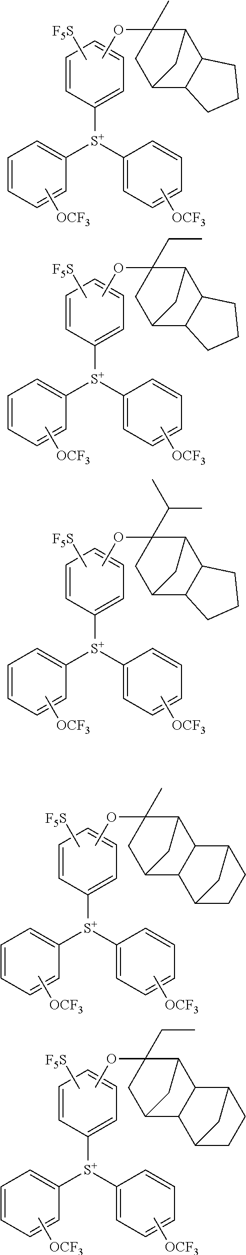

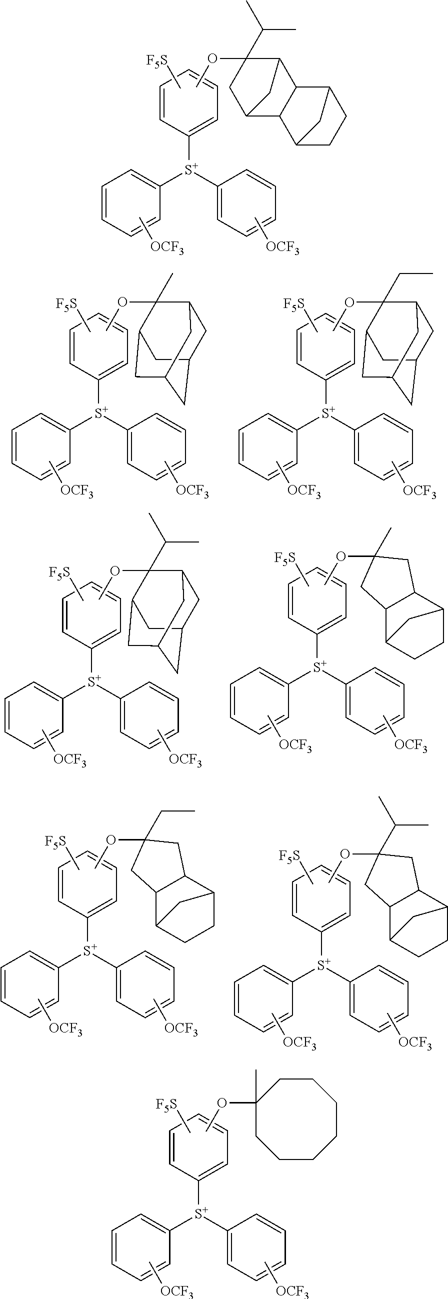

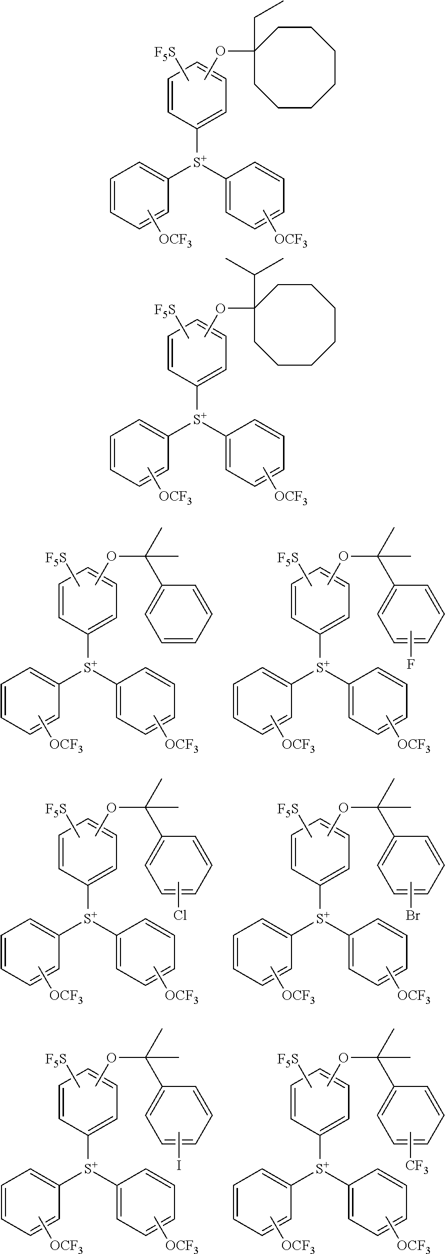

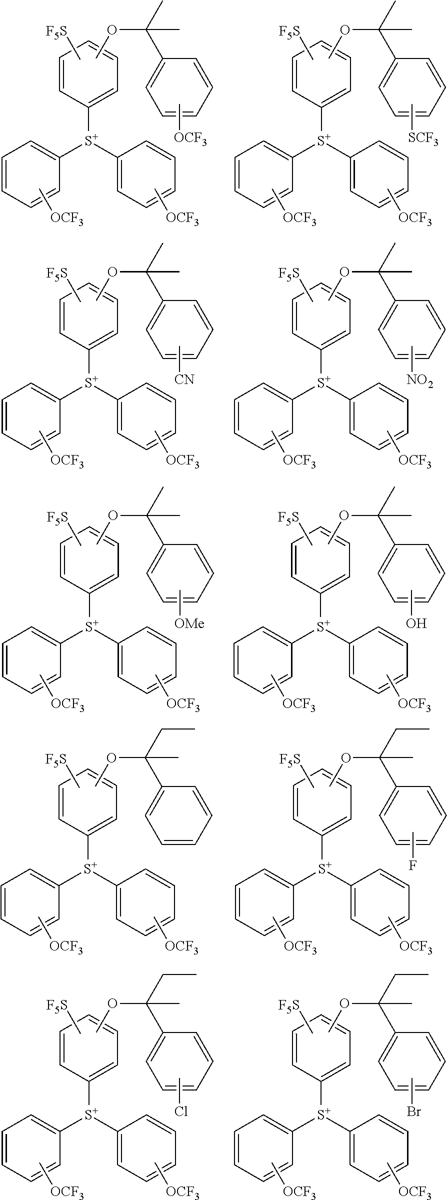

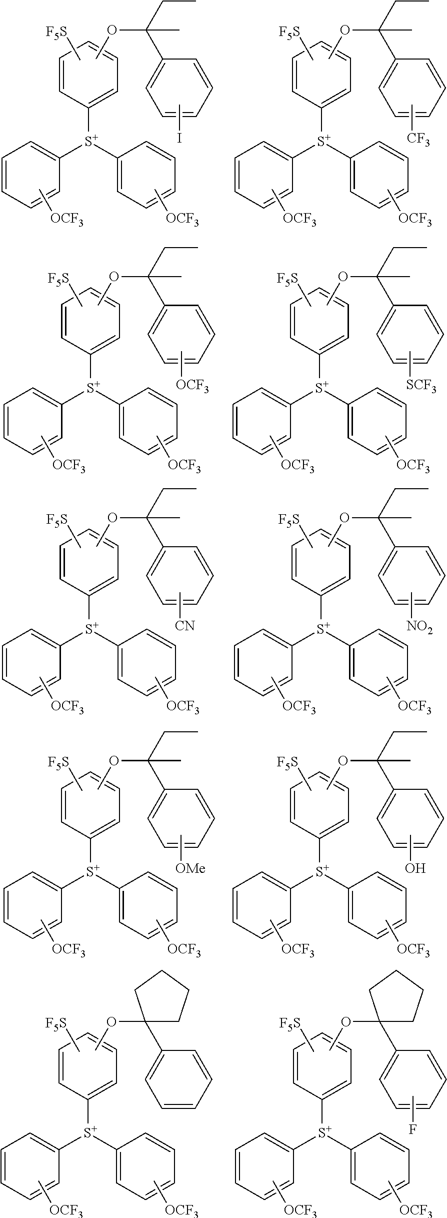

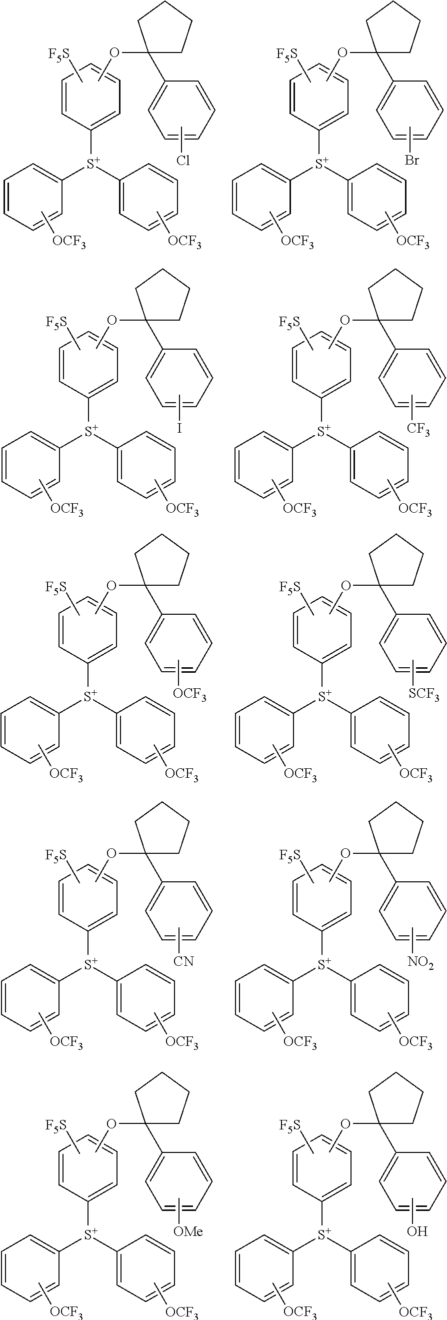

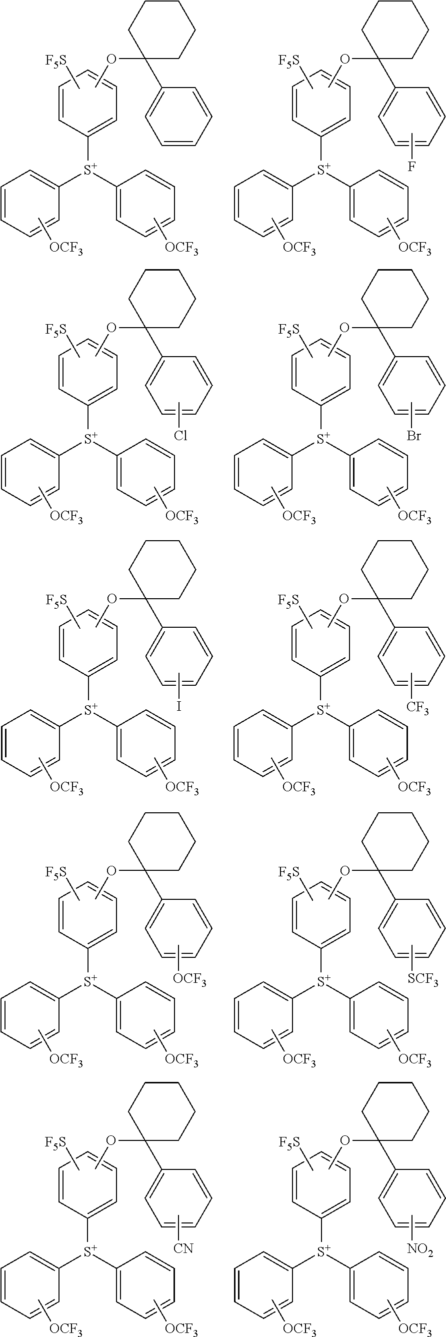

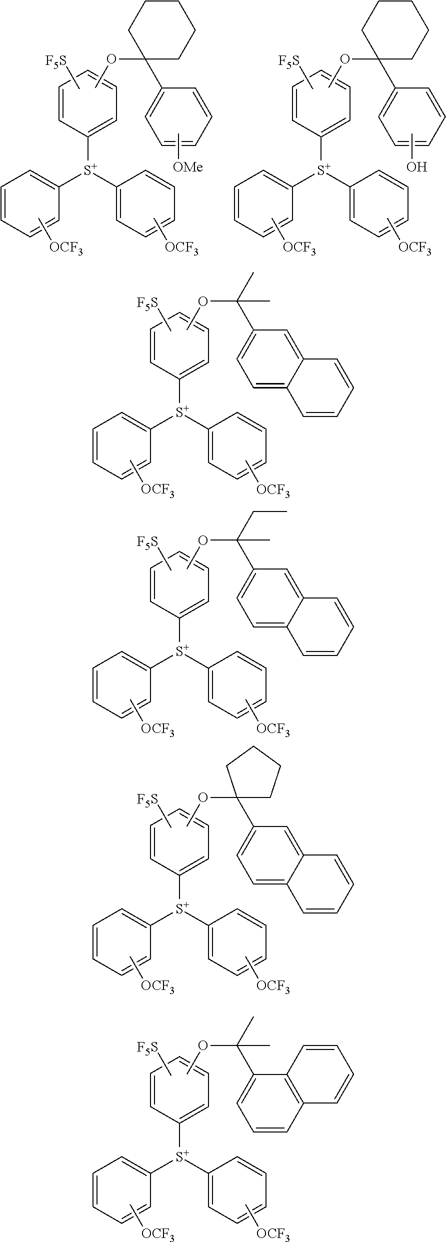

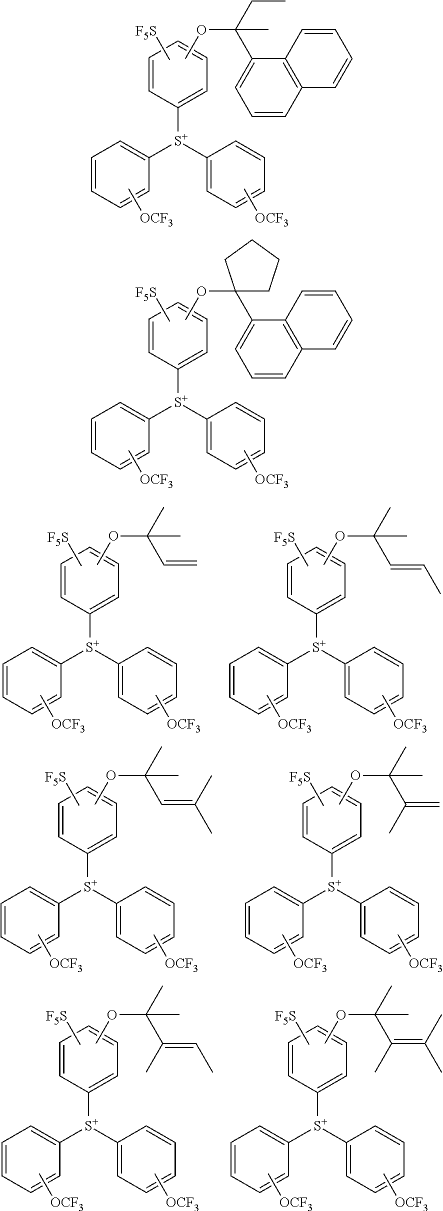

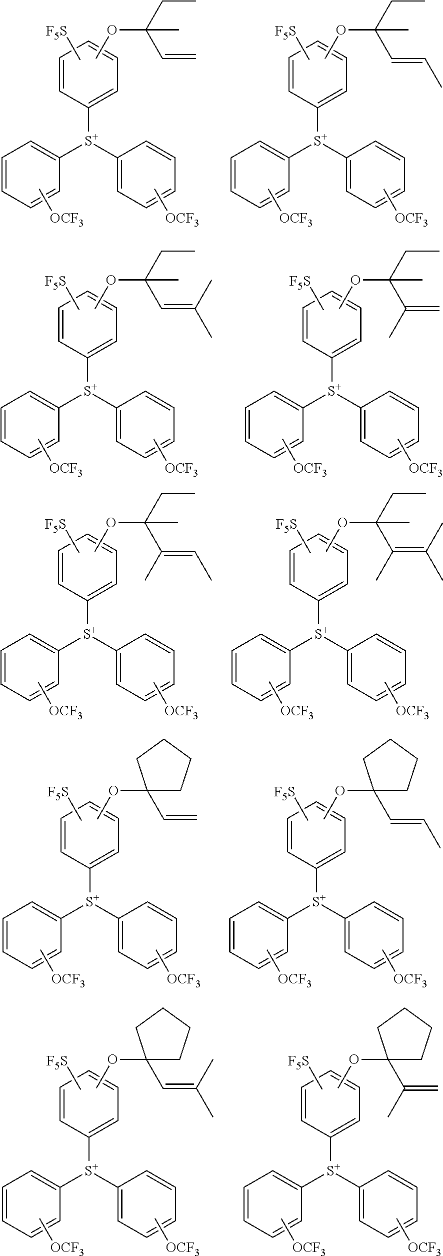

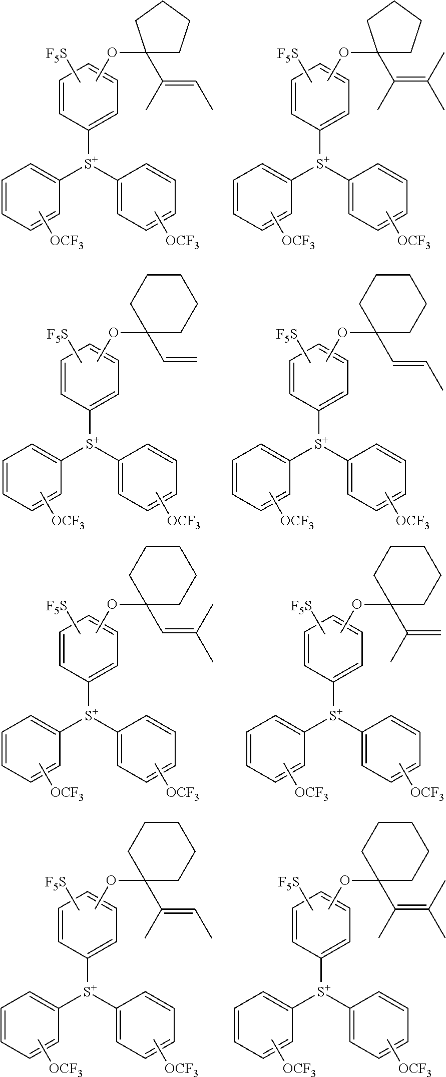

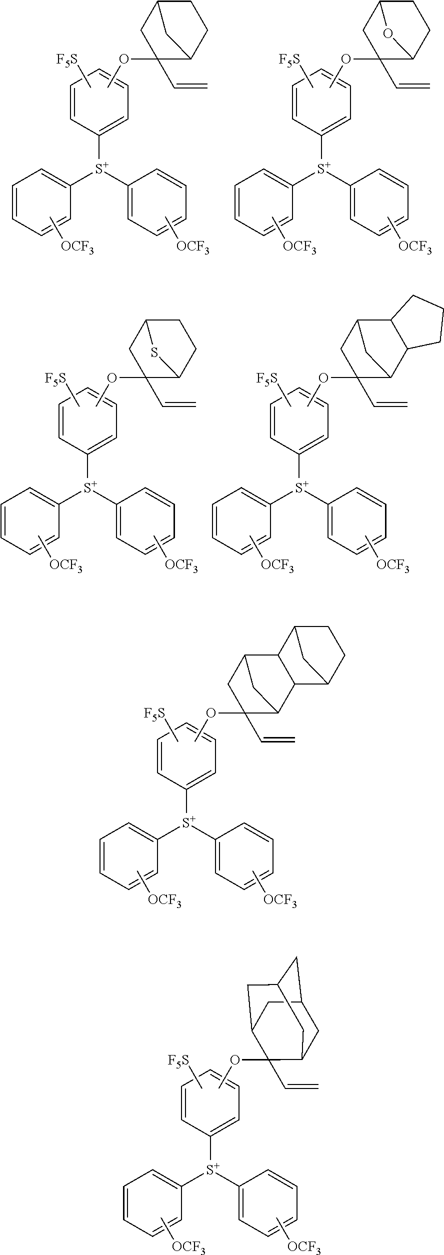

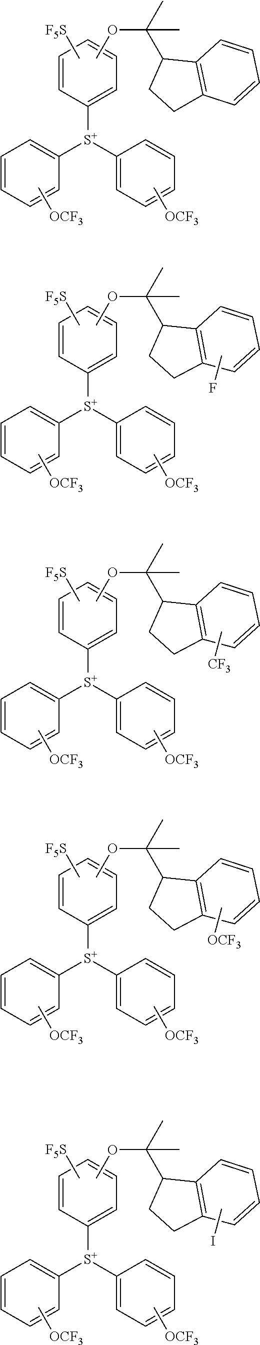

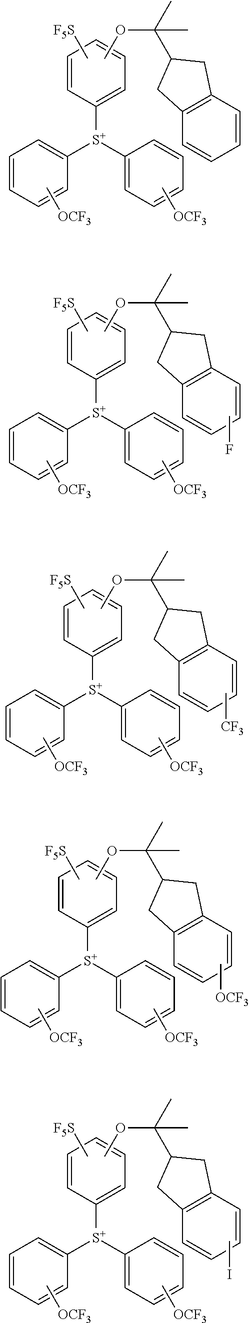

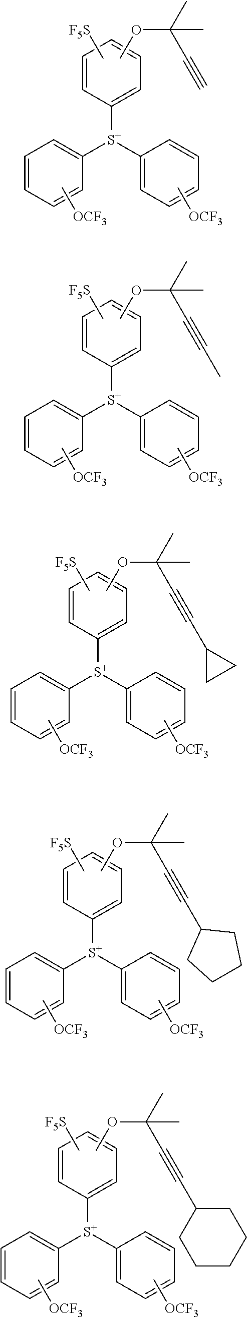

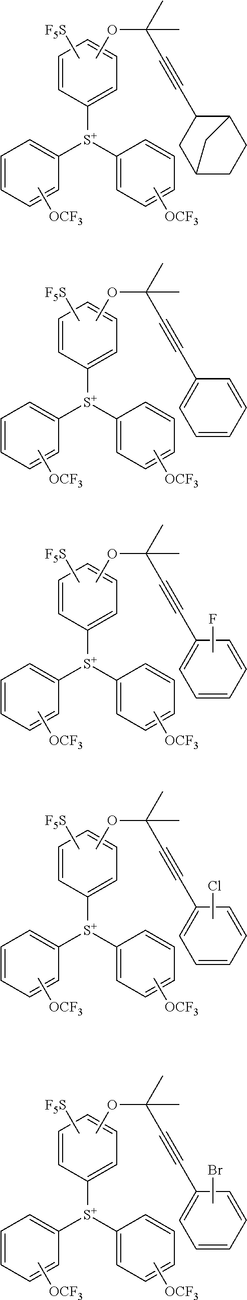

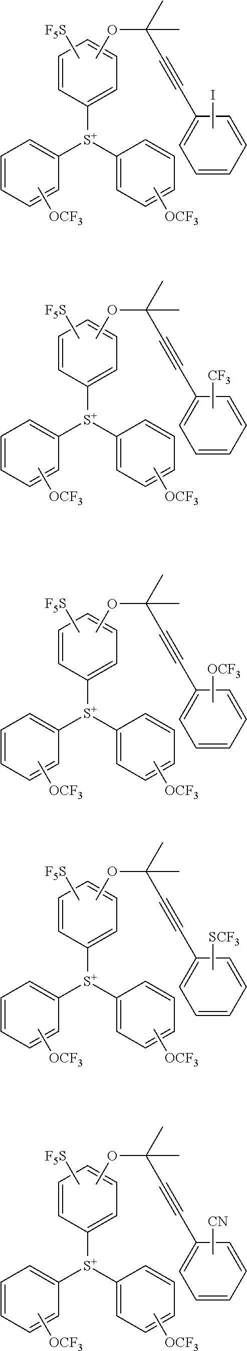

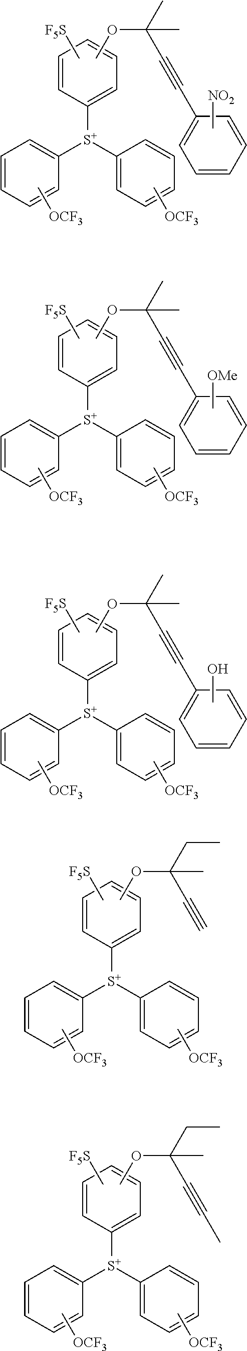

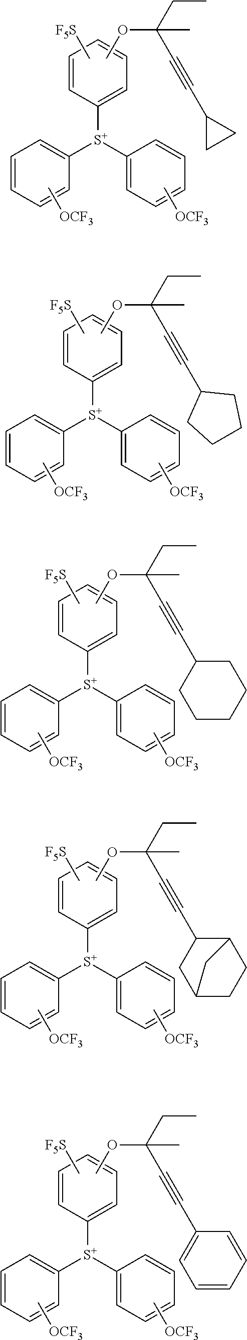

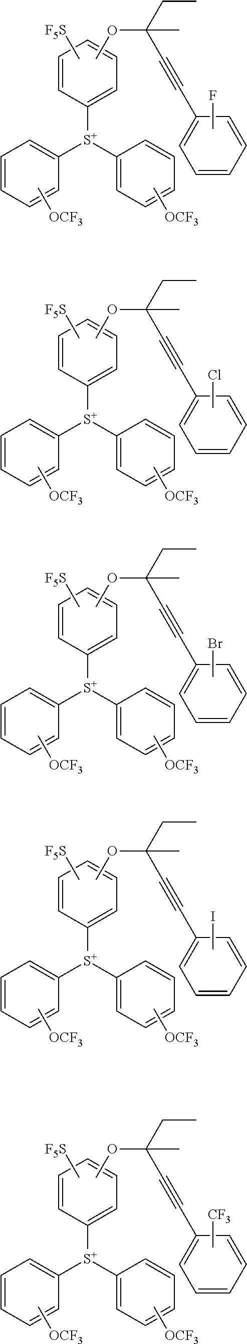

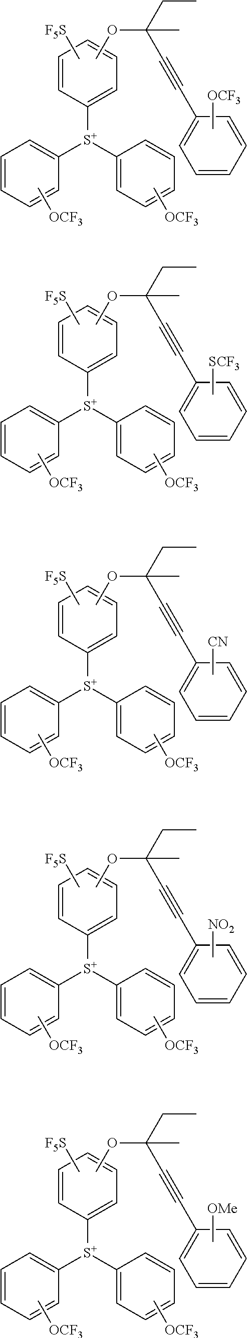

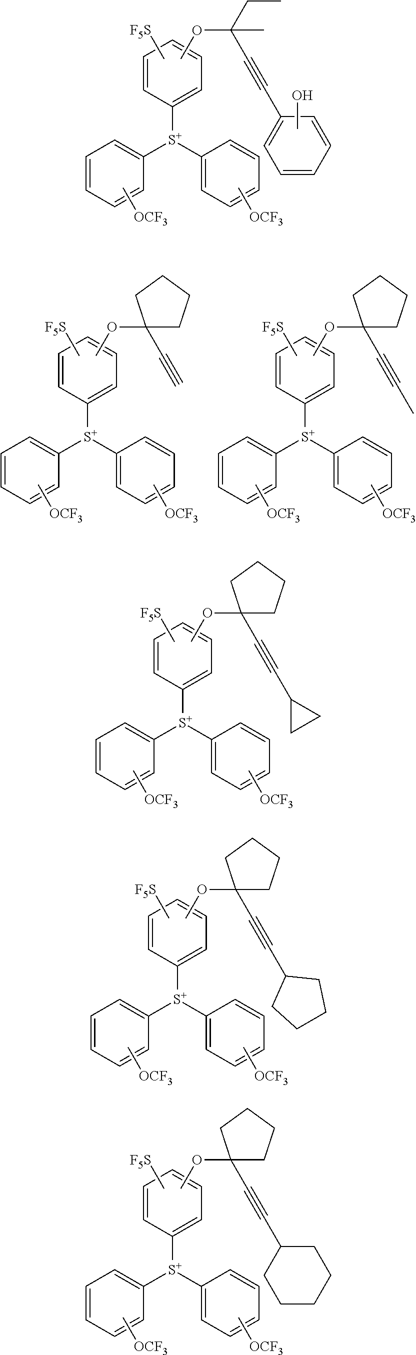

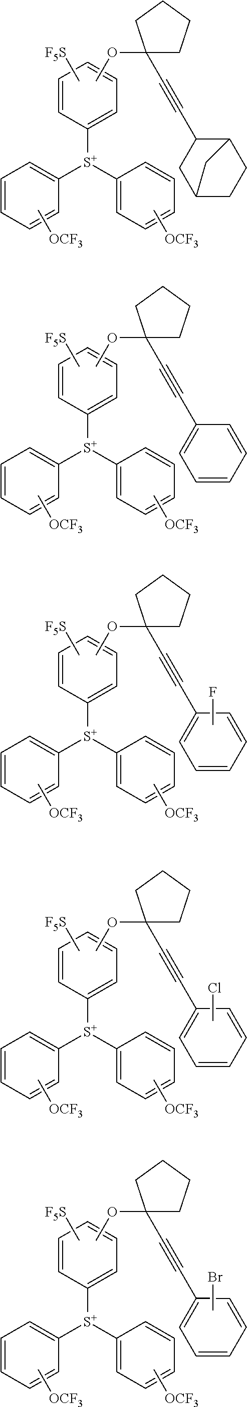

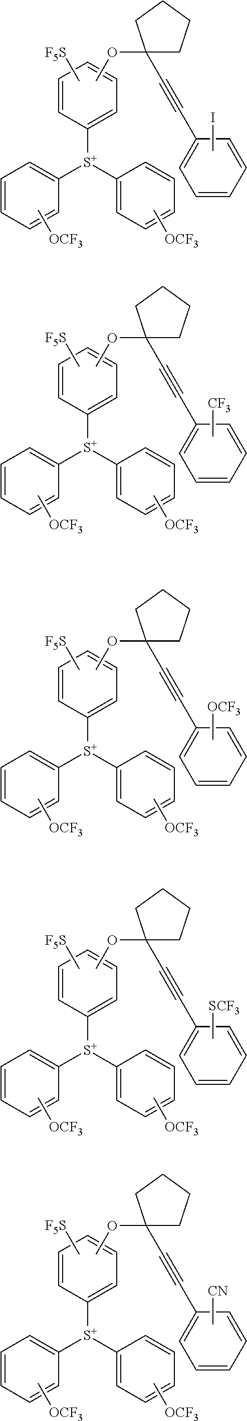

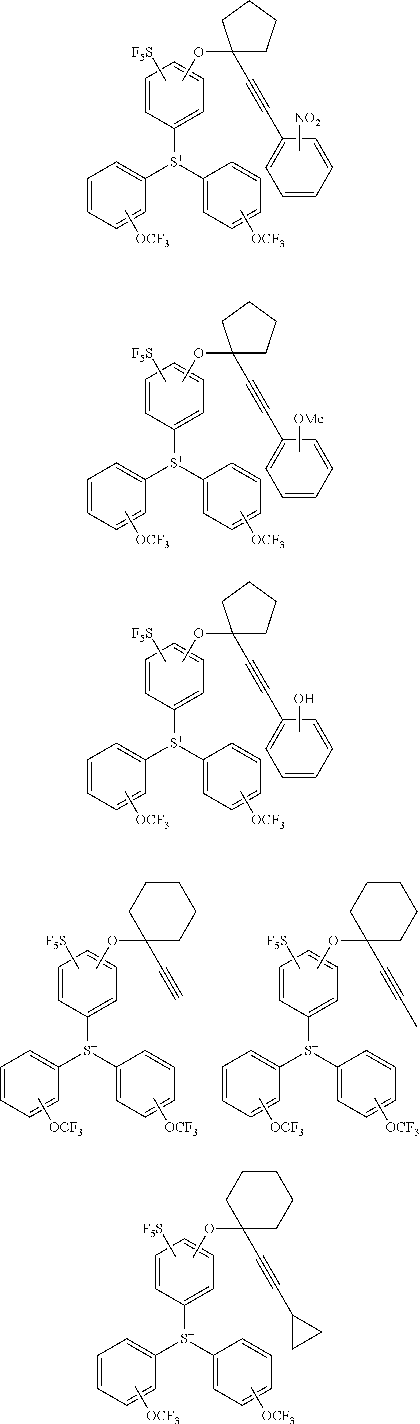

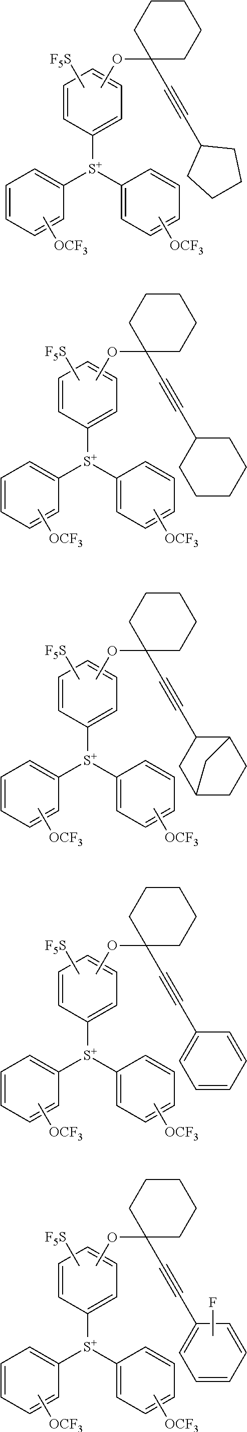

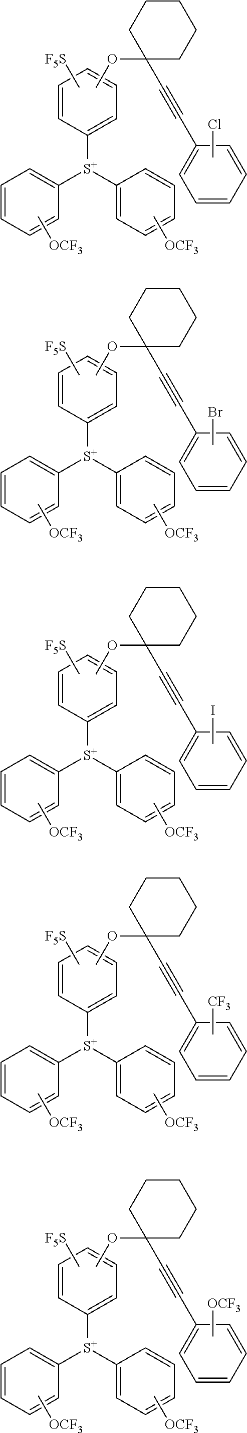

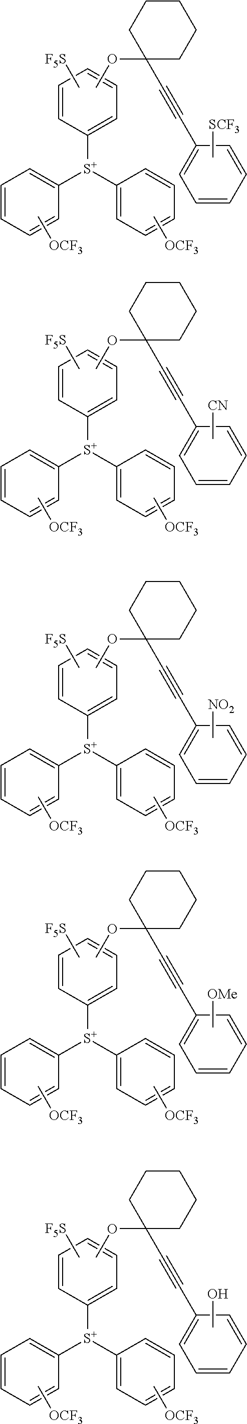

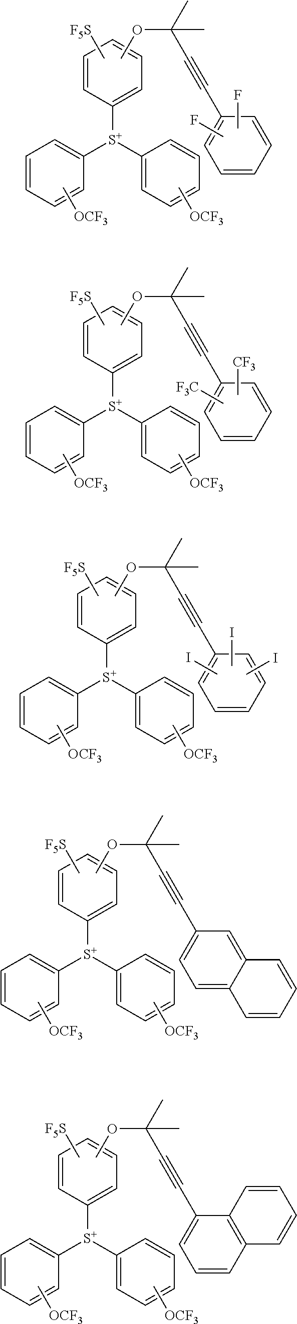

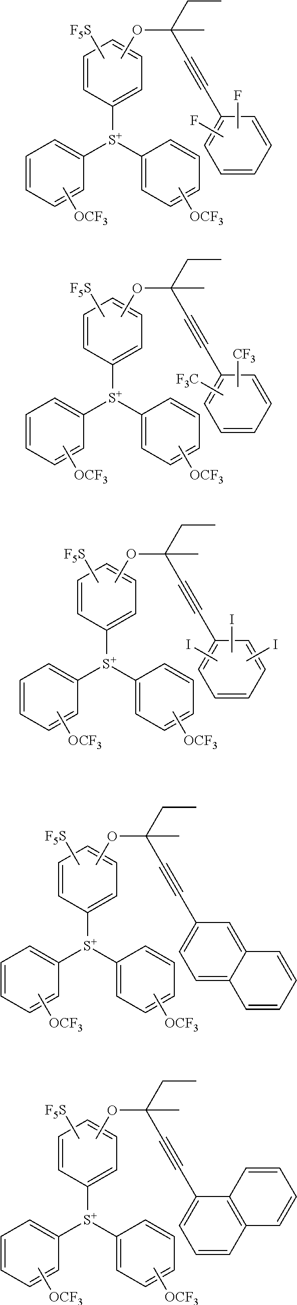

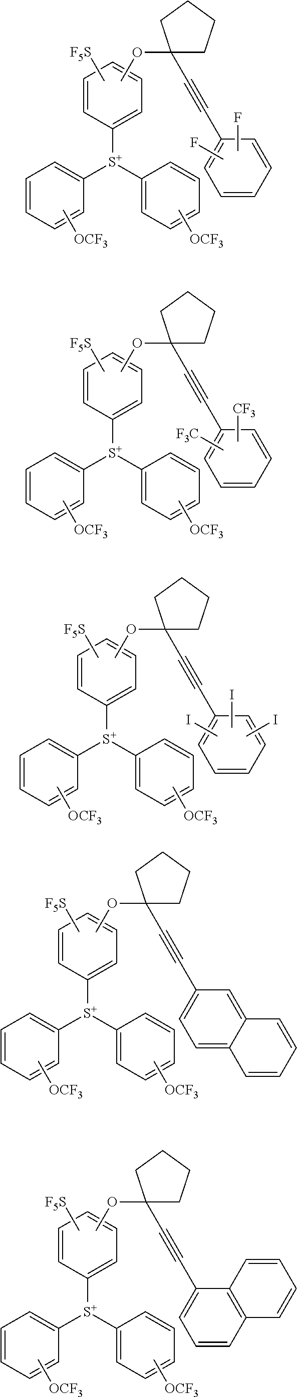

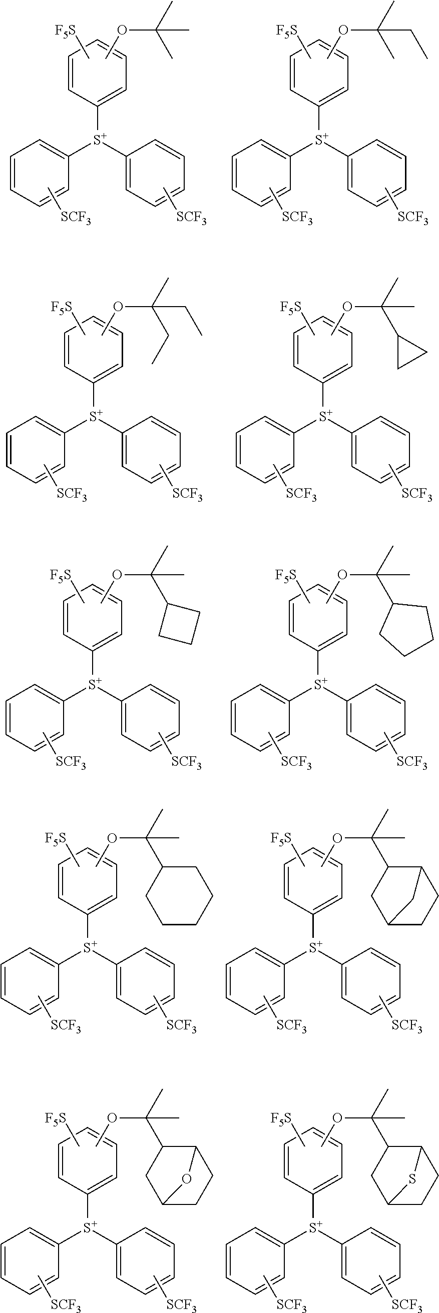

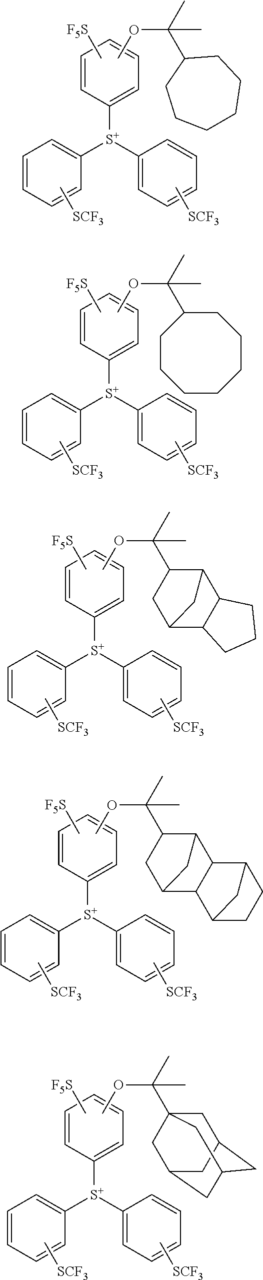

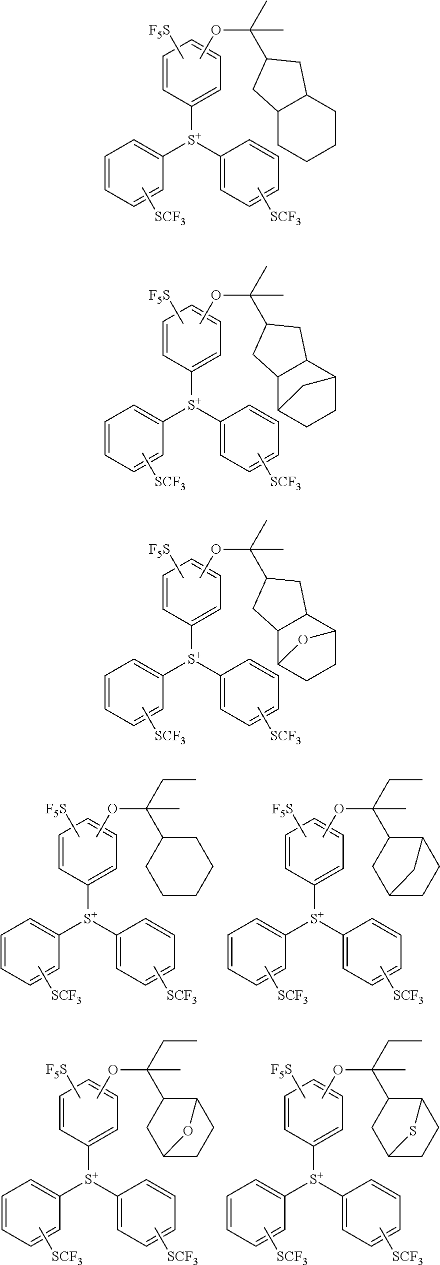

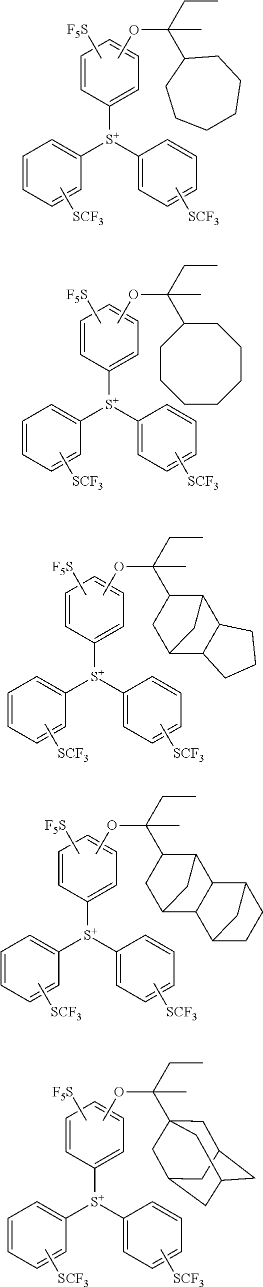

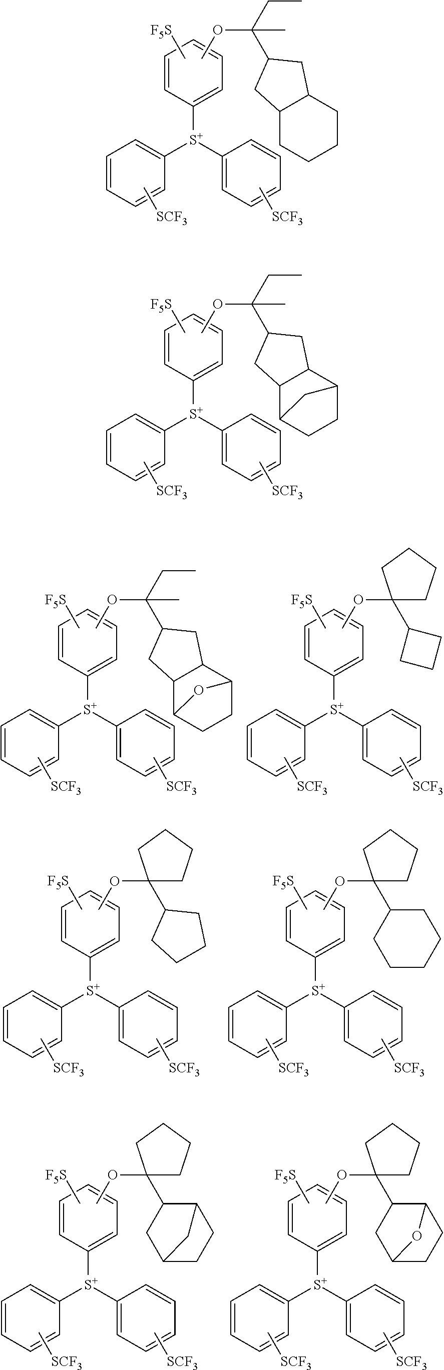

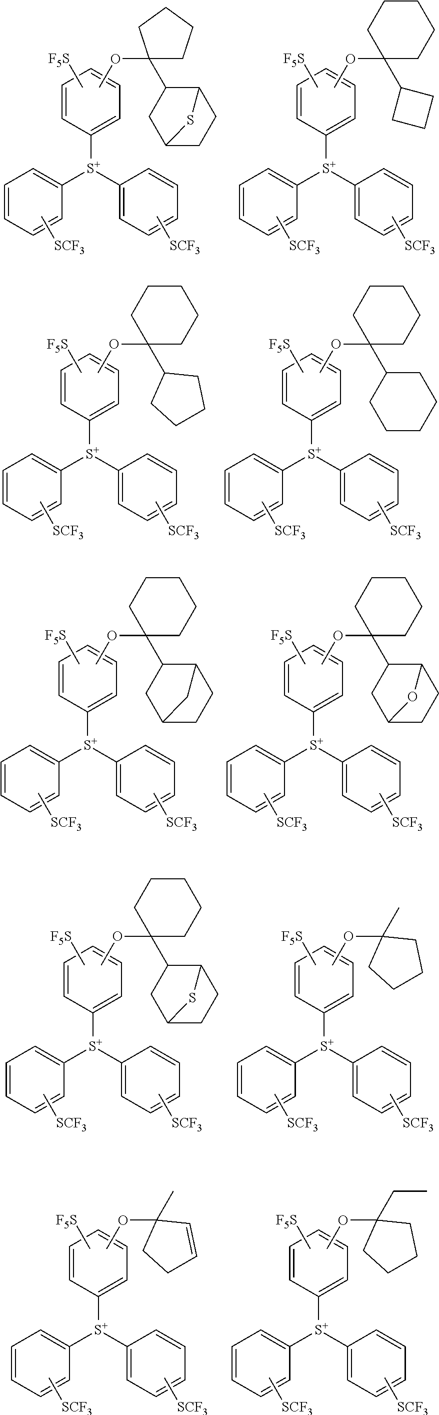

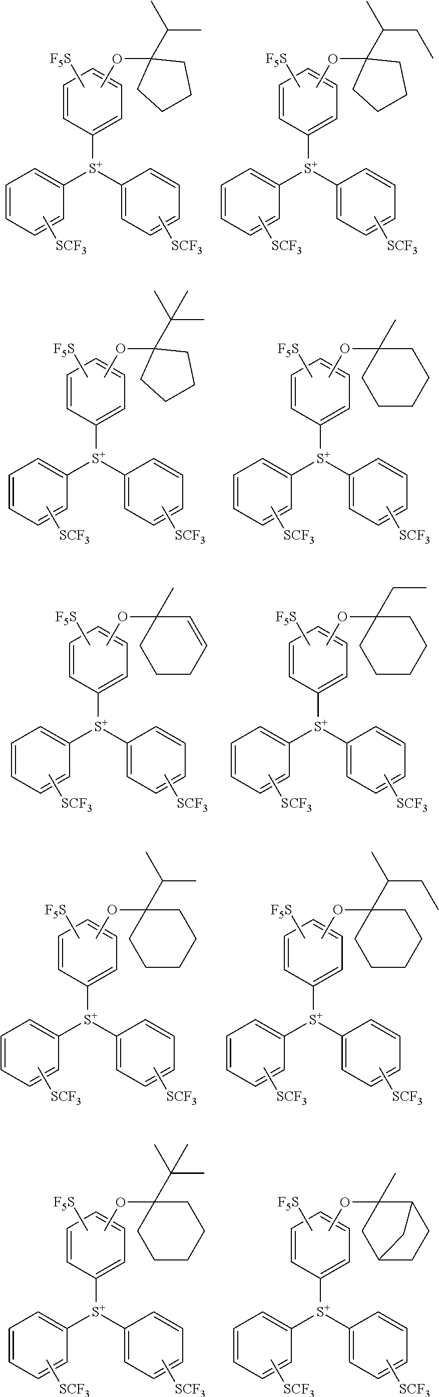

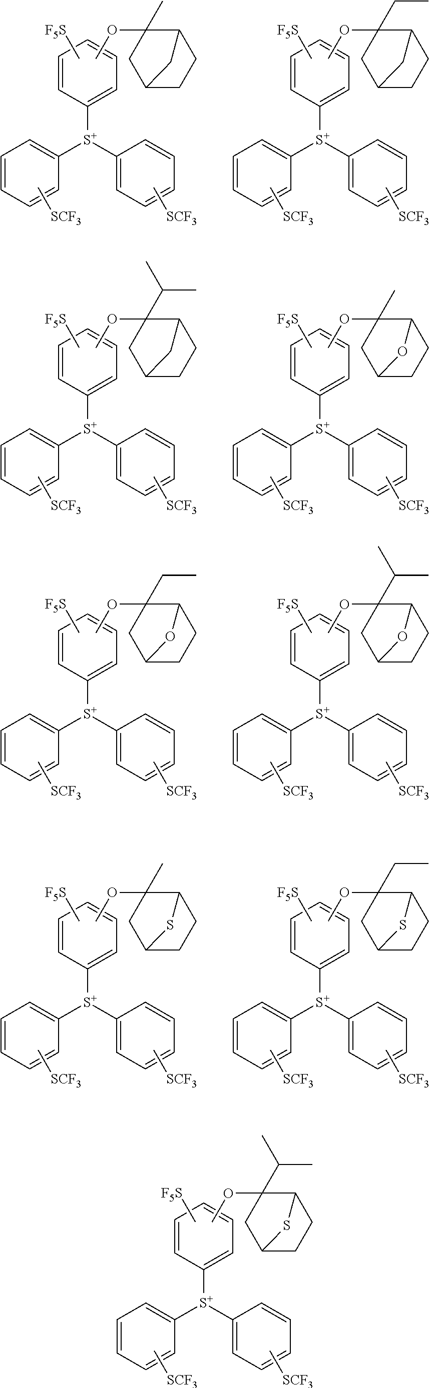

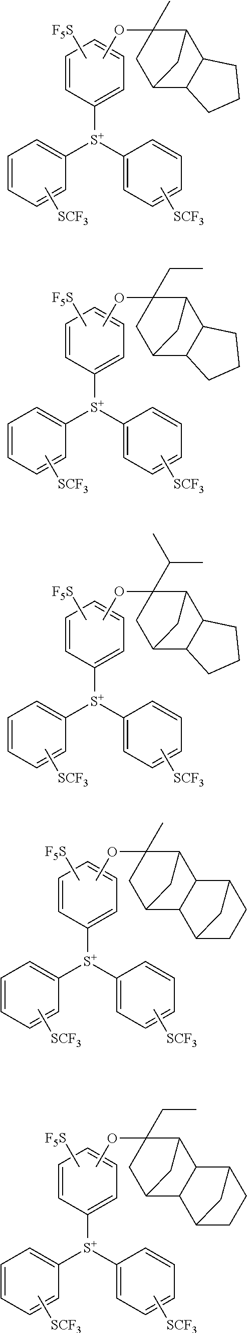

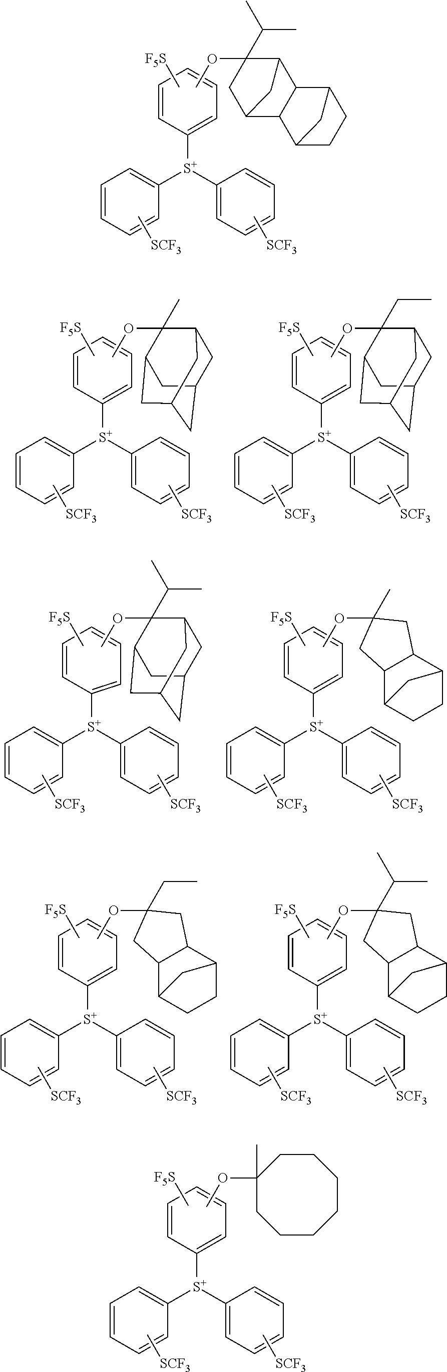

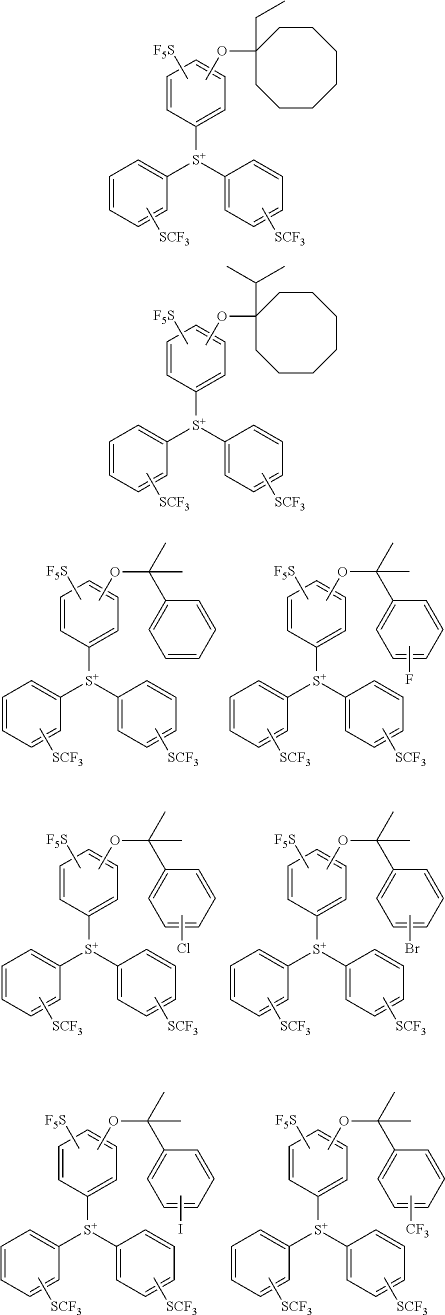

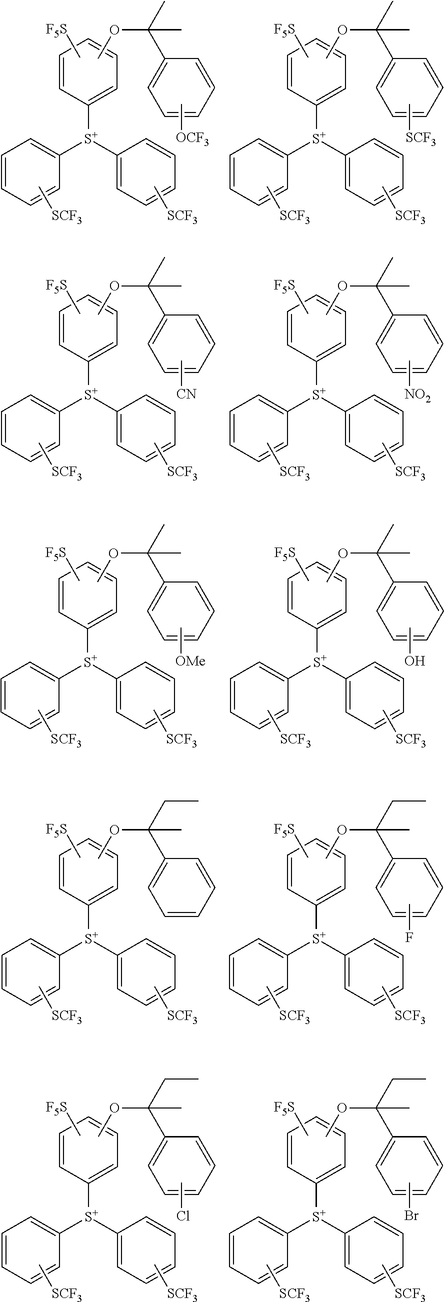

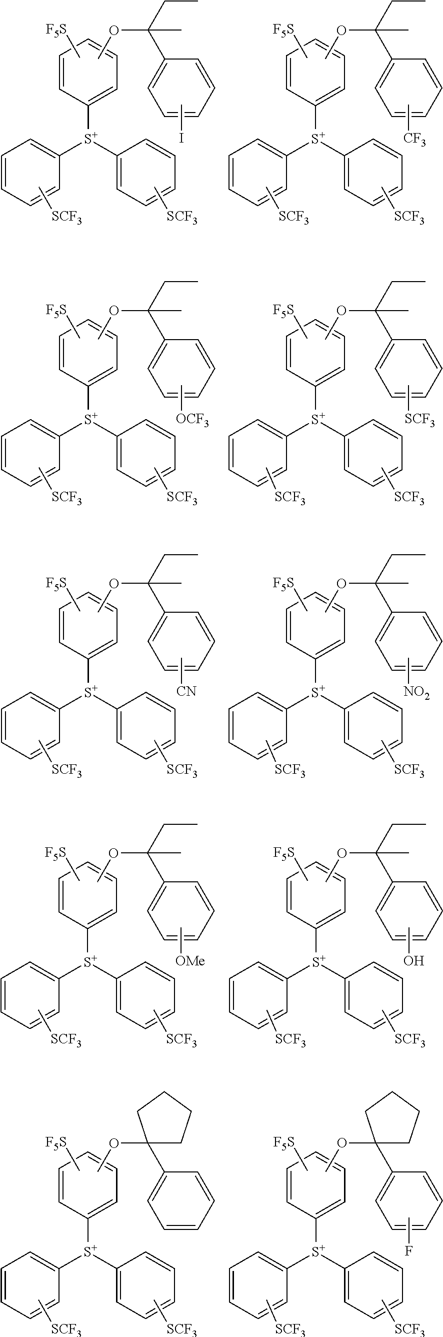

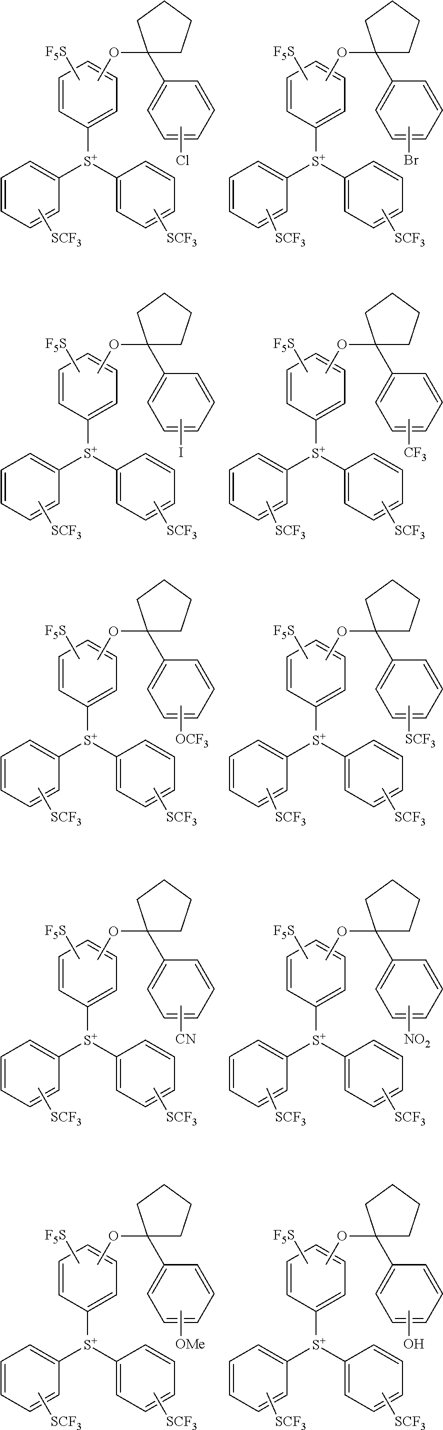

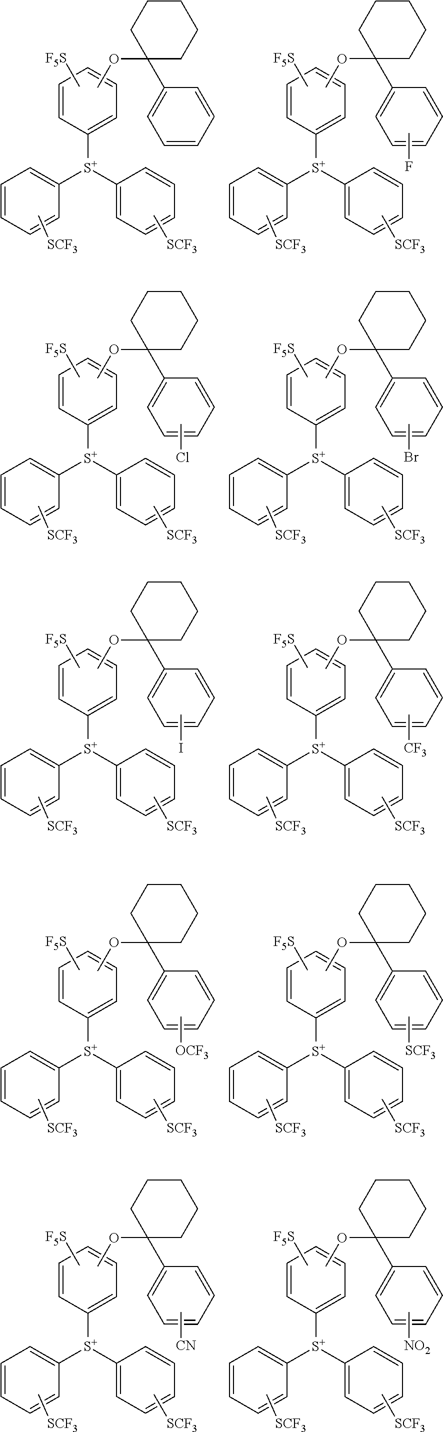

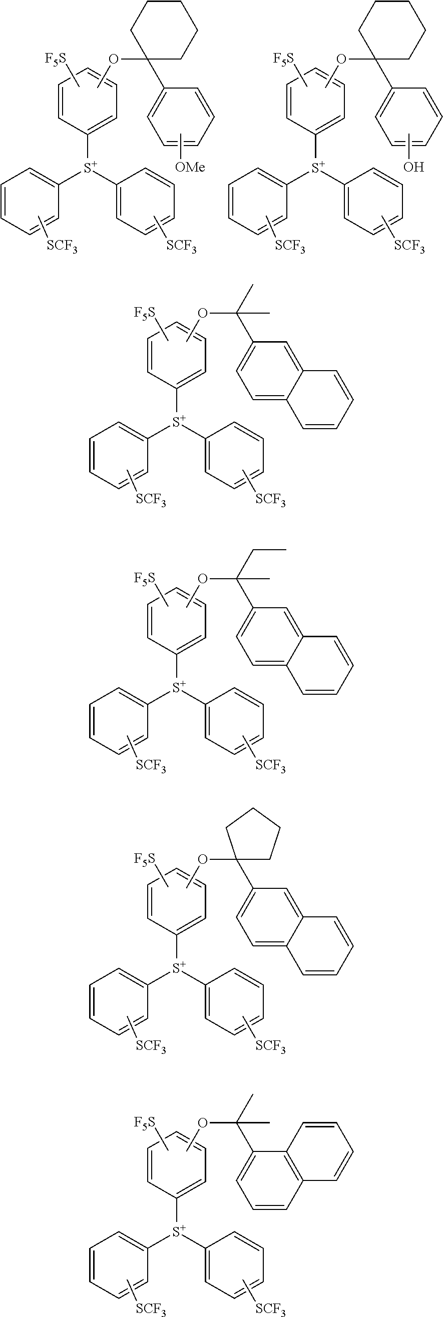

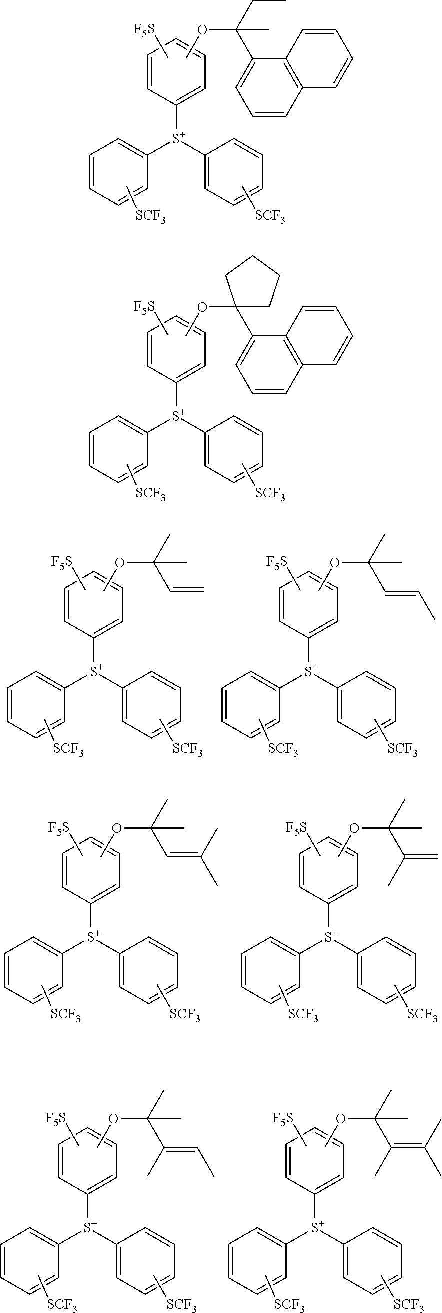

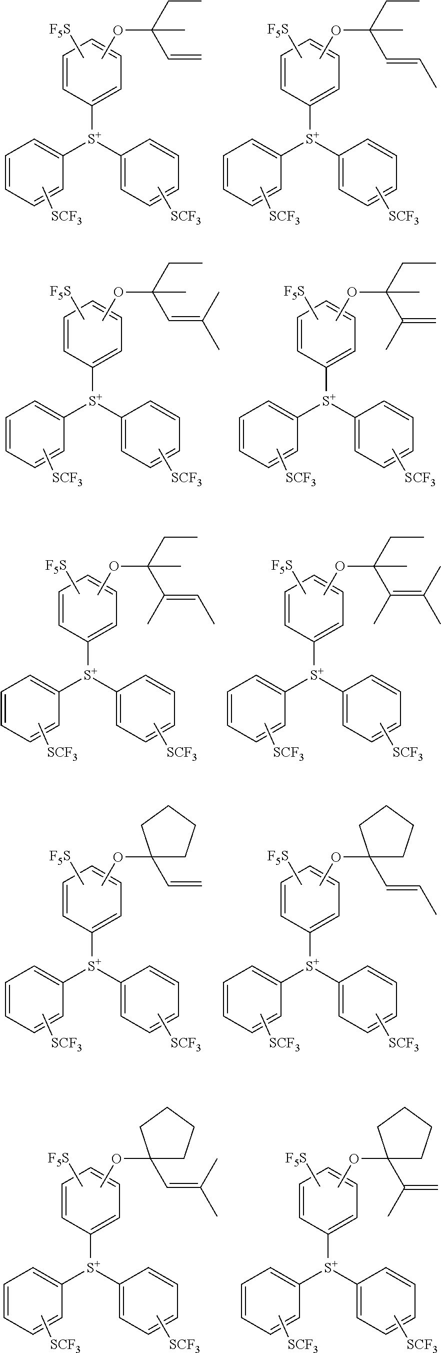

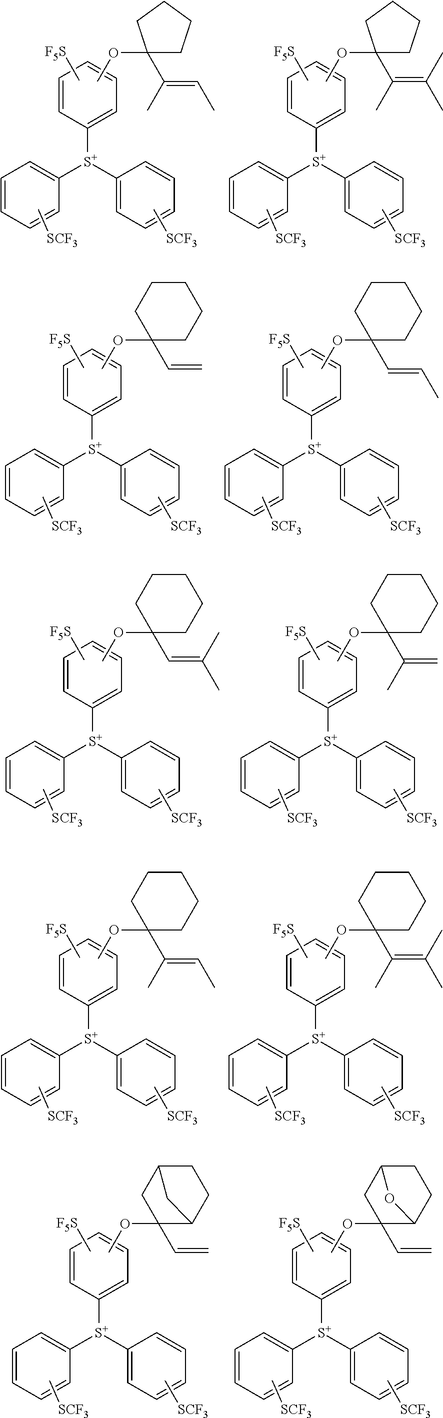

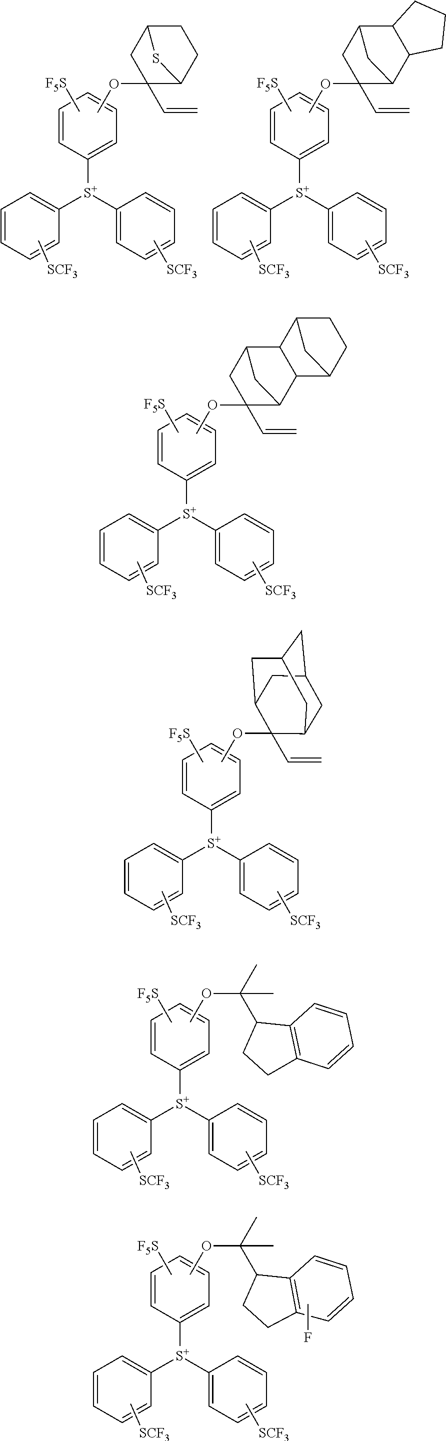

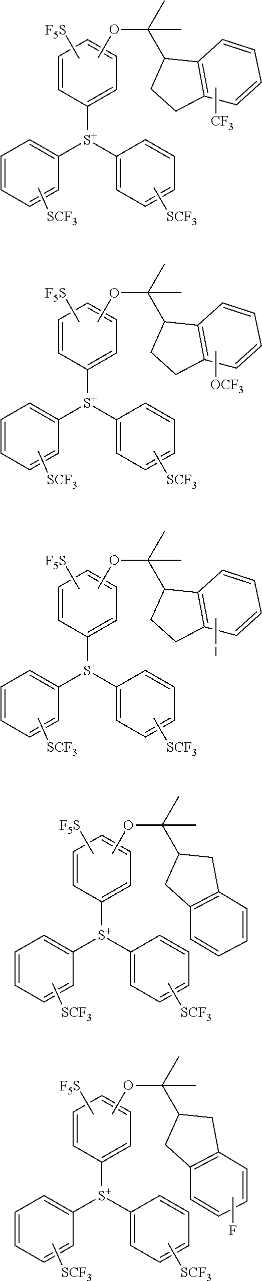

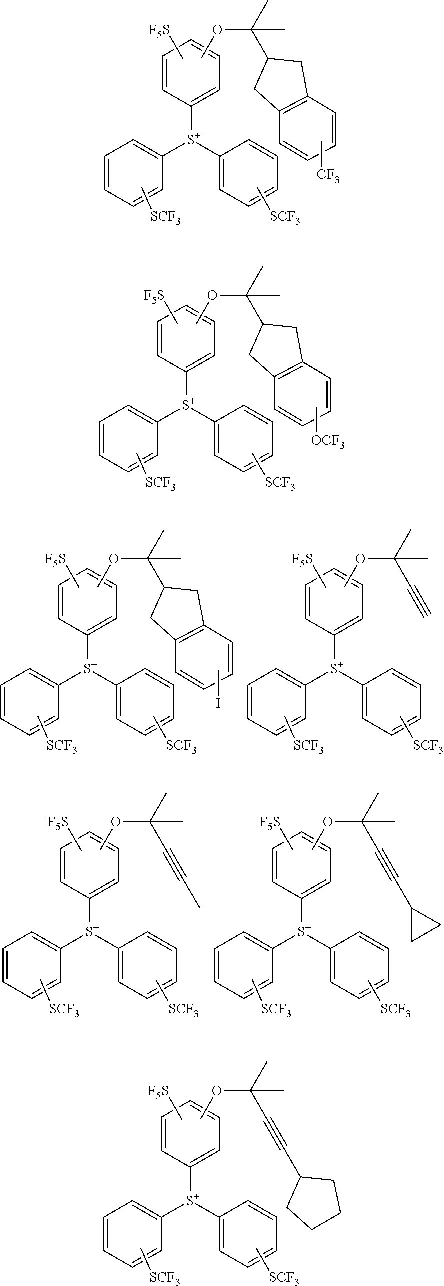

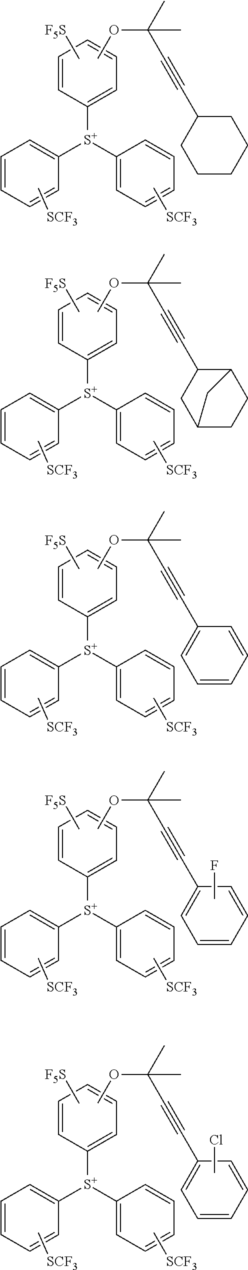

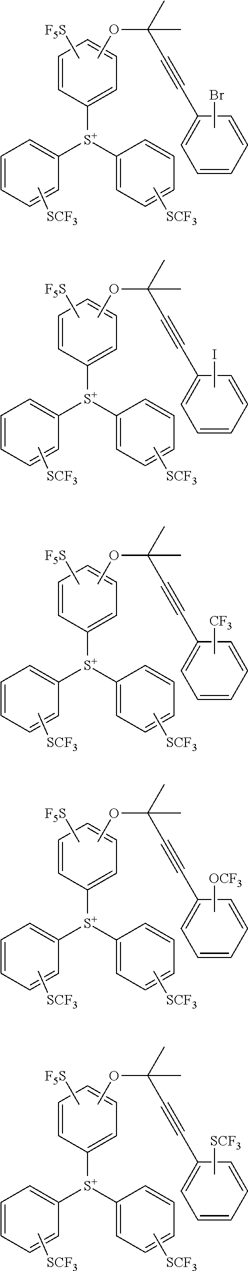

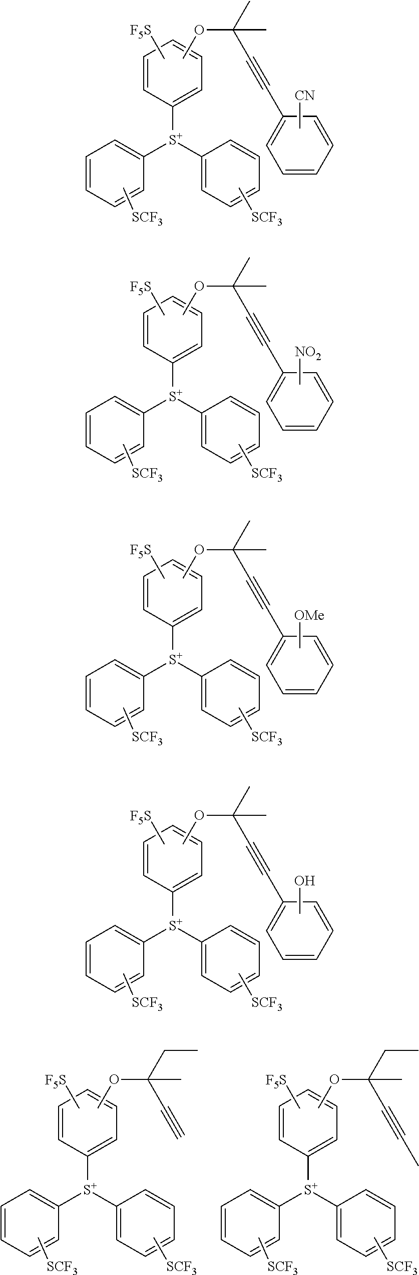

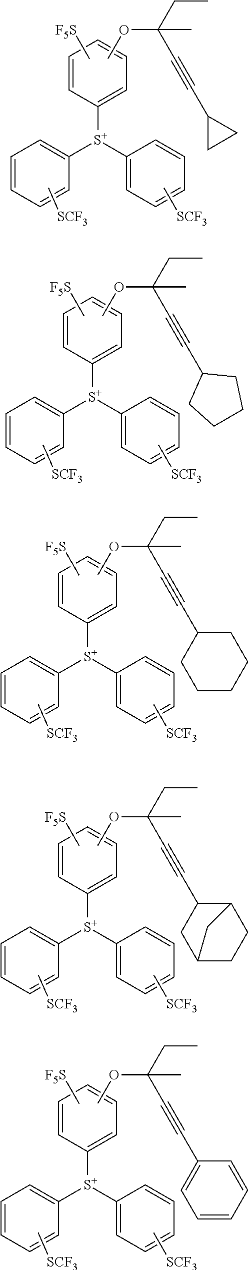

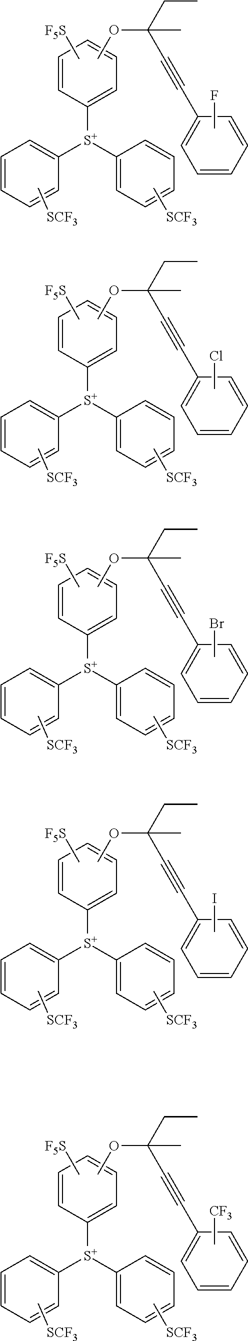

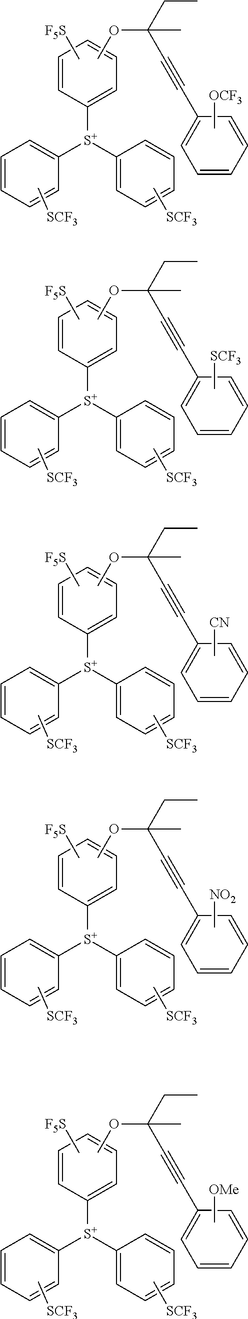

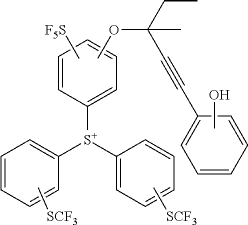

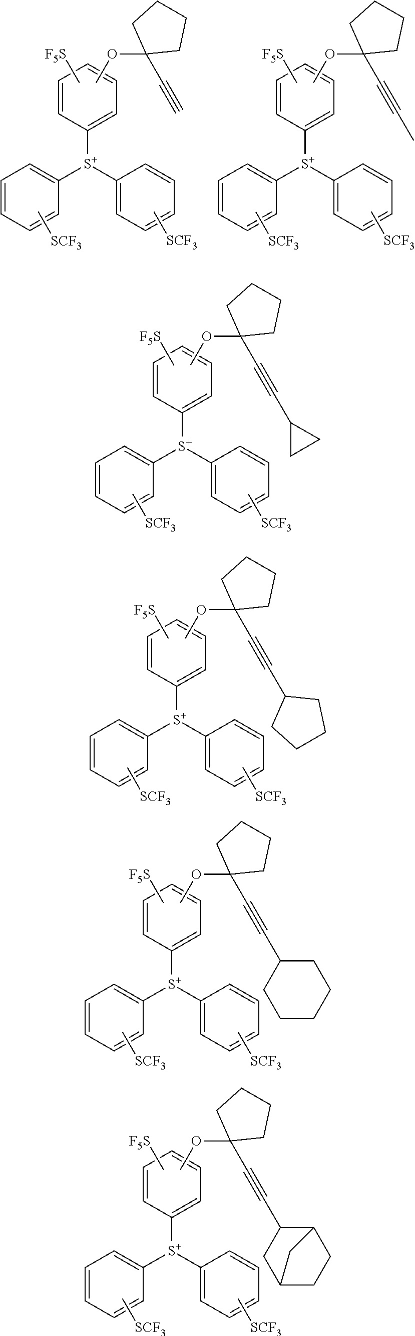

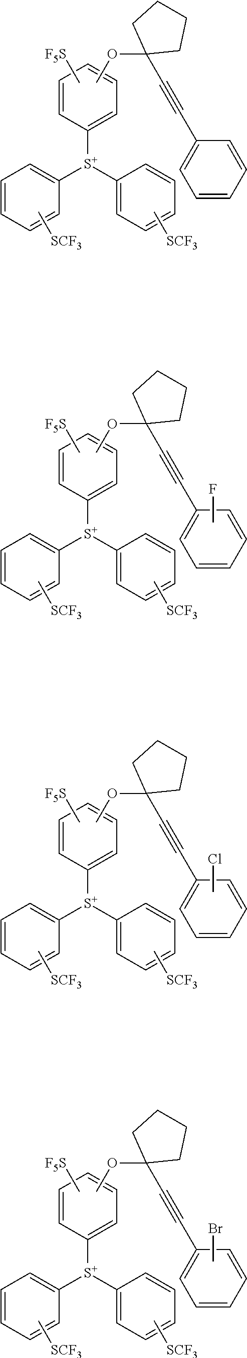

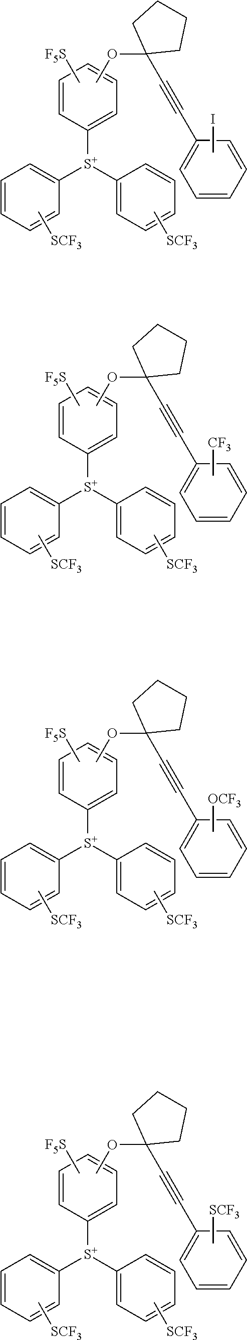

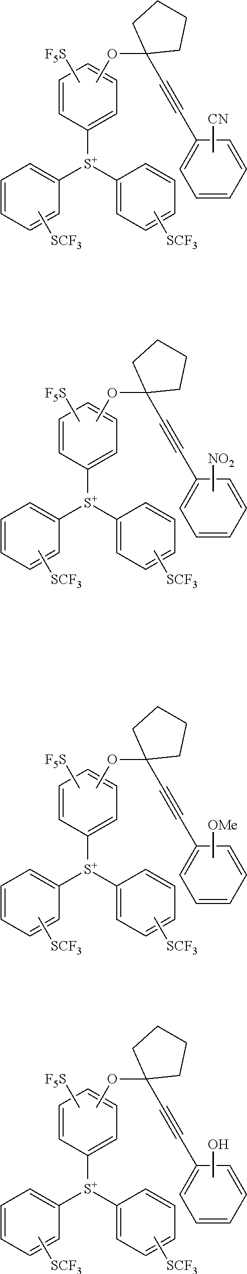

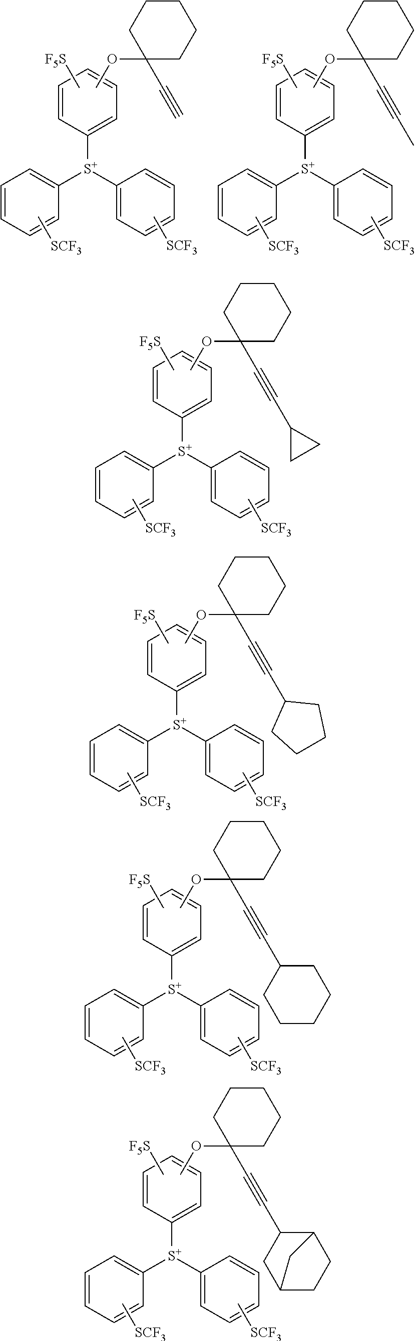

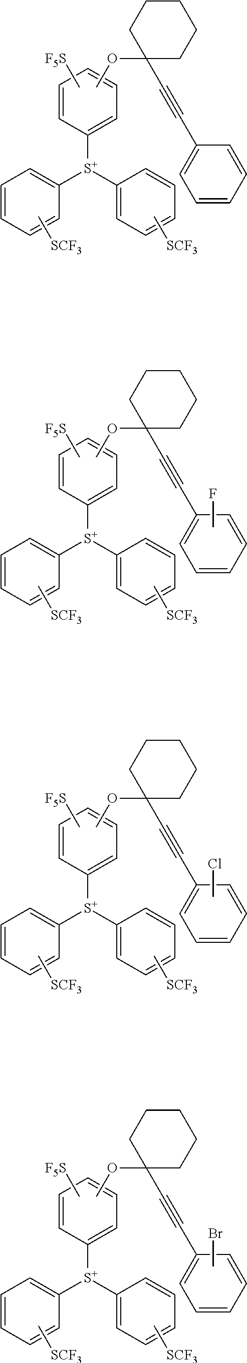

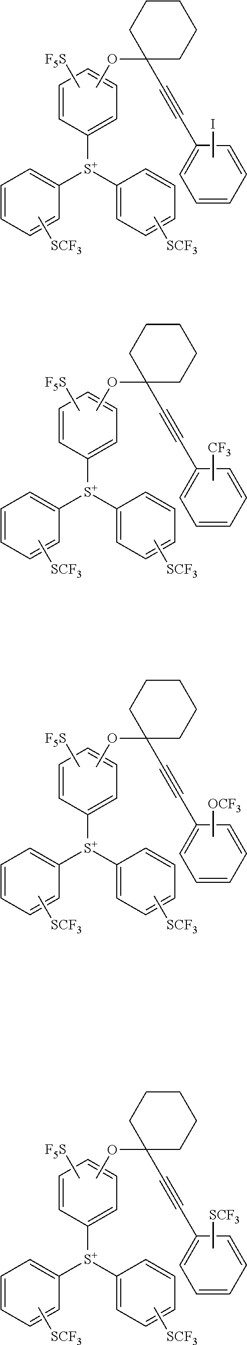

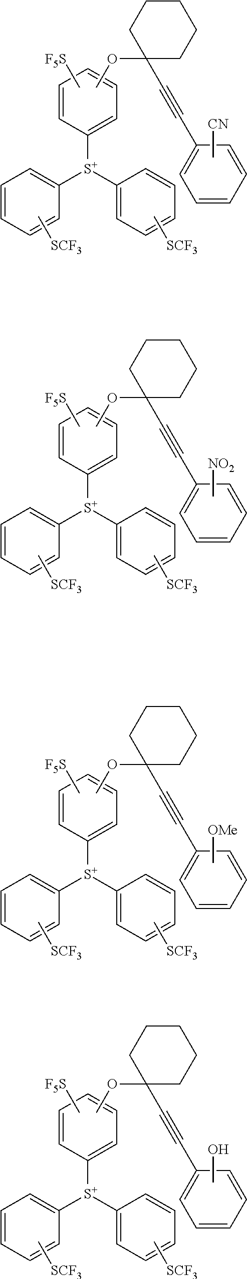

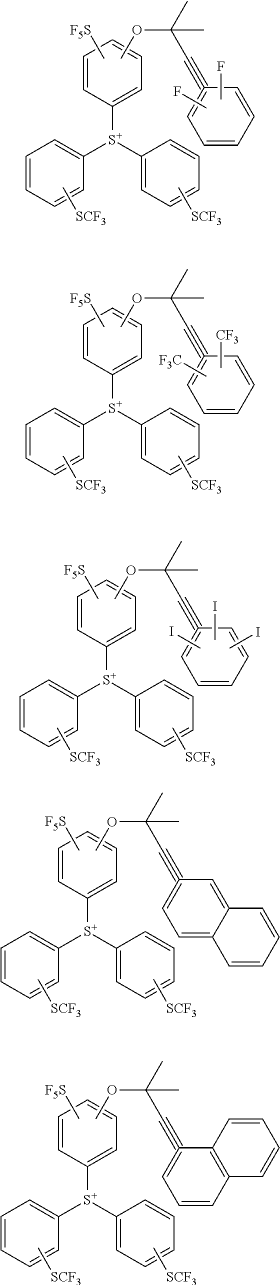

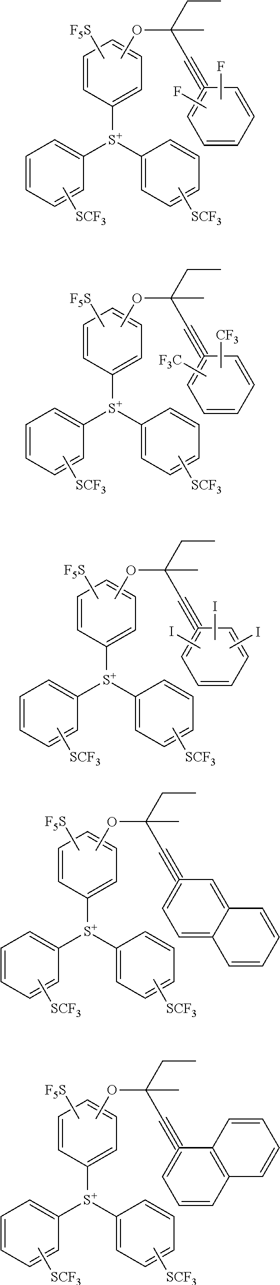

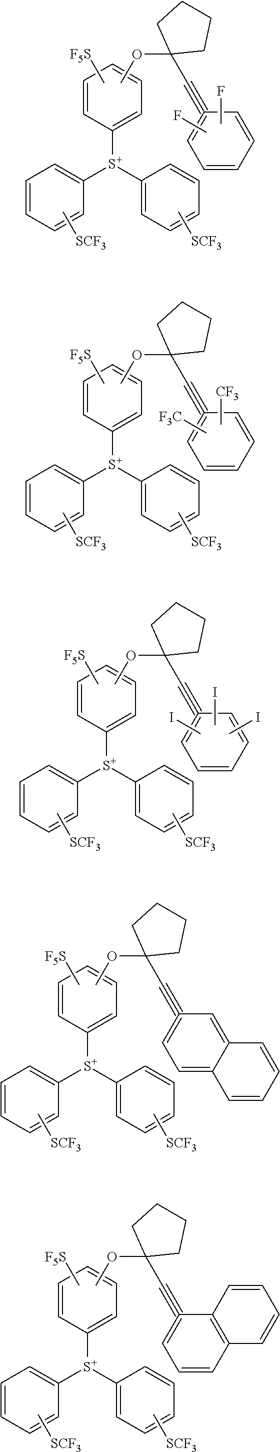

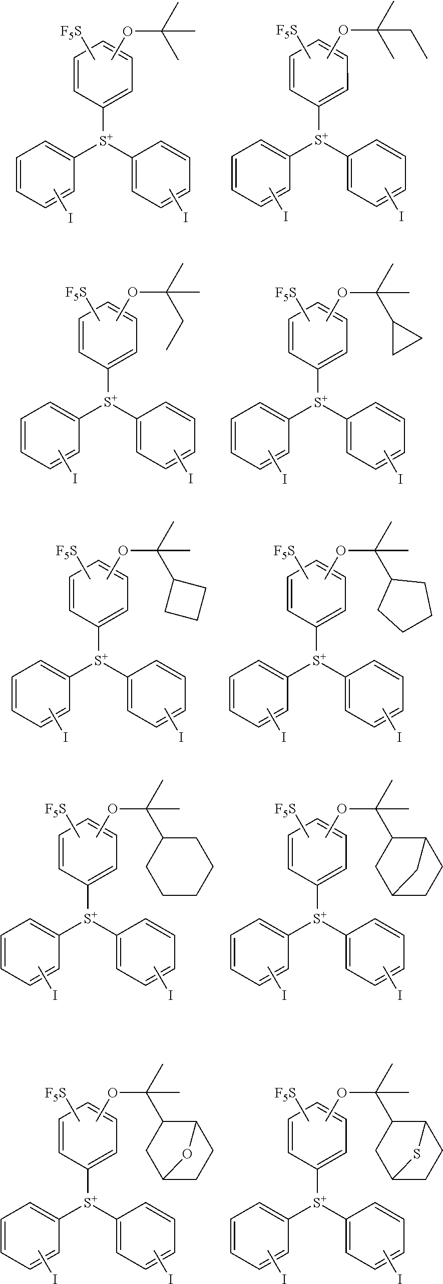

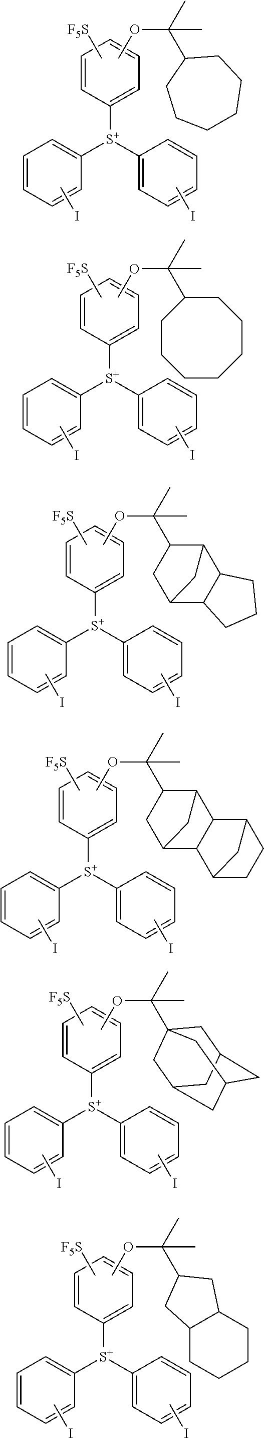

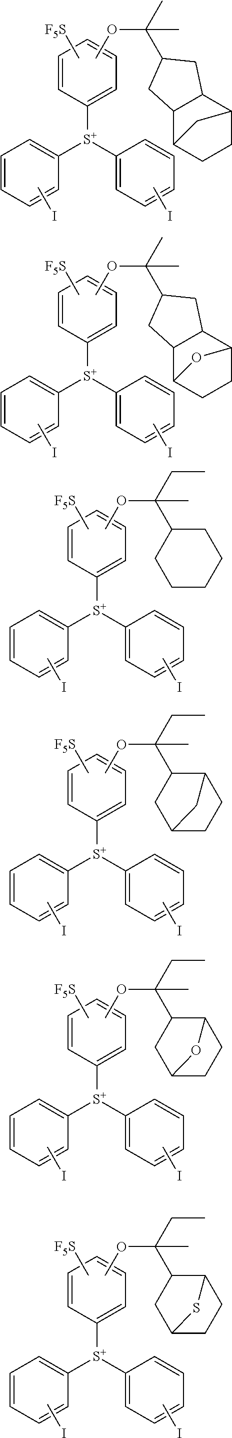

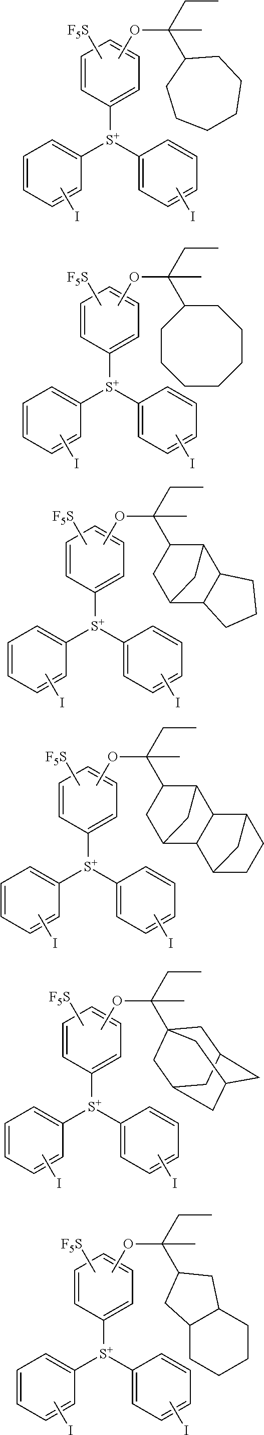

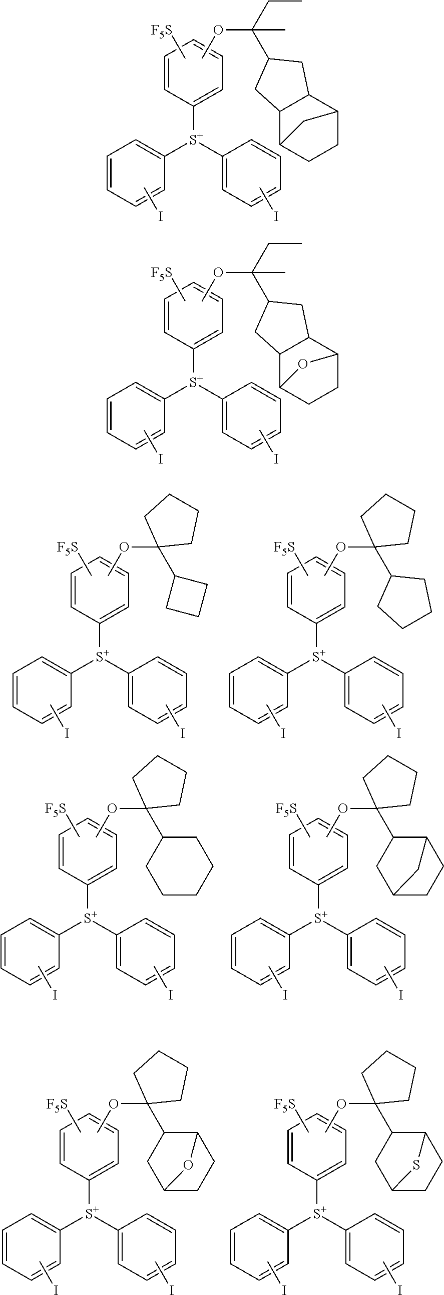

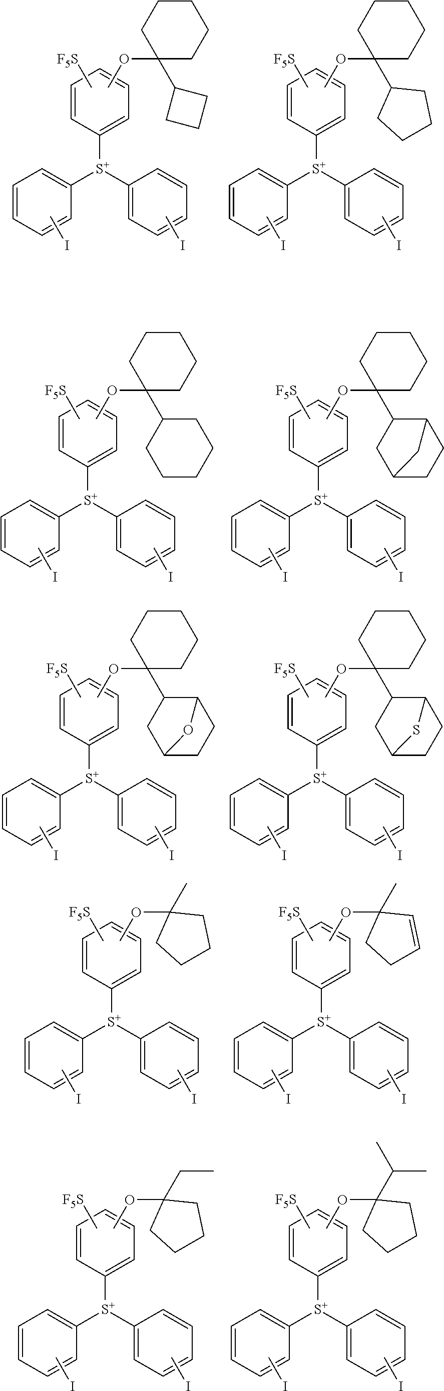

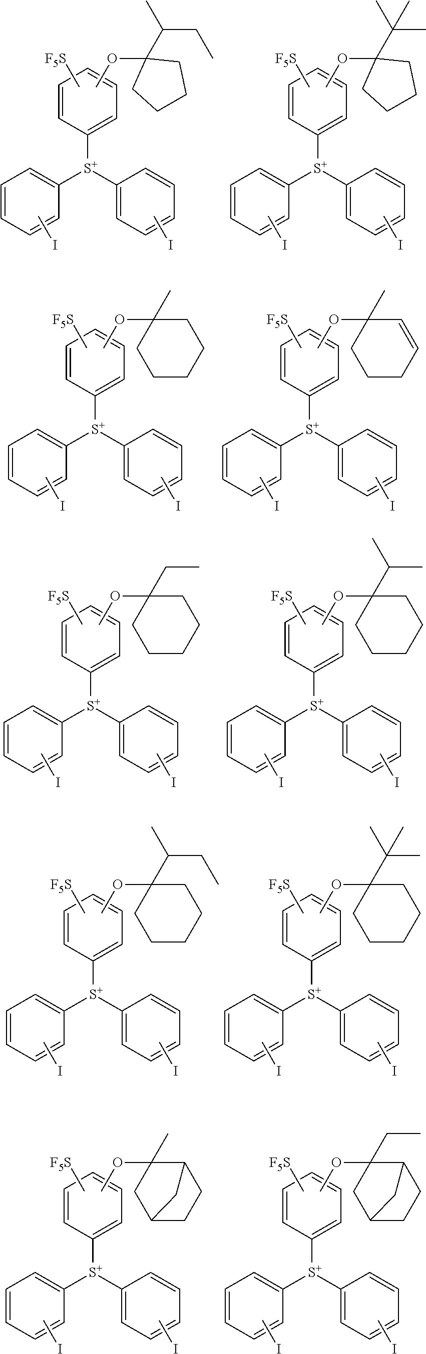

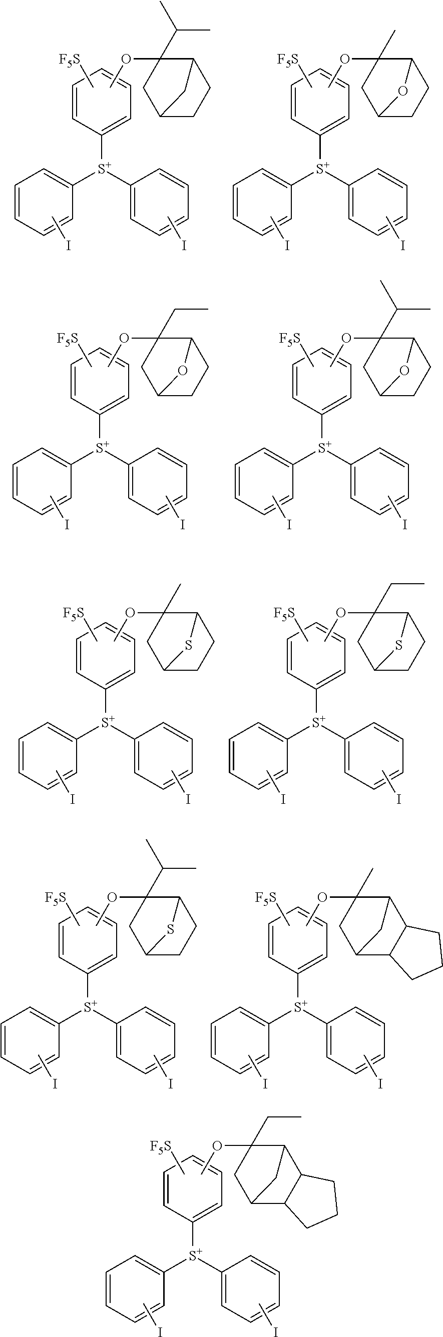

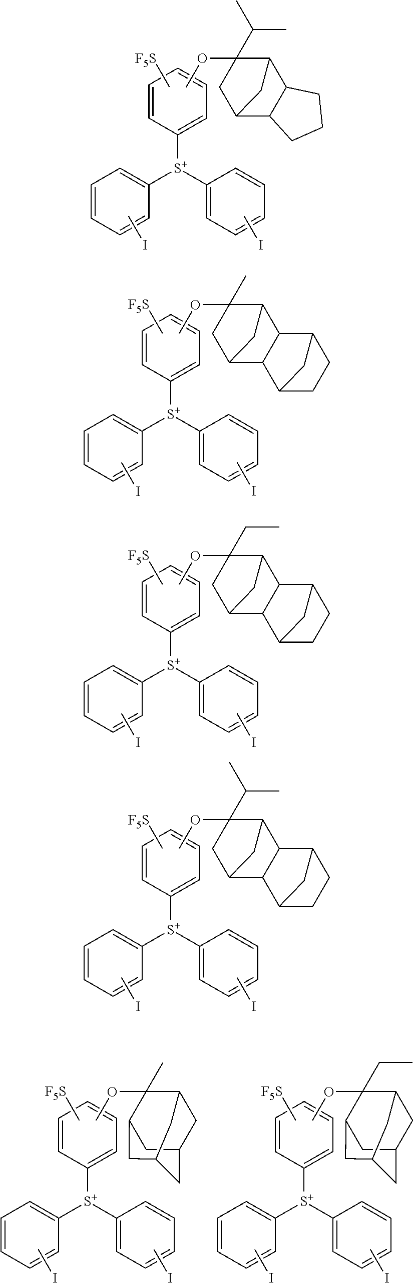

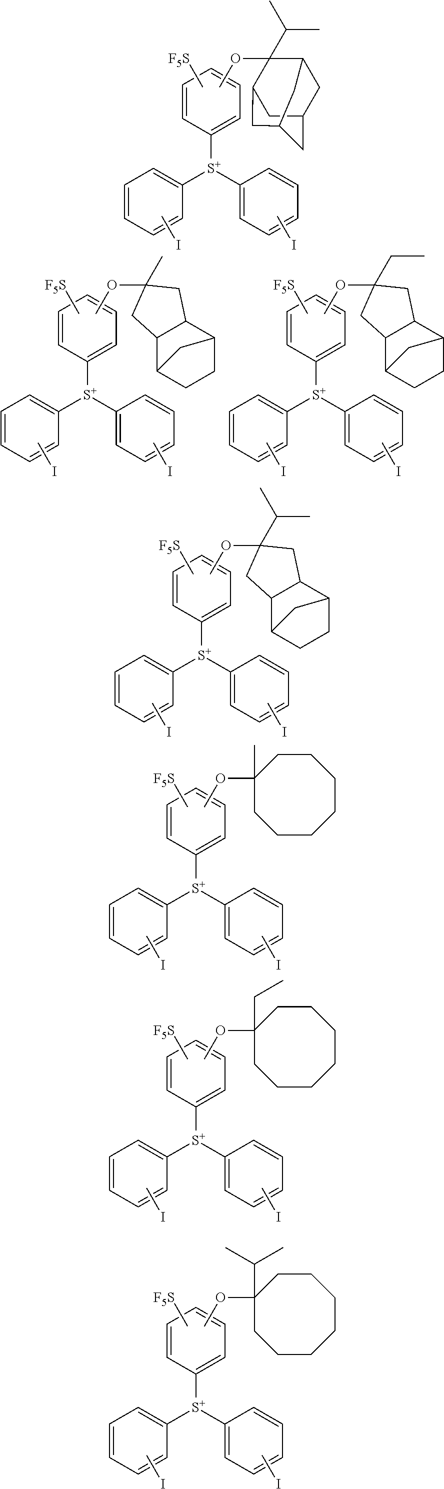

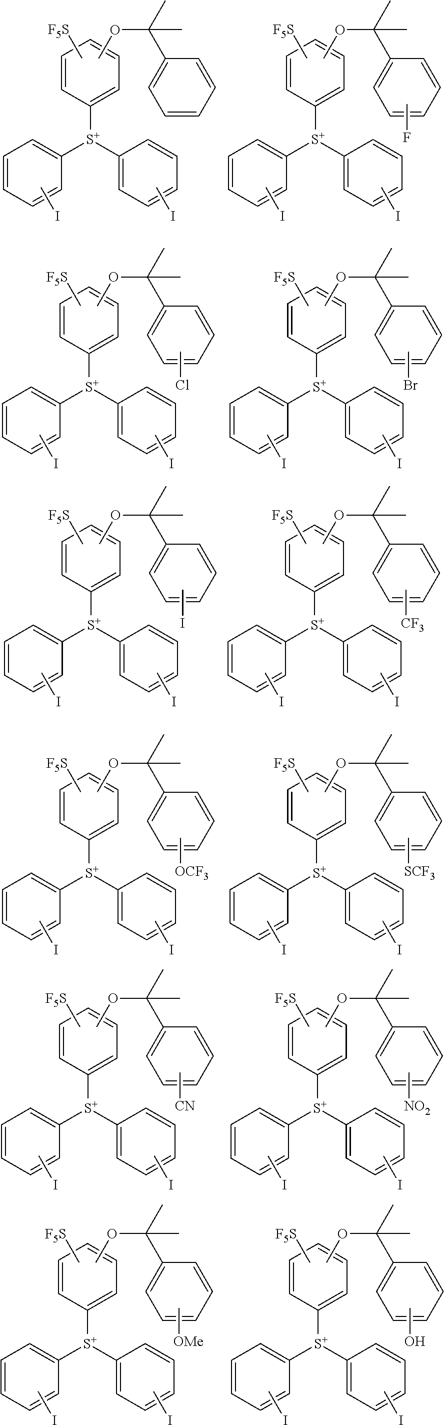

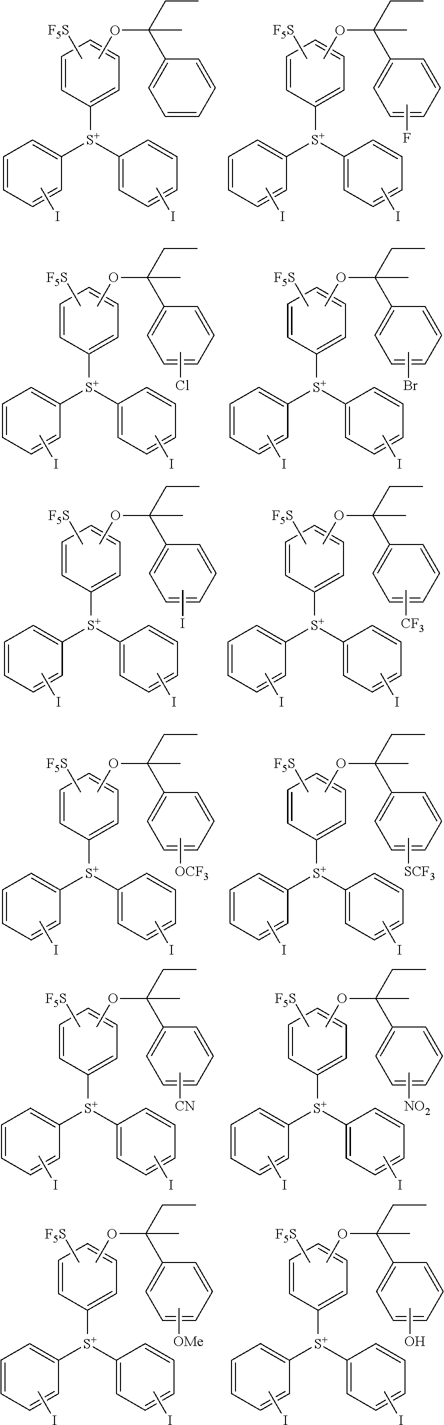

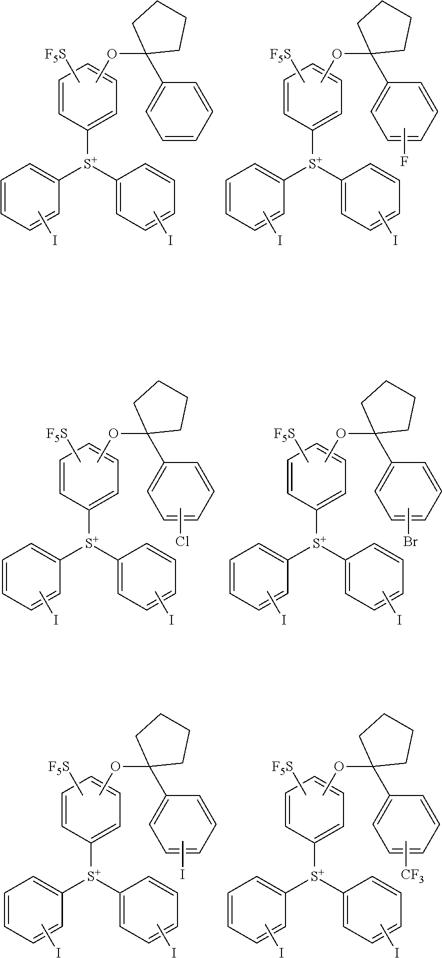

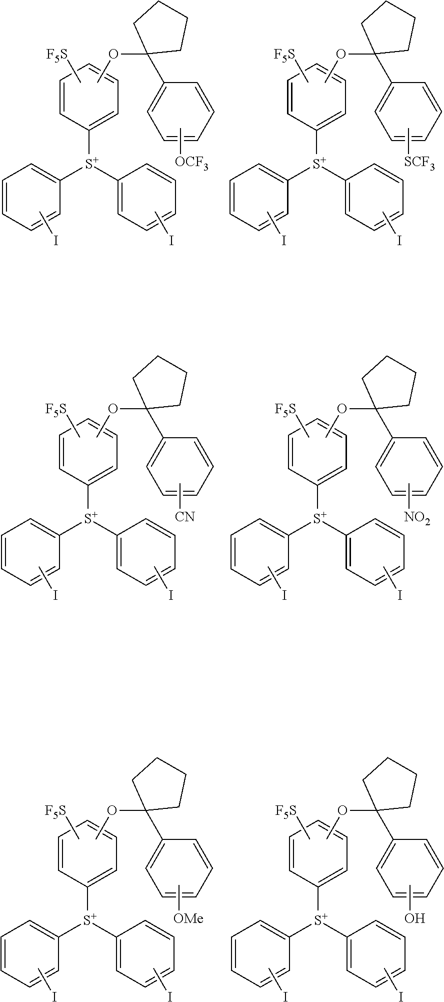

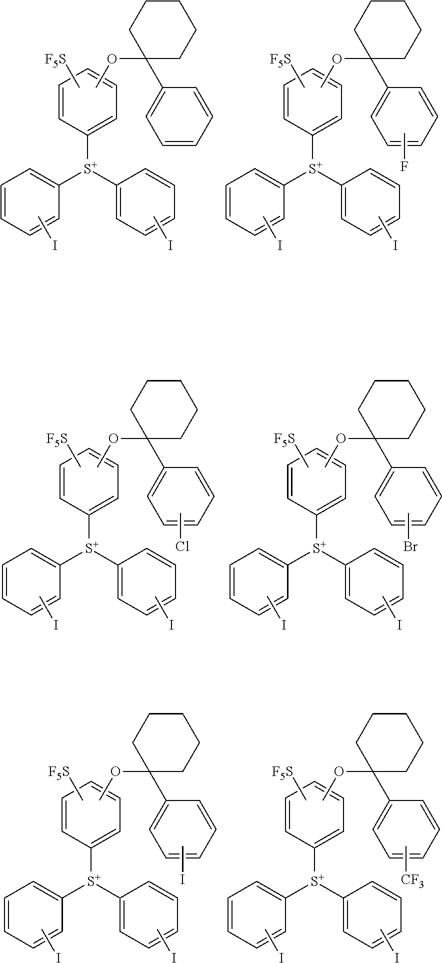

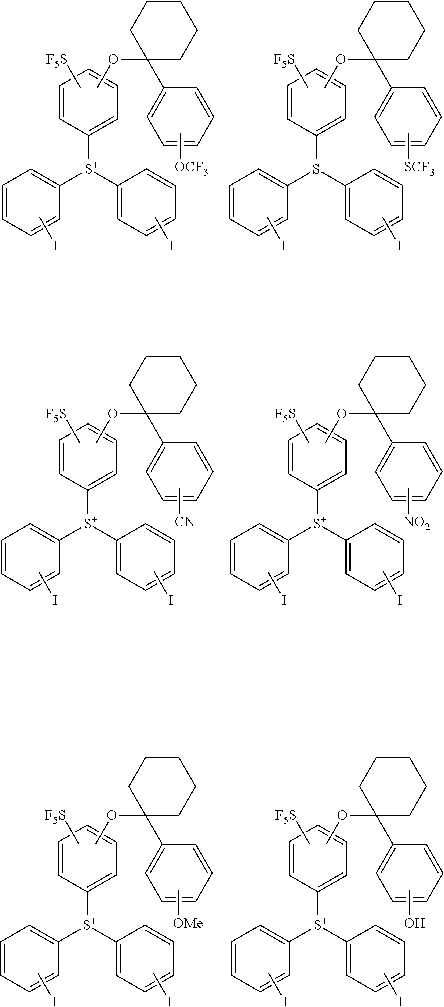

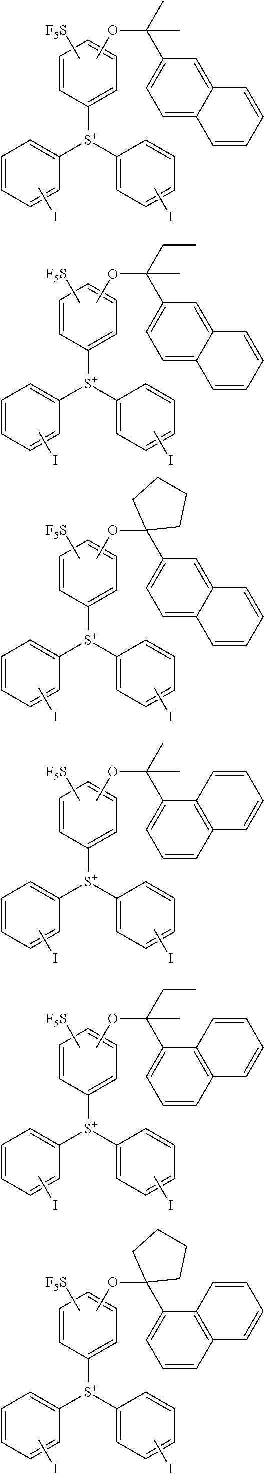

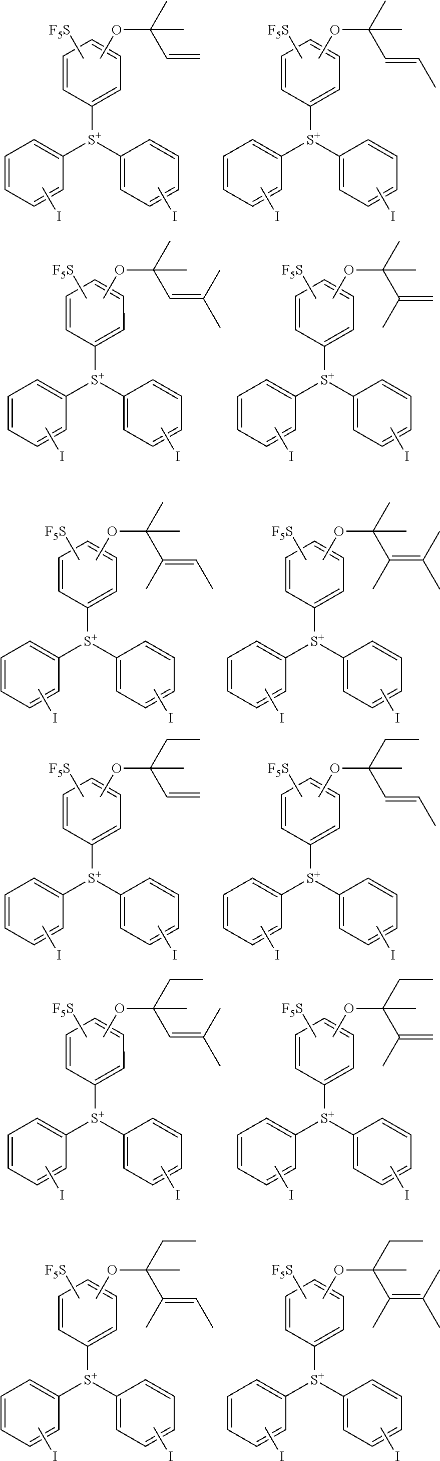

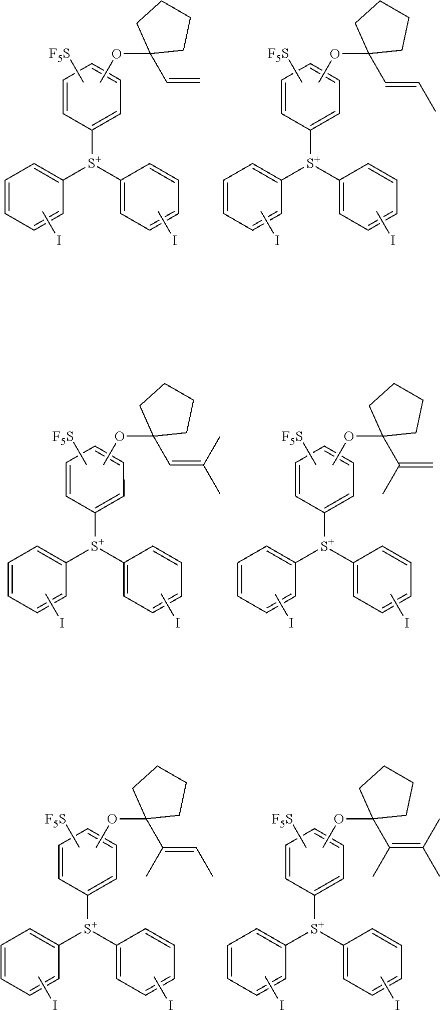

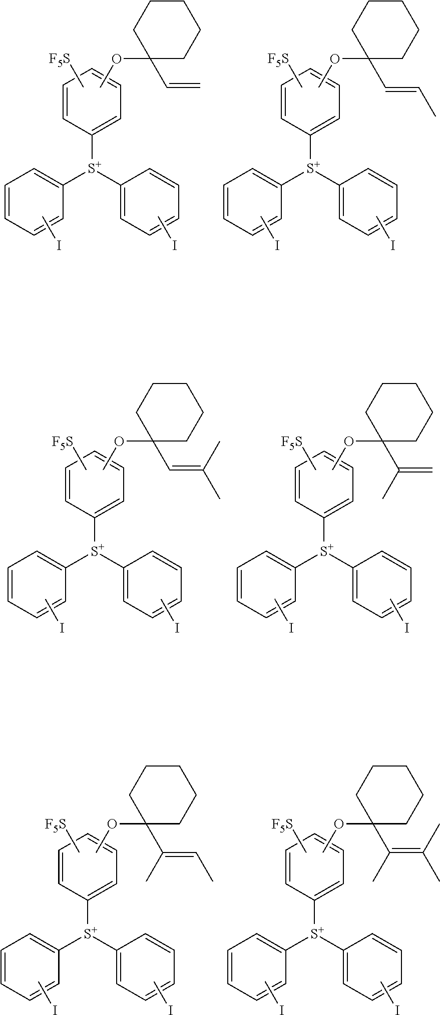

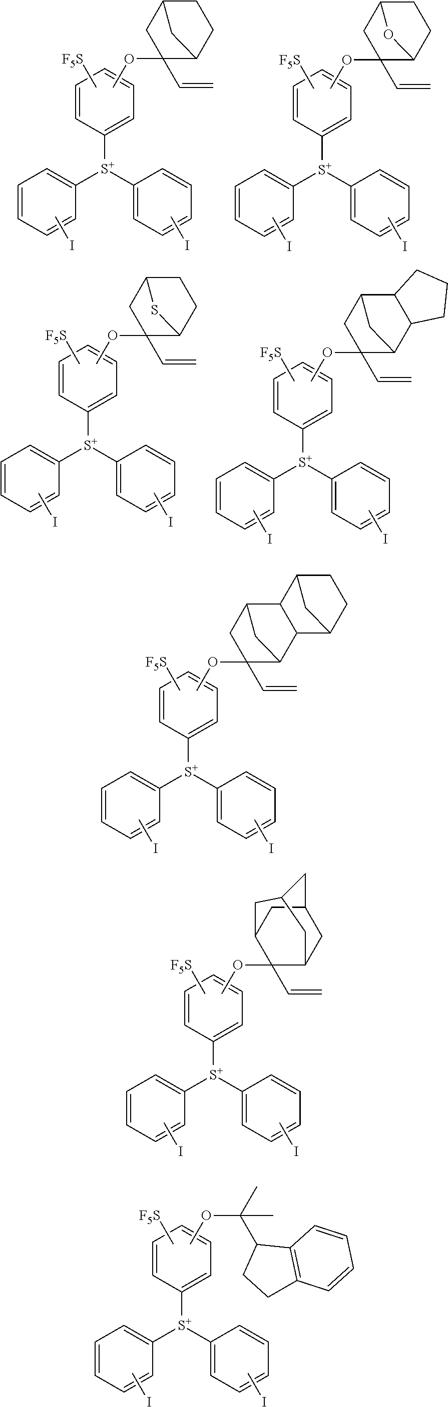

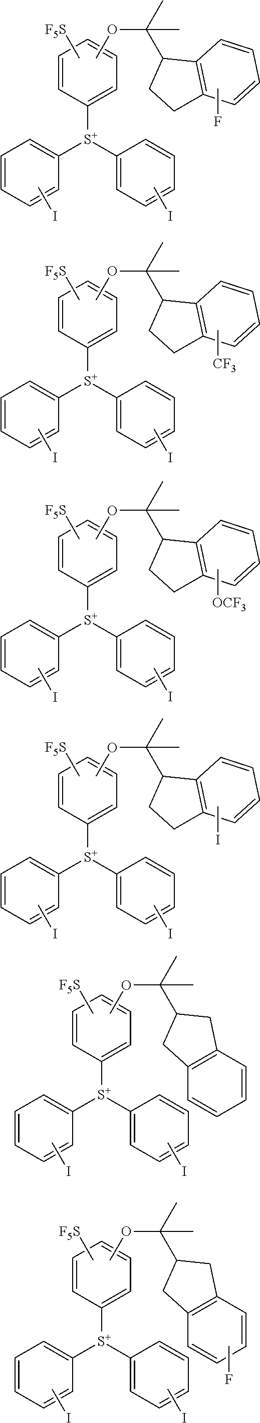

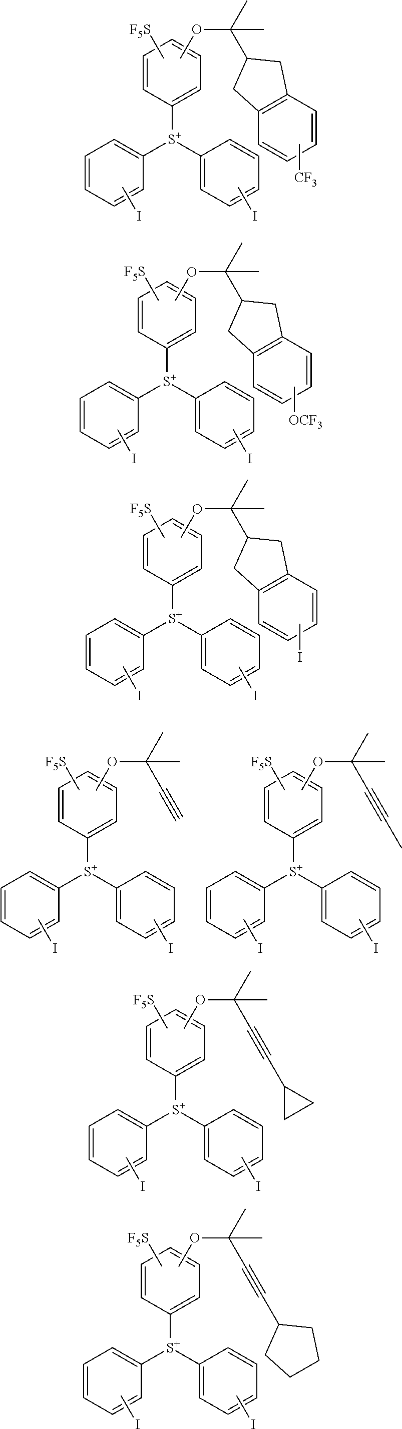

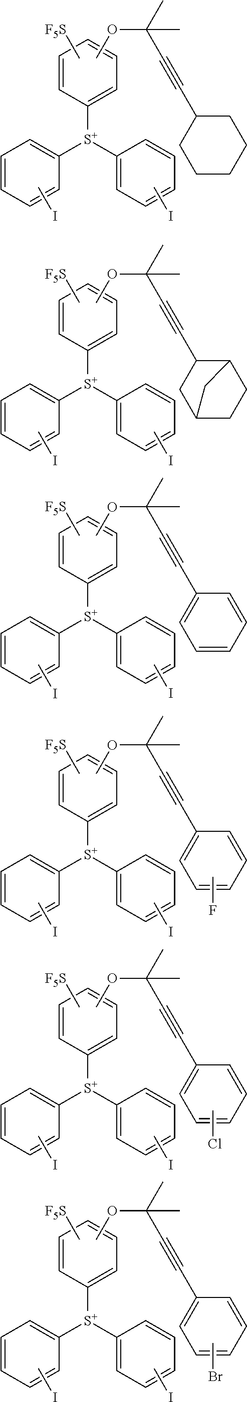

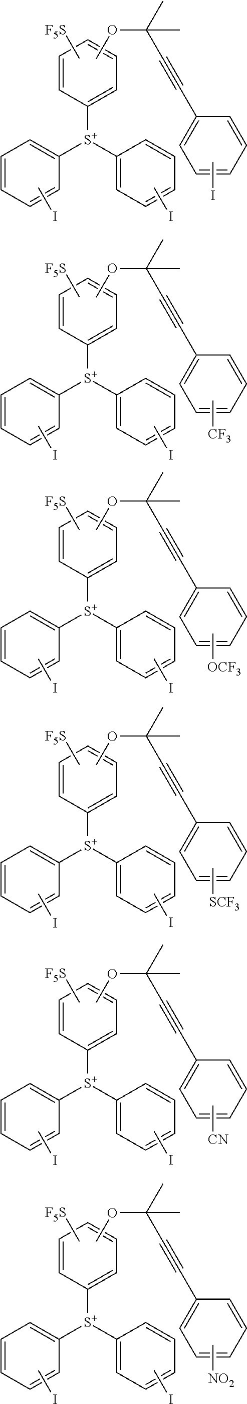

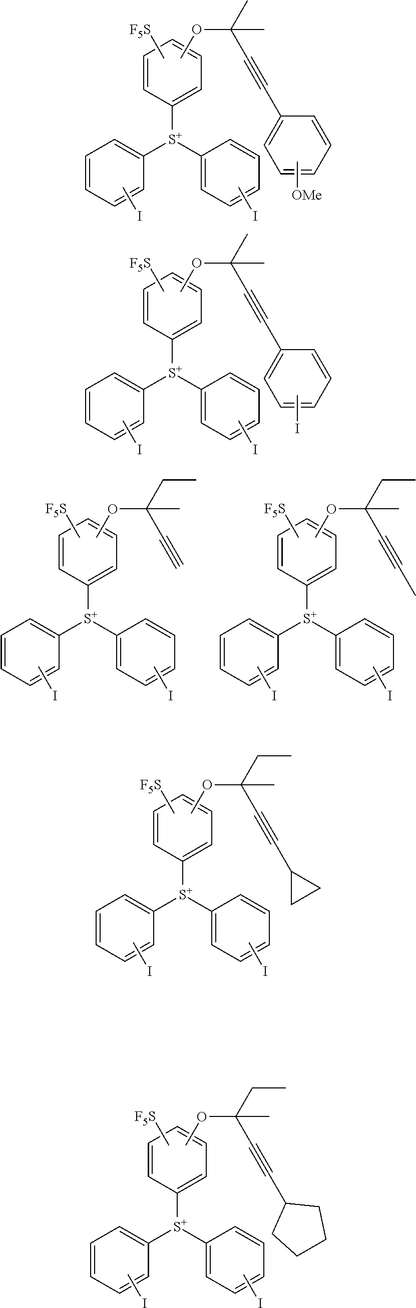

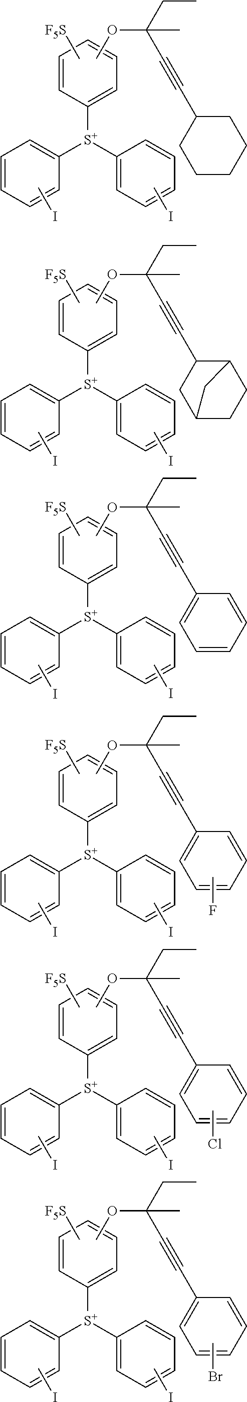

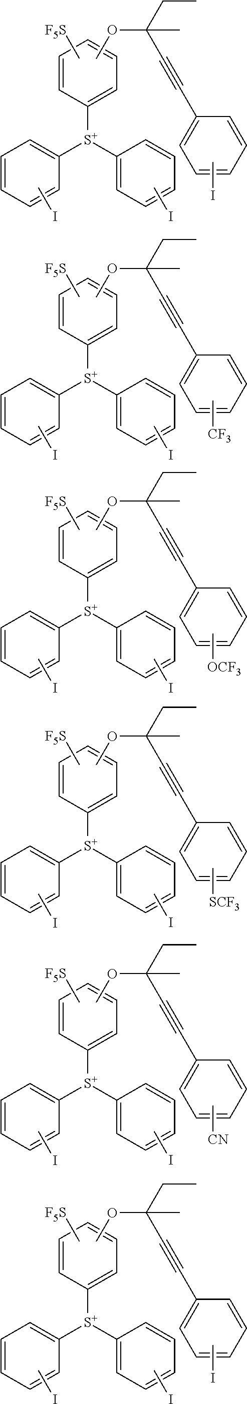

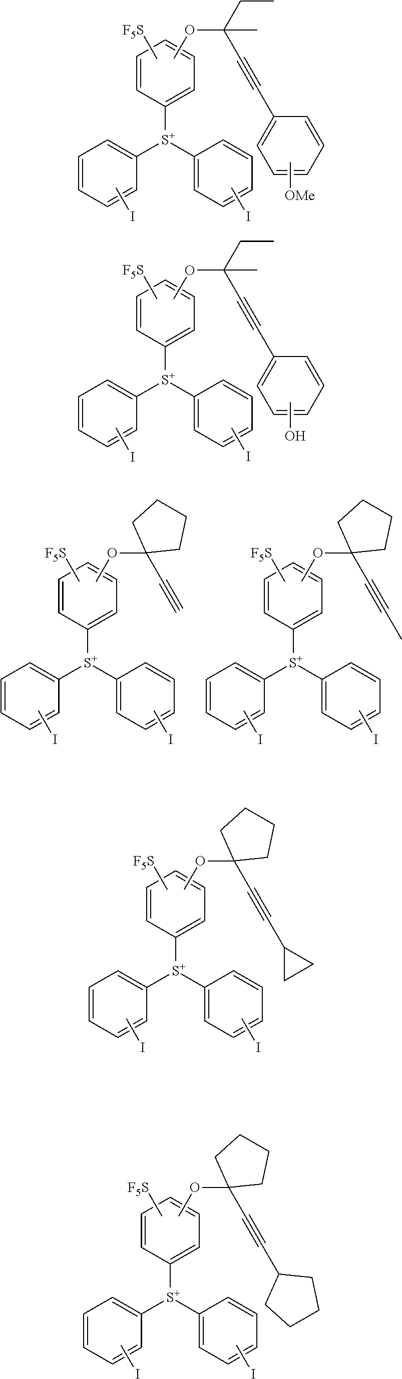

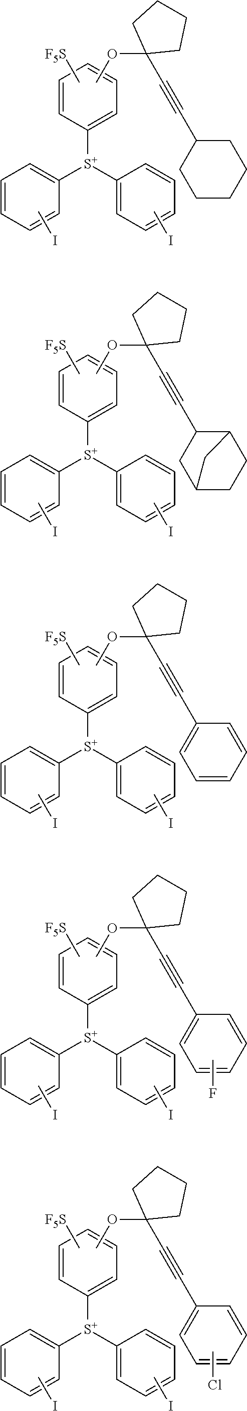

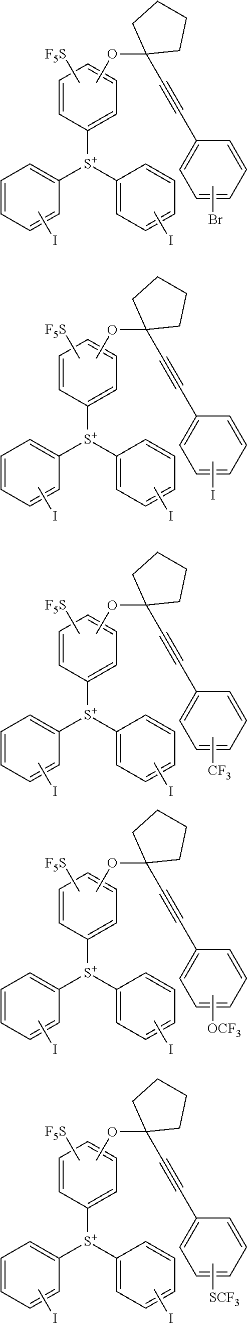

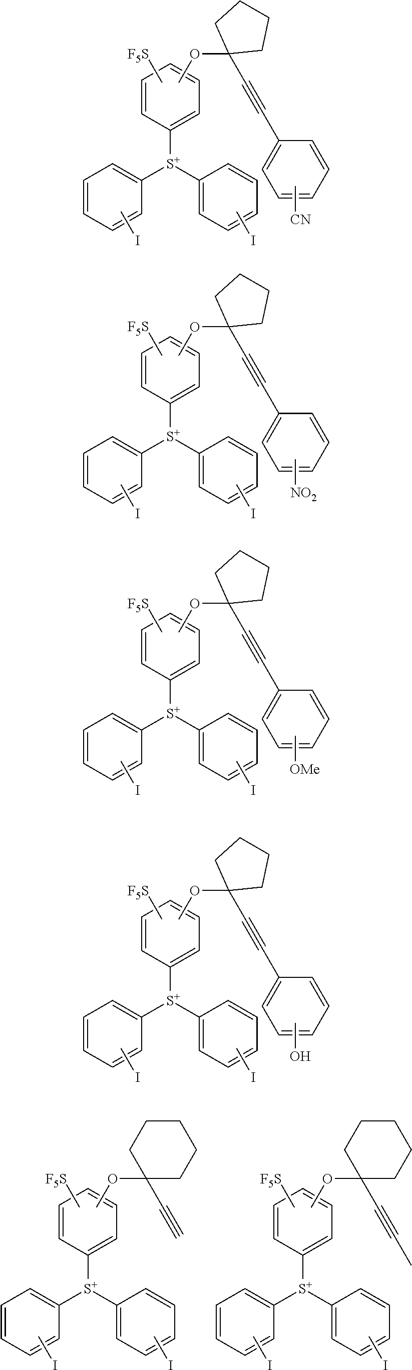

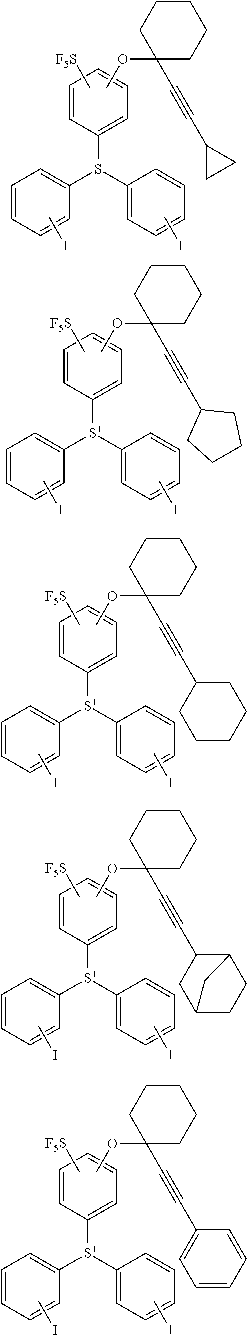

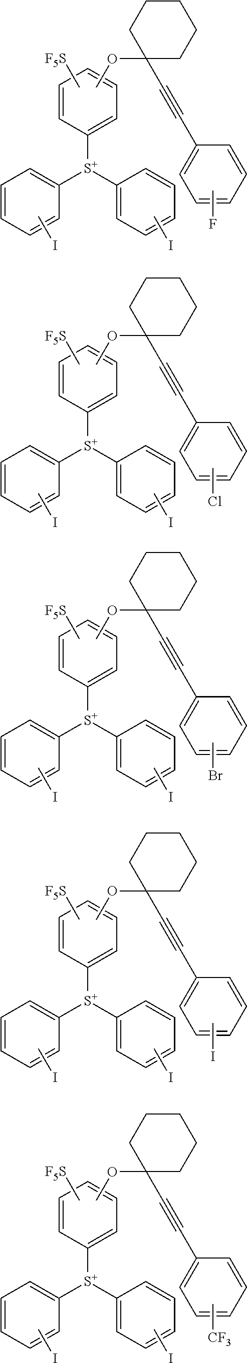

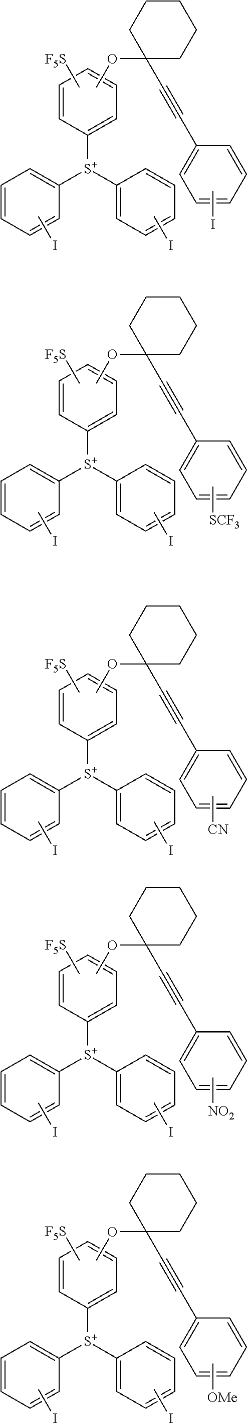

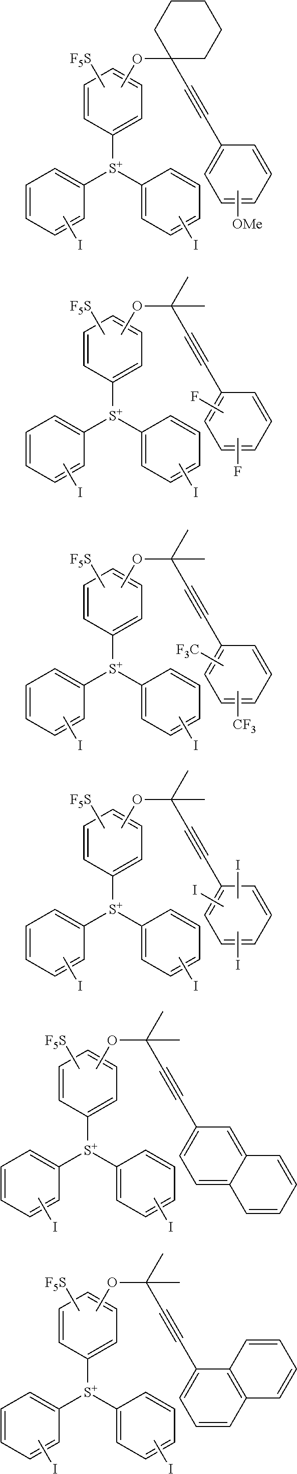

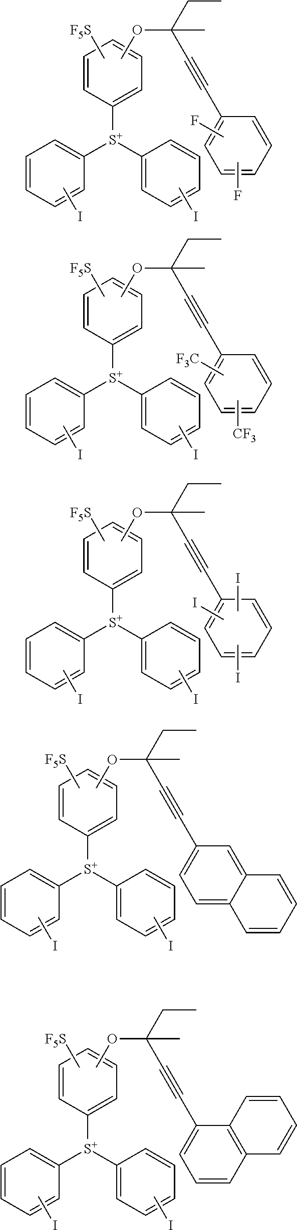

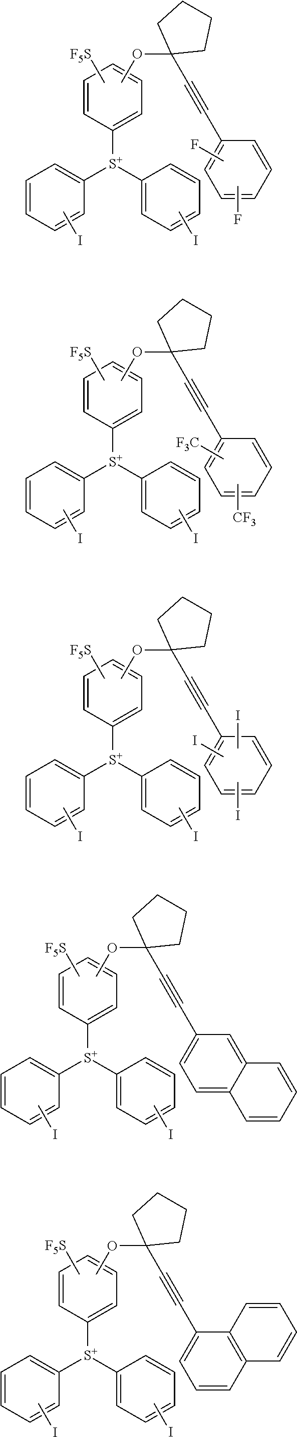

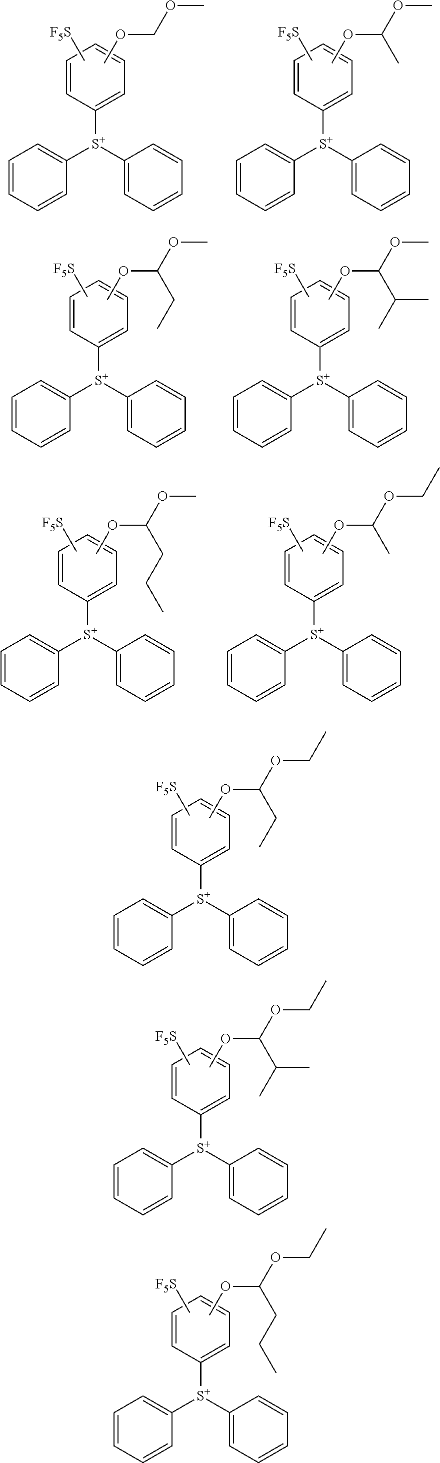

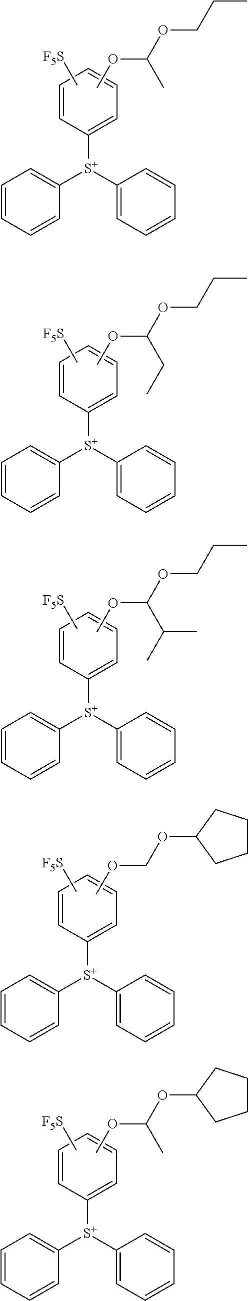

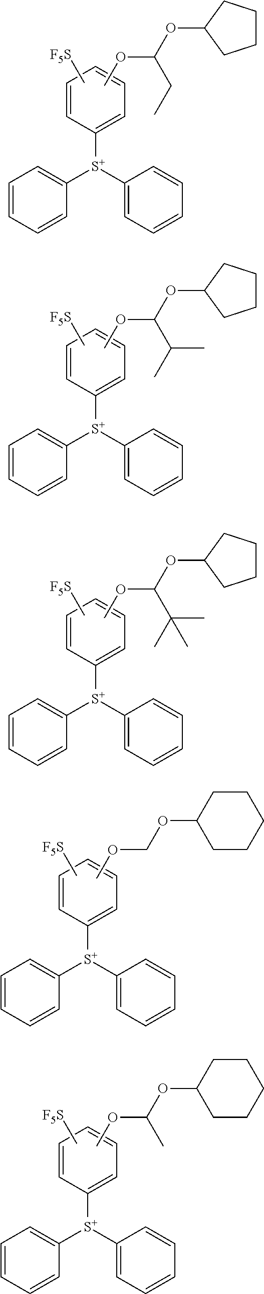

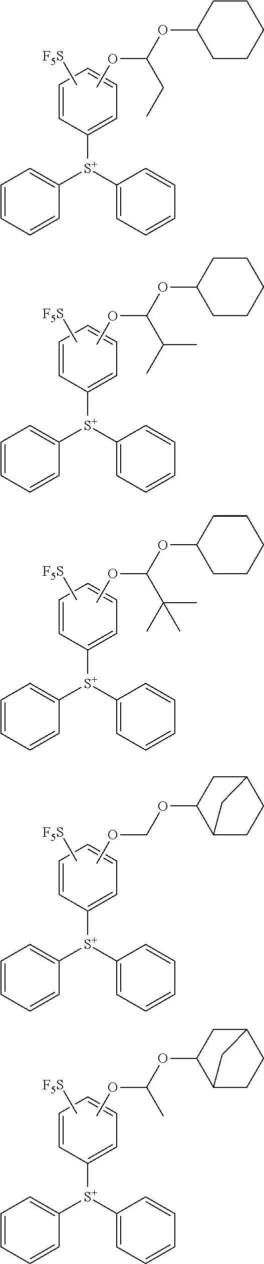

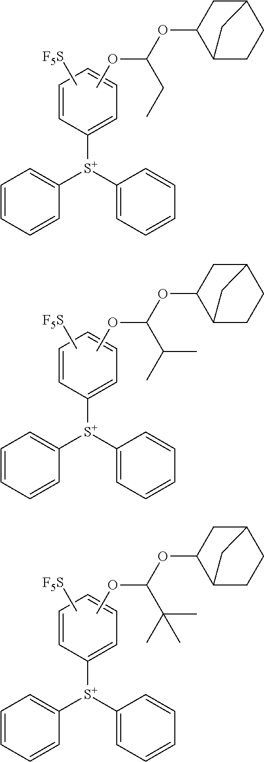

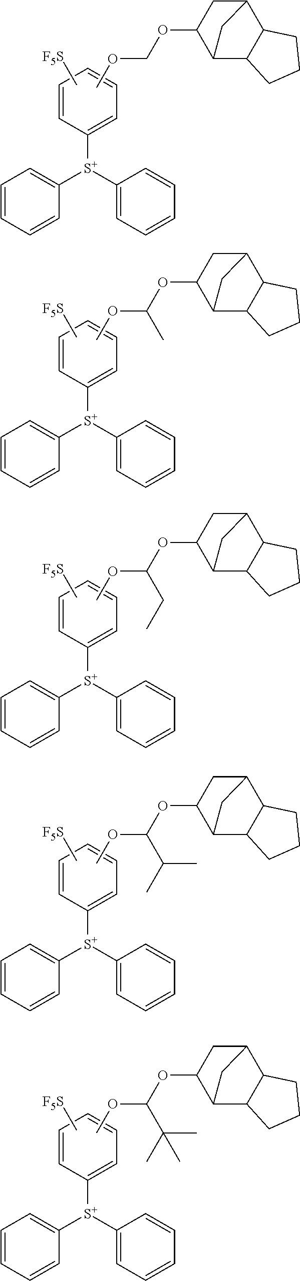

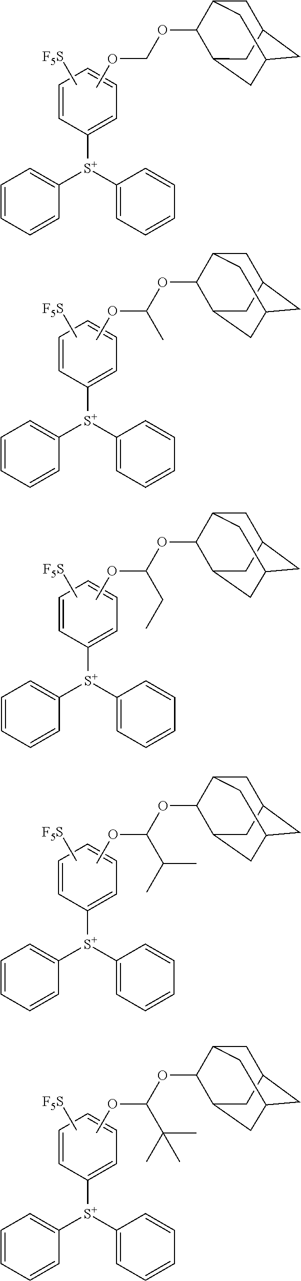

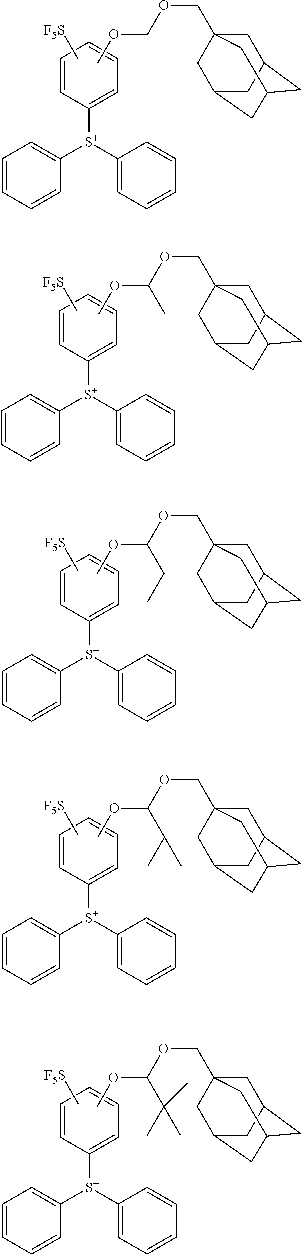

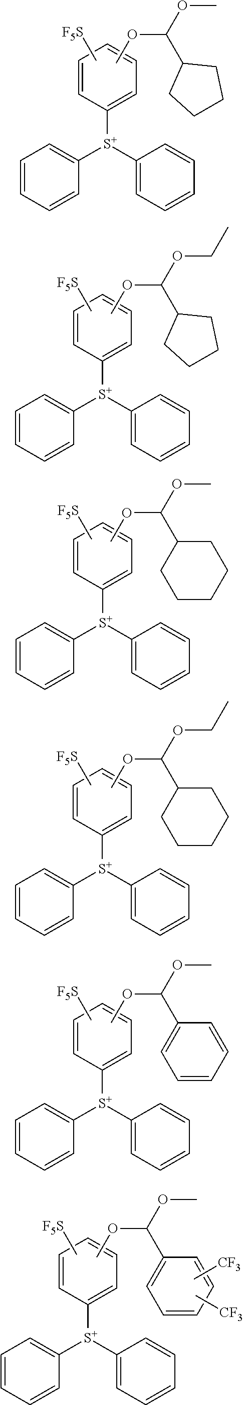

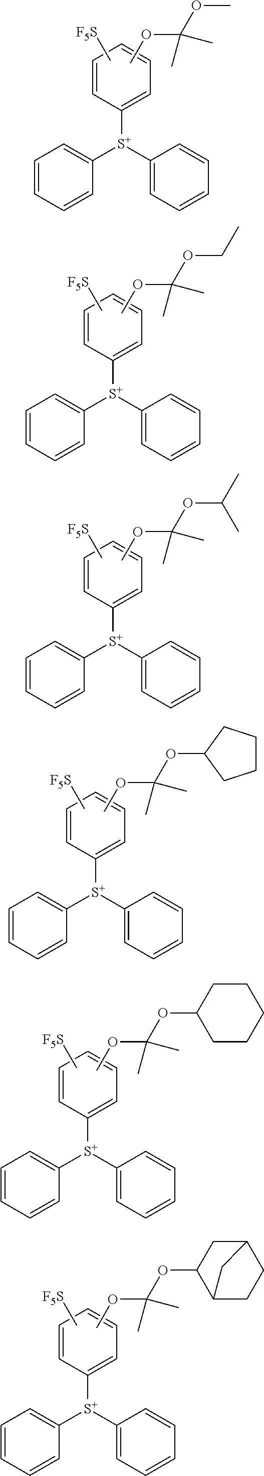

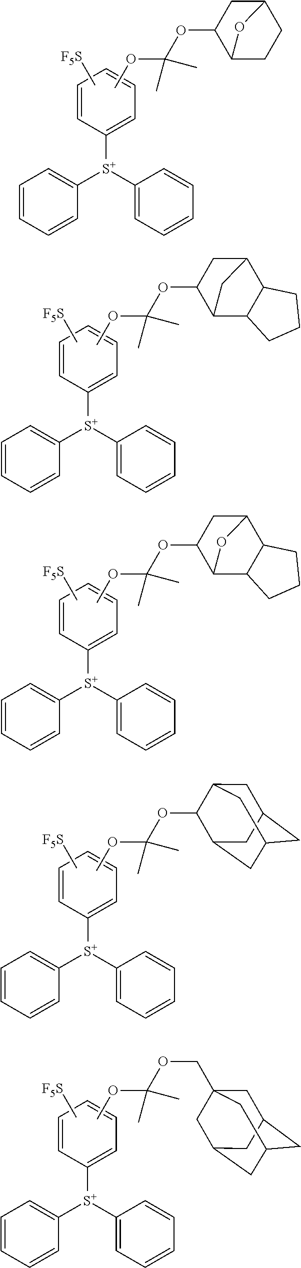

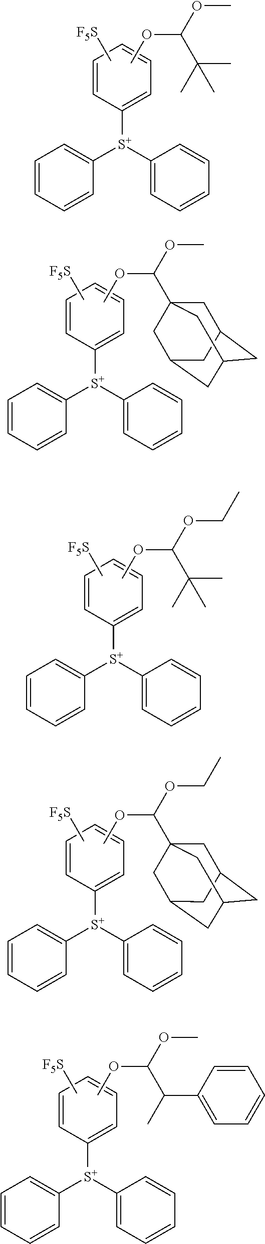

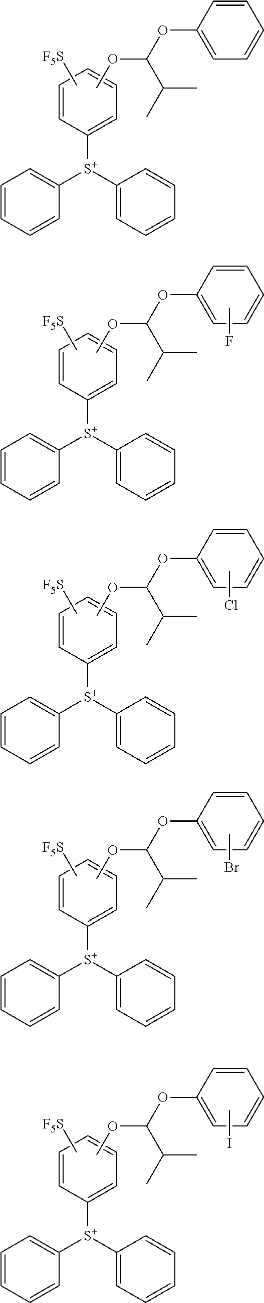

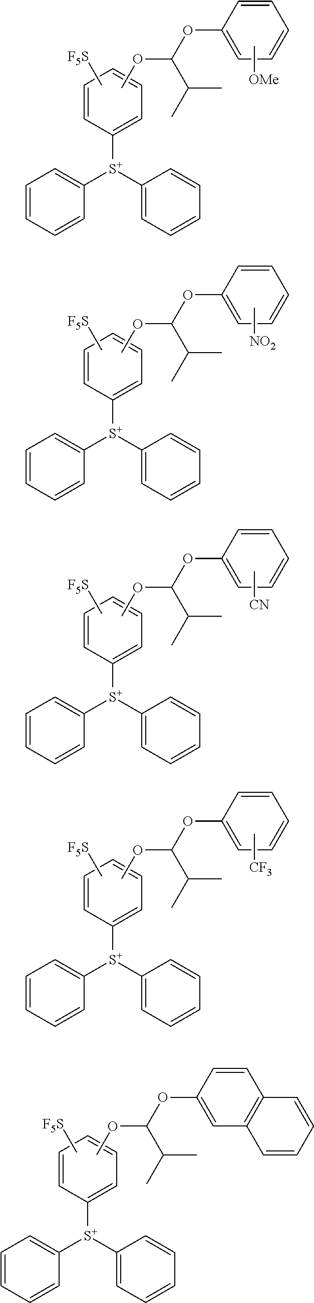

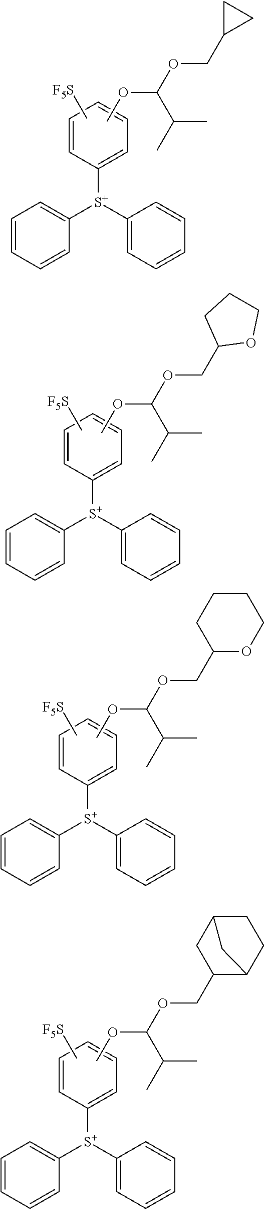

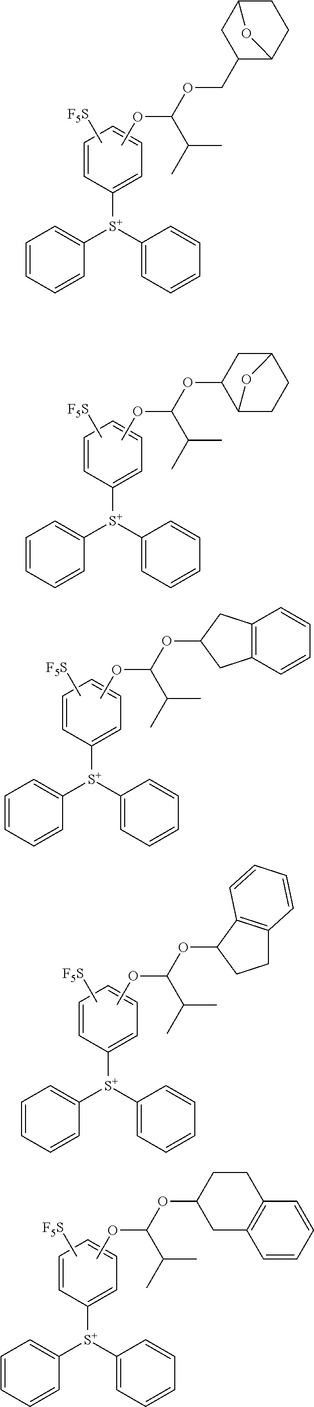

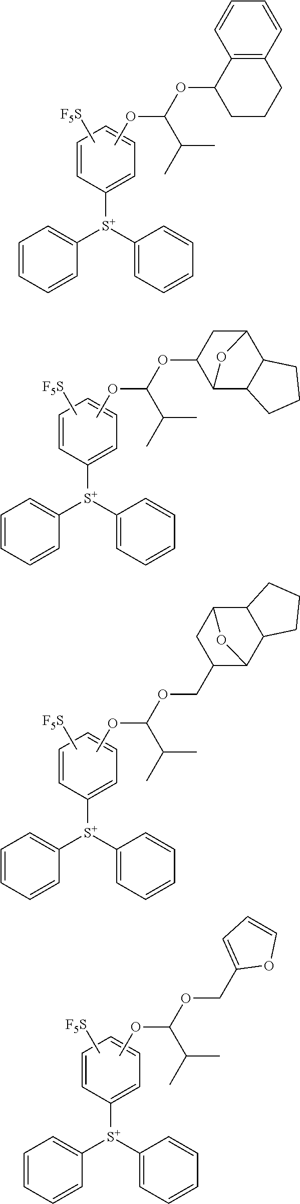

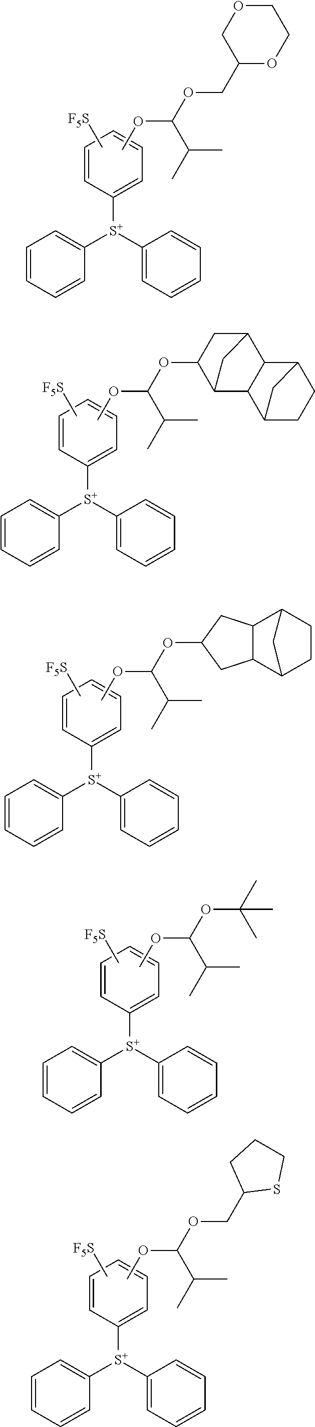

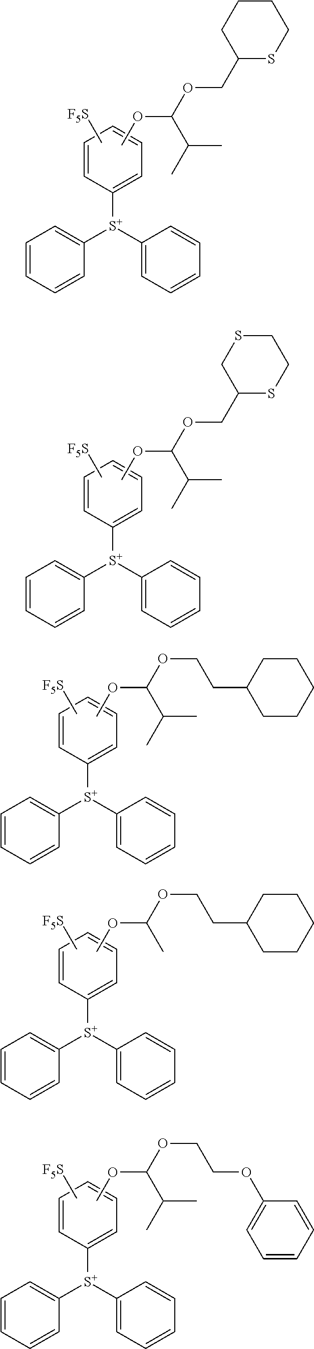

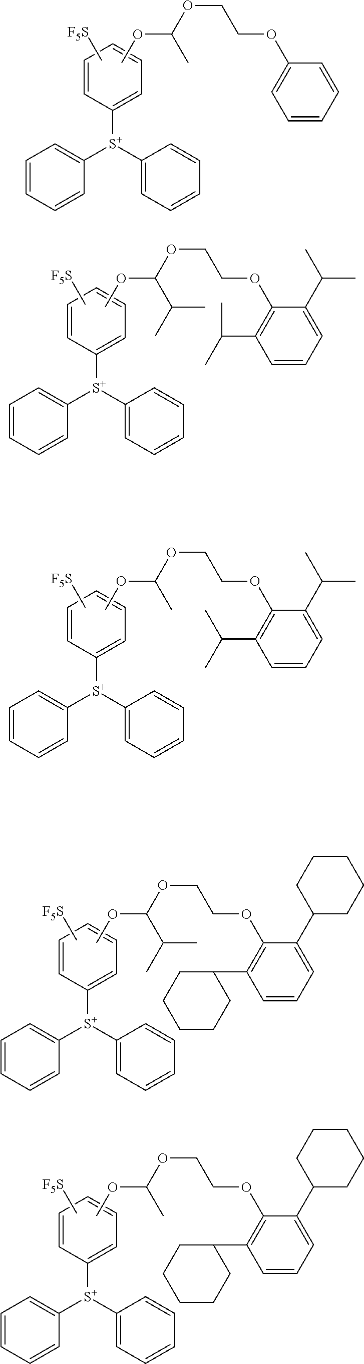

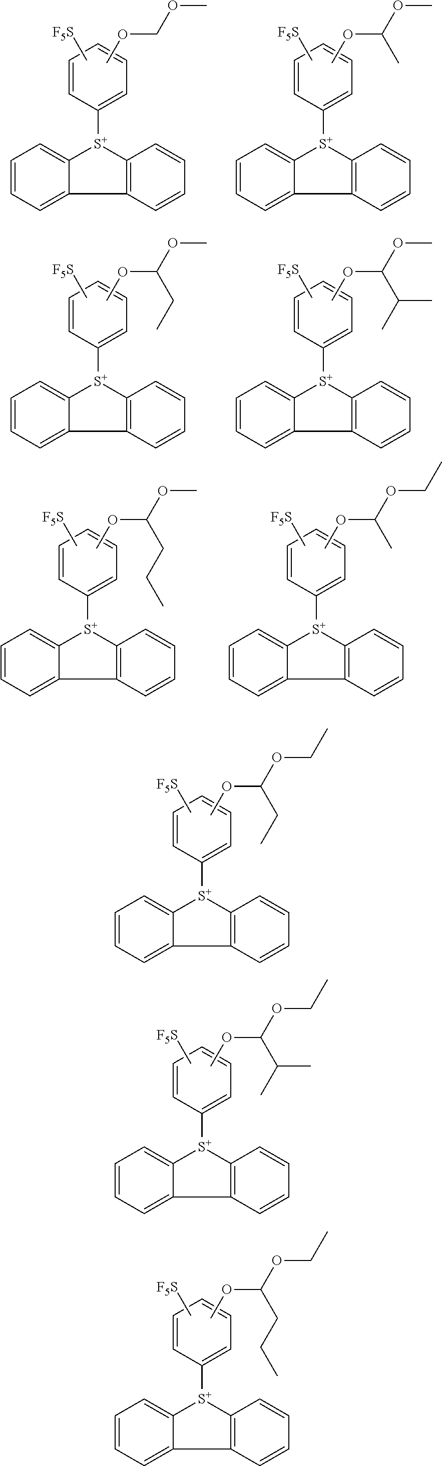

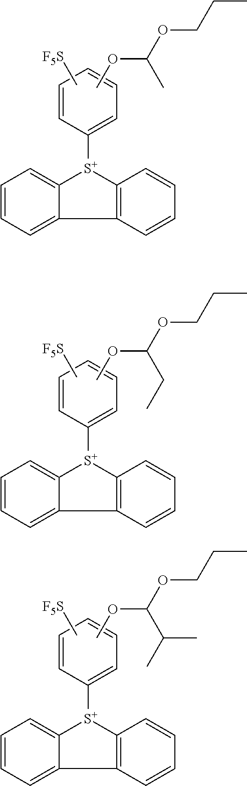

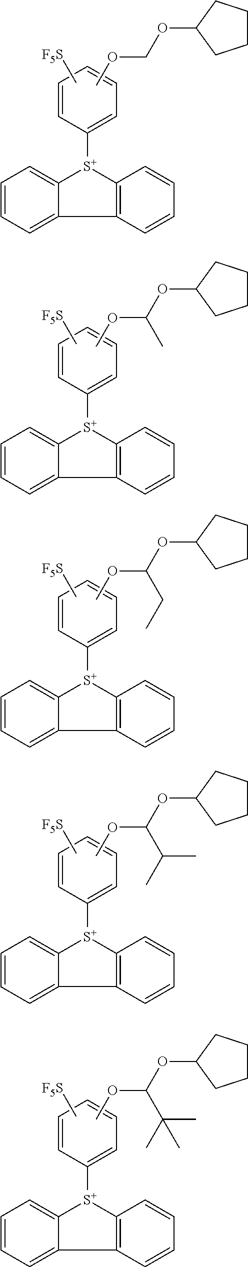

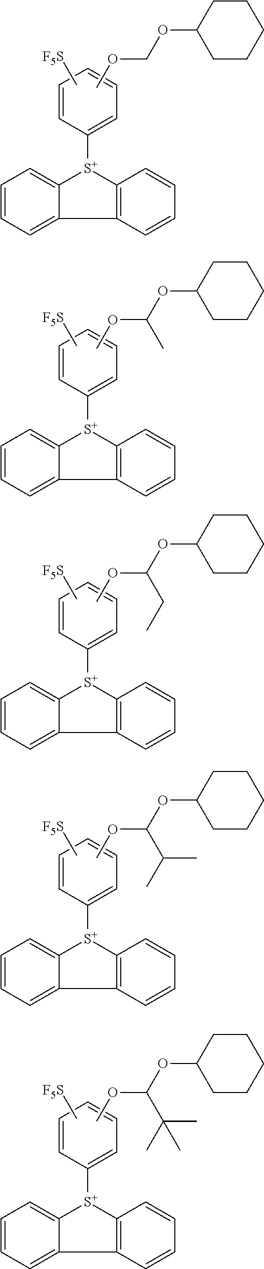

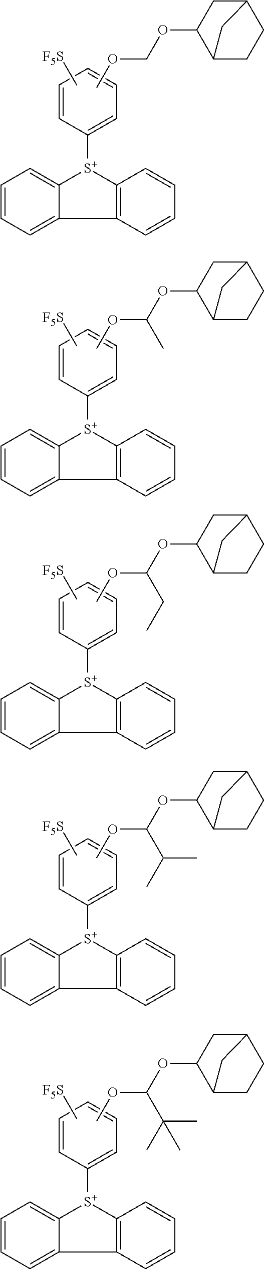

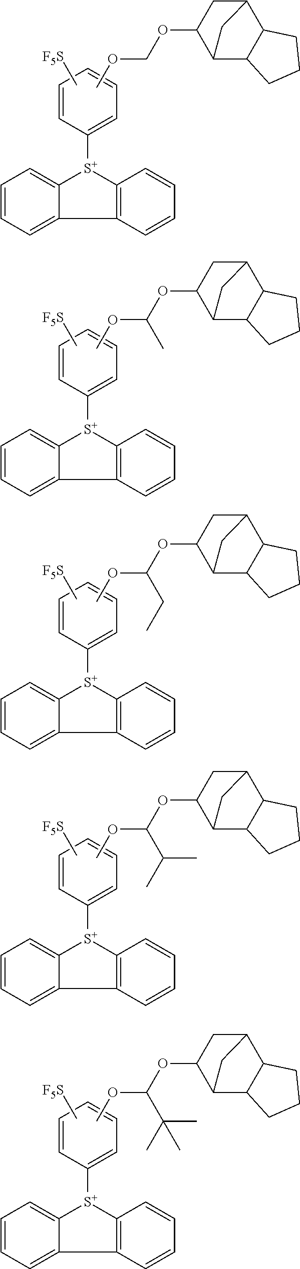

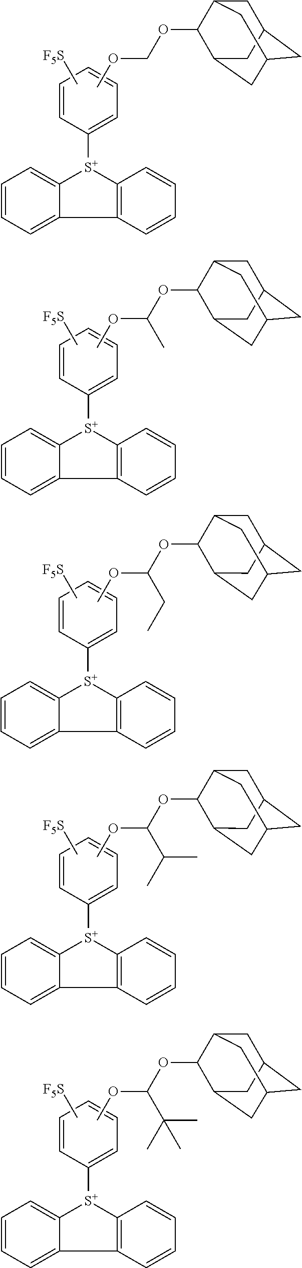

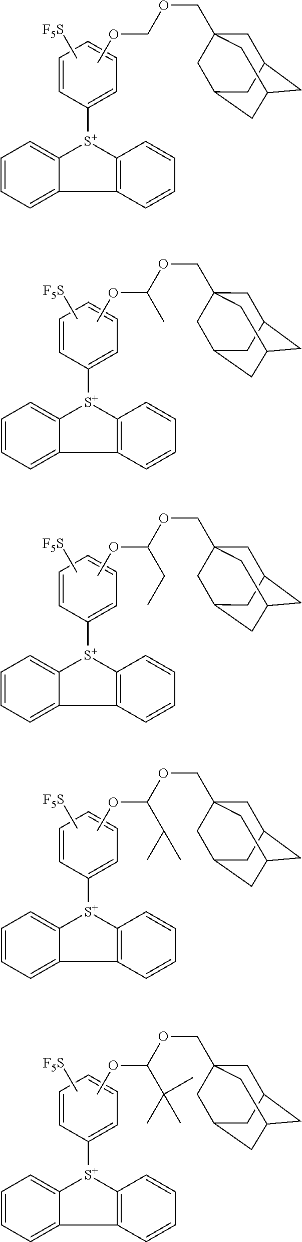

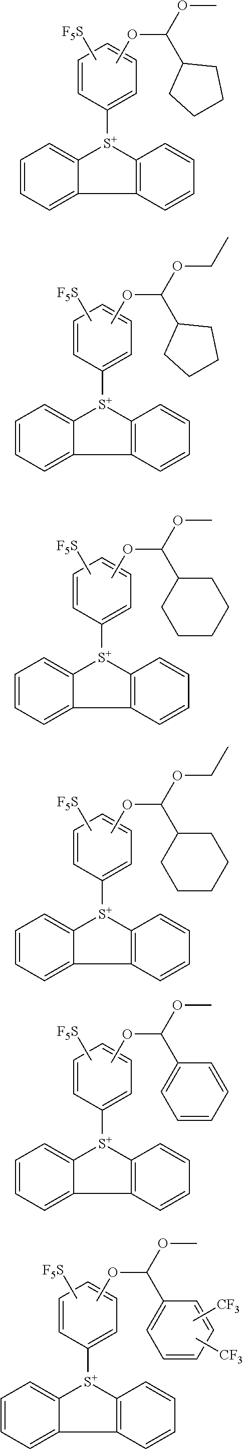

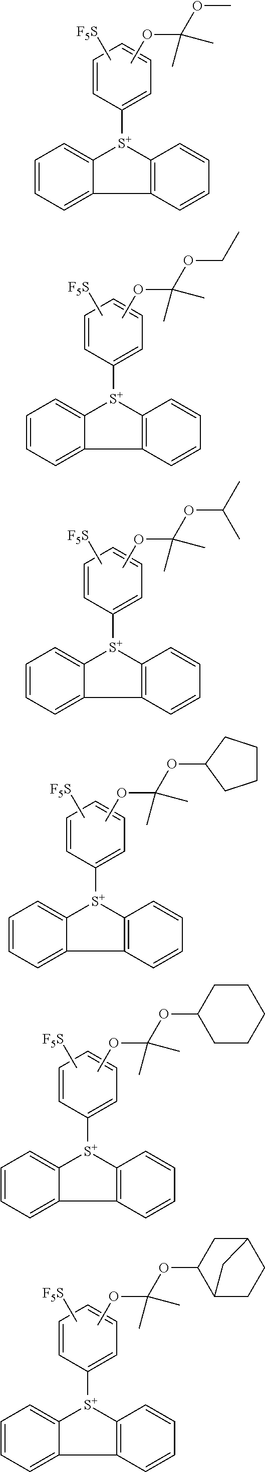

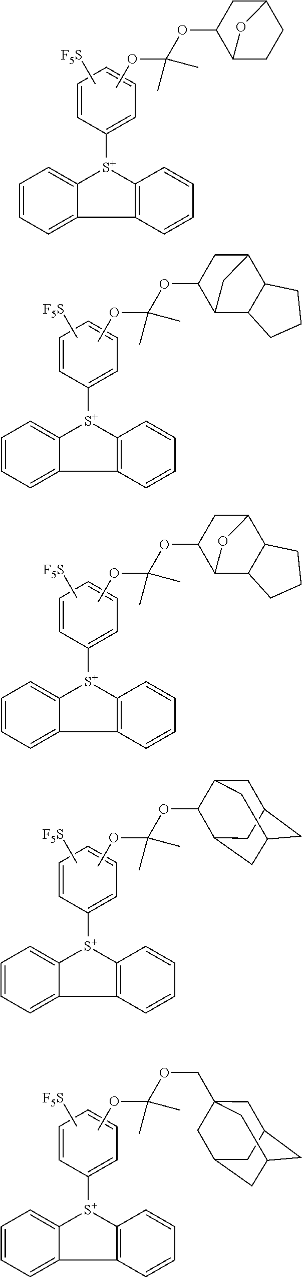

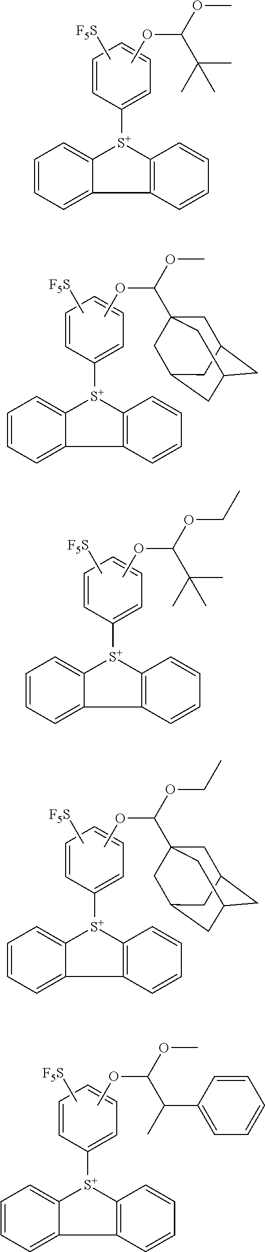

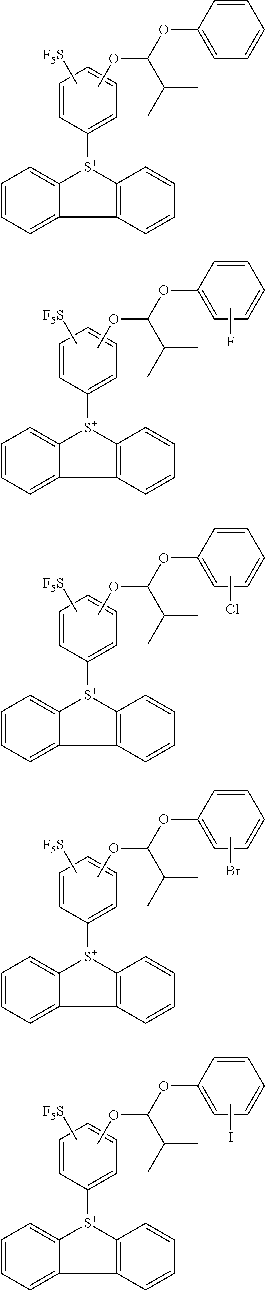

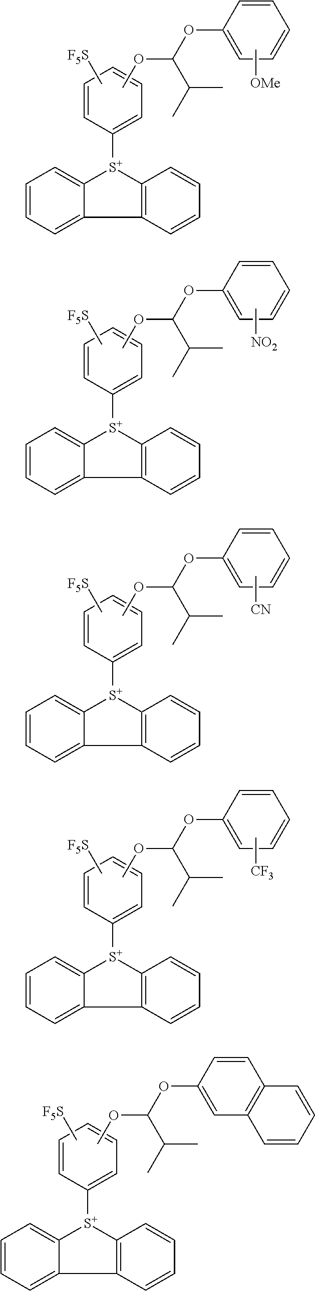

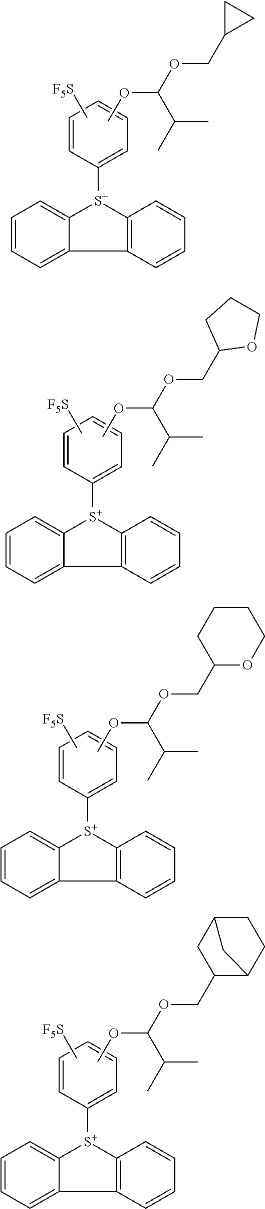

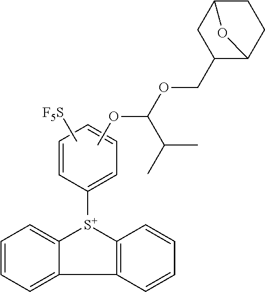

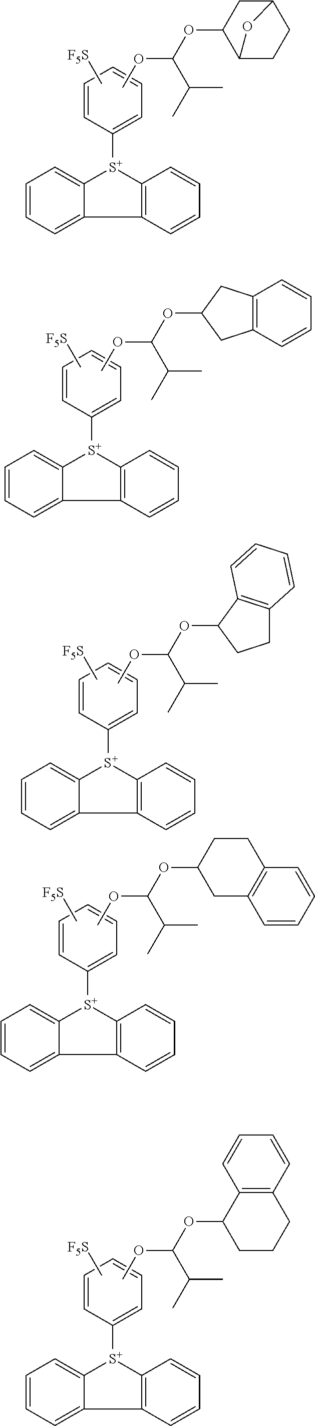

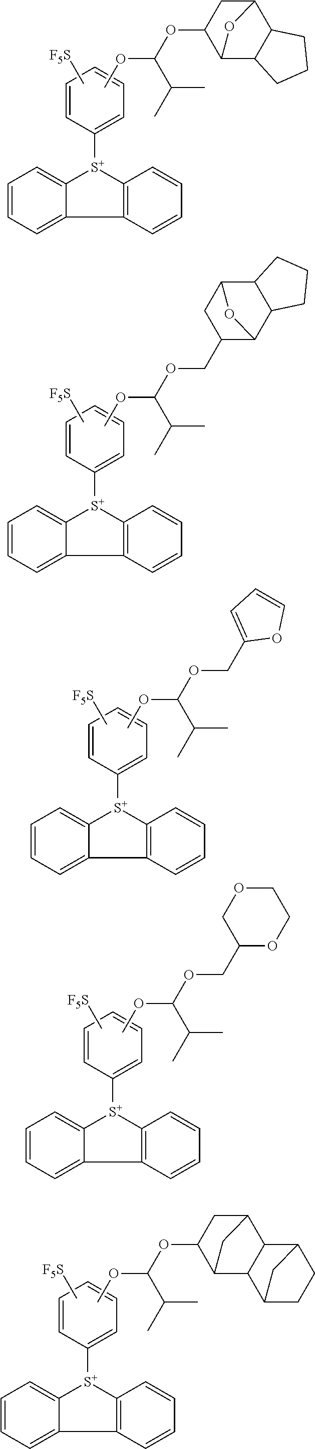

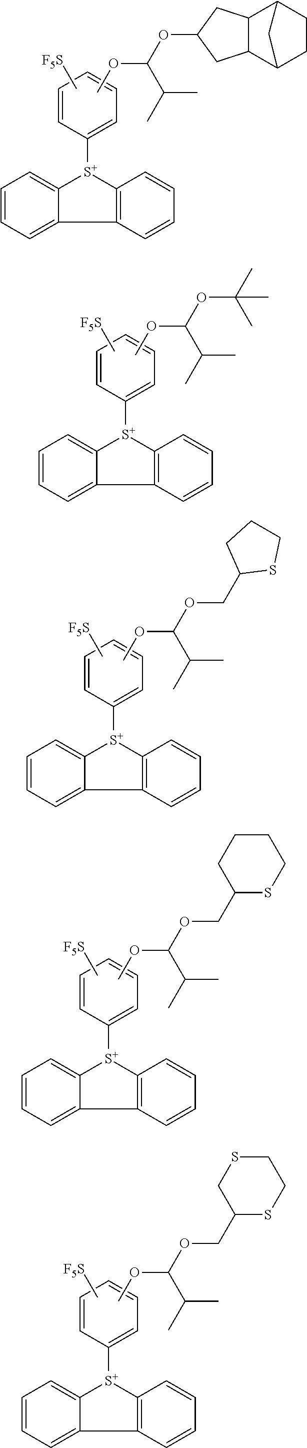

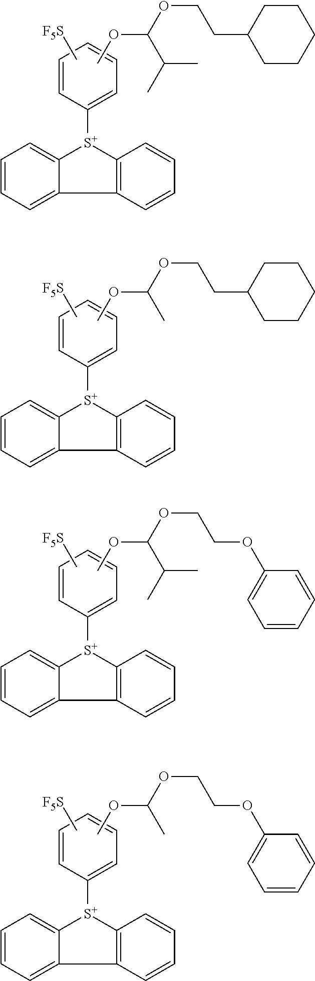

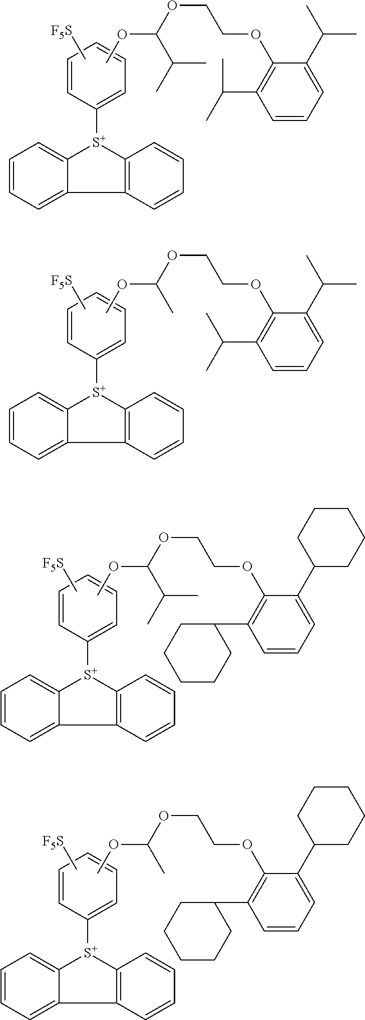

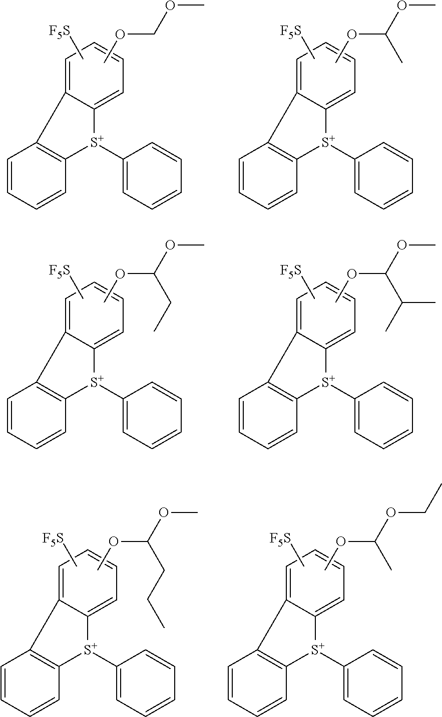

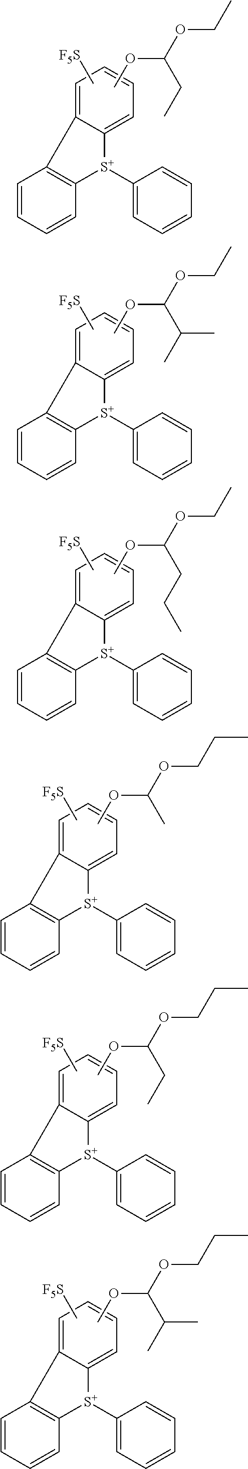

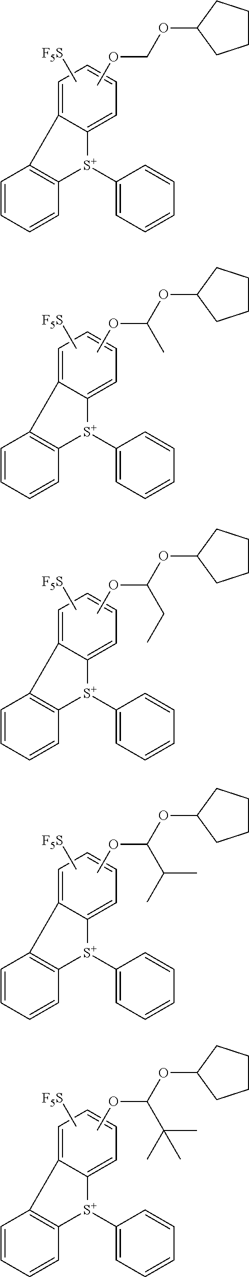

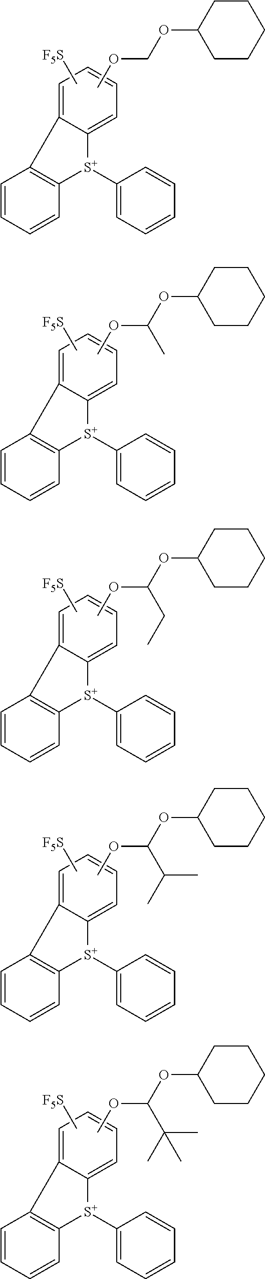

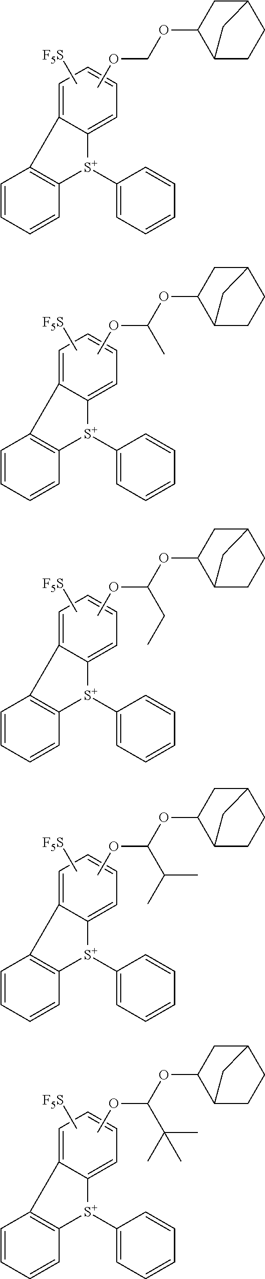

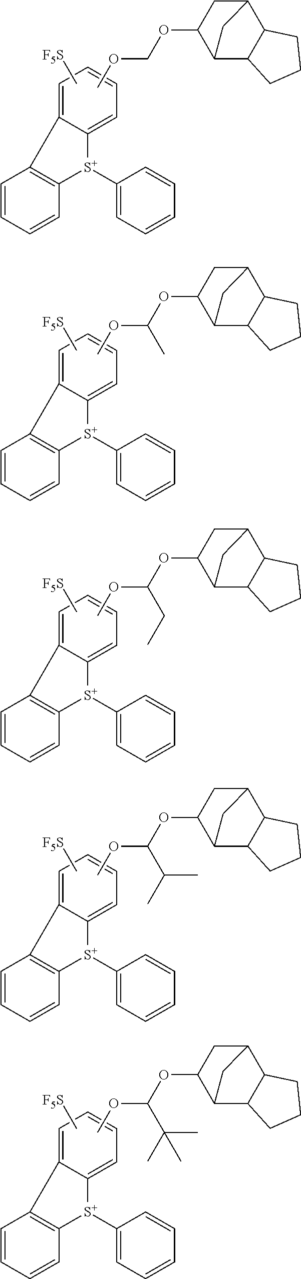

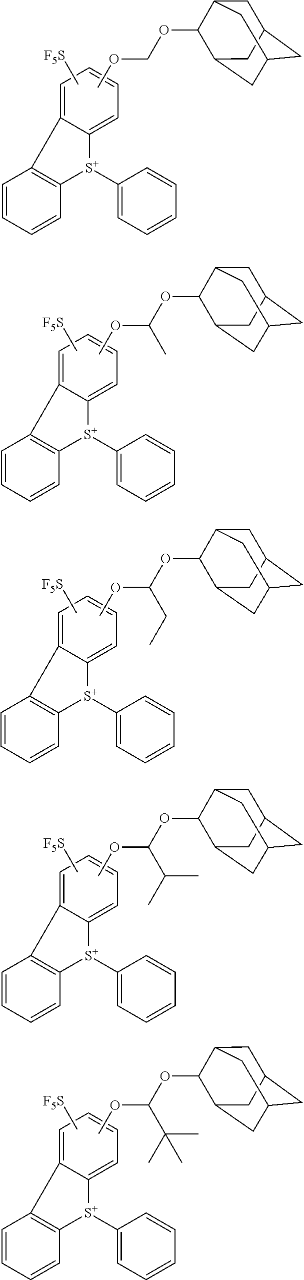

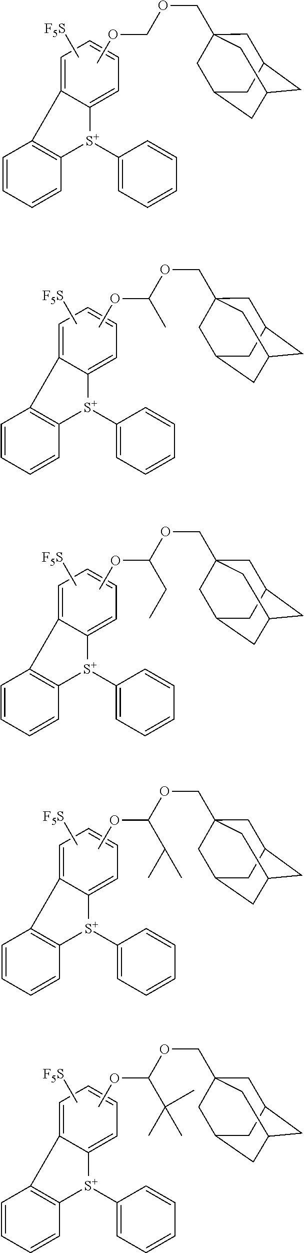

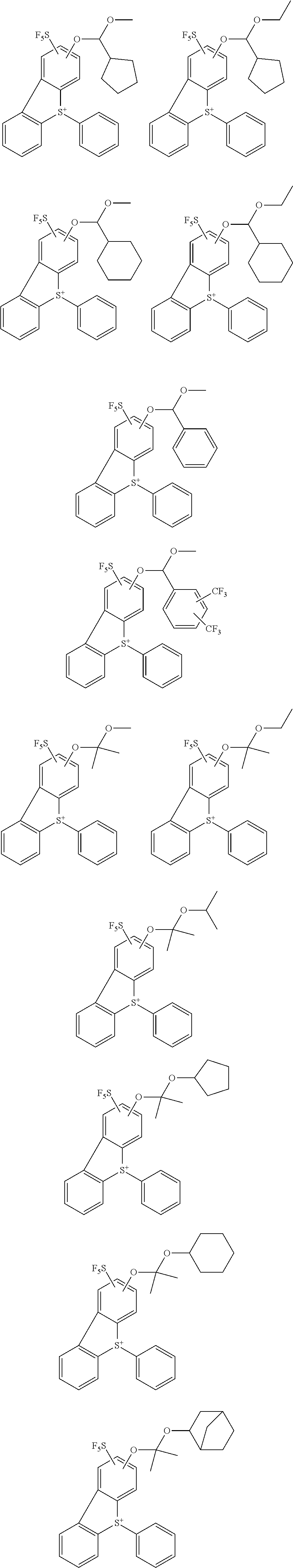

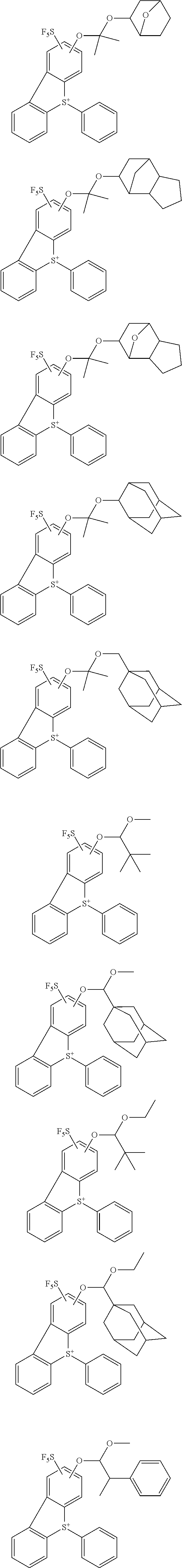

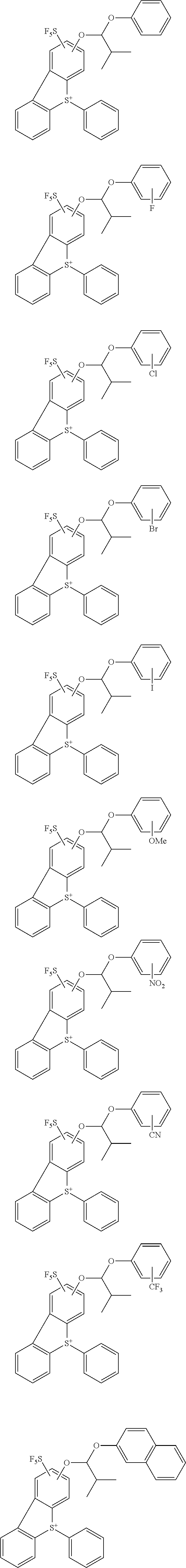

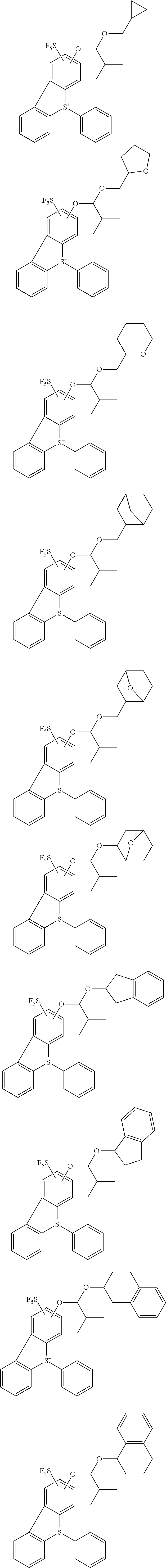

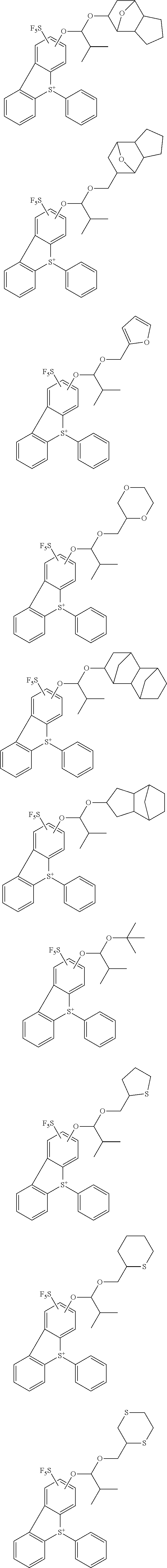

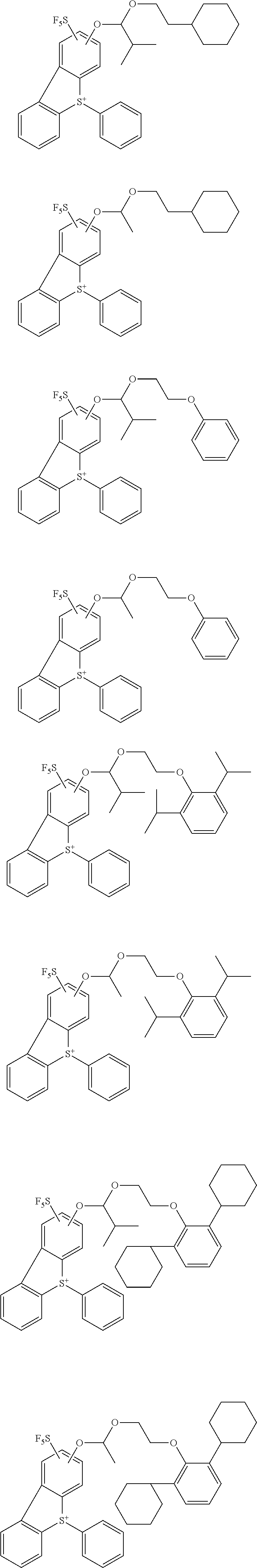

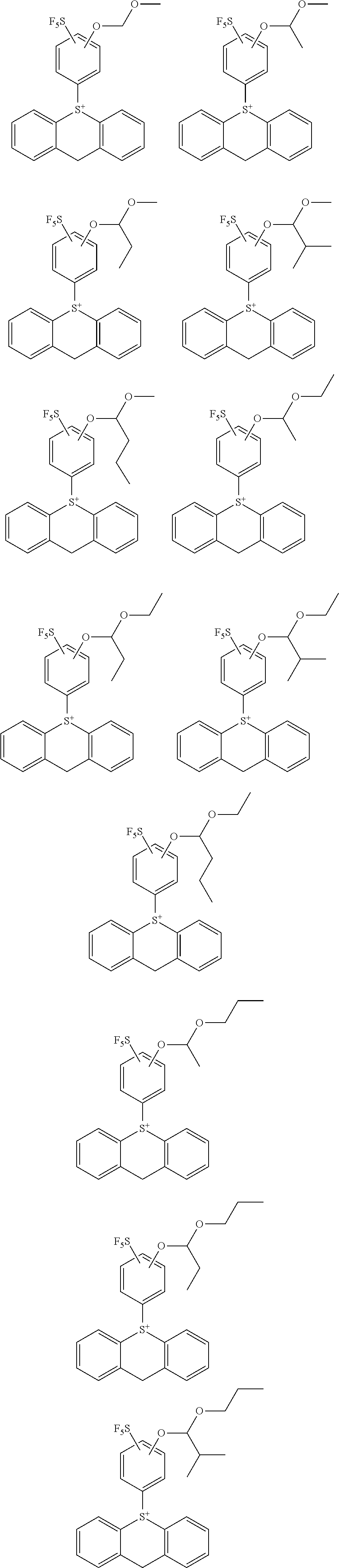

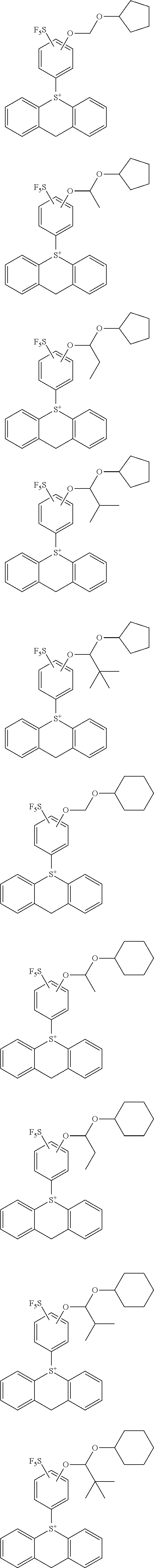

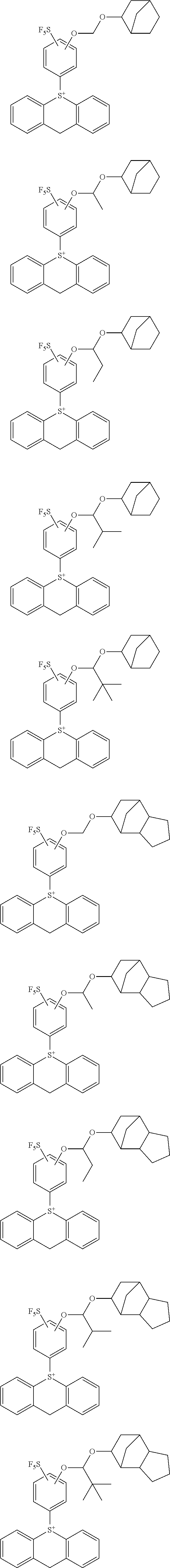

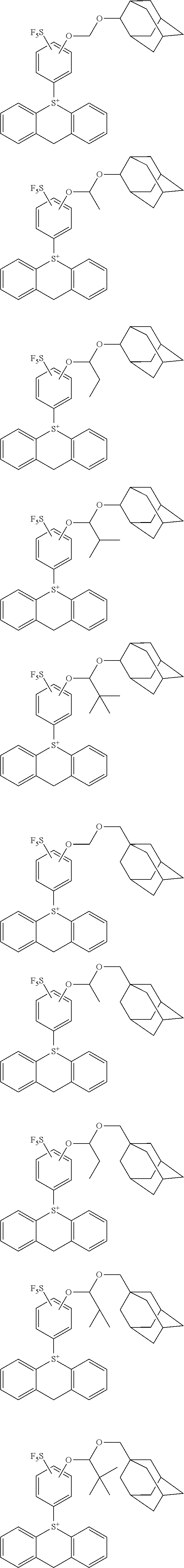

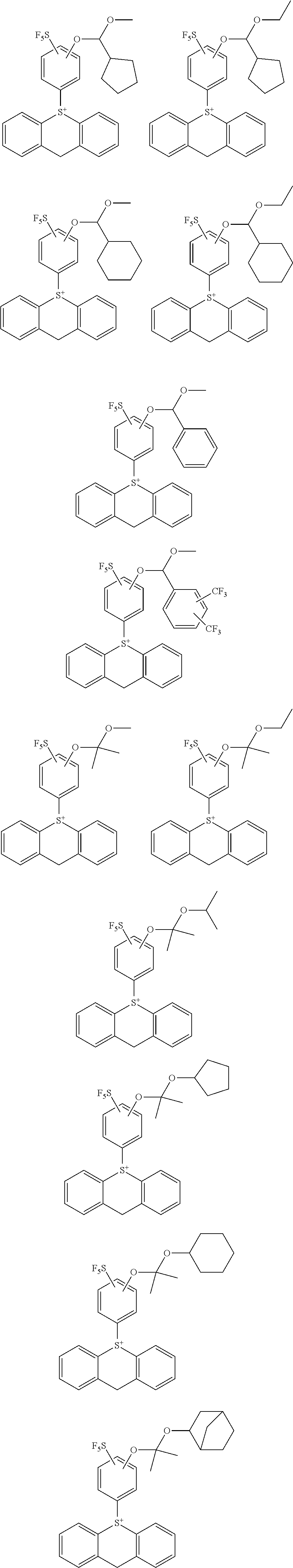

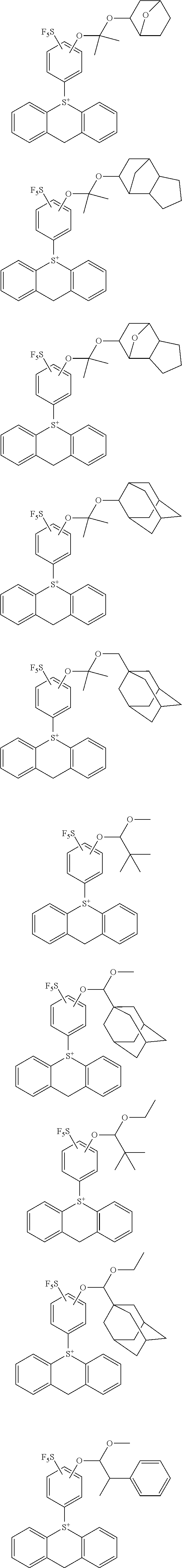

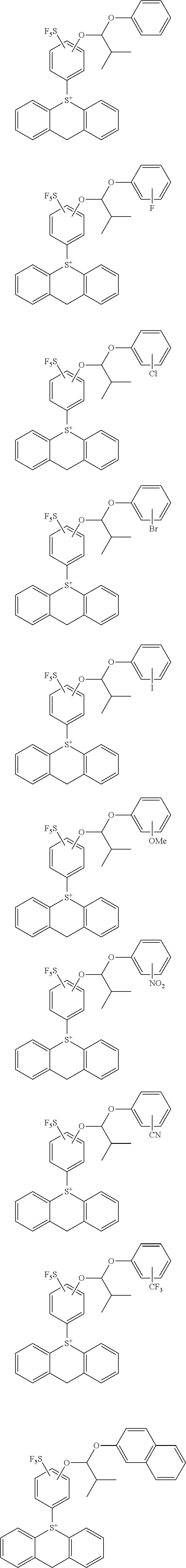

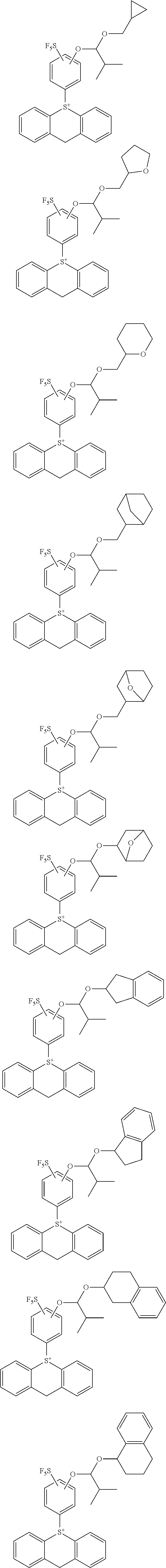

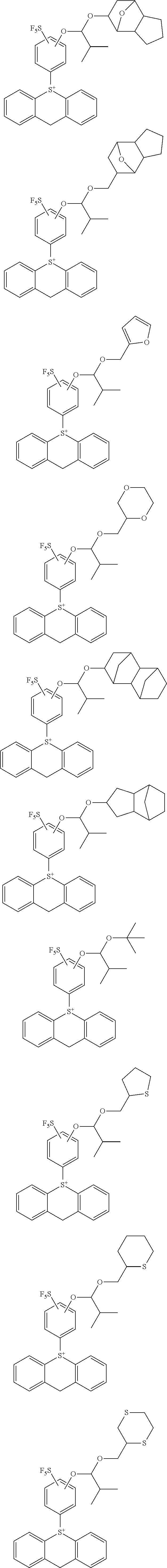

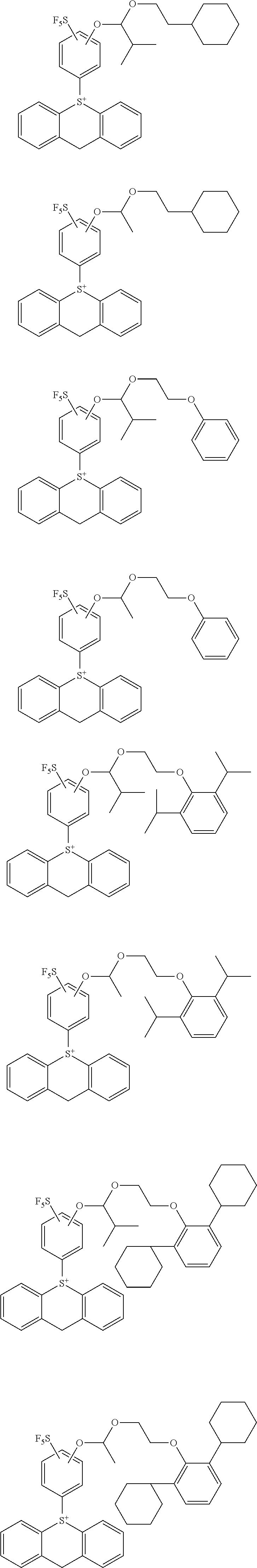

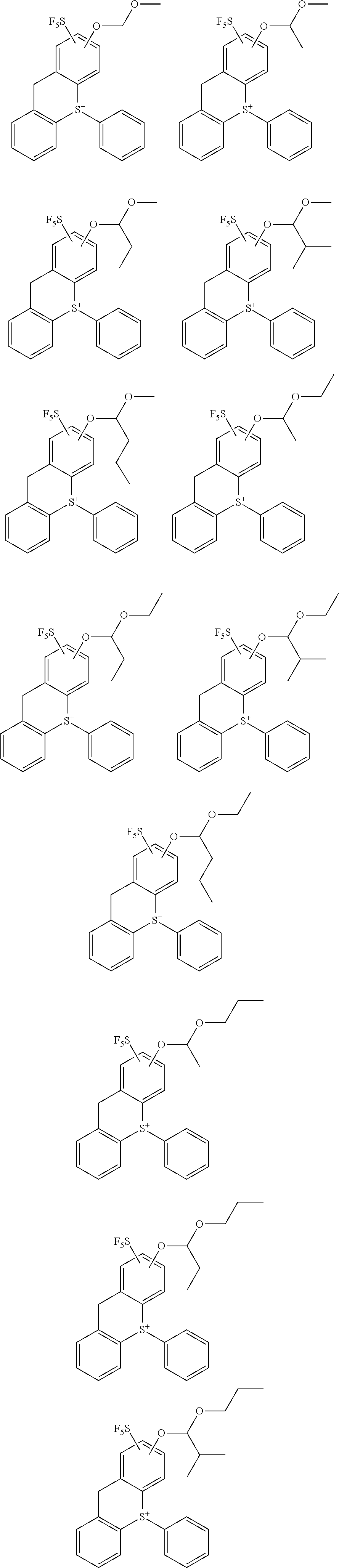

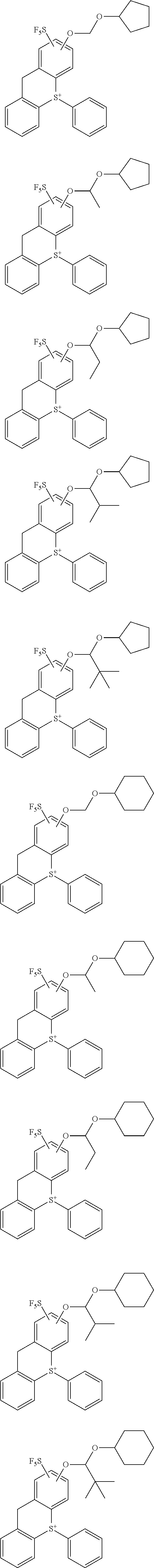

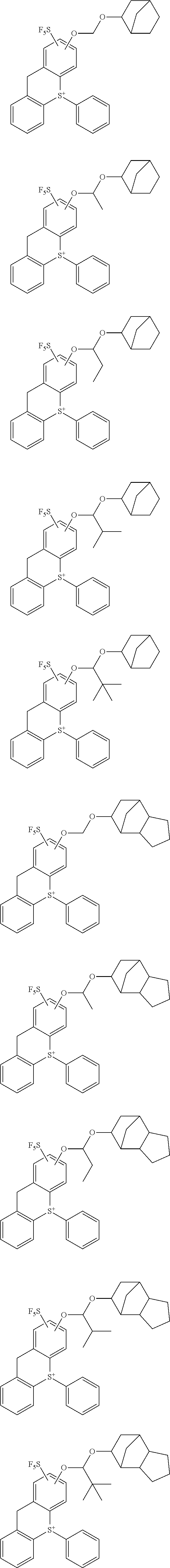

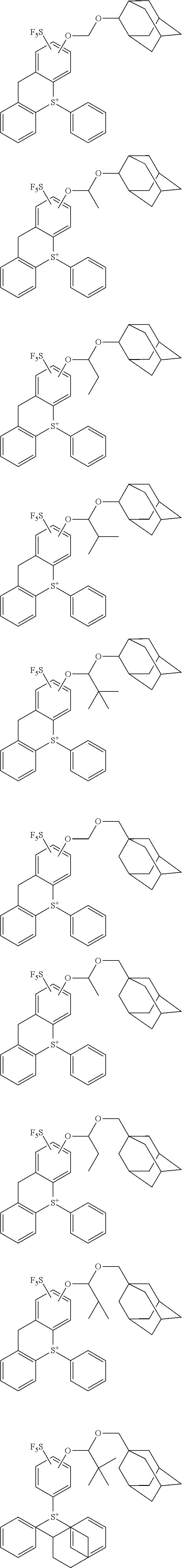

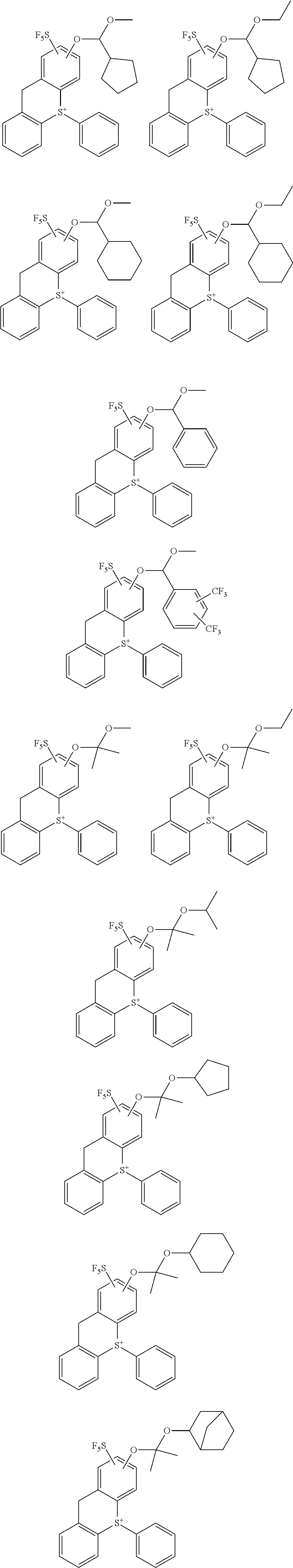

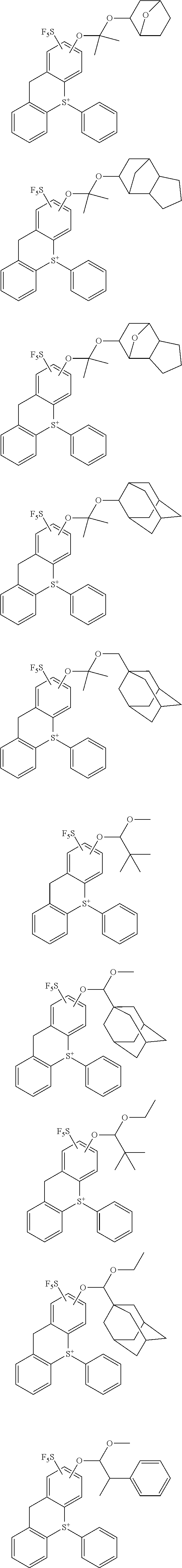

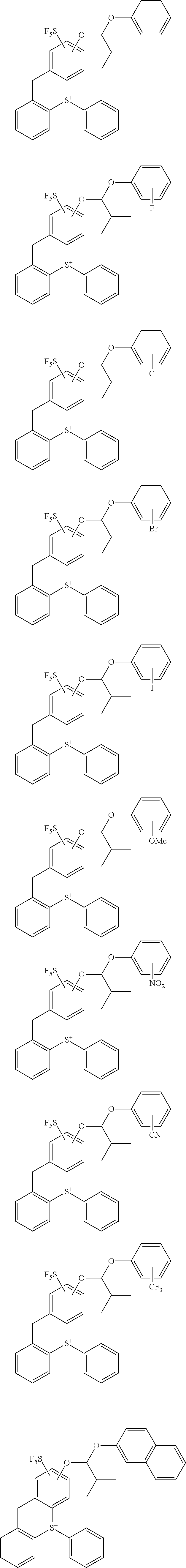

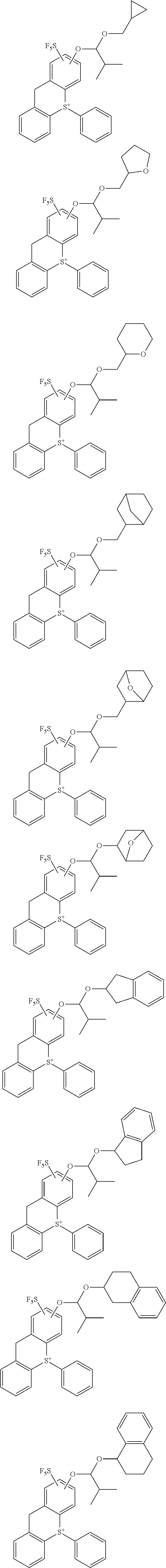

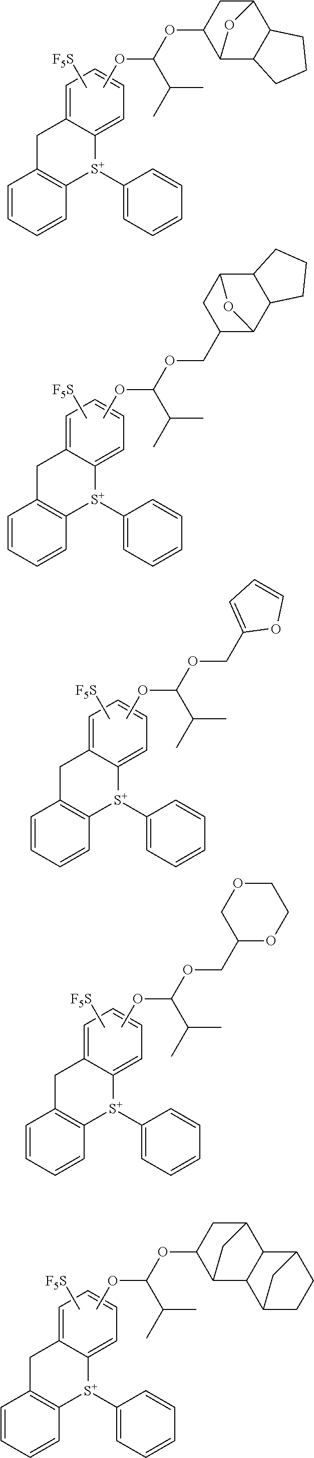

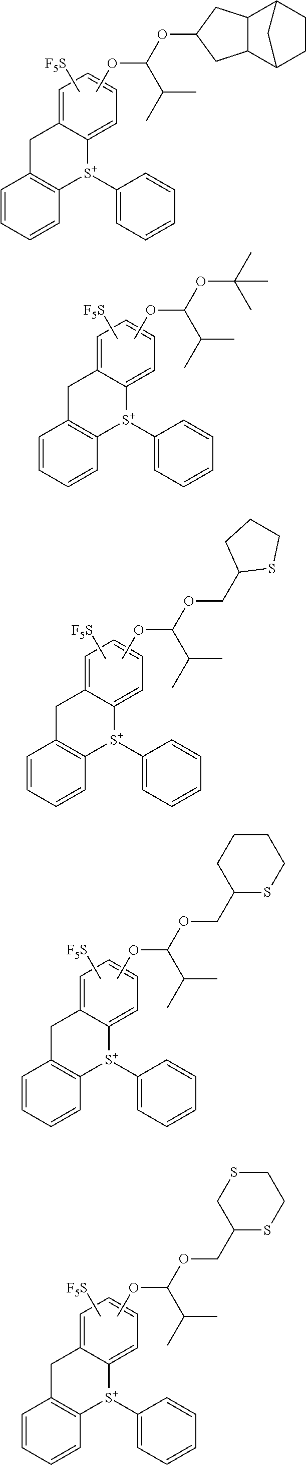

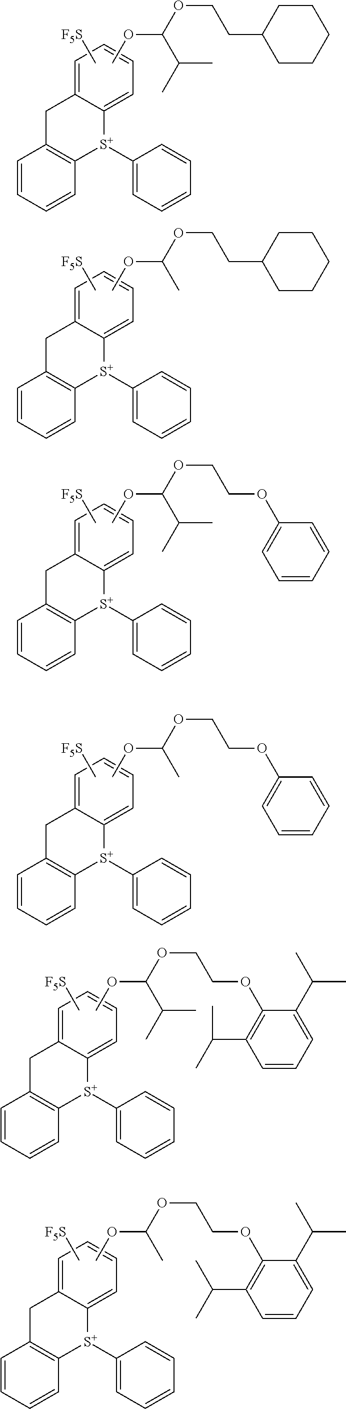

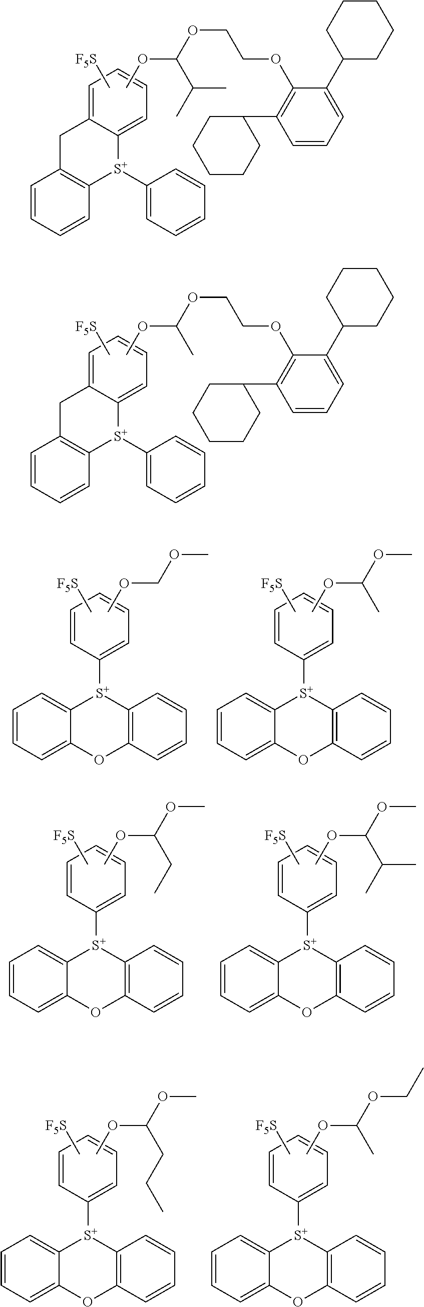

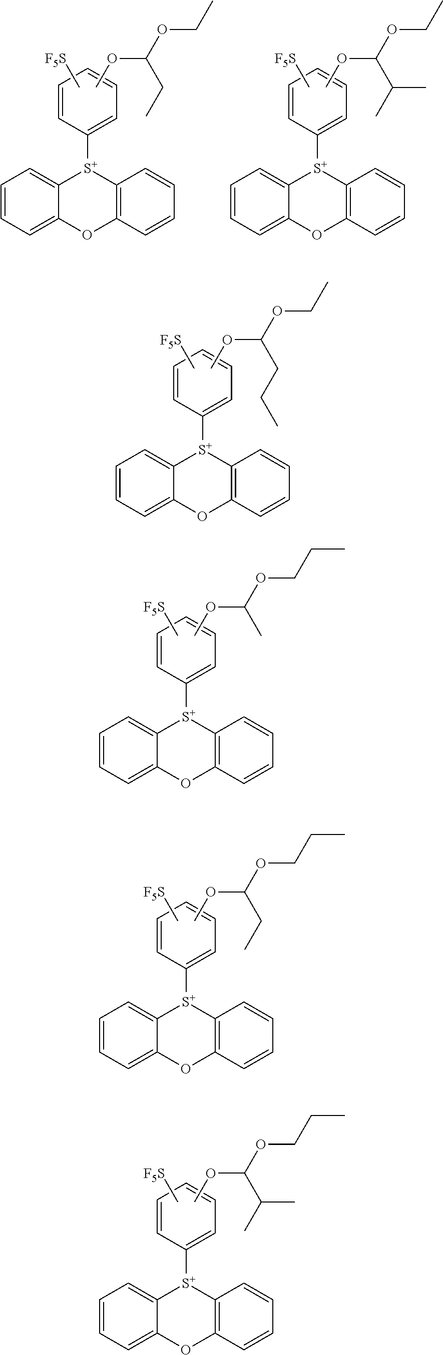

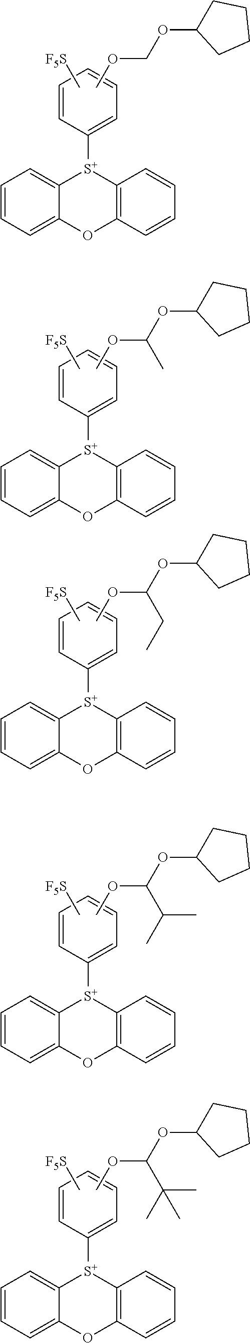

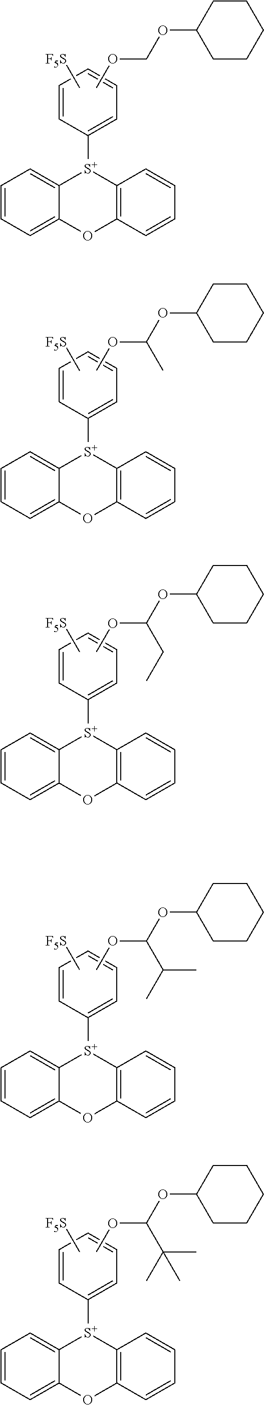

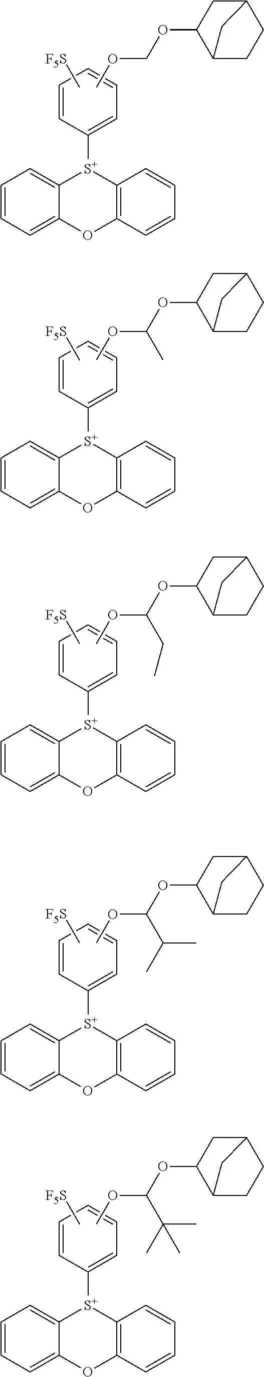

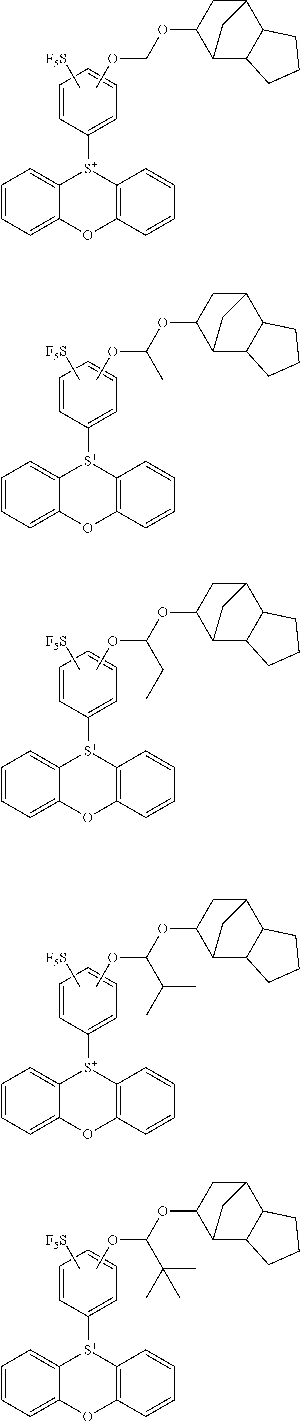

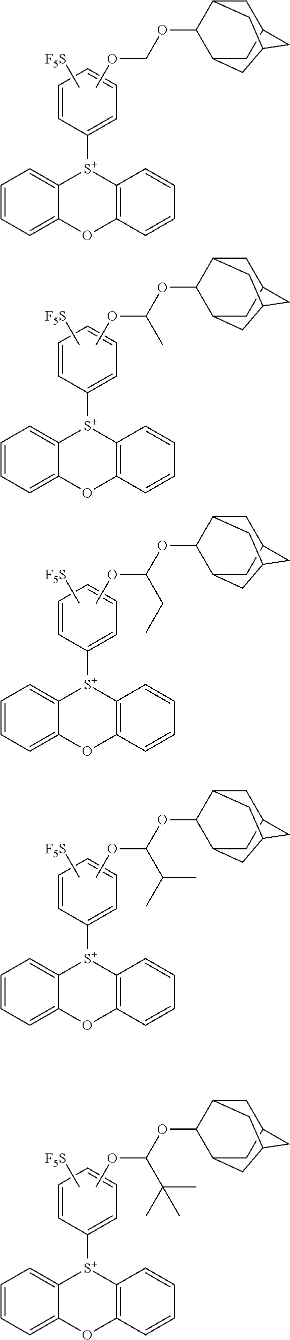

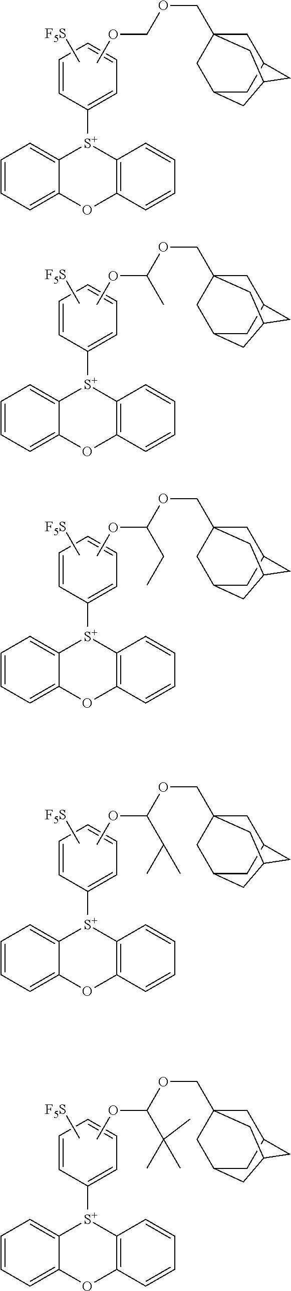

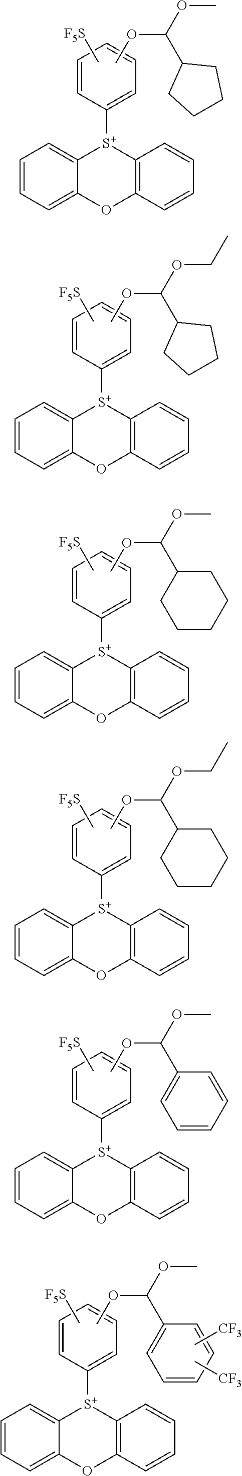

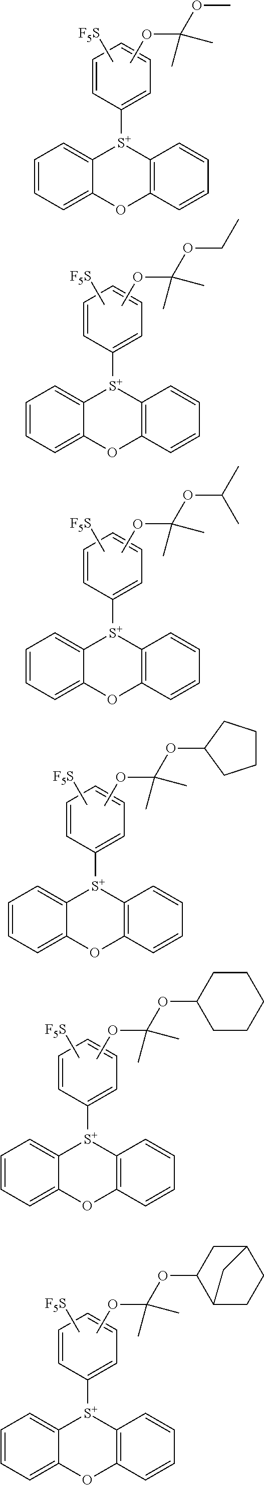

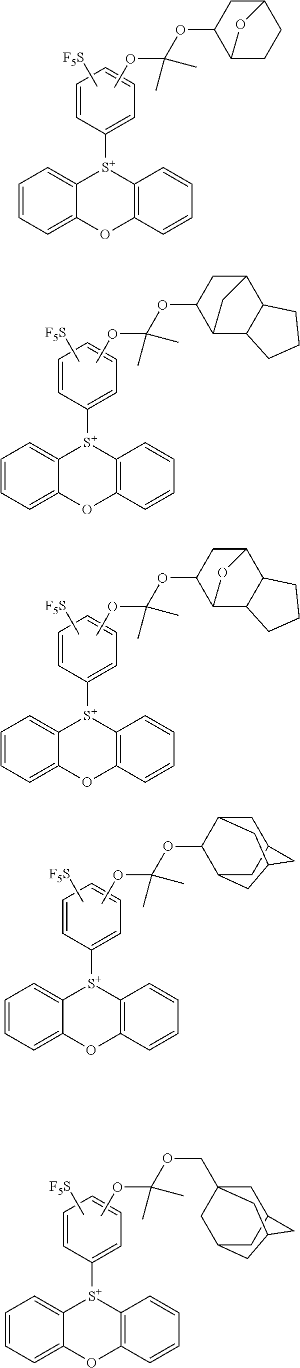

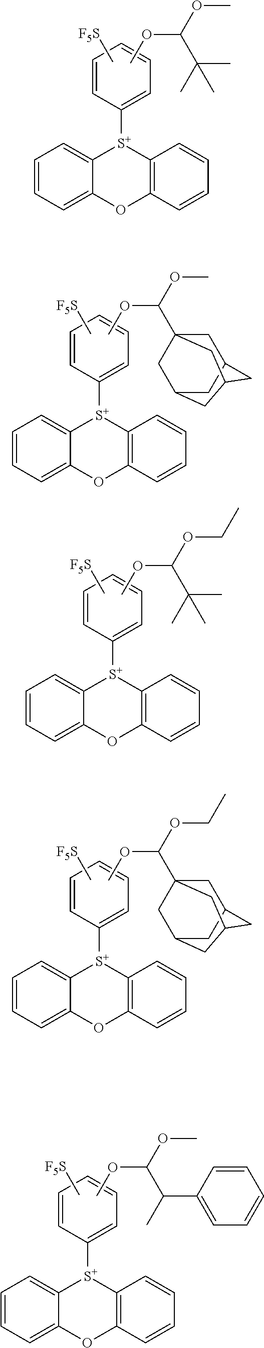

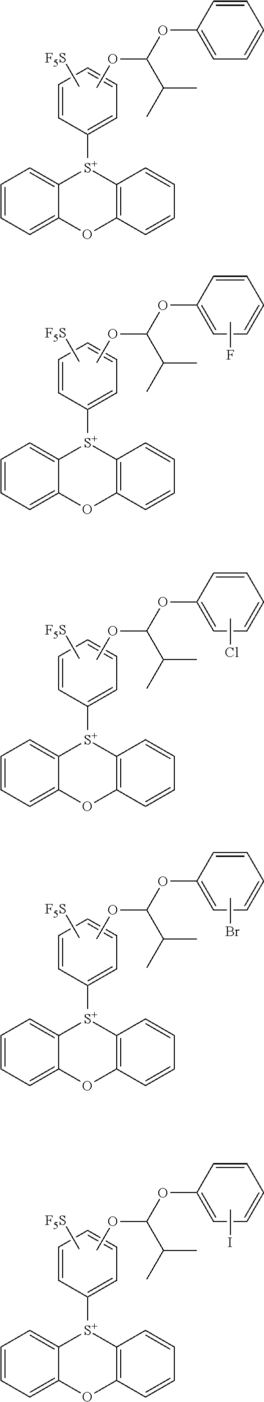

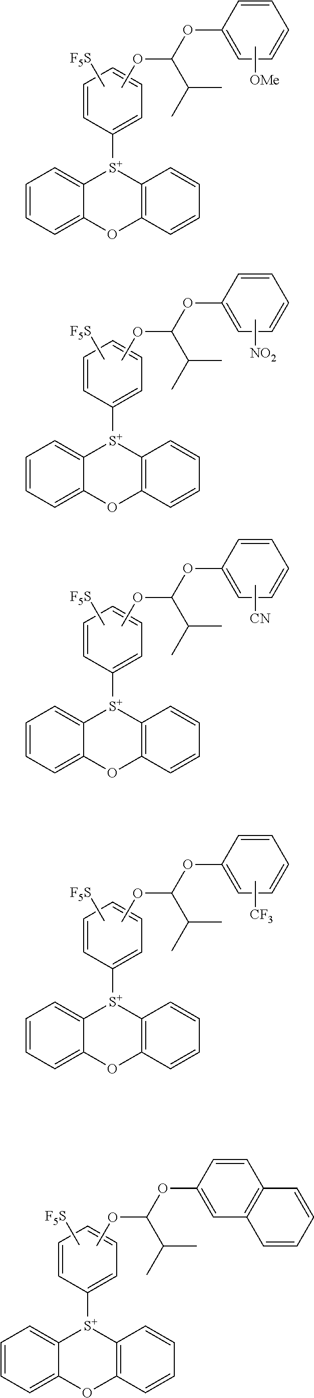

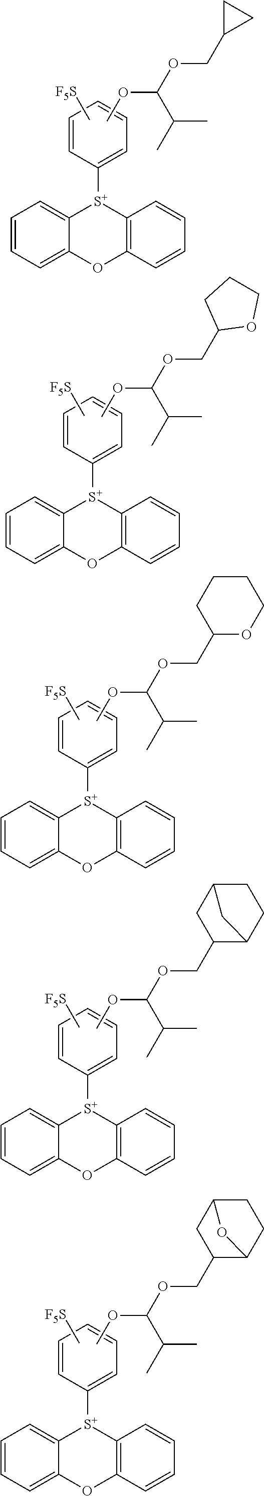

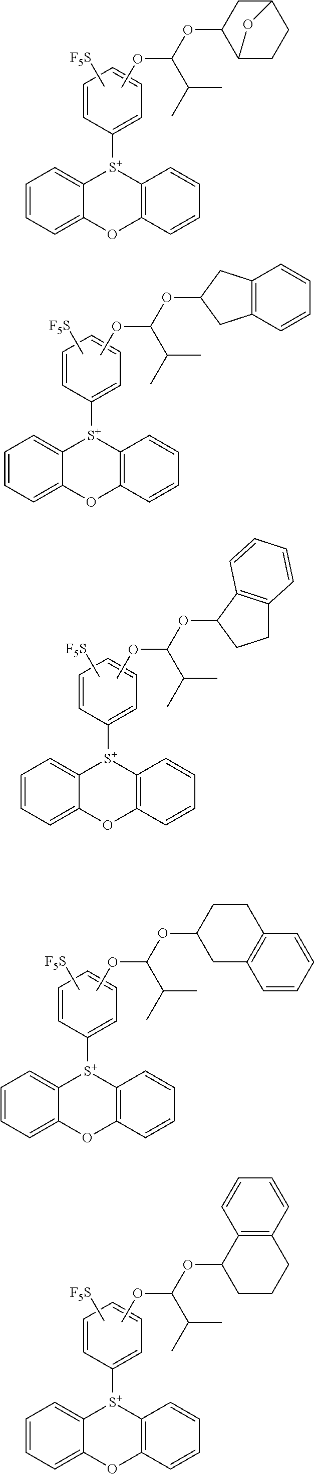

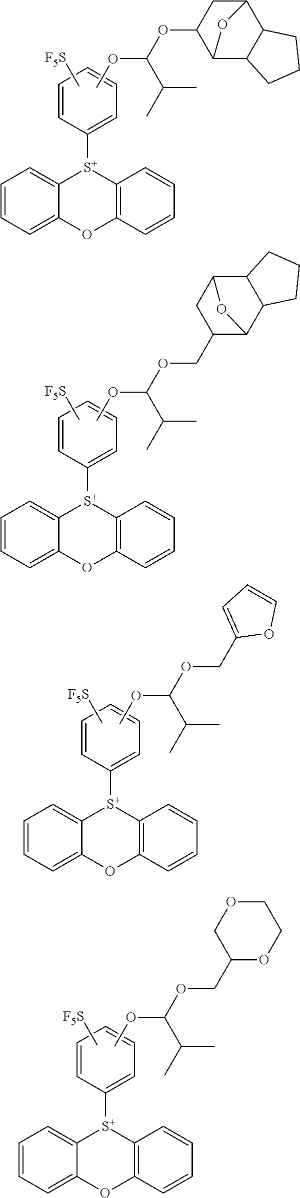

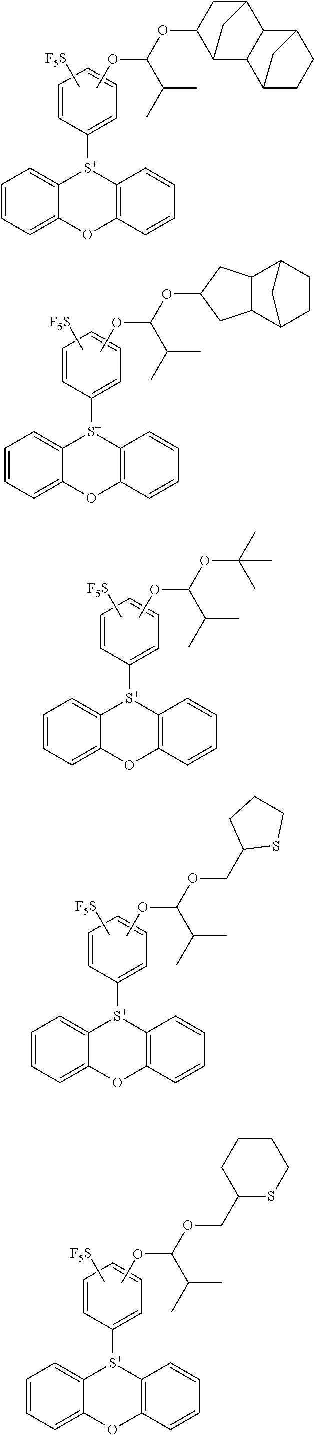

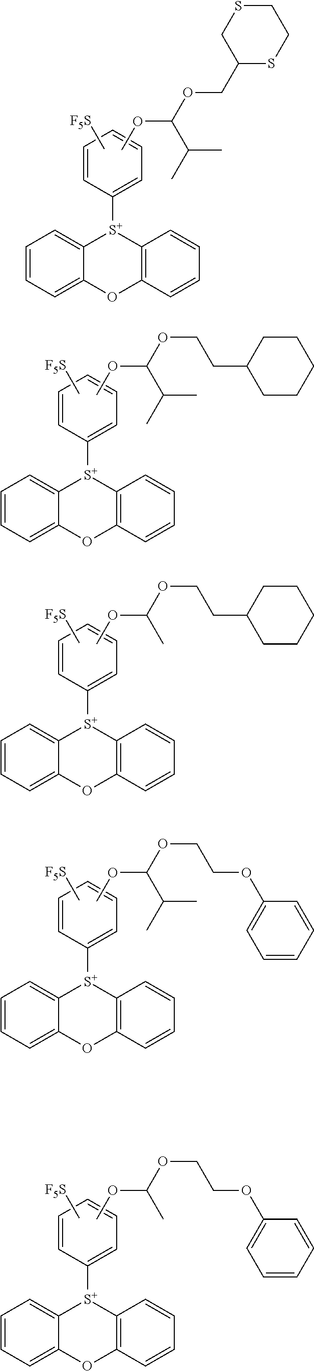

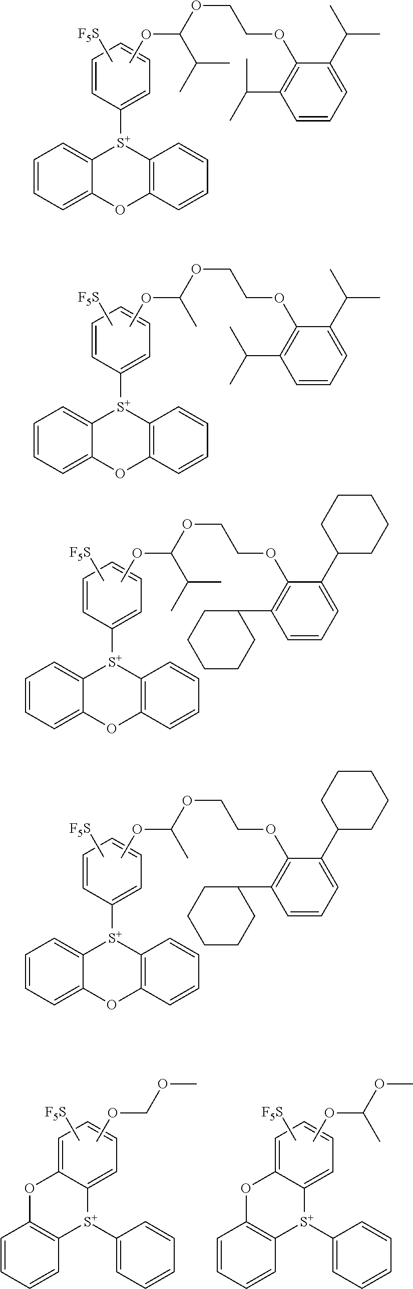

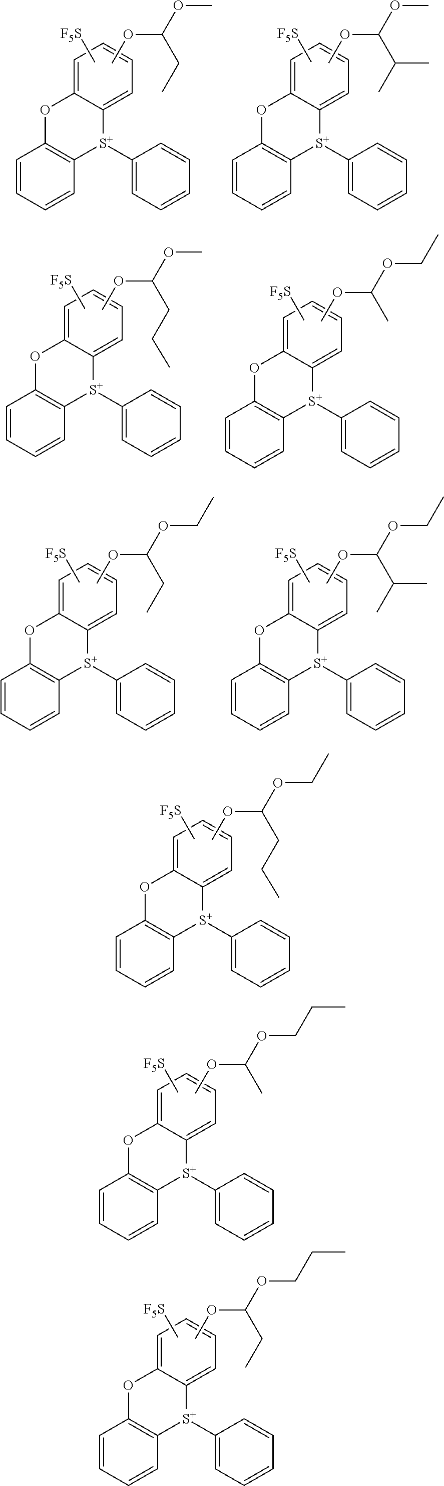

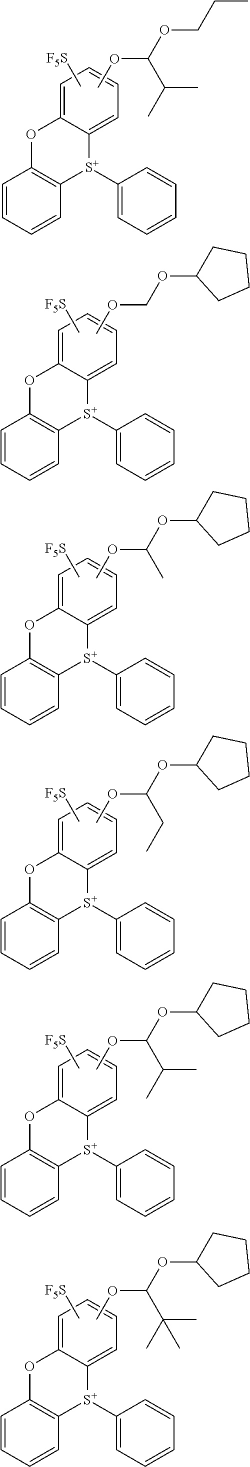

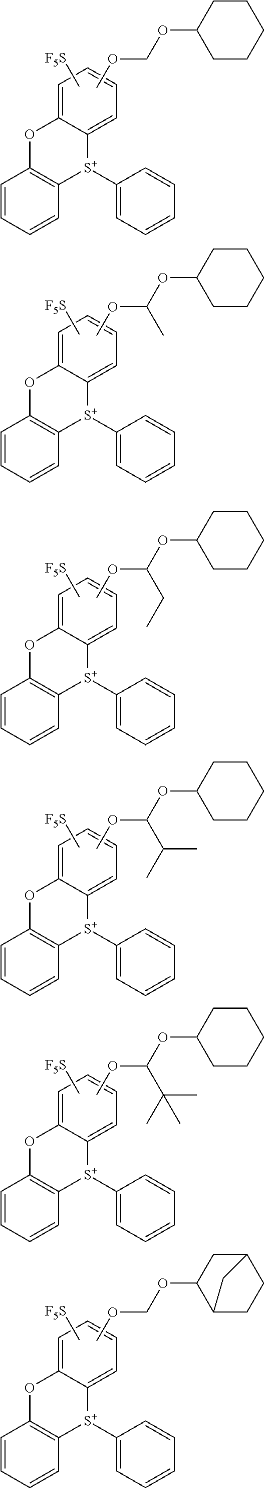

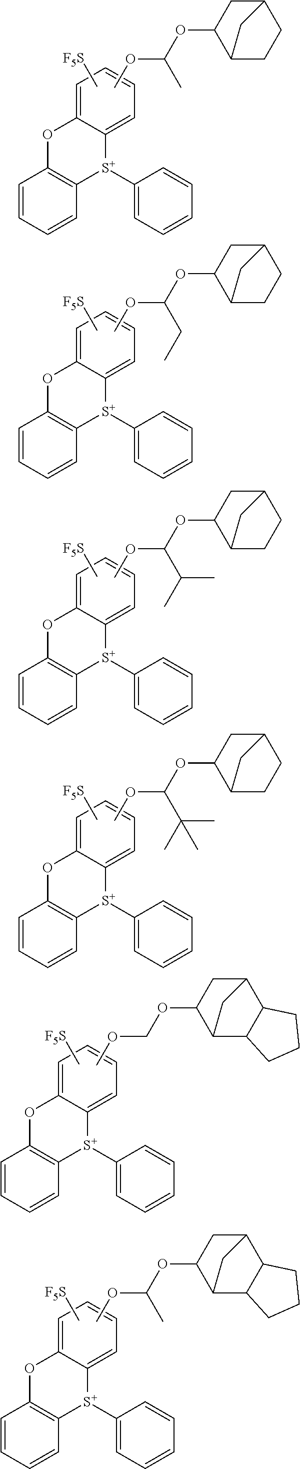

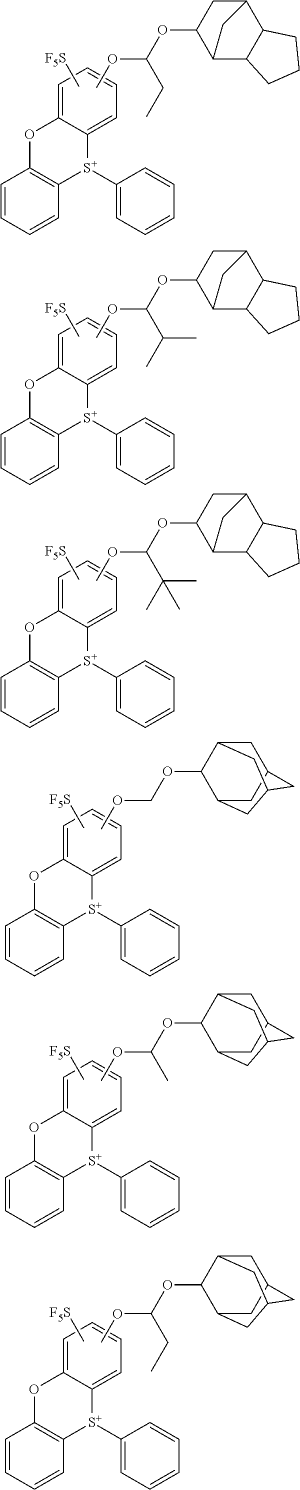

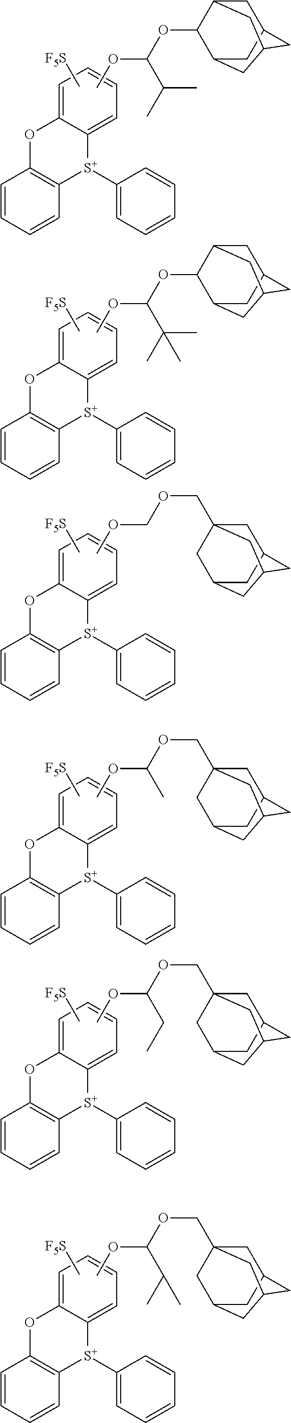

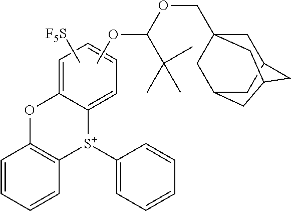

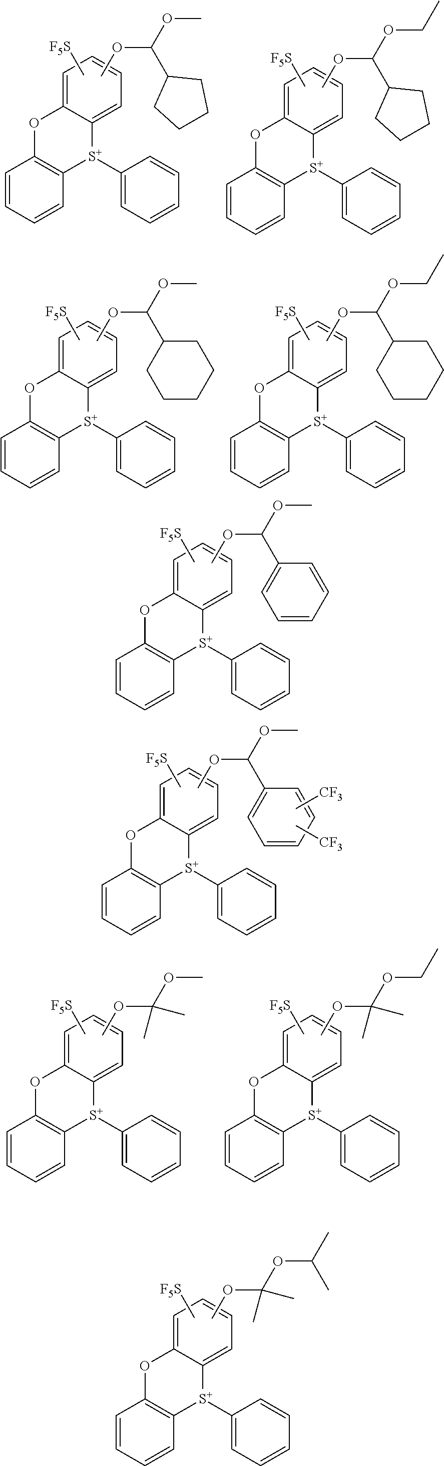

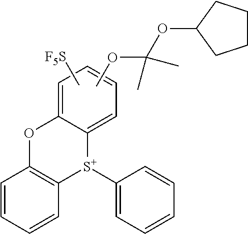

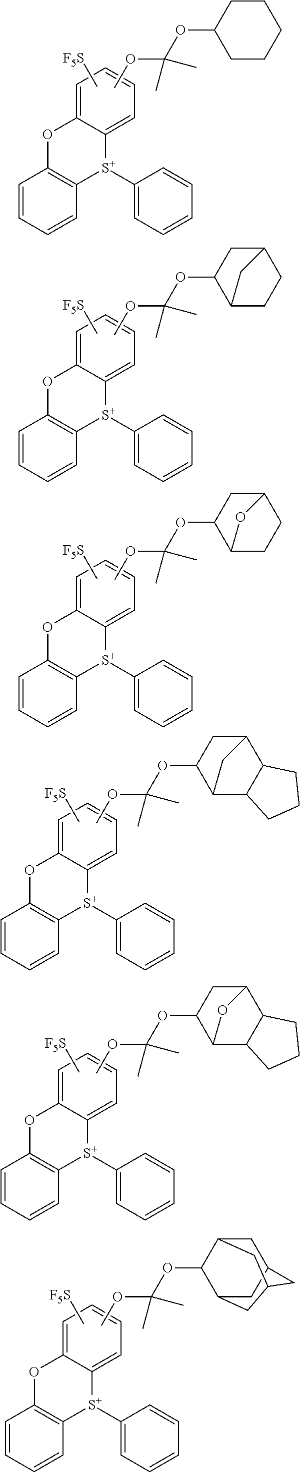

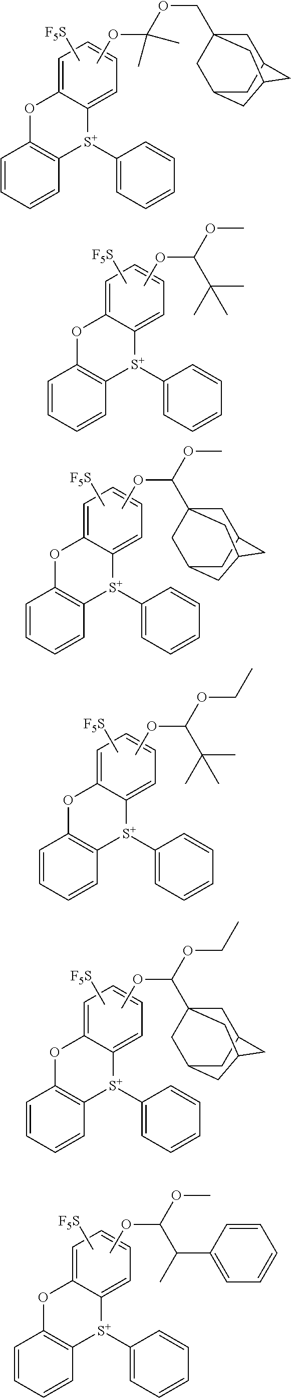

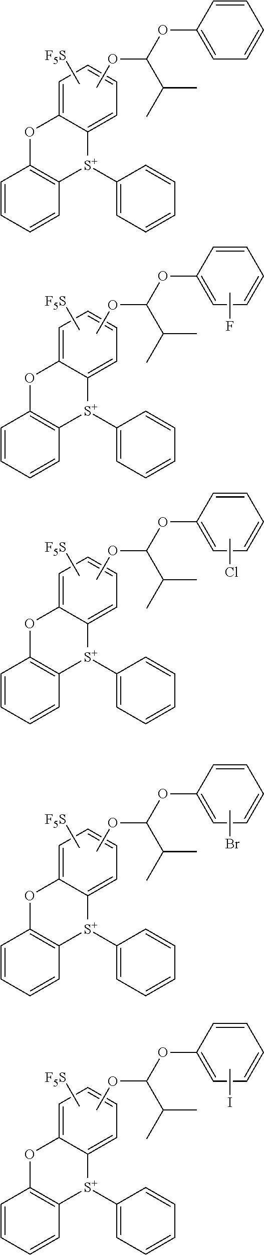

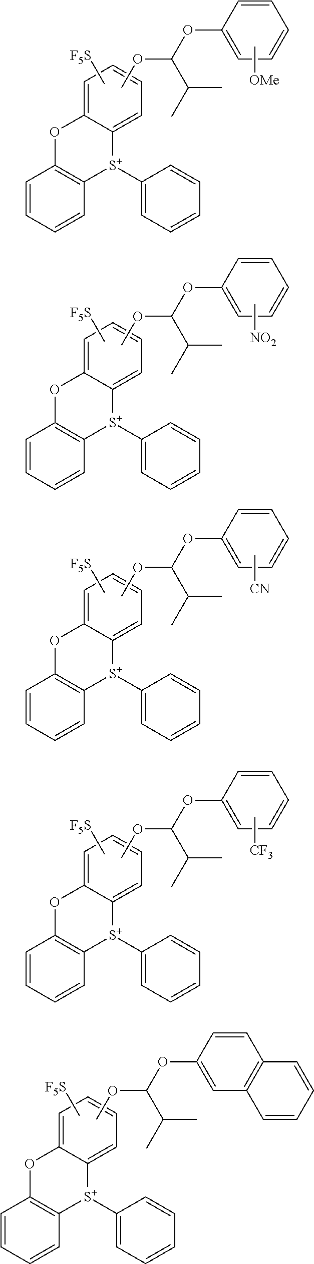

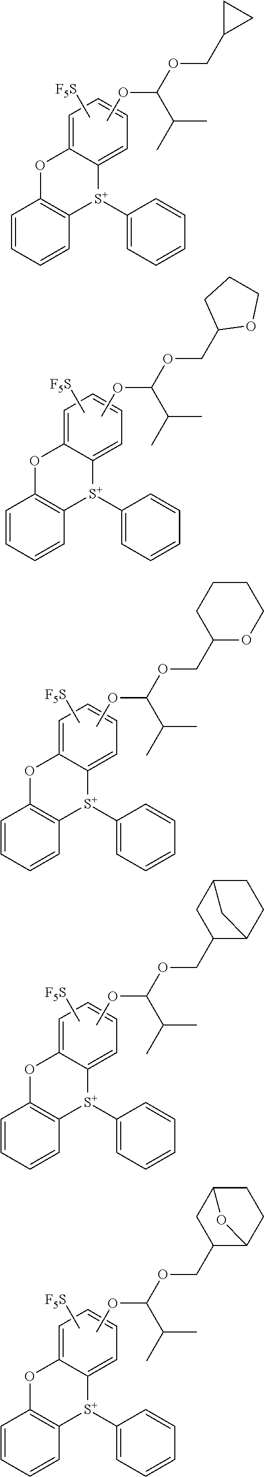

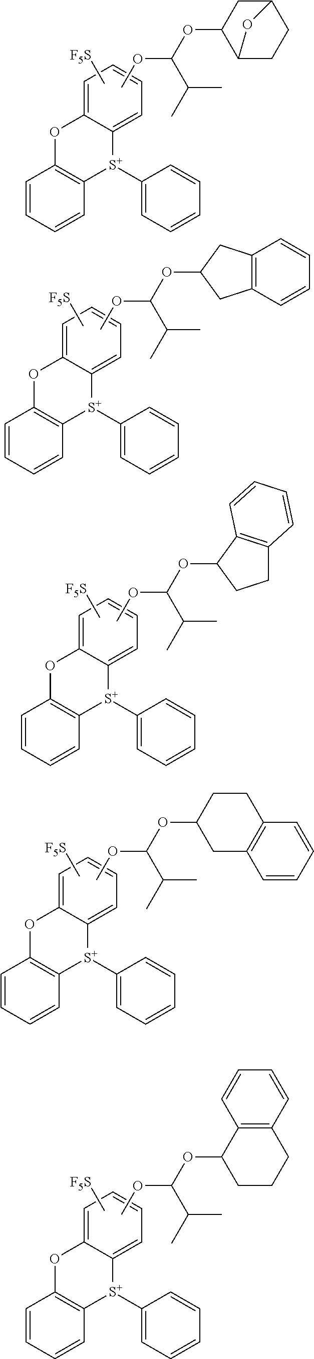

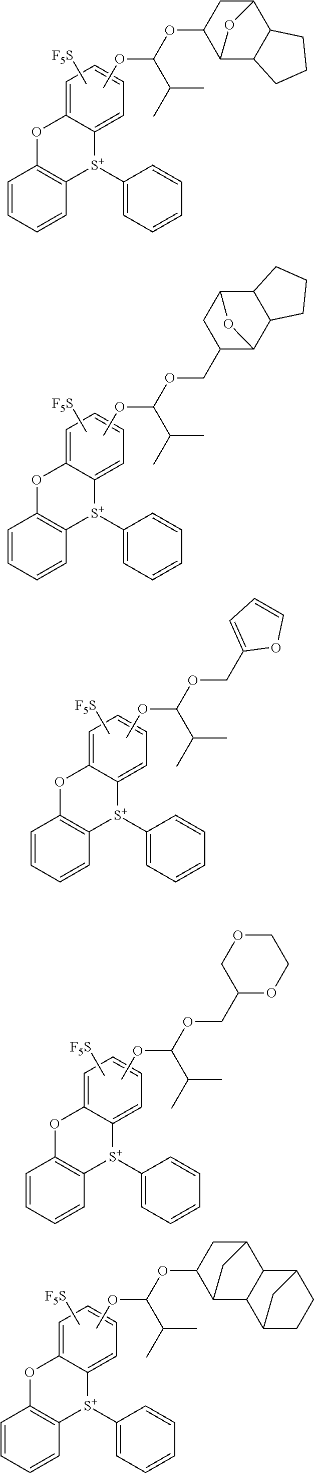

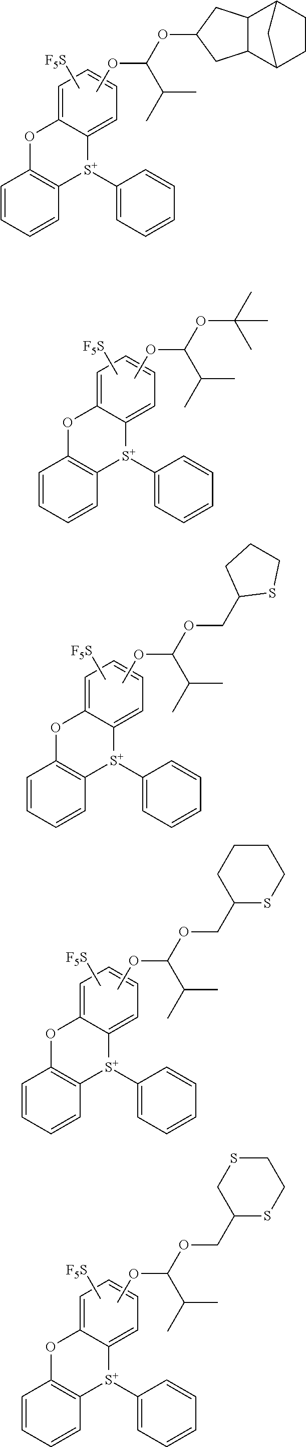

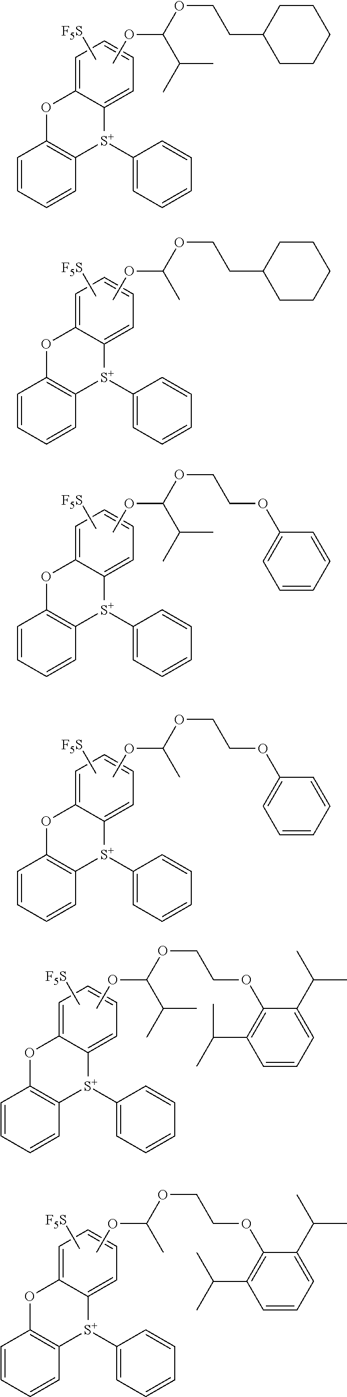

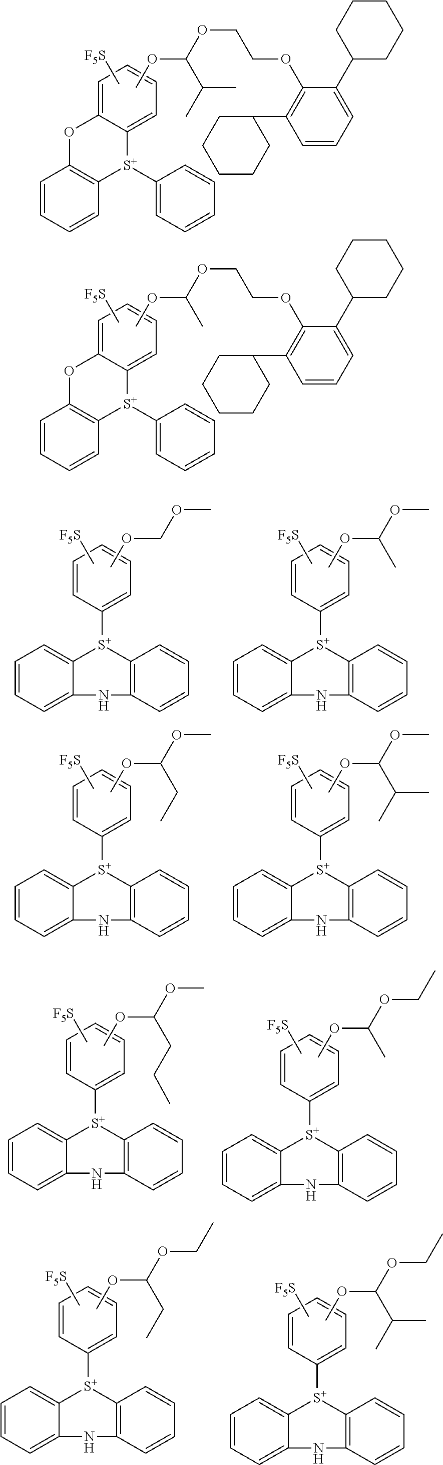

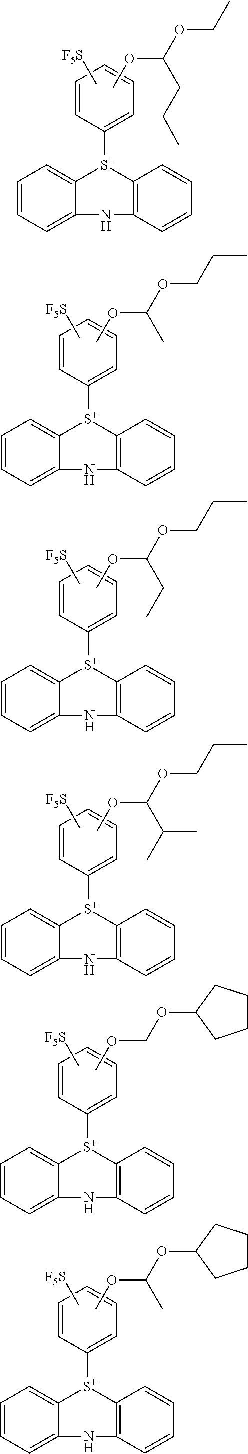

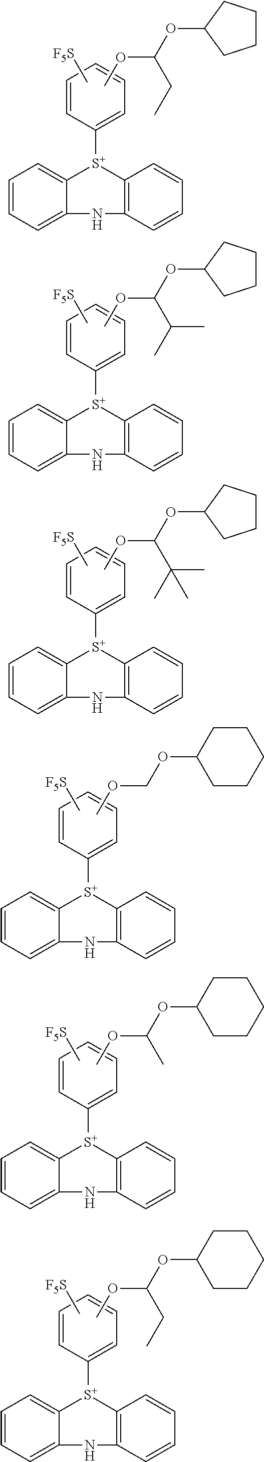

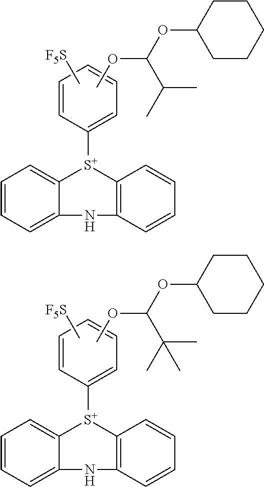

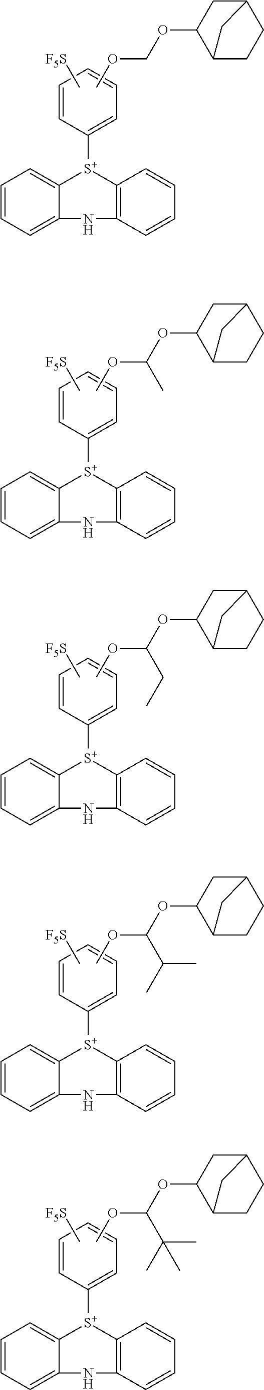

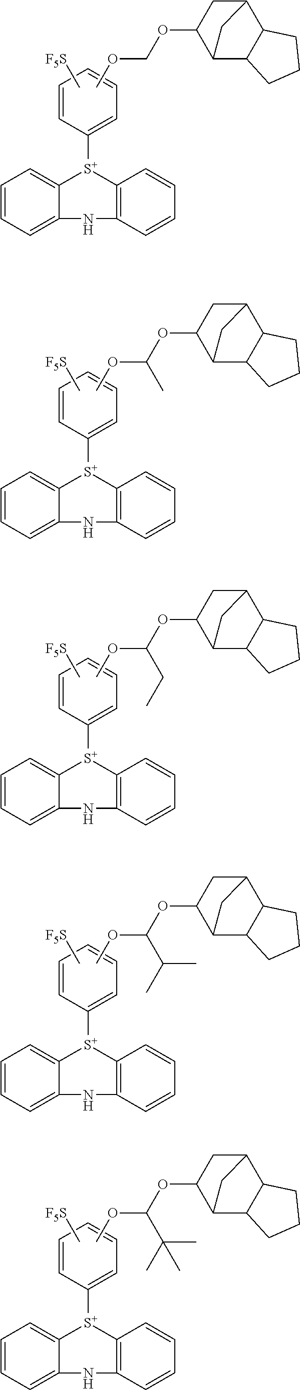

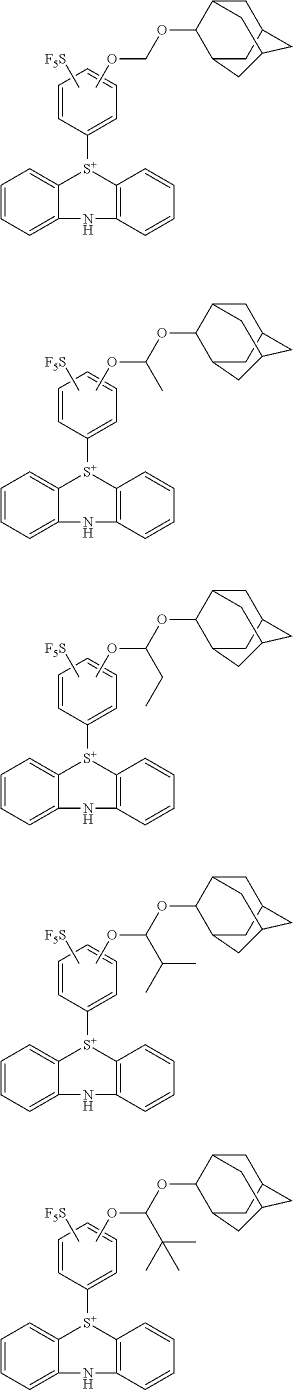

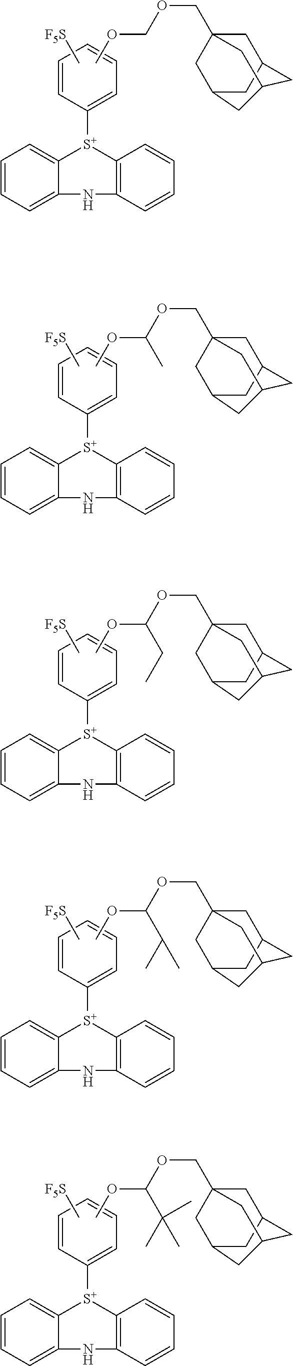

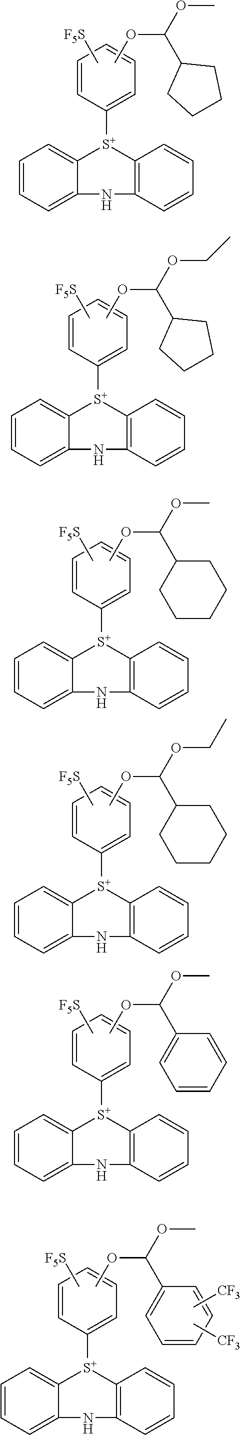

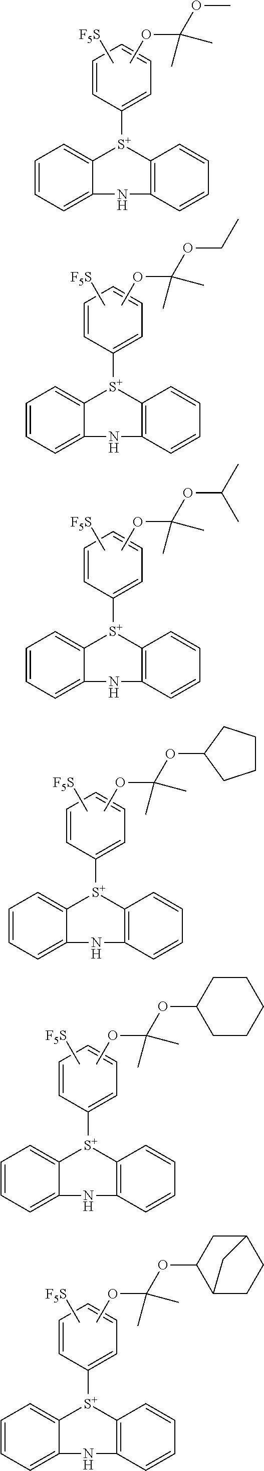

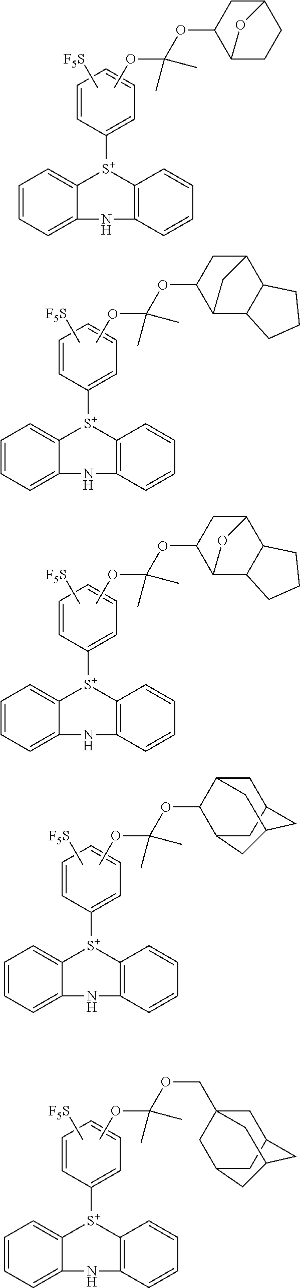

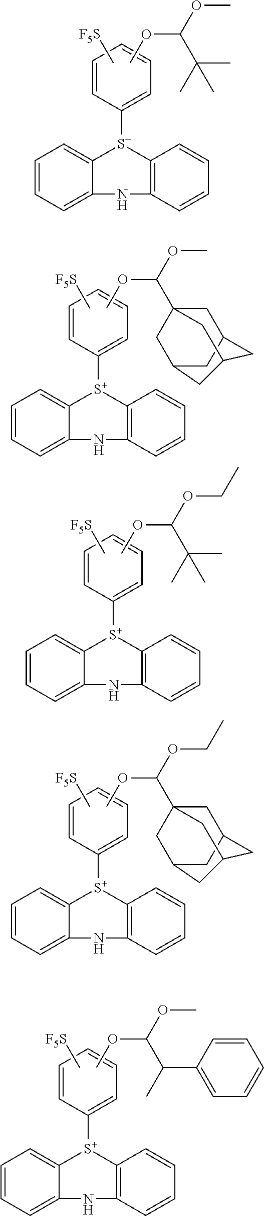

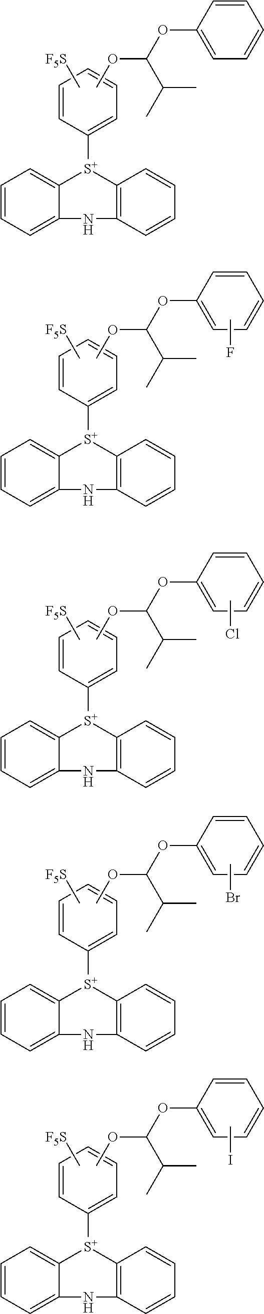

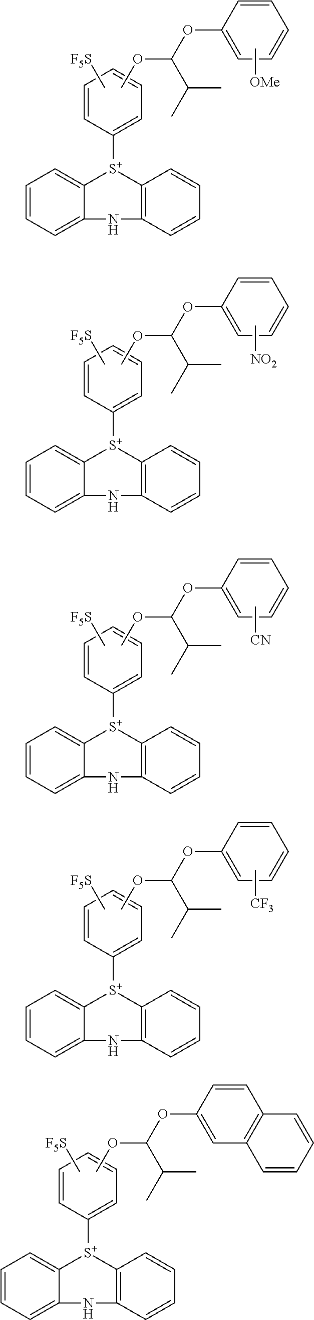

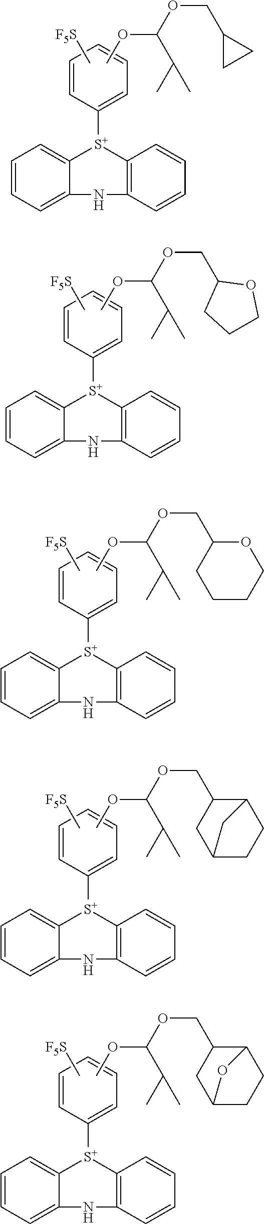

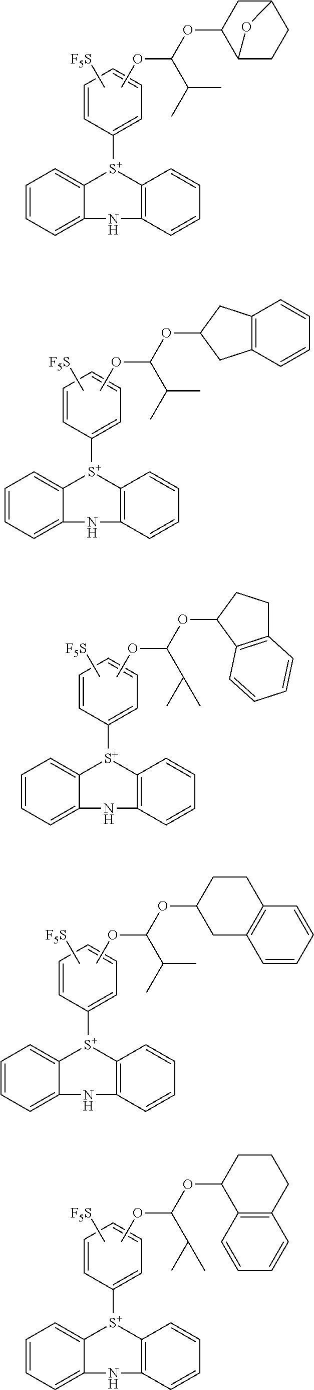

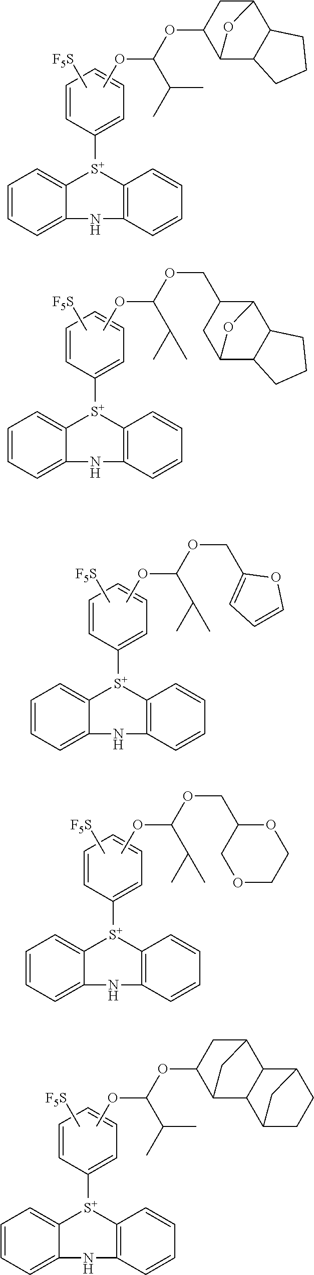

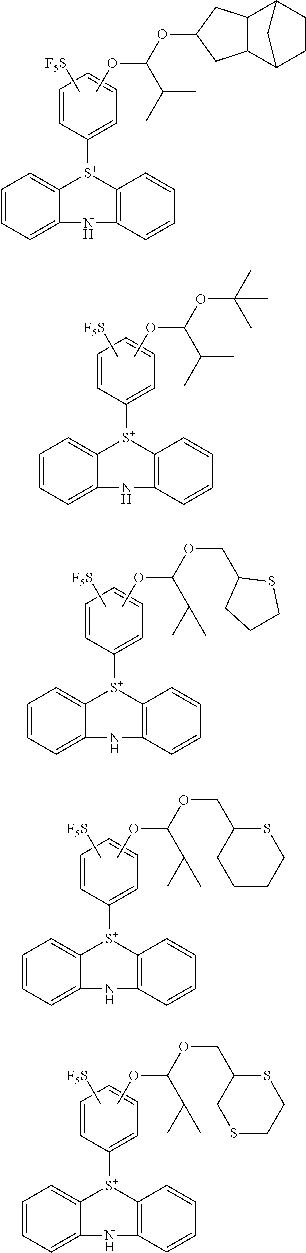

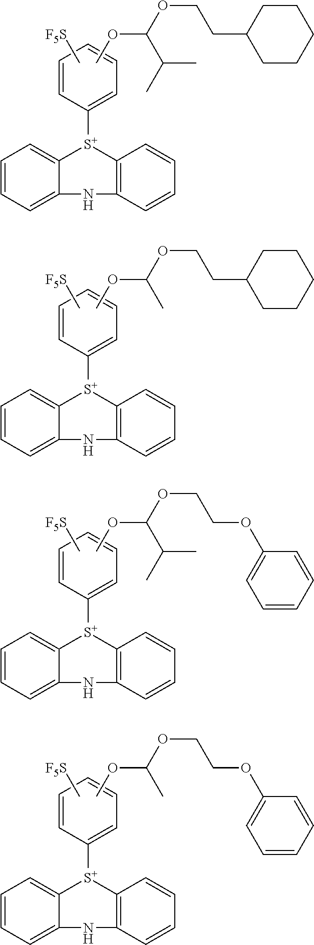

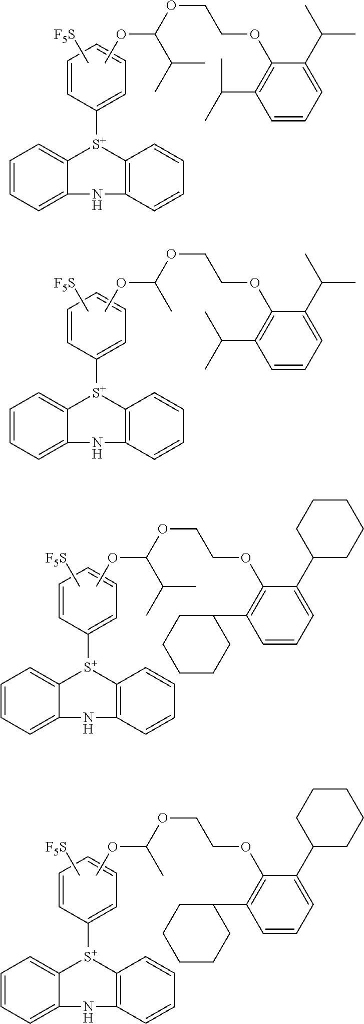

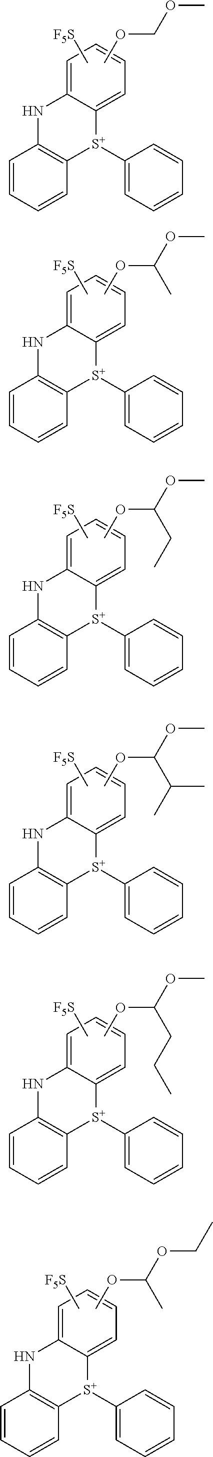

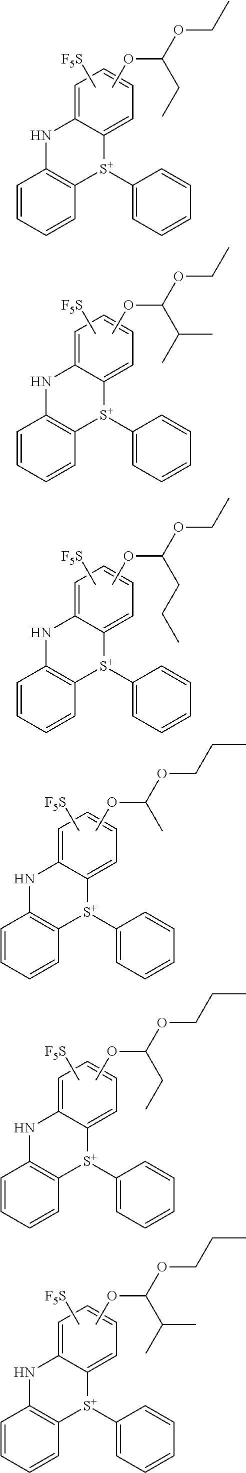

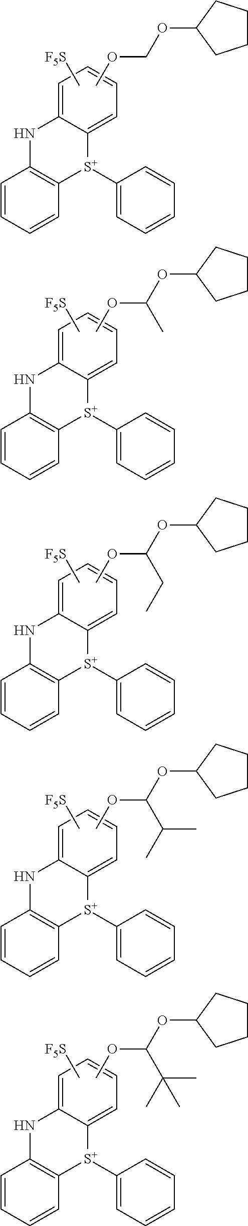

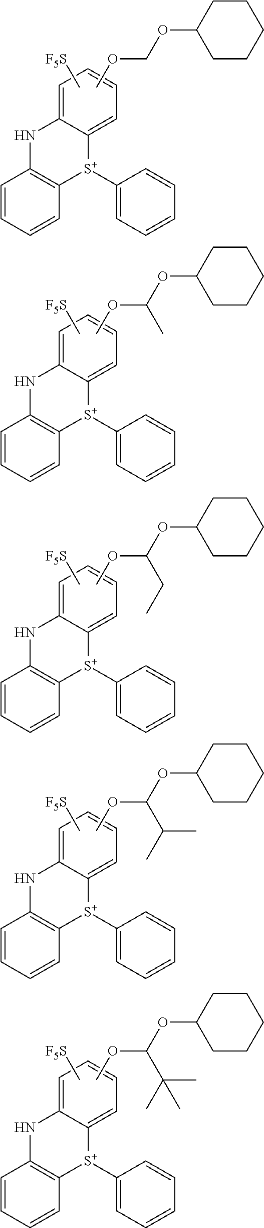

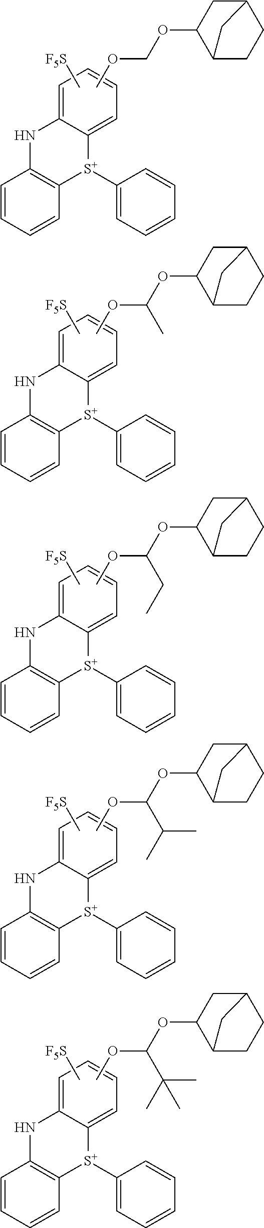

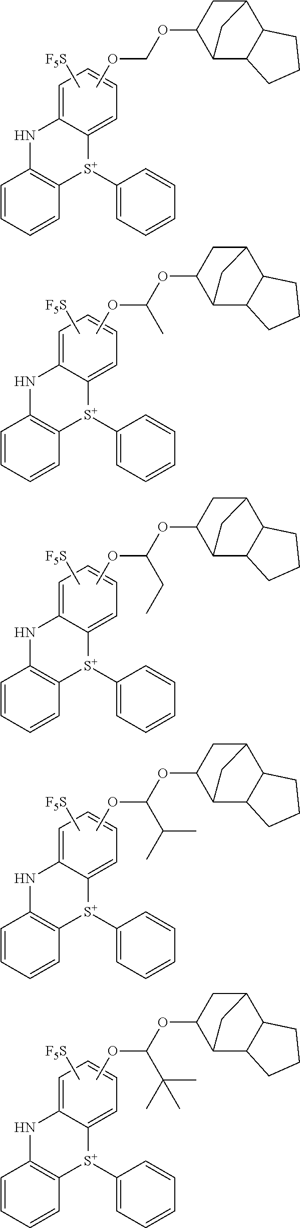

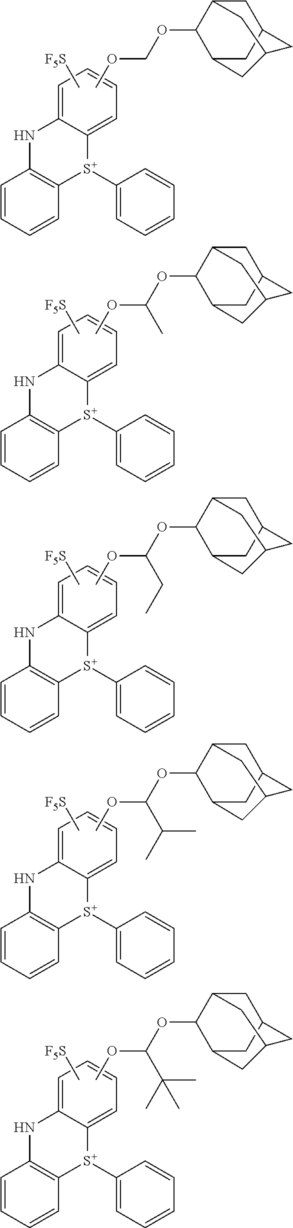

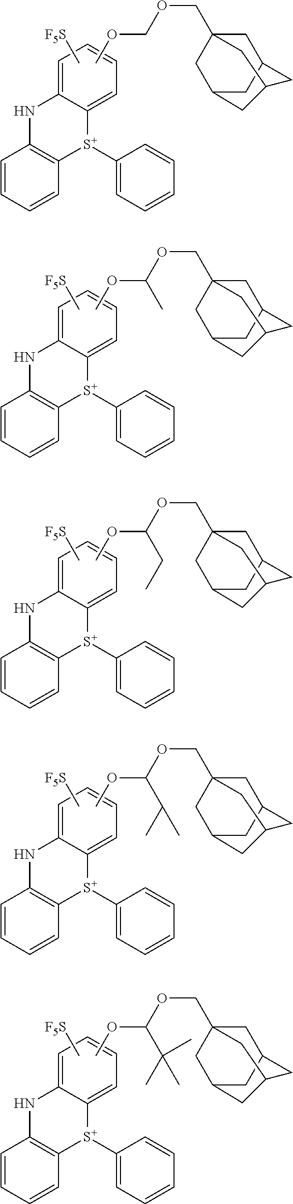

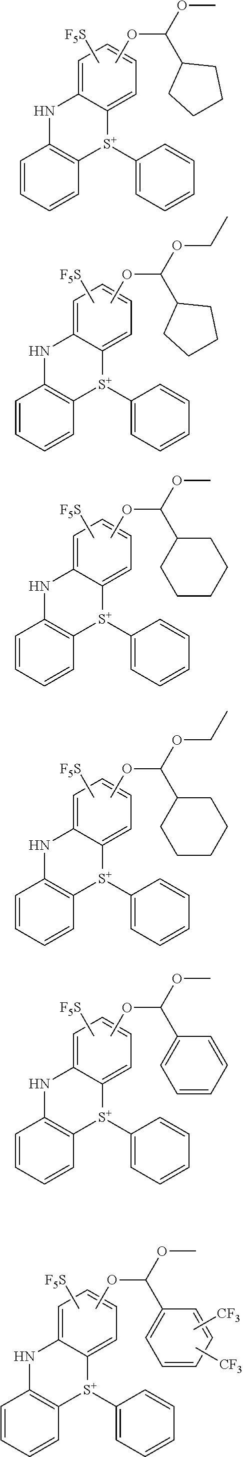

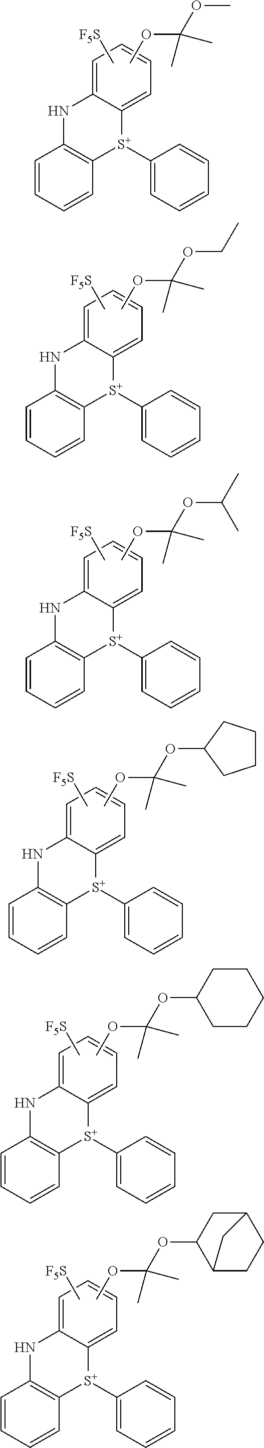

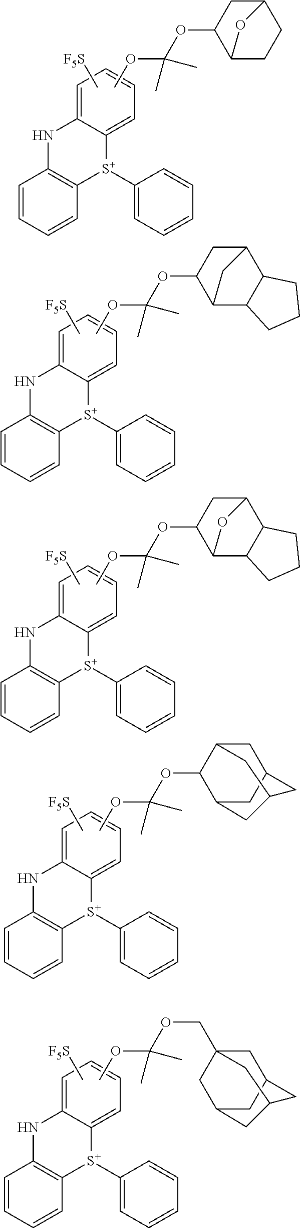

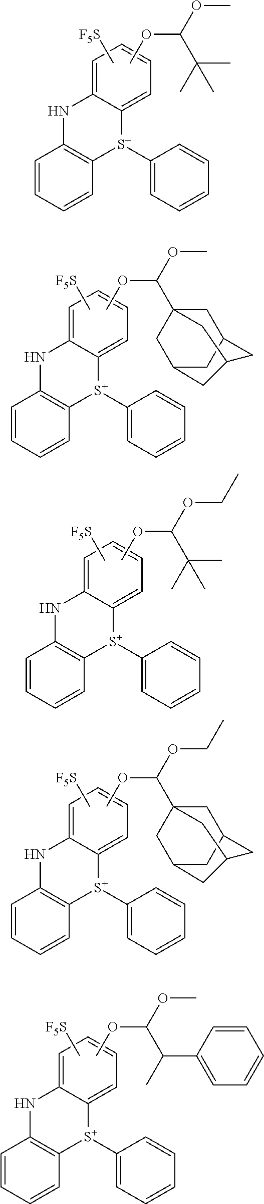

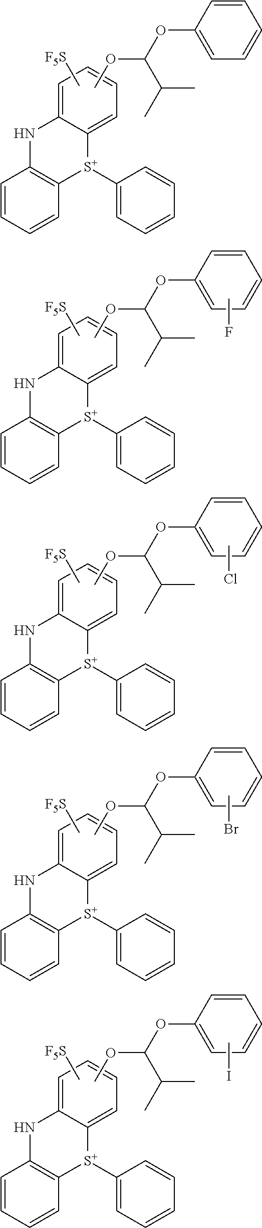

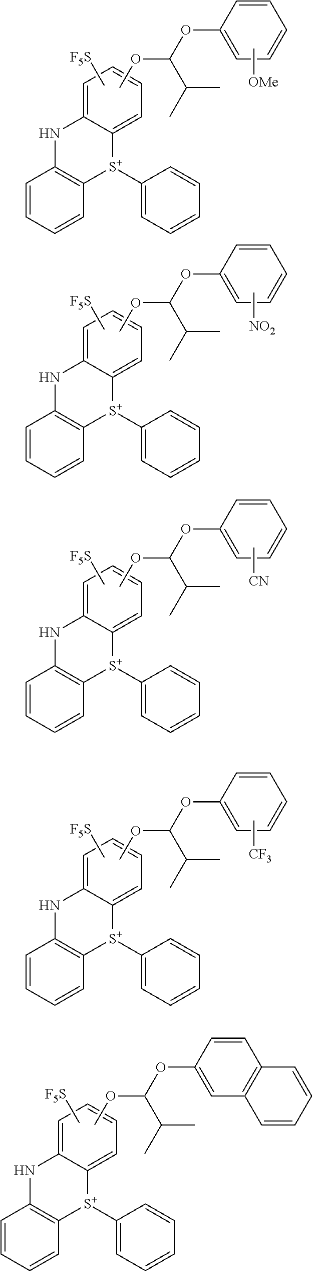

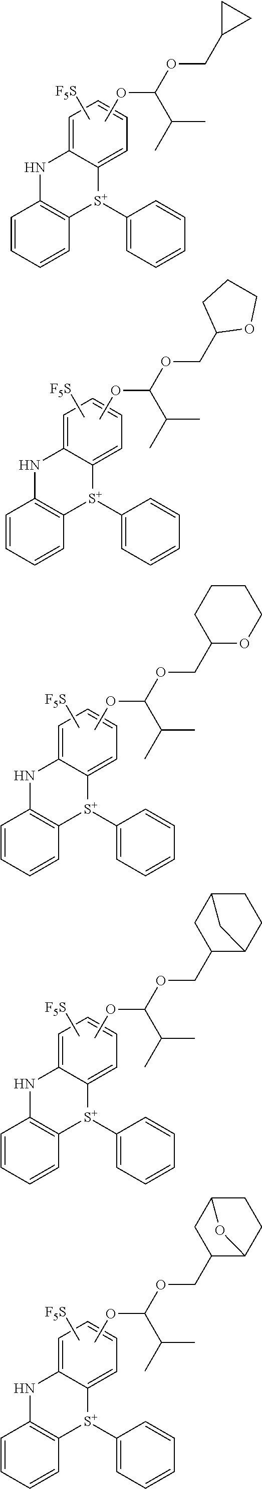

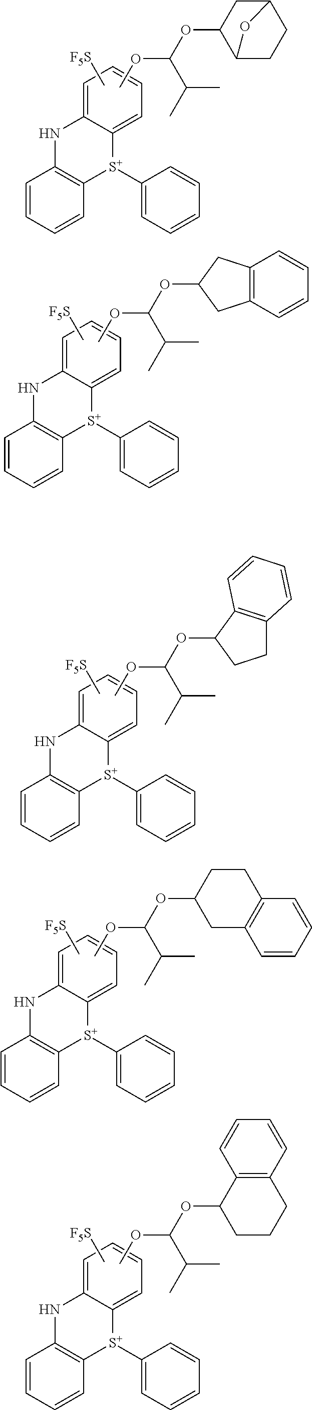

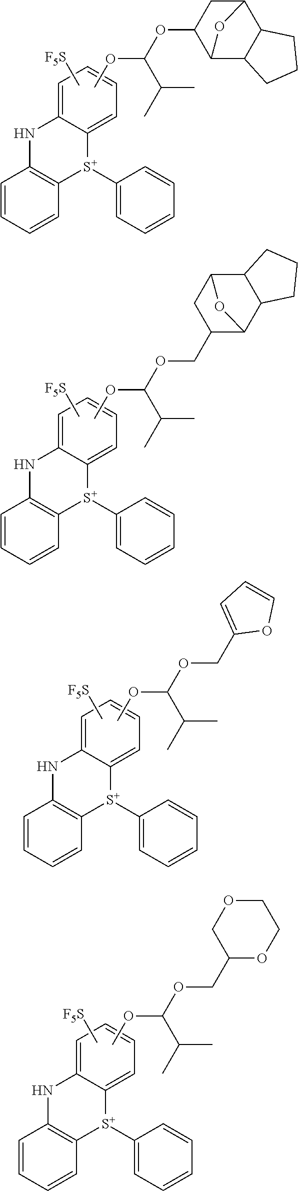

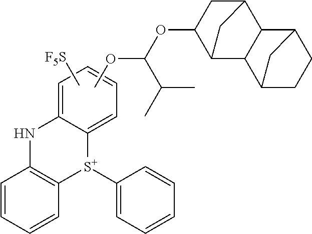

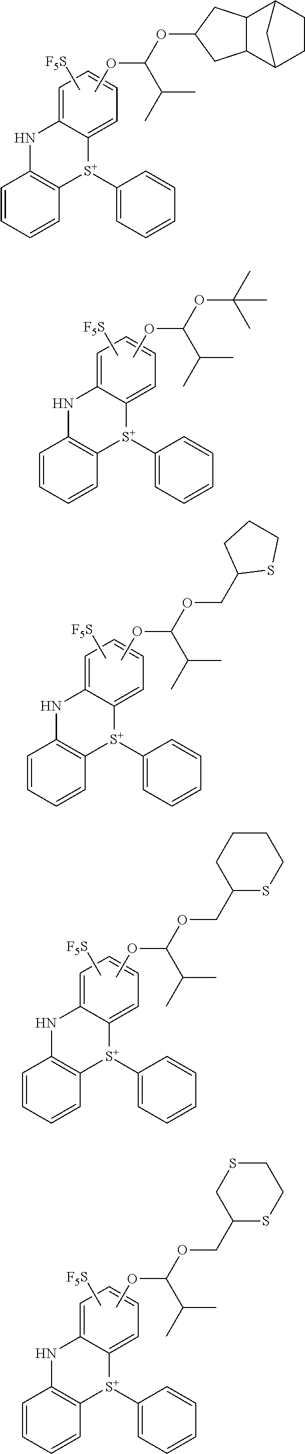

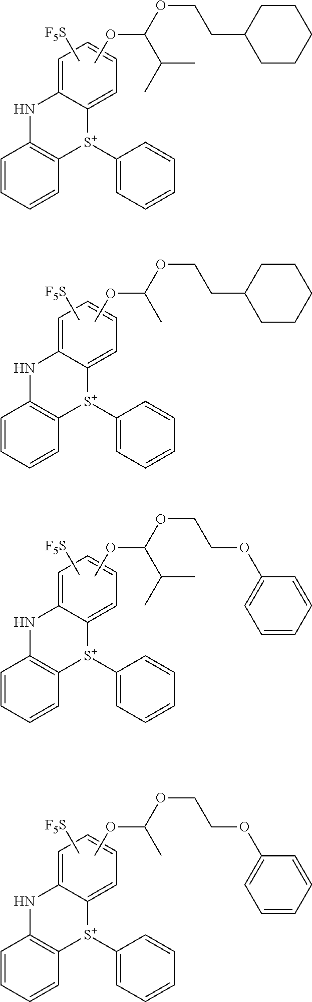

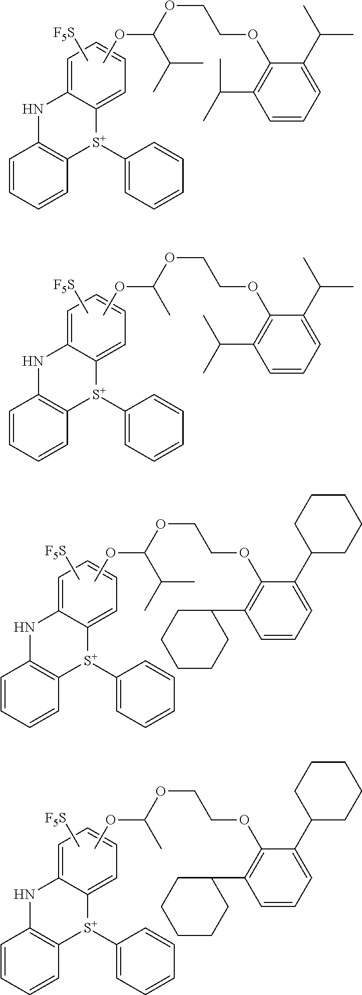

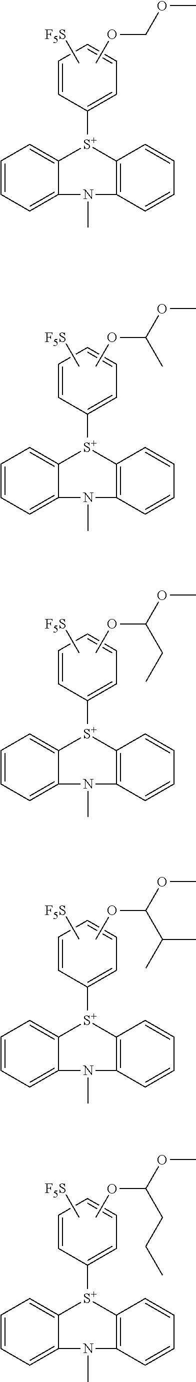

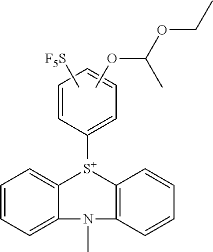

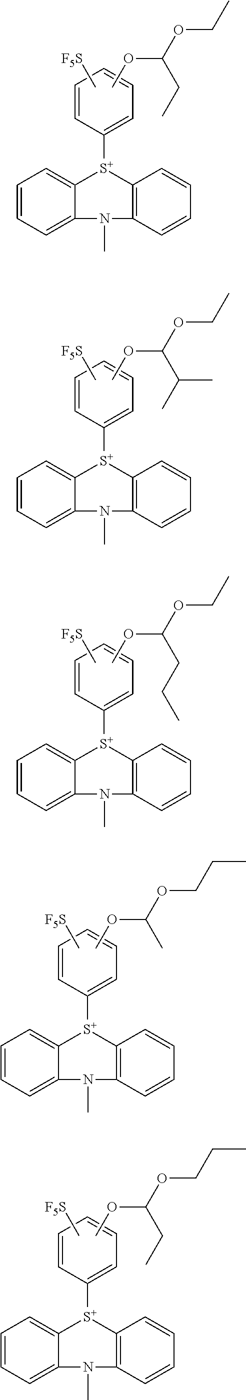

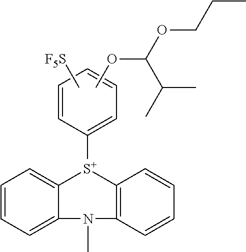

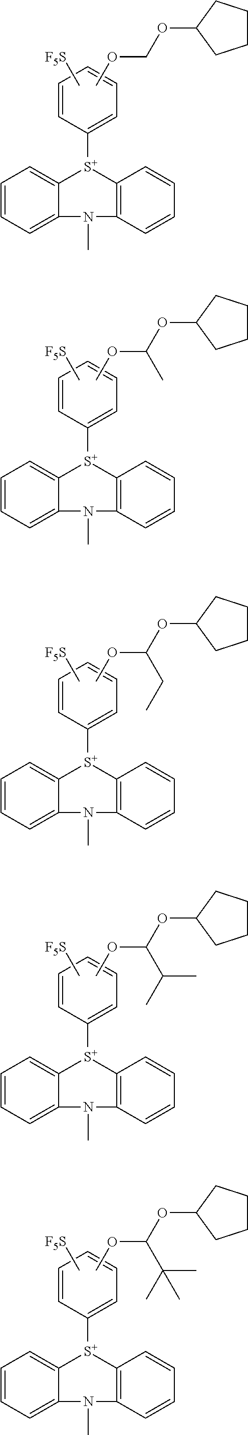

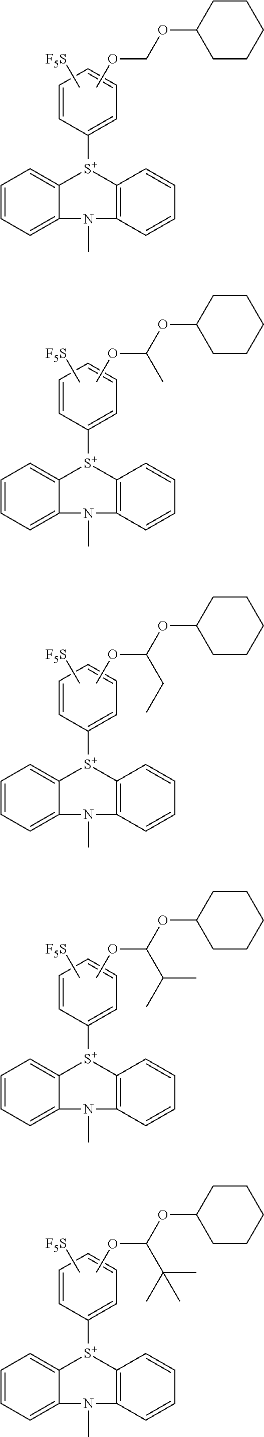

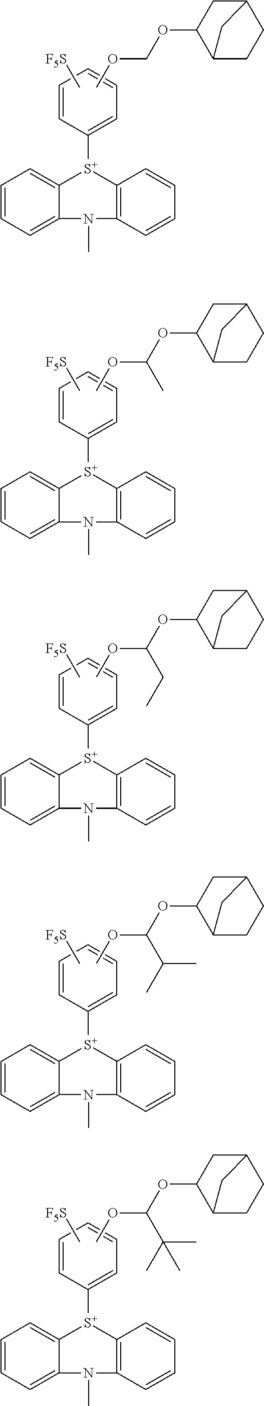

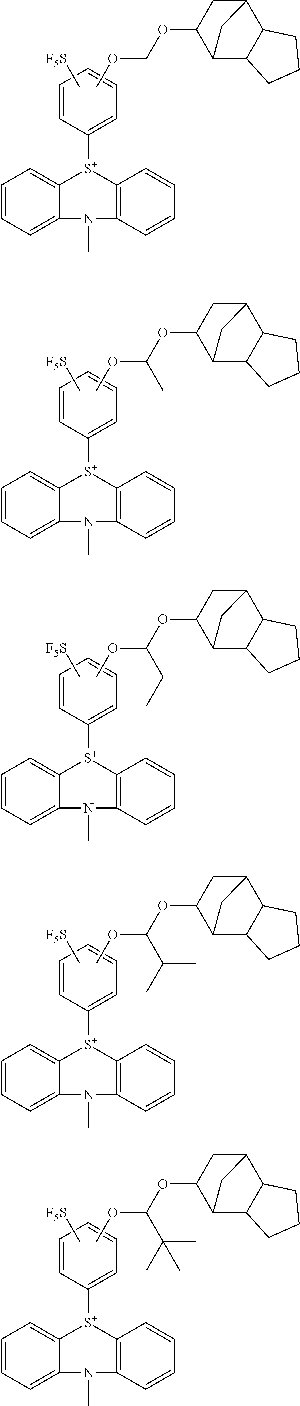

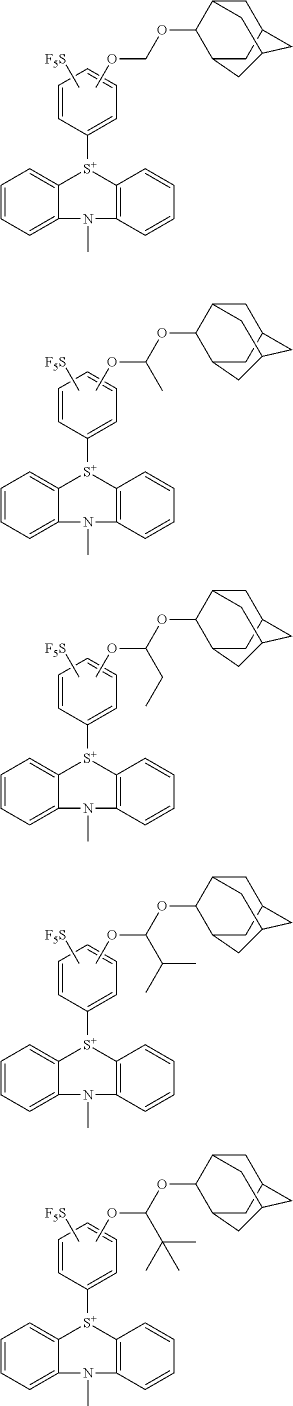

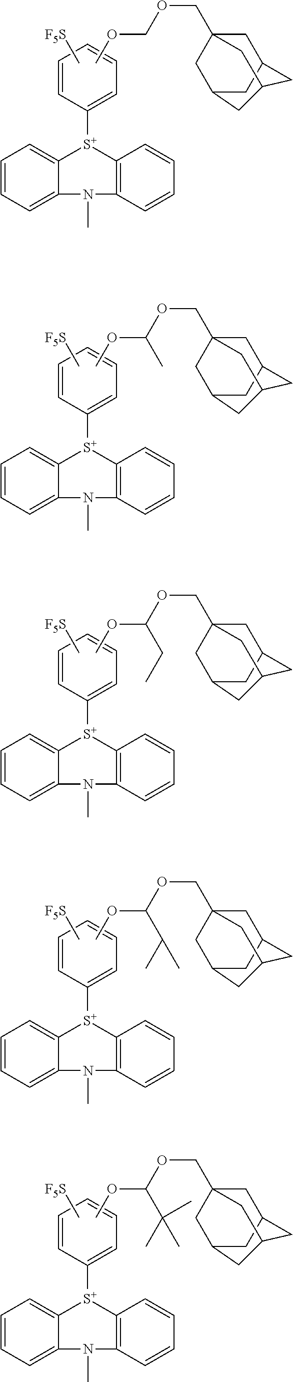

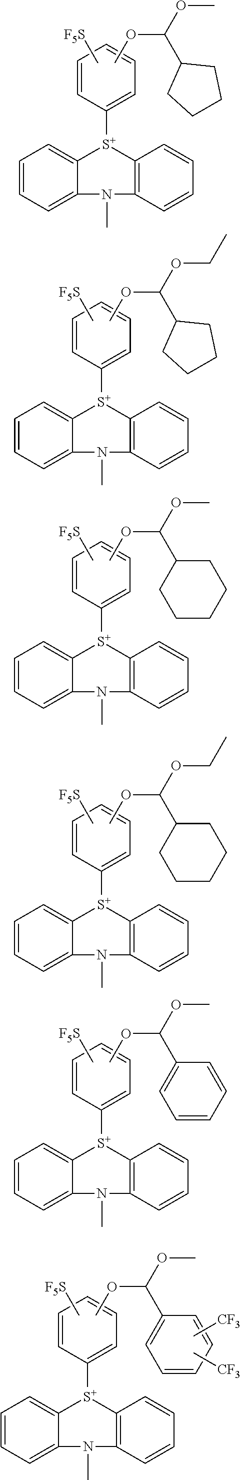

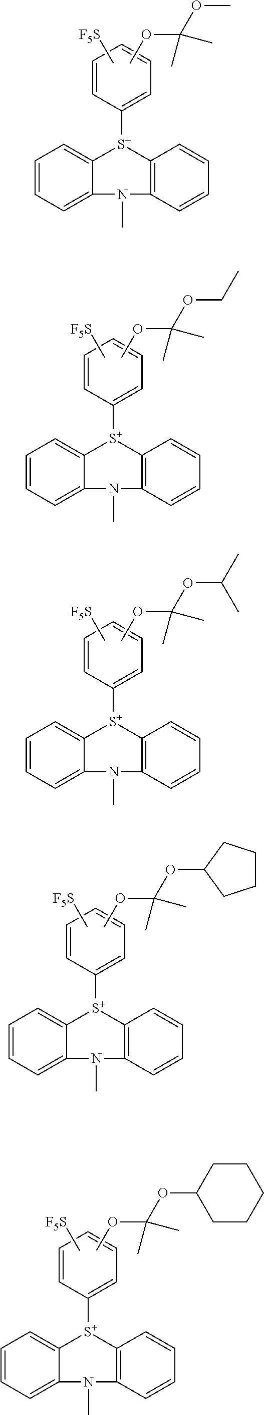

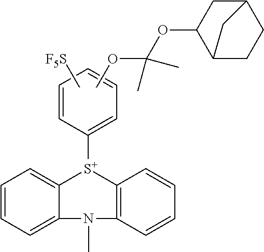

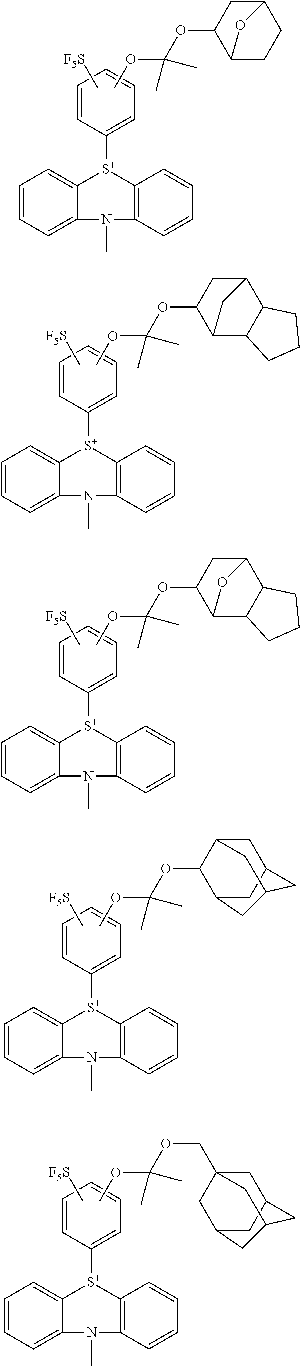

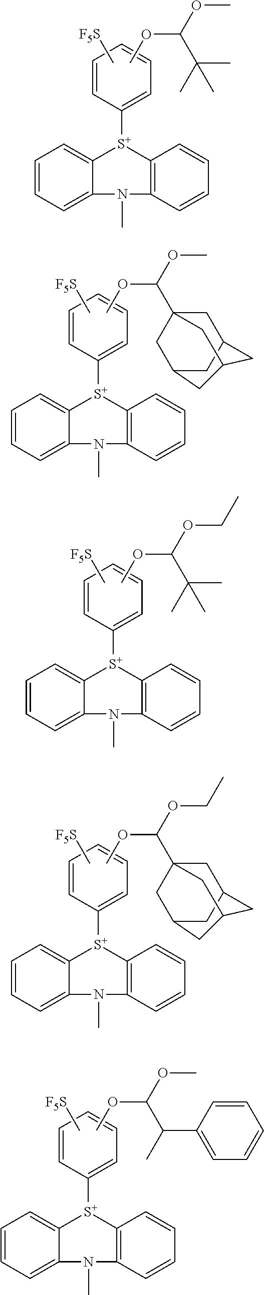

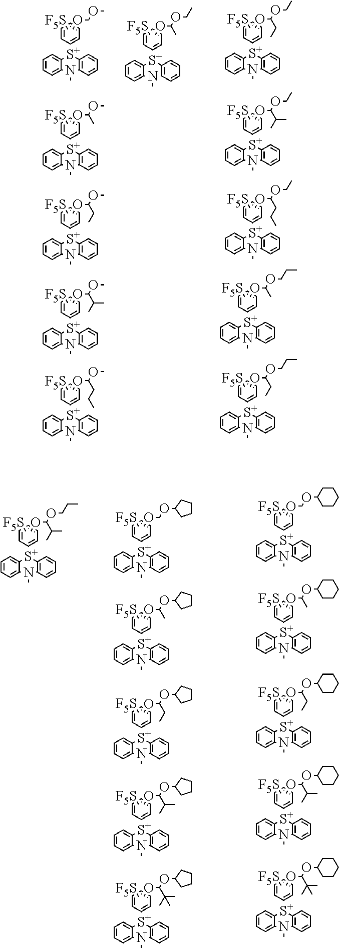

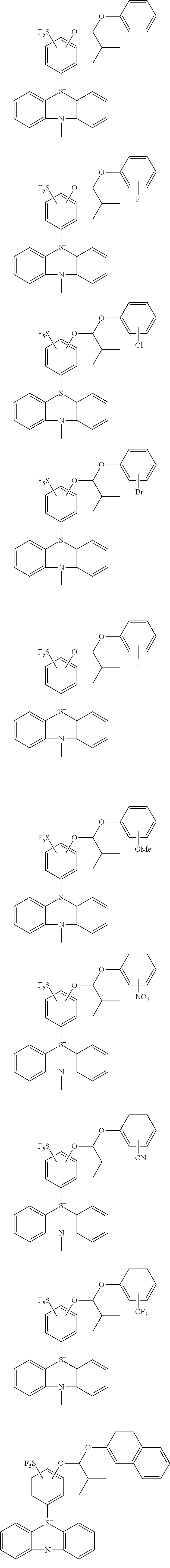

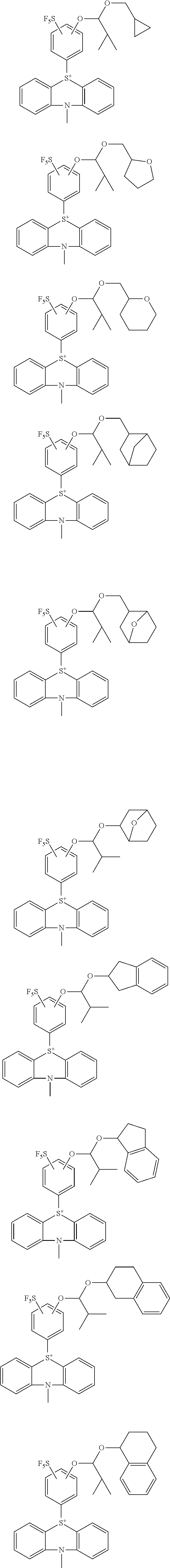

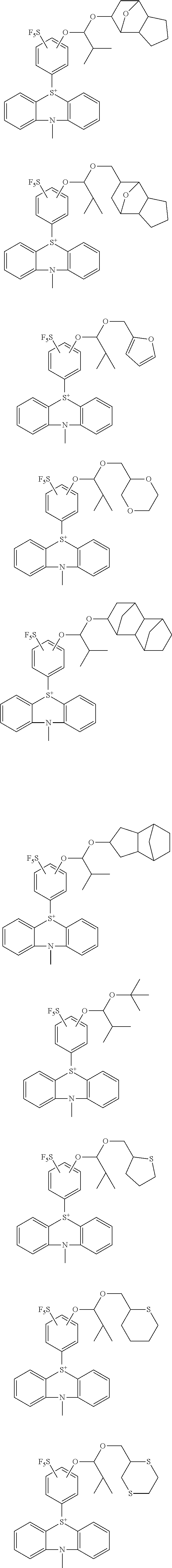

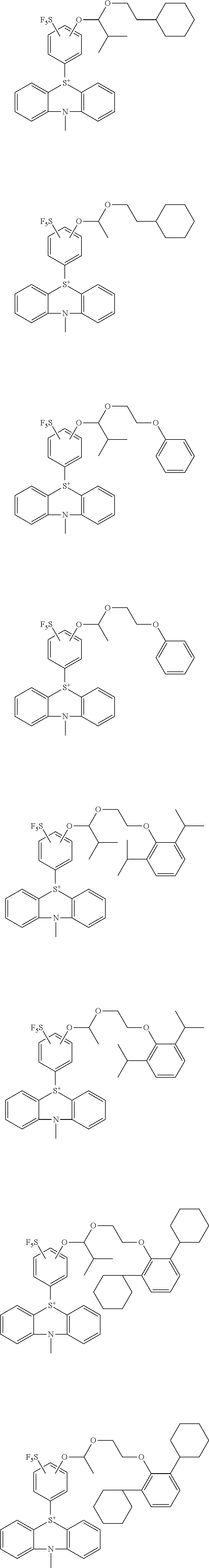

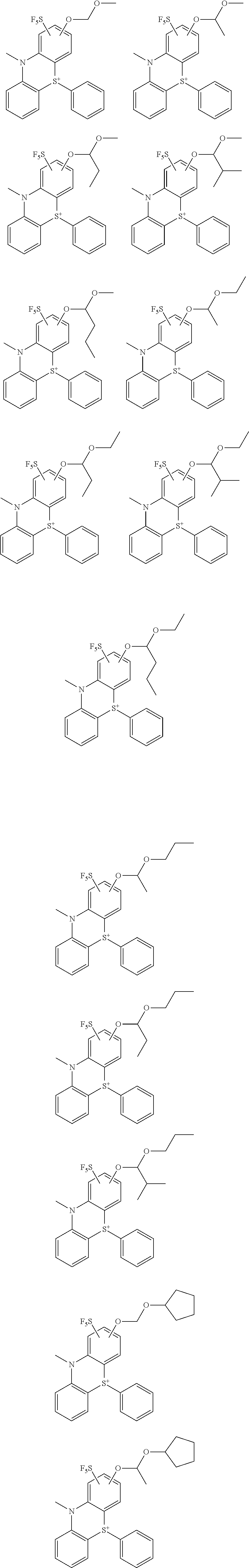

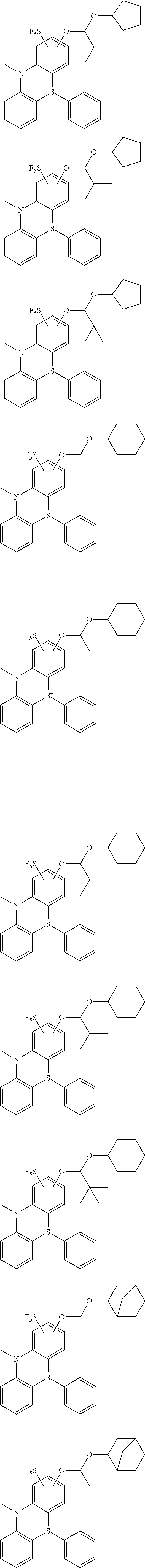

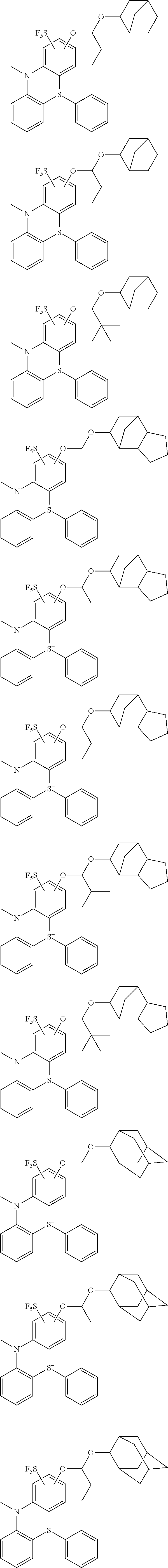

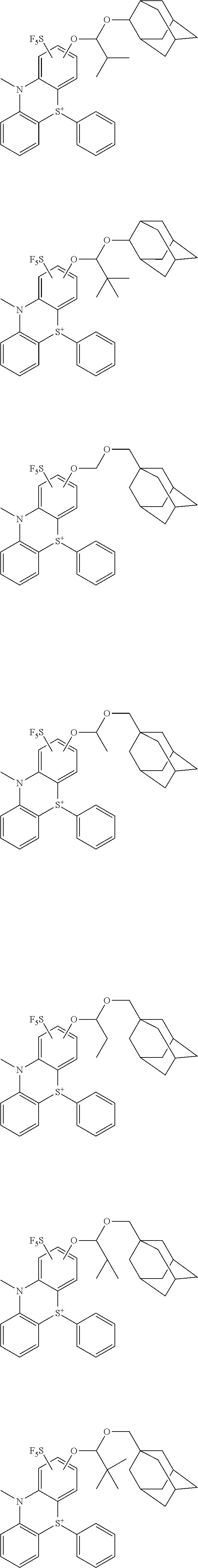

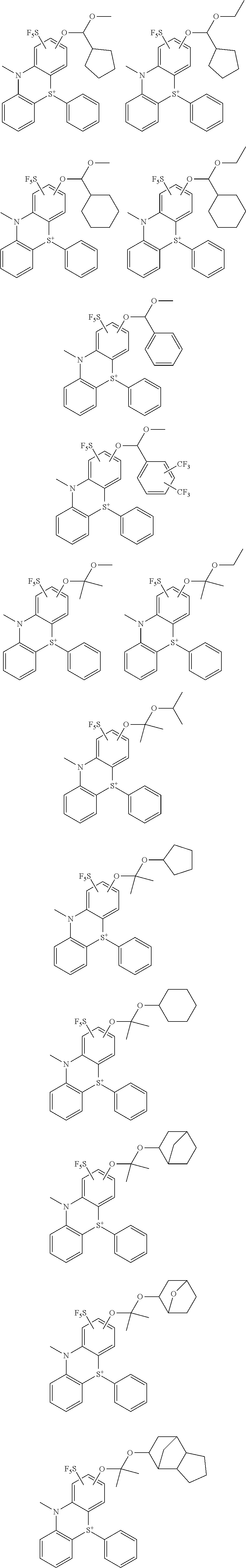

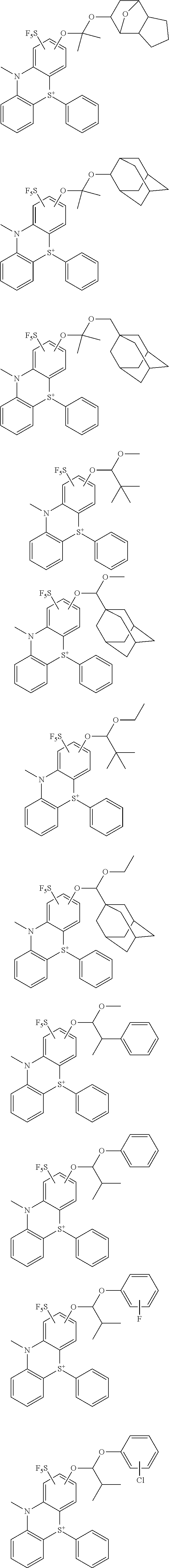

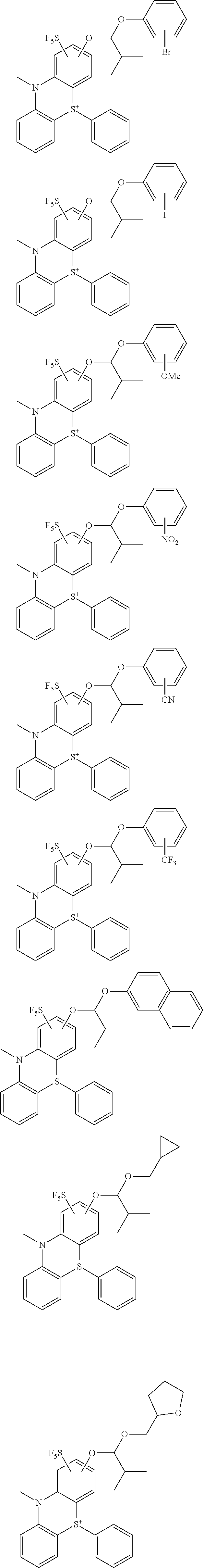

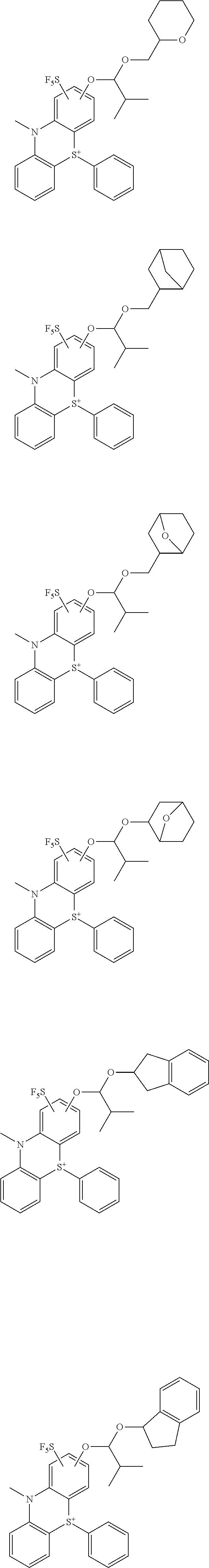

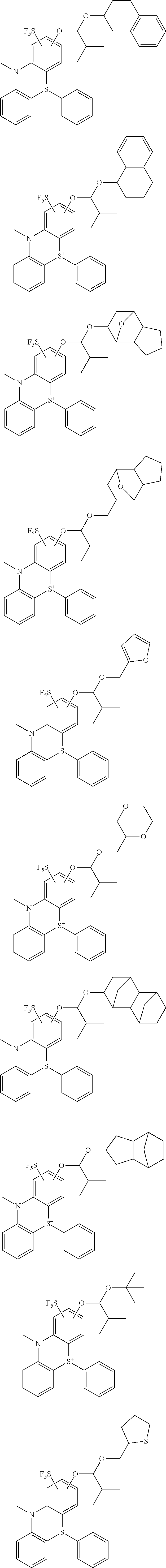

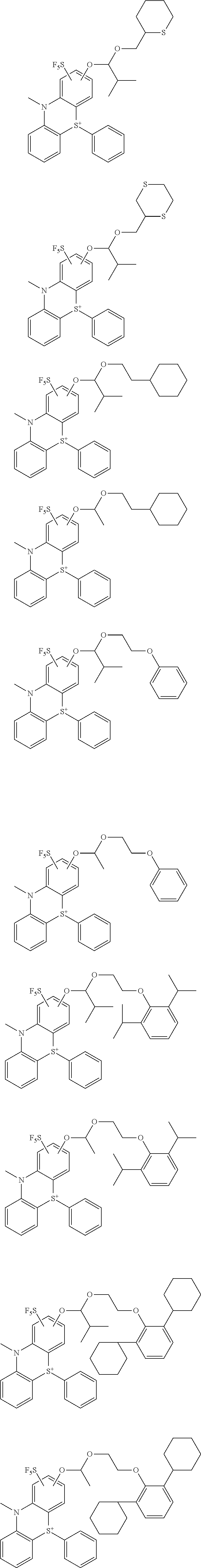

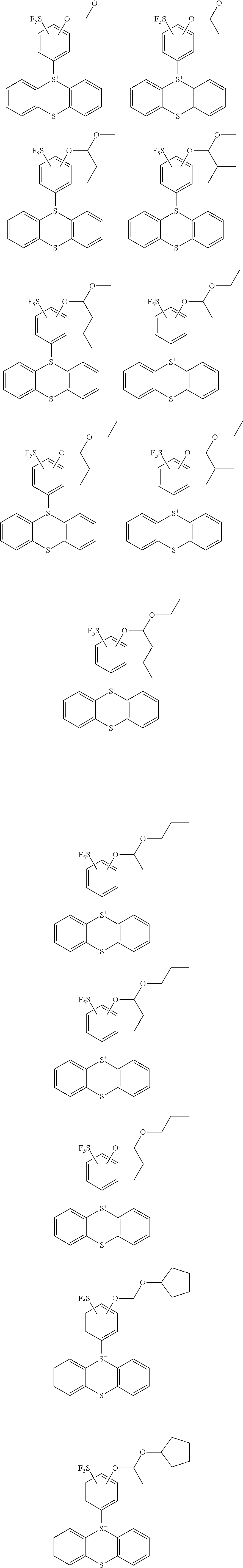

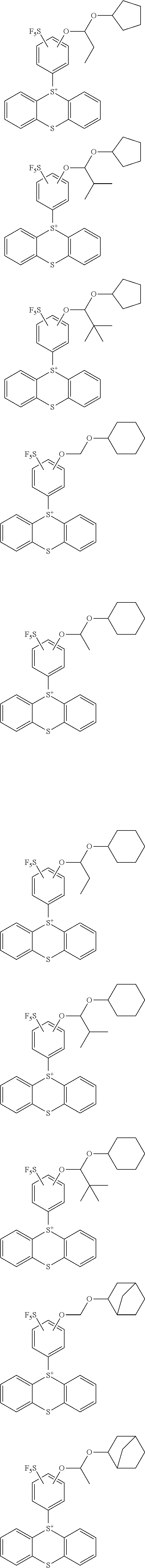

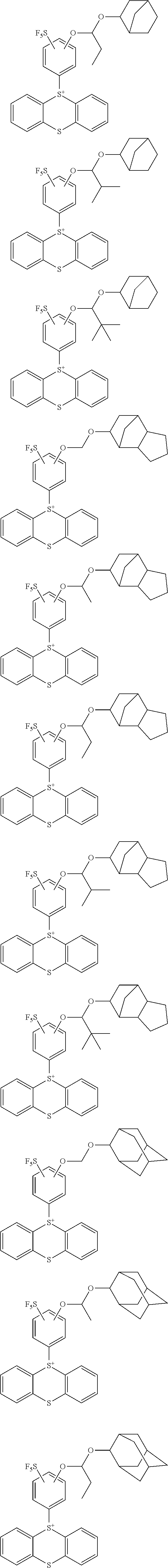

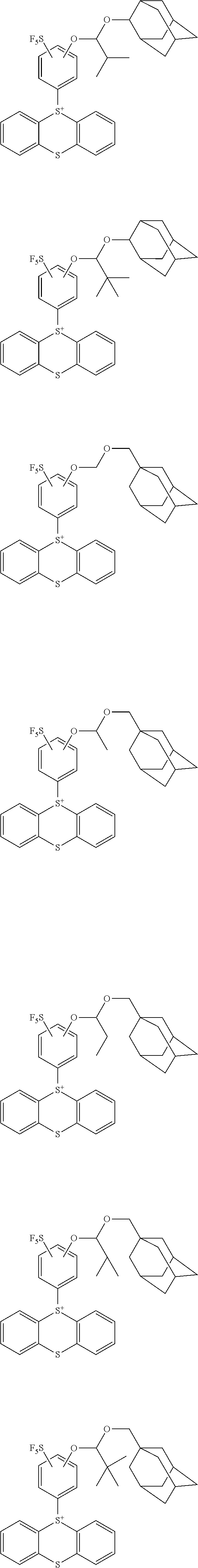

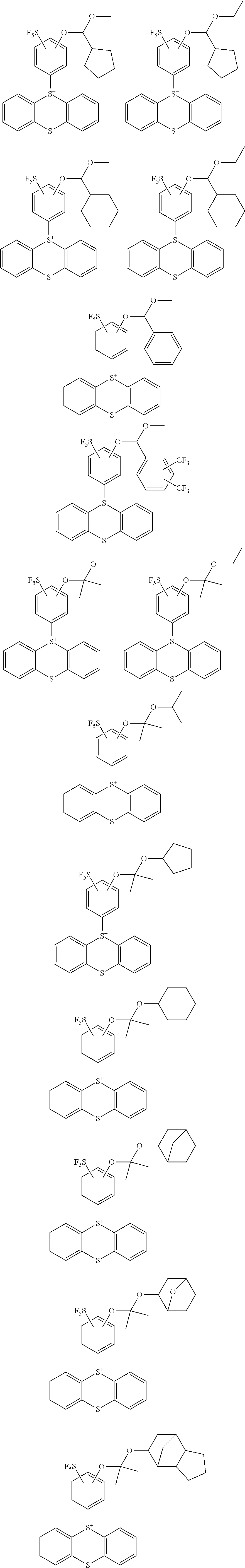

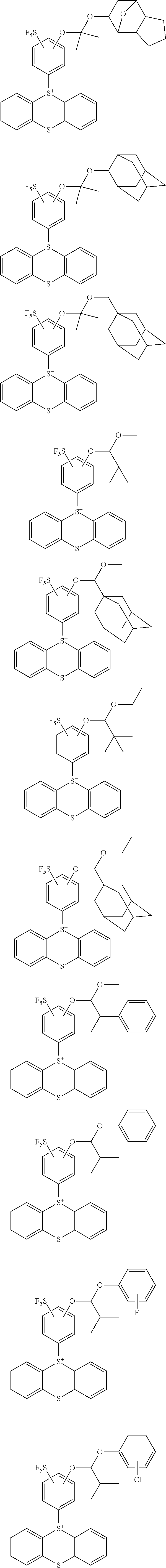

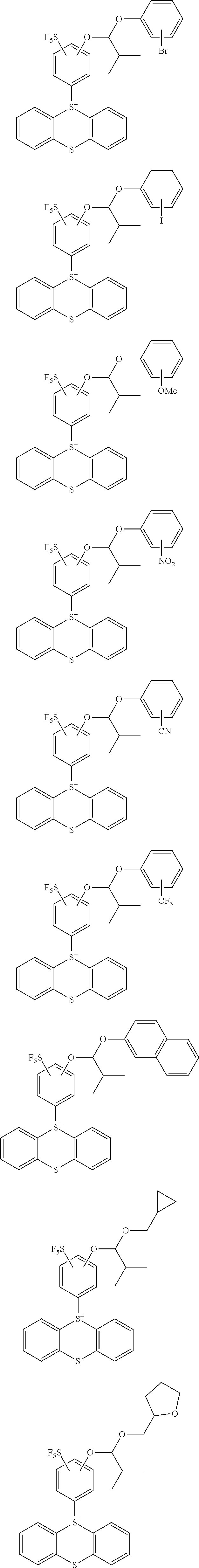

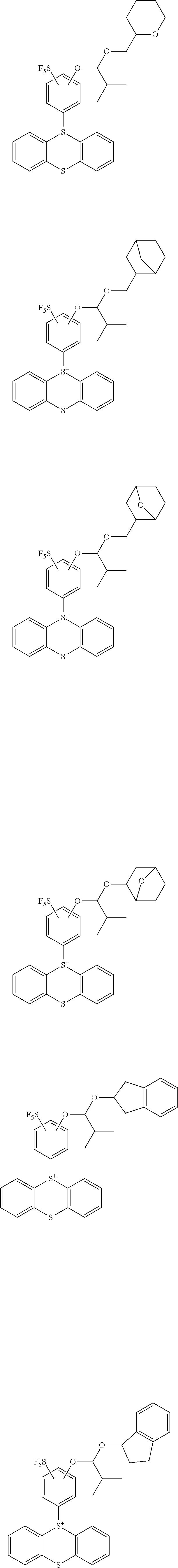

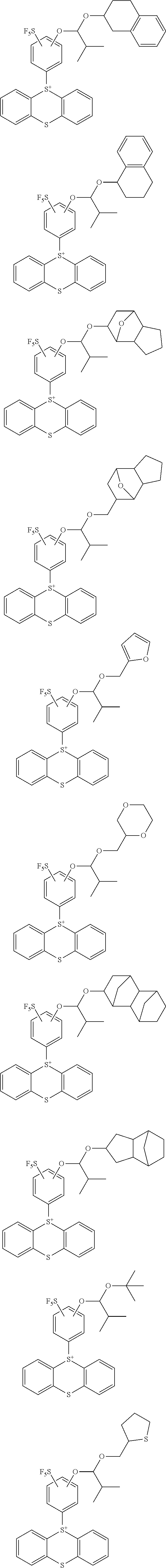

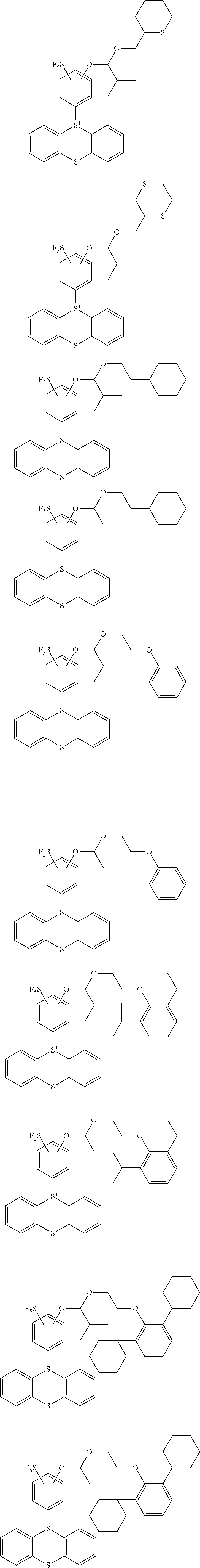

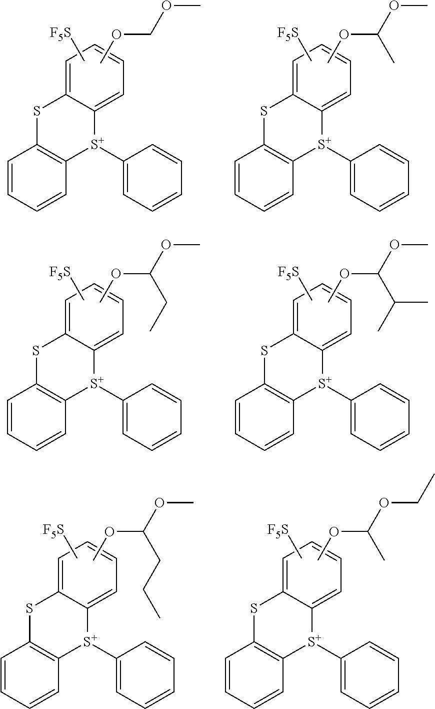

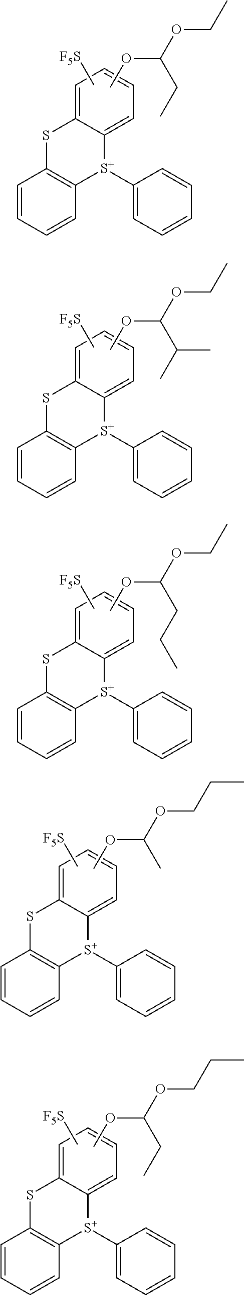

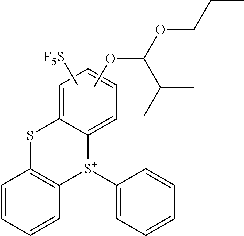

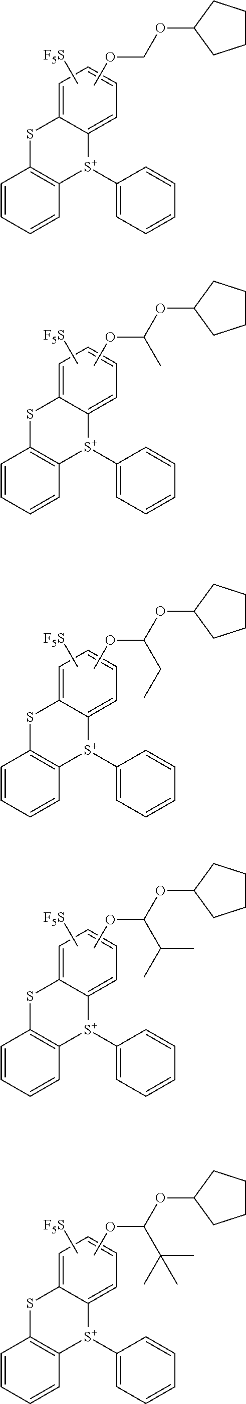

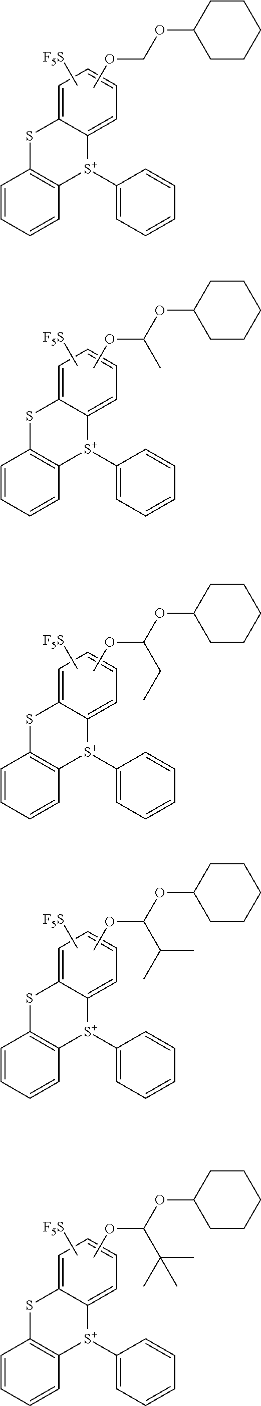

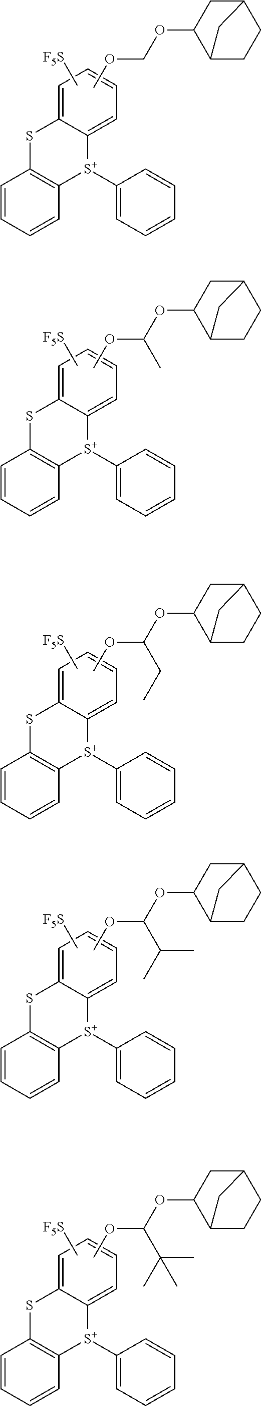

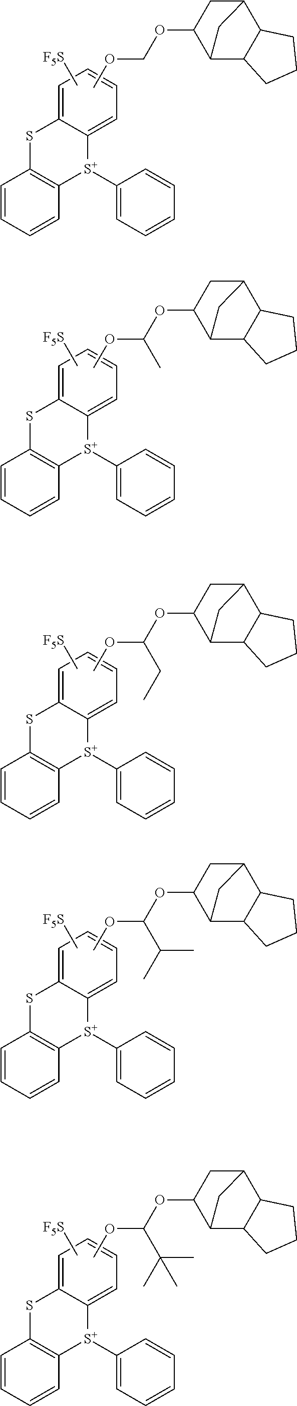

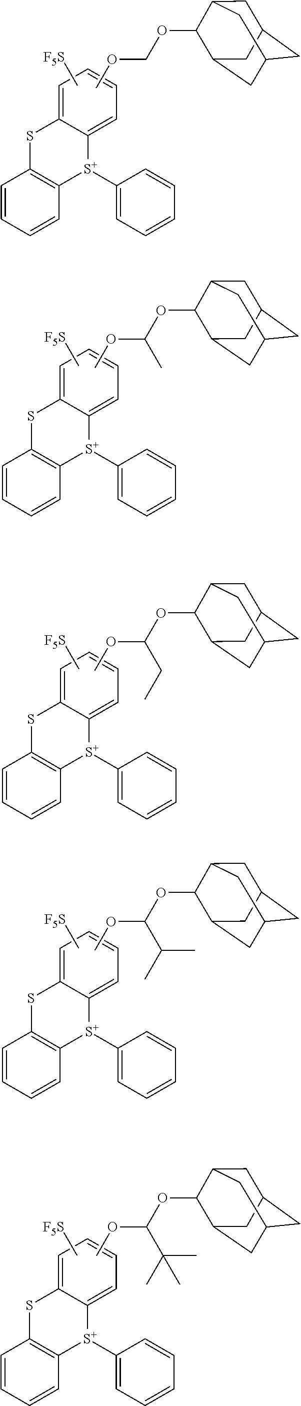

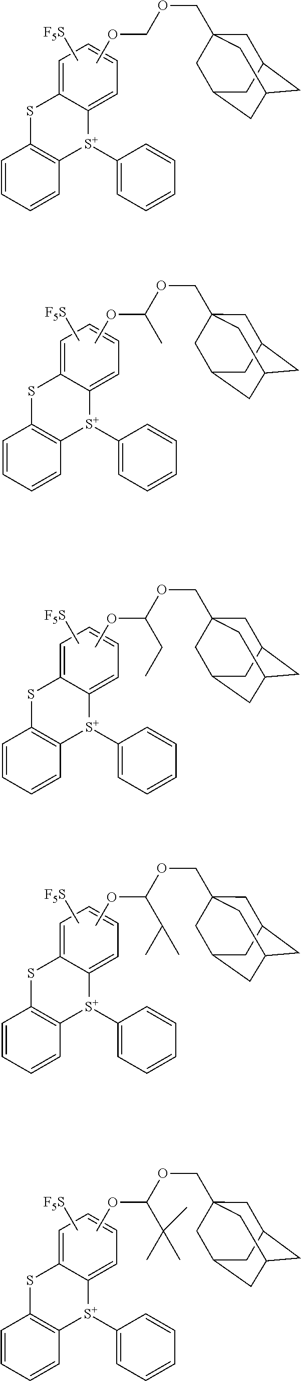

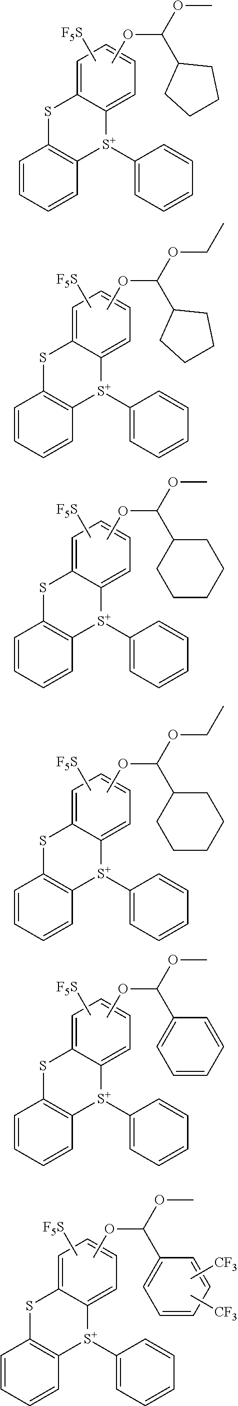

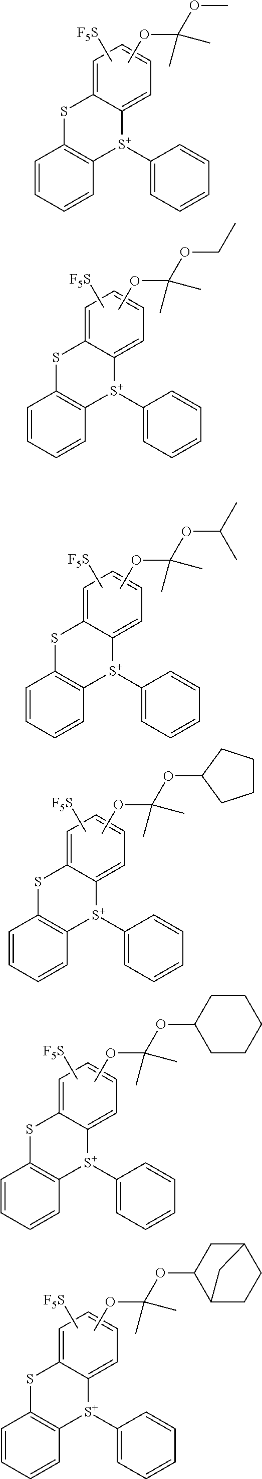

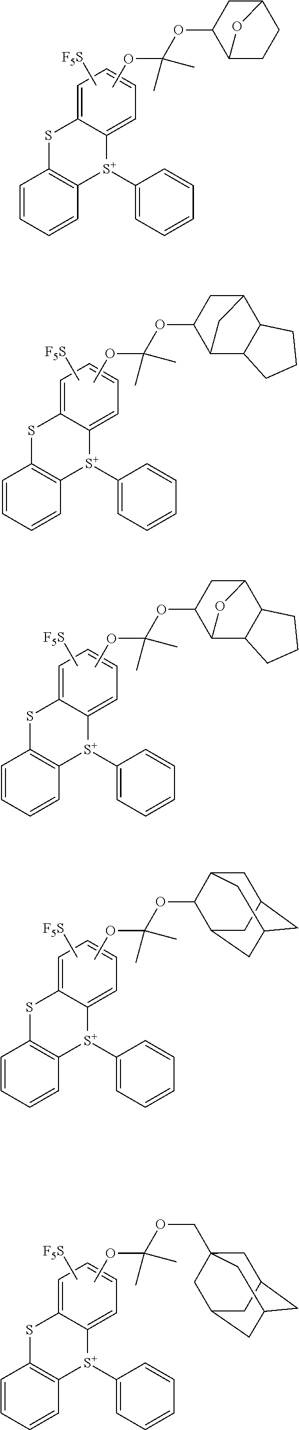

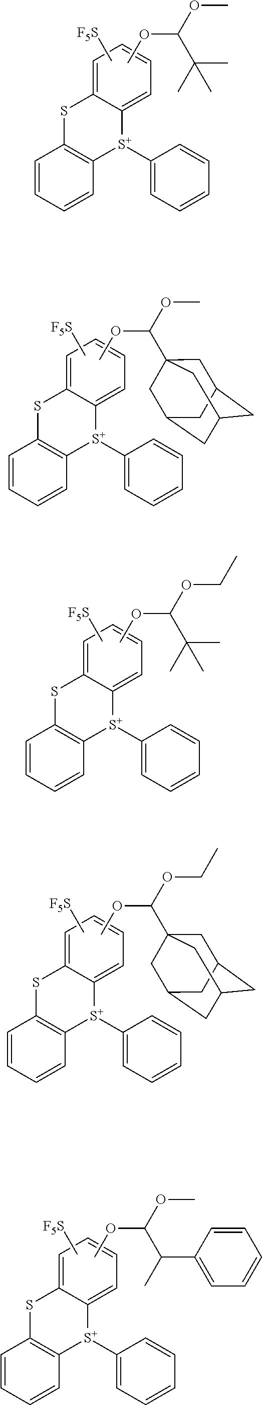

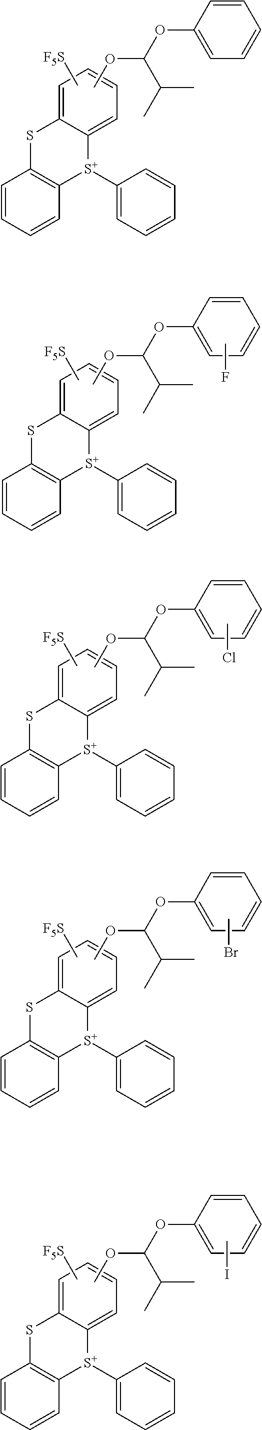

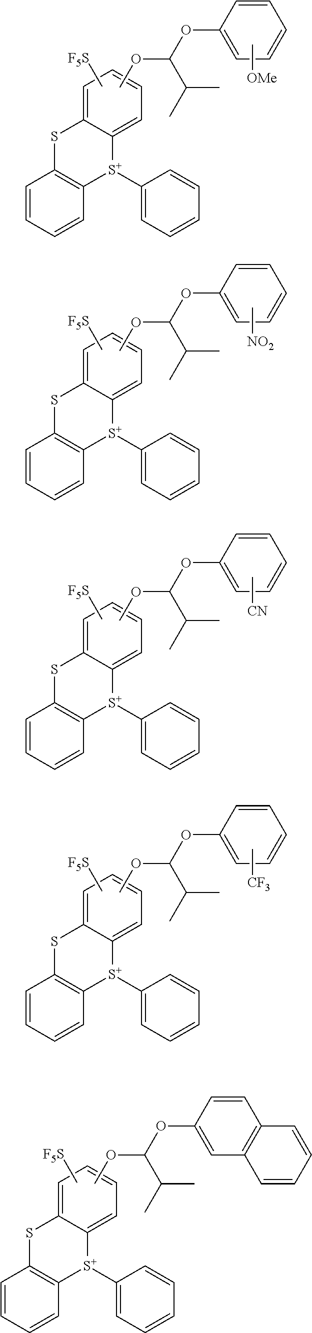

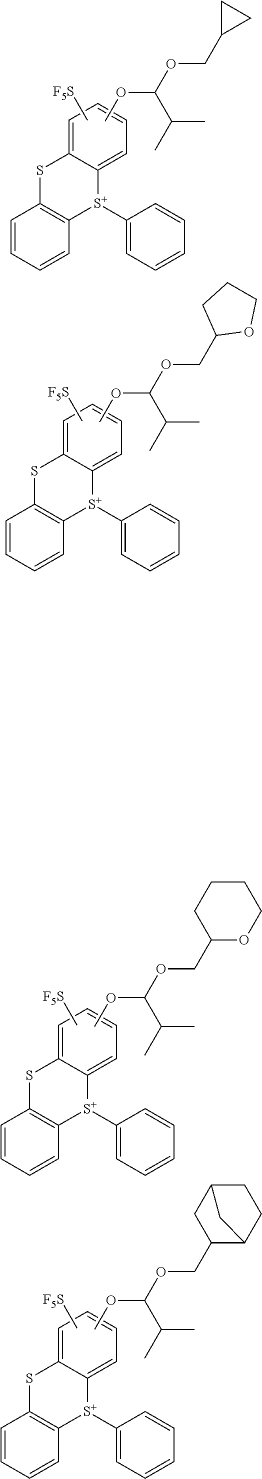

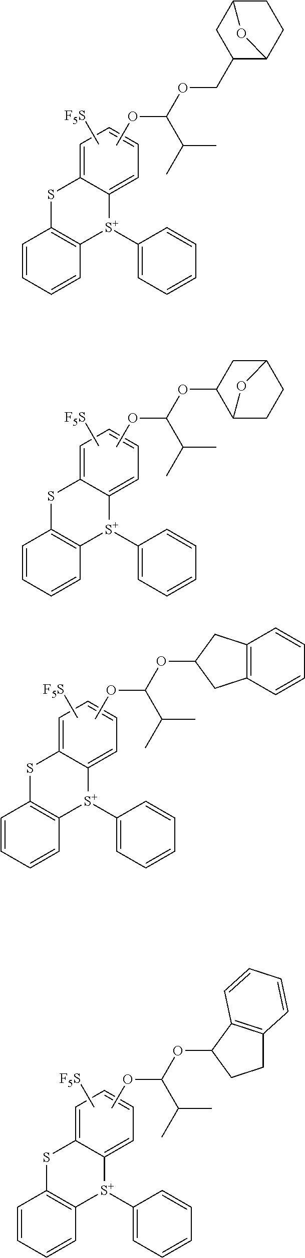

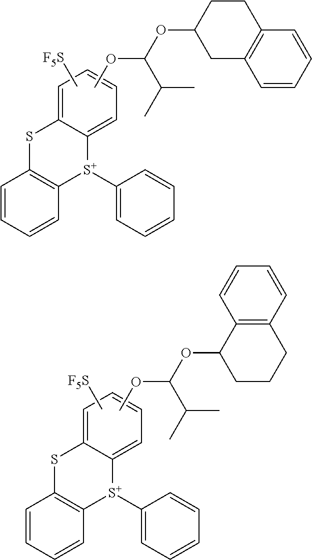

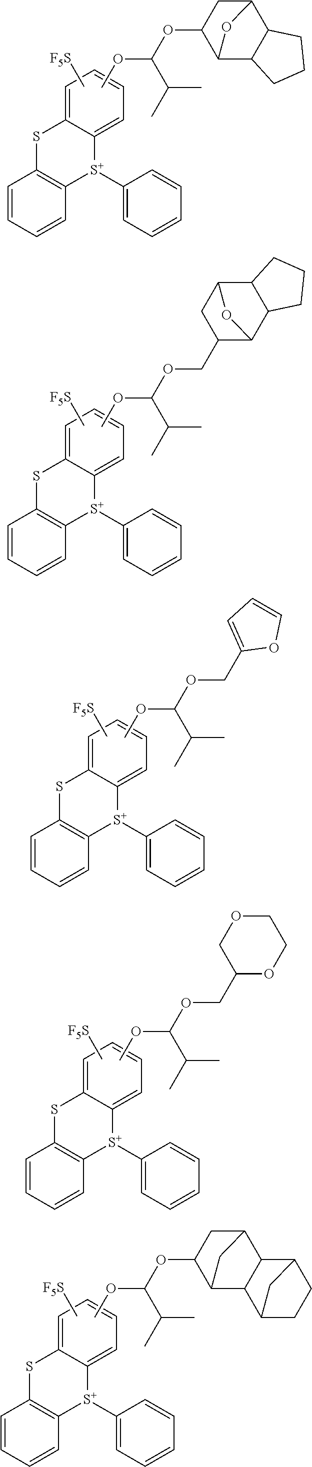

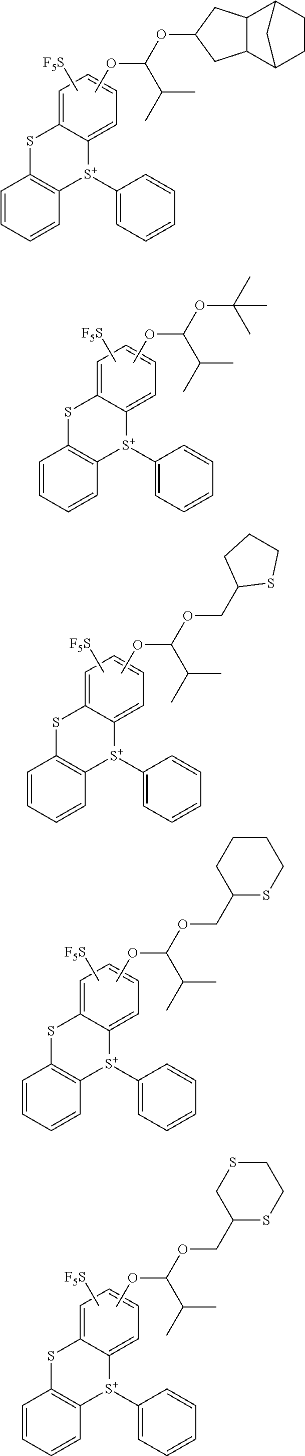

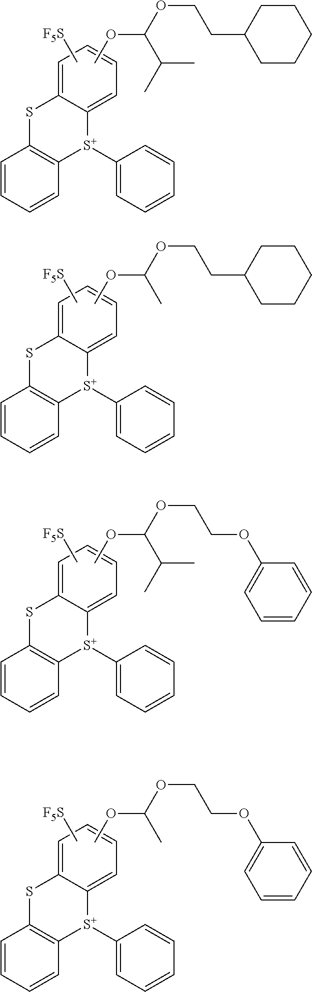

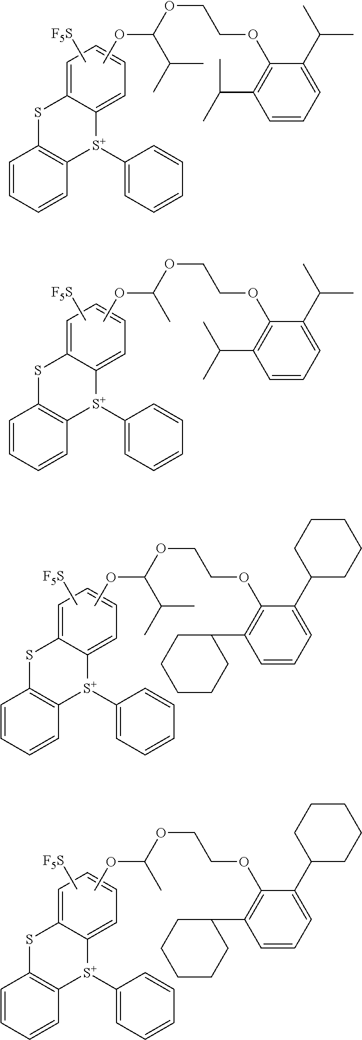

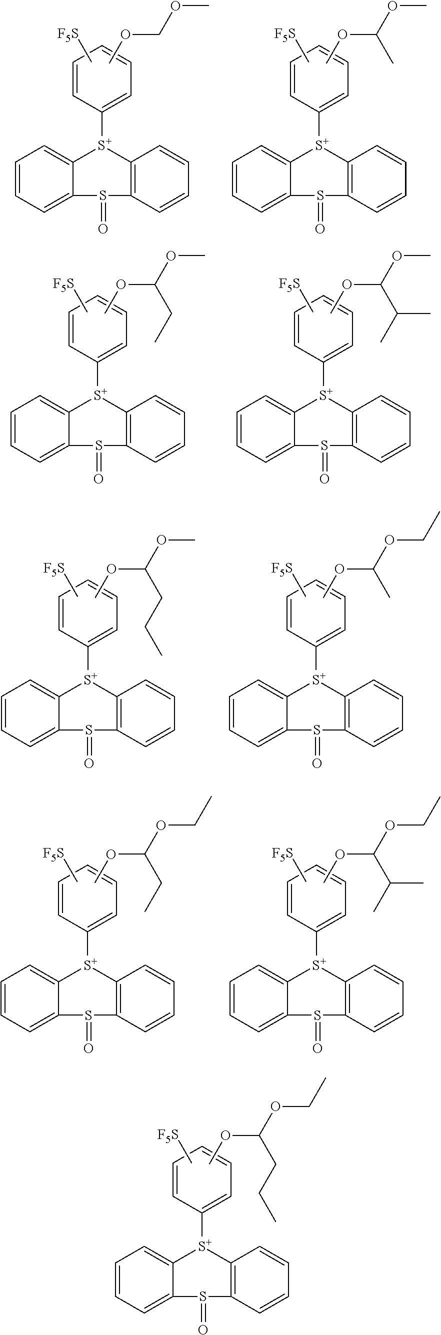

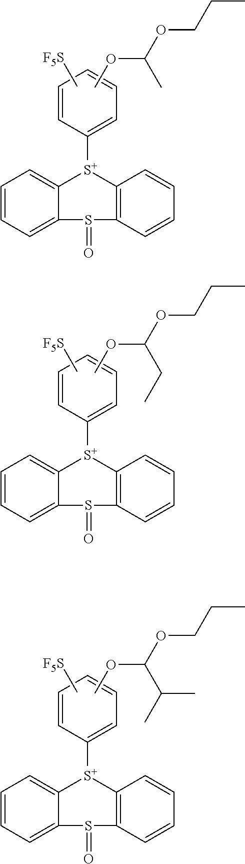

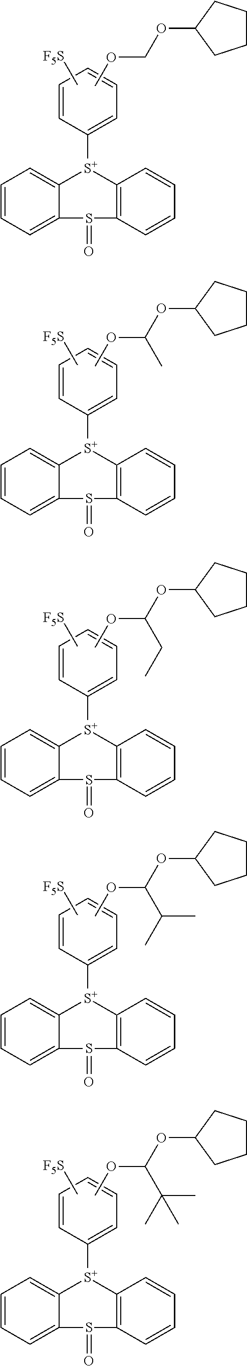

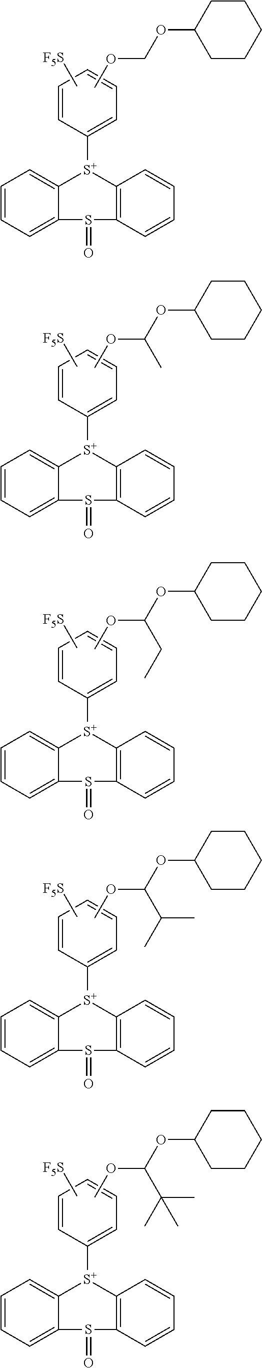

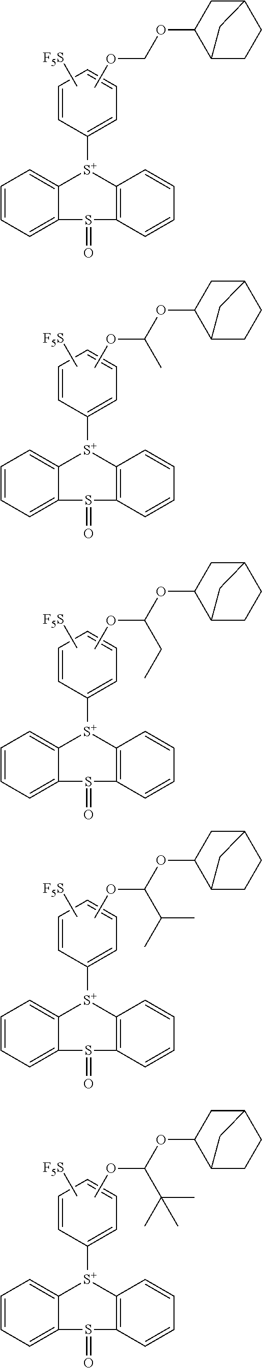

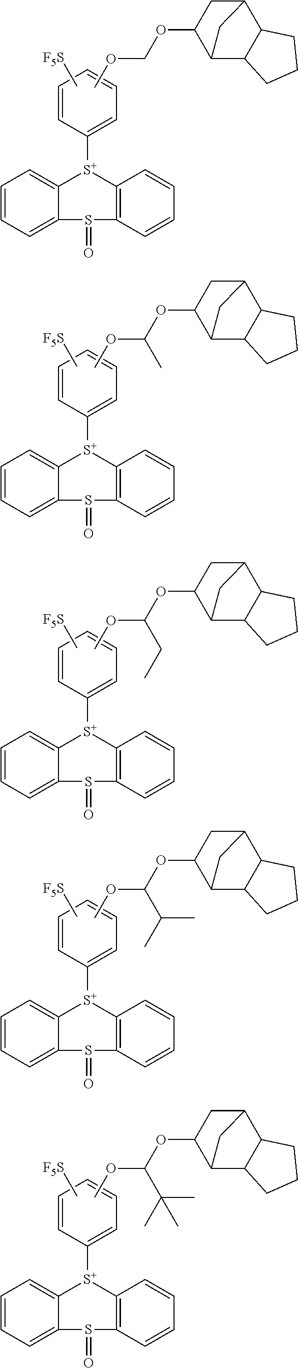

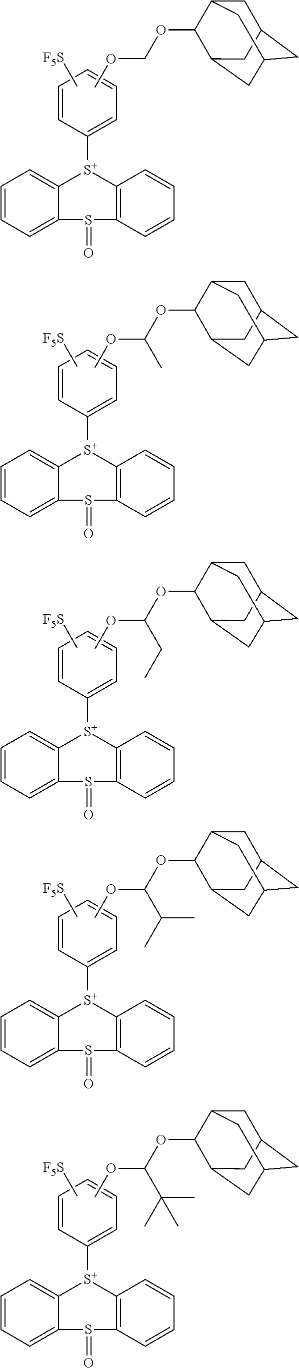

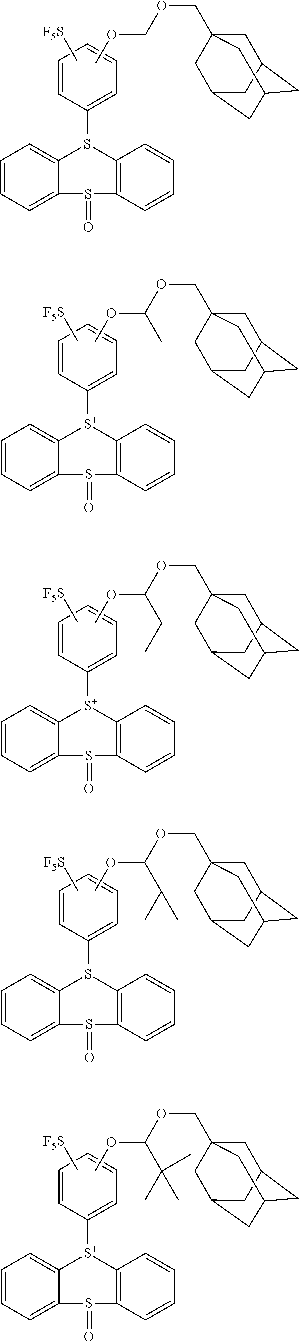

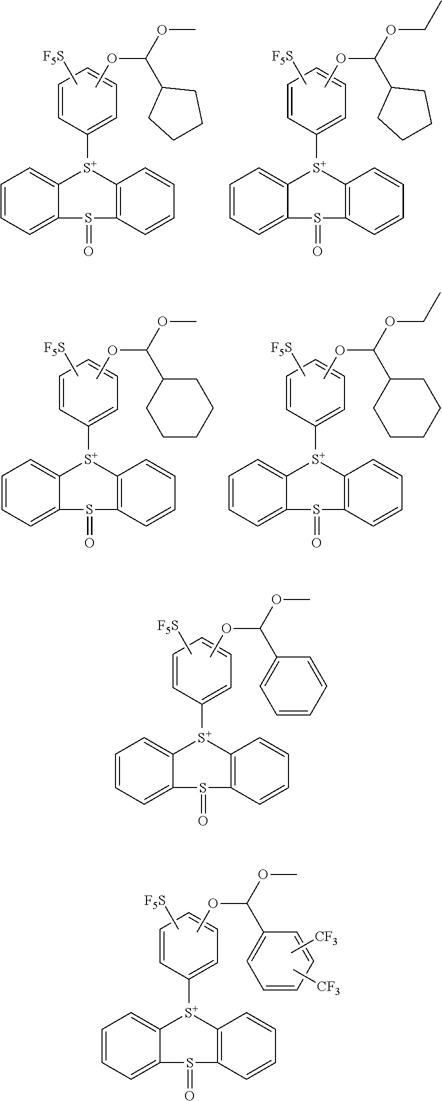

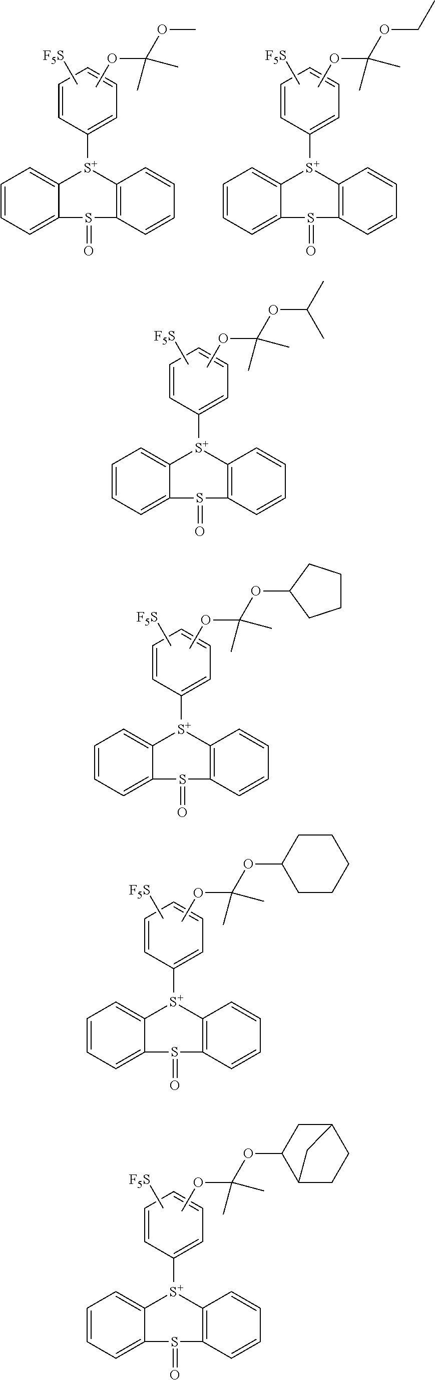

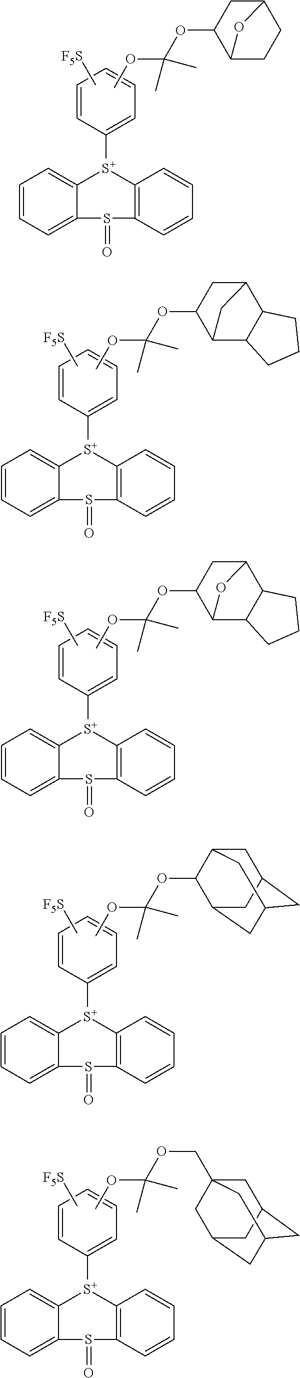

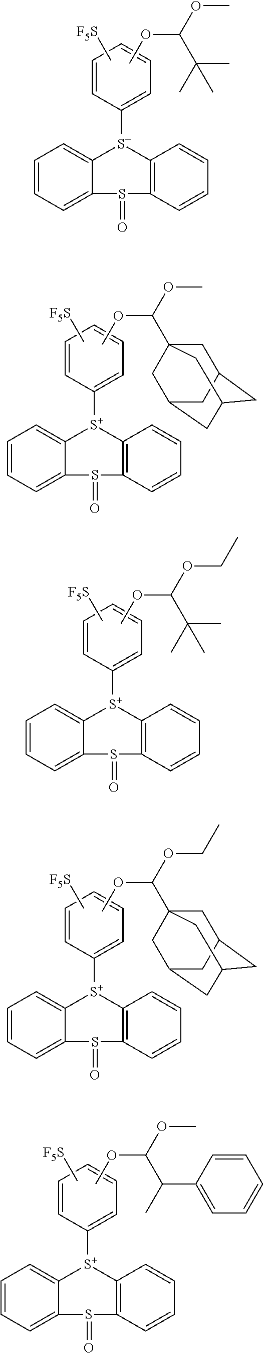

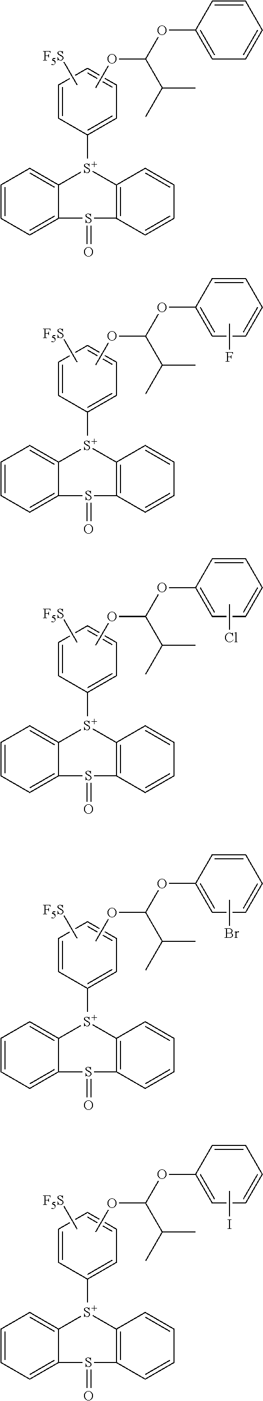

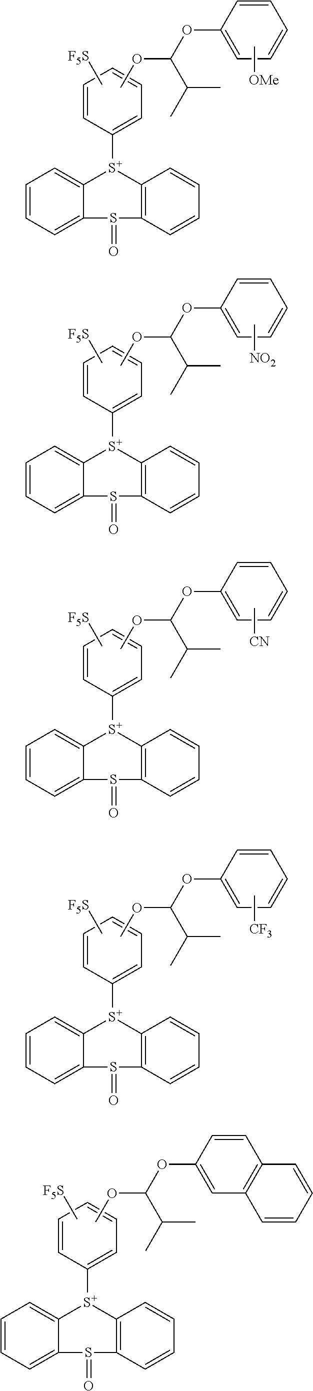

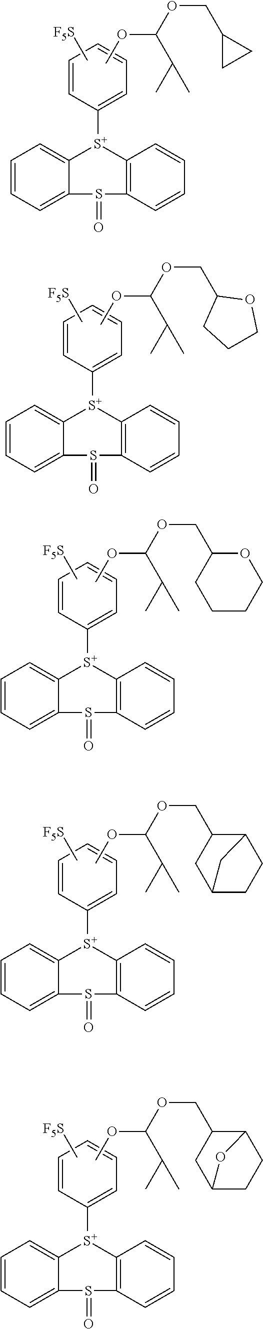

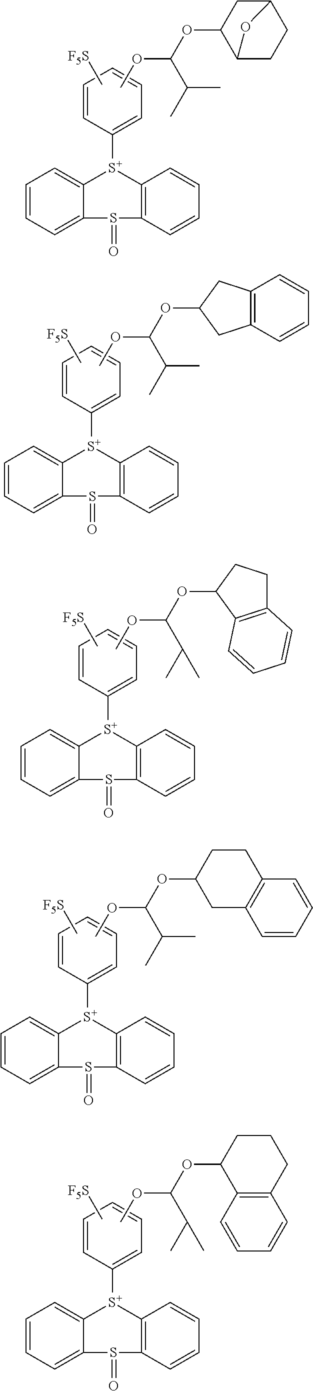

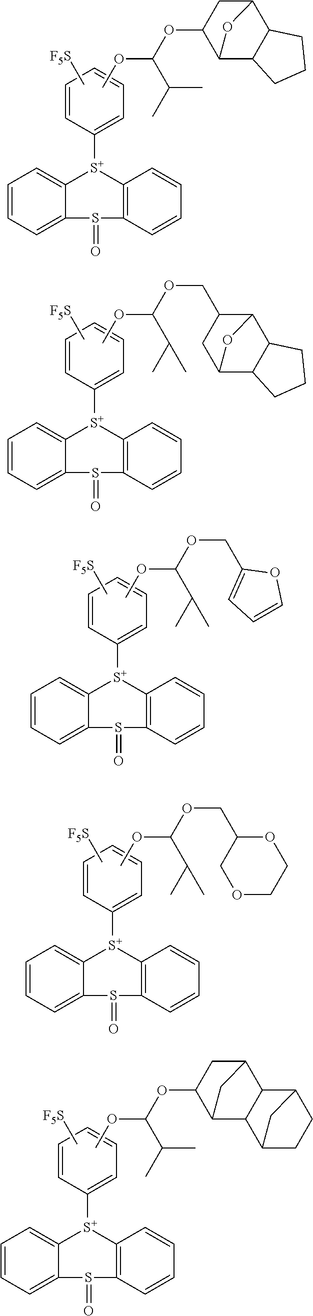

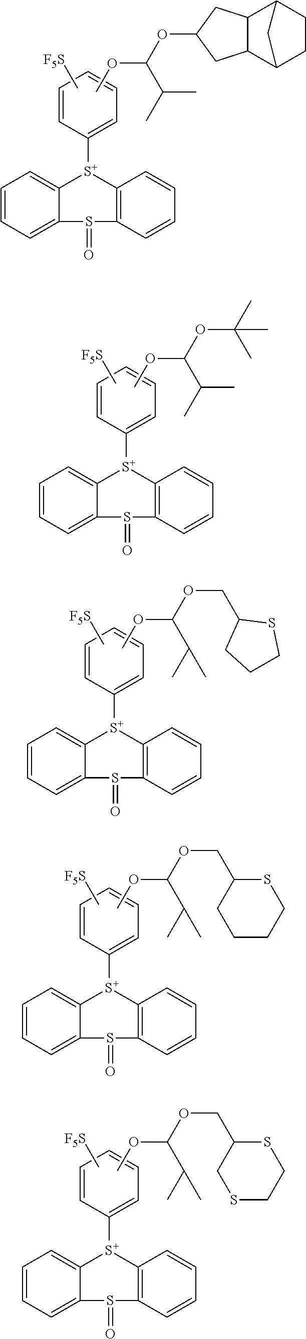

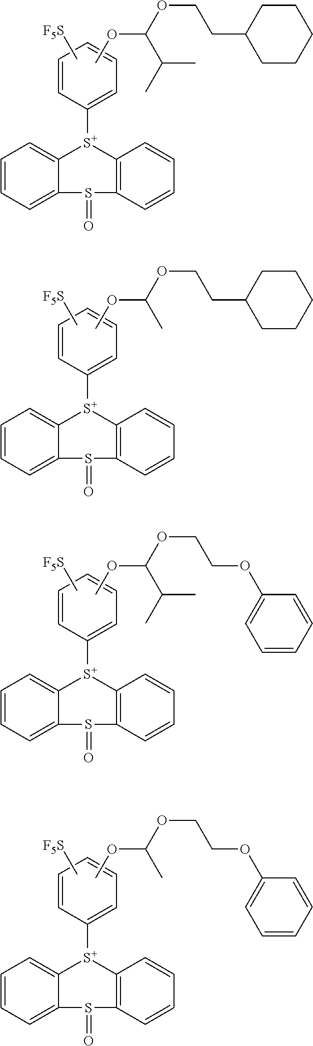

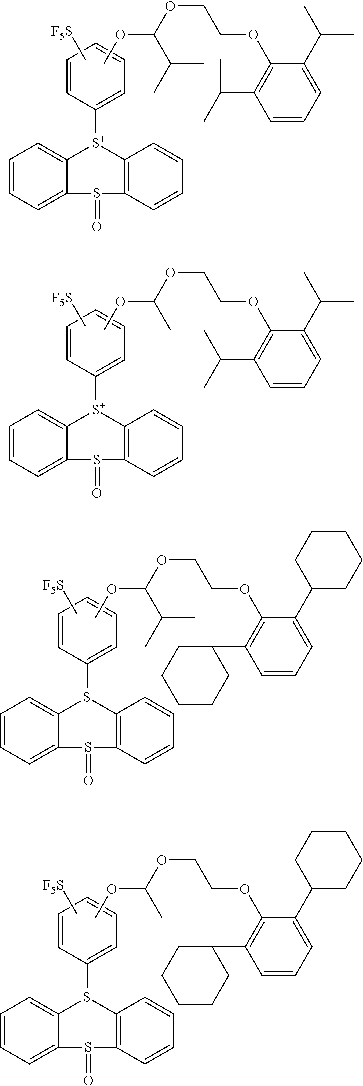

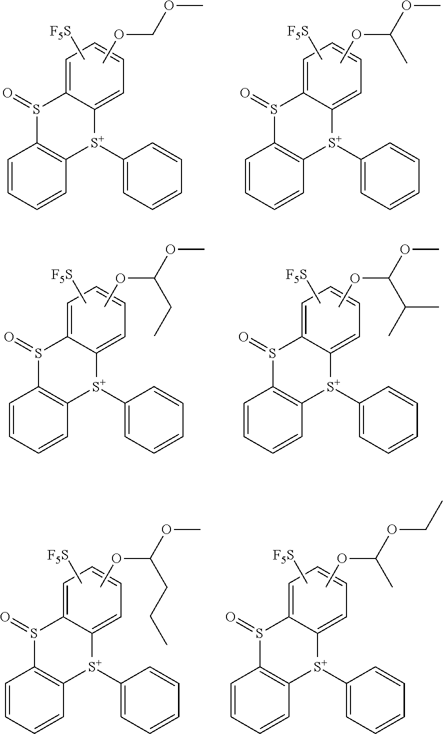

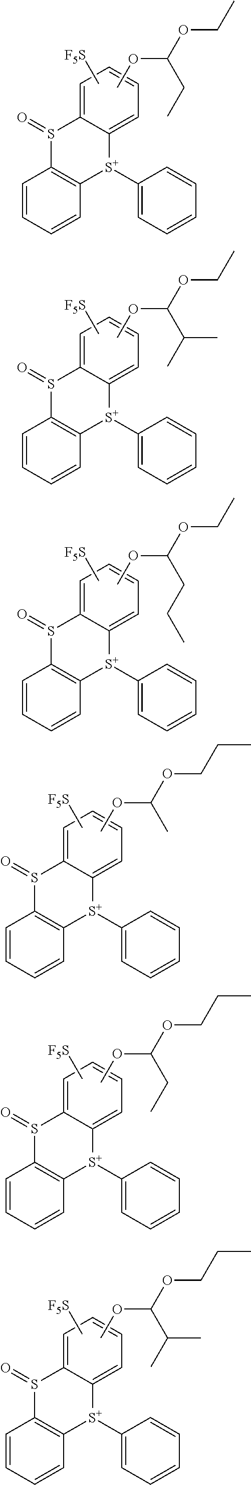

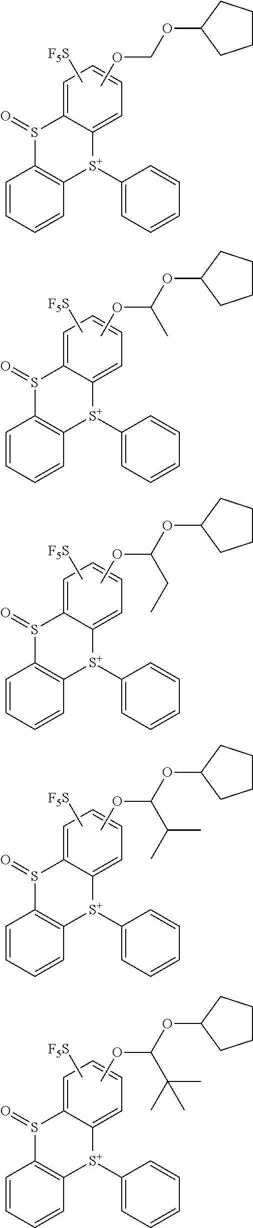

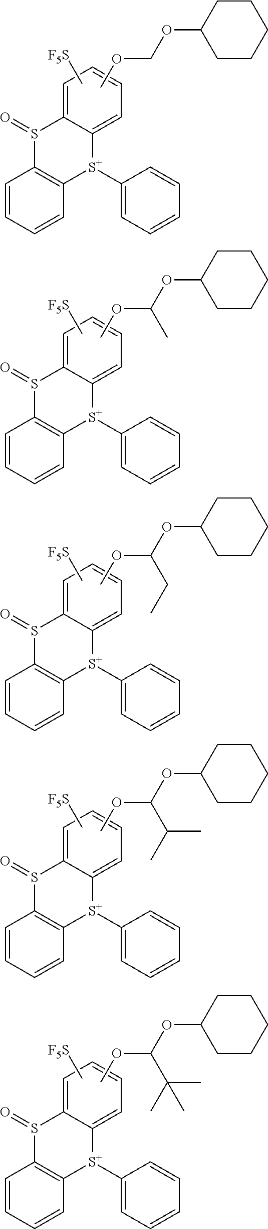

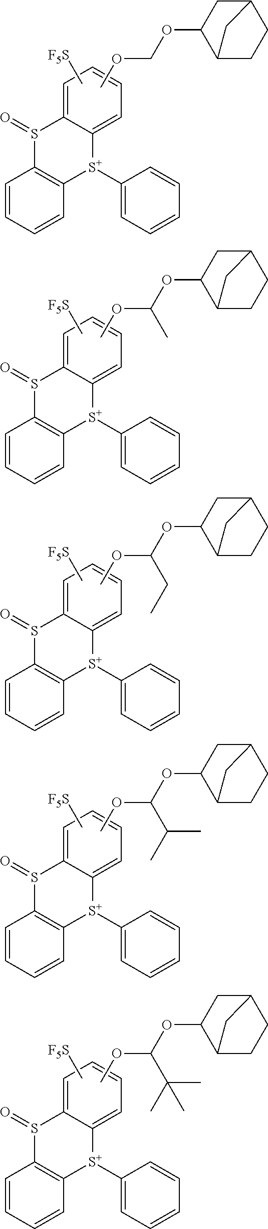

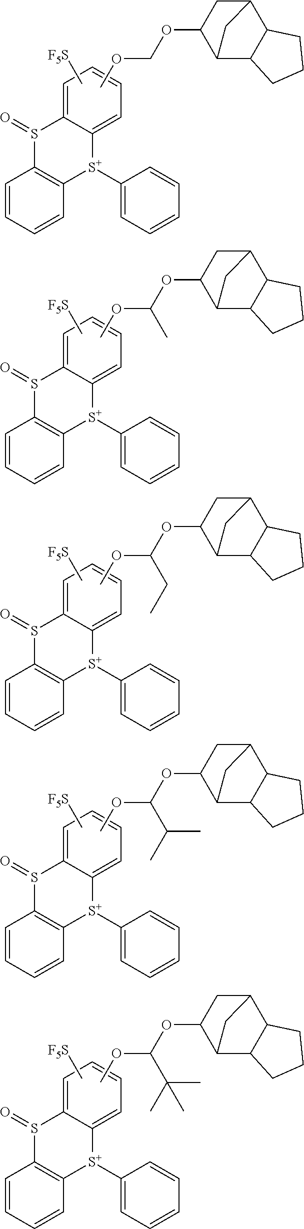

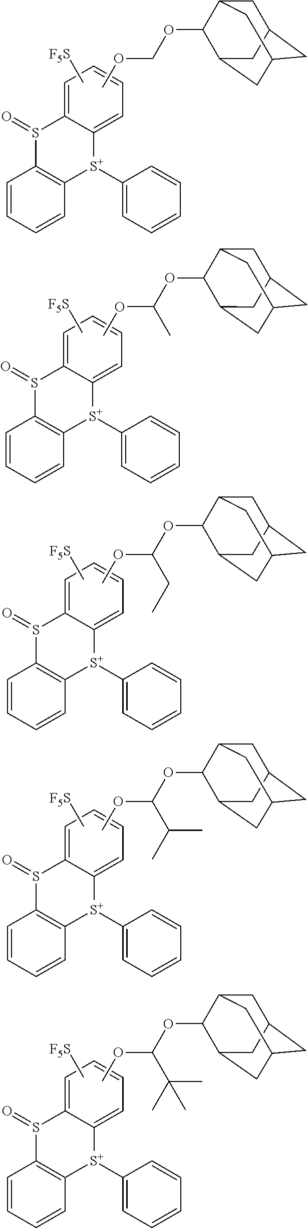

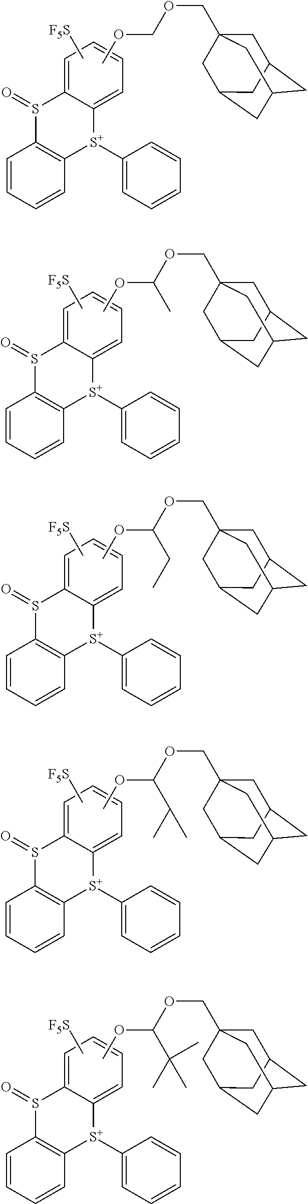

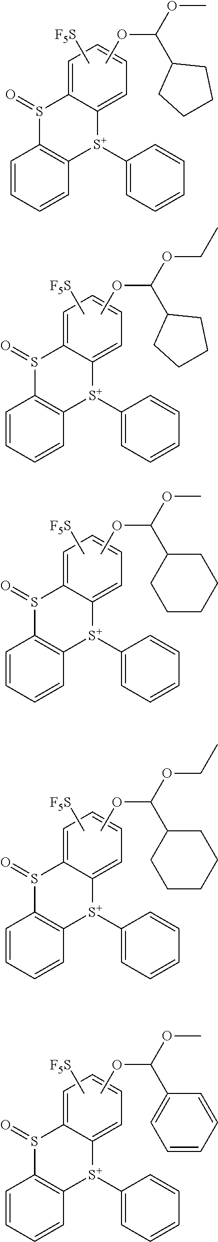

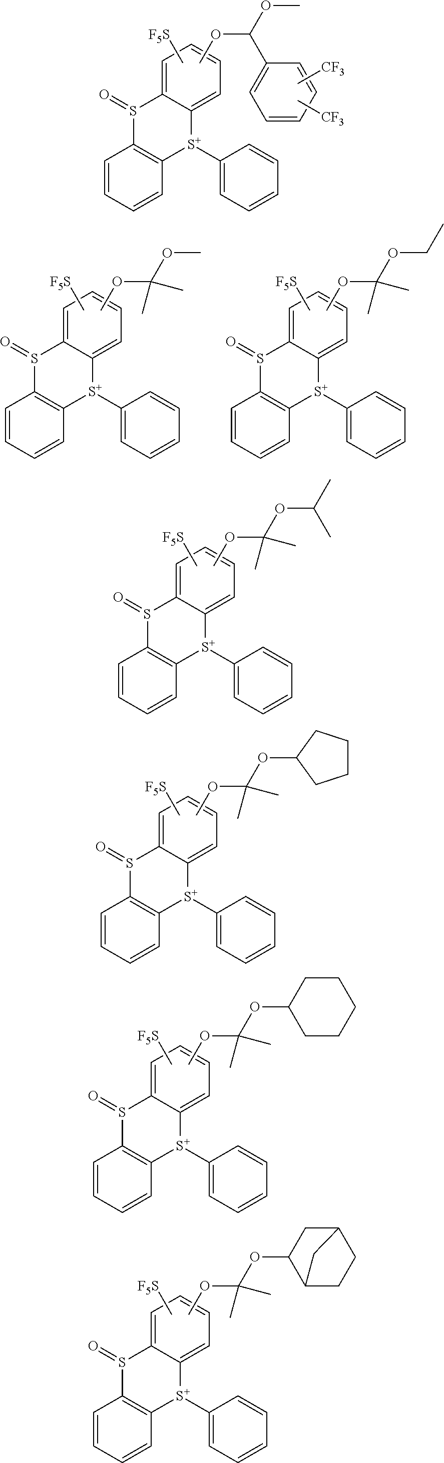

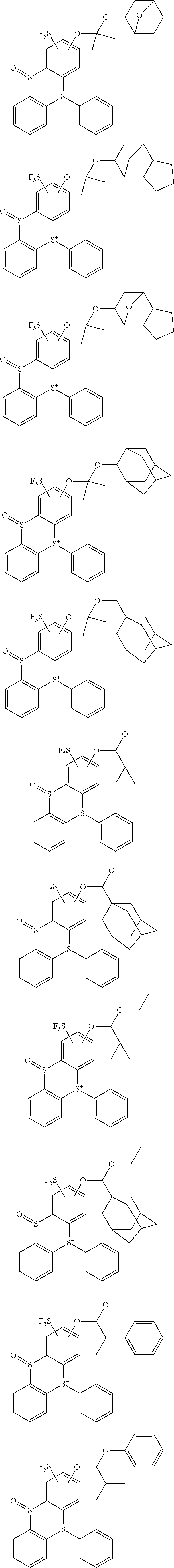

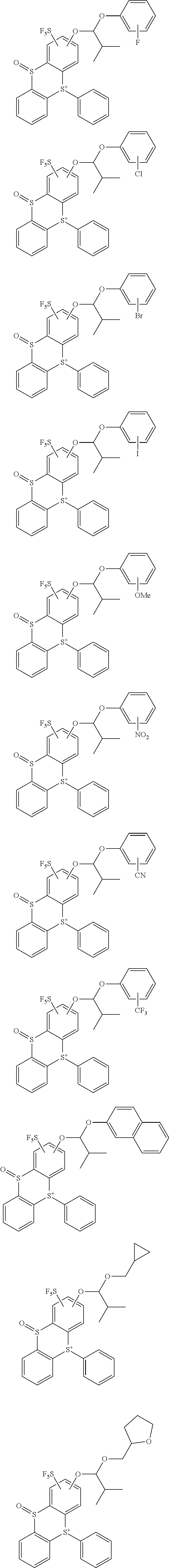

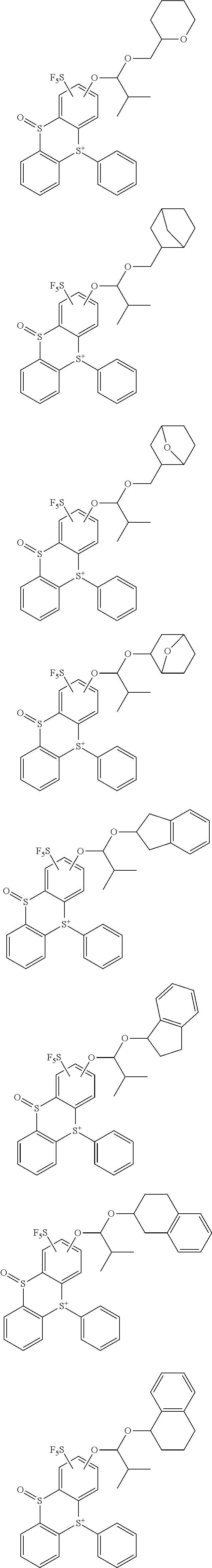

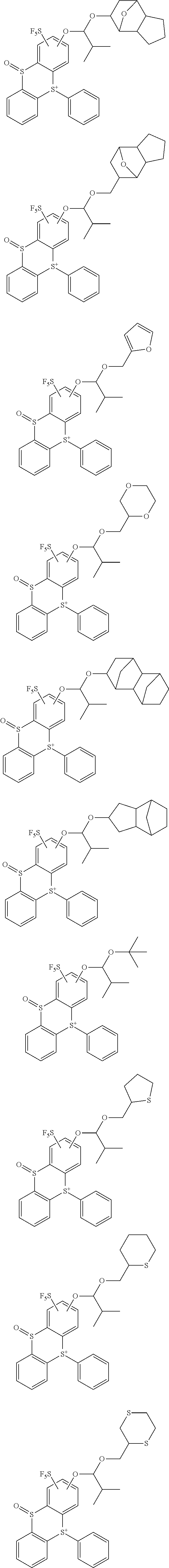

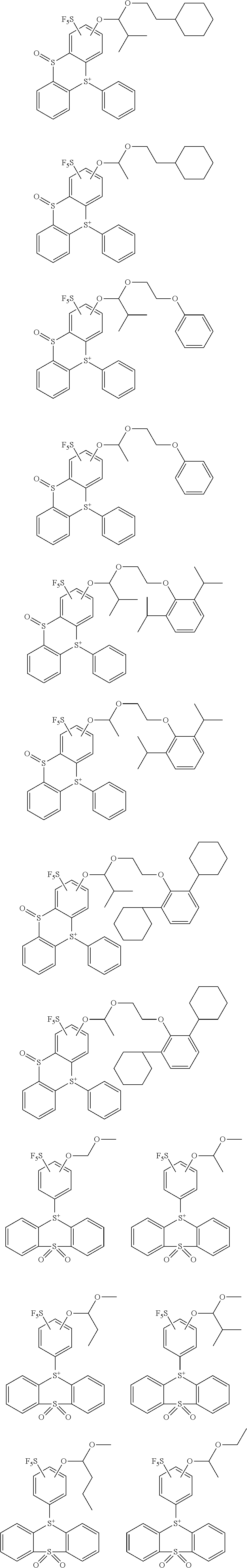

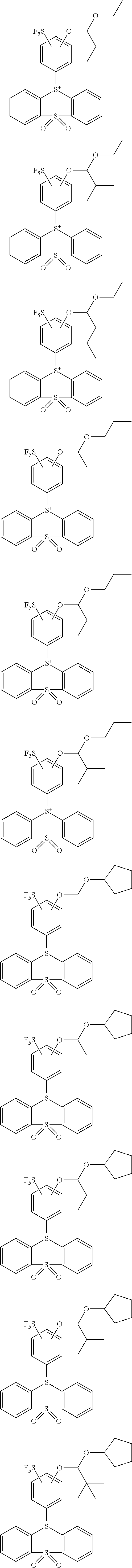

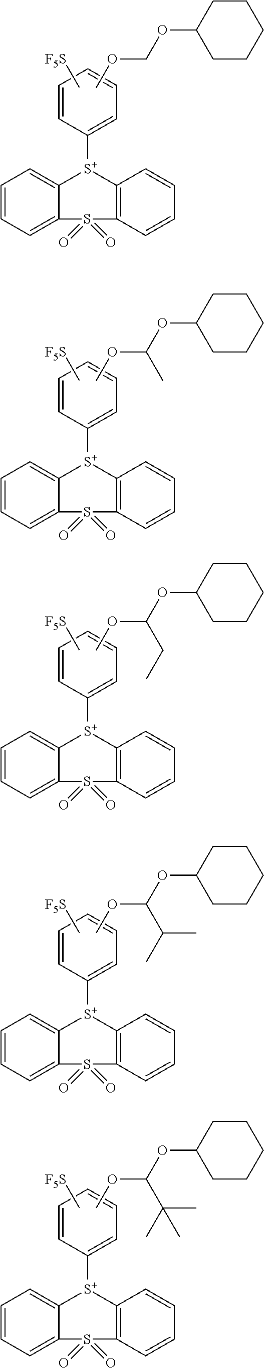

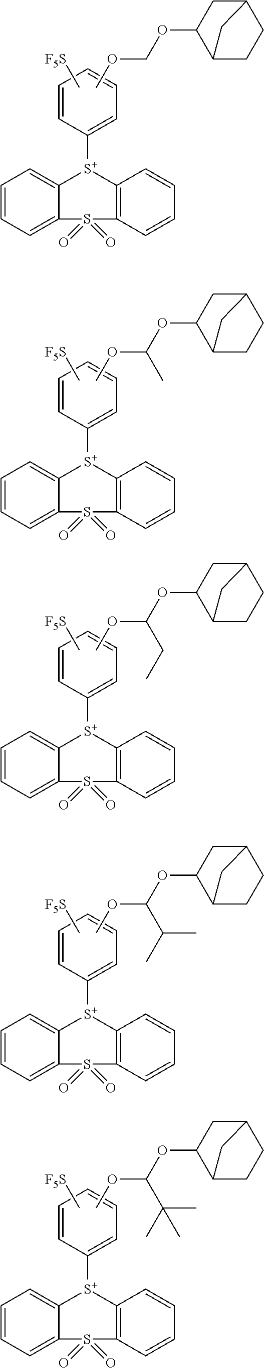

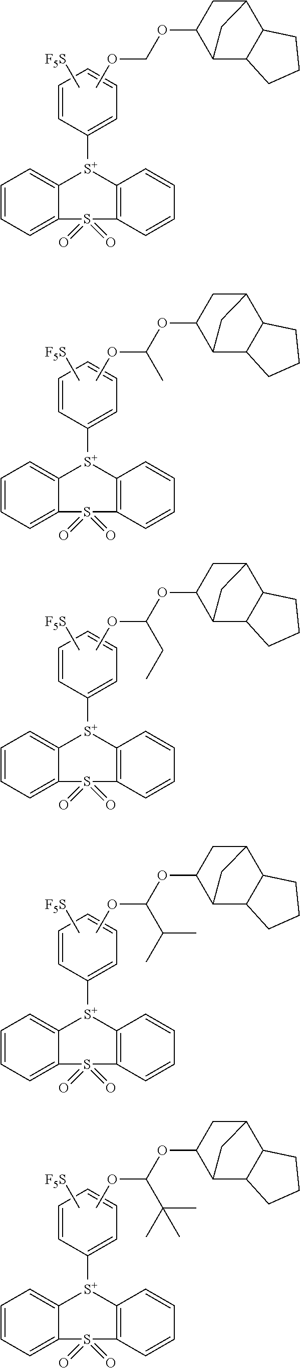

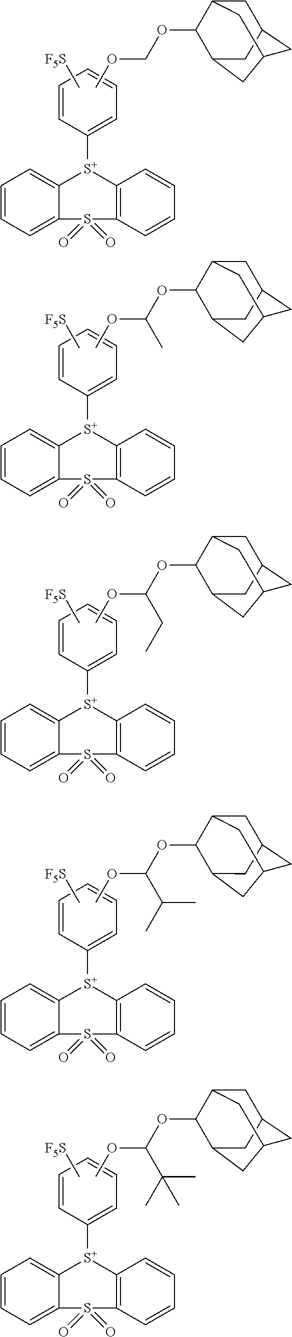

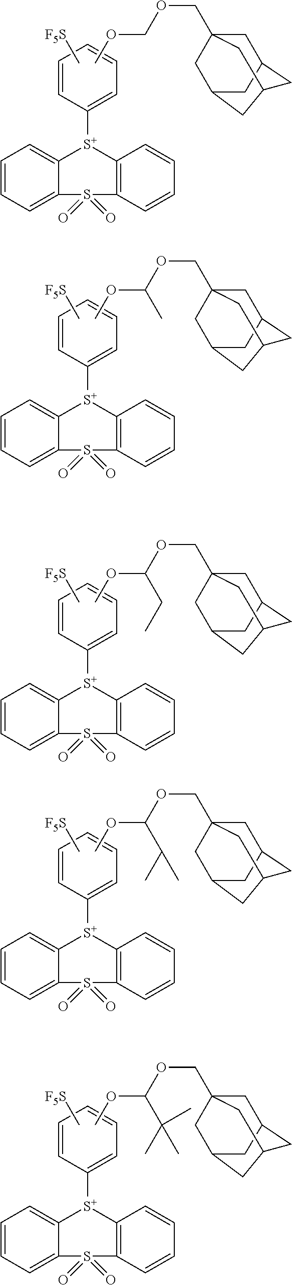

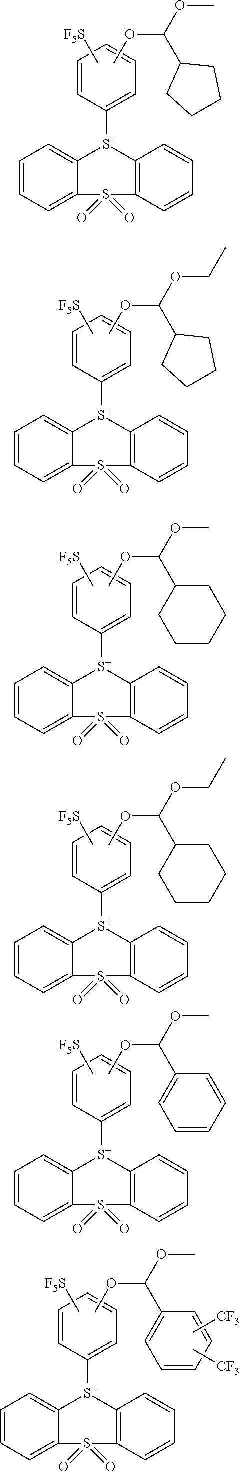

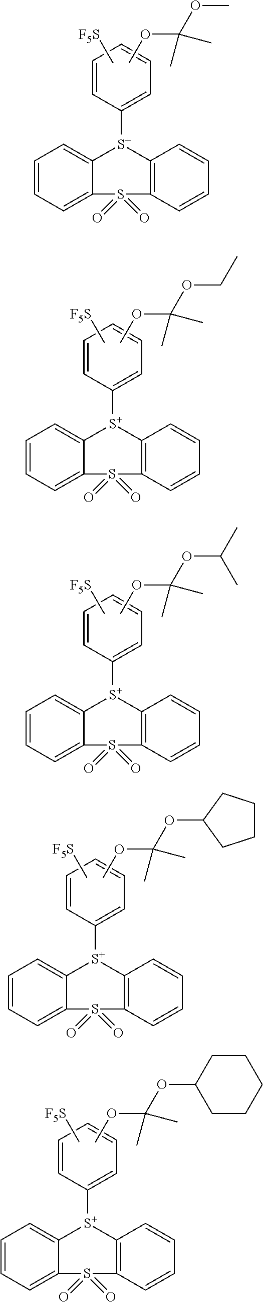

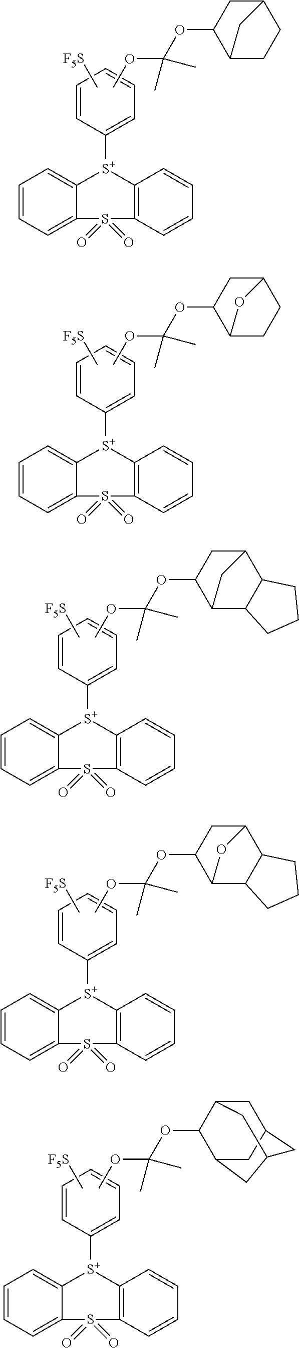

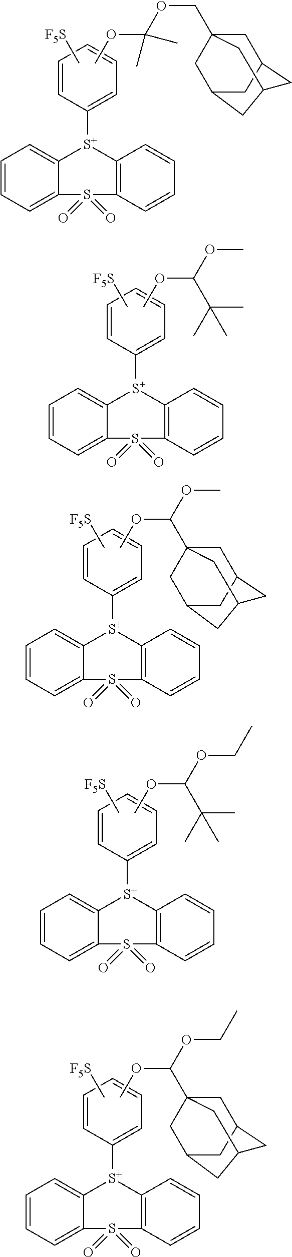

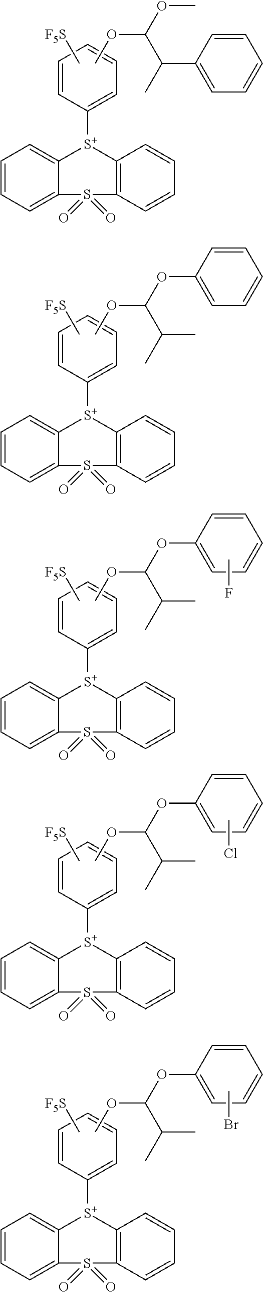

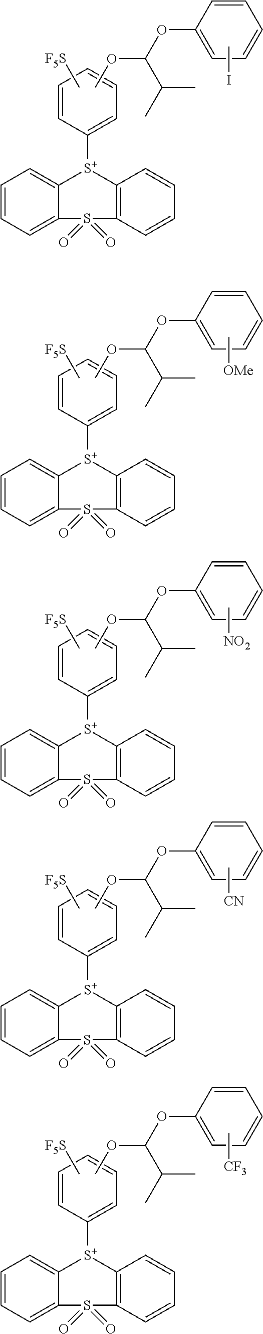

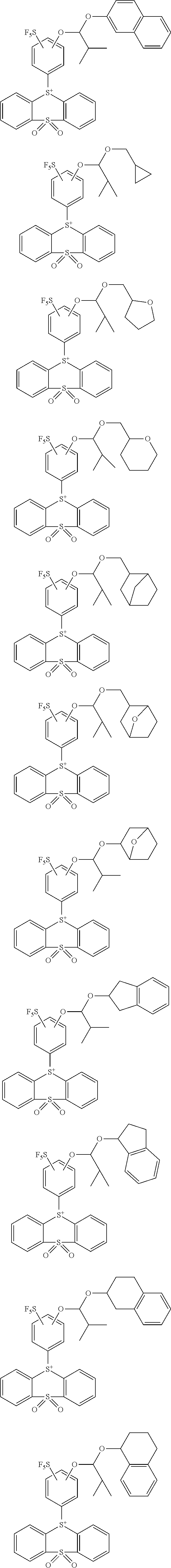

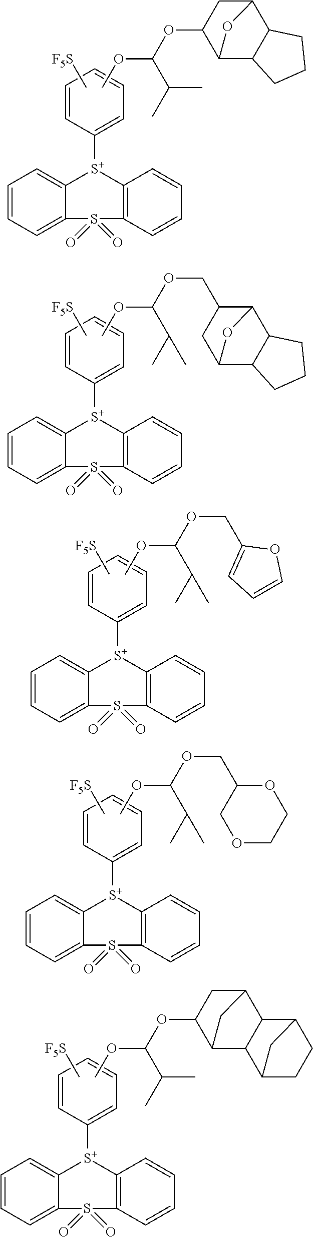

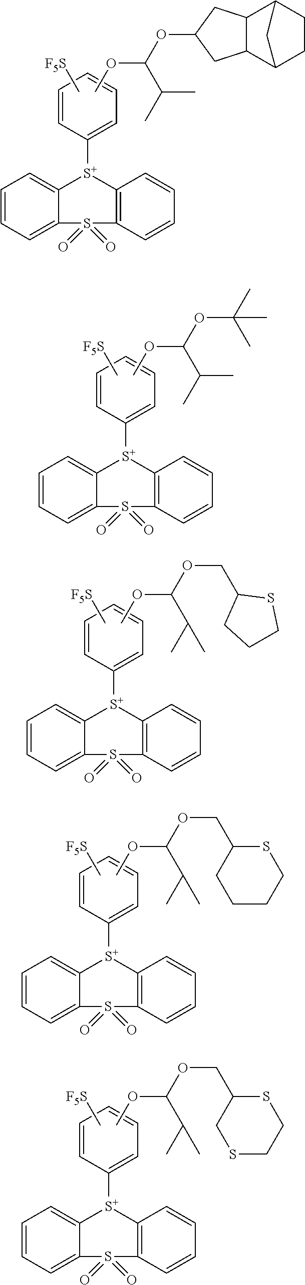

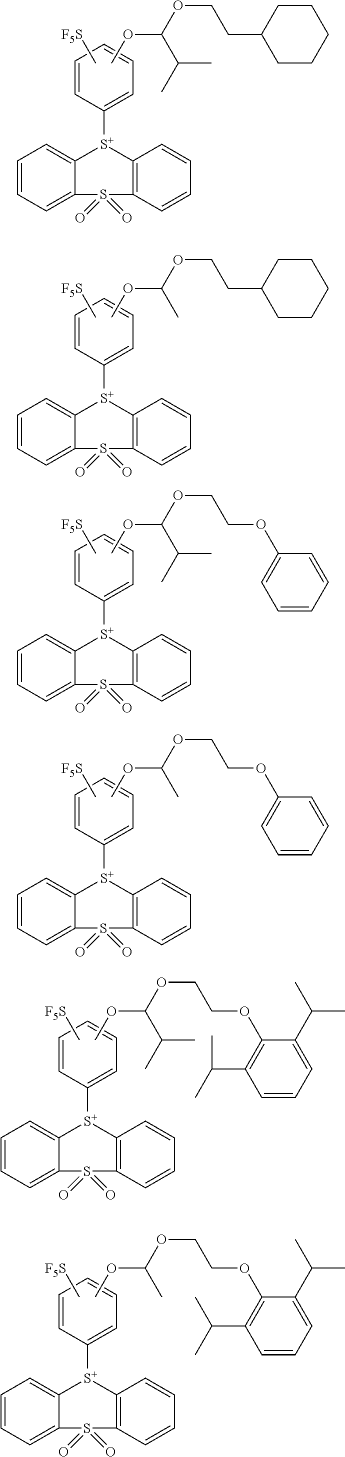

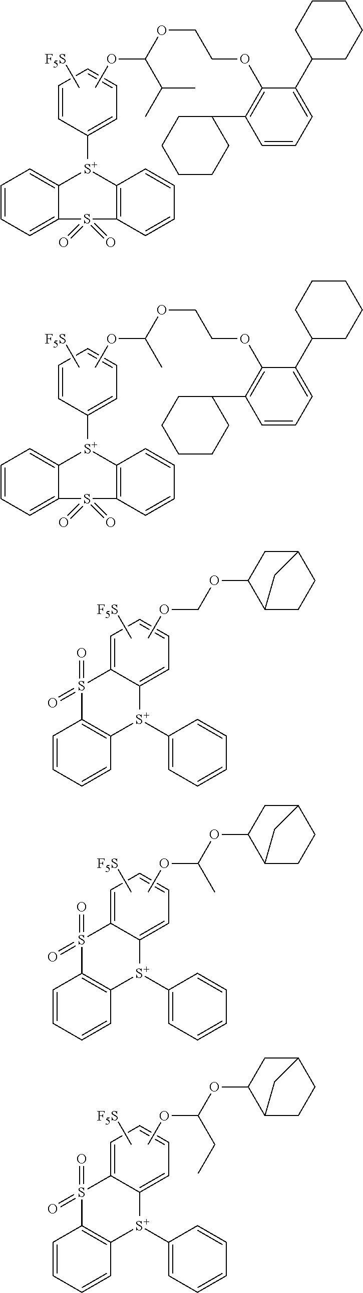

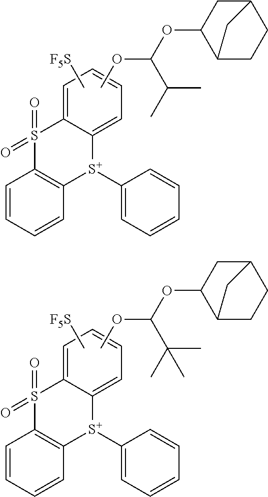

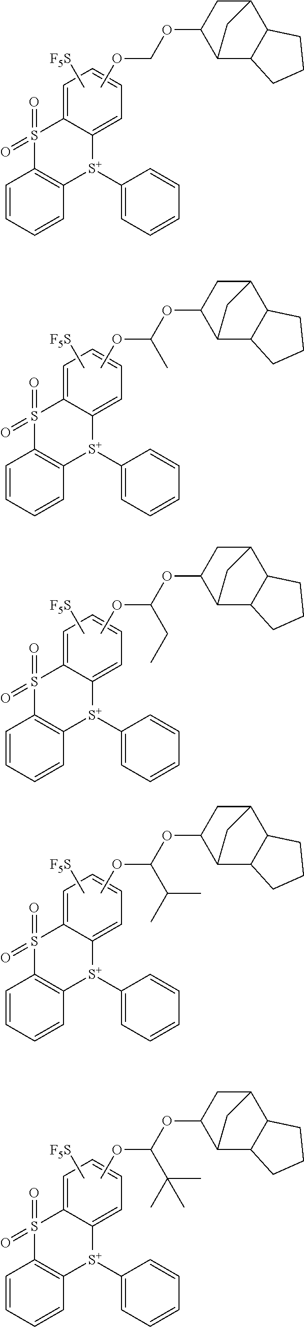

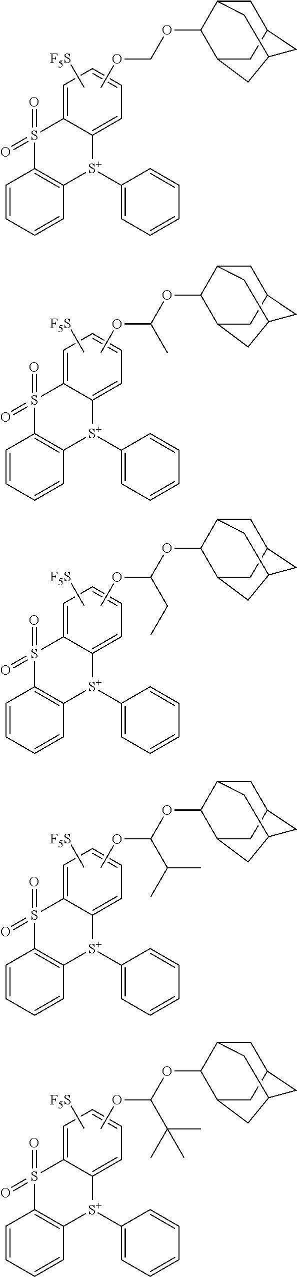

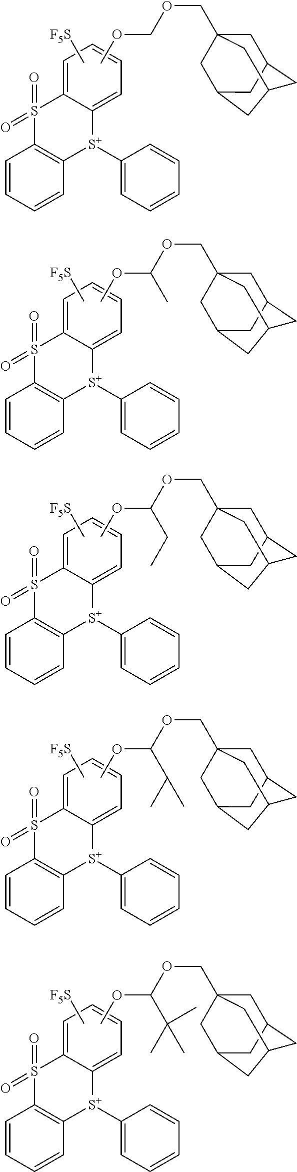

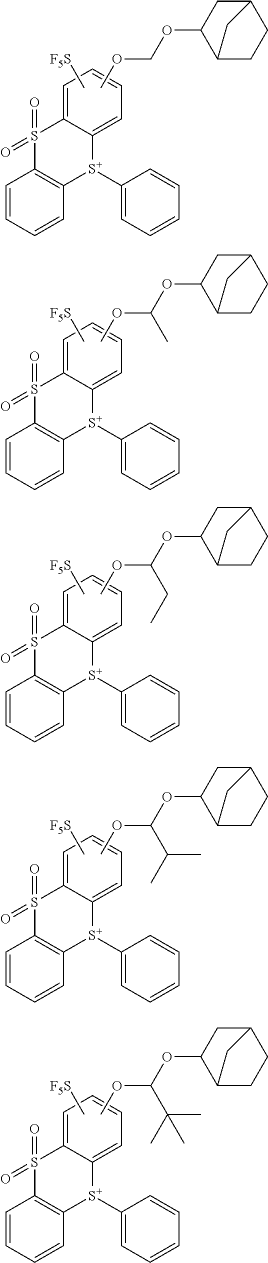

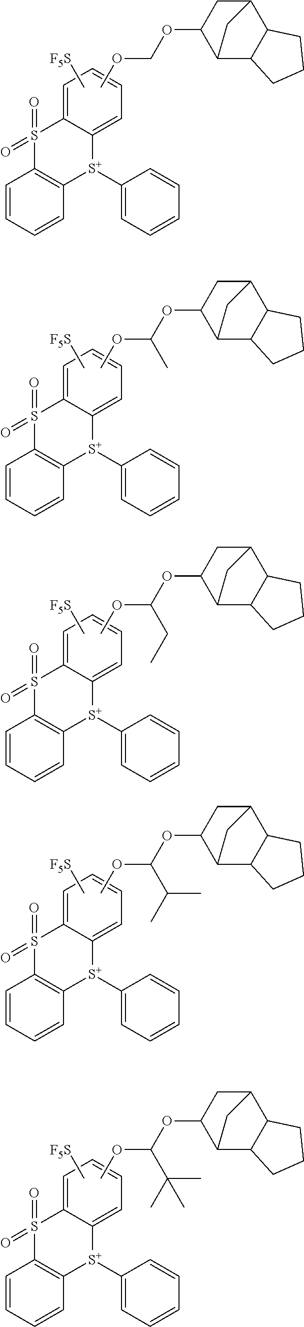

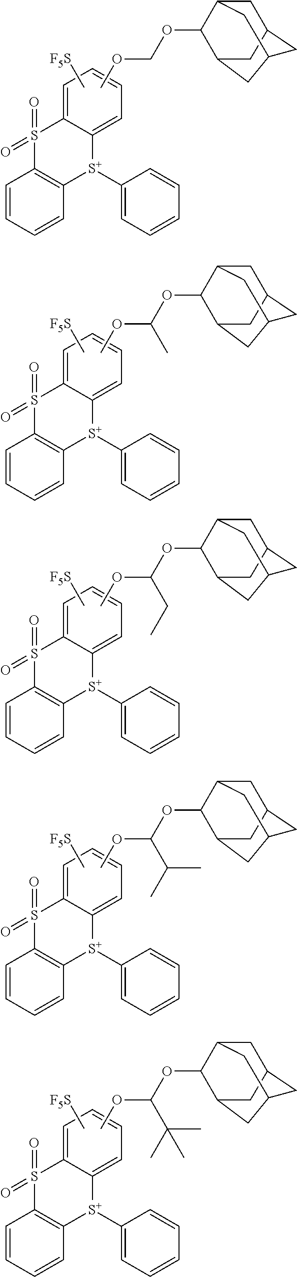

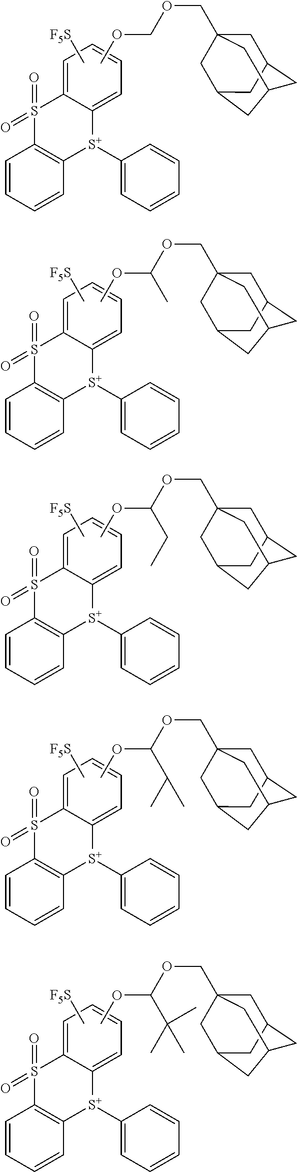

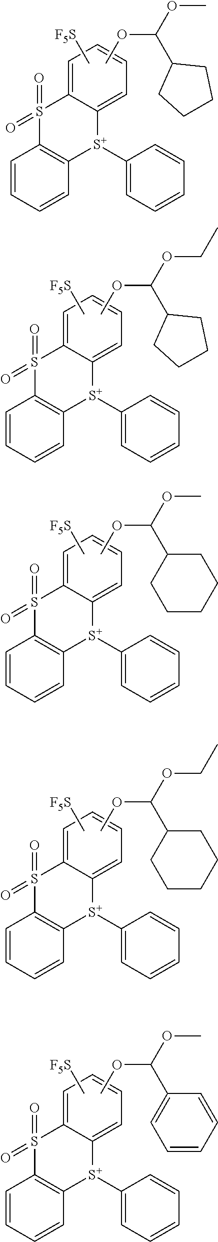

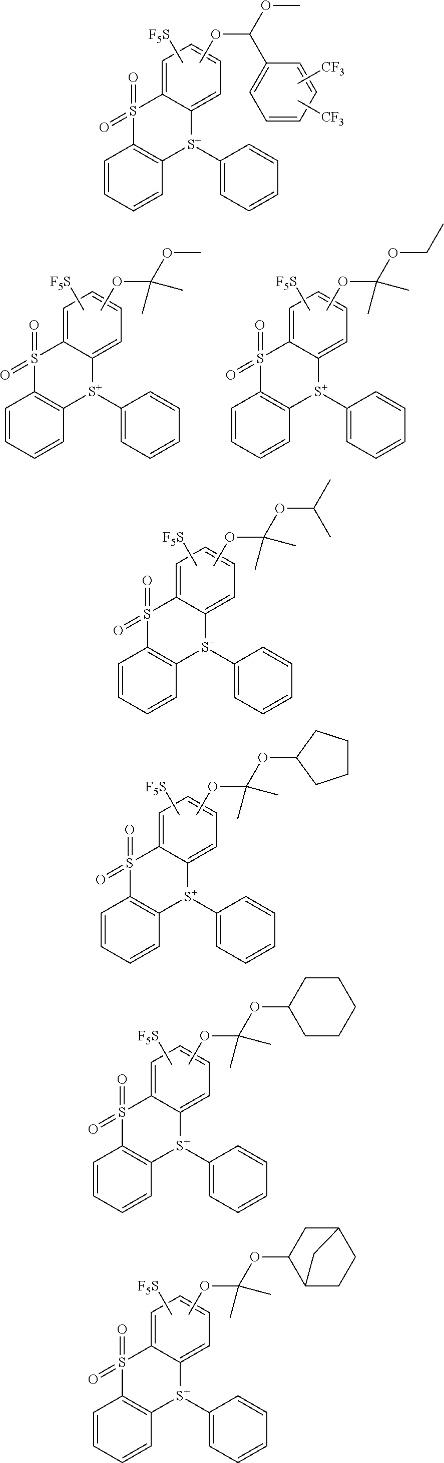

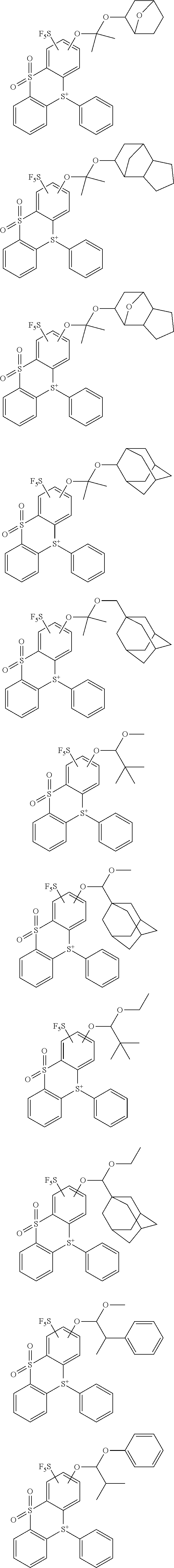

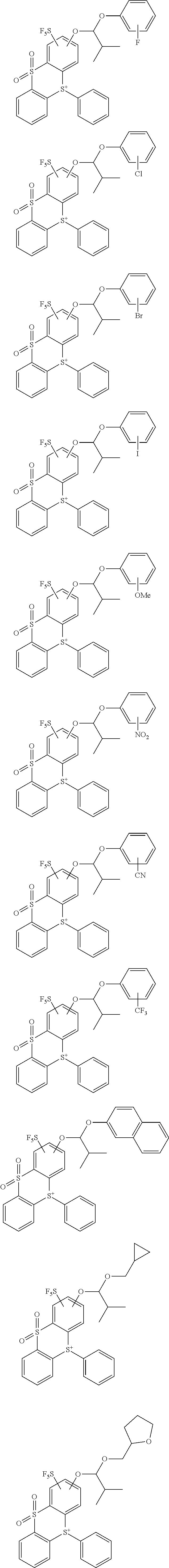

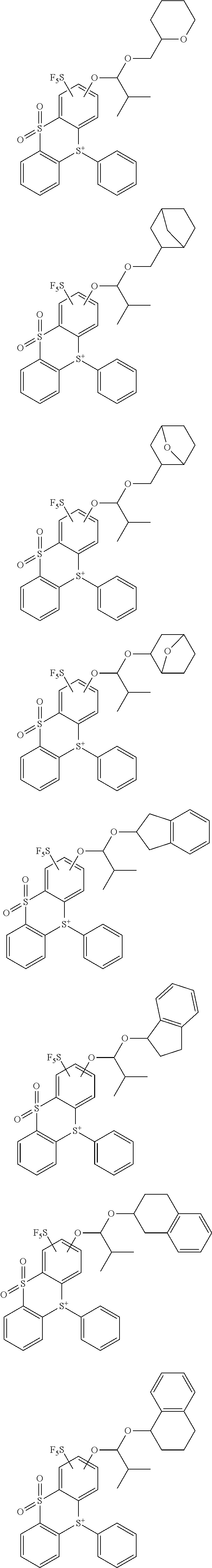

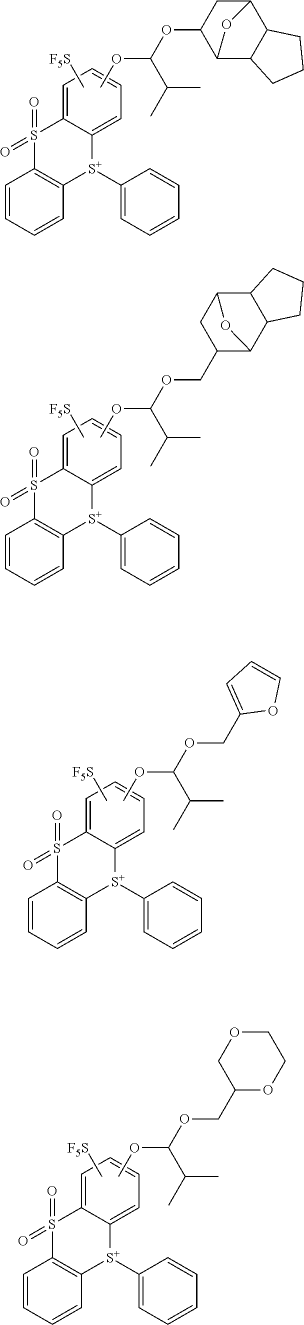

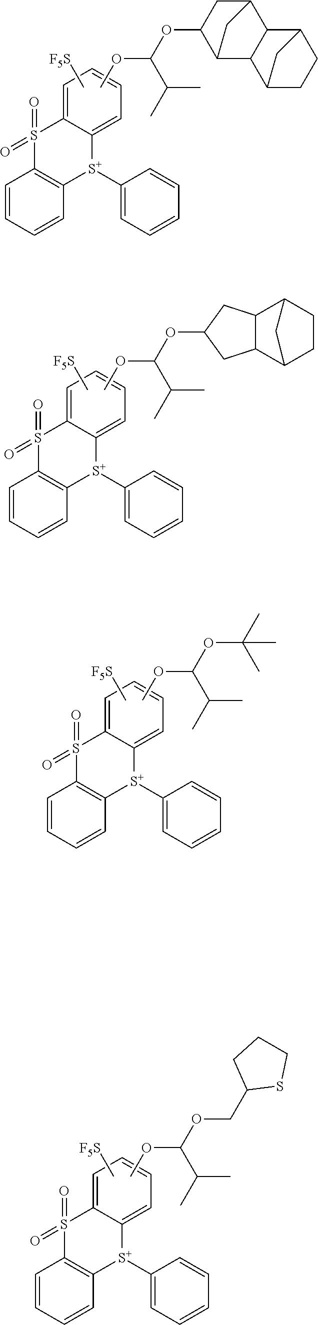

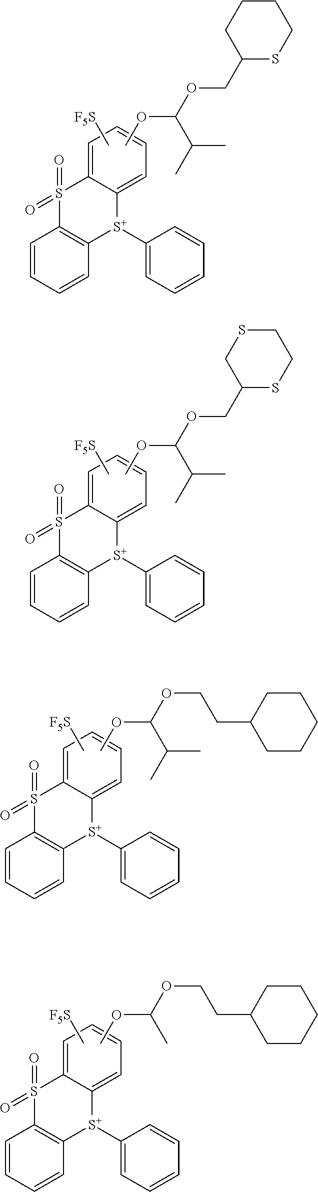

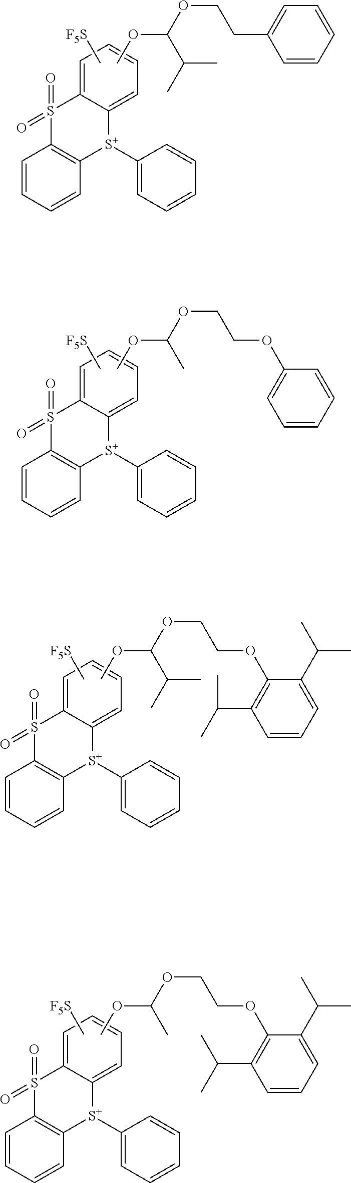

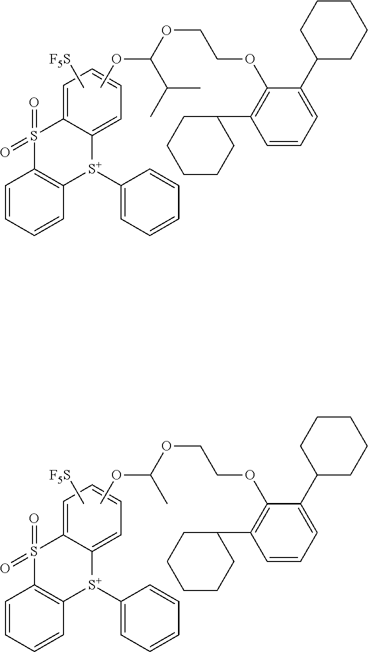

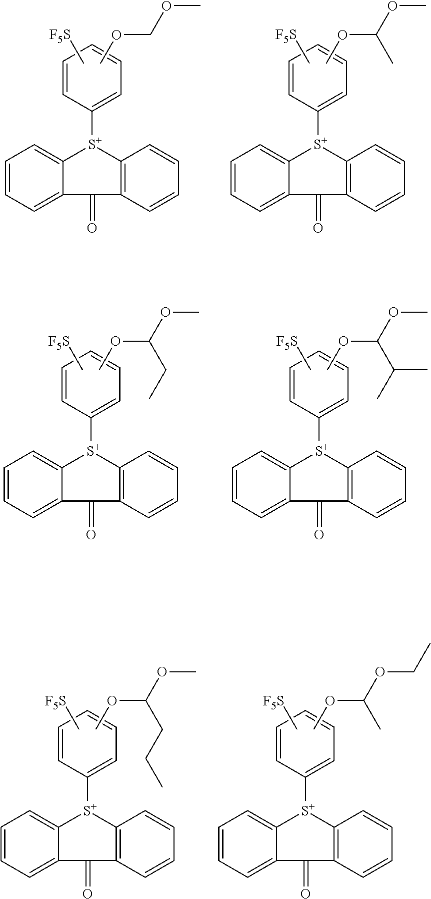

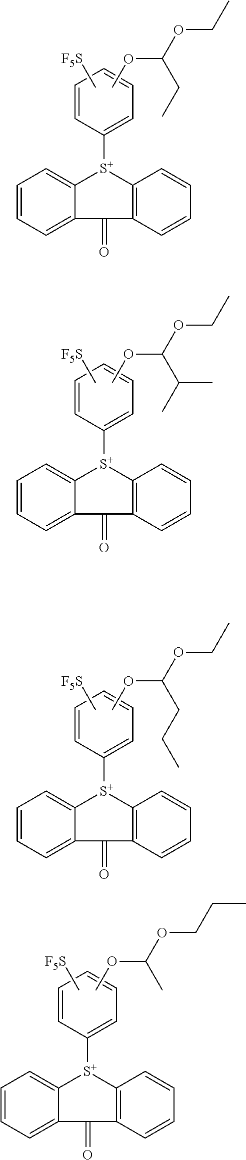

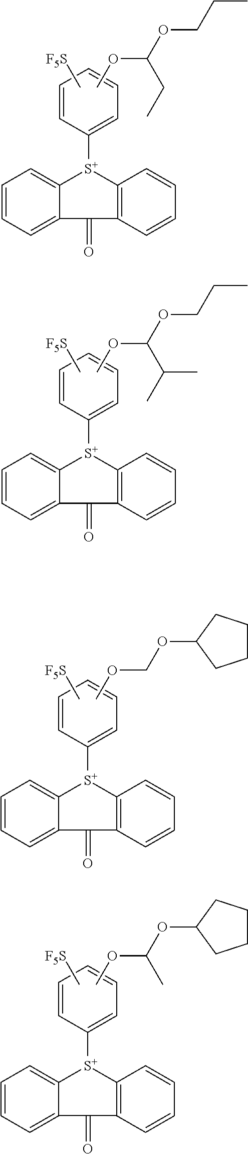

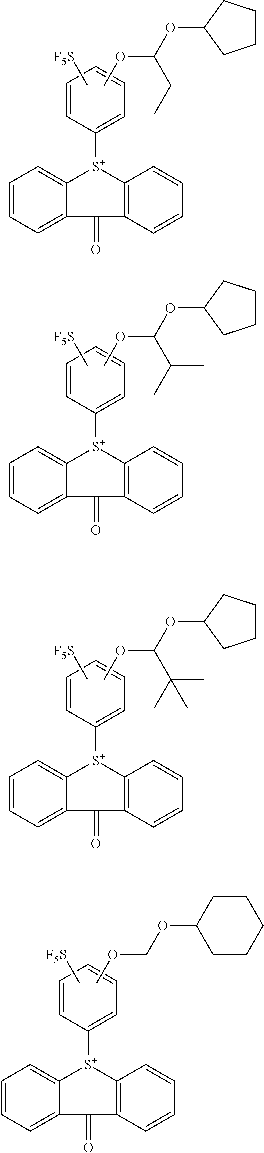

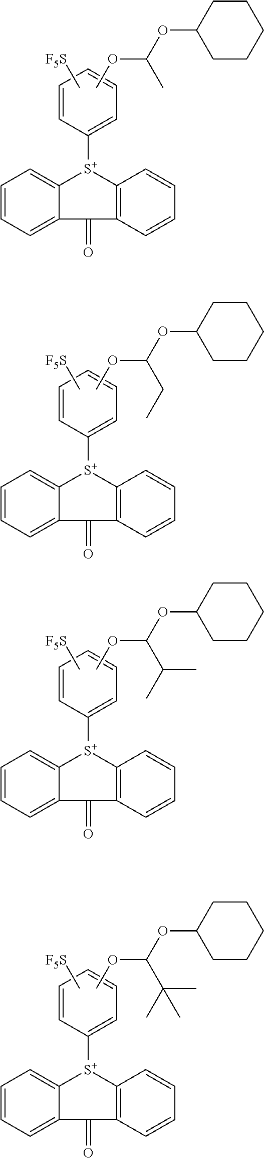

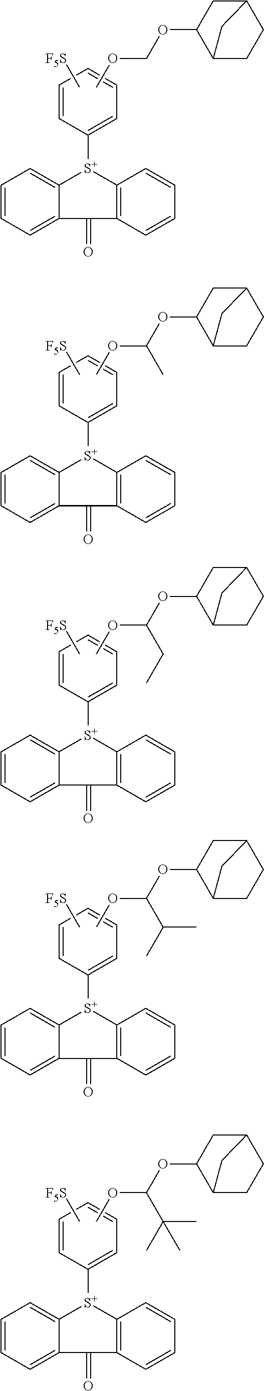

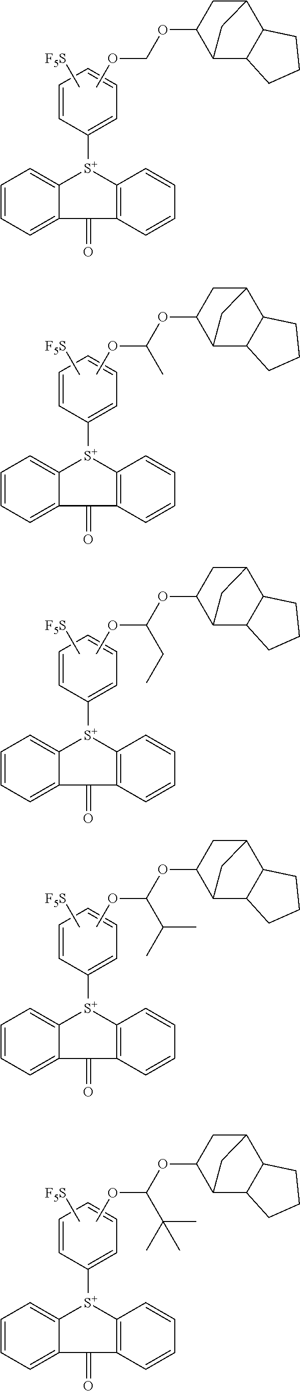

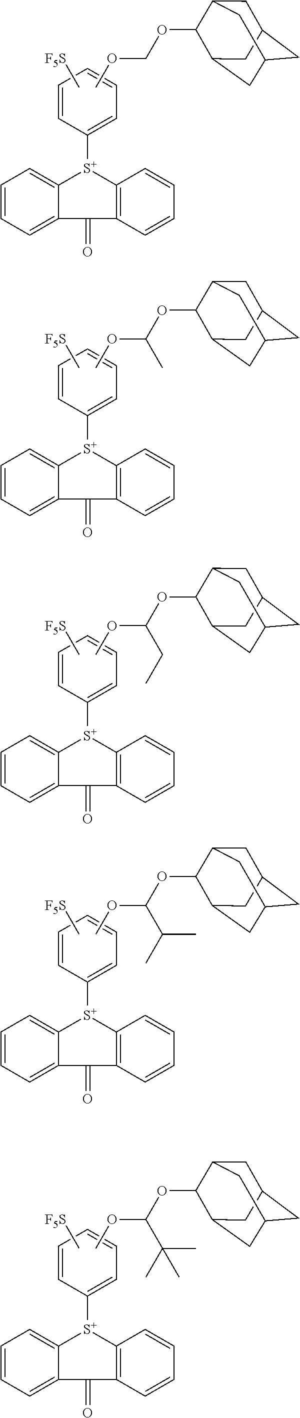

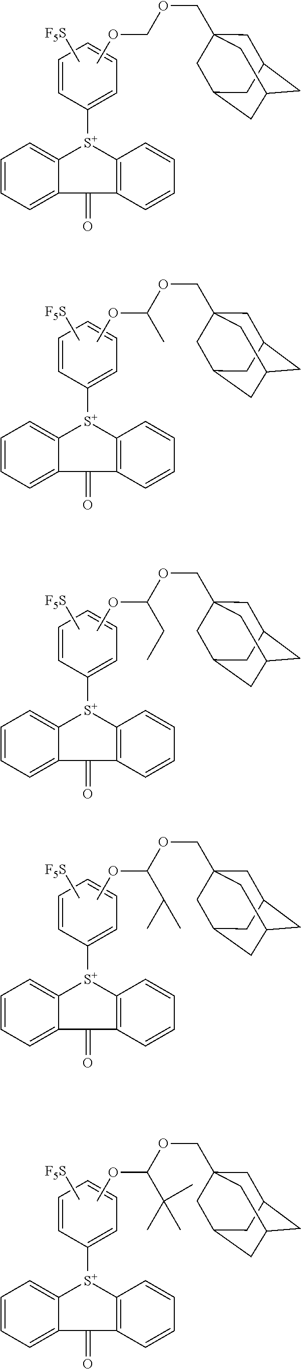

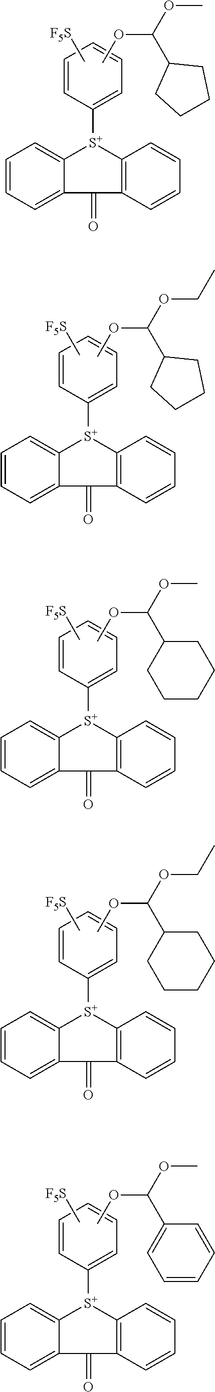

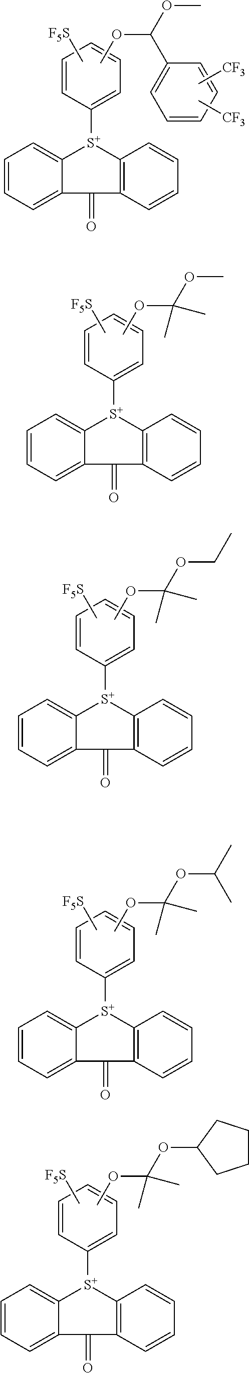

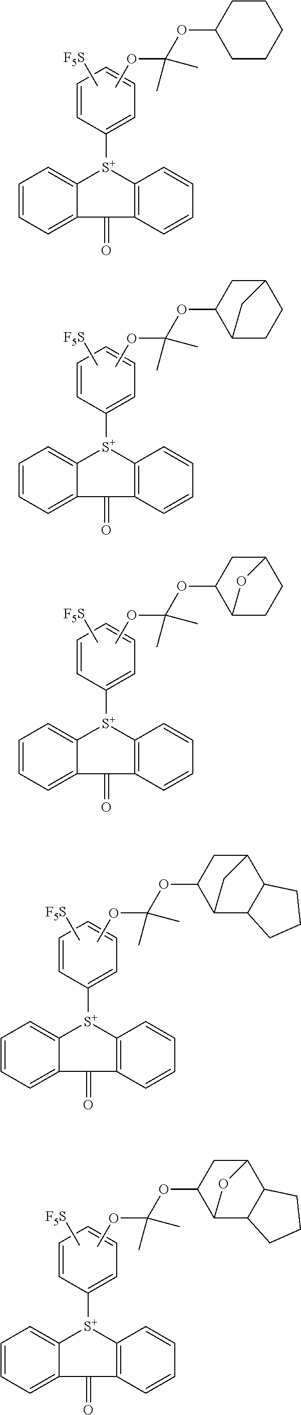

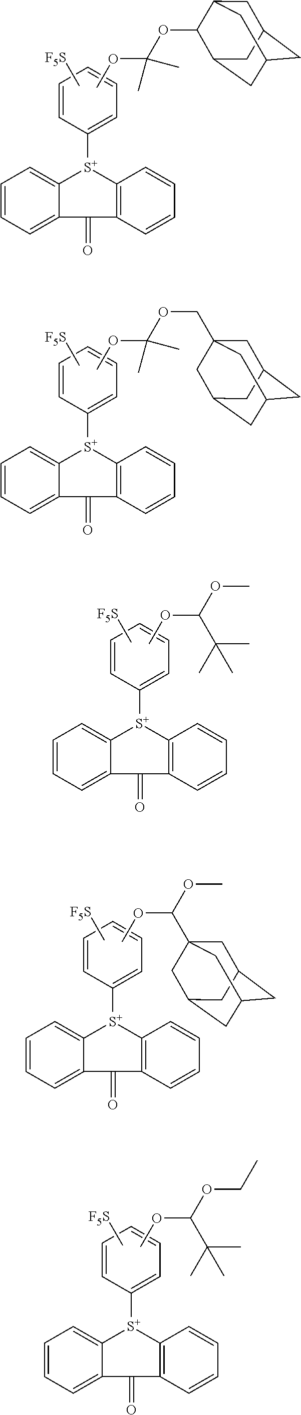

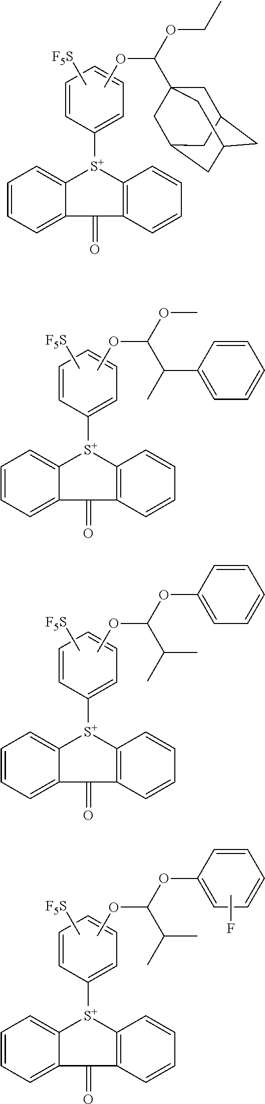

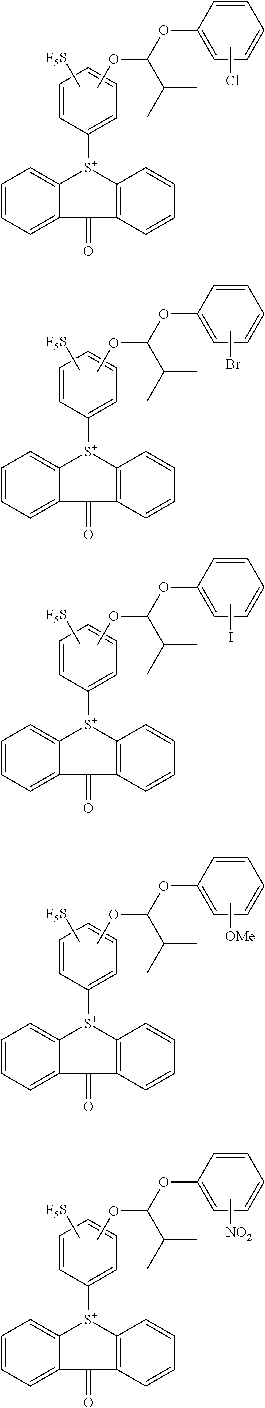

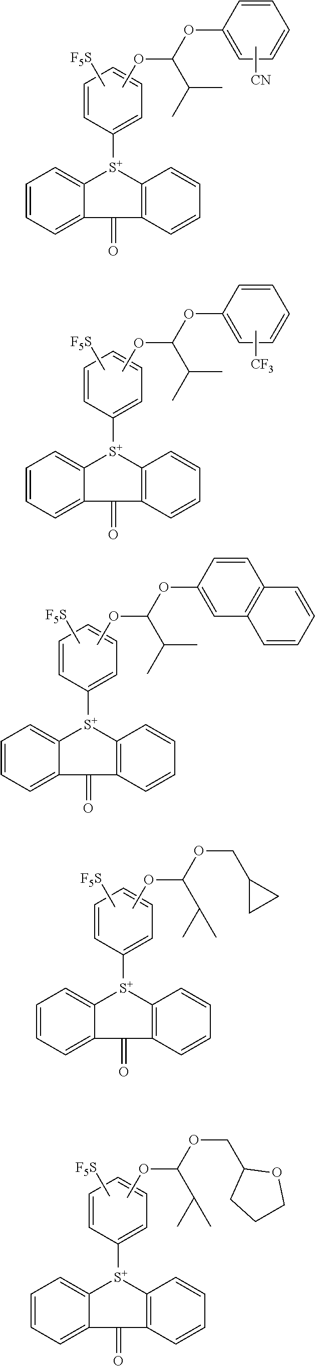

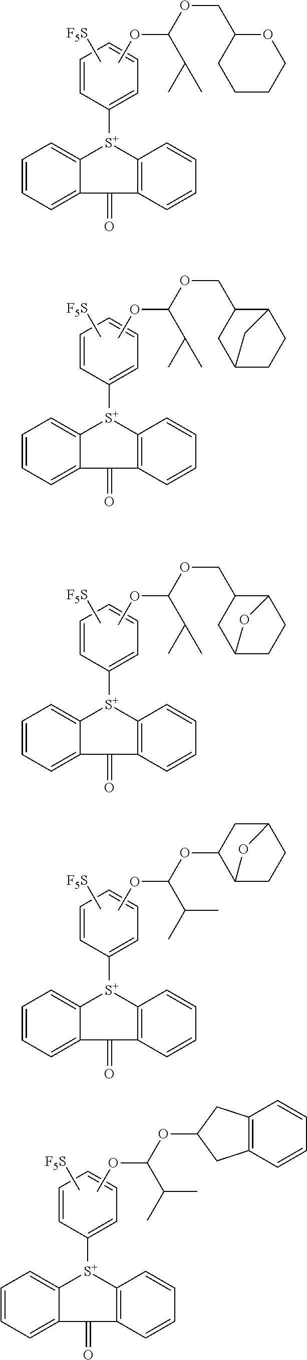

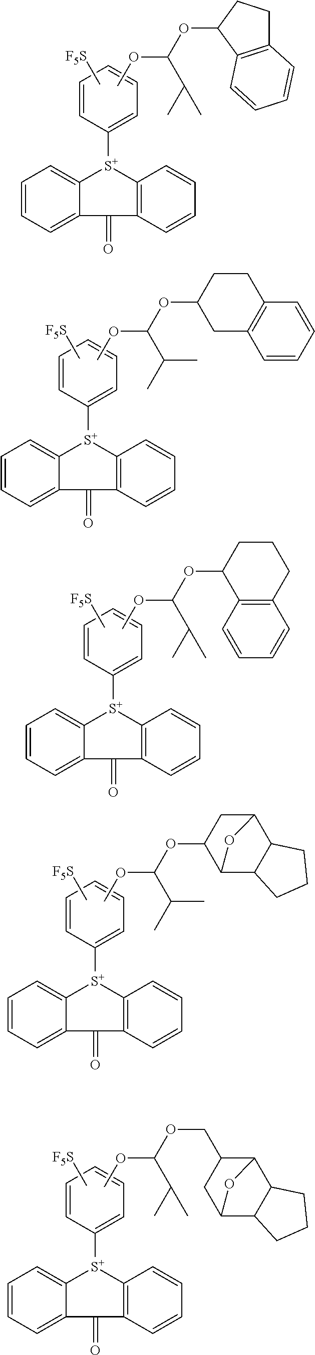

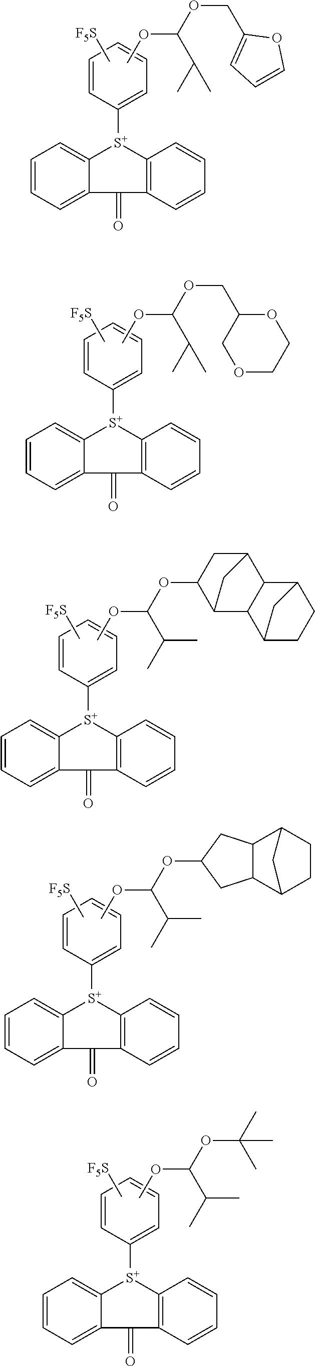

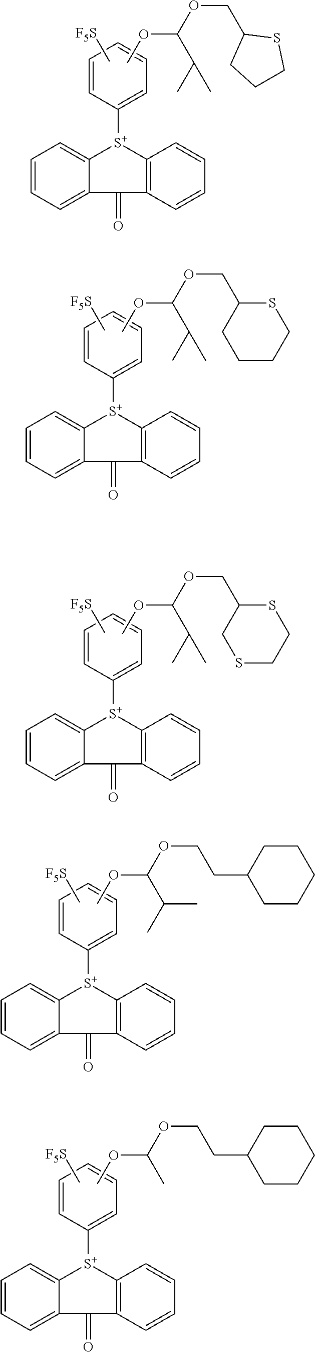

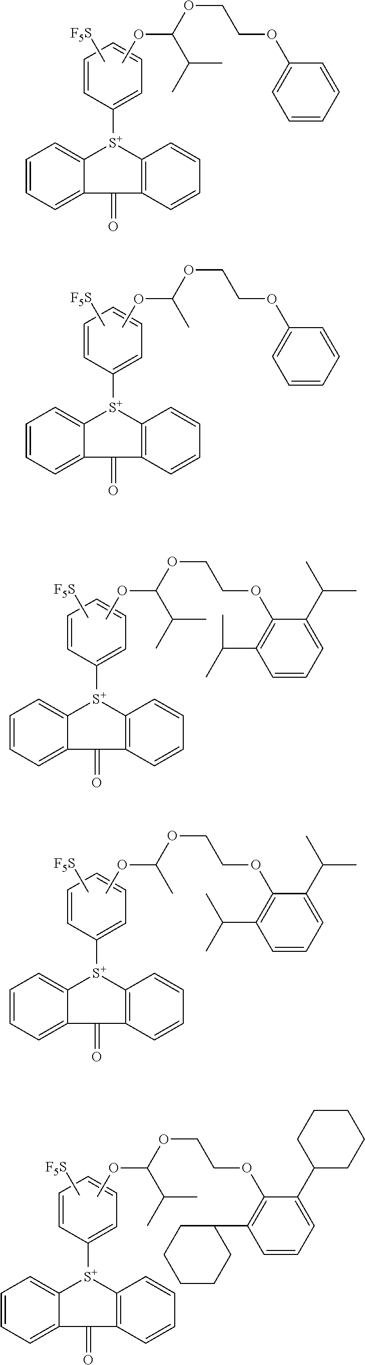

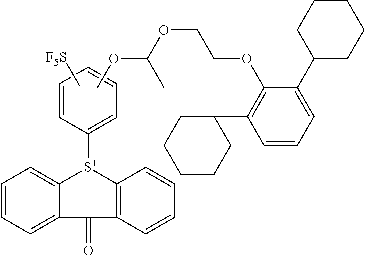

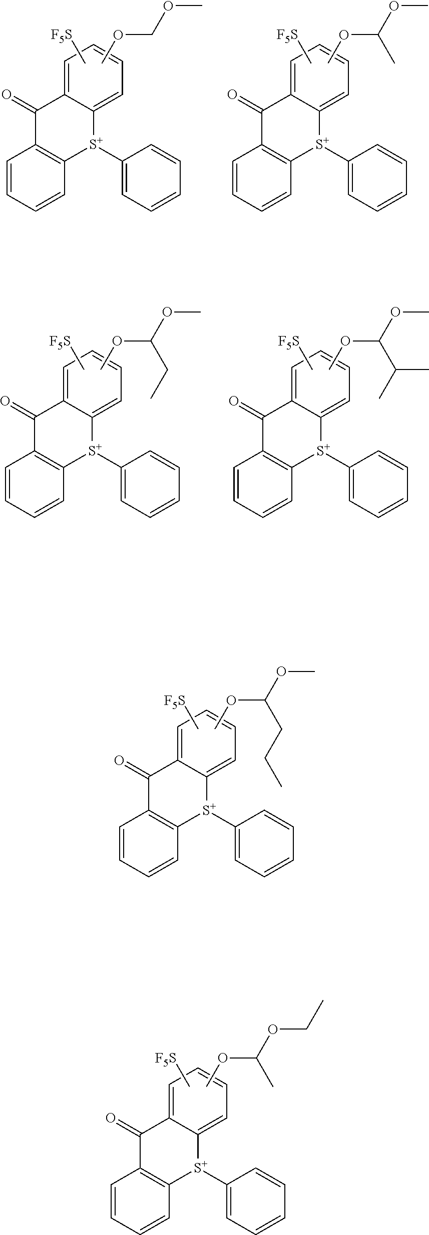

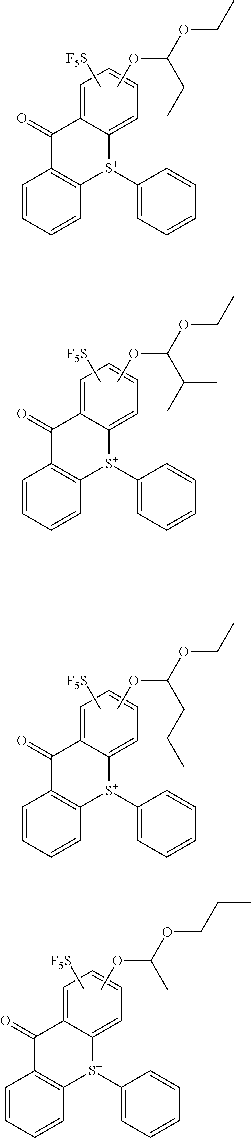

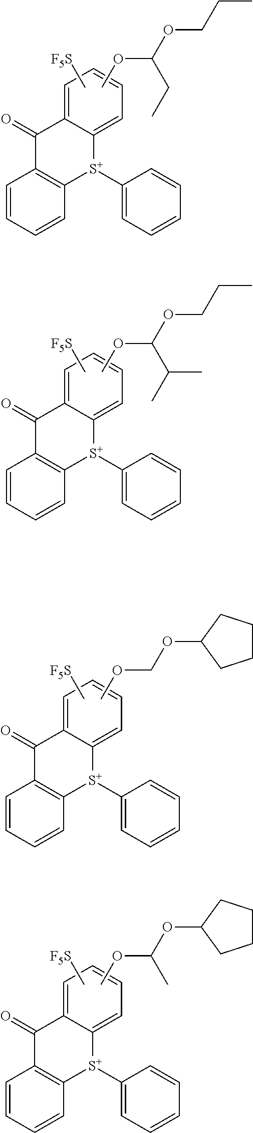

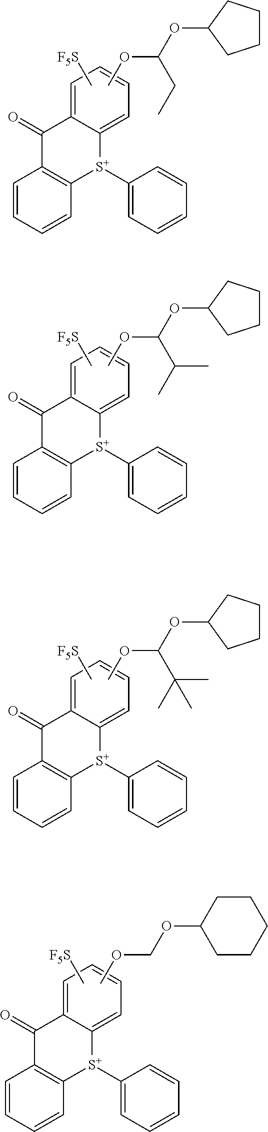

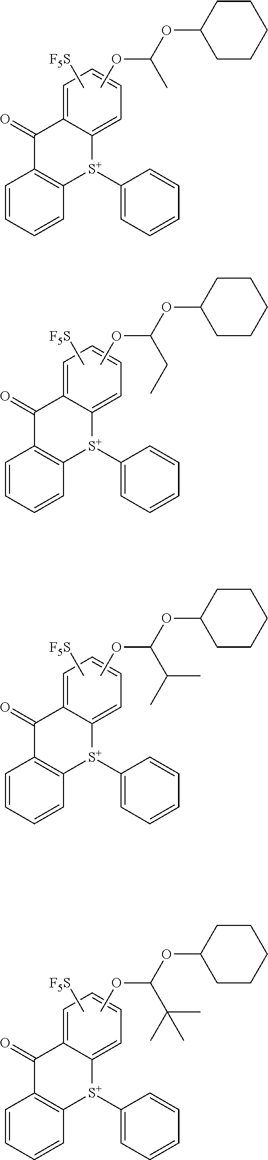

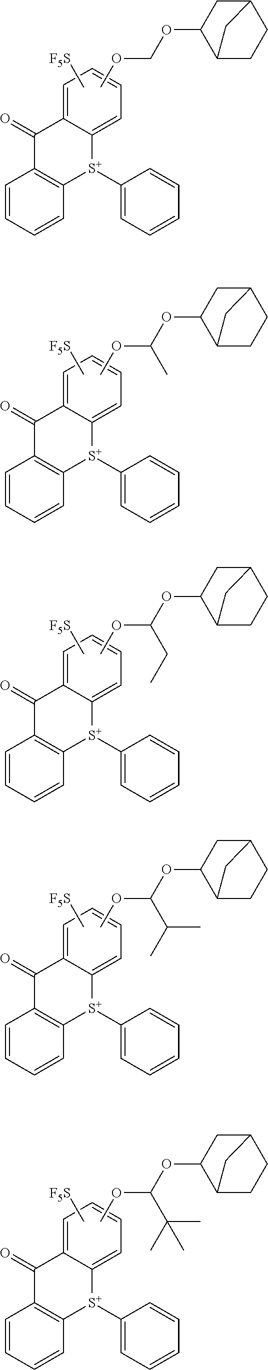

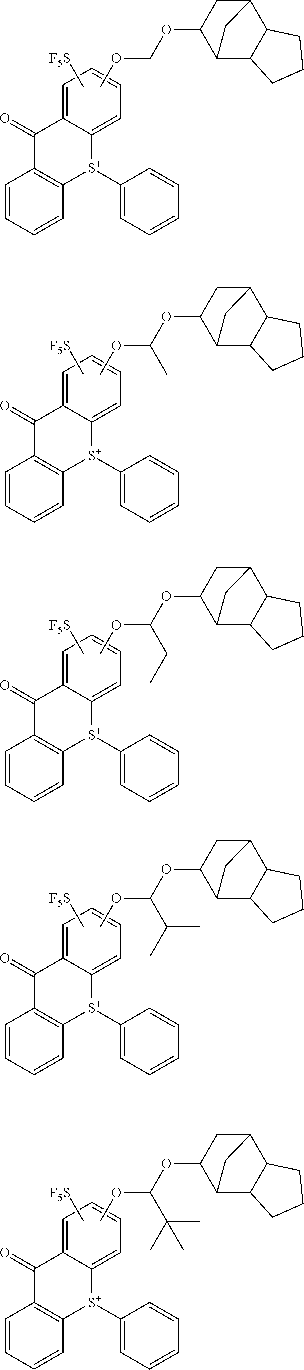

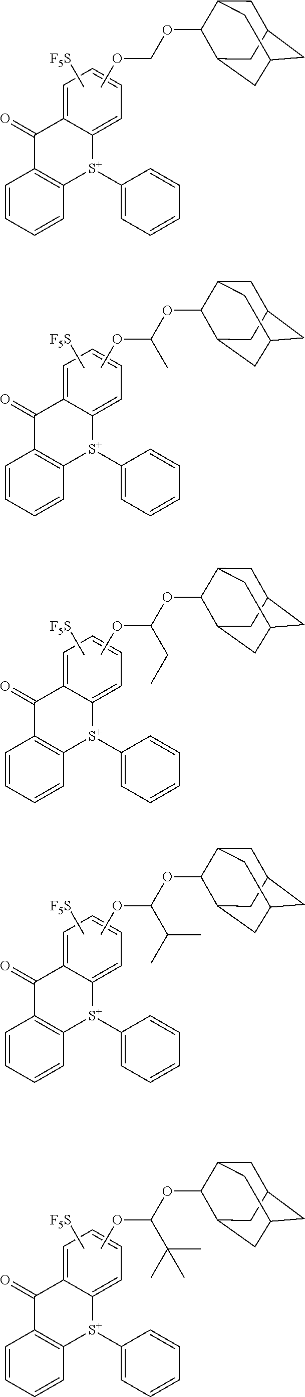

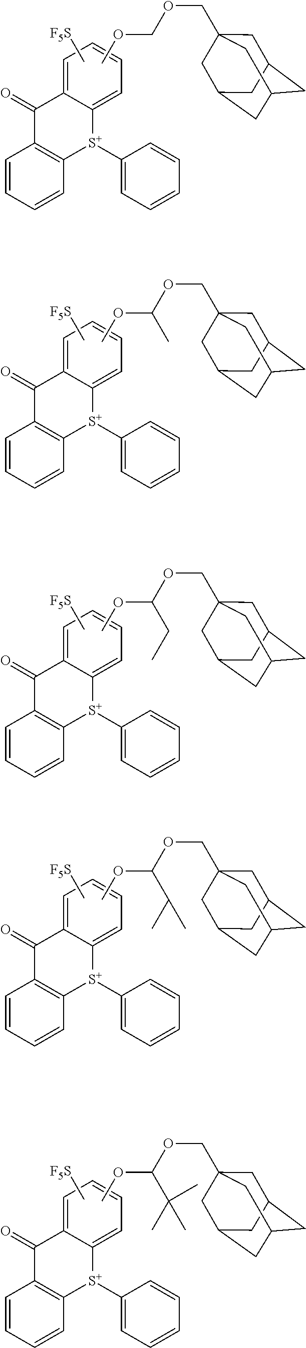

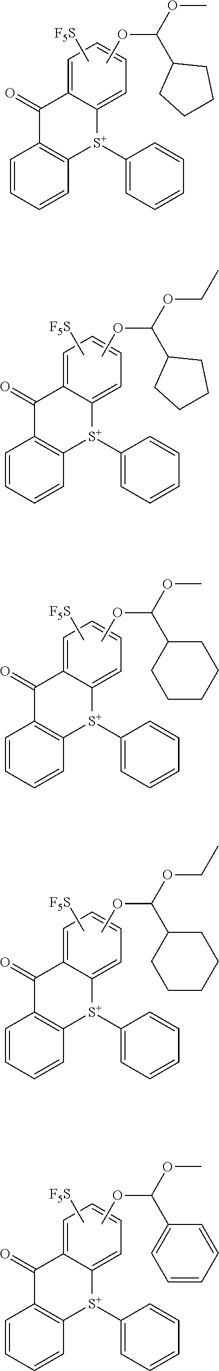

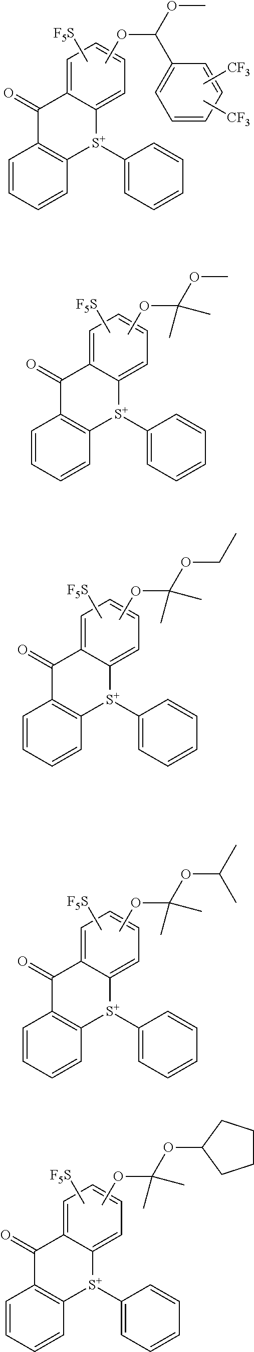

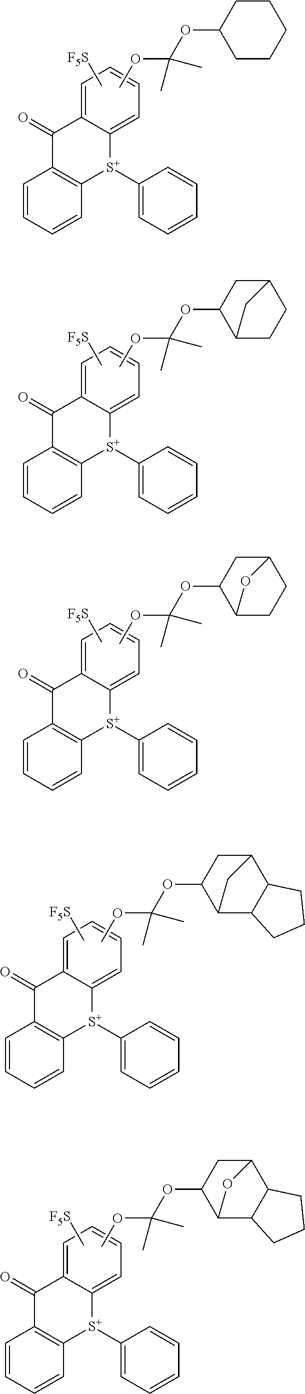

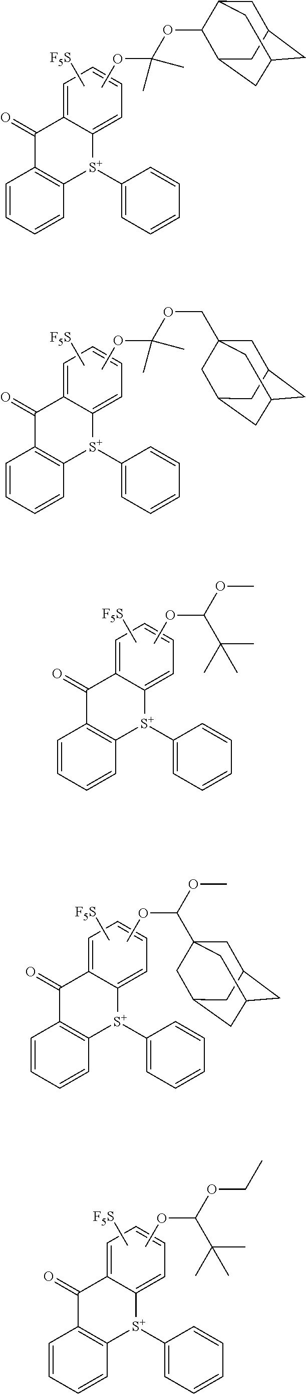

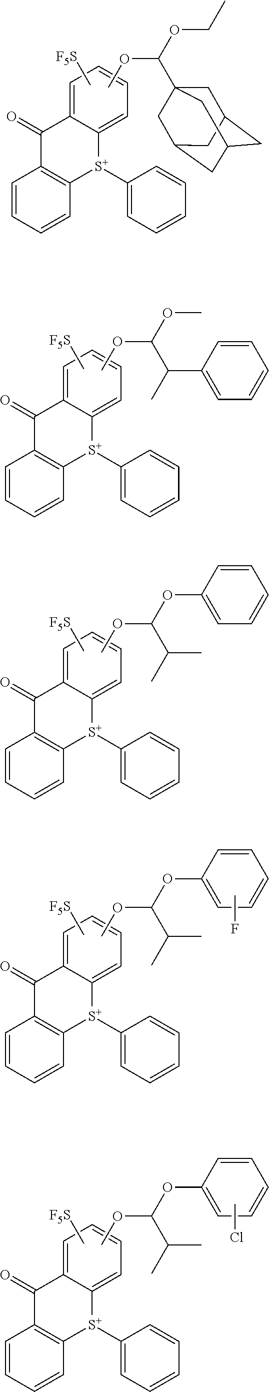

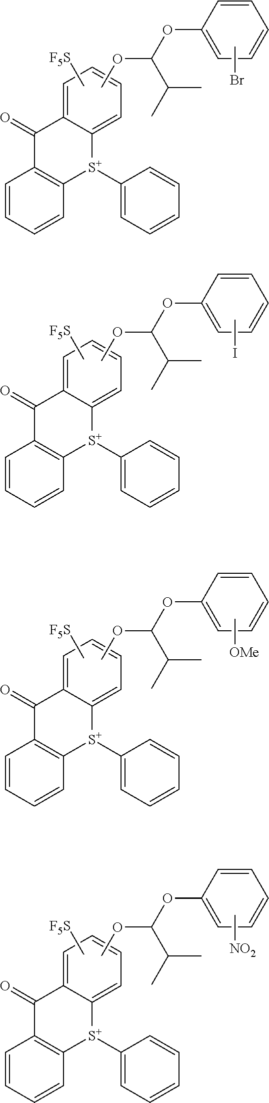

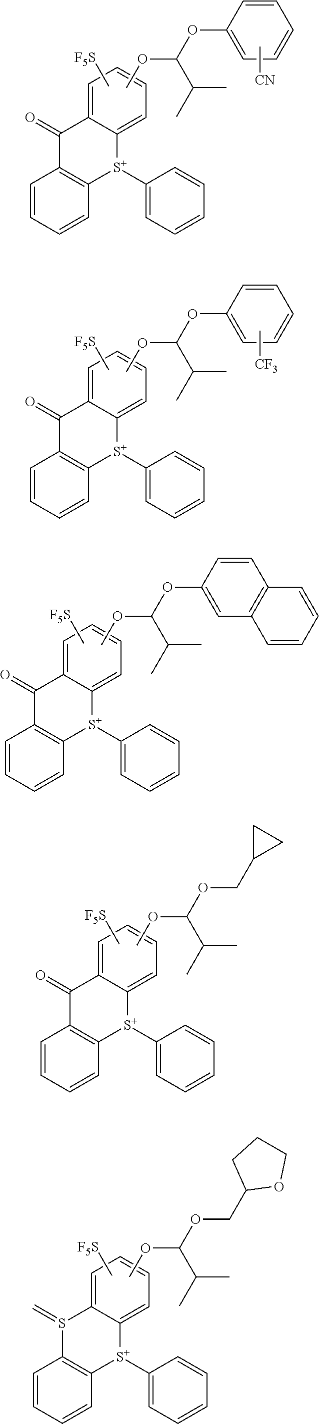

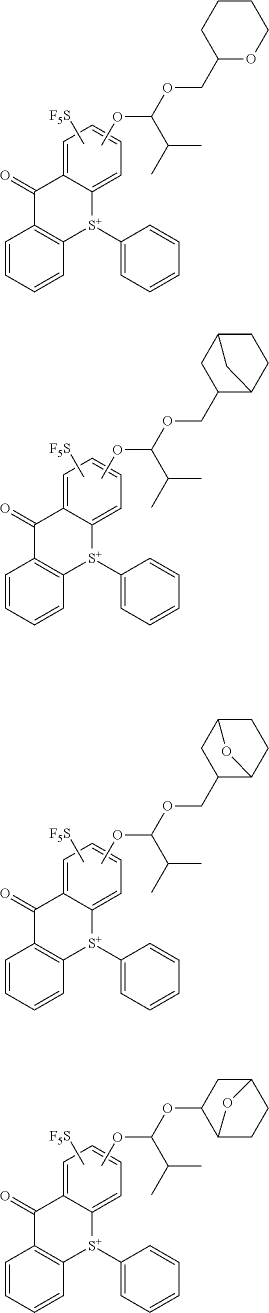

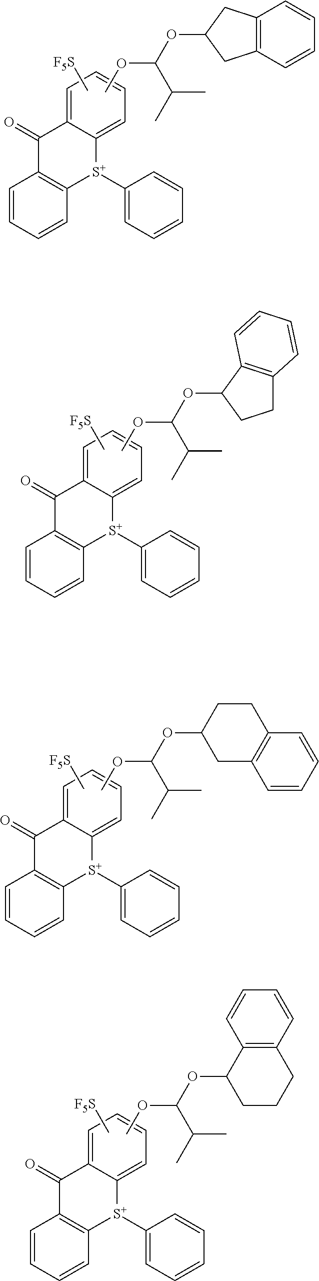

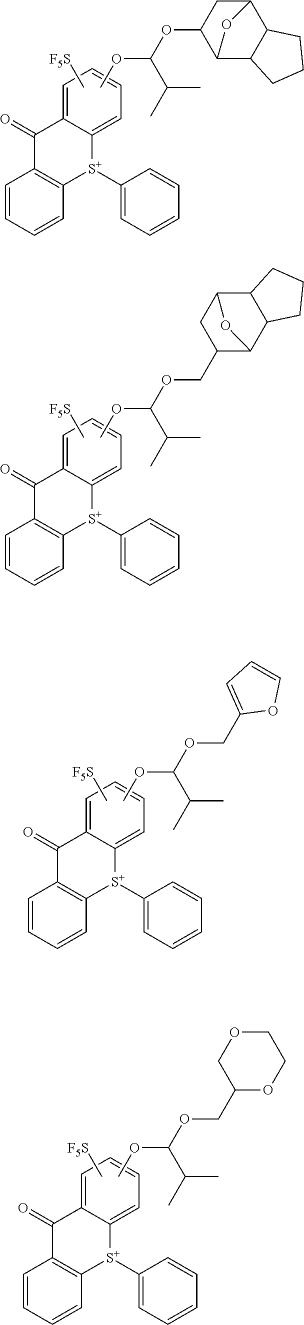

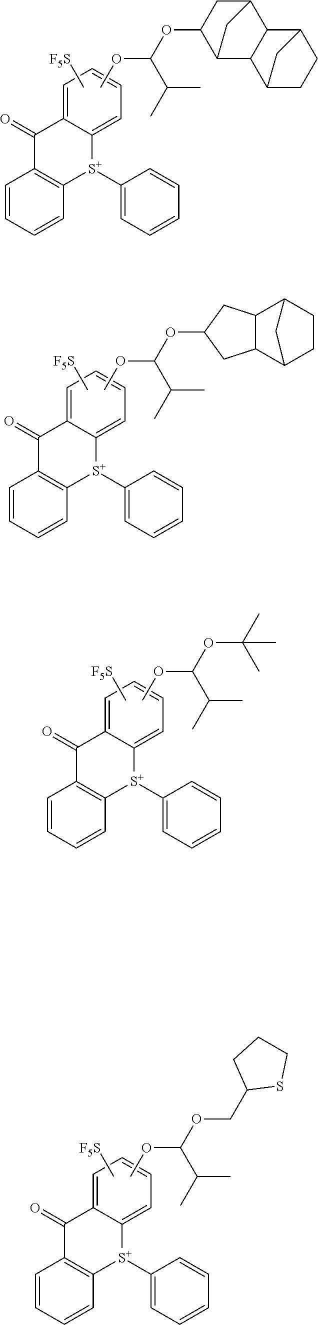

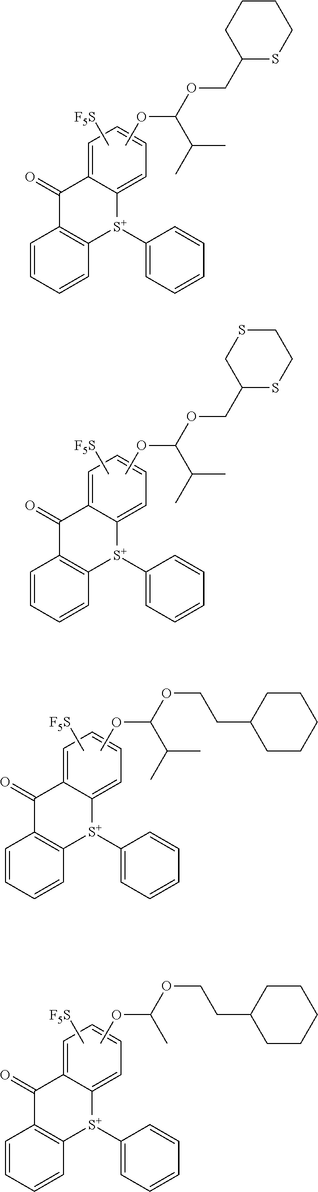

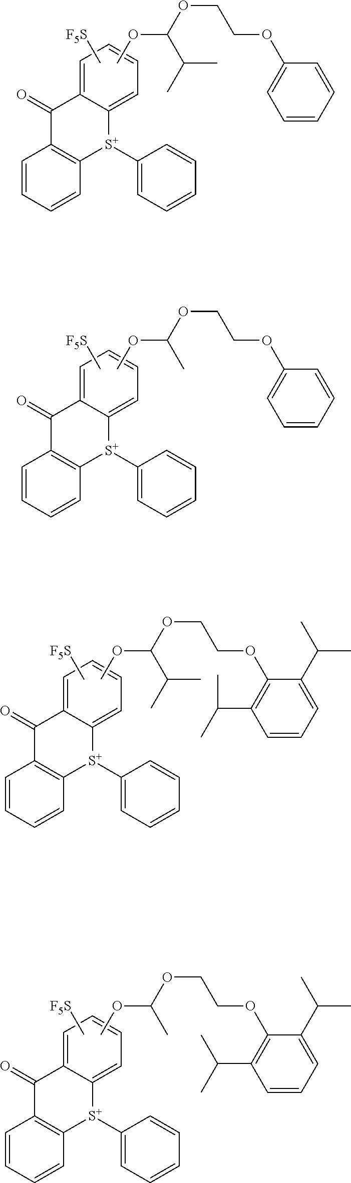

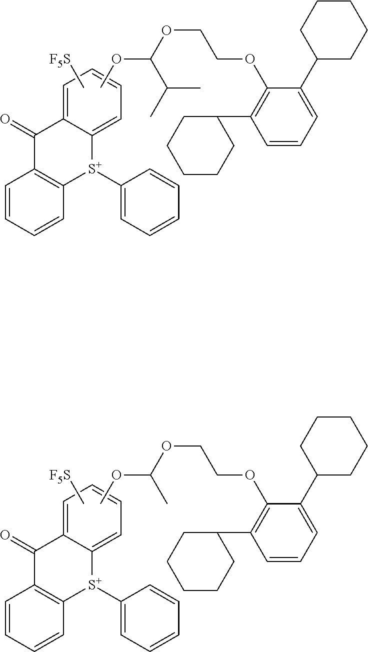

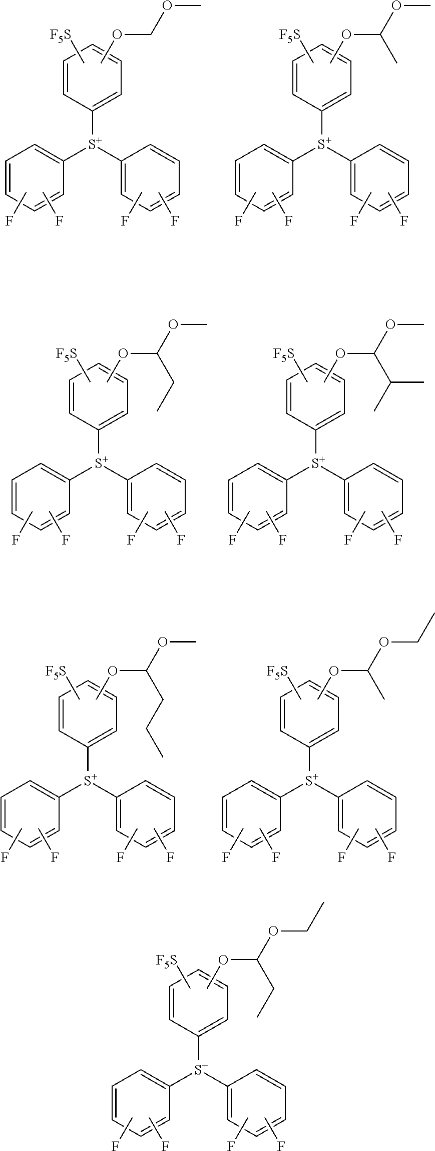

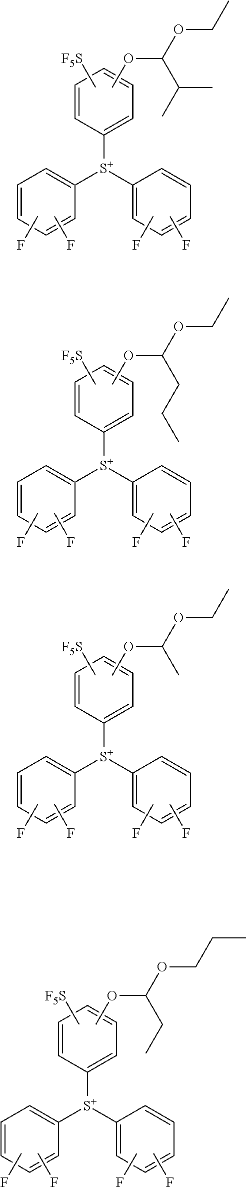

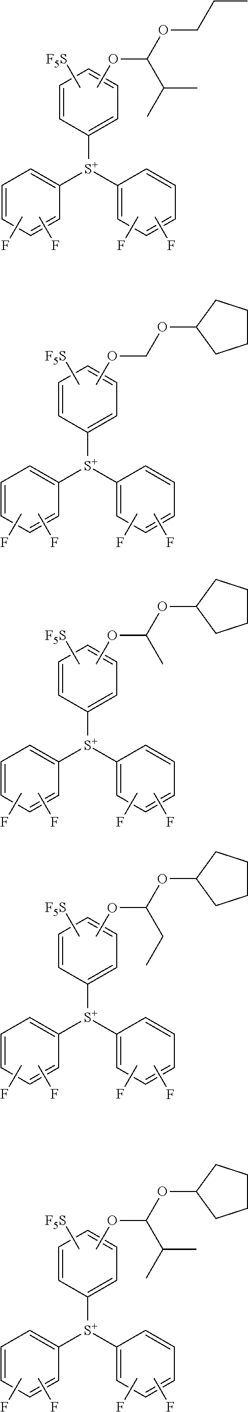

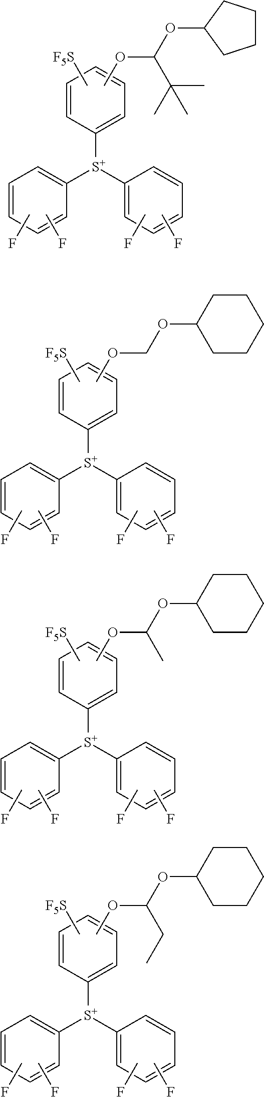

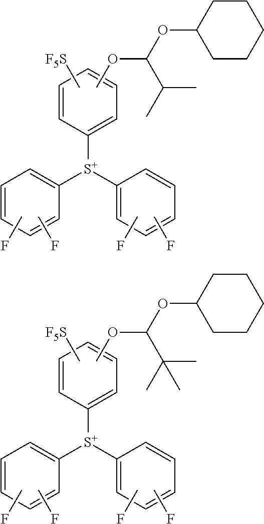

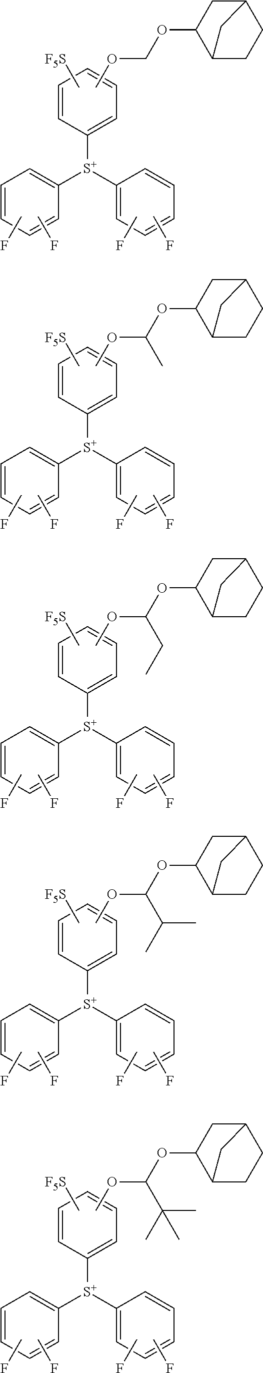

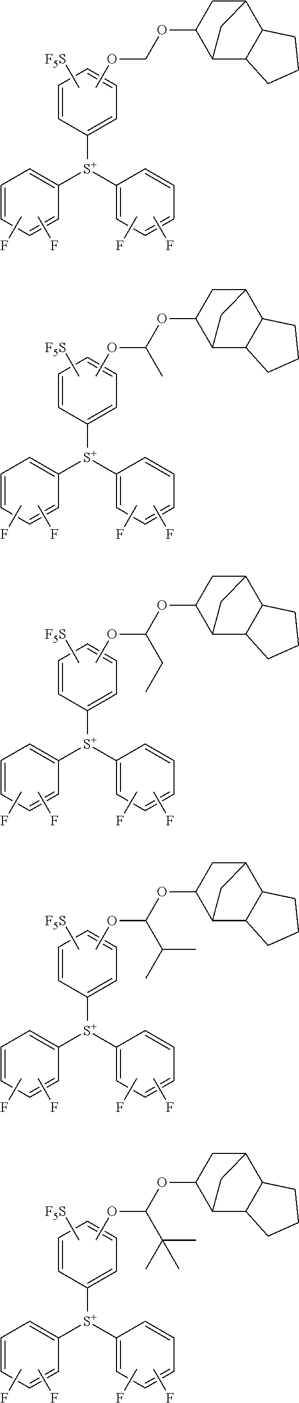

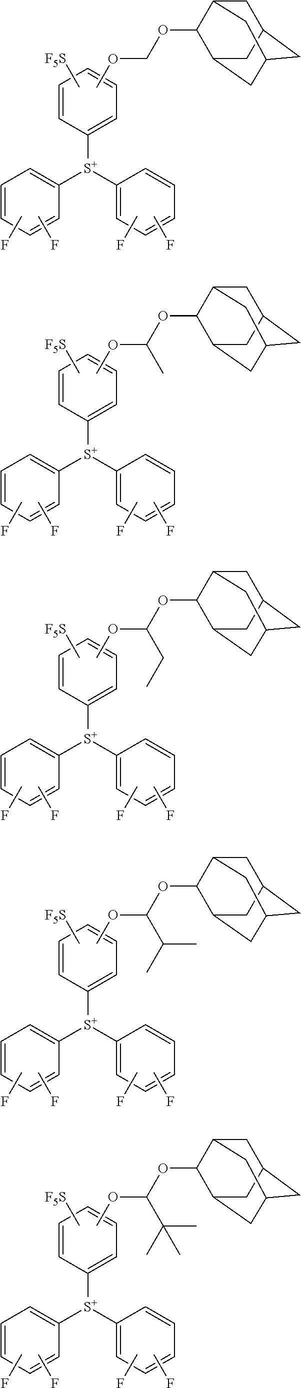

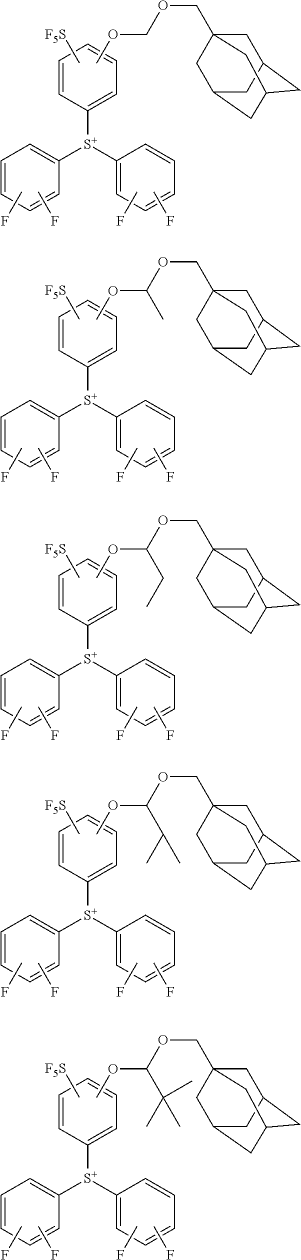

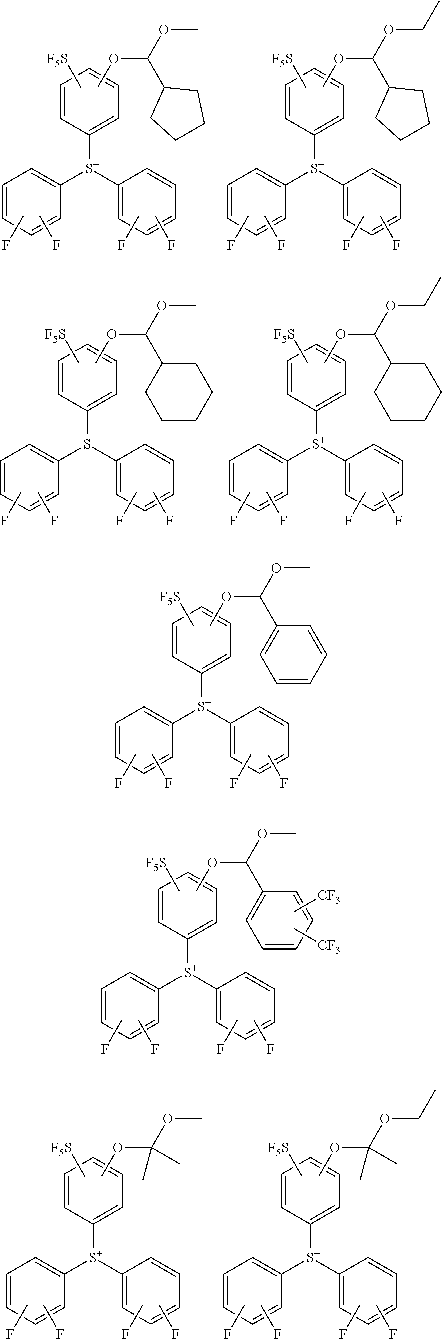

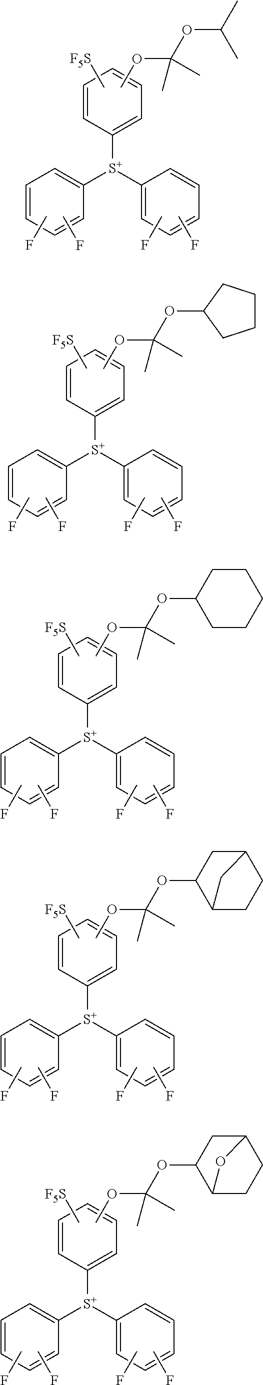

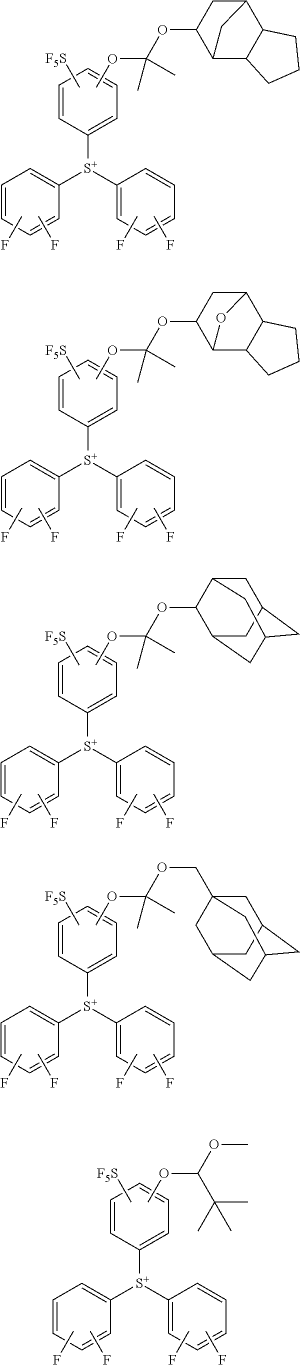

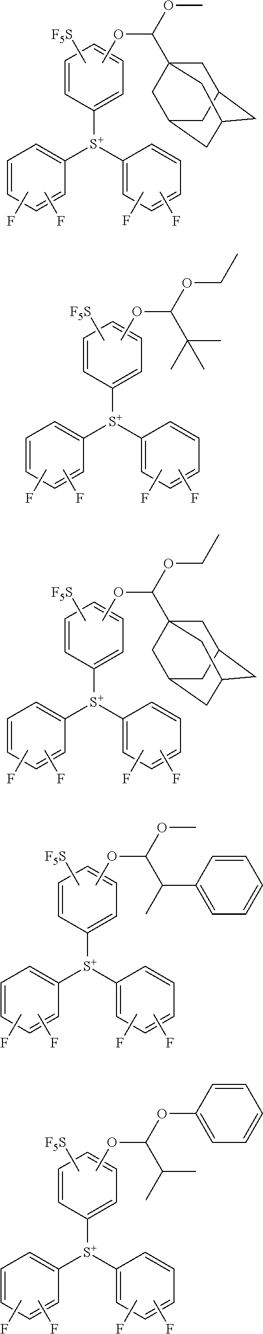

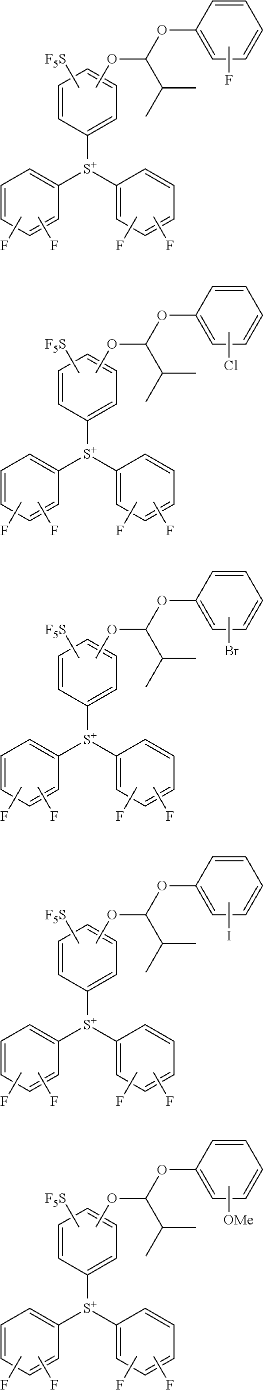

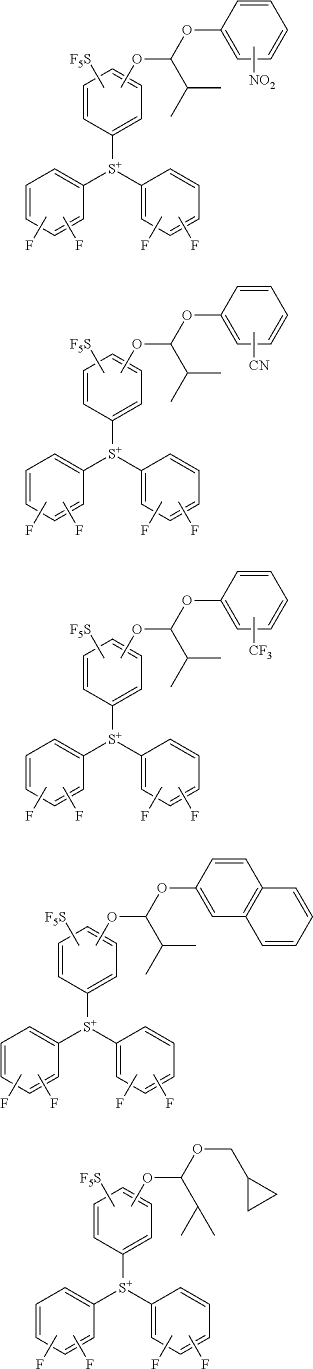

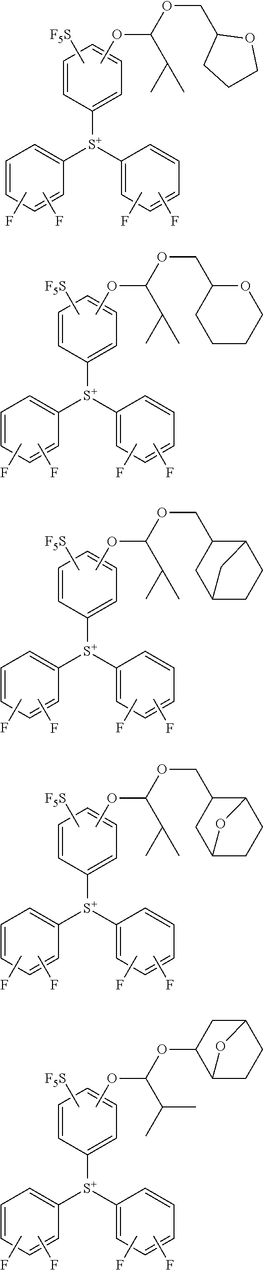

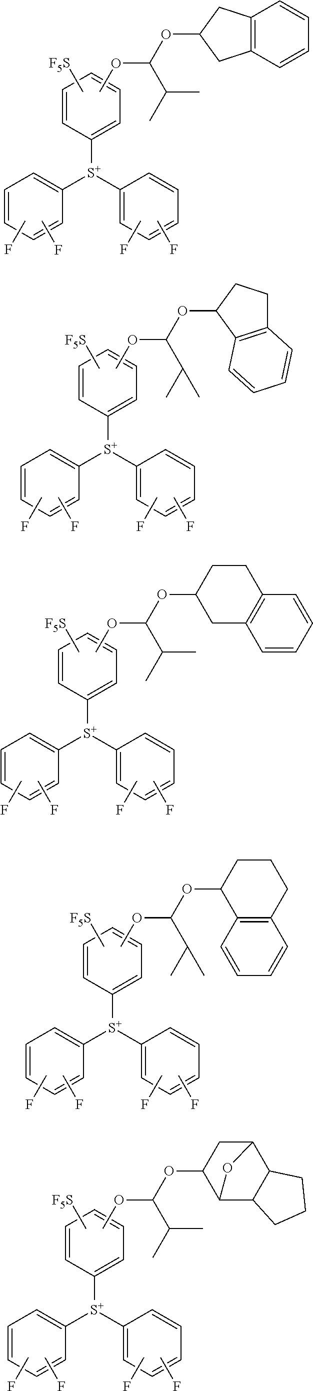

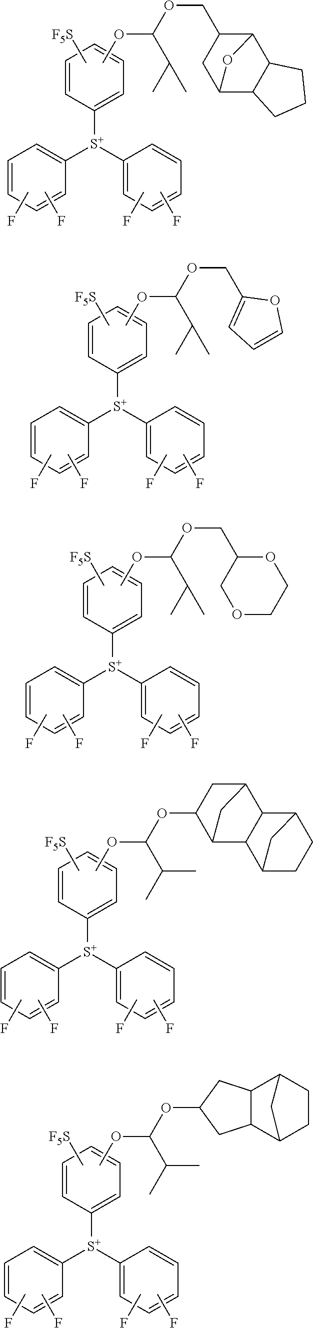

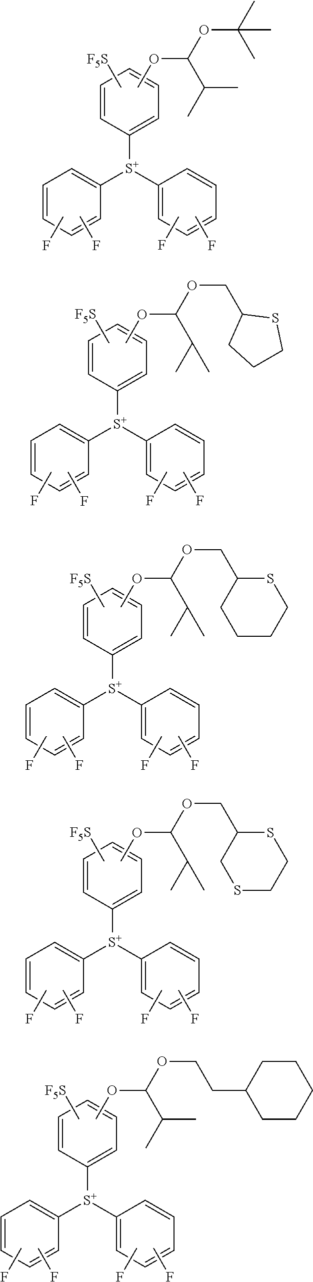

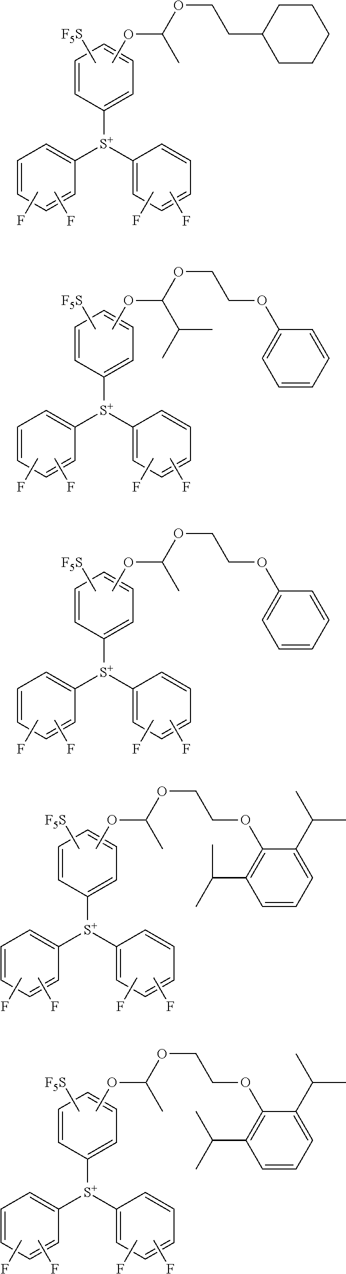

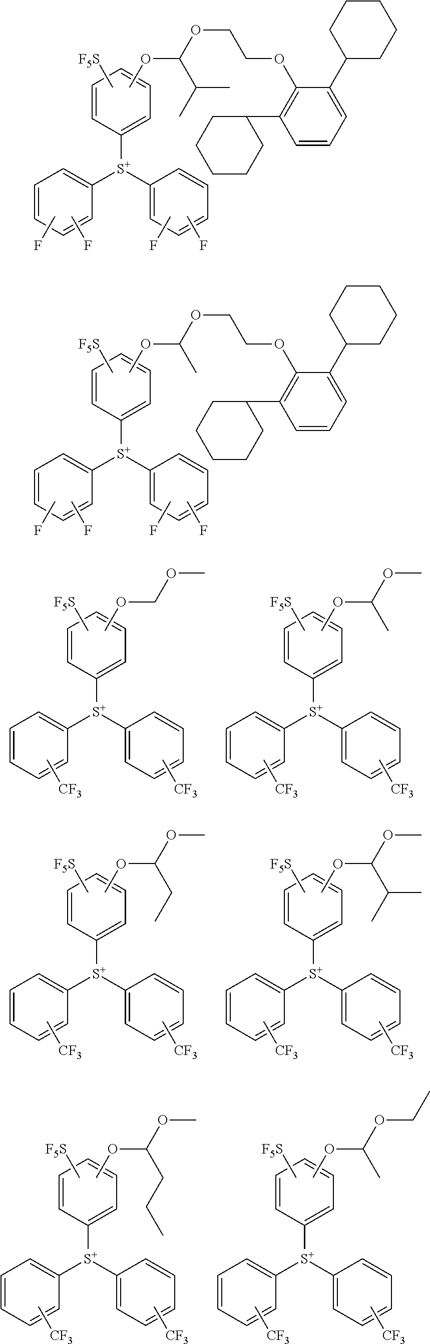

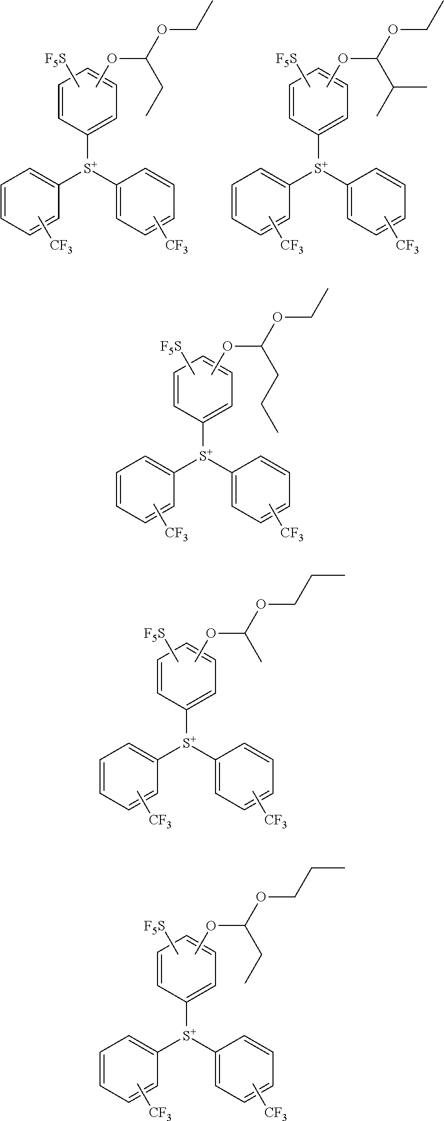

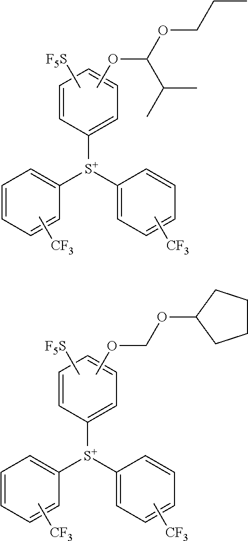

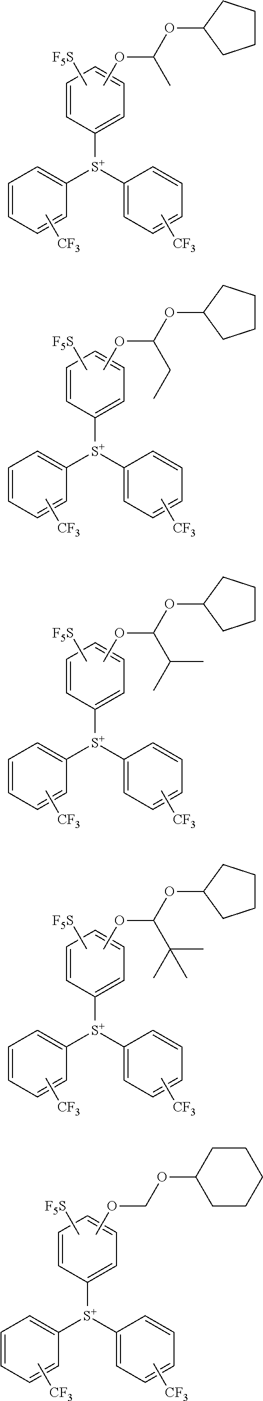

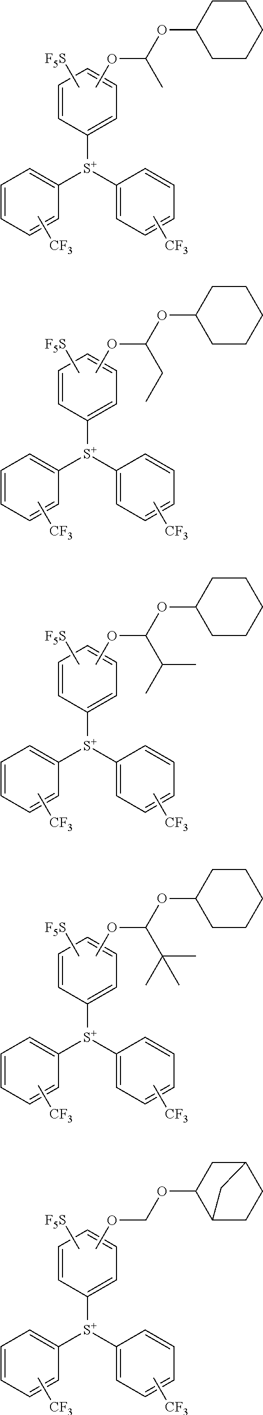

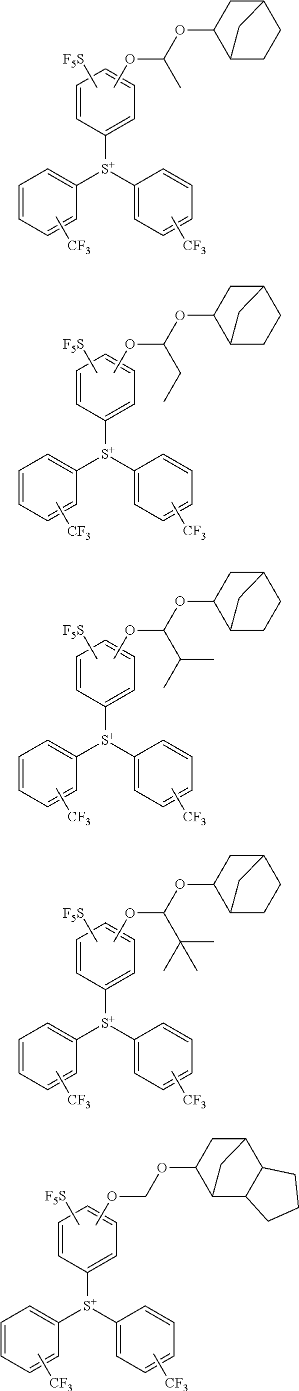

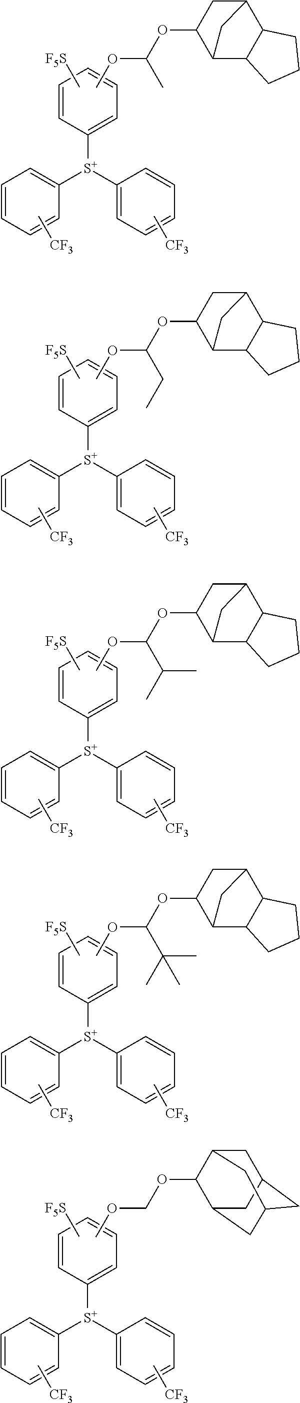

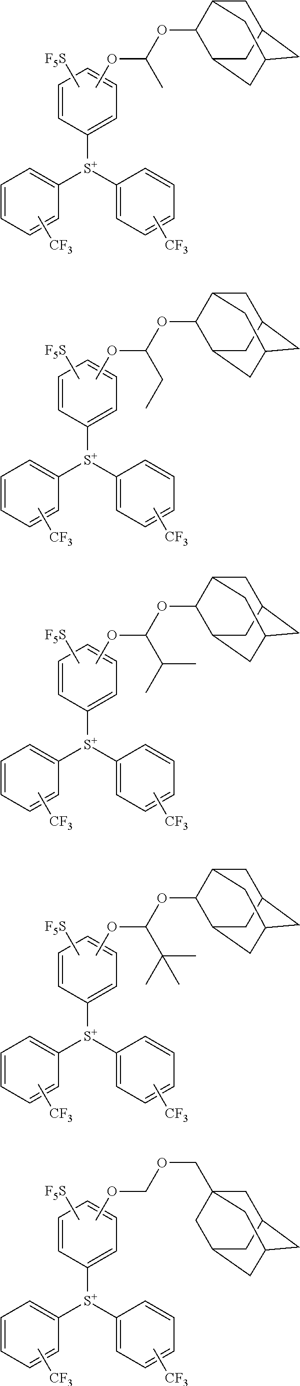

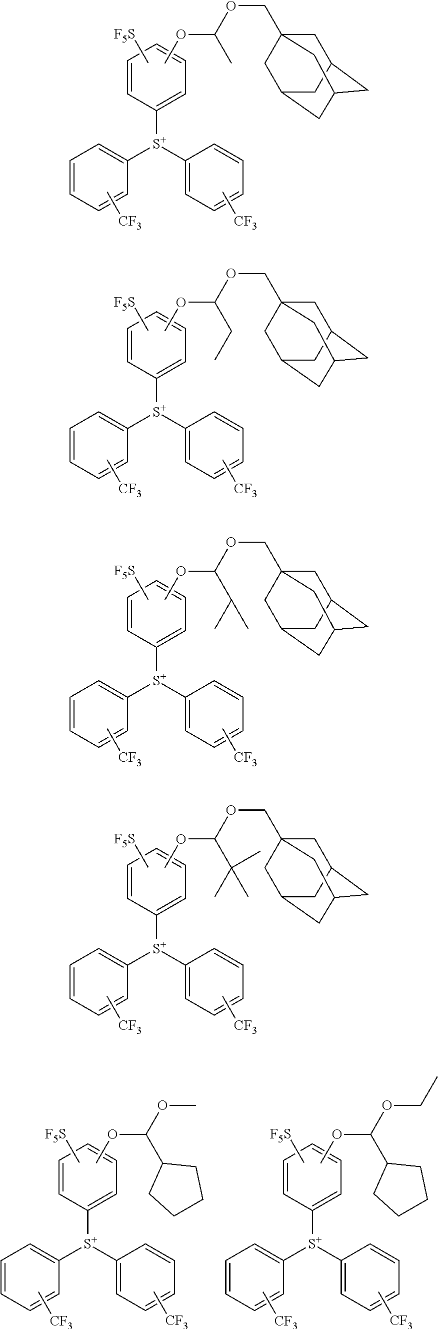

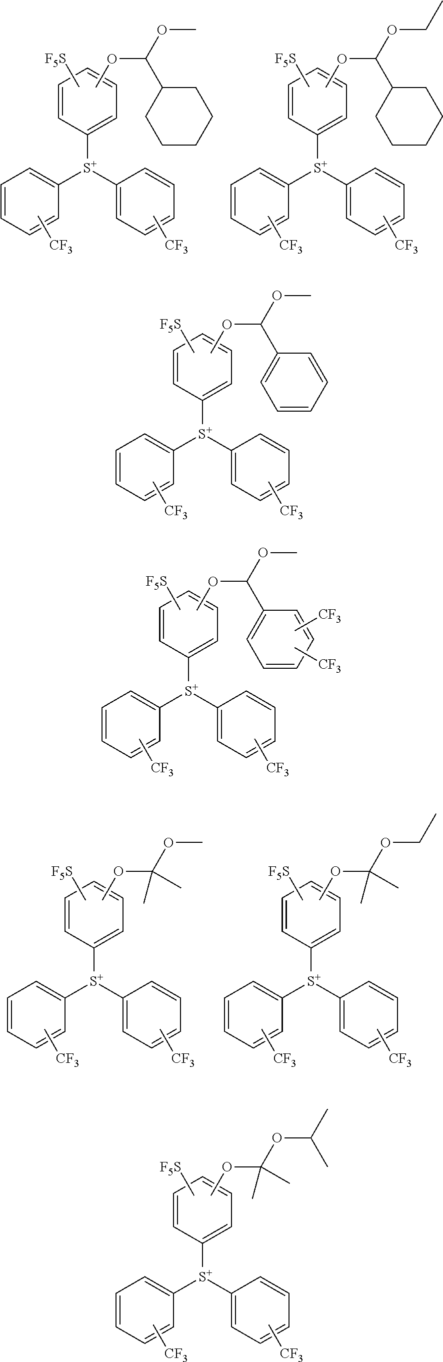

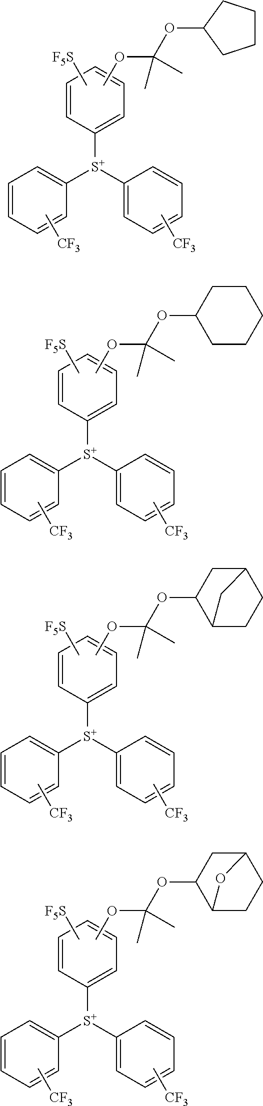

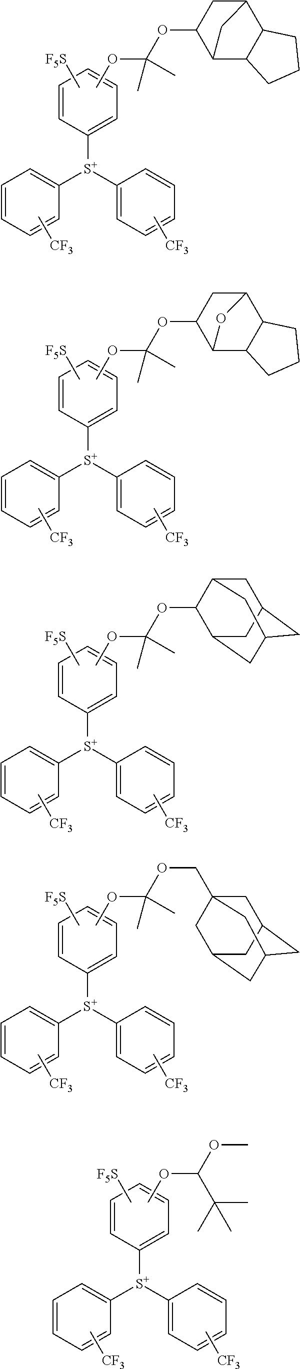

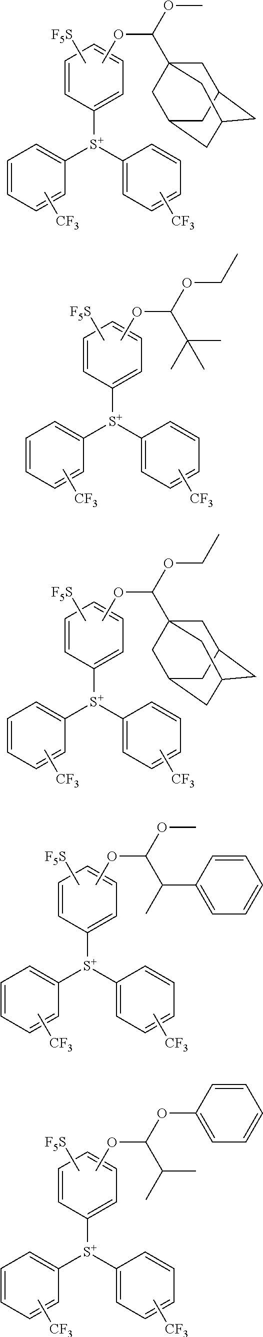

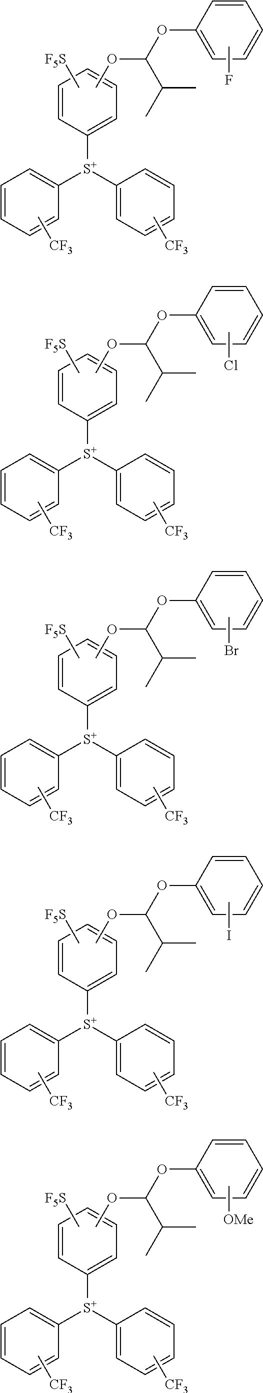

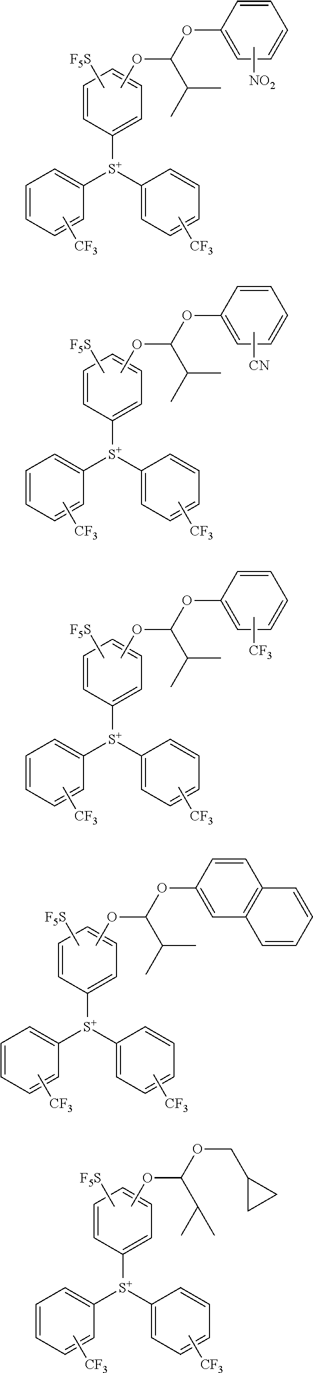

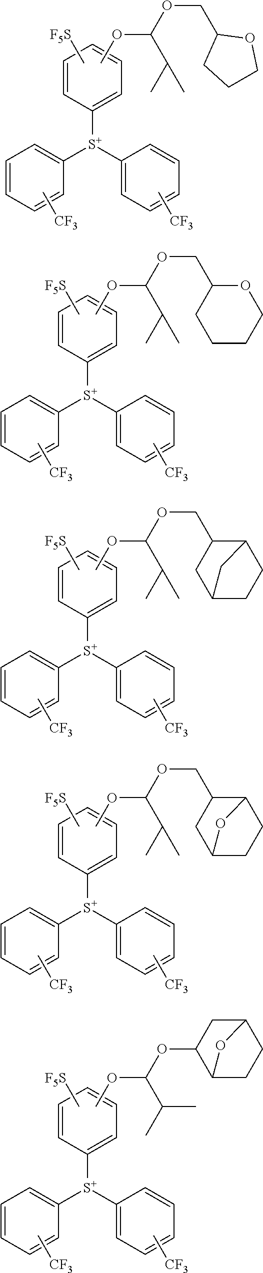

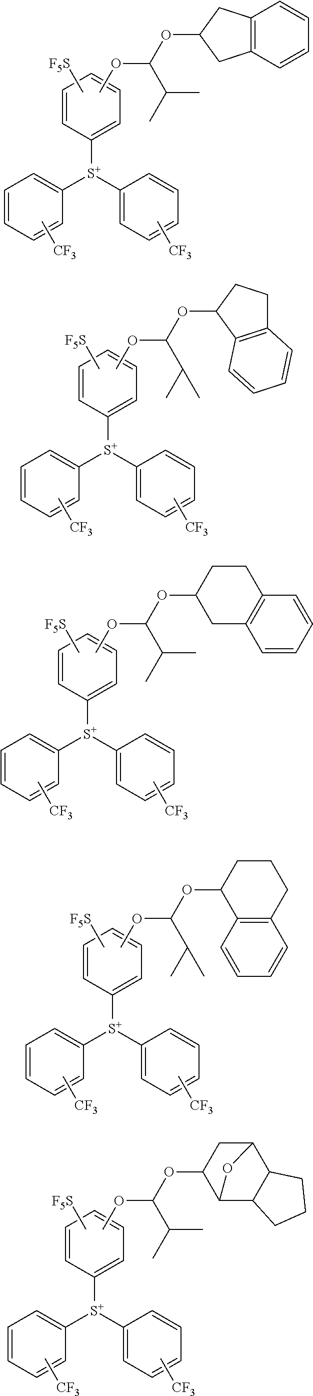

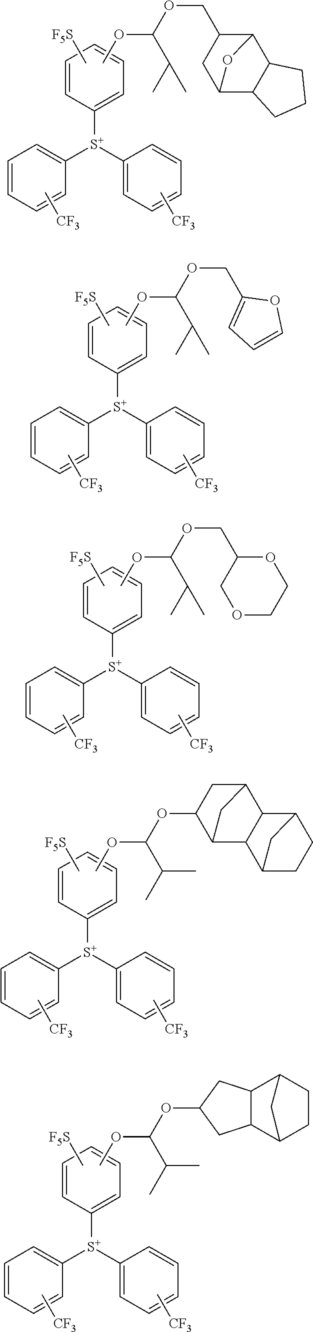

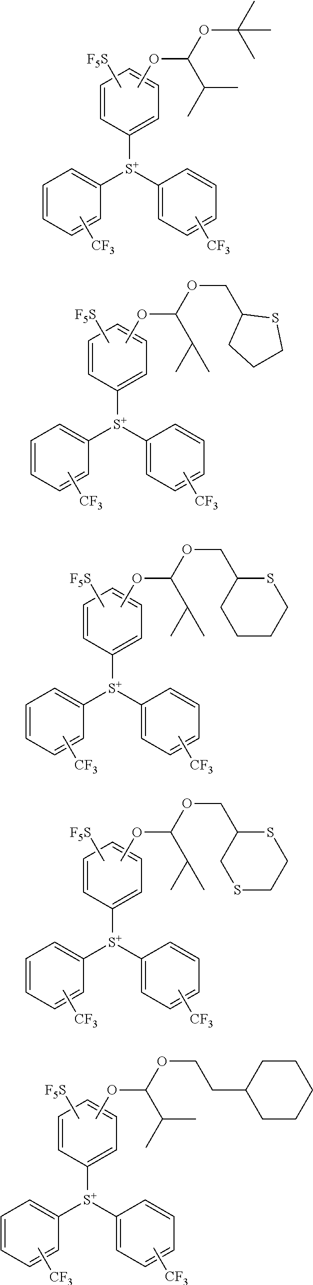

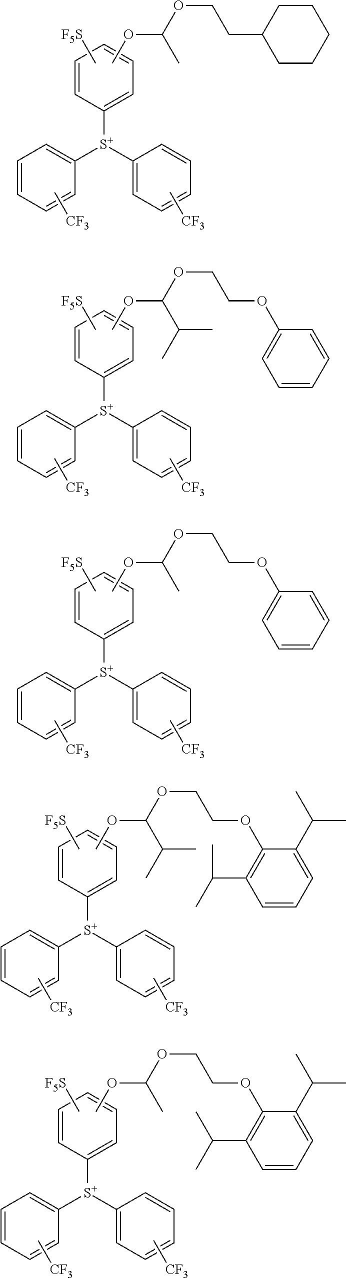

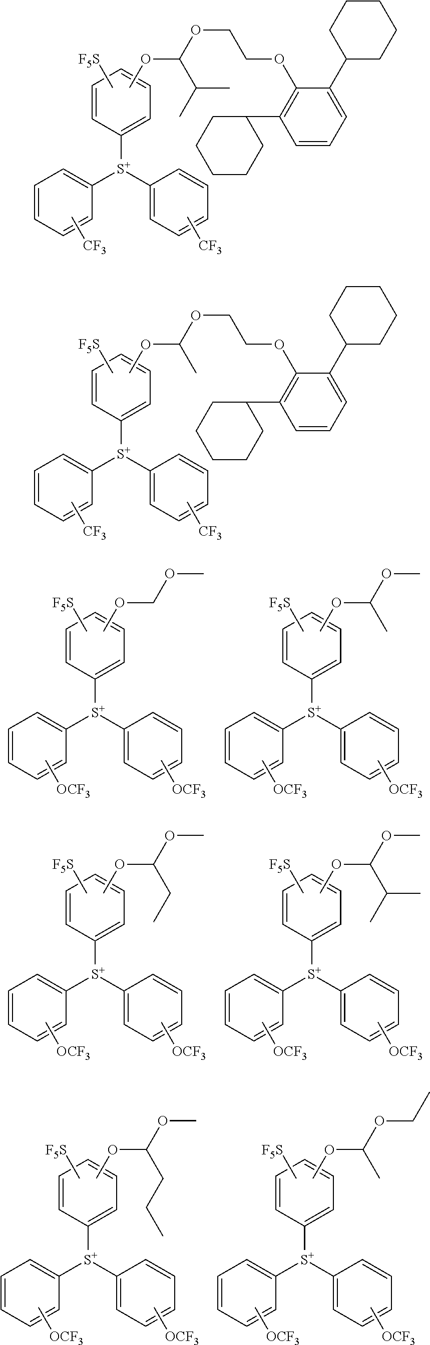

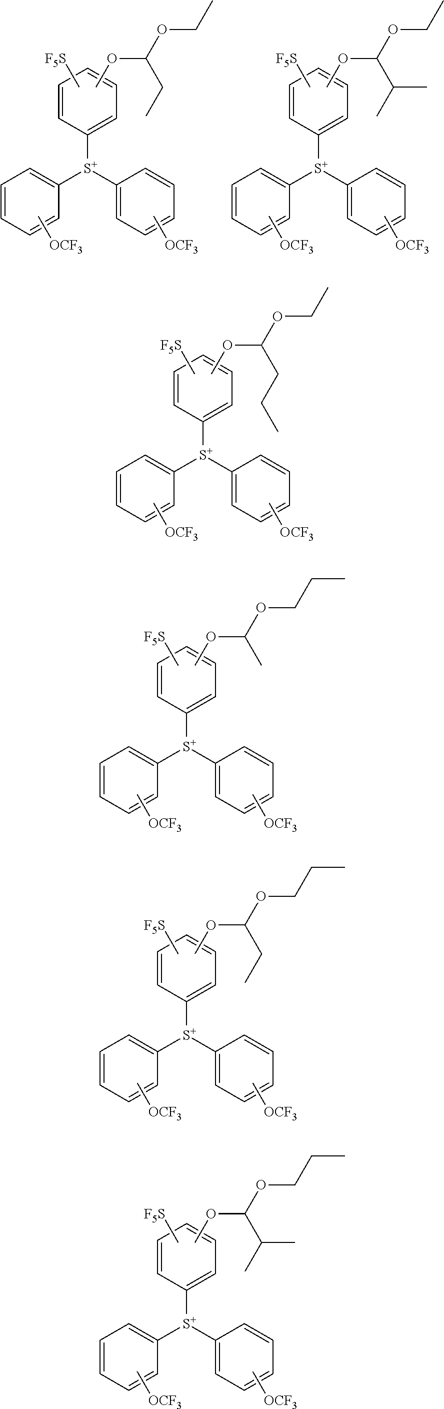

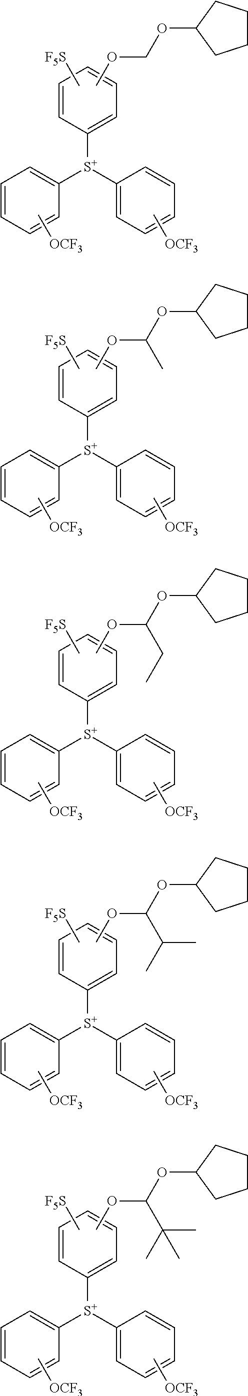

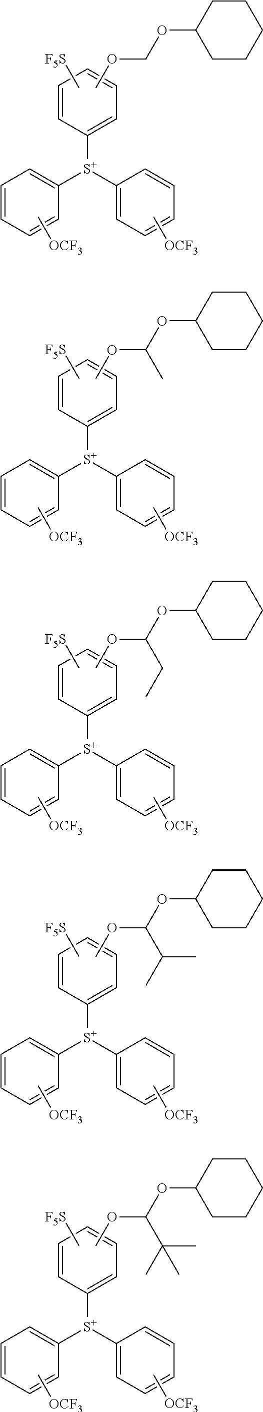

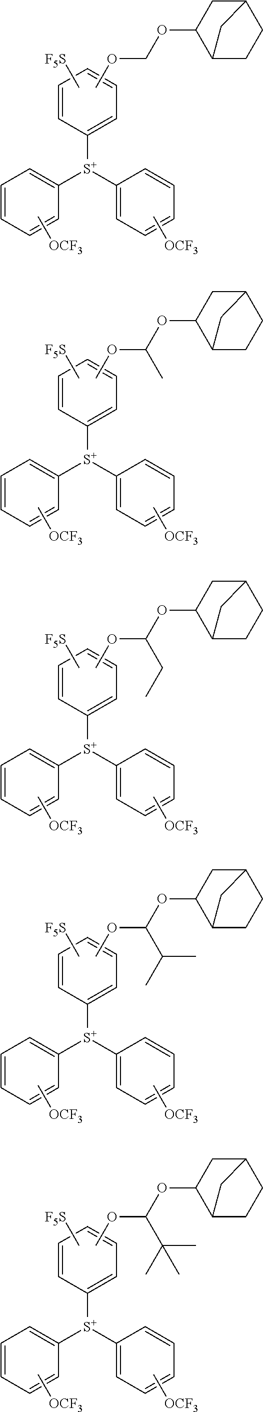

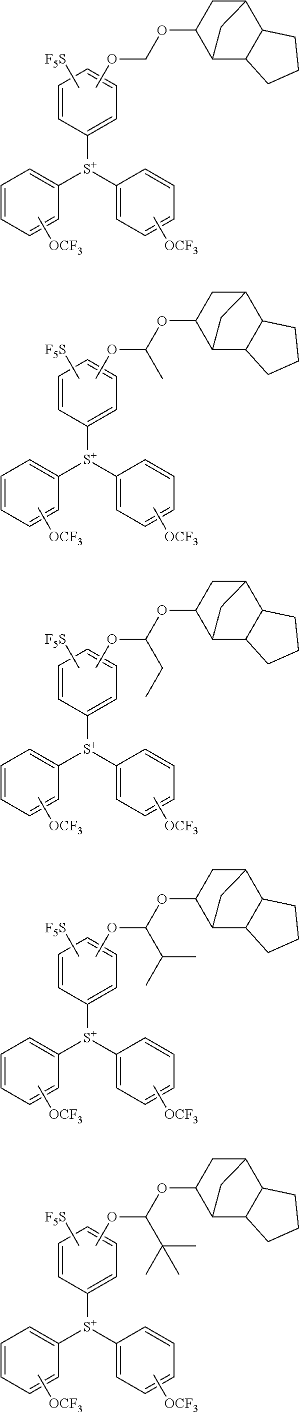

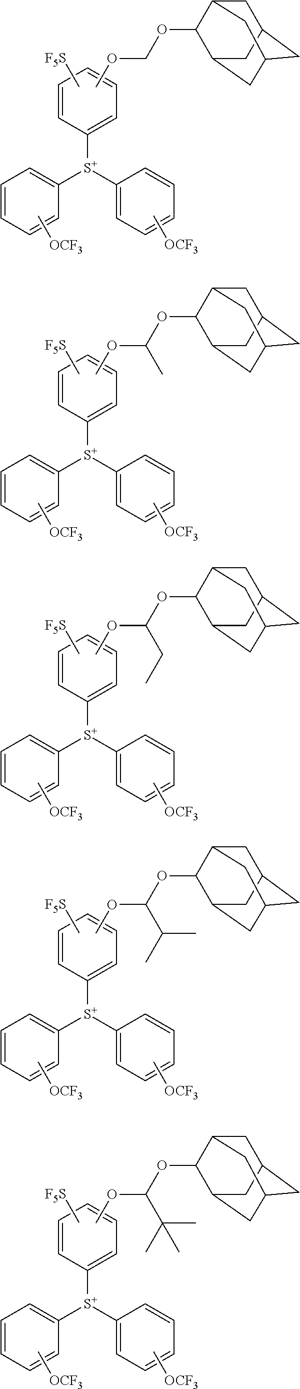

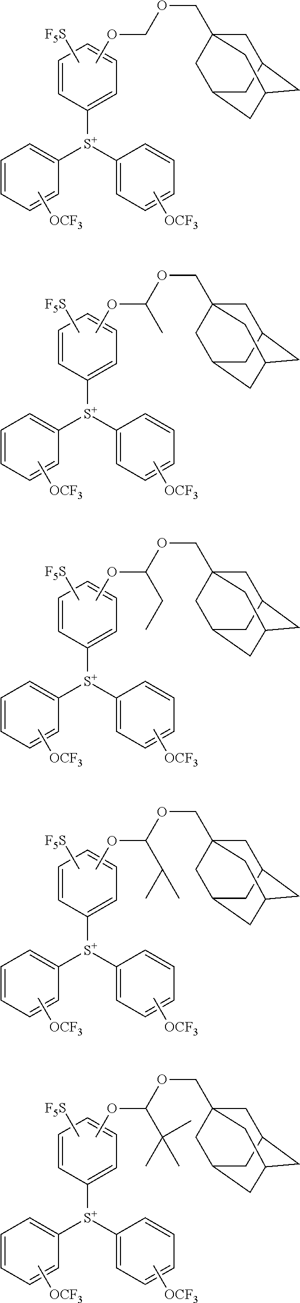

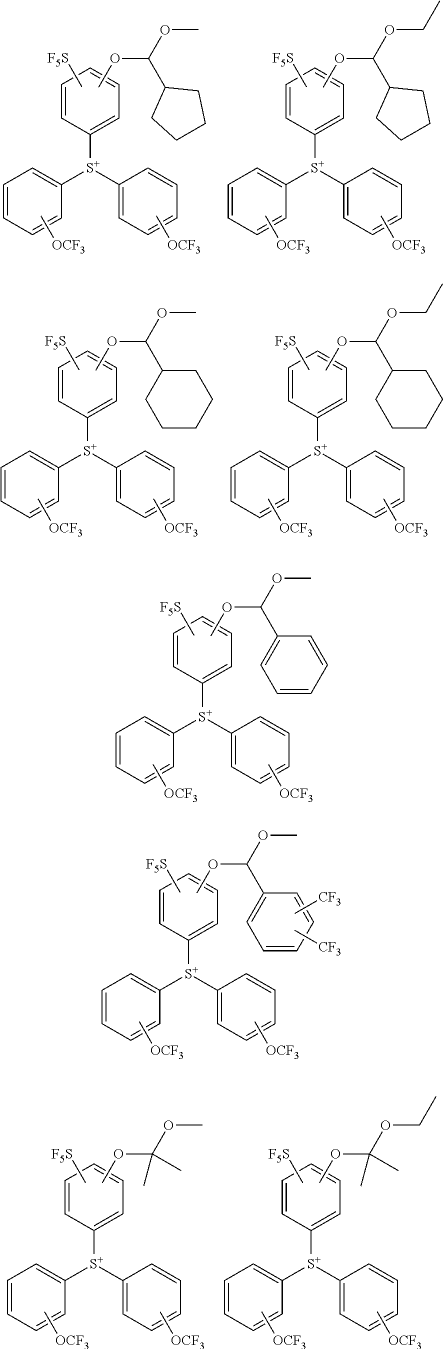

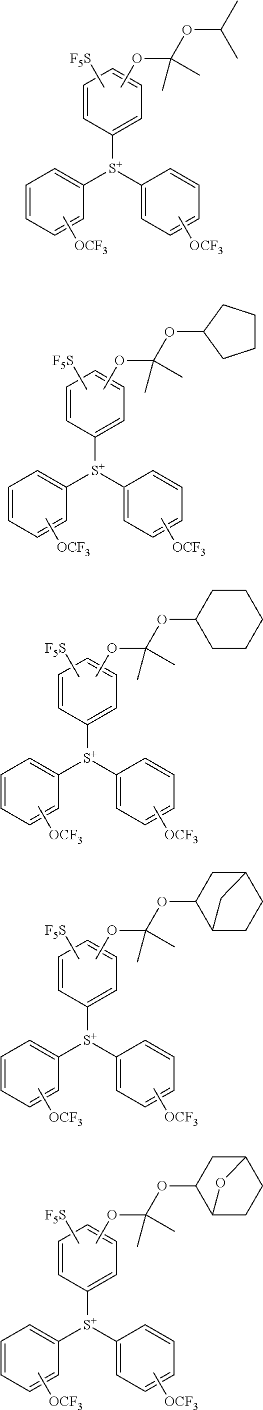

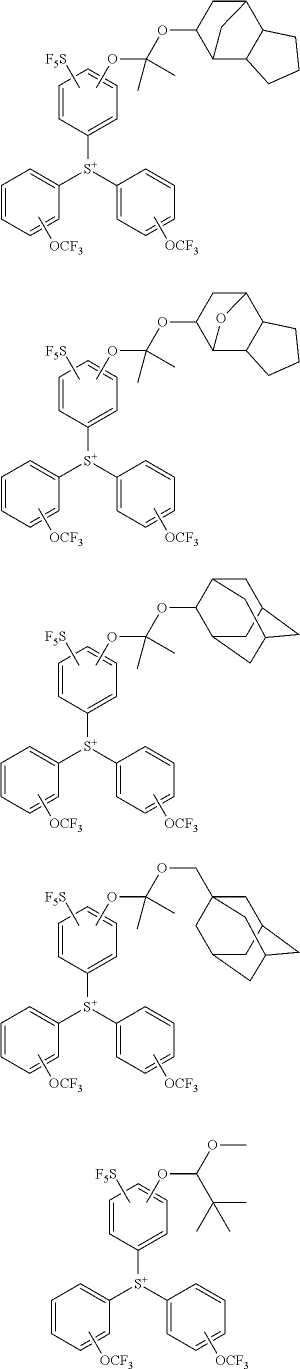

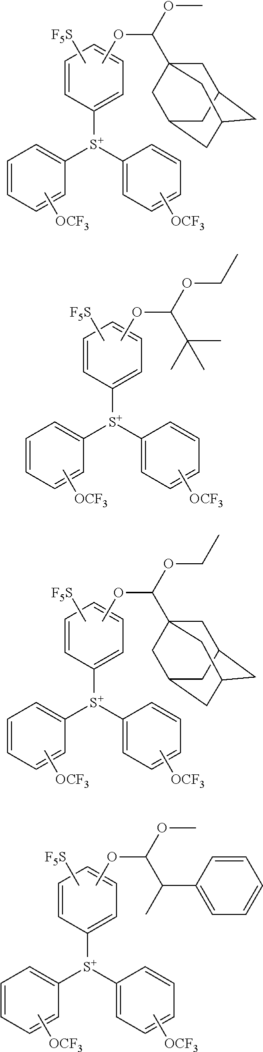

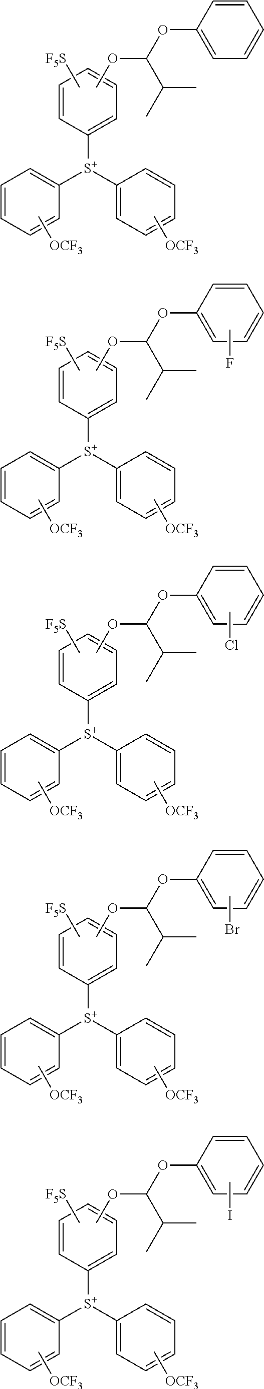

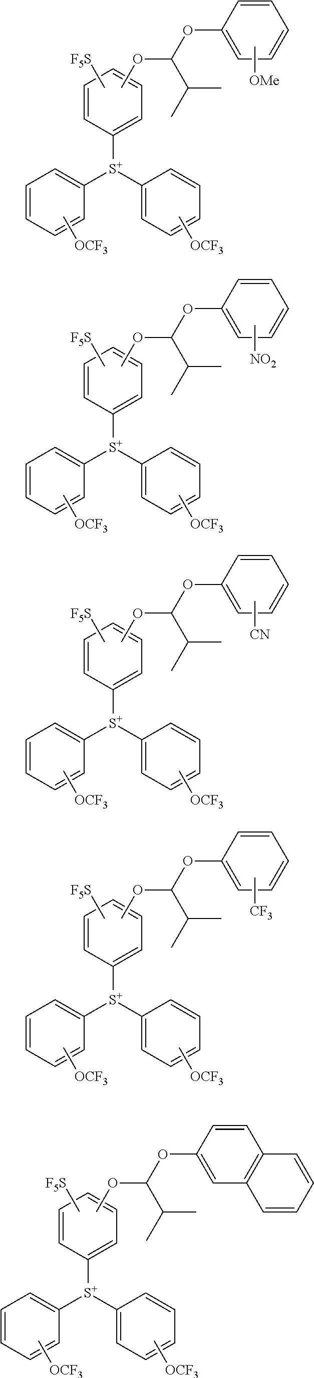

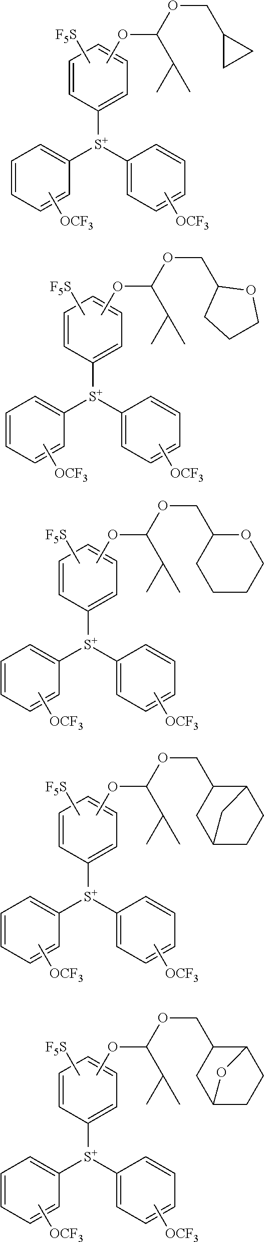

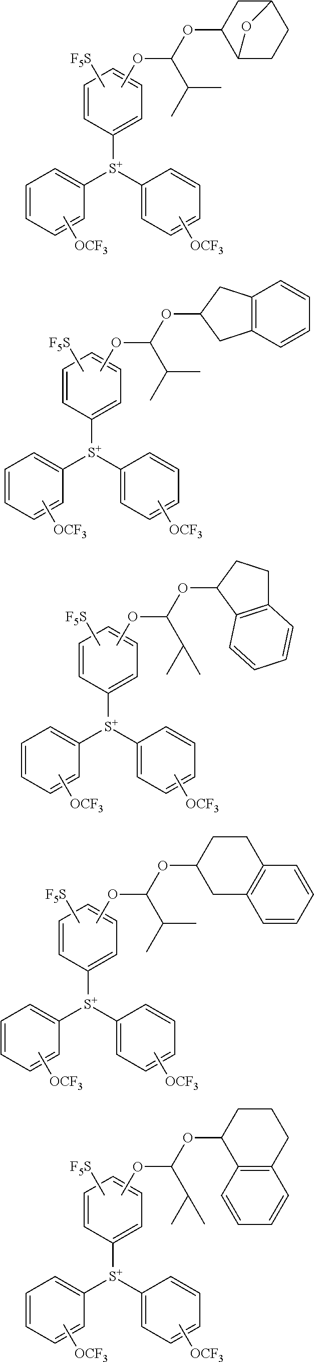

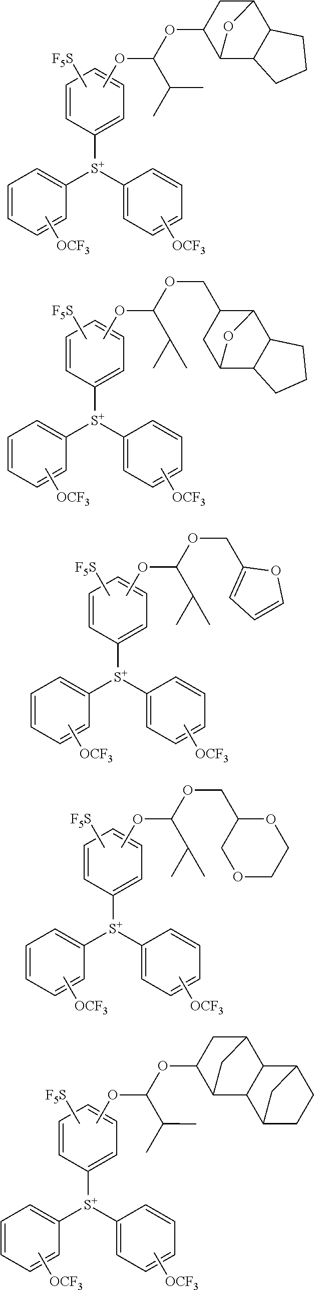

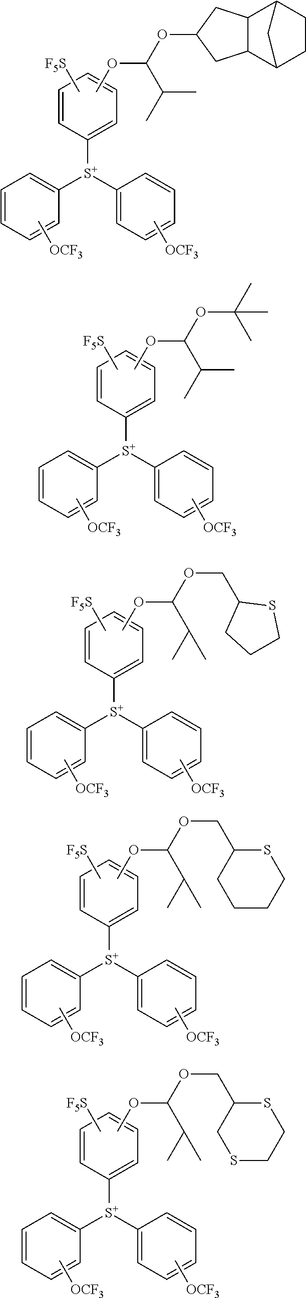

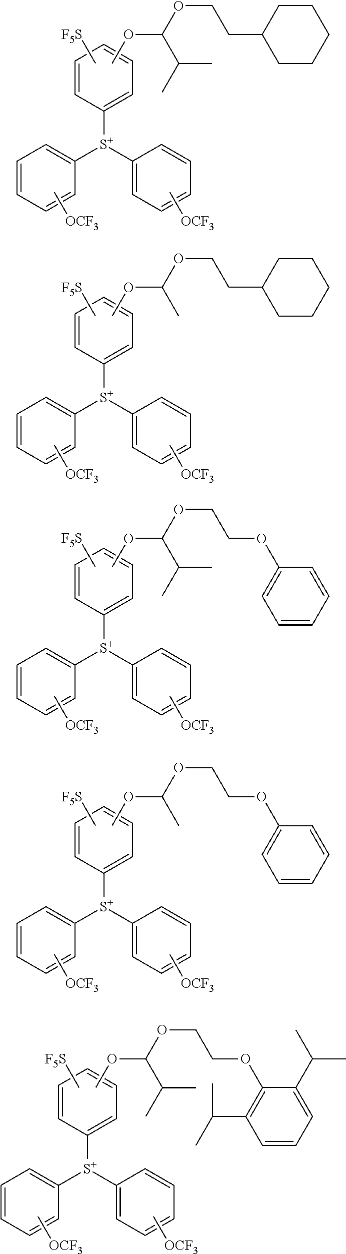

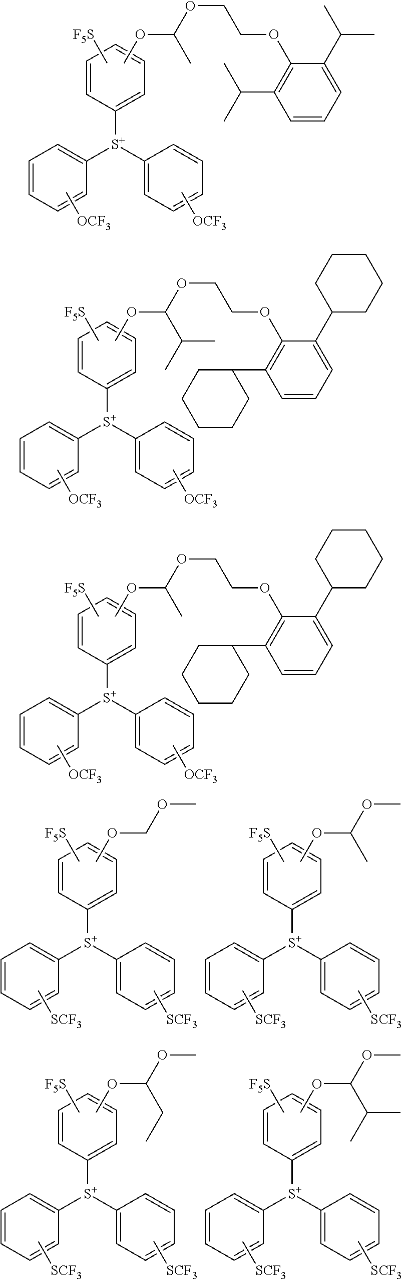

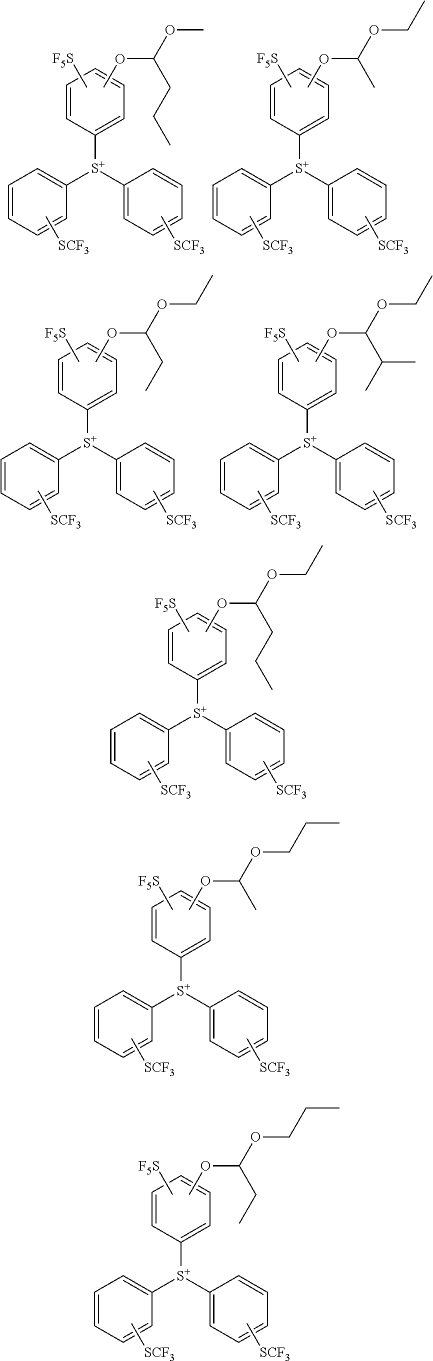

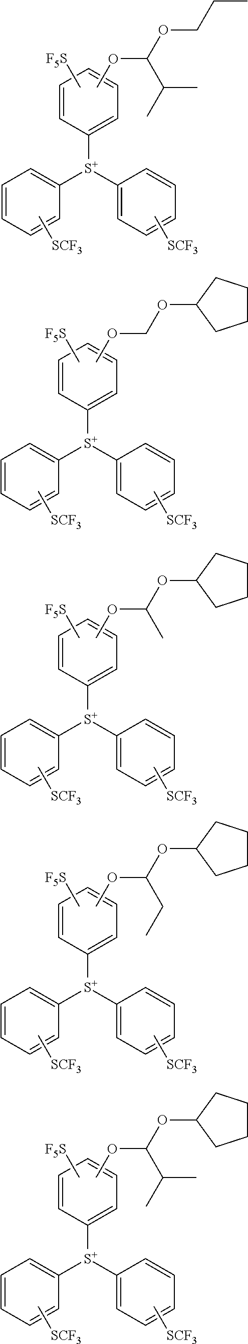

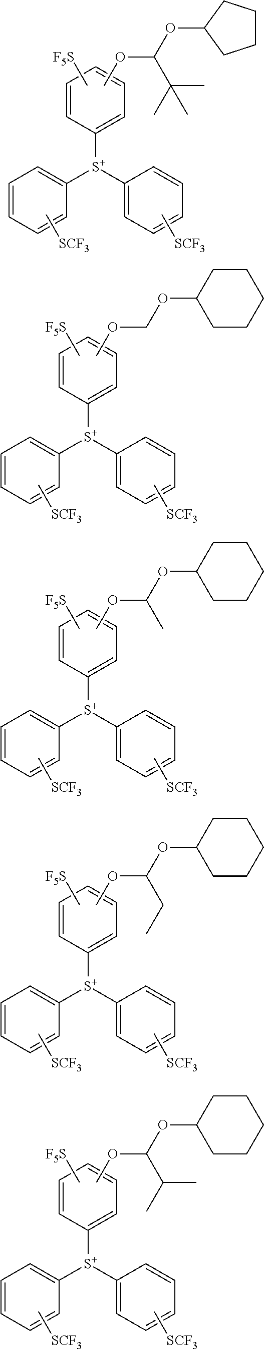

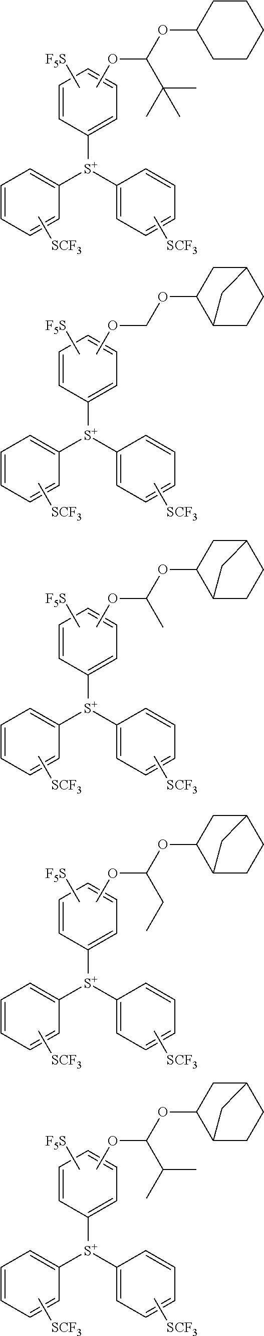

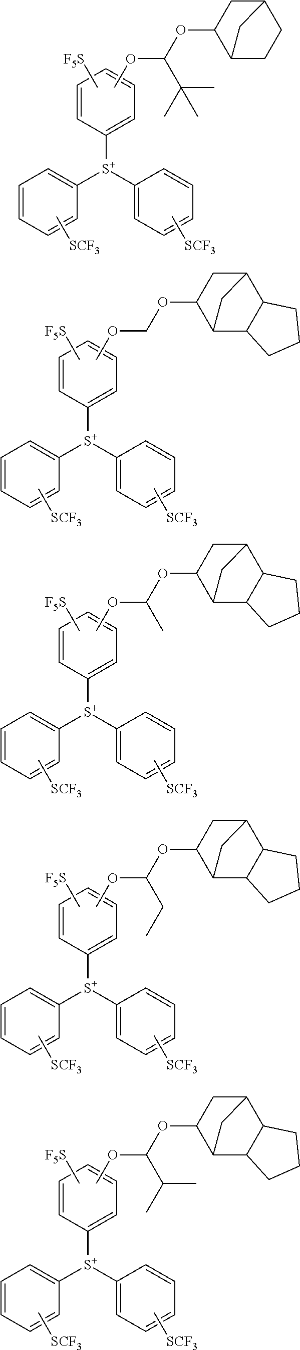

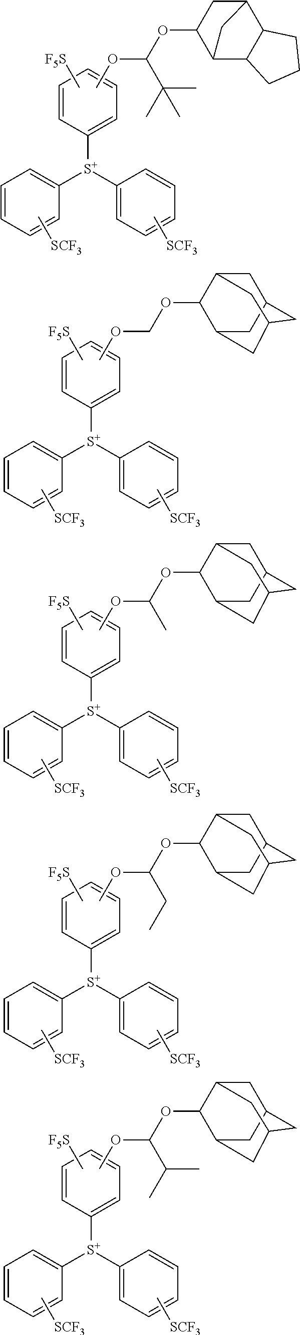

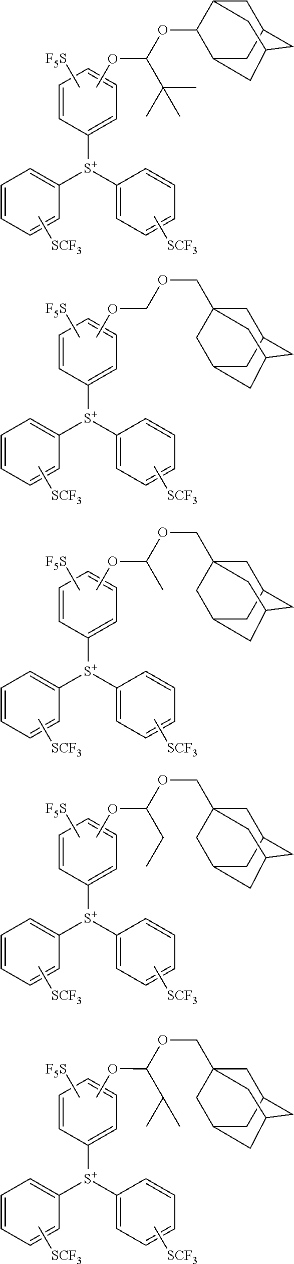

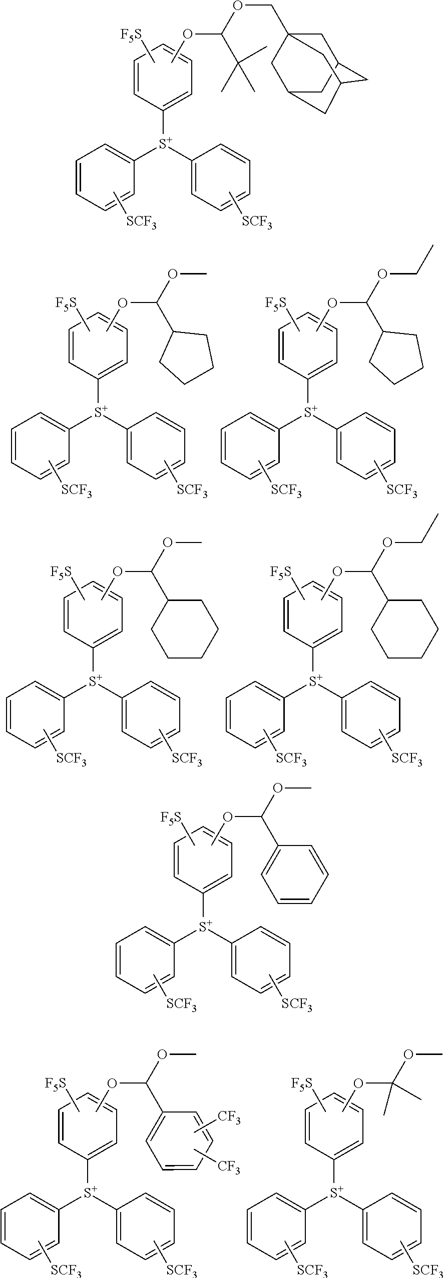

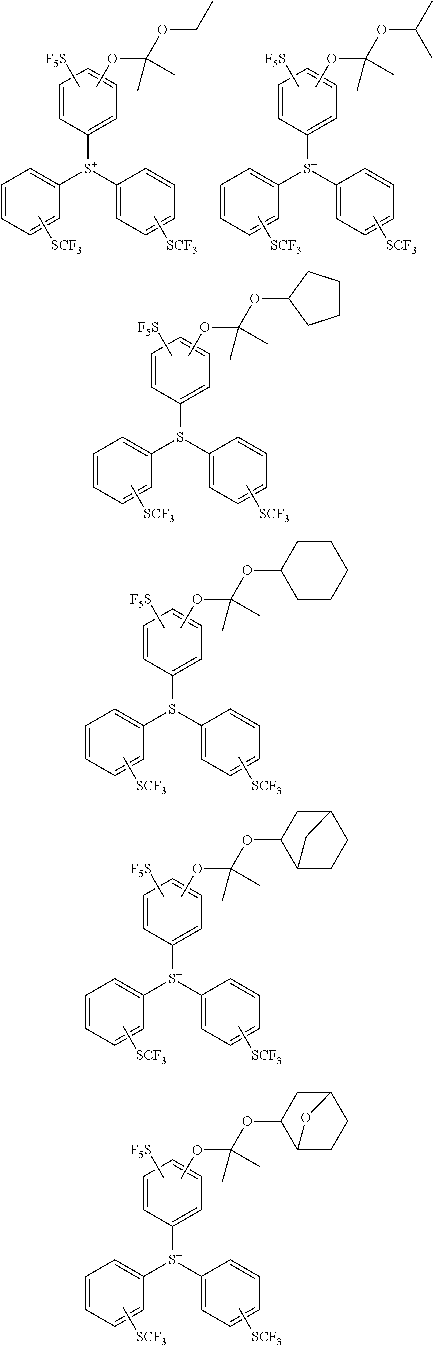

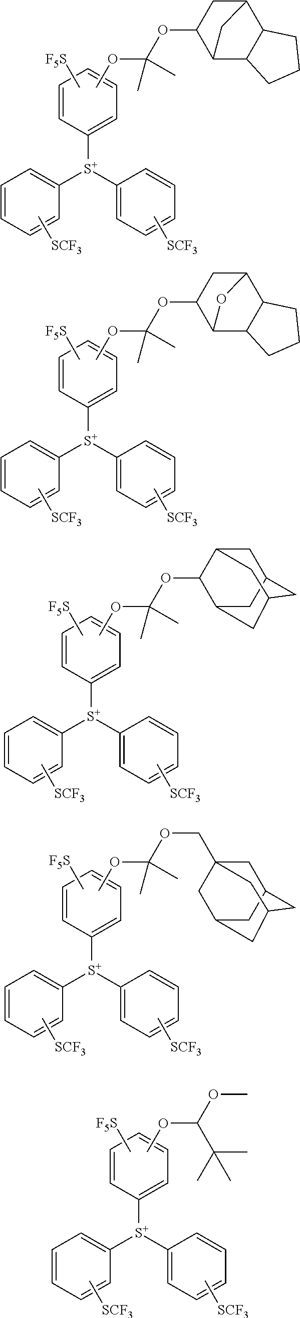

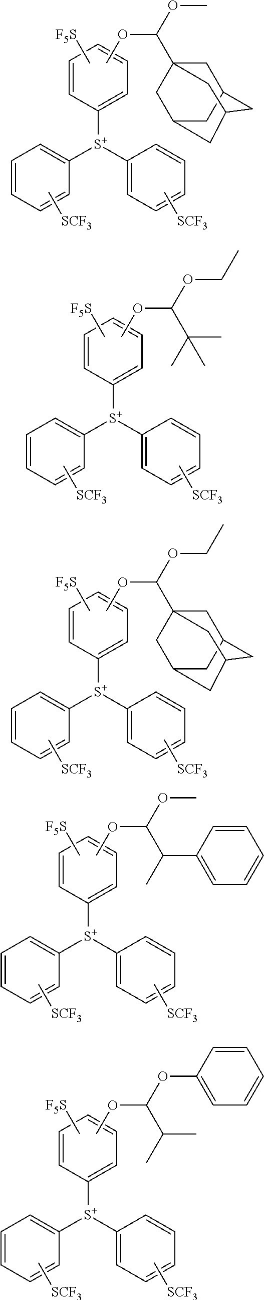

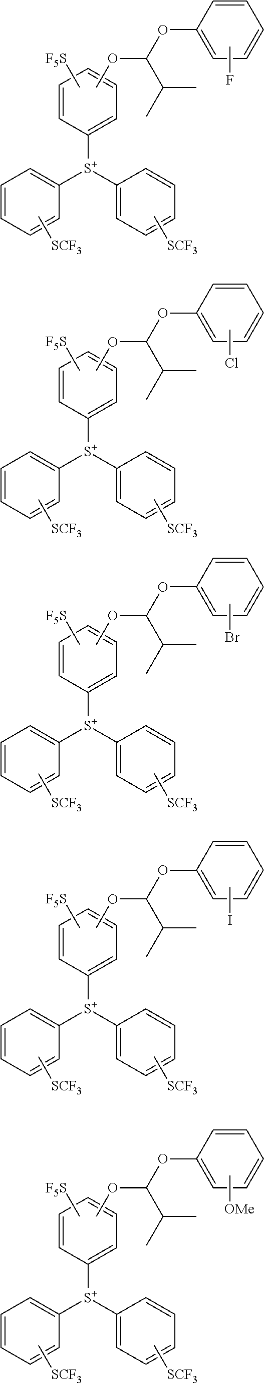

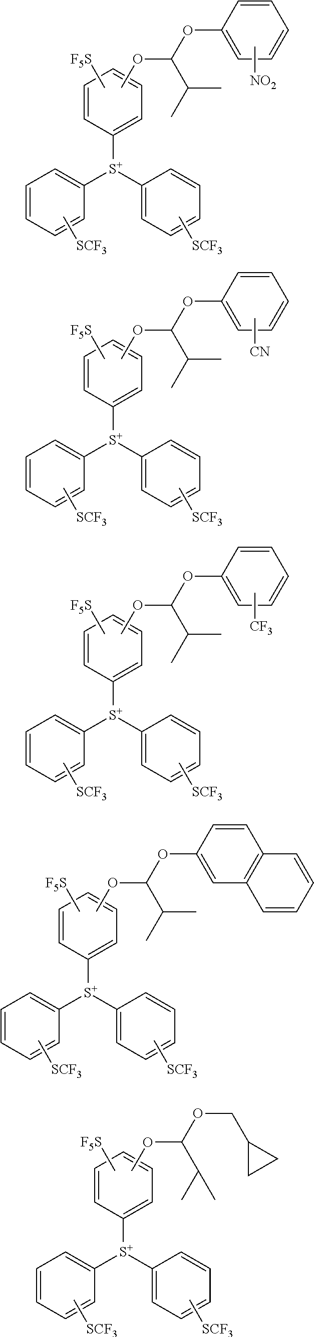

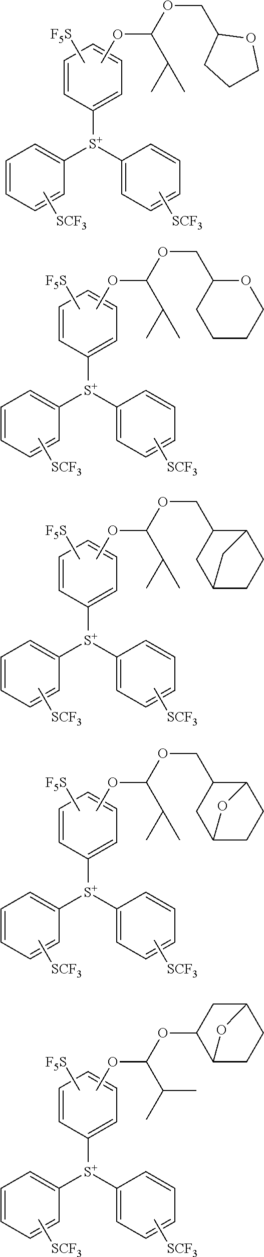

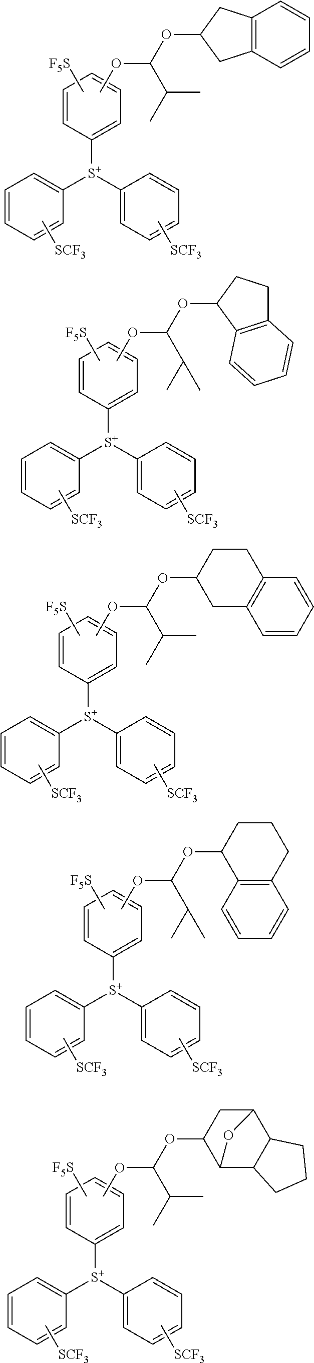

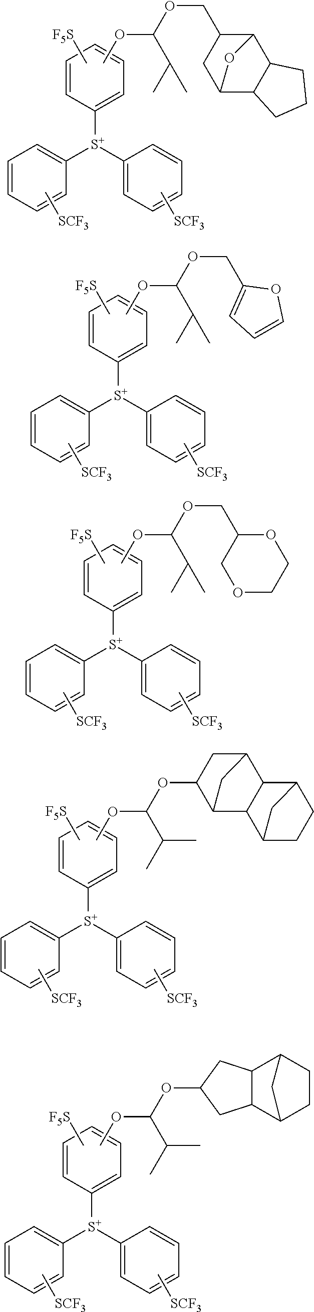

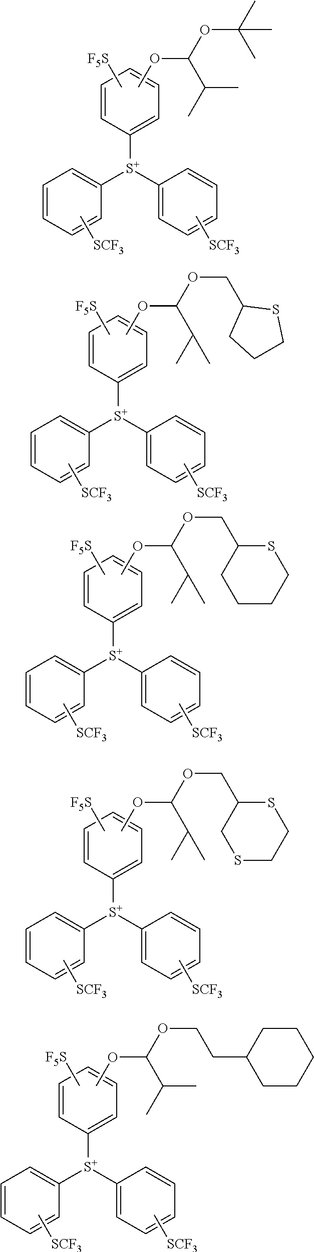

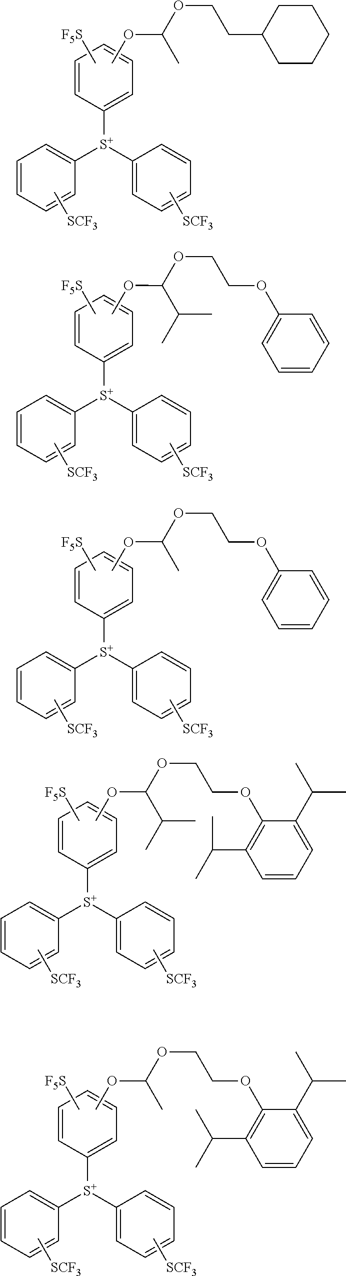

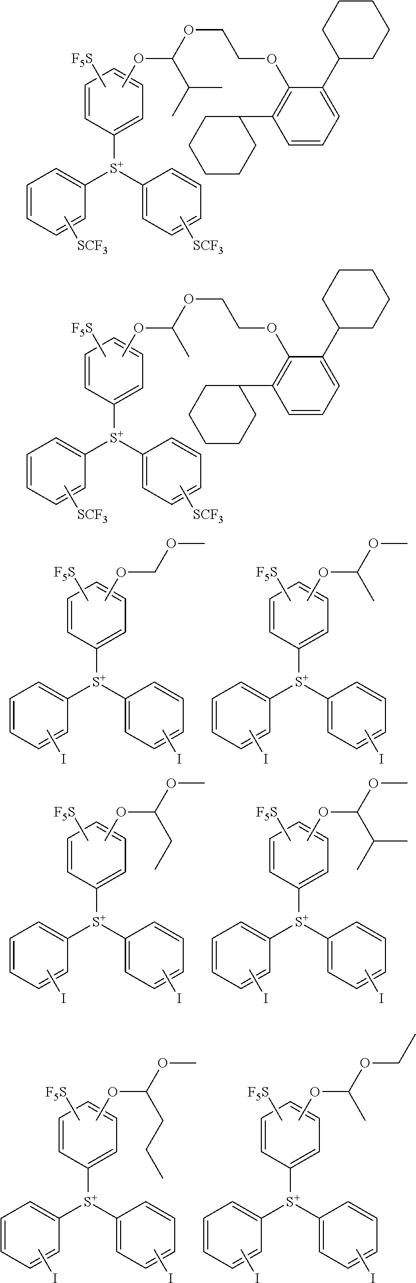

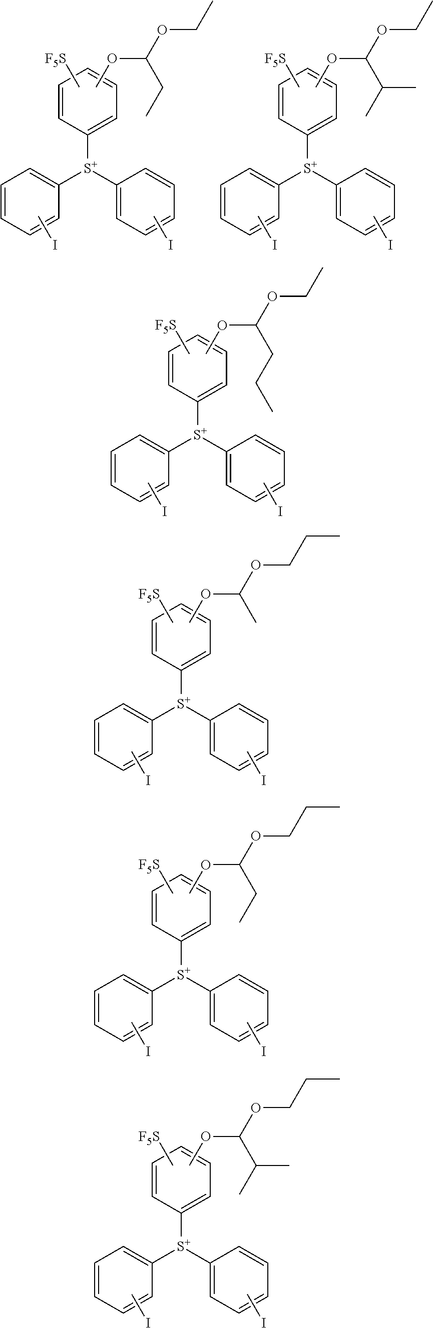

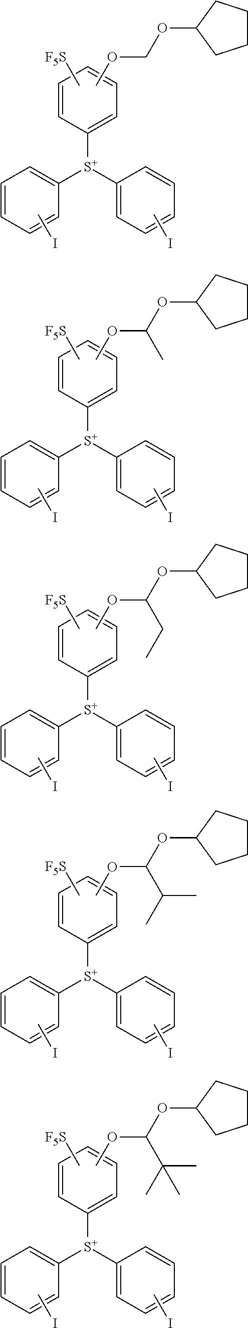

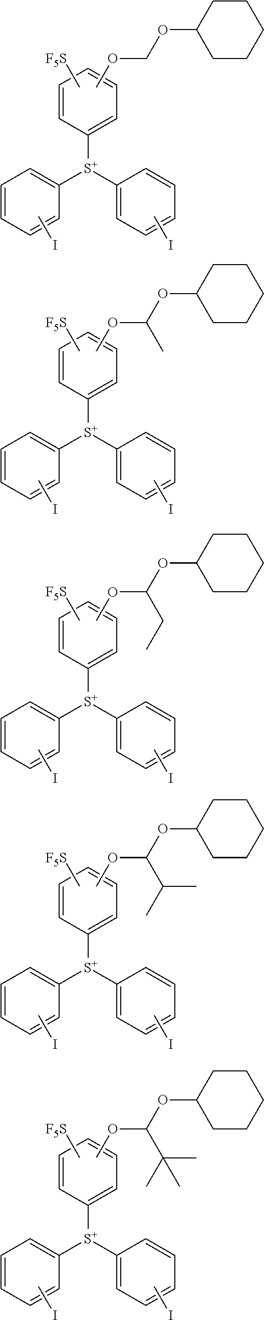

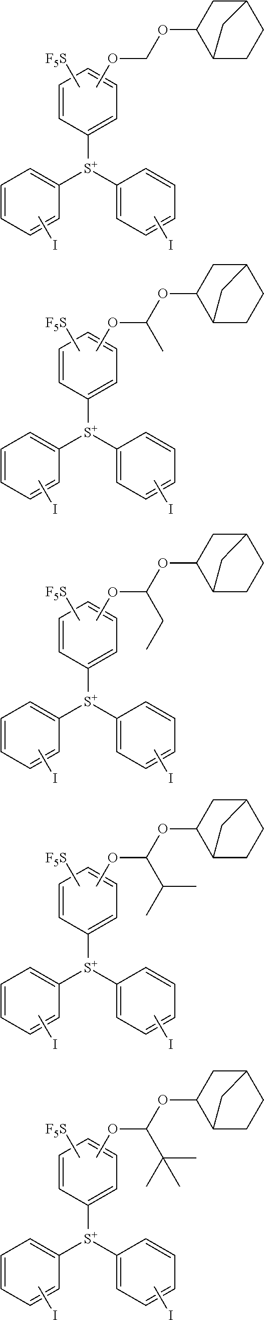

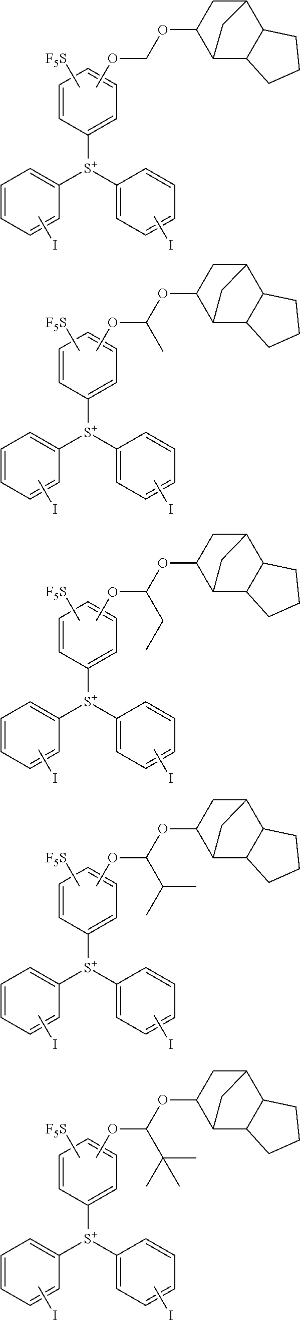

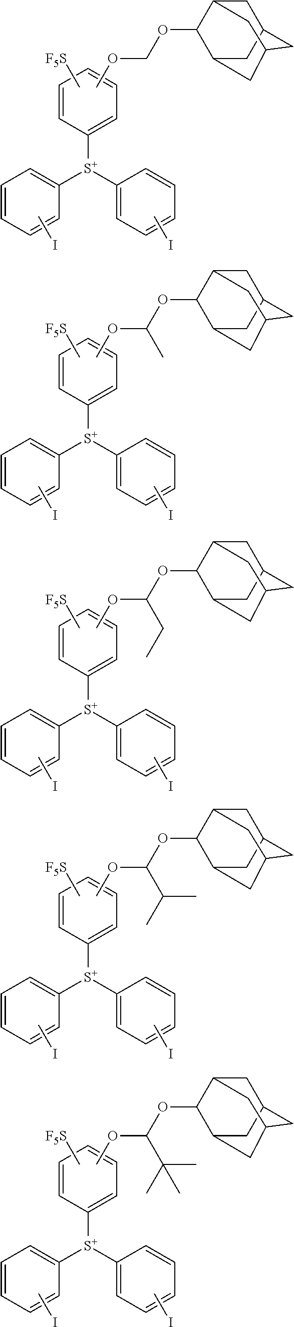

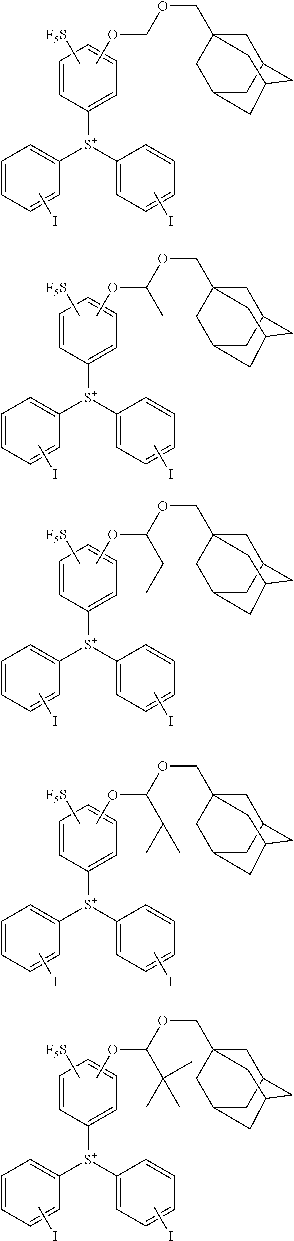

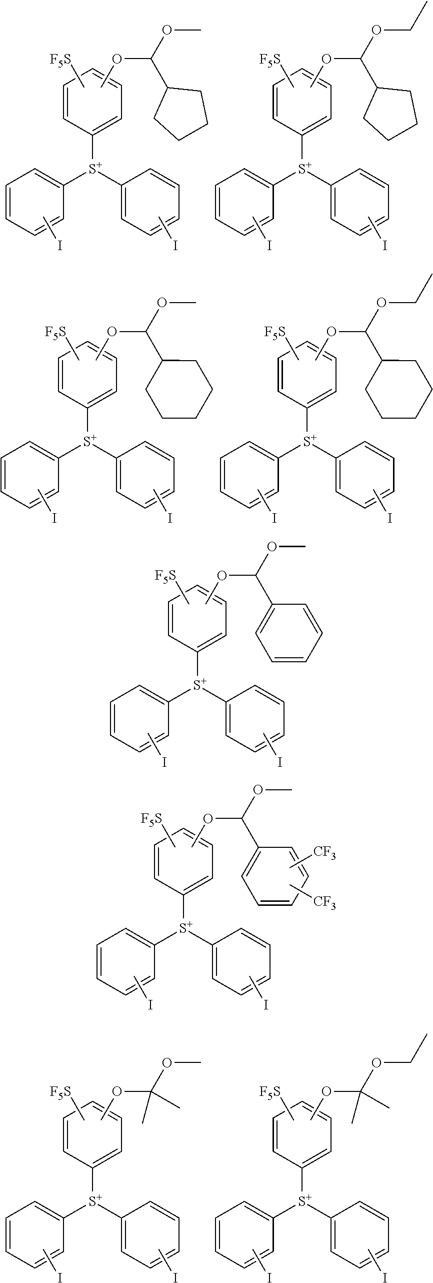

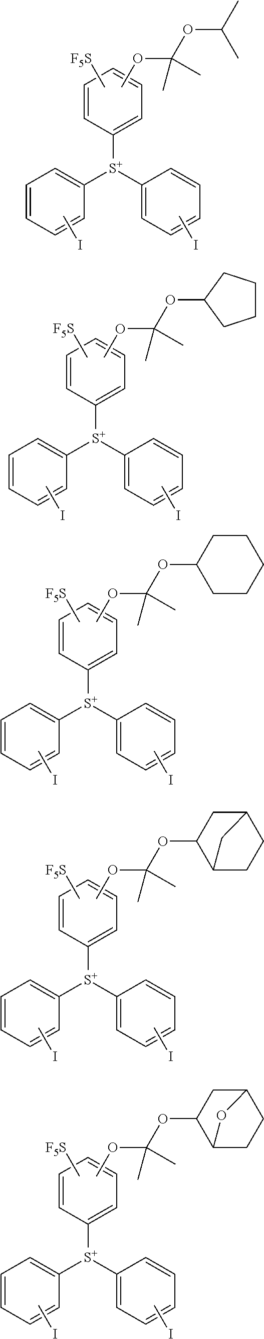

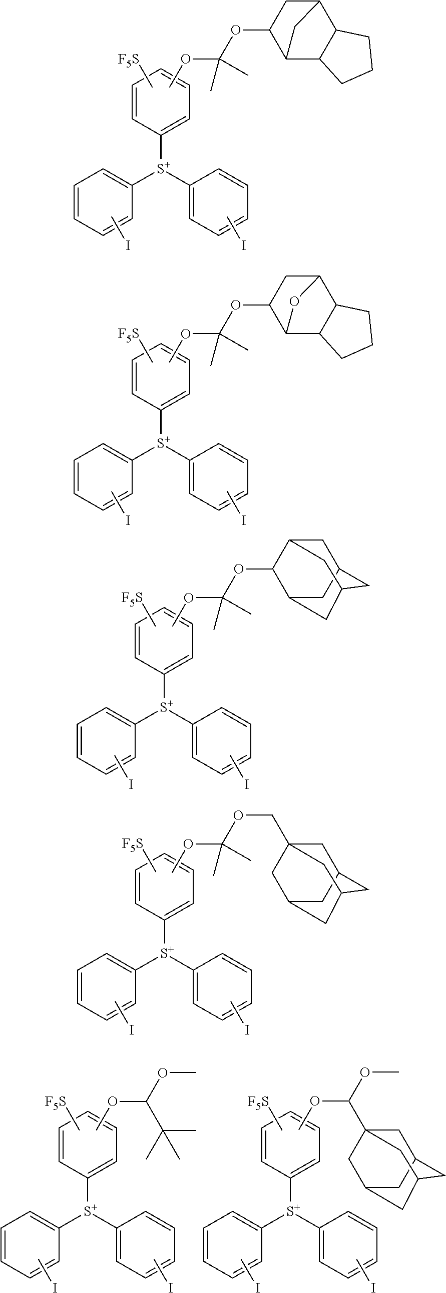

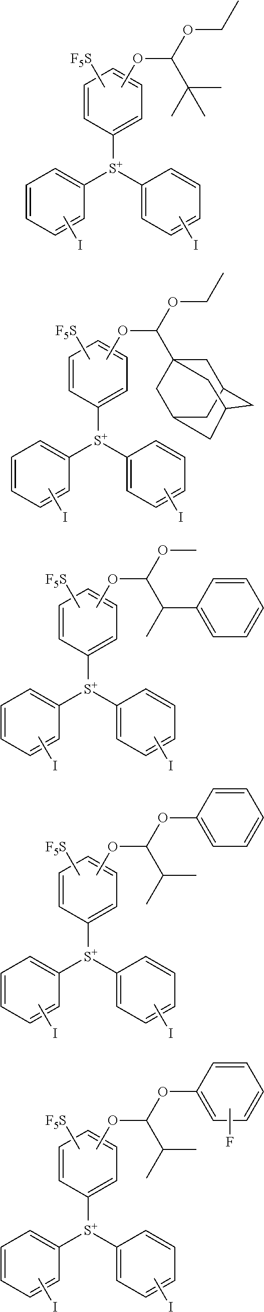

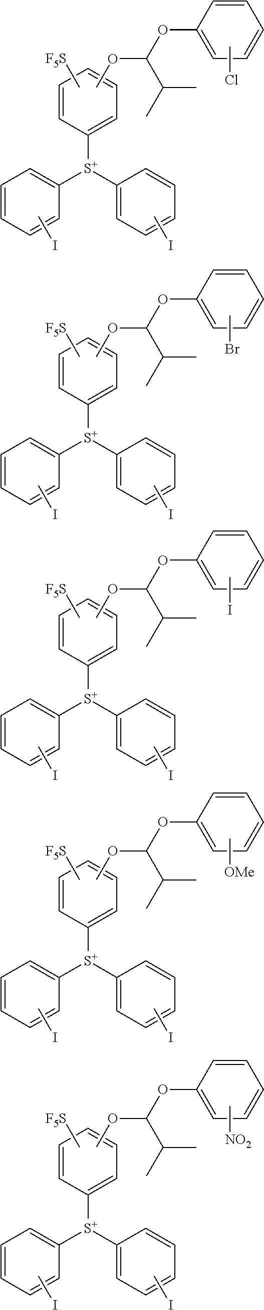

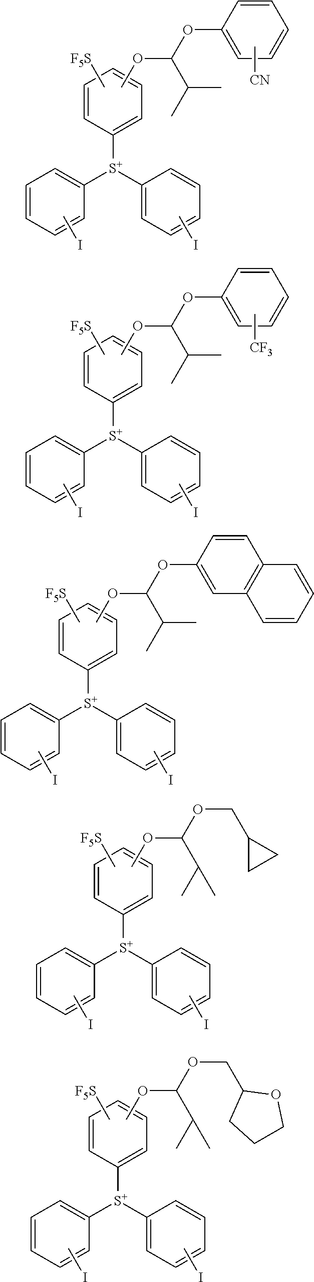

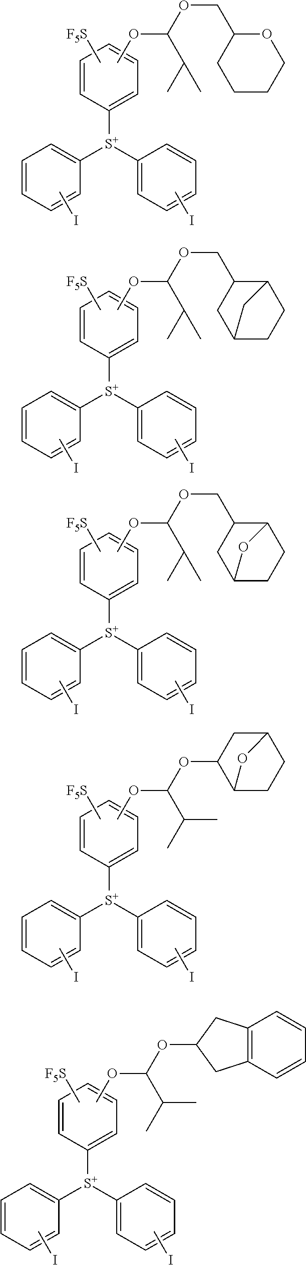

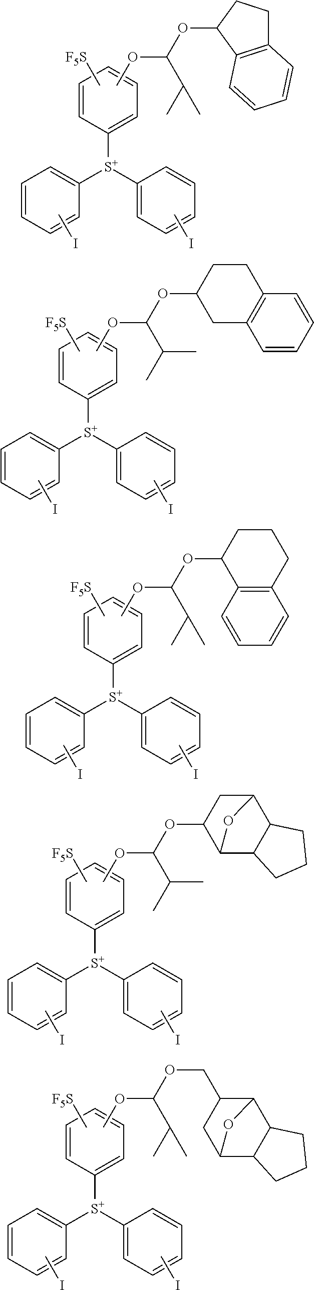

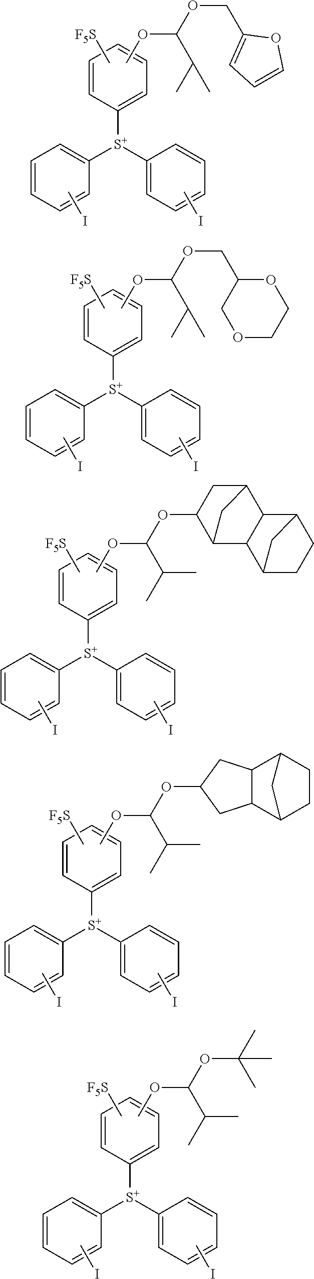

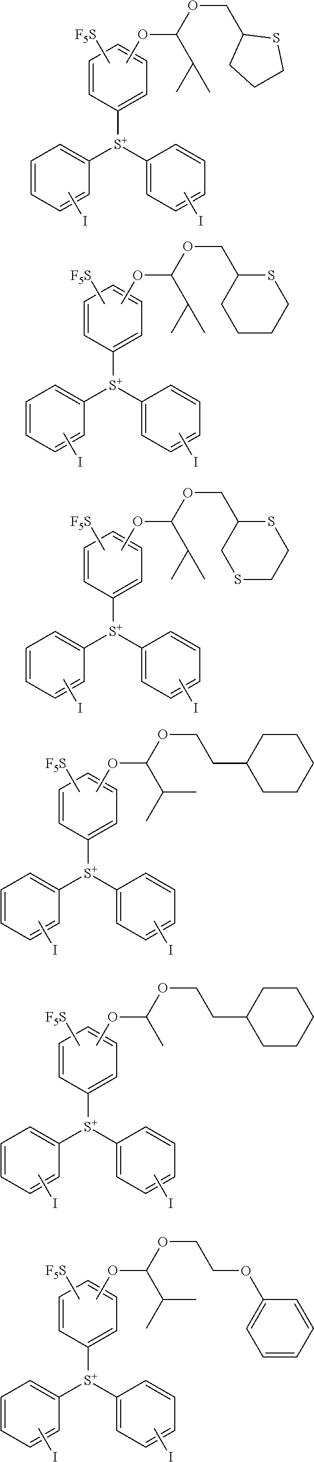

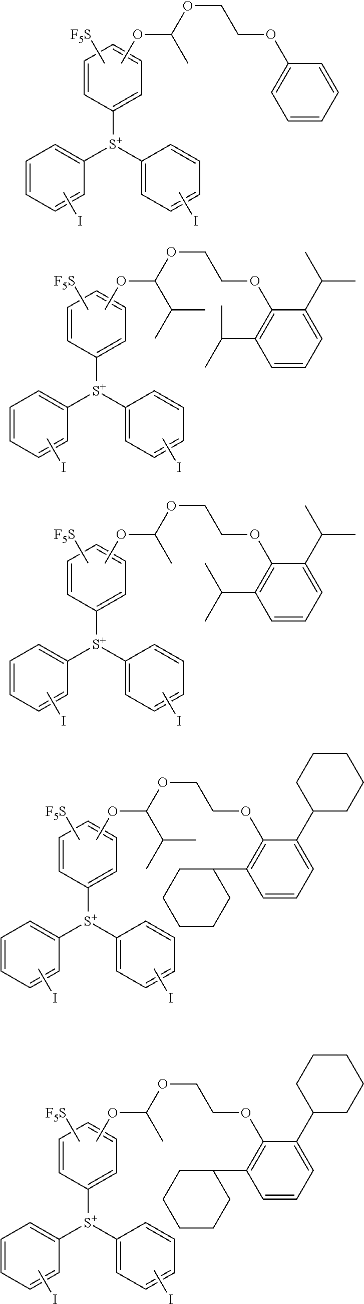

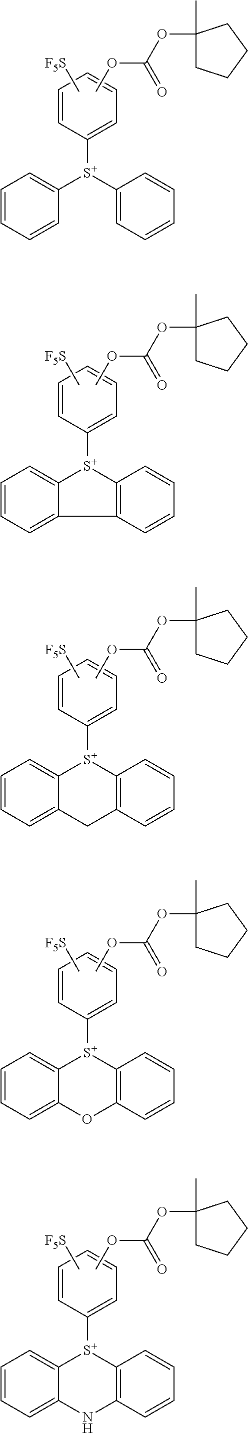

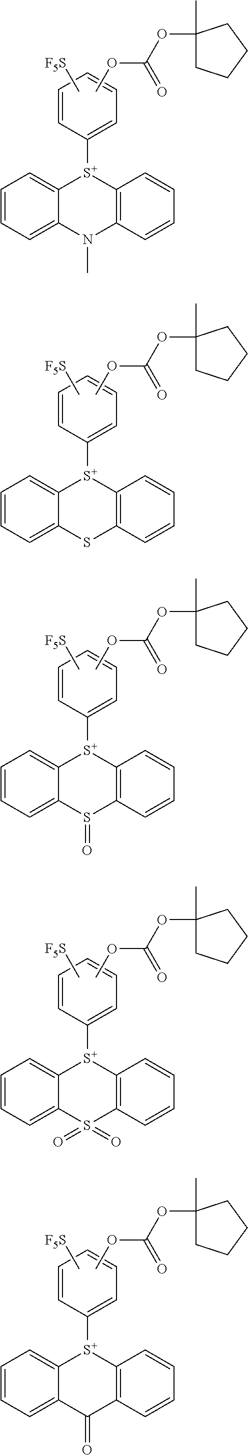

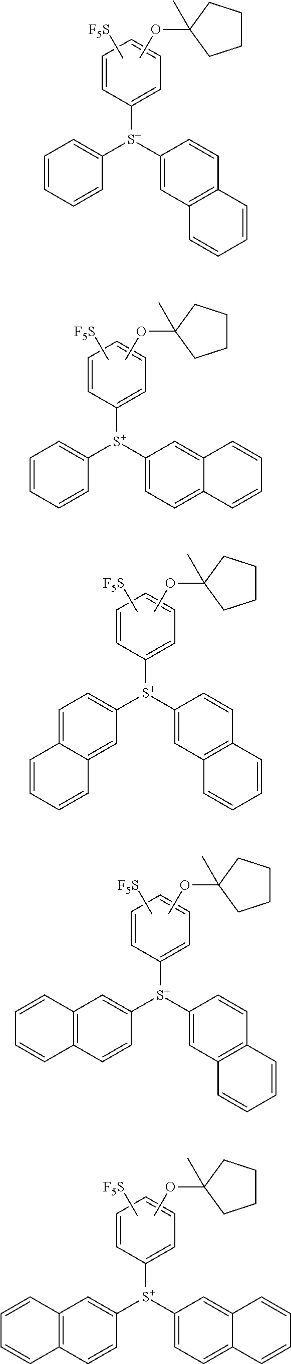

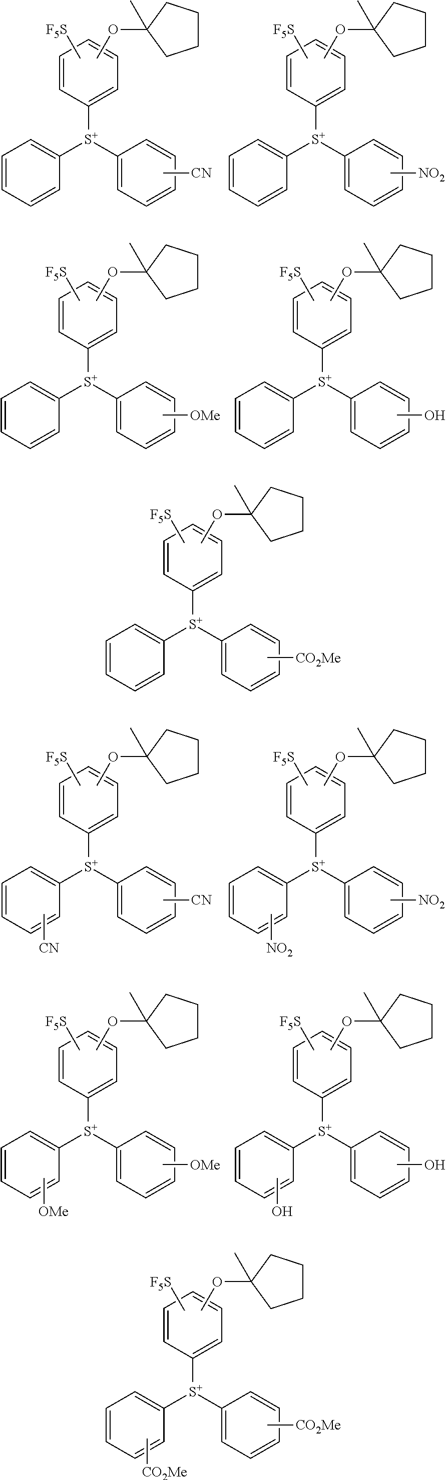

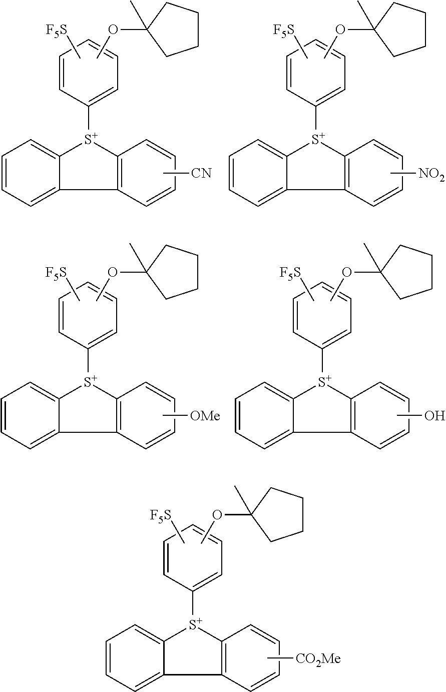

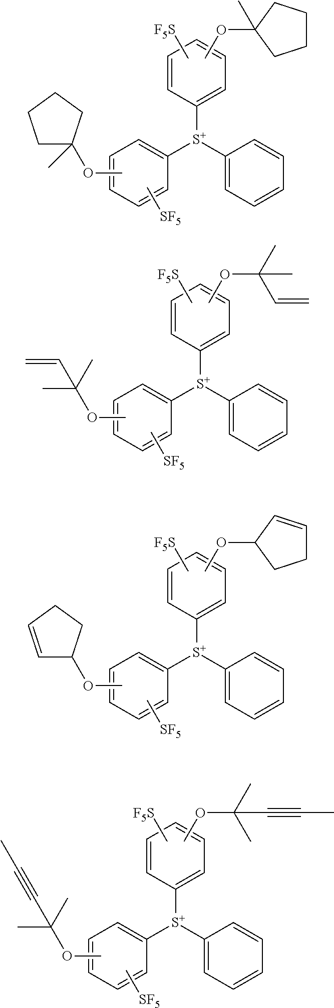

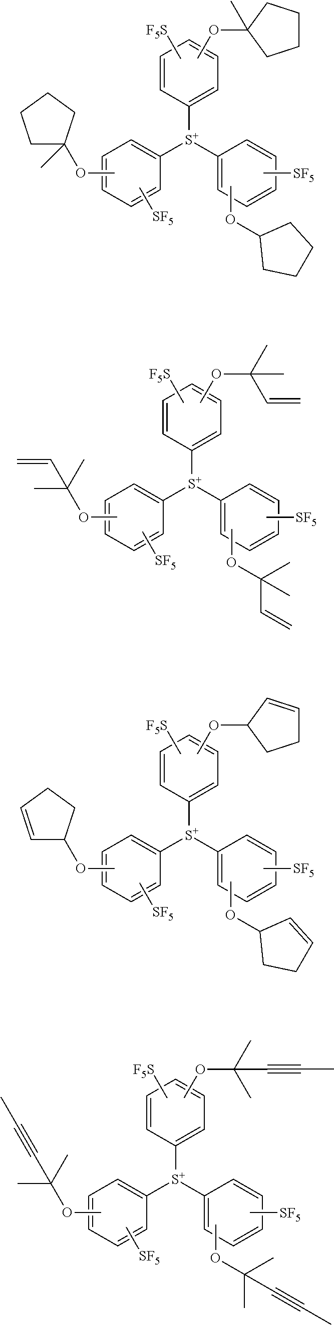

In formulae (1) and (1A), Z⁻ is an aromatic carboxylic acid anion which may have a substituent. The aromatic carboxylic acid anion preferably has the formula (Z).

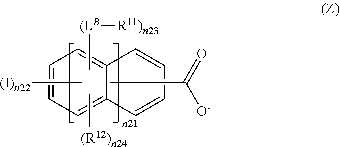

In formula (Z), n21 is 0 or 1. The relevant structure is a benzene ring when n21=0, and a naphthalene ring in case of n21=1. From the aspect of solvent solubility, the benzene ring corresponding to n21=0 is preferred. The subscript n22 is 0, 1, 2, 3 or 4, preferably 0, 1, 2 or 3 from the aspect of reactant availability, and more preferably 1 or 2 from the aspect of solvent solubility. The subscript n23 is 0, 1 or 2, and n24 is 0, 1, 2, 3 or 4. These subscripts meet 2≤n22+n23+n24≤5 when n21-0 and 2≤n22+n23+n24≤7 when n21=1. From the aspect of reactant availability, n24 is preferably 0, 1, 2 or 3, more preferably 0, 1 or 2.

In formula (Z), L^{B} is a single bond, oxygen, sulfur, ester bond or carbonate bond. L^{B} is preferably oxygen when R¹¹ is hydrogen. L^{B} is preferably oxygen, ester bond or carbonate bond, more preferably oxygen or ester bond when R¹¹ is an acid labile group.

In formula (Z), when L^{B} is a single bond, R¹¹ is hydrogen or a C₁-C₂₀ hydrocarbyl group which may contain a heteroatom. The C₁-C₂₀ hydrocarbyl group may be saturated or unsaturated and straight, branched or cyclic. Examples of the hydrocarbyl group include C₁-C₂₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, n-decyl, n-undecyl and n-dodecyl; C₃-C₂₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, 1-propenyl, 2-propenyl, butenyl, pentenyl and hexenyl; C₂-C₂₀ alkynyl groups such as ethynyl, propynyl, butynyl, pentynyl and hexynyl; C₃-C₂₀ cyclic unsaturated aliphatic hydrocarbyl groups such as cyclopentenyl and cyclohexenyl; C₆-C₂₀ aryl groups such as phenyl, naphthyl and indanyl; C₇-C₂₀ aralkyl groups such as benzyl, 1-phenylethyl and 2-phenylethyl, and combinations thereof. The hydrocarbyl group may contain a heteroatom such as halogen, oxygen or sulfur.

When L^{B} is oxygen, sulfur, ester bond or carbonate bond, R¹¹ is hydrogen, a C₁-C₂₀ hydrocarbyl group (exclusive of acid labile group) which may contain a heteroatom, or acid labile group. Examples of the hydrocarbyl group are as exemplified above, with acid labile groups being excluded.

Typical of the acid labile group are groups having the following formulae (AL-3) and (AL-4).

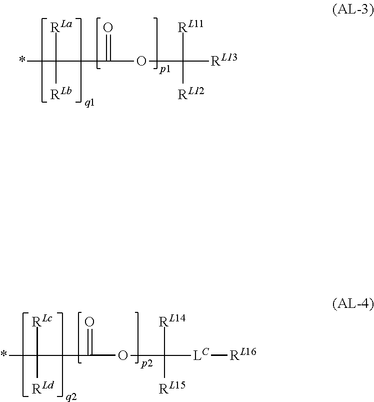

The asterisk (*) designates a point of attachment to the adjacent oxygen or sulfur.

In formulae (AL-3) and (AL-4), p1 and p2 are each independently 0 or 1, q1 and q2 are each independently 0, 1, 2, 3 or 4. When L^{B} is oxygen and a carbonate bond is formed with the oxygen atom, p1 or p2 is 1. Otherwise, p1 or p2 is preferably 0. When p1=1, q1 is preferably 1 or 2, and when p1=0, q1 is 0. When p2=1, q2 is preferably 1 or 2, and when p2=0, q2 is 0.

In formula (AL-3), R^{L11}, R^{L12} and R^{L13} are each independently a C₁-C₁₂ hydrocarbyl group in which some —CH₂— may be replaced by —O— or —S—. When the hydrocarbyl group contains an aromatic ring, some or all of the hydrogen atoms in the aromatic ring may be substituted by halogen, cyano moiety, nitro moiety, optionally halogenated C₁-C₄ alkyl moiety, or optionally halogenated C₁-C₄ alkoxy moiety.

The C₁-C₁₂ hydrocarbyl group represented by R^{L11}, R^{L12} and R^{L13} may be saturated or unsaturated and straight, branched or cyclic. Examples thereof include C₁-C₁₂ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, n-decyl, n-undecyl, and n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₁₂ alkenyl groups such as vinyl, 1-propenyl, 2-propenyl, butenyl, pentenyl, and hexenyl; C₂-C₁₂ alkynyl groups such as ethynyl, propynyl, butynyl, pentynyl and hexynyl; C₃-C₁₂ cyclic unsaturated aliphatic hydrocarbyl groups such as cyclopentenyl and cyclohexenyl; C₆-C₁₂ aryl groups such as phenyl, naphthyl and indanyl; C₇-C₁₂ aralkyl groups such as benzyl, 1-phenylethyl, and 2-phenylethyl, and combinations thereof.

R^{L11} and R^{L12} may bond together to form a ring with the carbon atom to which they are attached, and some —CH₂— in the ring may be replaced by —O— or —S—. Exemplary rings include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}.0^{2,7}]dodecane rings as well as these rings in which some —CH₂— is replaced by —O— or —S—.

In formula (AL-4), R^{L14} and R^{L15} are each independently hydrogen or a C₁-C₁₀ hydrocarbyl group. The hydrocarbyl group represented by R^{L14} and R^{L15} may be saturated or unsaturated and straight, branched or cyclic. Examples thereof include those exemplified above for the hydrocarbyl group represented by R^{L11}, R^{L12} and R^{L13}, but of 1 to 10 carbon atoms.

In formula (AL-4), R^{L16} is a C₁-C₂₀ hydrocarbyl group in which some —CH₂— may be replaced by —O— or —S—. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, propenyl, butenyl, pentenyl and hexenyl; C₂-C₂₀ alkynyl groups such as ethynyl, propynyl, butynyl, pentynyl and hexynyl; C₃-C₂₀ cyclic unsaturated aliphatic hydrocarbyl groups such as cyclopentenyl, cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl, and combinations thereof. Also, R^{L15} and R^{L16} may bond together to form a C₃-C₂₀ heterocyclic group with the carbon atom and L^{C} to which they are attached, and some —CH₂— in the heterocyclic group may be replaced by —O— or —S—.

In formula (AL-4), L^{C} is —O— or —S—

In formulae (AL-3) and (AL-4), R^{La} to R^{Ld} are each independently hydrogen or a C₁-C₂₀ hydrocarbyl group which may contain a heteroatom. The hydrocarbyl group may be saturated or unsaturated and straight, branched or cyclic. Examples thereof include those exemplified above for the hydrocarbyl group R¹¹, but are not limited thereto.

Examples of the acid labile group having formula (AL-3) are as exemplified above for the acid labile group having formula (AL-1), but not limited thereto.

Examples of the acid labile group having formula (AL-4) are as exemplified above for the acid labile group having formula (AL-2), but not limited thereto.

In formula (Z), R¹² is halogen exclusive of iodine, nitro, cyano, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen exclusive of iodine include fluorine, chlorine and bromine, with fluorine being preferred. The C₁-C₂₀ hydrocarbyl group may be saturated or unsaturated and straight, branched or cyclic. Examples thereof include those exemplified above for the hydrocarbyl group R¹¹. The hydrocarbyl group may contain a heteroatom such as halogen, oxygen or sulfur.

In formula (Z), when n24 is 2, 3 or 4, a plurality of R¹² may bond together to form a ring structure with the carbon atoms on the aromatic ring to which they are attached. Some —CH₂— in the ring may be replaced by —O— or —S—. Exemplary rings include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}.0^{2,7}]dodecane rings as well as these rings in which some —CH₂— is replaced by —O— or —S—.

The aromatic carboxylic acid anion having formula (Z) preferably has the formula (Z1):

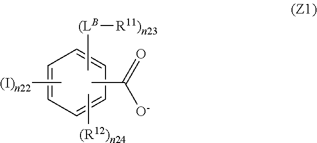

wherein n22 to n24, L^{A}, R¹¹ and R¹² are as defined above.

Examples of the aromatic carboxylic acid anion having formula (Z) are shown below, but not limited thereto.

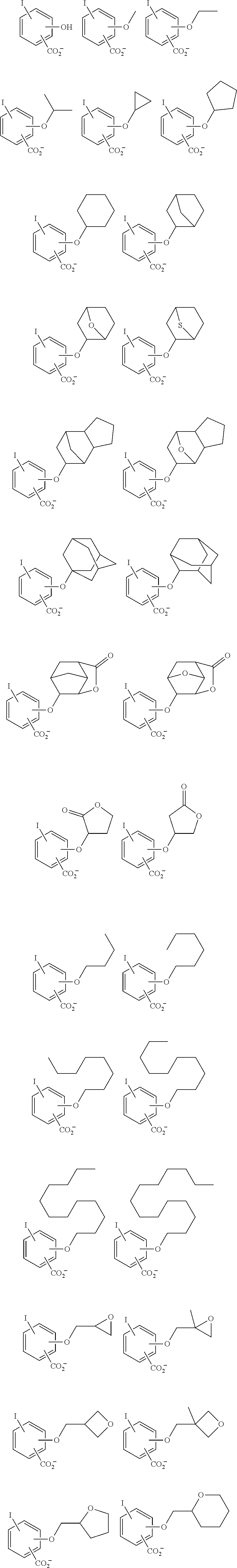

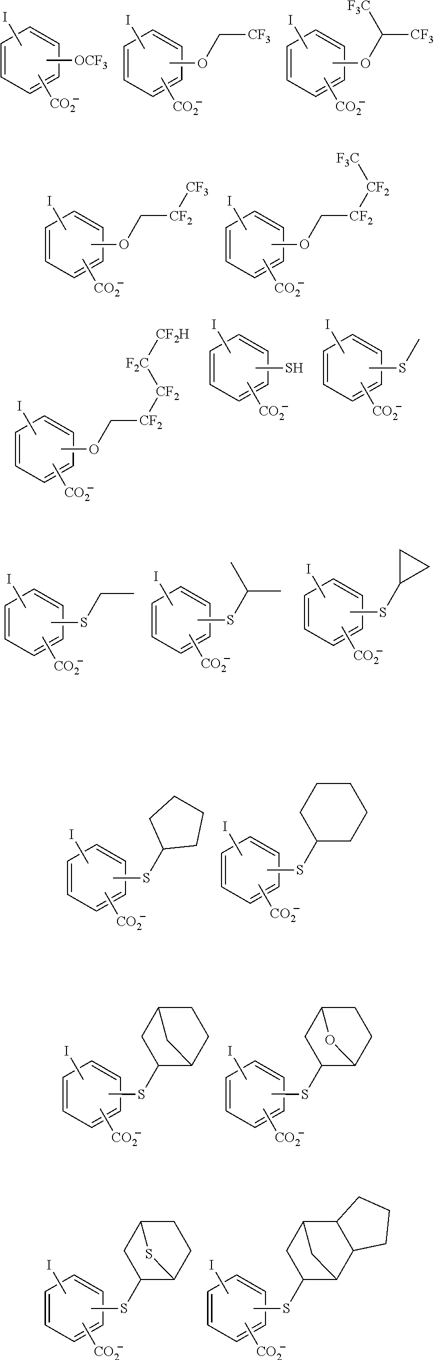

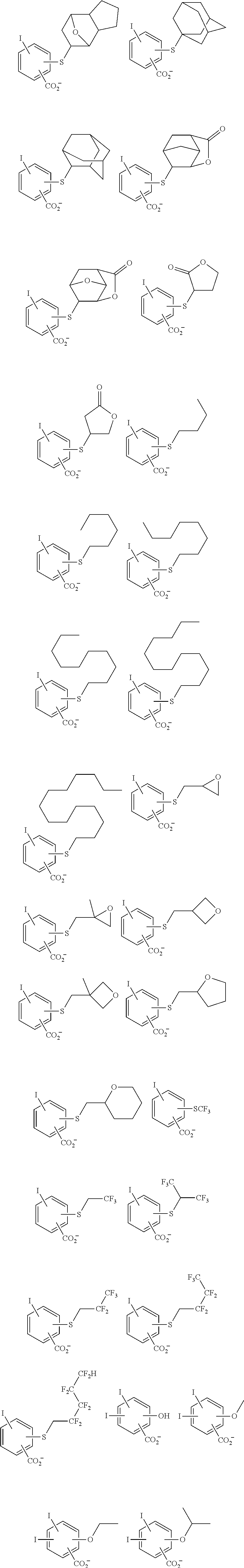

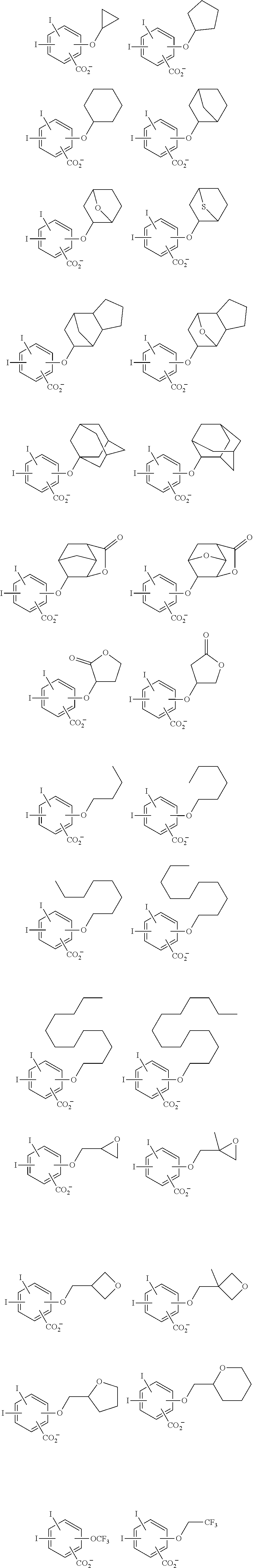

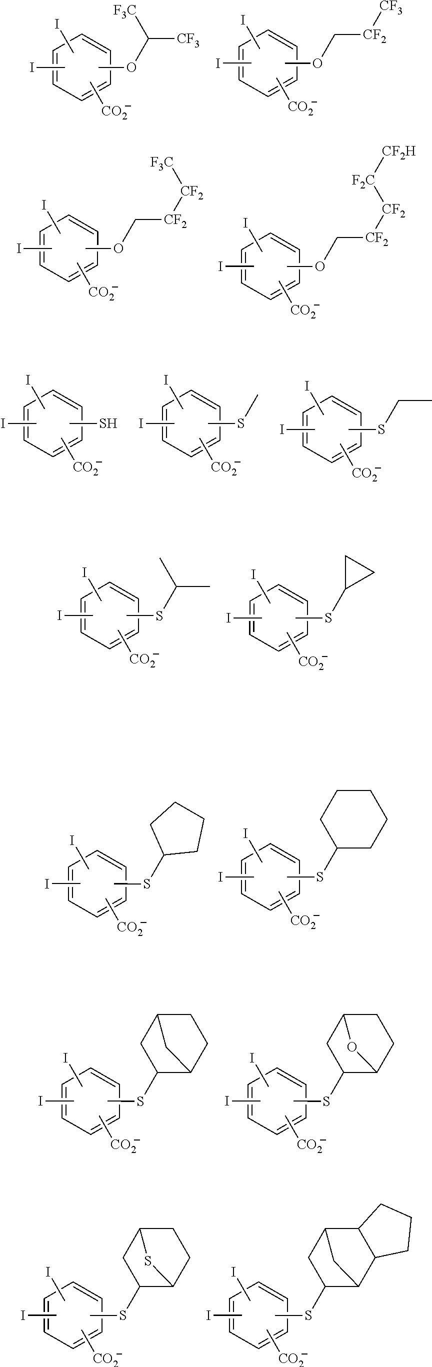

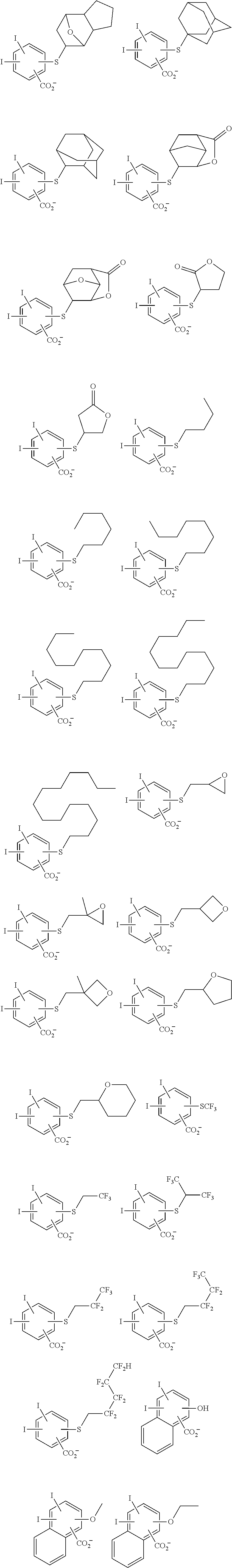

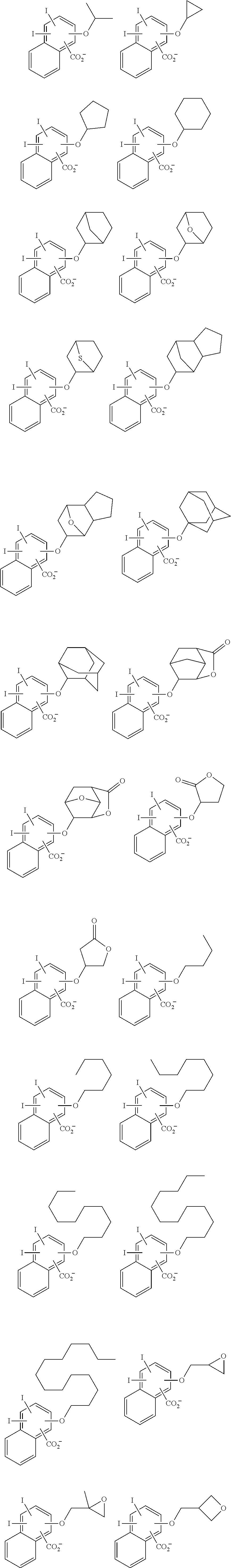

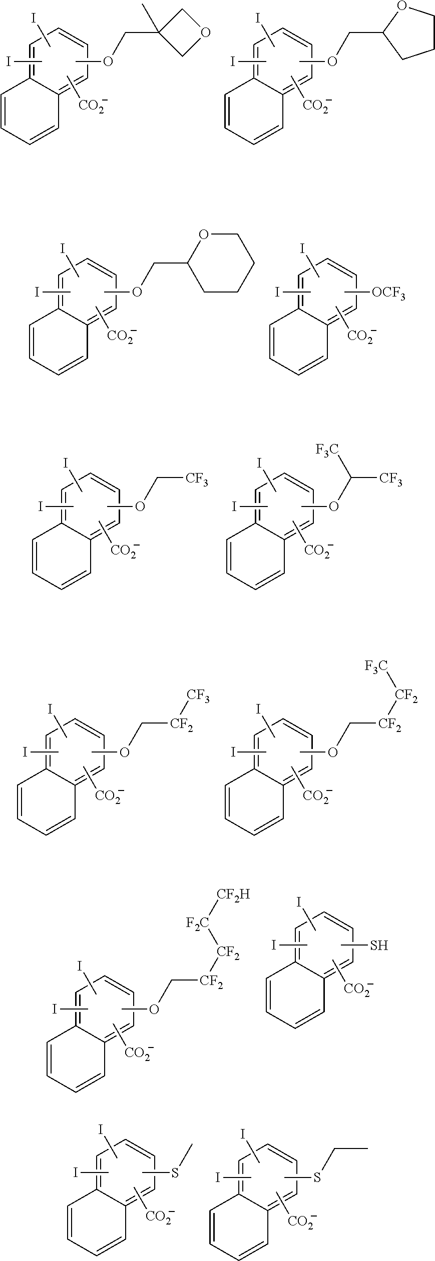

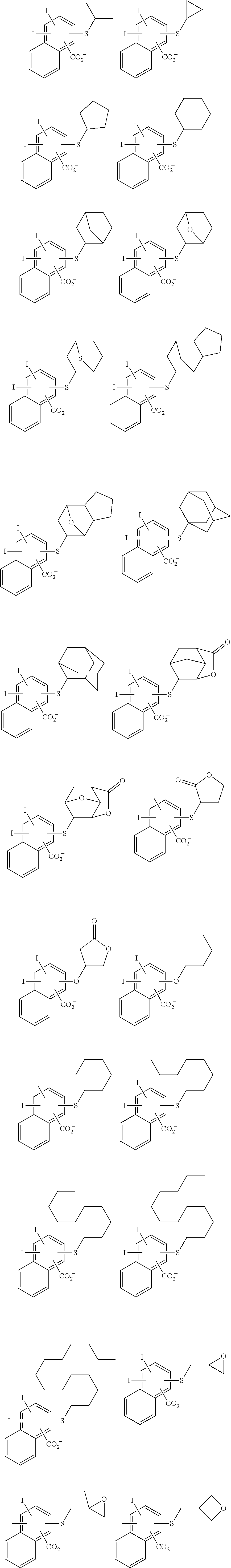

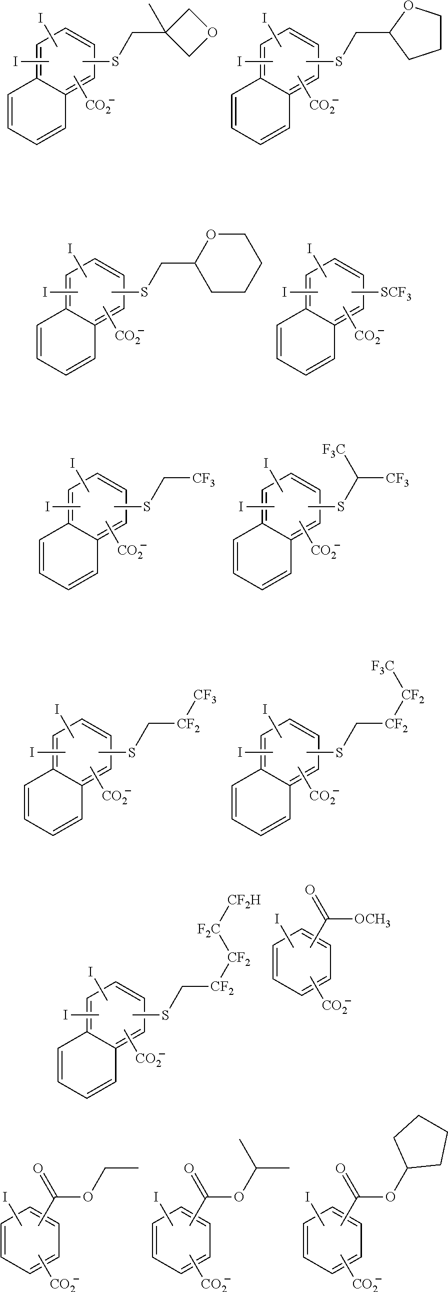

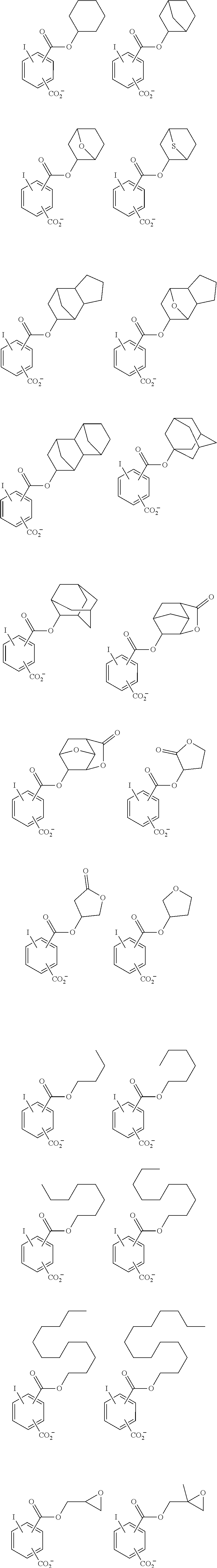

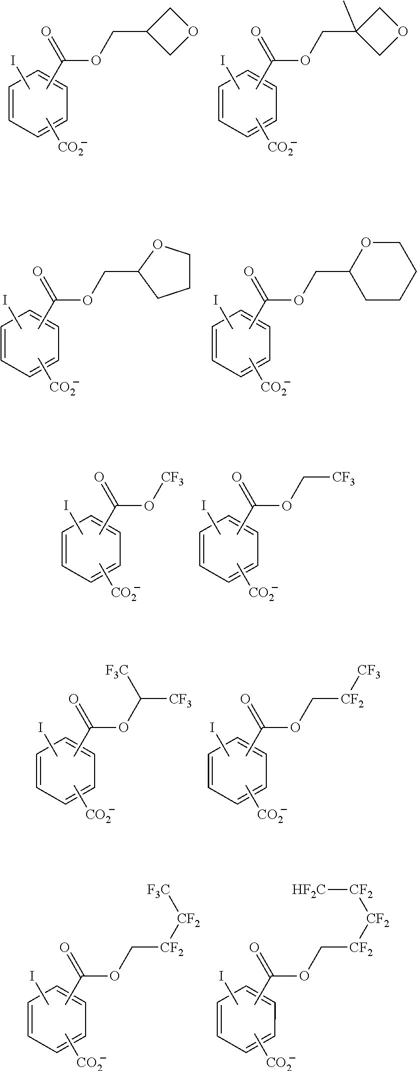

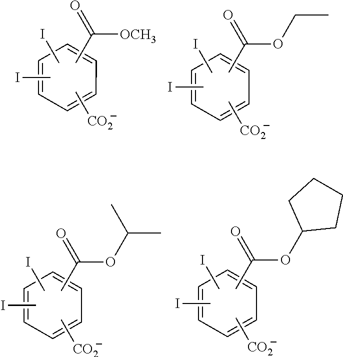

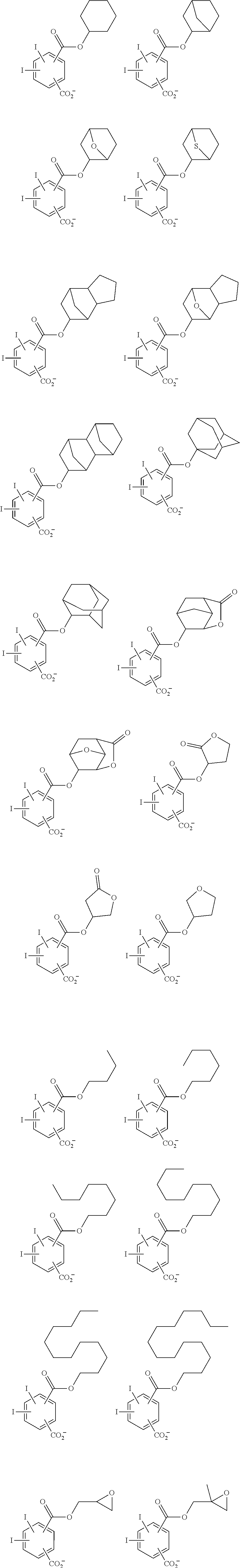

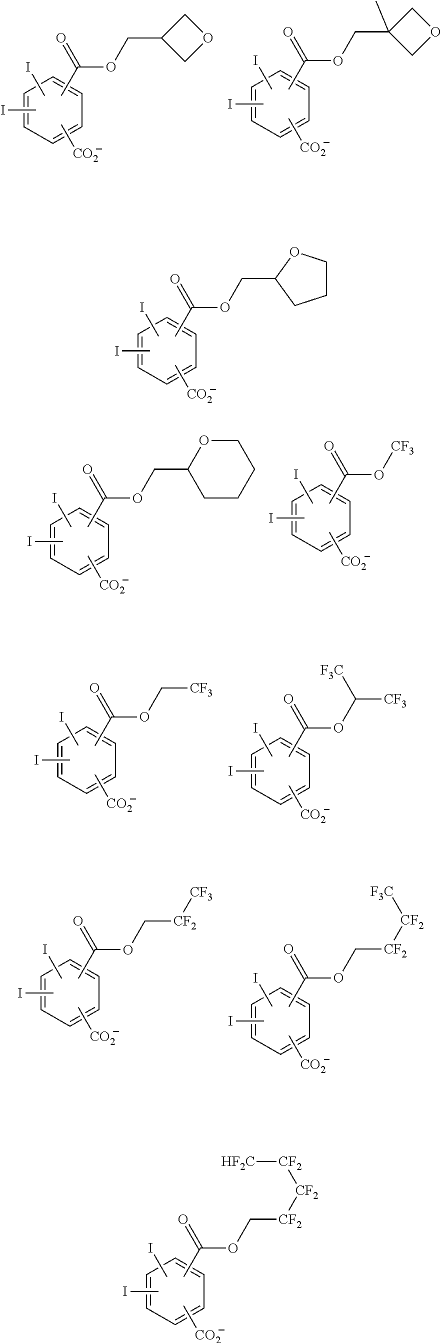

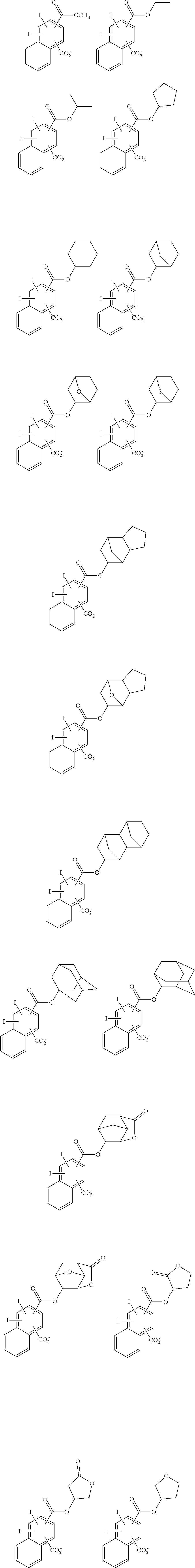

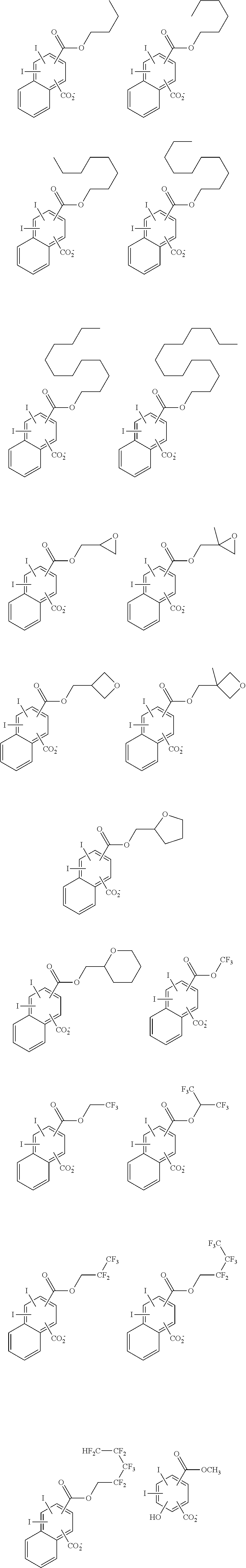

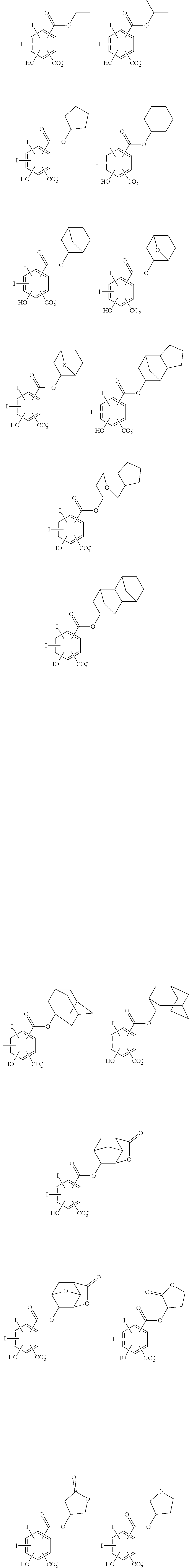

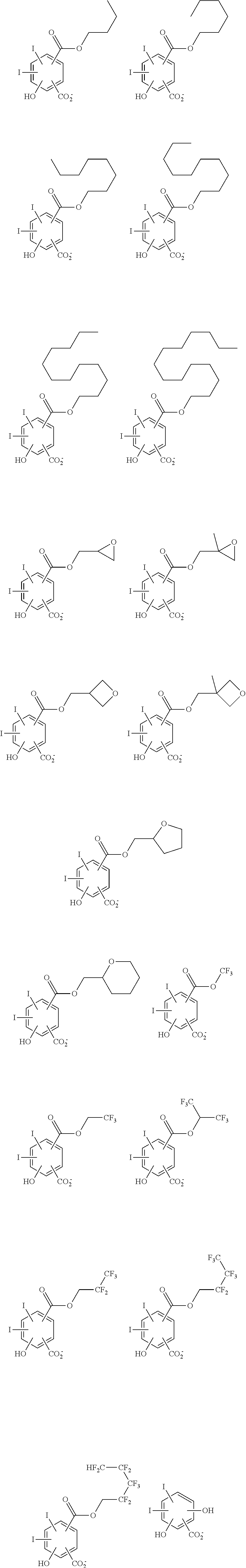

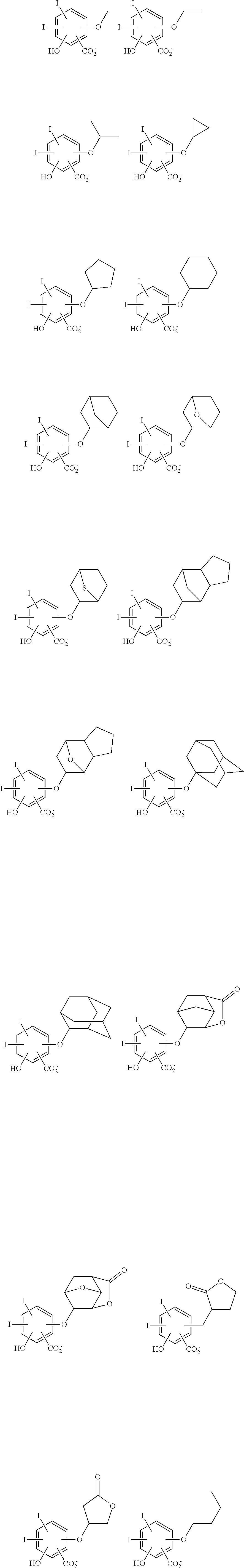

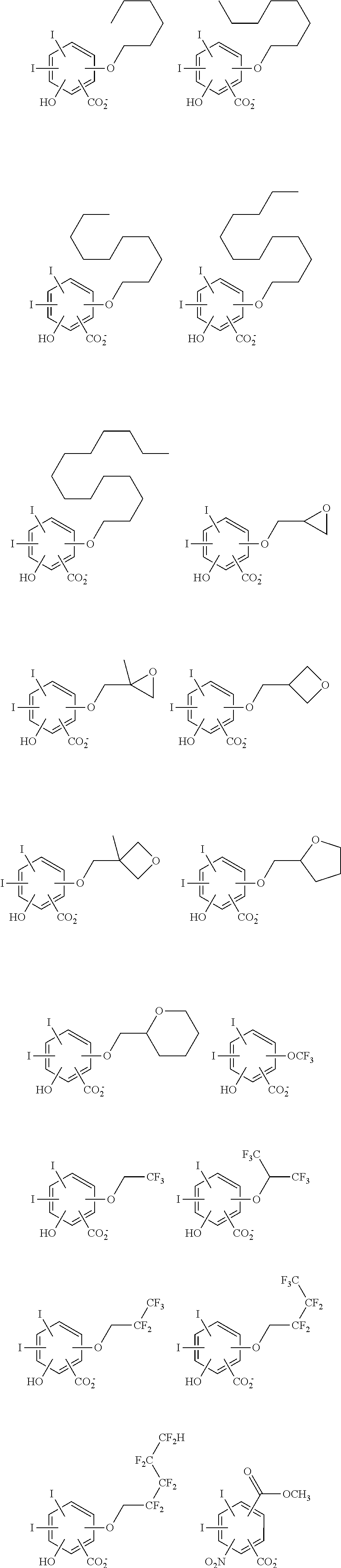

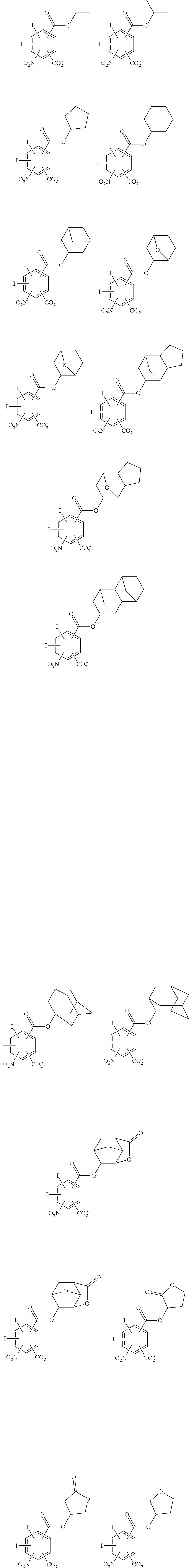

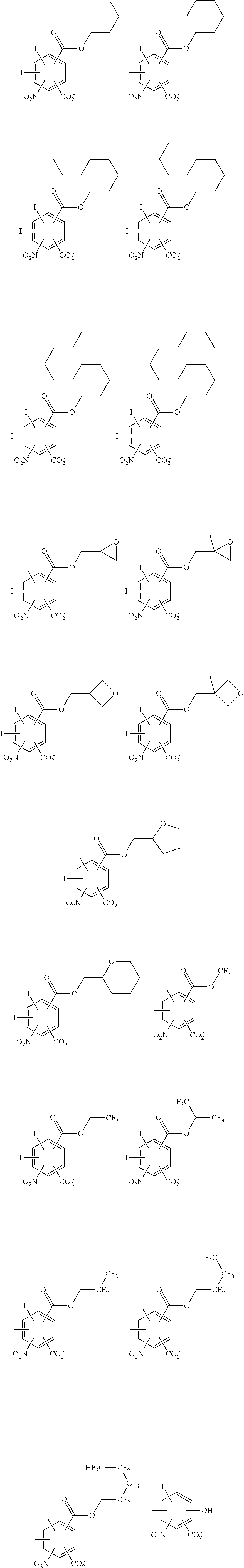

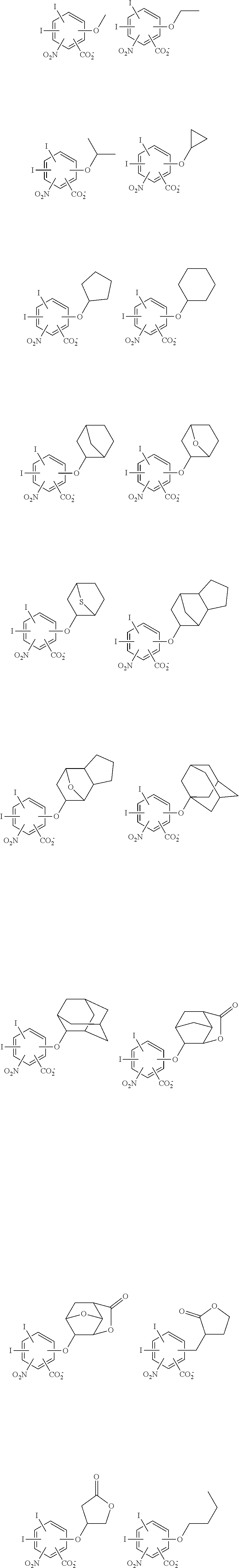

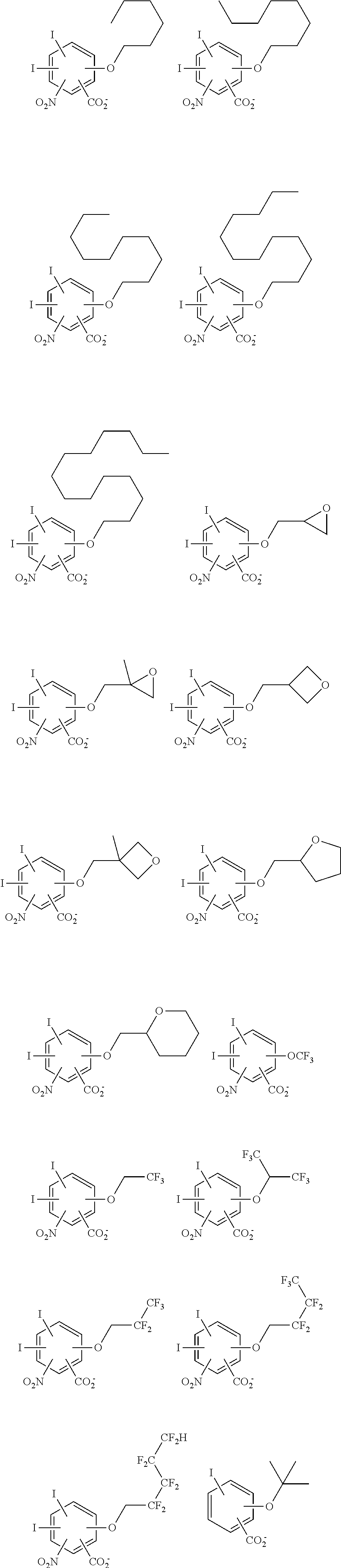

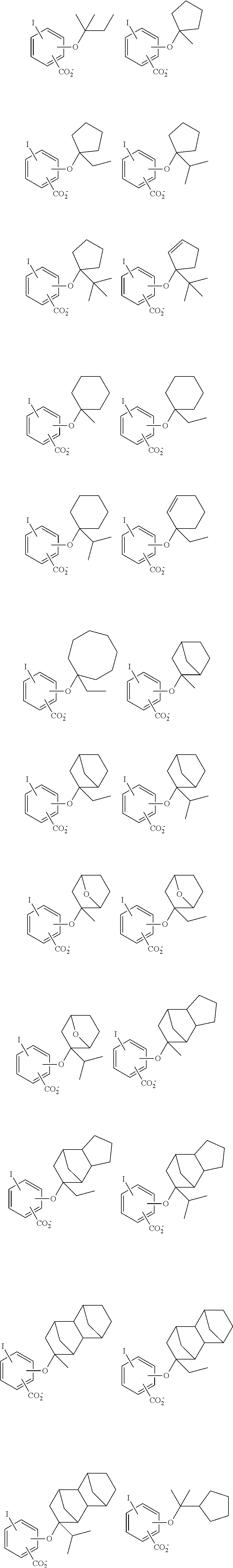

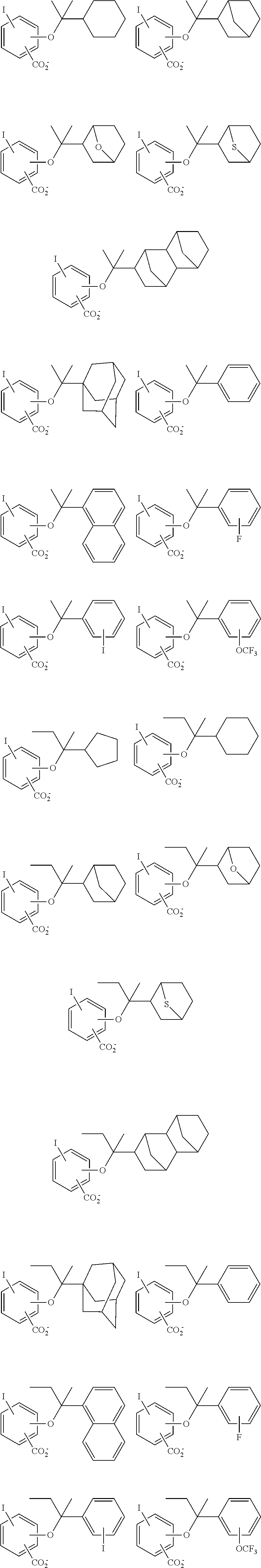

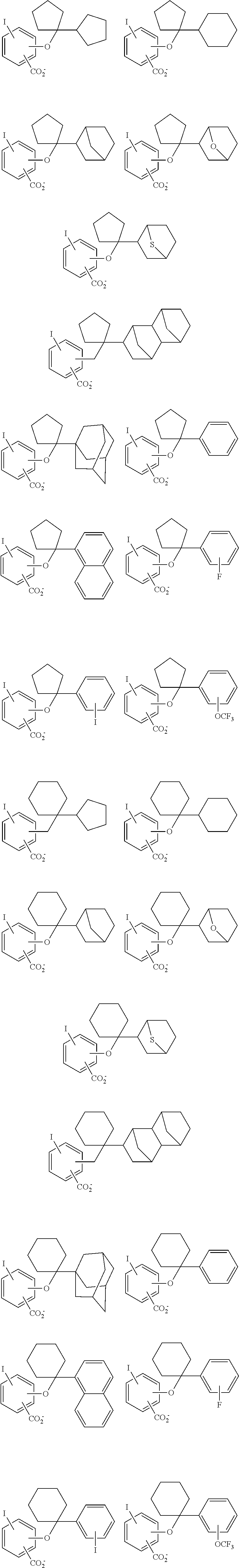

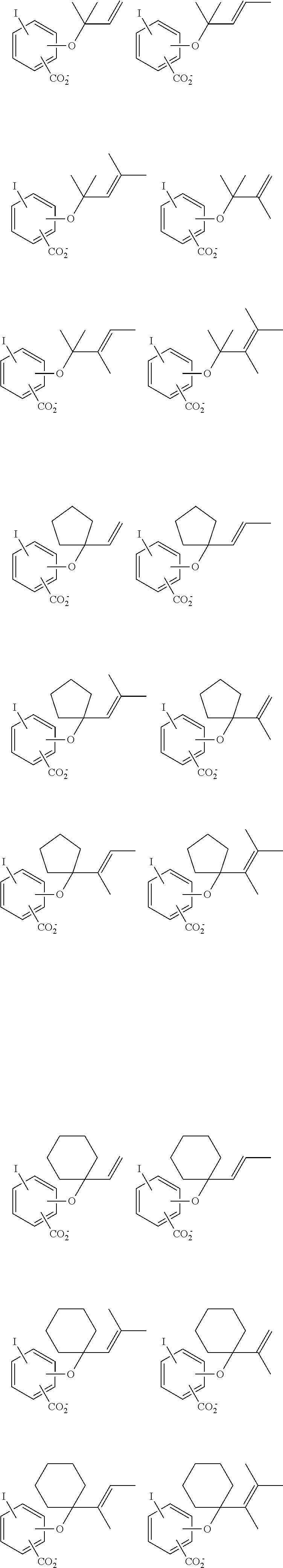

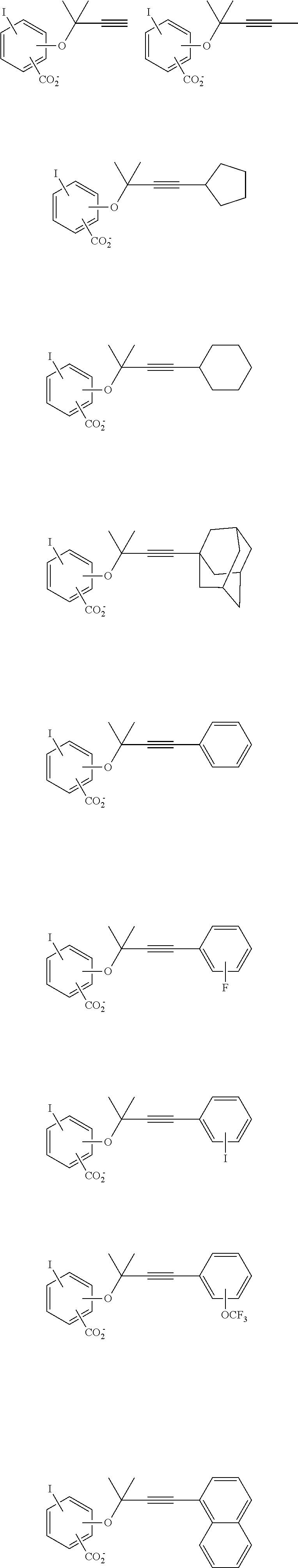

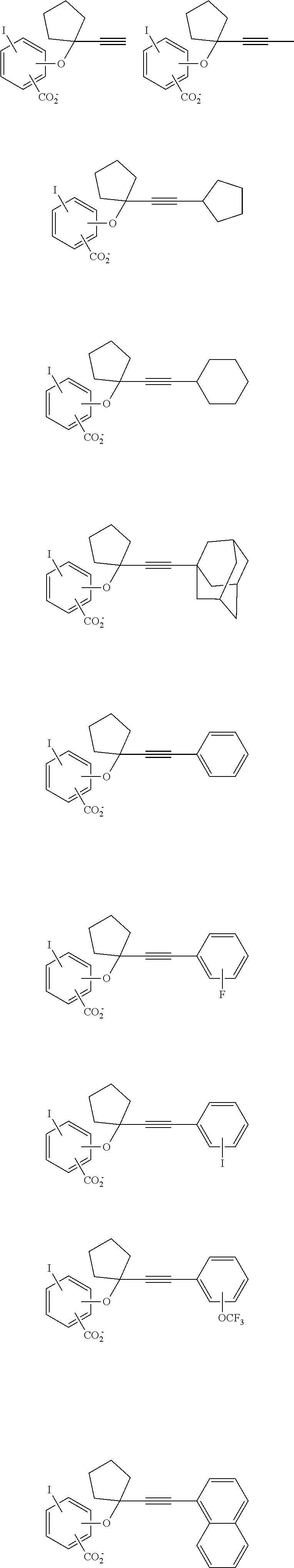

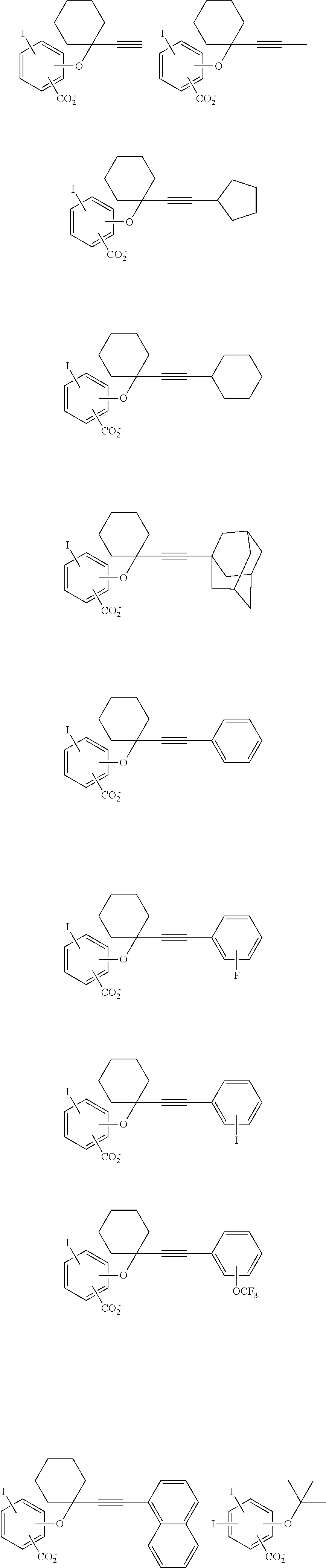

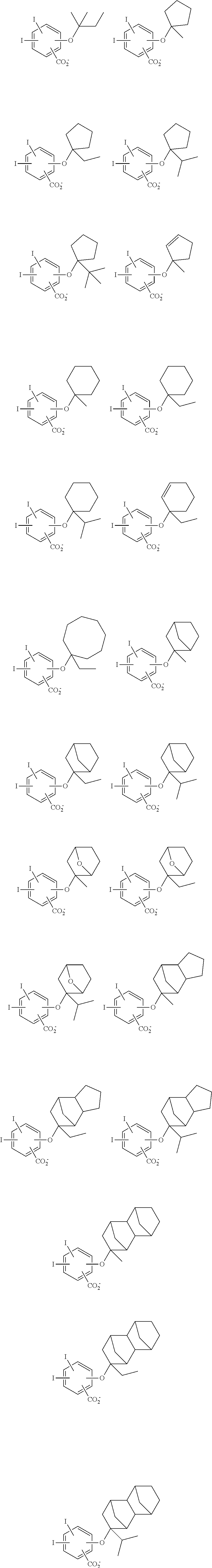

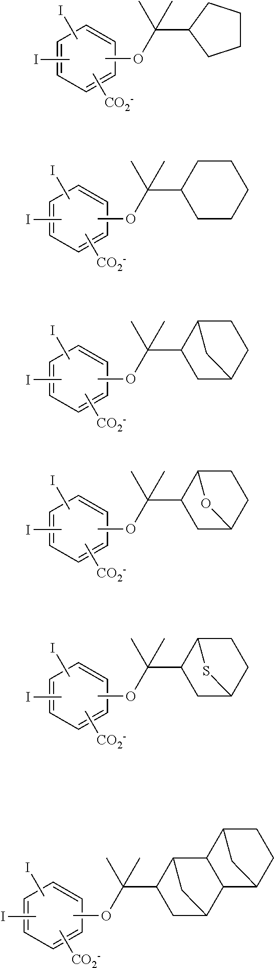

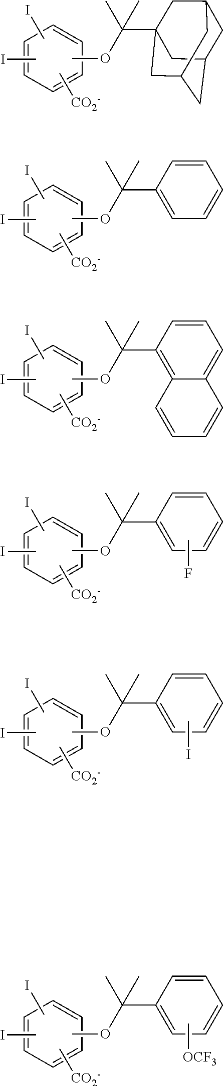

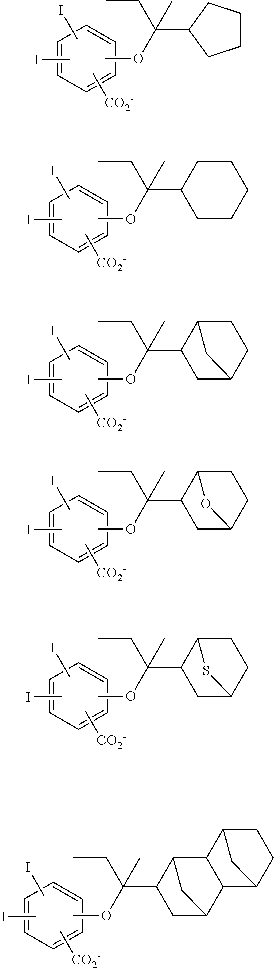

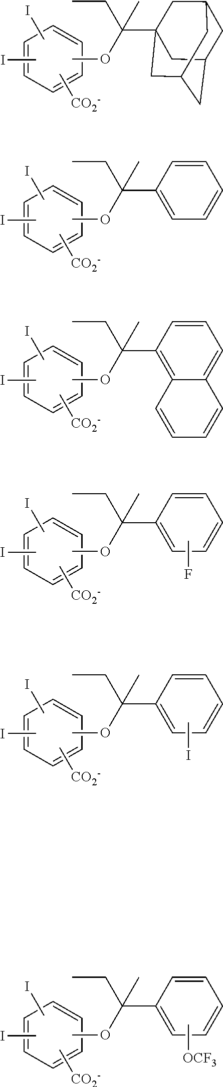

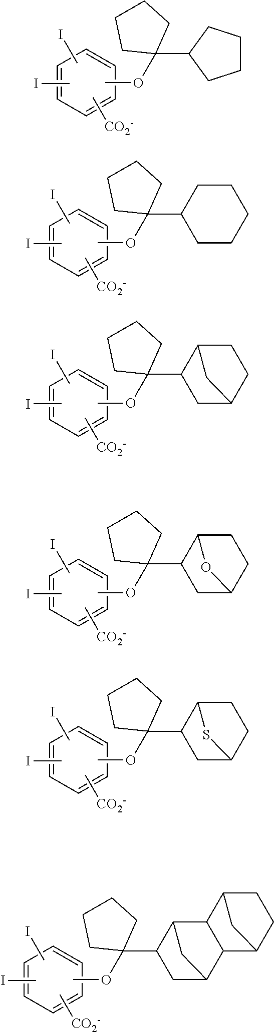

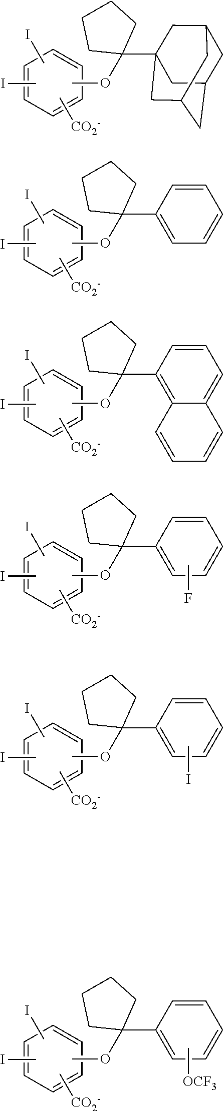

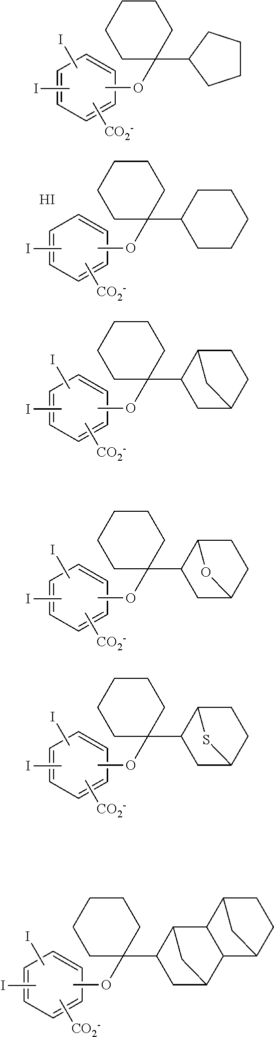

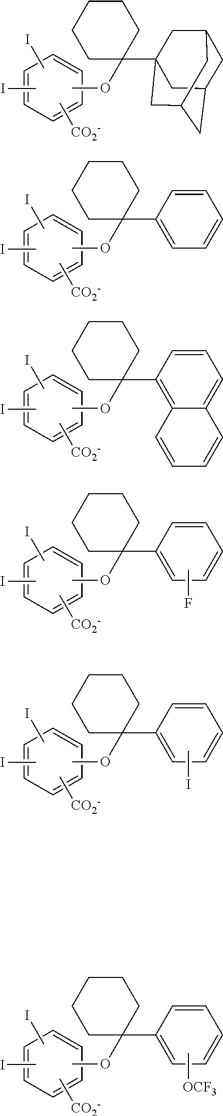

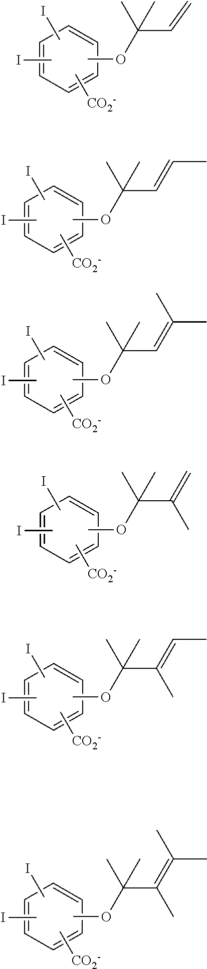

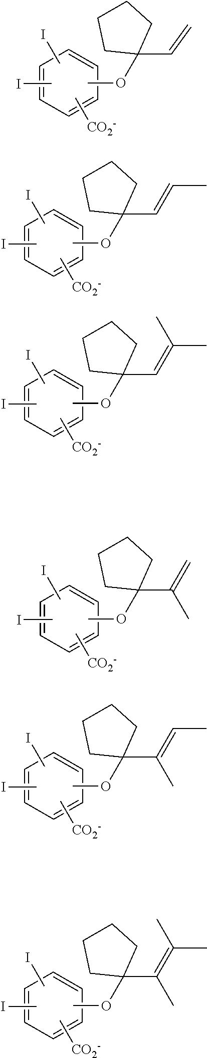

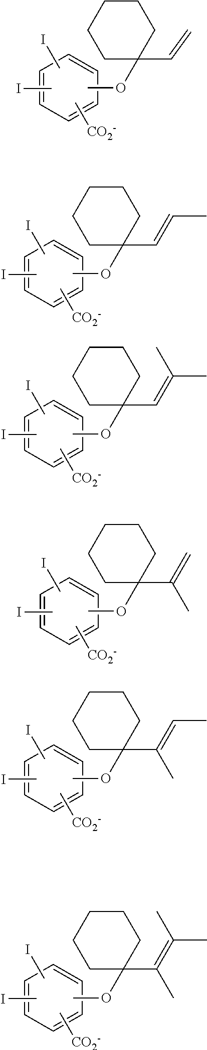

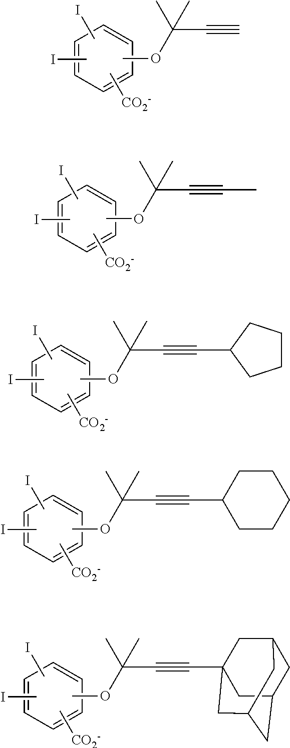

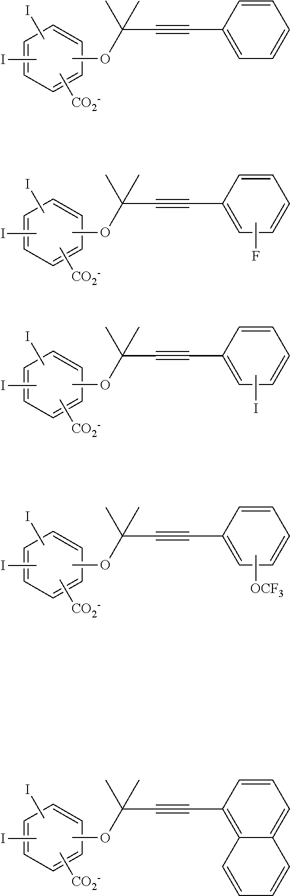

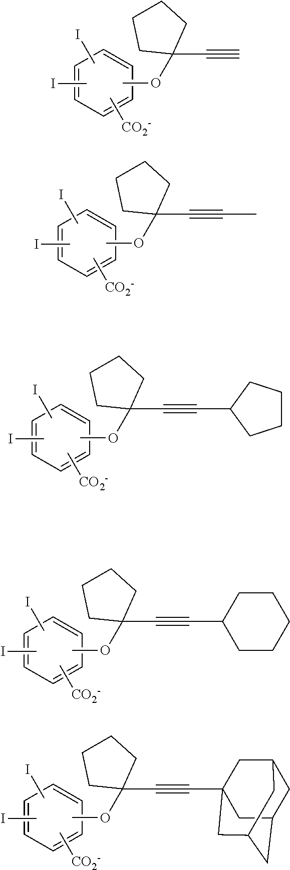

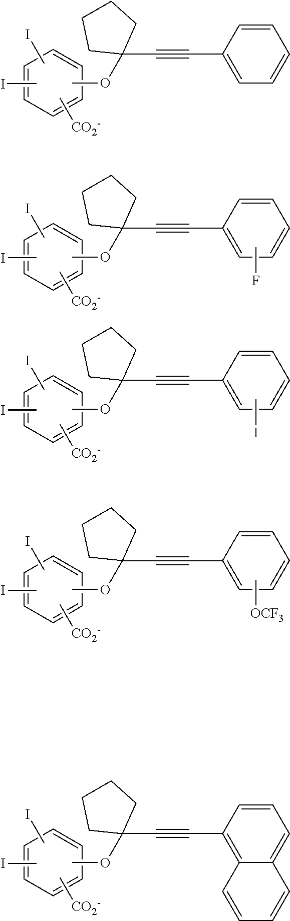

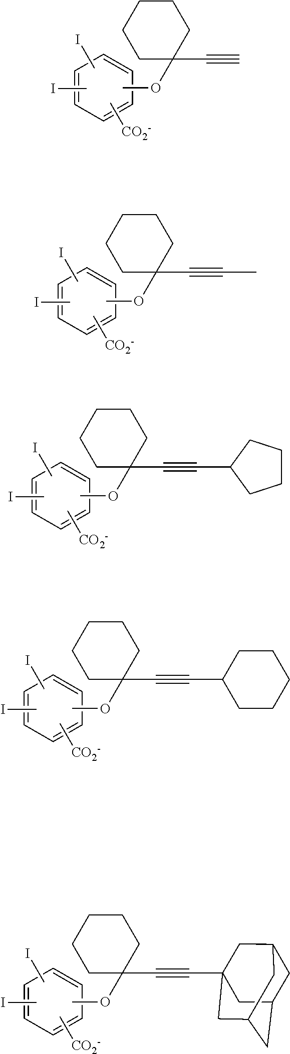

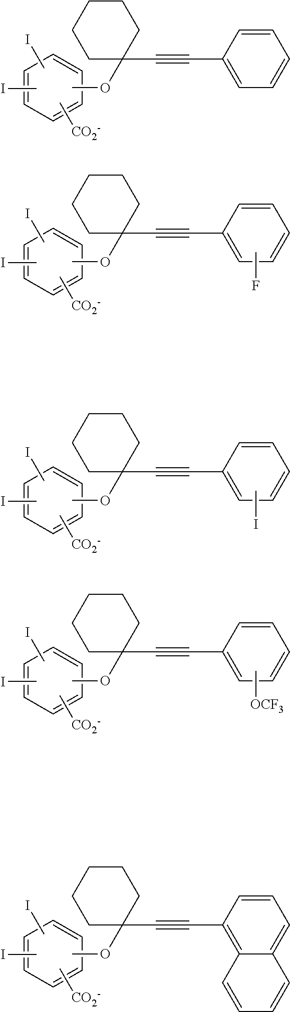

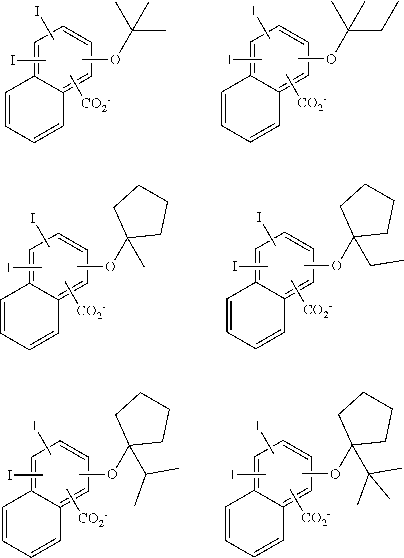

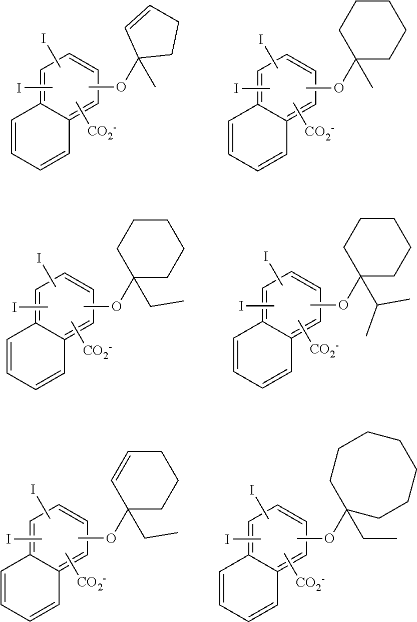

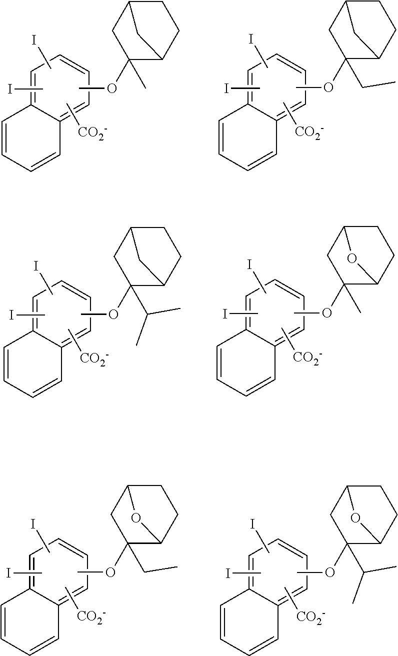

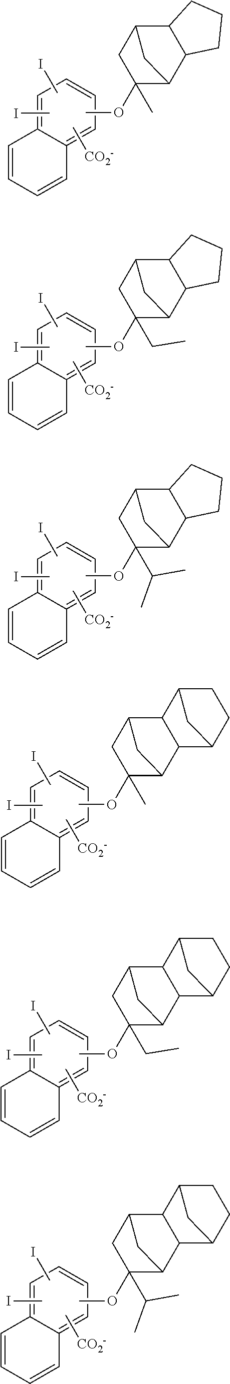

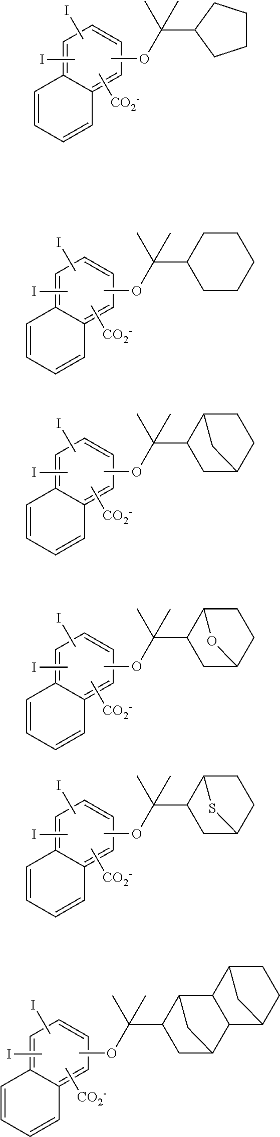

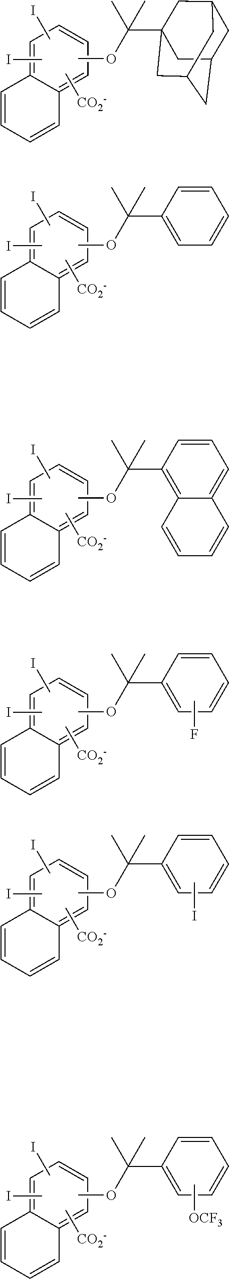

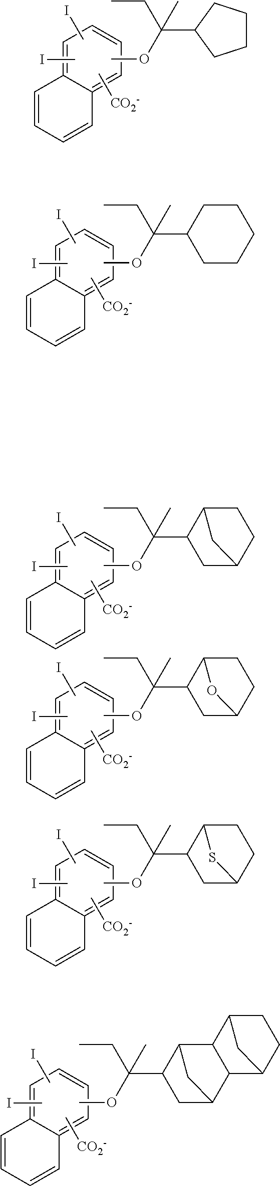

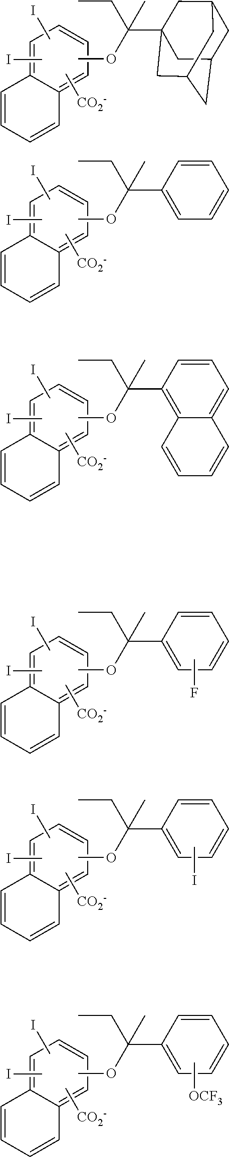

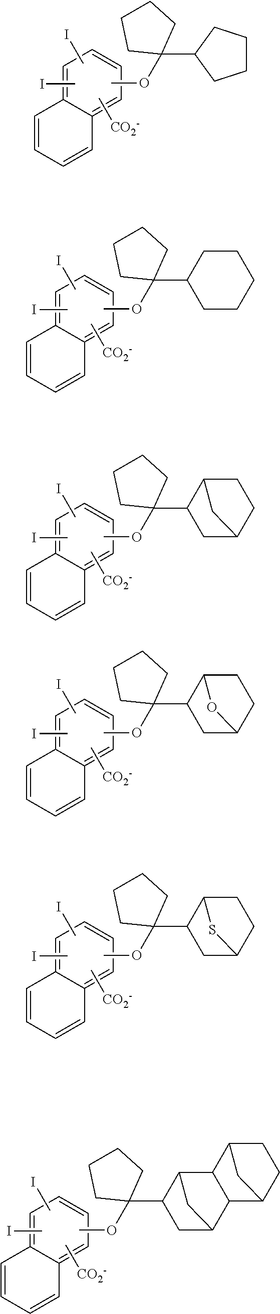

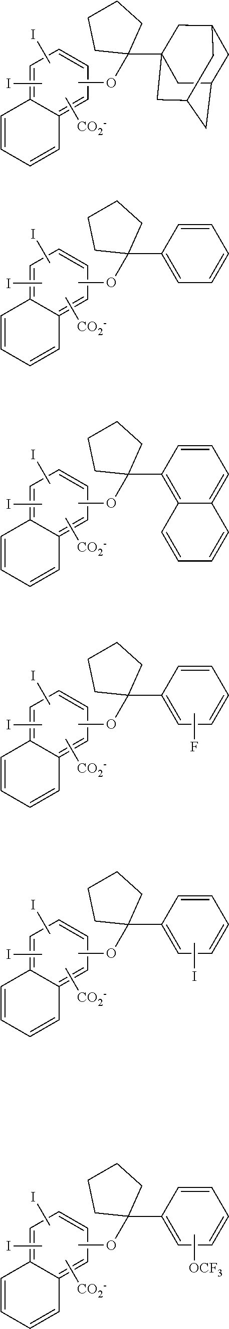

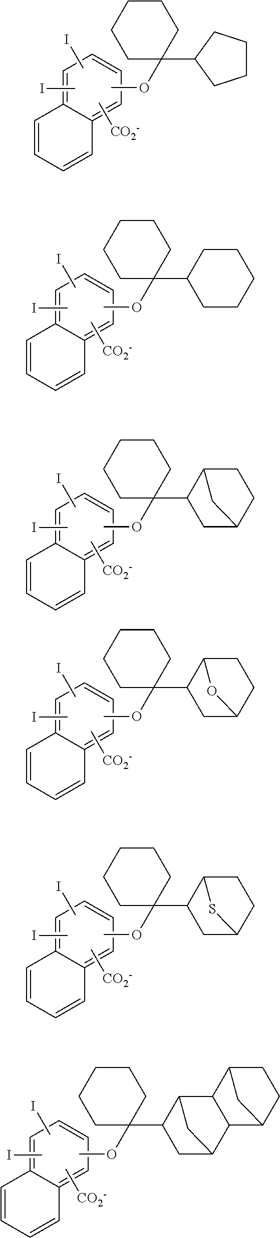

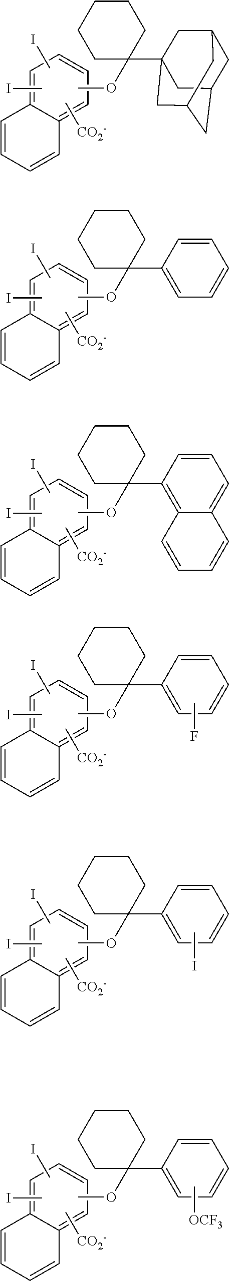

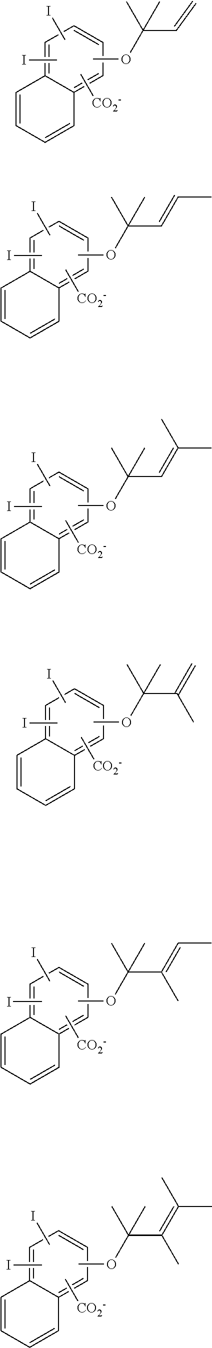

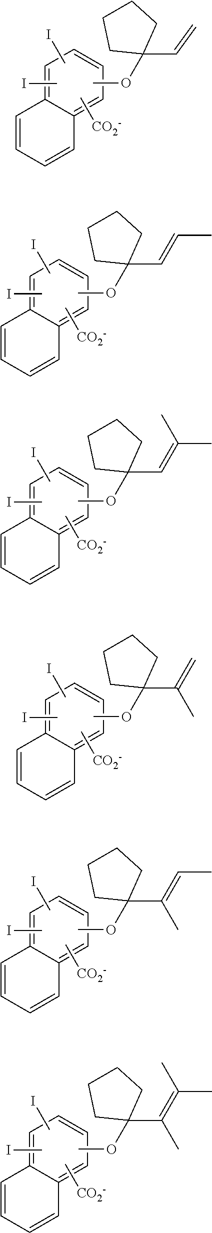

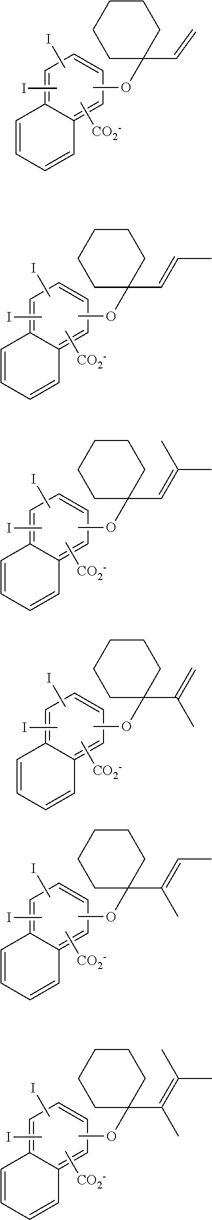

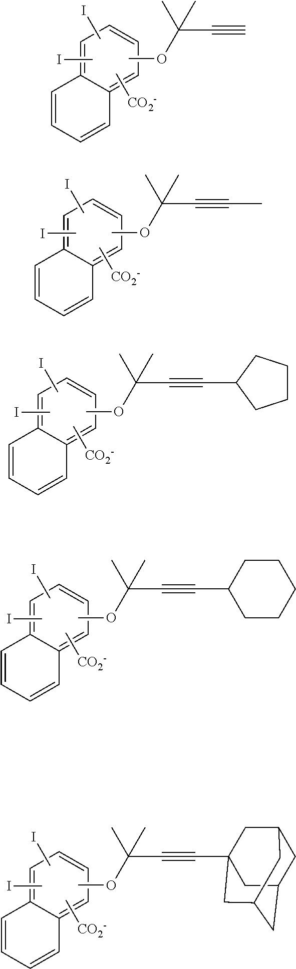

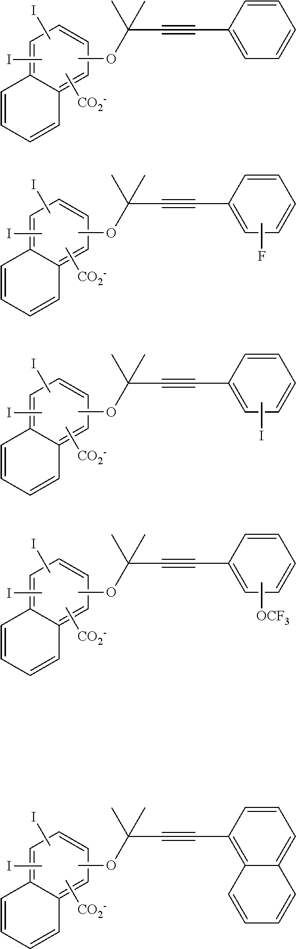

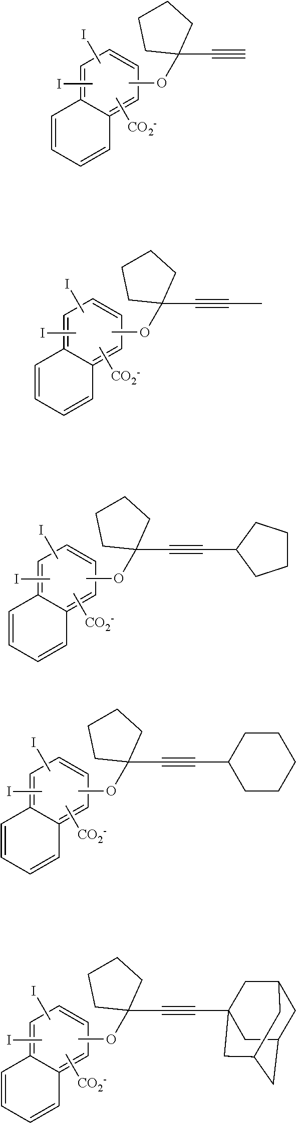

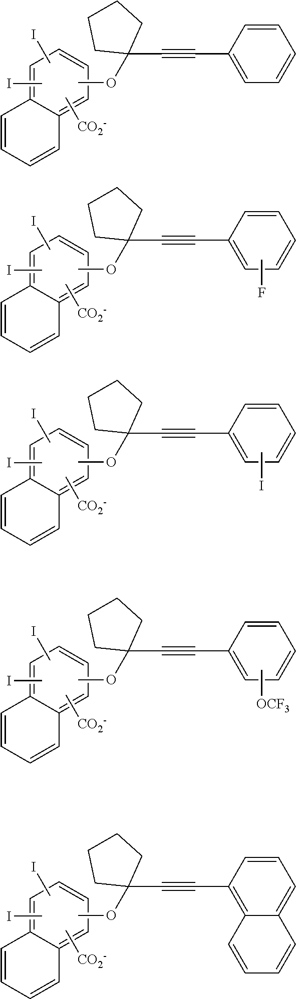

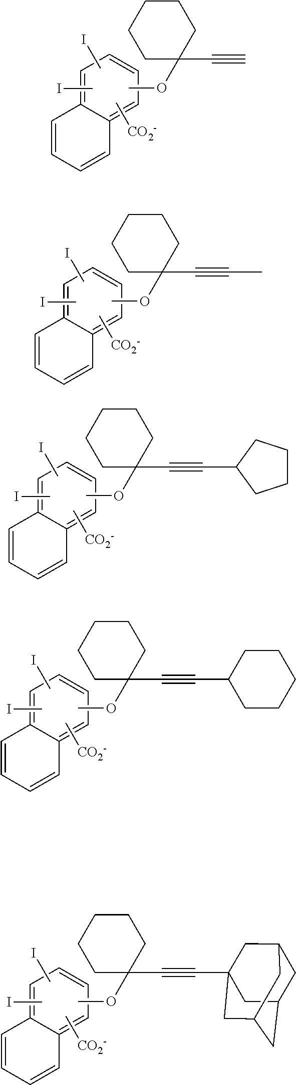

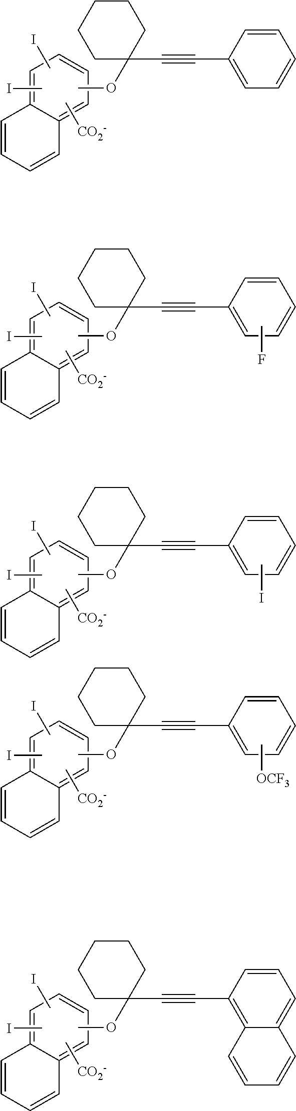

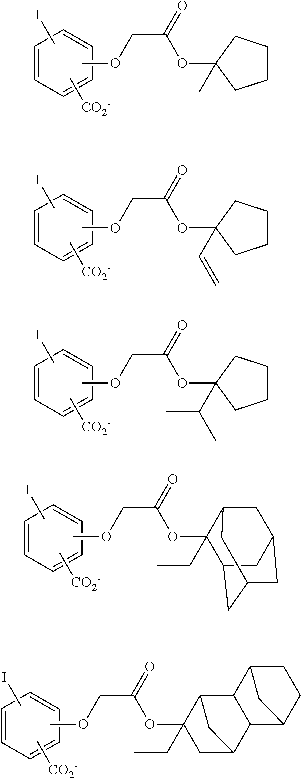

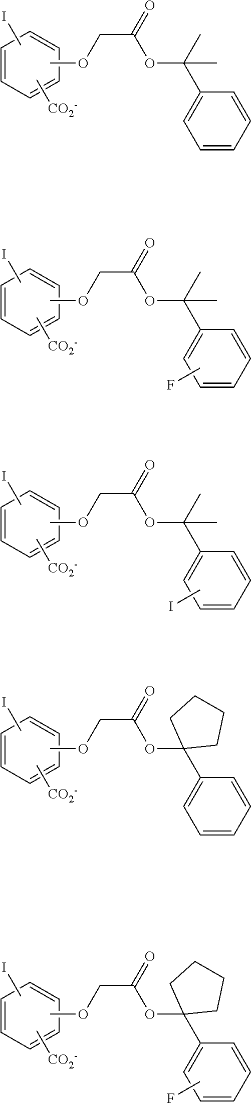

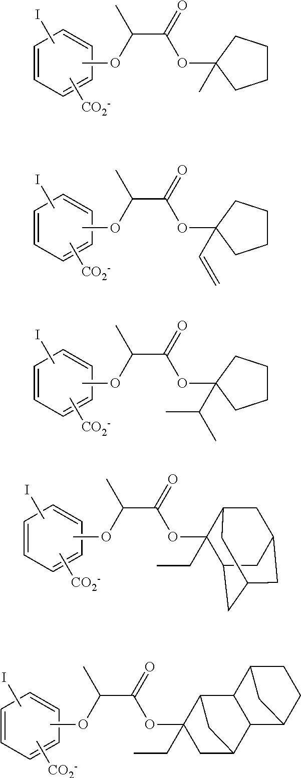

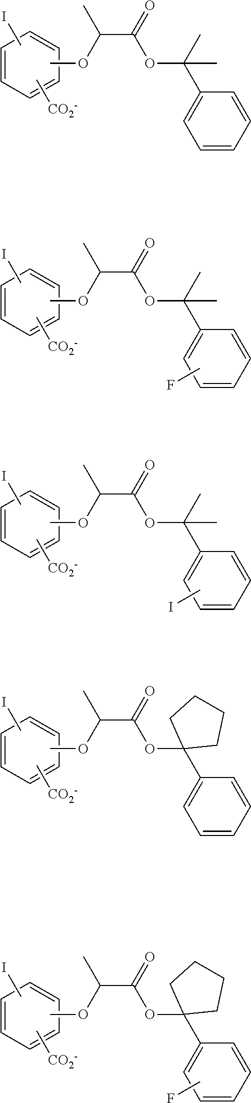

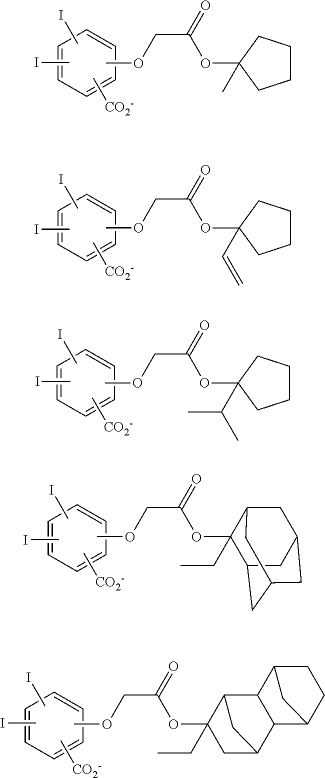

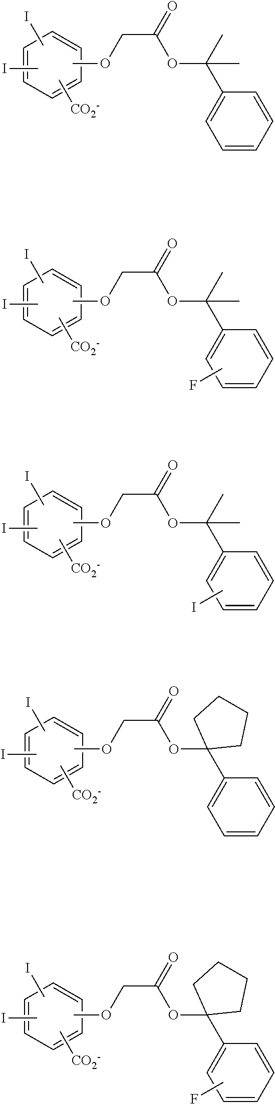

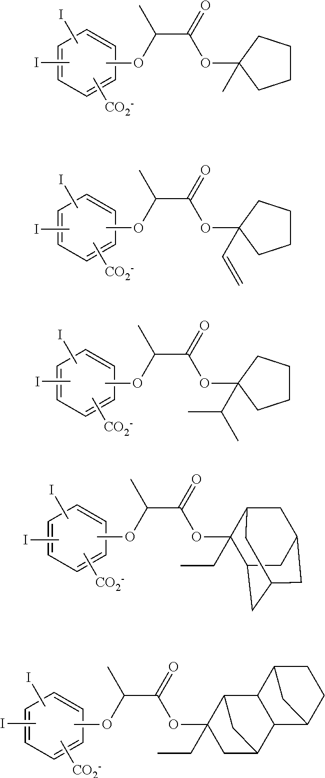

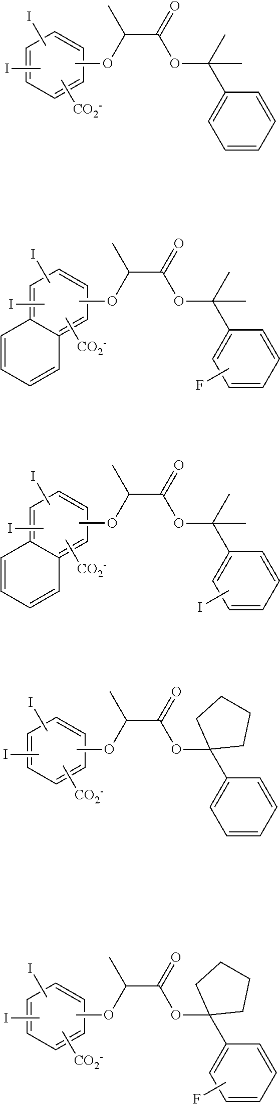

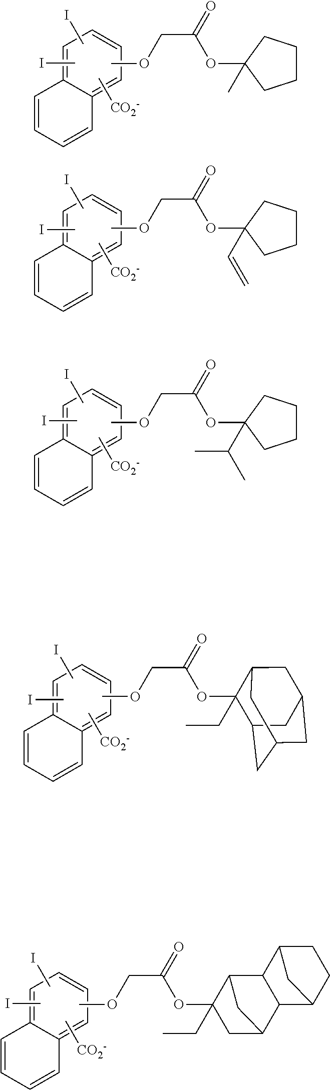

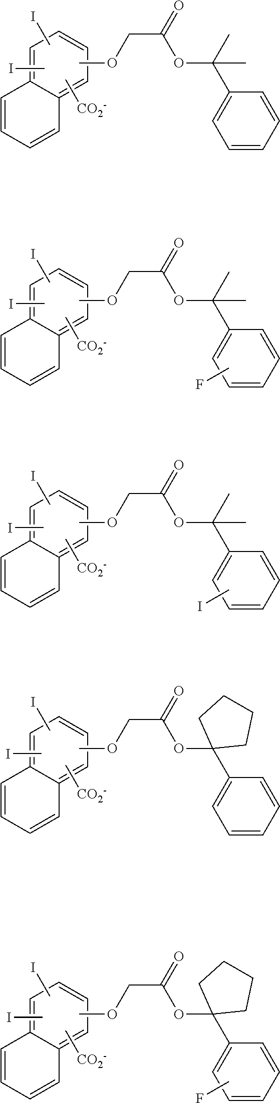

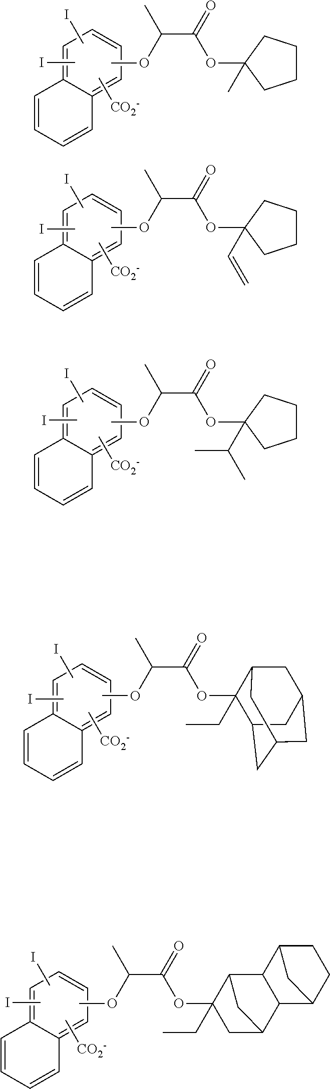

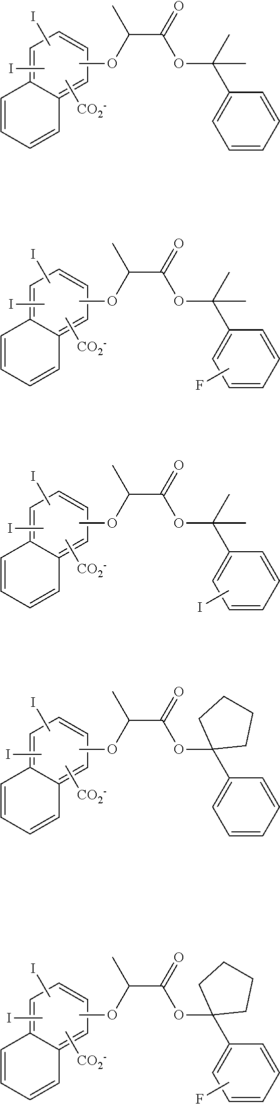

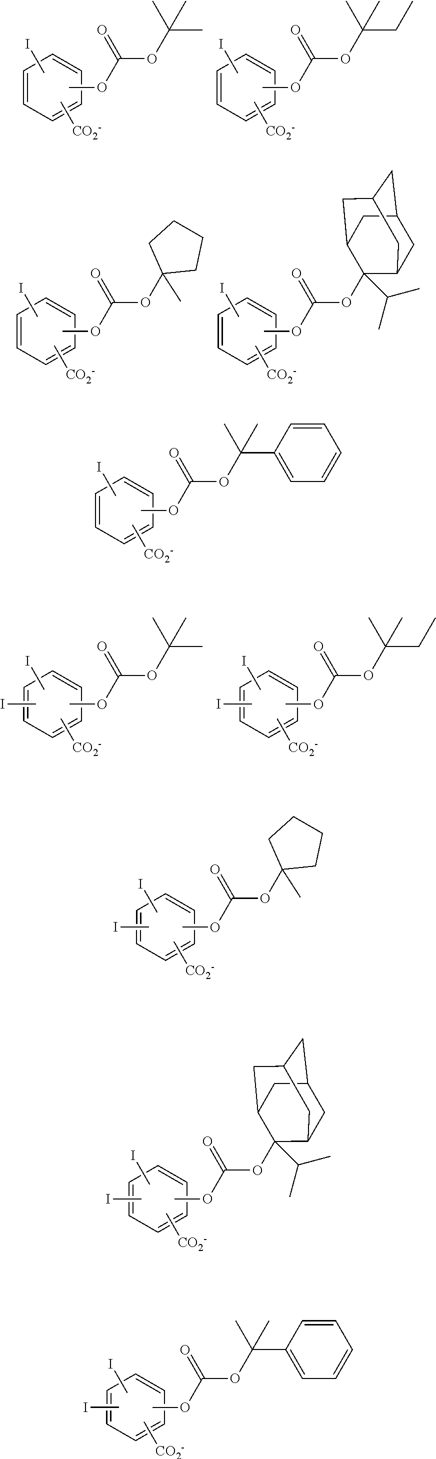

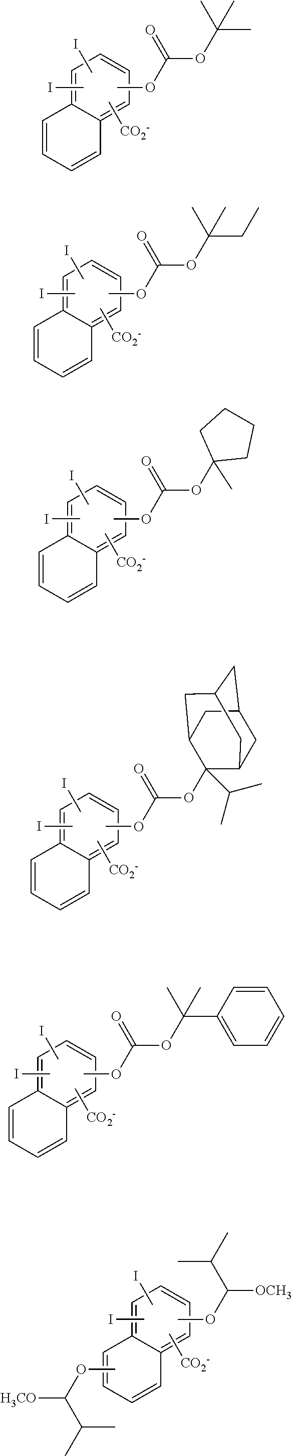

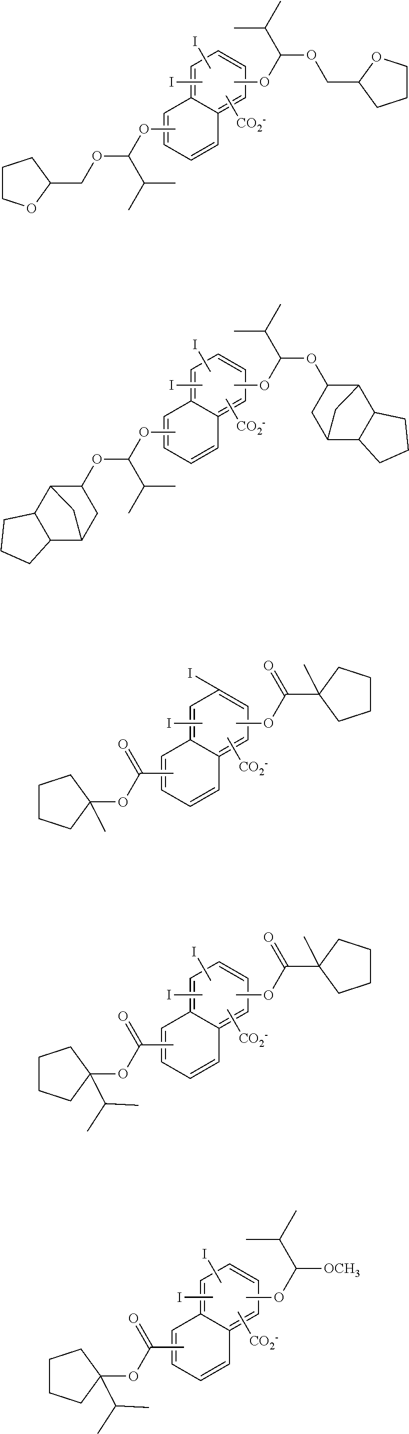

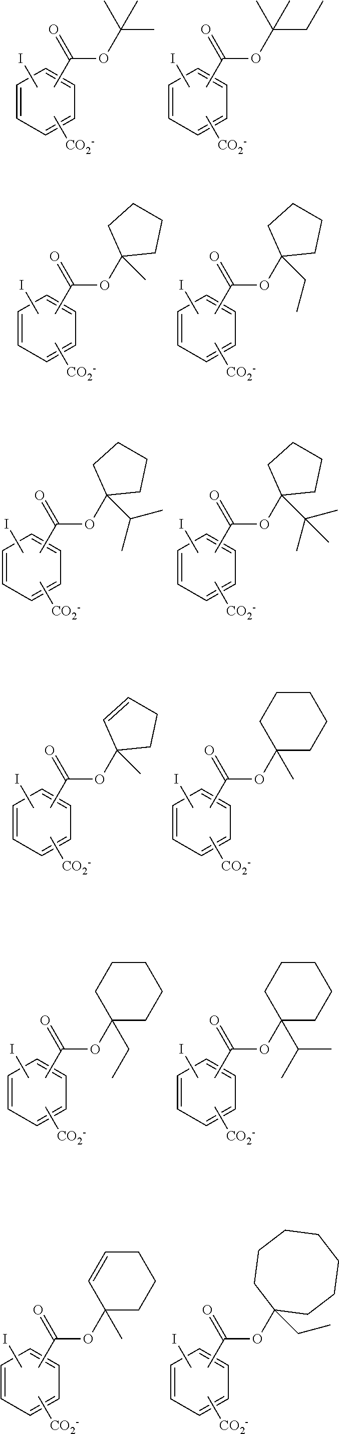

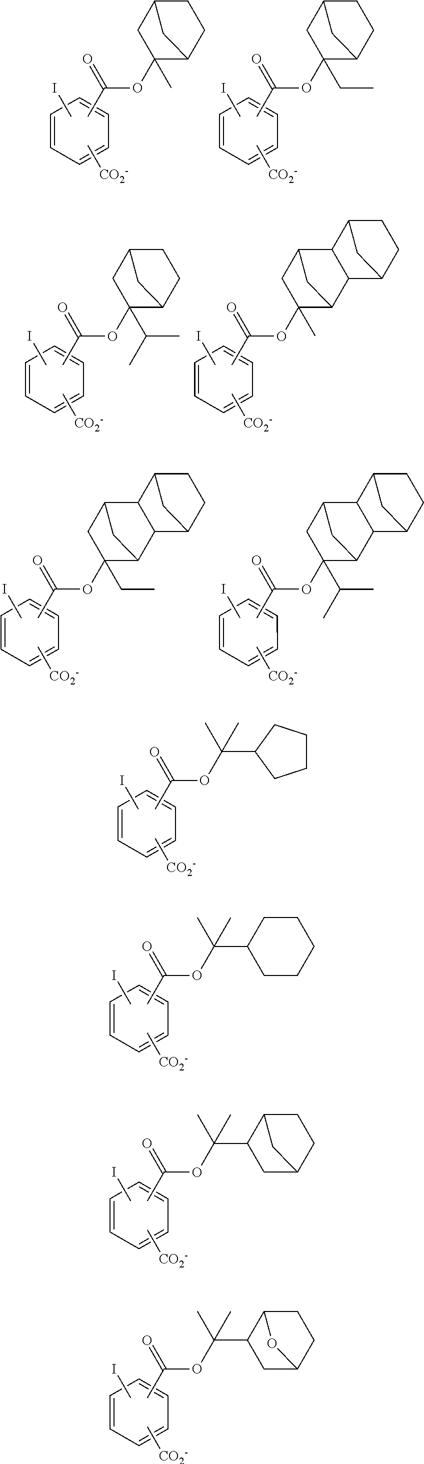

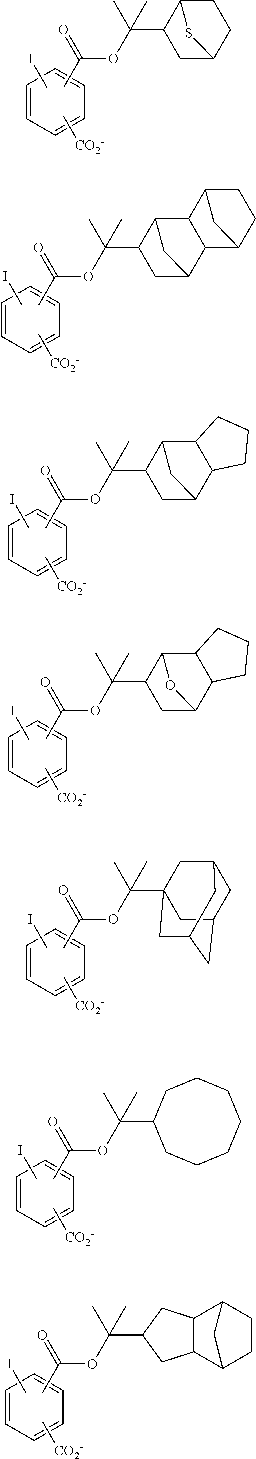

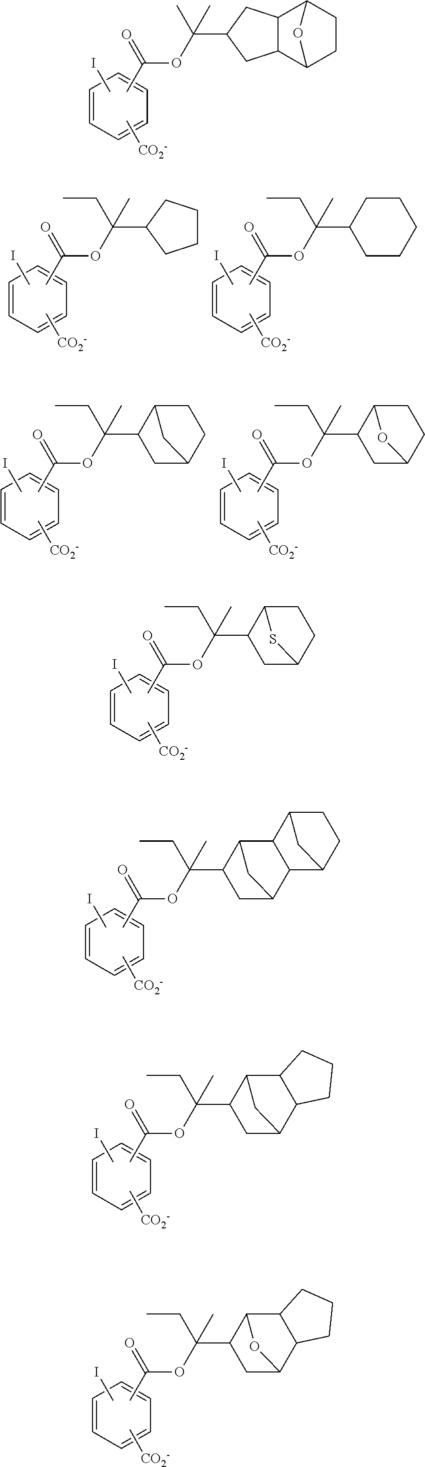

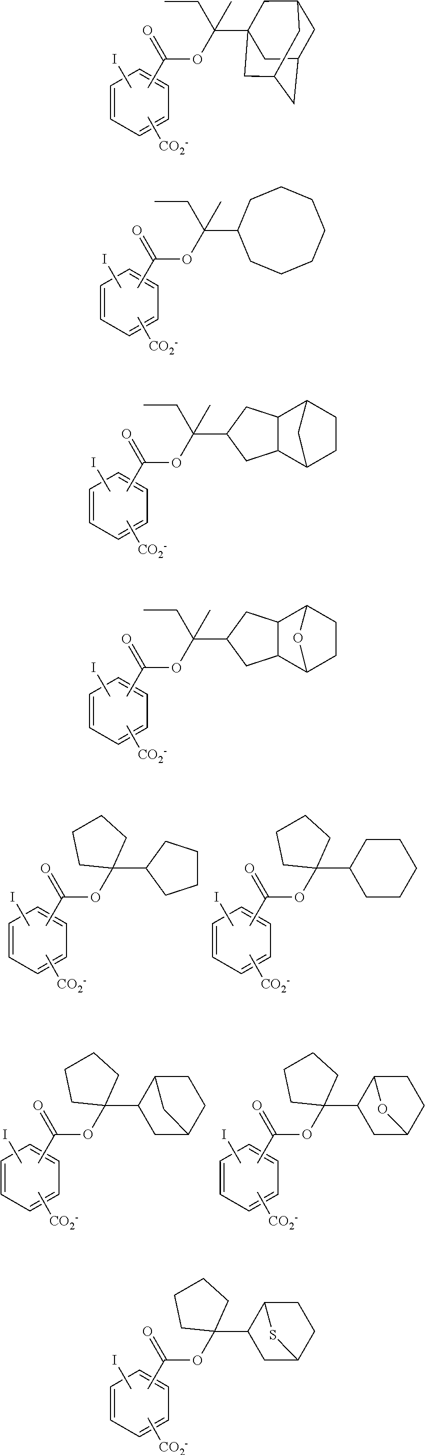

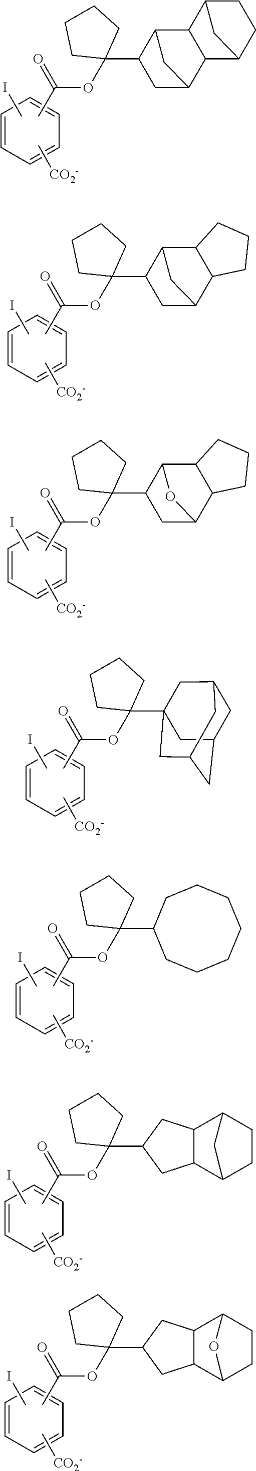

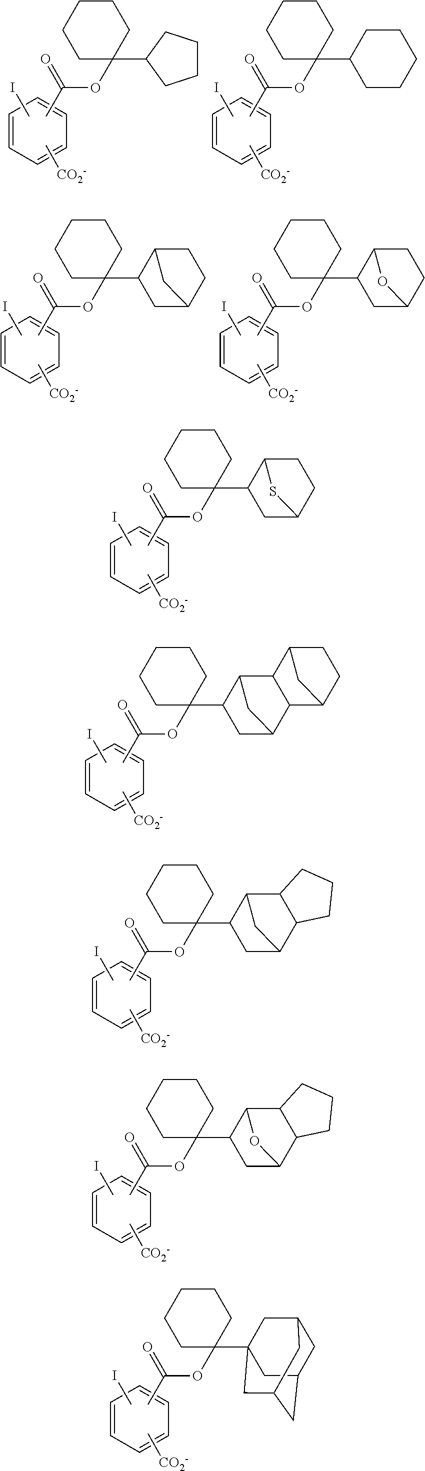

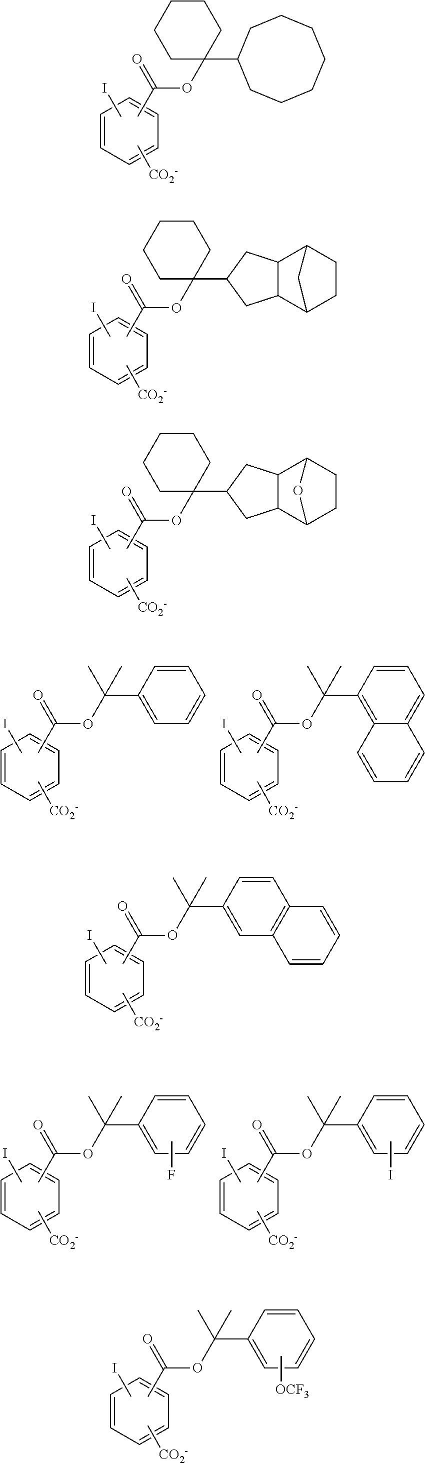

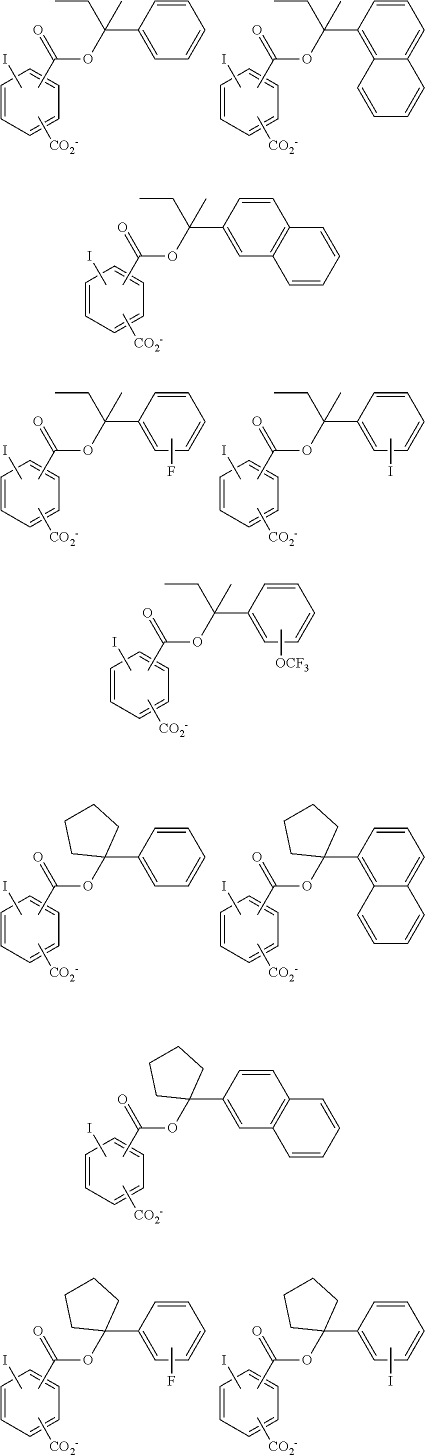

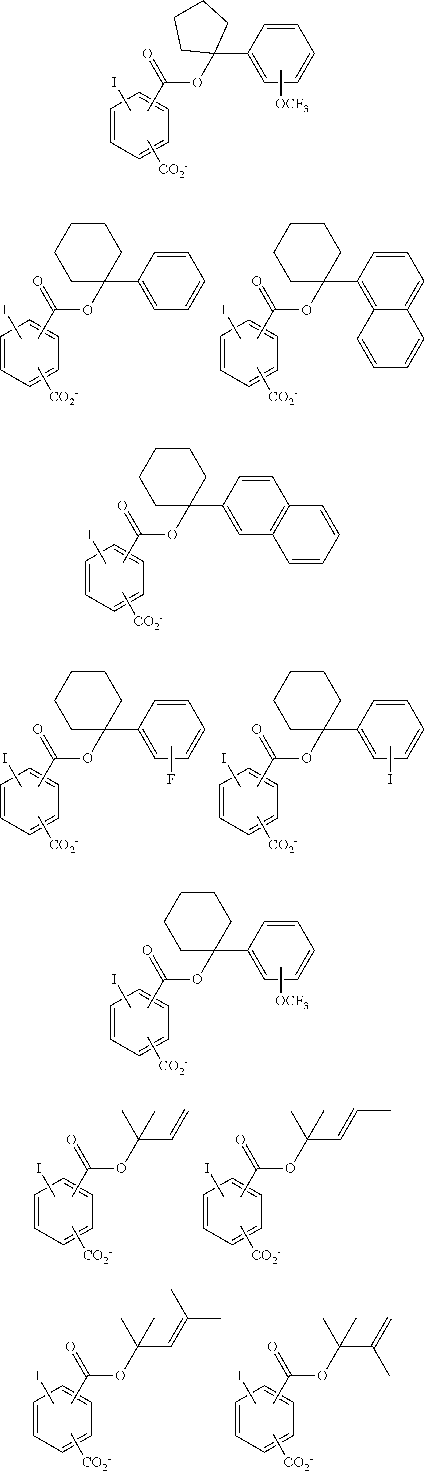

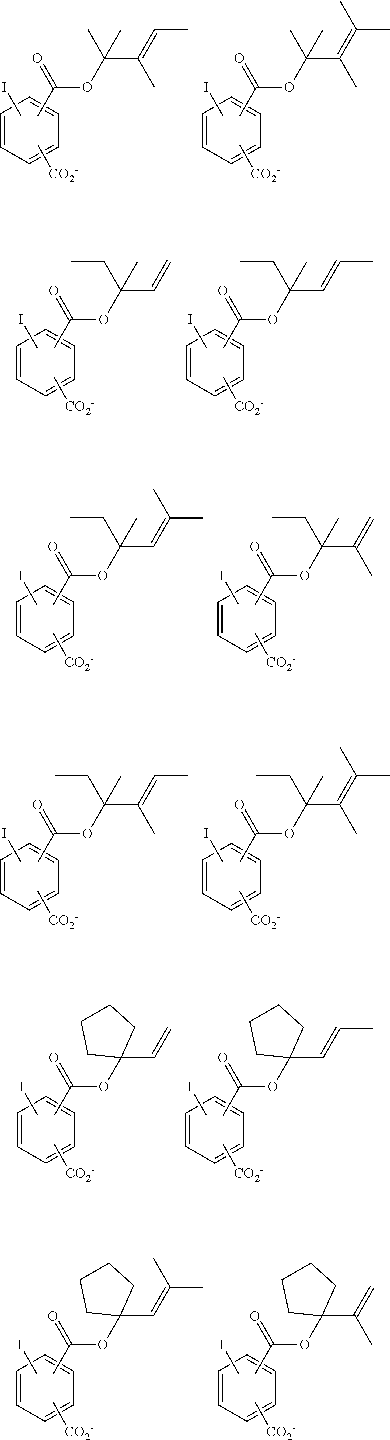

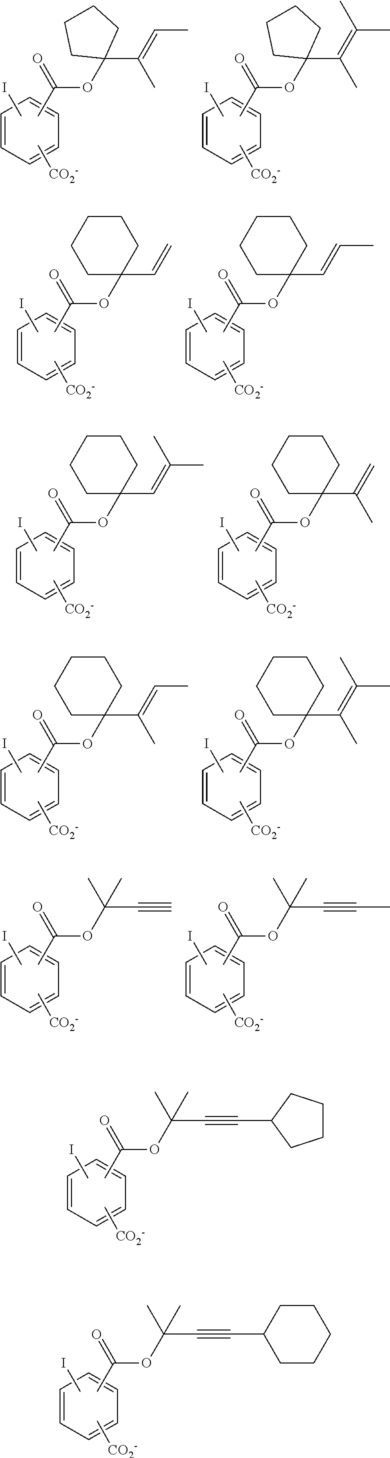

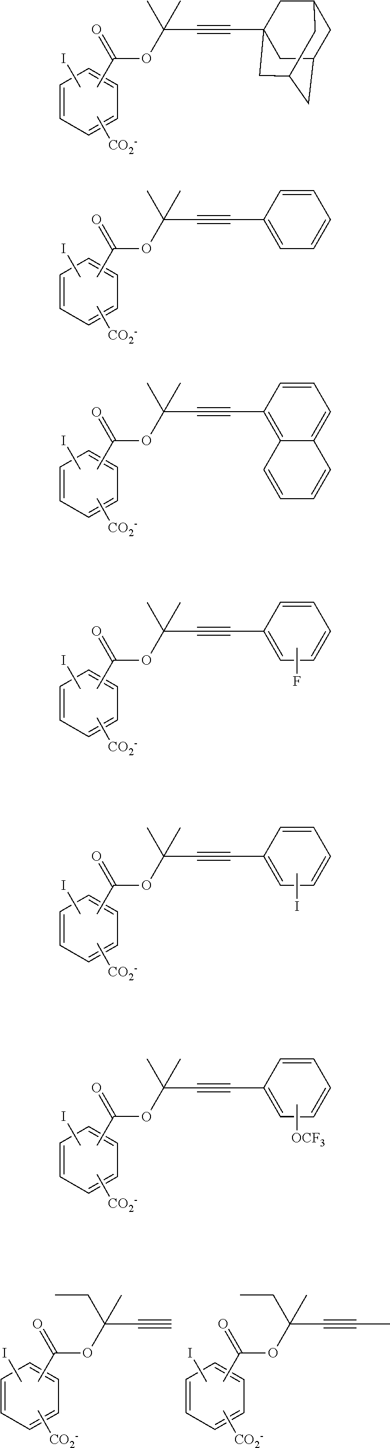

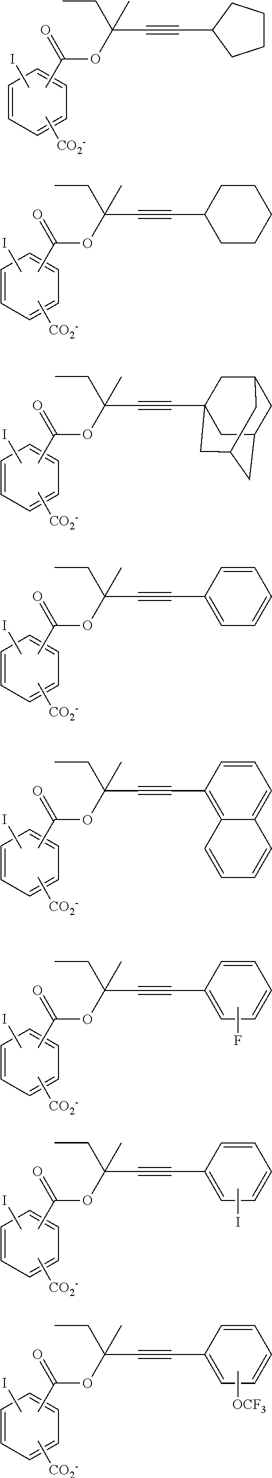

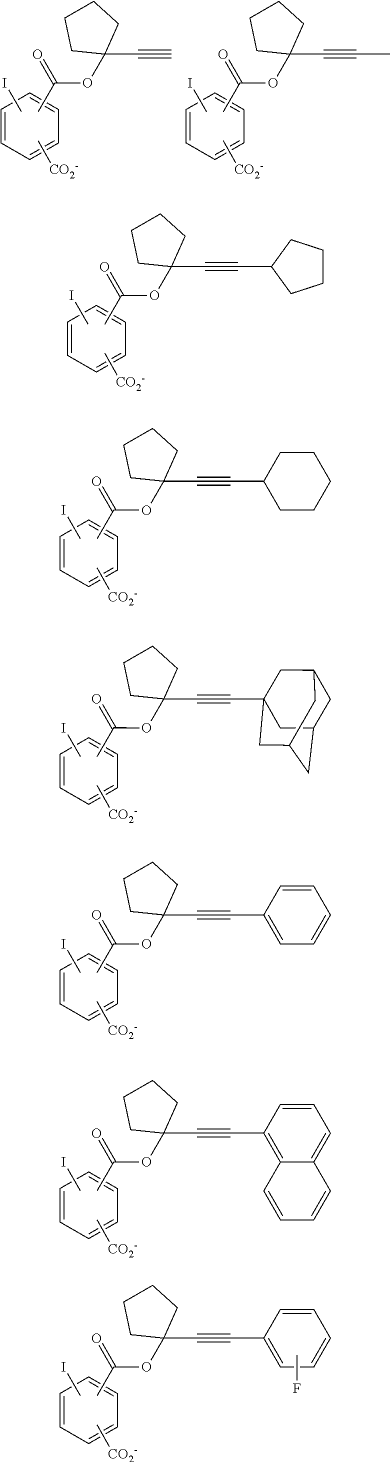

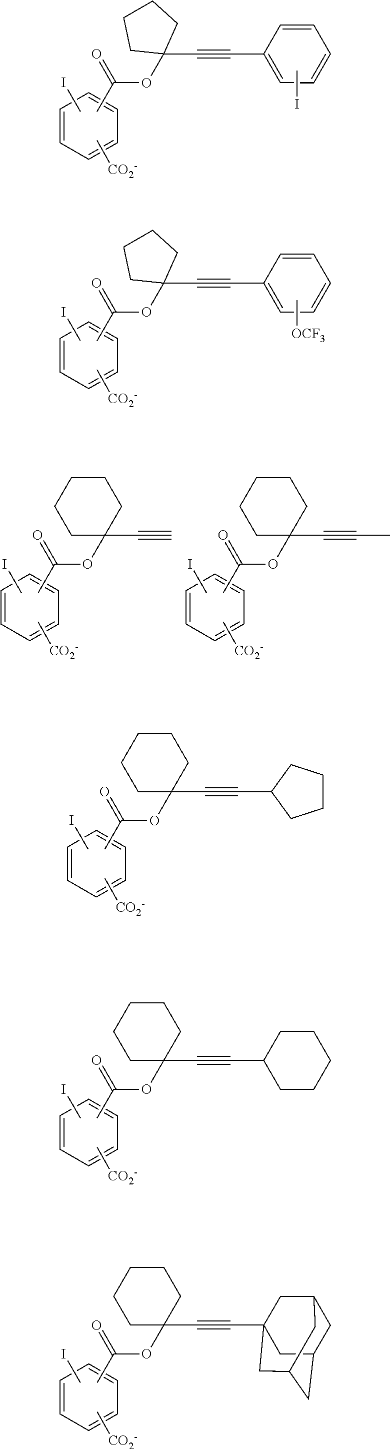

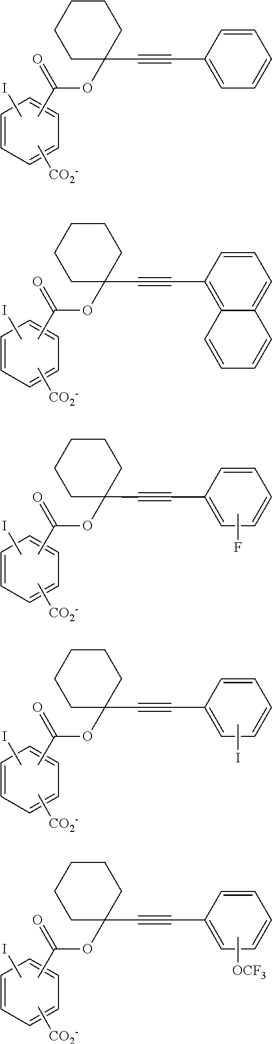

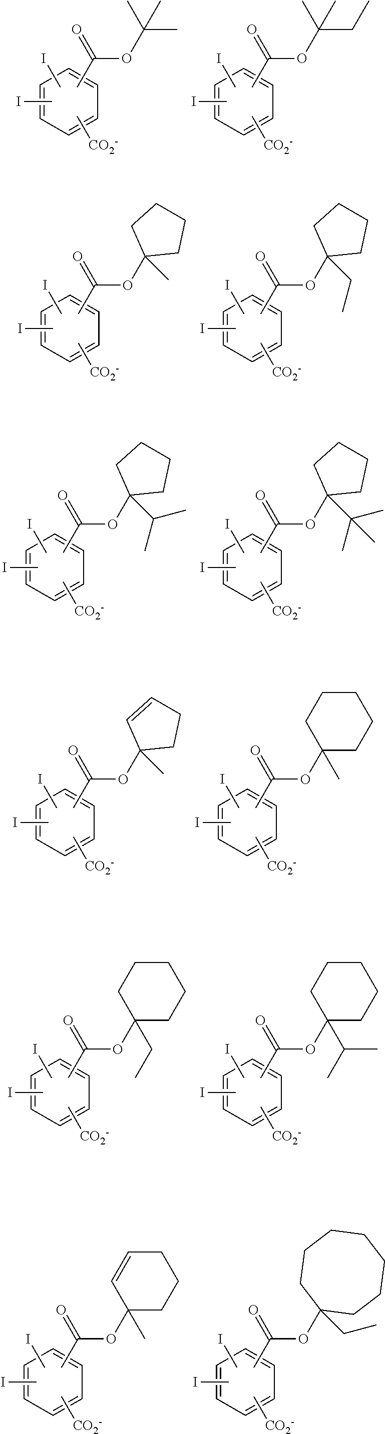

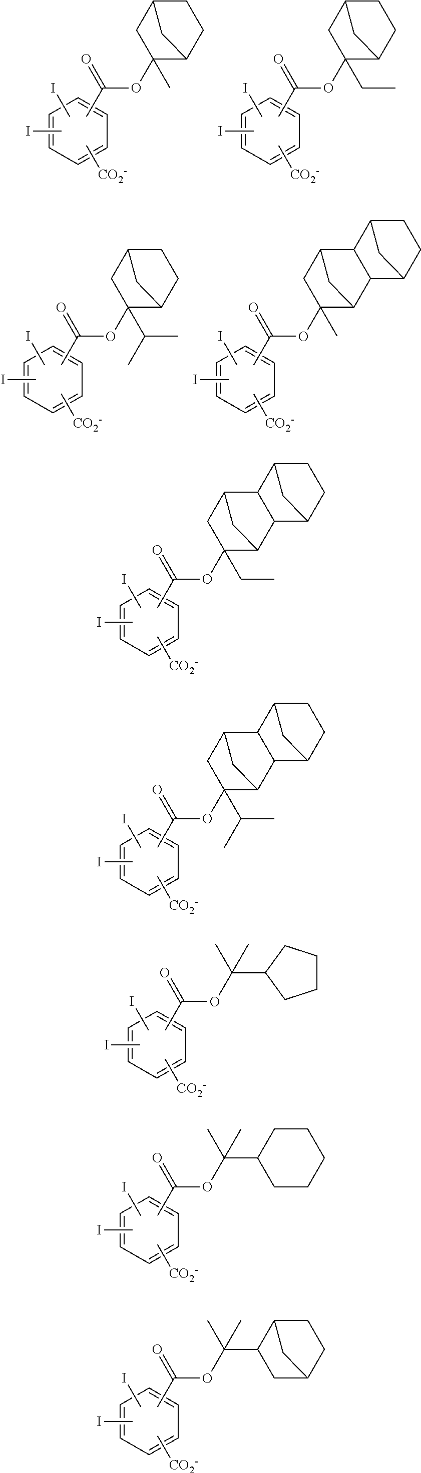

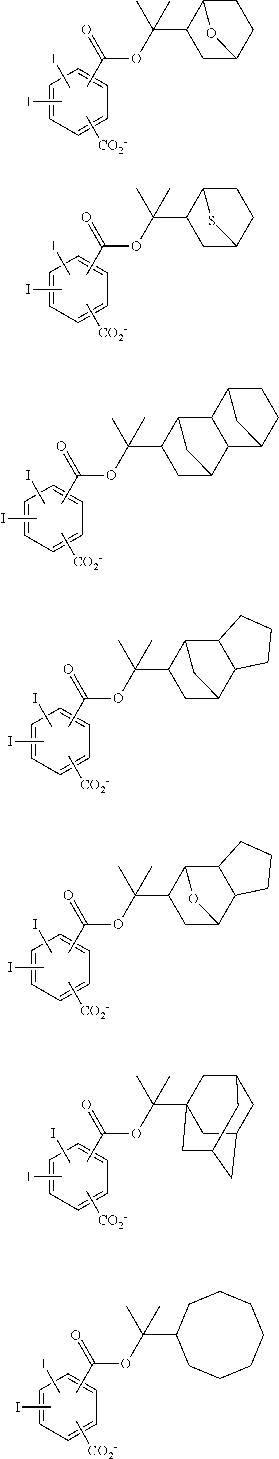

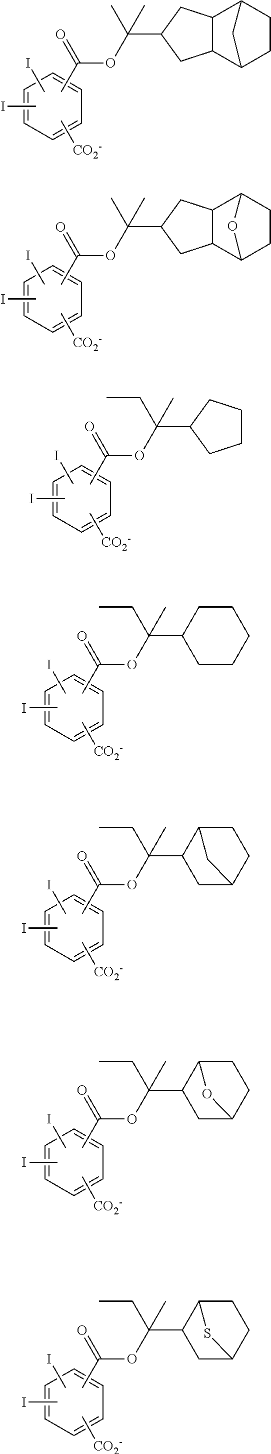

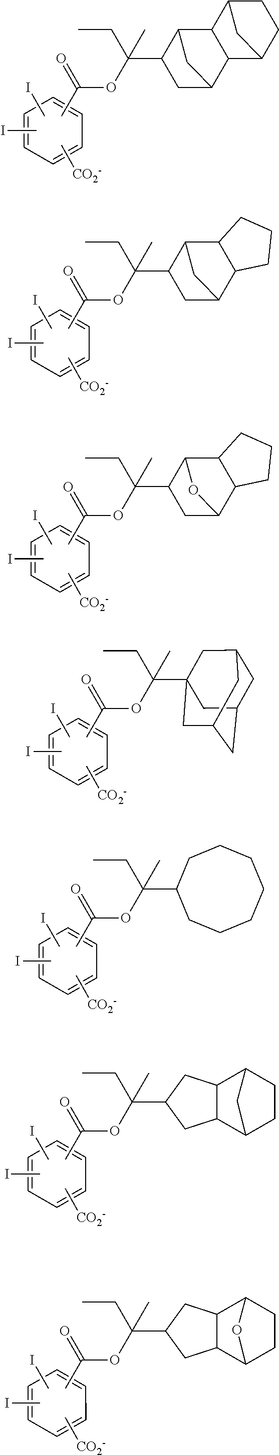

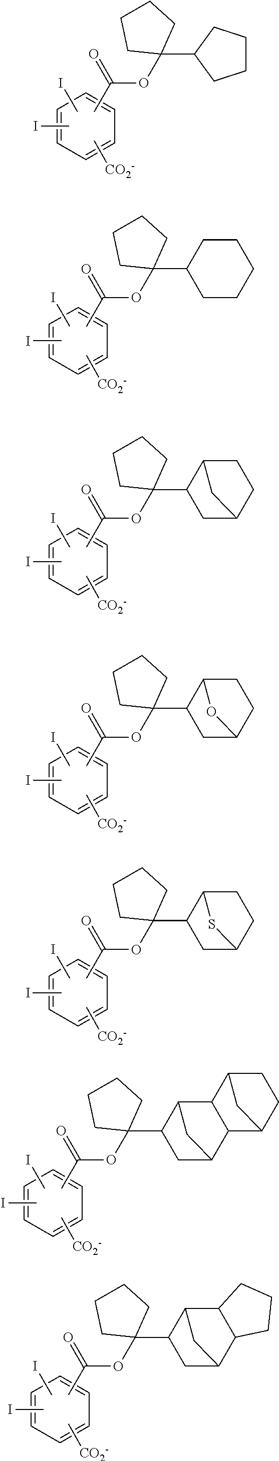

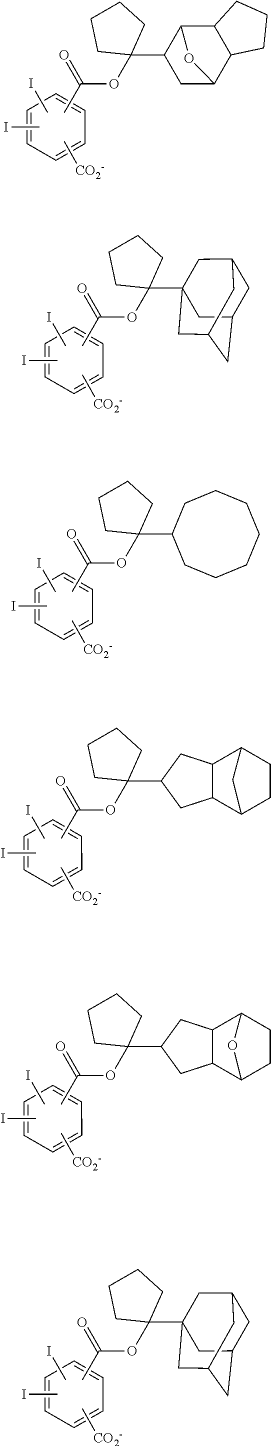

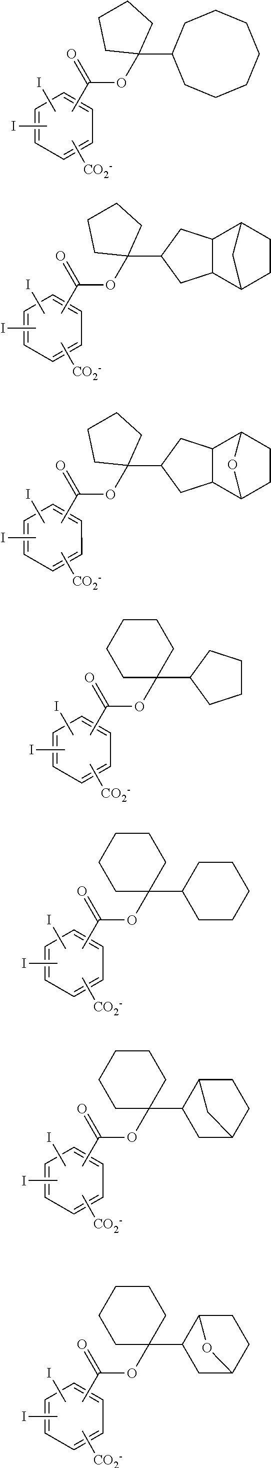

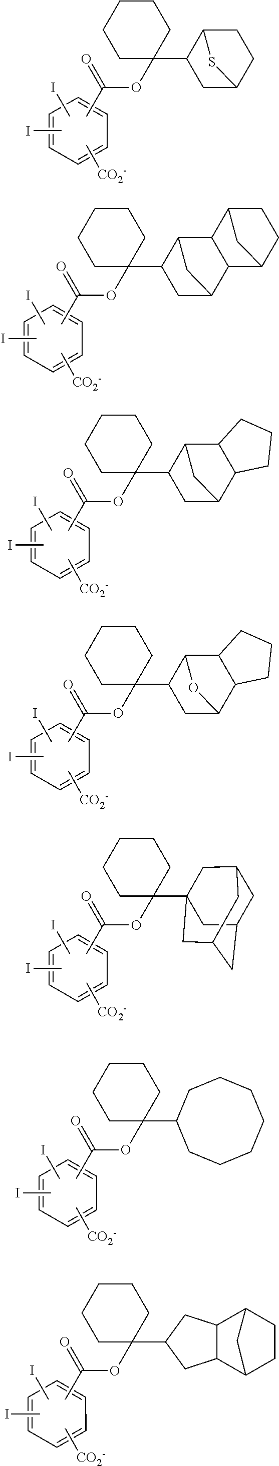

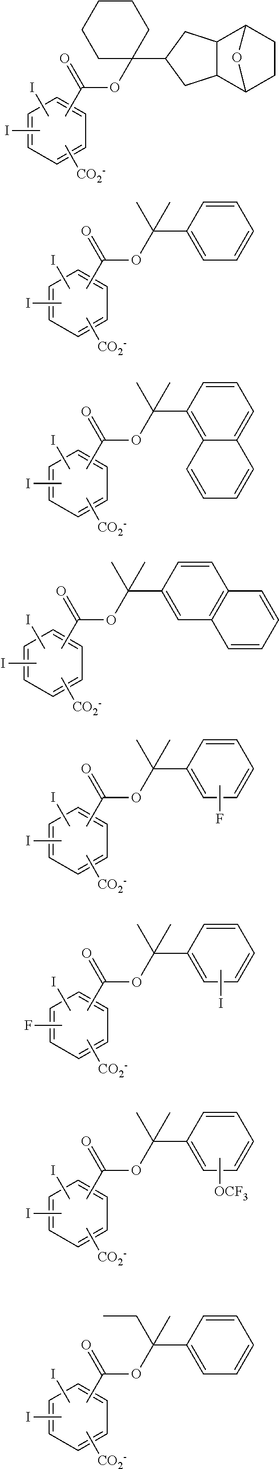

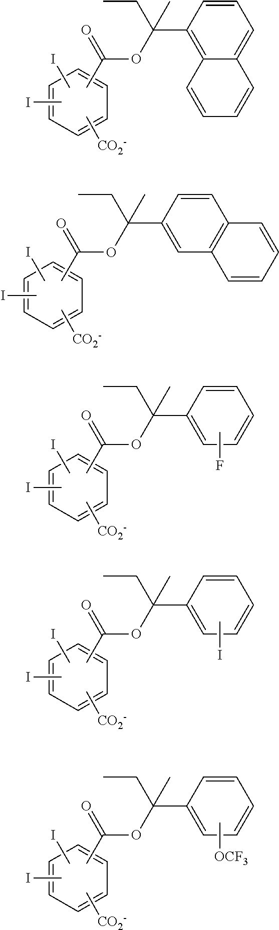

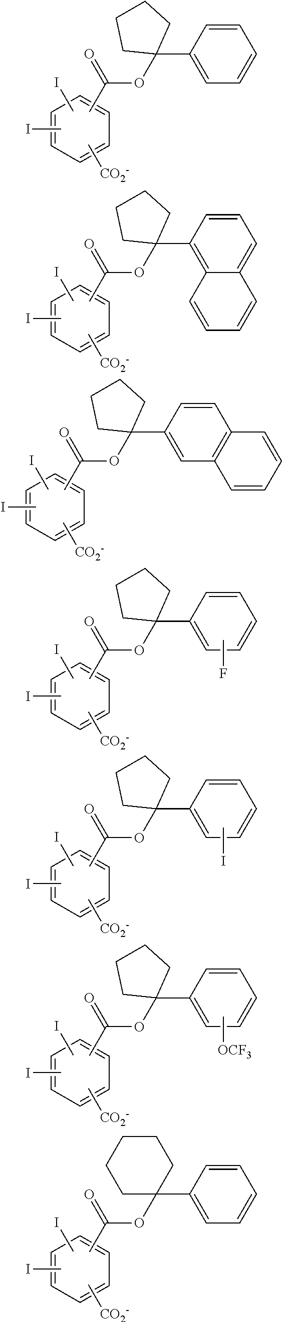

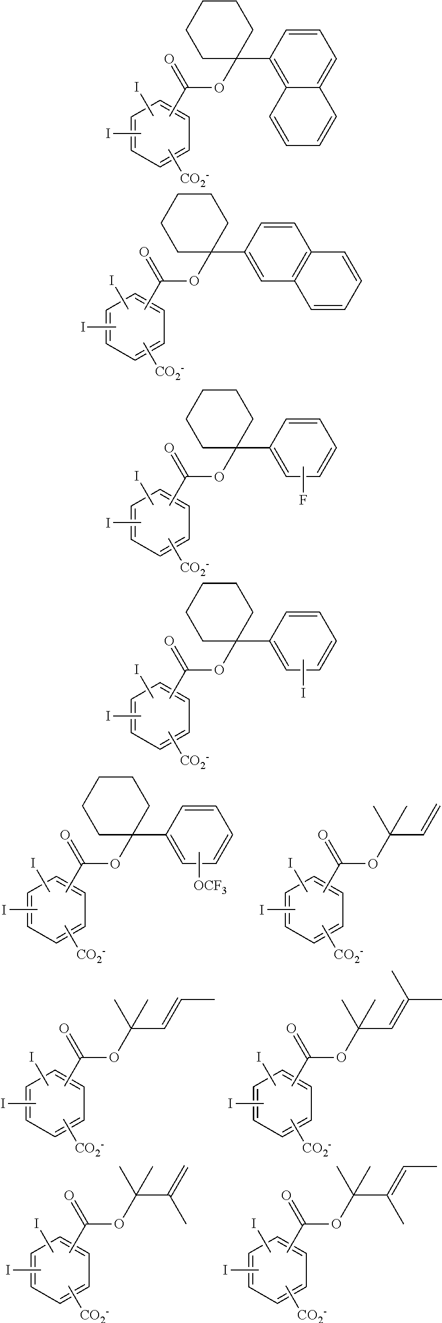

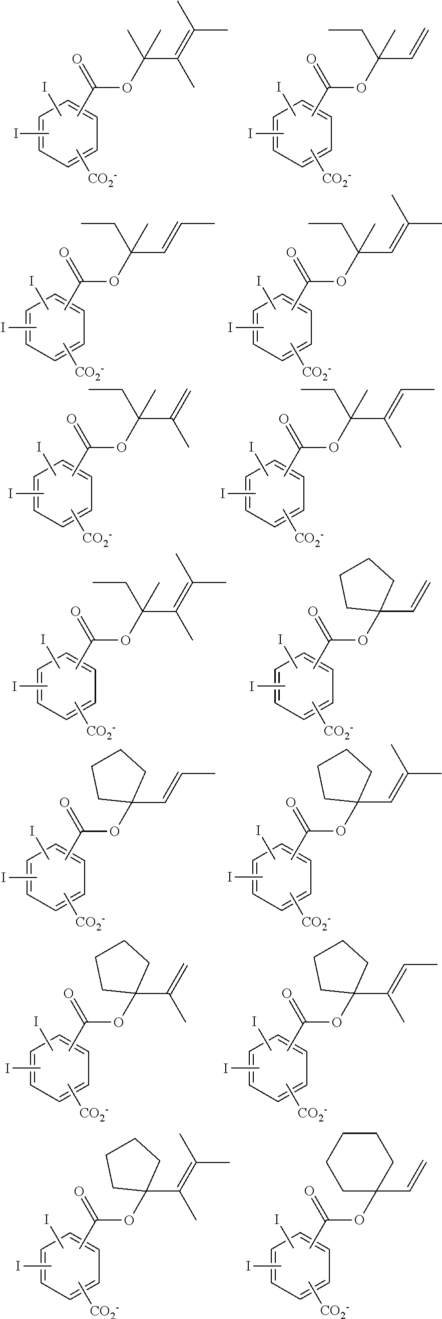

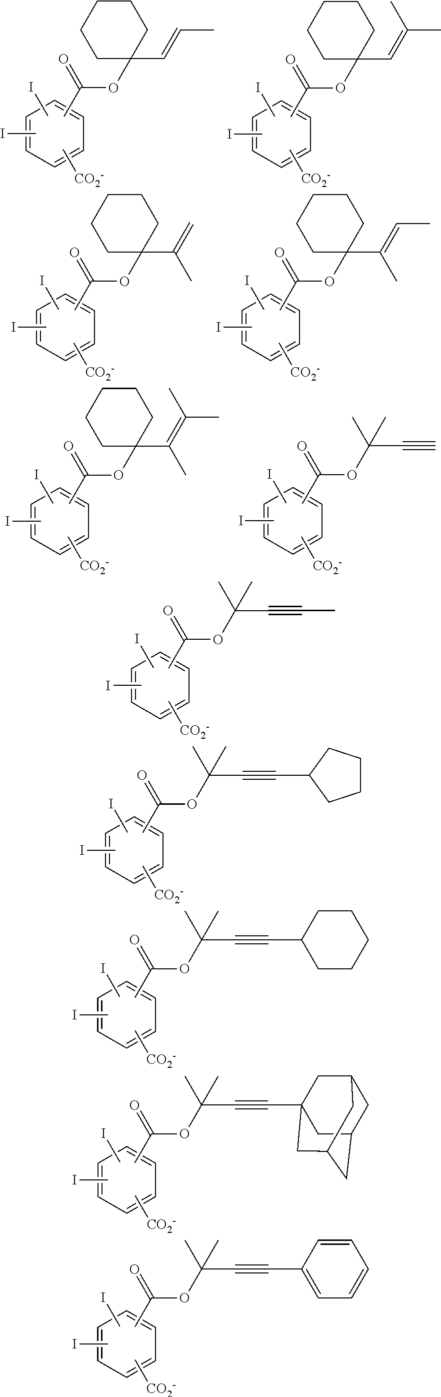

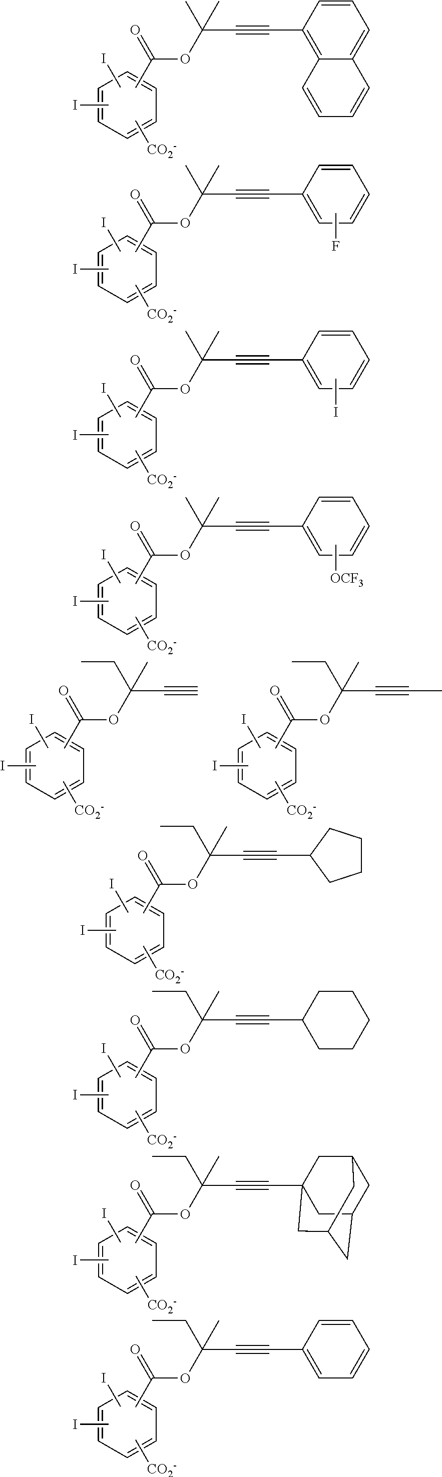

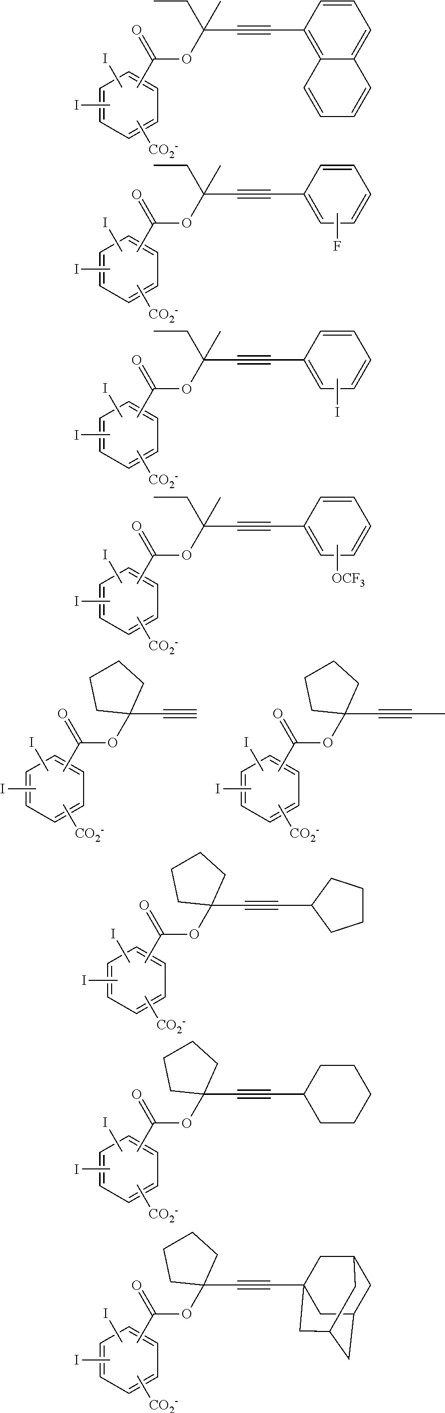

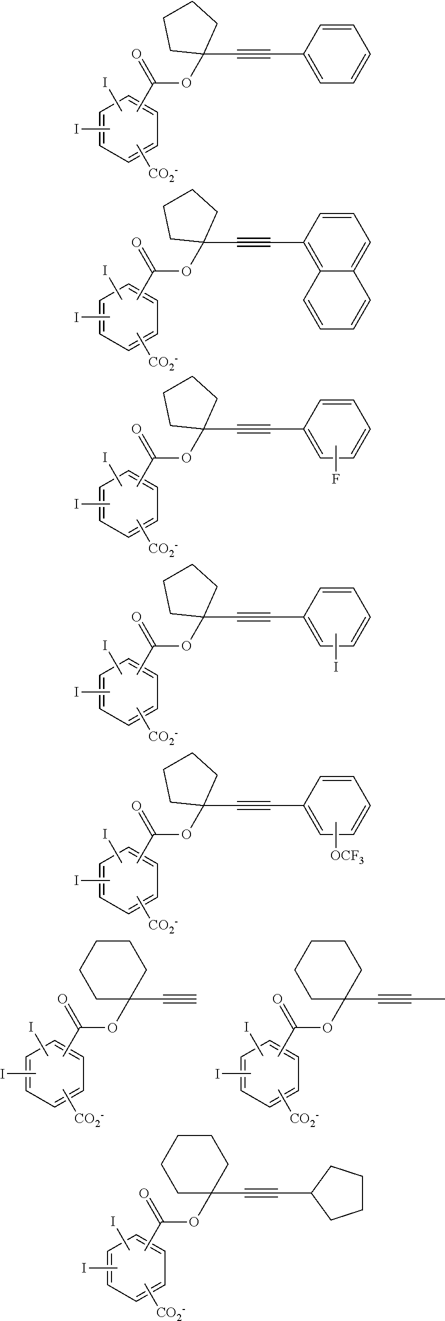

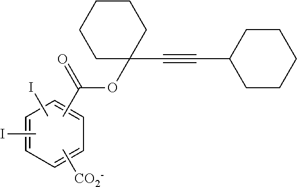

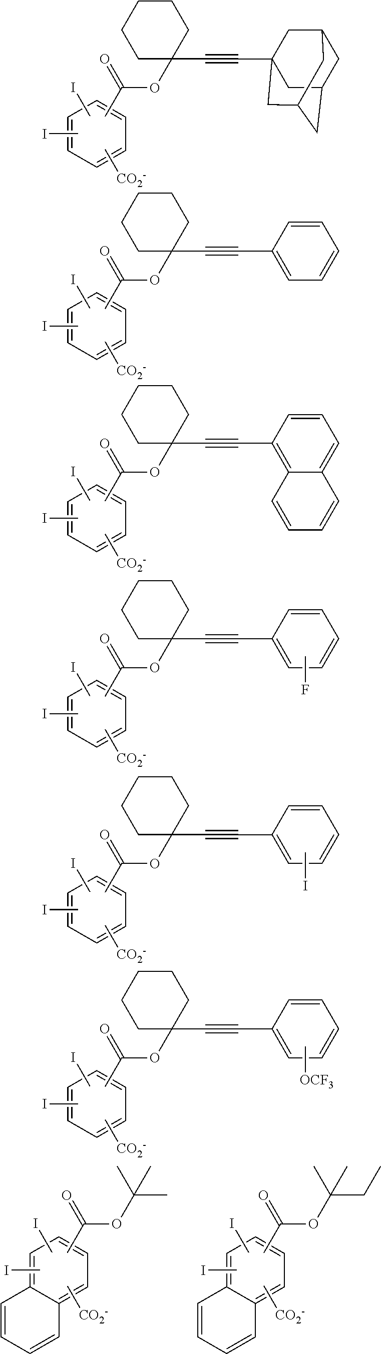

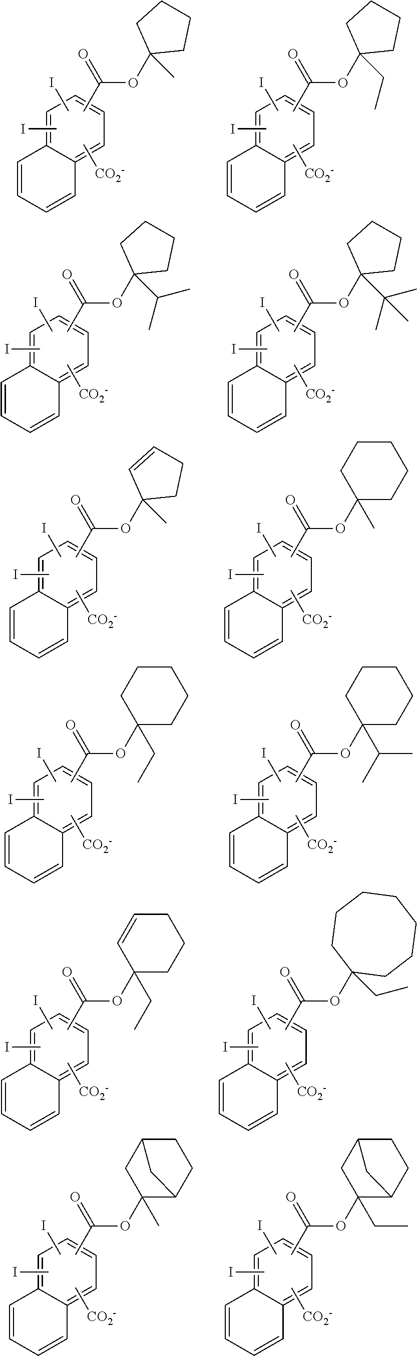

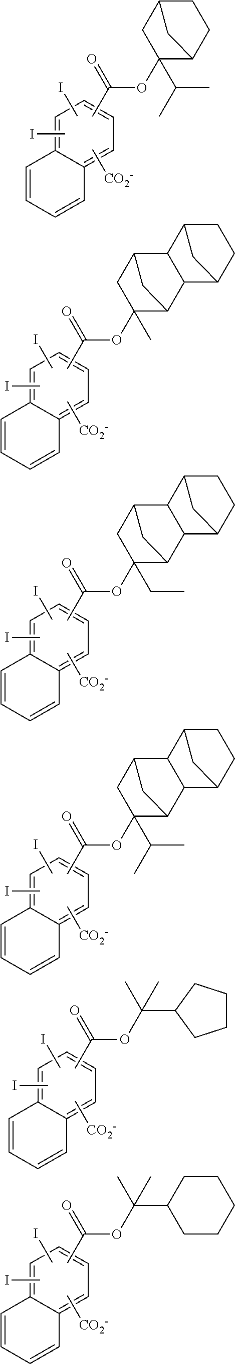

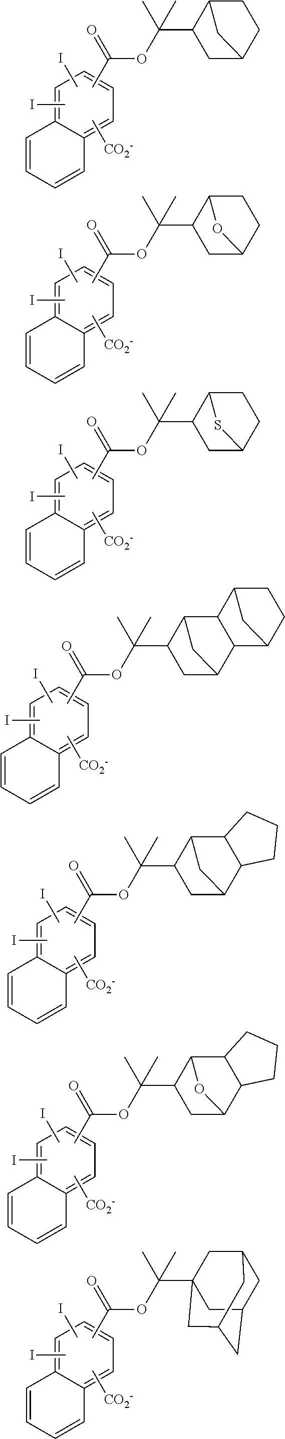

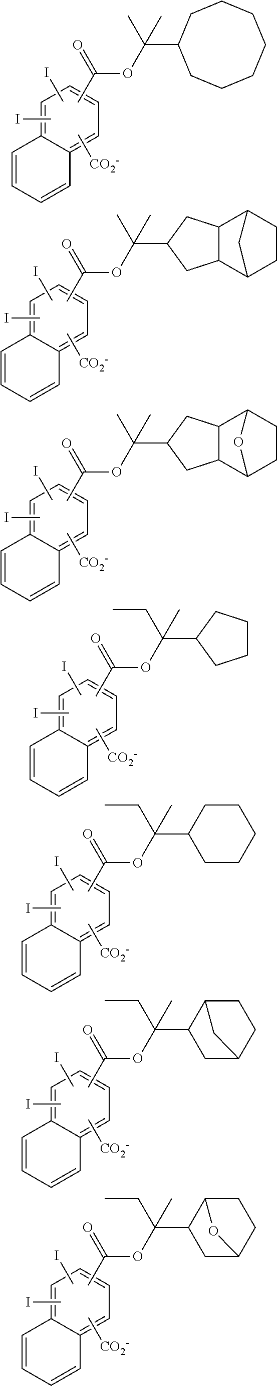

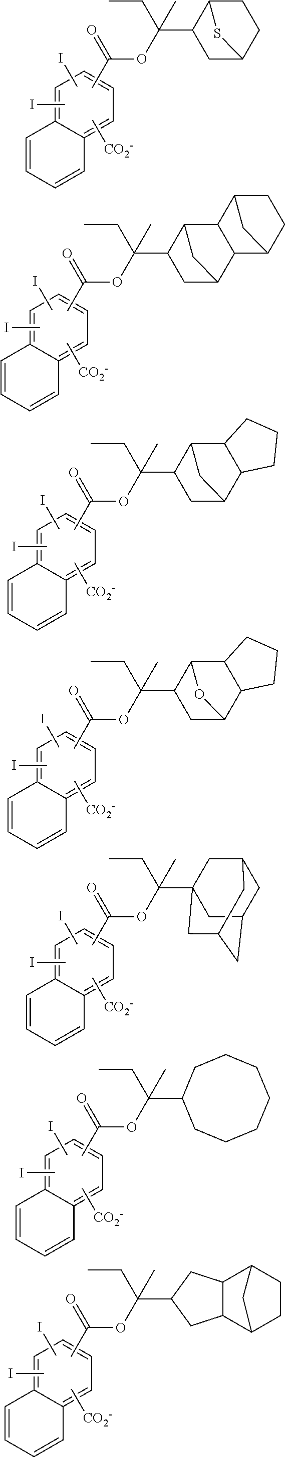

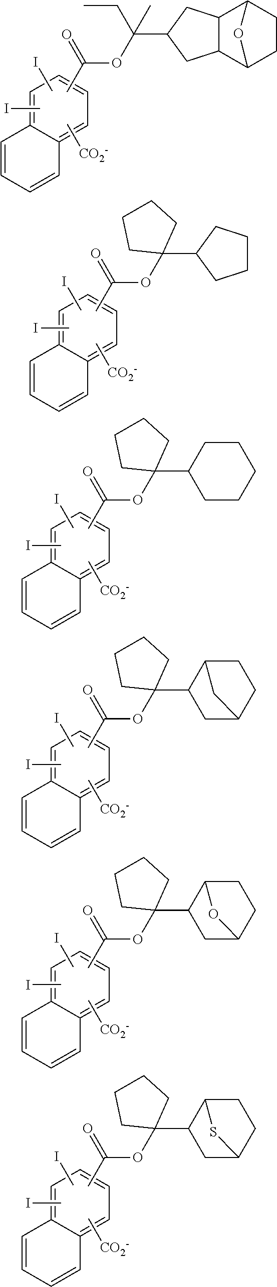

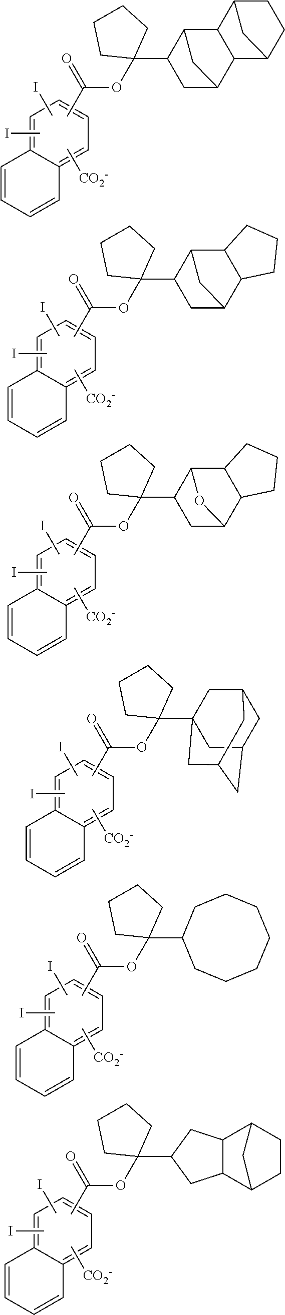

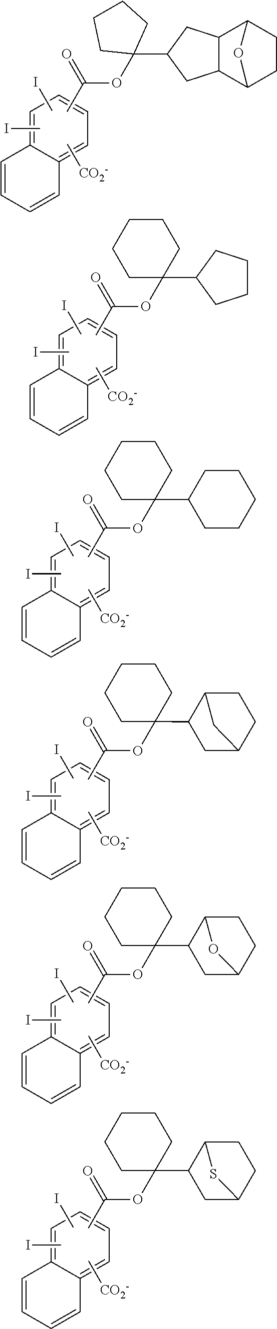

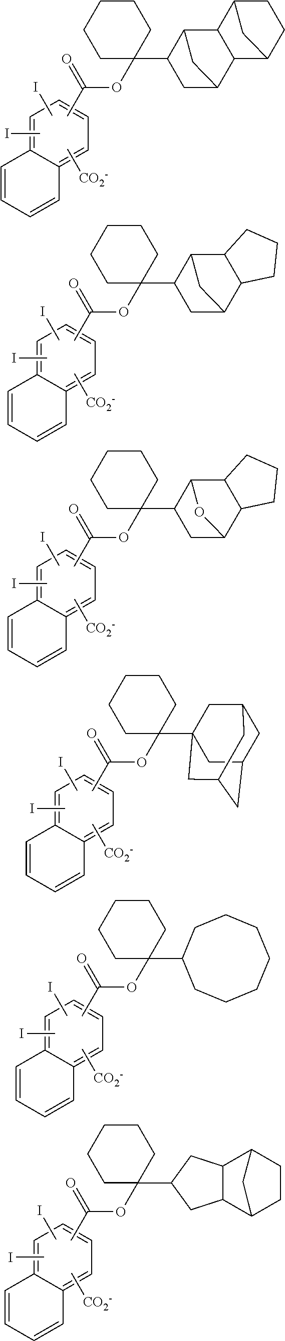

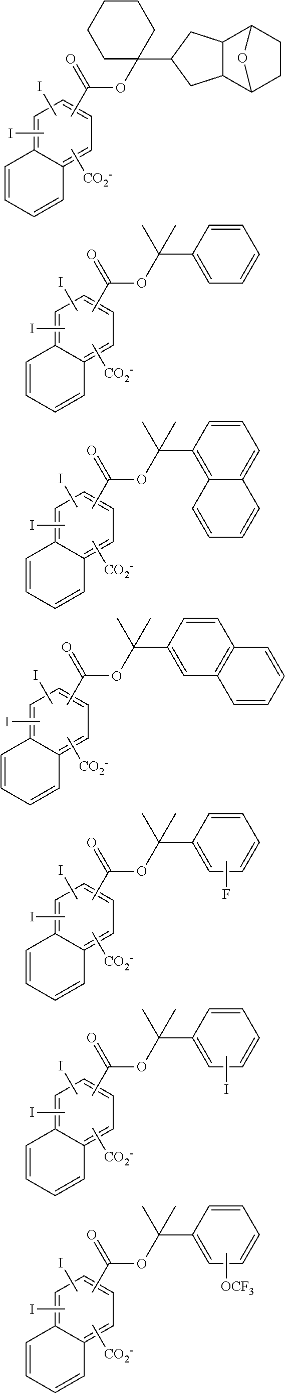

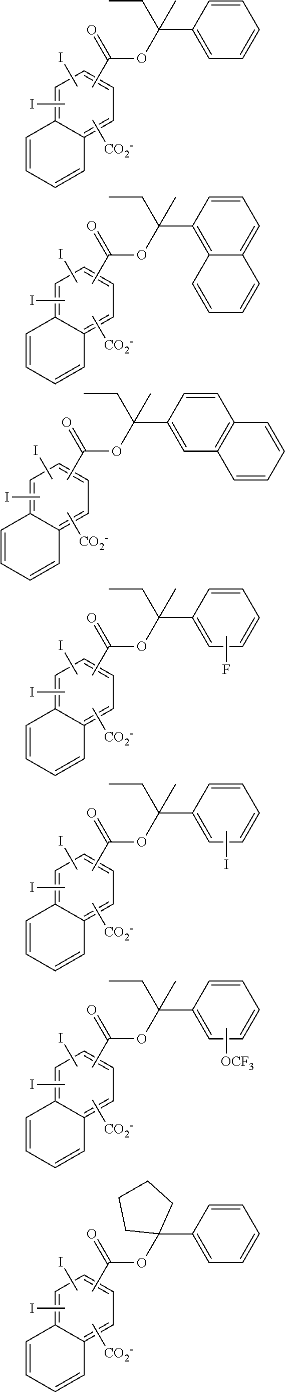

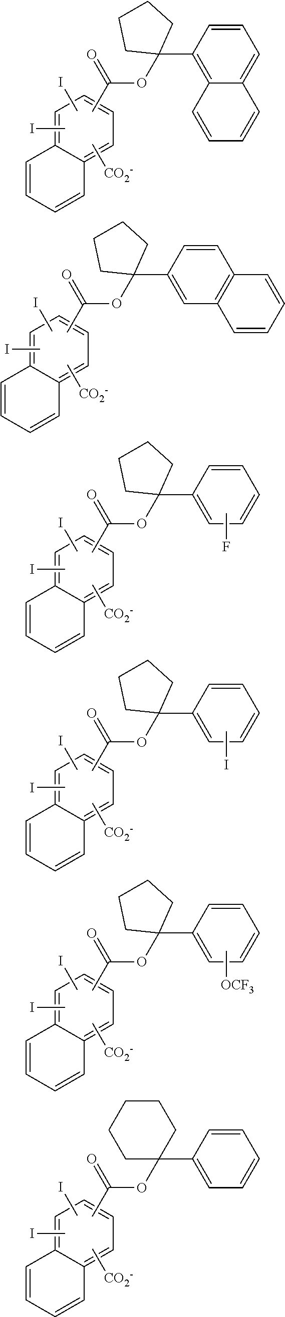

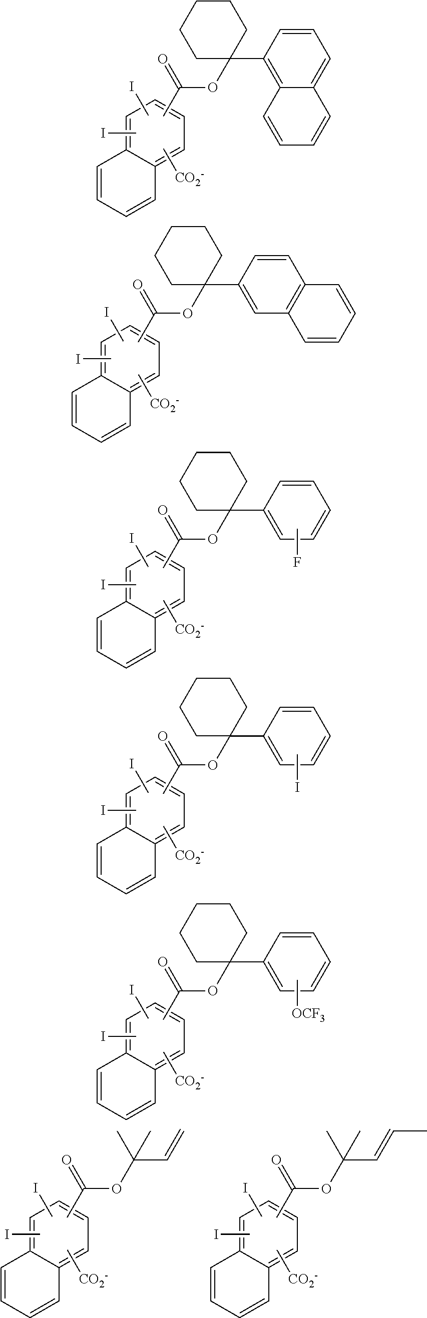

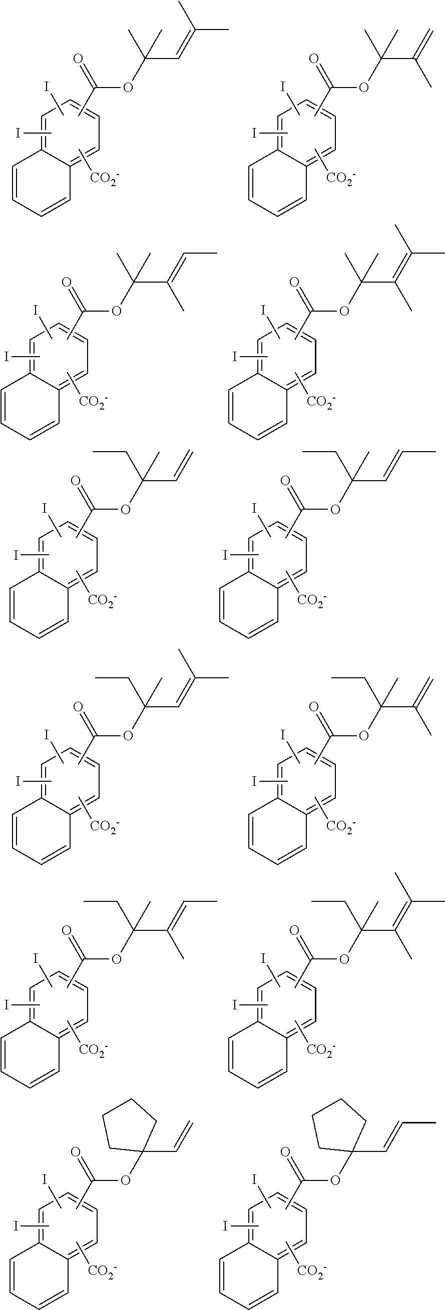

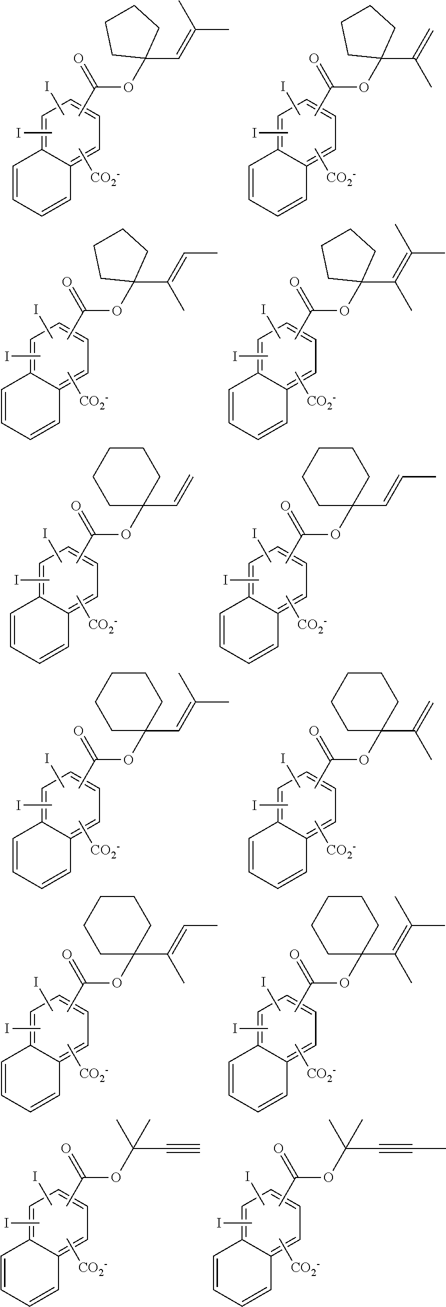

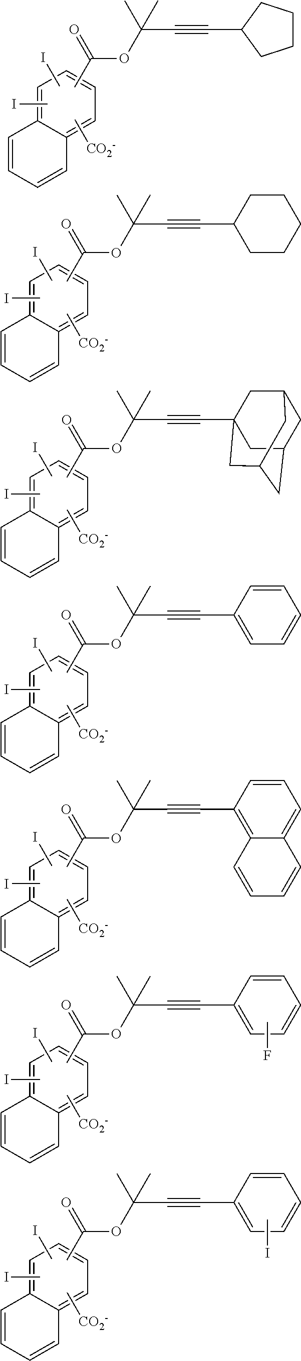

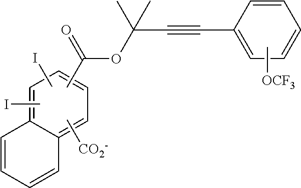

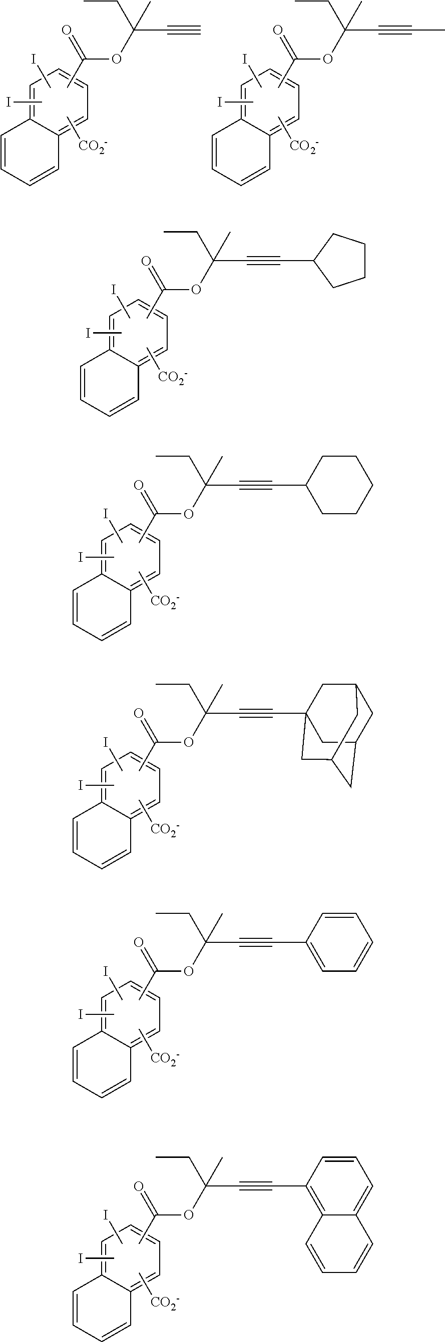

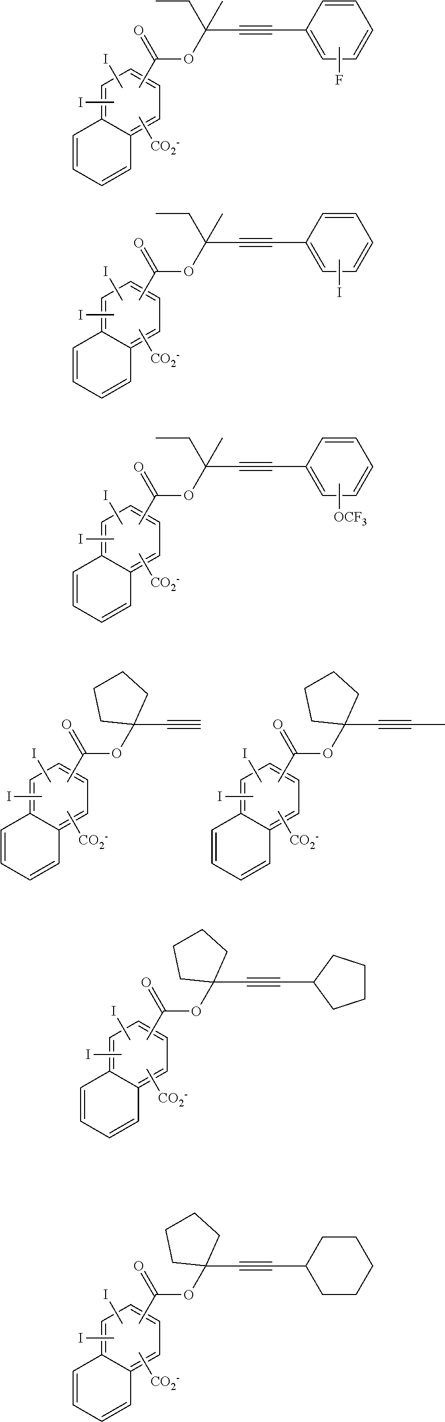

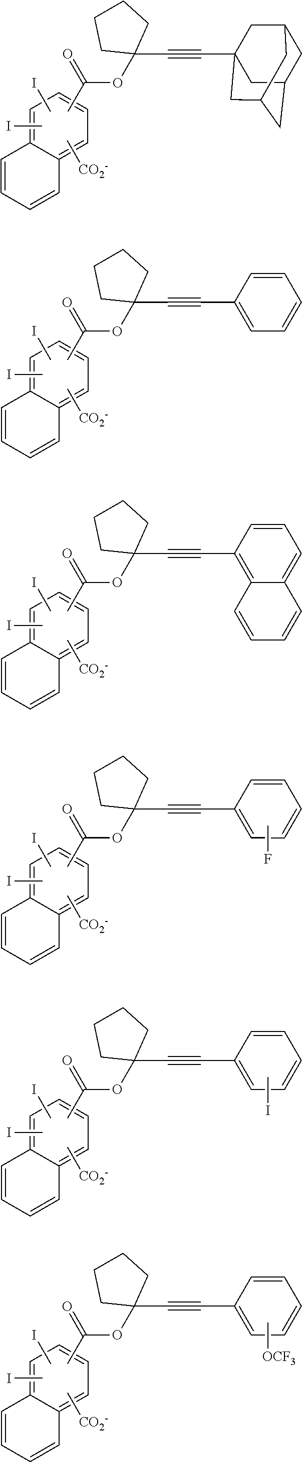

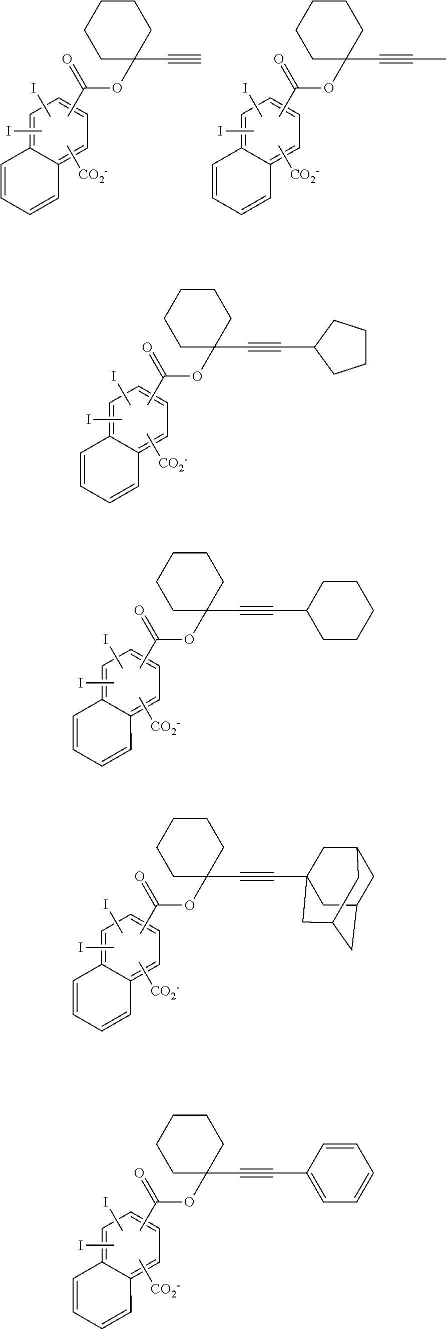

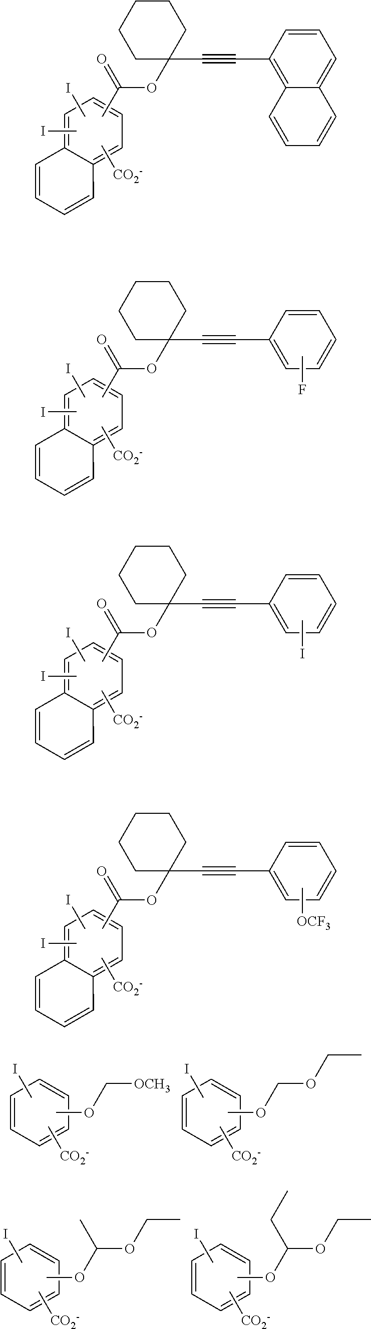

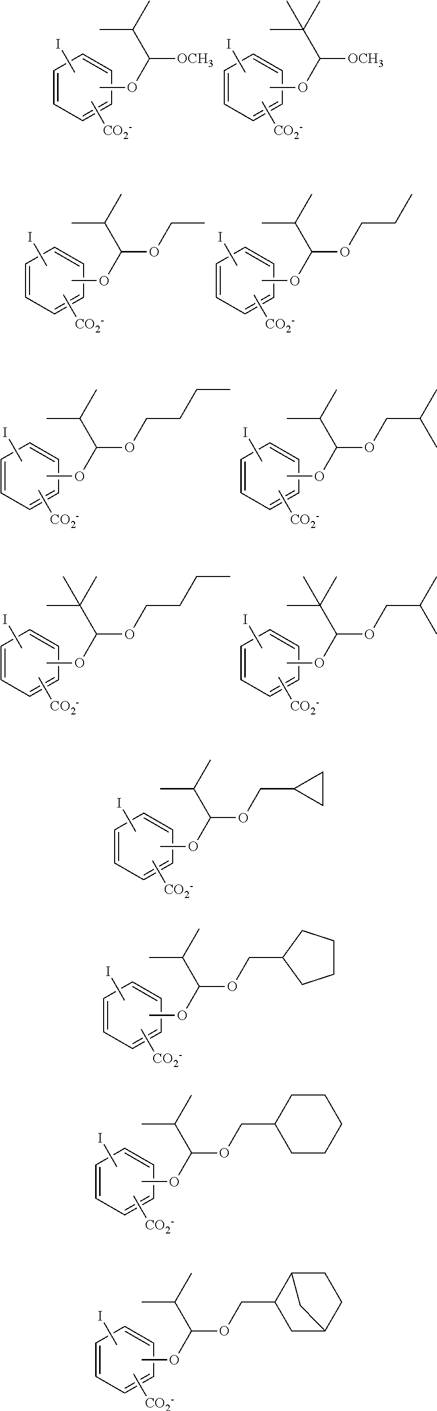

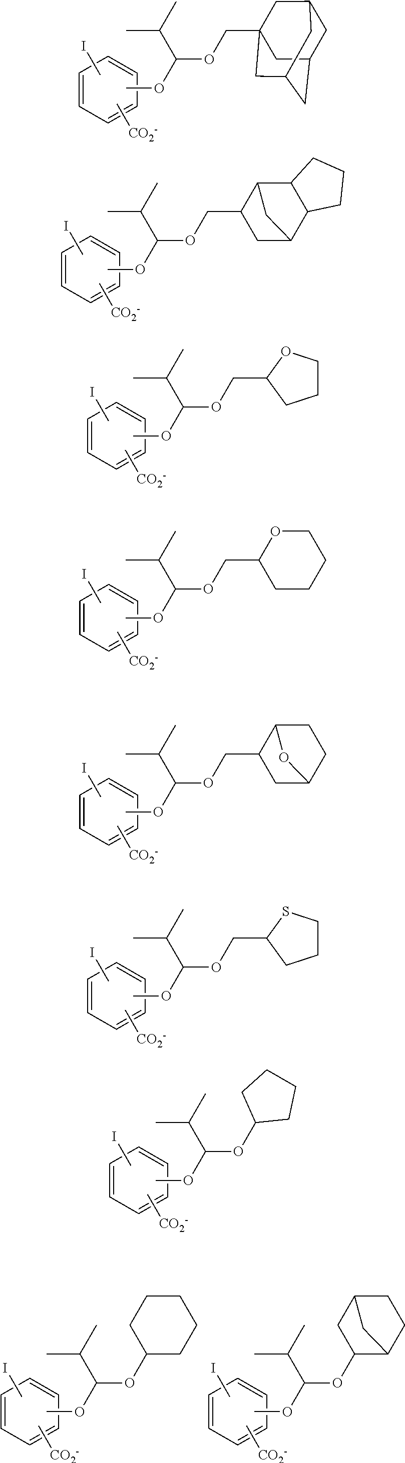

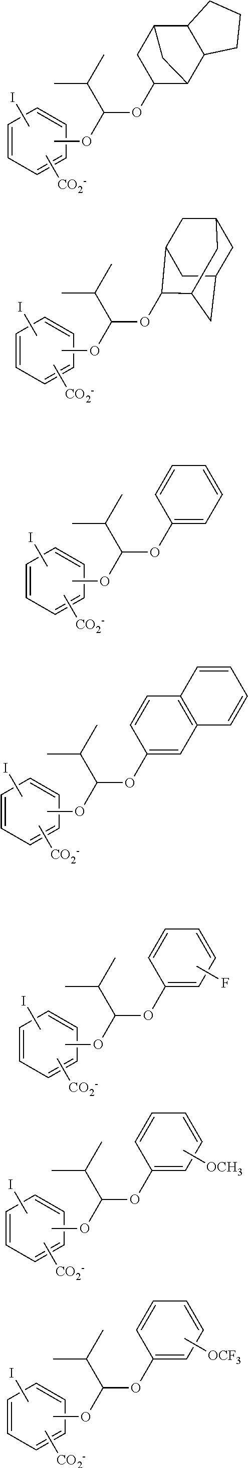

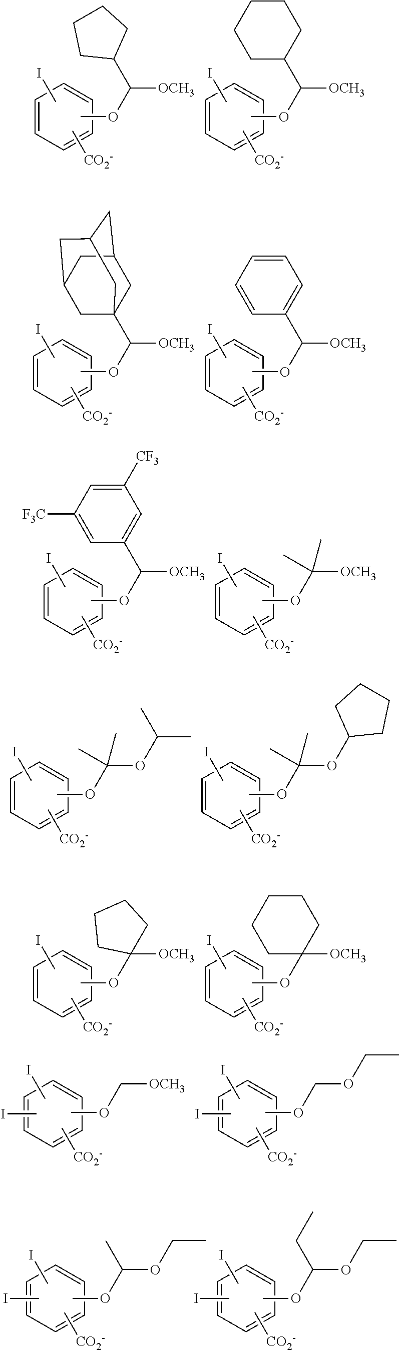

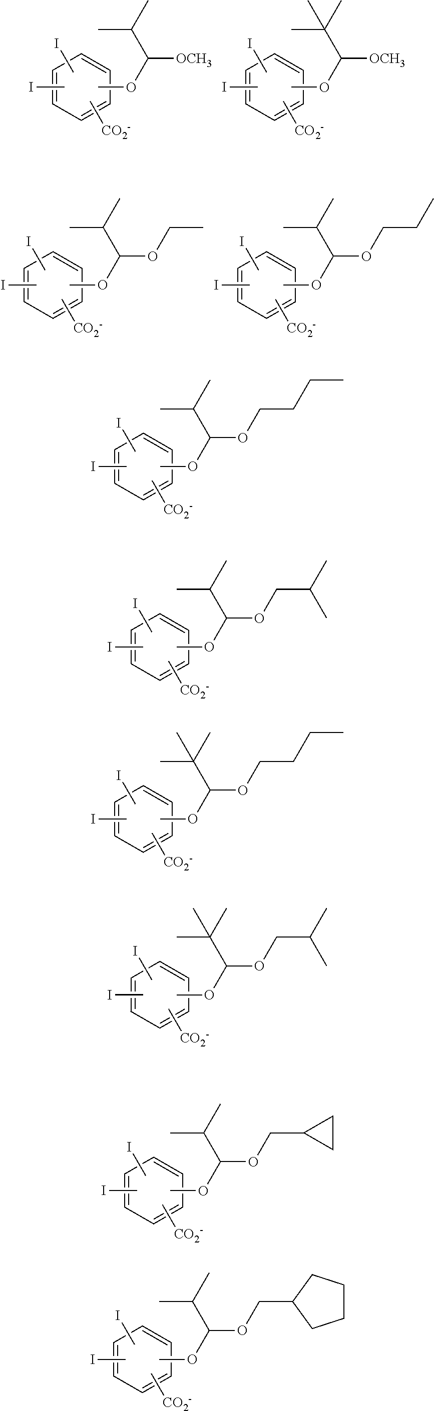

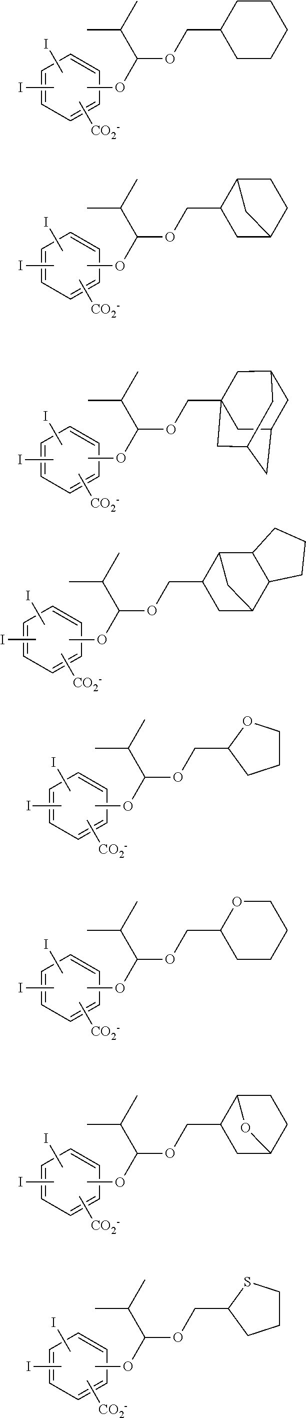

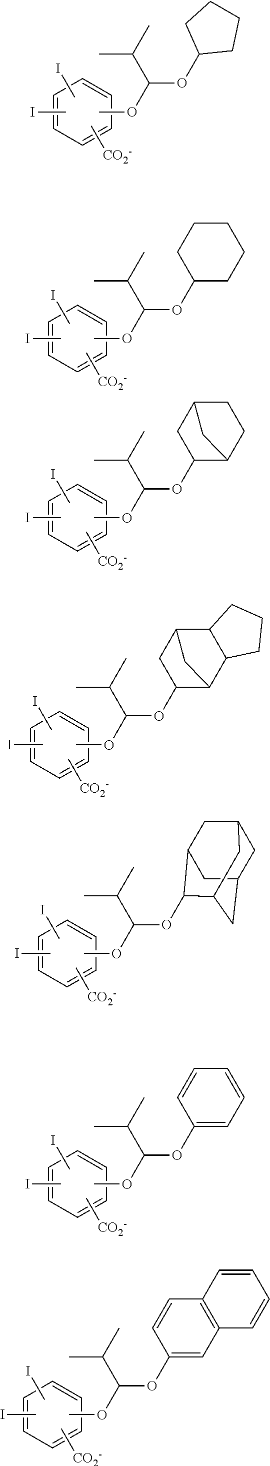

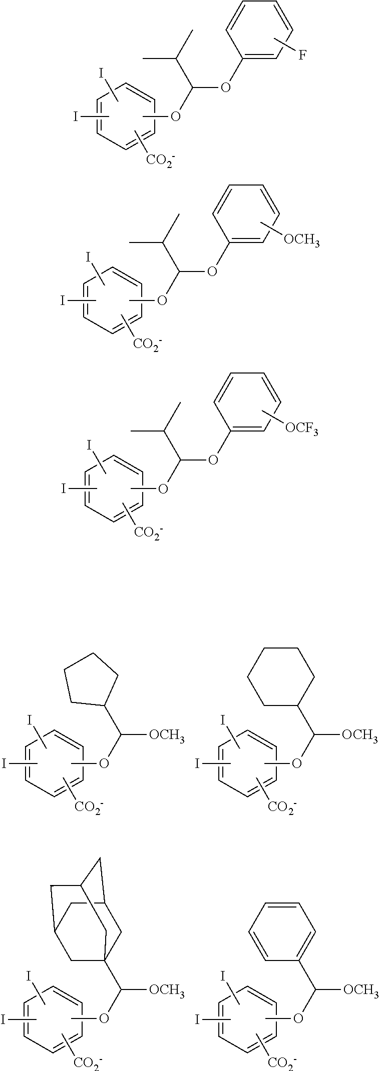

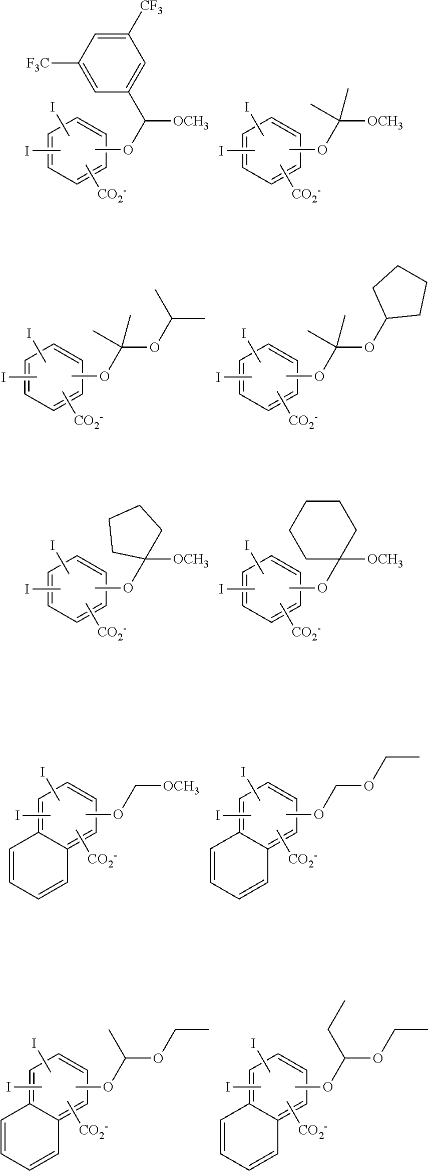

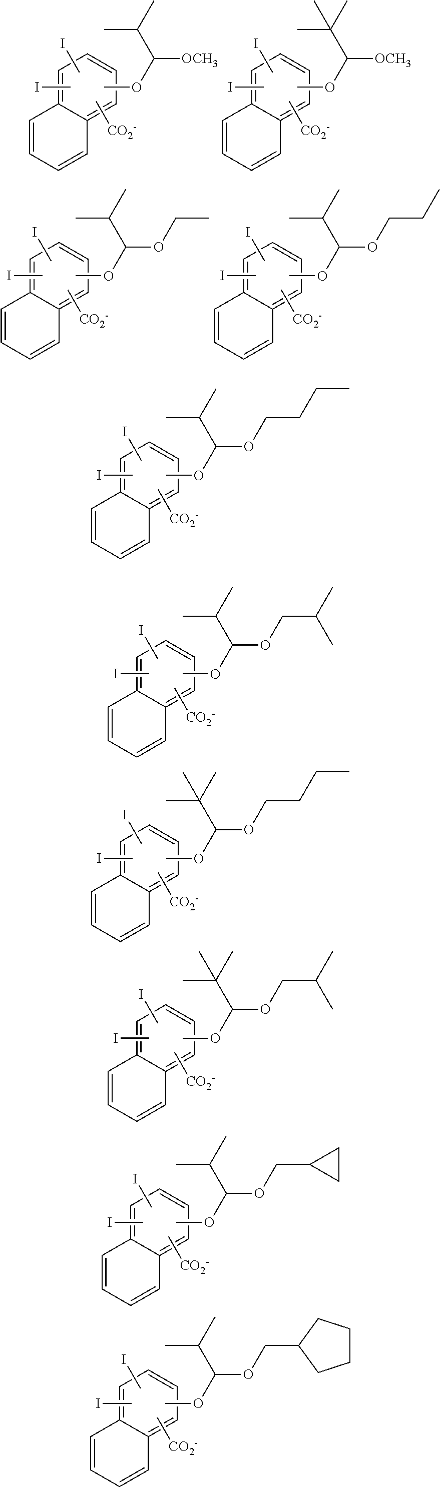

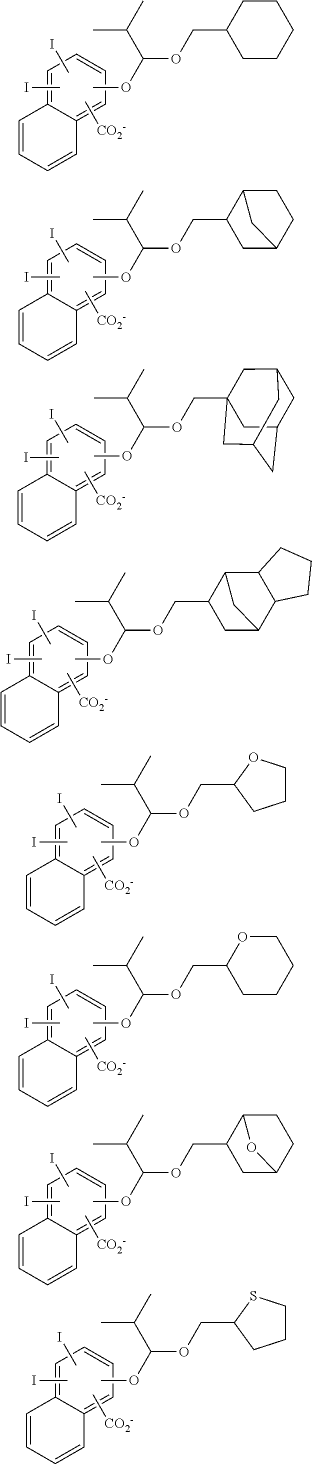

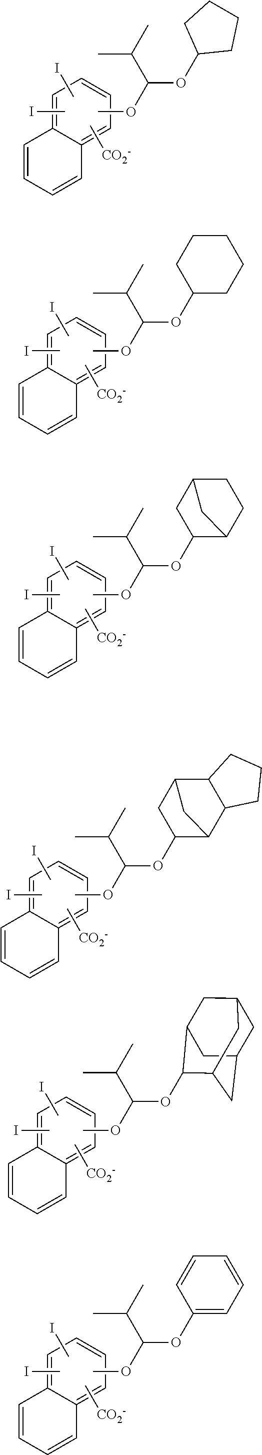

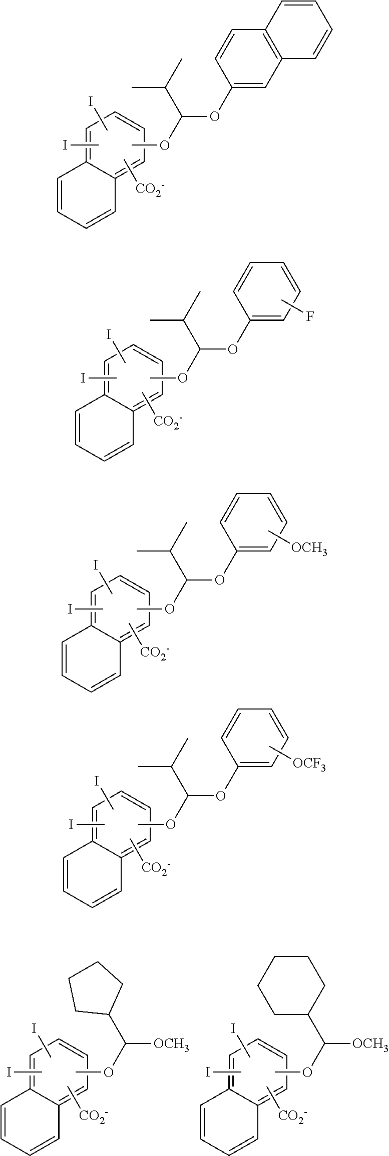

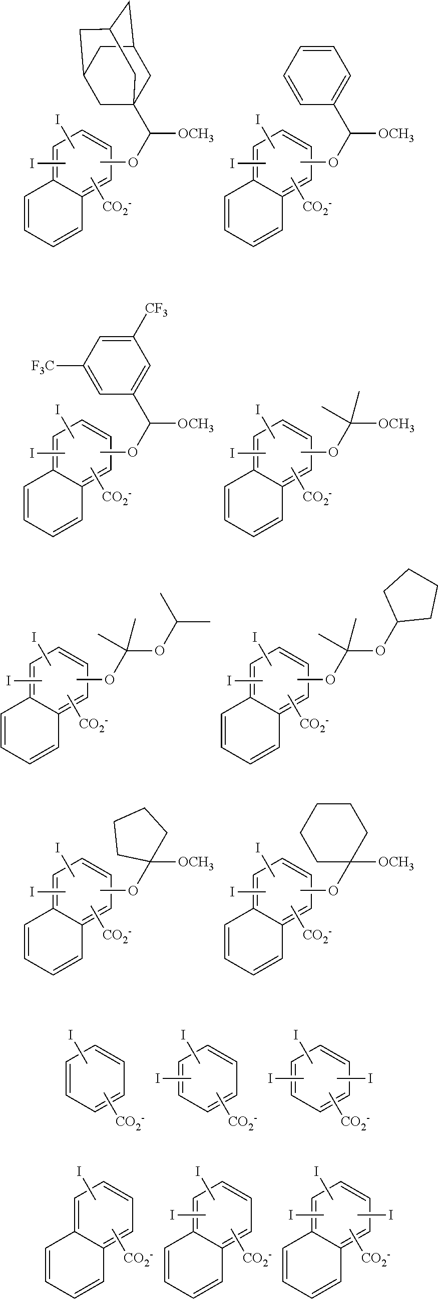

Examples of the sulfonium salt include arbitrary combinations of anions with cations, both as exemplified above.

The sulfonium salt can be synthesized by the same method as described in JP-A 2020-091312 (U.S. Pat. No. 11,586,111) although the synthesis method is not limited thereto.

The sulfonium salt of the invention is structurally characterized as consisting of a sulfonium cation containing a pentafluorosulfanyl group and an acid labile group of tertiary/secondary ether structure, tertiary/secondary carbonate structure or acetal structure and an aromatic carboxylic acid anion which may have a substituent. The substituent on the aromatic carboxylic acid anion is preferably iodine. When the iodine-containing aromatic carboxylic acid anion is processed by the EUV lithography of wavelength 13.5 nm, iodine which is outstandingly absorptive to EUV generates secondary electrons during EUV exposure. Also, iodine has an adequate dissolution-inhibiting ability to prevent the unexposed region of the resist pattern from collapsing in alkaline developer. The pentafluorosulfanyl group attached to the aromatic ring of the sulfonium cation is known to exert a strong electron-withdrawing effect. It is believed that the electron withdrawing effect lowers the energy level of the lowest unoccupied molecular orbital (LUMO) of the frontier orbital theory. Then the cation is more likely to accept the generated secondary electrons, whereby the decomposition of the cation is promoted. The synergy of these effects results in a high sensitivity.

The acid labile group of tertiary/secondary ether, tertiary/secondary carbonate or acetal structure substituting on the aromatic ring of the sulfonium cation undergoes deprotection reaction by the acid generated upon light exposure, creating a corresponding phenol. The structural change from lipophilic to hydrophilic one increases the contrast between exposed and unexposed regions. Also, since the pentafluorosulfanyl group is attached to the carbon atom adjoining the carbon atom to which the thus created phenolic hydroxy group is attached, the electron-withdrawing effect of pentafluorosulfanyl group serves to increase the acidity of the phenol. When the exposed region where deprotection reaction has taken place is developed in an alkaline developer, the phenol having the increased acidity serves to improve the solubility in alkaline developer. Accordingly, development residues are minimized, and any swell in alkaline developer is suppressed to prevent the resist pattern from collapsing. In an embodiment wherein the anion also contains an acid labile group, deprotection reaction of this acid labile group takes place with the aid of the generated acid to bring about a further change of solubility, from which an enhancement of contrast and a higher sensitivity are expectable. In the exposed region of the positive resist film, as the affinity to alkaline developer is higher, the risk of development defects is lowered. The sulfonium salt having an aromatic carboxylic acid anion has a relatively strong basicity whereby the strong acid generated from the PAG is effectively trapped. Due to the synergy of these effects, a chemically amplified resist composition comprising the inventive sulfonium salt exhibits a high dissolution contrast and can form line patterns with reduced LWR and hole patterns with improved CDU, both the patterns having collapse resistance. The inventive sulfonium salt is thus useful as one component of a chemically amplified resist composition, especially of positive tone.

The inventive sulfonium salt is advantageously used as a quencher. As used herein, the term “quencher” refers to a compound capable of trapping the strong acid generated by a PAG in the resist composition to prevent the acid from diffusing to the unexposed region and to assist in forming the desired pattern. The term “PAG” refers to a compound capable of generating a strong acid upon exposure to high-energy radiation. The term “strong acid” refers to a compound having a sufficient acidity to induce deprotection reaction of an acid labile group. Since the aromatic carboxylic acid does not have an enough acidity to induce deprotection reaction of an acid labile group which is tertiary ester or tertiary ether, it is effective to separately add a PAG capable of generating a strong acid, i.e., α-fluorinated sulfonic acid, imide acid or methide acid, for the purpose of inducing deprotection reaction of an acid labile group, as will be described later. It is noted that the PAG capable of generating an α-fluorinated sulfonic acid, imide acid or methide acid may be of addition type or of polymer-bound type wherein the PAG is bound to a base polymer.

In a system where the inventive sulfonium salt capable of generating an aromatic carboxylic acid and a PAG capable of generating a ultra-strong acid or perfluoroalkylsulfonic acid are co-present, the aromatic carboxylic acid and the perfluoroalkylsulfonic acid generate upon light exposure. Since the PAG is not decomposed in its entirety, some PAG remains undecomposed nearby. If the sulfonium salt capable of generating an aromatic carboxylic acid and perfluoroalkylsulfonic acid are co-present at this point of time, first an ion exchange occurs between the perfluoroalkylsulfonic acid and the onium salt capable of generating an aromatic carboxylic acid whereby an onium salt of perfluoroalkylsulfonic acid is generated and the aromatic carboxylic acid is released. This is because the perfluoroalkylsulfonic acid salt having a high acid strength is more stable. On the other hand, where the perfluoroalkylsulfonic acid sulfonium salt and the aromatic carboxylic acid are co-present, no ion exchange occurs. Similar ion exchange takes place not only with the perfluoroalkylsulfonic acid, but also with an arene sulfonic acid, alkyl sulfonic acid, imide acid or methide acid having a higher acid strength than the aromatic carboxylic acid generated by the inventive sulfonium salt.

### [Chemically Amplified Resist Composition]

### (A) Quencher

Another embodiment of the invention is a chemically amplified resist composition essentially comprising (A) a quencher in the form of the sulfonium salt having formula (1).

In the chemically amplified resist composition, the amount of quencher (A) is preferably 0.1 to 40 parts by weight, more preferably 0.5 to 30 parts by weight per 80 parts by weight of a base polymer to be described just below. As long as the amount of quencher (A) is in the range, good sensitivity and resolution are achievable and the risk of foreign particles being formed after development or during stripping of resist film is avoided. The quencher may be used alone or in admixture.

### (B) Base Polymer

The chemically amplified resist composition may comprise a base polymer as component (B). The base polymer (B) preferably comprises repeat units having an acid labile group. The repeat units having an acid labile group may be used alone or in admixture of two or more types.

The preferred repeat units having an acid labile group are repeat units having the formula (a1) and repeat units having the formula (a2), which are also referred to as repeat units (a1) and (a2).

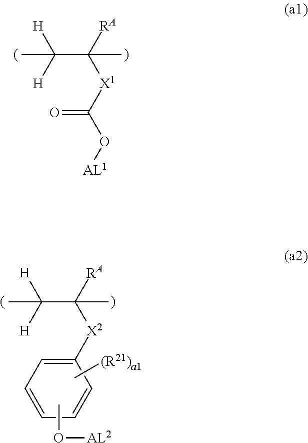

In formulae (a1) and (a2), R^{A} is hydrogen, fluorine, methyl or trifluoromethyl.

In formula (a1), X¹ is a single bond, phenylene group, naphthylene group, *—C(═O)—O—X¹¹— or *—C(═O)—N(H)—X¹¹—. The phenylene or naphthylene group may be substituted with hydroxy, nitro, cyano, an optionally fluorinated C₁-C₁₀ saturated hydrocarbyl moiety, optionally fluorinated C₁-C₁₀ saturated hydrocarbyloxy moiety, or halogen. X¹¹ is a C₁-C₁₀ saturated hydrocarbylene group, phenylene or naphthylene group, and the saturated hydrocarbylene group may contain a hydroxy moiety, ether bond, ester bond or lactone ring. The asterisk (*) designates a point of attachment to the carbon atom in the backbone.

In formula (a2), X² is a single bond or *—C(═O)—O— wherein * designates a point of attachment to the carbon atom in the backbone. R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. The subscript a1 is 0, 1, 2, 3 or 4, preferably 0 or 1. When a1 is 2, 3 or 4, a plurality of R²¹ may be identical or different.

In formulae (a1) and (a2), AL¹ and AL² are each independently an acid labile group. Examples of the acid labile group include those described in U.S. Pat. No. 8,574,817 (JP-A 2013-080033) and U.S. Pat. No. 8,846,303 (JP-A 2013-083821).

Typical of the acid labile group are groups of the following formulae (AL-5) to (AL-7).

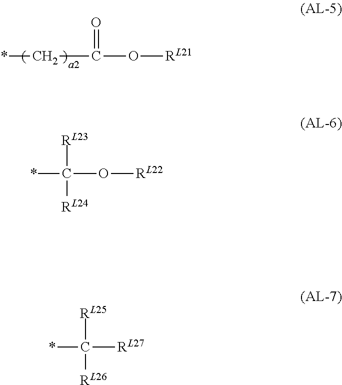

In formulae (AL-5) and (AL-6), R^{L21} and R^{L22} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ hydrocarbyl groups are preferred.

In formula (AL-5), a2 is an integer of 0 to 10, preferably 1, 2, 3, 4 or 5.

In formula (AL-6), R^{L23} and R^{L24} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L22}, R^{L23} and R^{L24} may bond together to form a C₃-C₂₀ ring with the carbon atom or carbon and oxygen atoms to which they are attached. The ring preferably contains 4 to 16 carbon atoms and is typically alicyclic.

In formula (AL-7), R^{L25}, R^{L26} and R^{L27} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L25}, R^{L26} and R^{L27} may bond together to form a C₃-C₂₀ ring with the carbon atom to which they are attached. The ring preferably contains 4 to 16 carbon atoms and is typically alicyclic.

Other examples of the acid labile group include those described in JP-A 2023-123222, paragraphs [0064]-[0068] and JP 7492842, paragraphs [0013]-[0014]. After acid elimination reaction, these acid labile groups produce conjugated olefins or acrylate derivatives which are utilized as a driving force to run the reaction forward.

Examples of repeat unit (a1) are shown below, but not limited thereto. Herein R^{A} and AL¹ are as defined above.

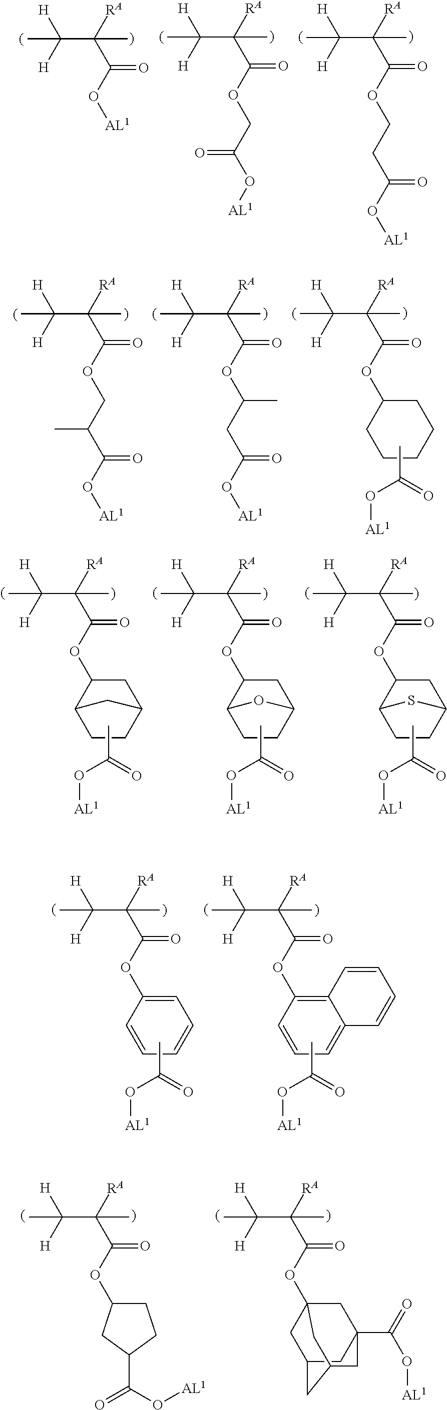

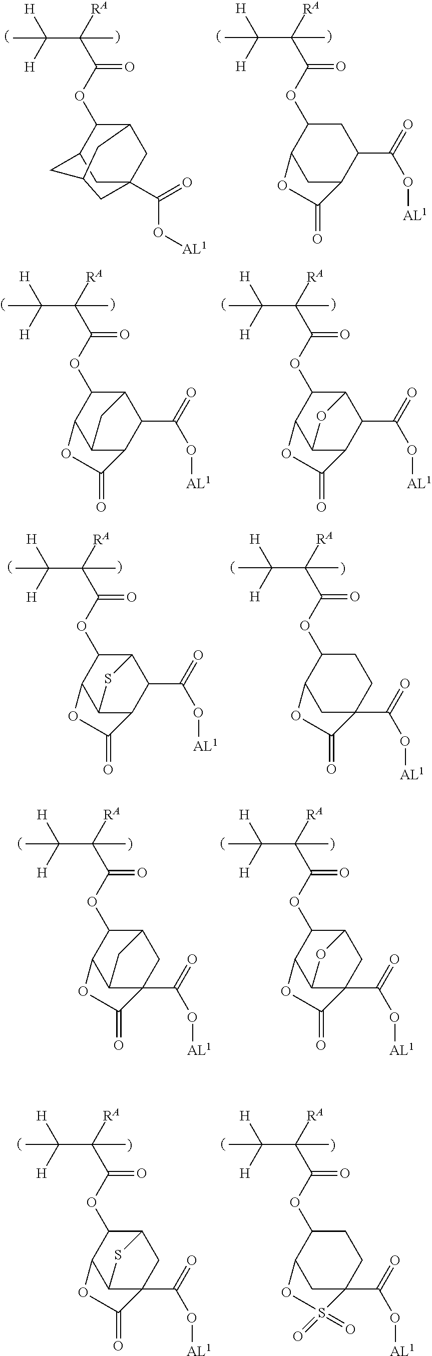

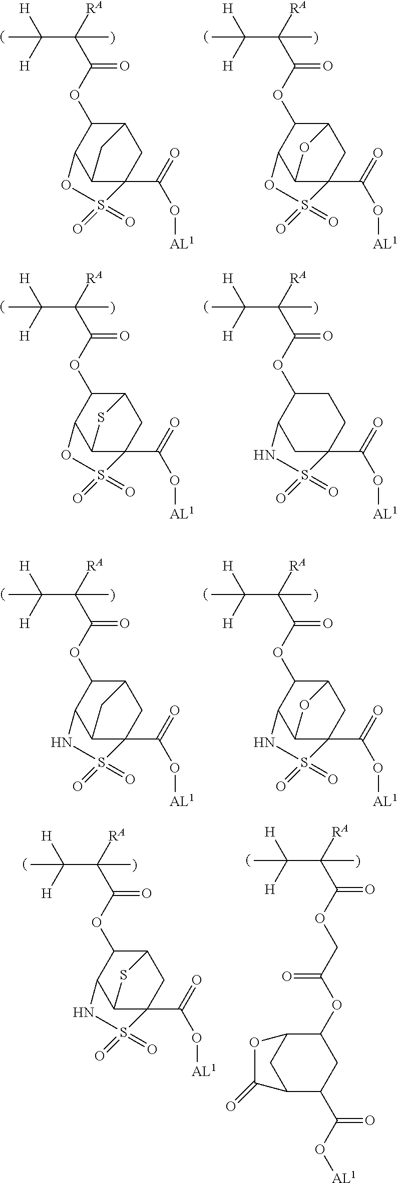

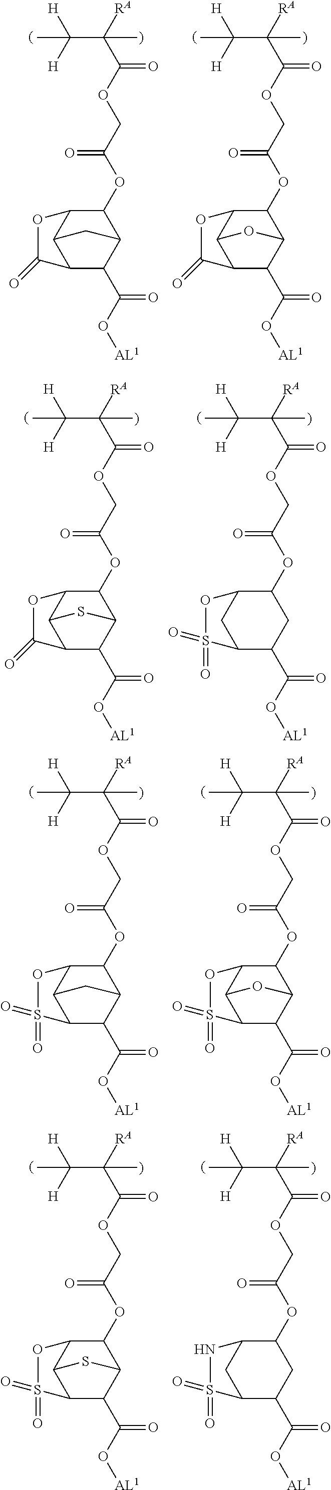

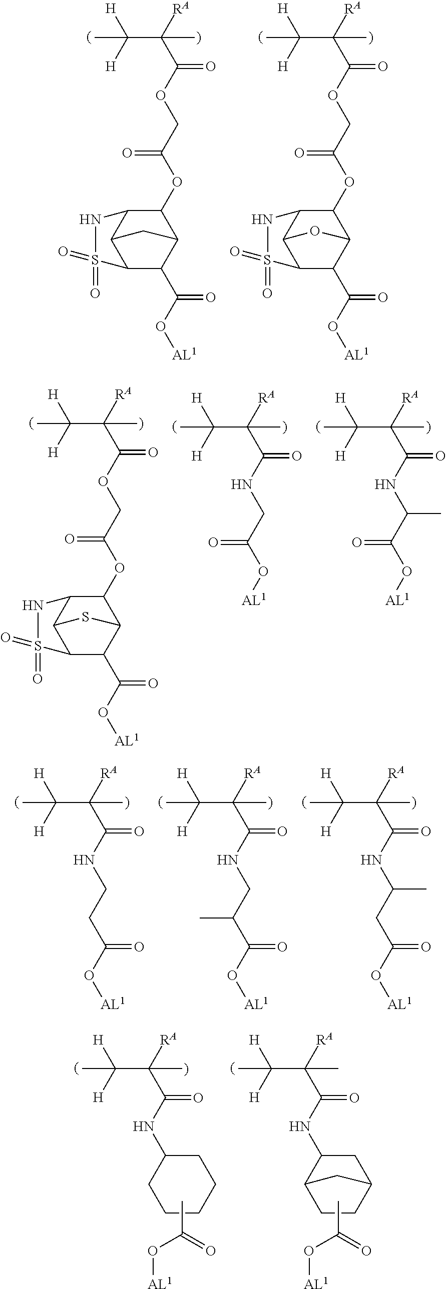

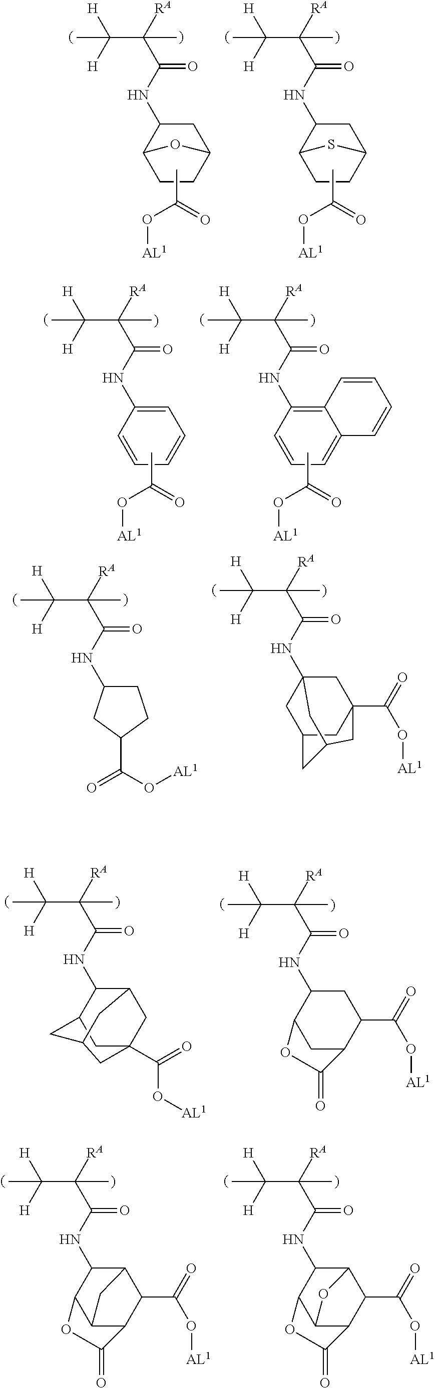

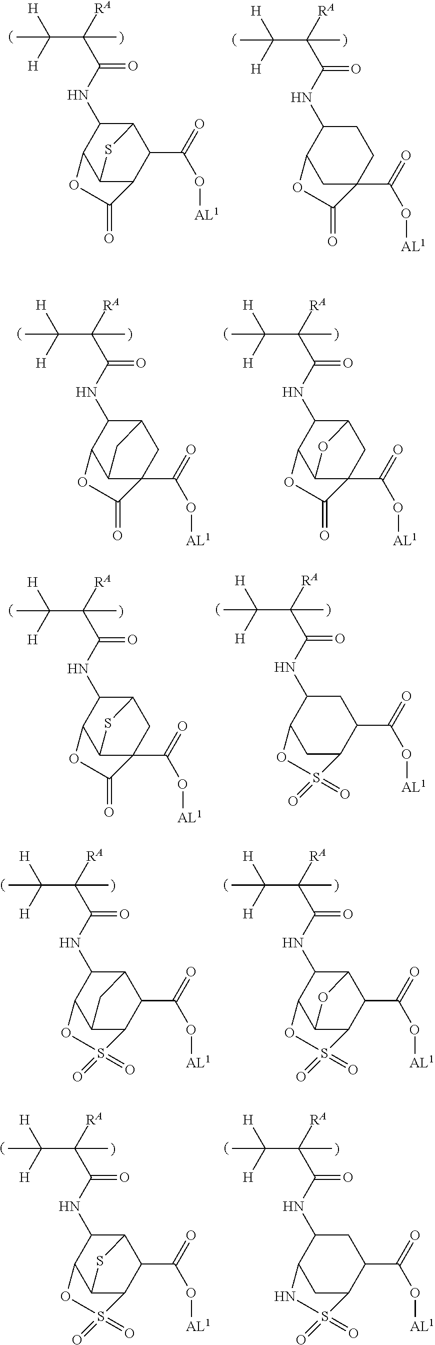

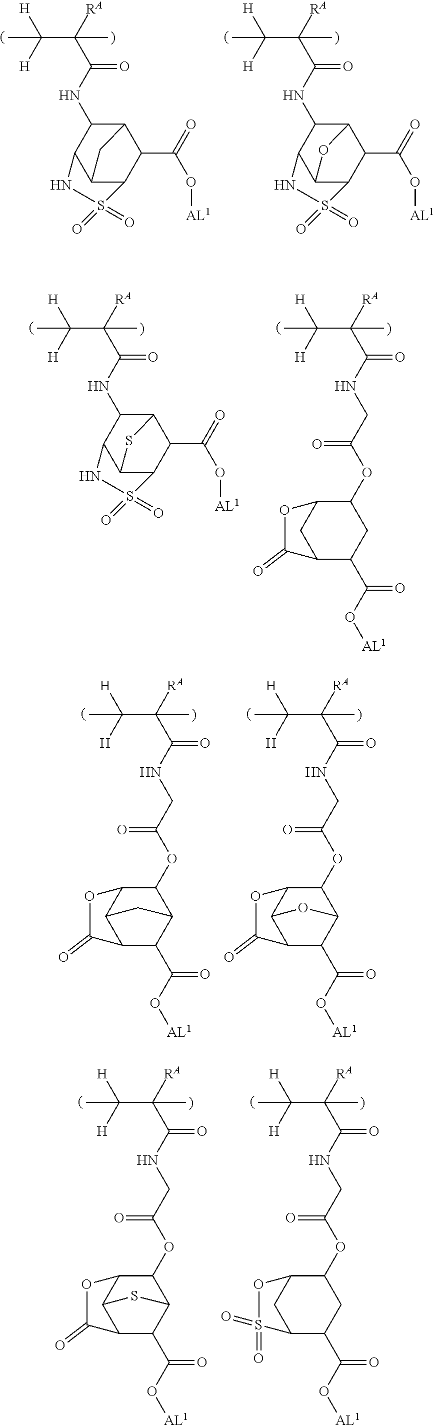

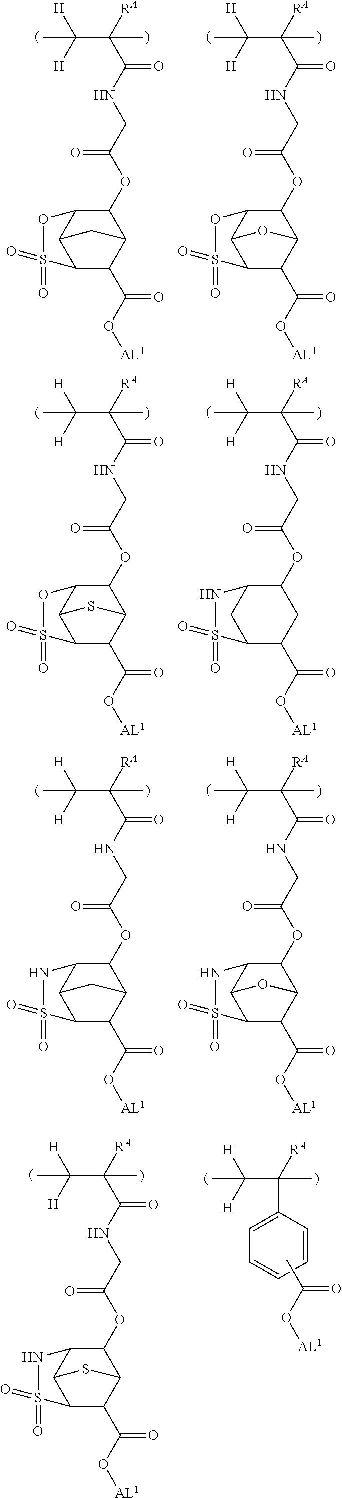

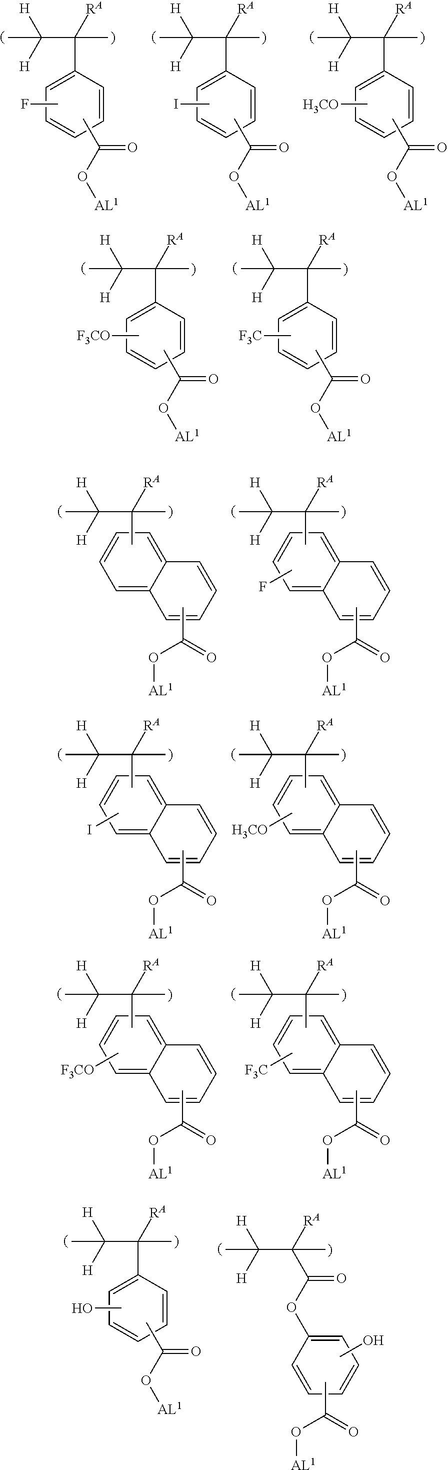

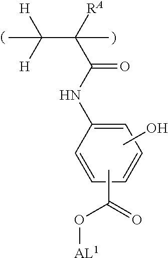

Examples of repeat unit (a2) are shown below, but not limited thereto. Herein R^{A} and AL² are as defined above.

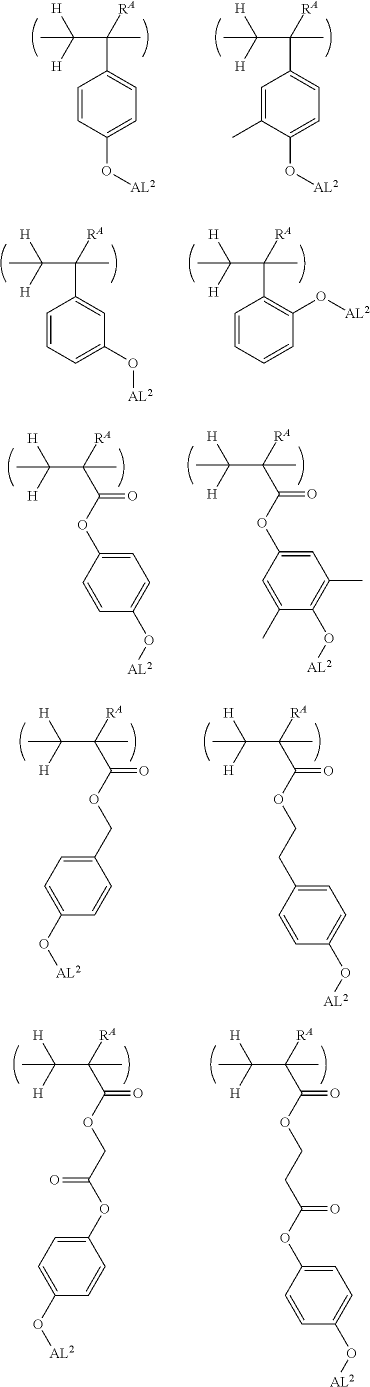

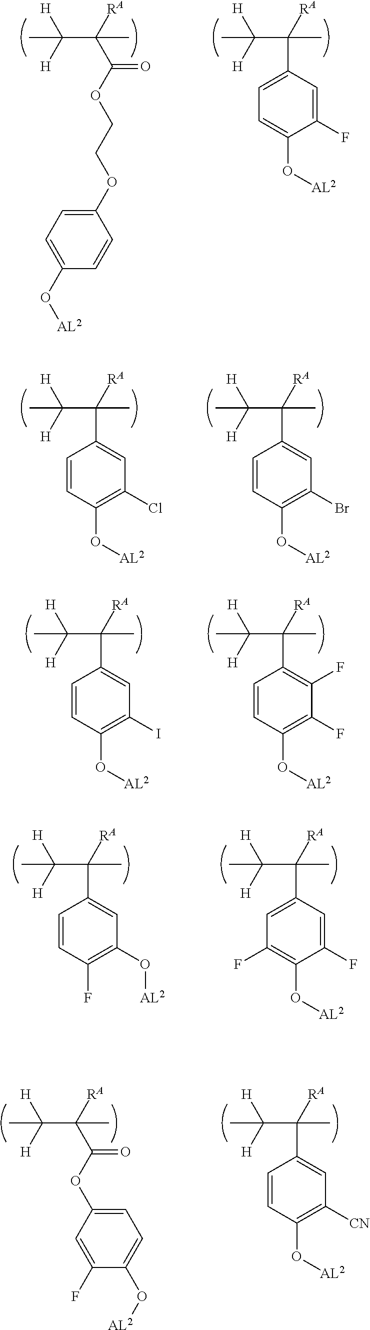

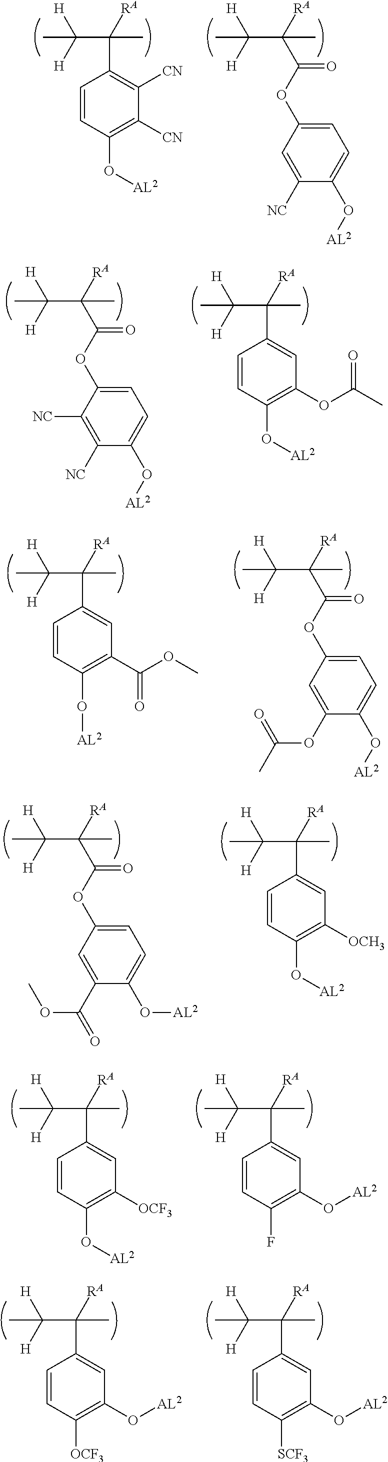

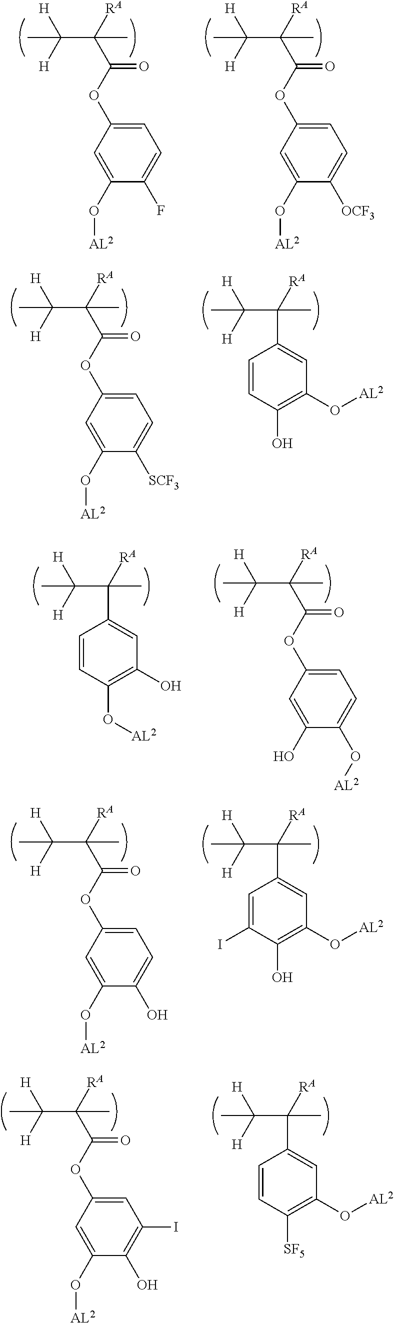

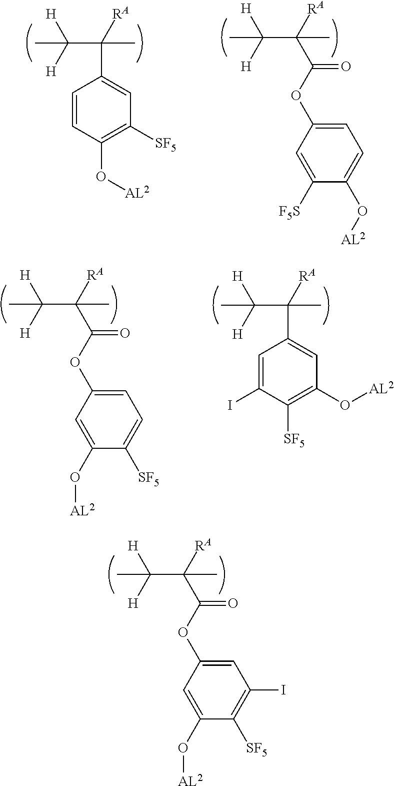

Other examples of the repeat unit having an acid labile group are repeat units having the formula (a3), which are also referred to as repeat units (a3).

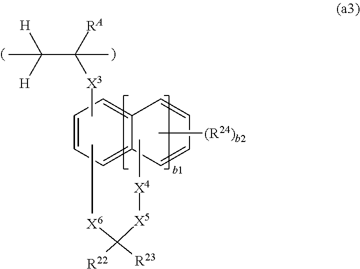

In formula (a3), b1 is 0 or 1. The relevant structure is a benzene ring when b1=0, and a naphthalene ring when b1=1. The benzene ring corresponding to b1=0 is preferred from the aspect of solvent solubility. The subscript b2 is 0, 1, 2 or 3 when b1-0 and b2 is 0, 1, 2, 3, 4 or 5 when b1=1. It is preferred from the aspect of reactant availability that b2 be 0, 1, 2 or 3, more preferably 0, 1 or 2.

In formula (a3), R^{A} is hydrogen, fluorine, methyl or trifluoromethyl. R^{A} is preferably hydrogen or methyl, more preferably hydrogen.

In formula (a3), X³ is a single bond, *—C(═O)—O— or *—C(═O)—N(H)— wherein * designates a point of attachment to the carbon atom in the backbone. X³ is preferably a single bond or *—C(—O)—O—, more preferably a single bond.

In formula (a3), X⁴ is a single bond, C₁-C₄ aliphatic hydrocarbylene group, carbonyl group, sulfonyl group or a group obtained by combining the foregoing. It is preferred from the aspect of reactant availability that X⁴ be a single bond, carbonyl or sulfonyl. It is more preferred from the aspect of a polar group created after reaction that X⁴ be a single bond or carbonyl.

In formula (a3), X⁵ and X⁶ are each independently oxygen or sulfur, with the proviso that X⁴ and X⁶ are attached to adjoining carbon atoms on the aromatic ring. X⁵ and X⁶ may be the same or different. It is preferred from the aspect of reactivity that X⁵ and X⁶ be both oxygen.

In formula (a3), R²² and R²³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(—O)—) or haloalkyl moiety.

Also, R²² and R²³ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In formula (a3), R²⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}). R^{24A} and R^{24B} are each independently hydrogen or a C₁-C₆ hydrocarbyl group. Suitable halogen atoms include fluorine, chlorine, bromine and iodine, with fluorine and iodine being preferred. The hydrocarbyl group and hydrocarbyl moiety in the hydrocarbyloxy, hydrocarbyloxycarbonyl, and hydrocarbylthio groups may be saturated or unsaturated and straight, branched or cyclic. Examples thereof are as exemplified above for the hydrocarbyl groups R²² and R²³. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. When b2 is 2 or more, a plurality of R²⁴ may be identical or different.

When b2 is 2 or more, a plurality of R²⁴ may bond together to form a ring with carbon atoms in the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(—O)—) or haloalkyl moiety.

Examples of repeat unit (a3) are shown below, but not limited thereto. Herein R^{A} is as defined above. The positions of attachment of substituent groups on the aromatic ring are interchangeable.

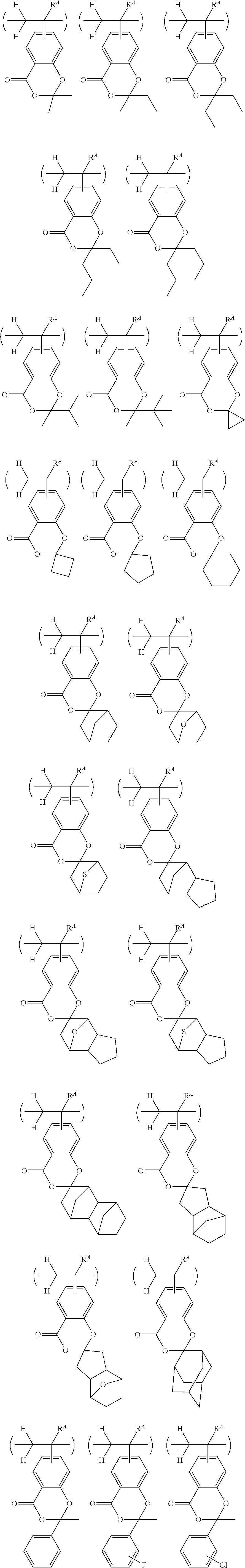

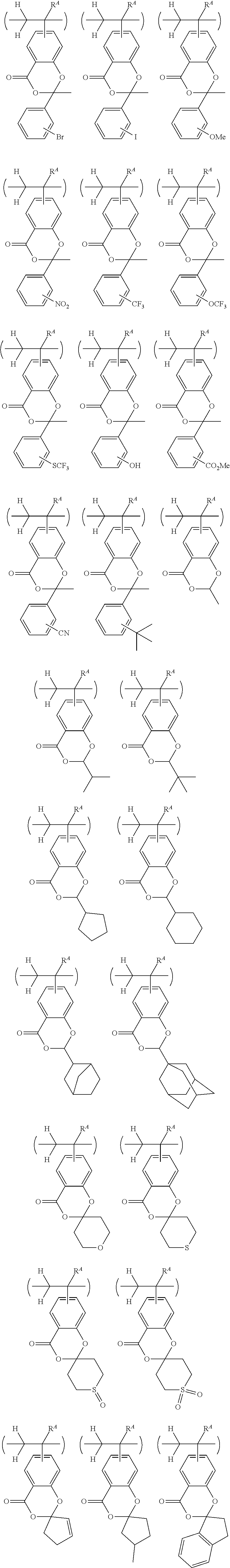

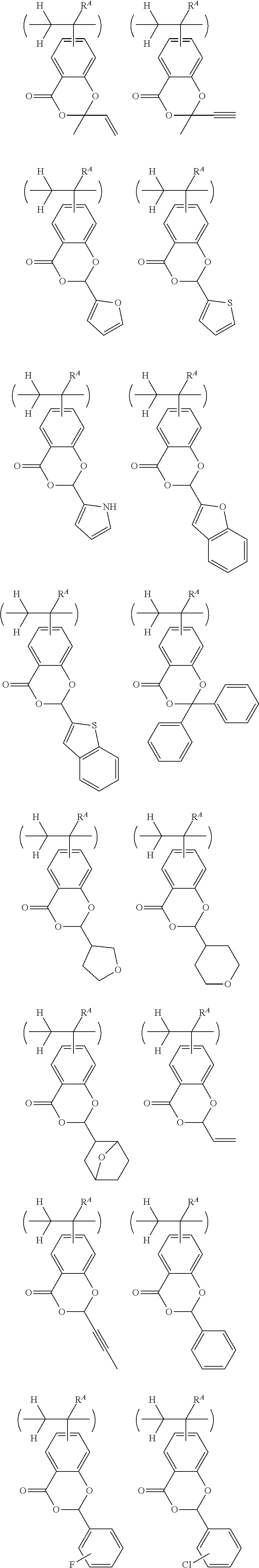

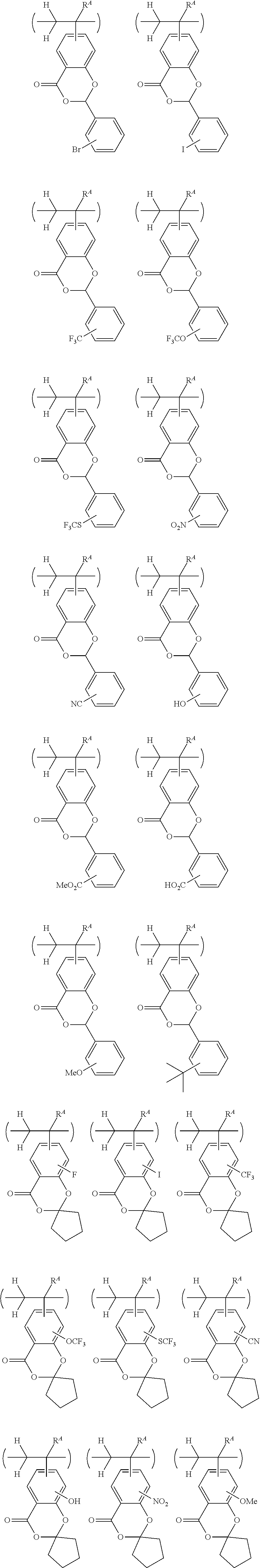

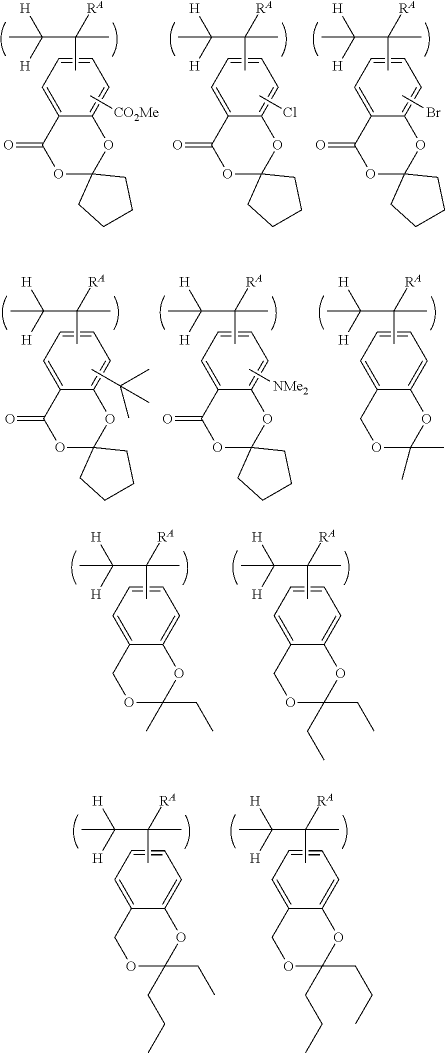

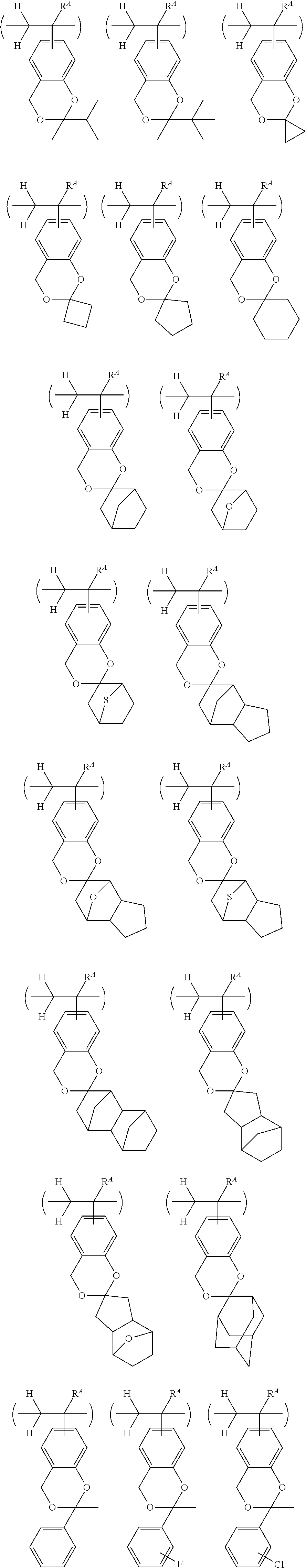

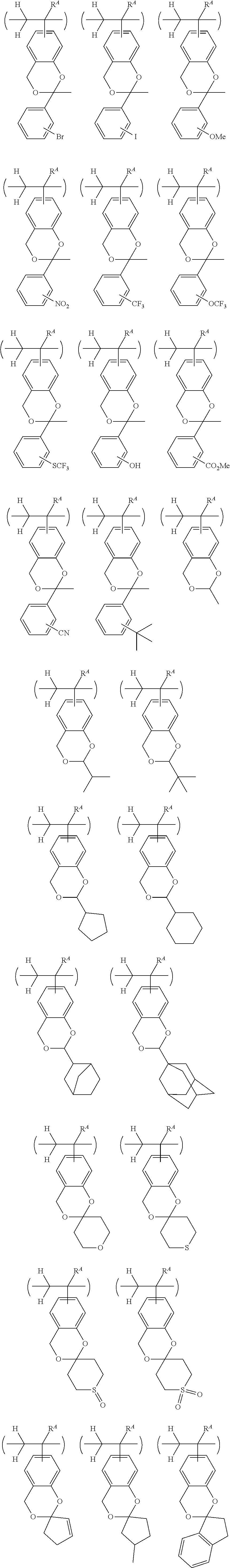

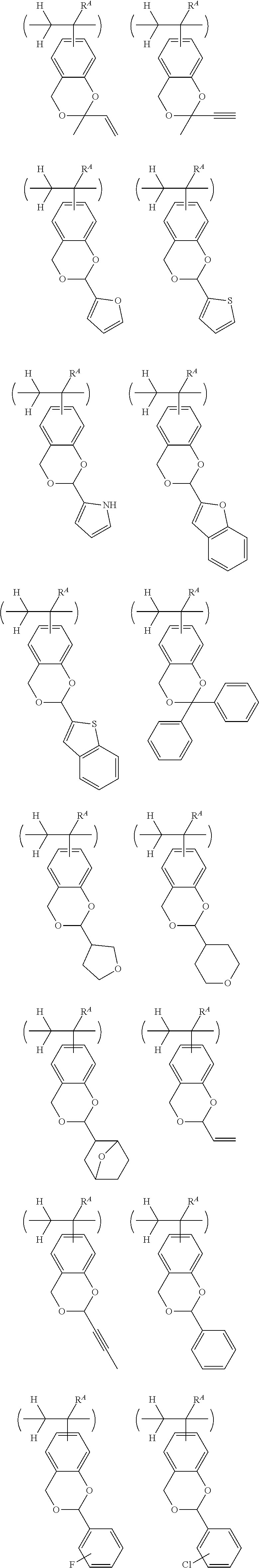

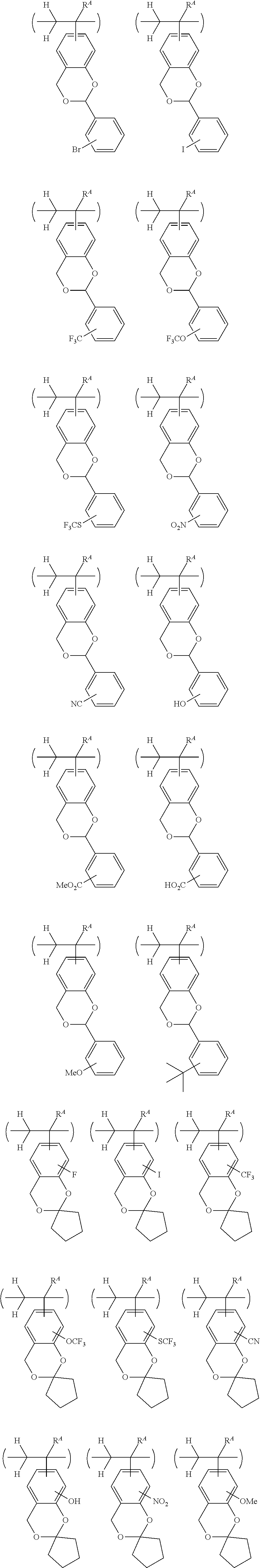

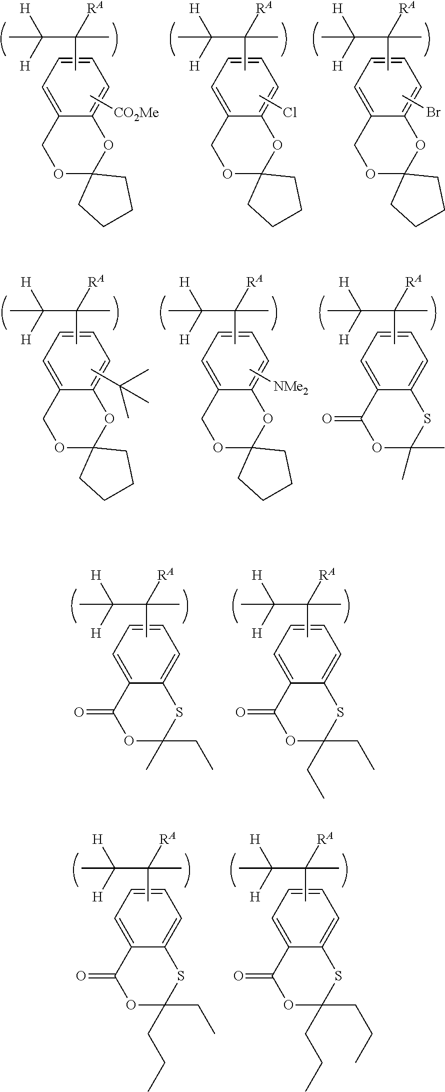

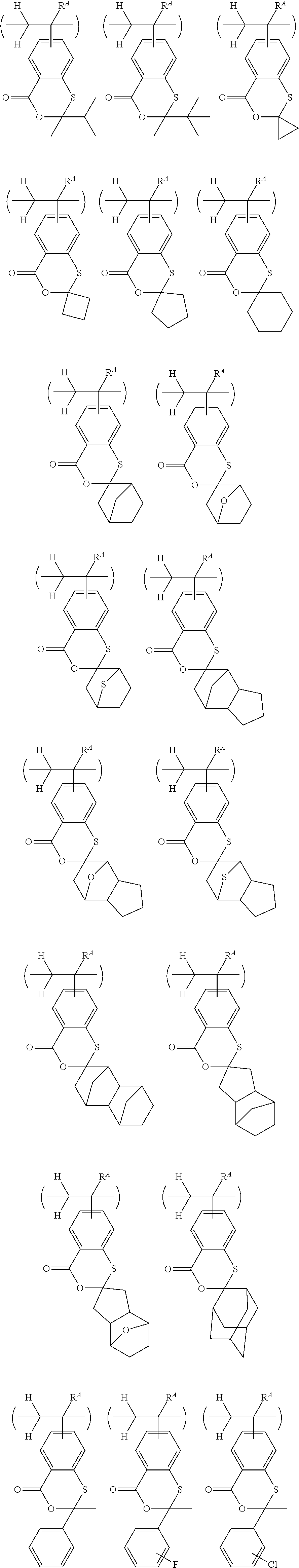

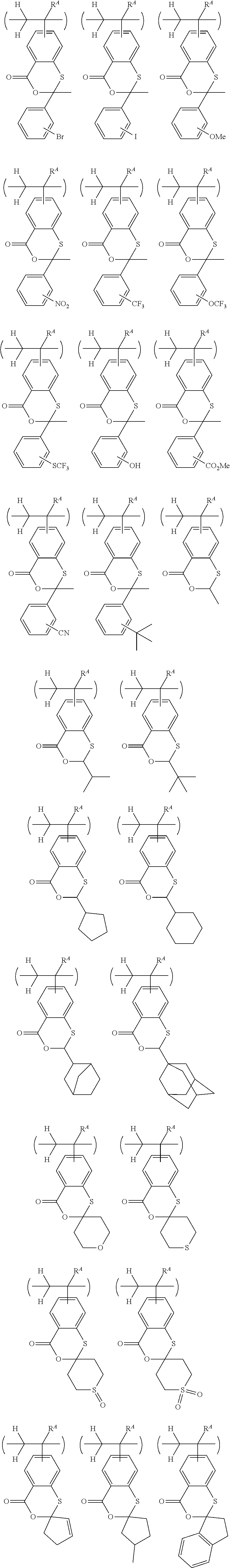

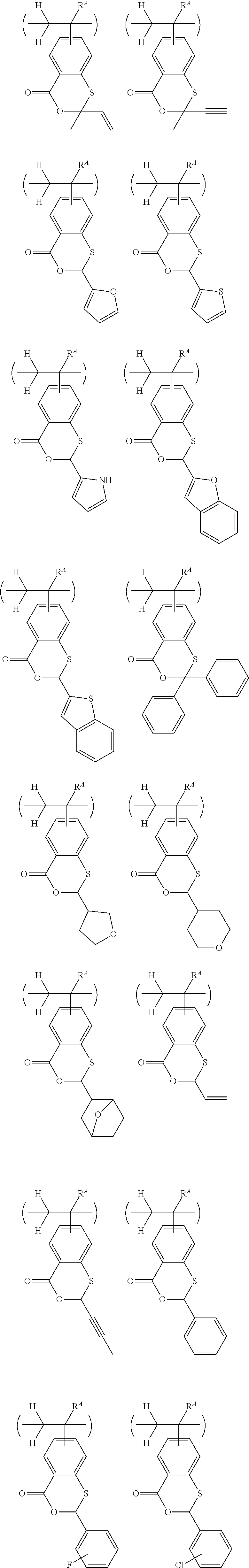

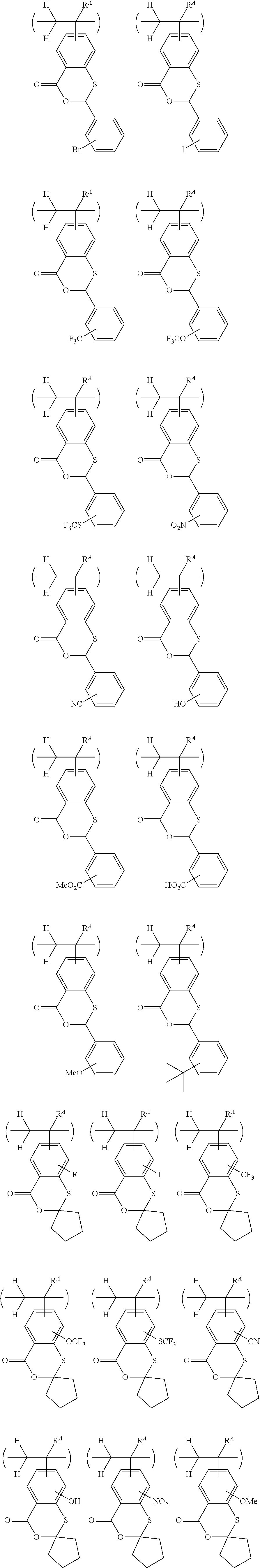

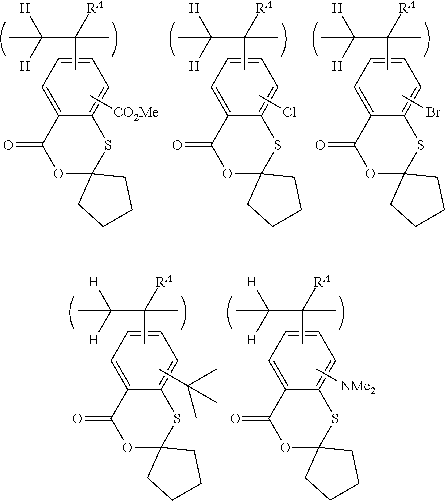

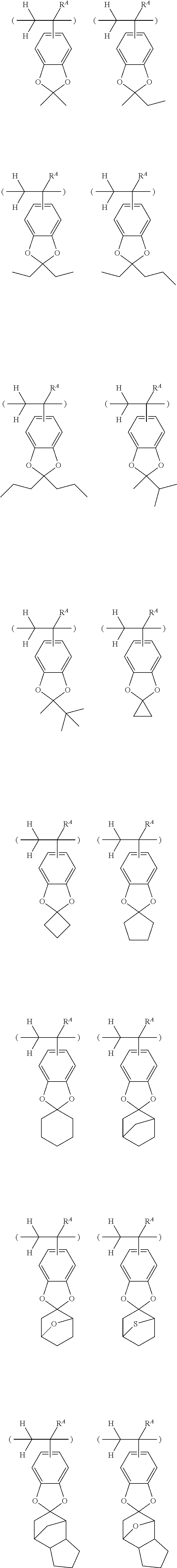

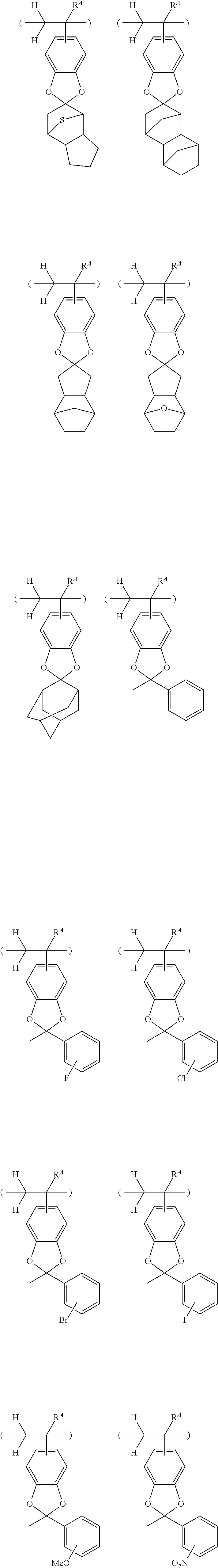

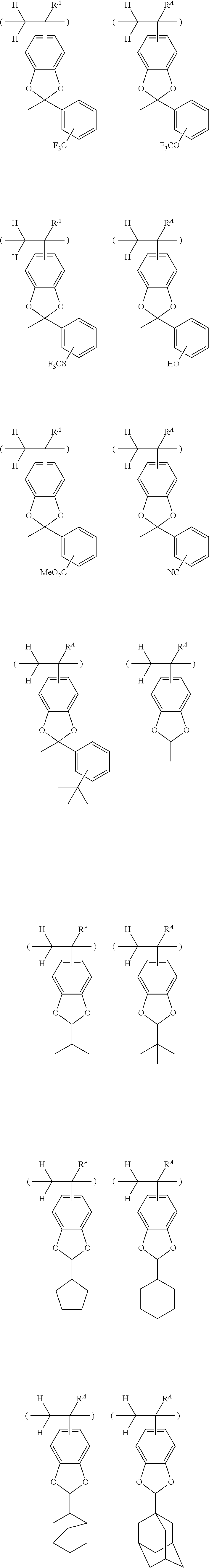

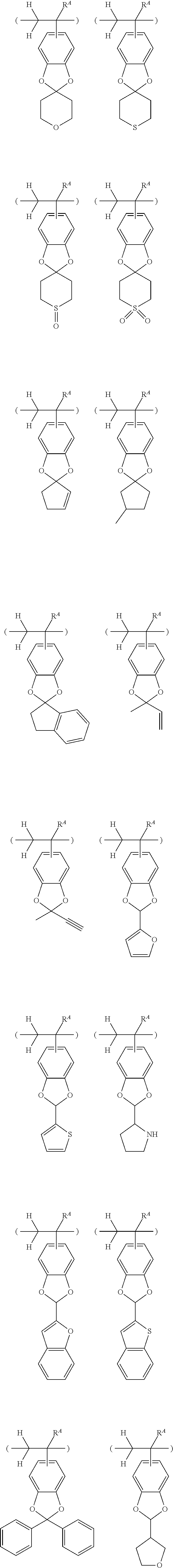

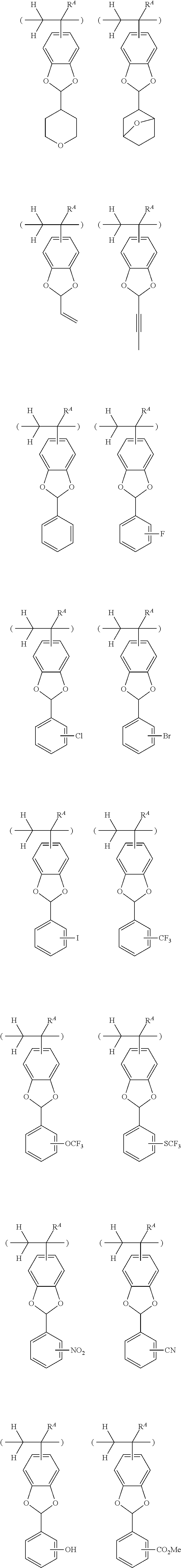

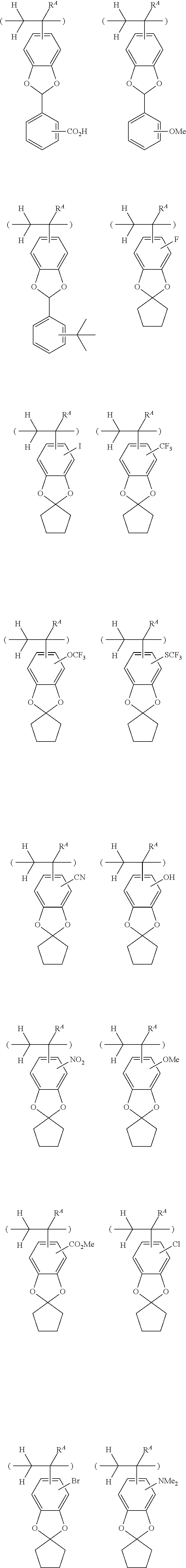

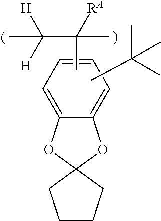

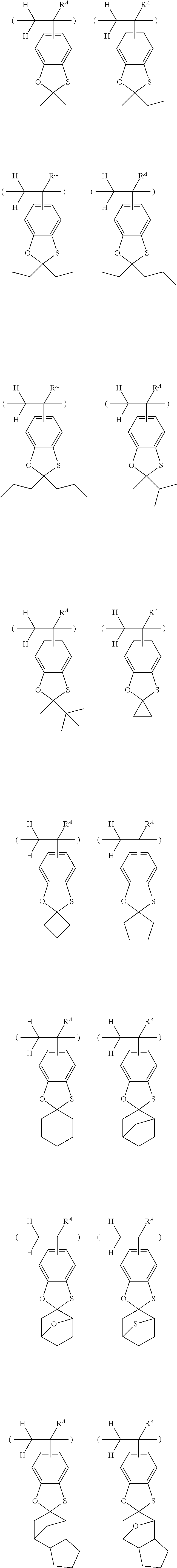

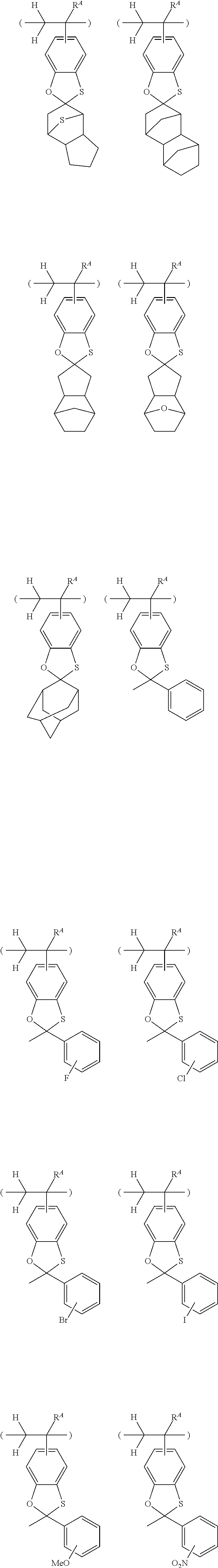

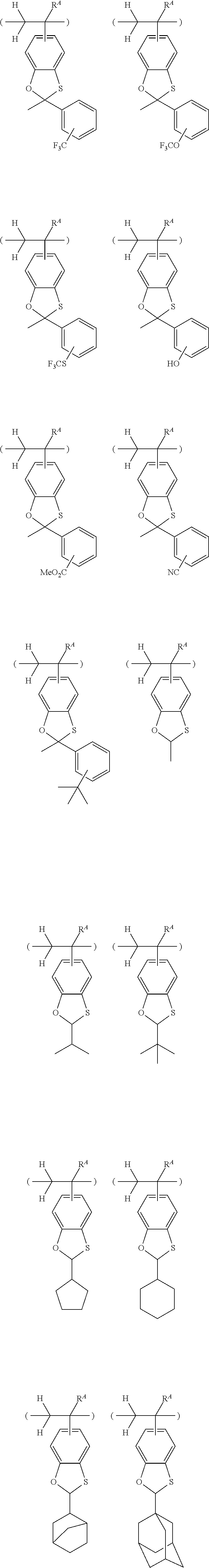

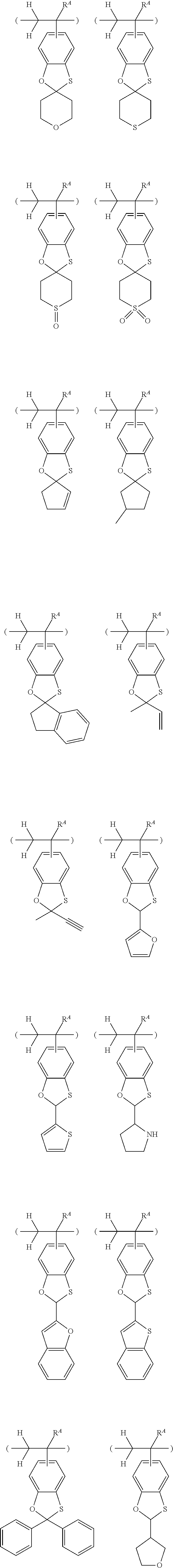

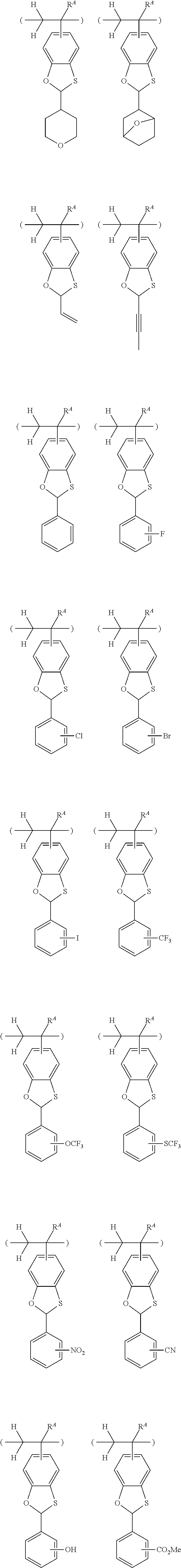

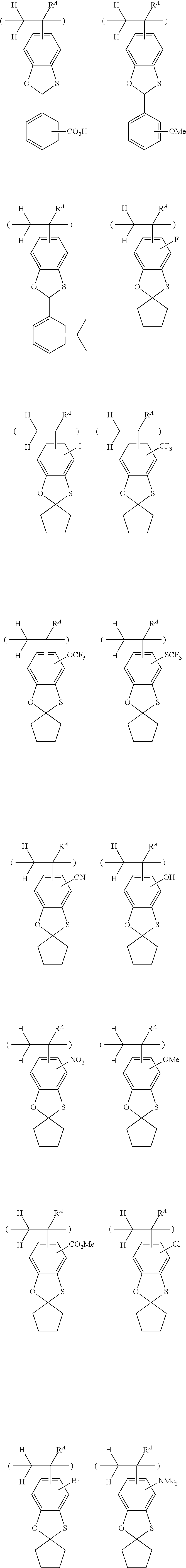

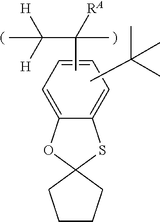

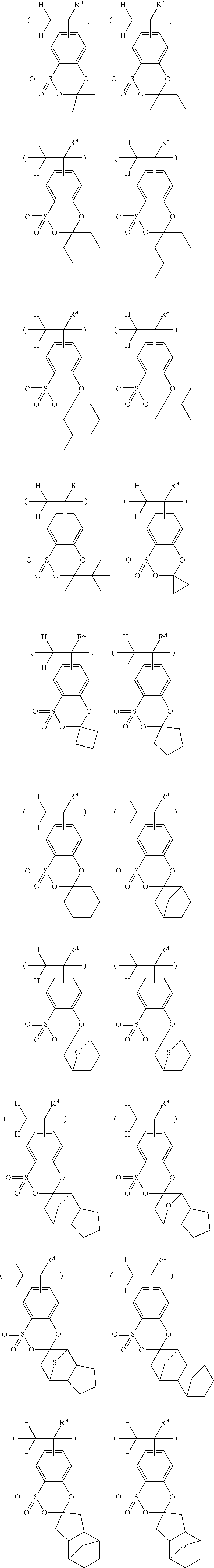

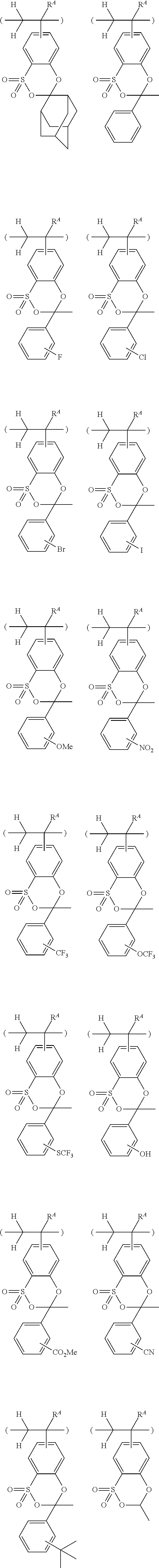

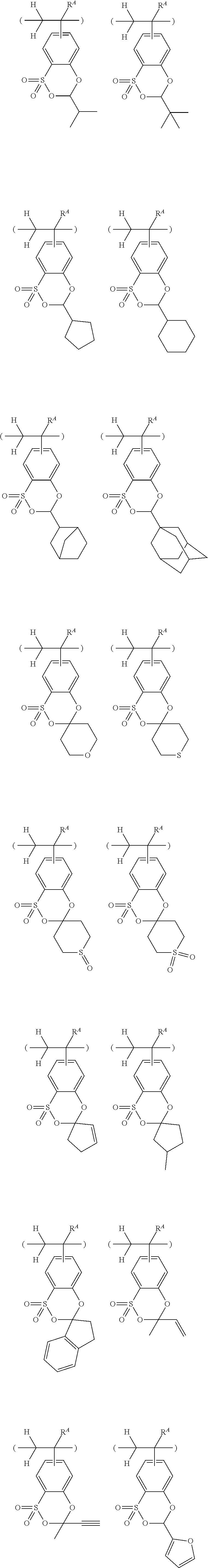

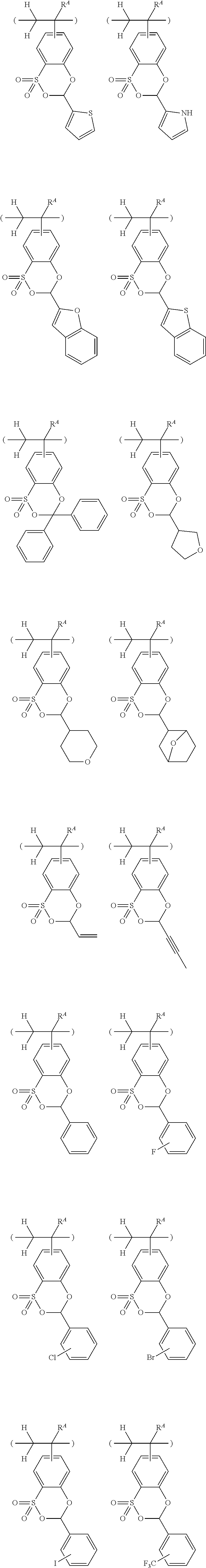

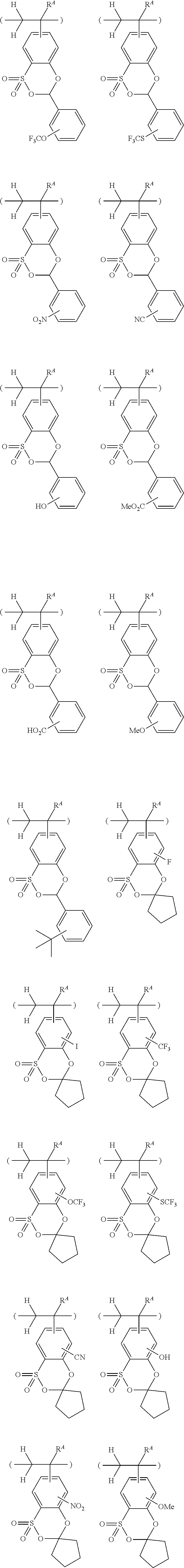

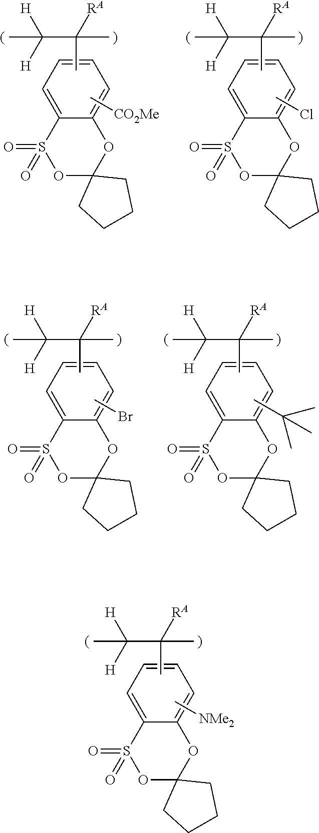

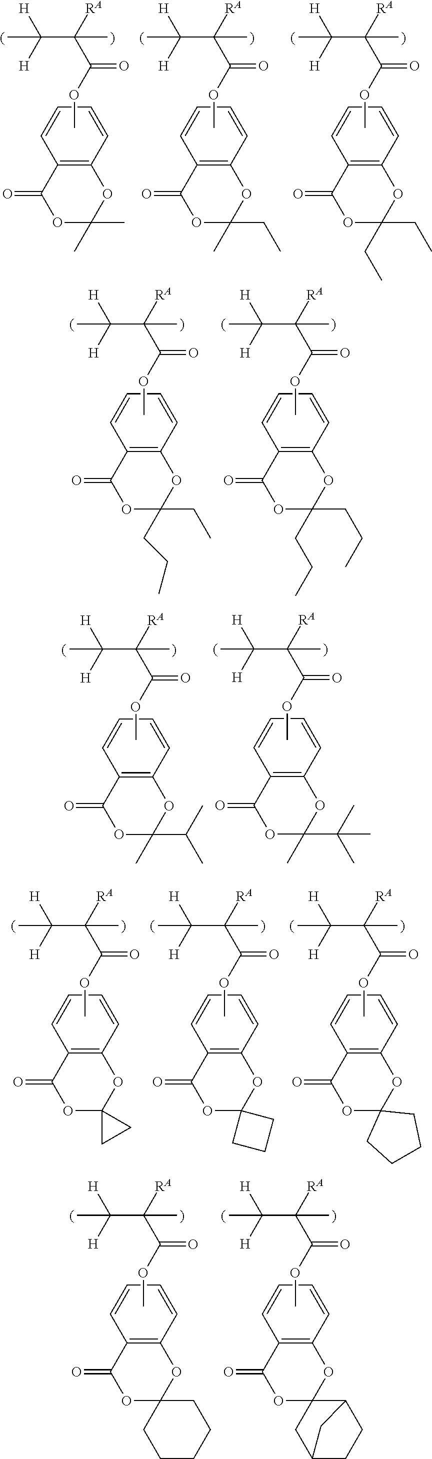

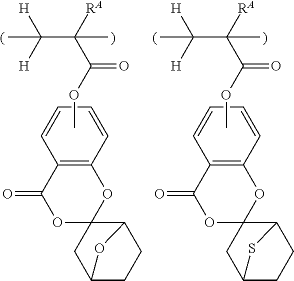

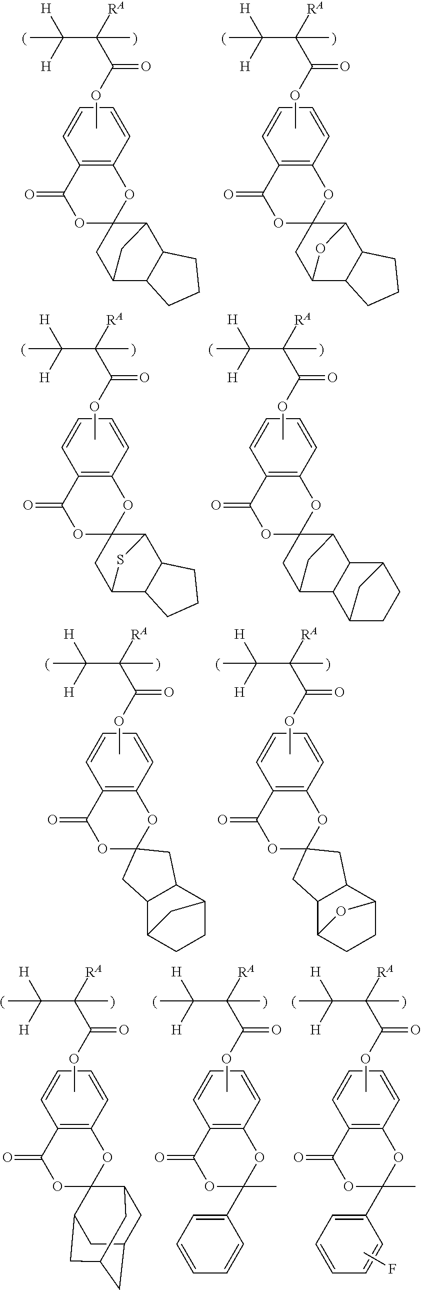

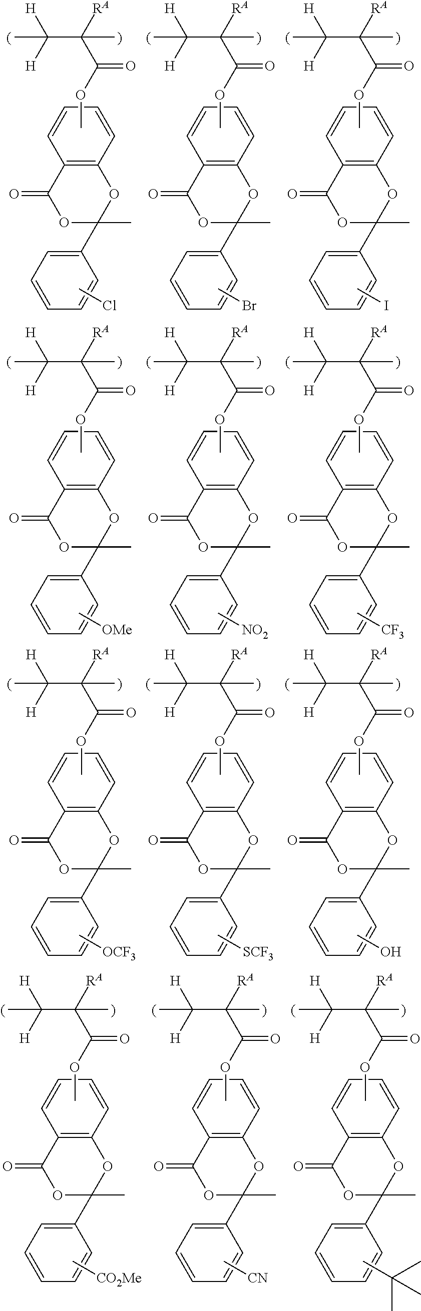

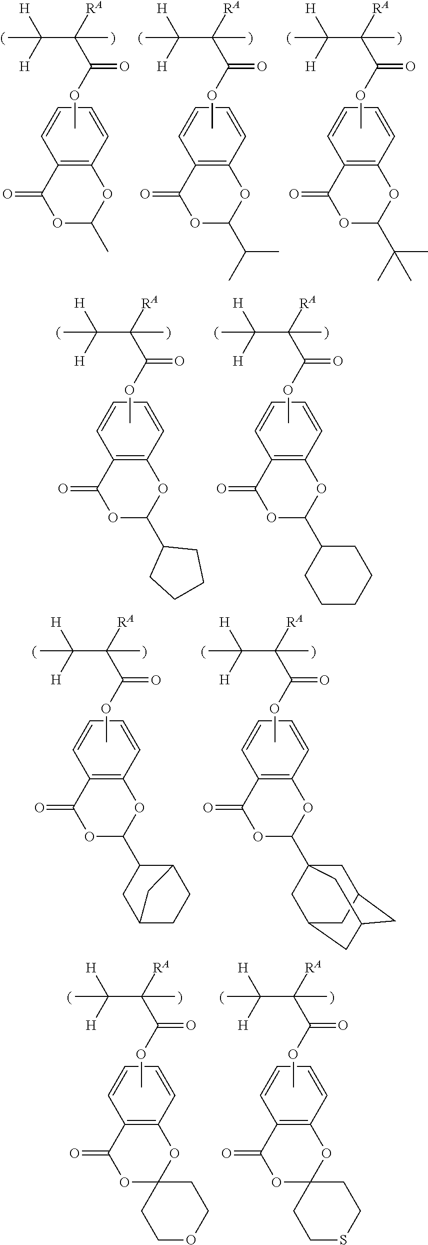

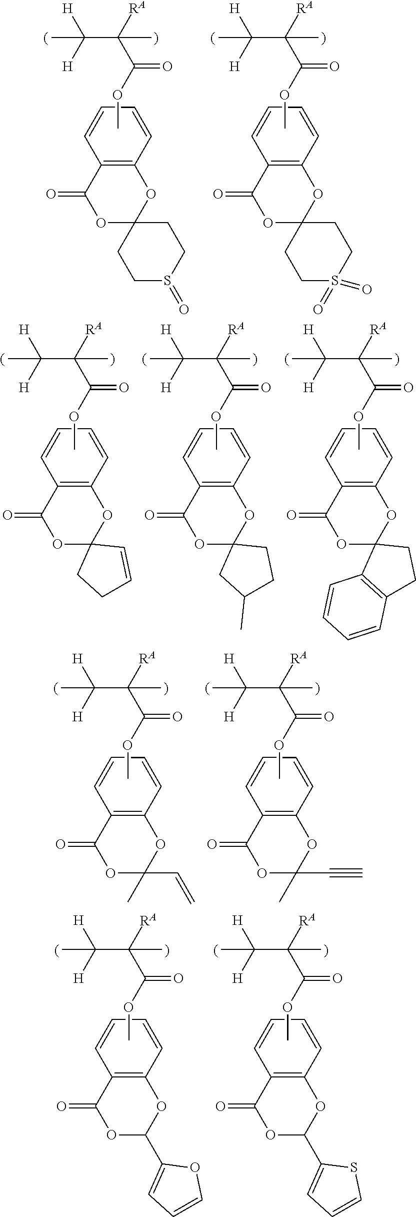

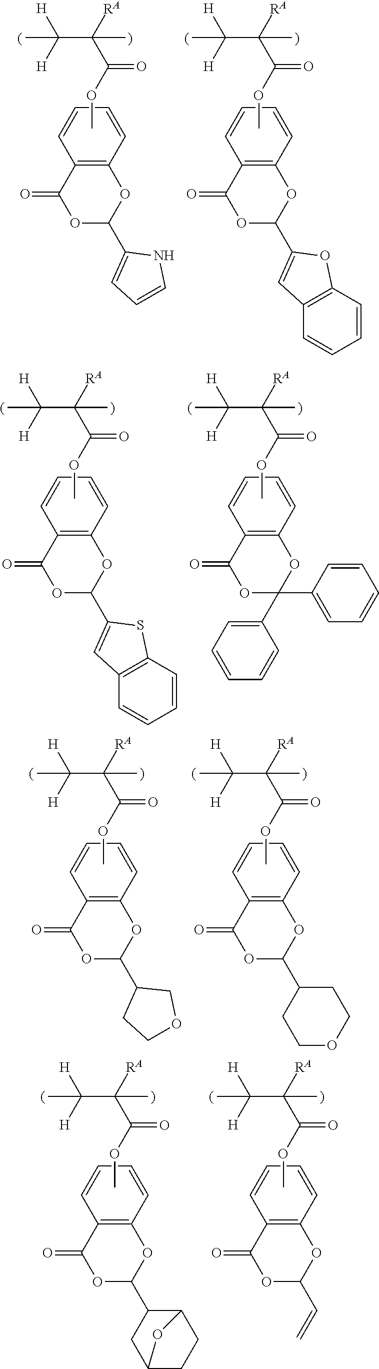

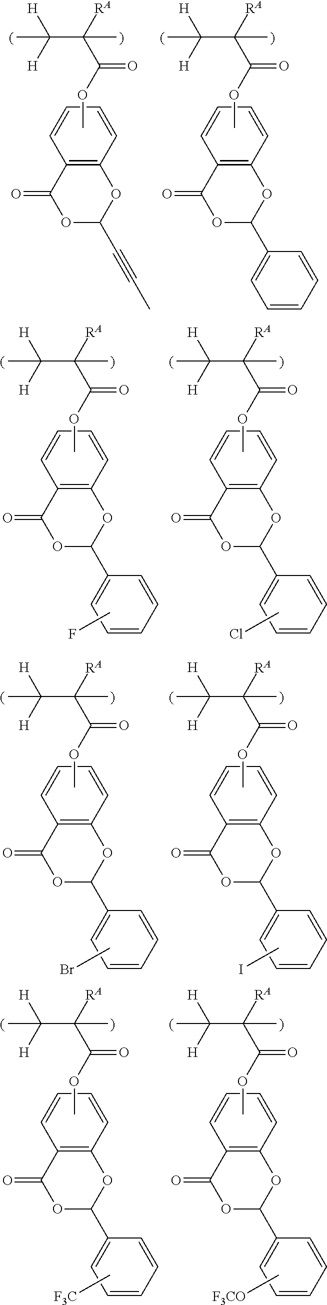

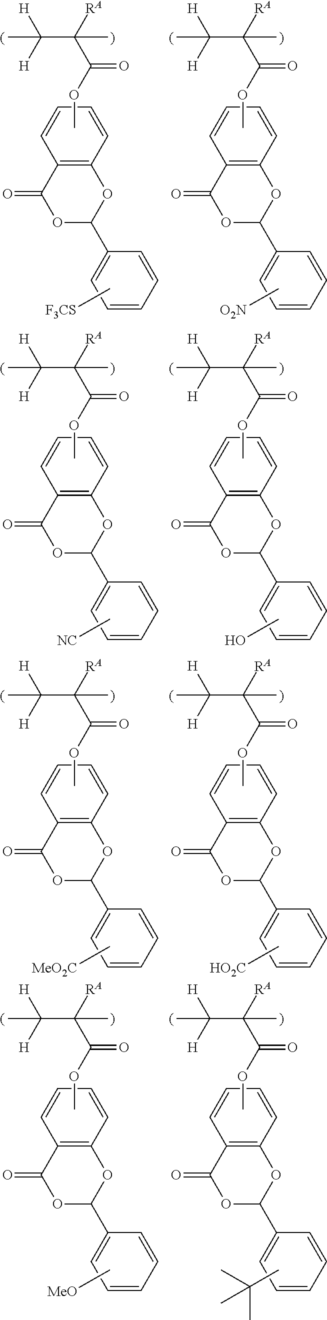

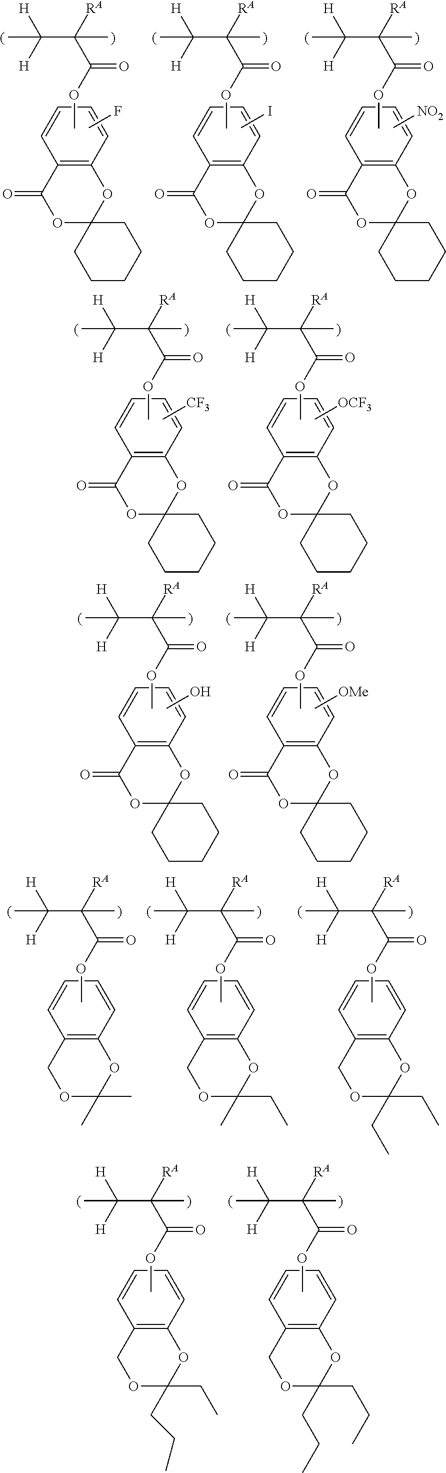

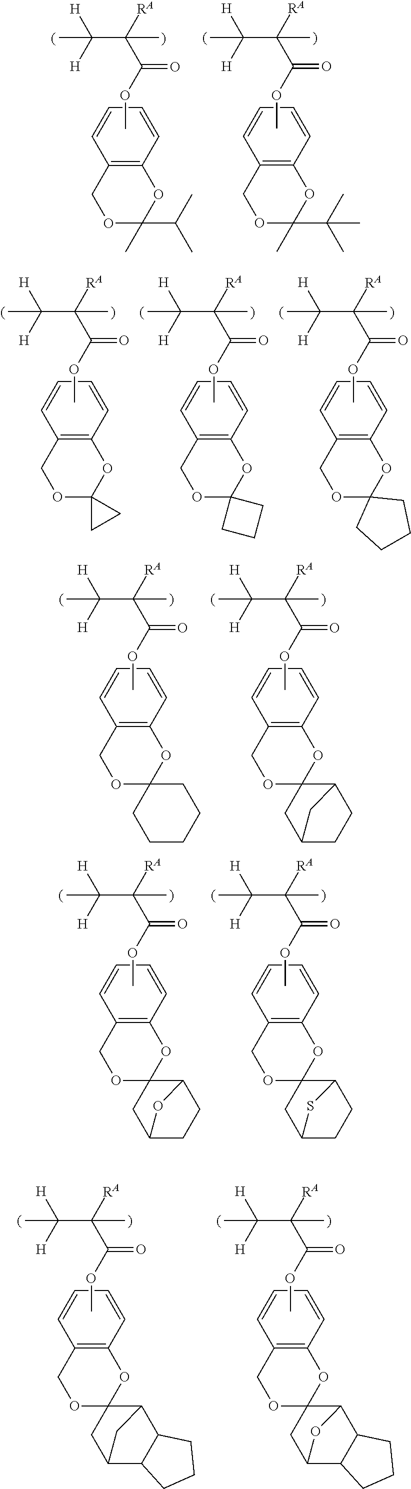

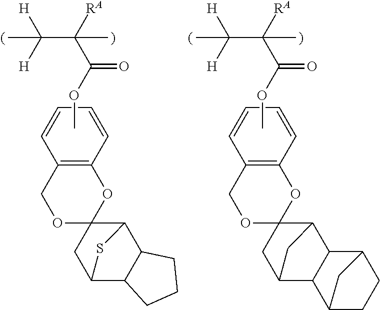

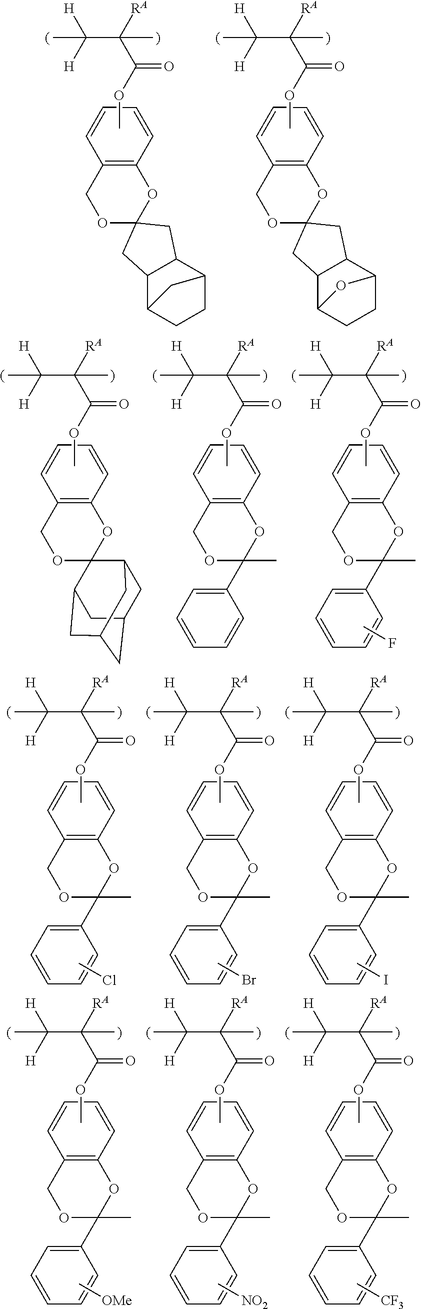

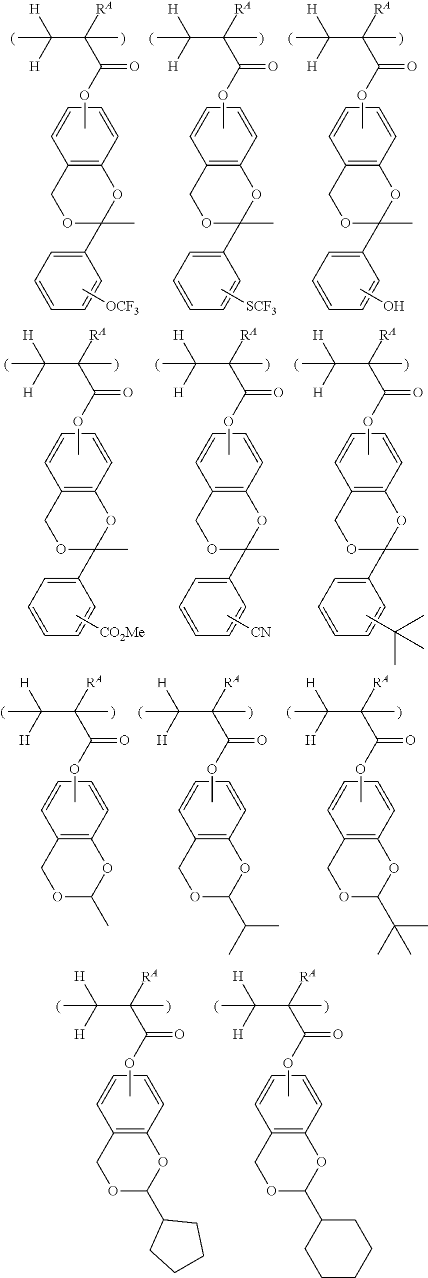

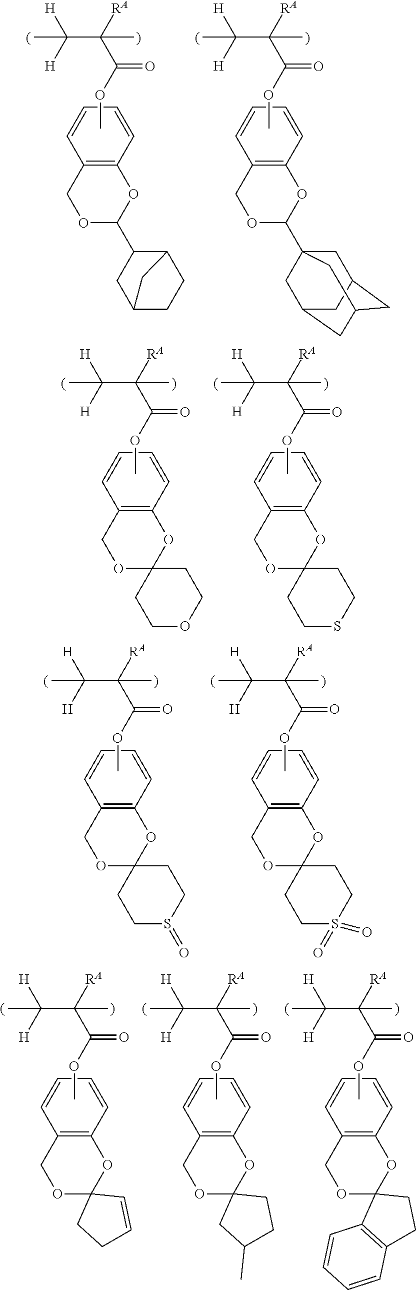

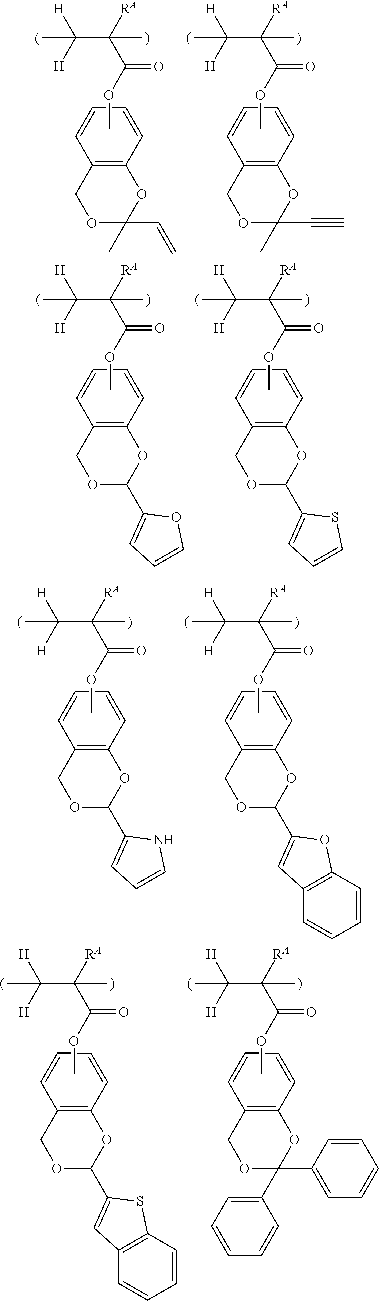

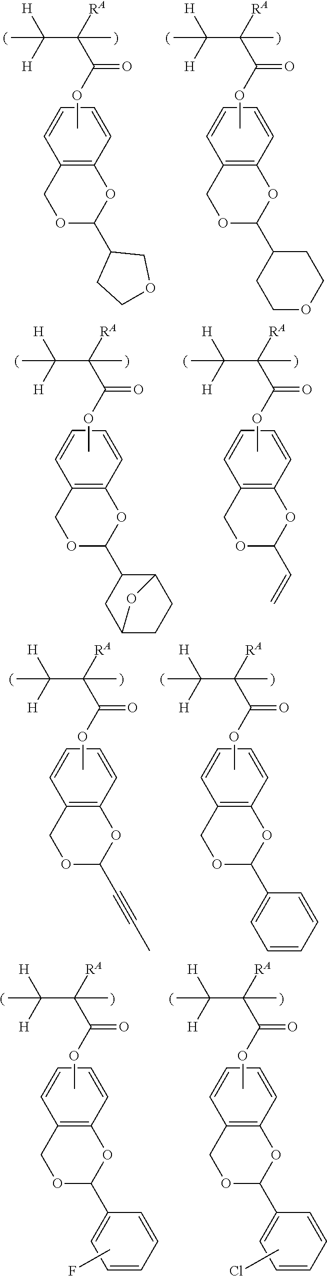

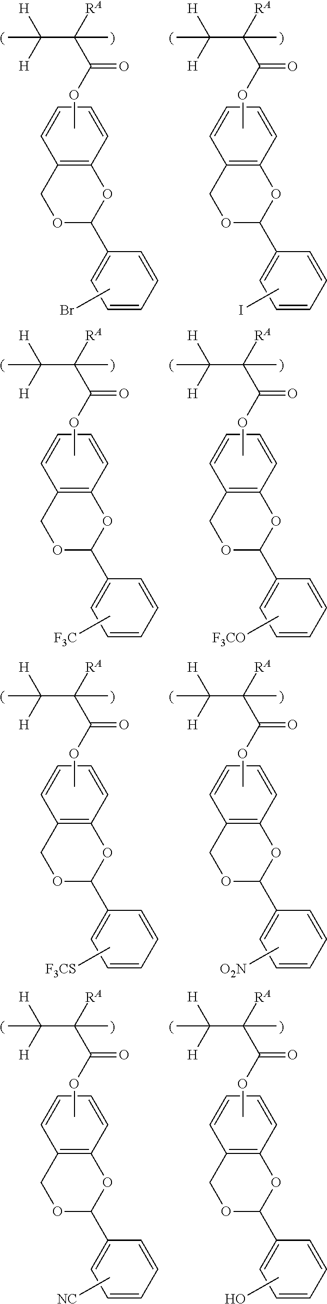

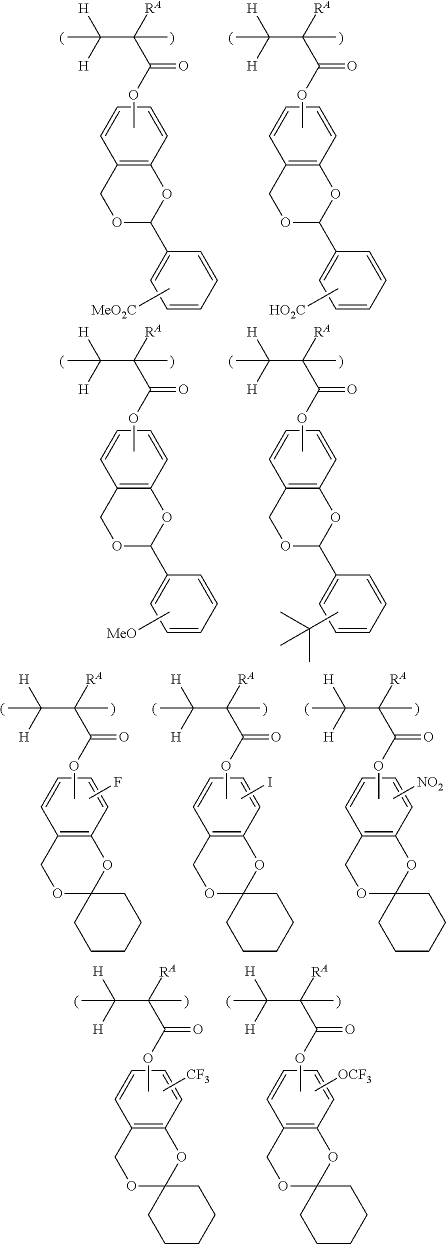

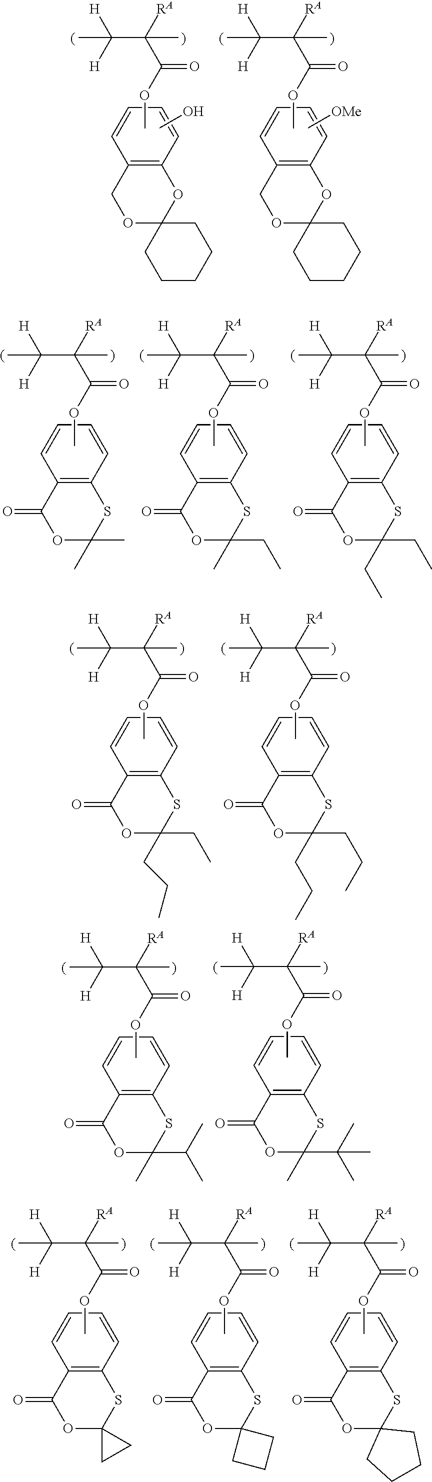

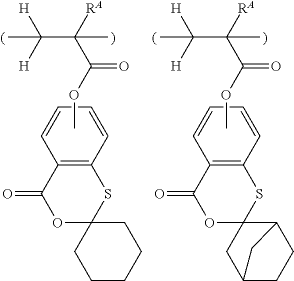

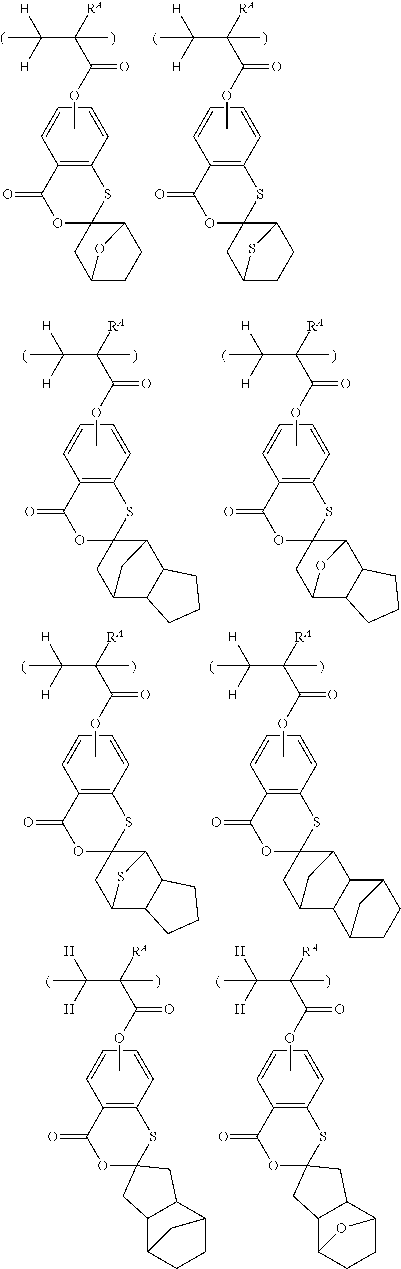

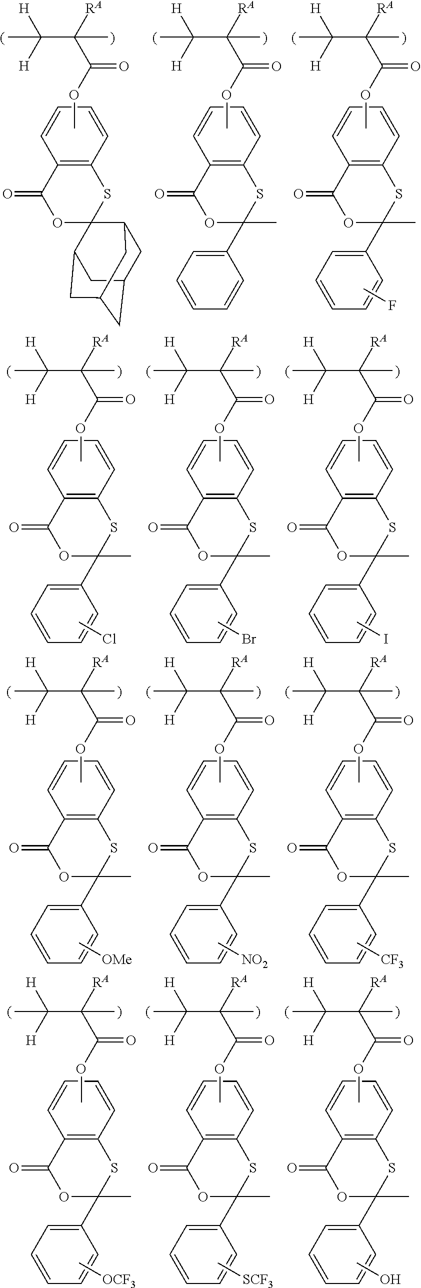

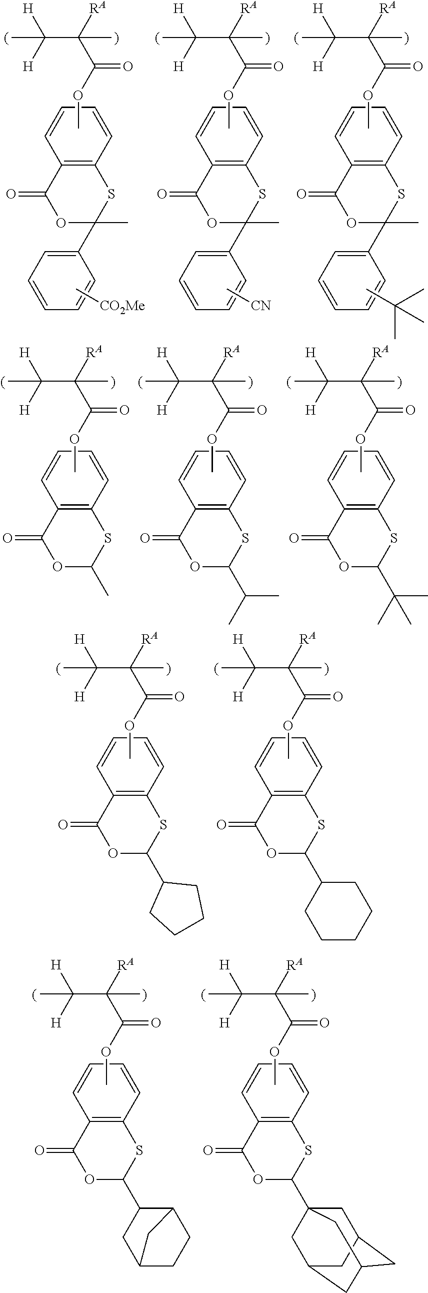

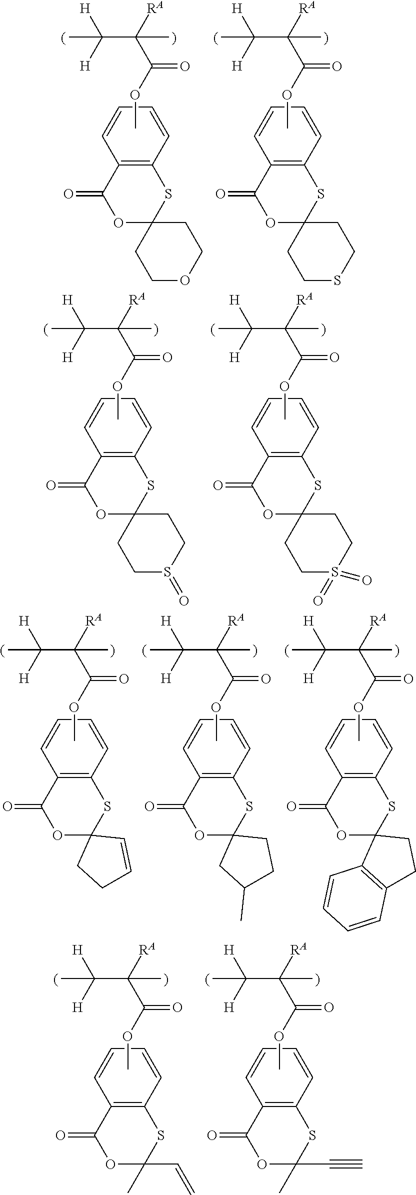

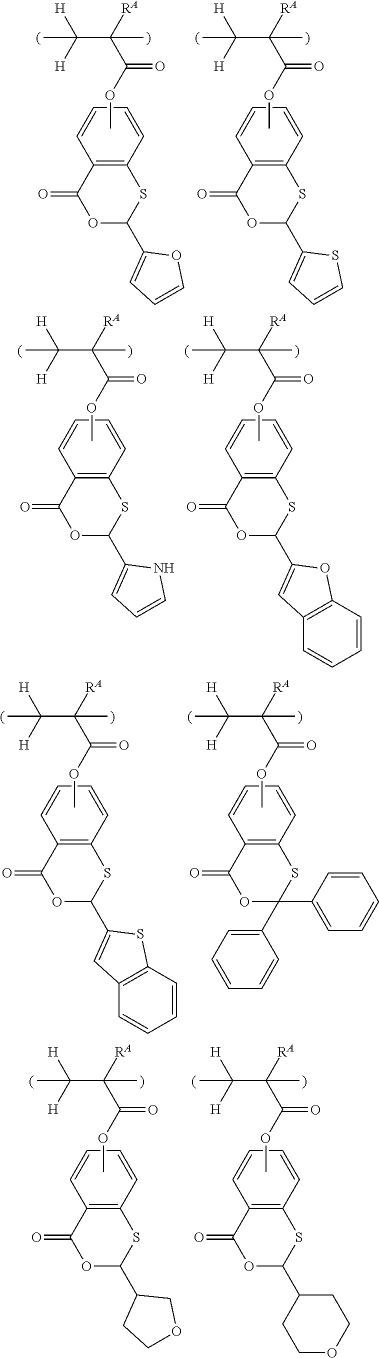

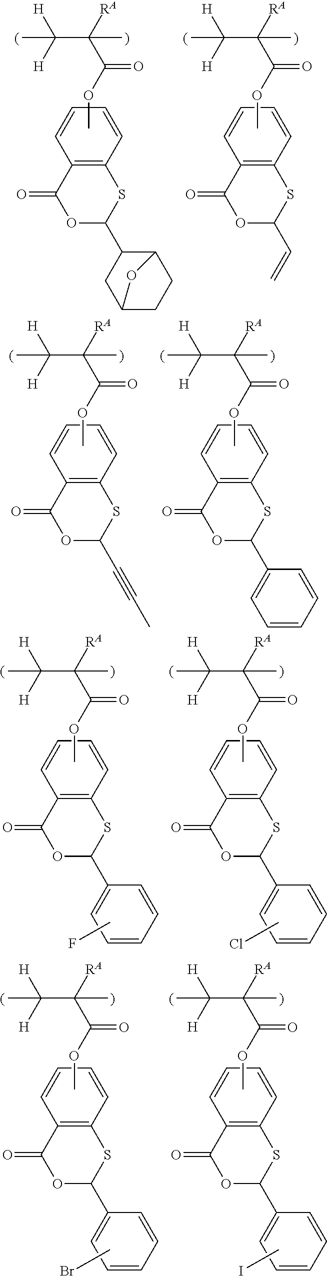

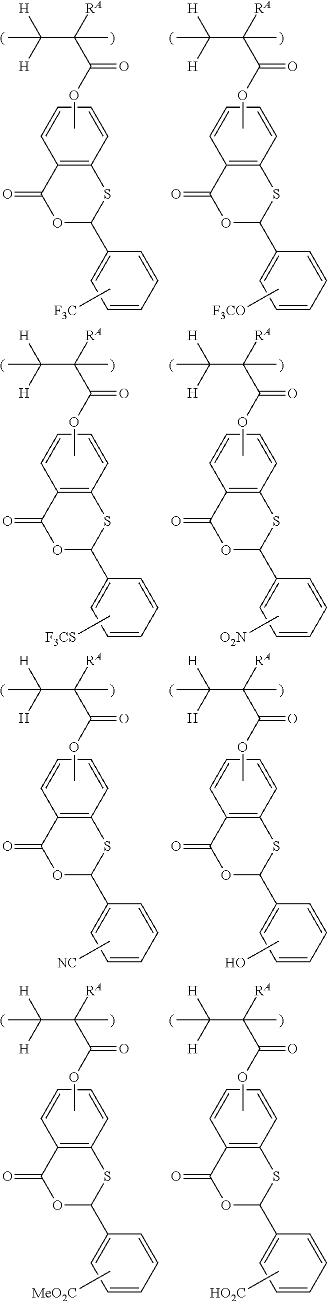

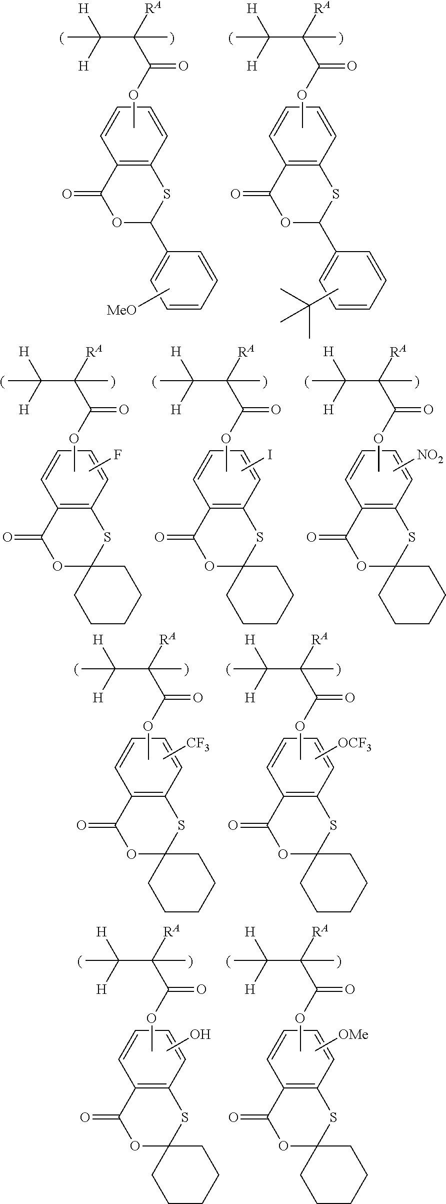

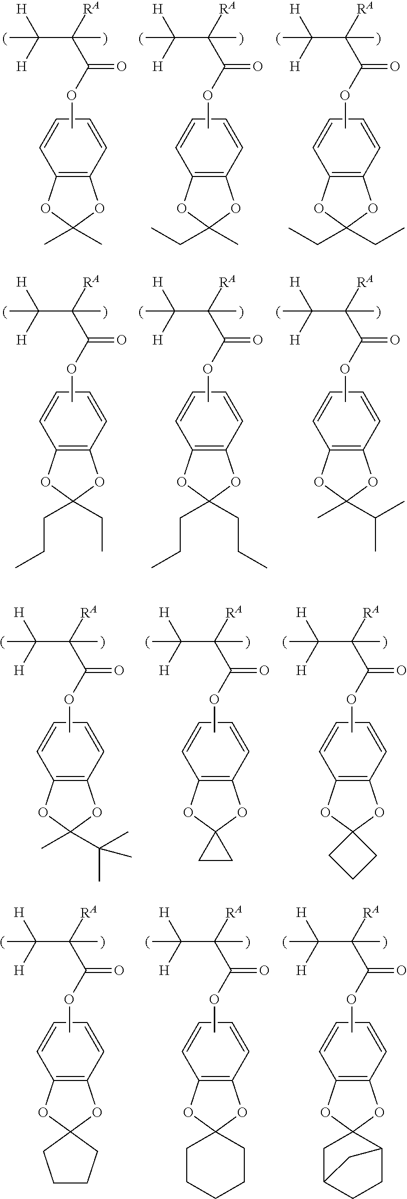

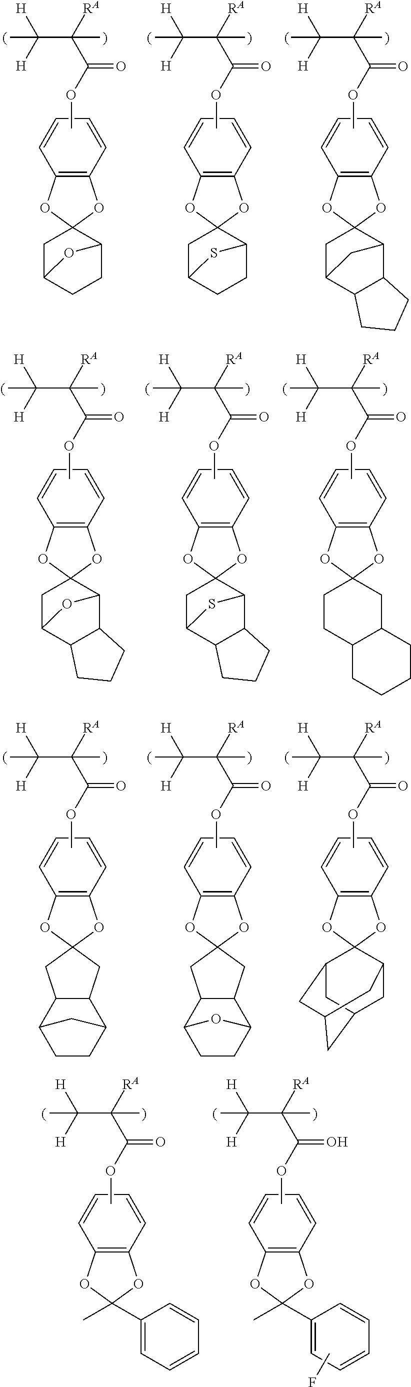

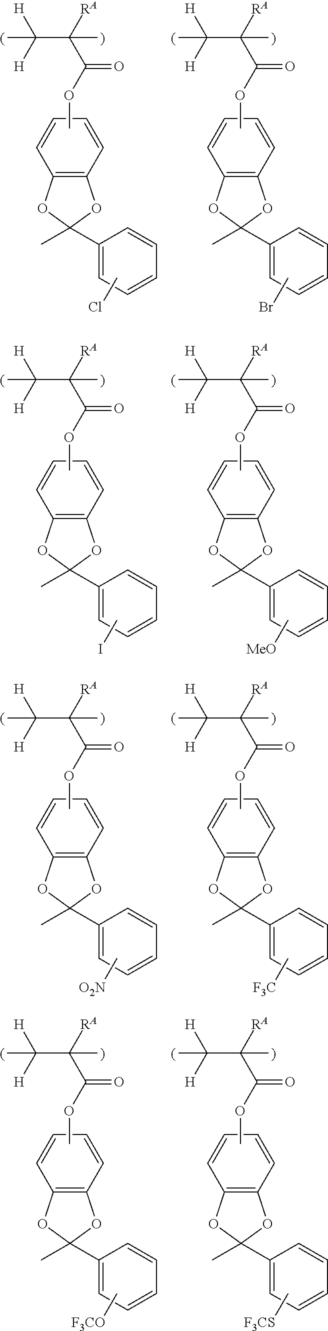

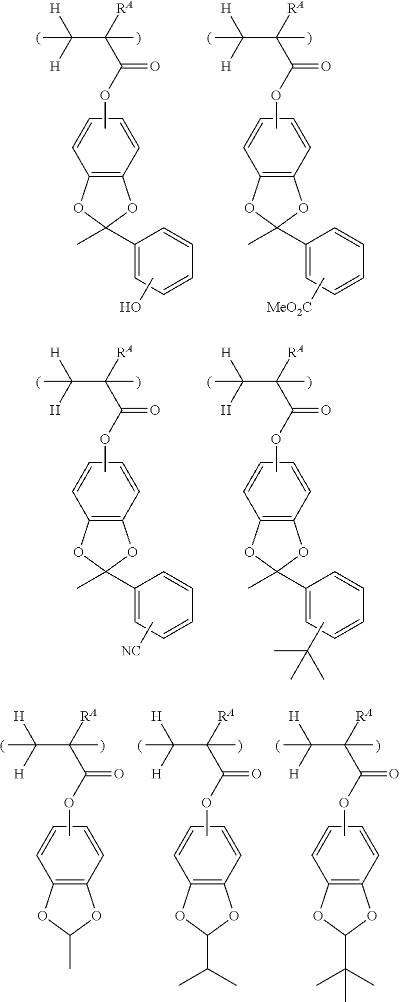

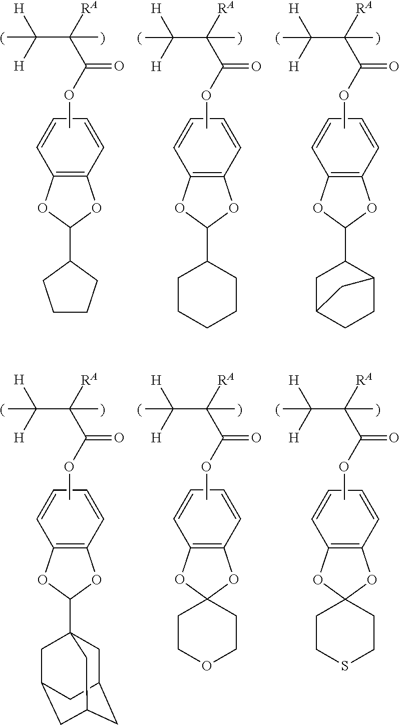

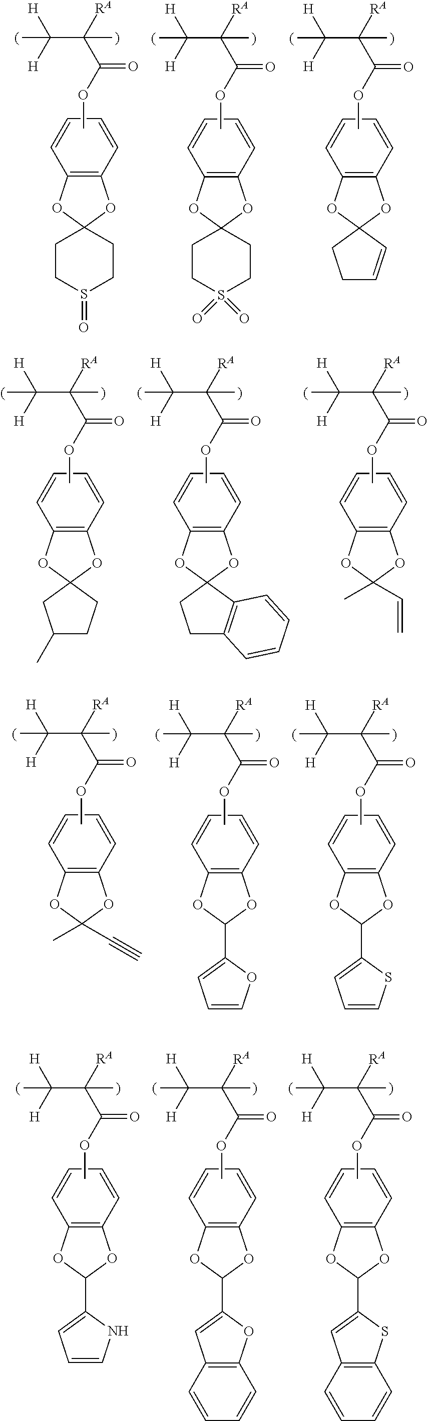

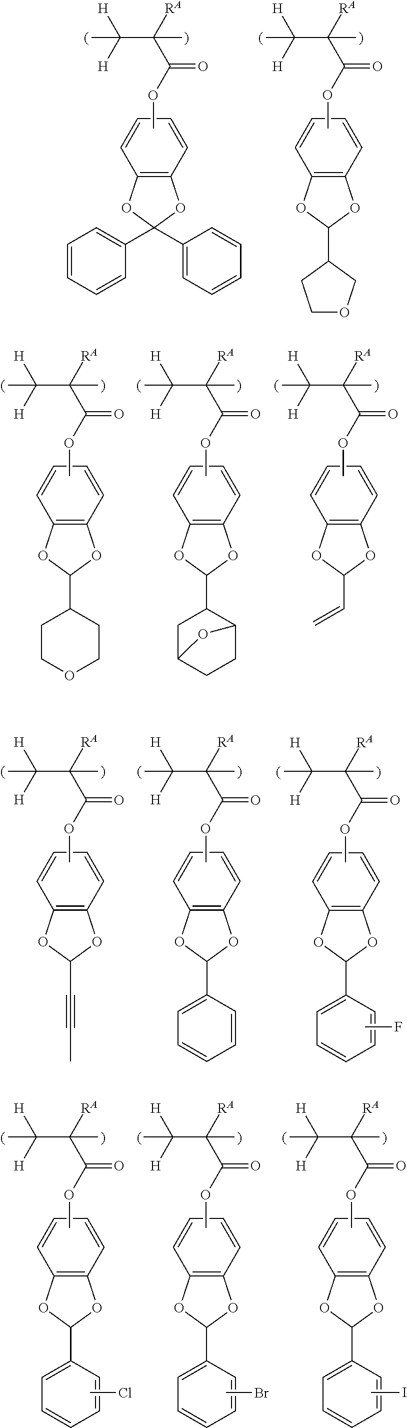

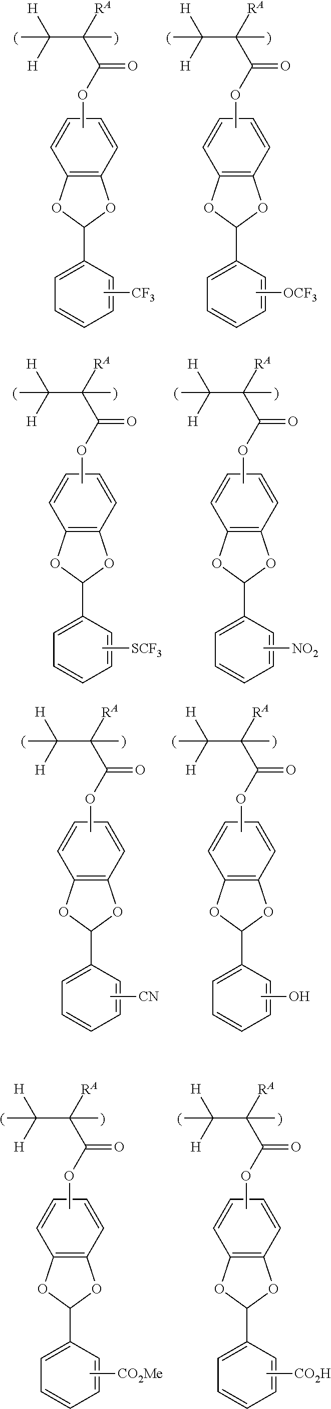

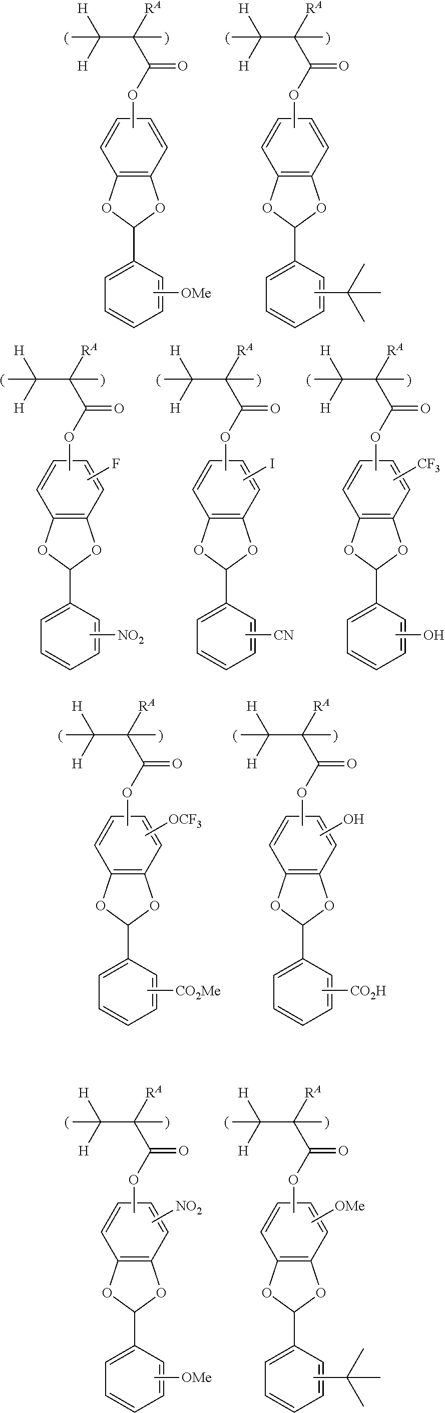

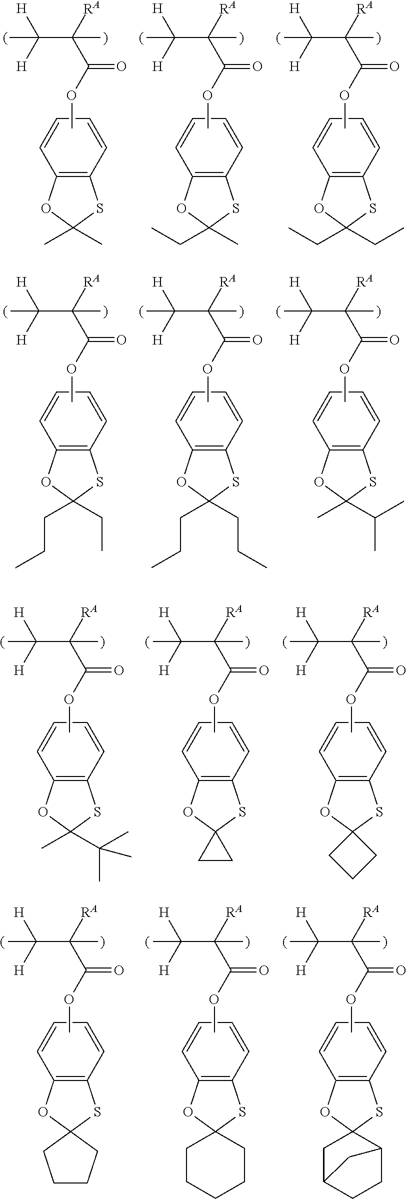

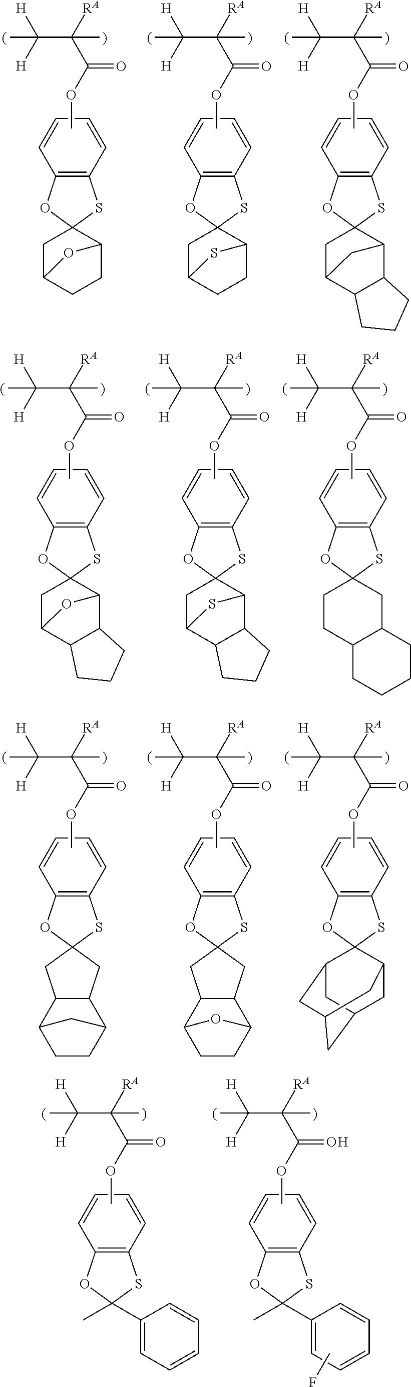

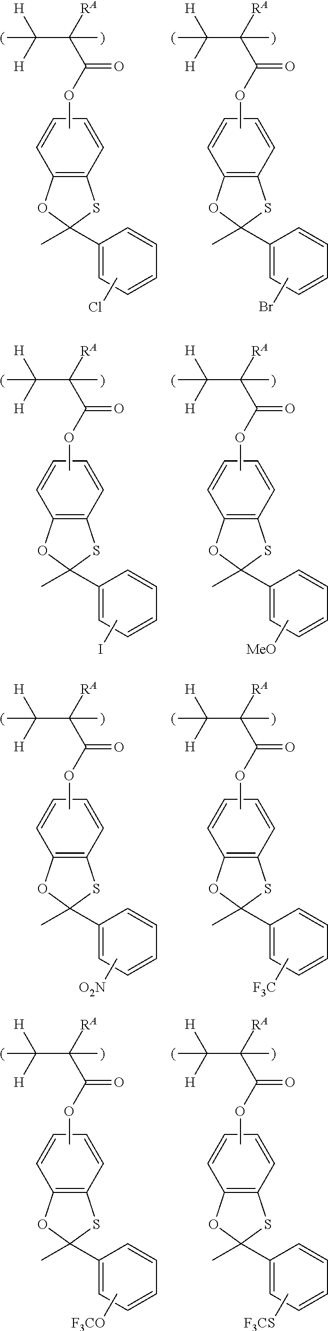

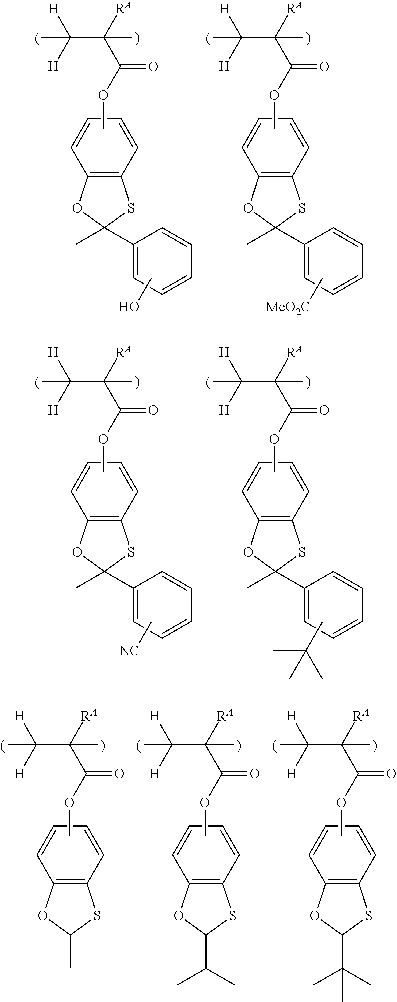

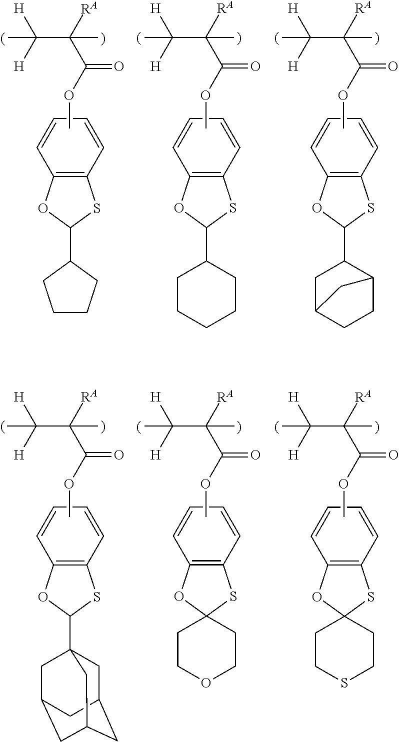

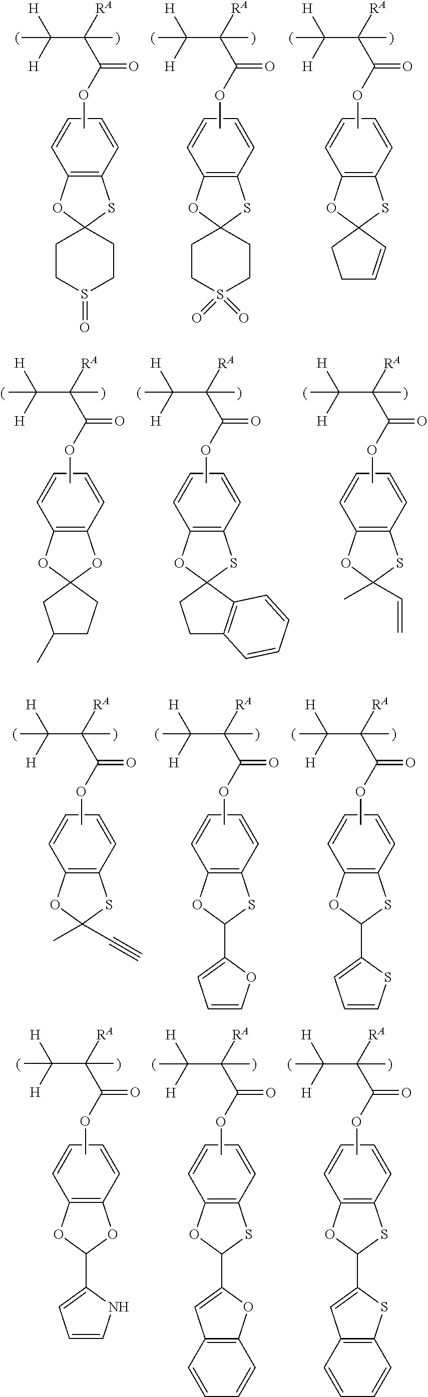

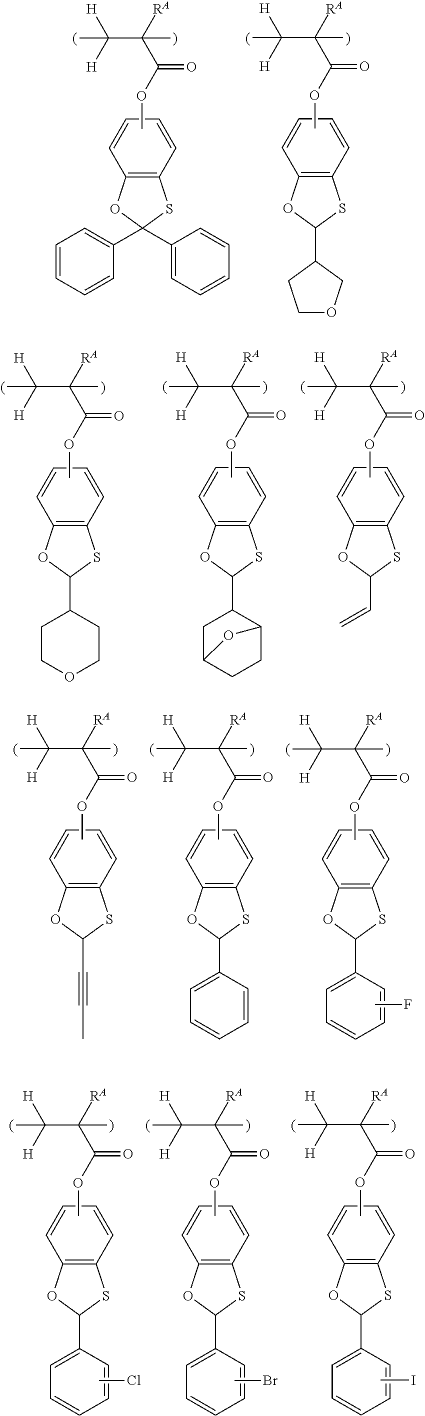

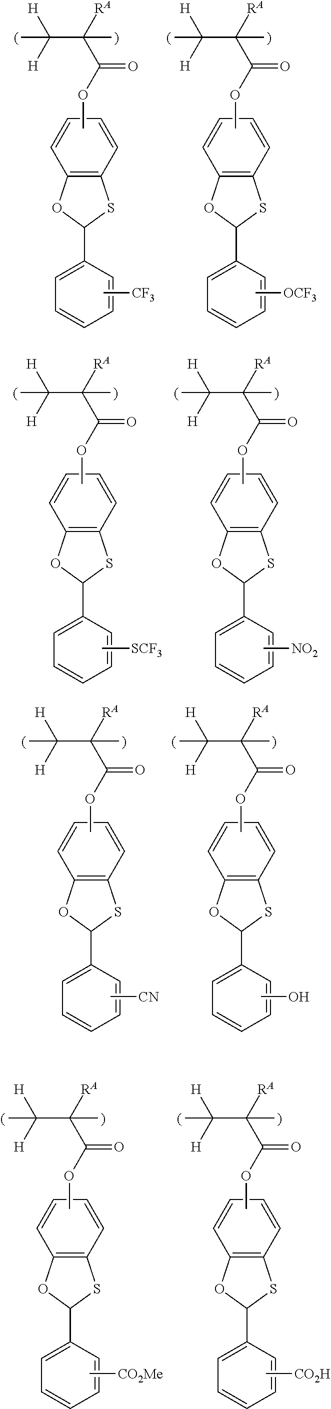

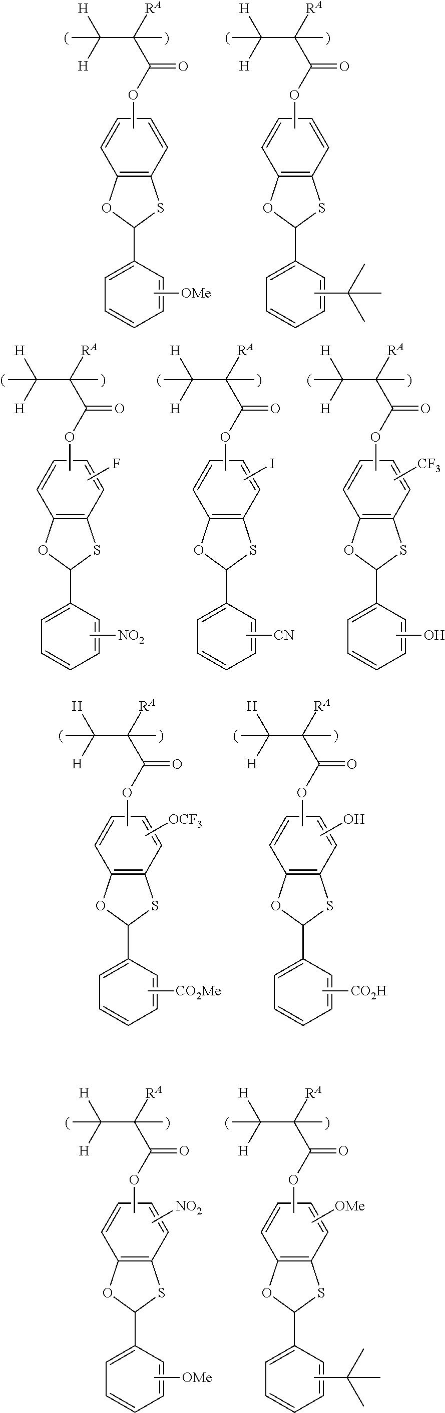

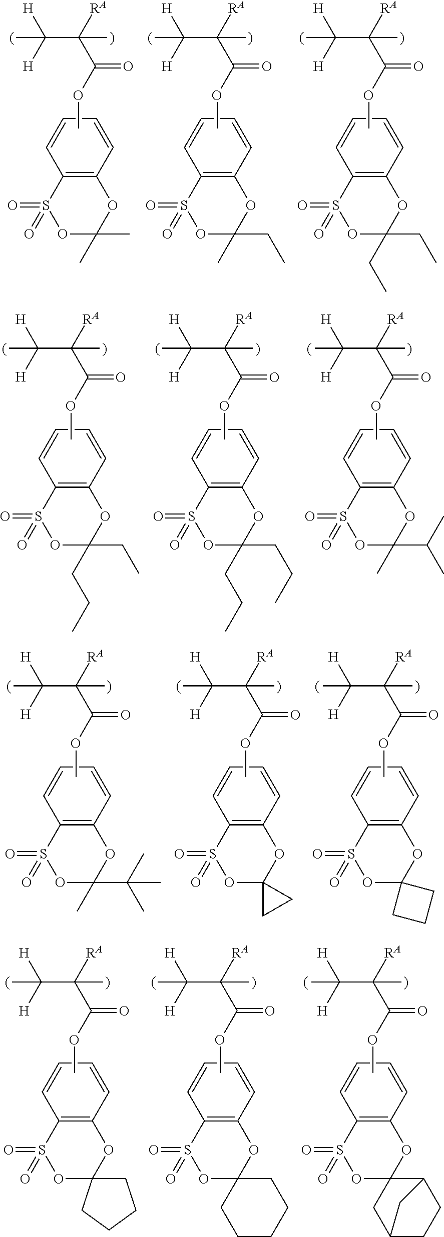

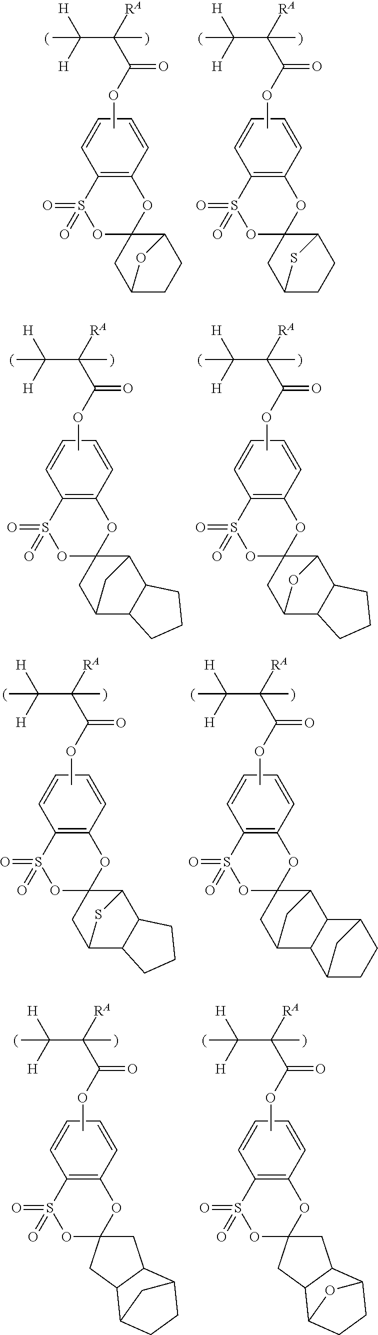

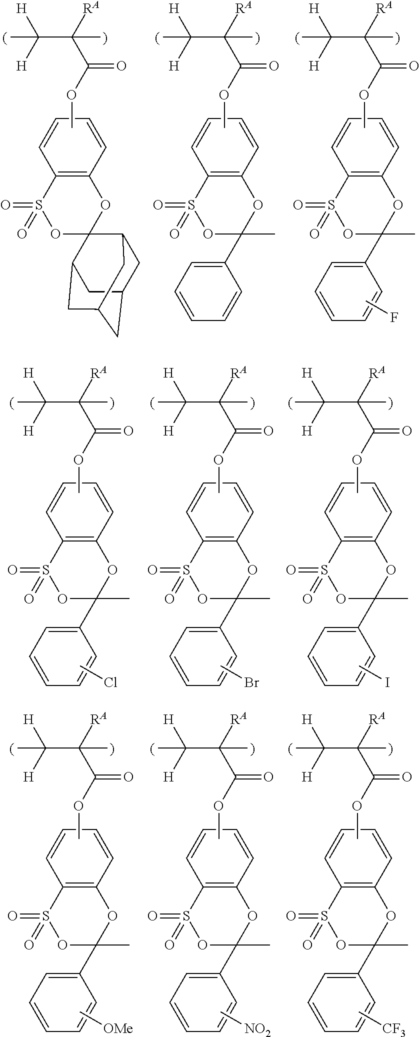

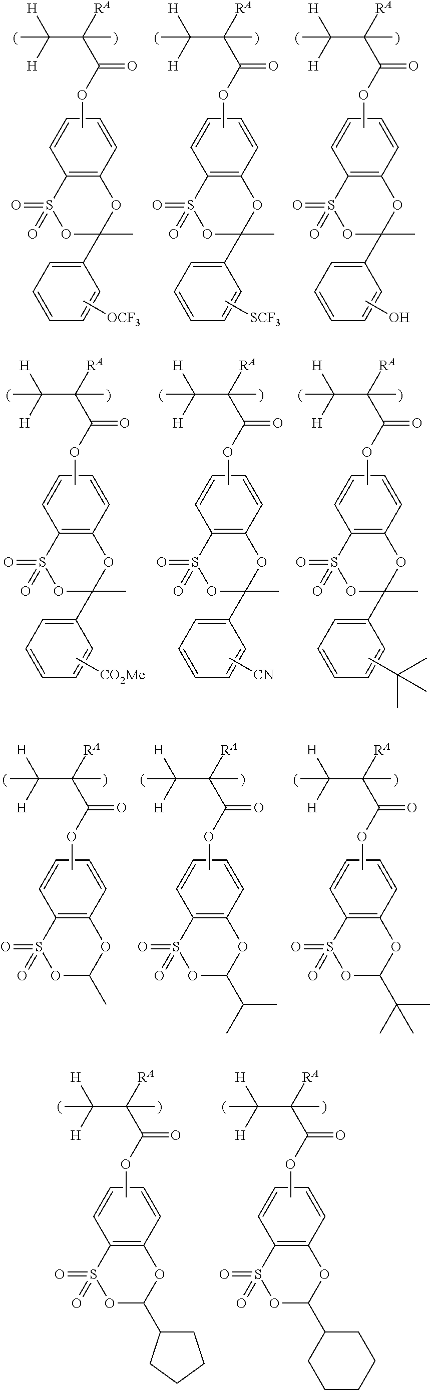

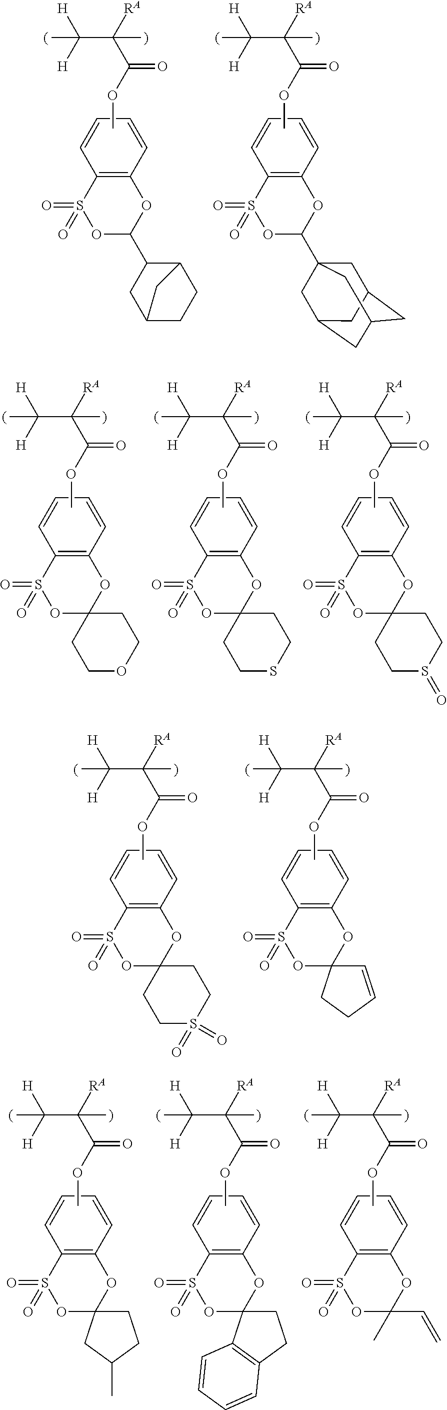

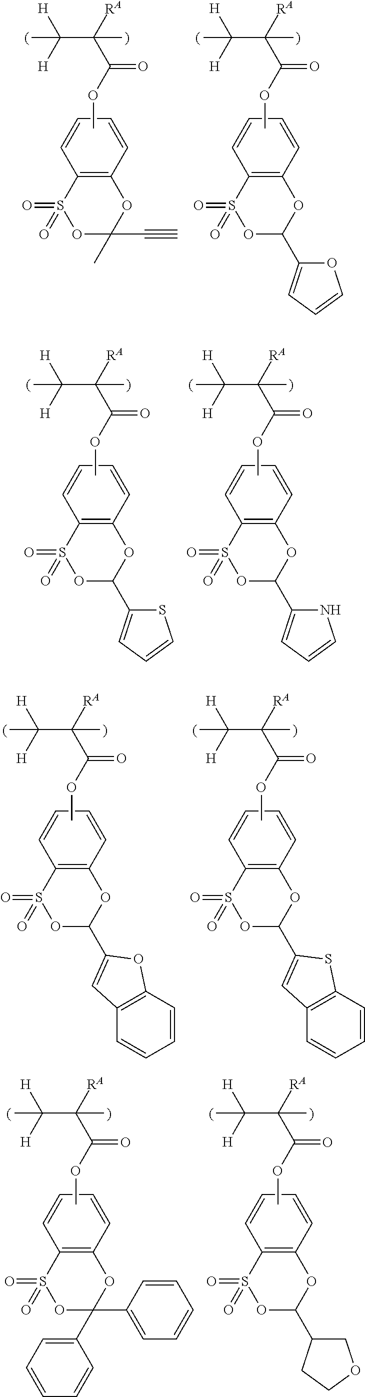

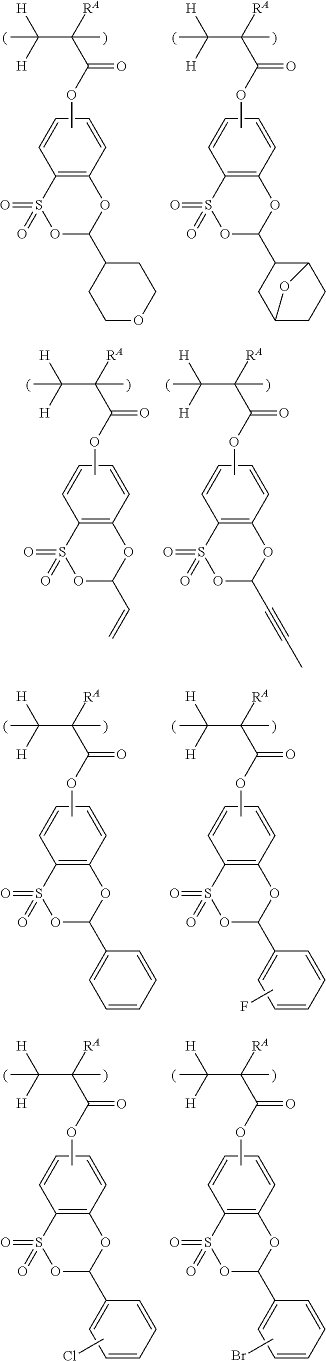

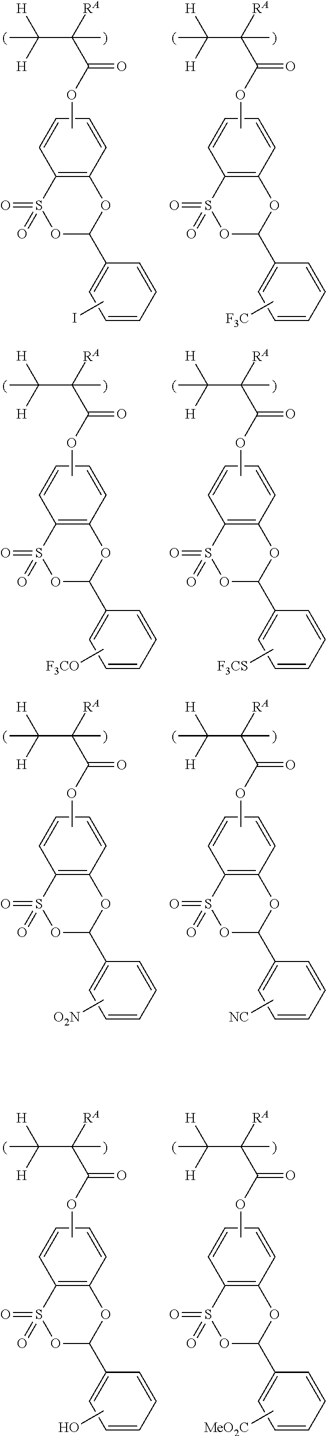

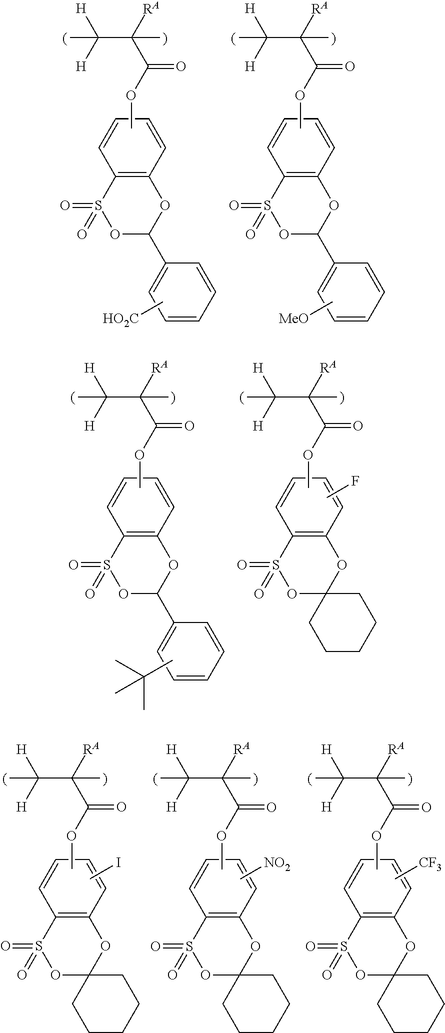

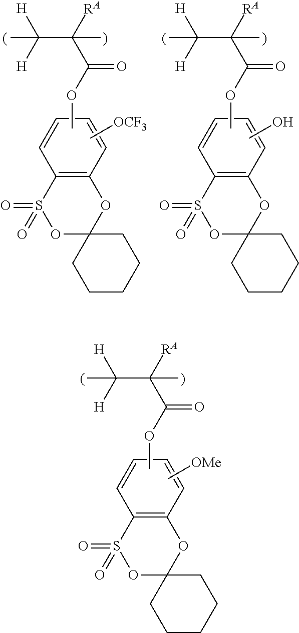

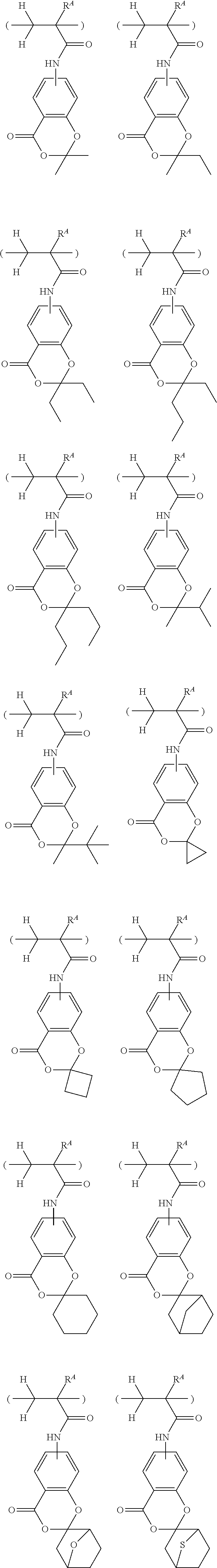

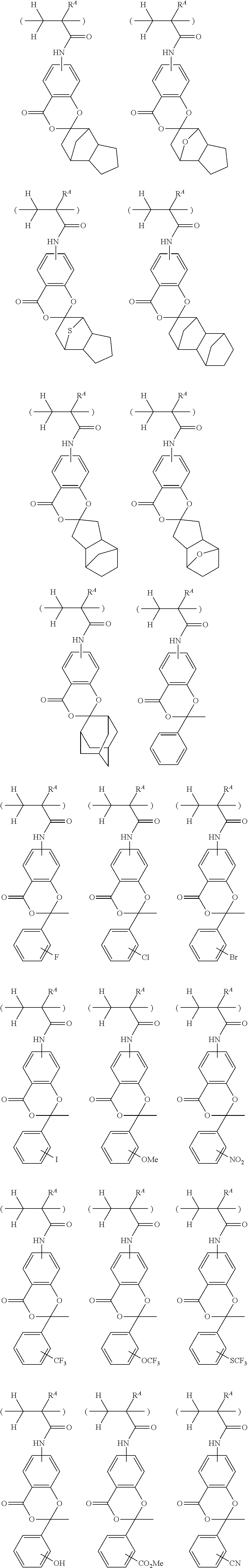

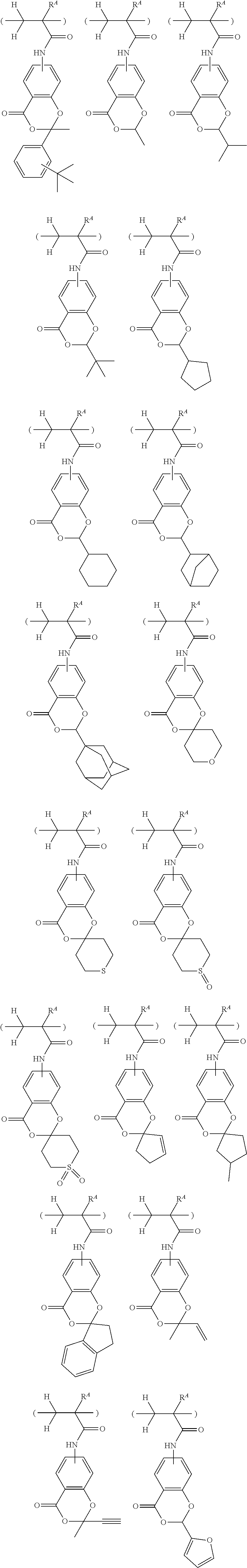

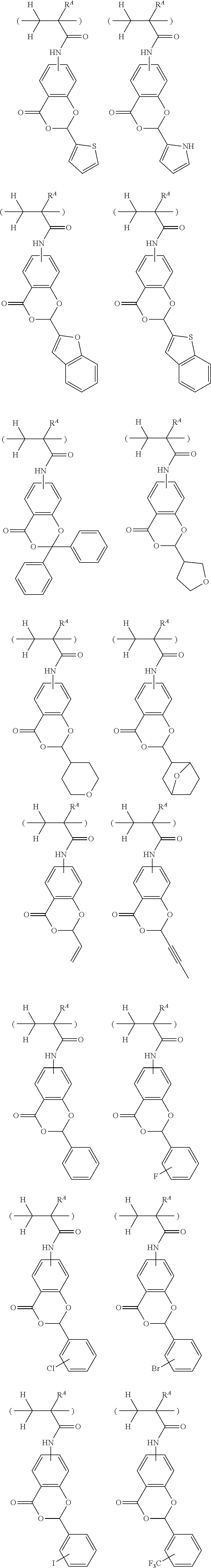

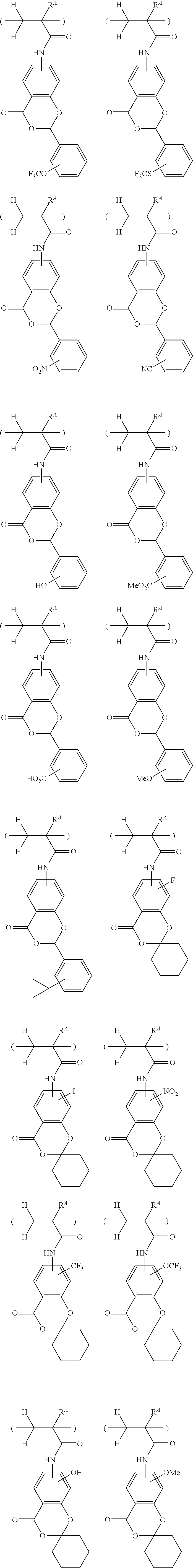

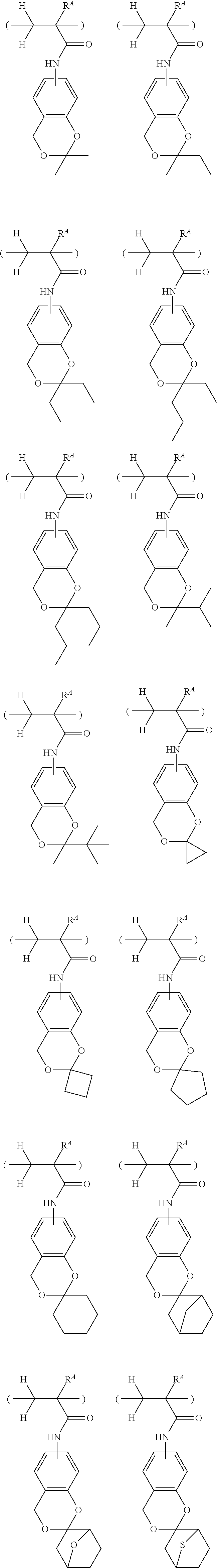

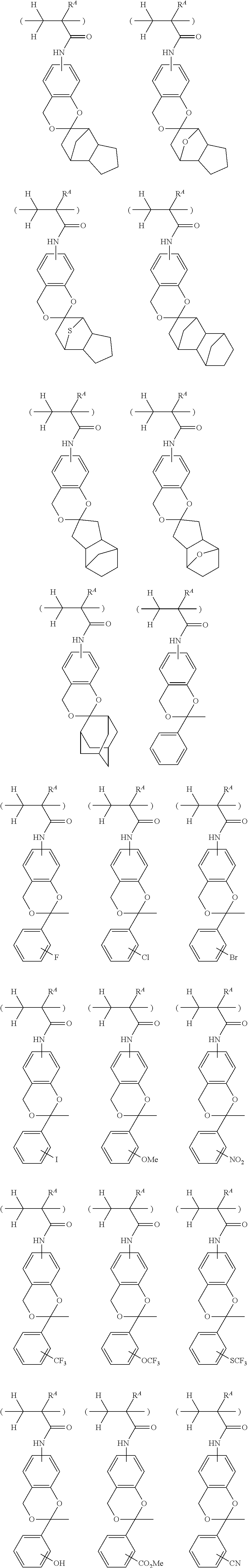

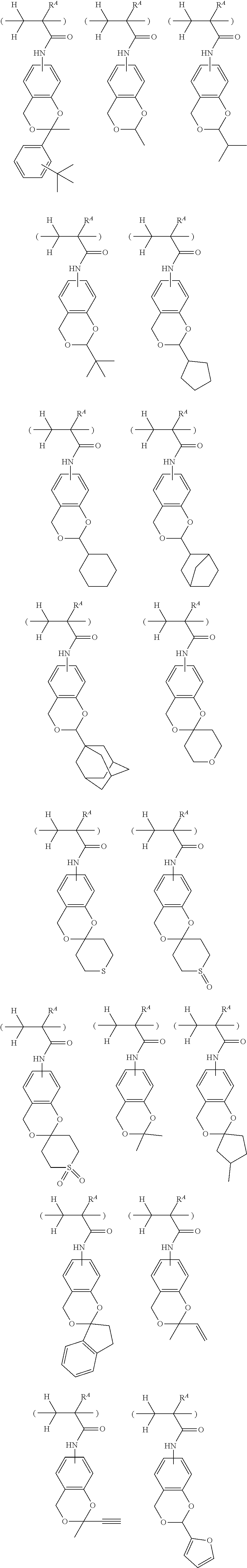

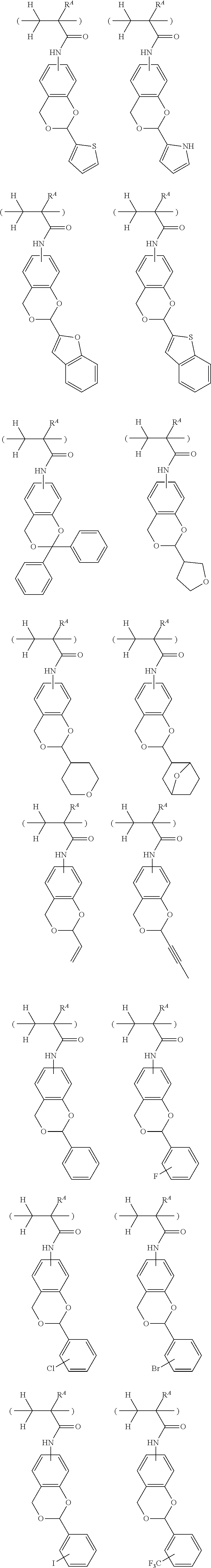

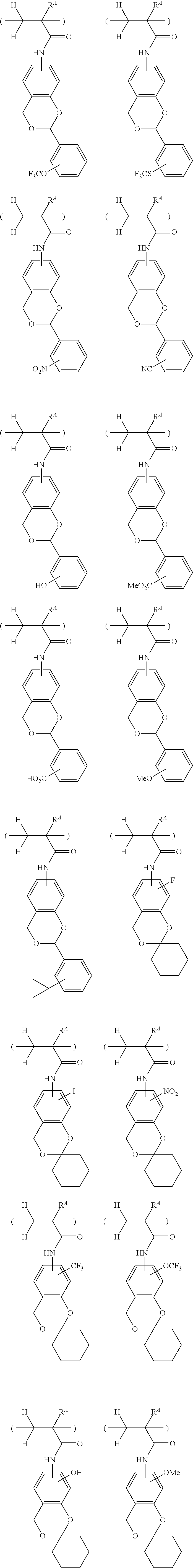

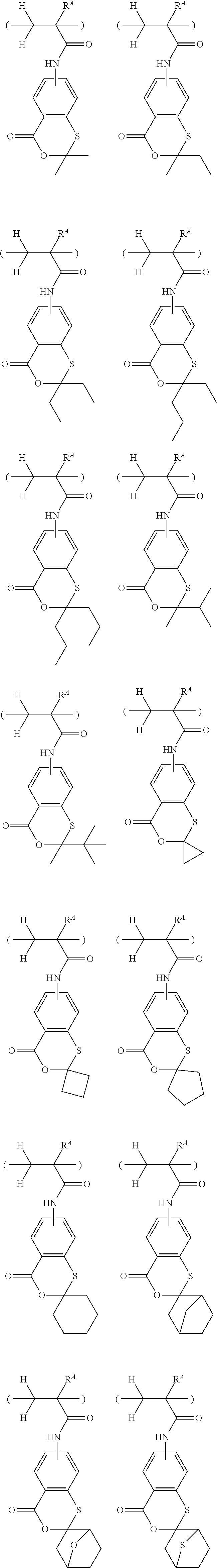

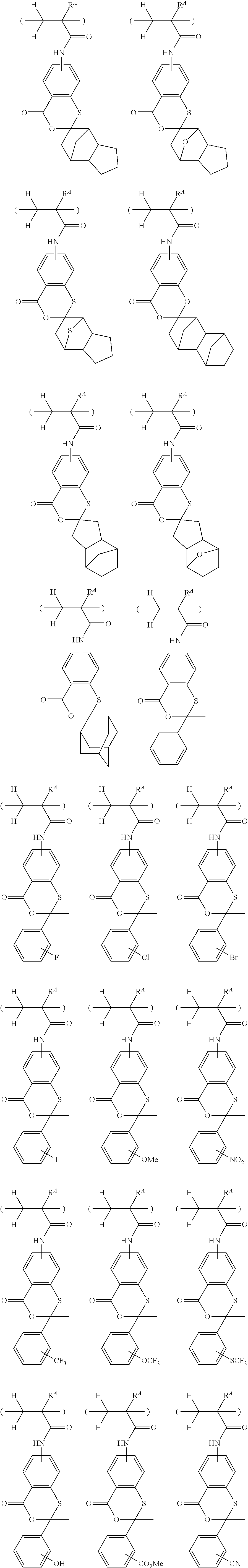

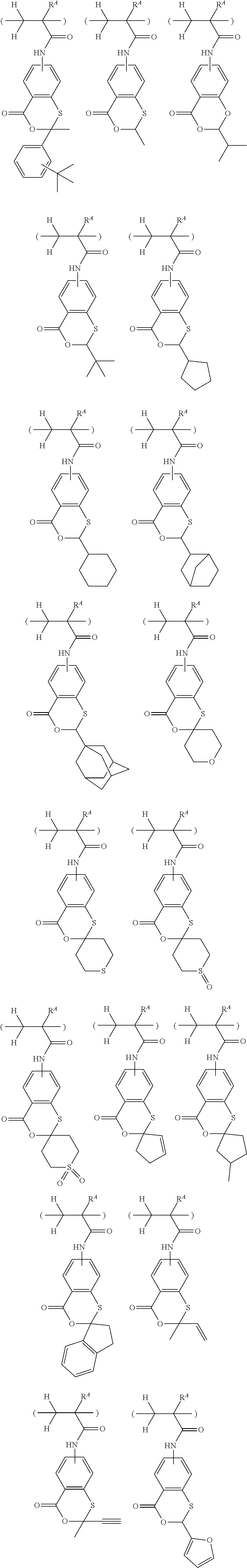

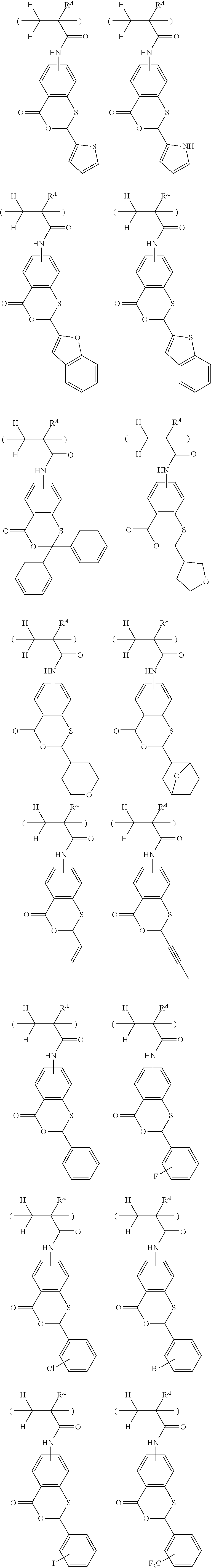

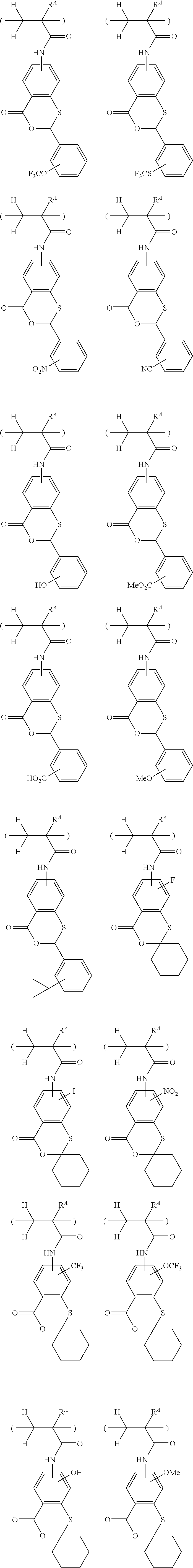

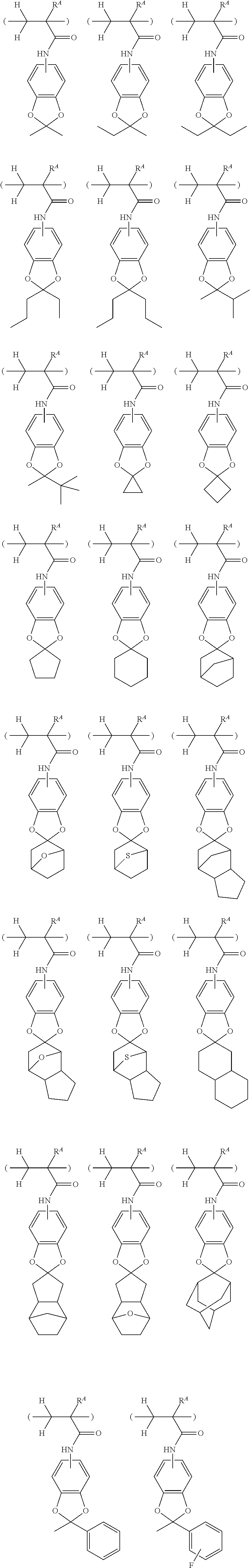

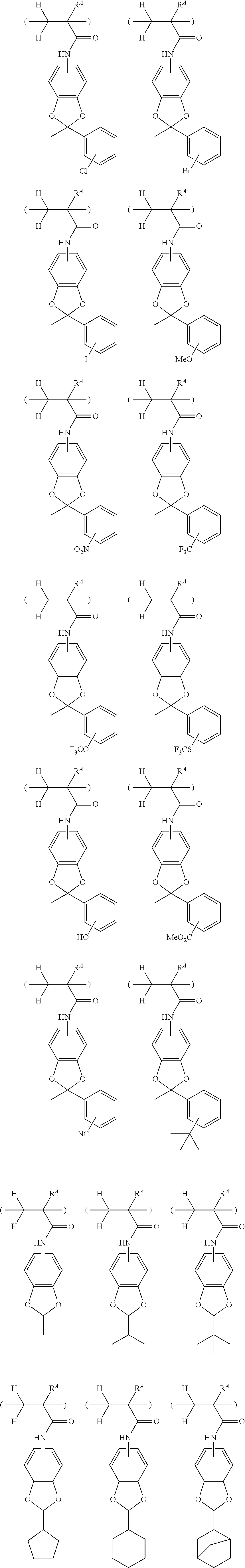

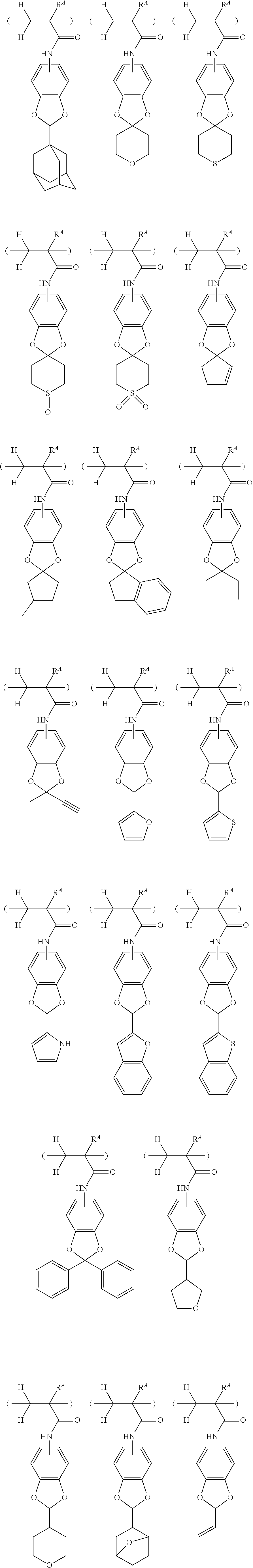

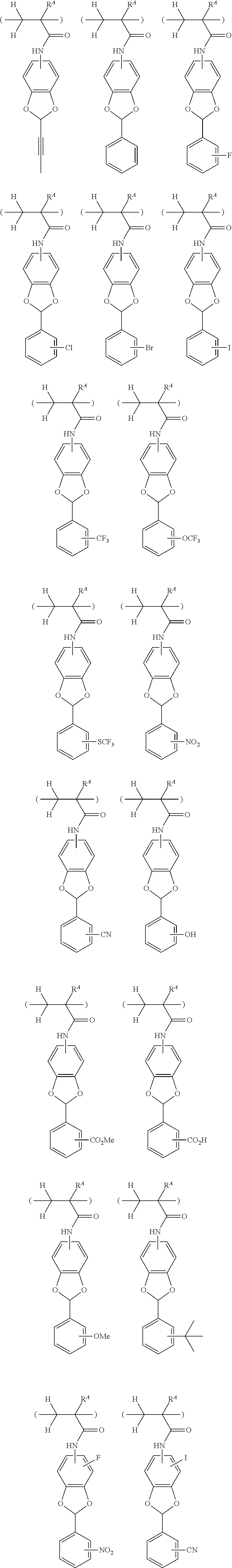

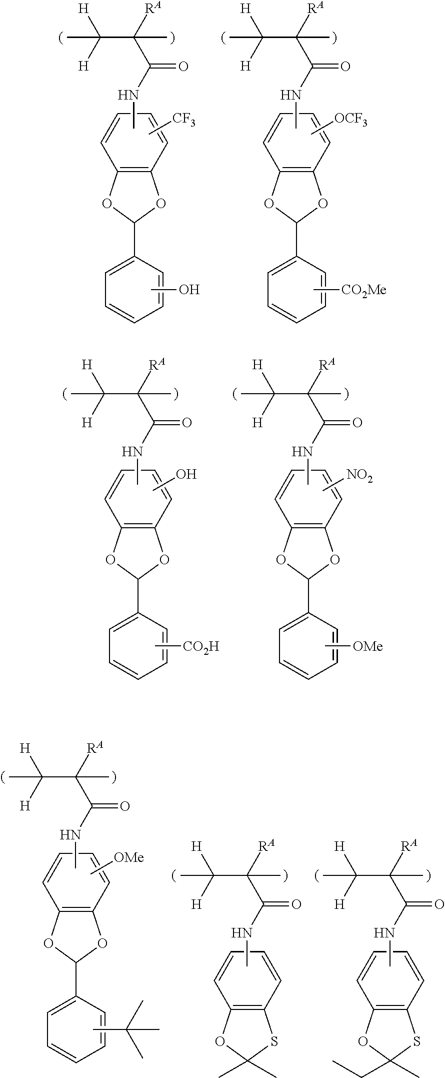

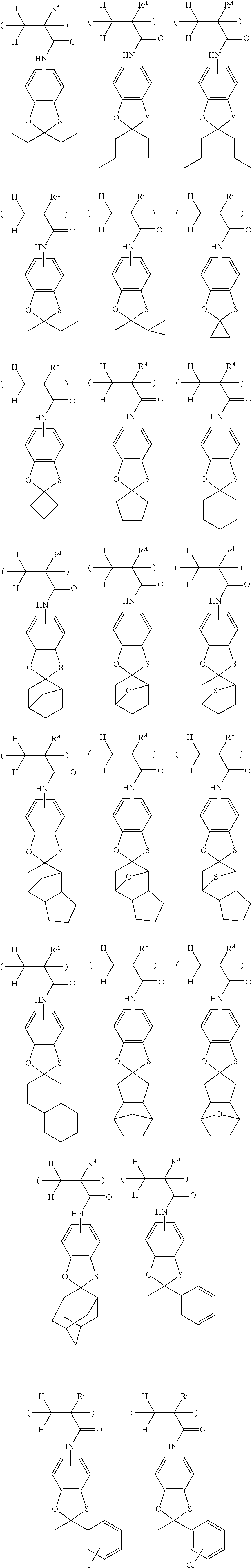

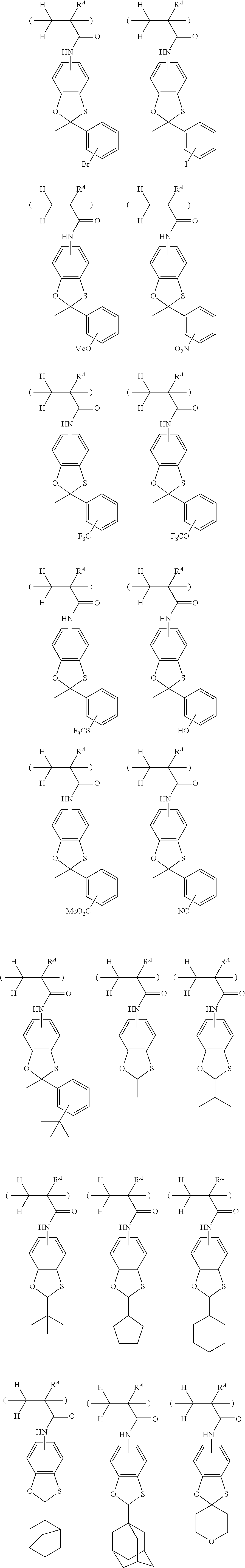

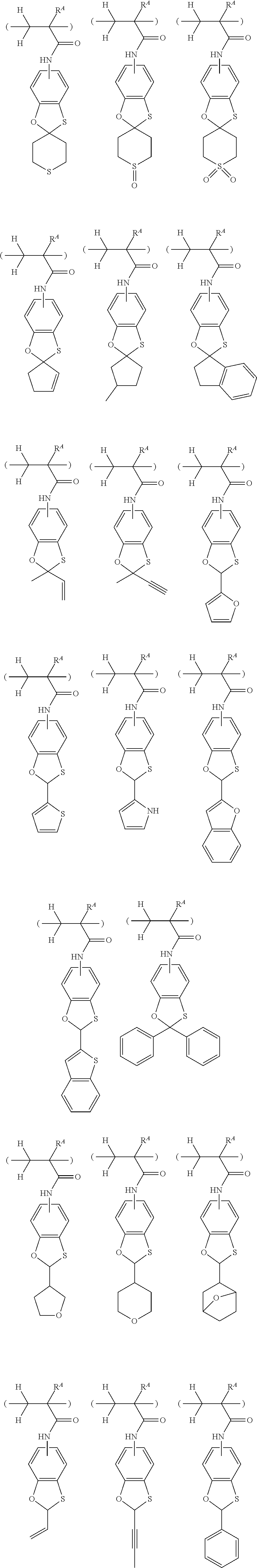

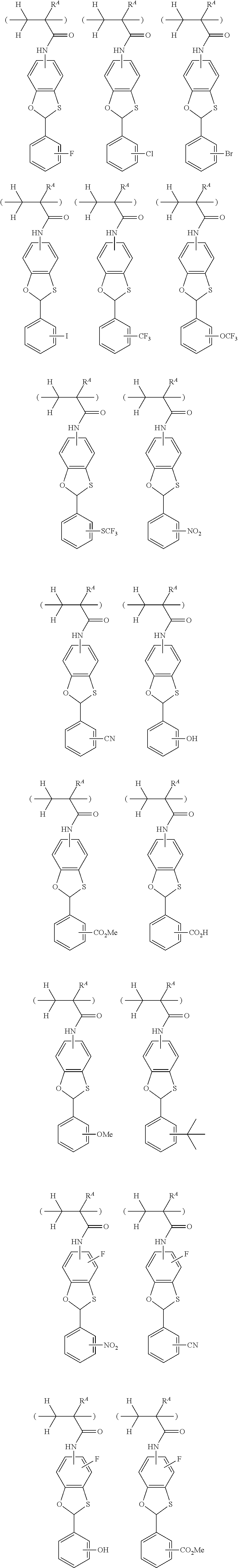

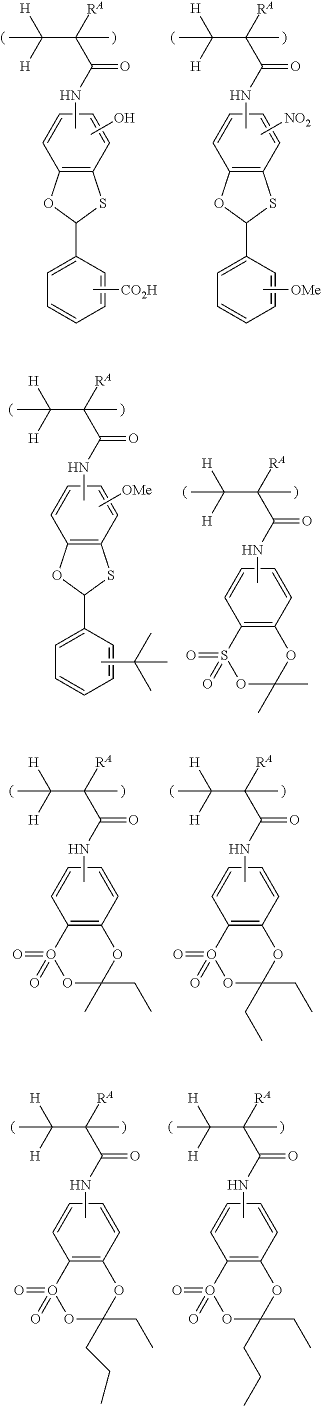

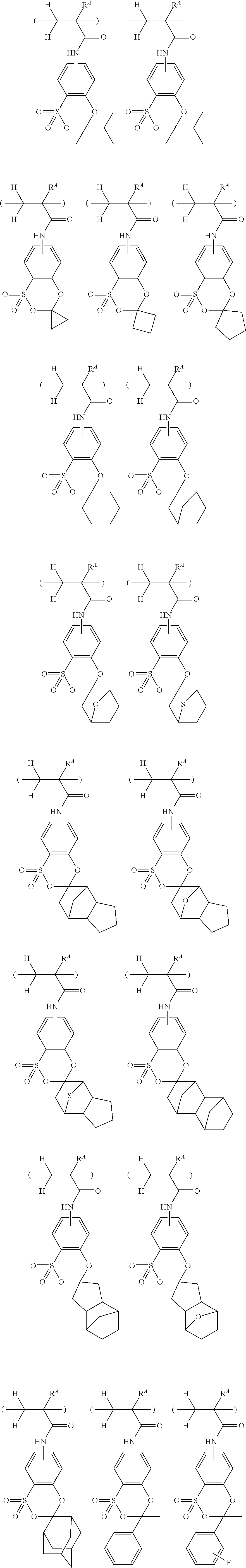

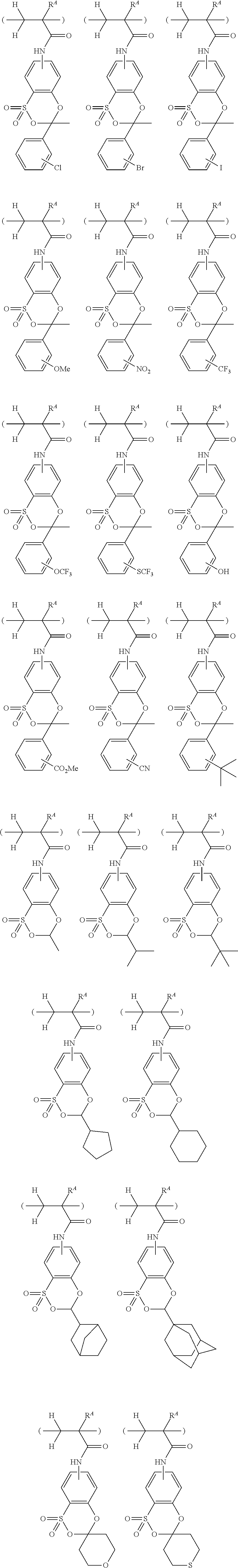

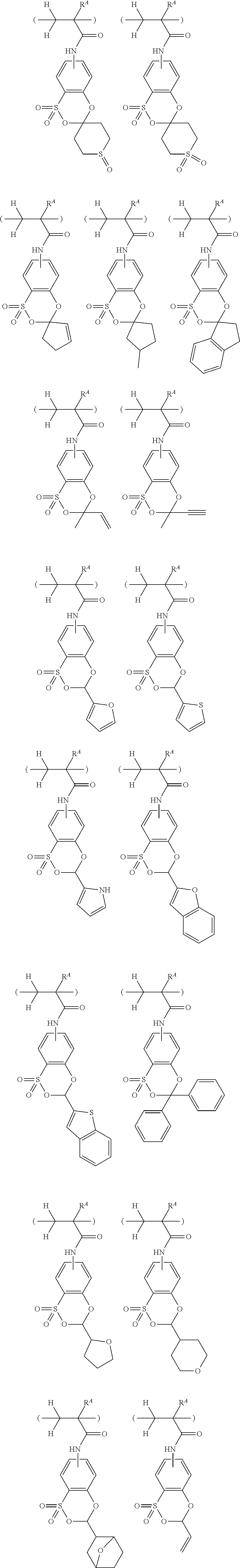

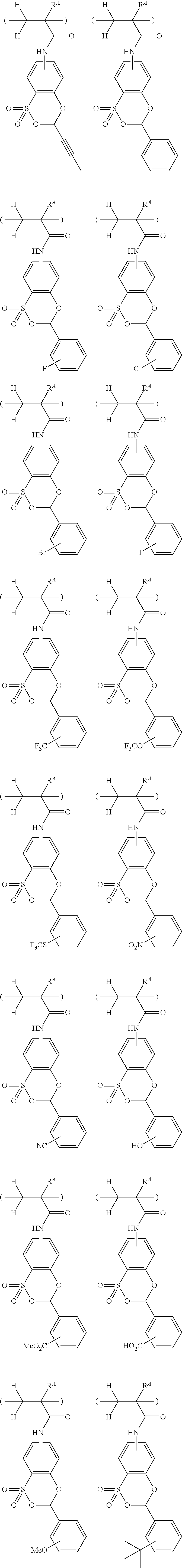

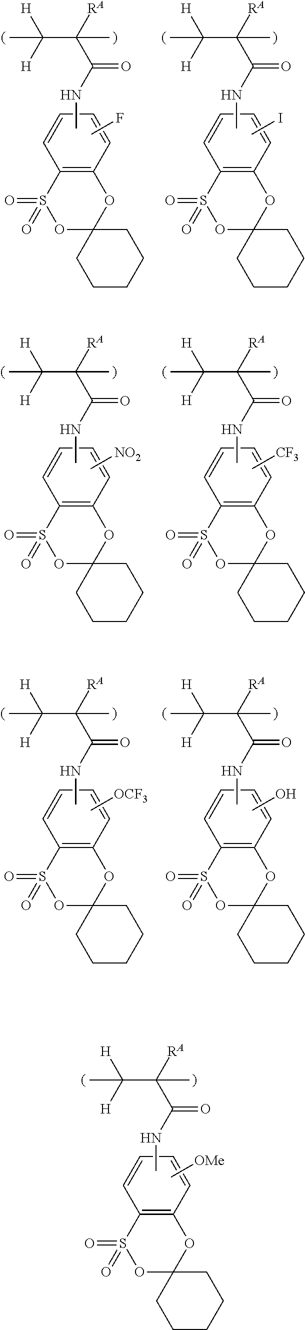

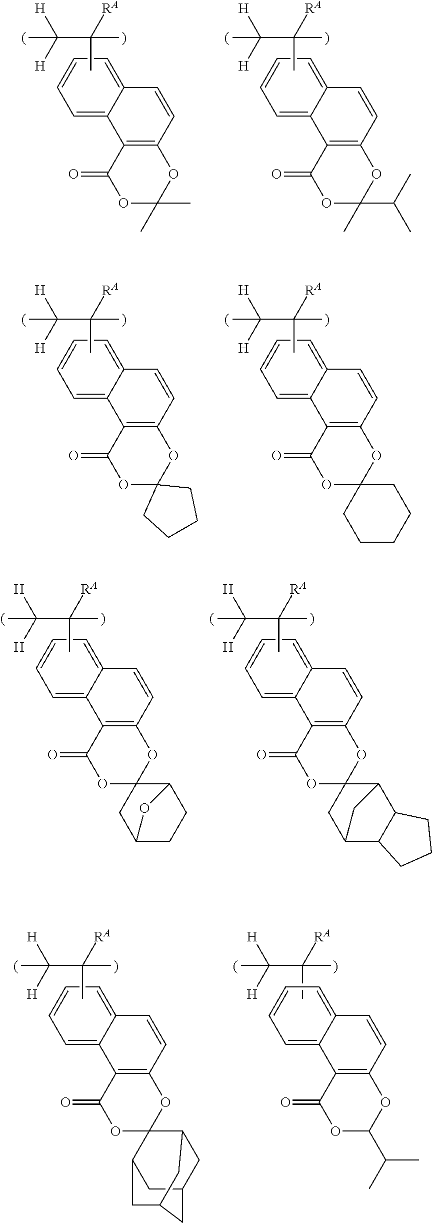

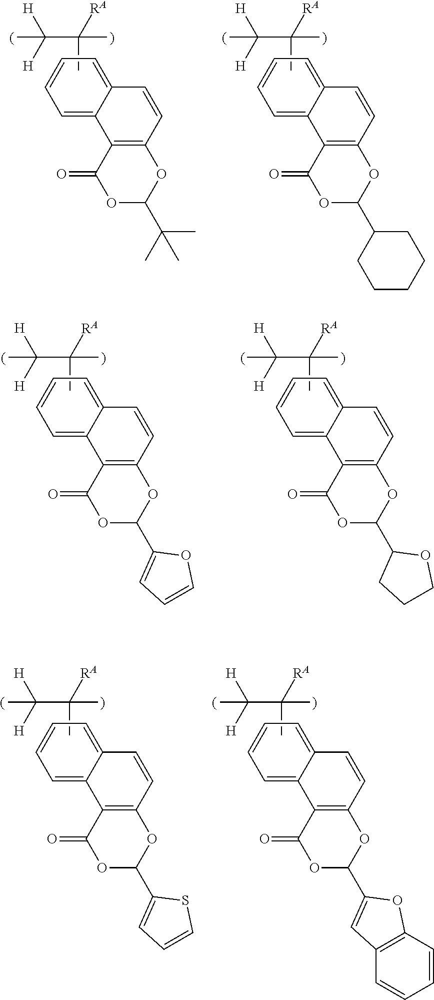

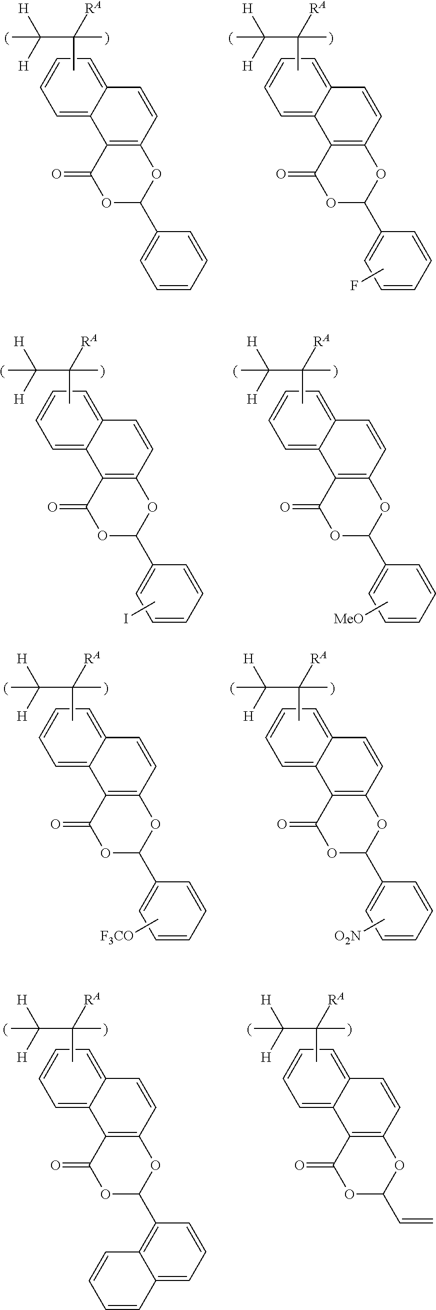

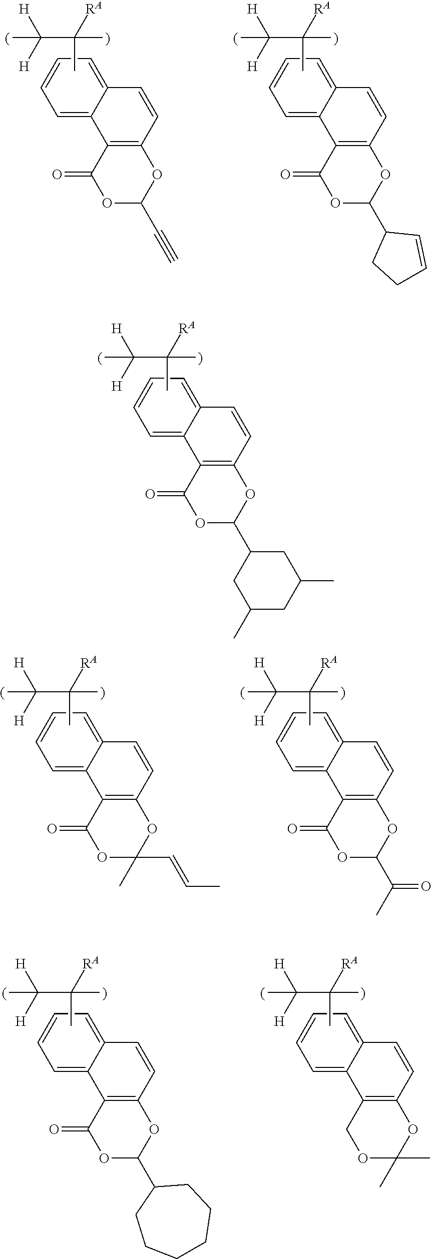

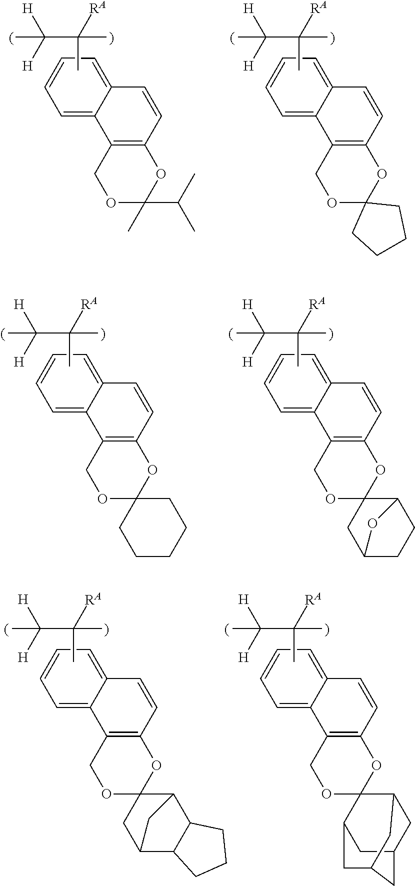

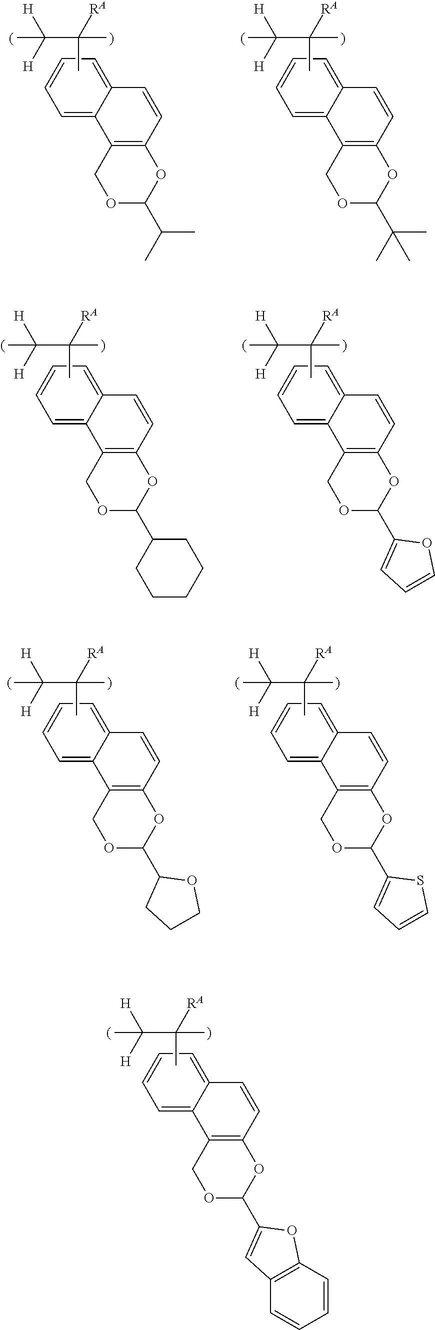

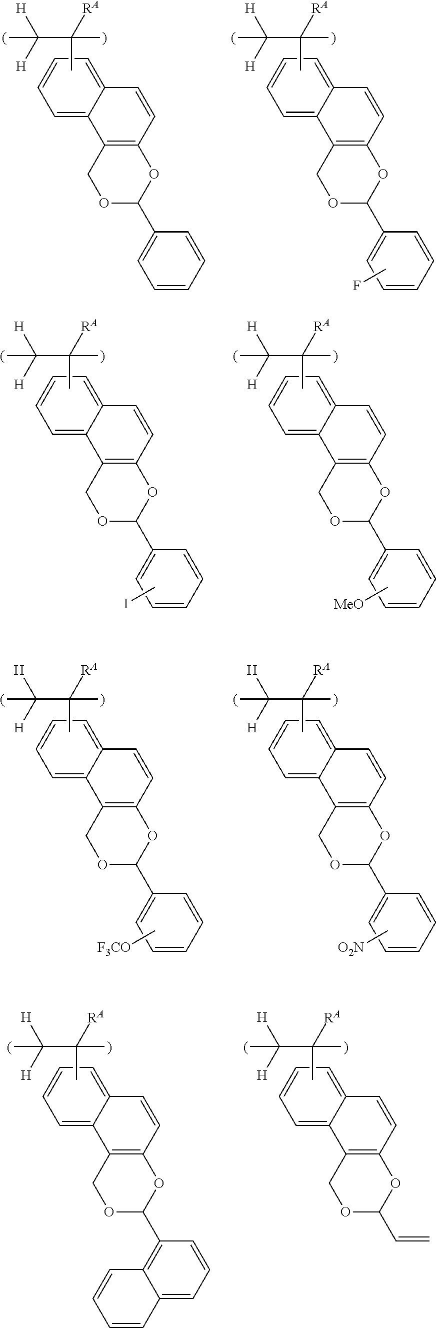

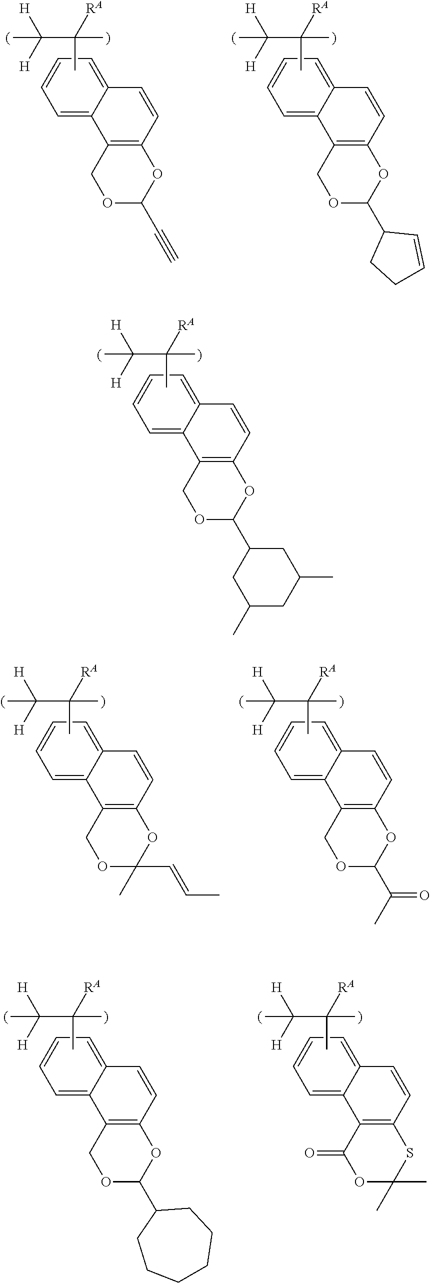

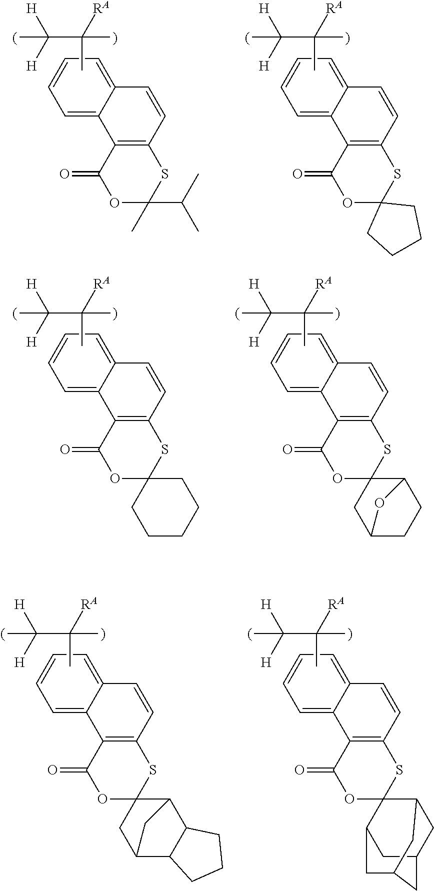

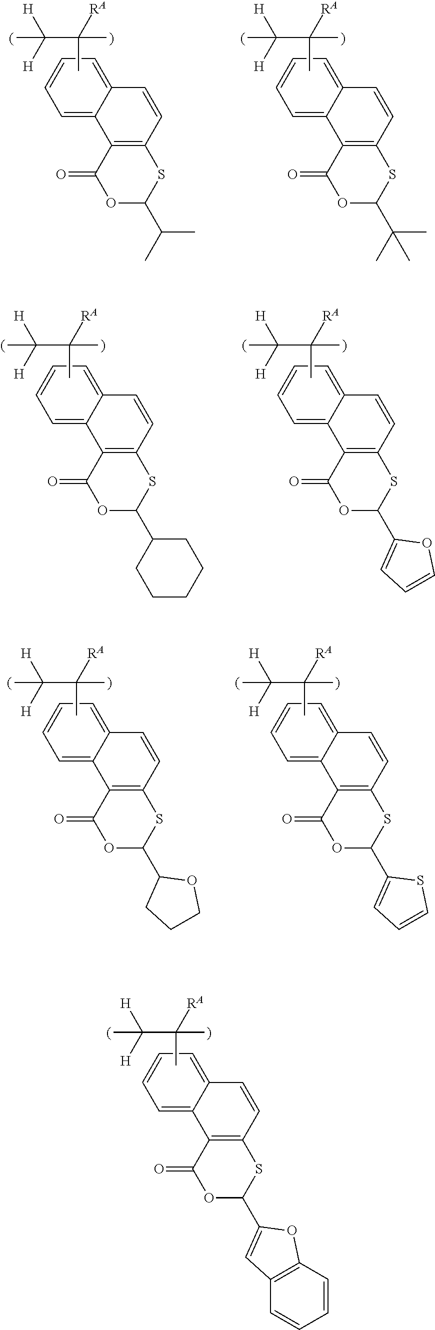

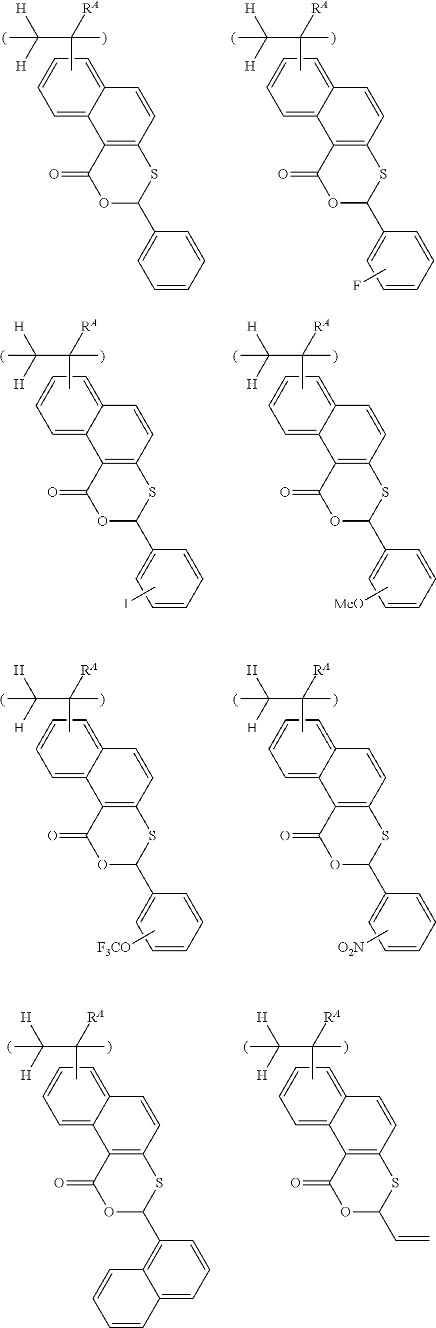

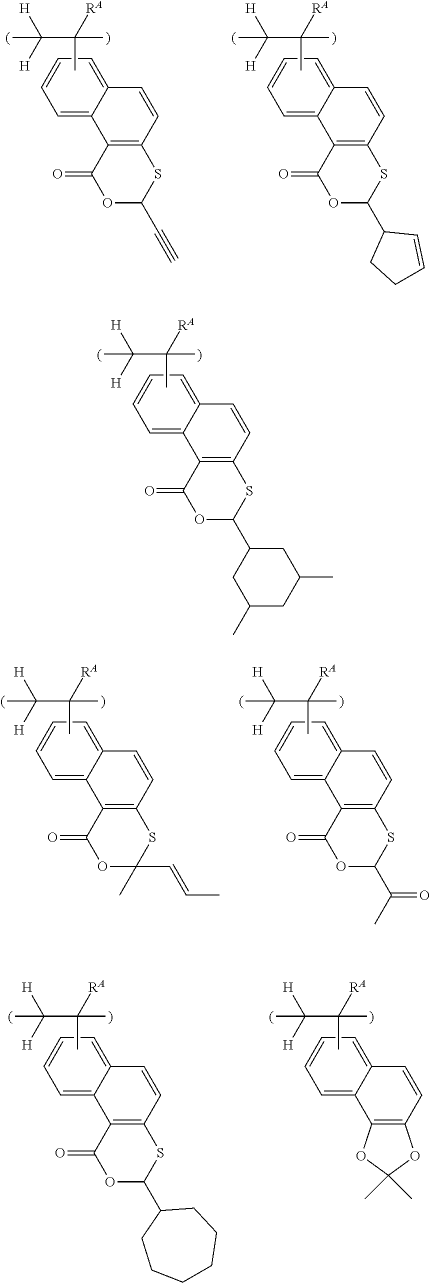

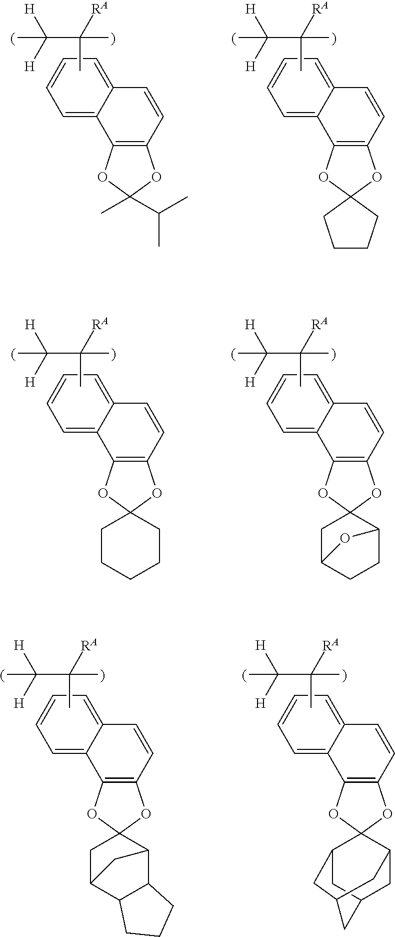

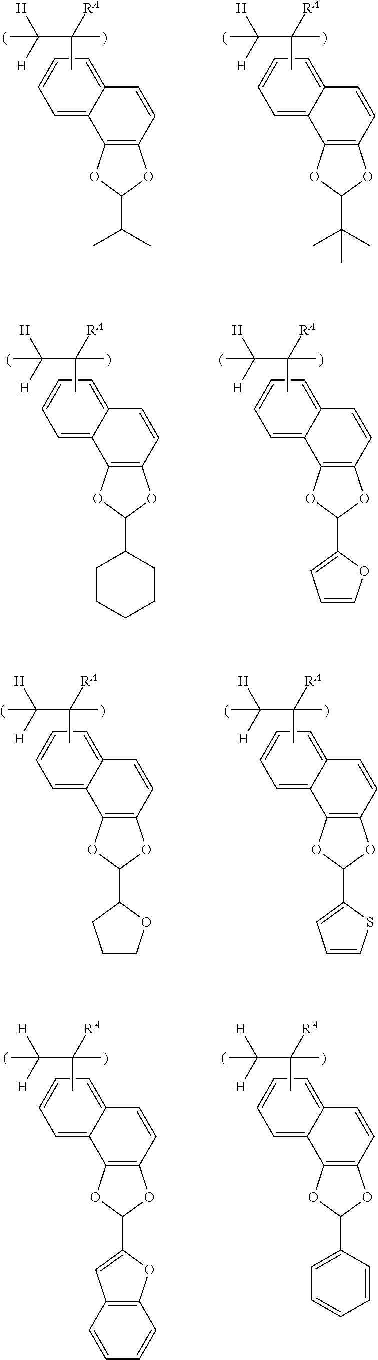

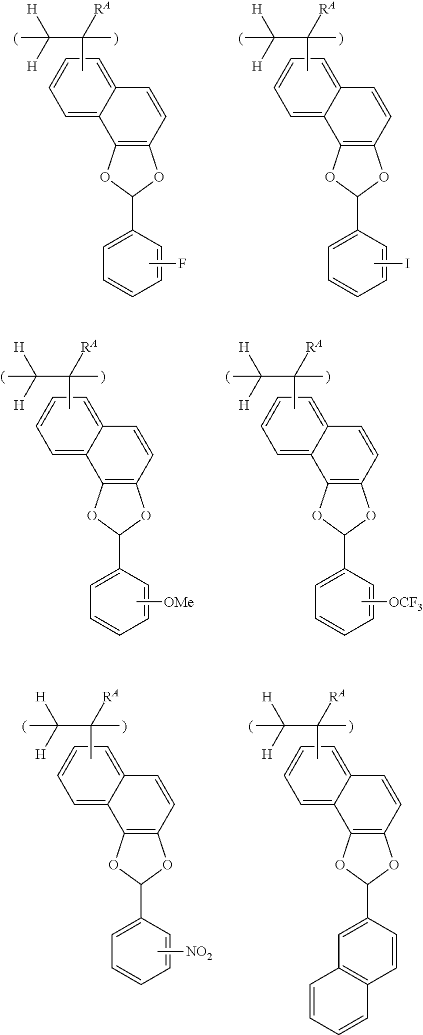

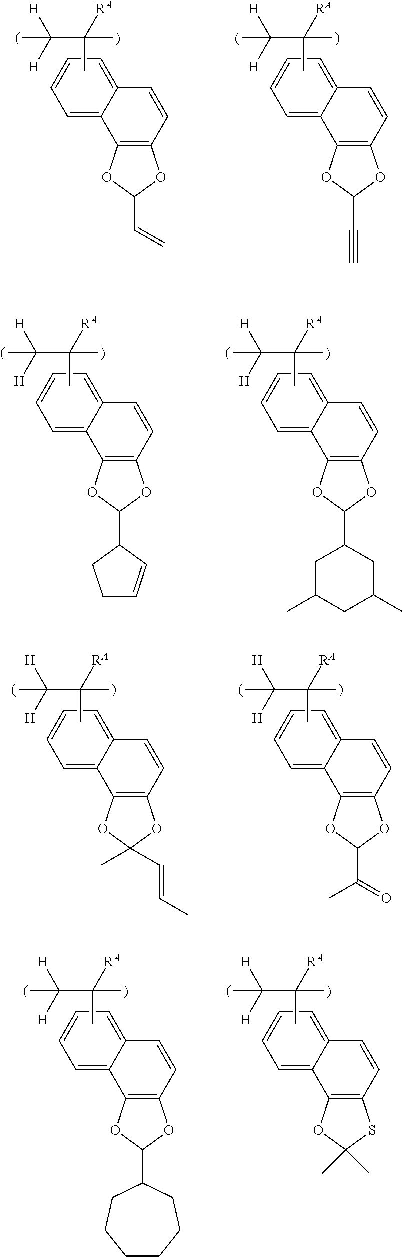

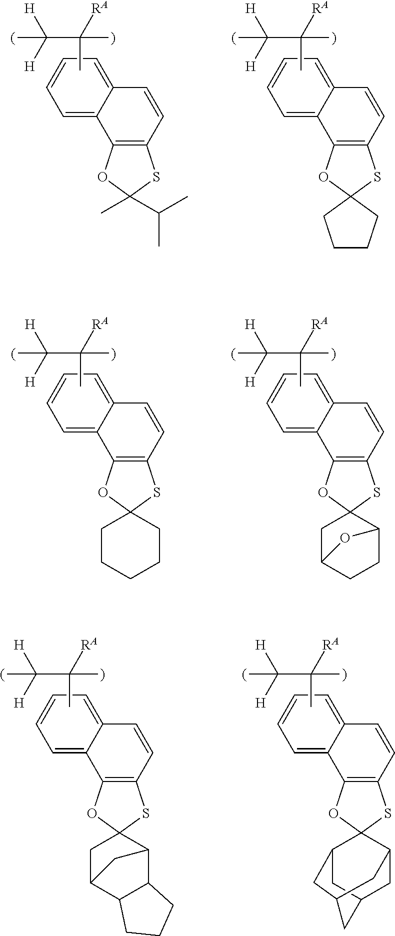

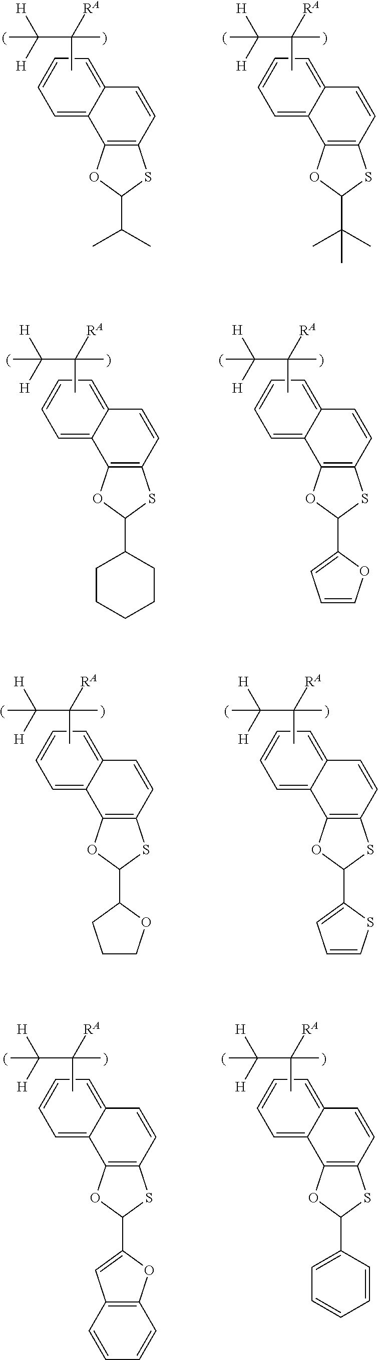

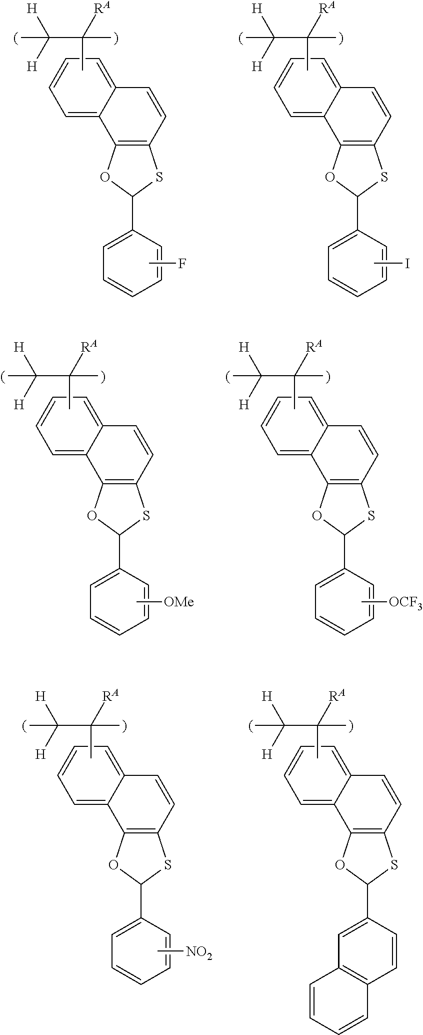

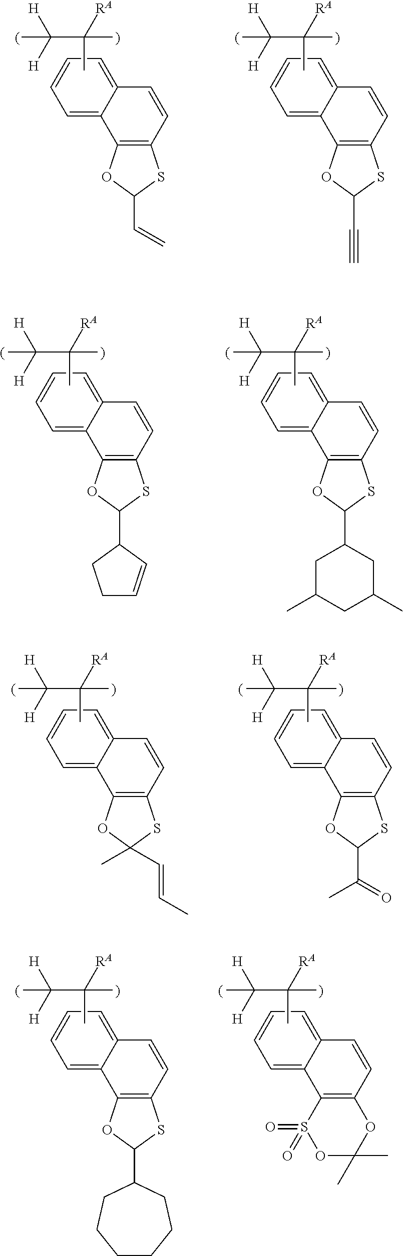

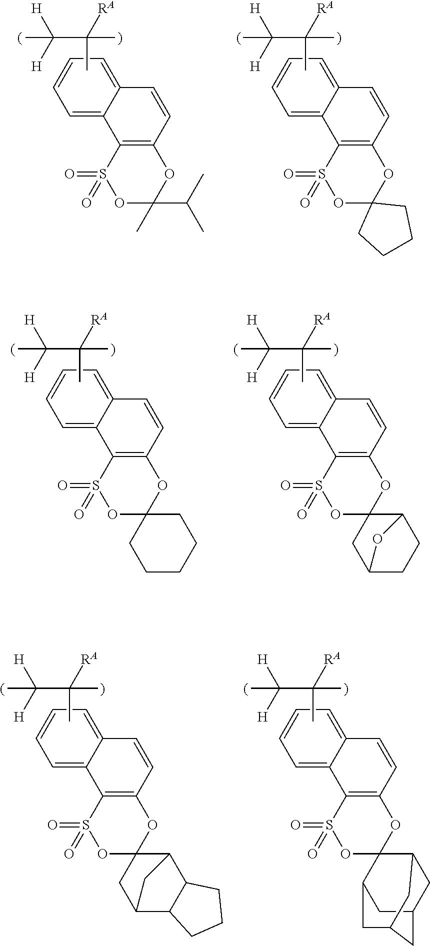

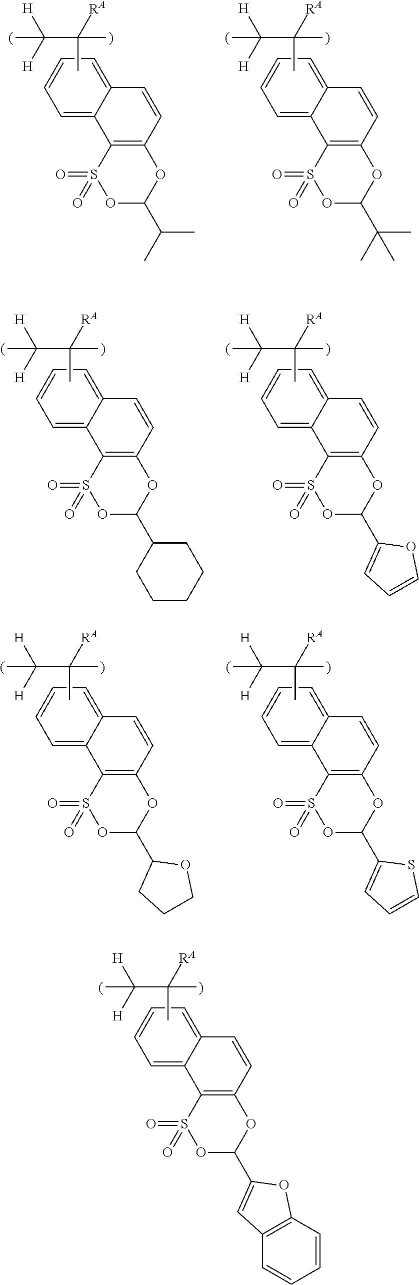

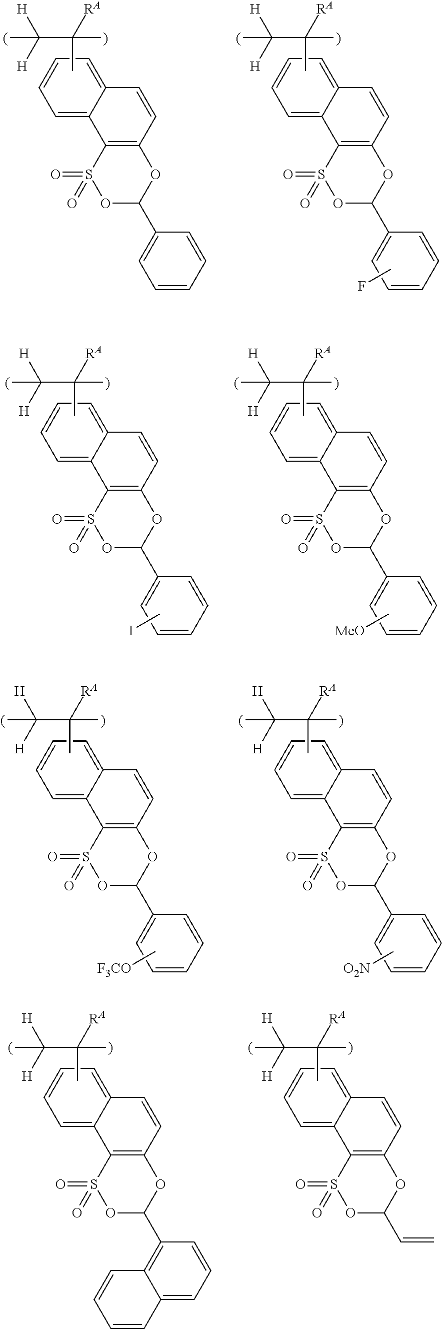

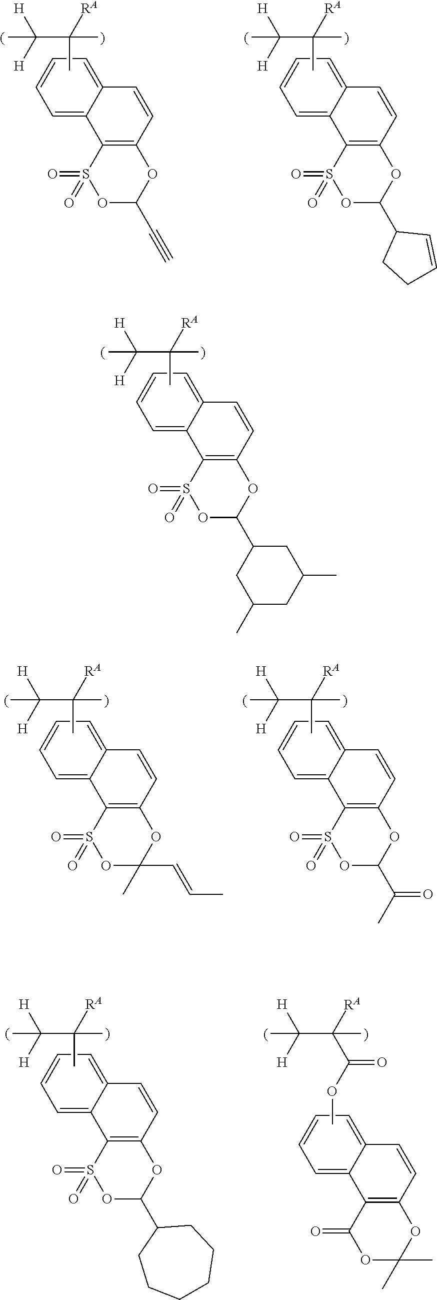

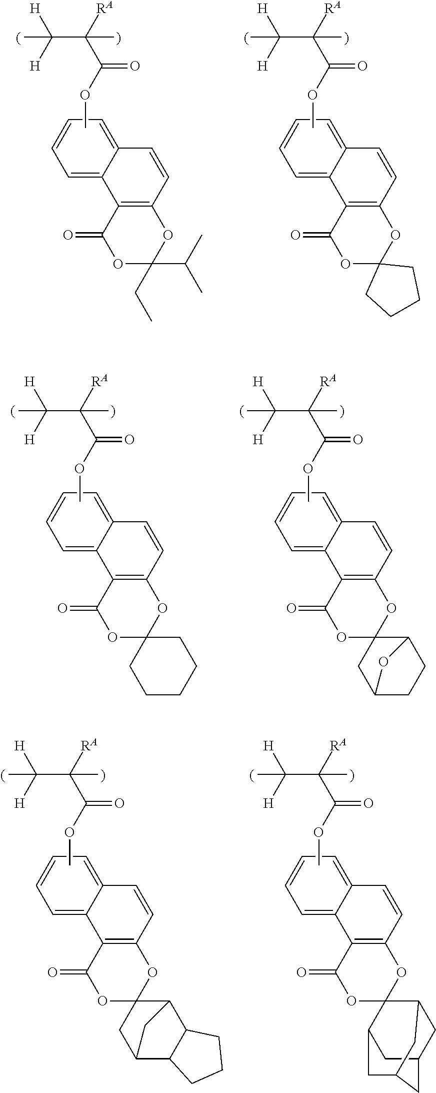

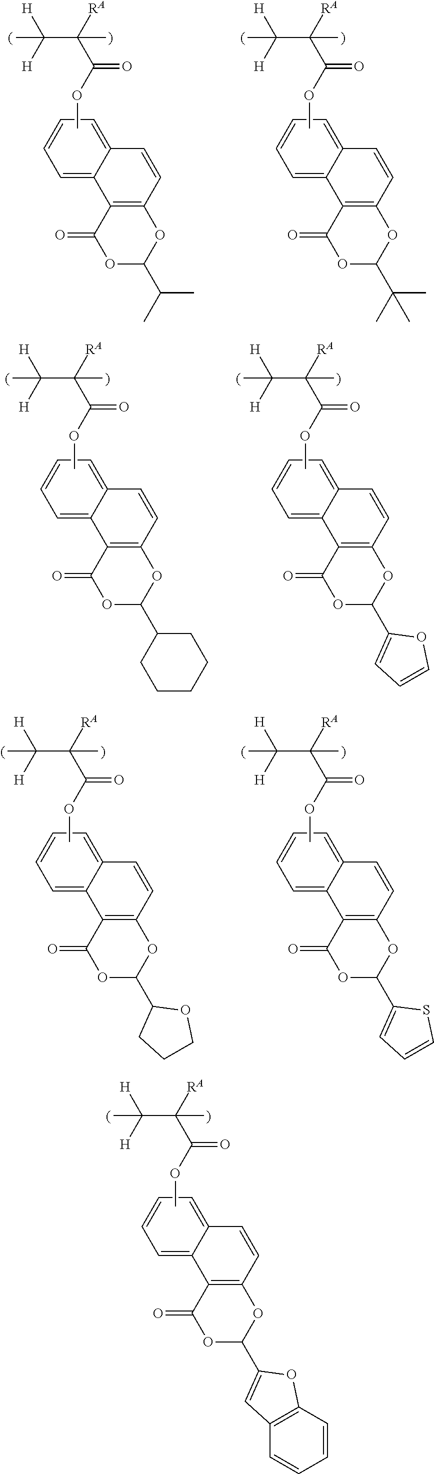

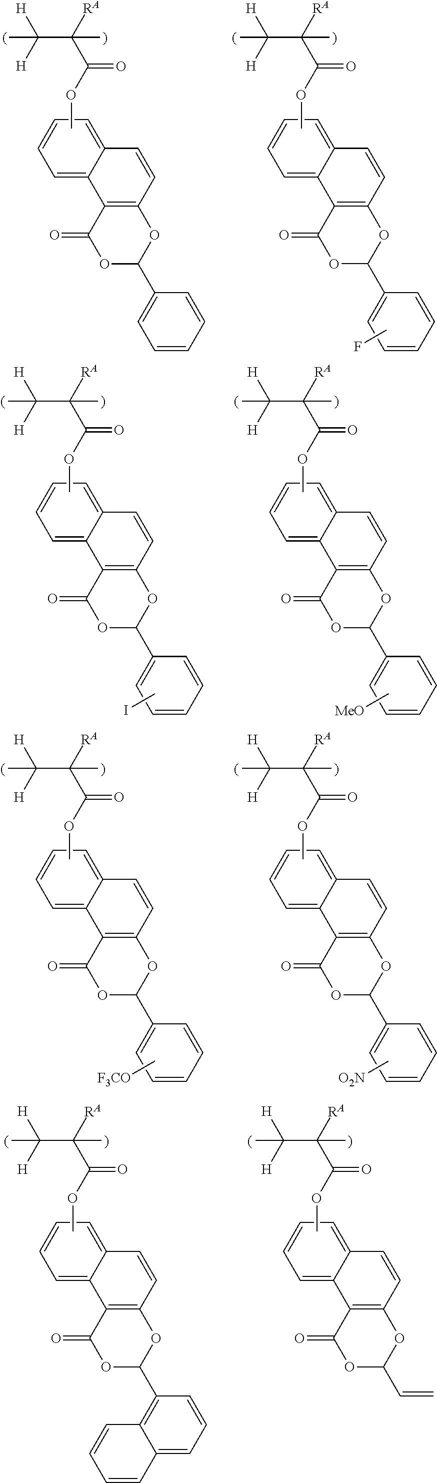

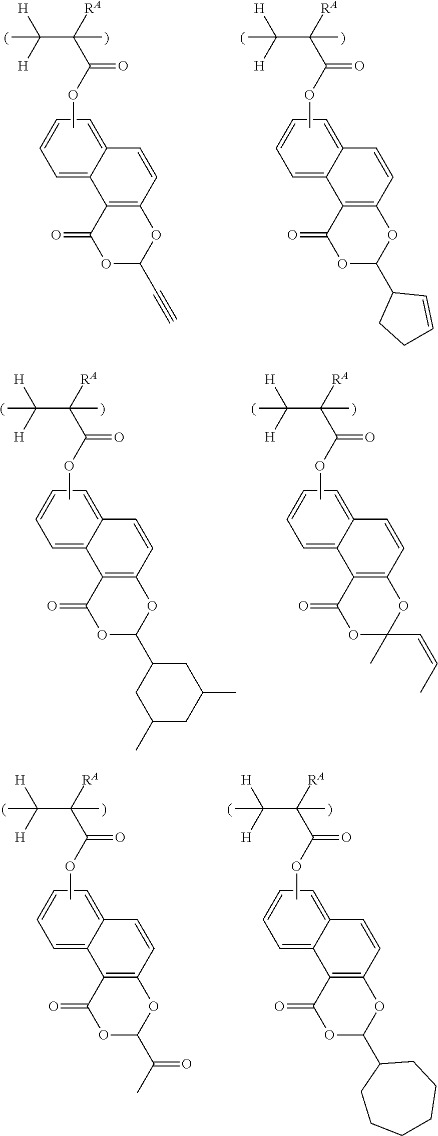

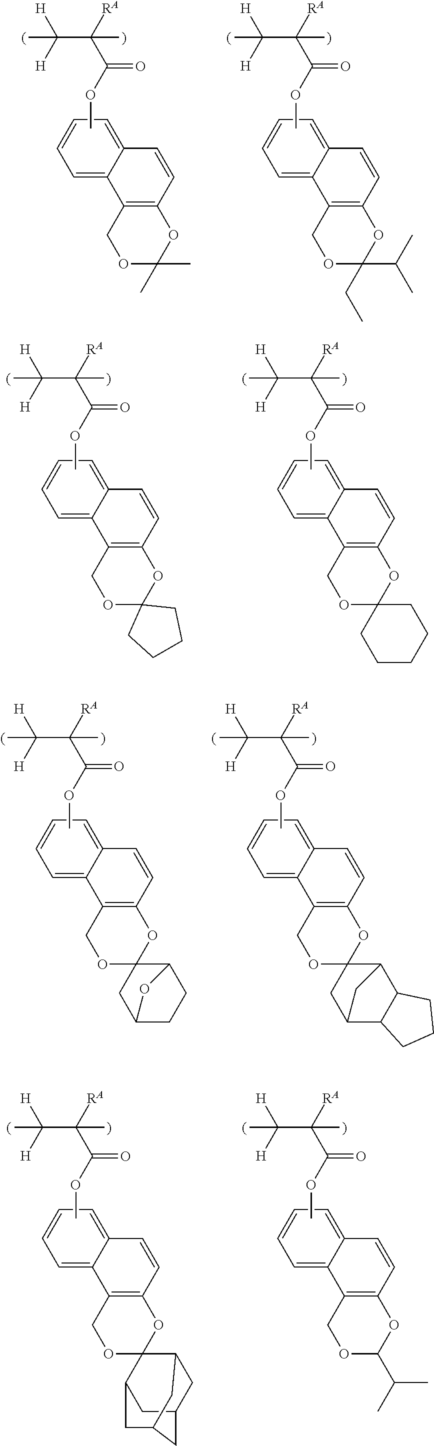

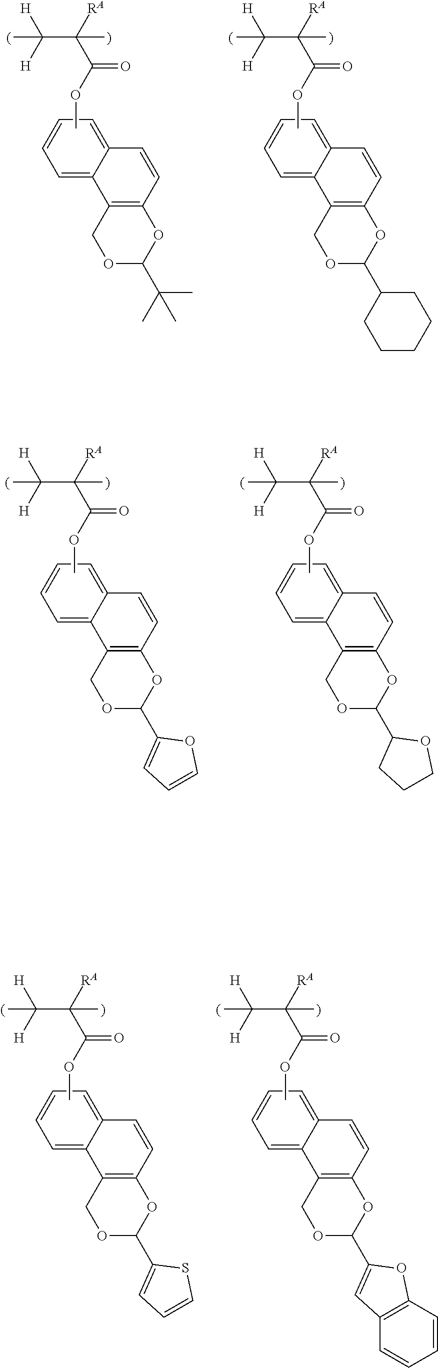

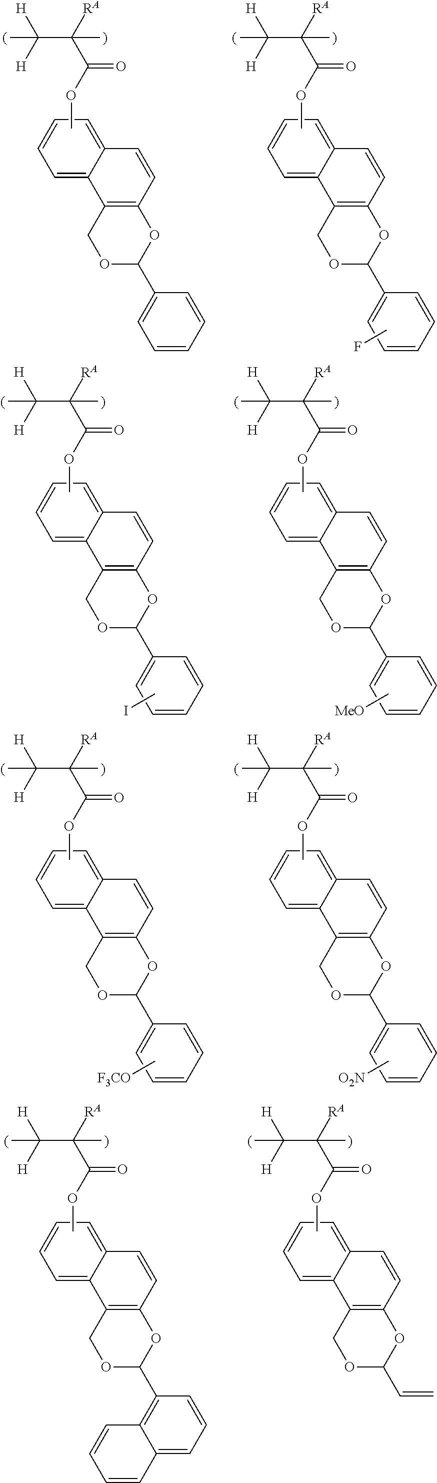

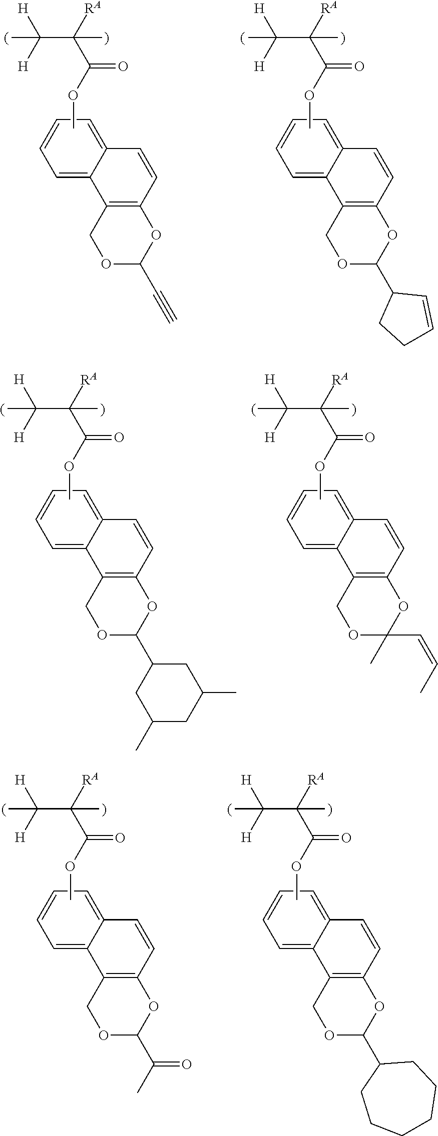

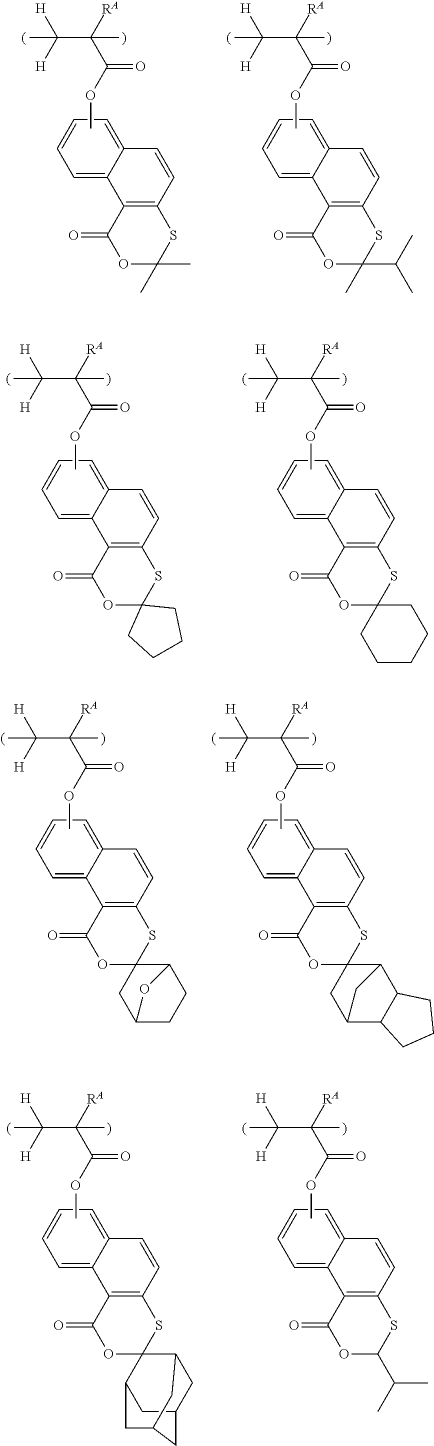

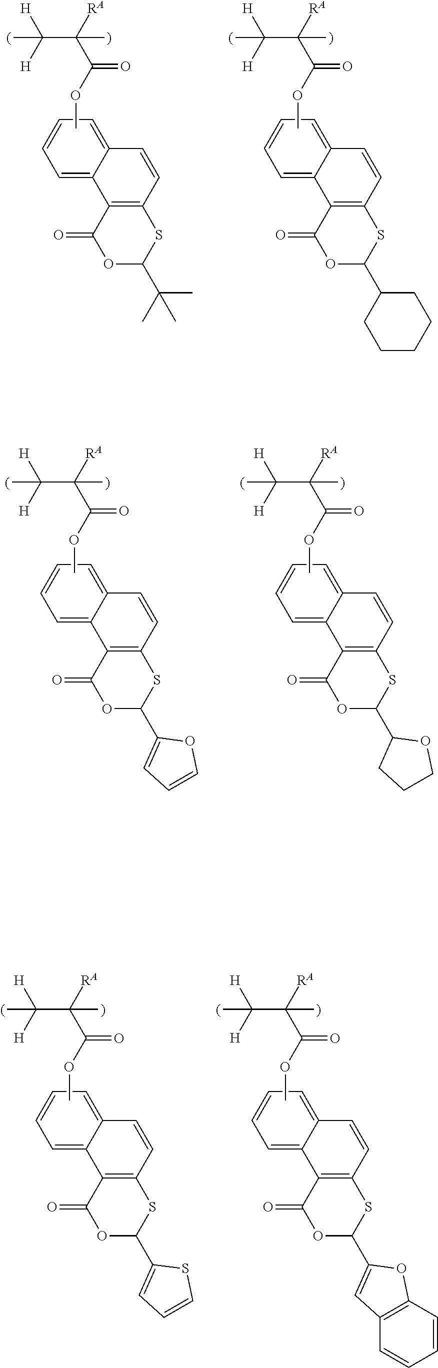

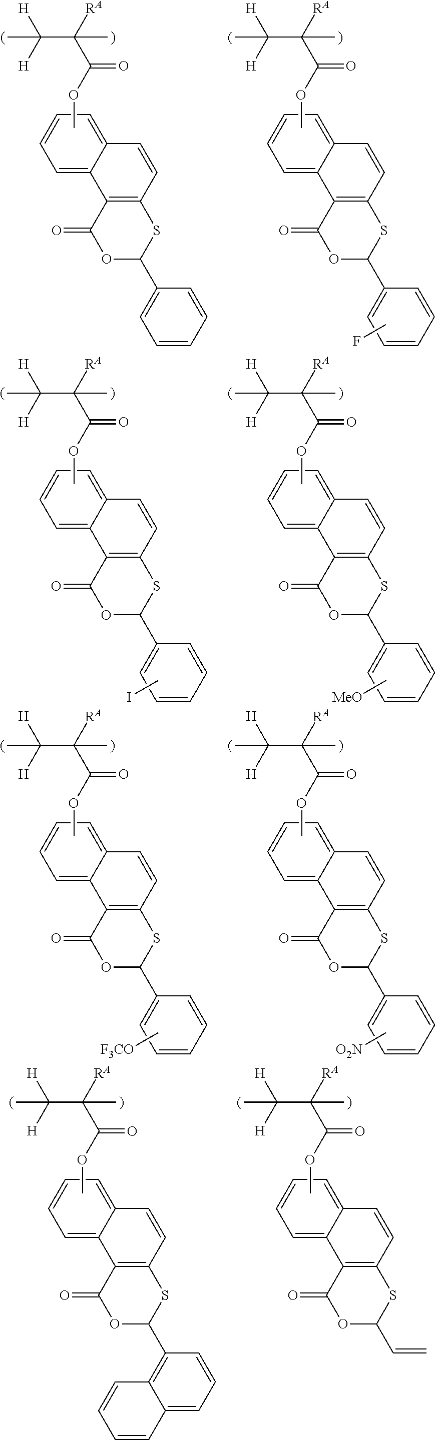

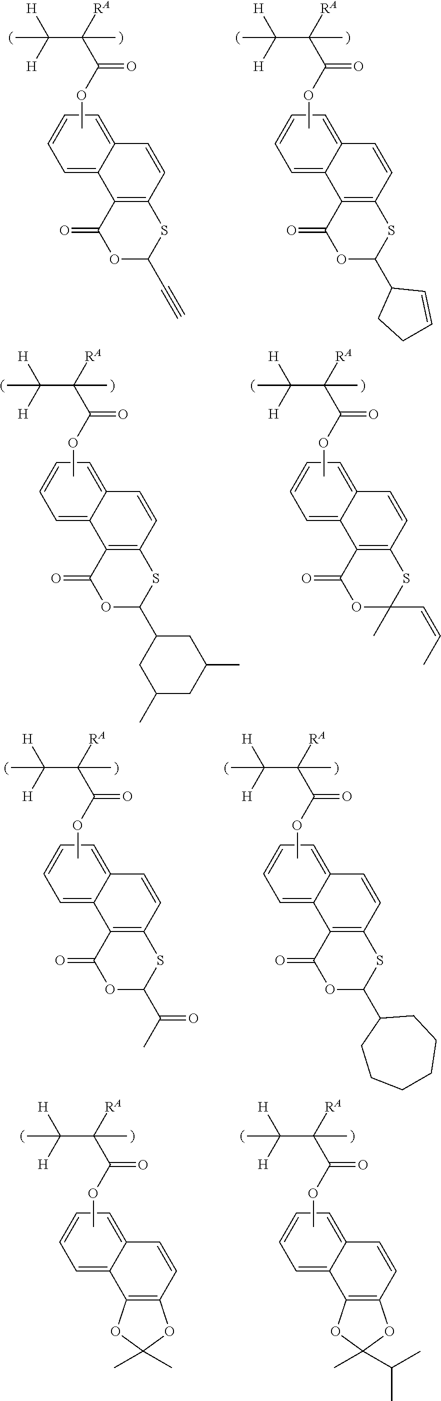

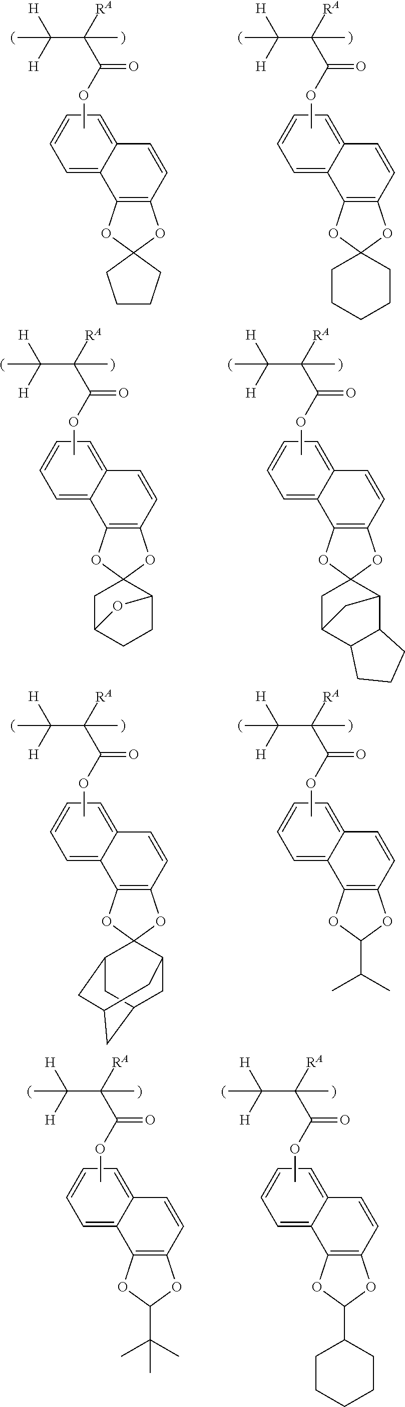

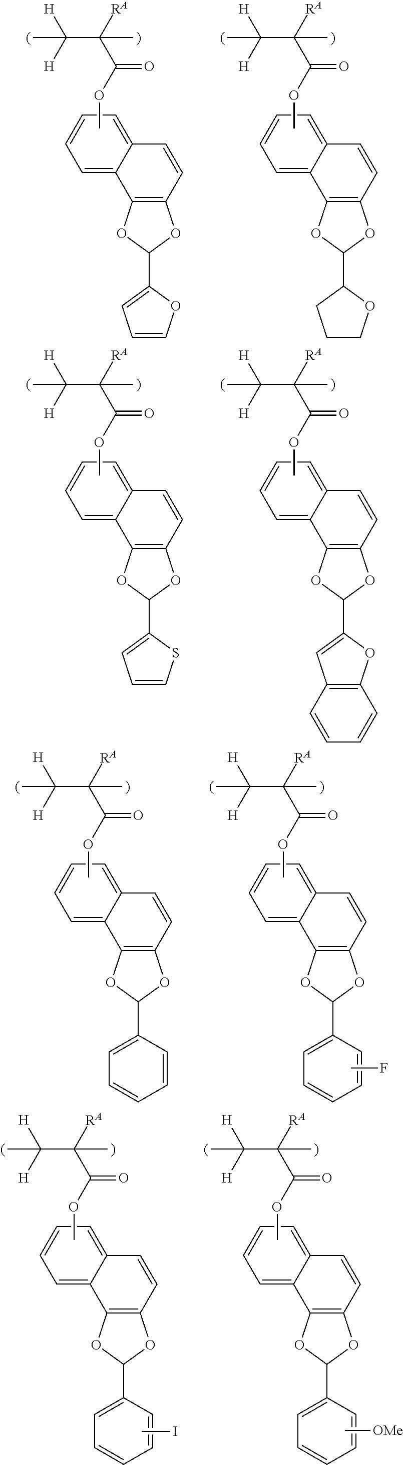

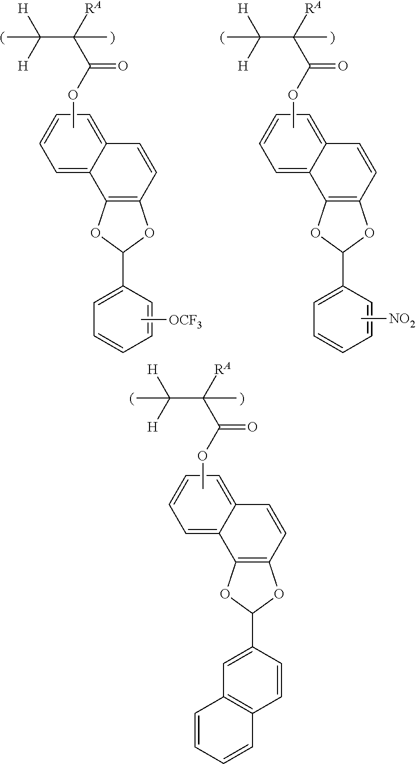

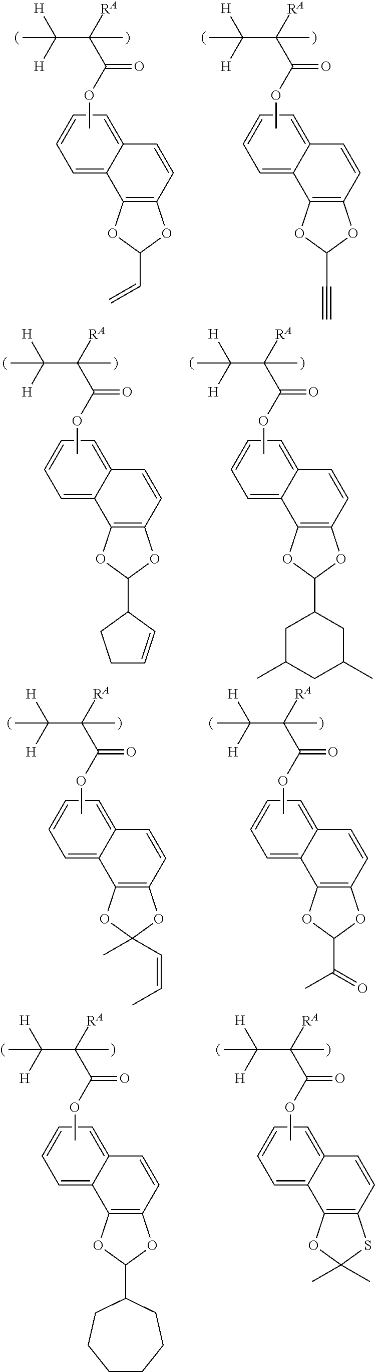

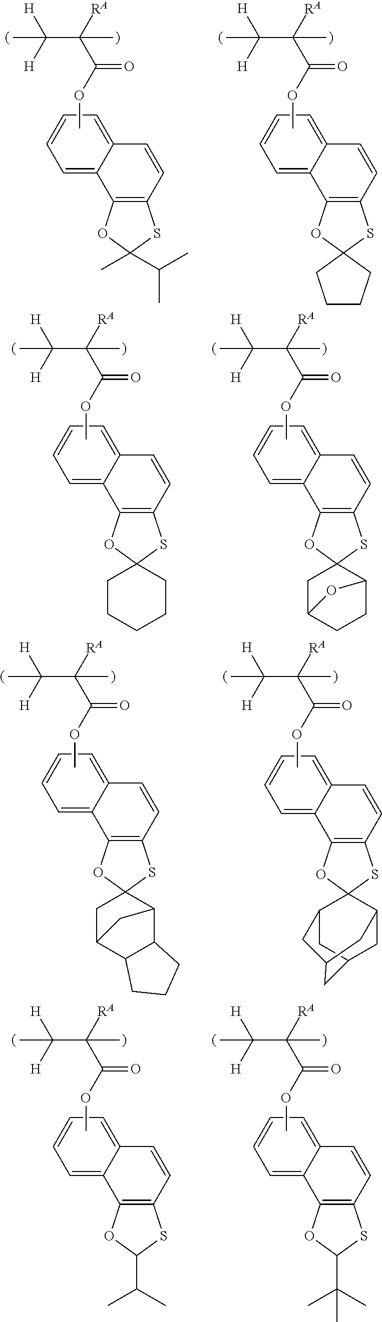

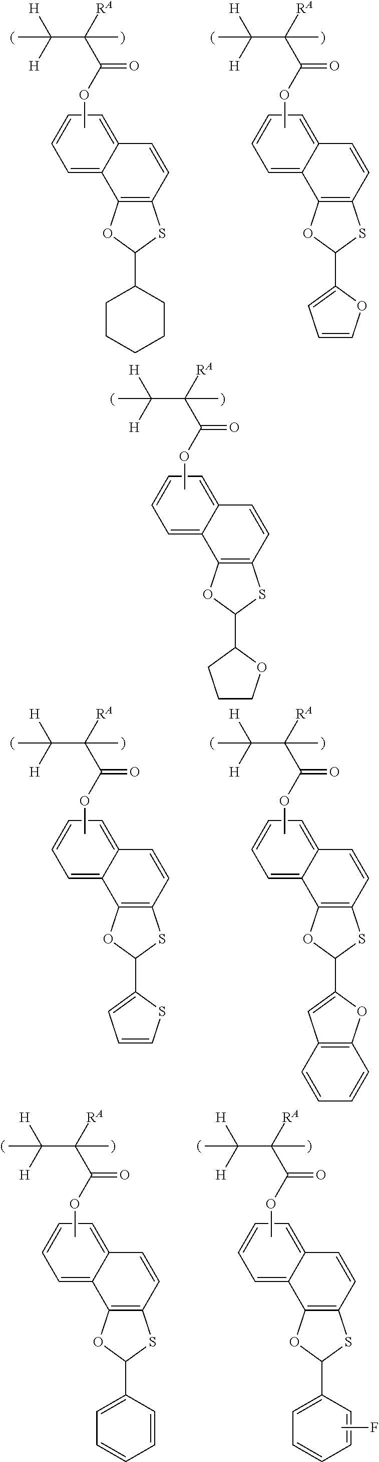

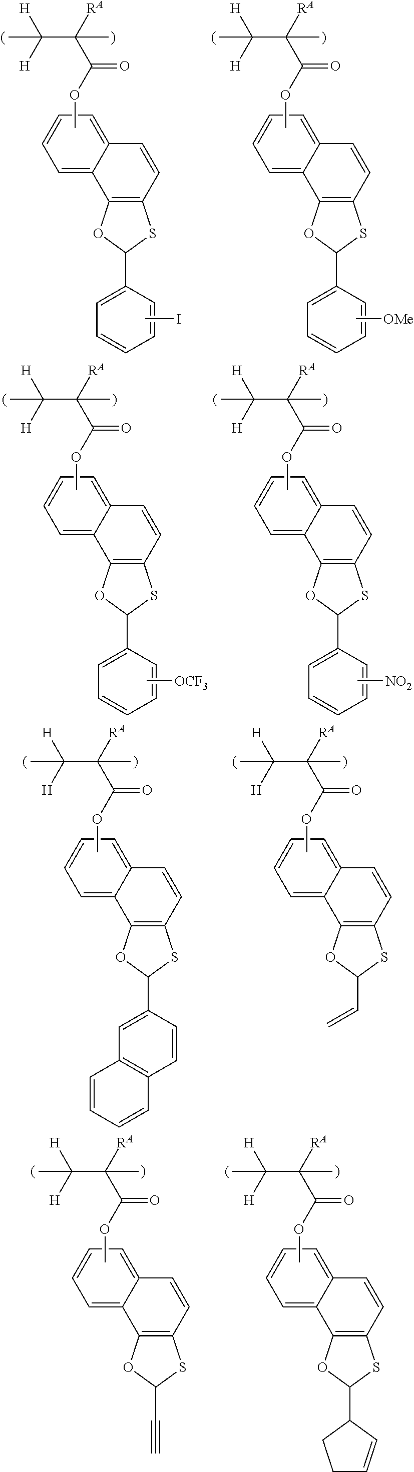

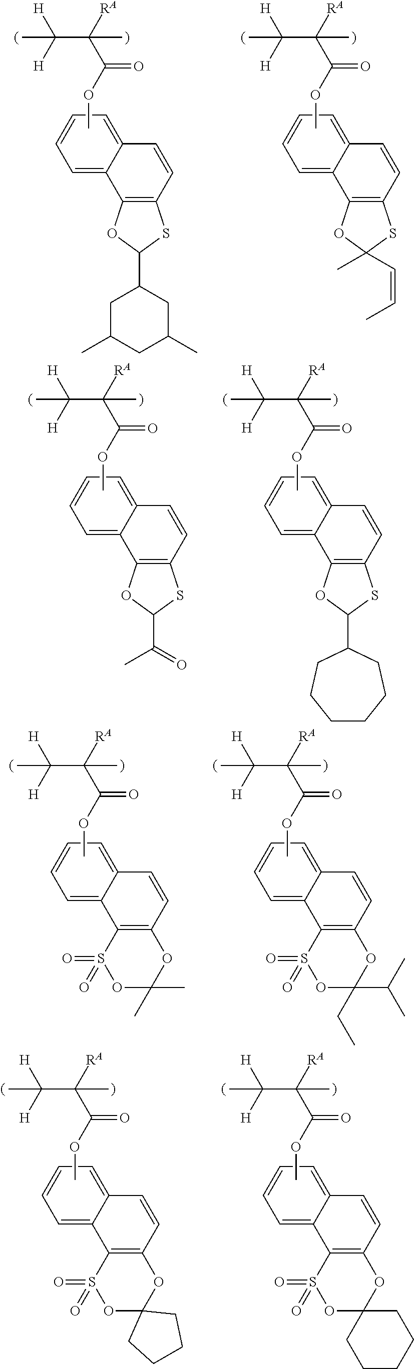

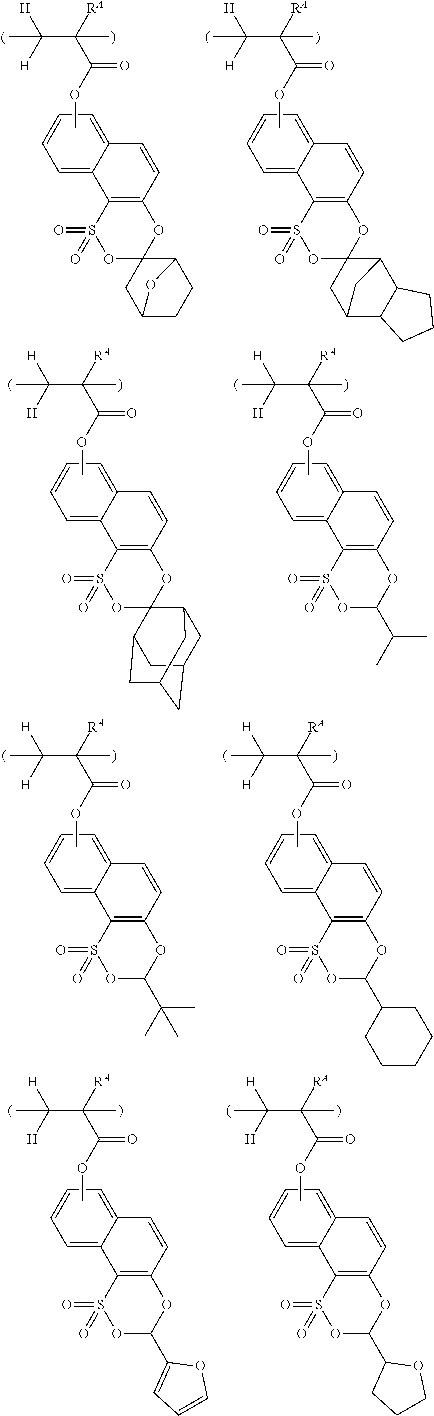

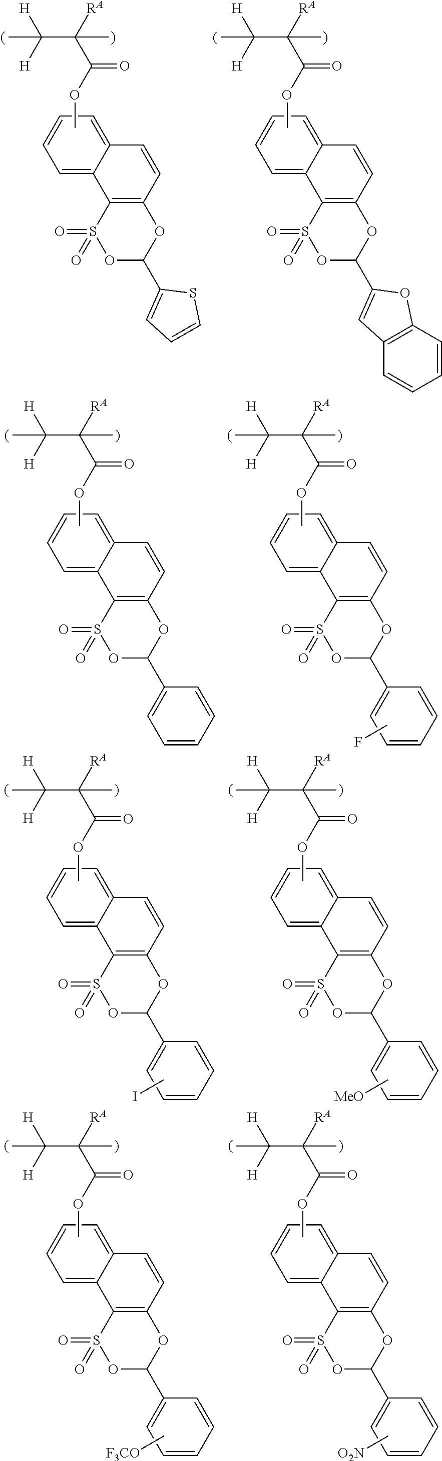

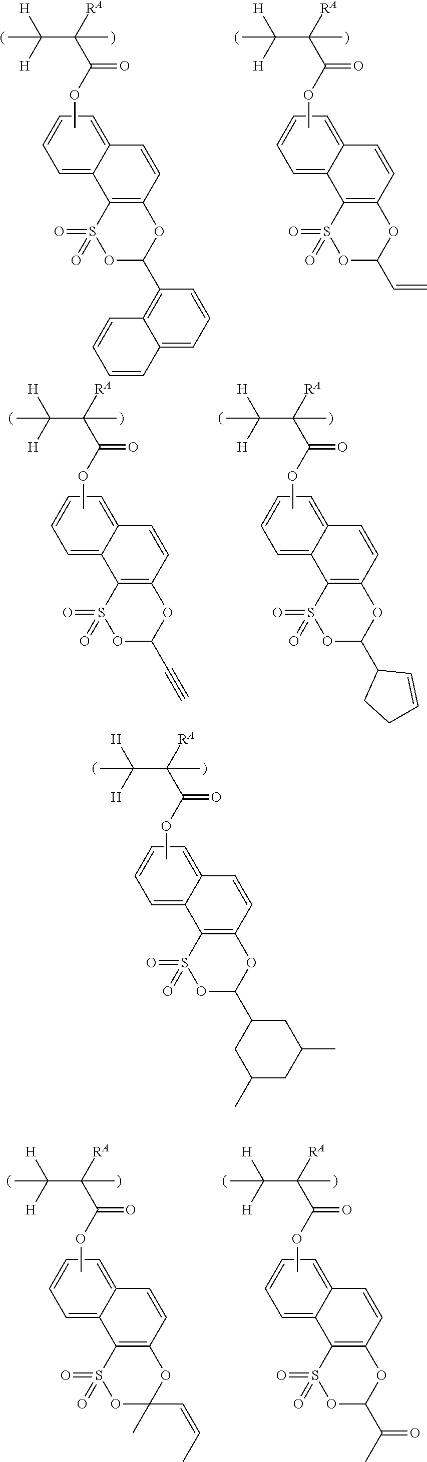

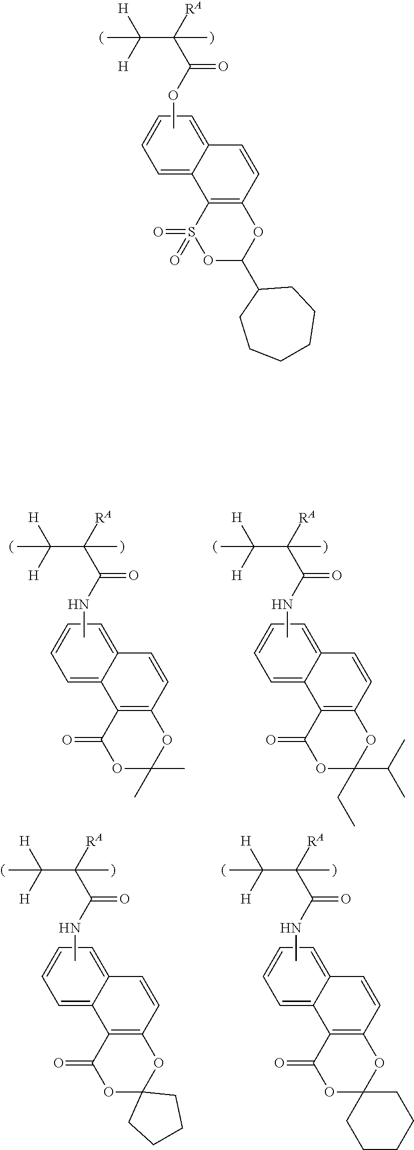

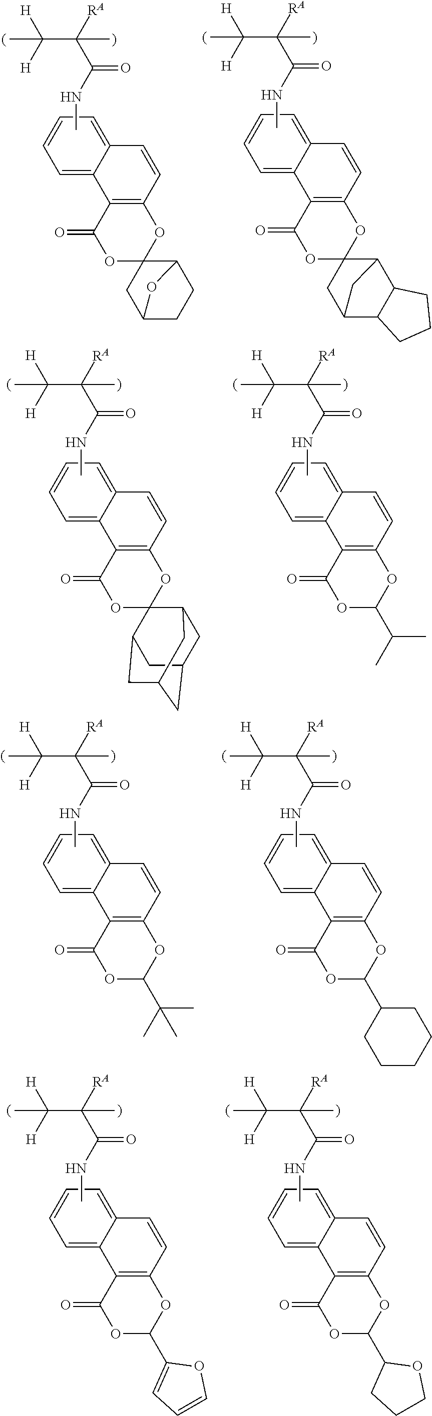

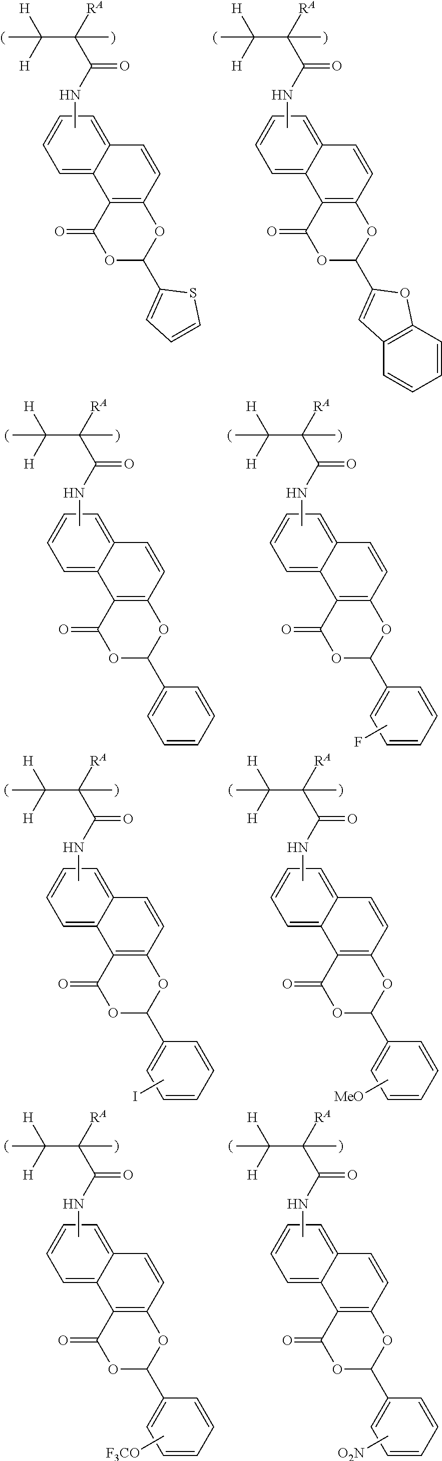

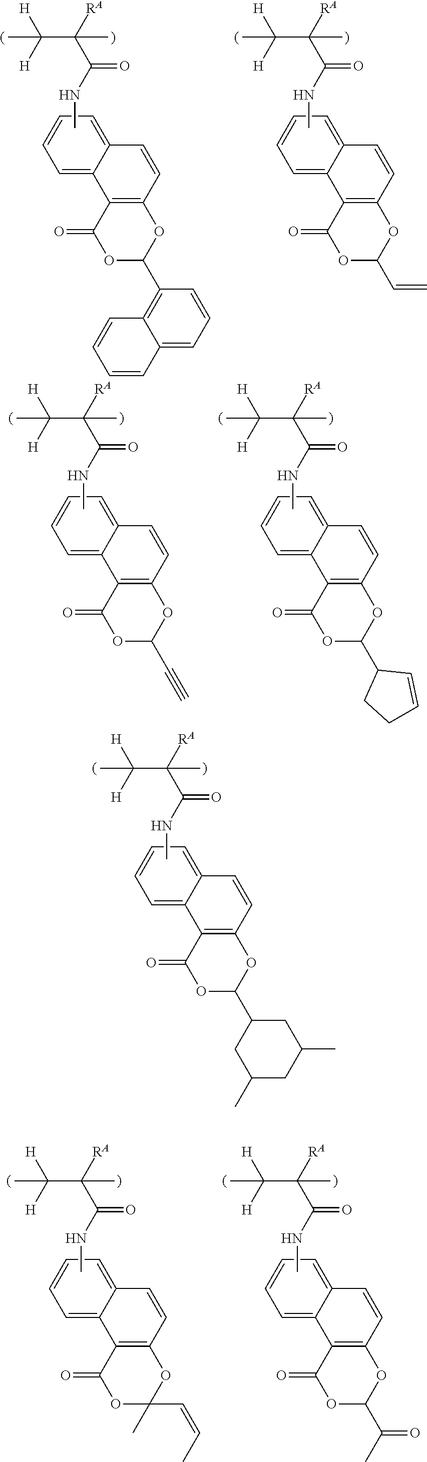

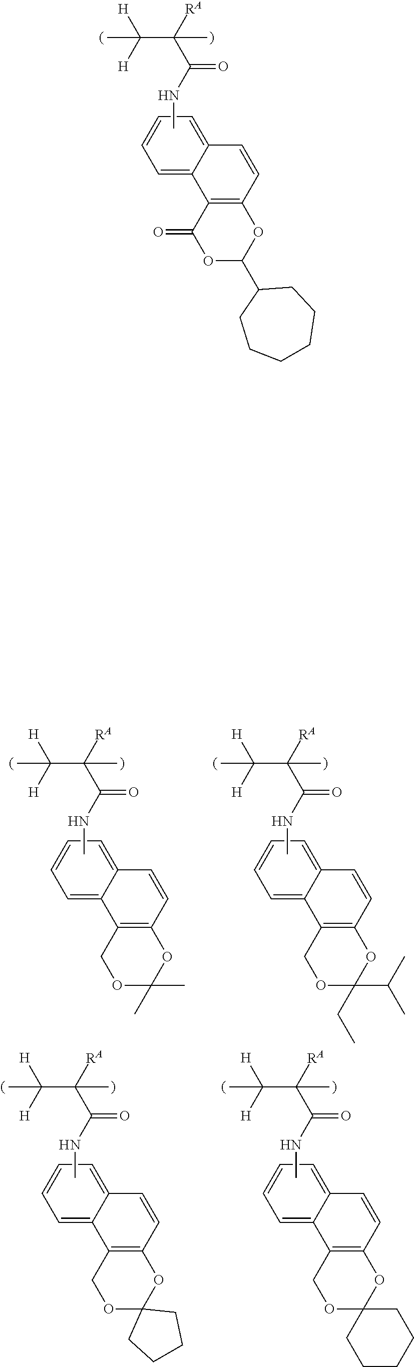

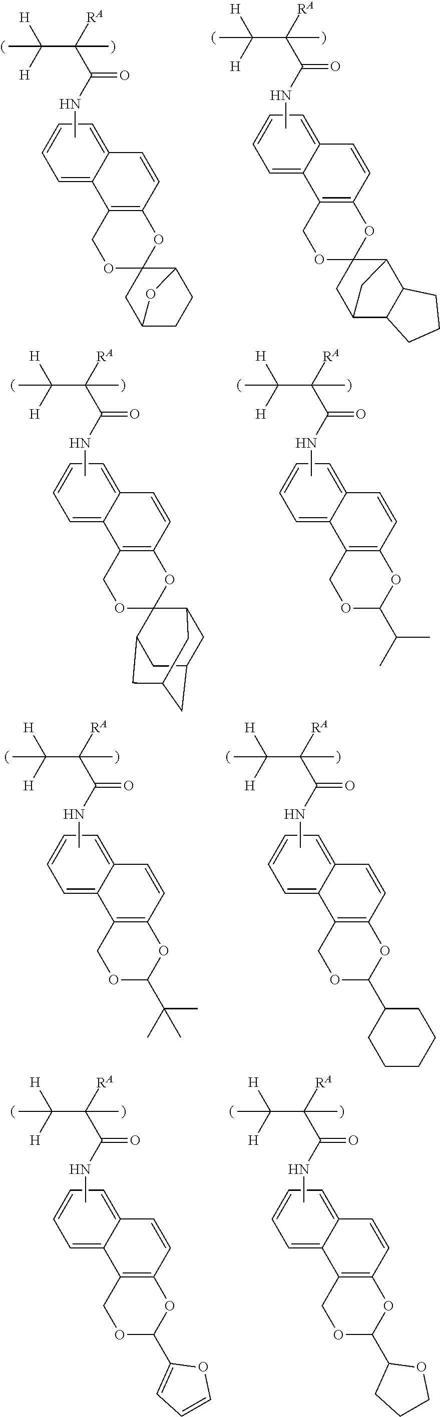

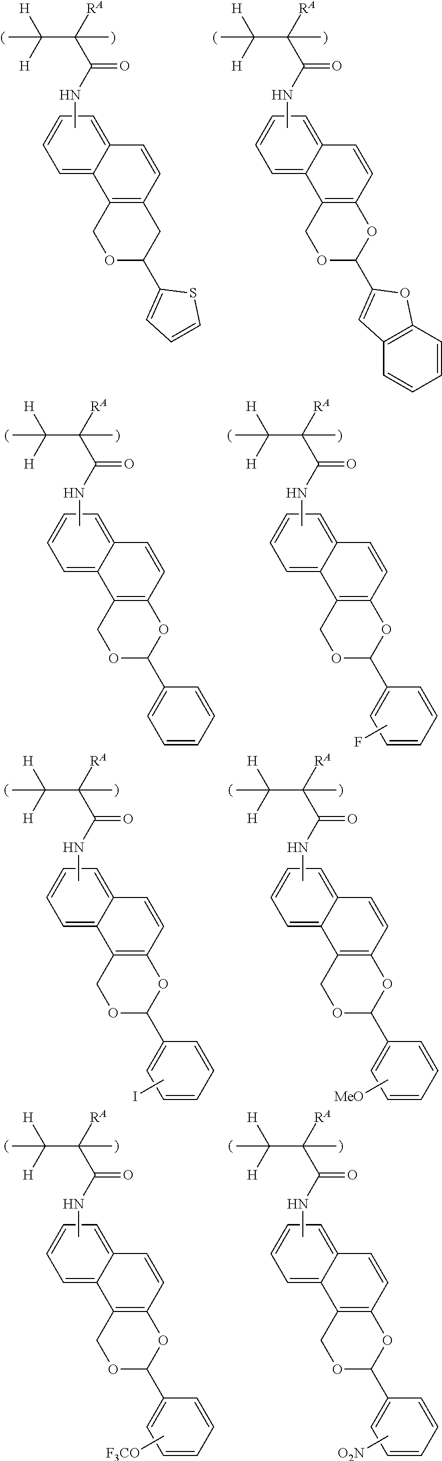

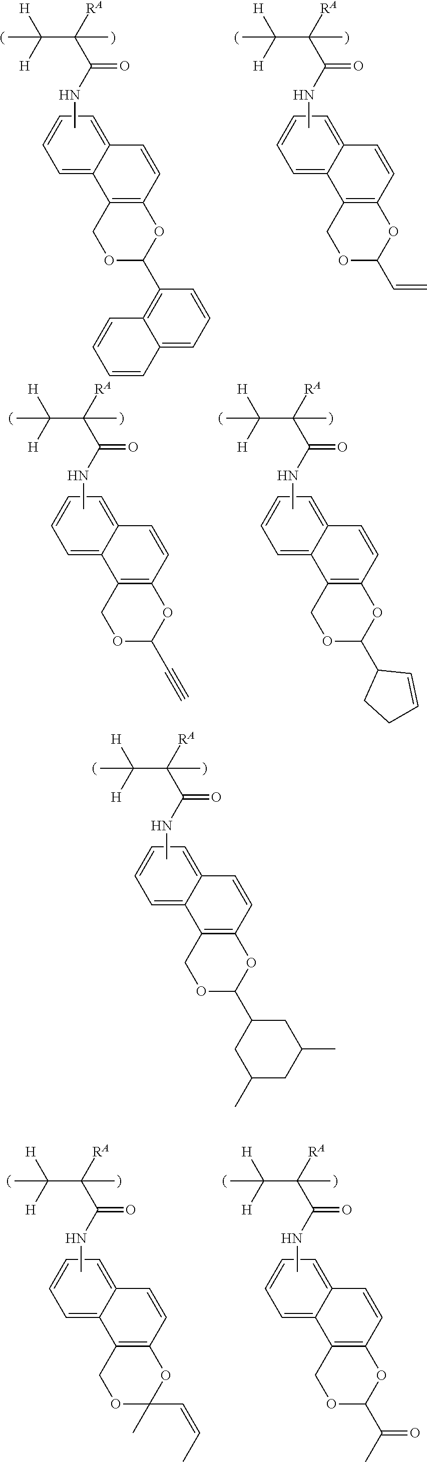

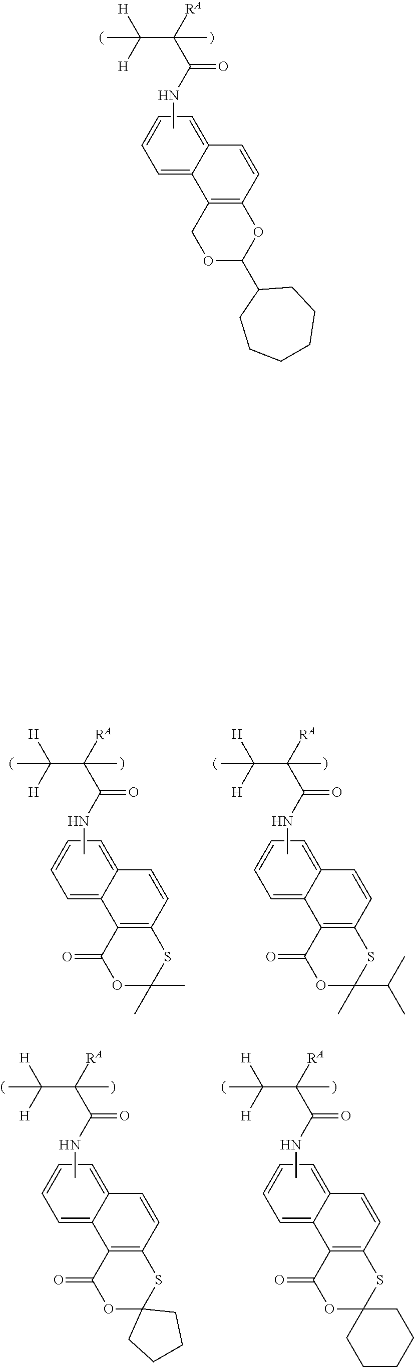

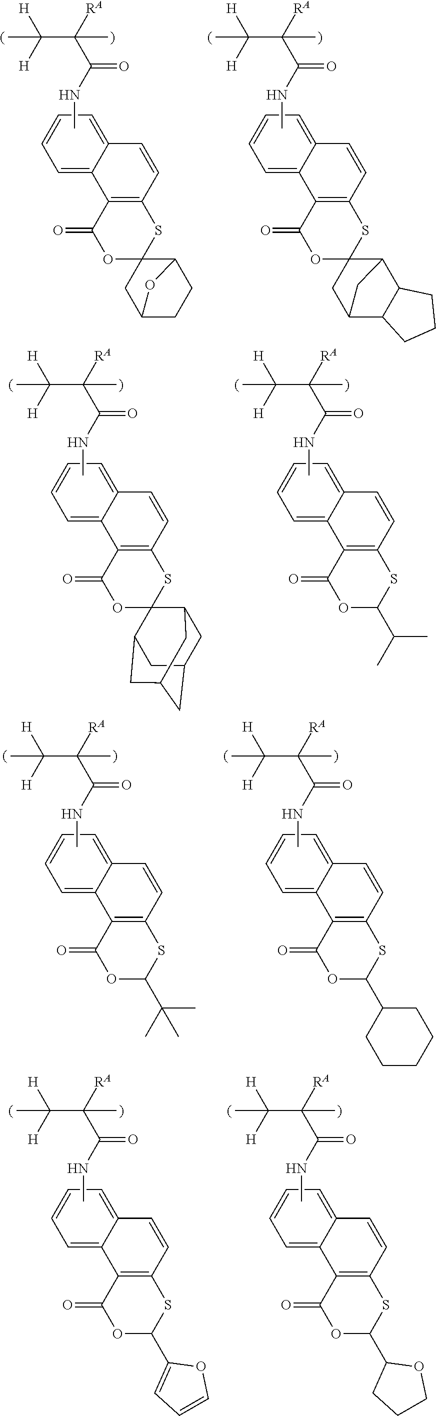

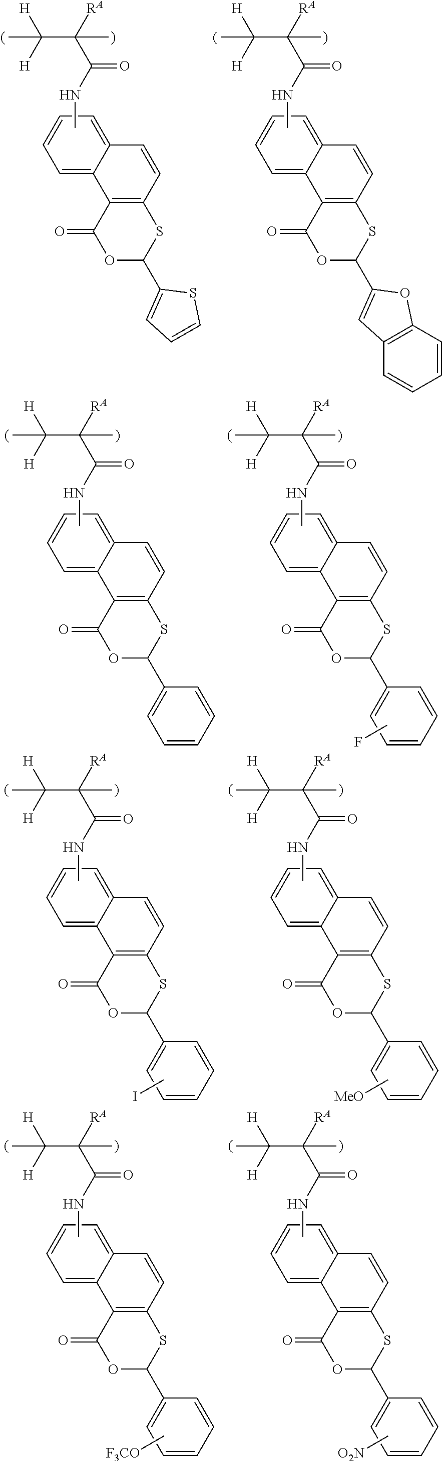

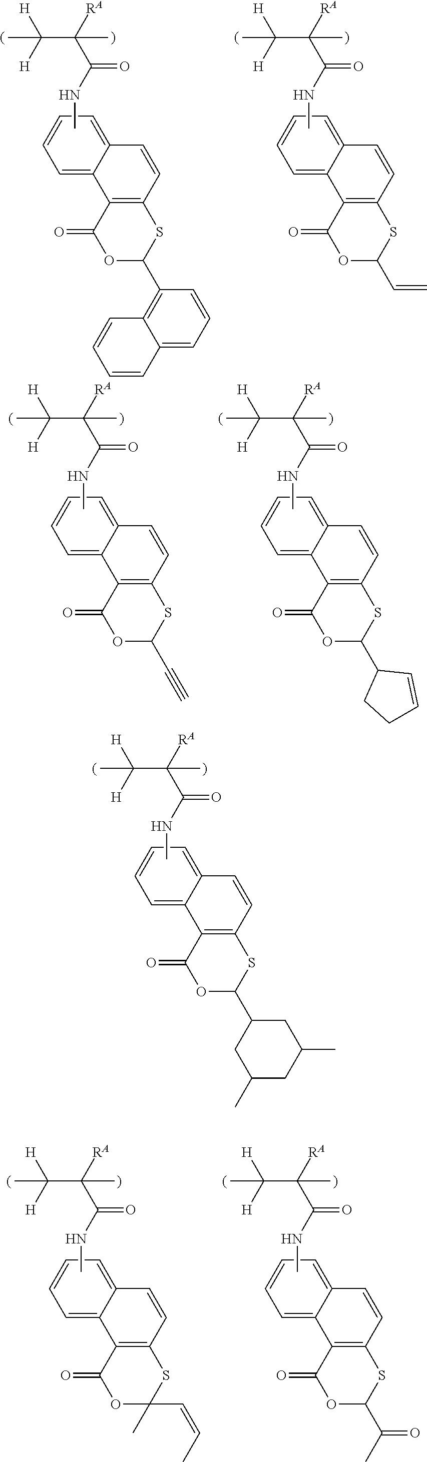

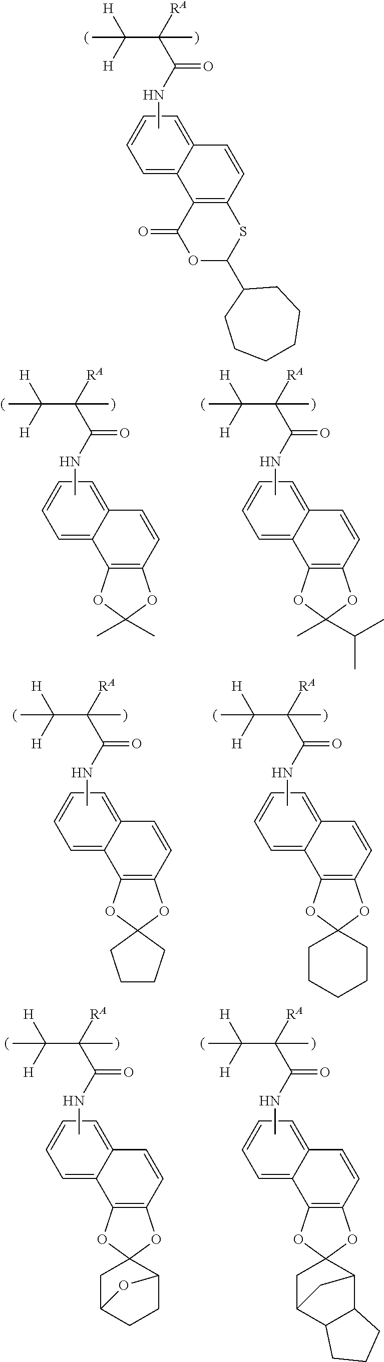

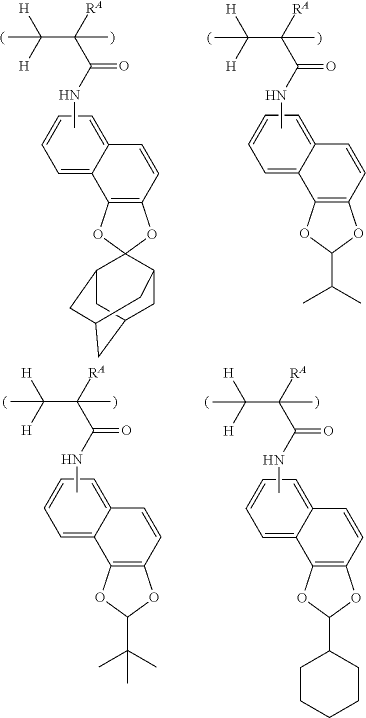

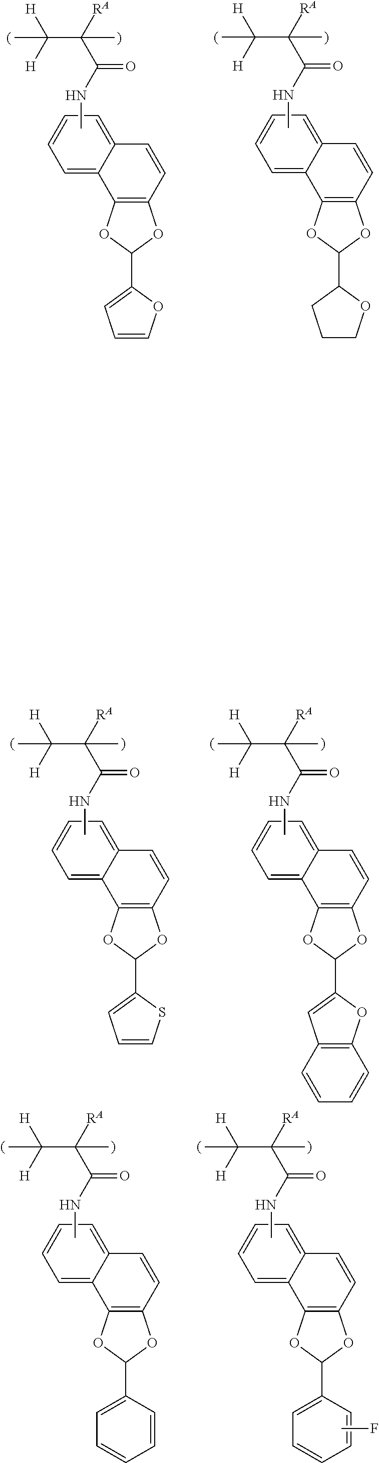

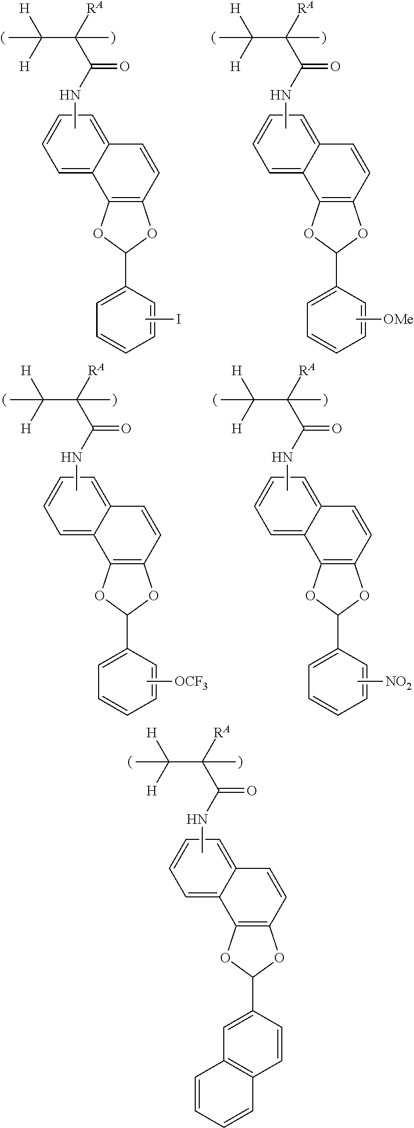

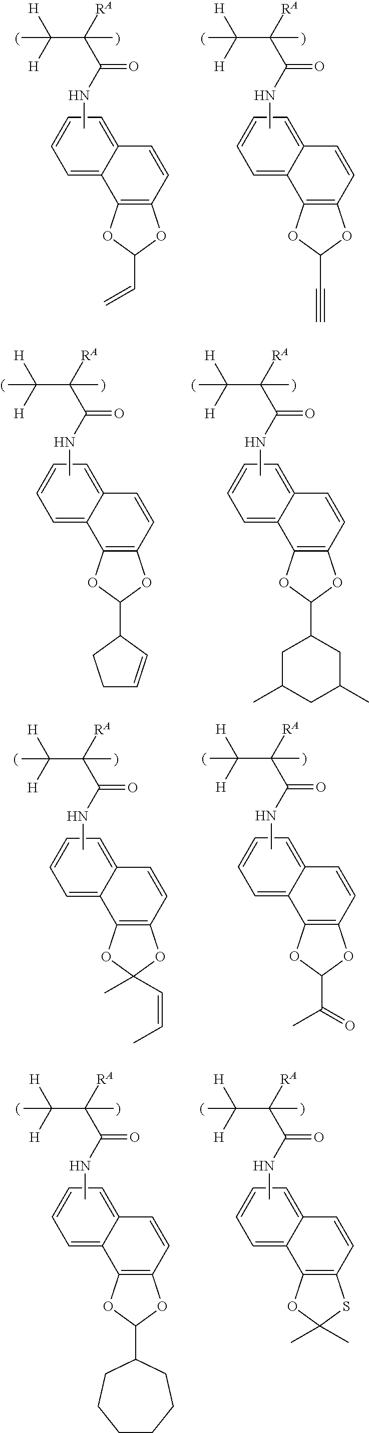

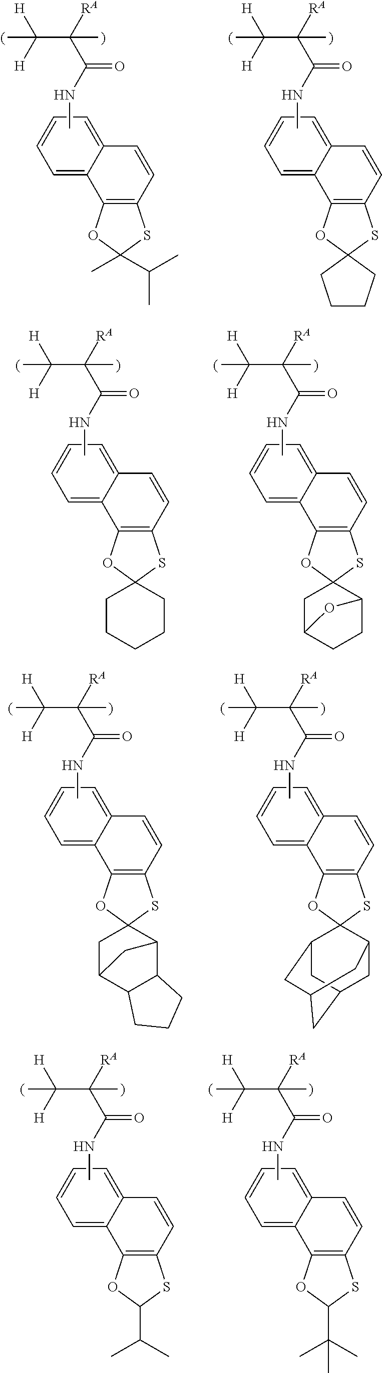

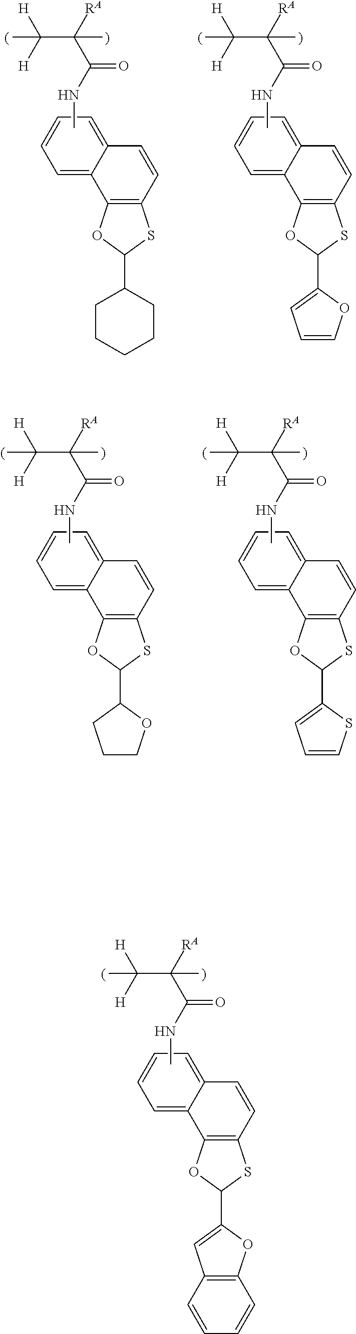

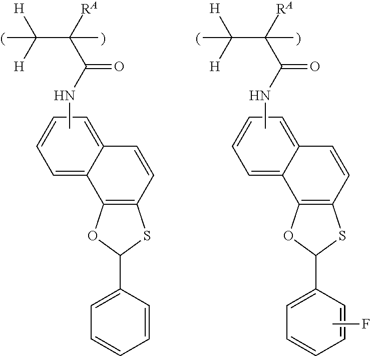

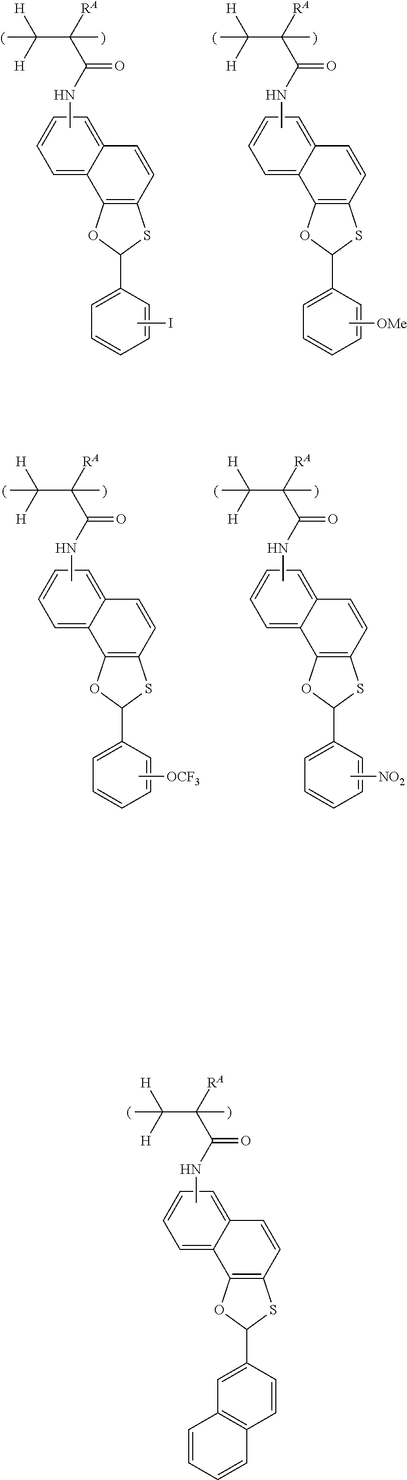

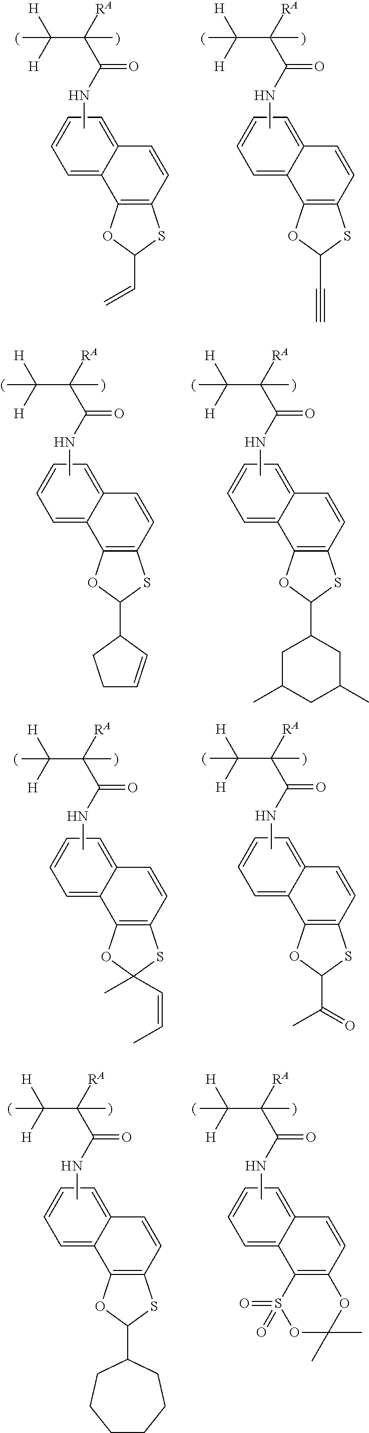

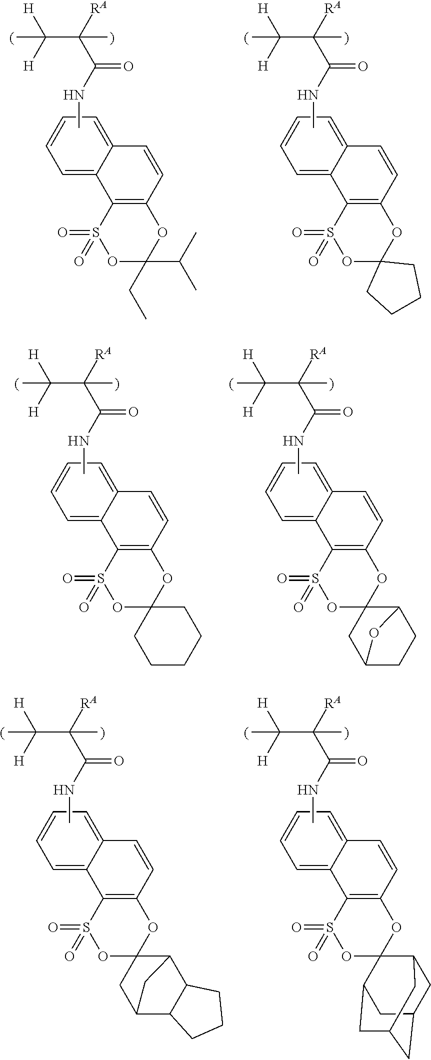

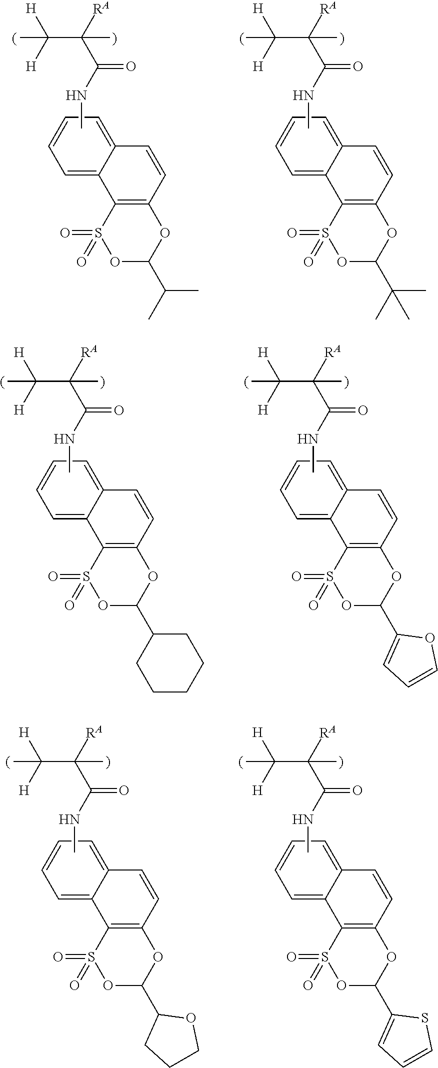

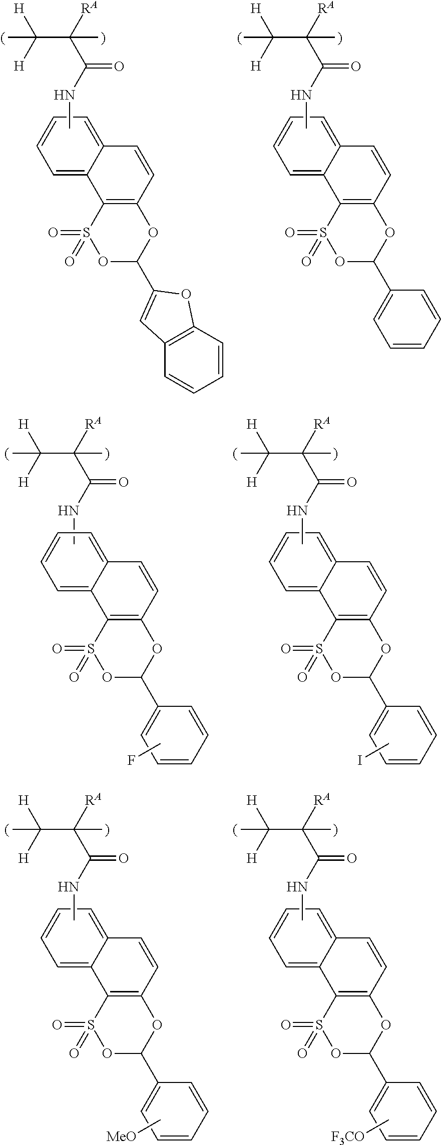

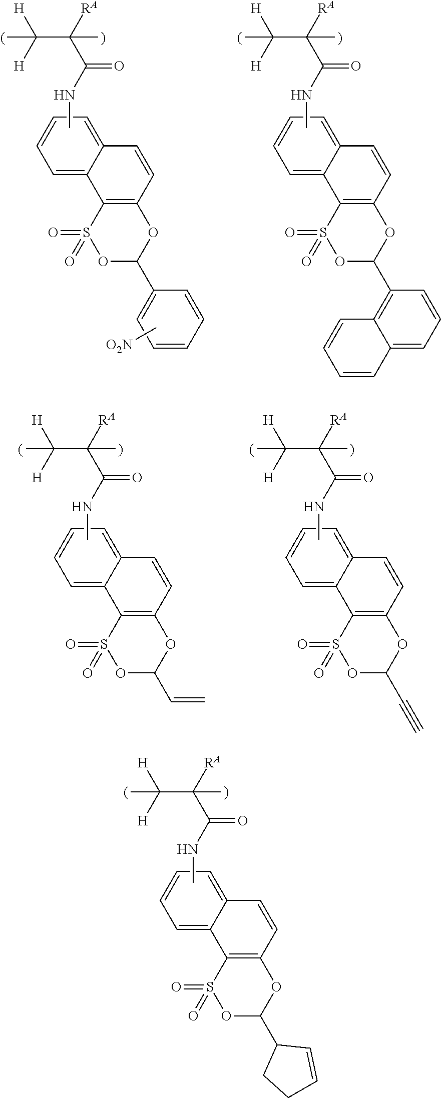

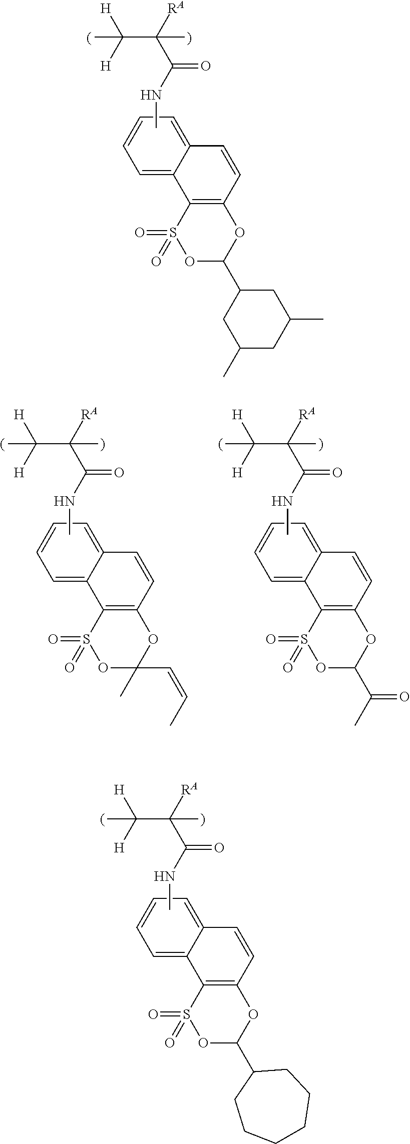

In a preferred embodiment, the polymer further comprises repeat units having the formula (b1) or repeat units having the formula (b2), referred to as repeat units (b1) or (b2), hereinafter.

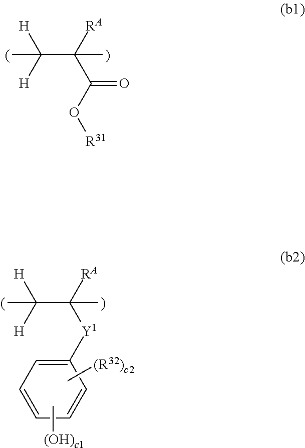

In formulae (b1) and (b2), R^{A} is each independently hydrogen, fluorine, methyl or trifluoromethyl. Y¹ is a single bond or *—C(═O)—O— wherein * designates a point of attachment to the carbon atom in the backbone. R³¹ is hydrogen or a C₁-C₂₀ group which contains at least one structure selected from hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—). R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. A plurality of R³² may be identical or different when c2 is 2, 3 or 4. The subscript c1 is 1, 2, 3 or 4, c2 is 0, 1, 2, 3 or 4, and 1≤c1+c2≤5.

Examples of the repeat unit (b1) are shown below, but not limited thereto. Herein R^{A} is as defined above.

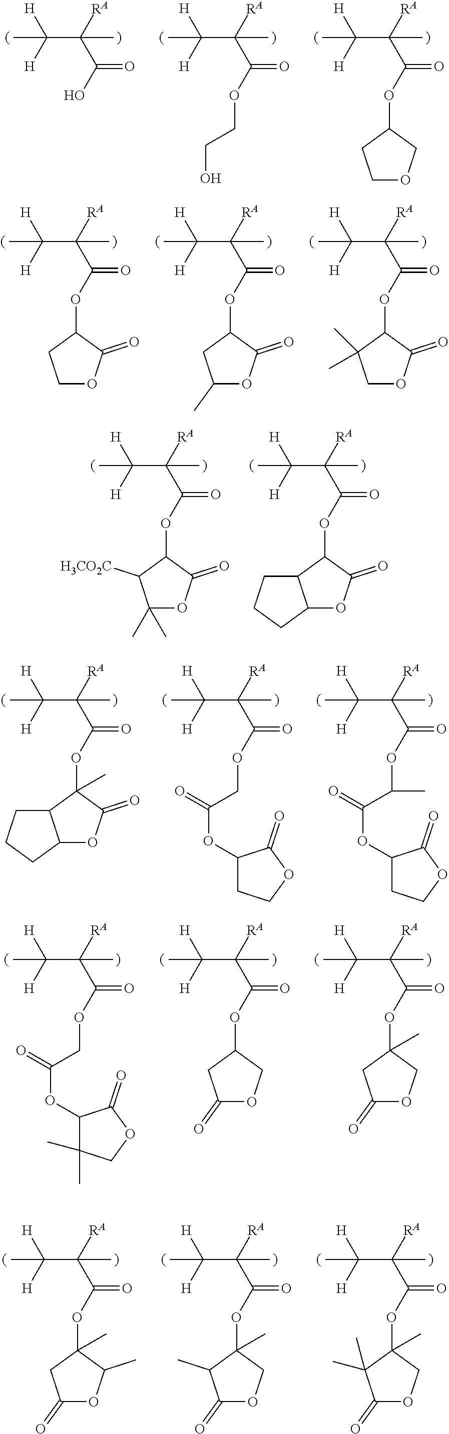

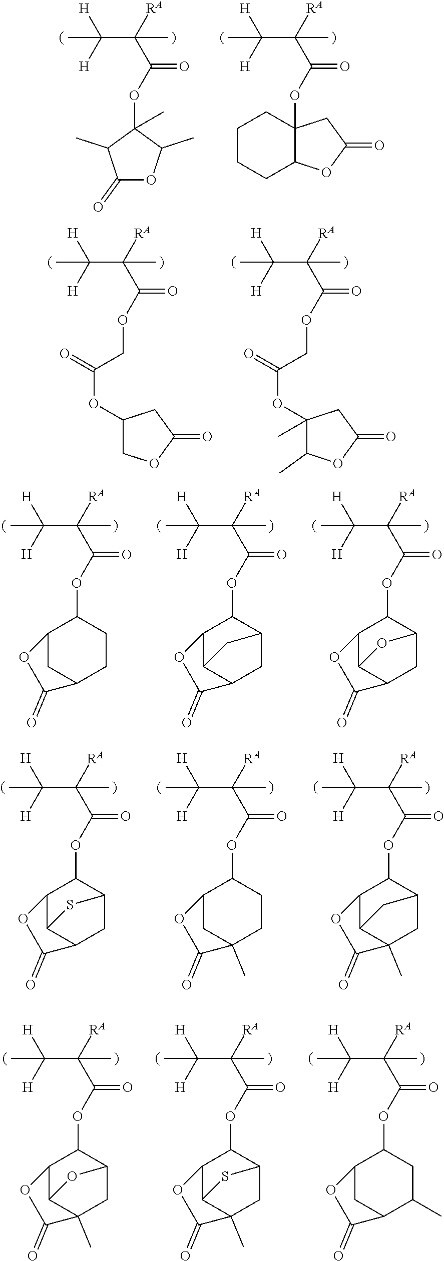

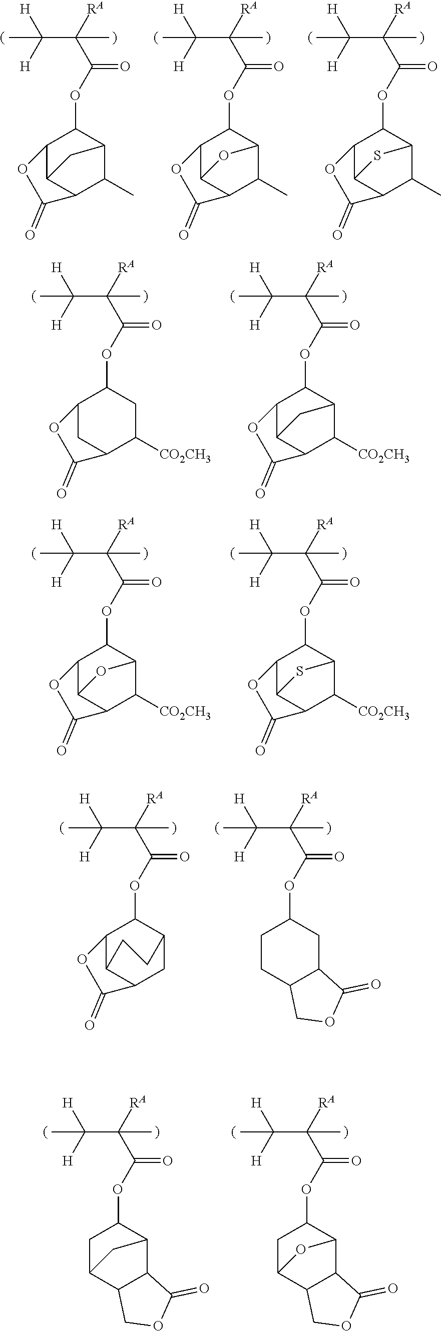

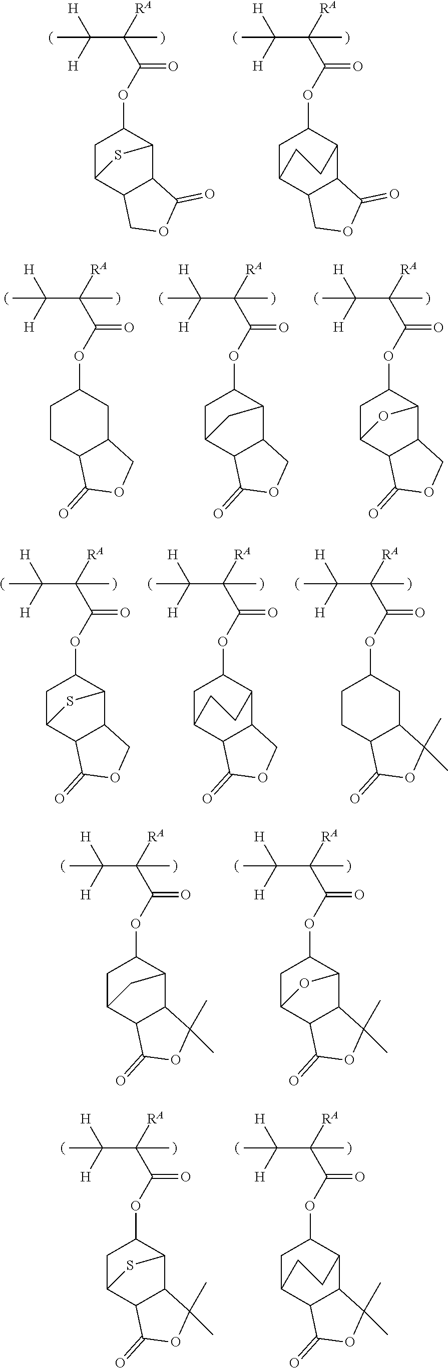

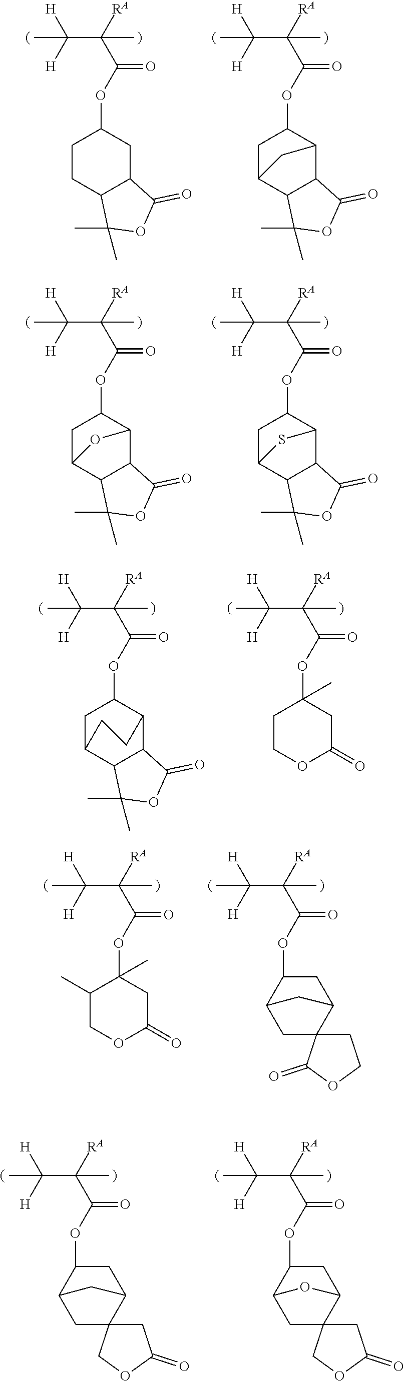

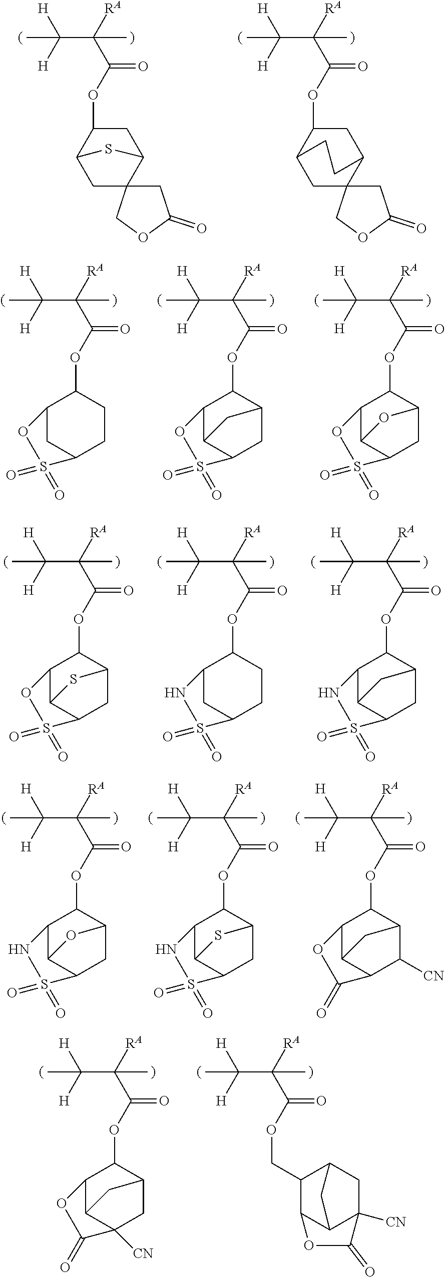

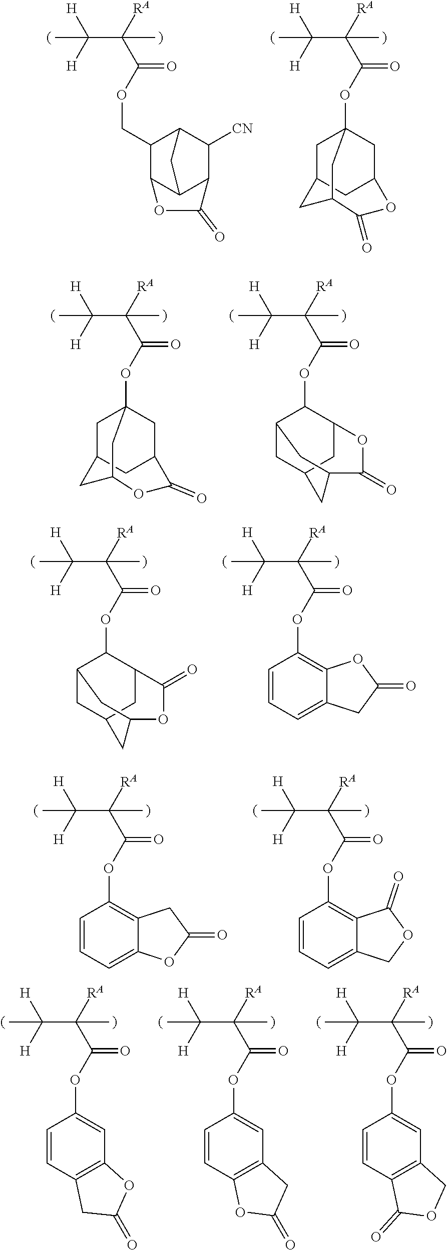

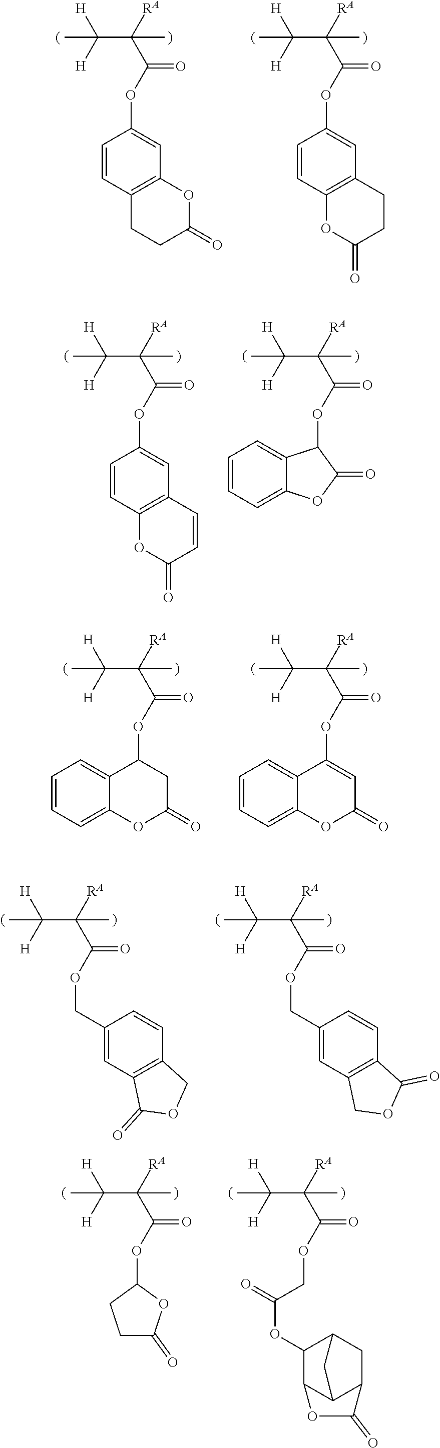

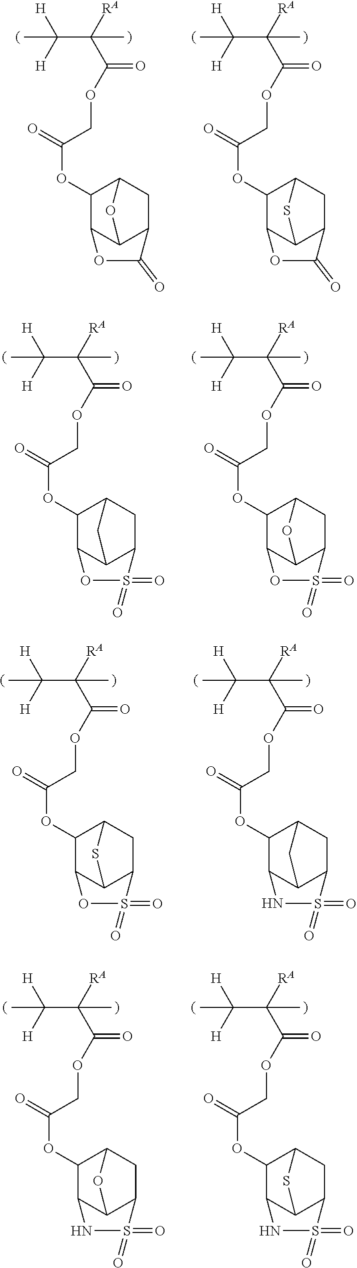

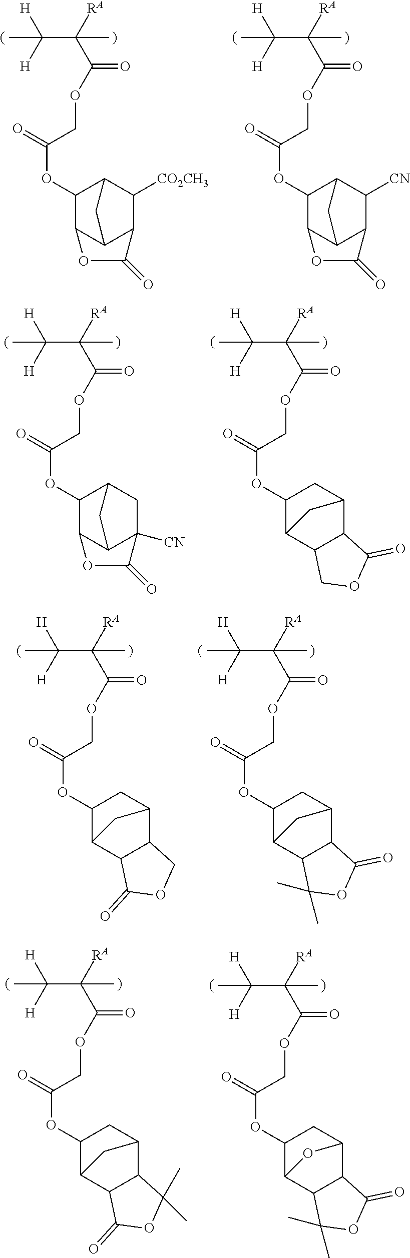

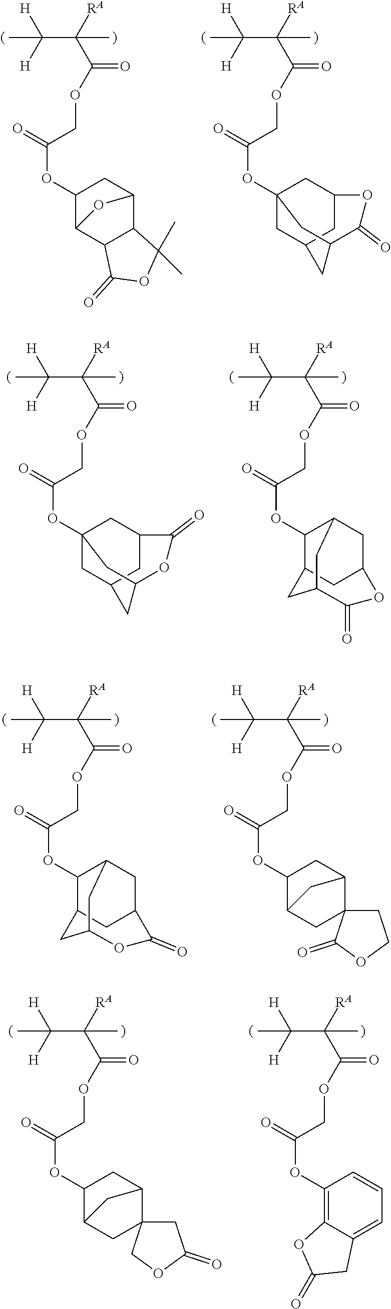

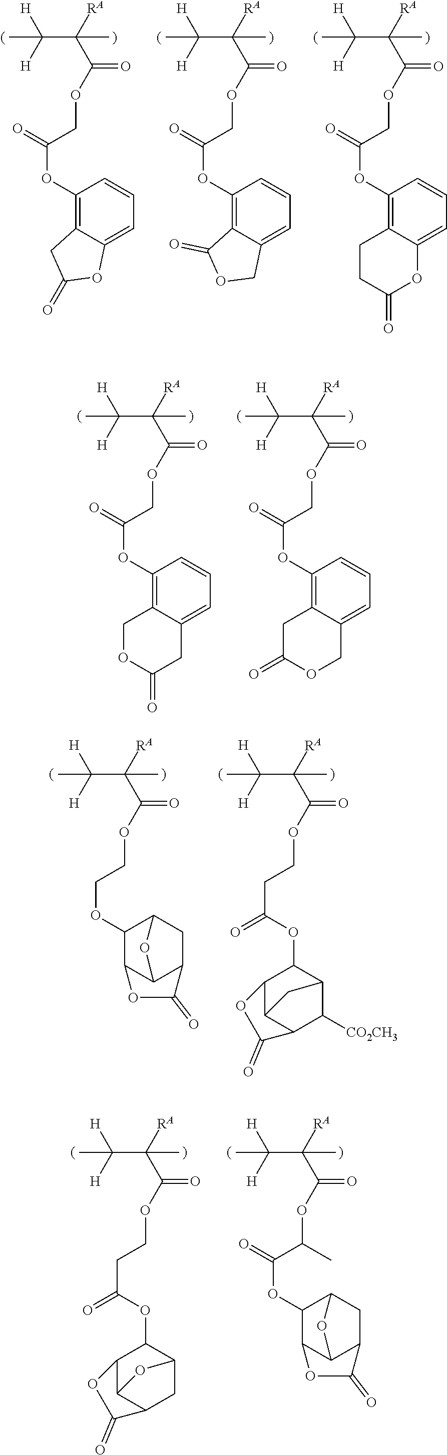

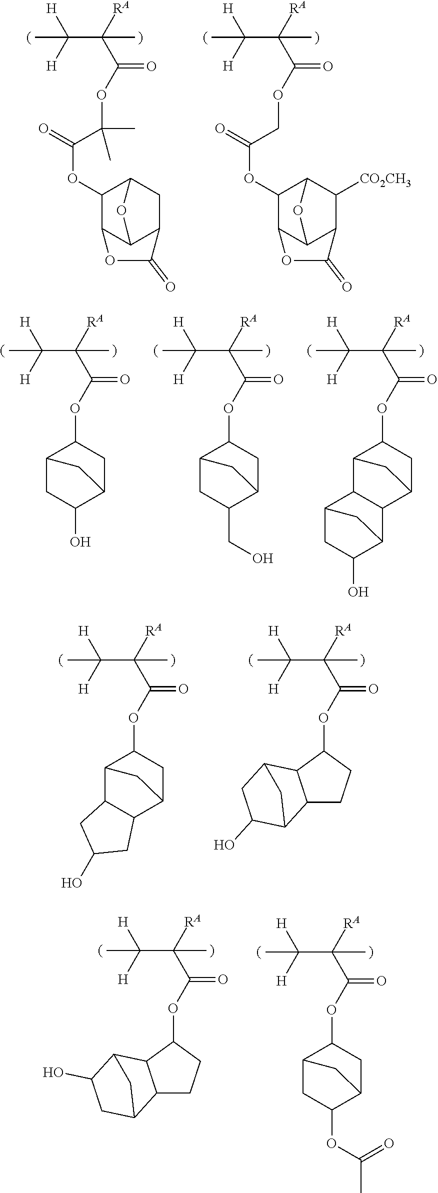

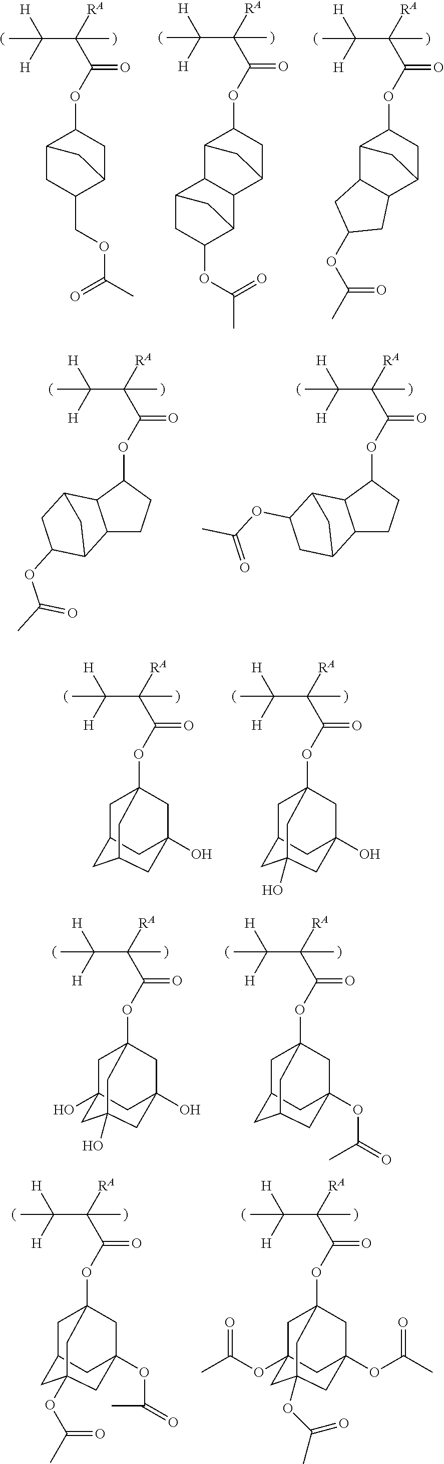

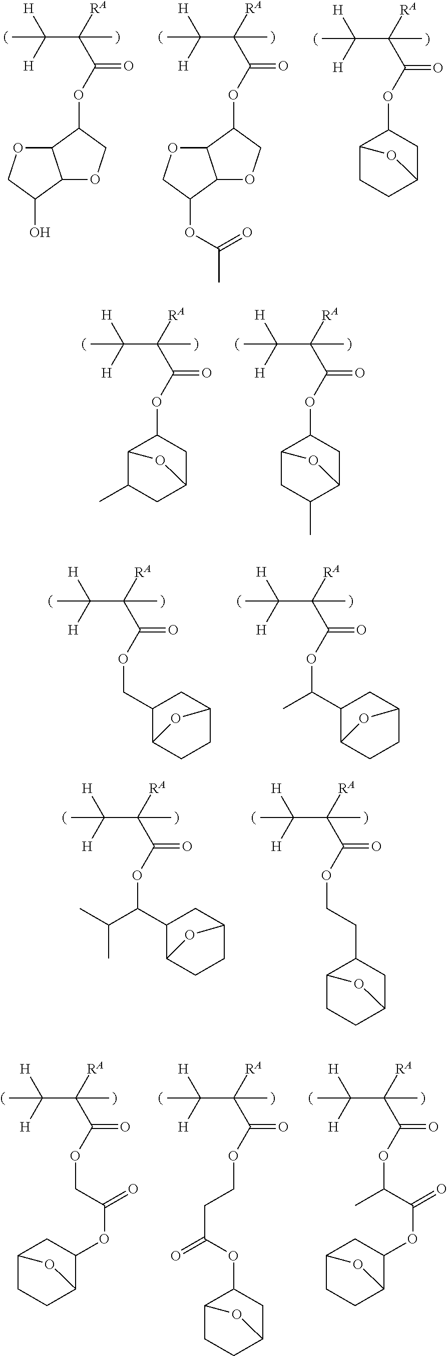

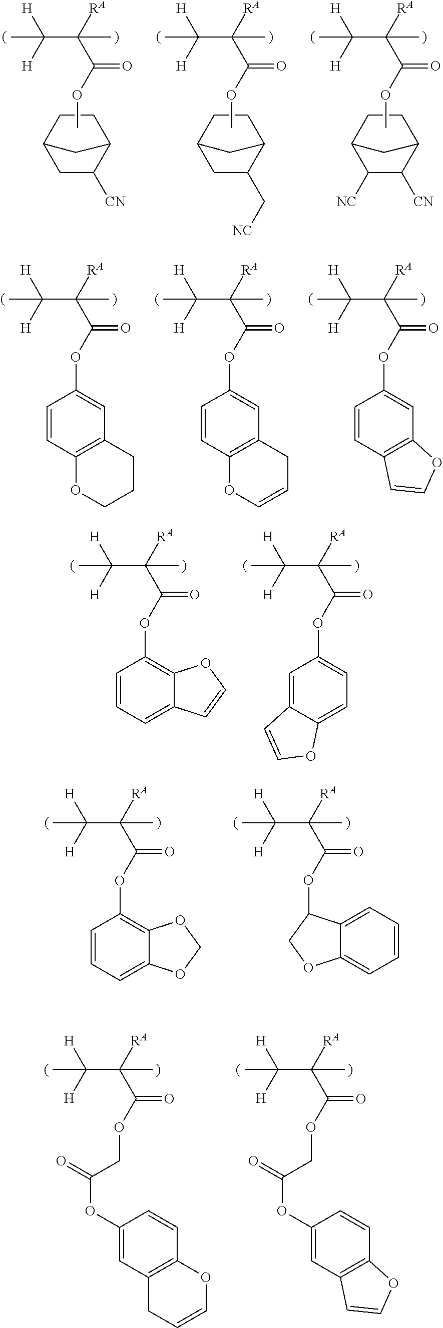

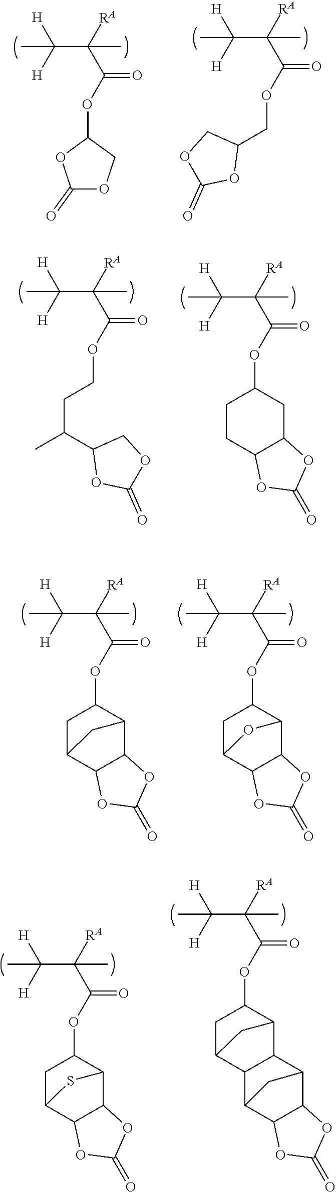

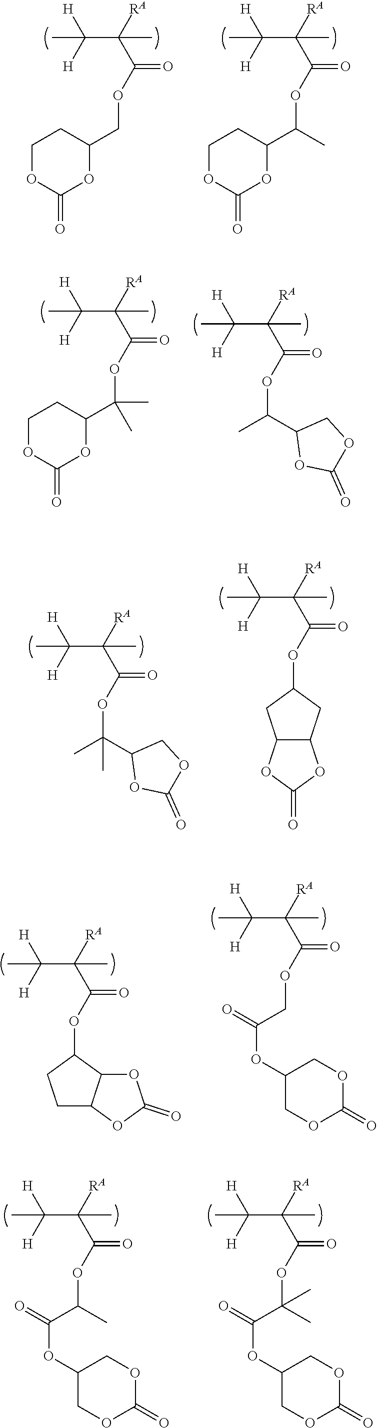

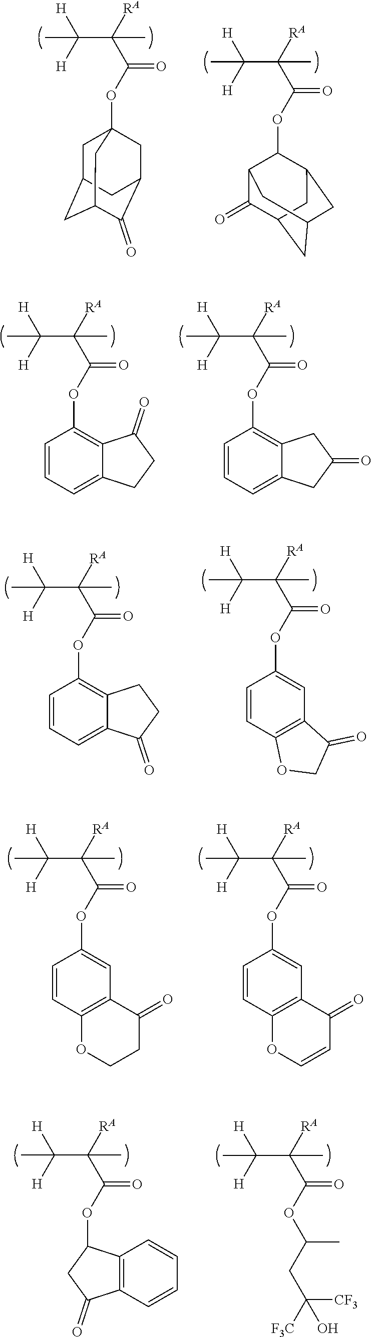

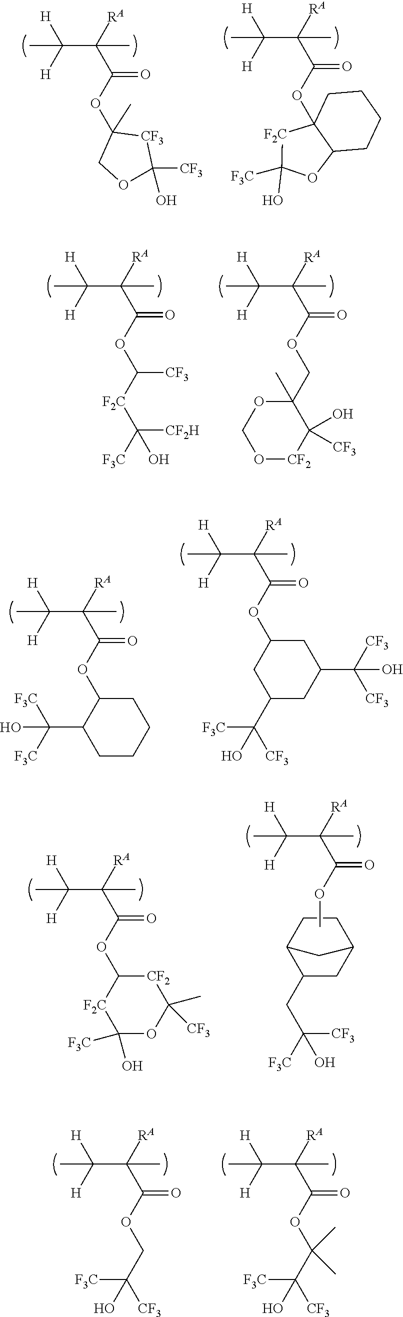

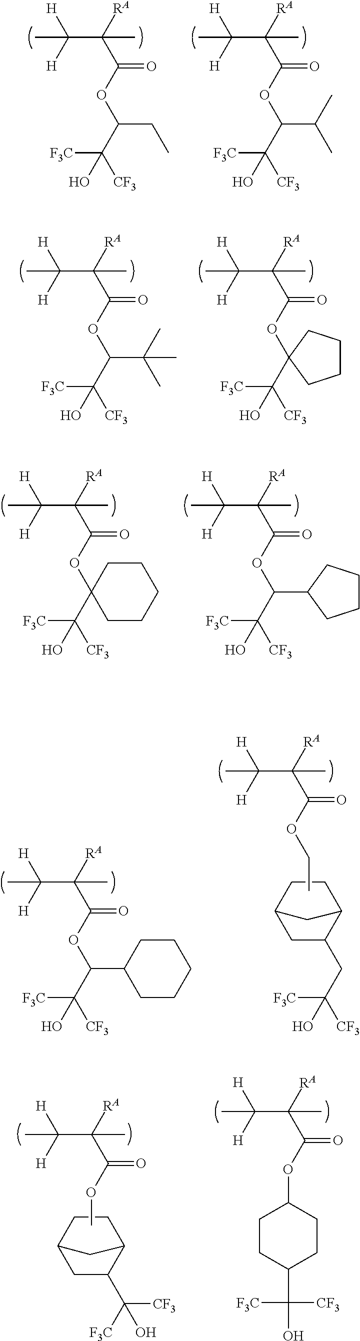

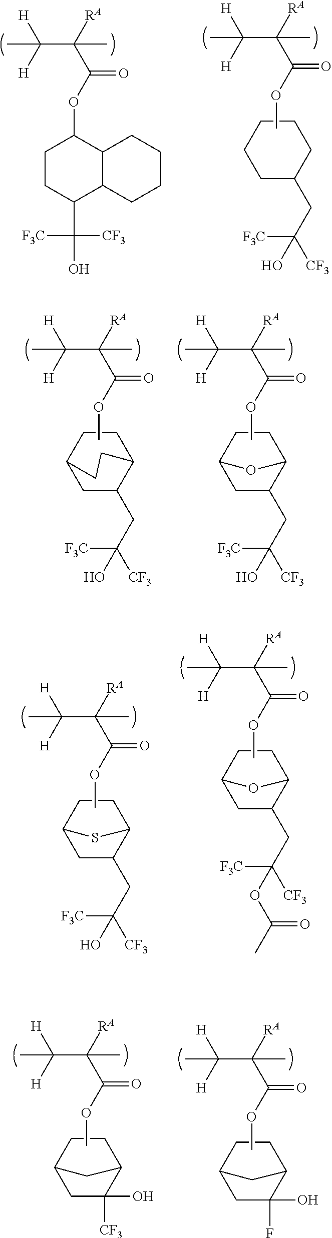

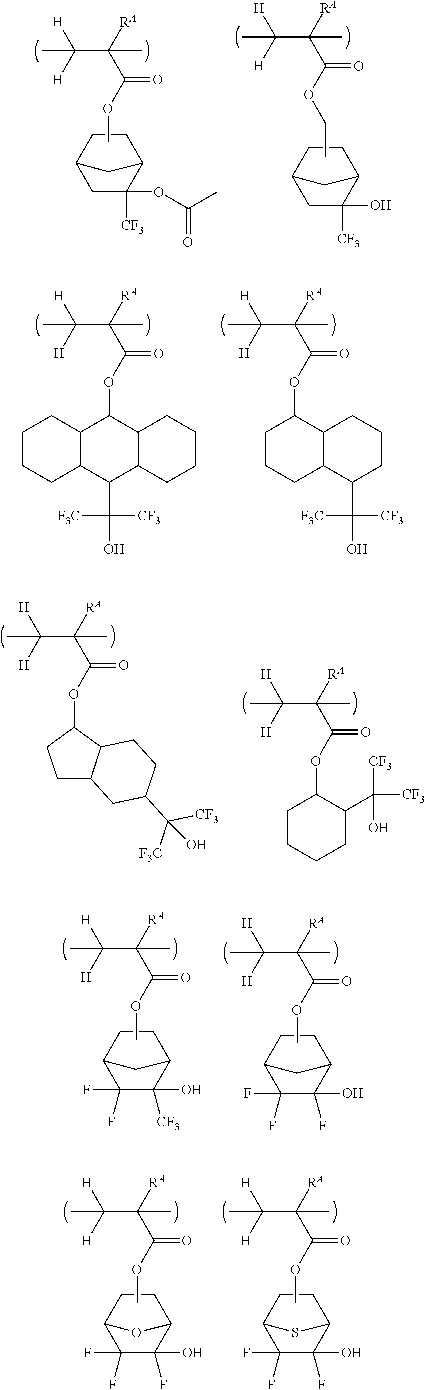

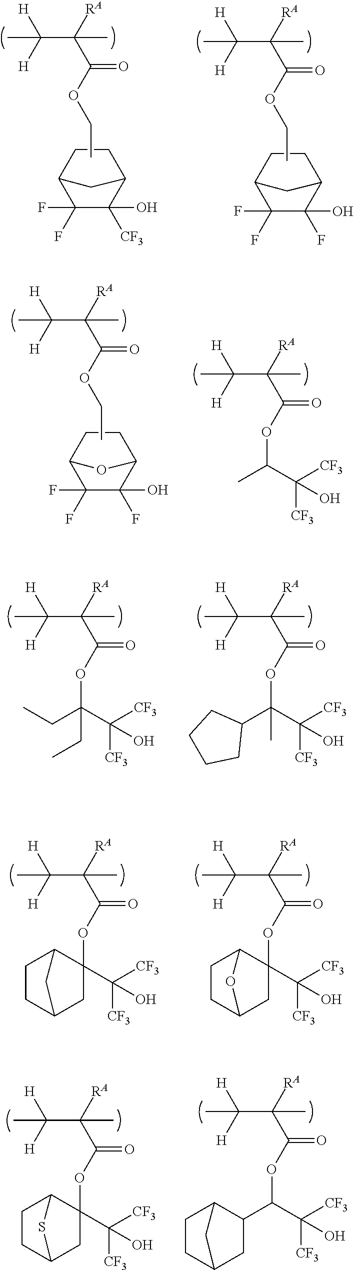

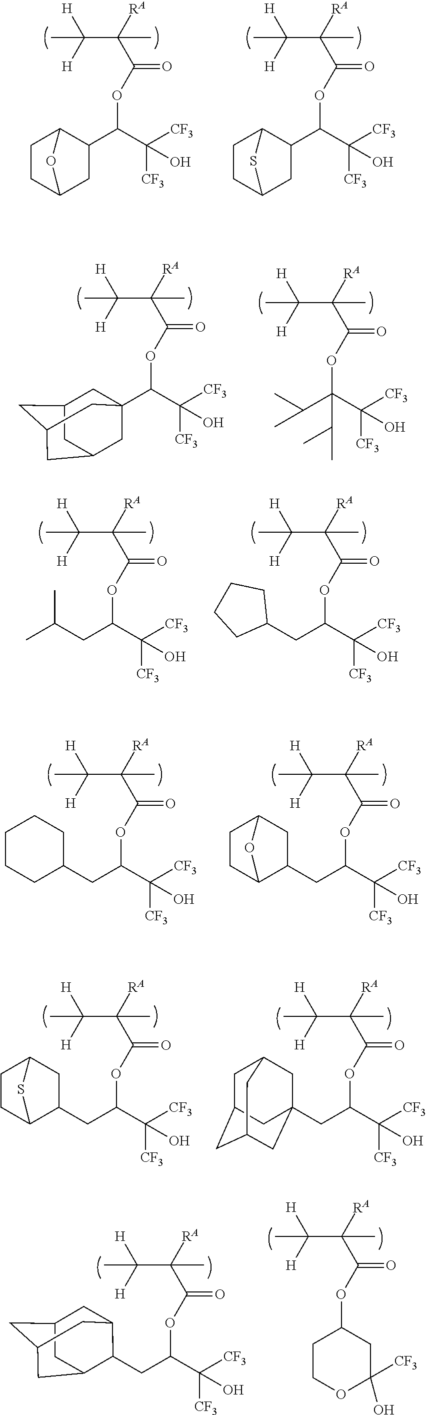

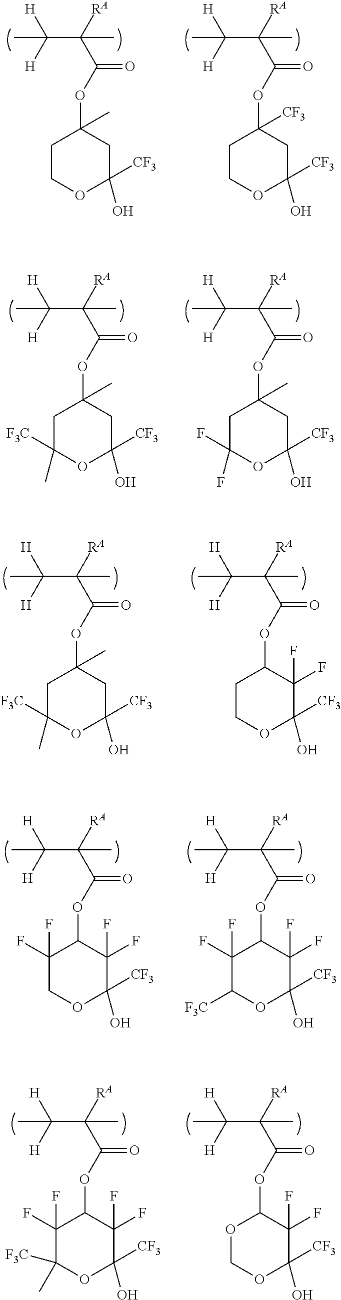

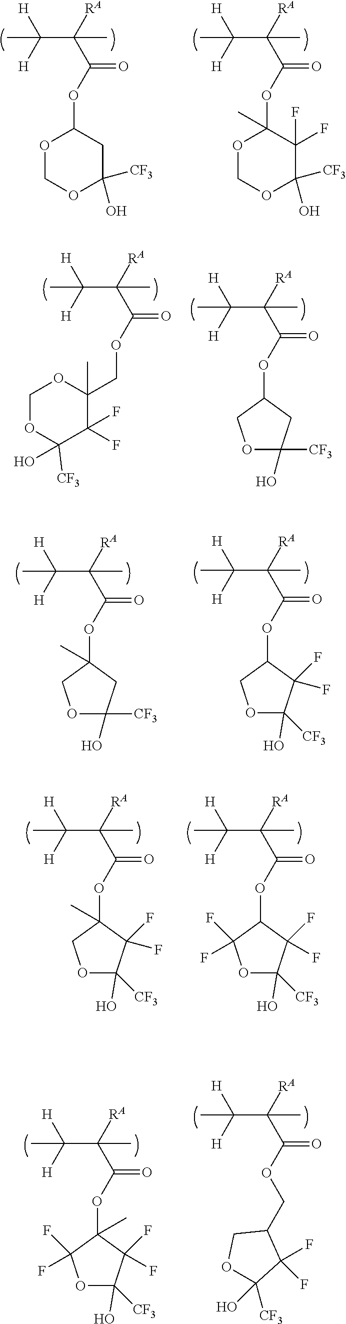

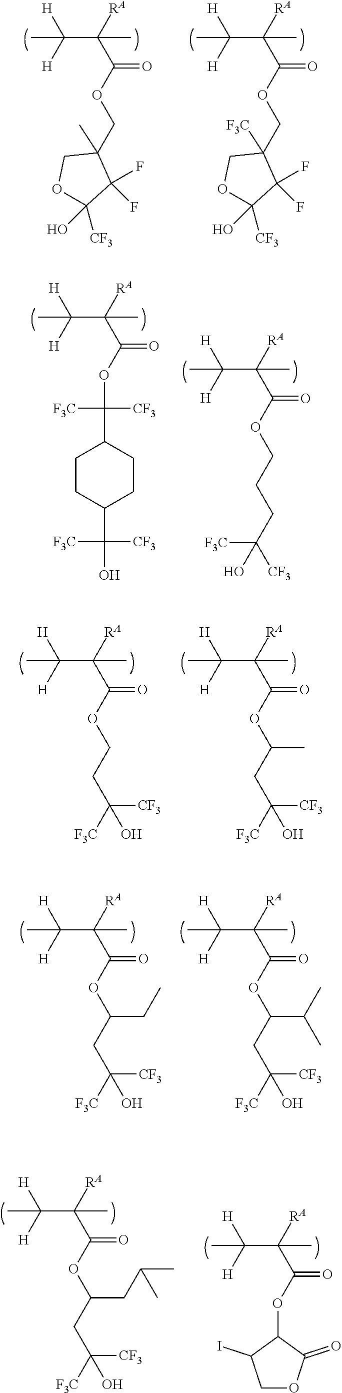

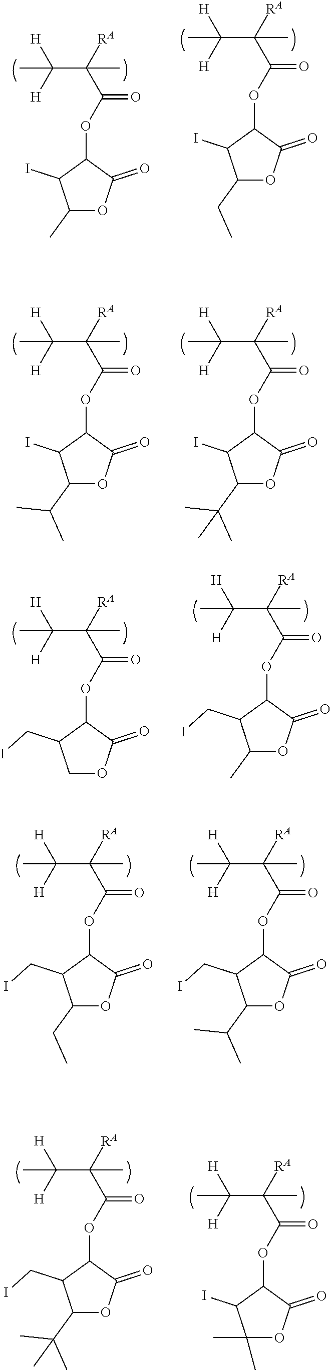

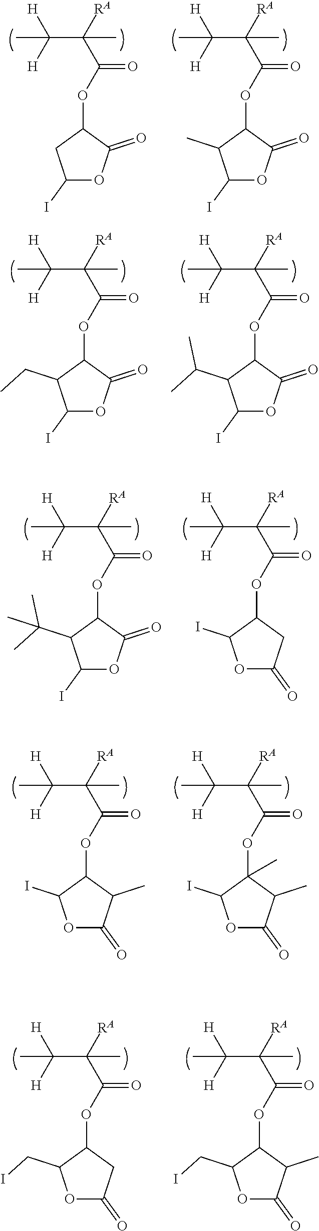

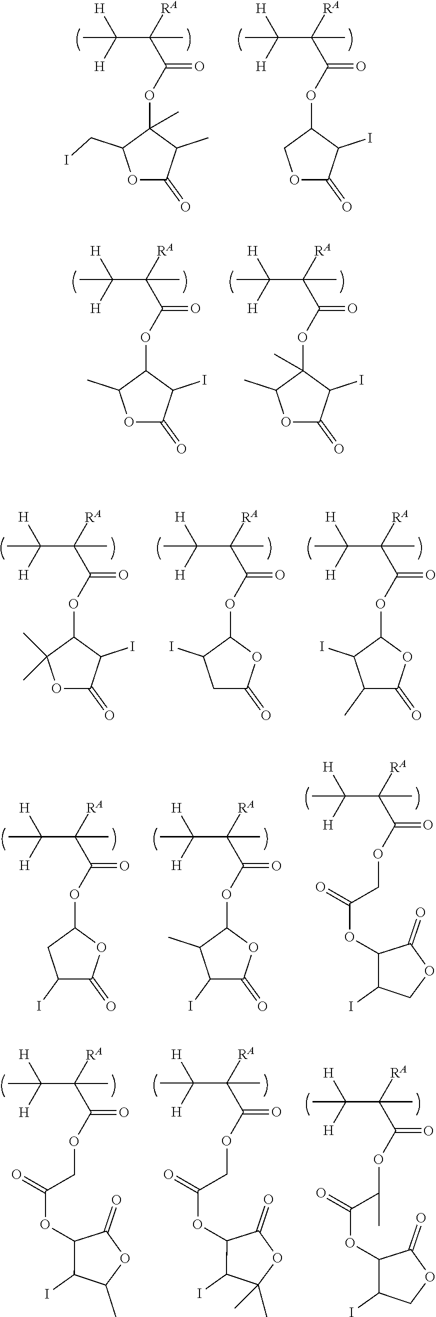

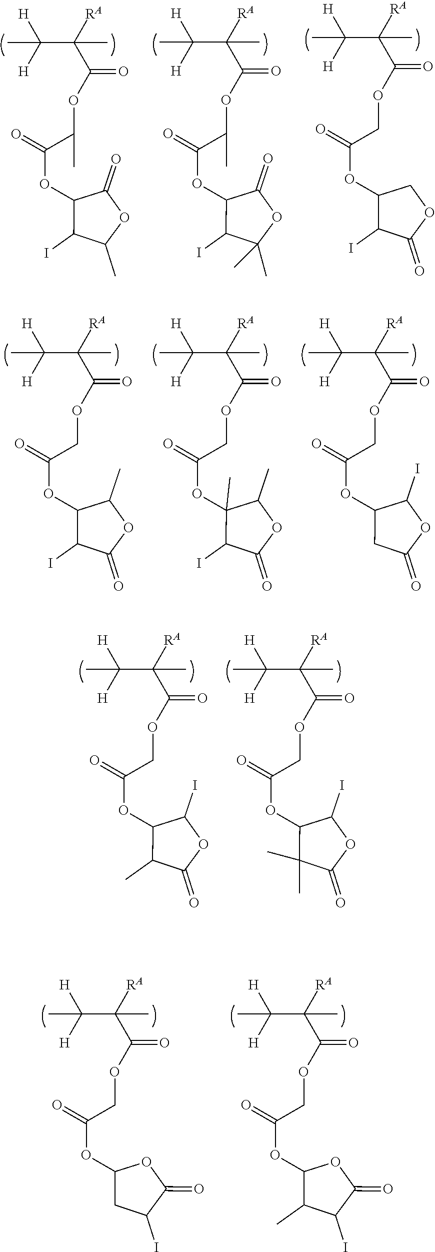

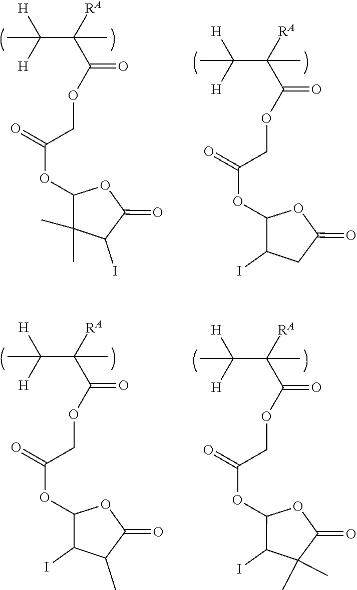

Examples of the repeat unit (b2) are shown below, but not limited thereto. Herein R^{A} is as defined above.

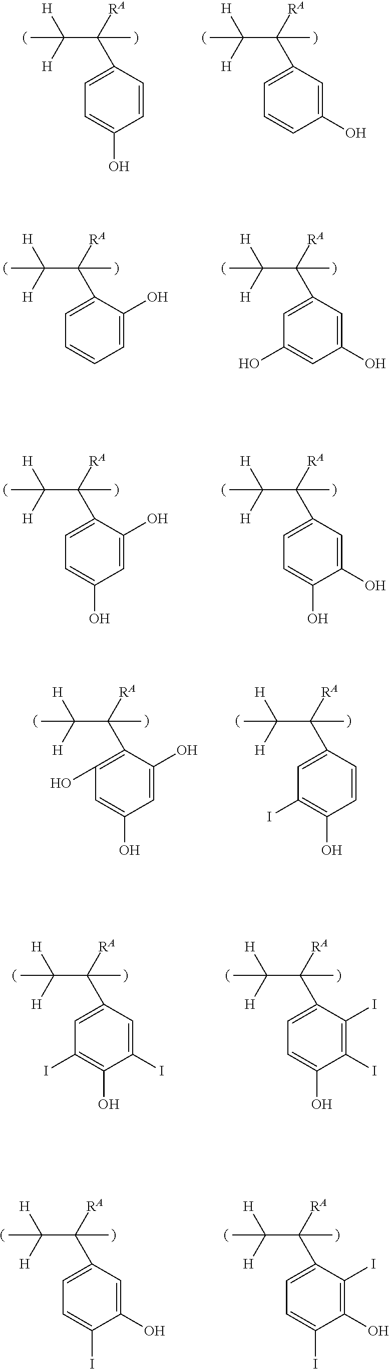

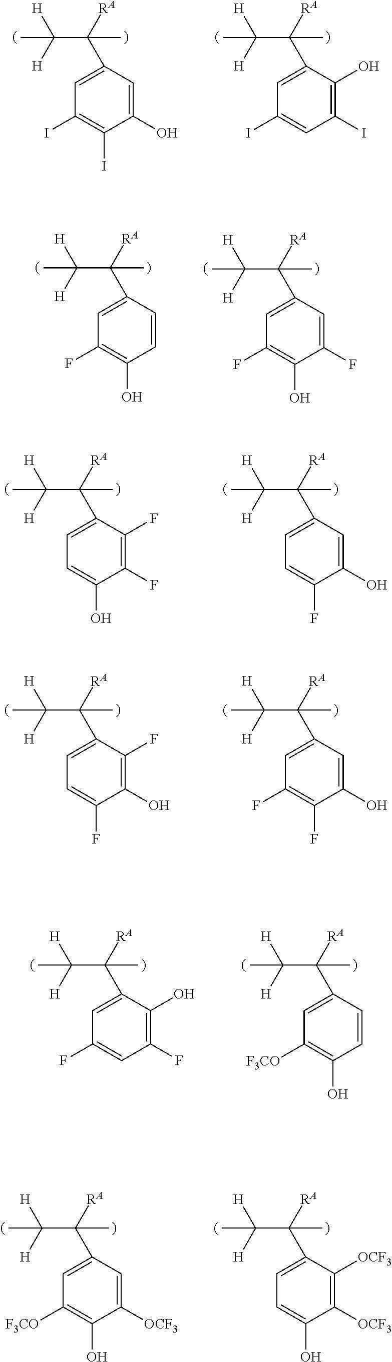

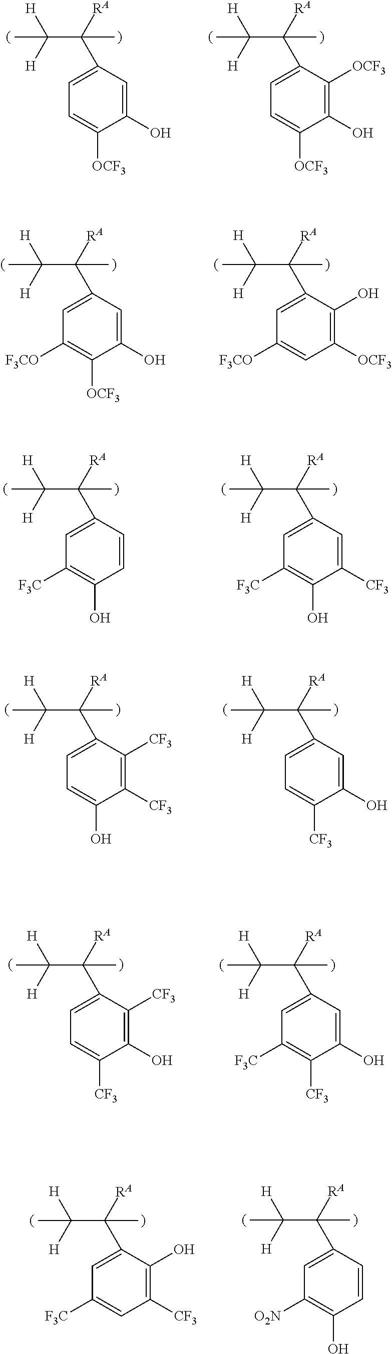

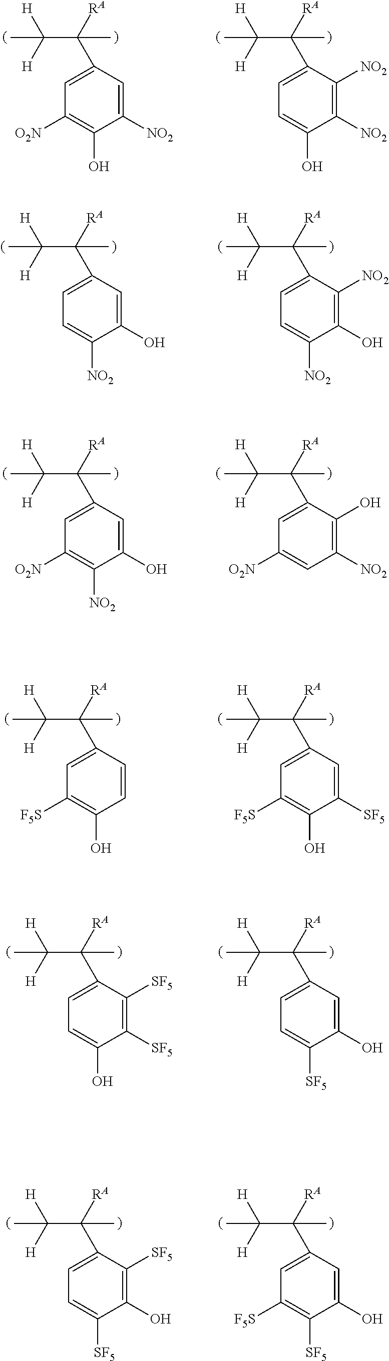

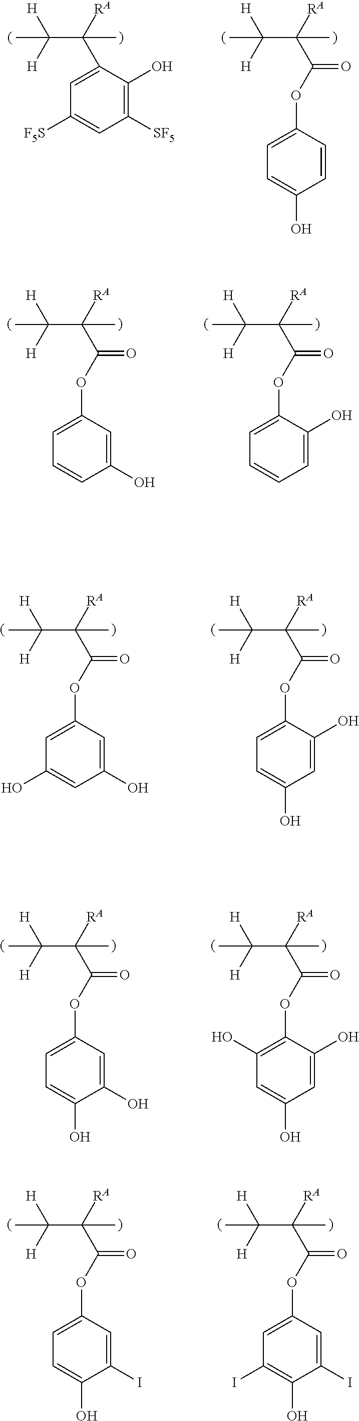

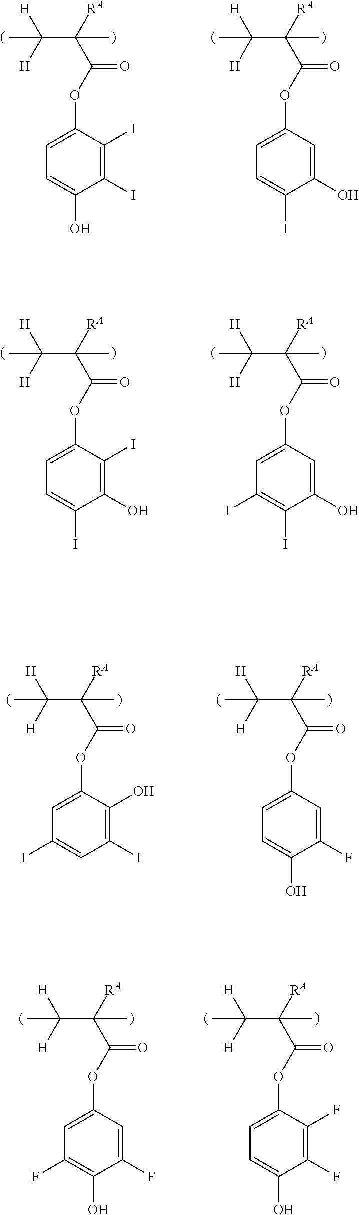

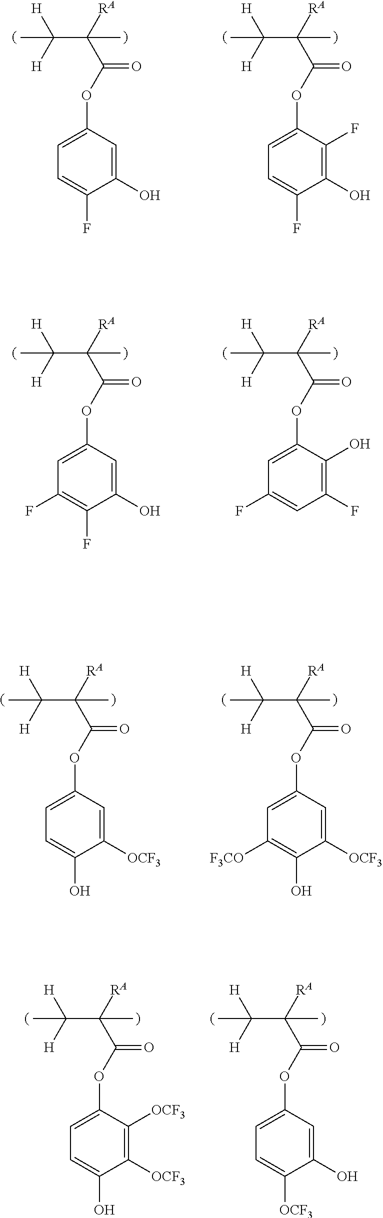

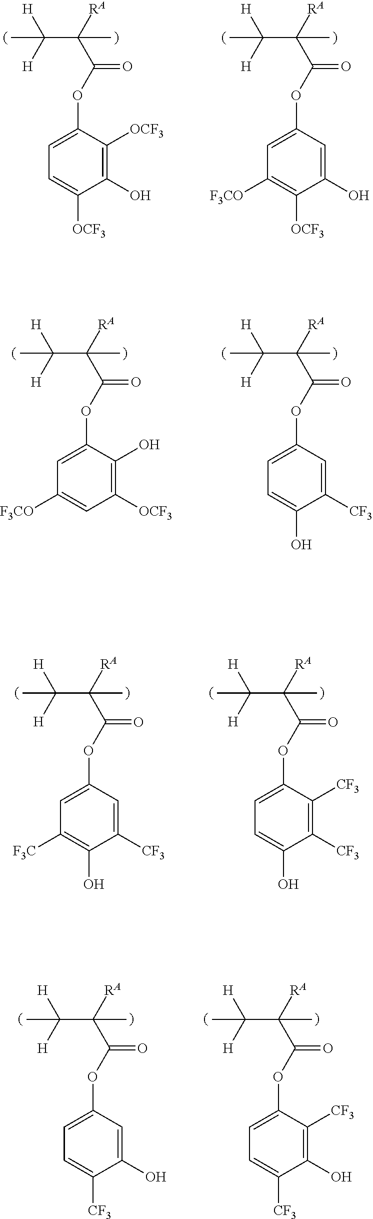

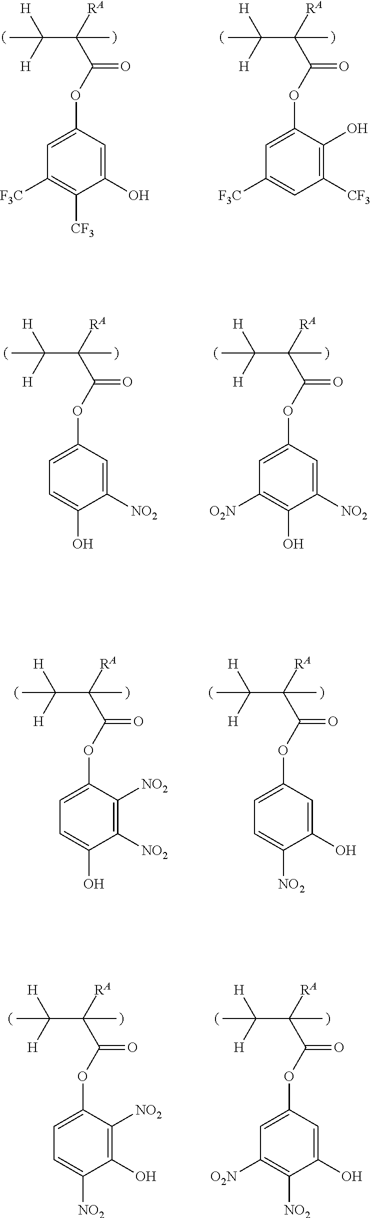

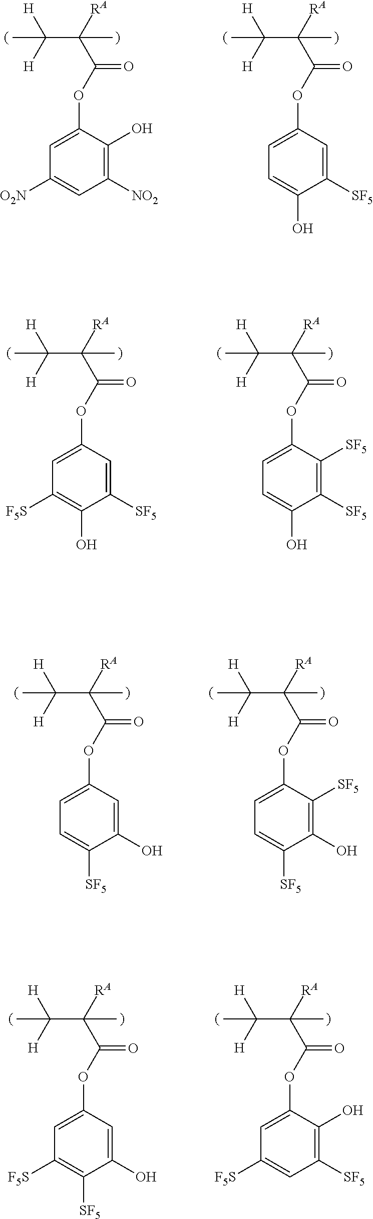

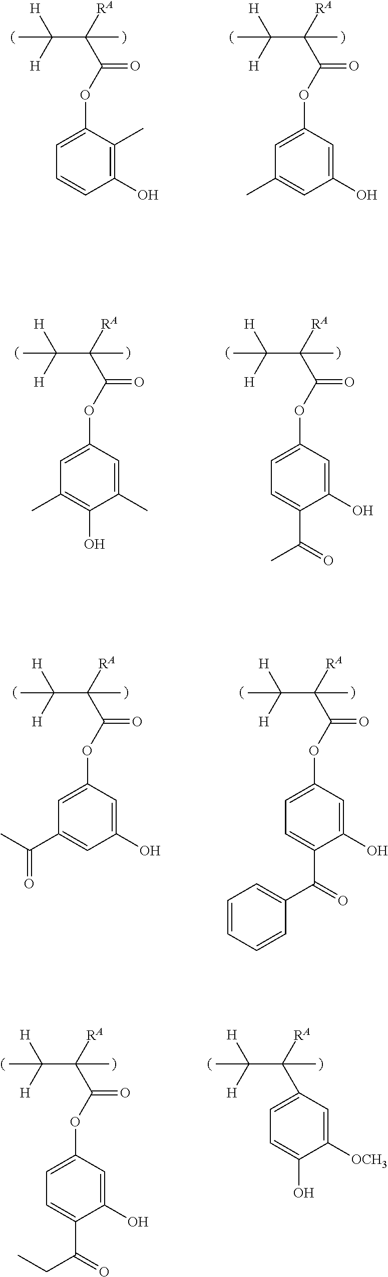

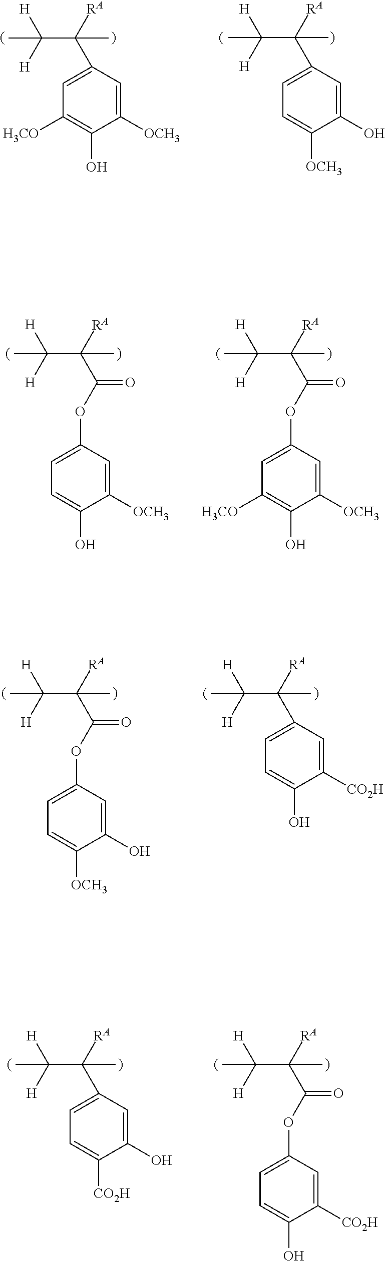

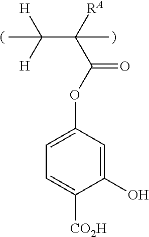

Of the repeat units (b1) and (b2), those units having a lactone ring as the polar group are preferred in the case of ArF lithography, and those units having a phenol site are preferred in the case of KrF, EB or EUV lithography.

The polymer may further comprise repeat units of at least one type selected from repeat units having the formula (c1), repeat units having the formula (c2), repeat units having the formula (c3), repeat units having the formula (c4), and repeat units having the formula (c5), which are referred to as repeat units (c1) to (c5), respectively.

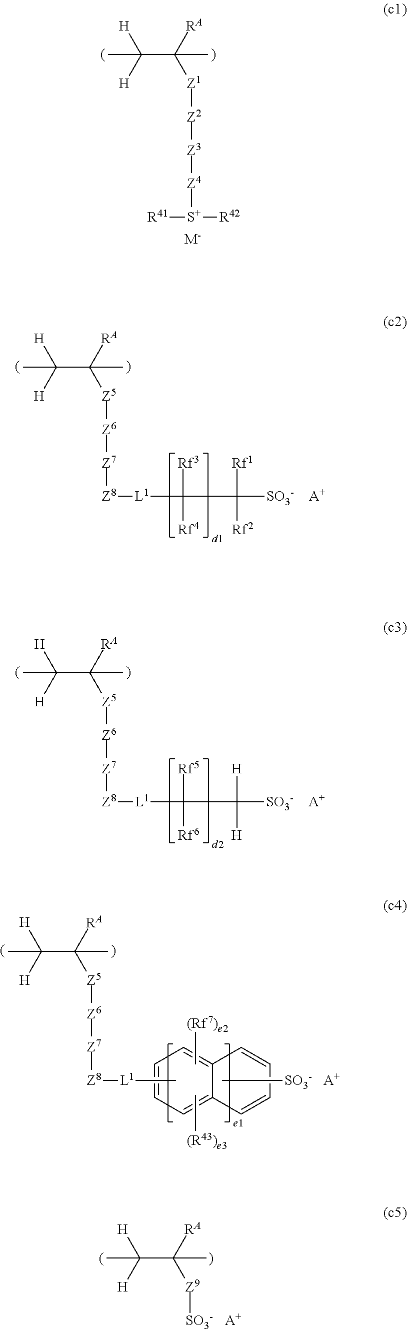

In formulae (c1) to (c5), R^{A} is each independently hydrogen, fluorine, methyl or trifluoromethyl.

Z¹ is a single bond or optionally substituted phenylene group. Z² is a single bond, **—C(═O)—O—Z²¹—, **—C(═O)—N(H)—Z²¹—, or **—O—Z²¹—. Z²¹ is a C₁-C₆ aliphatic hydrocarbylene group, phenylene group or a divalent group obtained by combining the foregoing, which may contain halogen, carbonyl moiety, ester bond, ether bond or hydroxy moiety. Z³ is a single bond, ether bond, ester bond, sulfonate ester bond, amide bond, sulfonamide bond, carbonate bond or carbamate bond. Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety.

Z⁵ is each independently a single bond, optionally substituted phenylene group, optionally substituted naphthylene group, *—C(═O)—O—Z⁵¹— or *—C(═O)—N(R)—Z⁵¹-751 is a C₁-C₁₀ aliphatic hydrocarbylene group, phenylene group or naphthylene group, the aliphatic hydrocarbylene group may contain halogen, hydroxy, ether bond, ester bond or lactone ring. R is hydrogen or a C₁-C₁₀ hydrocarbyl group which may contain a heteroatom.

Z⁶ is a single bond, ether bond, ester bond, sulfonate ester bond, amide bond, sulfonamide bond, carbonate bond or carbamate bond. Z⁷ is each independently a single bond, ***—Z⁷¹—C(═O)—O—, ***—C(═O)—N(R)—Z⁷¹— or ***—O—Z⁷¹-771 is a C₁-C₂₀ hydrocarbylene group which may contain a heteroatom. R is as defined above. Z⁸ is each independently a single bond, ****—Z⁸¹—C(═O)—O—, ****—C(—O)—N(R)—Z⁸¹— or ****—O—Z⁸¹—. Z⁸¹ is a C₁-C₂₀ hydrocarbylene group which may contain a heteroatom. R is as defined above.

Z⁹ is a single bond, methylene, ethylene, phenylene, fluorinated phenylene, trifluoromethyl-substituted phenylene, *—C(═O)—O—Z⁹¹—, *—C(═O)—N(R)—Z⁹¹— or *—O—Z⁹¹—. Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy. R is as defined above.

Herein, * designates a point of attachment to the carbon atom in the backbone, ** designates a point of attachment to Z¹, *** designates a point of attachment to Z⁶, **** designates a point of attachment to Z⁷.

The aliphatic hydrocarbylene group represented by Z²¹, Z⁵¹ and Z⁹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene group which may contain a heteroatom, represented by Z⁷¹ and Z⁸¹, may be saturated or unsaturated and straight, branched or cyclic. Examples thereof are shown below, but not limited thereto.

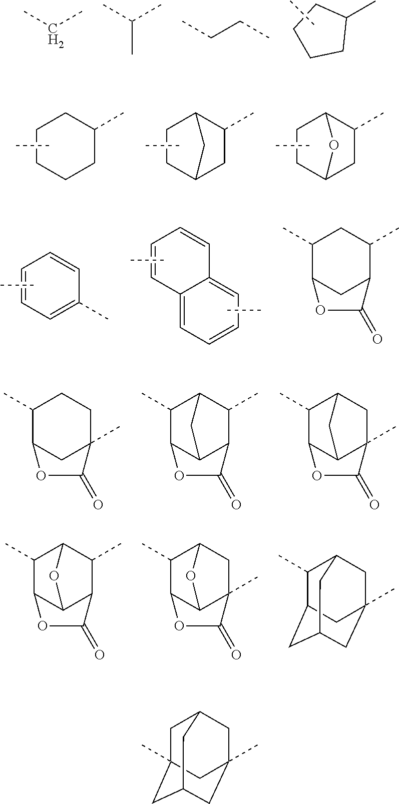

In formula (c1), R⁴¹ and R⁴² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl, and 2-phenylethyl, and combinations thereof. Of these, aryl groups are preferred. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

R⁴¹ and R⁴² may bond together to form a ring with the sulfur atom to which they are attached. Examples of the ring are shown below.

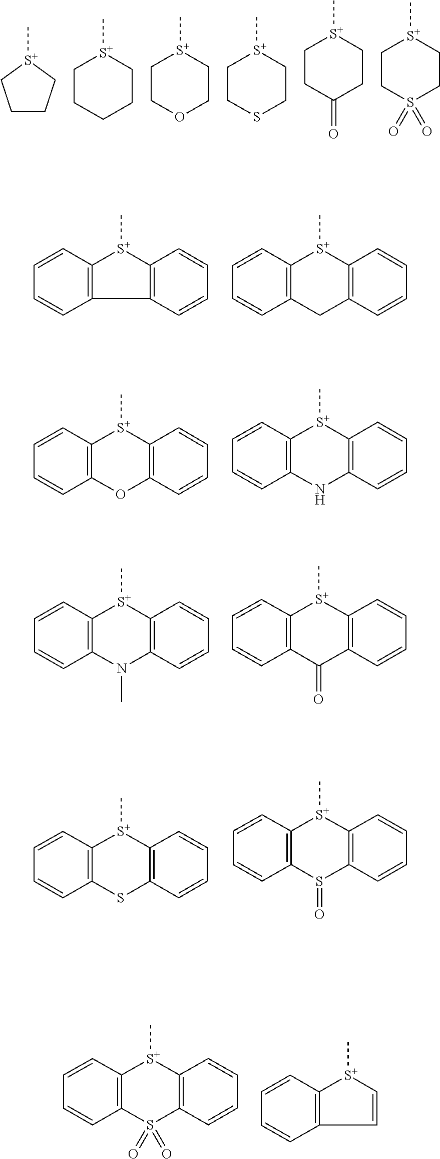

The broken line designates a point of attachment to Z⁴.

Examples of the cation in repeat unit (c1) are given below, but not limited thereto. Herein R^{A} is as defined above.

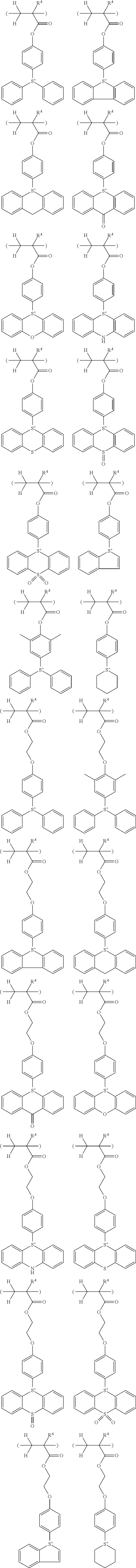

[chemical structure]

[chemical structure]

[chemical structure]

[chemical structure]

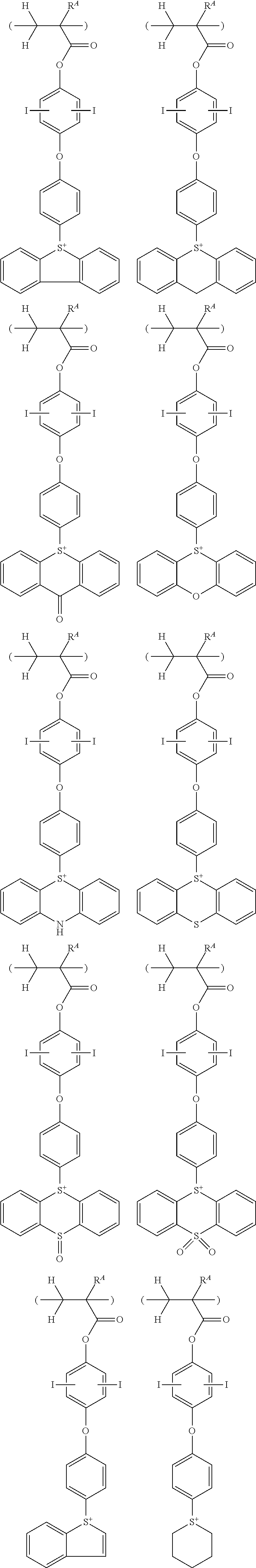

In formula (c1), M⁻ is a non-nucleophilic counter ion. Halide, sulfonate, imide and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl) methide and tris(perfluoroethylsulfonyl) methide.

Anions having the following formulae (c1-1) to (c1-4) are also useful as the non-nucleophilic counter ion.

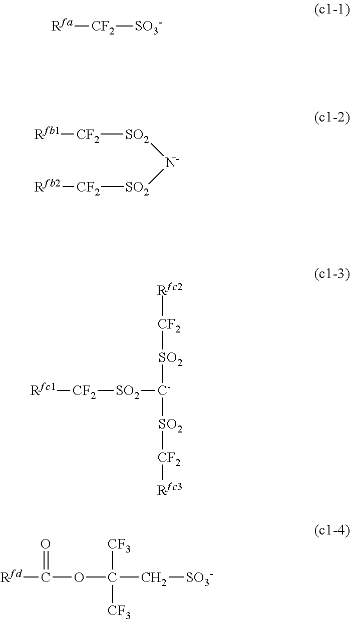

In formula (c1-1), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified below for the hydrocarbyl group R^{fa1} in formula (c1-1-1). In the hydrocarbyl group, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety.

Of the anions of formula (c1-1), an anion having the formula (c1-1-1) is preferred.

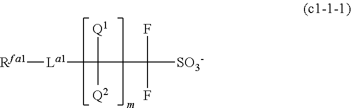

In formula (c1-1-1), Q¹ and Q² are each independently hydrogen, fluorine or a C₁-C₆ fluorinated saturated hydrocarbyl group. It is preferred for solvent solubility that at least one of Q¹ and Q² be trifluoromethyl. The subscript m is 0, 1, 2, 3 or 4, most preferably 1.

In formula (c1-1-1), R^{fa1} is a C₁-C₄₀ hydrocarbyl group which may contain a heteroatom. As the heteroatom, oxygen, nitrogen, sulfur and halogen atoms are preferred, with oxygen being most preferred. Of the hydrocarbyl groups, those groups of 6 to 30 carbon atoms are preferred from the aspect of achieving a high resolution in forming patterns of small feature size. The hydrocarbyl group may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₄₀ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₄₀ unsaturated aliphatic hydrocarbyl groups such as 2-propenyl and 3-cyclohexenyl; C₆-C₄₀ aryl groups such as phenyl, 1-naphthyl, 2-naphthyl and 9-fluorenyl; C₇-C₄₀ aralkyl groups such as benzyl and diphenylmethyl; C₇-C₄₀ aromatic ring-containing polycyclic hydrocarbyl groups such as 9,10-ethano-9,10-dihydroanthryl and 6,13-ethano-6,13-dihydropentacenyl; C₁₇-C₄₀ hydrocarbyl groups having steroid skeleton, and combinations thereof.

In the foregoing hydrocarbyl groups, some or all hydrogen may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (c1-1-1), Lal is a single bond, ether bond, ester bond, sulfonate ester bond, carbonate bond or carbamate bond. From the aspect of synthesis, an ether bond or ester bond is preferred, with the ester bond being more preferred.

Examples of the anion having formula (c1-1) are shown below, but not limited thereto. Herein Q¹ is as defined above.

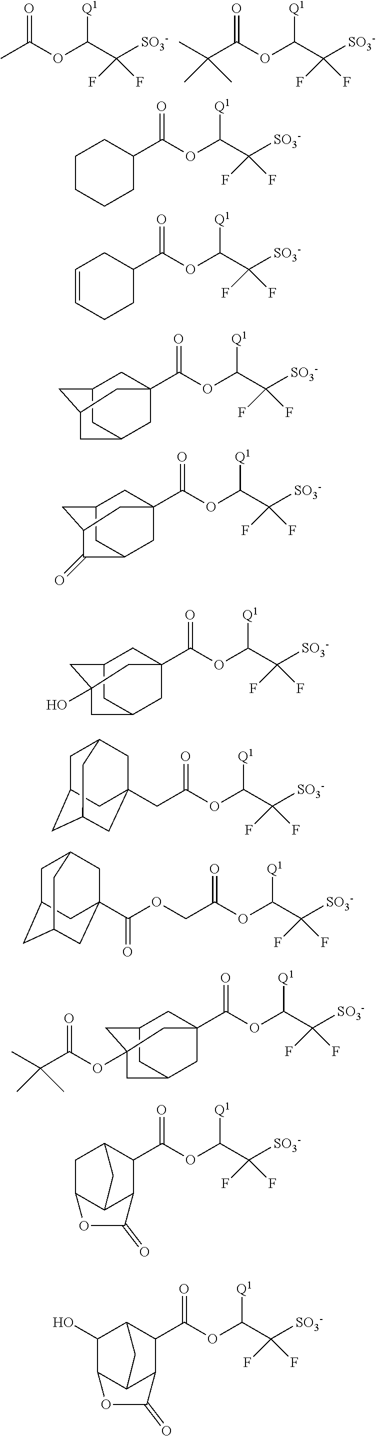

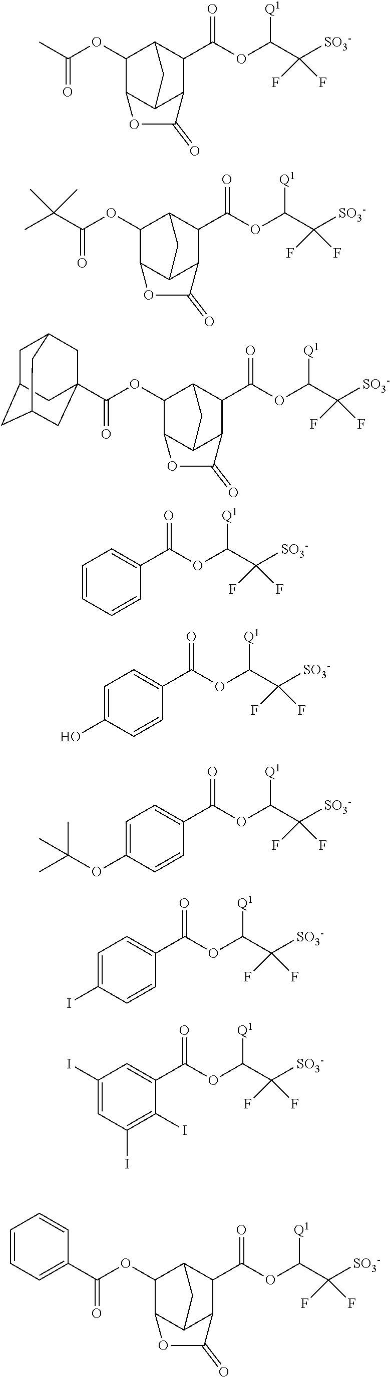

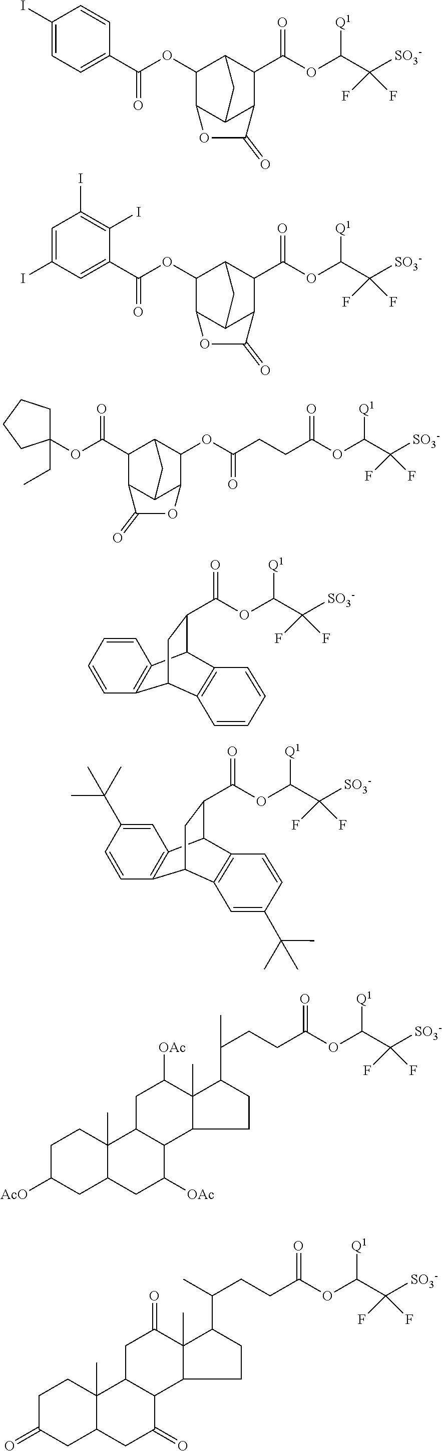

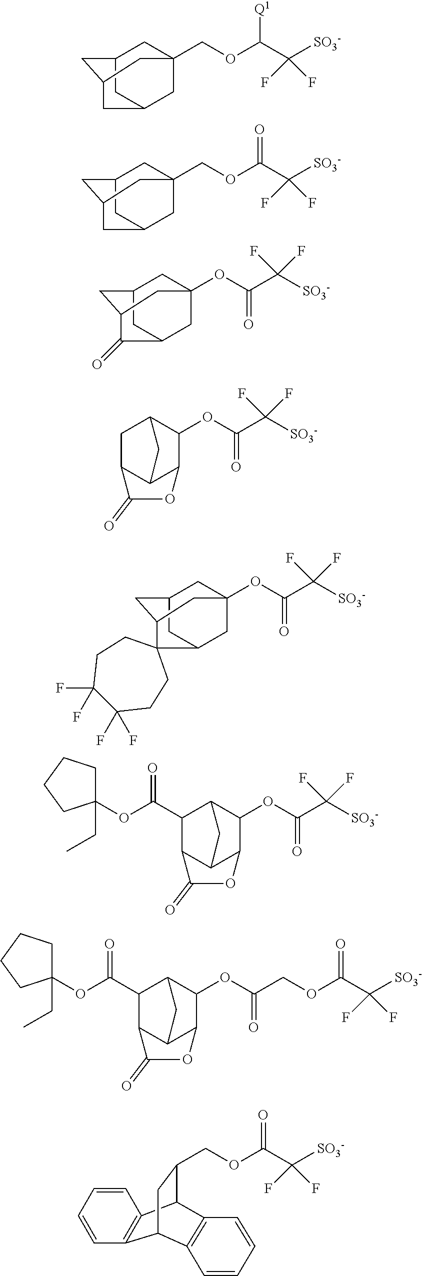

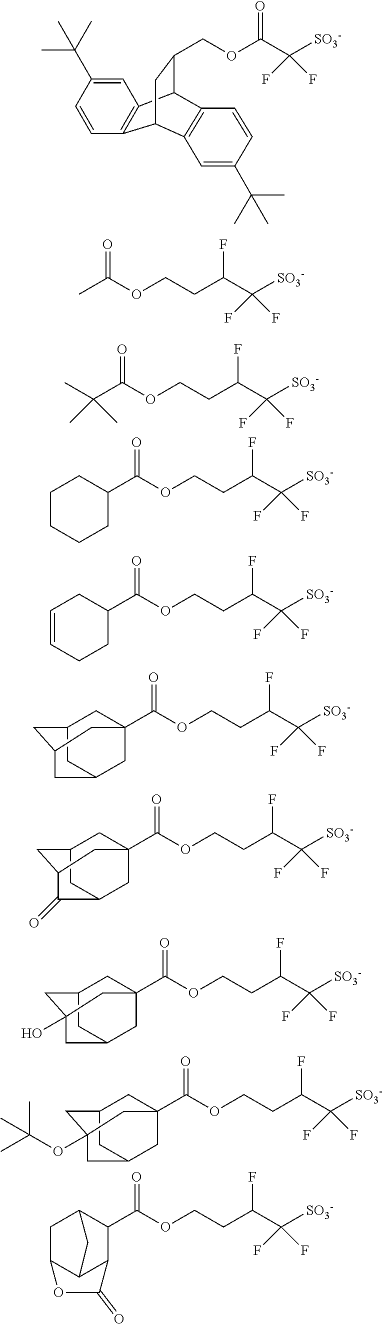

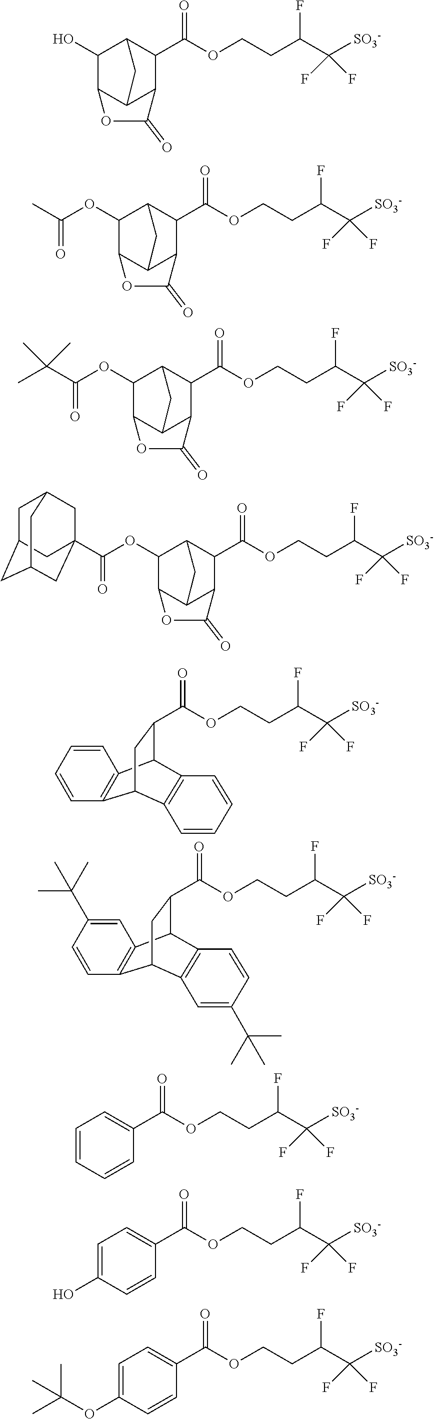

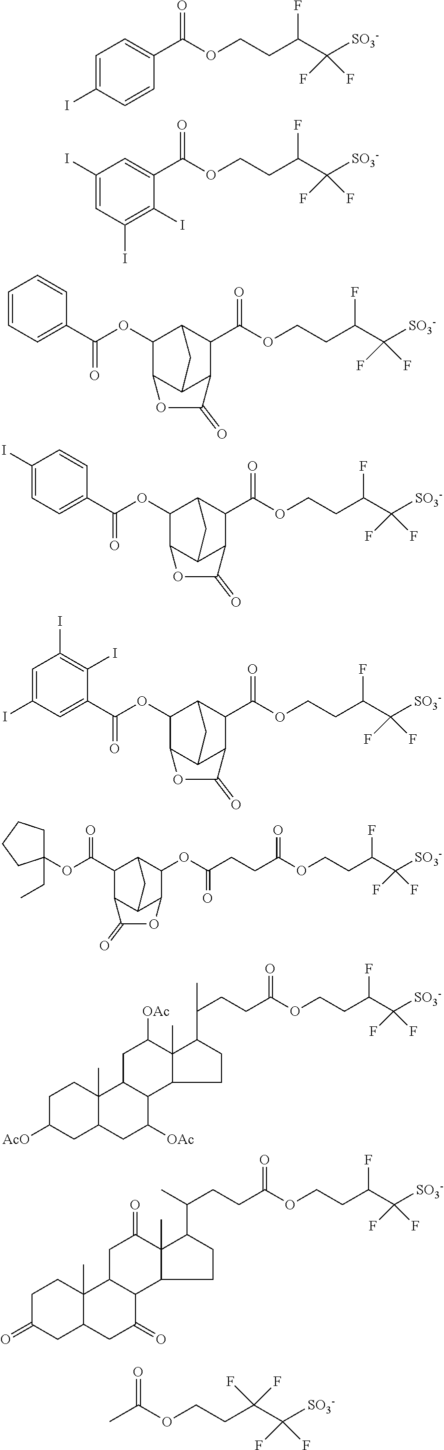

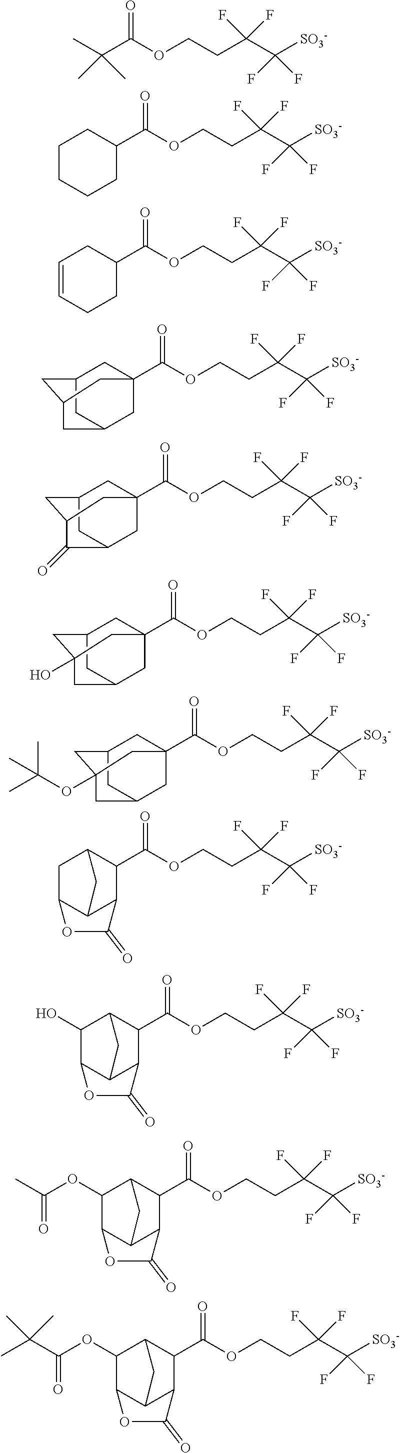

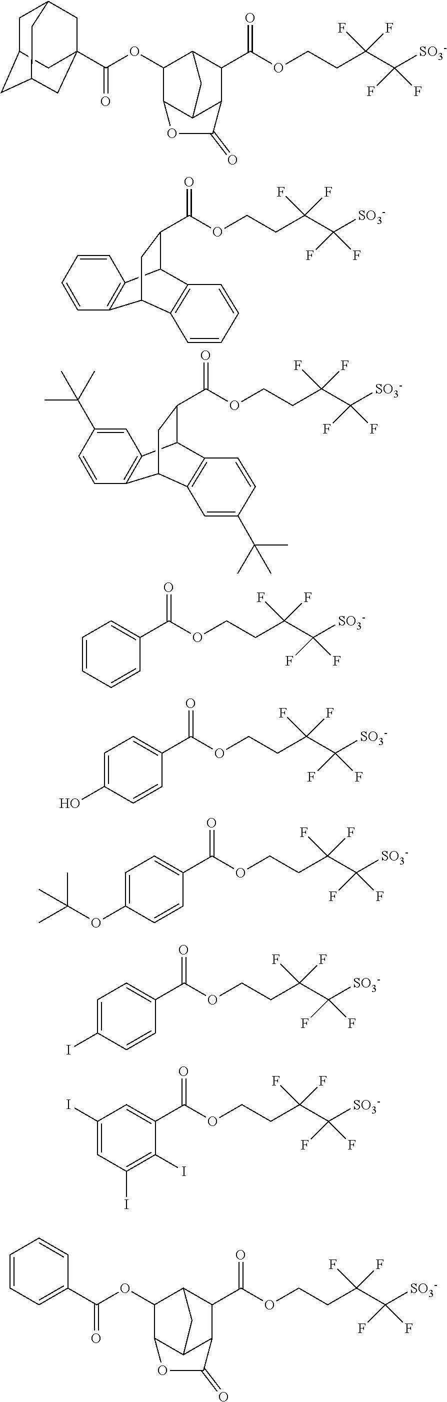

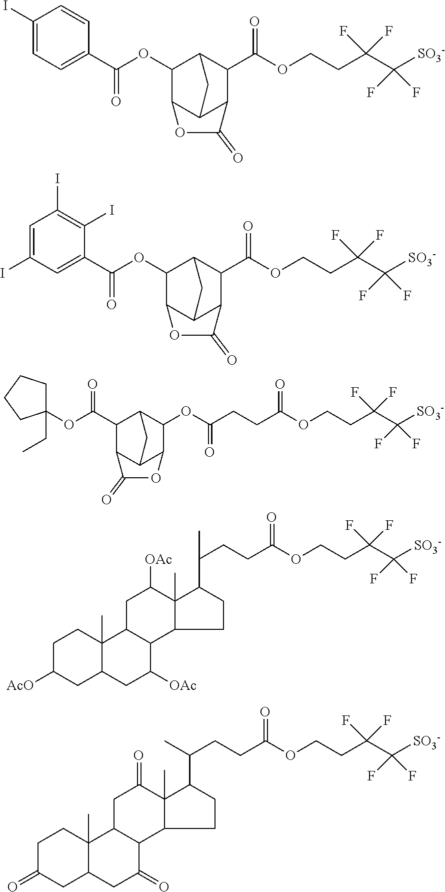

In formula (c1-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. It is preferred that a combination of R^{fb1} and R^{fb2} be a fluorinated ethylene or fluorinated propylene group.

In formula (c1-3), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In formula (c1-4), R^{fd} is a C₁-C₄₀ hydrocarbyl group which may contain a heteroatom. The hydrocarbyl group may be saturated or unsaturated and straight, branched or cyclic. Examples thereof are as exemplified above for R^{fa1}. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety.

Examples of the anion having formula (c1-4) are shown below, but not limited thereto.

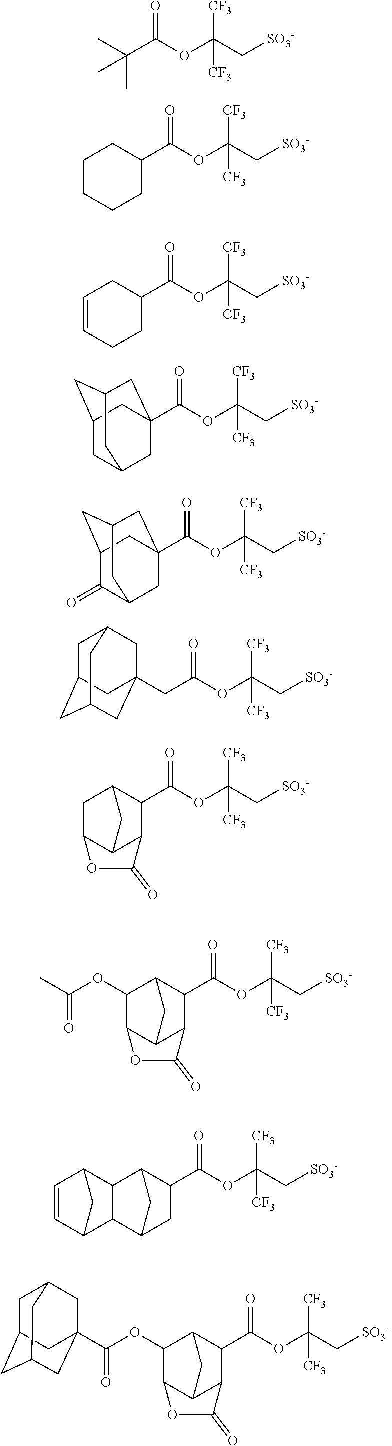

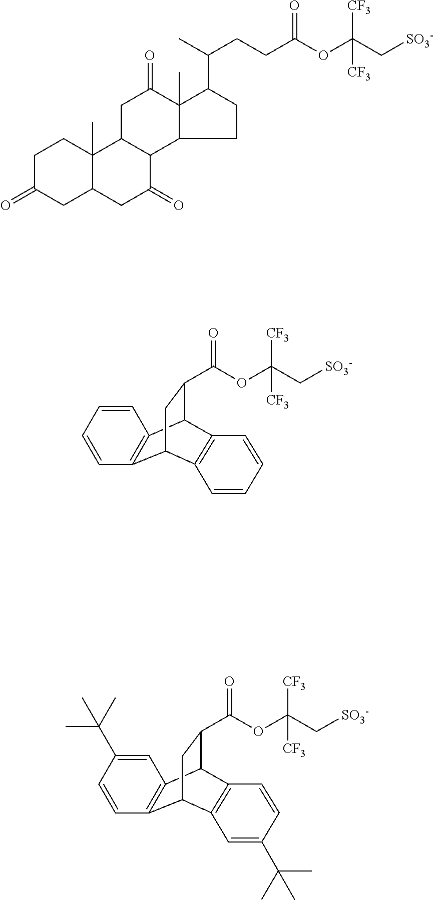

Anions having an iodized or brominated aromatic ring are also useful as the non-nucleophilic counter ion. These anions have the formula (c1-5).

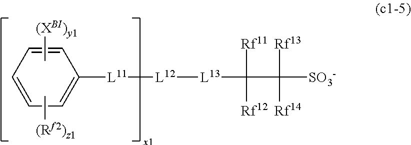

In formula (c1-5), x1 is 1, 2 or 3, y1 is 1, 2, 3, 4 or 5, z1 is 0, 1, 2 or 3, and y1+z1 is from 1 to 5. Preferably, y1 is 1, 2 or 3, more preferably 2 or 3, and z1 is 0, 1 or 2.

X^{BI} is iodine or bromine. A plurality of X^{BI} may be identical or different when x1 and/or y1 is 2 or more.

L¹¹ is a single bond, ether bond, ester bond, sulfonate ester bond, carbonate bond, carbamate bond or a C₁-C₆ saturated hydrocarbylene group in which some —CH₂— may be replaced by an ether bond or ester bond. The saturated hydrocarbylene group may be straight, branched or cyclic.

L¹² is a single bond or a C₁-C₂₀ hydrocarbylene group which may contain a heteroatom, when x1=1. L¹² is a C₁-C₂₀ (x1+1)-valent hydrocarbon group which may contain a heteroatom, when x1=2 or 3.

The C₁-C₂₀ hydrocarbylene group L¹² may be saturated or unsaturated and straight, branched or cyclic. Examples thereof include C₁-C₂₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, and dodecane-1,12-diyl; C₃-C₂₀ cyclic saturated hydrocarbylene groups such as cyclopentanediyl, cyclohexanediyl, norbornanediyl, adamantanediyl and tricyclo[5.2.1.0^{2,6}]decanediyl; C₂-C₂₀ unsaturated aliphatic hydrocarbylene groups such as vinylene and propene-1,3-diyl; C₆-C₂₀ arylene groups such as phenylene, naphthylene, and anthracenediyl; C₇-C₂₀ aromatic ring-containing polycyclic hydrocarbylene groups such as 9,10-ethano-9,10-dihydroanthracenediyl and 6,13-ethano-6,13-dihydropentacene; and combinations thereof. The C₁-C₂₀ (x1+1)-valent hydrocarbon group L¹² may be saturated or unsaturated and straight, branched or cyclic. Examples thereof include the above-mentioned examples of the C₁-C₂₀ hydrocarbylene group from which one or two hydrogen atoms are eliminated.

In the hydrocarbylene and (x1+1)-valent hydrocarbon groups, some or all of the hydrogen atoms may be substituted by a moiety containing a heteroatom such as oxygen, sulfur, nitrogen or halogen, or some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

In formula (c1-5), L¹³ is a single bond, ether bond, ester bond, sulfonate ester bond, carbonate bond or carbamate bond.

In formula (c1-5), R^{fe} is hydroxy, carboxy, fluorine, chlorine, bromine, amino group, or a C₁-C₂₀ hydrocarbyl, C₁-C₂₀ hydrocarbyloxy, C₁-C₂₀ hydrocarbylthio, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, iodine, hydroxy, amino, ester bond or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x1 and/or z1 is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having formula (c1-5) are shown below, but not limited thereto. X^{BI} is as defined above.

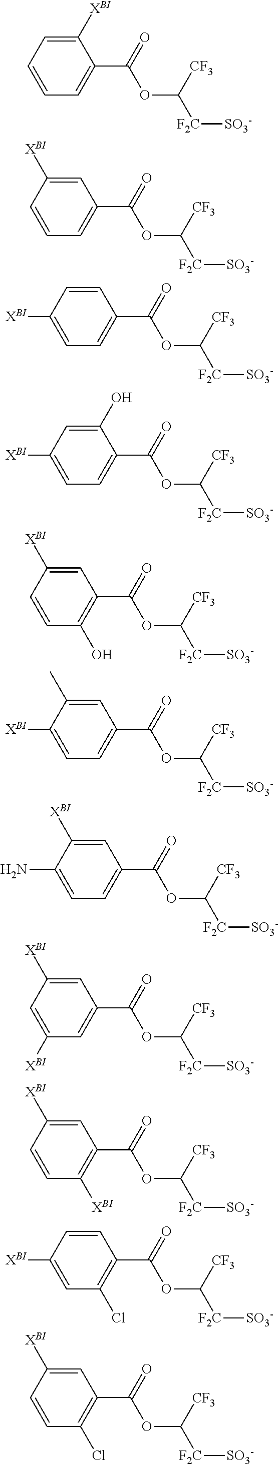

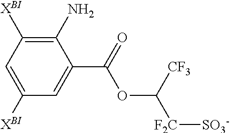

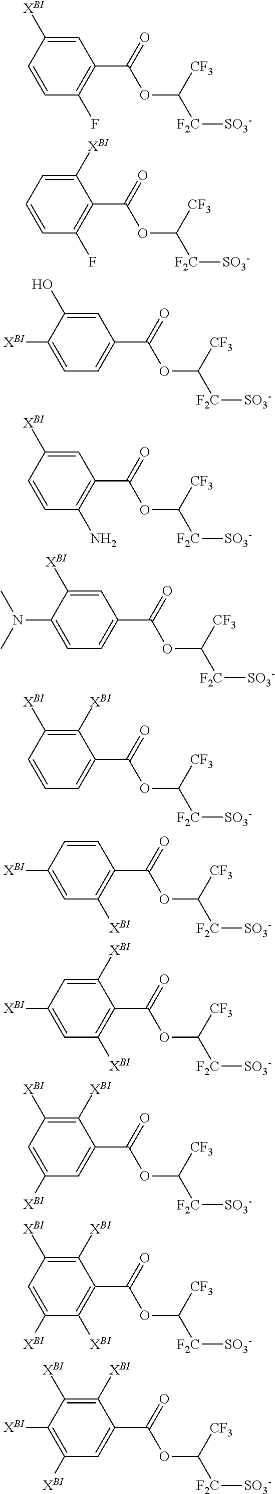

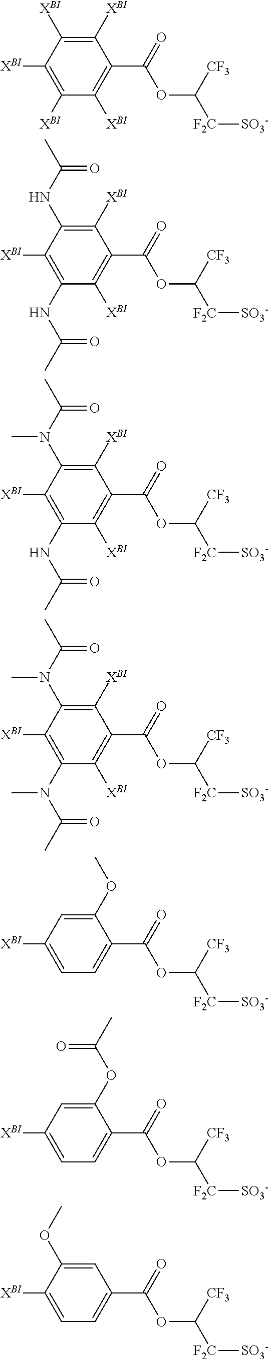

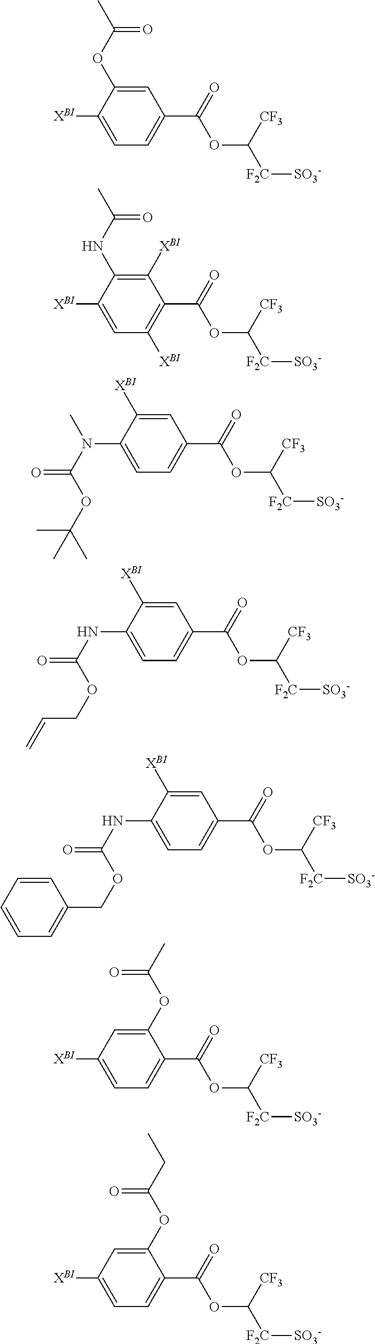

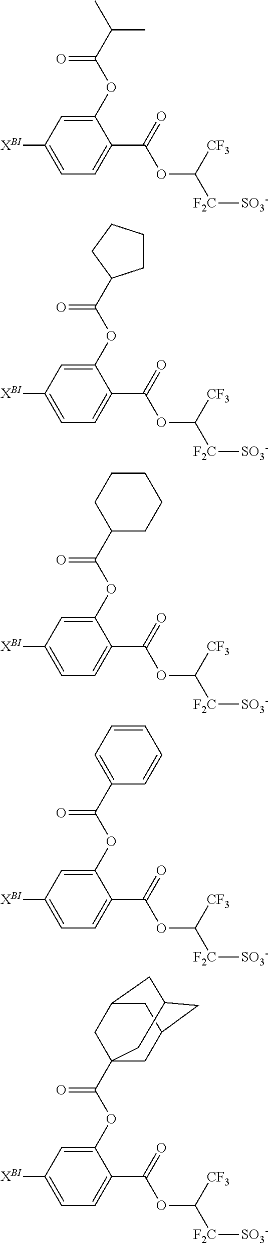

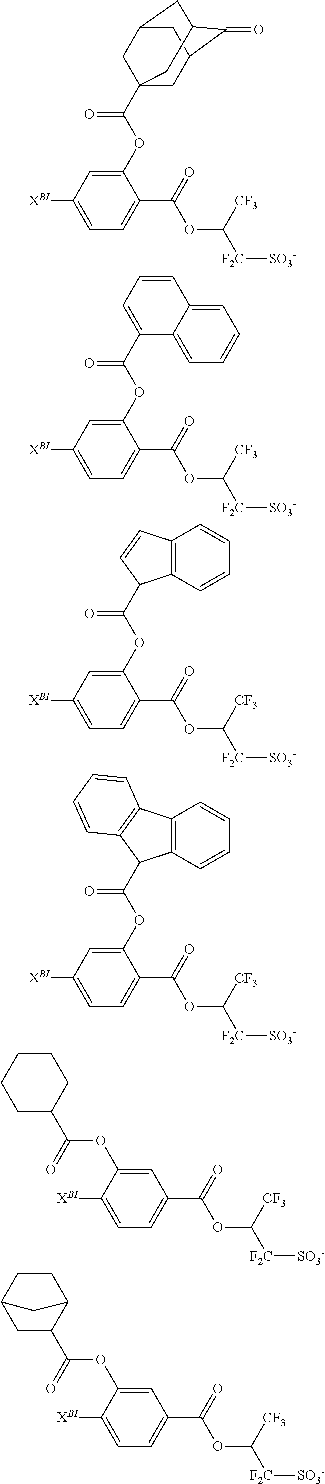

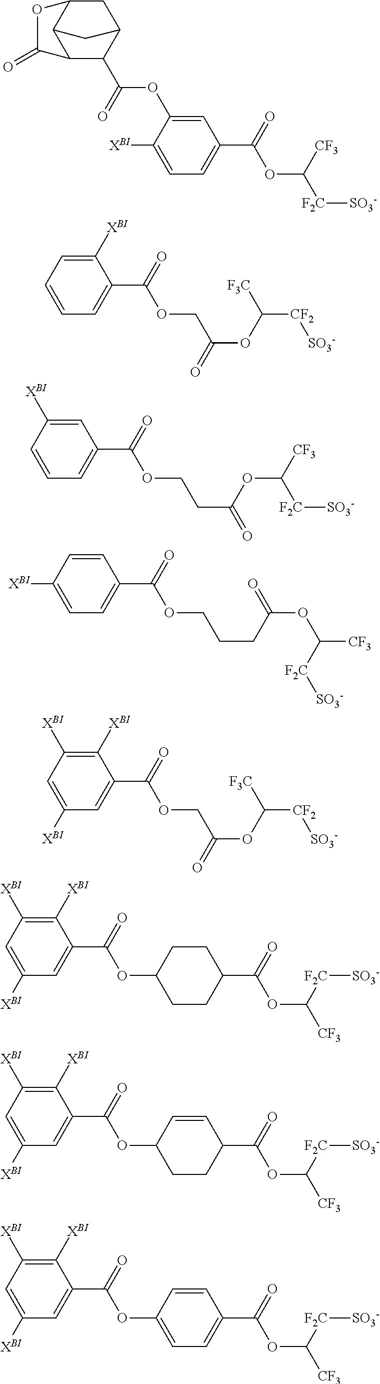

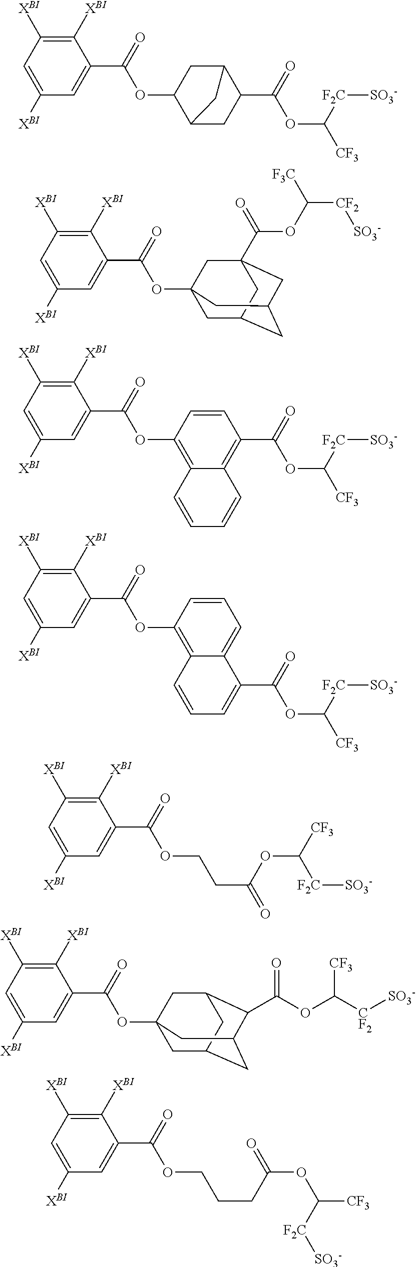

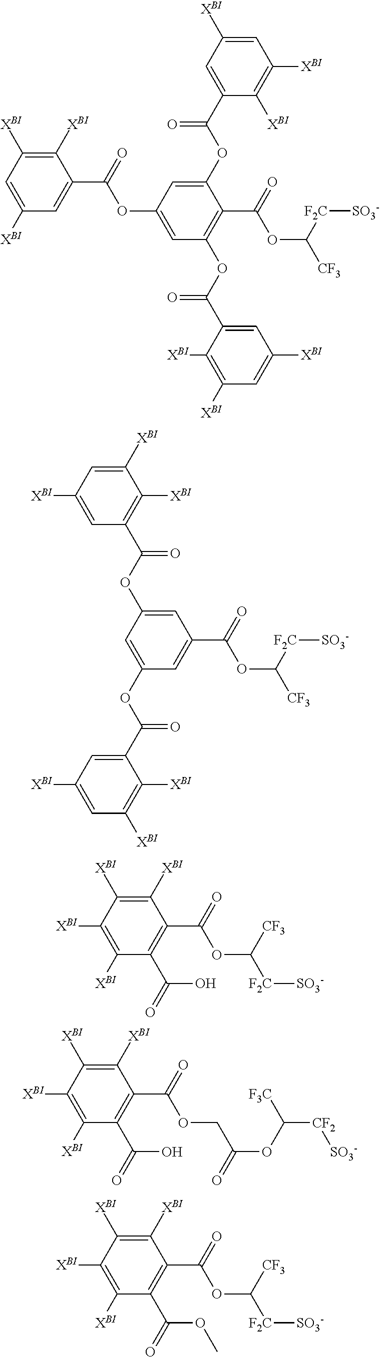

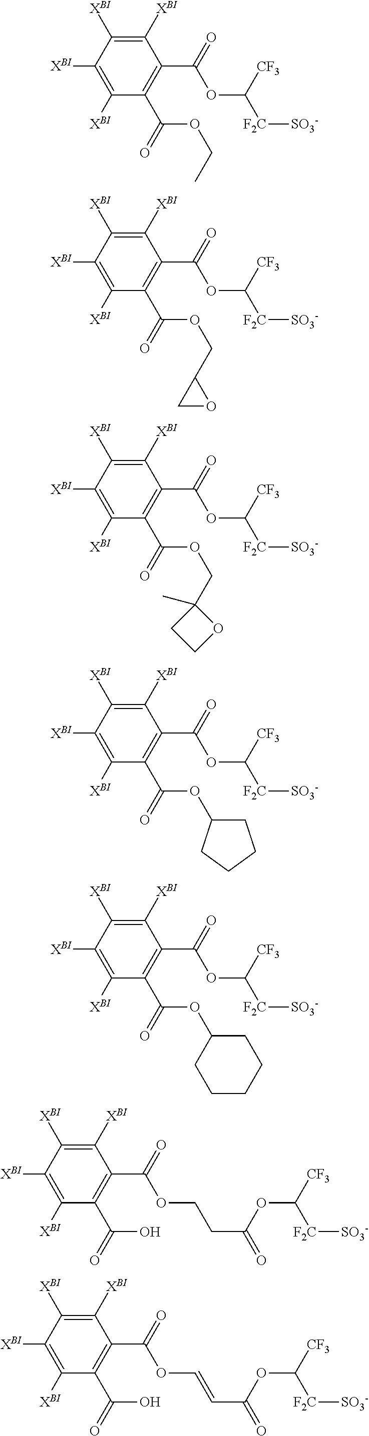

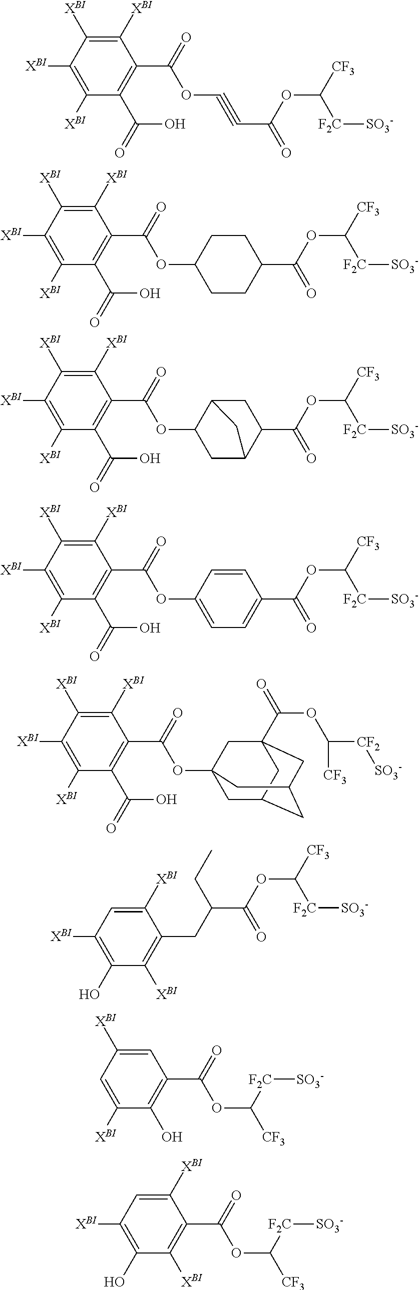

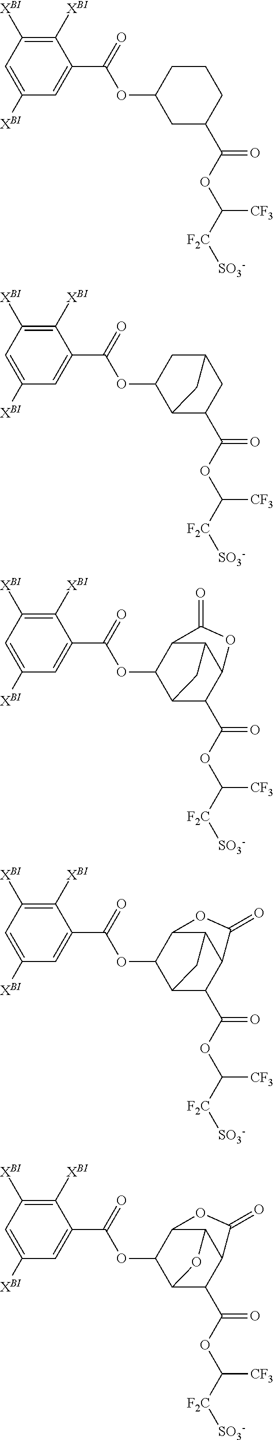

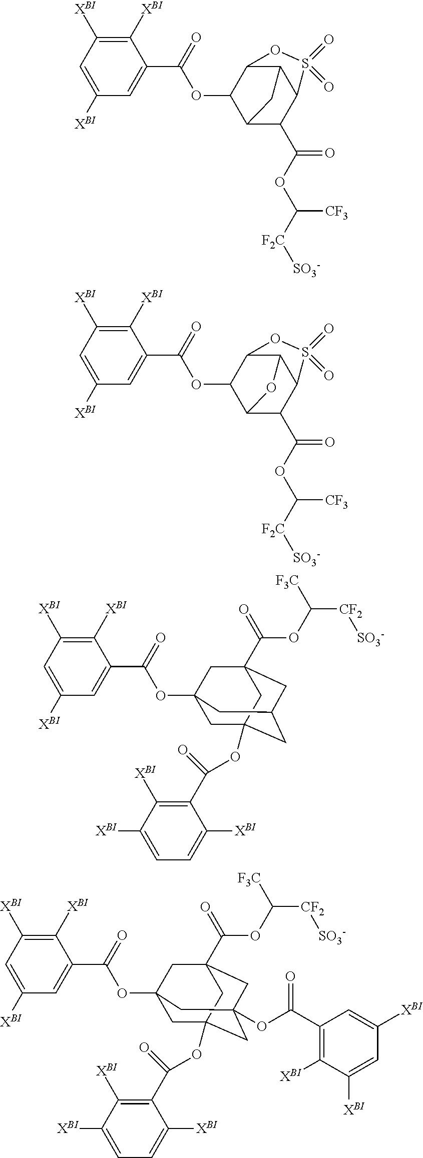

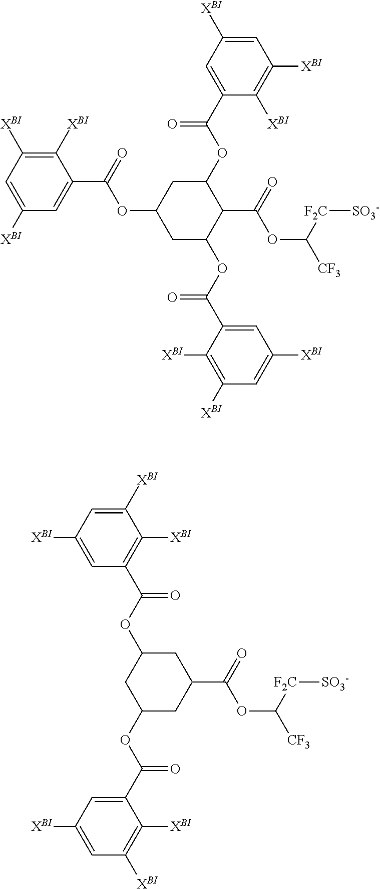

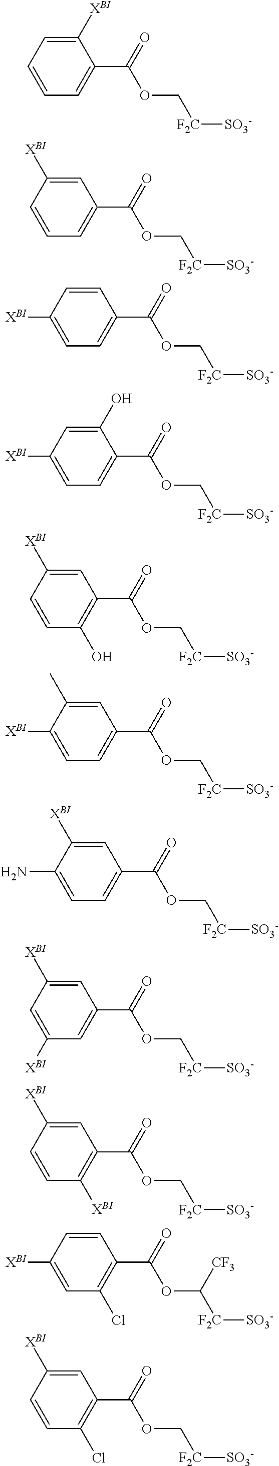

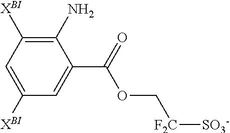

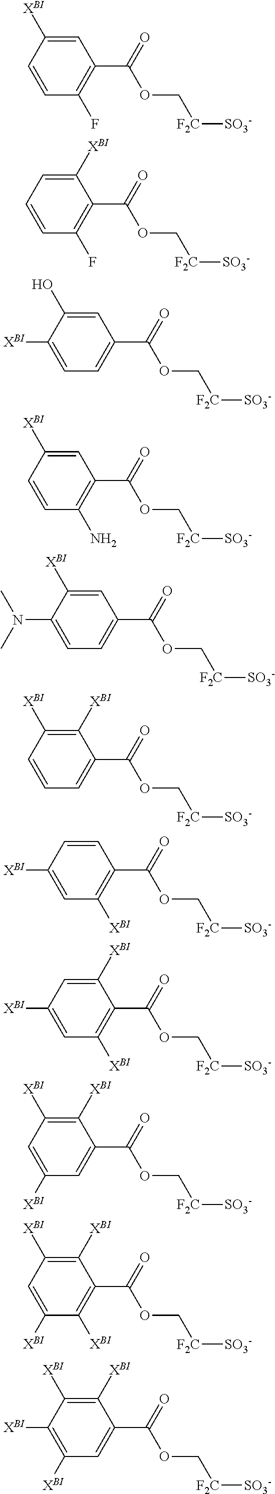

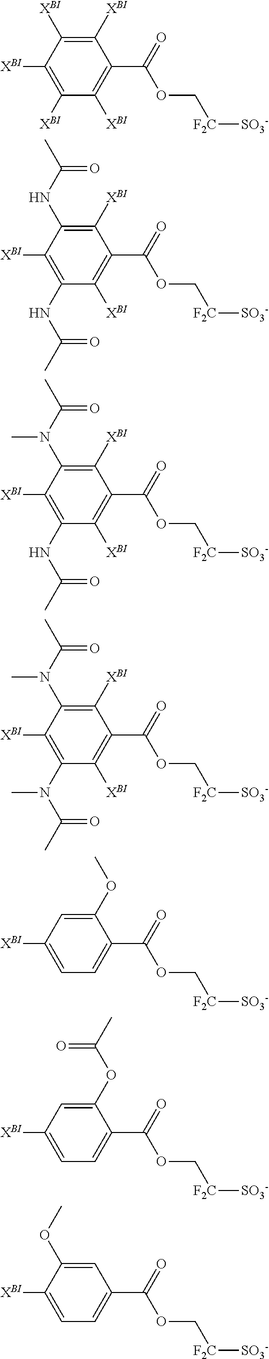

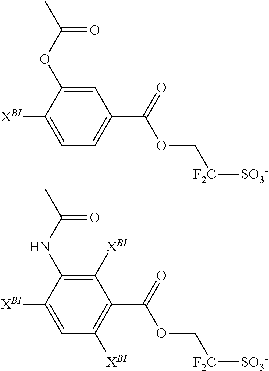

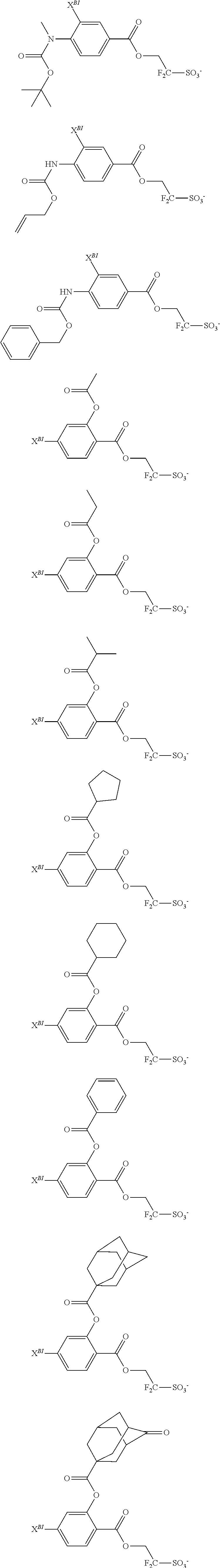

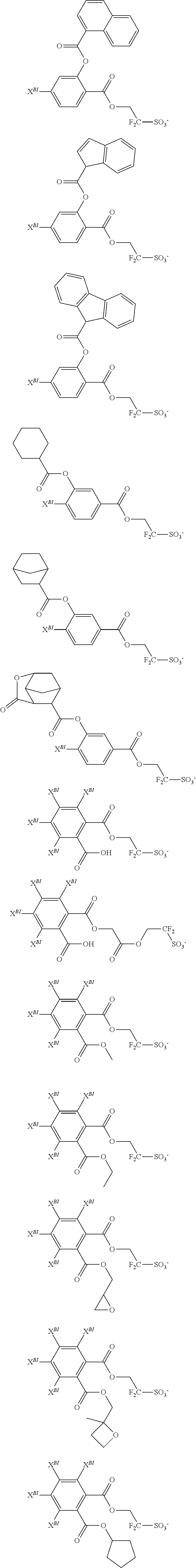

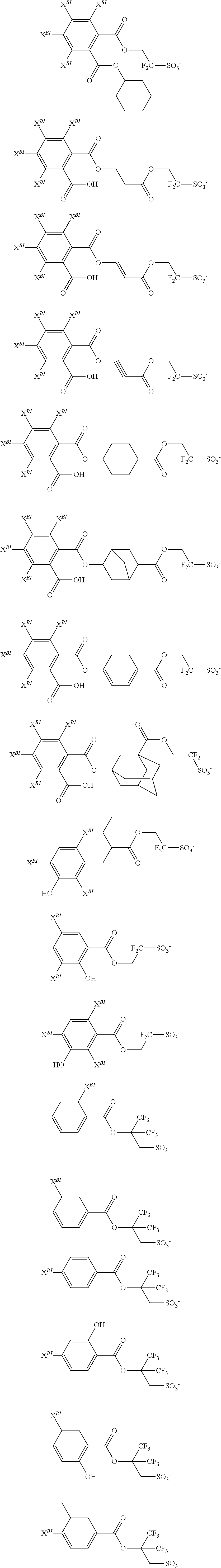

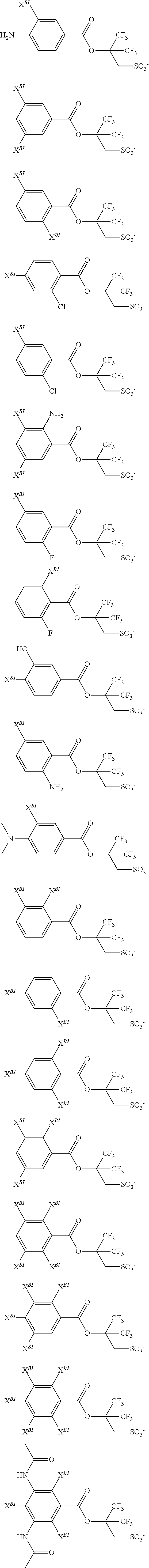

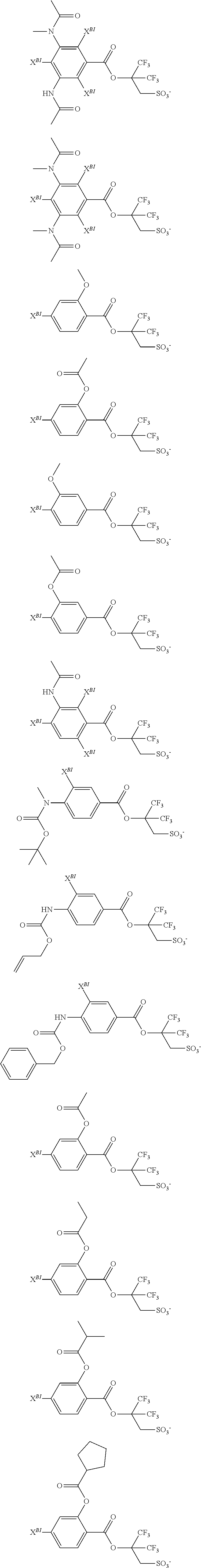

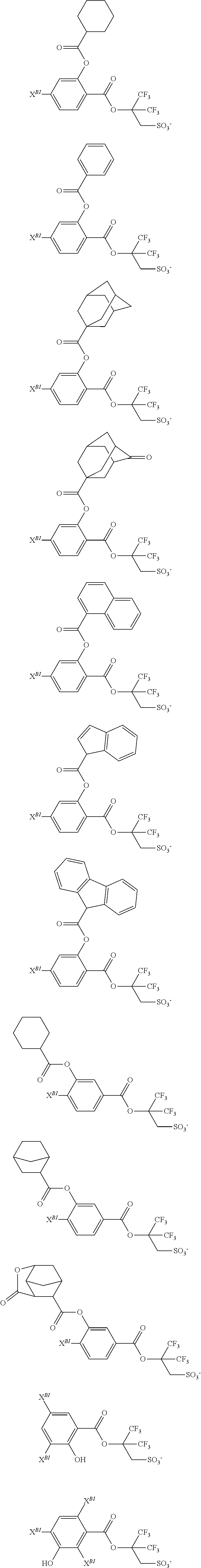

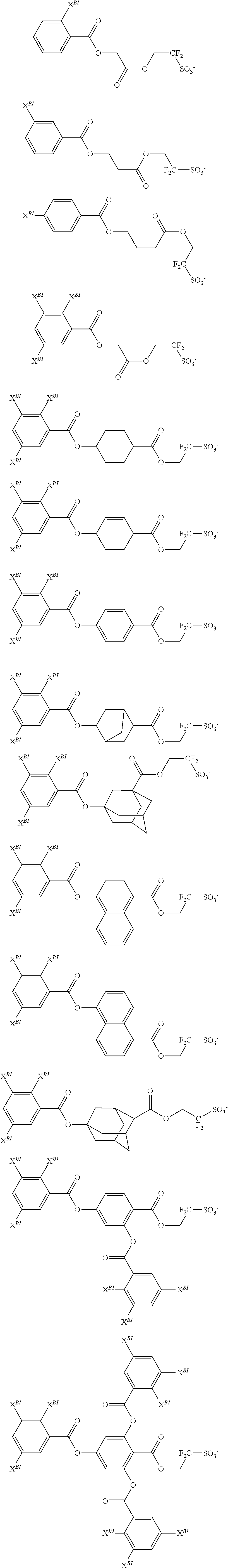

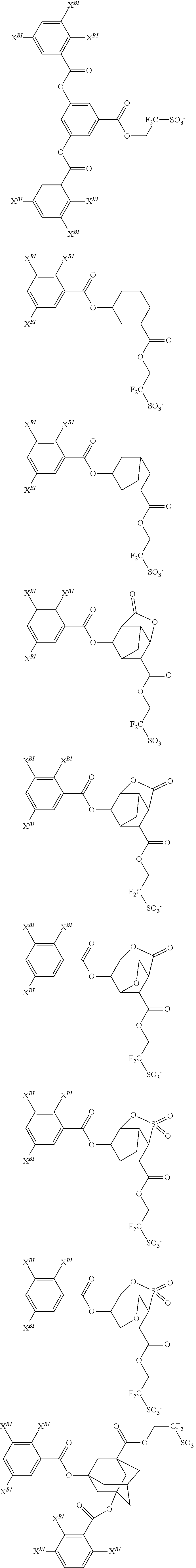

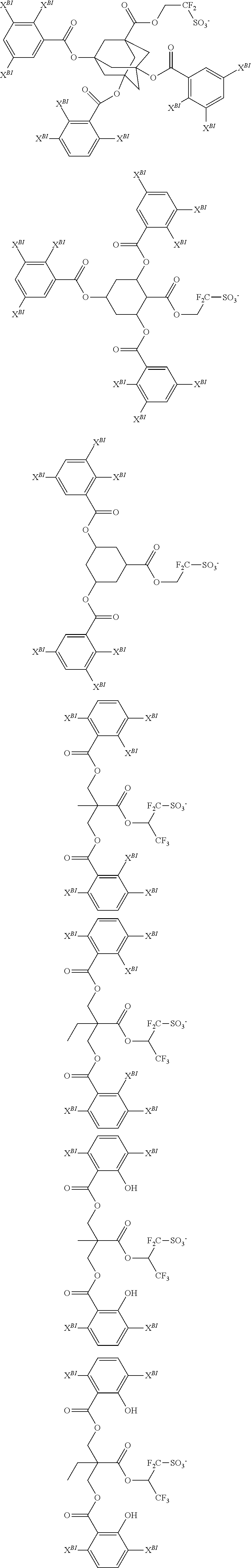

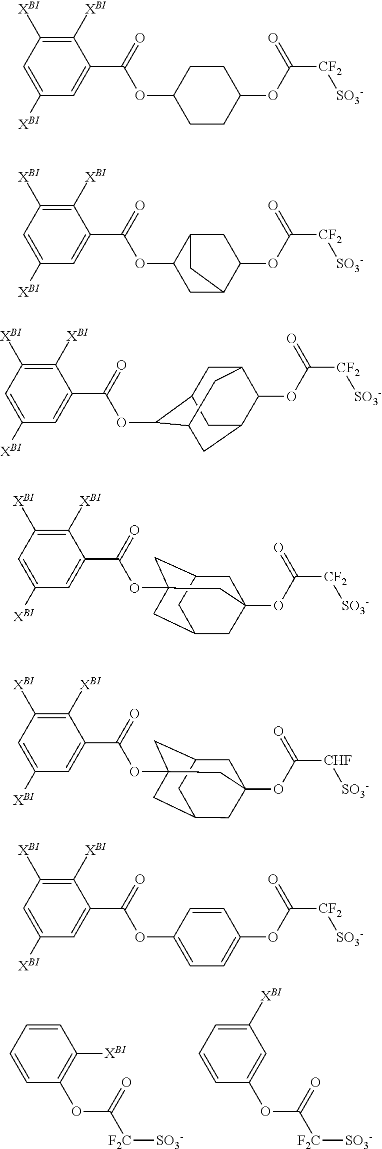

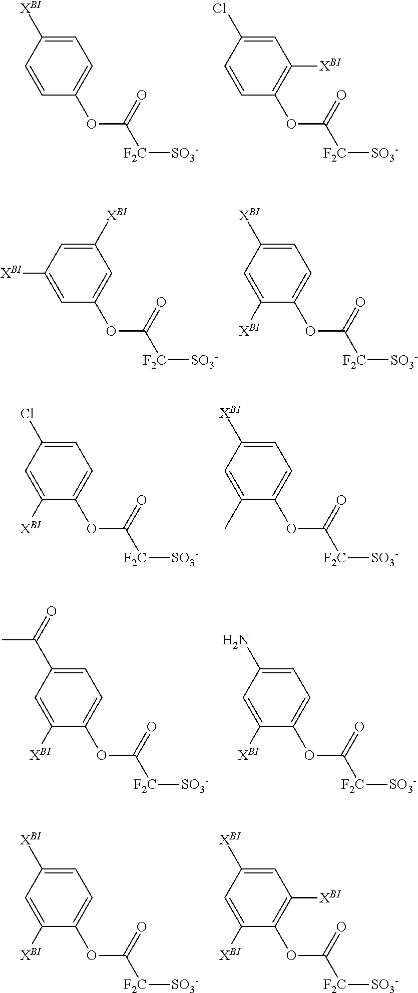

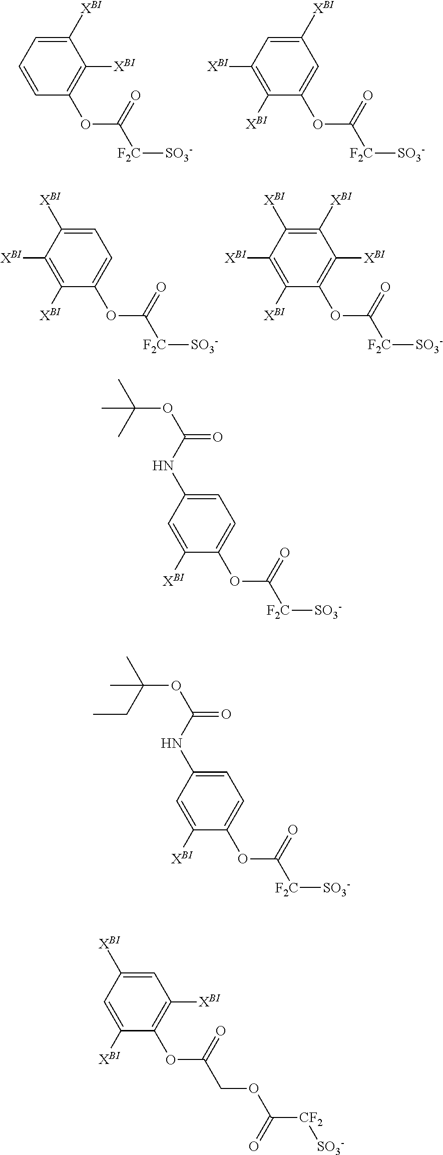

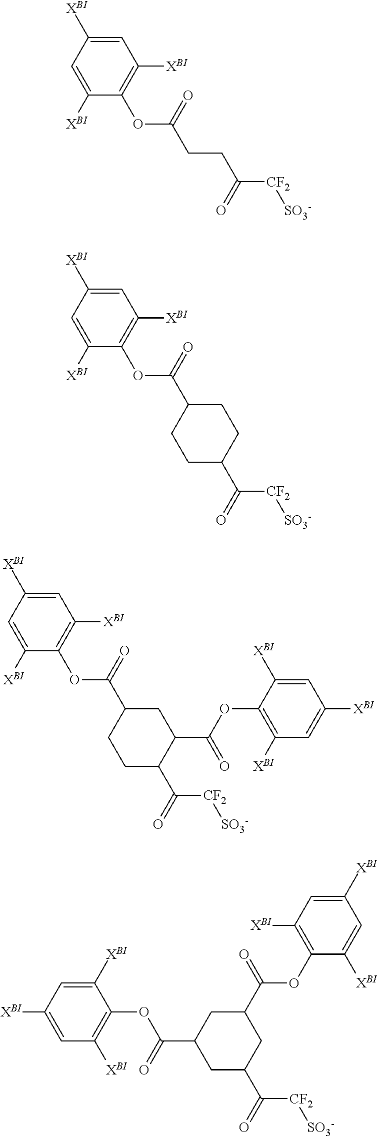

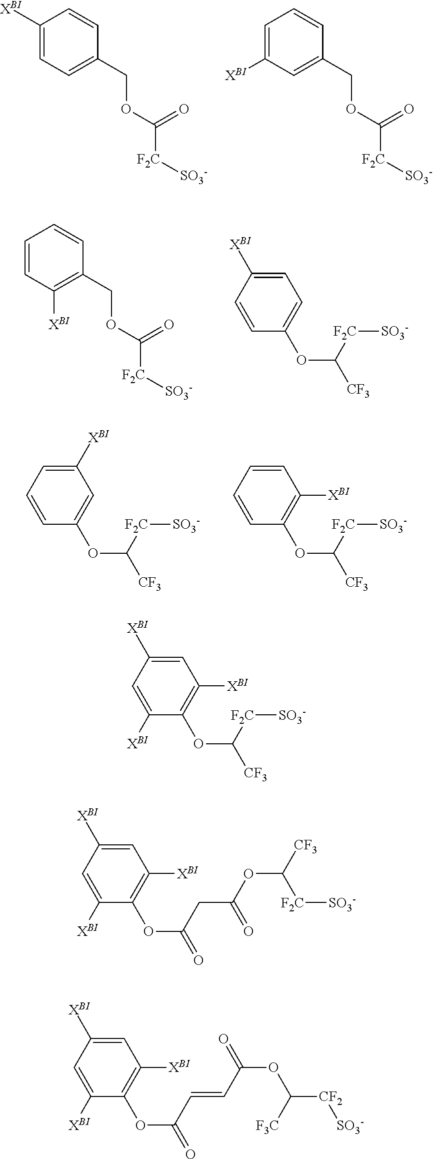

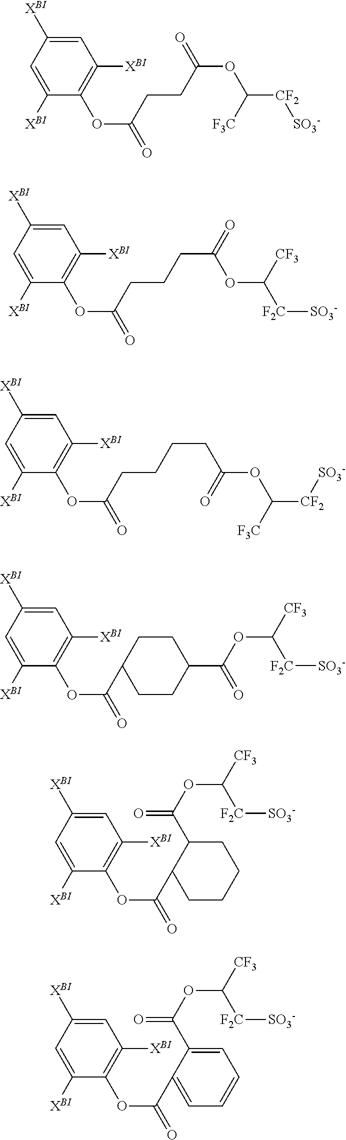

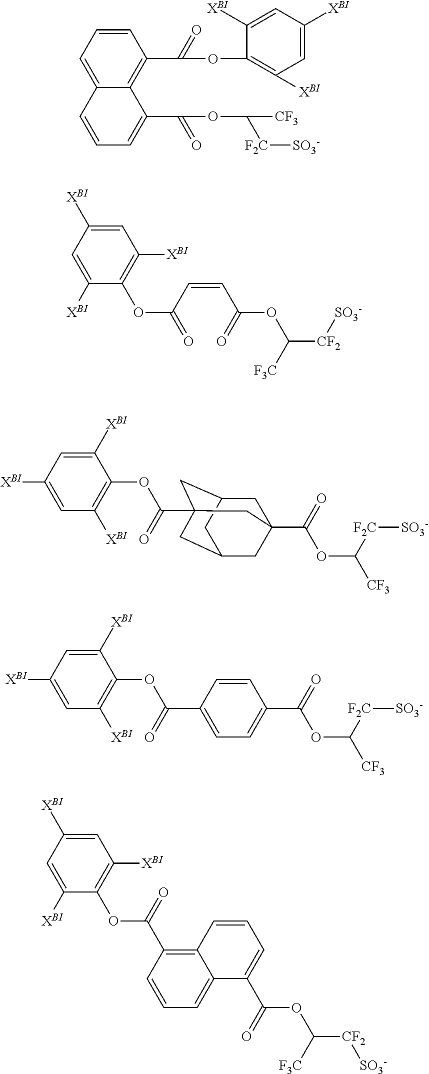

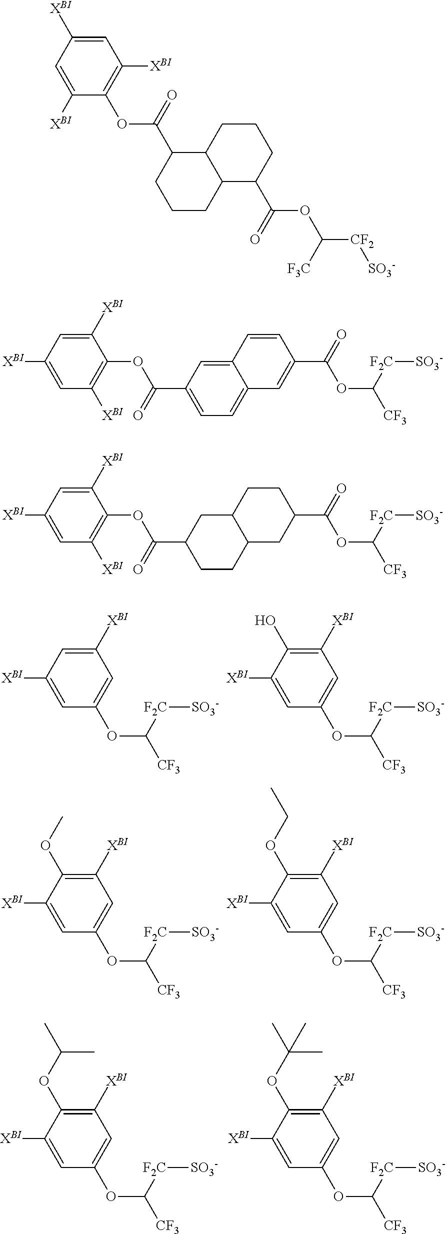

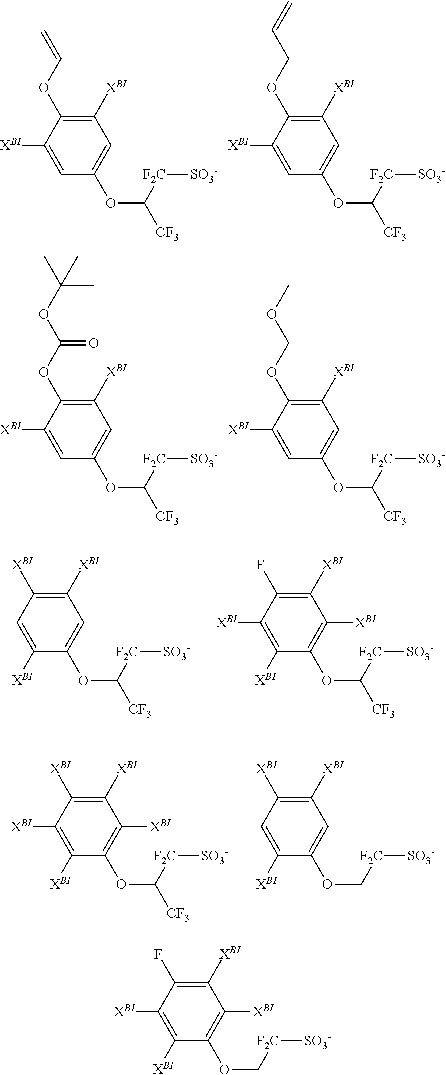

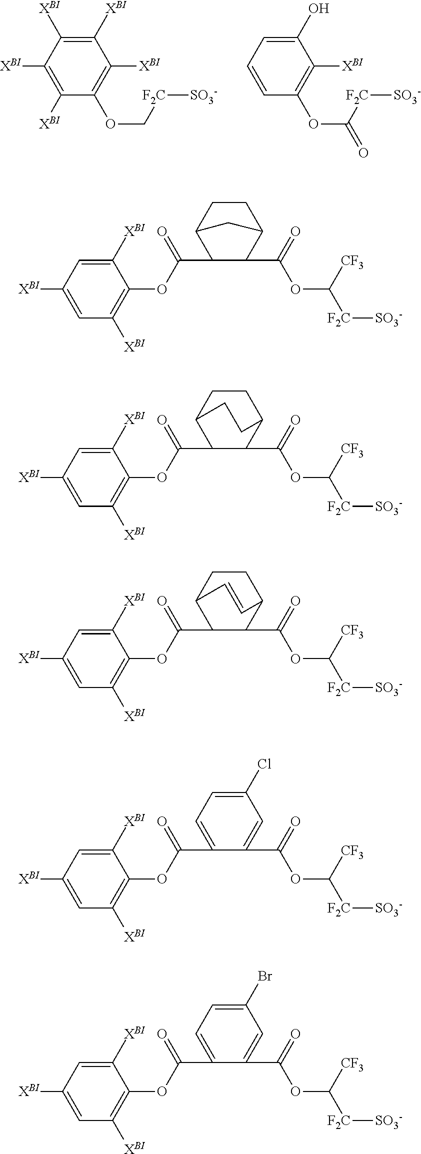

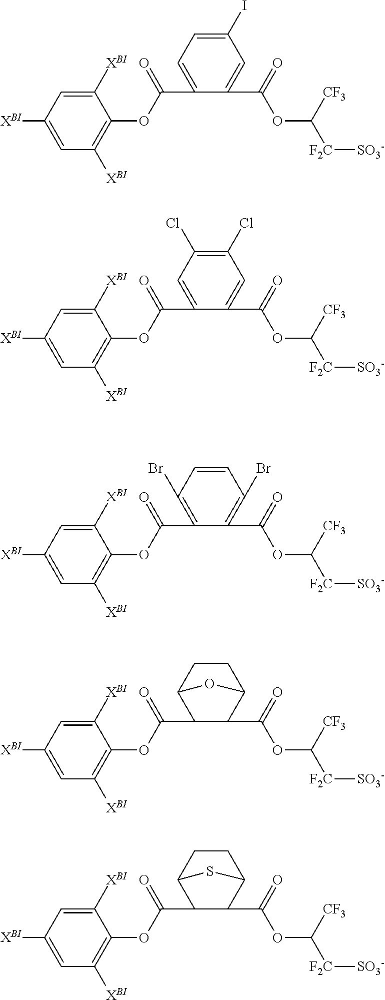

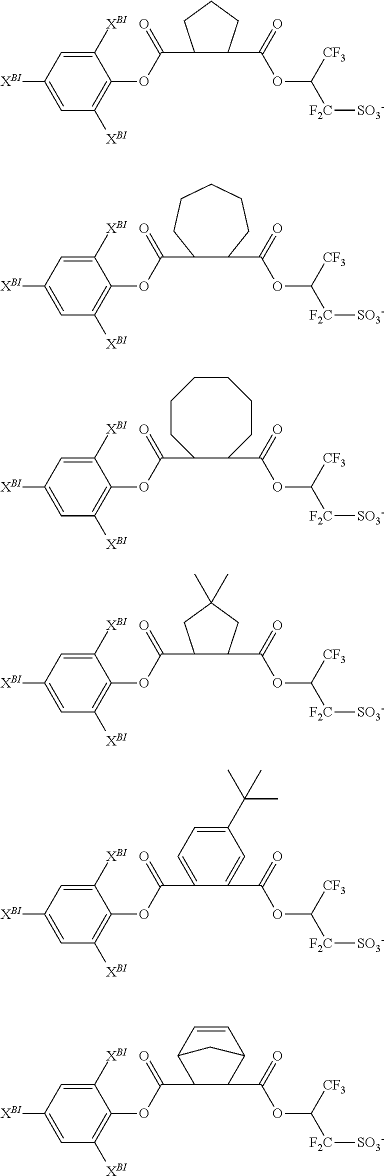

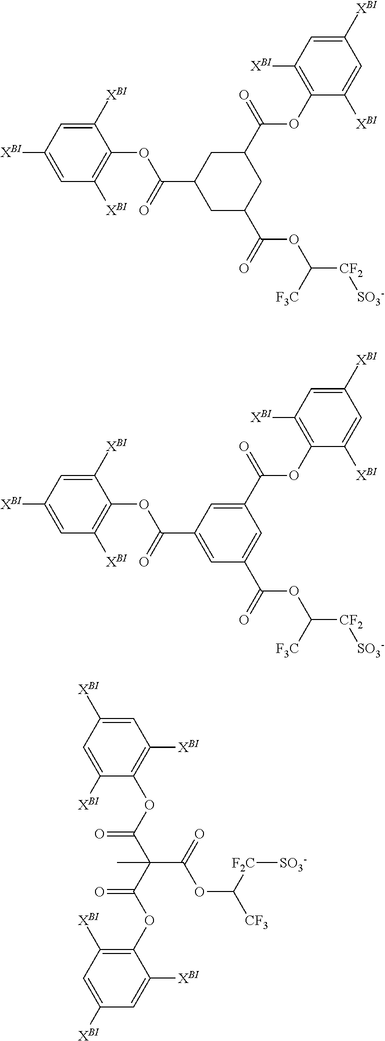

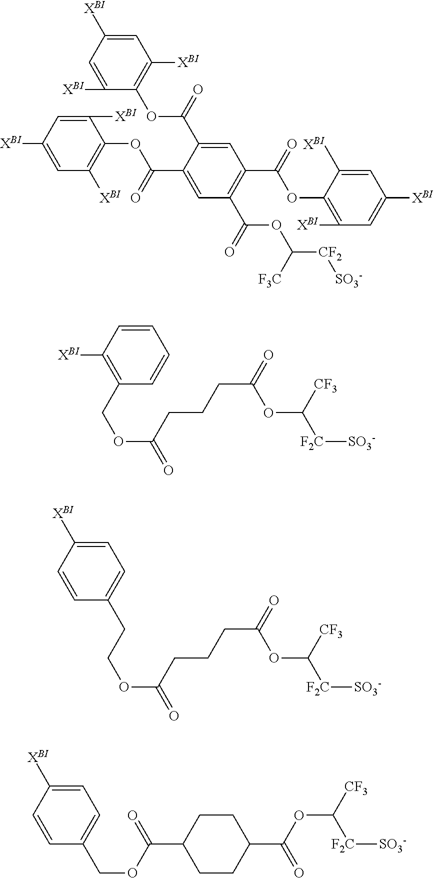

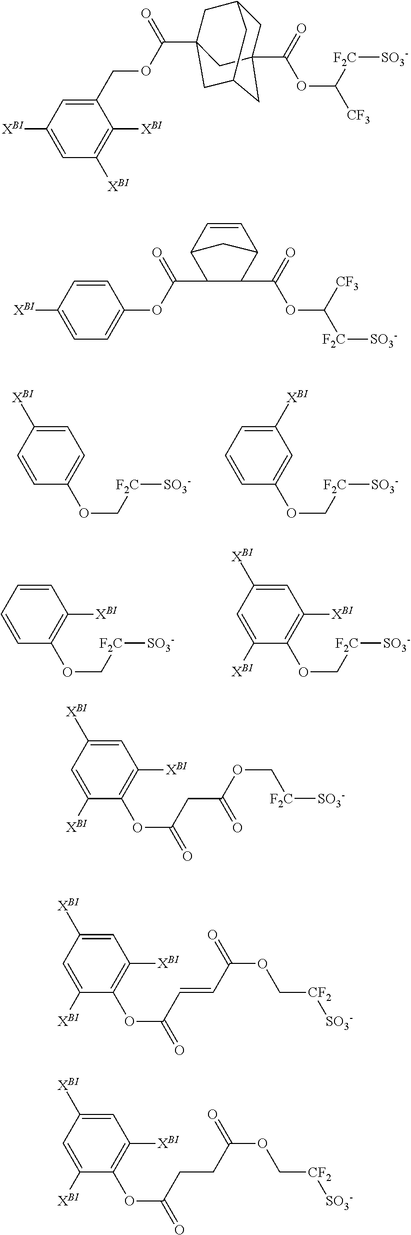

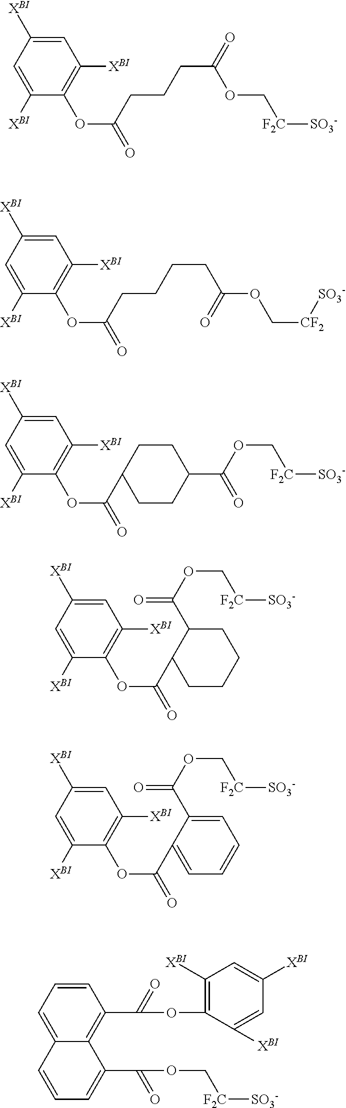

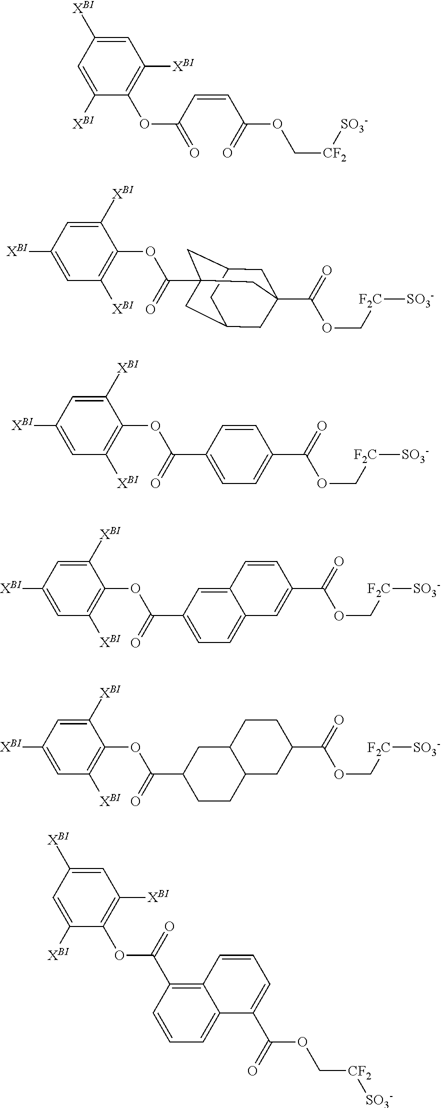

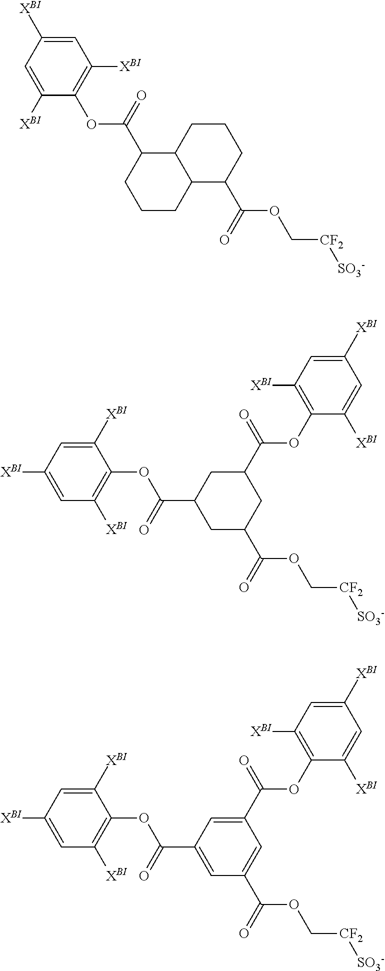

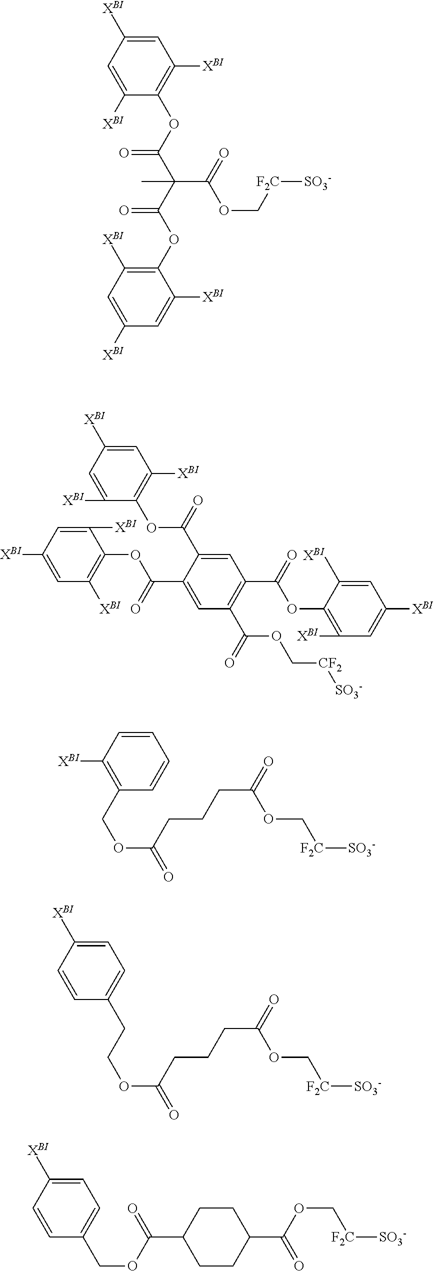

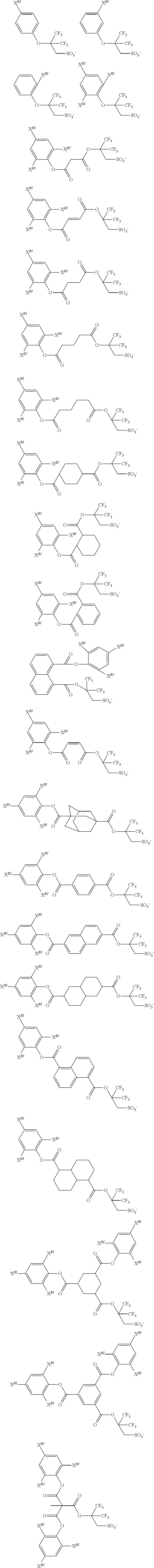

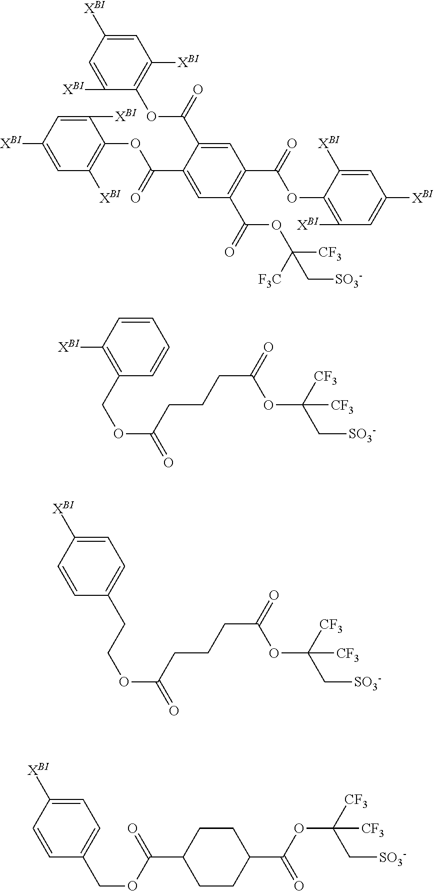

Other examples of the anion M⁻ include those described in the following patent documents.
WO 2023/157455, paragraphs [0076] and [01016]
WO 2024/24801, paragraph [0111]
WO 2024/43121, paragraphs [0253]-[0256]
WO 2024/57751, paragraphs [0044]-[0045]
WO 2024/122423, paragraphs [0205]-[0220]
JP-A 2023-123183, paragraphs [0170]-[0178]
JP-A 2024-062406, paragraphs [0026]-[0028]
JP-A 2024-062407, paragraphs [0022]-[0025]
JP-A 2024-062408, paragraphs [0026]-[0028]
JP-A 2024-068156, paragraphs [0028]-[0030]
JP-A 2024-068157, paragraphs [0026]-[0028]
JP-A 2024-068158, paragraphs [0028]-[0030]
JP-A 2024-068159, paragraphs [0028]-[0030]
JP-A 2024-072280, paragraphs [0031]-[0033]
JP-A 2024-072281, paragraphs [0023]-[0025]
JP-A 2024-077618, paragraphs [0026]-[0029]
JP-A 2024-077619, paragraphs [0020]-[0021]
JP-A 2024-080672, paragraphs [0140]-[0143]
JP-A 2024-083303, paragraphs [0023]-[0025]
JP-A 2024-083304, paragraphs [0028]-[0031]
JP-A 2024-099500, paragraphs [0030]-[0033]
JP-A 2024-099502, paragraphs [0028]-[0030]
JP-A 2024-101557, paragraphs [0030]-[0032]
JP-A 2024-102842, paragraphs [0025]-[0027]
JP-A 2024-102843, paragraphs [0033]-[0035]
JP-A 2024-127832, paragraphs [0021]-[0022]
JP-A 2024-144354, paragraphs [0169]-[0172]
JP-A 2024-144356, paragraphs [0178]-[0181]
JP-A 2024-160436, paragraphs [0040]-[0143]
JP 7247732, paragraphs [0157]-[0158]
JP 7446352, paragraphs [0227]-[0238]
JP 7466597, paragraphs [0253]-[0256]
JP 7466782, paragraphs [0309]-[0312]

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, JP-A 2019-202974, and JP-A 2024-104830; fluorine-free benzenesulfonic acid or alkylsulfonic acid anions having an iodized aromatic group bonded thereto as described in JP 6645464. The anions described in JP-A 2024-077330, paragraphs [0229]-[0231] and JP-A 2024-140135, paragraphs [0033]-[0093] are also useful.

Also useful are the bissulfonic acid anions described in JP-A 2015-206932, the sulfonamide or sulfonimide anions having sulfonic acid side and different side, described in WO 2020/158366, and the anions having a sulfonic acid side and a carboxylic acid side, described in JP-A 2015-024989.

In formulae (c2) and (c3), d1 and d2 are each independently 0, 1, 2 or 3, preferably 1.

In formula (c4), e1 is 0 or 1, e2 is 0, 1, 2, 3 or 4, and e3 is 0, 1, 2, 3 or 4, meeting 0≤e2+e3≤4 when e1=0, and 0≤e2+e3≤6 when e1=1.

In formulae (c2), (c3) and (c4), L¹ is a single bond, ether bond, ester bond, carbonyl, sulfonate ester bond, sulfonamide bond, carbonate bond or carbamate bond. From the aspect of synthesis, an ether bond, ester bond or carbonyl is preferred, with the ester bond or carbonyl being more preferred.

In formula (c2), Rf¹ and Rf² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine or a C₁-C₆ fluorinated saturated hydrocarbyl group. It is preferred for solvent solubility that at least one of Rf³ and Rf⁴ be trifluoromethyl.

In formula (c3), Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In formula (c4), Rf⁷ is fluorine, a C₁-C₆ fluorinated alkyl group, C₁-C₆ fluorinated alkoxy group, or C₁-C₆ fluorinated alkylthio group. Rf⁷ is preferably fluorine, trifluoromethyl, difluoromethyl, trifluoromethoxy, difluoromethoxy, trifluoromethylthio or difluoromethylthio, more preferably fluorine, trifluoromethyl or trifluoromethoxy. When e2 is 2, 3 or 4, a plurality of Rf⁷ may be identical or different.

In formula (c4), R⁴³ is halogen exclusive of fluorine, or a C₁-C₂₀ hydrocarbyl group which may contain a heteroatom. The hydrocarbyl group may be saturated or unsaturated and straight, branched or cyclic. Examples thereof are as exemplified above for the hydrocarbyl group R¹ to R³ in formula (1), but not limited thereto. When e3 is 2, 3 or 4, a plurality of R⁴³ may be identical or different.

When e3 is 2, 3 or 4, a plurality of R⁴³ may bond together to form a ring with the carbon atoms to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

Examples of the anion in repeat unit (c2) are shown below, but not limited thereto. R^{A} is as defined above.

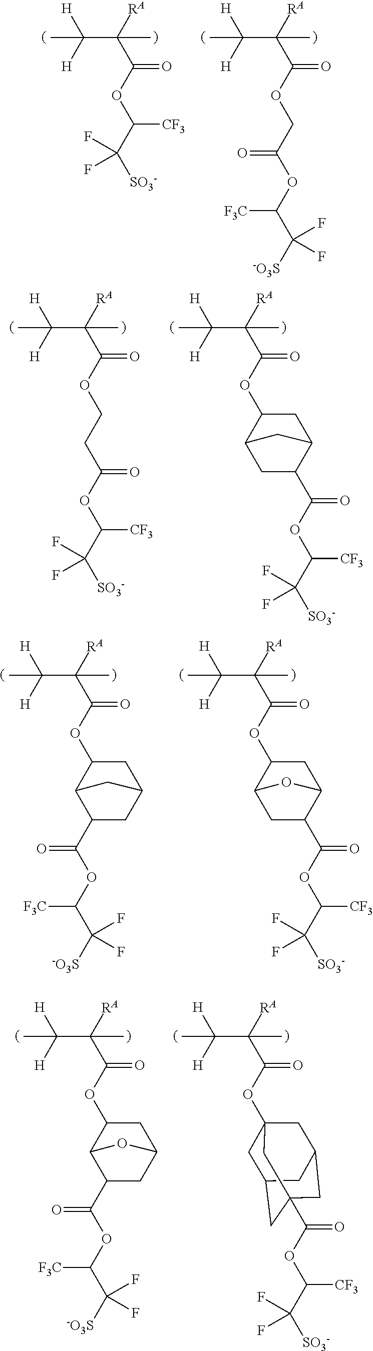

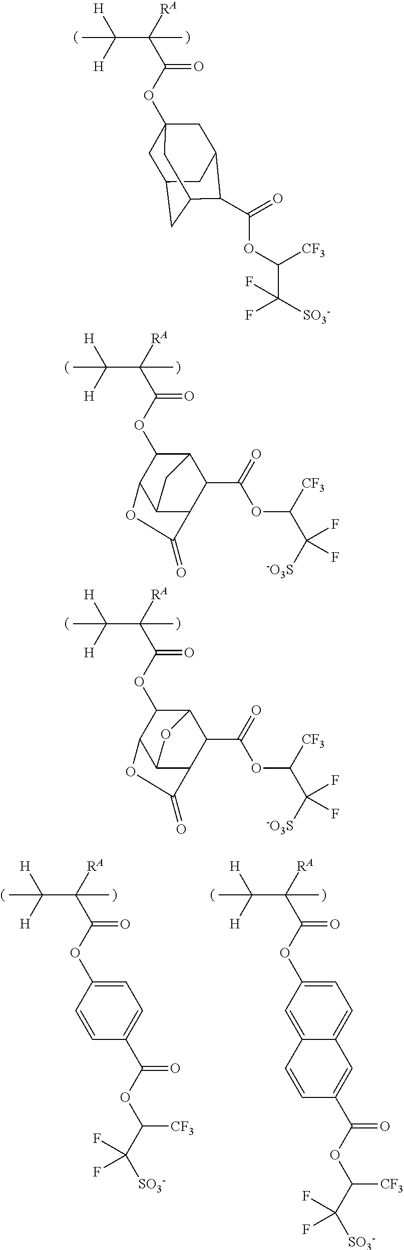

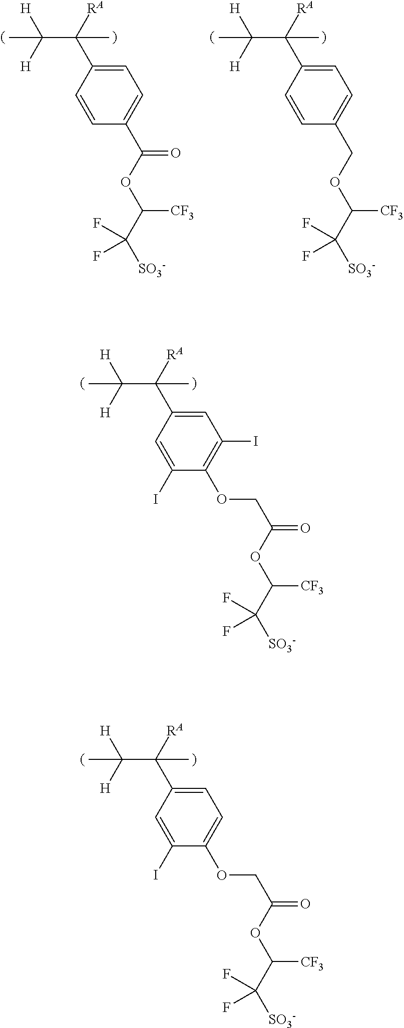

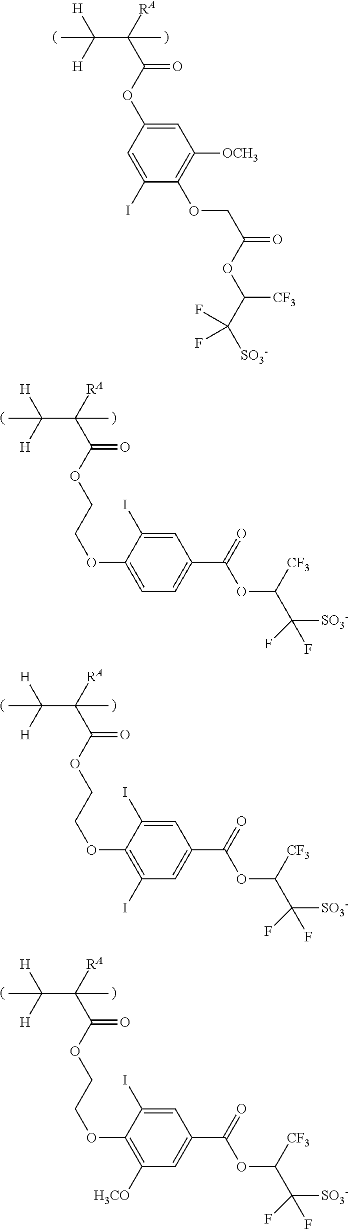

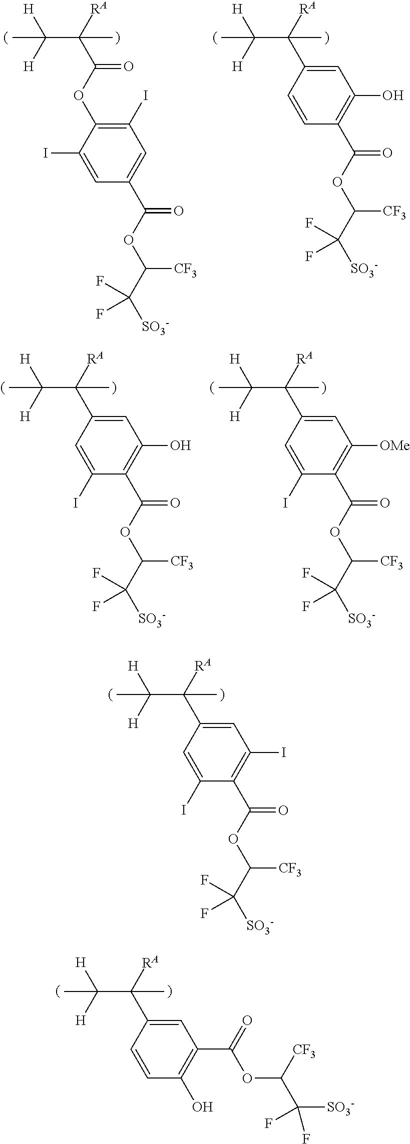

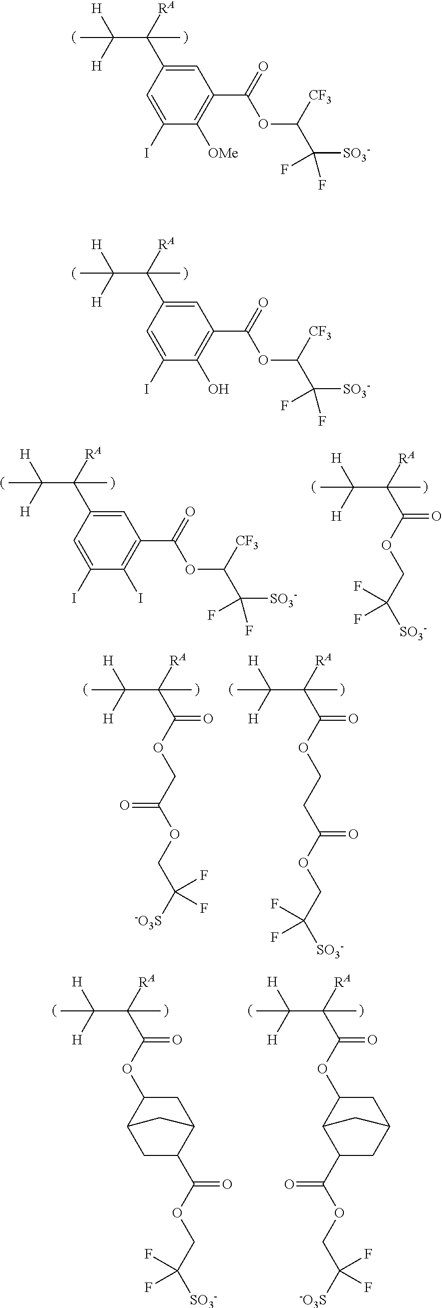

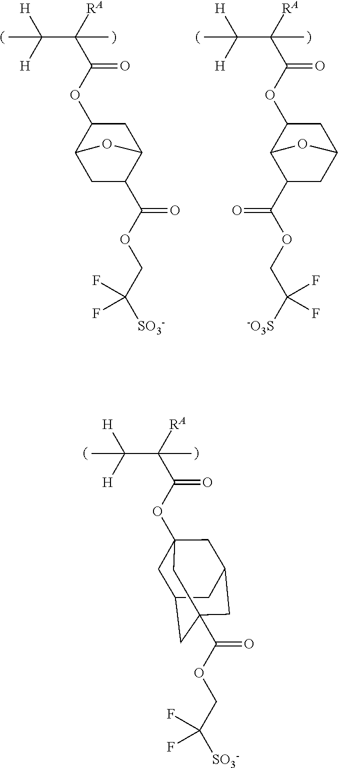

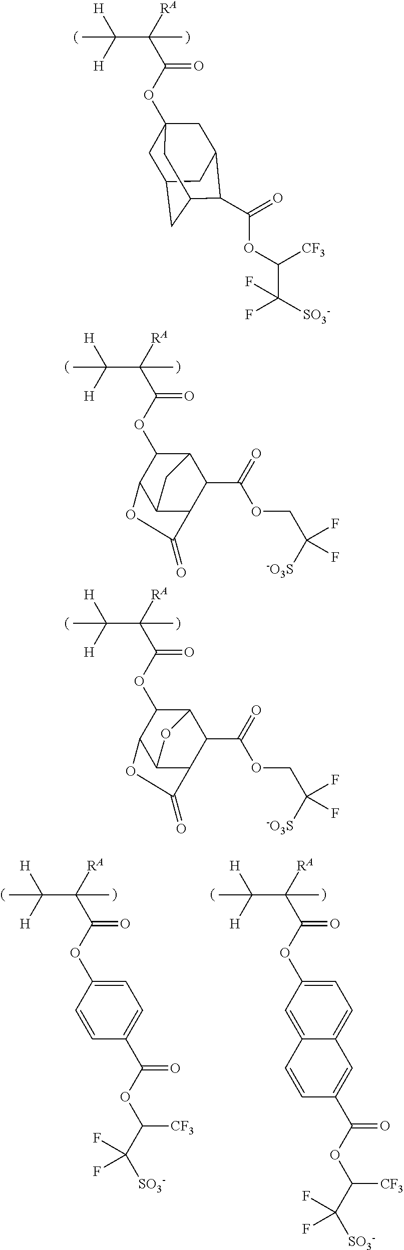

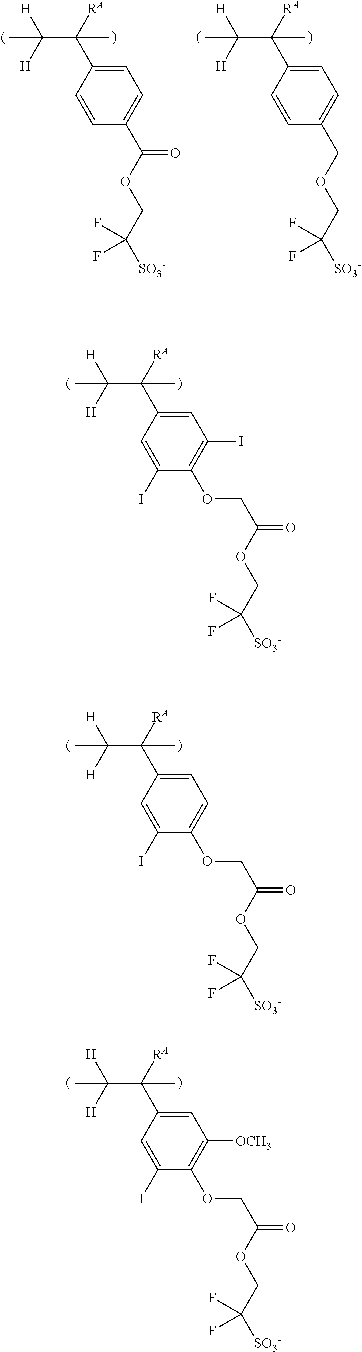

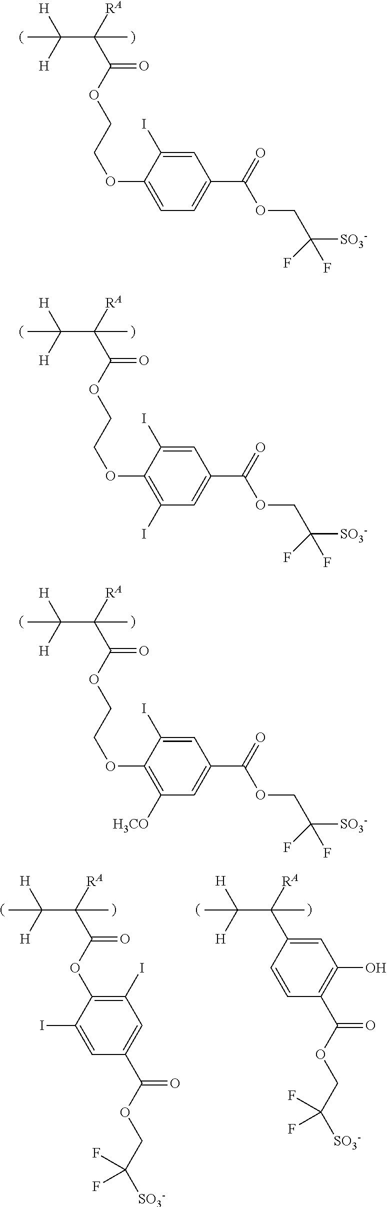

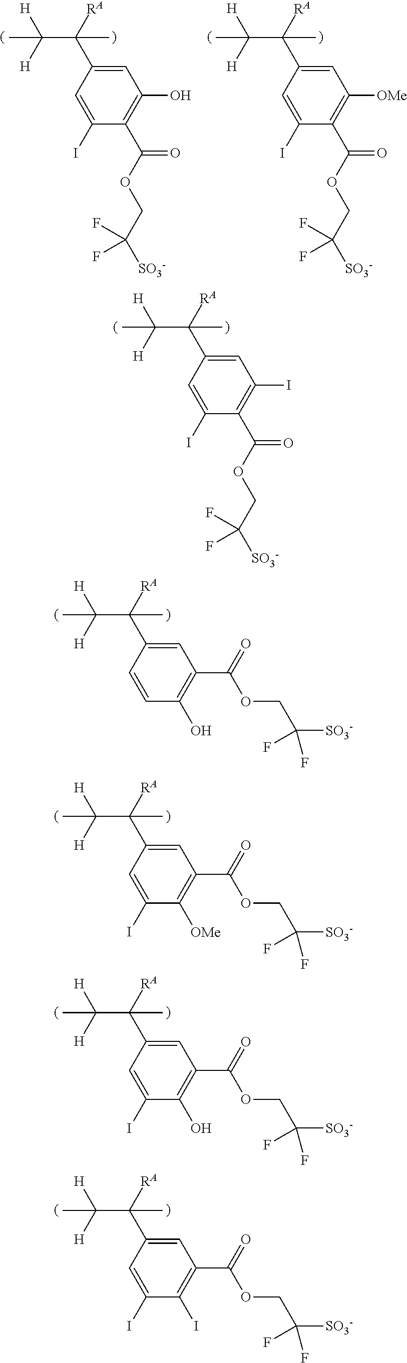

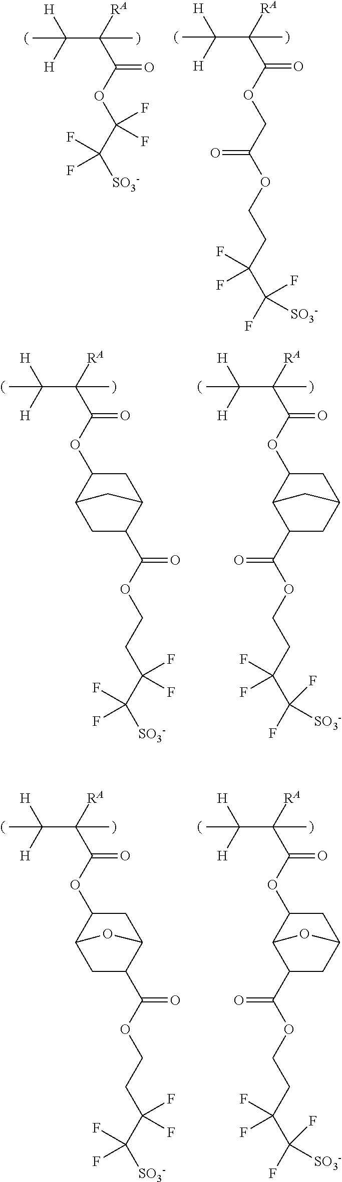

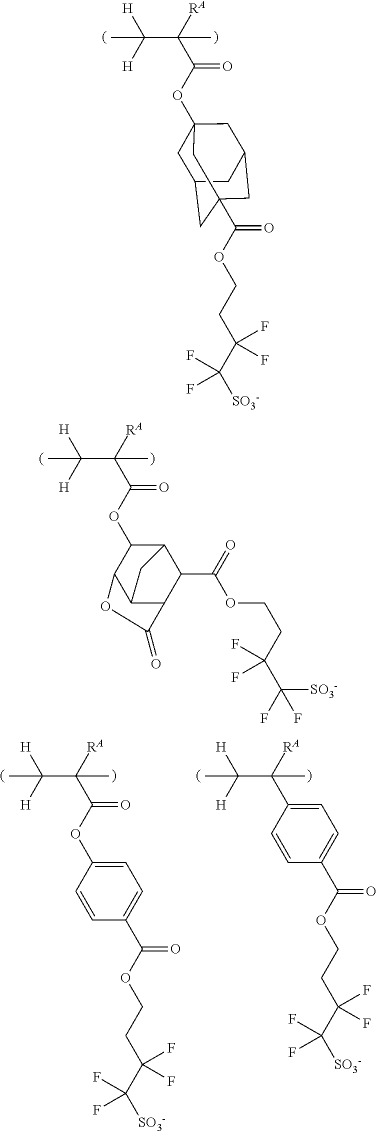

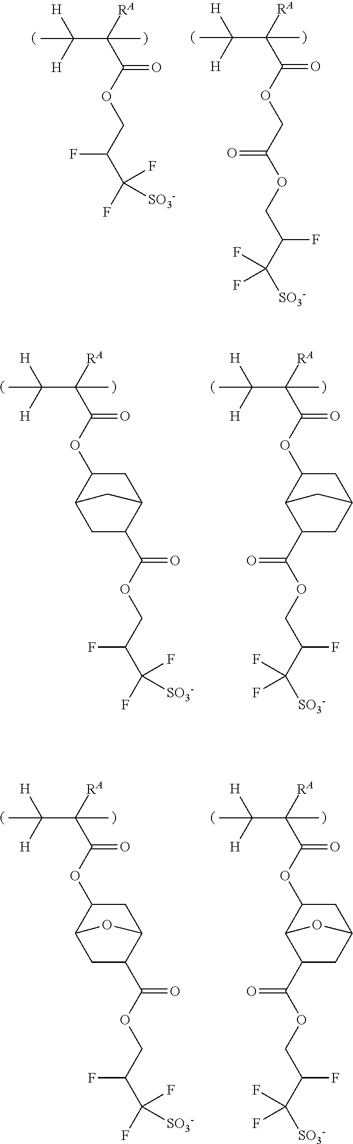

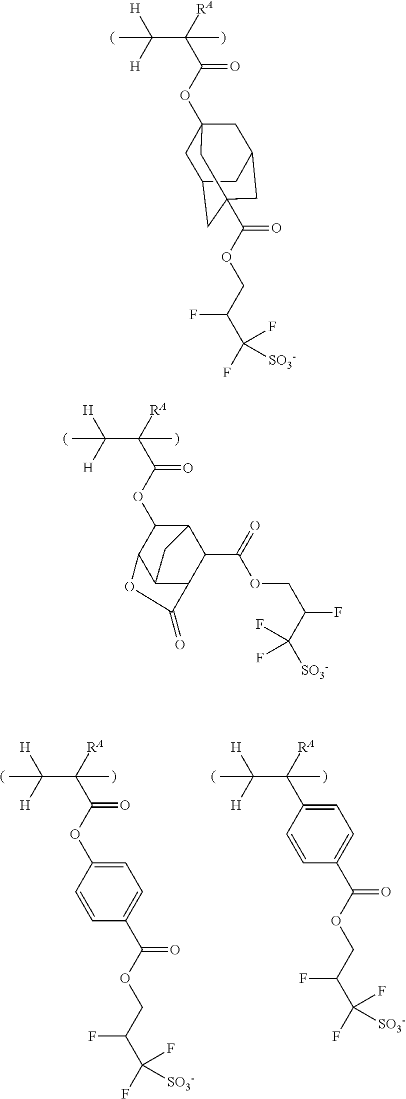

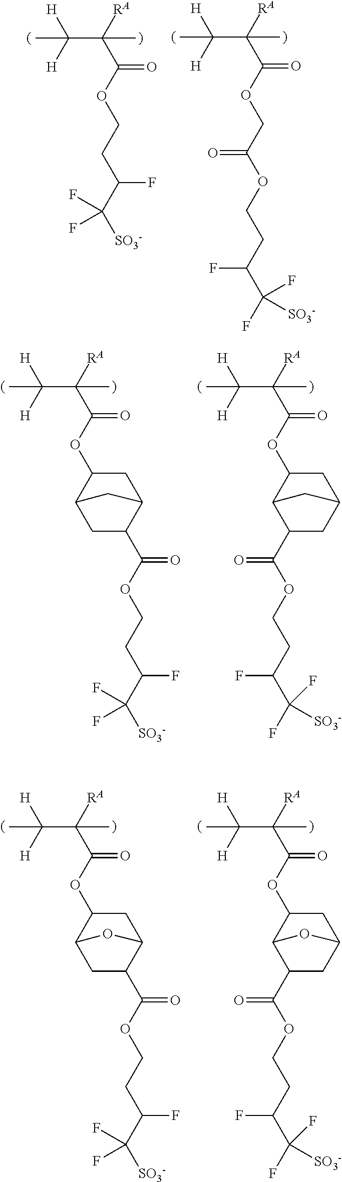

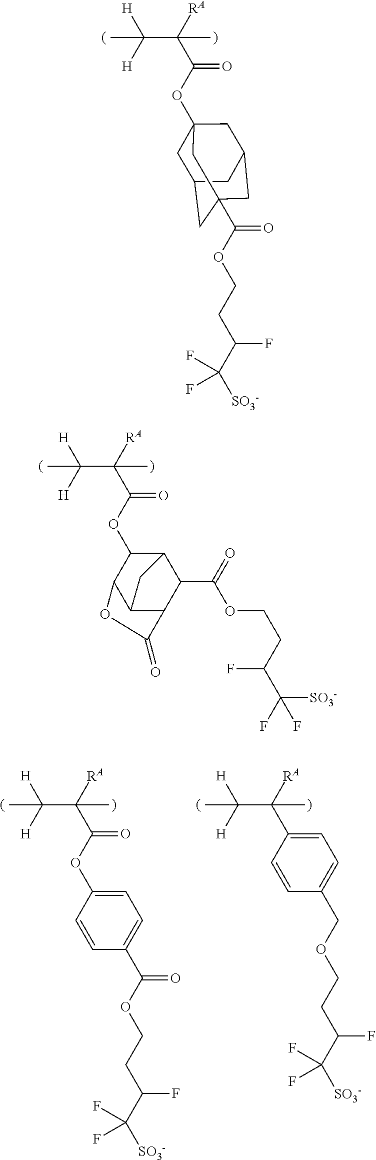

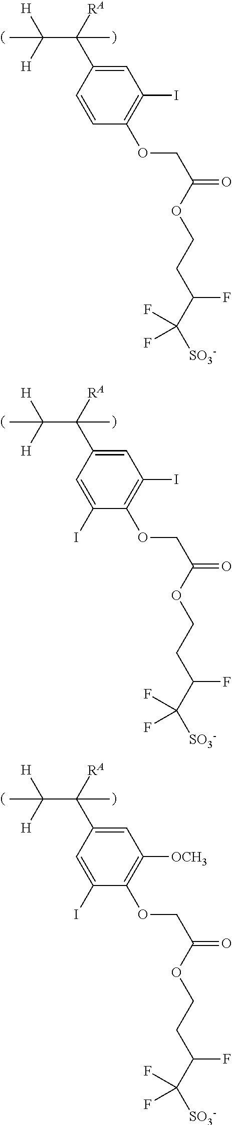

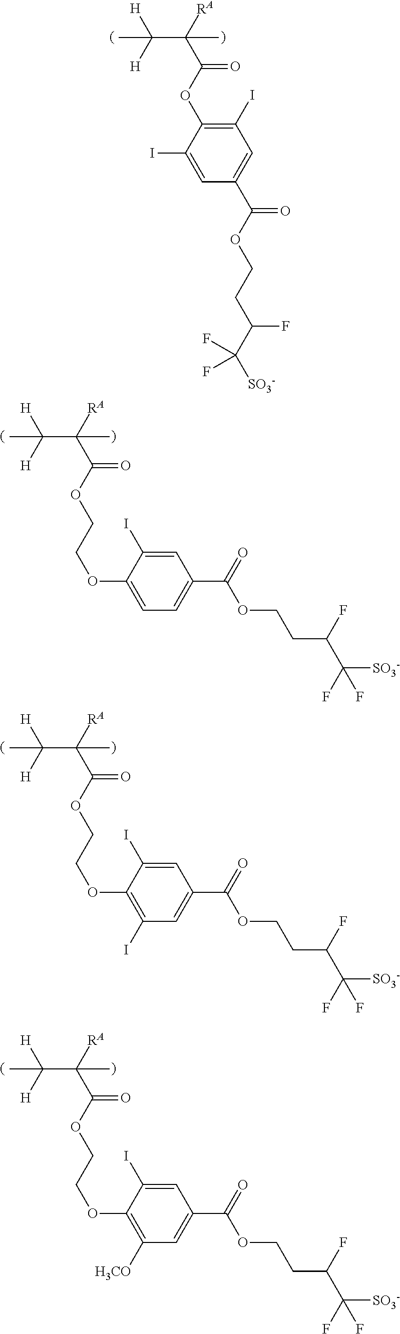

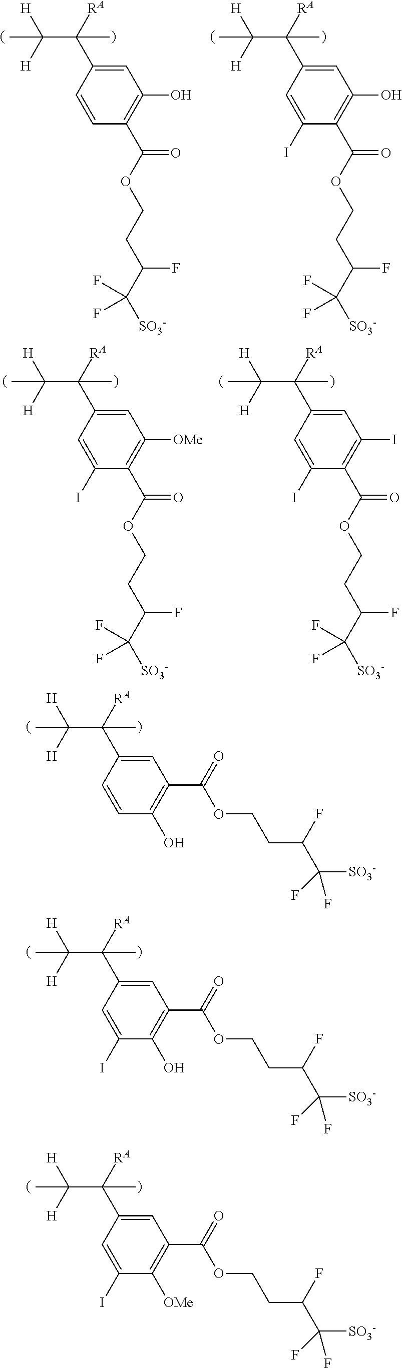

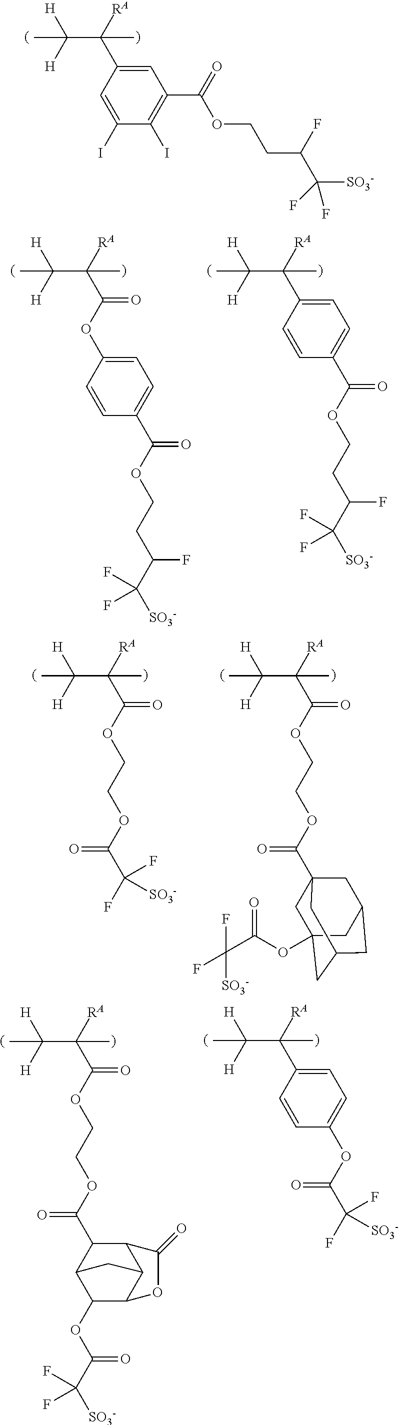

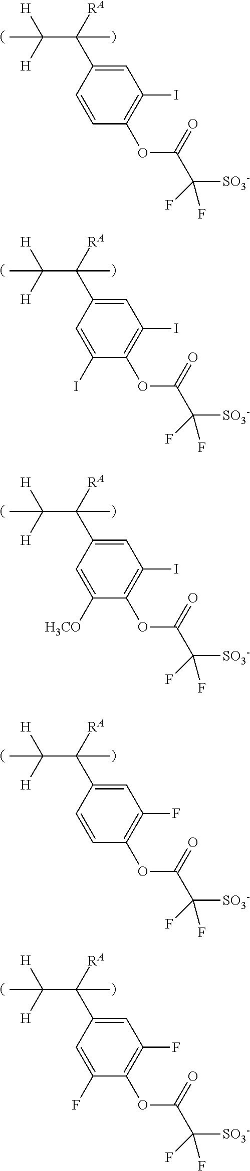

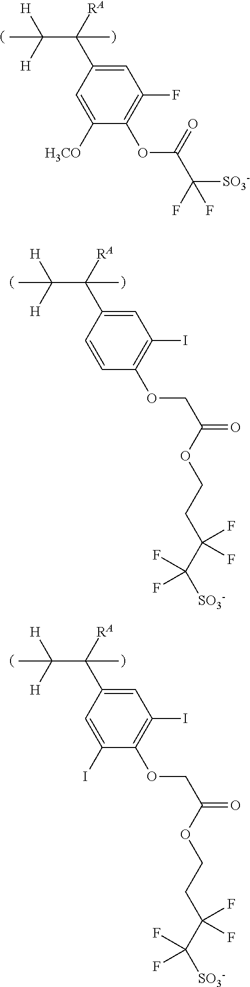

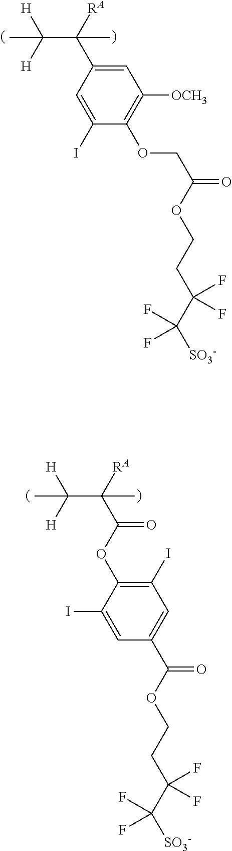

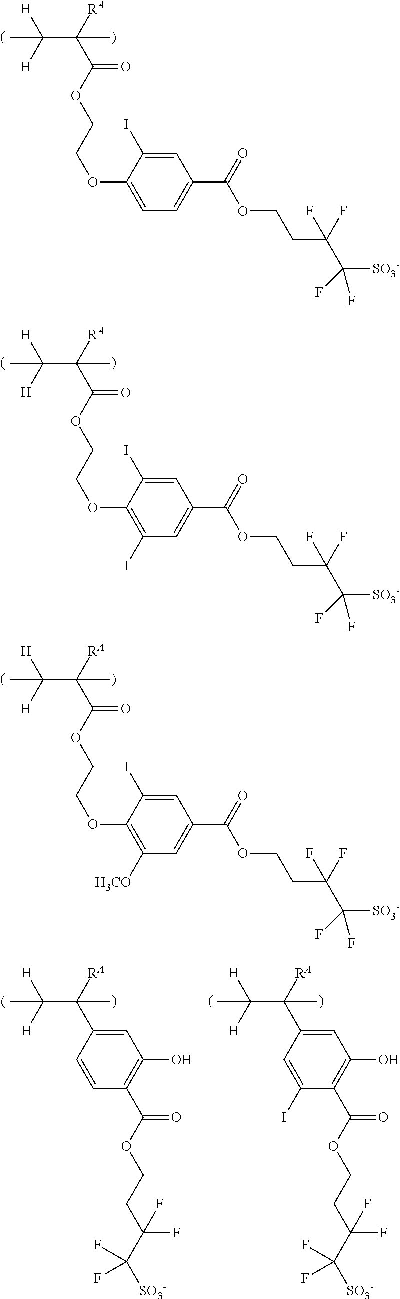

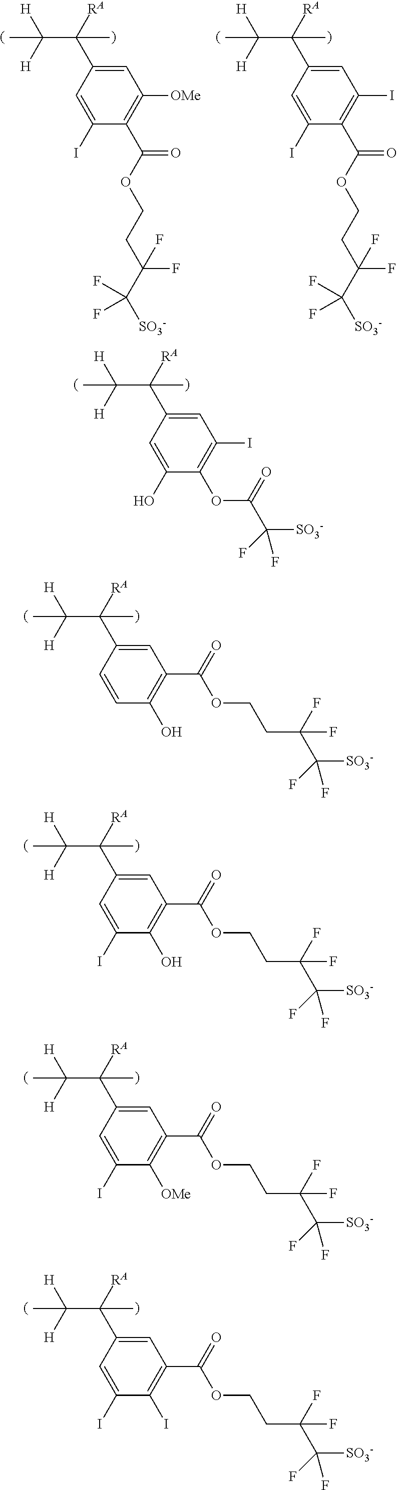

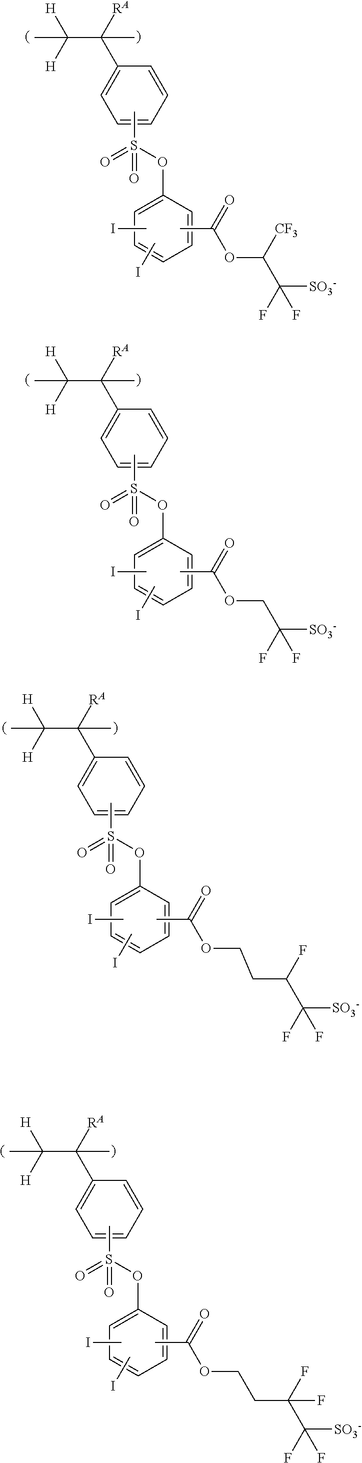

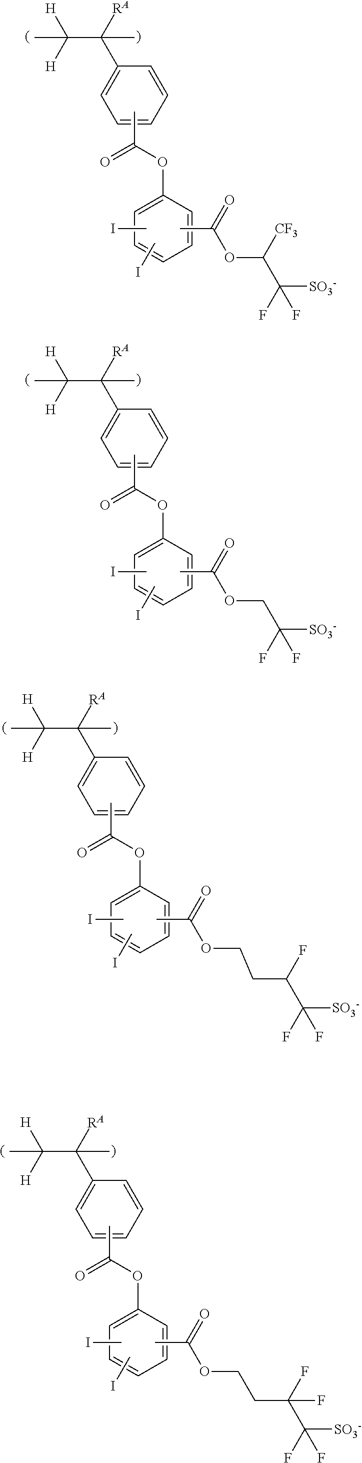

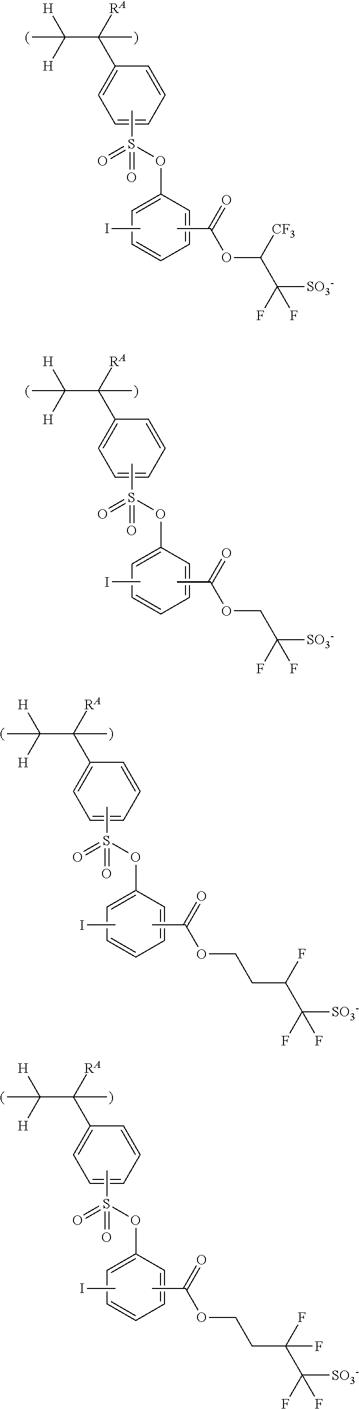

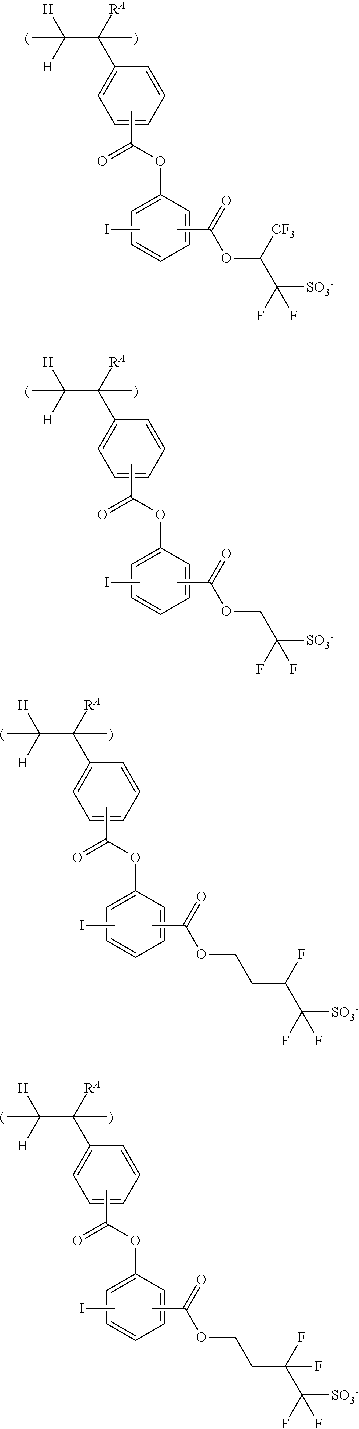

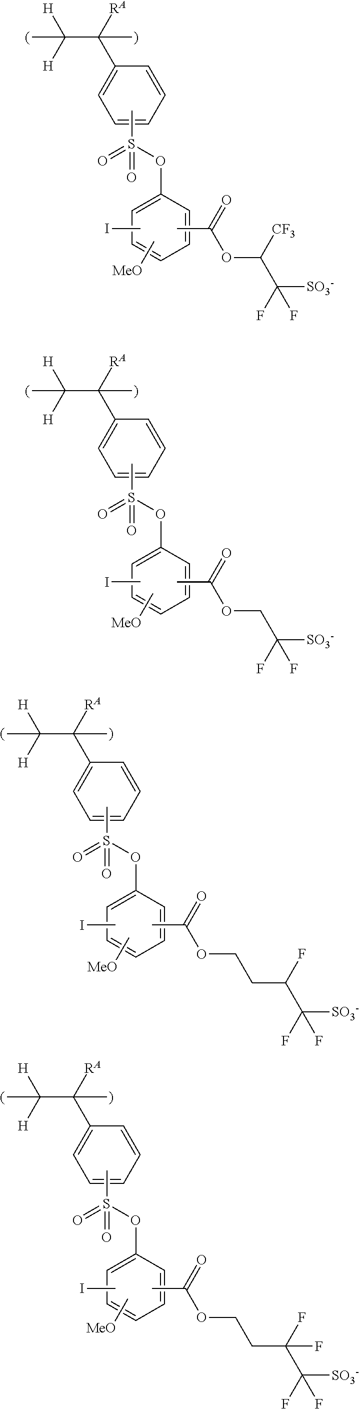

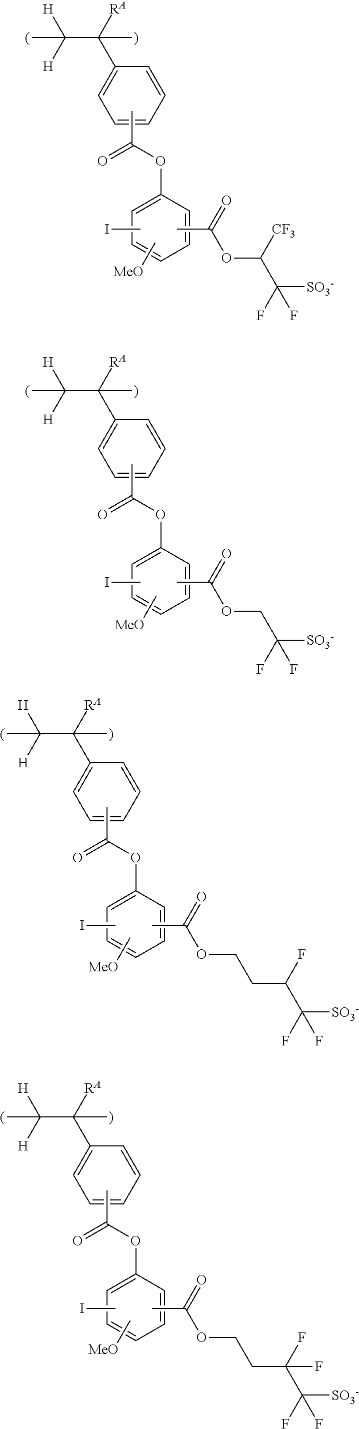

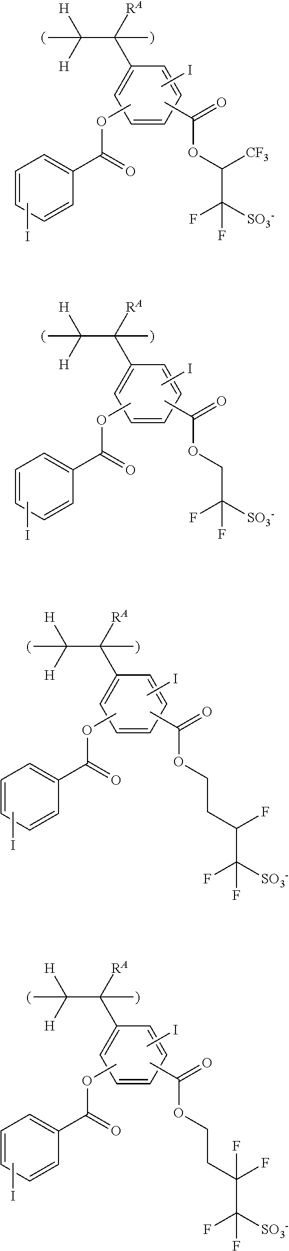

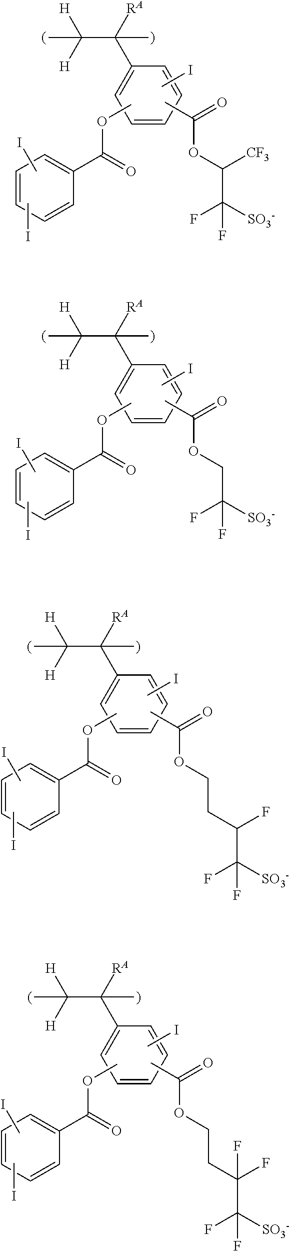

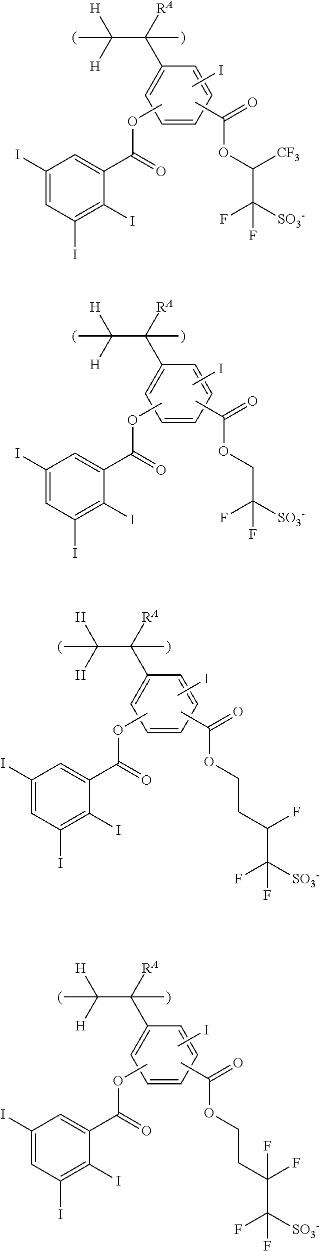

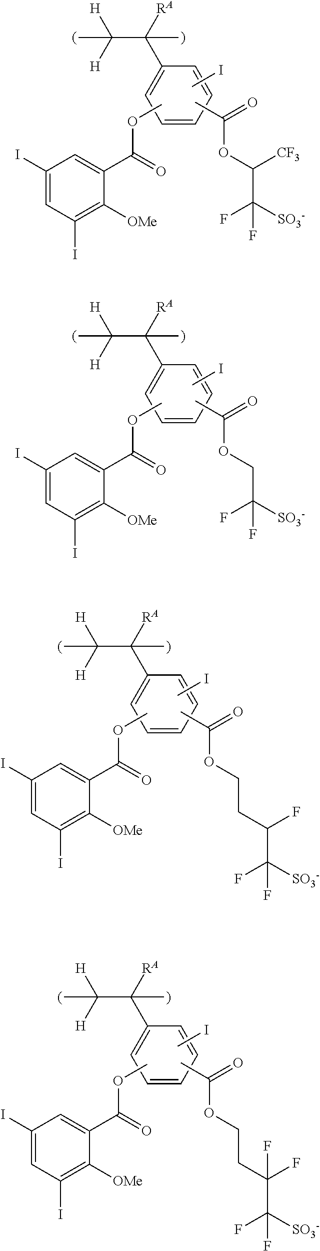

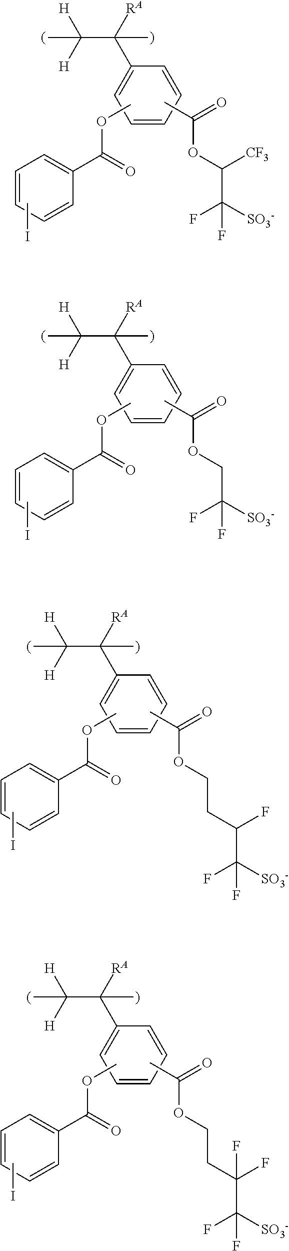

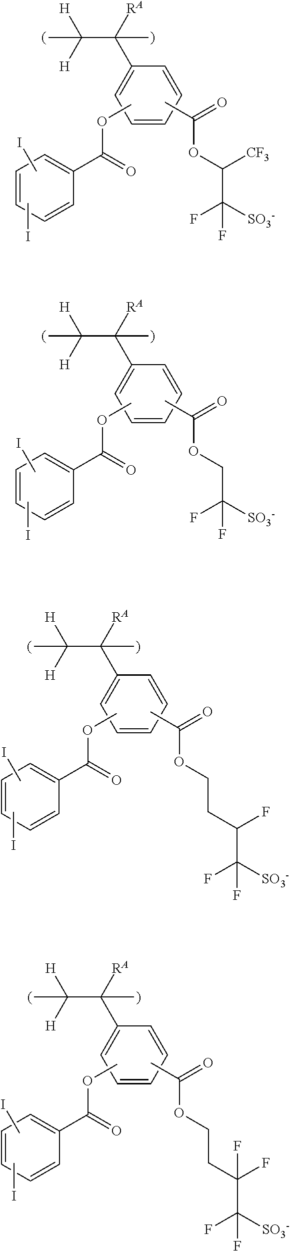

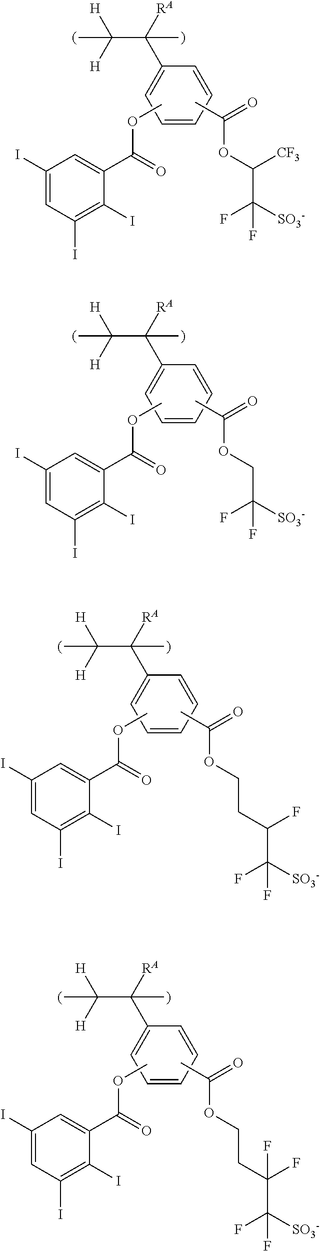

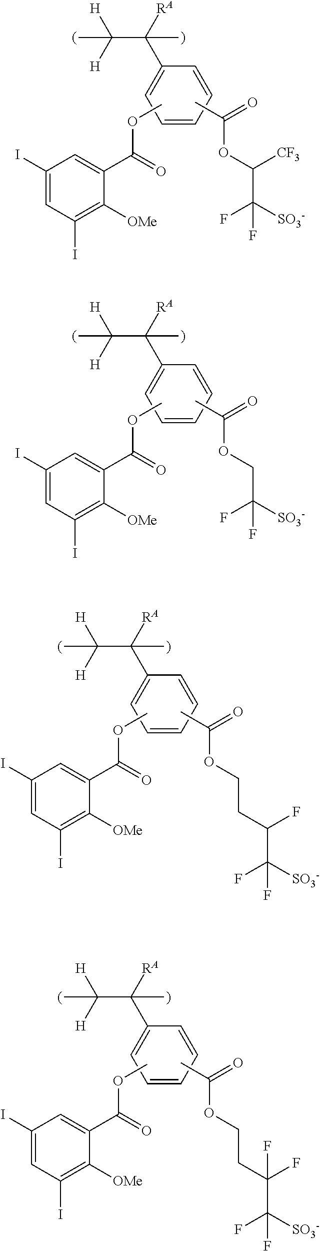

Other examples of the repeat unit (c2) include those described in the following patent documents.
WO 2024/176672, paragraphs [0043]-[0044]
WO 2024/176701, paragraphs [0067]-[0070]
WO 2024/190386, paragraphs [0057]-[0058]
JP-A 2024-103465, paragraphs [0020]-[0023]
JP-A 2024/120703, paragraphs [0067]-[0070]
JP 7520258, paragraphs [0075]-[0084]

Examples of the anion in repeat unit (c3) are shown below, but not limited thereto. R^{A} is as defined above.

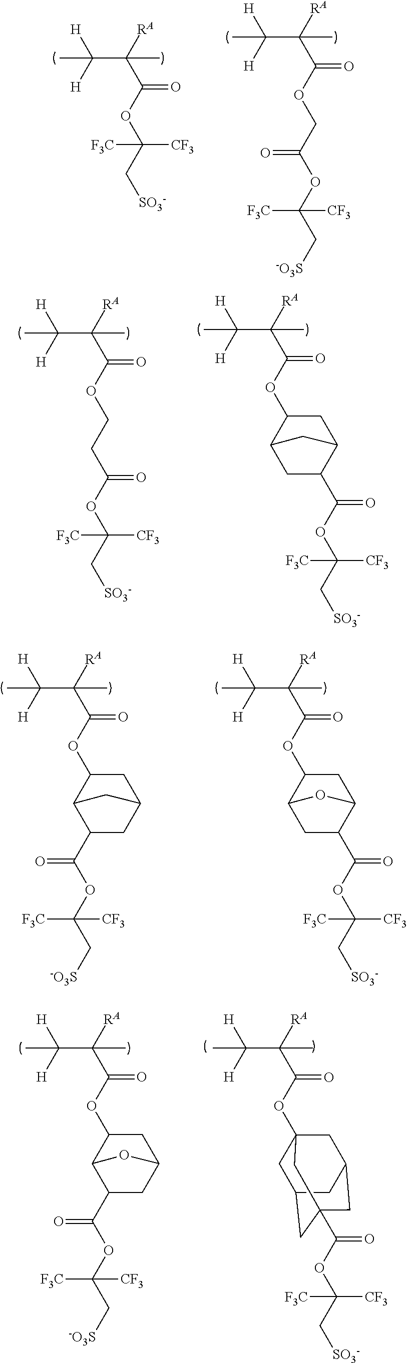

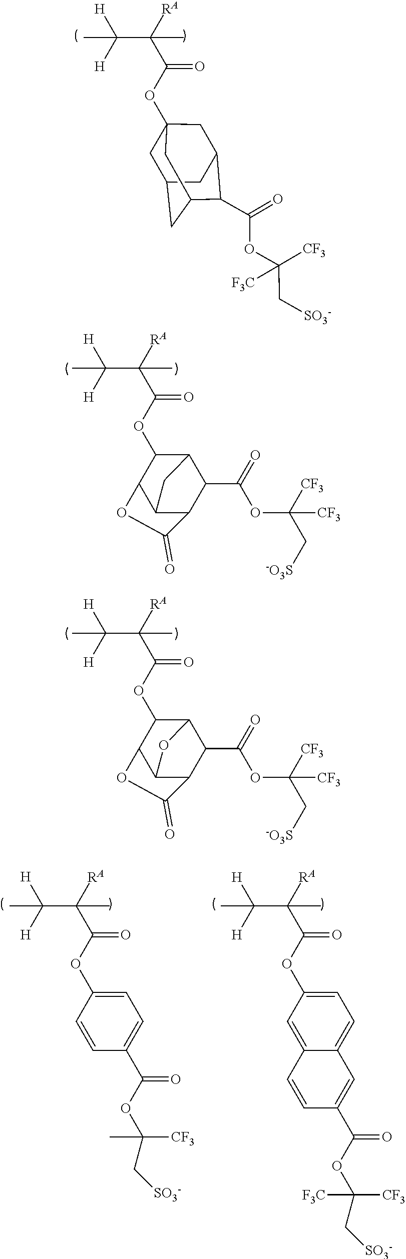

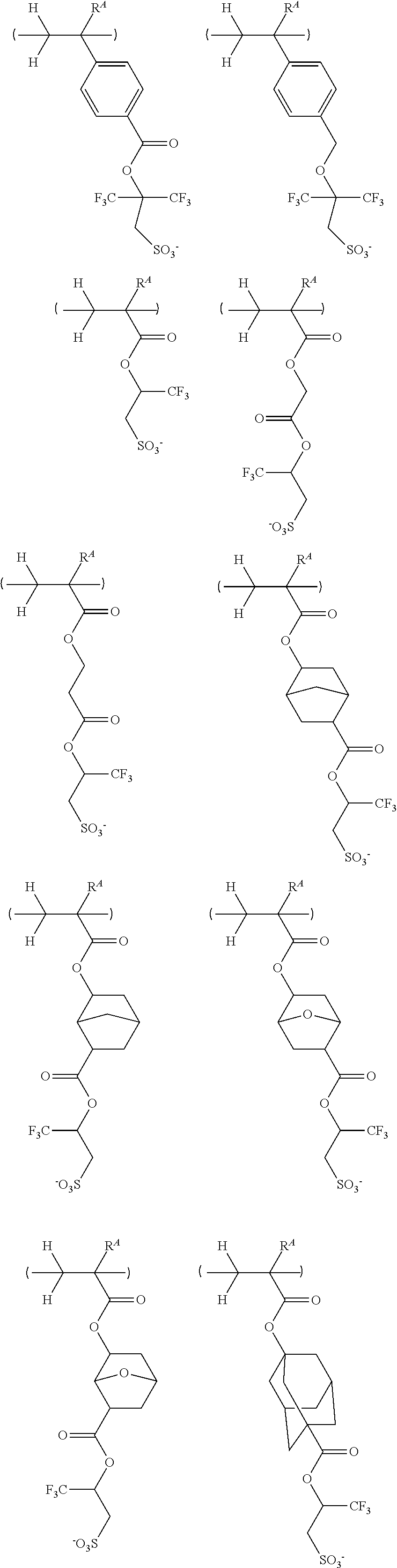

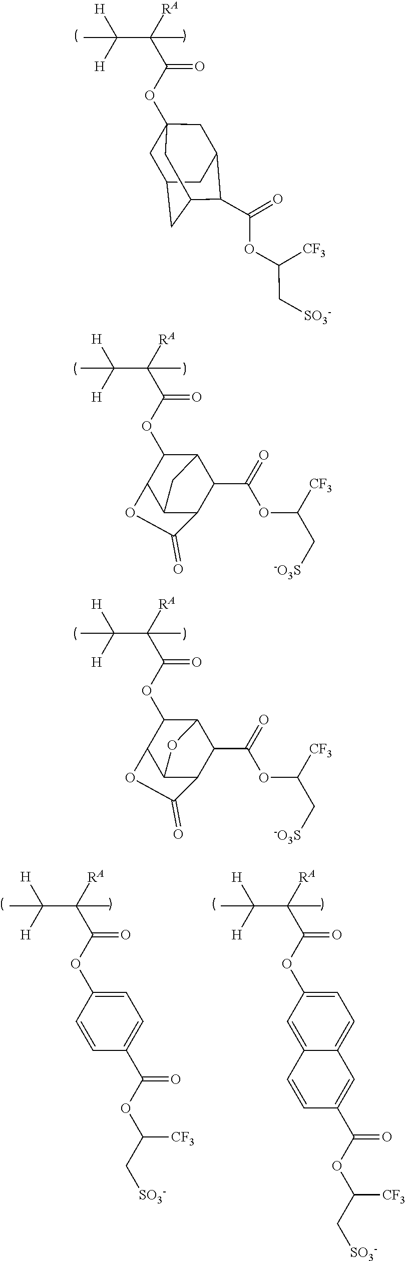

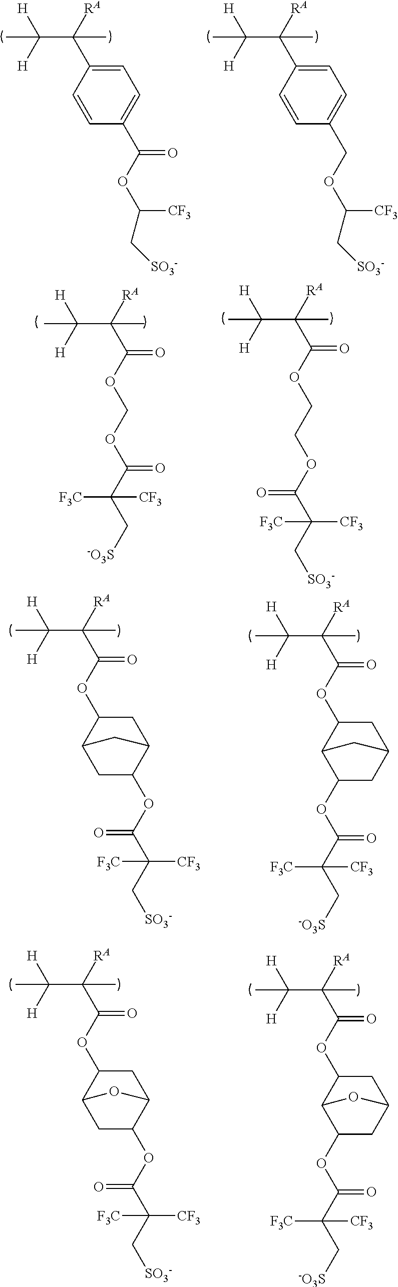

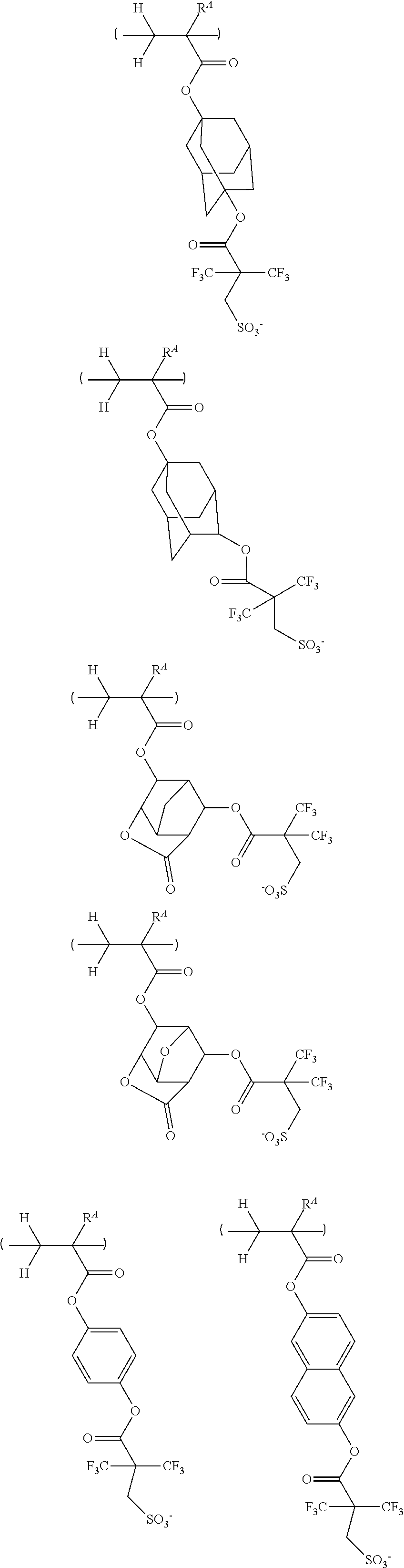

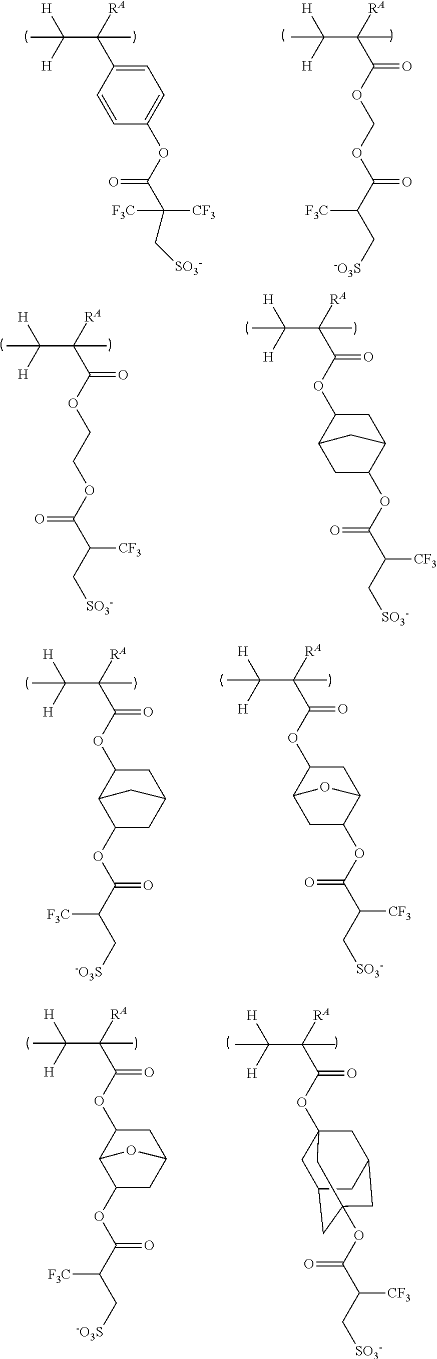

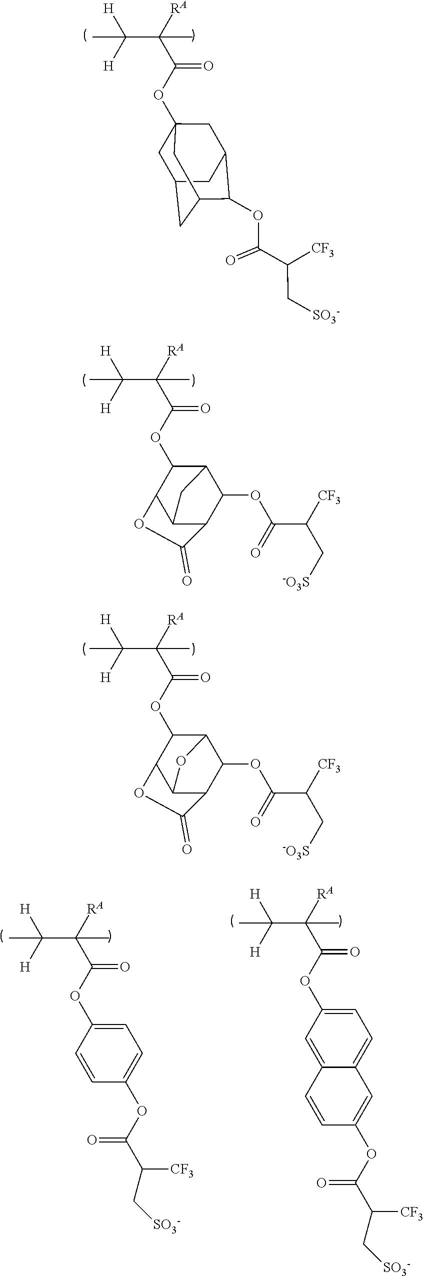

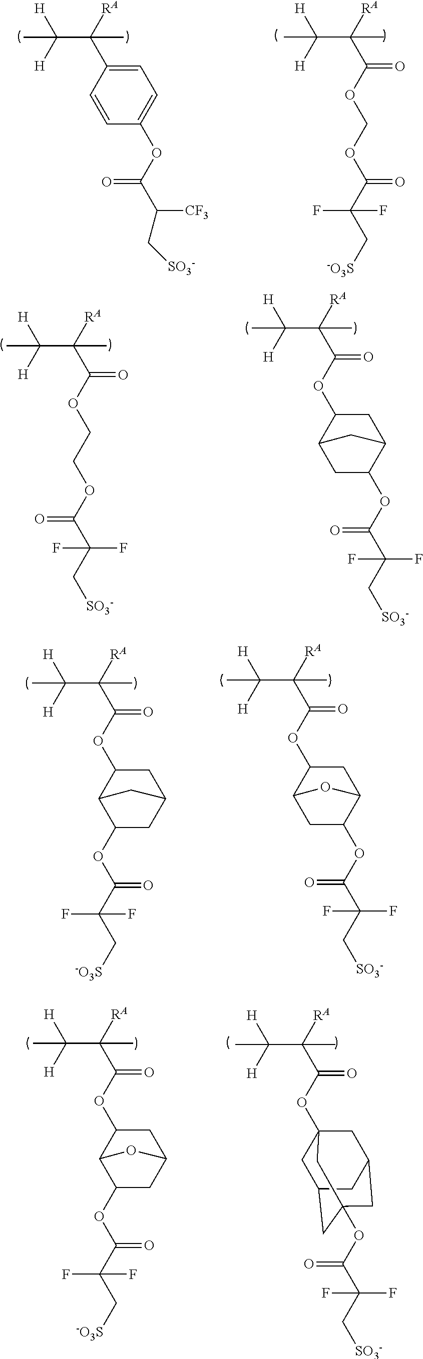

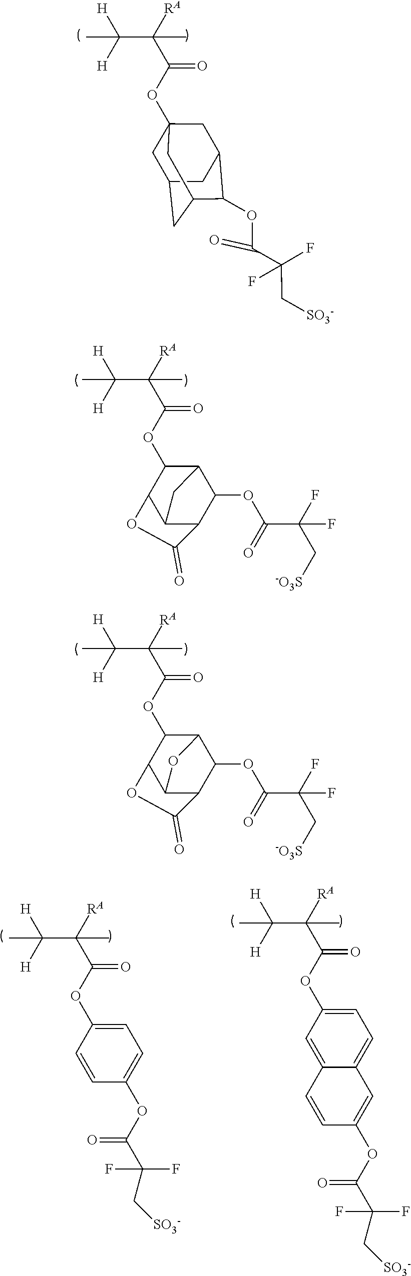

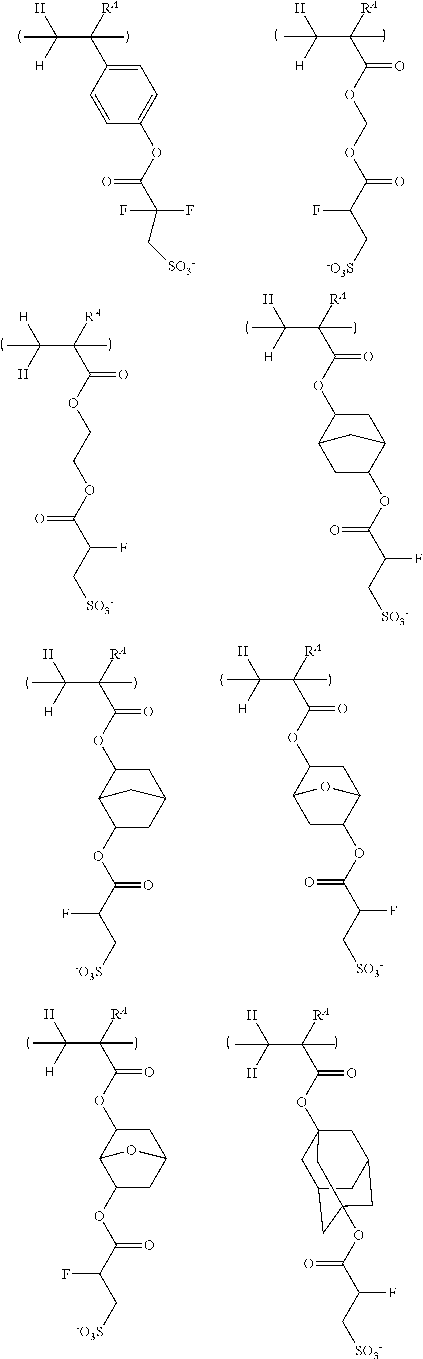

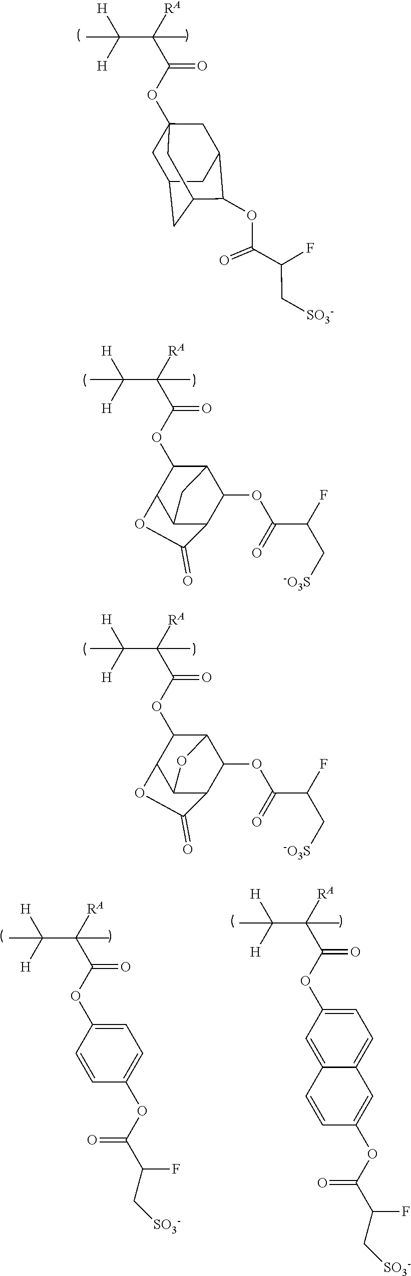

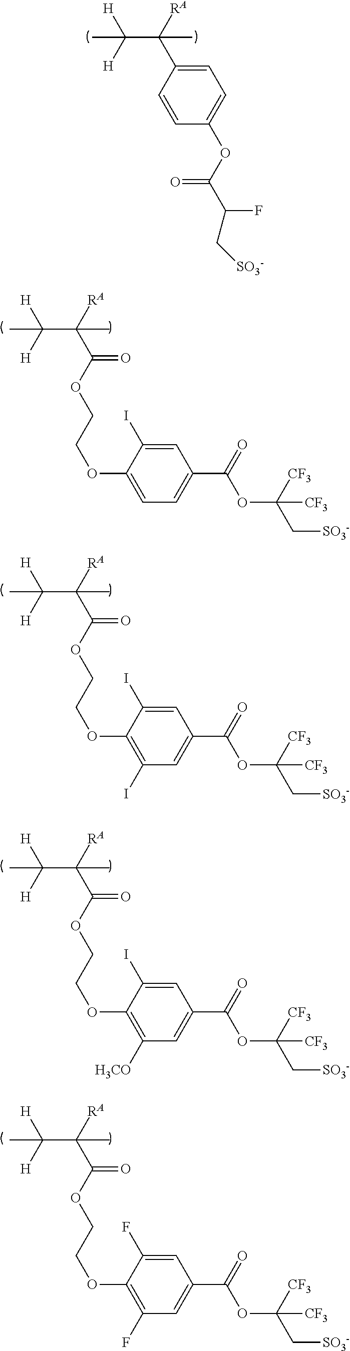

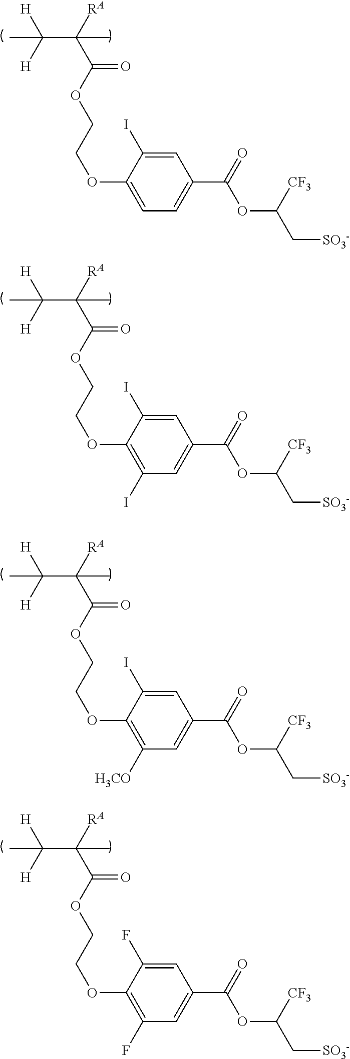

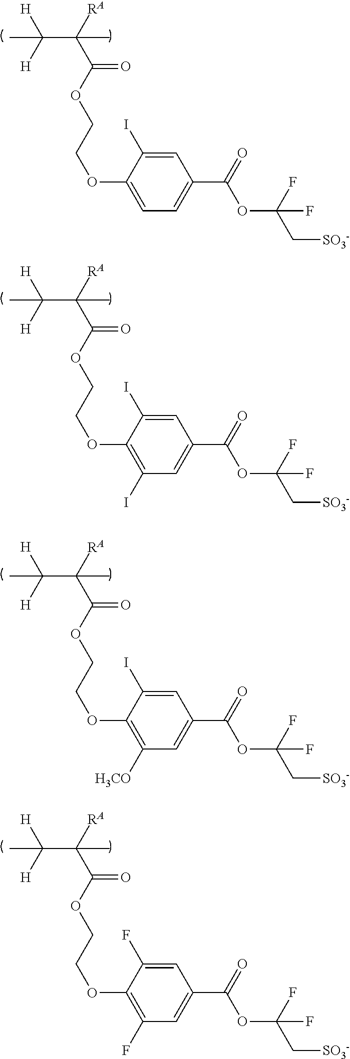

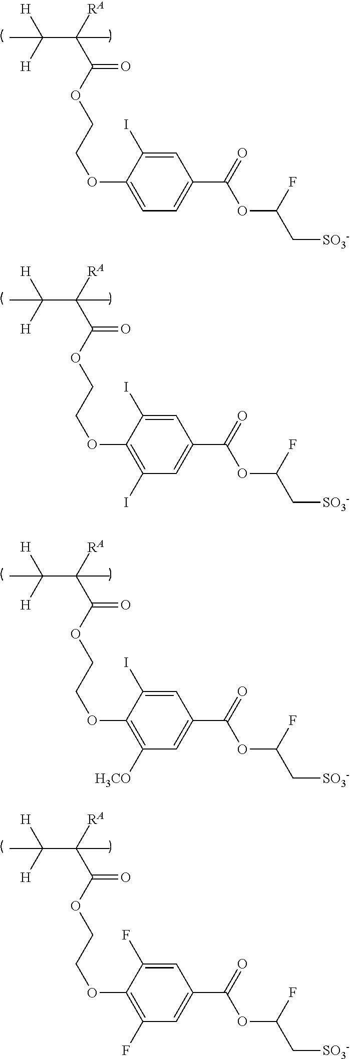

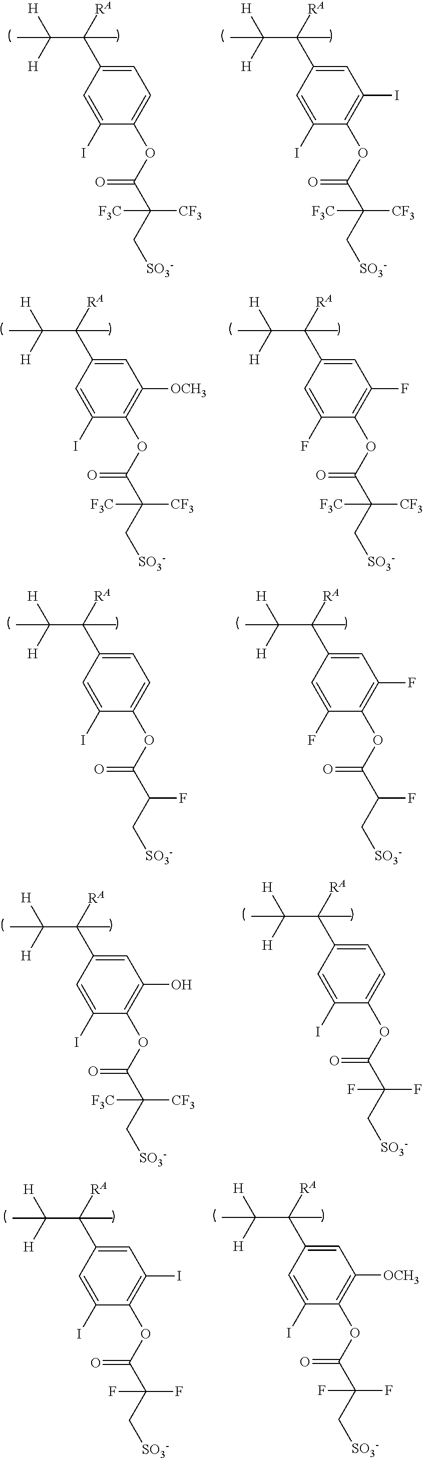

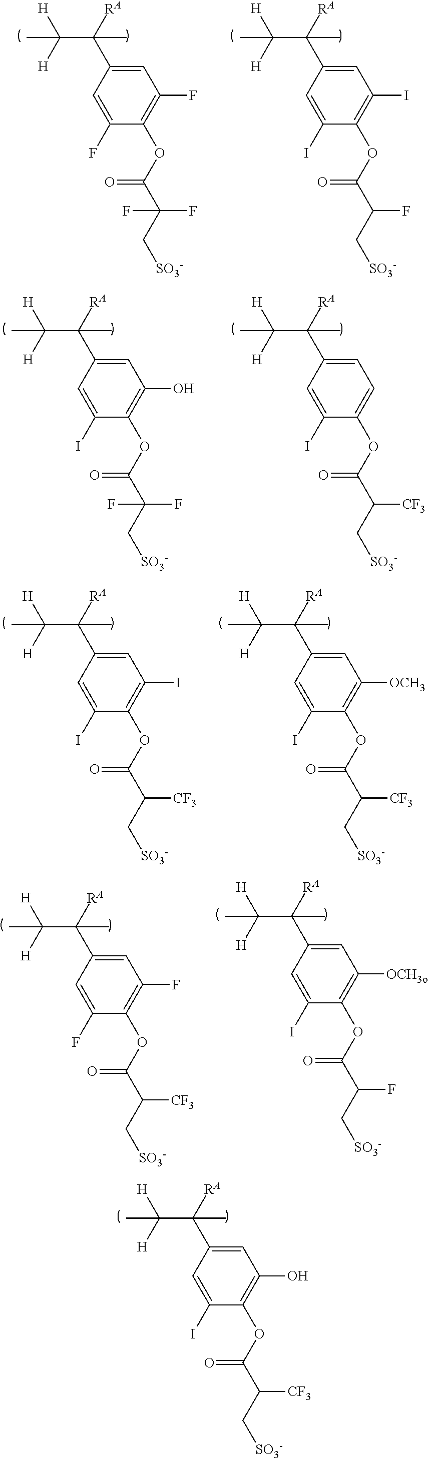

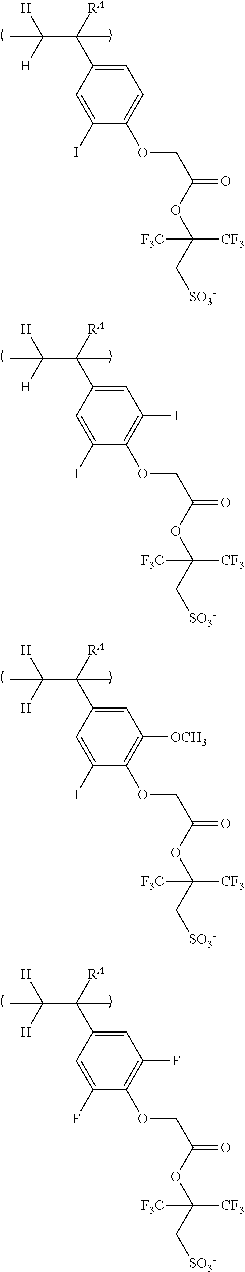

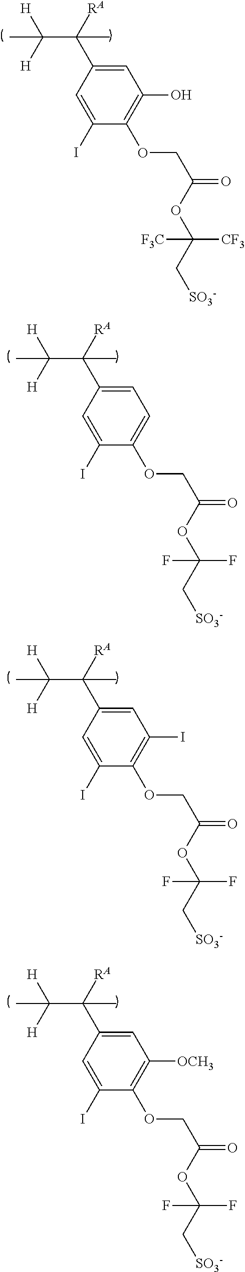

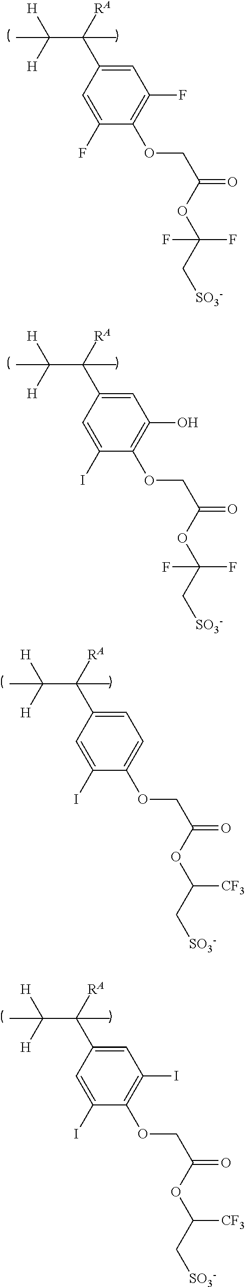

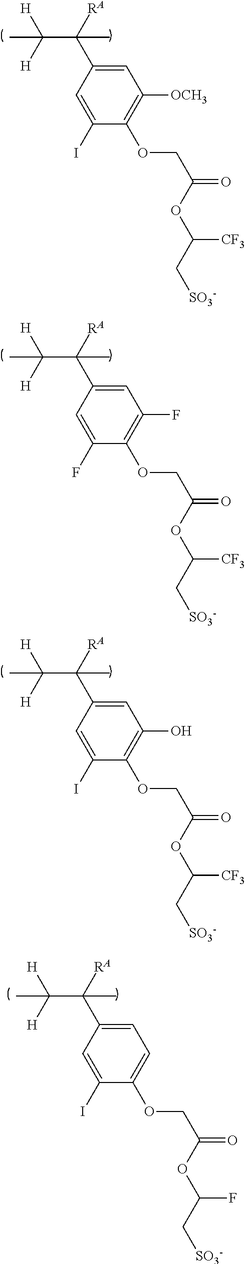

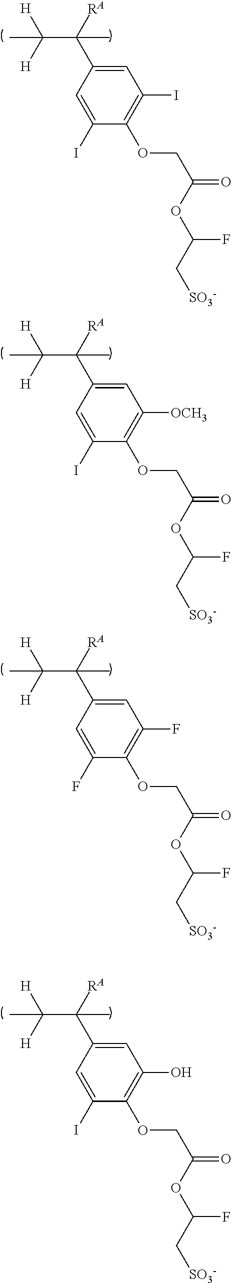

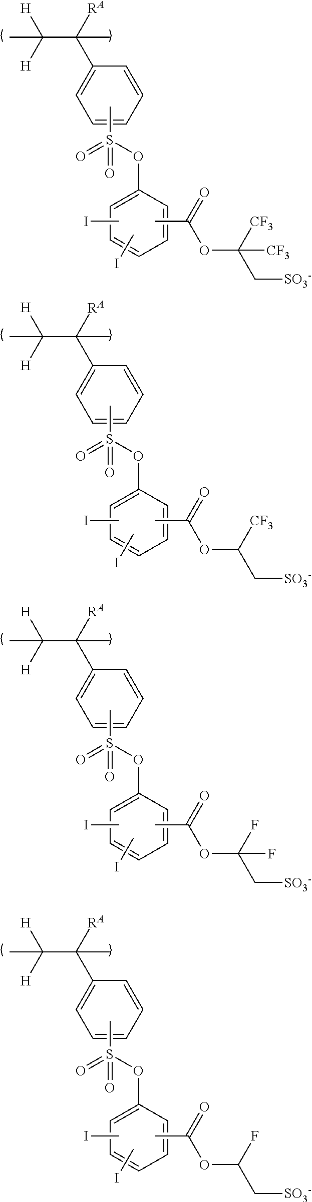

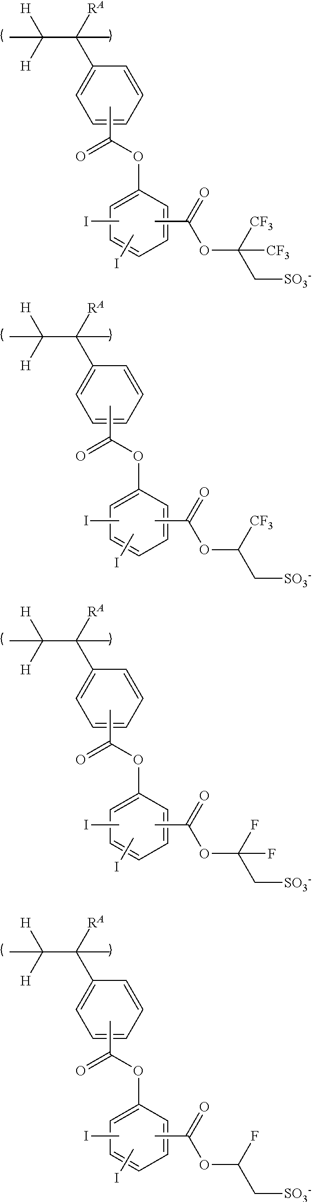

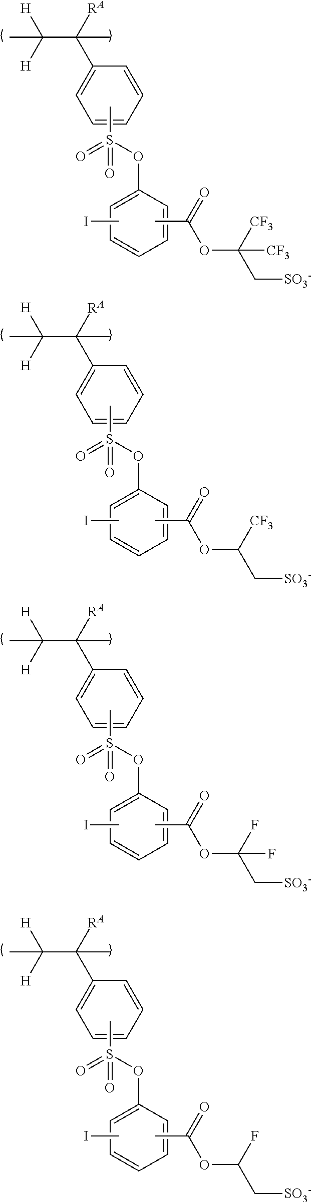

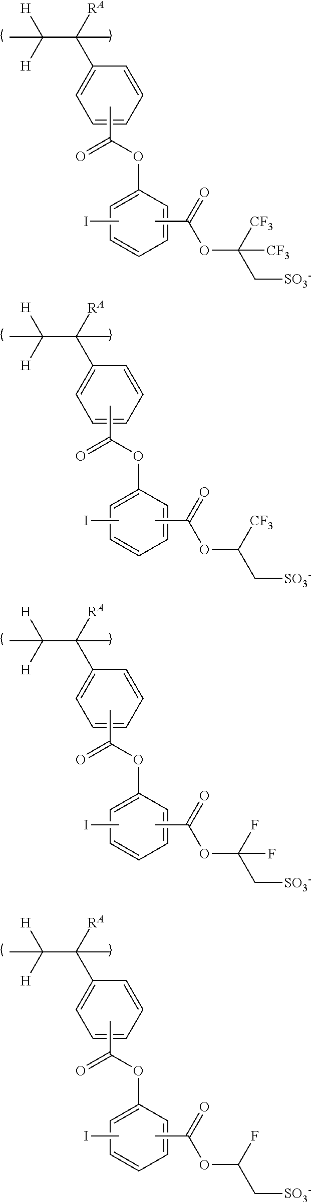

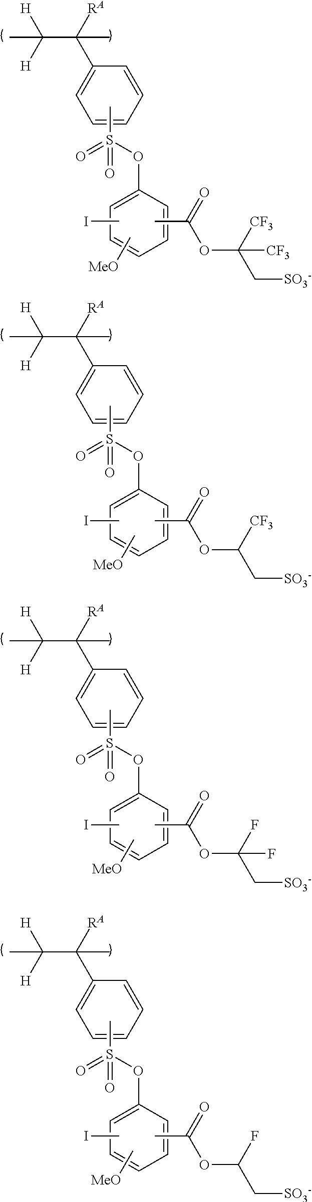

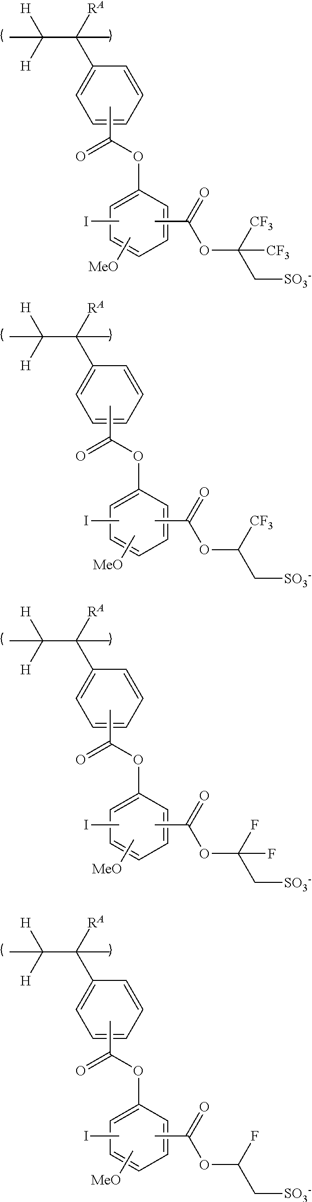

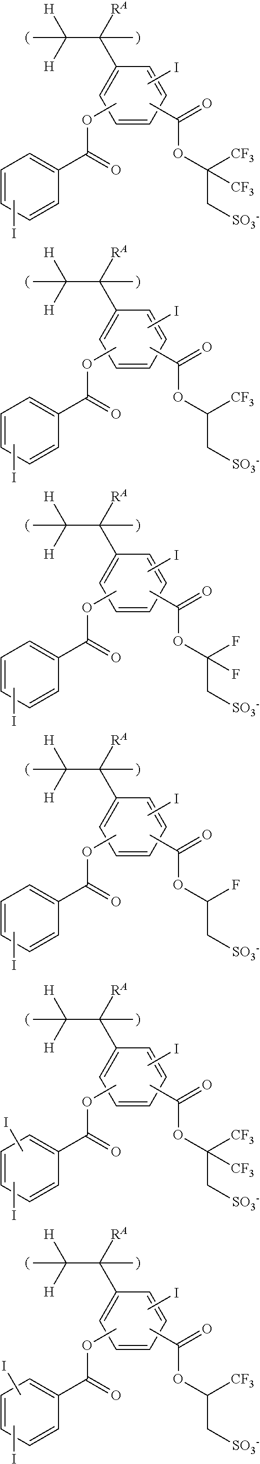

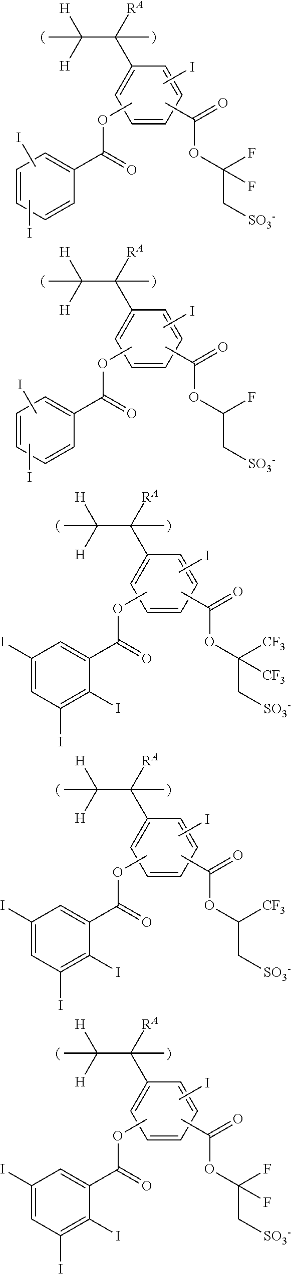

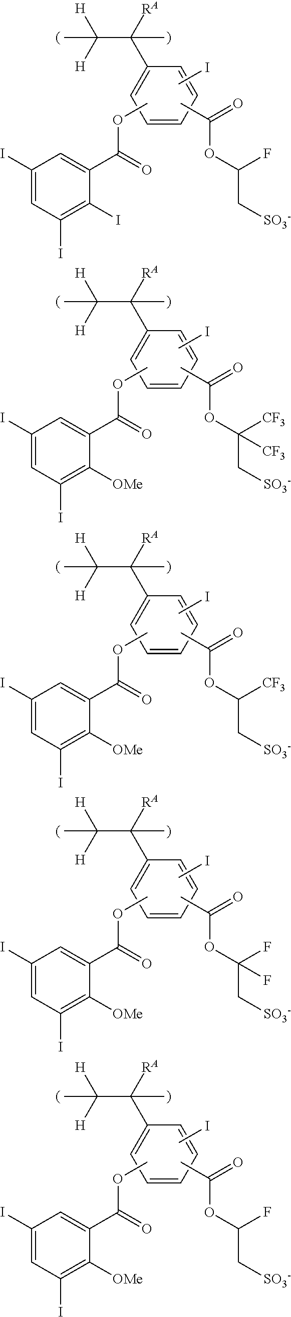

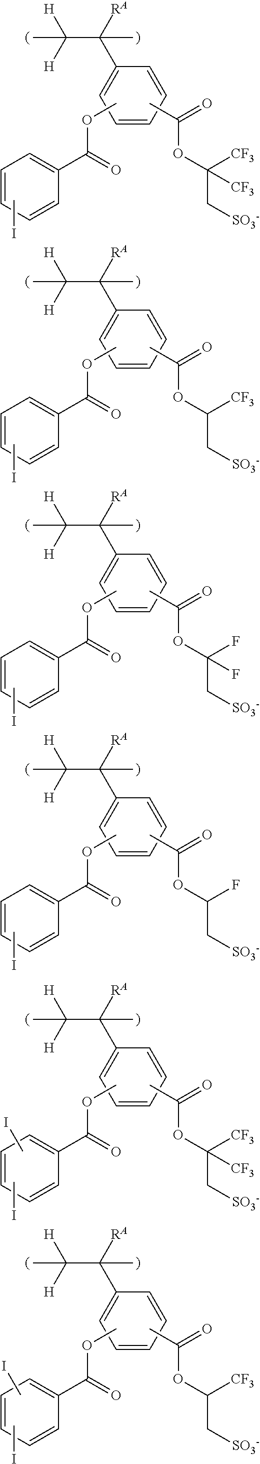

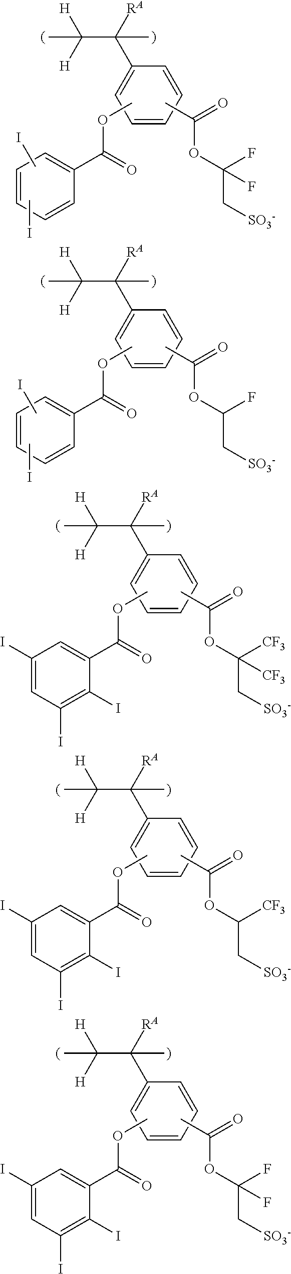

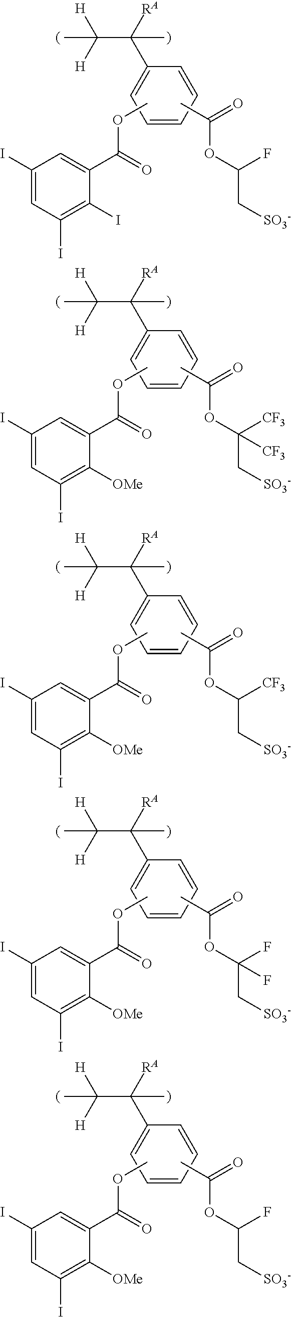

Examples of the anion in repeat unit (c4) are shown below, but not limited thereto. R^{A} is as defined above.

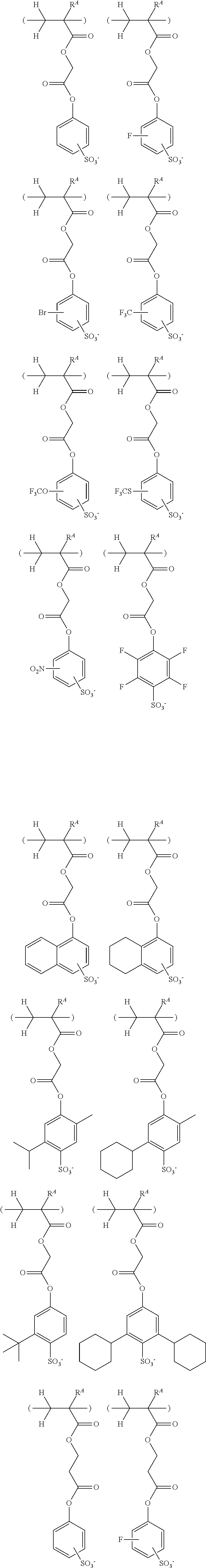

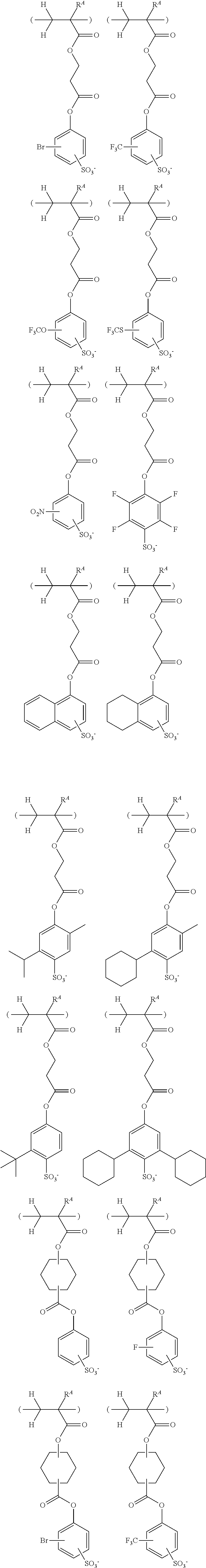

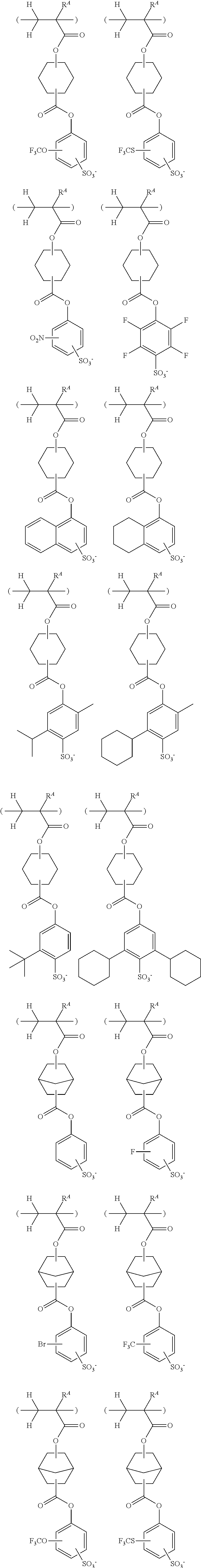

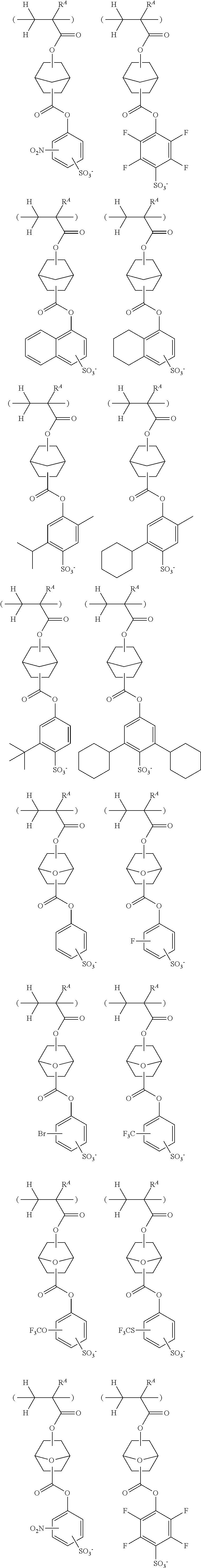

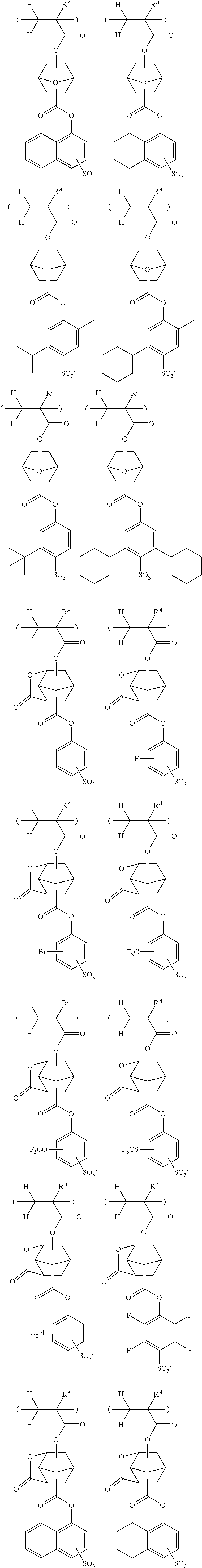

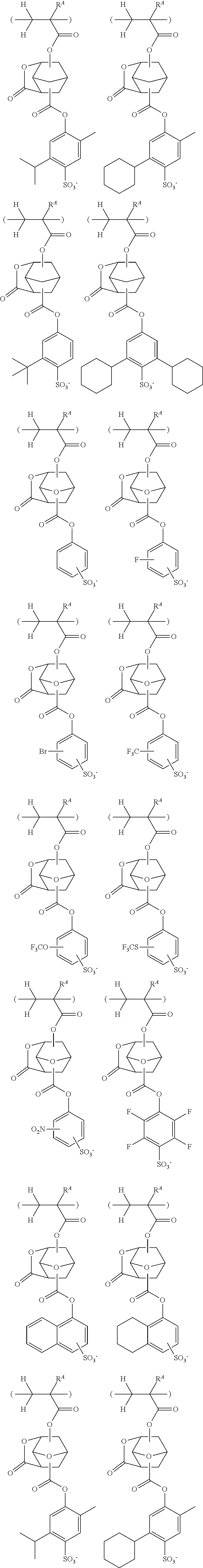

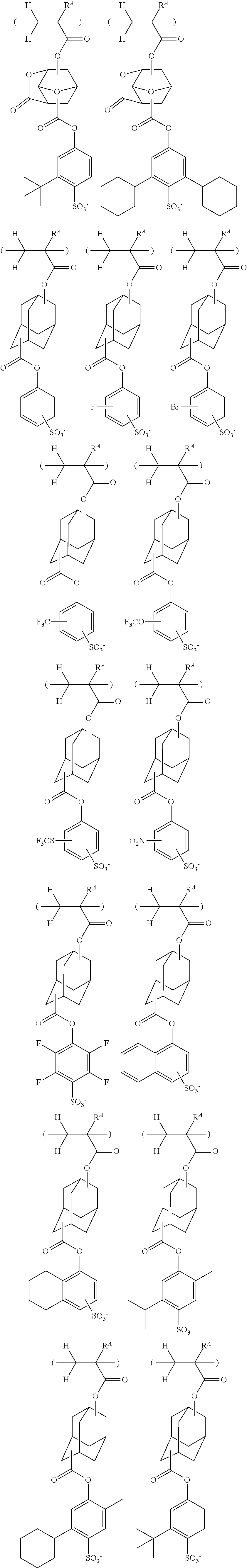

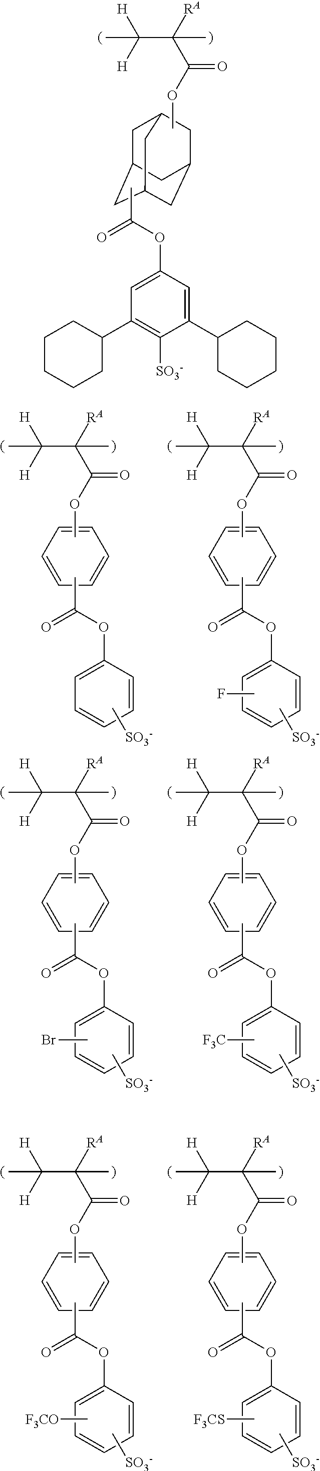

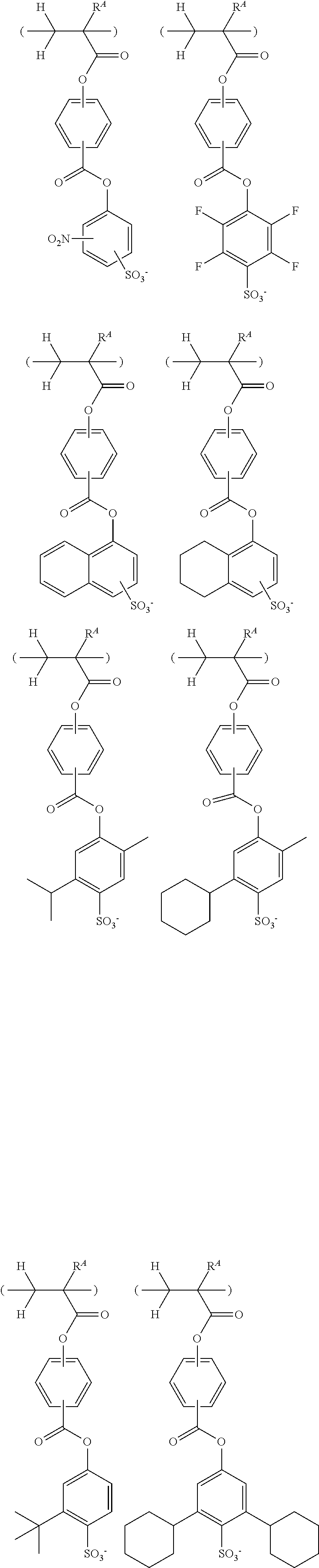

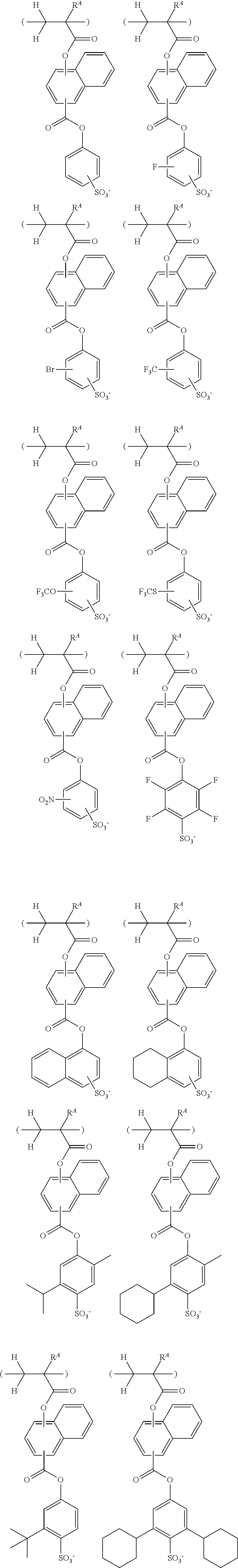

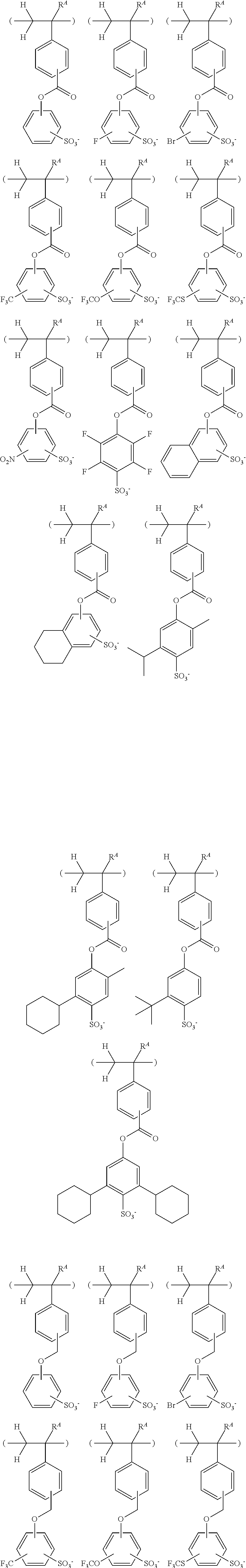

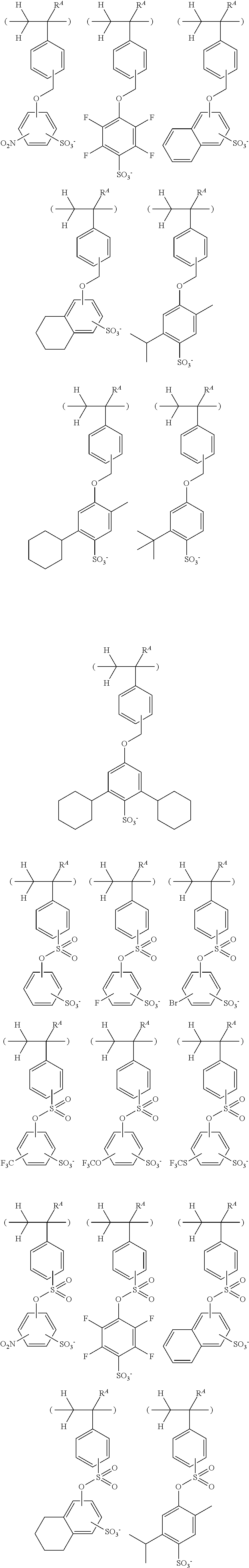

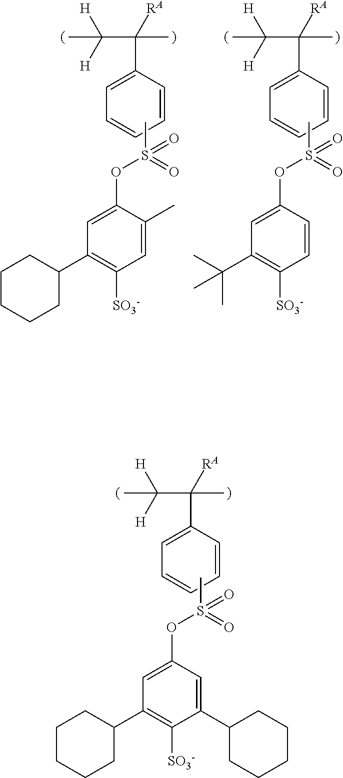

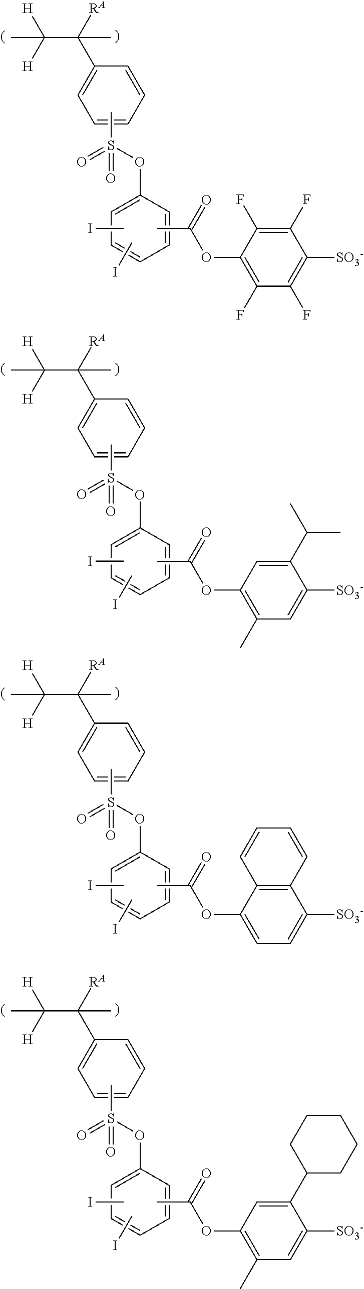

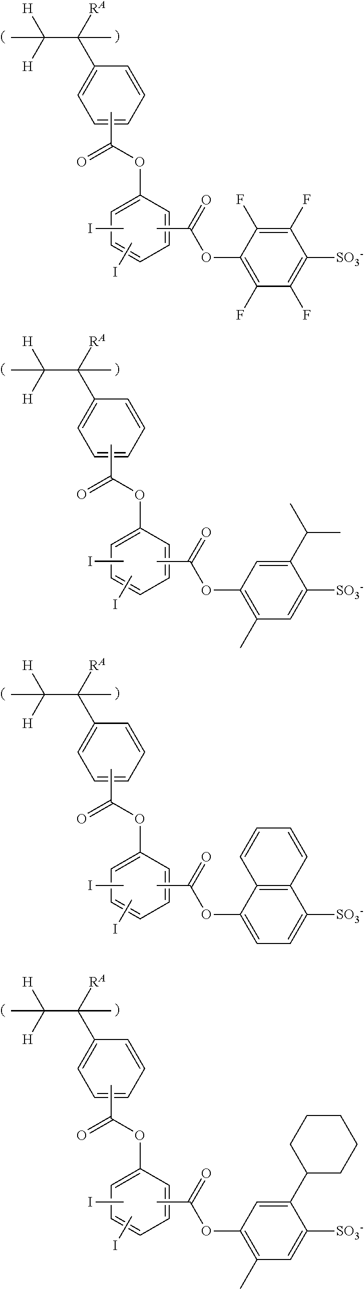

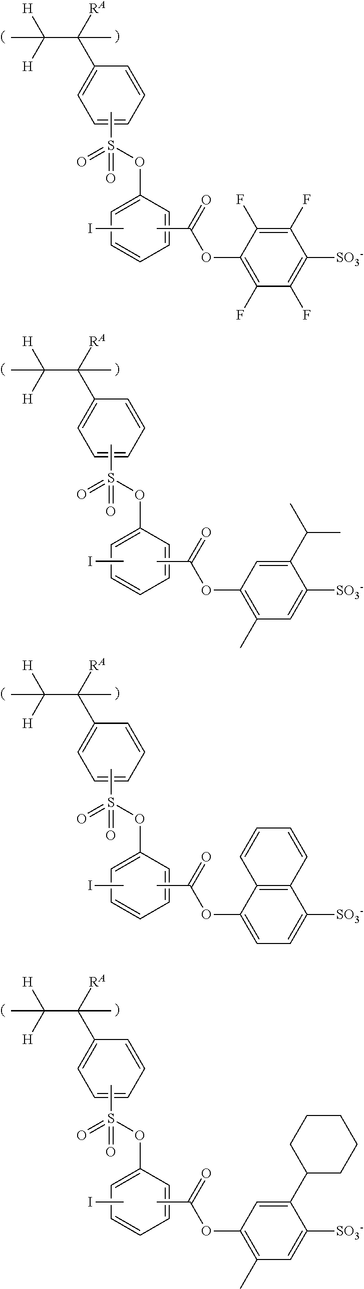

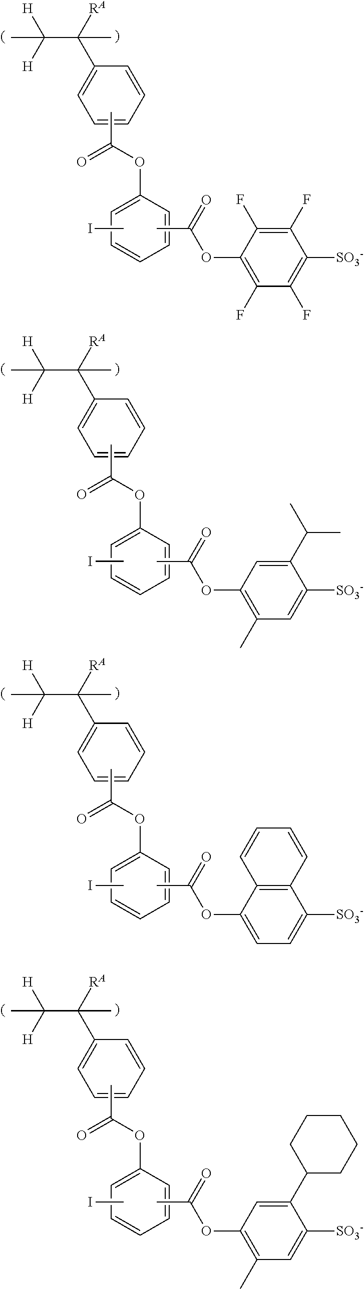

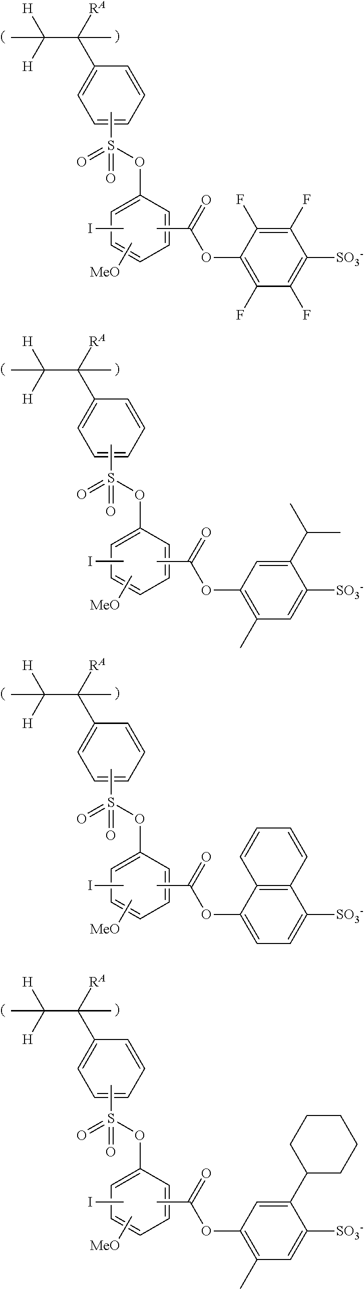

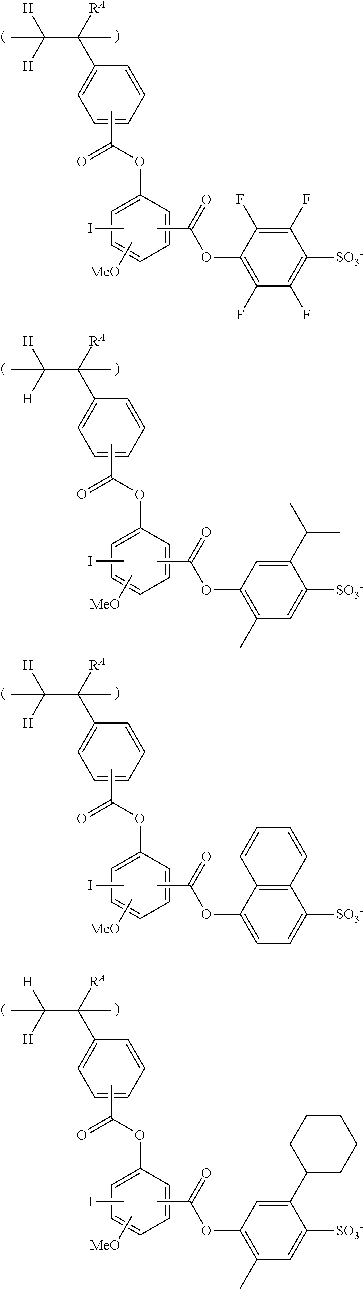

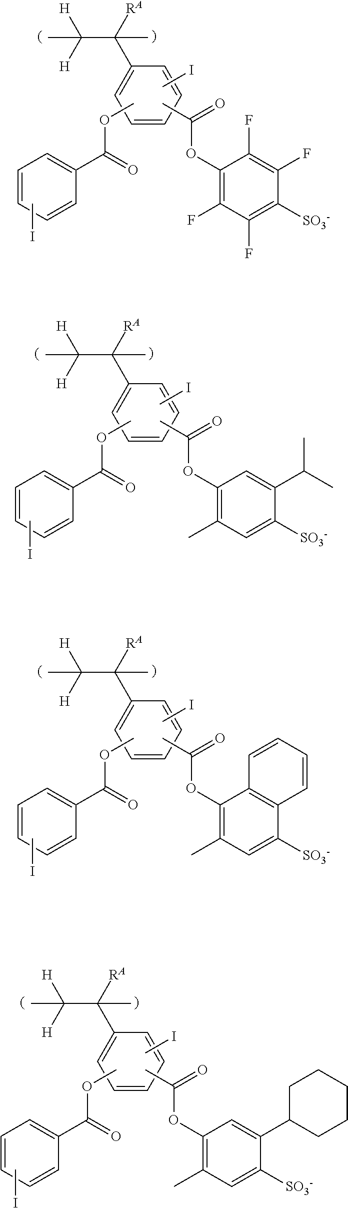

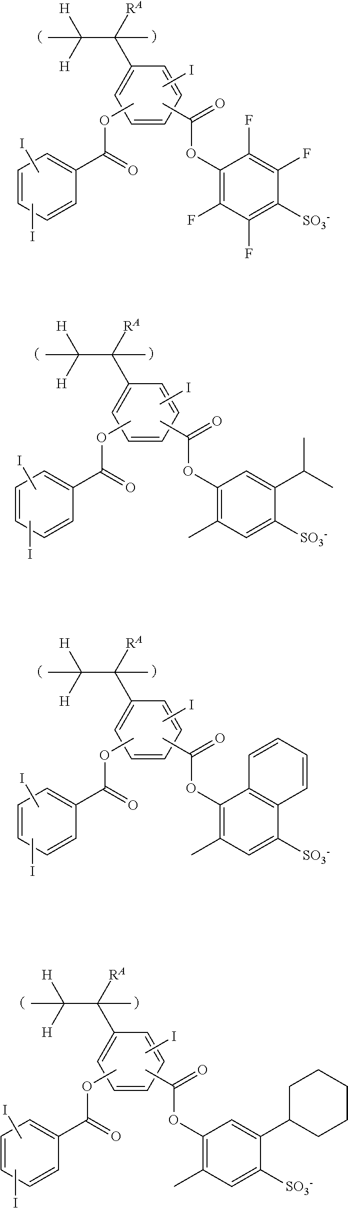

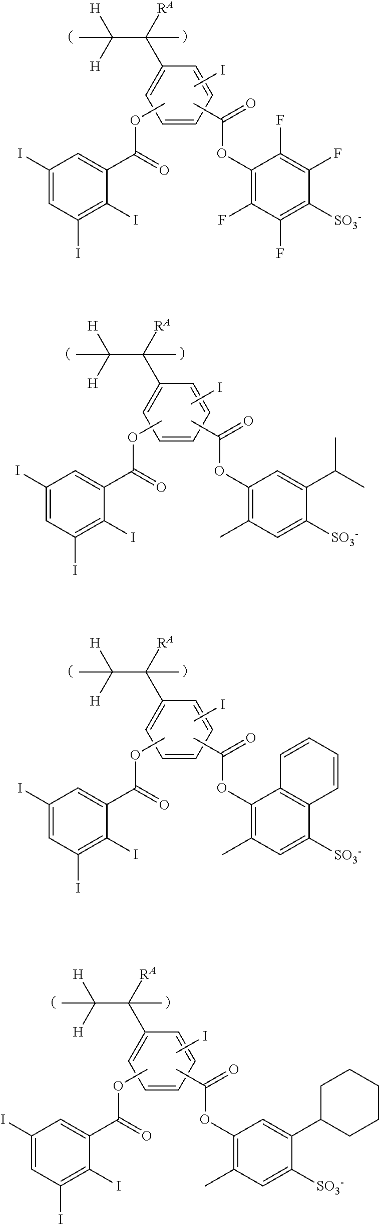

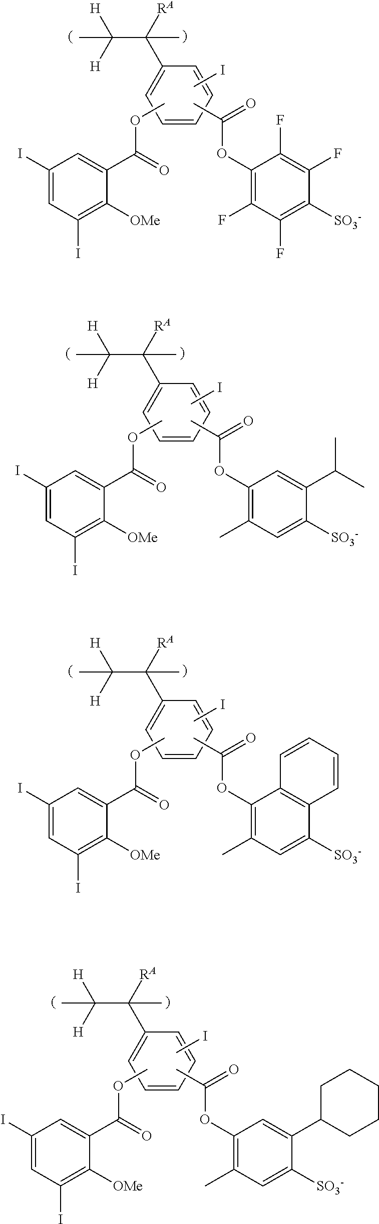

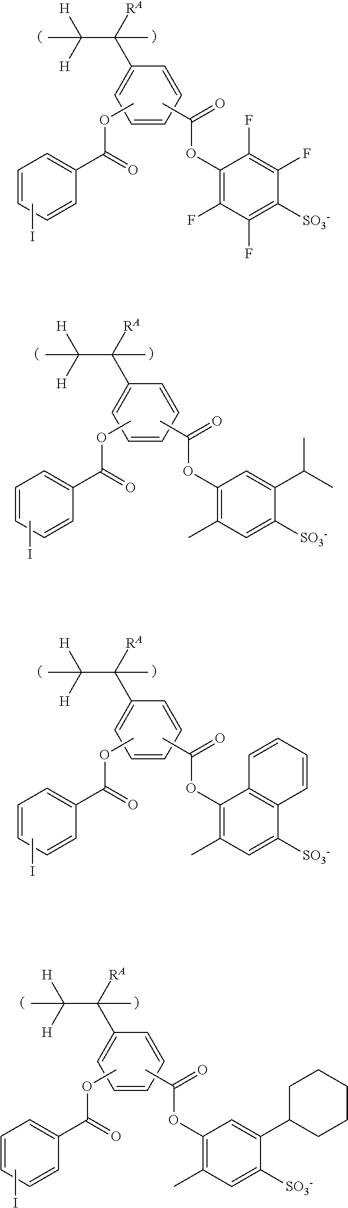

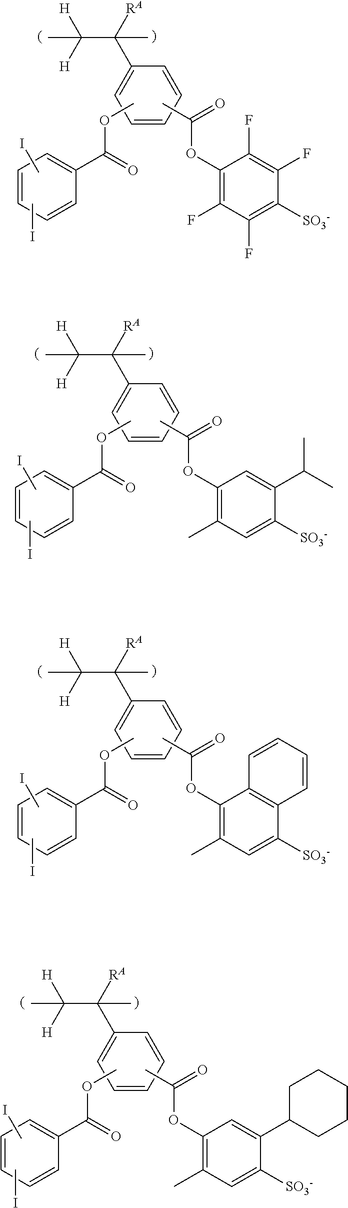

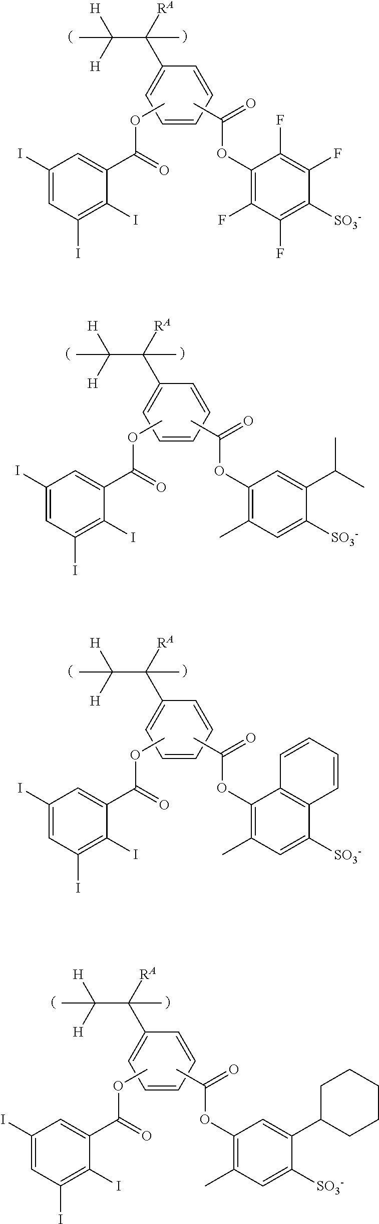

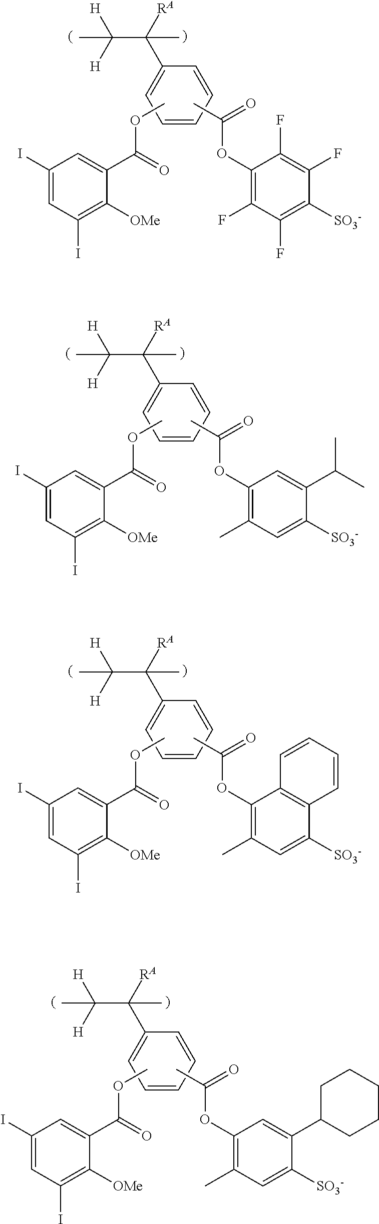

Examples of the anion in repeat unit (c5) are shown below, but not limited thereto. R^{A} is as defined above.

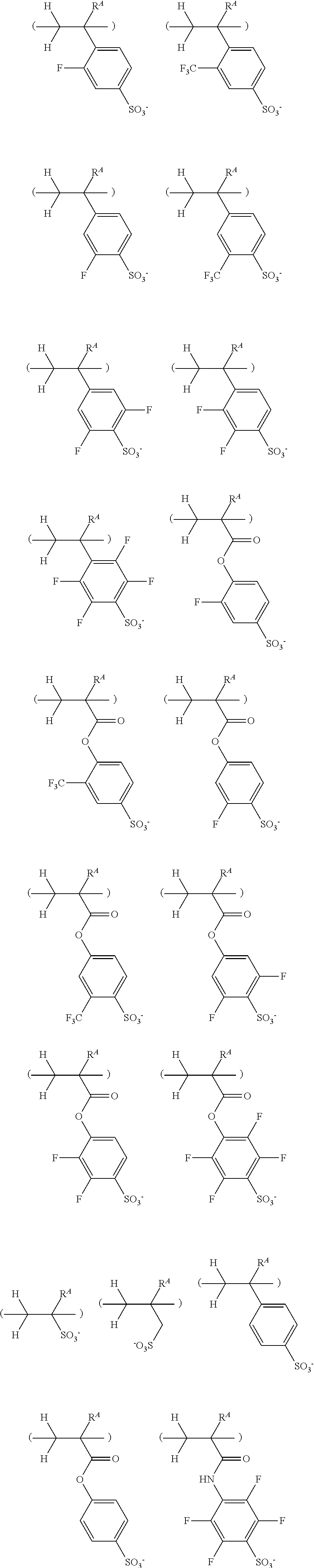

In formulae (c2) to (c5), A⁺ is an onium cation. Suitable onium cations include sulfonium, iodonium and ammonium cations, with the sulfonium and iodonium cations being preferred. Non-limiting examples of the sulfonium cation include those exemplified above as the cation in the sulfonium salt having formula (1), those described in JP-A 2024-003744, paragraphs [0102]-[0125], WO 2024/128017, paragraphs [0044]-[0049], and JP 7491173, paragraphs [0035]-[0046].

A sulfonium cation having the formula (sulfo-1) is also preferred.

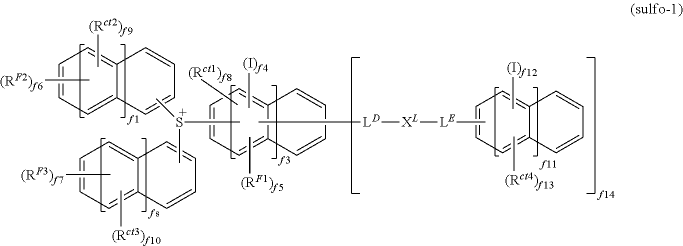

In formula (sulfo-1), f1 is 0 or 1. The relevant structure is a benzene ring when f1-0 and a naphthalene ring when f1=1. From the aspect of solvent solubility, the benzene ring corresponding to f1=0 is preferred. The subscript f2 is 0 or 1. The relevant structure is a benzene ring when f2-0 and a naphthalene ring when f2=1. From the aspect of solvent solubility, the benzene ring corresponding to f2-0 is preferred. The subscript f3 is 0 or 1. The relevant structure is a benzene ring when f3-0 and a naphthalene ring when f3=1. From the aspect of solvent solubility, the benzene ring corresponding to f3-0 is preferred.

In formula (sulfo-1), f4 is 0, 1, 2, 3 or 4. Since a cation structure containing more iodine atoms is more absorptive to EUV, but loses solvent solubility so that it may precipitate in a resist composition, it is preferred that f4 be 0, 1, 2 or 3, more preferably 0, 1 or 2.

In formula (sulfo-1), f5 is 0, 1, 2, 3 or 4. It is preferred from the aspect of reactant availability that f5 be 0, 1, 2 or 3, more preferably 0, 1 or 2. The subscript f6 is 0, 1, 2, 3, 4, 5 or 6. It is preferred from the aspect of reactant availability that f6 be 0, 1, 2 or 3, more preferably 0, 1 or 2. The subscript f7 is 0, 1, 2, 3, 4, 5 or 6. It is preferred from the aspect of reactant availability that f7 be 0, 1, 2 or 3, more preferably 0, 1 or 2.

In formula (sulfo-1), f8 is 0, 1 or 2. It is preferred from the aspect of reactant availability that f8 be 0 or 1. The subscript f9 is 0, 1 or 2. It is preferred from the aspect of reactant availability that f9 be 0 or 1. The subscript f10 is 0, 1 or 2. It is preferred from the aspect of reactant availability that f10 be 0 or 1.

In formula (sulfo-1), f11 is 0 or 1. The relevant structure is a benzene ring when f11-0 and a naphthalene ring when f11-1. From the aspect of solvent solubility, the benzene ring corresponding to f11=0 is preferred.

In formula (sulfo-1), f12 is 0, 1, 2, 3 or 4. Since a cation structure containing more iodine atoms is more absorptive to EUV, but loses solvent solubility so that it may precipitate in a resist composition, it is preferred that f12 be 0, 1, 2 or 3, more preferably 0, 1 or 2.

In formula (sulfo-1), f13 is 0, 1 or 2. It is preferred from the aspect of reactant availability that f13 be 0 or 1. The subscript f14 is 0, 1 or 2. It is preferred from the aspect of synthesis that f14 be 0 or 1.

The subscripts f1 to f13 are in the range: 0≤f6+f9≤4 when f1=0, and 0≤f6+f9≤6 when f1=1; 0≤f7+f10≤4 when f2=0, and 0≤f7+f10≤6 when f2=1; 1≤f4+f5+f8+f14≤4 when f3=0, and 1≤f4+f5+f8+f14≤6 when f3=1; 0≤f12+f13≤4 when f11=0, and 0≤f12+f13≤6 when f11=1; and f4+f12≥1.

In formula (sulfo-1), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl, trifluoromethoxy, and trifluorothiomethoxy are preferred. A plurality of R^{F1} may be identical or different when f5 is 2, 3 or 4, a plurality of R^{F2} may be identical or different when f6 is 2, 3, 4, 5 or 6, and a plurality of R^{F3} may be identical or different when f7 is 2, 3, 4, 5 or 6.

In formula (sulfo-1), R^{ct1} to R^{ct4} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof are as exemplified above for the hydrocarbyl group R¹ to R³ in formula (1). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When f8=2, two R^{ct1} may be identical or different and two R^{q11} may bond together to form a ring with the carbon atoms to which they are attached. When f9=2, two R^{ct2} may be identical or different and two R^{ct2} may bond together to form a ring with the carbon atoms to which they are attached. When f10=2, two R^{ct3} may be identical or different and two R^{ct3} may bond together to form a ring with the carbon atoms to which they are attached. When f13=2, two R^{ct4} may be identical or different and two R^{ct4} may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some —H₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having formula (sulfo-1) may bond together to form a ring with S⁺. Exemplary structures of the ring are shown below.

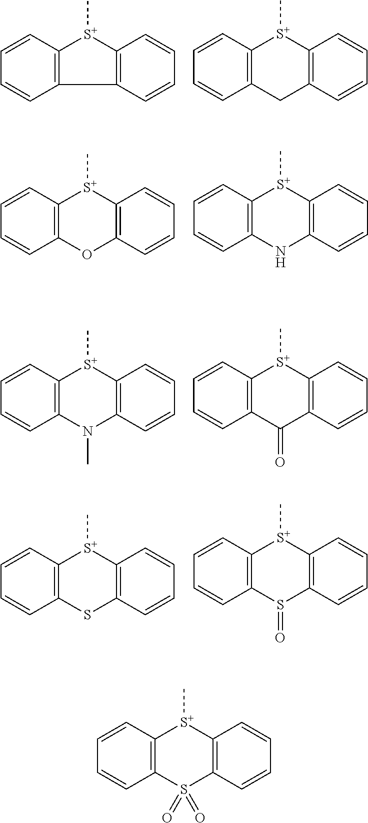

In formula (sulfo-1), L^{D} and L^{E} are each independently a single bond, ether bond, ester bond, sulfonate ester bond, amide bond, sulfonamide bond, carbonate bond or carbamate bond. L^{D} is preferably a single bond, ether bond, ester bond or sulfonate ester bond, more preferably an ester bond or sulfonate ester bond. L^{E} is preferably a single bond, ether bond or ester bond, more preferably a single bond.

In formula (sulfo-1), X^{L} is a single bond or a C₁-C₄₀ hydrocarbylene group which may contain a heteroatom. The hydrocarbylene group may be straight, branched or cyclic. Examples thereof include alkanediyl, cyclic saturated hydrocarbylene, and arylene groups. Suitable heteroatoms include oxygen, nitrogen and sulfur.

Examples of the optionally heteroatom-containing C₁-C₄₀ hydrocarbylene group X^{L} are shown below, but not limited thereto. Herein, * designates a point of attachment to L^{D} or L^{E}.

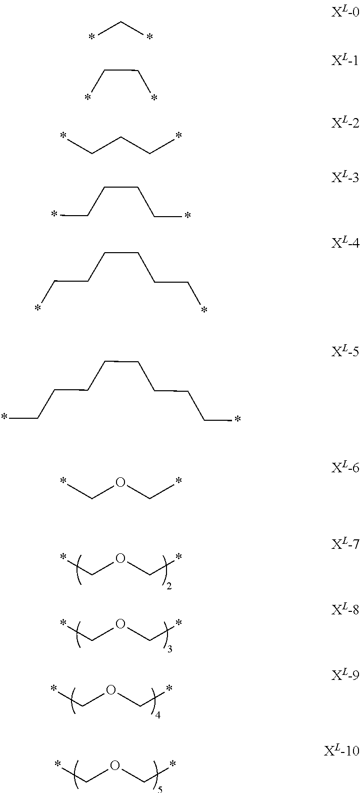

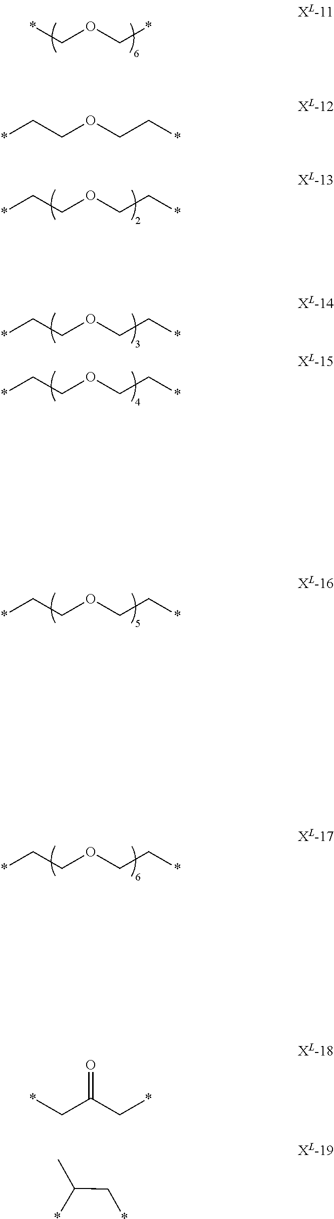

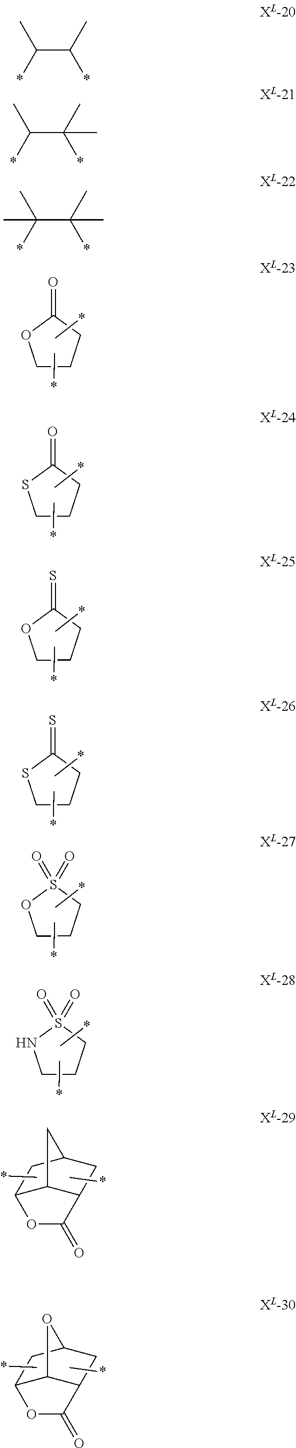

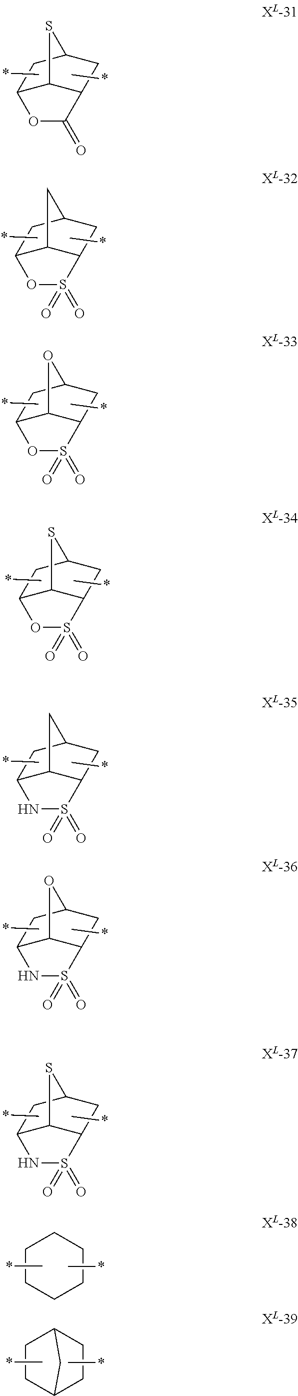

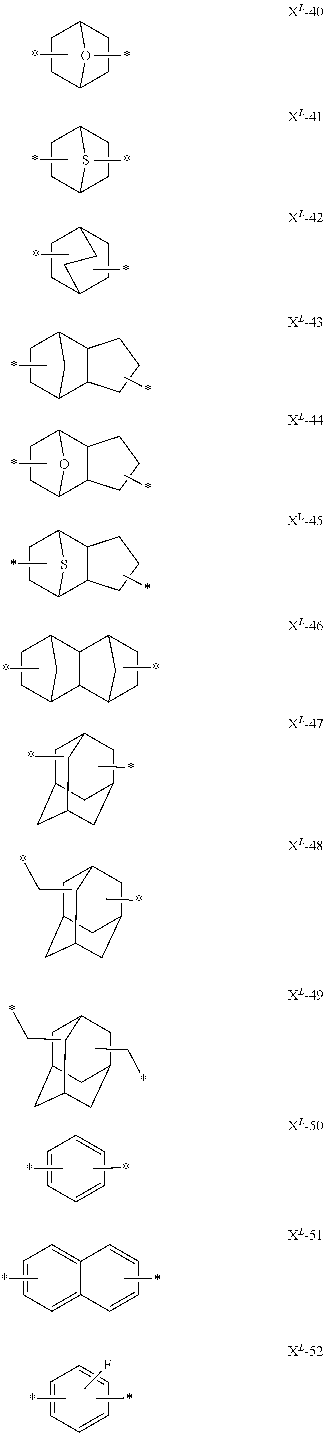

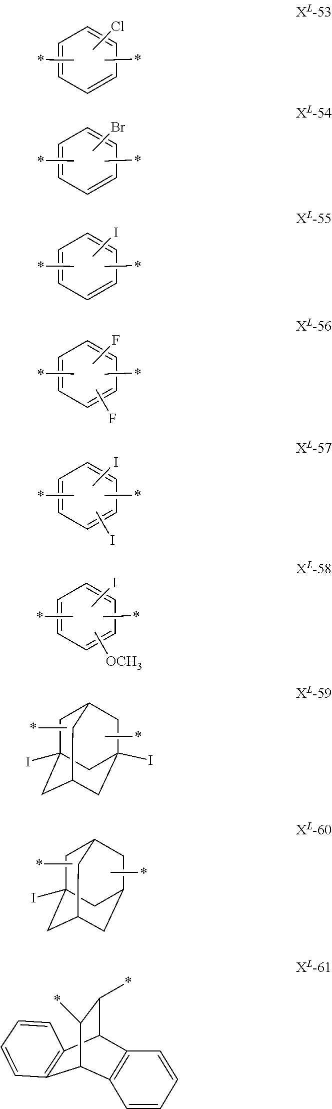

Of these, X^{L}-0 to X^{L}-22, X^{L}-29 to X^{L}-34, and X^{L}-47 to X^{L}-61 are preferred as X¹².

Of the sulfonium cations having formula (sulfo-1), those having the formula (sulfo-1-1) are preferred:

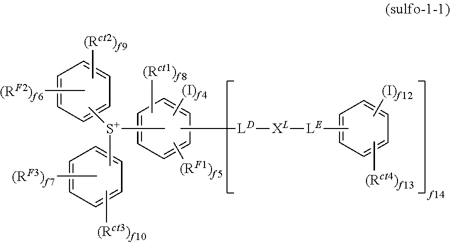

wherein f4 to f10, f12 to f14, R^{F1} to R^{F3}, R^{ct1} to R^{ct4}, L^{D}, L^{E}, and X^{L} are as defined above.

Of the sulfonium cations having formula (sulfo-1-1), those having the formula (sulfo-1-2) are more preferred:

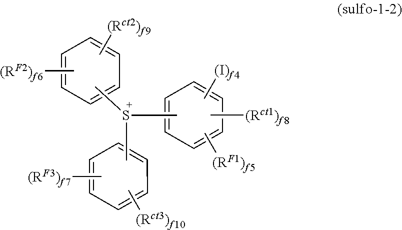

wherein f4 to f10, R^{F1} to R^{F3}, and R^{ct1} to R^{ct3} are as defined above.

Examples of the sulfonium cation having formula (sulfo-1) are shown below, but not limited thereto.

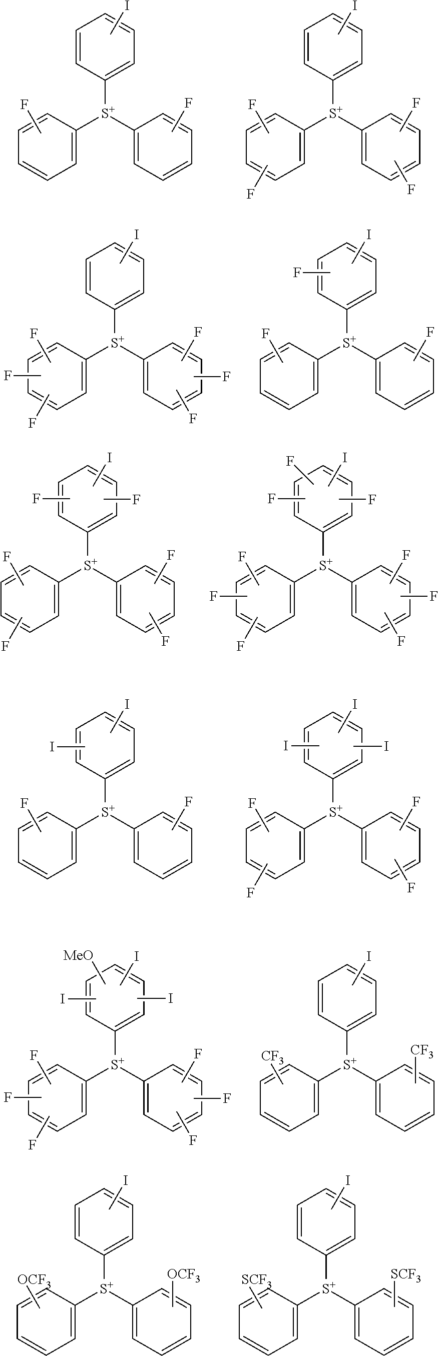

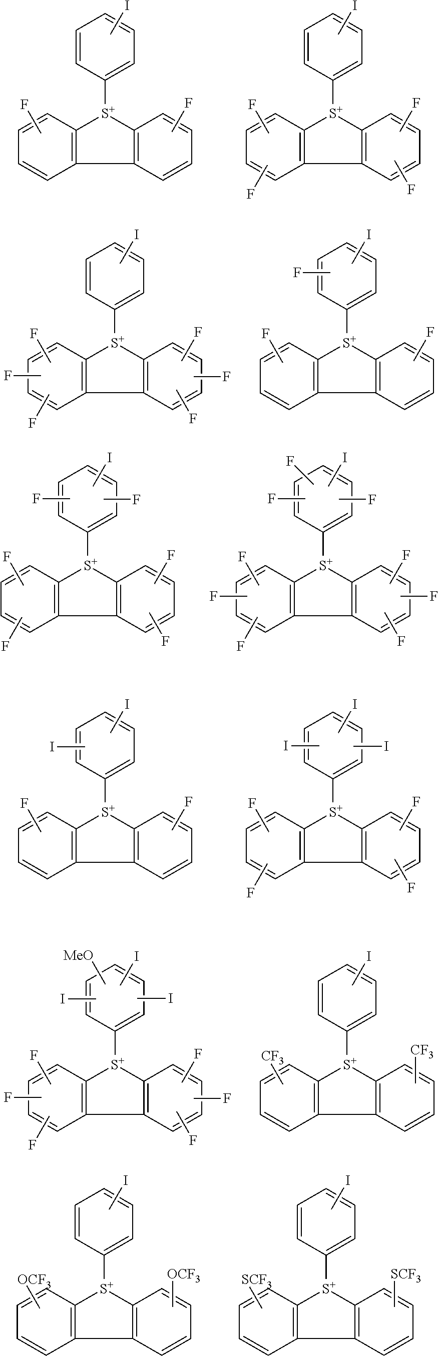

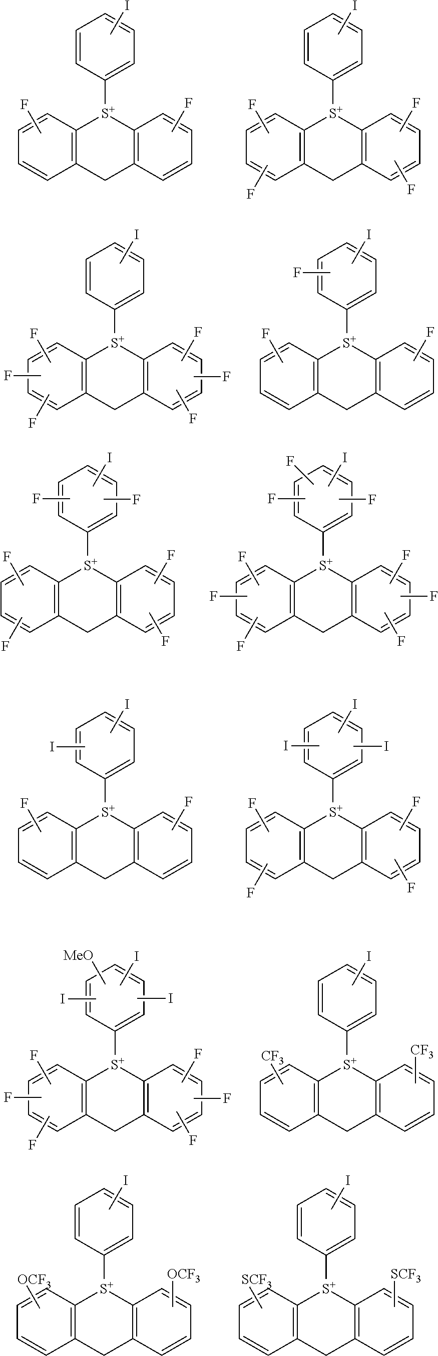

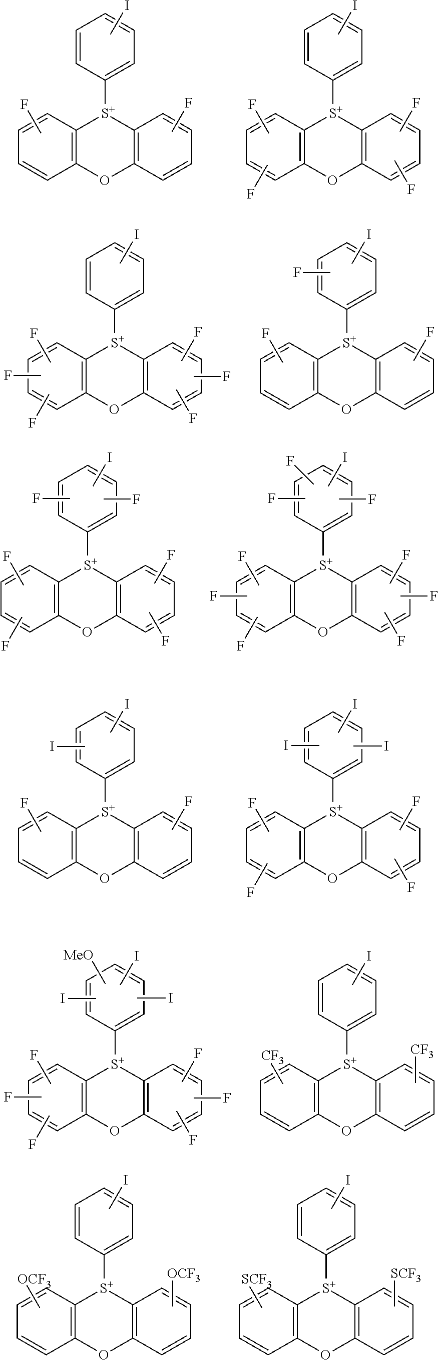

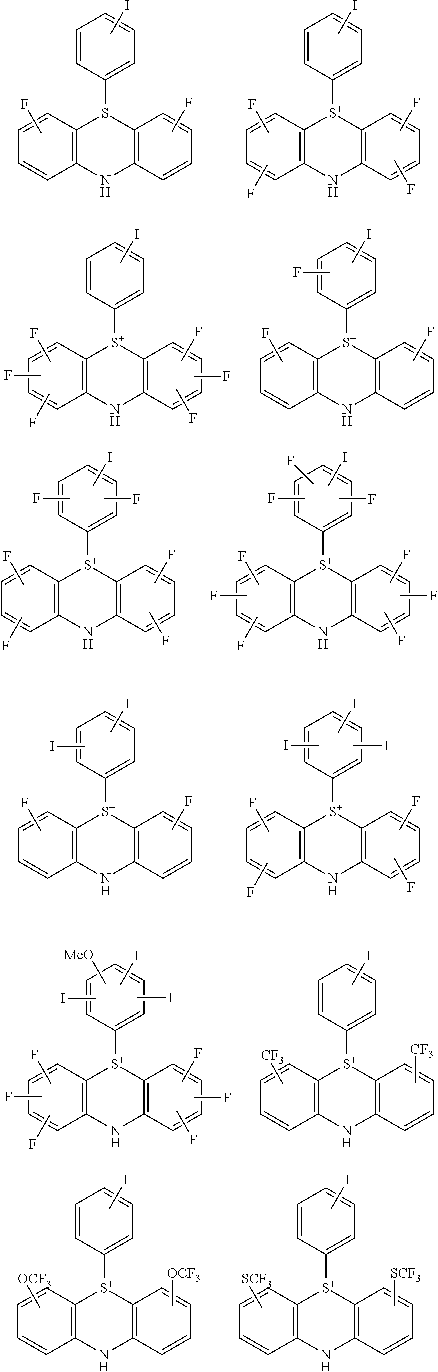

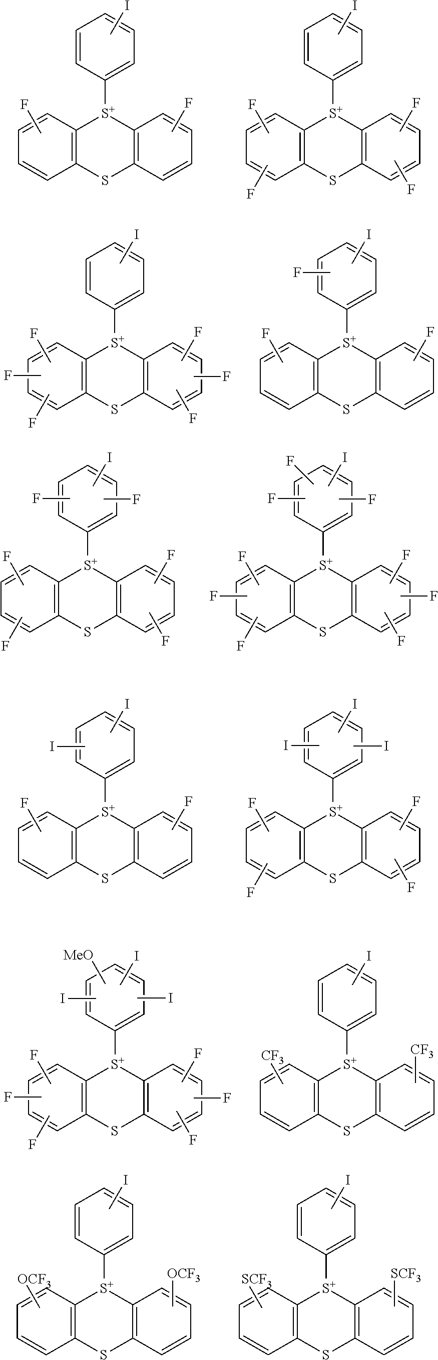

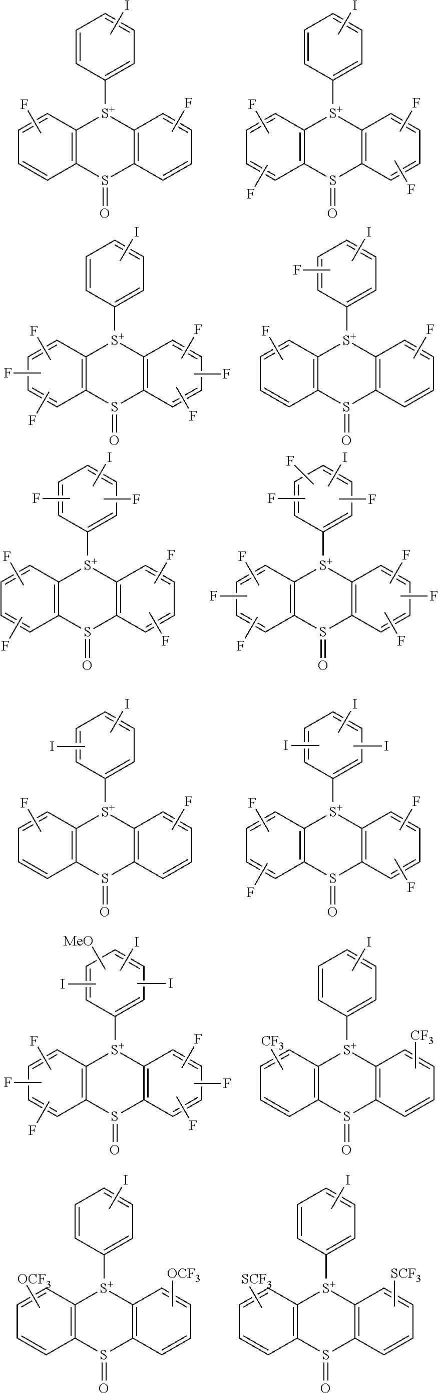

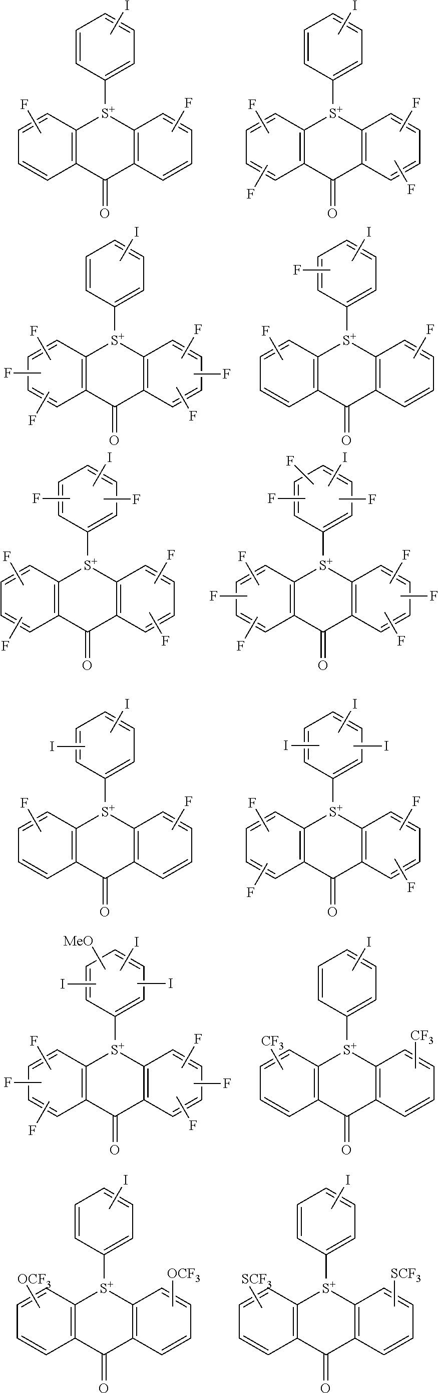

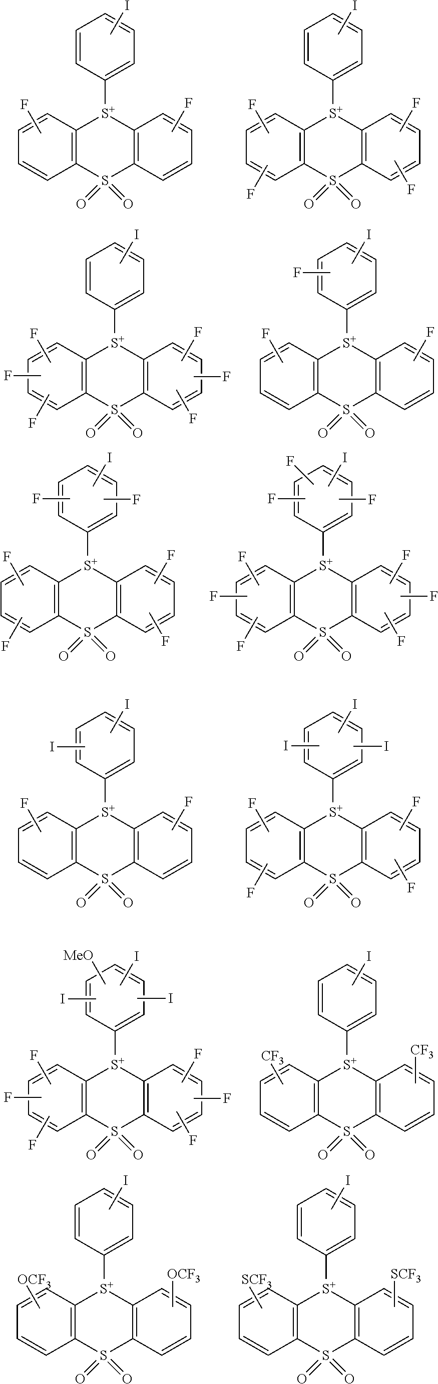

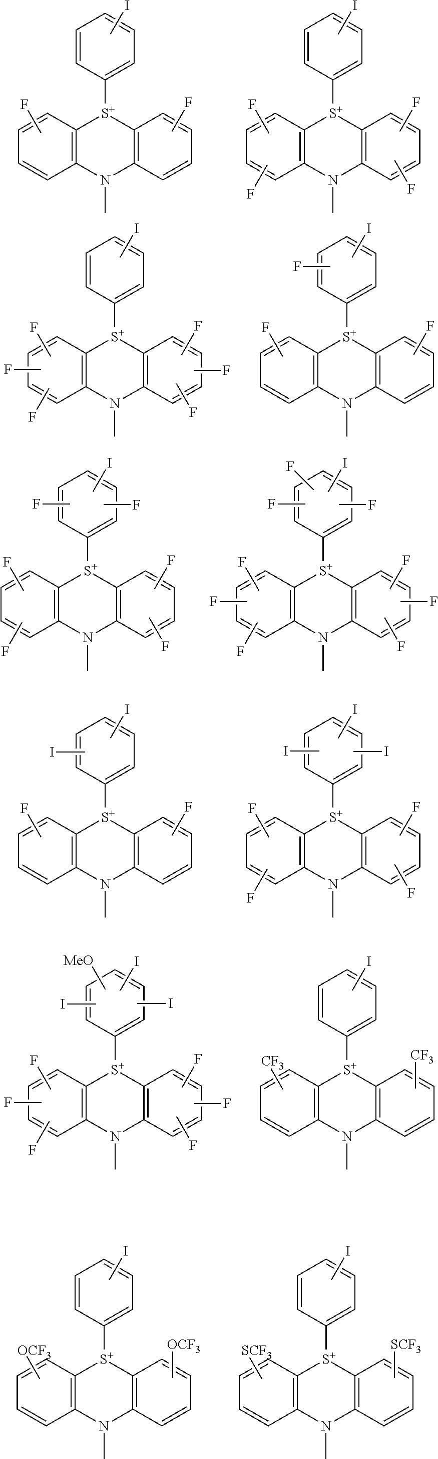

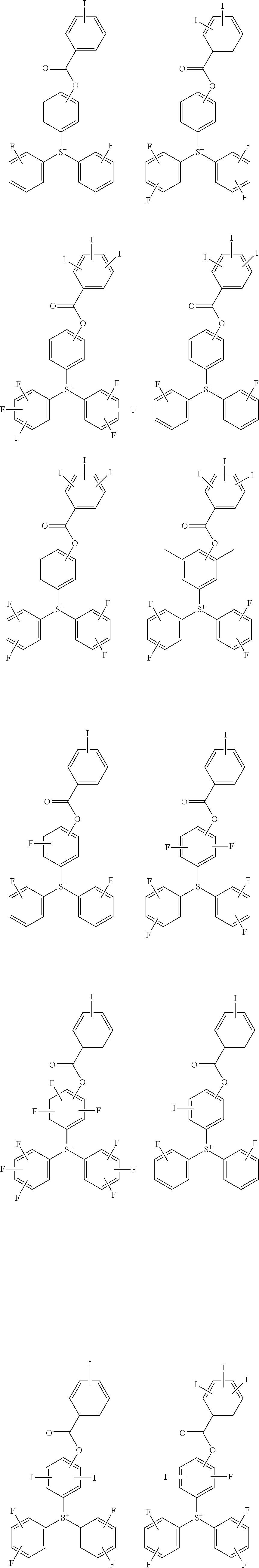

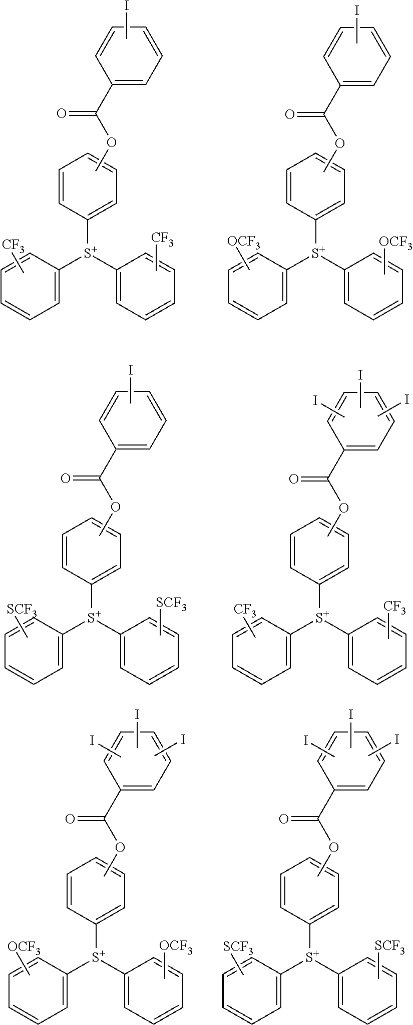

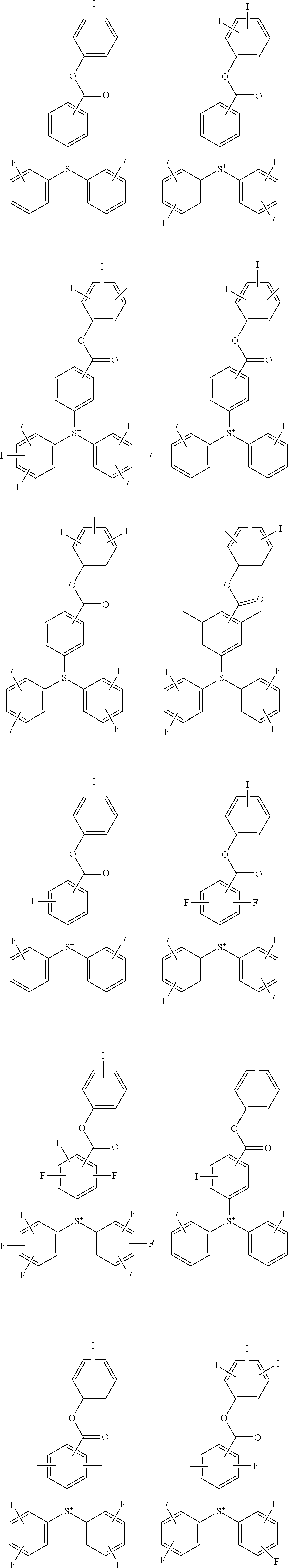

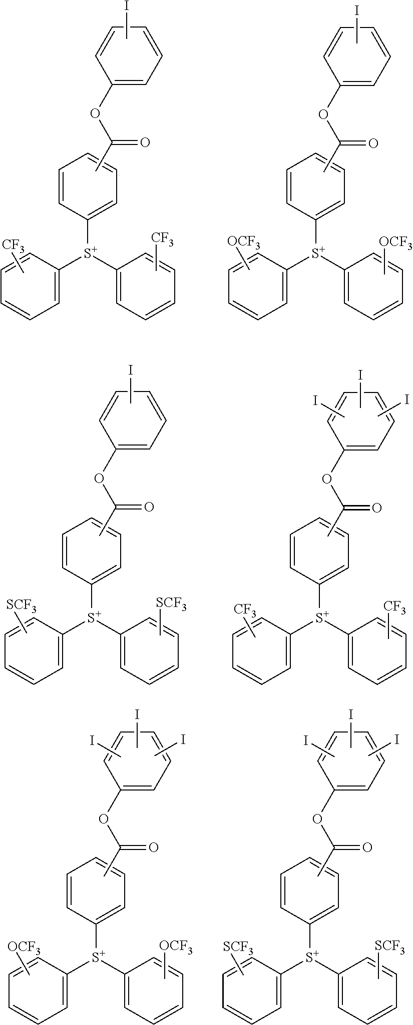

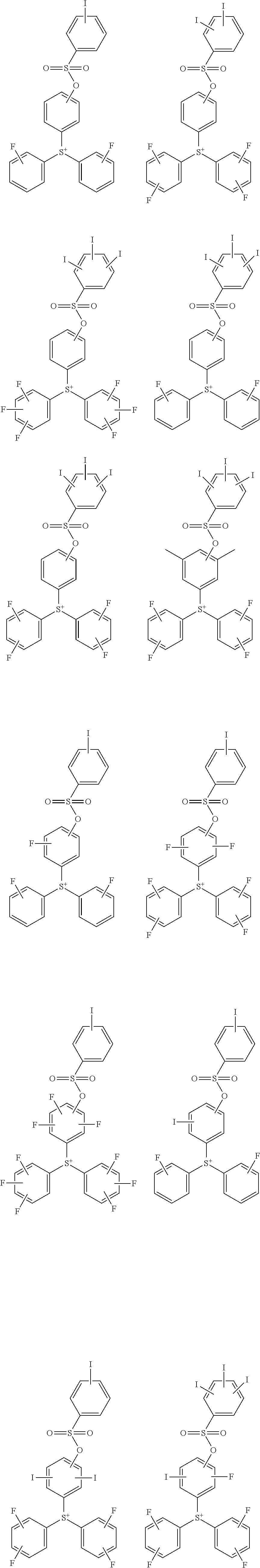

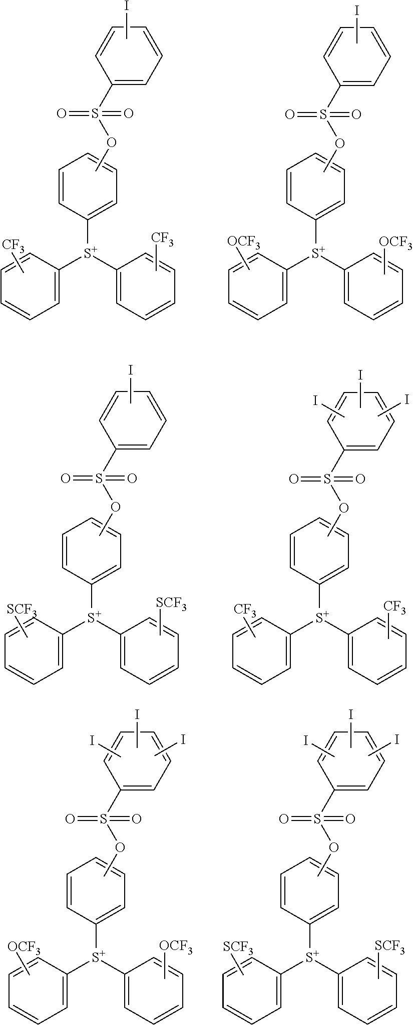

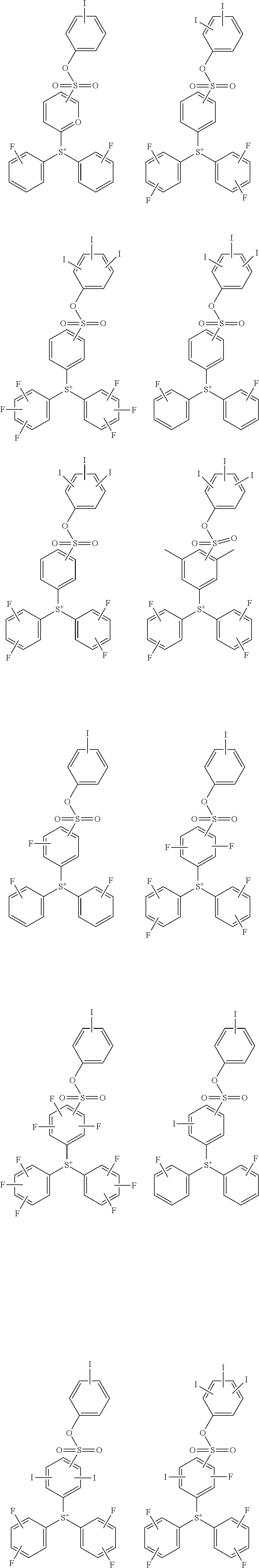

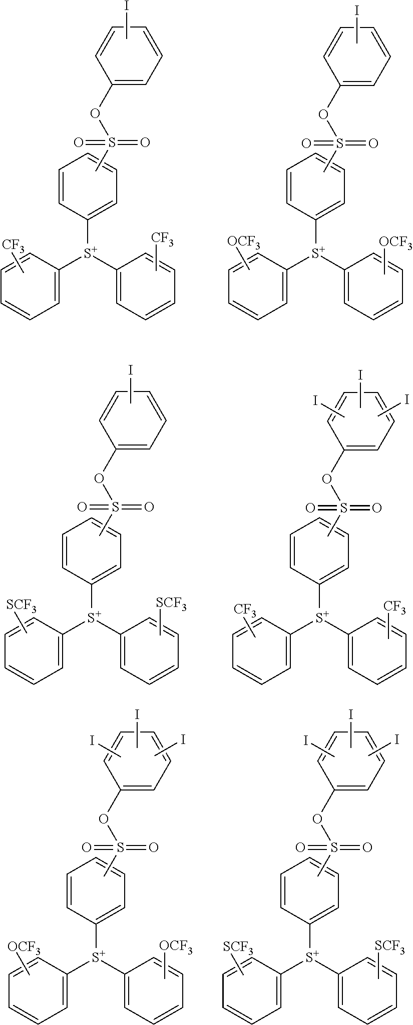

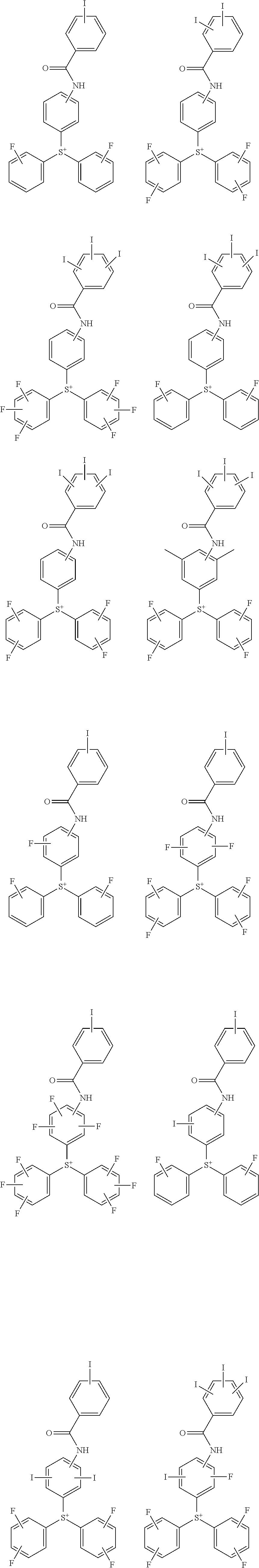

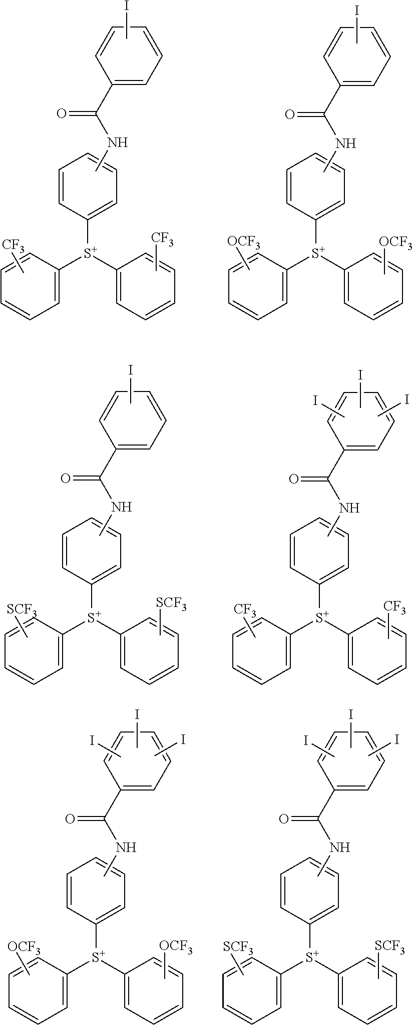

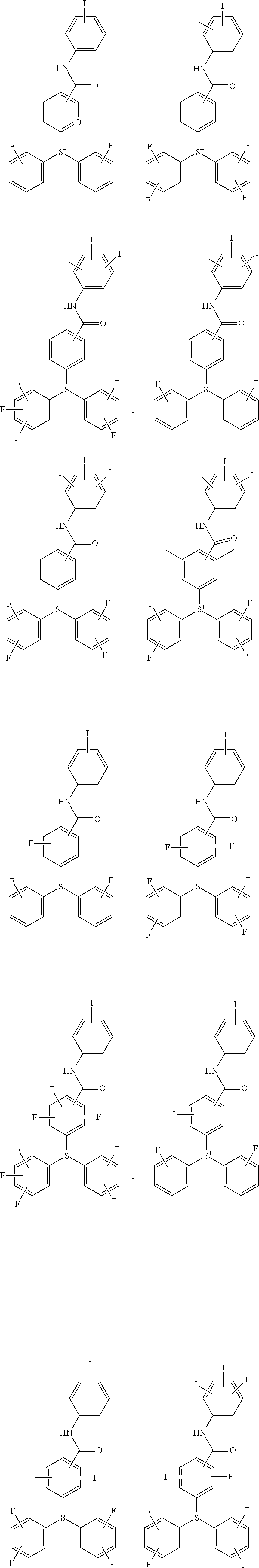

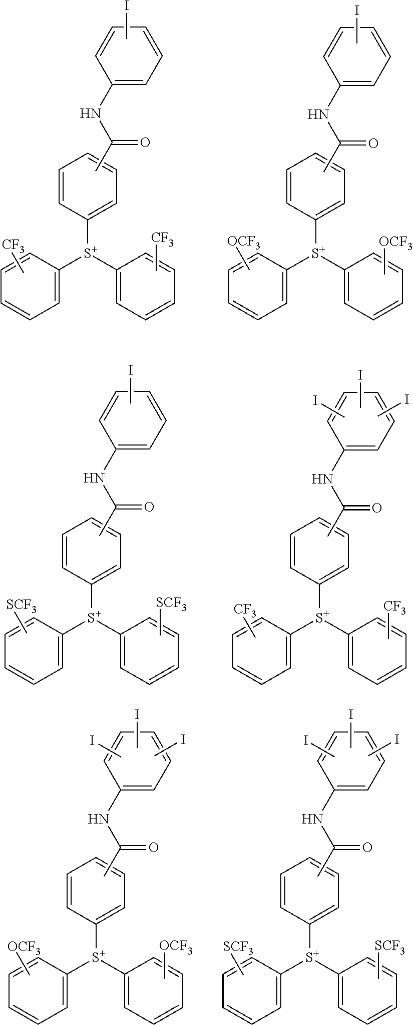

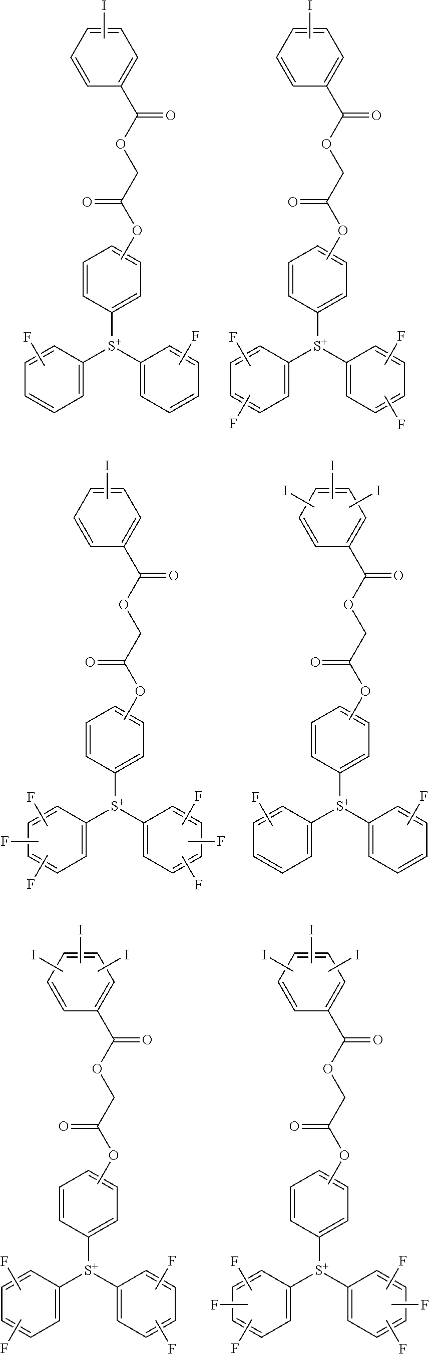

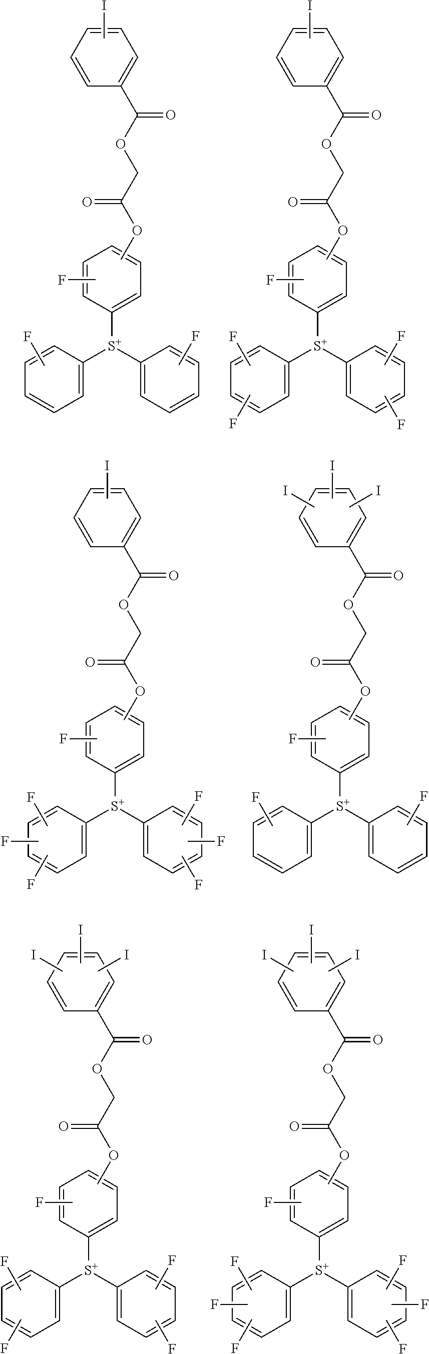

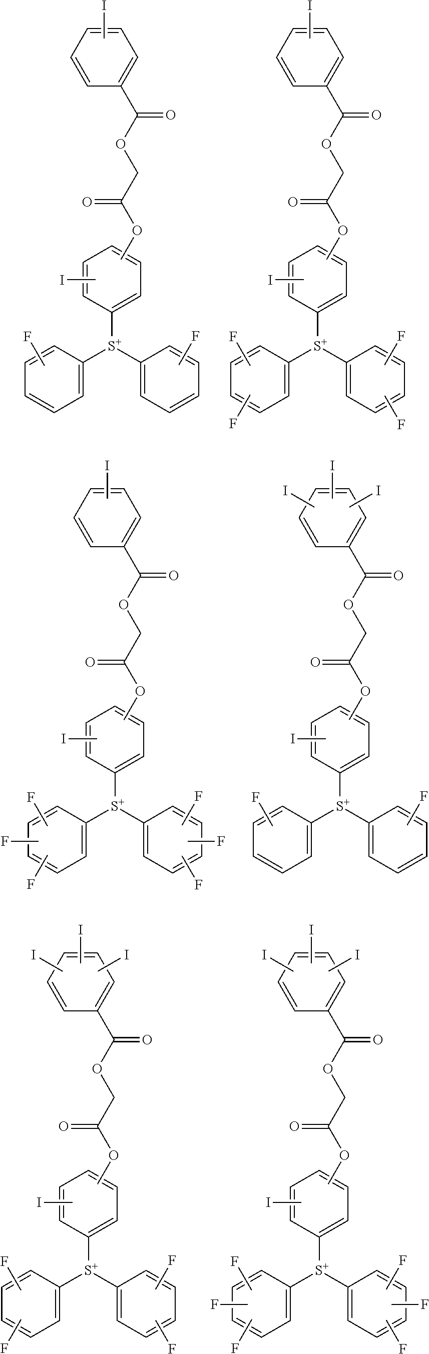

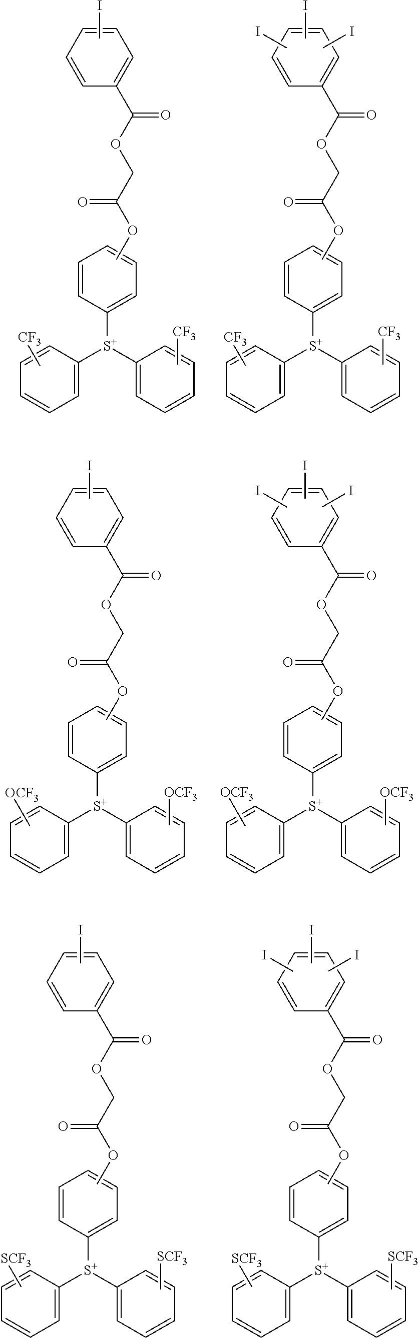

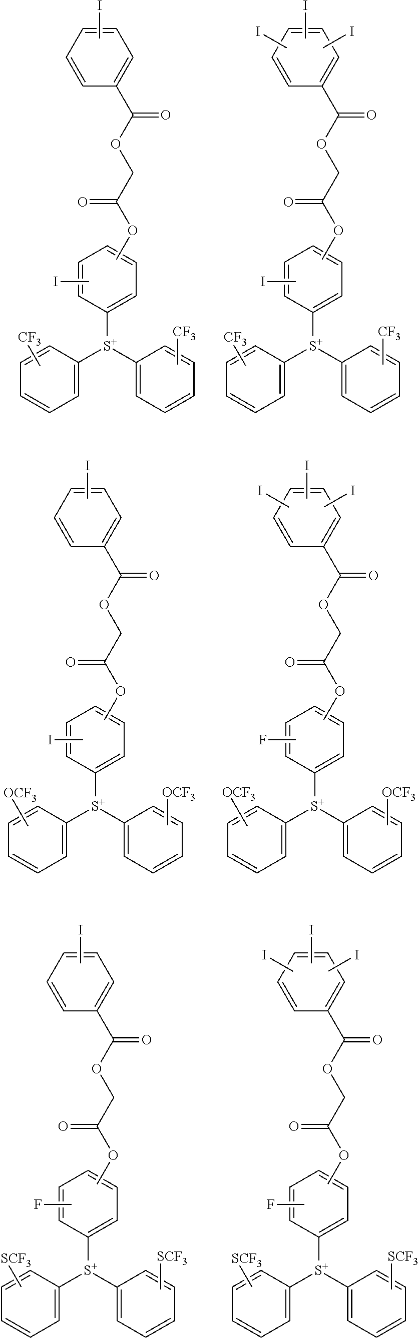

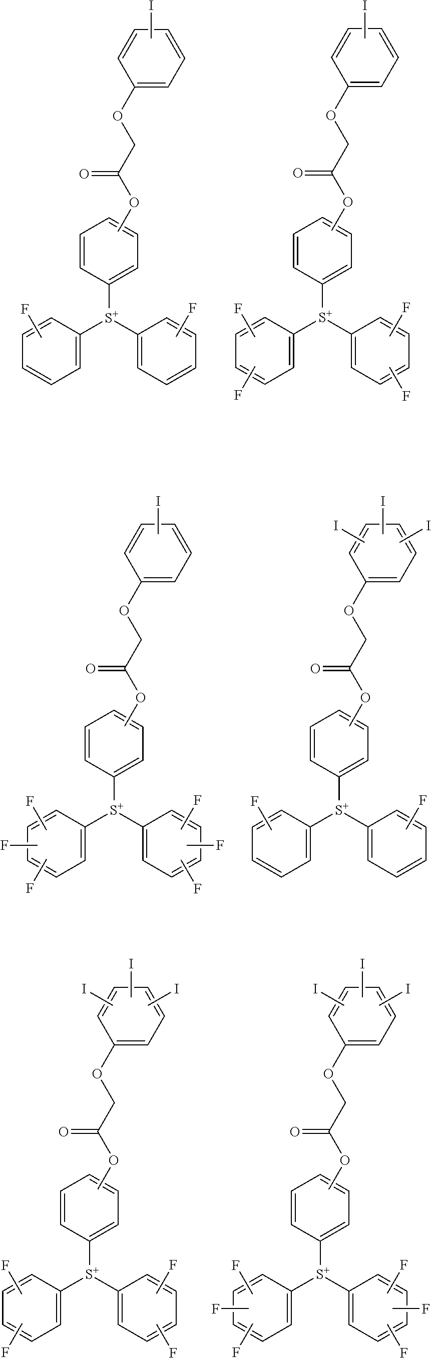

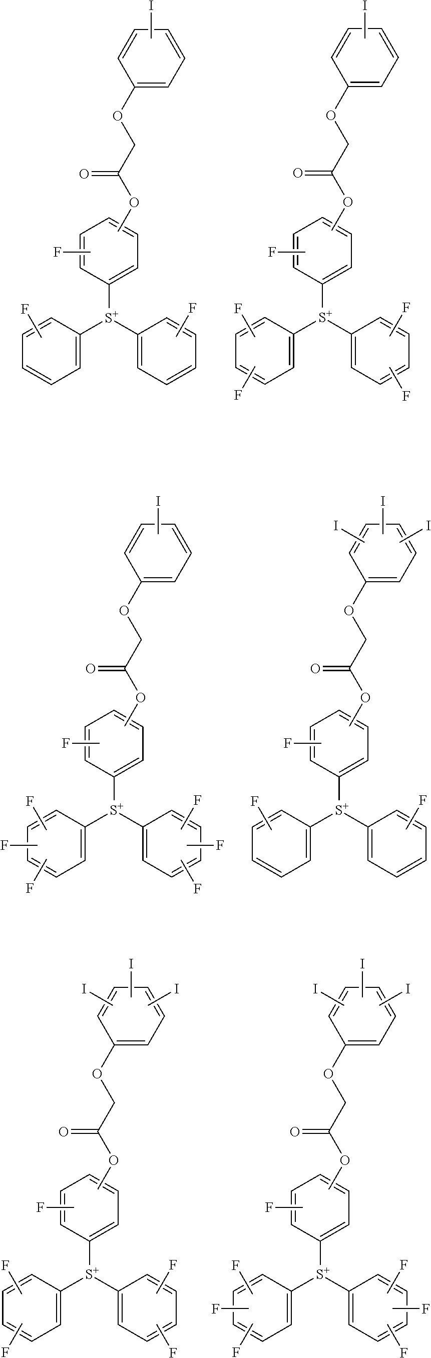

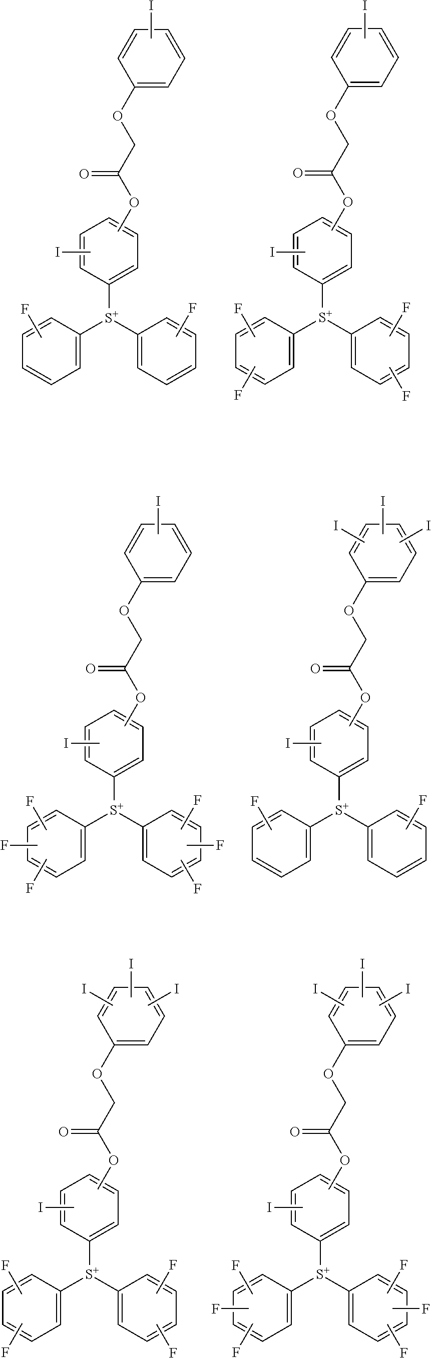

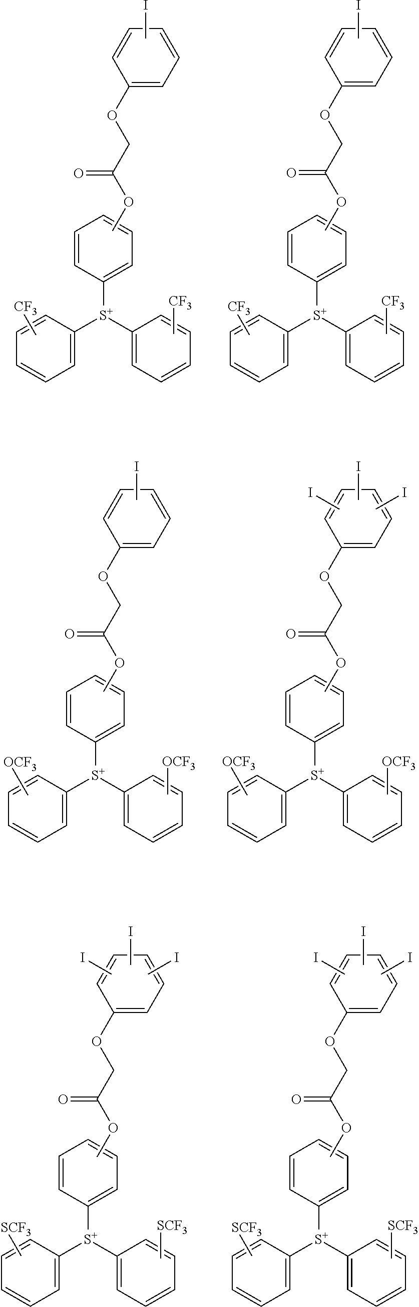

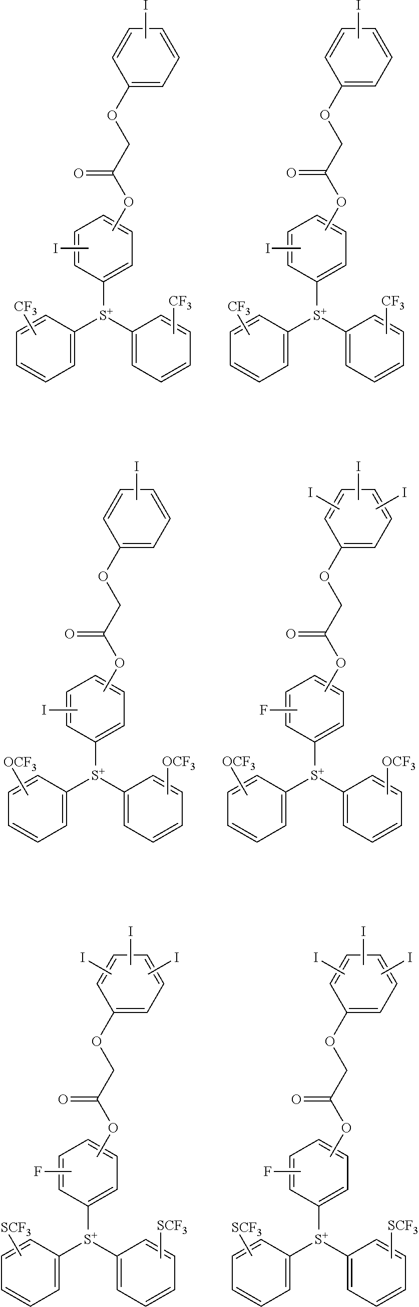

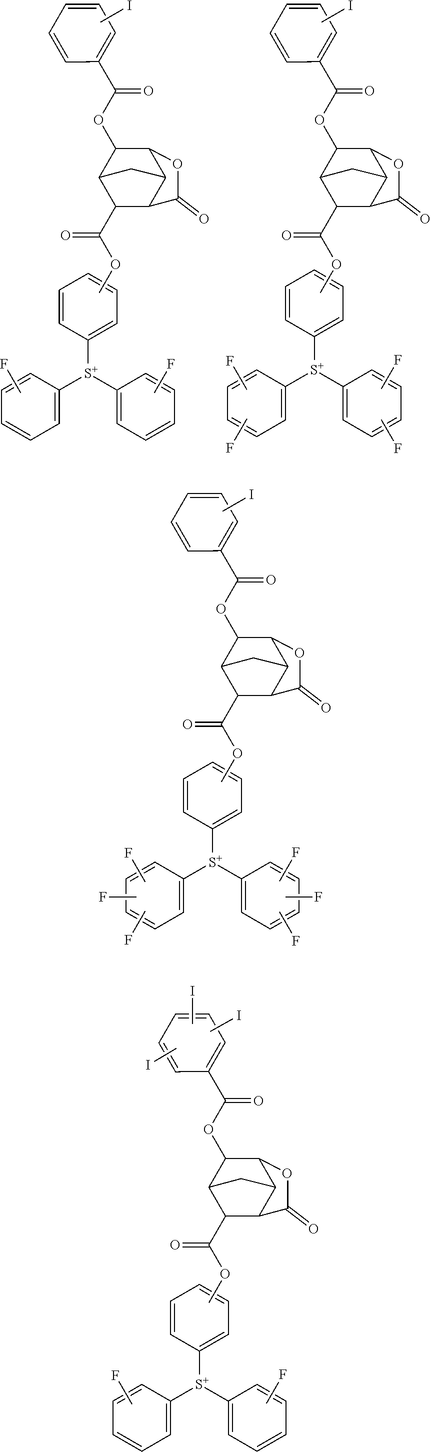

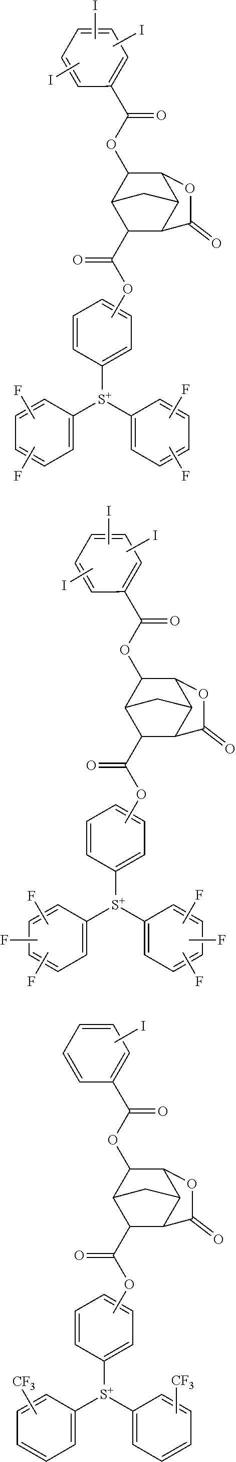

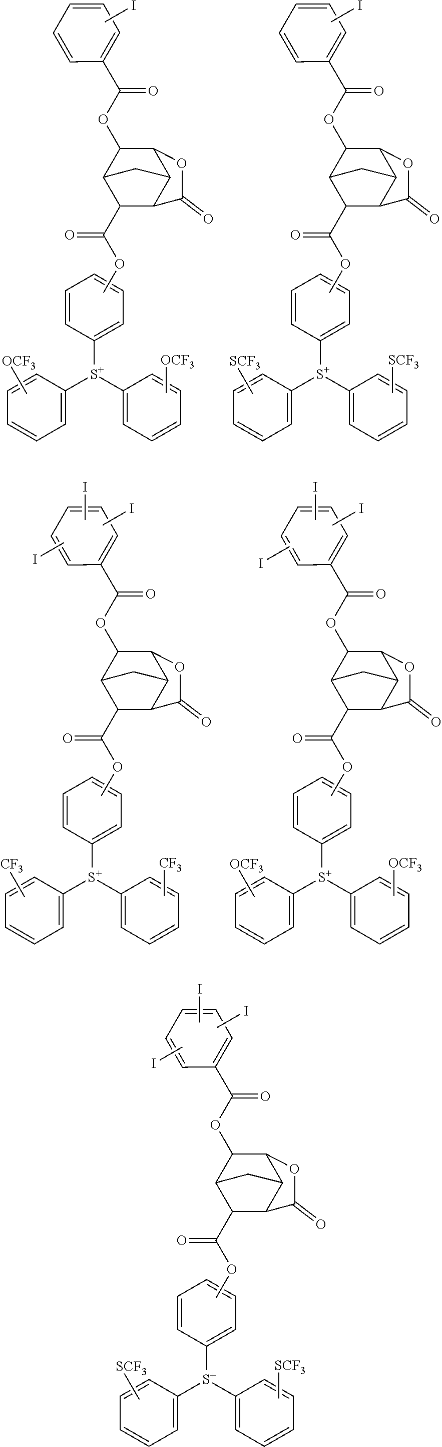

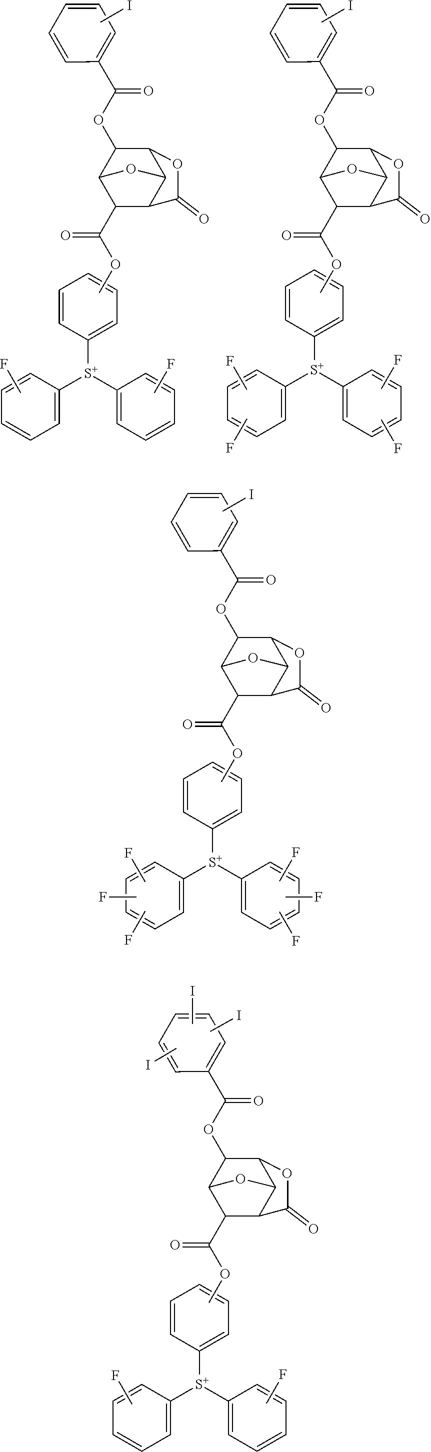

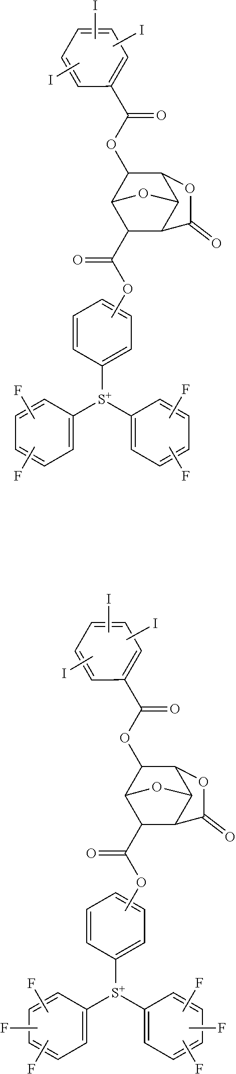

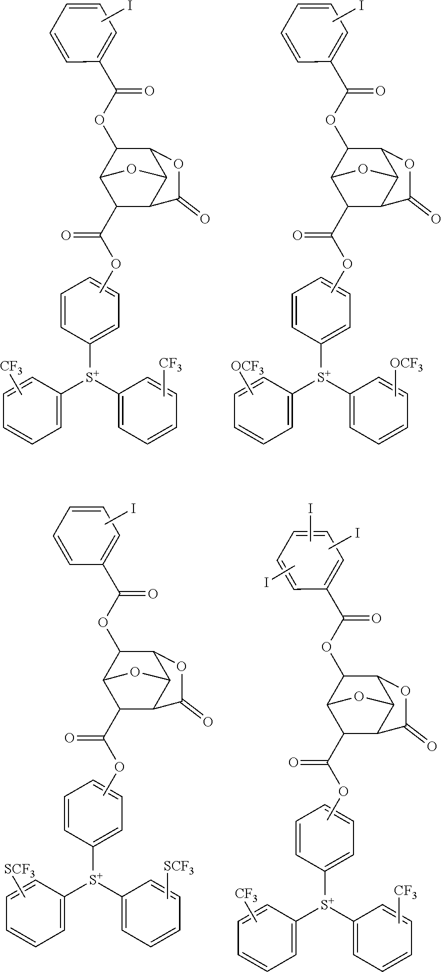

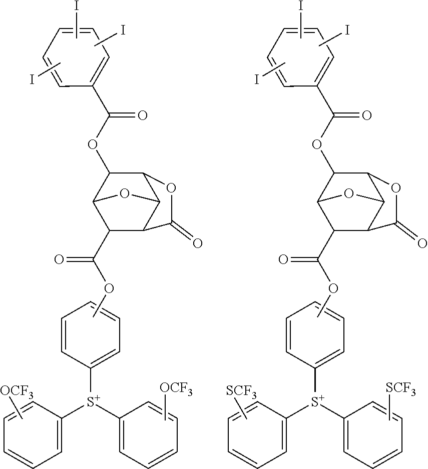

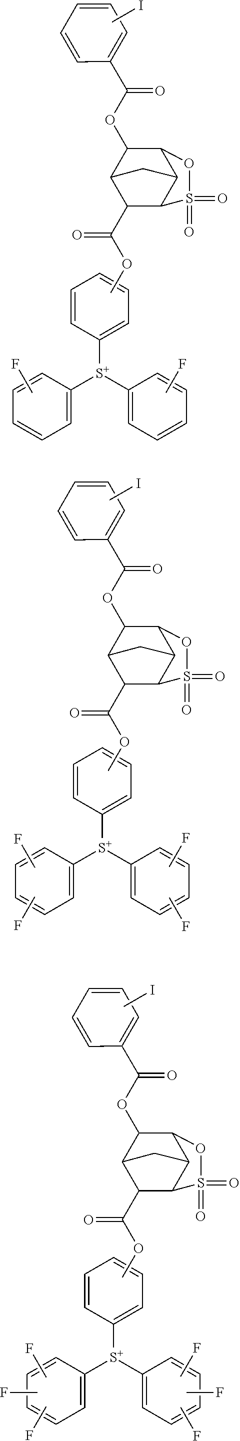

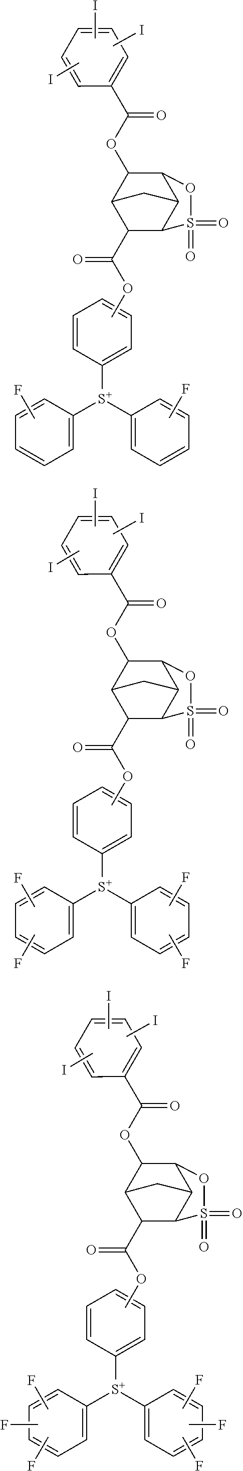

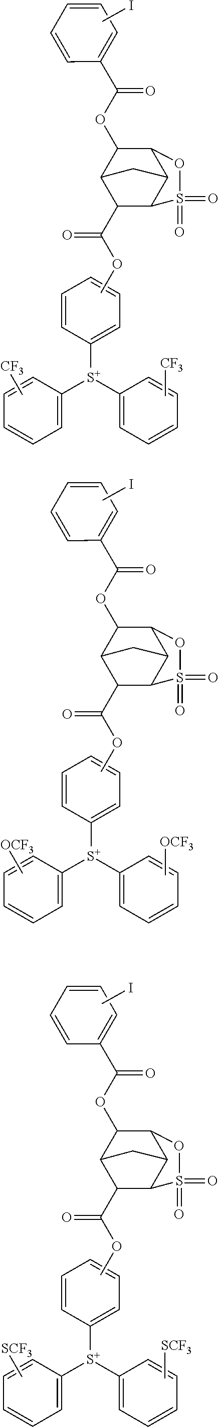

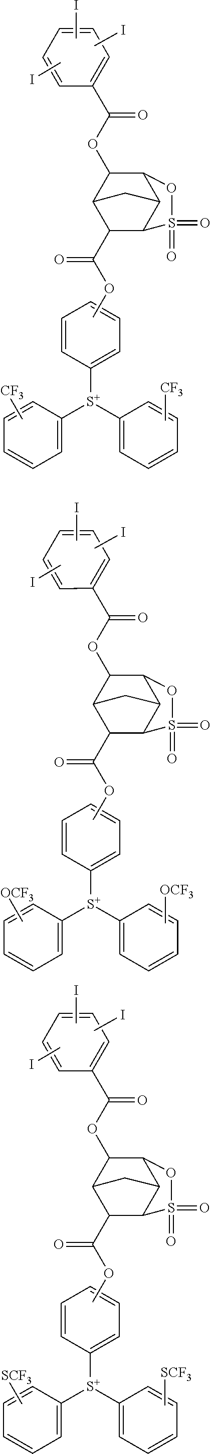

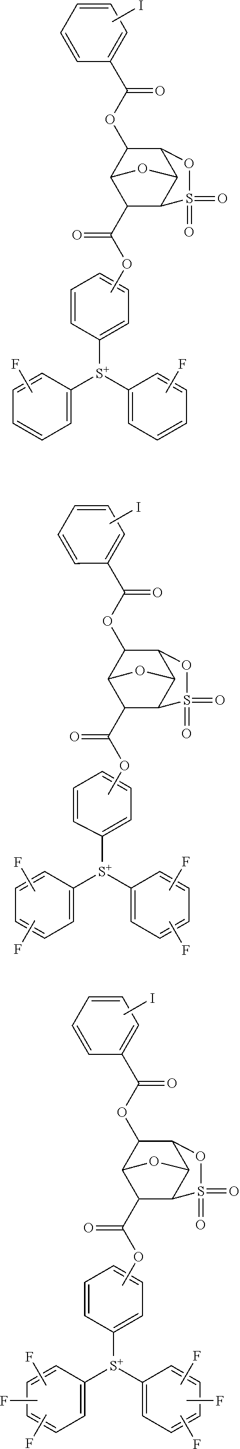

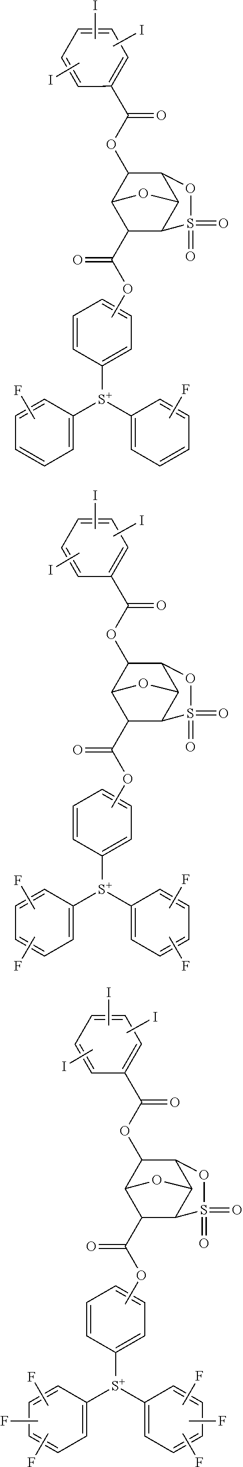

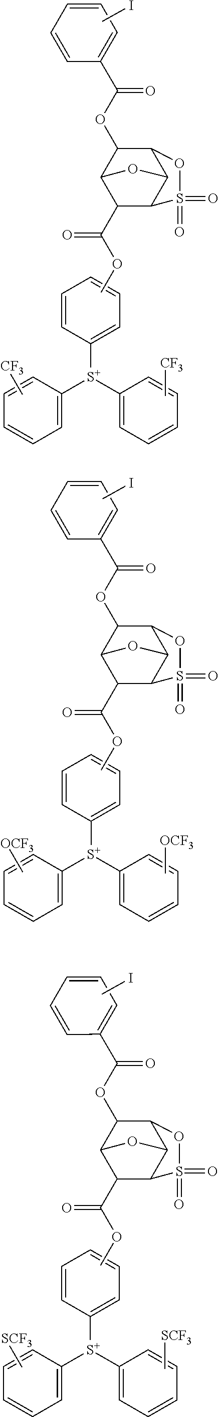

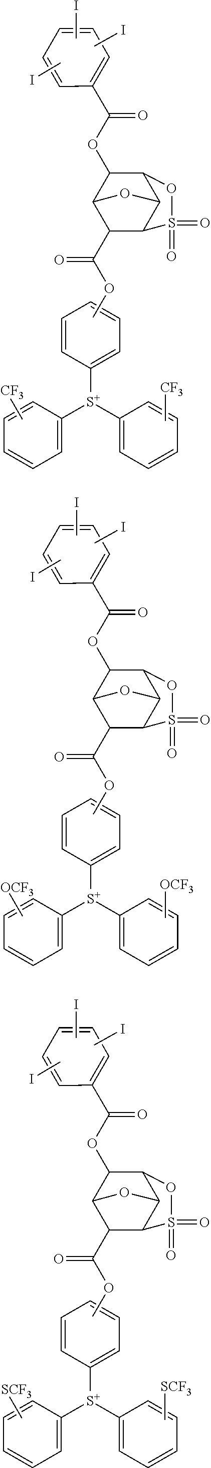

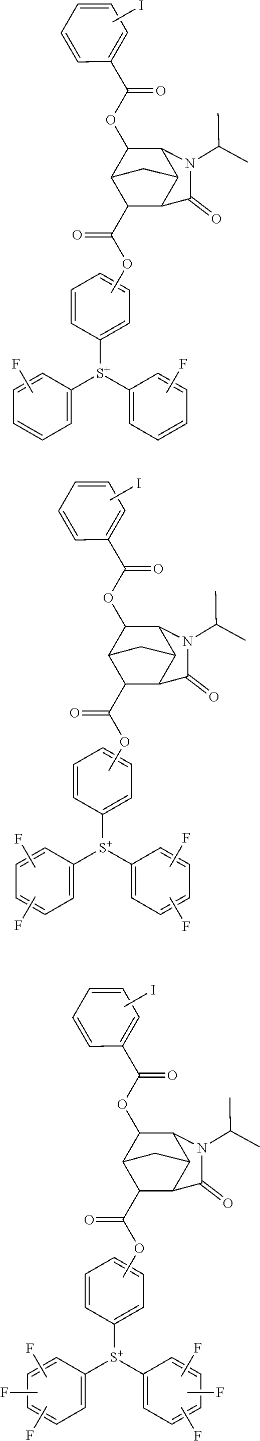

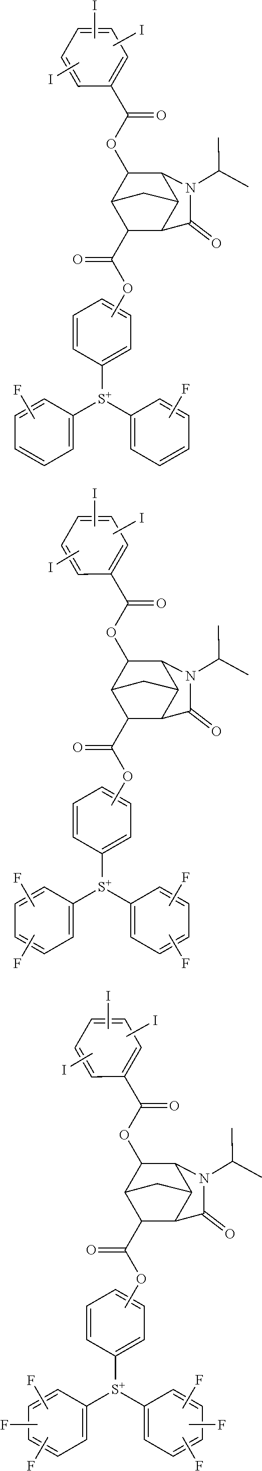

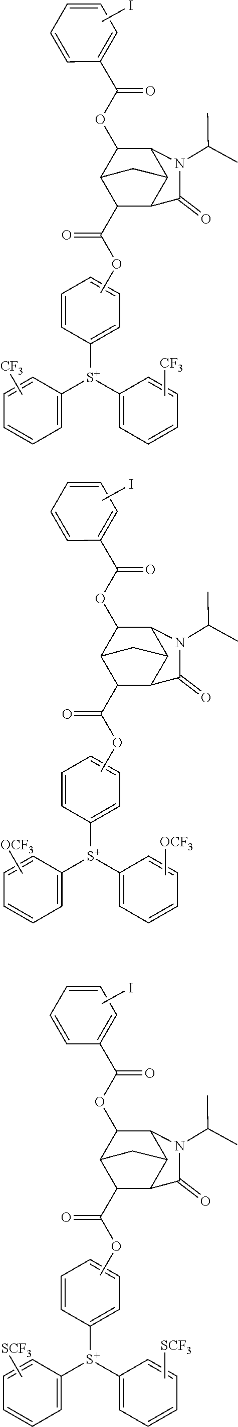

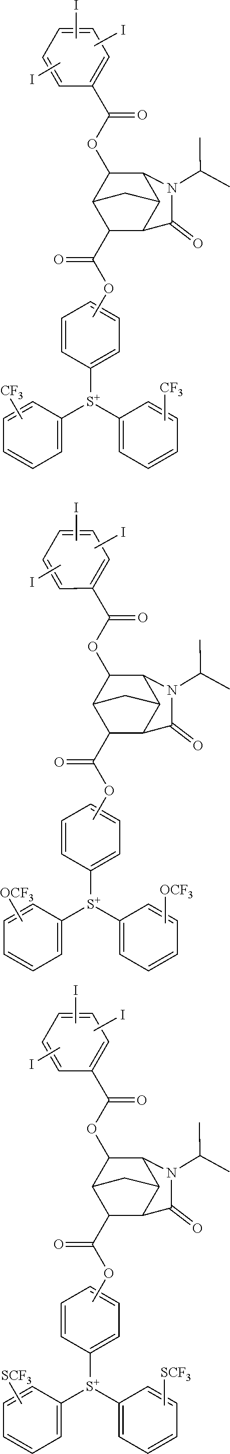

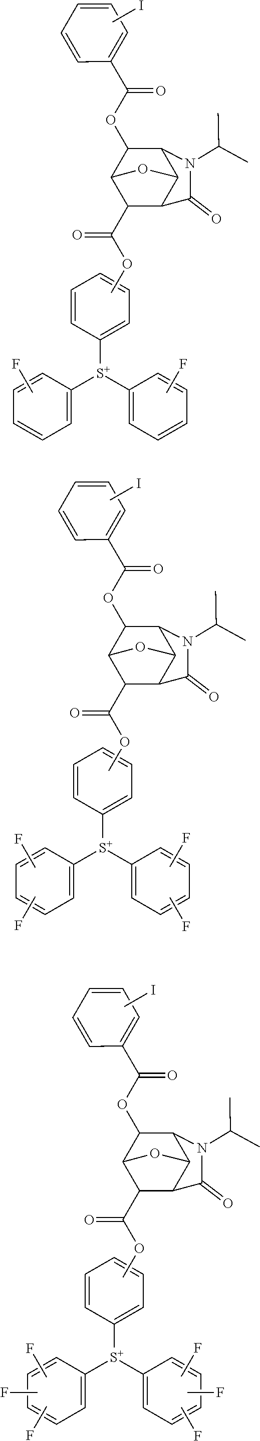

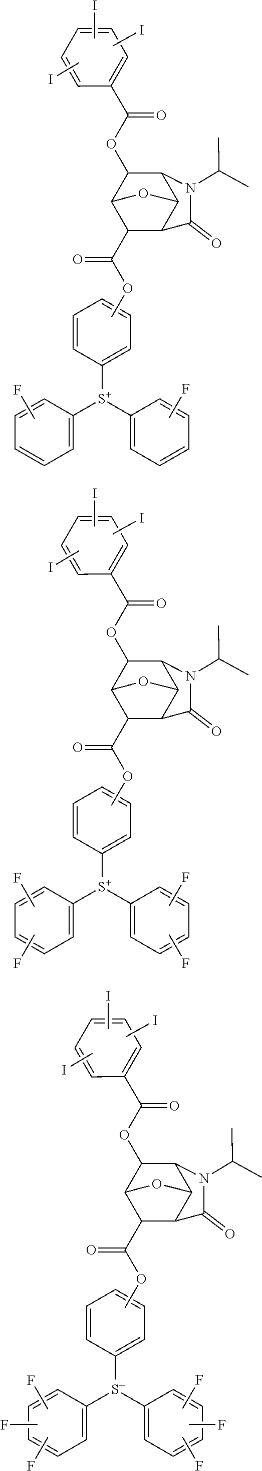

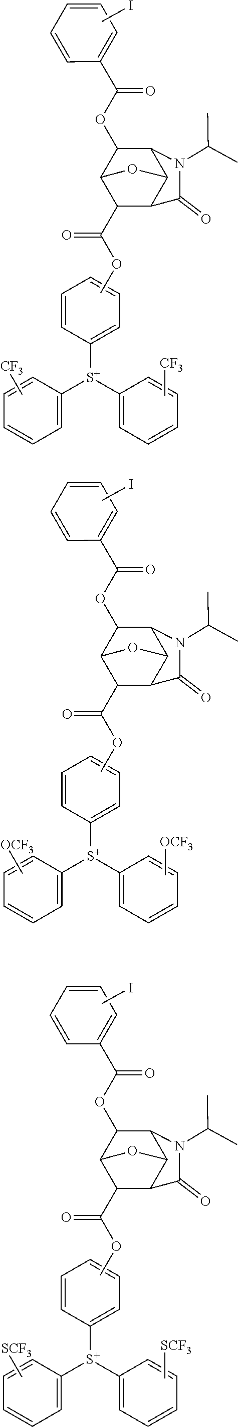

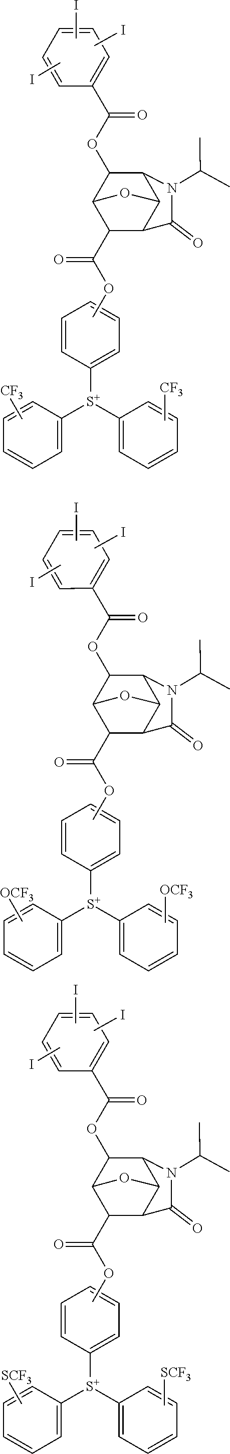

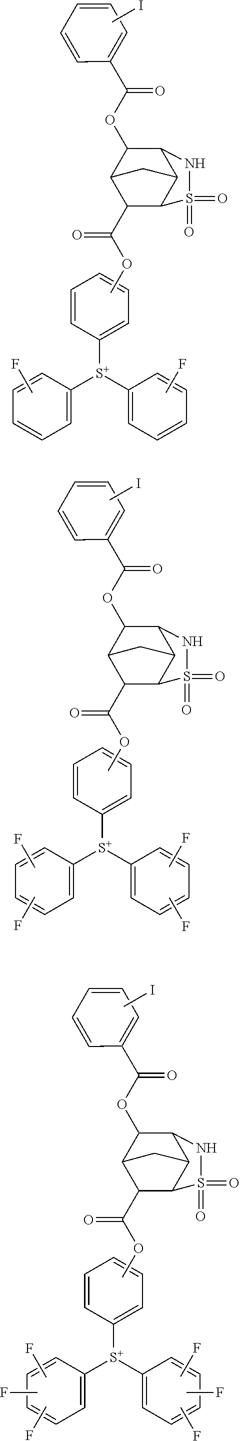

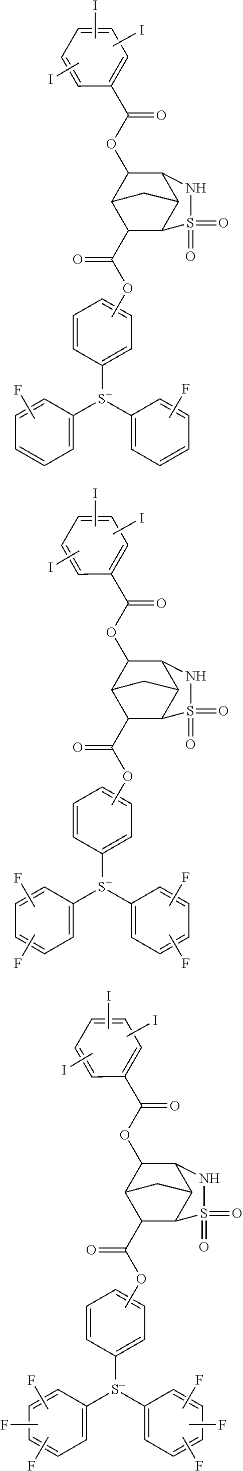

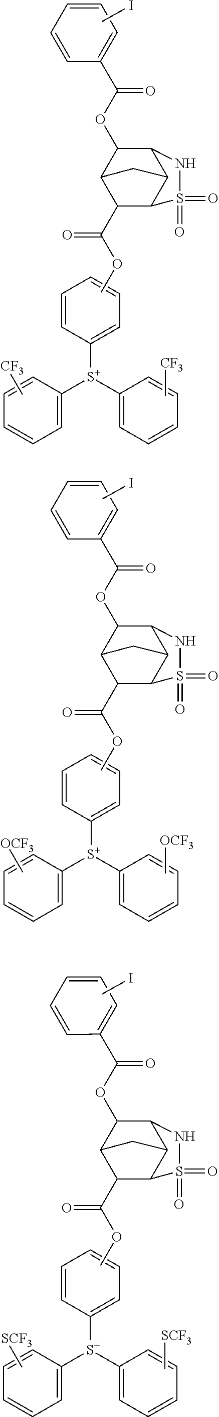

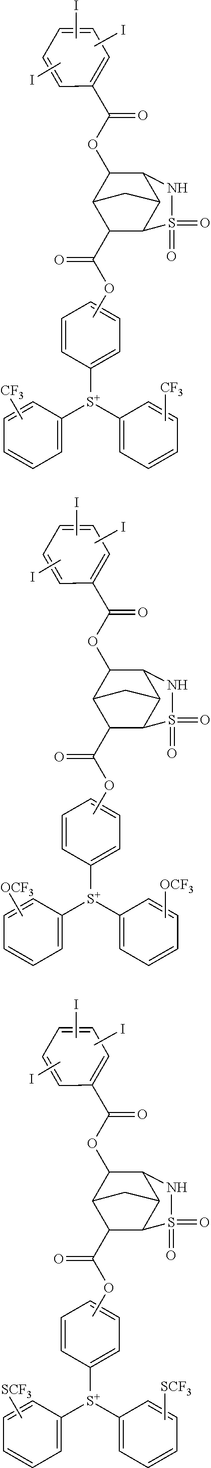

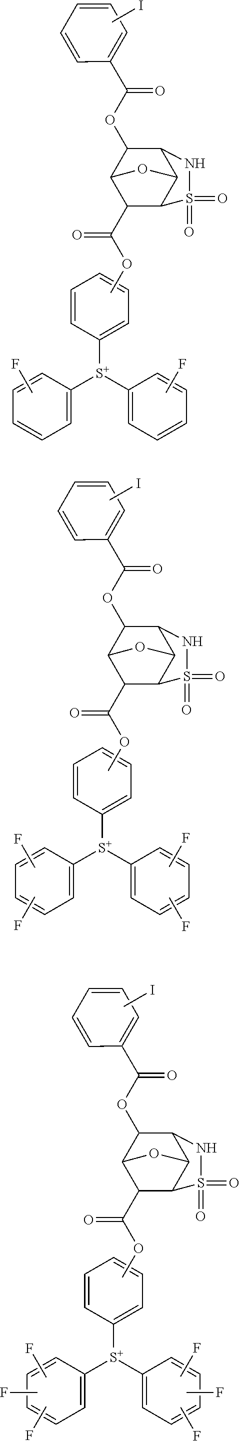

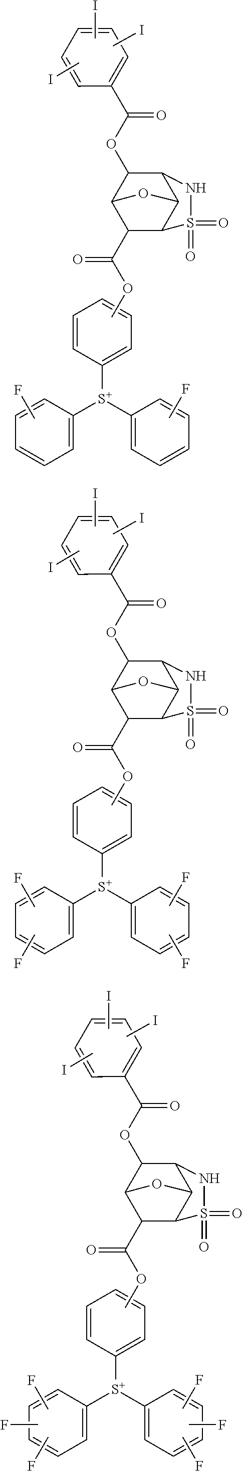

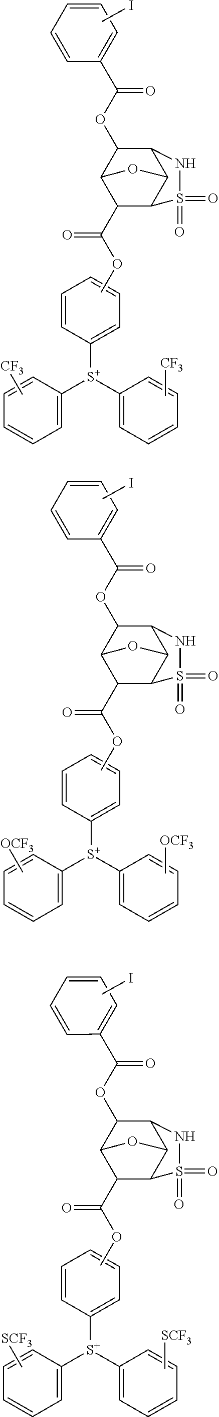

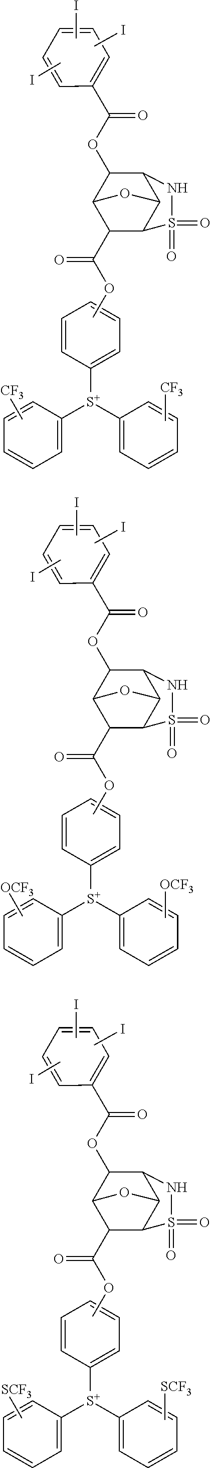

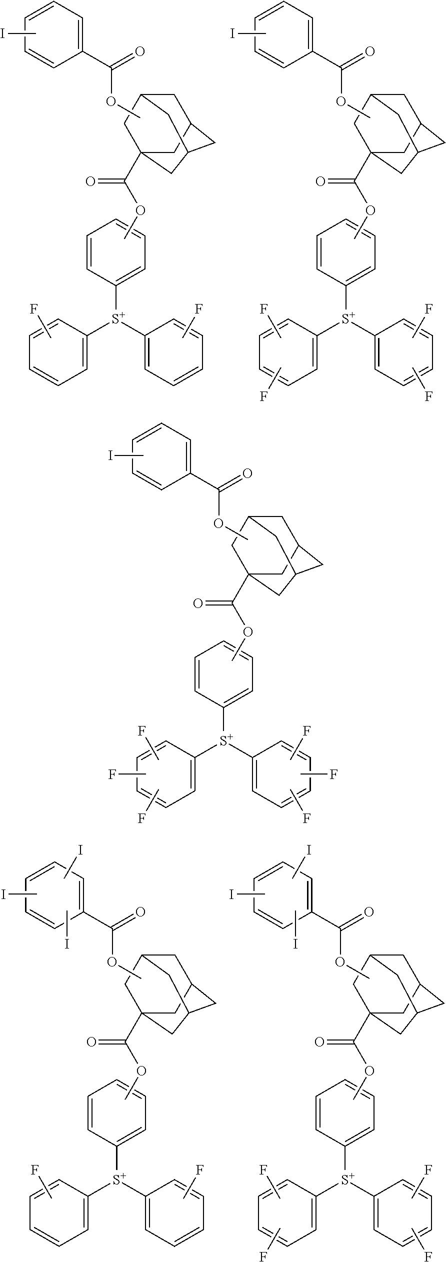

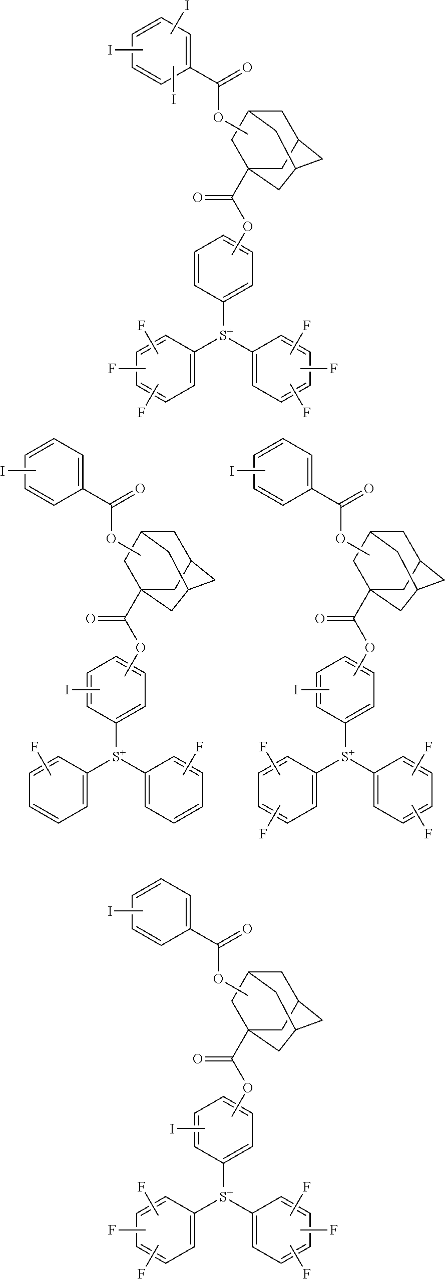

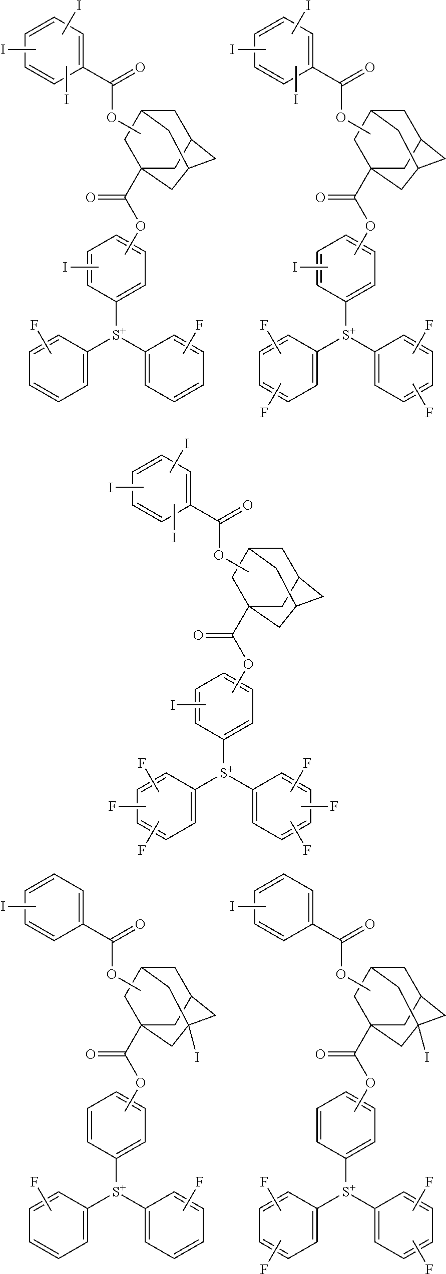

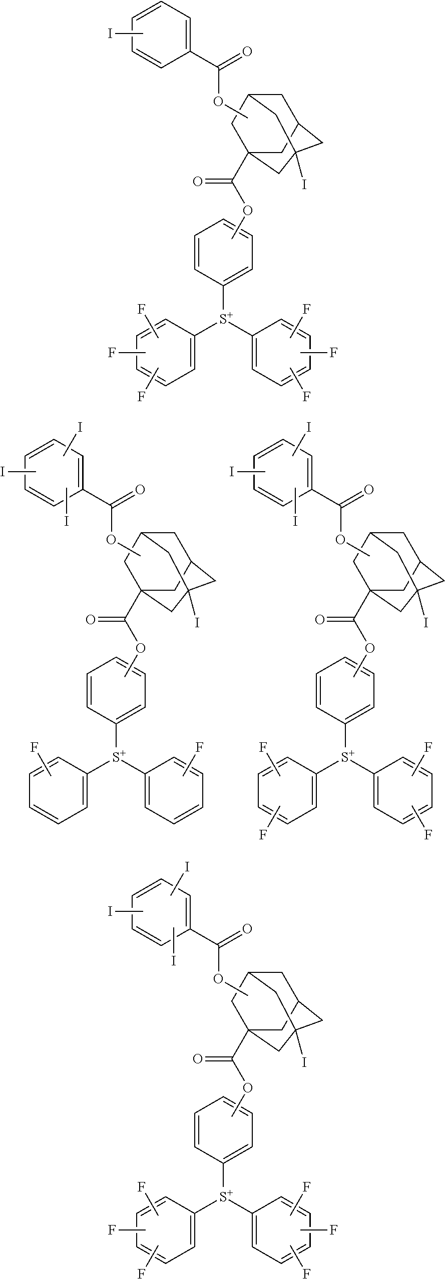

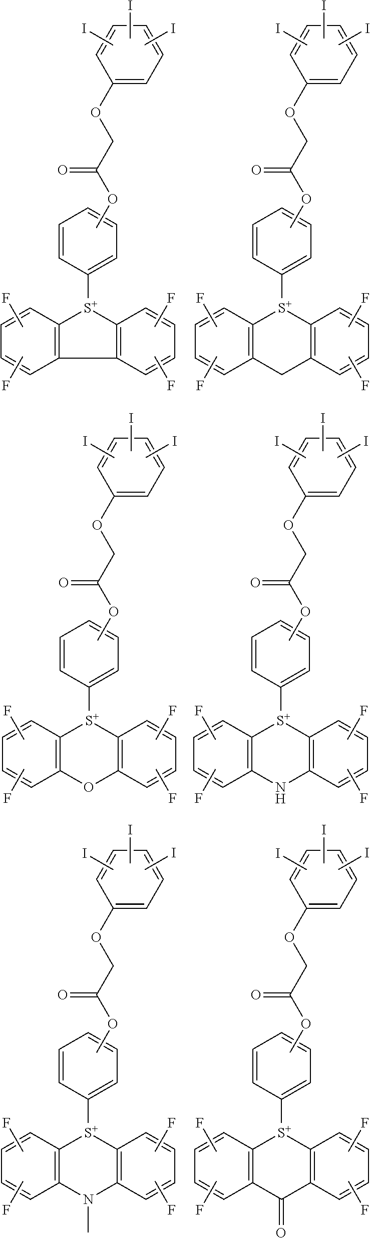

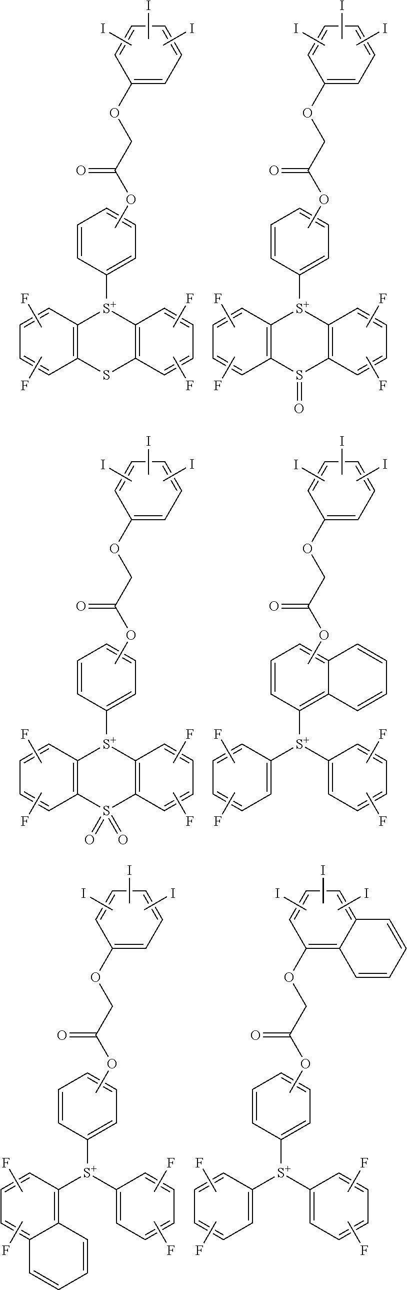

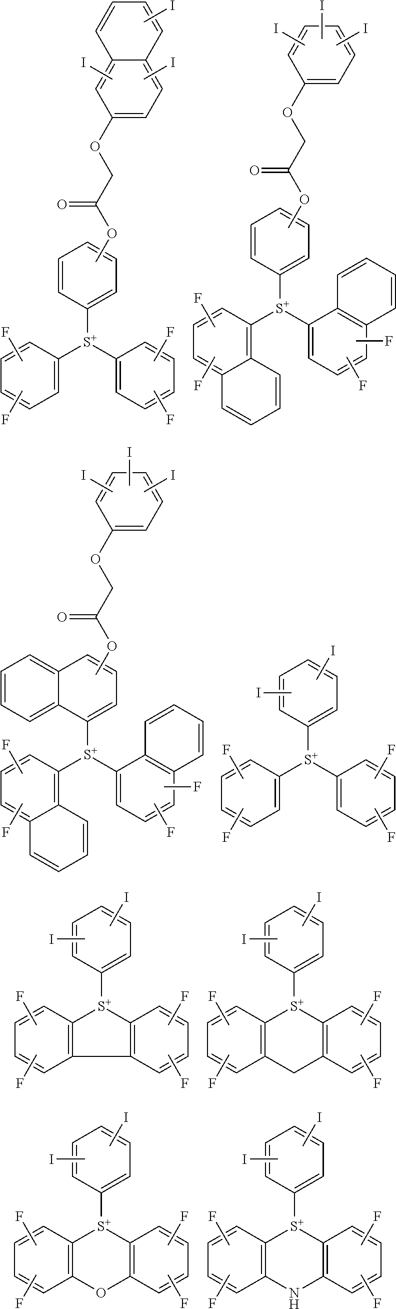

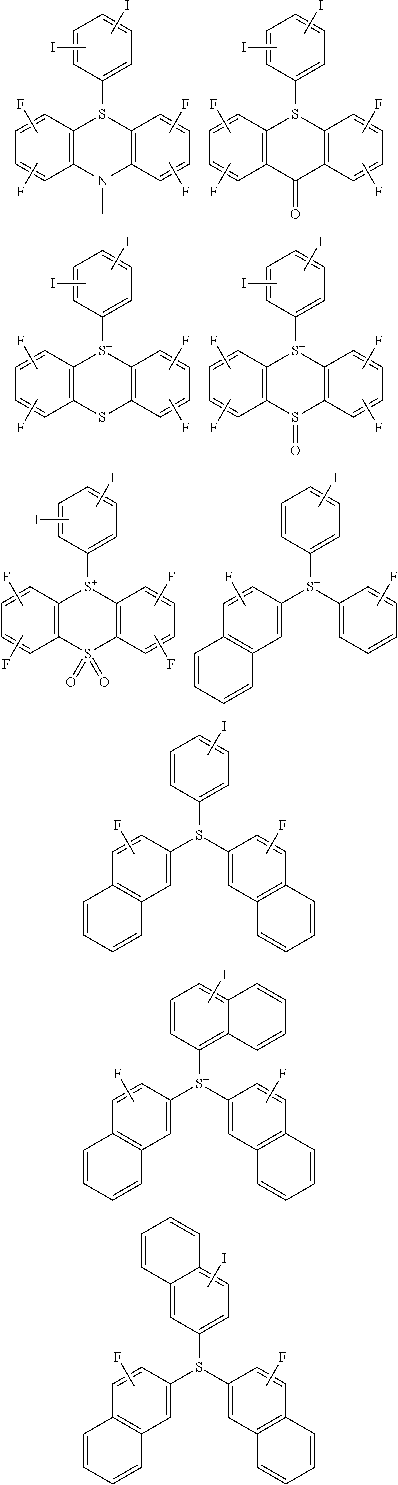

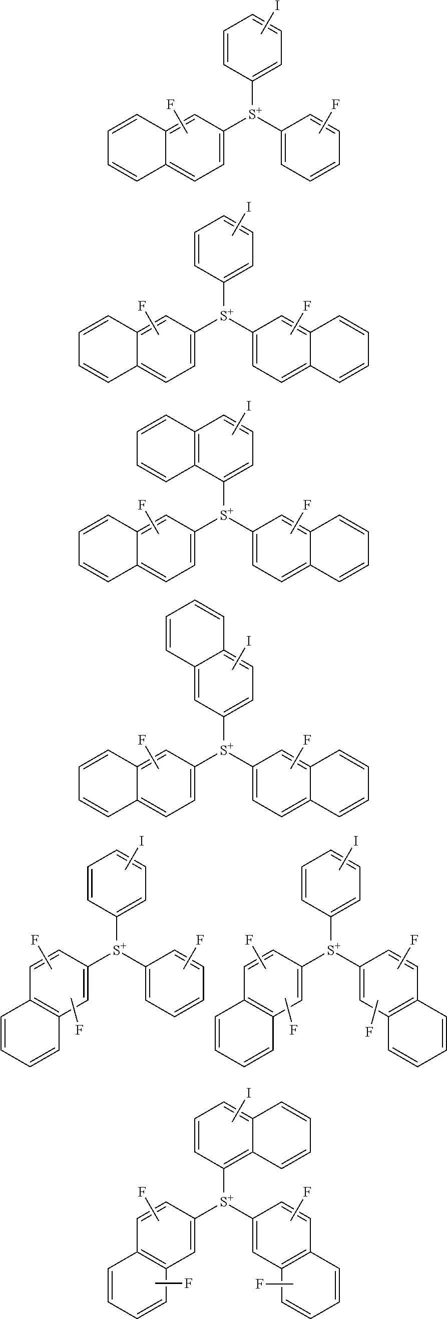

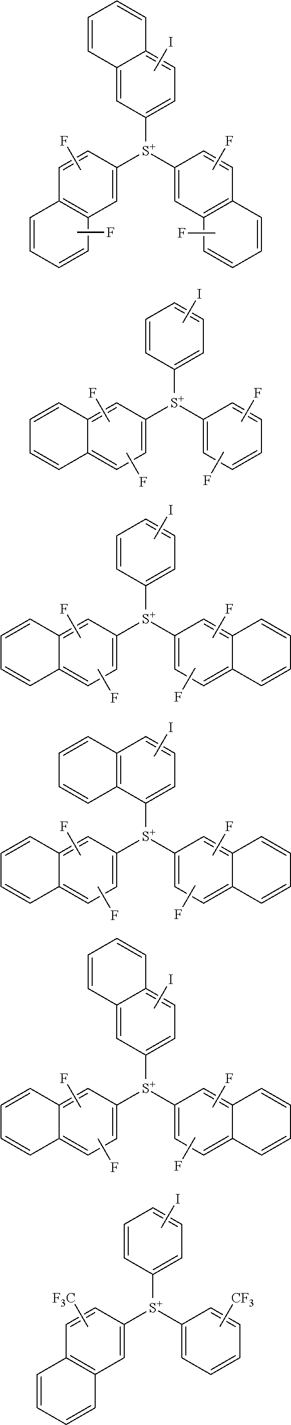

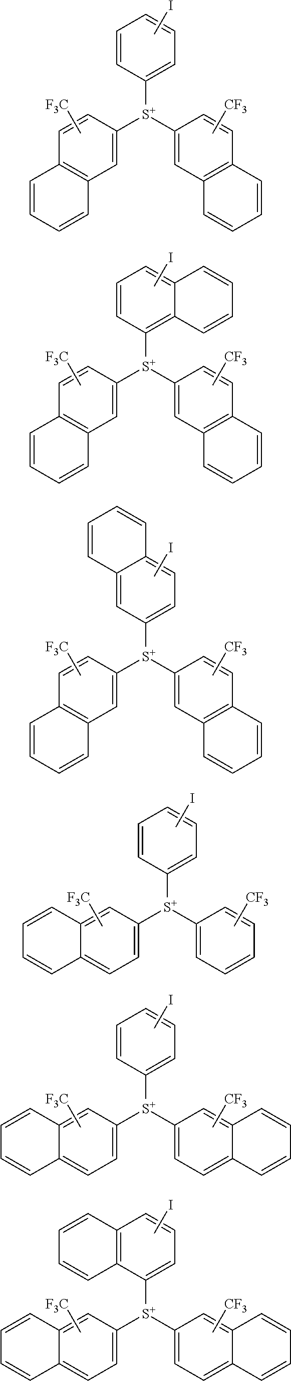

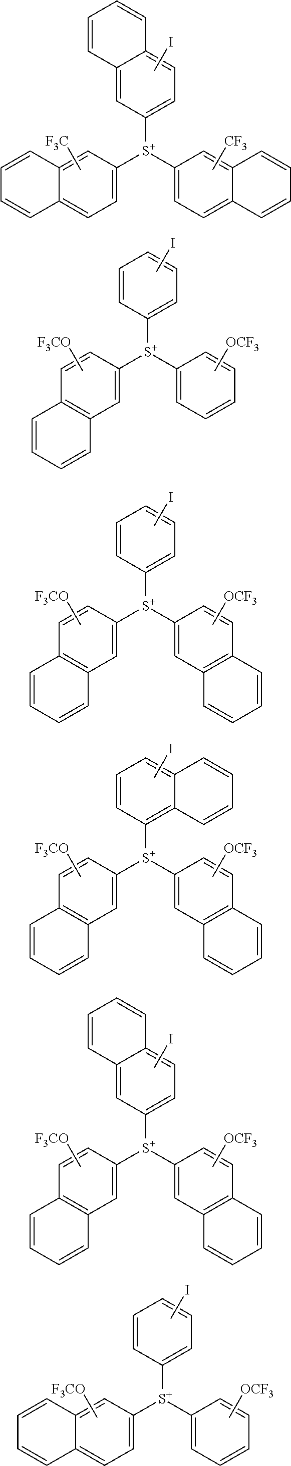

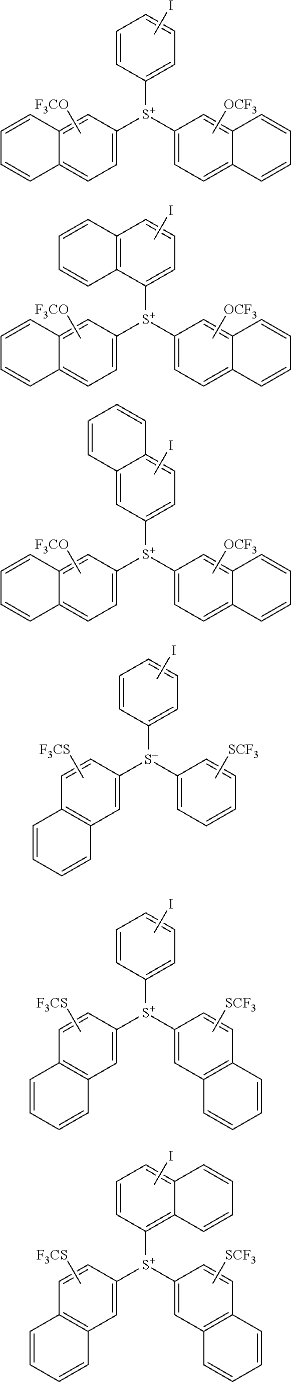

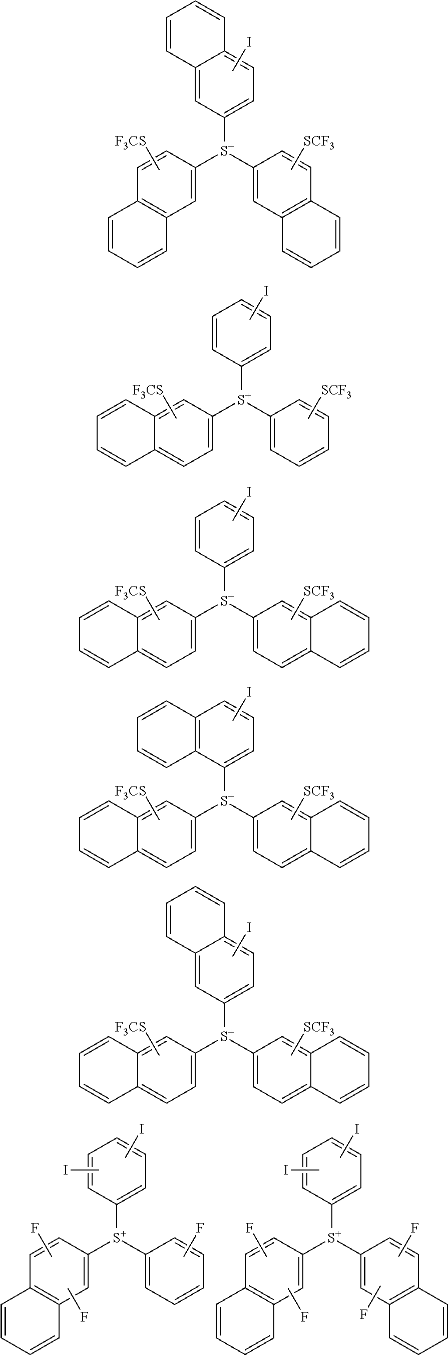

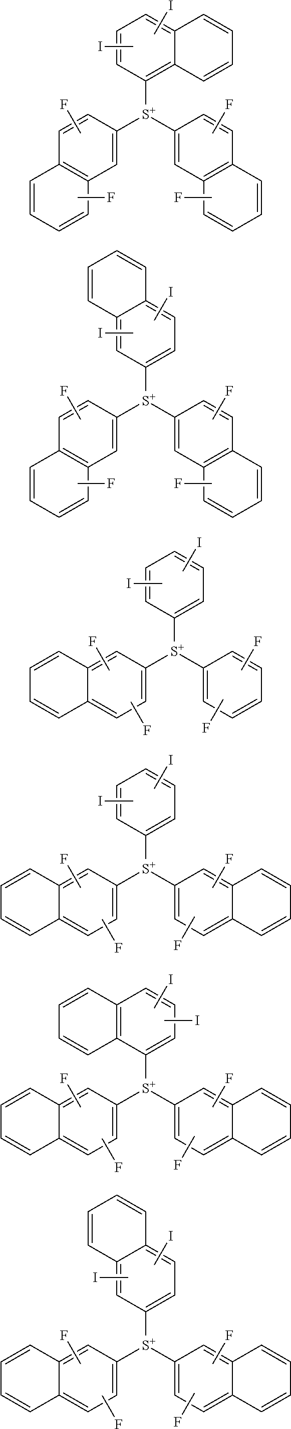

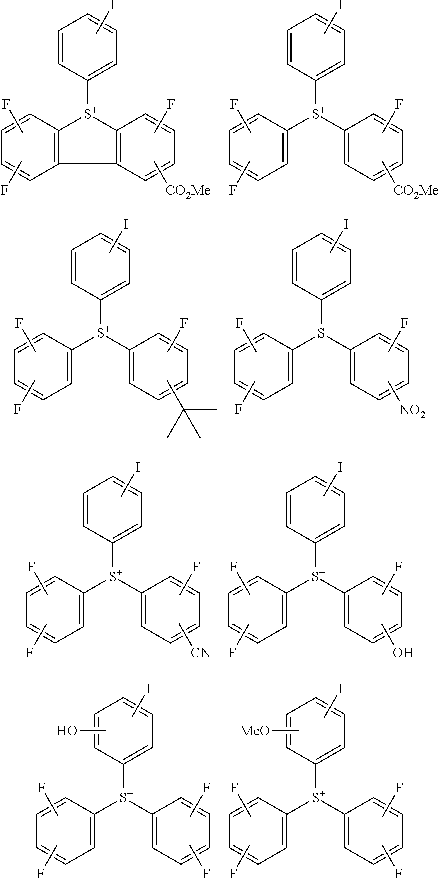

Examples of the iodonium cation include those described in JP-A 2024-000259, paragraph [0181], but are not limited thereto.

The ammonium cation preferably has the formula (am-1).

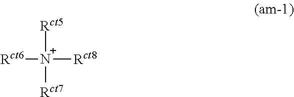

In formula (am-1), R^{ct5} to R^{ct8} are each independently a C₁-C₄₀ hydrocarbyl group which may contain a heteroatom. R^{ct5} and R^{ct6} may bond together to form a ring with the nitrogen atom to which they are attached. Examples of the hydrocarbyl group are as exemplified above for the hydrocarbyl group R¹ to R³ in formula (1).

Examples of the ammonium cation having formula (am-1) are shown below, but not limited thereto.

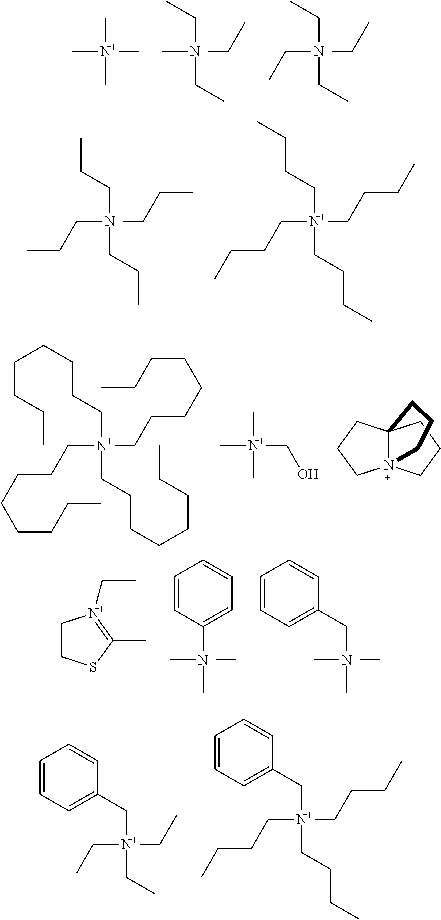

Examples of the repeat units (c1) to (c5) include arbitrary combinations of anions with cations, both as exemplified above.

Of the repeat units (c1) to (c5), repeat units (c2) to (c5) are preferred from the aspect of controlling acid diffusion, repeat units (c2), (c4) and (c5) are more preferred from the aspect of the acid strength of generated acid, and repeat units (c2) are most preferred from the aspect of solvent solubility.

The polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

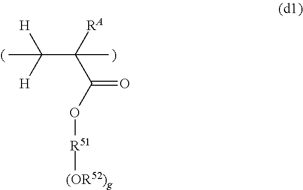

In formula (d1), R^{A} is hydrogen, fluorine, methyl or trifluoromethyl. R⁵¹ is a C₁-C₃₀ (g+1)-valent hydrocarbon group which may contain a heteroatom. R⁵² is an acid labile group, and g is 1, 2, 3 or 4.

In formula (d1), the acid labile group R⁵² is deprotected under the action of acid so that a hydroxy group is generated. Although the structure of R⁵² is not particularly limited, an acetal structure, ketal structure, hydrocarbyloxycarbonyl group and hydrocarbyloxymethyl group having the following formula (d2) are preferred, with the hydrocarbyloxymethyl group having formula (d2) being more preferred.

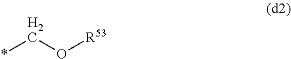

Herein R⁵³ is a C₁-C₁₅ hydrocarbyl group.

Illustrative examples of the acid labile group R⁵², the hydrocarbyloxymethyl group having formula (d2), and the repeat units (d) are as exemplified for the repeat units (d) in JP-A 2020-111564 (U.S. Pat. No. 11,560,355).

In addition to the foregoing units, the polymer may further comprise repeat units (e) derived from indene, benzofuran, benzothiophene, acenaphthylene, chromone, coumarin, norbornadiene and derivatives thereof. Examples of the monomer from which repeat units (e) are derived are shown below, but not limited thereto.

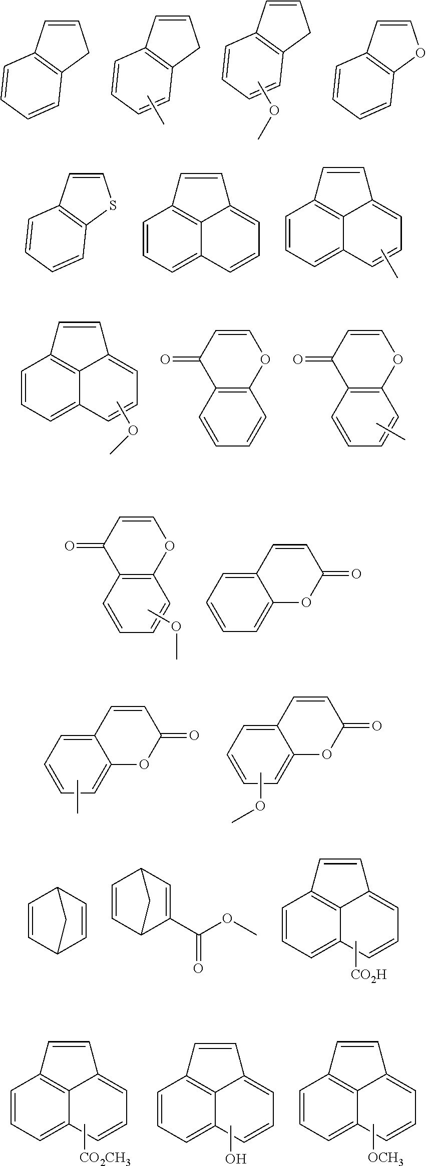

The polymer may further comprise repeat units (f) derived from indane, vinylpyridine or vinylcarbazole.

In the polymer, repeat units (a1), (a2), (a3), (b1), (b2), (c1) to (c5), (d), (e), and (f) are incorporated in a ratio of preferably 0≤a1≤0.8, 0≤a2≤0.8, 0≤a3≤0.6, 0≤b1≤0.8, 0≤b2≤0.5, 0≤c1≤0.4, 0≤c2≤0.4, 0≤c3≤0.4, 0≤c4≤0.4, 0≤c5≤0.4, 0≤d≤0.3, 0≤e≤0.3, and 0≤f≤0.3; more preferably 0≤a1≤0.7, 0≤a2≤0.7, 0≤a3≤0.5, 0≤b1≤0.7, 0≤b2≤0.4, 0≤c1≤0.3, 0≤c2≤0.3, 0≤c3≤0.3, 0≤c4≤0.3, 0≤c5≤0.3, 0≤d≤0.2, 0≤e≤0.2, and 0≤f≤0.2; with the proviso that a1+a2+a3+b1+b2+c1+c2+c3+c4+c5+d+e+f≤1.0.

The polymer should preferably have a weight average molecular weight (Mw) in the range of 1,000 to 500,000, and more preferably 3,000 to 100,000, as measured by GPC versus polystyrene standards using tetrahydrofuran (THF) or N,N-dimethylformamide (DMF) solvent. A Mw in the range ensures that the resist film has etch resistance and eliminates the risk of resolution decline by a failure to provide a difference in dissolution rate before and after exposure.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form a ultra-high-molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted by an acetal group which is susceptible to deprotection with acid, typically ethoxyethoxy, prior to polymerization, and the polymerization is followed by deprotection with weak acid and water. Alternatively, the hydroxy group is substituted by an acetyl, formyl or pivaloyl group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (B) may be a single polymer or a blend of two or more polymers which differ in compositional ratio, Mw and/or Mw/Mn. Component (B) may also be a blend of the polymer defined above and a hydrogenated product of ROMP. For the ROMP, reference is made to JP-A 2003-066612.

### (C) Organic Solvent

The resist composition may comprise an organic solvent as component (C). The organic solvent (C) is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate (EL), ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, ethyl lactate, DAA and mixtures thereof because the base polymer (B) is most soluble therein.

The organic solvent (C) is preferably added in an amount of 200 to 5,000 parts by weight, and more preferably 400 to 3,500 parts by weight per 80 parts by weight of the base polymer (B). The organic solvent may be used alone or in admixture.

### (D) Photoacid Generator

The chemically amplified resist composition may comprise (D) a photoacid generator. The PAG is not particularly limited as long as it is capable of generating a strong acid upon exposure to high-energy radiation.

The preferred PAG is a salt having the formula (2) or (3).

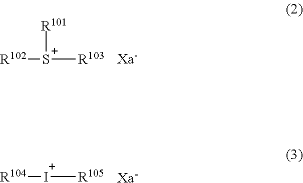

In formula (2), R¹⁰¹ to R¹⁰⁵ are each independently halogen or a C₁-C₂₀ hydrocarbyl group which may contain a heteroatom. Any two of R¹⁰¹, R¹⁰² and R 103 may bond together to form a ring with the sulfur atom to which they are attached. Examples of the hydrocarbyl group are as exemplified above for R¹ to R³ in formula (1).

Non-limiting examples of the cation in the sulfonium salt having formula (2) include those exemplified above for the cation in the sulfonium salt having formula (1), those described in JP-A 2024-003744, paragraphs [0102]-[0125], WO 2024/128017, paragraphs [0044]-[0049], and JP 7491173, paragraphs [0035]-[0046], and those exemplified above for the sulfonium cation having formula (sulfo-1). Non-limiting examples of the cation in the iodonium salt having formula (3) include those described in JP-A 2024-000259, paragraph [0181].

In formulae (2) and (3), Xa⁻ is an anion of strong acid. Examples of the strong acid anion include those having formulae (c1-1) to (c1-5).

Compounds having the formula (4) are also preferred as the PAG (D).

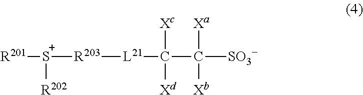

In formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl group represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; and C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl, and combinations thereof. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In formula (4), L²¹ is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (4), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

Of the PAGs having formula (4), those having formula (4′) are preferred.

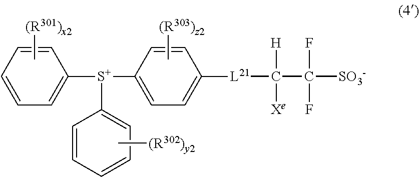

In formula (4′), L²¹ is as defined above. X^{c} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (c1-1-1). The subscripts x2 and y2 are each independently 0, 1, 2, 3, 4 or 5, and z2 is 0, 1, 2, 3 or 4.

Examples of the PAG having formula (4) include those exemplified for the PAG having formula (2) in JP-A 2017-026980.

Of the foregoing PAGs, those having an anion of formula (c1-1-1) or (c1-4) are especially preferred because of reduced acid diffusion and high solubility in solvents. Also those having formula (4′) are especially preferred because of extremely reduced acid diffusion.

When used, the PAG (D) is preferably added in an amount of 0.1 to 40 parts, and more preferably 0.5 to 20 parts by weight per 80 parts by weight of the base polymer (B). As long as the amount of the PAG is in the range, good resolution is achievable and the risk of foreign particles being formed after development or during stripping of resist film is avoided. The PAG may be used alone or in admixture.

### (E) Other Quencher

The resist composition may further comprise (E) a quencher other than component (A).

Onium salts having the formulae (5) and (6) are useful as the other quencher (E).

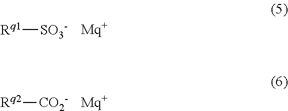

In formula (5), R^{q1} is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In formula (6), R^{q2} is hydrogen or a C₁-C₄₀ hydrocarbyl group which may contain a heteroatom.

Examples of the C₁-C₄₀ hydrocarbyl group R^{q1} include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

Examples of the hydrocarbyl group R^{q2} include those exemplified above for Ral and fluorinated saturated hydrocarbyl groups, for example, fluorinated alkyl groups such as trifluoromethyl and trifluoroethyl, and fluorinated aryl groups such as pentafluorophenyl and 4-trifluoromethylphenyl.

Examples of the anion in the onium salt having formula (5) are shown below, but not limited thereto.

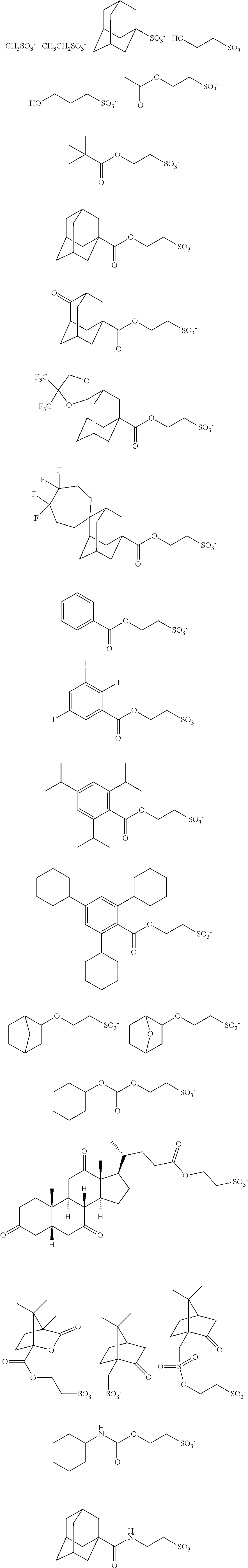

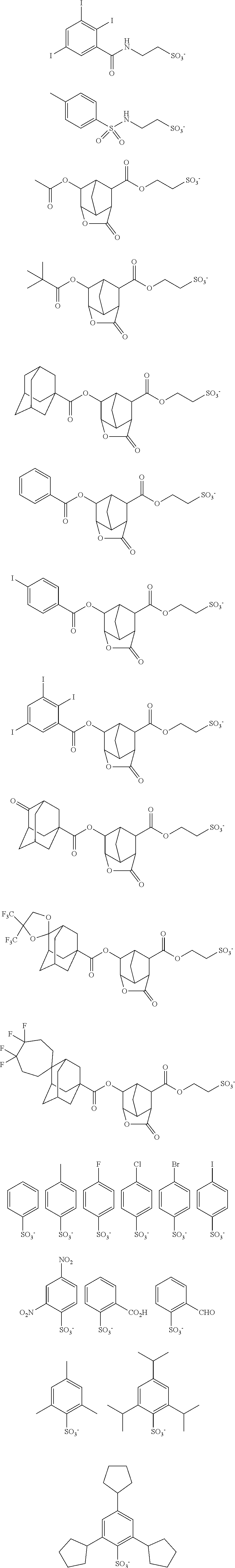

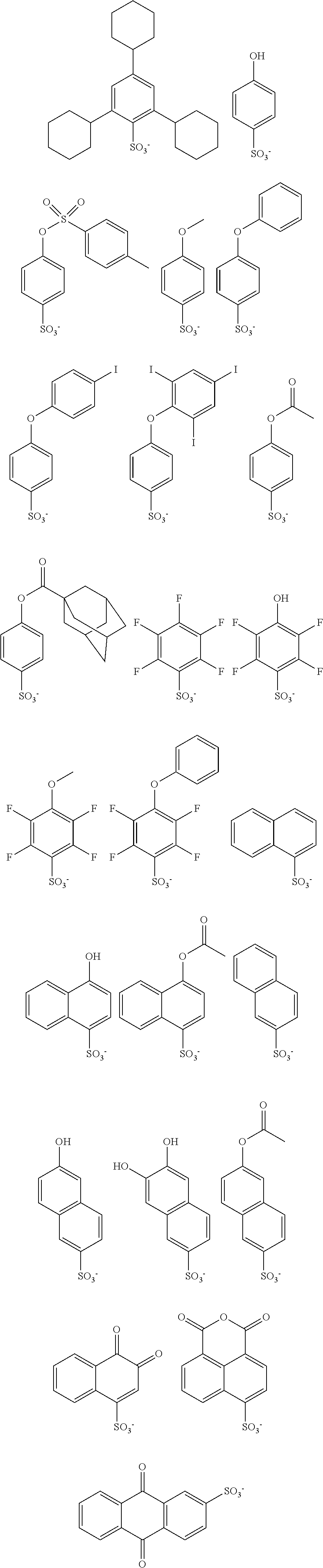

Examples of the anion in the onium salt having formula (6) are shown below, but not limited thereto.

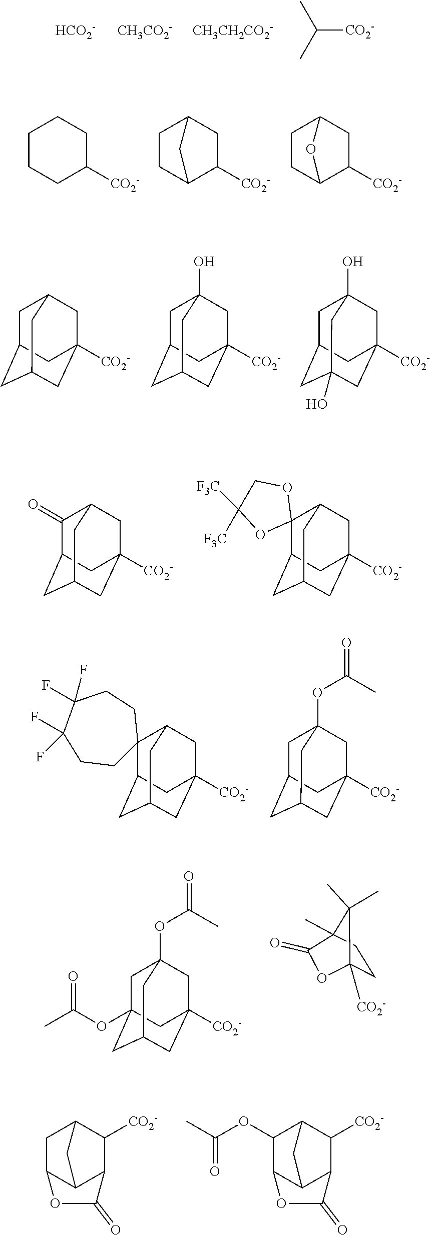

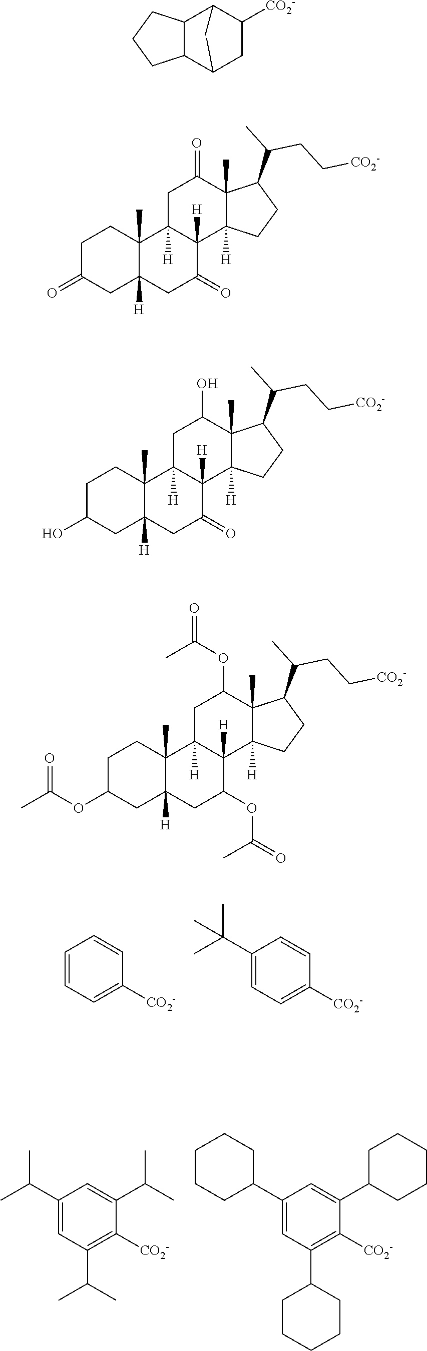

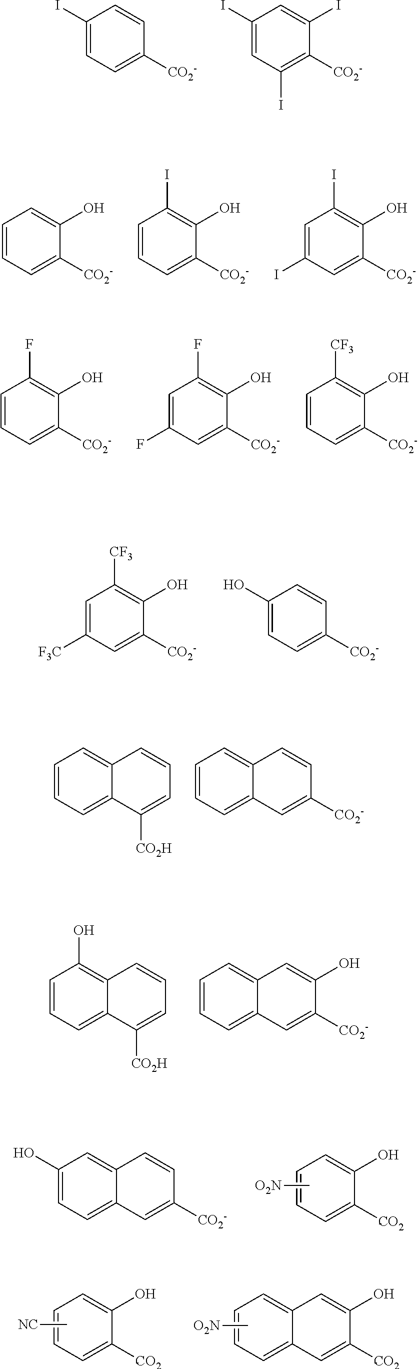

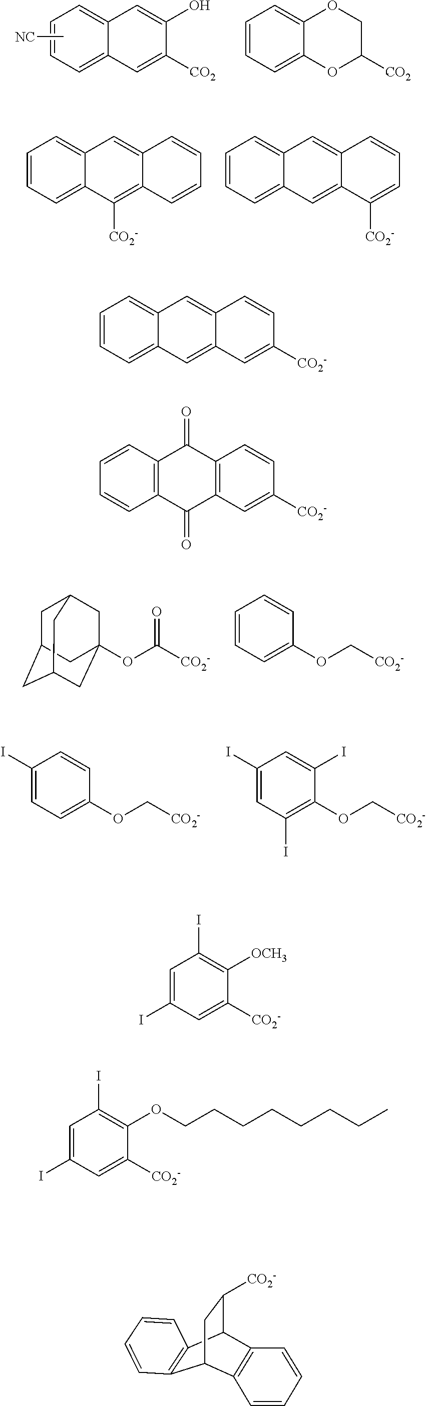

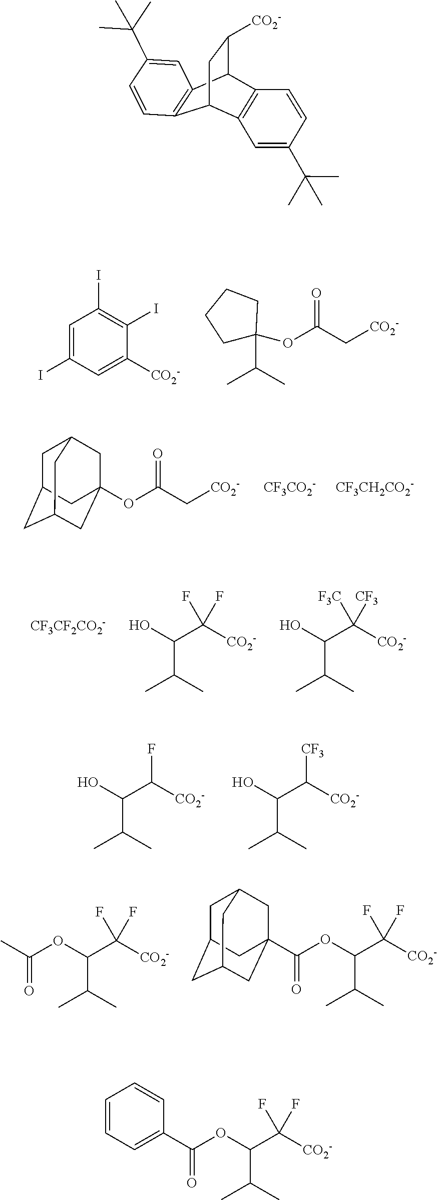

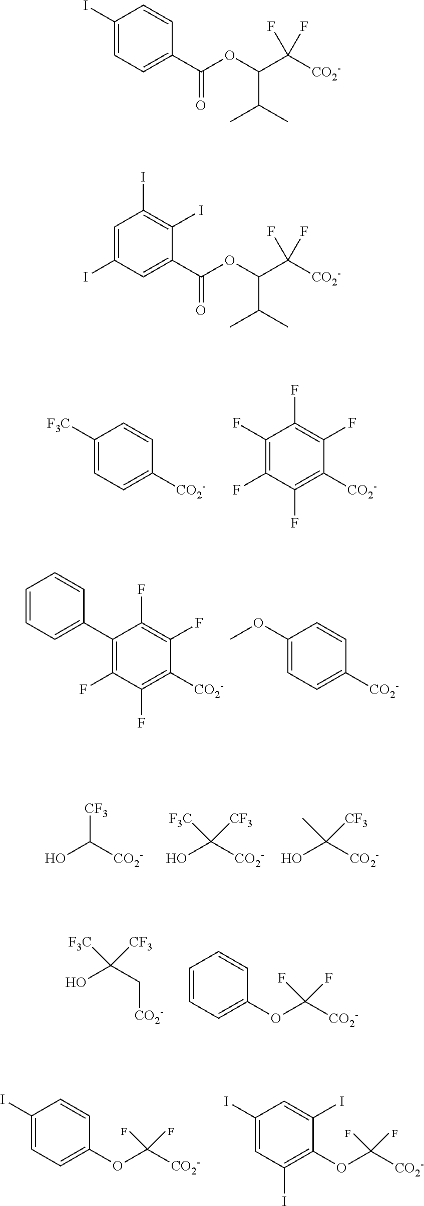

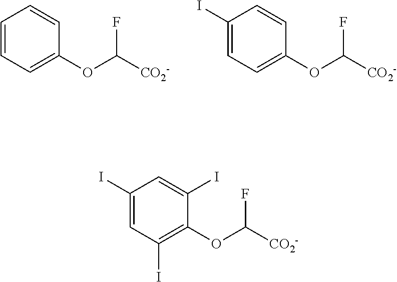

In formulae (5) and (6), Mq⁺ is an onium cation. Suitable onium cations include sulfonium, iodonium, and ammonium cations. Non-limiting examples of the sulfonium cation include those exemplified above for the cation in the sulfonium salt having formula (1), those described in JP-A 2024-003744, paragraphs [0102]-[0125], WO 2024/128017, paragraphs [0044]-[0049], and JP 7491173, paragraphs [0035]-[0046], and those exemplified above for the sulfonium cation having formula (sulfo-1). Non-limiting examples of the iodonium cation include those described in JP-A 2024-000259, paragraph [0181]. Examples of the ammonium cation include those exemplified above for the ammonium cation having formula (am-1).

Examples of the onium salt having formula (5) or (6) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (5) or (6) functions as a quencher in the chemically amplified resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (5) or (6) functions as a quencher when used in combination with an onium salt type PAG having a conjugated base of a strong acid (typically sulfonic acid) as the counter anion. In a system using a mixture of an onium salt capable of generating a strong acid (typically sulfonic acid) and an onium salt capable of generating a weak acid (typically carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the other quencher (E) are onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755.

If a PAG capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the onium salt having formula (5) or (6) is used as the other quencher (E), the amount of the onium salt used is preferably 0.1 to 20 parts by weight, more preferably 0.1 to 10 parts by weight per 80 parts by weight of the base polymer (B). As long as the amount of onium salt type quencher (E) is in the range, a satisfactory resolution is available without a substantial lowering of sensitivity. The onium salt having formula (5) or (6) may be used alone or in admixture.

Nitrogen-containing compounds may also be used as the other quencher (E). Suitable nitrogen-containing compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164] (U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the other quencher (E), the amount of the nitrogen-containing compound used is preferably 0.001 to 12 parts by weight, more preferably 0.01 to 8 parts by weight per 80 parts by weight of the base polymer (B). The nitrogen-containing compound may be used alone or in admixture.

### (F) Surfactant

The resist composition may further include (F) a surfactant. Preferred are a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

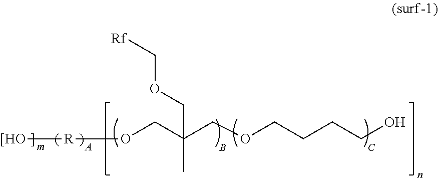

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

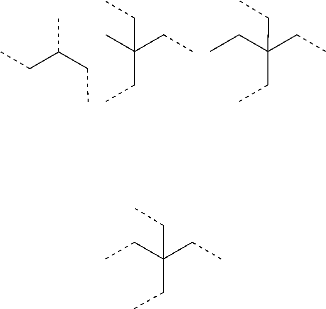

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and Cis 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from repeat units having the formula (7A), repeat units having the formula (7B), repeat units having the formula (7C), repeat units having the formula (7D), and repeat units having the formula (7E), which are also referred to as repeat units 7A to 7E, respectively.

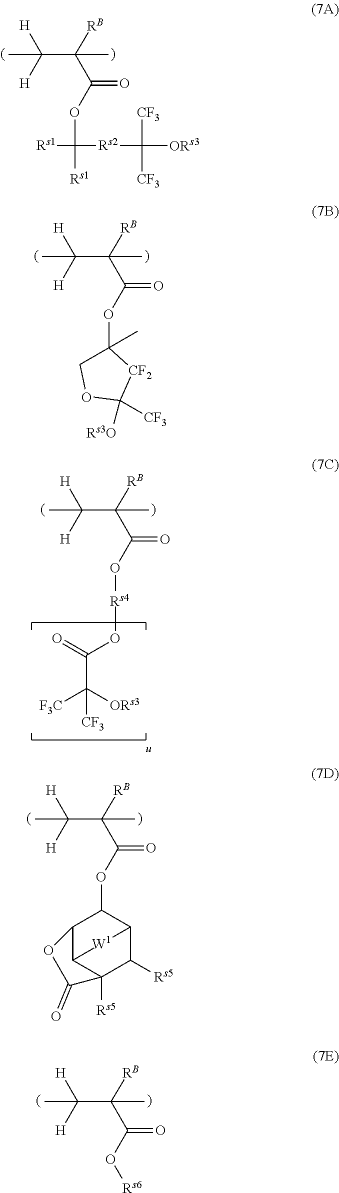

In formulae (7A) to (7E), R^{B} is hydrogen, fluorine, methyl or trifluoromethyl. W¹ is —CH₂—, —CH₂CH₂—, —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is 1, 2 or 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic and is preferably saturated. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group represented by R^{s2} may be straight, branched or cyclic and is preferably saturated. Examples thereof include methylene, ethylene, propylene, butylene and pentylene.

The hydrocarbyl group represented by R^{s3} or R^{s6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic and examples thereof include the foregoing hydrocarbyl or fluorinated hydrocarbyl groups from which “u” number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units 7A to 7E are shown below, but not limited thereto. Herein R^{B} is as defined above.

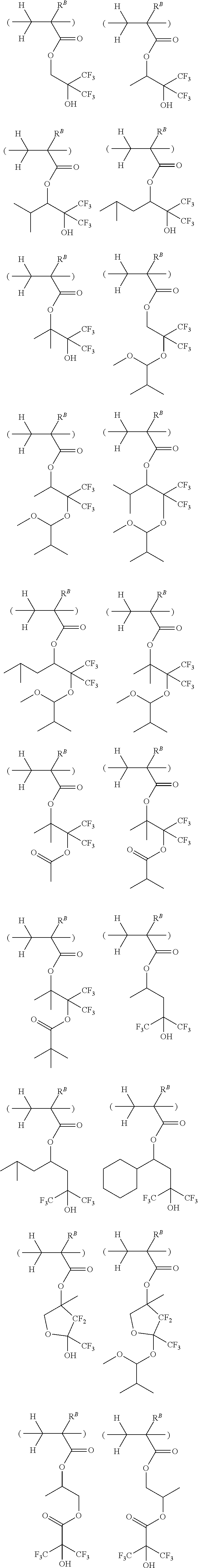

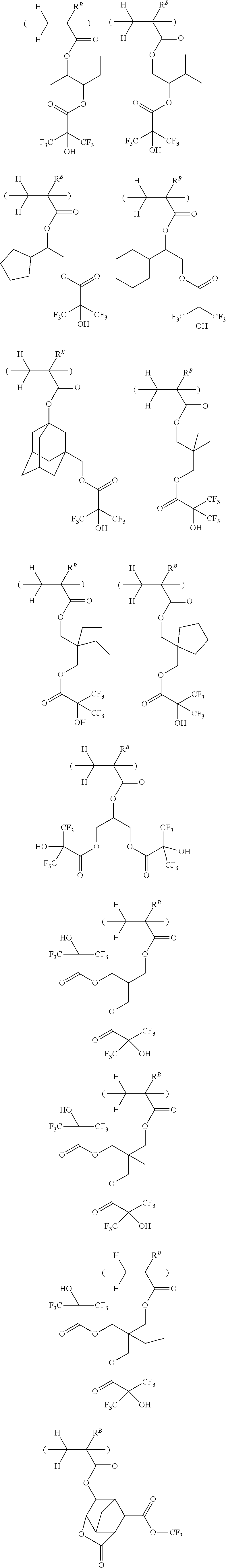

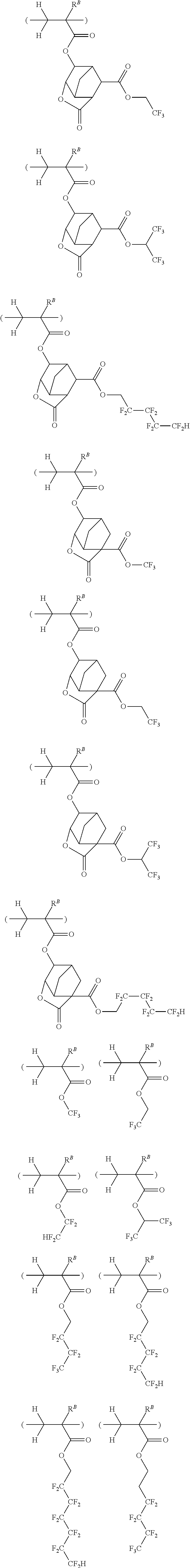

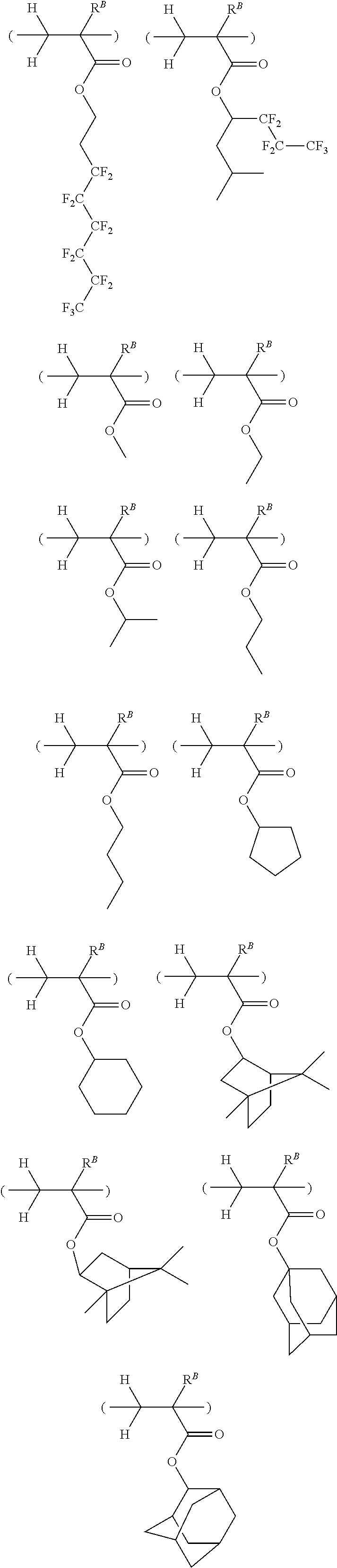

The polymeric surfactant may further contain repeat units other than the repeat units 7A to 7E. Typical other repeat units are those derived from methacrylic acid and α-trifluoromethylacrylic acid derivatives. In the polymeric surfactant, the content of the repeat units 7A to 7E is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units 7A to 7E and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (F), the amount thereof is preferably 0.1 to 50 parts by weight, and more preferably 0.5 to 10 parts by weight per 80 parts by weight of the base polymer (B). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (F) may be used alone or in admixture.

### (G) Other Components

The resist composition may further comprise (G) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (B). An extra amount of the acid amplifier compound can make the acid diffusion control difficult and cause degradations to resolution and pattern profile. With respect to the remaining additives, reference should be made to JP-A 2009-269953 and JP-A 2010-215608.

### [Process]

A further embodiment of the invention is a process of forming a pattern from the resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TIN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Synthesis Examples, Examples and Comparative Examples are given below by way of illustration and not by way of limitation. The abbreviation “pbw” is parts by weight. Analysis is made by time-of-flight mass spectrometry using the instrument, MALDI TOF-MS: S3000 by JEOL Ltd.

### [1] Synthesis of Sulfonium Salts

### Example 1-1

### Synthesis of Sulfonium Salt PDQ-1

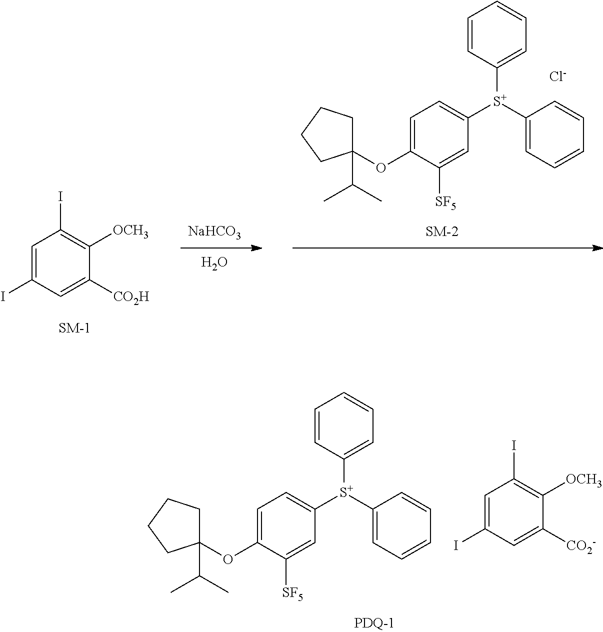

In a reactor under nitrogen atmosphere, 4.0 g of reactant SM-1, 1.0 g of sodium hydrogencarbonate and 20 g of water were mixed and stirred at an internal temperature of 45° C. for 1 hour. The reaction solution was cooled to room temperature. Thereafter, 5.5 g of reactant SM-2 and 30 g of methyl isobutyl ketone were added to the solution, which was stirred at room temperature for 30 minutes. At the end of stirring, the organic layer was taken out, washed with water, and concentrated under reduced pressure. The concentrate was purified by silica gel chromatography, obtaining the target sulfonium salt PDQ-1 as oily matter (amount 7.7 g, yield 84%).

PDQ-1 was analyzed by TOF-MS, with the data shown below.

### MALDI TOF-MS:

positive M⁺515 (corresponding to C₂₆H₂₈F₅OS₂)
negative M⁻ 403 (corresponding to C₈H₅I₂O₃⁻)

### Examples 1-2 to 1-7

### Synthesis of Sulfonium Salts PDQ-2 to PDQ-7

Sulfonium salts PDQ-2 to PDQ-7 shown below were synthesized using corresponding reactants and well-known organic synthesis reactions.

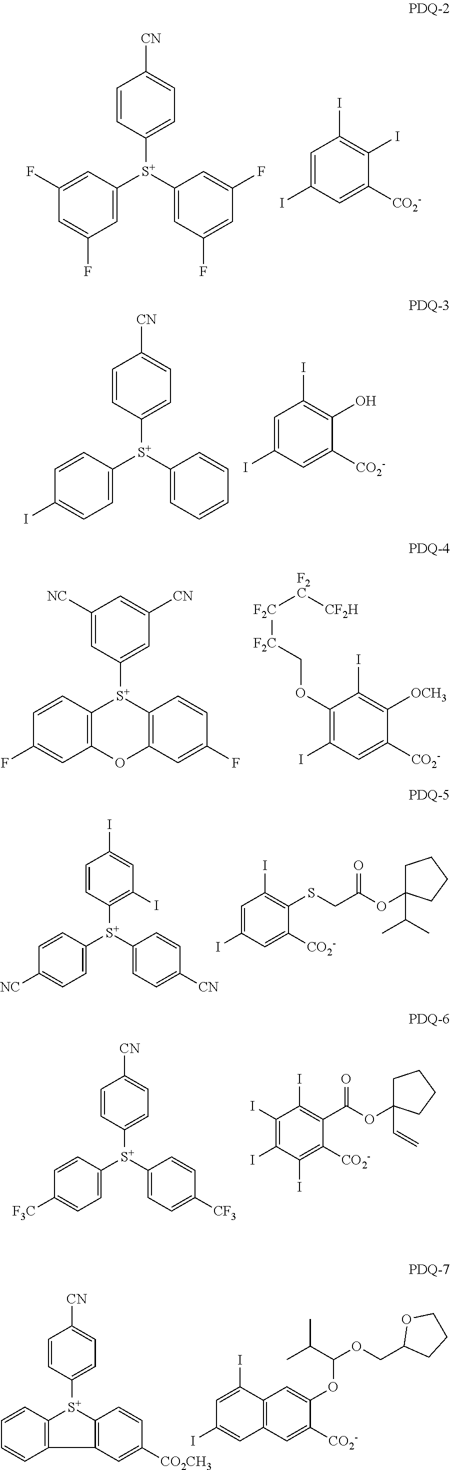

### [2] Synthesis of Base Polymers

### Synthesis Example

### Synthesis of Base Polymers P-1 to P-5

Base polymers P-1 to P-5 were synthesized by combining monomers, performing copolymerization reaction in MEK solvent, pouring the reaction solution to hexane for precipitation, washing the solid precipitate with hexane, isolation and drying. The polymer was analyzed for composition by ¹H-NMR spectroscopy and for Mw and Mw/Mn by GPC versus polystyrene standards using DMF solvent.

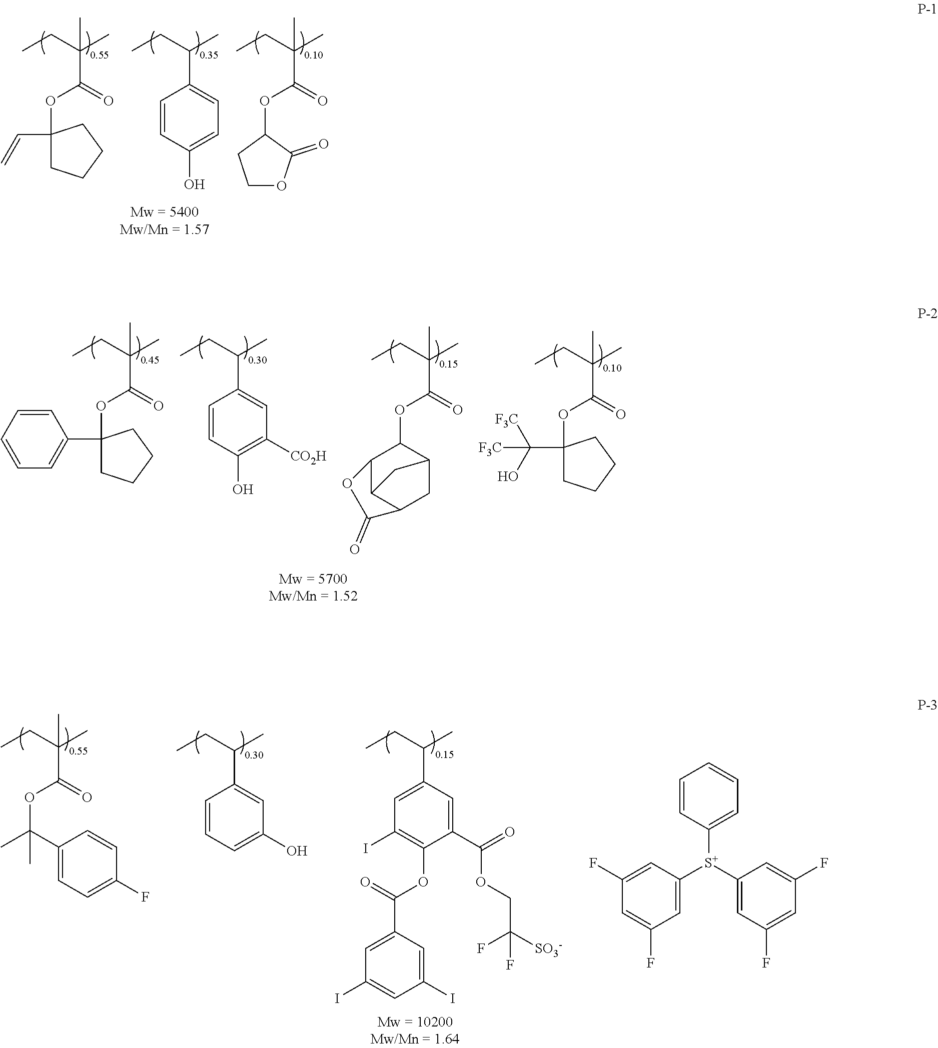

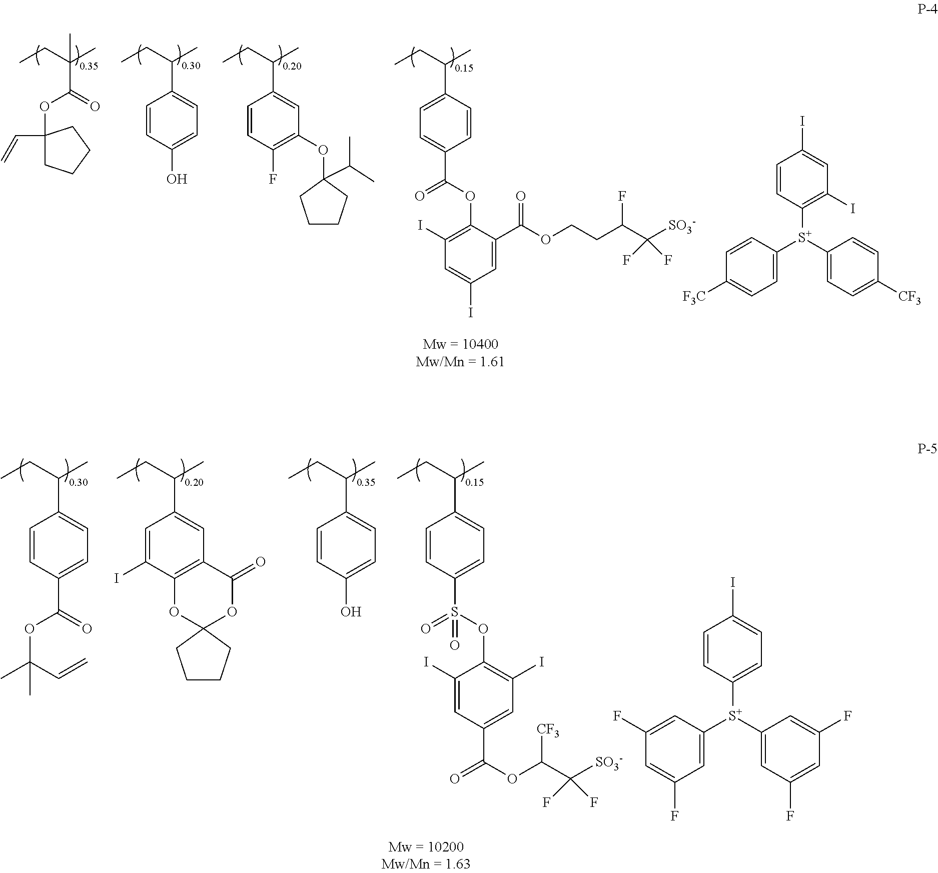

### [3] Preparation of Chemically Amplified Resist Compositions

### Examples 2-1 to 2-30 and Comparative Examples 1-1 to 1-20

Chemically amplified resist compositions (R-1 to R-30, CR-1 to CR-20) in solution 5 form were prepared by dissolving a quencher (PDQ-1 to PDQ-7) or comparative quencher (PDQ-A to PDQ-D), photoacid generator (PAG-X, PAG-Y and PAG-Z), base polymer (P-1 to P-5), and other quencher (AQ-1 and AQ-2) in a solvent containing 0.01 wt % of surfactant A in accordance with the formulation shown in Tables 1 and 2, and filtering through a Teflon® filter with a pore size of 0.2 μm.

TABLE 1

| | | | Base | | Photoacid | Other | | | |
|---|---|---|---|---|---|---|---|---|---|
| | | Resist | polymer | Quencher | generator | quencher | Solvent 1 | Solvent 2 | Solvent 3 |
| | | composition | (pbw) | (pbw) | (pbw) | (pbw) | (pbw) | (pbw) | (pbw) |
| Example | 2-1 | R-1 | P-1 (80) | PDQ-1 (5) | PAG-X (15) | — | PGMEA (2250) | EL (2800) | DAA (550) |
| | 2-2 | R-2 | P-1 (80) | PDQ-2 (5) | PAG-X (15) | — | PGMEA (2250) | EL (2800) | DAA (550) |
| | 2-3 | R-3 | P-1 (80) | PDQ-3 (5) | PAG-X (15) | — | PGMEA (2250) | EL (2800) | DAA (550) |
| | 2-4 | R-4 | P-1 (80) | PDQ-4 (5) | PAG-X (15) | — | PGMEA (2250) | EL (2800) | DAA (550) |
| | 2-5 | R-5 | P-1 (80) | PDQ-5 (5) | PAG-X (15) | — | PGMEA (2250) | EL (2800) | DAA (550) |
| | 2-6 | R-6 | P-1 (80) | PDQ-6 (5) | PAG-X (15) | — | PGMEA (2250) | EL (2800) | DAA (550) |
| | 2-7 | R-7 | P-1 (80) | PDQ-7 (5) | PAG-X (15) | — | PGMEA (2250) | EL (2800) | DAA (550) |
| | 2-8 | R-8 | P-1 (80) | PDQ-1 (5) | PAG-Y (15) | — | PGMEA (2250) | EL (2800) | DAA (550) |
| | 2-9 | R-9 | P-1 (80) | PDQ-1 (2.5) | PAG-X (15) | AQ-1 (2.5) | PGMEA (2250) | EL (2800) | DAA (550) |
| | 2-10 | R-10 | P-1 (80) | PDQ-2 (3) | PAG-Y (15) | AQ-2 (2) | PGMEA (2250) | EL (2800) | DAA (550) |
| | 2-11 | R-11 | P-2 (80) | PDQ-1 (5) | PAG-X (14) | — | PGMEA (2250) | EL (2800) | DAA (550) |
| | 2-12 | R-12 | P-2 (80) | PDQ-2 (5) | PAG-X (15) | — | PGMEA (2250) | EL (2800) | DAA (550) |
| | 2-13 | R-13 | P-2 (80) | PDQ-4 (5) | PAG-Y (14) | — | PGMEA (2250) | EL (2800) | DAA (550) |
| | 2-14 | R-14 | P-2 (80) | PDQ-6 (5) | PAG-Y (15) | — | PGMEA (2250) | EL (2800) | DAA (550) |
| | 2-15 | R-15 | P-2 (80) | PDQ-1 (4) | PAG-X (10) | AQ-1 (2) | PGMEA (2250) | EL (2800) | DAA (550) |
| | | | | | PAG-Z (5) | | | | |
| | 2-16 | R-16 | P-3 (80) | PDQ-1 (5) | — | — | PGMEA (2250) | EL (2800) | DAA (550) |
| | 2-17 | R-17 | P-3 (80) | PDQ-2 (5) | — | — | PGMEA (2250) | EL (2800) | DAA (550) |
| | 2-18 | R-18 | P-3 (80) | PDQ-3 (5) | — | — | PGMEA (2250) | EL (2800) | DAA (550) |
| | 2-19 | R-19 | P-3 (80) | PDQ-4 (5) | PAG-X (5) | — | PGMEA (2250) | EL (2800) | DAA (550) |
| | 2-20 | R-20 | P-3 (80) | PDQ-2 (3) | — | AQ-1 (2) | PGMEA (2250) | EL (2800) | DAA (550) |
| | 2-21 | R-21 | P-4 (80) | PDQ-1 (5) | — | — | PGMEA (2250) | EL (2800) | DAA (550) |
| | 2-22 | R-22 | P-4 (80) | PDQ-2 (5) | — | — | PGMEA (2250) | EL (2800) | DAA (550) |
| | 2-23 | R-23 | P-4 (80) | PDQ-5 (5) | — | — | PGMEA (2250) | EL (2800) | DAA (550) |
| | 2-24 | R-24 | P-4 (80) | PDQ-6 (6) | PAG-Y (4) | — | PGMEA (2250) | EL (2800) | DAA (550) |
| | | | | | PAG-Z (2) | | | | |
| | 2-25 | R-25 | P-4 (80) | PDQ-2 (5) | — | — | PGMEA (2250) | EL (2800) | DAA (550) |
| | 2-26 | R-26 | P-5 (80) | PDQ-1 (5) | — | — | PGMEA (2250) | EL (2800) | DAA (550) |
| | 2-27 | R-27 | P-5 (80) | PDQ-2 (5) | — | — | PGMEA (2250) | EL (2800) | DAA (550) |
| | 2-28 | R-28 | P-5 (80) | PDQ-7 (3) | PAG-Y (5) | AQ-2 (2) | PGMEA (2250) | EL (2800) | DAA (550) |
| | 2-29 | R-29 | P-5 (80) | PDQ-3 (5) | — | — | PGMEA (2250) | EL (2800) | DAA (550) |
| | 2-30 | R-30 | P-5 (80) | PDQ-2 (2.5) | — | AQ-1 (2.5) | PGMEA (2250) | EL (2800) | DAA (550) |

TABLE 2

| | | | Base | Comparative | Photoacid | Other | | | |
|---|---|---|---|---|---|---|---|---|---|
| | | Resist | polymer | quencher | generator | quencher | Solvent 1 | Solvent 2 | Solvent 3 |
| | | composition | (pbw) | (pbw) | (pbw) | (pbw) | (pbw) | (pbw) | (pbw) |
| Comparative | 1-1 | CR-1 | P-1 (80) | PDQ-A (5) | PAG-X (15) | — | PGMEA (2250) | EL (2800) | DAA (550) |
| Example | 1-2 | CR-2 | P-1 (80) | PDQ-B (5) | PAG-X (15) | — | PGMEA (2250) | EL (2800) | DAA (550) |
| | 1-3 | CR-3 | P-1 (80) | PDQ-C (5) | PAG-X (15) | — | PGMEA (2250) | EL (2800) | DAA (550) |
| | 1-4 | CR-4 | P-1 (80) | PDQ-D (5) | PAG-X (15) | — | PGMEA (2250) | EL (2800) | DAA (550) |
| | 1-5 | CR-5 | P-1 (80) | PDQ-A (5) | PAG-Y (15) | — | PGMEA (2250) | EL (2800) | DAA (550) |
| | 1-6 | CR-6 | P-1 (80) | PDQ-B (5) | PAG-Y (15) | — | PGMEA (2250) | EL (2800) | DAA (550) |
| | 1-7 | CR-7 | P-1 (80) | PDQ-C (2.5) | PAG-X (15) | AQ-1 (2.5) | PGMEA (2250) | EL (2800) | DAA (550) |
| | 1-8 | CR-8 | P-1 (80) | PDQ-D (3) | PAG-Y (15) | AQ-2 (2) | PGMEA (2250) | EL (2800) | DAA (550) |
| | 1-9 | CR-9 | P-2 (80) | PDQ-A (5) | PAG-X (15) | — | PGMEA (2250) | EL (2800) | DAA (550) |
| | 1-10 | CR-10 | P-2 (80) | PDQ-B (5) | PAG-X (15) | — | PGMEA (2250) | EL (2800) | DAA (550) |
| | 1-11 | CR-11 | P-2 (80) | PDQ-C (4) | PAG-X (10) | AQ-1 (2) | PGMEA (2250) | EL (2800) | DAA (550) |
| | | | | | PAG-Z (5) | | | | |
| | 1-12 | CR-12 | P-3 (80) | PDQ-A (5) | — | — | PGMEA (2250) | EL (2800) | DAA (550) |
| | 1-13 | CR-13 | P-3 (80) | PDQ-C (5) | — | — | PGMEA (2250) | EL (2800) | DAA (550) |
| | 1-14 | CR-14 | P-3 (80) | PDQ-D (3) | — | AQ-1 (2) | PGMEA (2250) | EL (2800) | DAA (550) |
| | 1-15 | CR-15 | P-4 (80) | PDQ-A (5) | — | — | PGMEA (2250) | EL (2800) | DAA (550) |
| | 1-16 | CR-16 | P-4 (80) | PDQ-D (5) | — | — | PGMEA (2250) | EL (2800) | DAA (550) |
| | 1-17 | CR-17 | P-4 (80) | PDQ-C (6) | PAG-Y (4) | — | PGMEA (2250) | EL (2800) | DAA (550) |
| | | | | | PAG-Z (2) | | | | |
| | 1-18 | CR-18 | P-5 (80) | PDQ-A (5) | — | — | PGMEA (2250) | EL (2800) | DAA (550) |
| | 1-19 | CR-19 | P-5 (80) | PDQ-B (3) | PAG-Y (5) | AQ-2 (2) | PGMEA (2250) | EL (2800) | DAA (550) |
| | 1-20 | CR-20 | P-5 (80) | PDQ-C (5) | — | — | PGMEA (2250) | EL (2800) | DAA (550) |

The solvents, photoacid generators PAG-X, PAG-Y and PAG-Z, comparative quenchers PDQ-A to PDQ-D, other quenchers AQ-1 and AQ-2, and surfactant A in Tables 1 and 2 are identified below.

### Solvent:

PGMEA (propylene glycol monomethyl ether acetate)
EL (ethyl lactate)
DAA (diacetone alcohol)

### Photoacid Generators: PAG-X, PAG-Y and PAG-Z

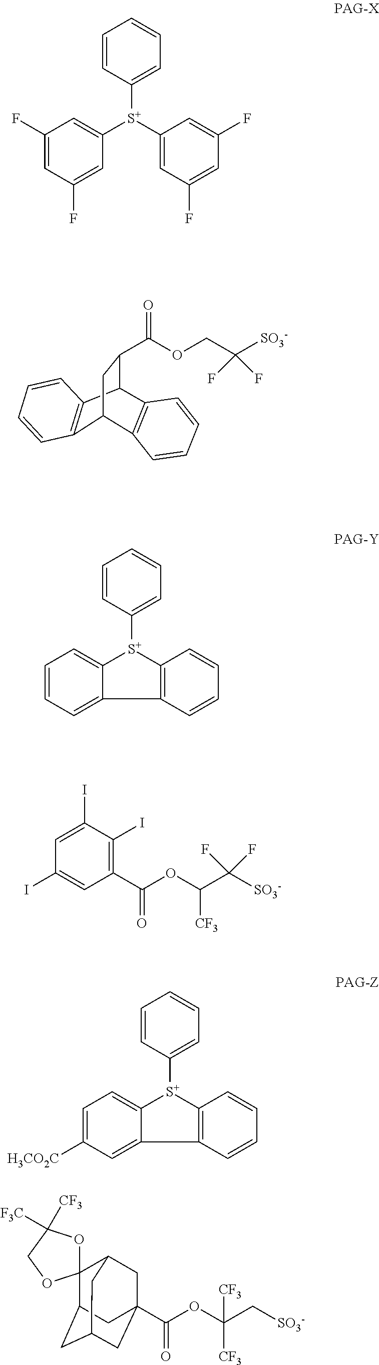

### Comparative Quenchers: PDQ-A to PDQ-D

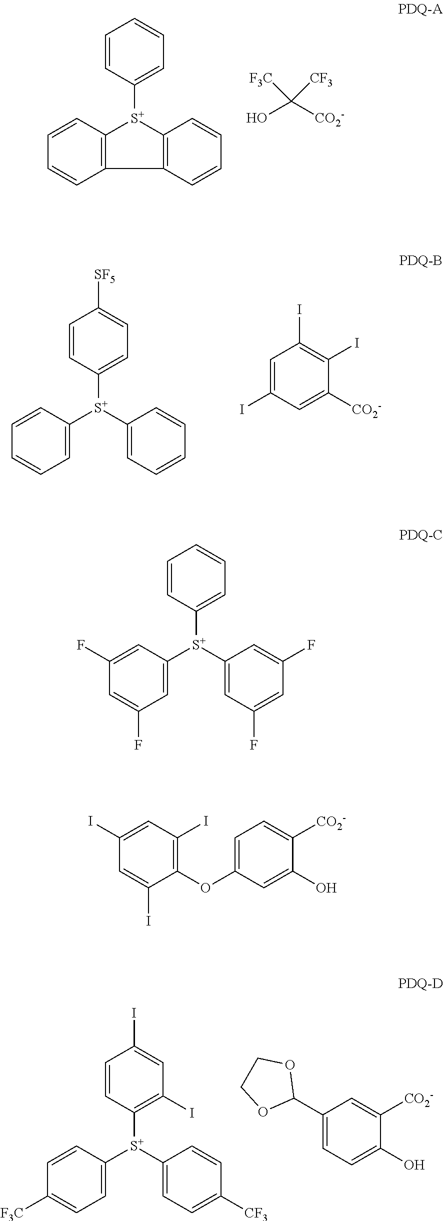

### Other Quenchers: AQ-1 and AQ-2

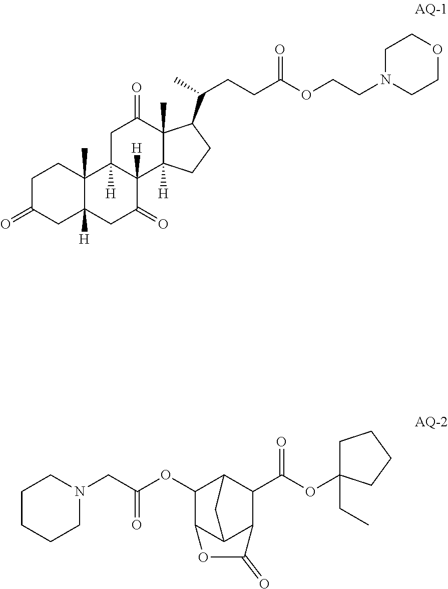

### Surfactant A:

3-methyl-3-(2,2,2-trifluoroethoxymethyl)oxetane/tetrahydrofuran/

2,2-dimethyl-1,3-propane diol copolymer (Omnova Solutions, Inc.)

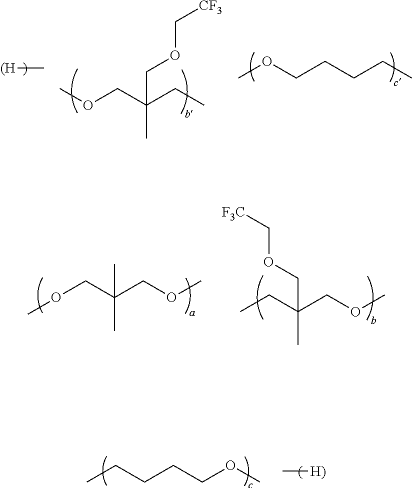

a:(b+b′):(c+c′)=1:4-7:0.01-1 (molar ratio)

Mw=1,500

### [4] EUV Lithography Test 1

### Examples 3-1 to 3-30 and Comparative Examples 2-1 to 2-20

Each of the chemically amplified resist compositions (R-1 to R-30, CR-1 to CR-20) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, ~ 0.9/0.6, dipole illumination), the resist film was exposed to EUV through a mask bearing a line-and-space (LS) pattern having a size of 18 nm and a pitch of 36 nm (on-wafer size) while varying the dose and focus (dose pitch: 1 mJ/cm², focus pitch: 0.020 μm). The resist film was baked (PEB) on a hotplate at the temperature shown in Tables 3 and 4 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, DOF and collapse limit were evaluated by the following methods. The LS pattern was also evaluated for development defectiveness. The results are shown in Tables 3 and 4.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation:

$$EL ( \% ) = ( \vert {E}_{1} - {E}_{2} \vert / Eop ) \times 100$$

wherein E₁ is an optimum exposure dose which provides a LS pattern with a line width of 16.2 nm and a pitch of 36 nm, E2 is an optimum exposure dose which provides a LS pattern with a line width of 19.8 nm and a pitch of 36 nm, and Eop is an optimum exposure dose which provides a LS pattern with a line width of 18 nm and a pitch of 36 nm. A greater value indicates better performance.

### [Evaluation of LWR]

For the LS pattern formed by exposure at the optimum dose Eop, the line width was measured at 10 longitudinally spaced apart points, from which a 3-fold value (3σ) of the standard deviation (σ) was determined and reported as LWR. A smaller value of 3σ indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

### [Evaluation of Development Defects]

The LS pattern of line width 18 nm and pitch 36 nm printed at the optimum dose was counted for defects using a defect inspection system (trade name KLA 2360 by KLA-Tencor Corp.). With a pixel size of 0.16 μm and a threshold of 20 set, the number of defects extracted from the difference obtained by overlapping a comparative image and a pixel unit was detected, from which the count of defects per unit area (count/cm²) was computed. On reviewing defects, development defects were classified and extracted from all the defects. The number of development defects per unit area (count/cm²) was computed. A sample is rated “A” for a count of less than 0.5, “B” for a count of 0.5 to less than 1.0, “C” for a count of 1.0 to less than 5.0, and “D” for a count of 5.0 or more. A smaller count indicates better performance.

TABLE 3

| | | | PEB | | | | | Collapse | |
|---|---|---|---|---|---|---|---|---|---|
| | | Resist | temp. | Eop | EL | LWR | DOF | limit | Development |
| | | composition | (° C.) | (mJ/cm²) | (%) | (nm) | (nm) | (nm) | defects |
| Example | 3-1 | R-1 | 100 | 31 | 19 | 2.2 | 110 | 11.1 | A |
| | 3-2 | R-2 | 100 | 32 | 18 | 2.2 | 120 | 11.0 | A |
| | 3-3 | R-3 | 100 | 31 | 18 | 2.1 | 110 | 10.8 | A |
| | 3-4 | R-4 | 95 | 33 | 17 | 2.2 | 100 | 11.0 | A |
| | 3-5 | R-5 | 100 | 32 | 18 | 2.4 | 110 | 11.2 | A |
| | 3-6 | R-6 | 100 | 31 | 17 | 2.2 | 120 | 10.8 | A |
| | 3-7 | R-7 | 105 | 32 | 18 | 2.3 | 120 | 11.2 | A |
| | 3-8 | R-8 | 100 | 32 | 19 | 2.1 | 120 | 10.9 | A |
| | 3-9 | R-9 | 100 | 31 | 18 | 2.2 | 110 | 11.1 | A |
| | 3-10 | R-10 | 100 | 31 | 17 | 2.3 | 120 | 11.0 | A |
| | 3-11 | R-11 | 105 | 31 | 19 | 2.2 | 110 | 10.7 | A |
| | 3-12 | R-12 | 100 | 32 | 18 | 2.1 | 100 | 11.4 | A |
| | 3-13 | R-13 | 95 | 31 | 16 | 2.2 | 110 | 10.9 | A |
| | 3-14 | R-14 | 100 | 32 | 17 | 2.2 | 120 | 10.8 | A |
| | 3-15 | R-15 | 100 | 32 | 18 | 2.3 | 100 | 11.3 | A |
| | 3-16 | R-16 | 100 | 32 | 19 | 2.1 | 110 | 11.0 | A |
| | 3-17 | R-17 | 100 | 33 | 18 | 2.4 | 120 | 11.1 | A |
| | 3-18 | R-18 | 100 | 34 | 17 | 2.3 | 100 | 11.2 | A |
| | 3-19 | R-19 | 100 | 31 | 18 | 2.2 | 120 | 11.2 | A |
| | 3-20 | R-20 | 100 | 32 | 19 | 2.1 | 110 | 11.2 | A |
| | 3-21 | R-21 | 100 | 33 | 17 | 2.2 | 120 | 11.1 | A |
| | 3-22 | R-22 | 105 | 31 | 18 | 2.4 | 110 | 10.8 | A |
| | 3-23 | R-23 | 100 | 32 | 19 | 2.1 | 120 | 11.2 | A |
| | 3-24 | R-24 | 100 | 32 | 17 | 2.3 | 120 | 11.1 | A |
| | 3-25 | R-25 | 105 | 33 | 17 | 2.2 | 110 | 11.4 | A |
| | 3-26 | R-26 | 100 | 31 | 17 | 2.4 | 100 | 10.9 | A |
| | 3-27 | R-27 | 95 | 31 | 19 | 2.2 | 120 | 10.7 | A |
| | 3-28 | R-28 | 105 | 32 | 18 | 2.2 | 110 | 11.0 | A |
| | 3-29 | R-29 | 100 | 33 | 17 | 2.1 | 110 | 11.2 | A |
| | 3-30 | R-30 | 95 | 32 | 16 | 2.2 | 120 | 11.1 | A |

TABLE 4

| | | | PEB | | | | | Collapse | |
|---|---|---|---|---|---|---|---|---|---|
| | | Resist | temp. | Eop | EL | LWR | DOF | limit | Development |
| | | composition | (° C.) | (mJ/cm²) | (%) | (nm) | (nm) | (nm) | defects |
| Comparative | 2-1 | CR-1 | 100 | 36 | 12 | 3.0 | 70 | 14.2 | B |
| Example | 2-2 | CR-2 | 100 | 35 | 13 | 2.8 | 90 | 12.4 | B |
| | 2-3 | CR-3 | 105 | 37 | 13 | 3.1 | 70 | 12.4 | C |
| | 2-4 | CR-4 | 100 | 36 | 15 | 3.1 | 80 | 12.4 | B |
| | 2-5 | CR-5 | 100 | 35 | 12 | 2.8 | 80 | 13.1 | B |
| | 2-6 | CR-6 | 100 | 41 | 13 | 2.7 | 70 | 13.2 | C |
| | 2-7 | CR-7 | 100 | 40 | 13 | 3.1 | 70 | 12.6 | B |
| | 2-8 | CR-8 | 100 | 38 | 14 | 3.0 | 80 | 12.7 | C |
| | 2-9 | CR-9 | 105 | 36 | 13 | 3.2 | 90 | 13.1 | B |
| | 2-10 | CR-10 | 100 | 35 | 12 | 2.8 | 90 | 13.2 | C |
| | 2-11 | CR-11 | 100 | 36 | 15 | 3.1 | 70 | 12.6 | B |
| | 2-12 | CR-12 | 100 | 37 | 13 | 3.1 | 90 | 13.1 | B |
| | 2-13 | CR-13 | 100 | 38 | 13 | 2.7 | 100 | 12.8 | C |
| | 2-14 | CR-14 | 95 | 41 | 15 | 3.0 | 90 | 12.8 | B |
| | 2-15 | CR-15 | 105 | 39 | 13 | 3.1 | 100 | 13.1 | C |
| | 2-16 | CR-16 | 95 | 36 | 14 | 3.3 | 70 | 13.1 | B |
| | 2-17 | CR-17 | 100 | 38 | 14 | 2.8 | 90 | 12.8 | C |
| | 2-18 | CR-18 | 100 | 37 | 15 | 2.7 | 80 | 12.7 | B |
| | 2-19 | CR-19 | 100 | 40 | 13 | 2.8 | 70 | 12.6 | C |
| | 2-20 | CR-20 | 100 | 39 | 13 | 3.1 | 90 | 13.1 | B |

It is demonstrated in Tables 3 and 4 that chemically amplified resist compositions 5 comprising quenchers within the scope of the invention exhibit a high sensitivity and improved values of EL, LWR and DOF. Small values of collapse limit attest that in forming a small-size pattern, the pattern is resistant to collapse. The restraint of development defects is also confirmed. The resist compositions are useful in the EUV lithography process.

### [5] EUV Lithography Test 2

### Examples 4-1 to 4-30 and Comparative Examples 3-1 to 3-20

Each of the chemically amplified resist compositions (R-1 to R-30, CR-1 to CR-20) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 5 and 6 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as CDU. The results are shown in Tables 5 and 6.

TABLE 5

| | | Resist | PEB temp. | Eop | CDU |
|---|---|---|---|---|---|
| | | composition | (° C.) | (mJ/cm²) | (nm) |
| Example | 4-1 | R-1 | 90 | 21 | 2.2 |
| | 4-2 | R-2 | 90 | 22 | 2.3 |
| | 4-3 | R-3 | 95 | 22 | 2.2 |
| | 4-4 | R-4 | 90 | 23 | 2.3 |
| | 4-5 | R-5 | 90 | 22 | 2.5 |
| | 4-6 | R-6 | 90 | 23 | 2.3 |
| | 4-7 | R-7 | 95 | 21 | 2.4 |
| | 4-8 | R-8 | 95 | 24 | 2.2 |
| | 4-9 | R-9 | 90 | 22 | 2.4 |
| | 4-10 | R-10 | 95 | 21 | 2.2 |
| | 4-11 | R-11 | 95 | 22 | 2.3 |
| | 4-12 | R-12 | 90 | 24 | 2.3 |
| | 4-13 | R-13 | 90 | 23 | 2.2 |
| | 4-14 | R-14 | 95 | 22 | 2.3 |
| | 4-15 | R-15 | 95 | 24 | 2.5 |
| | 4-16 | R-16 | 85 | 22 | 2.2 |
| | 4-17 | R-17 | 95 | 24 | 2.3 |
| | 4-18 | R-18 | 90 | 23 | 2.3 |
| | 4-19 | R-19 | 95 | 21 | 2.3 |
| | 4-20 | R-20 | 90 | 22 | 2.4 |
| | 4-21 | R-21 | 90 | 24 | 2.2 |
| | 4-22 | R-22 | 90 | 22 | 2.4 |
| | 4-23 | R-23 | 95 | 24 | 2.3 |
| | 4-24 | R-24 | 90 | 23 | 2.4 |
| | 4-25 | R-25 | 90 | 24 | 2.2 |
| | 4-26 | R-26 | 85 | 22 | 2.3 |
| | 4-27 | R-27 | 95 | 23 | 2.4 |
| | 4-28 | R-28 | 90 | 22 | 2.5 |
| | 4-29 | R-29 | 90 | 22 | 2.3 |
| | 4-30 | R-30 | 95 | 22 | 2.3 |

TABLE 6

| | | Resist | PEB temp. | Eop | CDU |
|---|---|---|---|---|---|
| | | composition | (° C.) | (mJ/cm²) | (nm) |
| Comparative | 3-1 | CR-1 | 90 | 28 | 3.1 |
| Example | 3-2 | CR-2 | 90 | 29 | 2.9 |
| | 3-3 | CR-3 | 95 | 29 | 2.9 |
| | 3-4 | CR-4 | 90 | 29 | 2.8 |
| | 3-5 | CR-5 | 95 | 31 | 3.1 |
| | 3-6 | CR-6 | 85 | 27 | 3.2 |
| | 3-7 | CR-7 | 90 | 28 | 2.8 |
| | 3-8 | CR-8 | 90 | 28 | 2.8 |
| | 3-9 | CR-9 | 90 | 29 | 2.9 |
| | 3-10 | CR-10 | 95 | 29 | 2.7 |
| | 3-11 | CR-11 | 90 | 27 | 3.1 |
| | 3-12 | CR-12 | 90 | 28 | 2.8 |
| | 3-13 | CR-13 | 95 | 29 | 2.9 |
| | 3-14 | CR-14 | 90 | 27 | 3.0 |
| | 3-15 | CR-15 | 90 | 29 | 2.9 |
| | 3-16 | CR-16 | 90 | 28 | 3.0 |
| | 3-17 | CR-17 | 95 | 28 | 3.2 |
| | 3-18 | CR-18 | 90 | 28 | 2.9 |
| | 3-19 | CR-19 | 85 | 27 | 2.7 |
| | 3-20 | CR-20 | 90 | 29 | 2.9 |

It is demonstrated in Tables 5 and 6 that chemically amplified resist compositions within the scope of the invention exhibit a high sensitivity and satisfactory CDU.

Japanese Patent Application No. 2025-010482 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt having the formula (1): 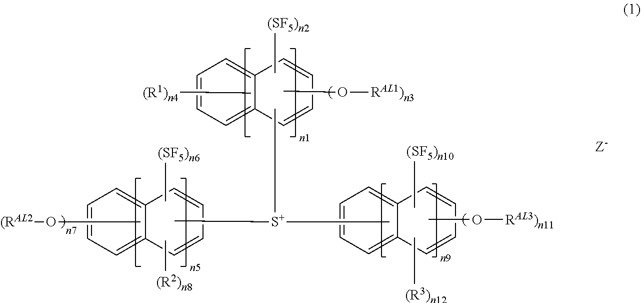
wherein n1 is 0 or 1, n2 is 0, 1 or 2, n3 is 0, 1 or 2, n4 is 0, 1 or 2, meeting 0≤n2+n3+n4≤5 when n1=0, and 0≤n2+n3+n4≤7 when n1=1, n5 is 0 or 1, n6 is 0, 1 or 2, n7 is 0, 1 or 2, n8 is 0, 1 or 2, meeting 0≤n6+n7+n8≤5 when n5=0, and 0≤n6+n7+n8≤7 when n5=1, n9 is 0 or 1, n10 is 0, 1 or 2, n11 is 0, 1 or 2, n12 is 0, 1 or 2, meeting 0≤n10+n11+n12≤5 when n9=0, and 0≤n10+n11+n12≤7 when n9=1, 1≤n2+n6+n10≤6, and 1≤n3+n7+n11≤6,
R¹, R² and R³ are each independently halogen, nitro, cyano, hydroxy, carboxy, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom; when n4=2, two R¹ may be identical or different and two R¹ may bond together to form a ring with the carbon atoms to which they are attached; when n8=2, two R² may be identical or different and two R² may bond together to form a ring with the carbon atoms to which they are attached; when n12=2, two R³ may be identical or different and two R³ may bond together to form a ring with the carbon atoms to which they are attached;
R^{AL1}, R^{AL2} and R^{AL3} are each independently an acid labile group; when n3=2, two R^{AL1} may be identical or different; when n7=2, two R^{AL2} may be identical or different; when n11=2, two R^{AL3} may be identical or different;
at least one —SF₅ group is attached to a carbon atom adjoining the carbon atom to which —O—R^{AL1}, —O—R^{AL2} or —O—R^{AL3} is attached,
two of three aromatic rings bonded to S⁺ may bond together to form a ring with the sulfur atom to which they are attached, and
Z⁻ is an aromatic carboxylic acid anion which may have a substituent.

2. The sulfonium salt of claim 1, having the formula (1A): 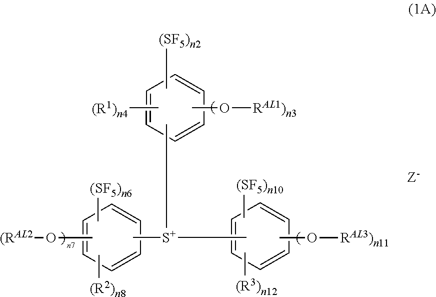
wherein n2 to n4, n6 to n8, n10 to n12, R^{AL1} to R^{AL3}, R¹ to R³, and Z⁻ are as defined above.

3. The sulfonium salt of claim 1 wherein the acid labile group represented by R^{AL1}, R^{AL2} and R^{AL3} has the formula (AL-1) or (AL-2): 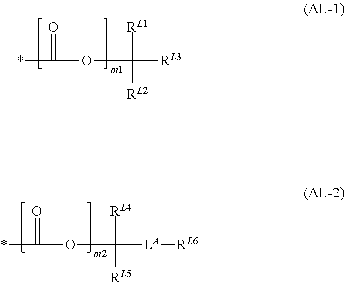
wherein m1 and m2 are each independently 0 or 1,
R^{L1} and R^{L2} are each independently a C₁-C₁₂ hydrocarbyl group, R^{L3} is hydrogen or a C₁-C₁₂ hydrocarbyl group; some —CH₂— in the hydrocarbyl group represented by R^{L1}, R^{L2} and R^{L3} may be replaced by —O— or —S—; when the hydrocarbyl group contains an aromatic ring, some or all of the hydrogen atoms on the aromatic ring may be substituted by halogen, cyano, nitro, a C₁-C₄ alkyl moiety which may contain halogen, or C₁-C₄ alkoxy moiety which may contain halogen; R^{L1} and R^{L2} may bond together to form a ring with the carbon atom to which they are attached, some —CH₂— in the ring may be replaced by —O— or —S—, with the proviso that when R^{L3} is hydrogen, R^{L1} and R^{L2} bond together to form a multiple bond-containing alicyclic structure with the carbon atom to which they are attached,
R^{L4} and R¹⁵ are each independently hydrogen or a C₁-C₁₀ hydrocarbyl group, R^{L6} is a C₁-C₂₀ hydrocarbyl group, some —CH₂— in the hydrocarbyl group may be replaced by —O—or —S—; R^{L5} and R^{L6} may bond together to form a C₃-C₂₀ heterocyclic group with the carbon atom and L^{A} to which they are attached, some —CH₂— in the heterocyclic group may be replaced by —O— or —S—,
L^{A} is —O— or —S—,
* designates a point of attachment to the adjoining —O—.

4. The sulfonium salt of claim 1 wherein Z⁻ is an anion having the formula (Z): 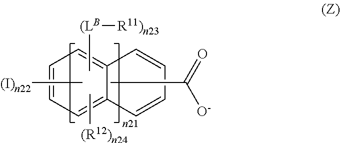
wherein n21 is 0 or 1, n22 is 0, 1, 2, 3 or 4, n23 is 0, 1 or 2, n24 is 0, 1, 2, 3 or 4, meeting 2≤n22+n23+n24≤5 when n21=0 and 2≤n22+n23+n24≤7 when n21=1,
L^{B} is a single bond, oxygen, sulfur, ester bond or carbonate bond,
R¹¹ is hydrogen or a C₁-C₂₀ hydrocarbyl group which may contain a heteroatom when L^{B} is a single bond, R¹¹ is hydrogen, a C₁-C₂₀ hydrocarbyl group (exclusive of acid labile group) which may contain a heteroatom, or acid labile group when L^{B} is oxygen, sulfur, ester bond or carbonate bond,
R¹² is halogen exclusive of iodine, nitro, cyano, pentafluorosulfanyl, a C₁-C₂₀ hydrocarbyl group which may contain a heteroatom, C₁-C₂₀ hydrocarbyloxy group which may contain a heteroatom or C₁-C₂₀ hydrocarbylthio group which may contain a heteroatom; when n24 is 2, 3 or 4, a plurality of R¹² may bond together to form a ring structure with the carbon atoms on the aromatic ring to which they are attached, some —CH₂-in the ring may be replaced by —O— or —S—.

5. The sulfonium salt of claim 4 wherein Z⁻ is an anion having the formula (Z1): 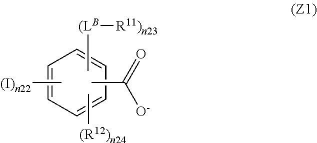
wherein n²² to n²⁴, L^{B}, R¹¹ and R¹² are as defined above.

6. The sulfonium salt of claim 4 wherein L^{B} is oxygen or ester bond.

7. The sulfonium salt of claim 4 wherein L^{B} is oxygen, sulfur, ester bond or carbonate bond and R¹¹ is an acid labile group.

8. The sulfonium salt of claim 4 wherein the acid labile group represented by R¹¹ has the formula (AL-3) or (AL-4): 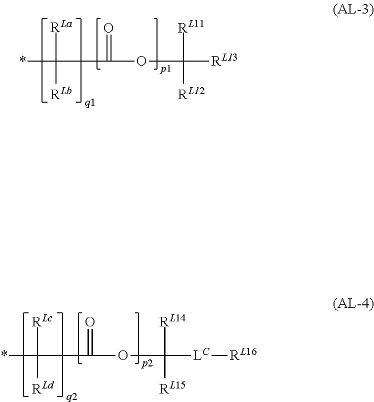
wherein p1 and p2 are each independently 0 or 1, q1 and q2 are each independently 0, 1, 2, 3 or 4,
R^{L11}, R^{L12} and R^{L13} are each independently a C₁-C₁₂ hydrocarbyl group in which some —CH₂— may be replaced by —O— or —S—; when the hydrocarbyl group contains an aromatic ring, some or all of the hydrogen atoms on the aromatic ring may be substituted by halogen, cyano, nitro, a C₁-C₄ alkyl moiety which may contain halogen, or C₁-C₄ alkoxy moiety which may contain halogen; R^{L11} and R^{L12} may bond together to form a ring with the carbon atom to which they are attached, some —CH₂— in the ring may be replaced by —O— or —S—,
R^{L14} and R^{L15} are each independently hydrogen or a C₁-C₁₀ hydrocarbyl group, R^{L16} is a C₁-C₂₀ hydrocarbyl group, some —CH₂— in the hydrocarbyl group may be replaced by —O— or —S—, R^{L15} and R^{L16} may bond together to form a C₃-C₂₀ heterocyclic group with the carbon atom and L^{C} to which they are attached, some —CH₂— in the heterocyclic group may be replaced by —O— or —S—,
L^{C} is-O— or —S—,
R^{La} to R^{Ld} are each independently hydrogen or a C₁-C₂₀ hydrocarbyl group which may contain a heteroatom, and
* designates a point of attachment to the adjoining oxygen or sulfur.

9. A quencher in the form of the sulfonium salt of claim 1.

10. A chemically amplified resist composition comprising the quencher of claim 9.

11. The resist composition of claim 10, further comprising a base polymer containing a polymer comprising repeat units of at least one type selected from repeat units having the formula (a1), repeat units having the formula (a2), and repeat units having the formula (a3): 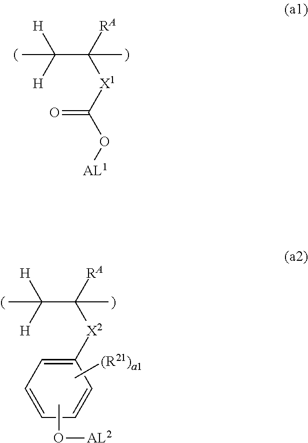
wherein R^{A} is each independently hydrogen, fluorine, methyl or trifluoromethyl,
X¹ is a single bond, phenylene group, naphthylene group, or *—C(═O)—O—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond or *—C(═O)—O—,
* designates a point of attachment to the carbon atom in the backbone,
R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and when a1 is 2, 3 or 4, a plurality of R²¹ may be identical or different,
AL¹ and AL² are each independently an acid labile group, and
a1 is 0, 1, 2, 3 or 4, 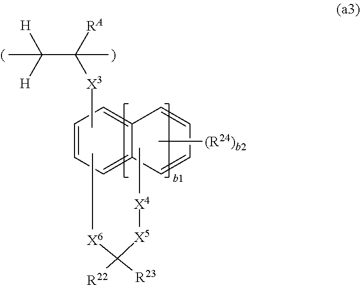
wherein b1 is 0 or 1, b2 is 0, 1, 2 or 3 when b1=0, b2 is 0, 1, 2, 3, 4 or 5 when b1=1,
R^{A} is hydrogen, fluorine, methyl or trifluoromethyl,
X³ is a single bond, *—C(═O)—O— or *—C(═O)—N(H)—, * designates a point of attachment to the carbon atom in the backbone,
X⁴ is a single bond, C₁-C₄ aliphatic hydrocarbylene group, carbonyl group, sulfonyl group or a combination thereof,
X⁵ and X⁶ are each independently oxygen or sulfur, with the proviso that X⁴ and X⁶ are attached to adjoining carbon atoms on the aromatic ring,
R²² and R²³ are each independently hydrogen or a C₁-C₂₀ hydrocarbyl group which may contain a heteroatom, R²² and R²³ may bond together to form a ring with the carbon atom to which they are attached,
R²⁴ is halogen, hydroxy, cyano, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}), R^{24A} and R^{24B} are each independently hydrogen or a C₁-C₆ hydrocarbyl group; when b2 is 2 or more, a plurality of R²⁴ may be identical or different and a plurality of R²⁴ may bond together to form a ring with the carbon atoms on the aromatic ring to which they are attached.

12. The resist composition of claim 11 wherein the polymer further comprises repeat units having the formula (b1) or (b2): 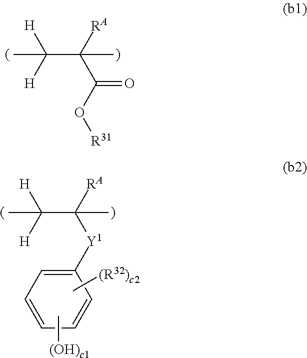
wherein R^{A} is each independently hydrogen, fluorine, methyl or trifluoromethyl,
Y¹ is a single bond or *—C(═O)—O—, * designates a point of attachment to the carbon atom in the backbone,
R³¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom; when c2 is 2, 3 or 4, a plurality of R³² may be identical or different,
c1 is 1, 2, 3 or 4, c2 is 0, 1, 2, 3 or 4, and 1≤c1+c2≤5.

13. The resist composition of claim 11 wherein the polymer further comprises repeat units of at least one type selected from repeat units having the formula (c1), repeat units having the formula (c2), repeat units having the formula (c3), repeat units having the formula (c4), and repeat units having the formula (c5): 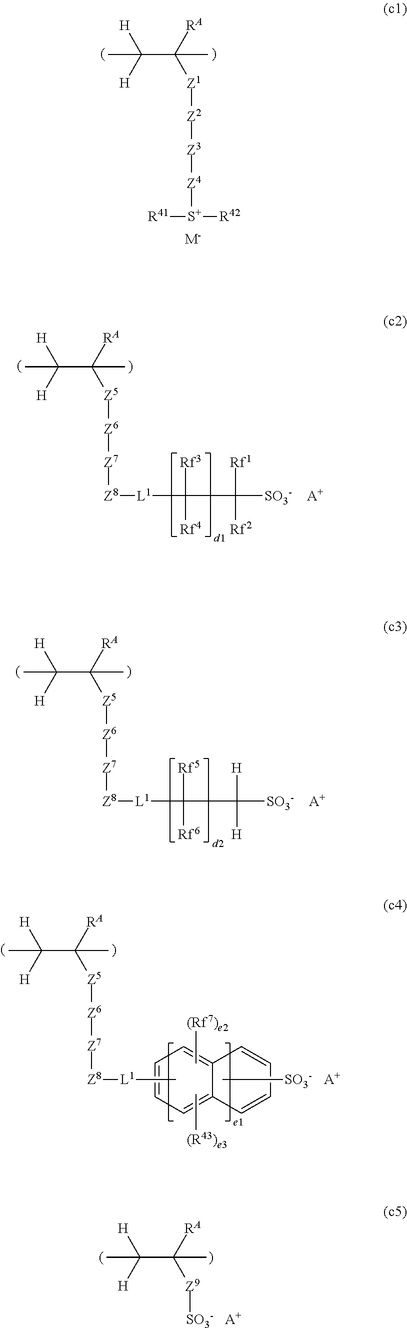
wherein d1 and d2 are each independently 0, 1, 2 or 3, e1 is 0 or 1, e2 is 0, 1, 2, 3 or 4, e3 is 0, 1, 2, 3 or 4, meeting 0≤e2+e3≤4 when e1=0 and 0≤e2+e3≤6 when e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—N(H)—Z²¹—, or **—O—Z²¹, Z²¹ is a C₁-C₆ aliphatic hydrocarbylene group, phenylene group or a divalent group obtained by combining the foregoing, which may contain halogen, carbonyl moiety, ester bond, ether bond or hydroxy moiety,
Z³ is a single bond, ether bond, ester bond, sulfonate ester bond, amide bond, sulfonamide bond, carbonate bond or carbamate bond,
Z⁴ is a single bond or a C₁-C₆ aliphatic hydrocarbylene group, phenylene group or a divalent group obtained by combining the foregoing, which may contain halogen, carbonyl moiety, ester bond, ether bond or hydroxy moiety,
Z⁵ is each independently a single bond, optionally substituted phenylene group, optionally substituted naphthylene group, *—C(═O)—O—Z⁵¹— or *—C(═O)—N(R)—Z⁵¹—, Z⁵¹ is a C₁-C₁₀ aliphatic hydrocarbylene group, phenylene group or naphthylene group, the aliphatic hydrocarbylene group may contain halogen, hydroxy, ether bond, ester bond or lactone ring, R is hydrogen or a C₁-C₁₀ hydrocarbyl group which may contain a heteroatom,
Z⁶ is a single bond, ether bond, ester bond, sulfonate ester bond, amide bond, sulfonamide bond, carbonate bond or carbamate bond,
Z⁷ is each independently a single bond, ***—Z⁷¹—C(═O)—O—, ***—C(═O)—N(R)—Z⁷¹— or ***—O—Z⁷¹—, Z⁷¹ is a C₁-C₂₀ hydrocarbylene group which may contain a heteroatom, R is as defined above,
Z⁸ is each independently a single bond, ****—Z⁸¹—C(═O)—O—, ****—C(═O)—N(R)—Z⁸¹— or ****—O—Z⁸¹—, Z⁸¹ is a C₁-C₂₀ hydrocarbylene group which may contain a heteroatom, R is as defined above,
Z⁹ is a single bond, methylene, ethylene, phenylene, fluorinated phenylene, trifluoromethyl-substituted phenylene, *—C(═O)—O—Z⁹¹—, *—C(═O)—N(R)—Z⁹¹— or *—O—Z⁹¹—, Z⁹¹ is a C₁-C₆ aliphatic hydrocarbylene group, phenylene group, fluorinated phenylene group, or trifluoromethyl-substituted phenylene group, which may contain carbonyl, ester bond, ether bond or hydroxy, R is as defined above,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, C₁-C₆ fluorinated alkoxy group, C₁-C₆ fluorinated alkylthio group, or pentafluorosulfanyl; when e2 is 2, 3 or 4, a plurality of Rf⁷ may be identical or different,
R⁴¹ and R⁴² are each independently a C₁-C₂₀ hydrocarbyl group which may contain a heteroatom, R⁴¹ and R⁴² may bond together to form a ring with the sulfur atom to which they are attached,
R⁴³ is halogen exclusive of fluorine, or a C₁-C₂₀ hydrocarbyl group which may contain a heteroatom; when e3 is 2, 3 or 4, a plurality of R⁴³ may be identical or different and a plurality of R⁴³ may bond together to form a ring with the carbon atoms to which they are attached,
M⁻ is a non-nucleophilic counter ion, and
A⁺ is an onium cation.

14. The resist composition of claim 10, further comprising an organic solvent.

15. The resist composition of claim 10, further comprising a photoacid generator capable of generating a strong acid.

16. The resist composition of claim 10, further comprising a quencher other than the quencher.

17. The resist composition of claim 10, further comprising a surfactant.

18. A pattern forming process comprising the steps of applying the chemically amplified resist composition of claim 10 onto a substrate to form a resist film thereon, exposing the resist film to high-energy radiation, and developing the exposed resist film in a developer.

19. The process of claim 18 wherein the high-energy radiation is KrF excimer laser, ArF excimer laser, EB or EUV of wavelength 3 to 15 nm.
